(19) United States
(12) Patent Application Publication
(10) Pub. No.: US 2026/0274953 A1
(43) Pub. Date: Sep. 17, 2026

(54) IMMUNOGLOBULIN SINGLE VARIABLE DOMAINS TARGETING PD-1

(71) Applicants: ABLYNX N.V., Gent (BE); SANOFI, Paris (FR)

(72) Inventors: Jérémy Baudhuin, Paris (FR); Christian Beil, Frankfurt am Main (DE); Thomas Bertrand, Gent (BE); Magdalena Bukowska, Gent (BE); Christian Lange, Frankfurt am Main (DE); Sandra Li, Gent (BE); Sevim Özgür Brüderle, Frankfurt am Main (DE); Natalia Sarmiento, Gent (BE); Ingrid Sassoon, Paris (FR); Sandra Weil, Frankfurt am Main (DE)

(21) Appl. No.: 19/427,808

(22) Filed: Dec. 19, 2025

(30) Foreign Application Priority Data
Dec. 20, 2024 (EP) 24307253.5
Dec. 20, 2024 (EP) 24307255.0

(51) Int. Cl. C07K 16/28; A61K 38/00; A61P 35/00; C12N 15/63

(52) U.S. Cl. CPC C07K 16/2818; A61K 38/00; A61P 35/00; C07K 16/2878; C12N 15/63; C07K 2317/21; C07K 2317/22; C07K 2317/24; C07K 2317/31; C07K 2317/35; C07K 2317/565; C07K 2317/567; C07K 2317/569; C07K 2317/94

(57) ABSTRACT

The present technology provides polypeptides that binds to PD-1. The present technology also provides nucleic acids, vectors and compositions. Also provided are multispecific binding proteins (e.g., bispecific binding proteins) that targeting a TNF receptor superfamily (TNFRSF) receptor (e.g., GITR) and an immune checkpoint protein (e.g., PD-1).

### RELATED APPLICATIONS

This application claims priority to European Patent Application Serial No. 24307255.0, filed on Dec. 20, 2024, and European Patent Application Serial No. 24307253.5, filed on Dec. 20, 2024, the disclosures of which are hereby incorporated by reference.

### SEQUENCE LISTING

The instant application contains a Sequence Listing which has been submitted electronically in XML format and is hereby incorporated by reference in its entirety. Said XML file, created on May 22, 2026, is named 772539_SA9-951.xml and is 4,504,175 bytes in size.

### 1 FIELD OF THE PRESENT TECHNOLOGY

The present technology relates to immunoglobulin single variable domains (ISVDs) targeting PD-1 and more in particular to polypeptides and constructs that comprise or essentially consist of one or more such ISVDs. It also relates to nucleic acid molecules encoding the ISVDs and polypeptides and vectors comprising the nucleic acids, and to compositions comprising the ISVD, polypeptide, nucleic acid or vector. The present technology further relates to these products for use in a method of treating a subject suffering from cancer. The present technology also relates to methods of producing these products.

### 2 TECHNOLOGICAL BACKGROUND

The PD-1 (programmed cell death-1) receptor (CD279) is expressed on the surface of activated T cells. Its ligands, PD-L1 (B7-H1; CD274) and PD-L2 (B7-DC; CD273), are transmembrane proteins of the B7 family that are commonly expressed on dendritic cells and macrophages. PD-1 and PD-L1/PD-L2 belong to the family of immune checkpoint proteins that act as co-inhibitory factors that can halt or limit the development of the T-cell response. The PD-1/PD-L1 interaction ensures that the immune system is activated only at the appropriate time to minimize the possibility of chronic autoimmune inflammation. The biology of PD-L1 and its role in cancer immunology and immunotherapy are described by Sun et al. 2018 (Immunity 48:434) and Salmaninejad et al. 2019 (J. Cell Physiol. 234: 16824).

A number of biological compounds aiming at blocking the PD-1/PD-L1 interaction have already been approved for use in cancer. Pembrolizumab (Keytruda; Merck) and Nivolumab (Opdivo; Bristol-Myers Squibb) are monoclonal anti-PD-1 lg4 antibodies, both of which were approved by the FDA in 2014 for the treatment of melanoma, followed by the approval for various other cancers including multiple melanoma, non-small cell lung cancer (NSCLC), liver cancer, and colorectal cancer. Cemiplimab (Libtayo; Regeneron Pharmaceuticals) is an immunoglobulin (lg) G4 monoclonal antibody (mAb) to the programmed cell death (PD)-1 which was the first FDA-approved treatment for advanced cutaneous squamous cell carcinoma and was later approved to be used in basal cell carcinoma and non-small cell lung cancer. Several other monoclonal anti-PD-1 antibodies have been approved since then.

The less efficient penetration of monoclonal antibodies because of their large size and the complexity of the tumor microenvironment has impelled the development of small-molecule biologics with superior penetration efficiencies. Envafolimab (KN035; 3D Medicines/Alphamab Oncology/Simcere Pharmaceutical Group) is an anti-PD-L1 single domain-Fc fusion protein and is currently marketed for the treatment of advanced biliary cancer and soft tissue sarcoma. Immunoglobulin single variable domains that target PD1 have been described in WO 2008/071447.

Hence there is a need for improved small-molecule biologics to PD-1. Such small-molecule biologics are also useful for combining with other (cancer) target binders in a single chain, while maintaining a relatively small size, high affinity, potency and/or efficacy, in addition to good manufacturing yields.

Furthermore, the biological drug should show safety, convenience, patient compliance and improve patients' quality of life.

### 3 SUMMARY OF THE PRESENT TECHNOLOGY

In some embodiments, the present technology relates to immunoglobulin single variable domains specifically targeting PD-1. In some embodiments, the present technology relates to high affinity immunoglobulin single variable domains specifically targeting PD-1. In some embodiments, the present technology relates to high potency immunoglobulin single variable domains specifically targeting PD-1. In some embodiments, the present technology relates to high efficacy immunoglobulin single variable domains specifically targeting PD-1.

Targeting PD-1 with a biologic that is small (in size), such as a monovalent or bivalent immunoglobulin single variable domain, and keeping a high affinity, potency and/or efficacy may be advantageous in certain applications where a very small binder is desired. Small binders may be advantageous, e.g. for penetration in the tumor environment, or when additional binders for other (tumor) targets are implicated in the same construct, facilitating manufacturing and further handling of the therapeutic compound.

The present technology provides immunoglobulin single variable domains specifically targeting PD-1 that block the interaction of PD1 and PD-L1 (with comparable or better EC50 than benchmark(s)). The present technology provides immunoglobulin single variable domains specifically targeting PD-1 that show potency in bioassays, such as binding of PD-1 and competition with PD-L1 in flow-cytometry, or functionality in a tetanus-toxoid autogenic antigen-specific co-culture assay (tetanus-toxoid (TT) assay) and NFAT PD1/PD-L1 blocking assay.

The immunoglobulin single variable domains of the present technology and polypeptides and constructs comprising said immunoglobulin single variable domains showed a high potency in competition assays with PD-L1 and a high potency and efficacy in TT assay, and could be efficiently produced (e.g. in microbial hosts such as Pichia, e.g. P. pastoris).

In one embodiment, the immunoglobulin single variable domain, polypeptide or construct of the present technology may further comprise one or more other groups, residues, moieties or binding units, optionally linked via one or more peptidic linkers, in which said one or more other groups, residues, moieties or binding units provide the ISVD, polypeptide or construct with increased half-life, compared to the corresponding ISVD, polypeptide or construct without said one or more other groups, residues, moieties or binding units. Said one or more other groups, residues, moieties or binding units that provide the ISVD, polypeptide or construct with increased half-life may be chosen from the group consisting of a polyethylene glycol molecule (PEG), serum proteins or fragments thereof, binding units that can bind to serum proteins, an Fc portion, and small proteins or peptides that can bind to serum proteins. The binding units that provide the ISVD, polypeptide or construct with increased half-life may be chosen from the group consisting of binding units that can bind to serum albumin (such as human serum albumin) or a serum immunoglobulin (such as lgG), e.g., human serum albumin.

In one embodiment, the binding unit that can bind to serum albumin (such as human serum albumin) is an ISVD. In one embodiment, the ISVD binding to human serum albumin essentially consists of 4 framework regions (FR1 to FR4, respectively) and 3 complementarity determining regions (CDR1 to CDR3 respectively), in which CDR1 is SEQ ID NO: 2436, CDR2 is SEQ ID NO: 2438, CDR3 is SEQ ID NO: 2440. In one embodiment, the ISVD binding to human serum albumin comprises a sequence identity of more than 90% (such as 95%) with SEQ ID NO: 2416. In one embodiment, the ISVD binding to human serum albumin comprises or consists of the amino acid sequence of SEQ ID NO: 2416.

In one embodiment, the ISVD binding to human serum albumin is selected from the group consisting of ALB8 (SEQ ID NO: 2401), ALB23 (SEQ ID NO: 2402), ALBX00001 (SEQ ID NO: 2415) and ALB23002 (SEQ ID NO: 2416). In one embodiment, the ISVD binding to human serum albumin is ALB23002 (SEQ ID NO: 2416).

Also provided is a nucleic acid molecule capable of expressing the immunoglobulin single variable domain or polypeptide of the present technology, a vector comprising the nucleic acid, and a composition comprising the polypeptide, nucleic acid or vector.

Also provided is a (non-human) host or host cell comprising such a nucleic acid.

Also provided is a method for producing an immunoglobulin single variable domain or polypeptide of the present technology, said method at least comprising the steps of:
a) expressing, in a suitable host cell or (non-human) host organism or in another suitable expression system, a nucleic acid encoding the immunoglobulin single variable domain or polypeptide of the present technology; optionally followed by:
b) isolating and/or purifying the ISVD or polypeptide.

Also provided is a composition comprising at least one immunoglobulin single variable domain, polypeptide or construct of the present technology, or a nucleic acid encoding an immunoglobulin single variable or polypeptide of the present technology.

The composition of the present technology is for use as a medicament. The composition can be a pharmaceutical composition which further comprises at least one pharmaceutically acceptable carrier, diluent or excipient and/or adjuvant, and optionally comprises one or more further pharmaceutically active polypeptides and/or compounds.

The immunoglobulin single variable domain, polypeptide or construct of the present technology, a composition comprising the immunoglobulin single variable domain, polypeptide or construct, and a composition comprising a nucleic acid comprising a nucleotide sequence that encodes the immunoglobulin single variable domain or polypeptide can be used as a medicament.

The immunoglobulin single variable domain, polypeptide, construct or composition of the present technology can be used in the treatment. More specifically, the immunoglobulin single variable, polypeptide, construct or composition can be used in the treatment of cancer.

Accordingly, the present technology also encompasses a method of treating cancer. In some embodiments, the present technology encompasses a method of treating cancer, wherein said method comprises administering, to a subject in need thereof, a pharmaceutically active amount of an immunoglobulin single variable domain, polypeptide or construct of the present technology, a nucleic acid encoding an ISVD or polypeptide of the present technology or a composition comprising the same.

Accordingly, the present technology also encompasses the use of an immunoglobulin single domain, polypeptide or construct of the present technology in the preparation of a pharmaceutical composition for treating cancer.

### 4 BRIEF DESCRIPTION OF THE DRAWINGS

FIGS. 1A-1B. Crystal structure of A0294047E02 in complex with PD-1. (FIG. 1A) Whole complex of A0294047E02 with PD-1; (FIG. 1B) B-sheet formed by the interactions of CDR3 with a β-strand from hPD1.

FIGS. 2A-2B. Detailed view of sites in the interaction between the PD-1 and A0294047E02 of one of the complexes. (FIG. 1A) Key interacting residues of paratope on A0294047E02 (chain H) located at <4.00 Å distance from PD-1 (chain A). (FIG. 2B) Key interacting residues of epitope on PD-1 (chain A) located at <4.0 Å distance from A0294047E02 (chain H).

FIG. 3. Crystal structure of A02900073 in complex with PD-1.

FIGS. 4A-4B. Detailed view of sites in the interaction between the PD-1 and A02900073 of one of the complexes. (FIG. 4A) Key interacting residues of paratope on A02900073 (chain H) located at <4.00 Å distance from the PD-1 (chain A). (FIG. 4B) Key interacting residues of epitope on PD-1 (chain A) located at <4.0 Å distance from A02900073 (chain H).

FIGS. 5A-5B. Crystal structure of A02900072 in complex with PD-1. (FIG. 5A) Whole complex of A02900072 with PD-1; (FIG. 5B) Surface representation of the interaction between chain A and chain C.

FIGS. 6A-6B. Detailed view of sites in the interaction between the PD-1 and A02900072 of one of the complexes. (FIG. 6A) Key interacting residues of paratope on A02900072 (chain C) located at <4.00 Å distance from the hPD-1 (chain A). (FIG. 6B) Key interacting residues of epitope on hPD-1 (chain A) located at <4.0 Å distance from A02900072 (chain C).

FIG. 7. Binding mode comparison between A0294047E02, A029400073 and A029400072.

FIGS. 8A-8B. Comparison of the binding modes of the anti-PD-1 ISVDs to PD-1 (UniProt: Q15116; SEQ ID NO: 4703). (FIG. 8A) Alignment of the epitopes bound on PD-1. Epitopes are indicated as bold underlined; (FIG. 8B) Alignment of the anti-PD-1 ISVDs. Paratopes are indicated as bold underlined.

FIG. 9. Binding mode comparison of the anti-PD-1 ISVDs with PD-L1.

FIGS. 10A-10B are schematic drawings of a pentavalent PD-1/GITR bispecific binding protein described in the disclosure (also referred to herein as “pentavalent anti-PD-1/GITR construct 1 or 2”; see Table 4). FIG. 10A shows the pentavalent anti-PD-1/GITR construct comprising a trivalent anti-human GITR (hGITR) ISVD as a GITR co-stimulatory receptor agonist and a bivalent anti-human PD-1 (hPD-1) ISVD as a PD-1 co-inhibitory receptor antagonist each linked to the N-terminus of a first and second Fc heavy chain. The two Fc heavy chains of the first and the second immunoglobulin G (lgG) chains are connected to each other via disulfide bridges. FIG. 10B details additional features of the pentavalent anti-PD-1/GITR construct described in FIG. 10A including: (a) the orientation of the binding domains with respect to the first and the second IgG, i.e., the trivalent anti-hGITR domains are linked to the N-terminal end of the first Fc heavy chain and the bivalent anti-hPD-1 domains are linked to the N-terminal end of the second Fc heavy chain; (b) the multivalent binding domains are connected by flexible linkers (e.g., a Gly4Ser (SEQ ID NO: 2419) linker); (c) an effector-null Fc domain (with substitutions at L234A and L235A in each IgG domain; also called “LALA substitutions”); (d) wherein the Fc domain is further modified with amino acid substitutions H435R and a Y436F in the second lgG domain to ablate protein A binding in one arm of the heterodimer to facilitate bispecific antibody purification (also called the “RF substitutions”); and (d) Fc-knob-in-hole engineering to facilitate dimerization of the first and second IgG domains.

FIG. 11A is a schematic drawing of an octavalent PD-1/GITR bispecific binding protein described in the disclosure (also referred to herein as, “octavalent anti-PD-1/GITR construct”; see Table 4) comprising trivalent anti-hGITR ISVDs and an anti-hPD-1 ISVD linked to a first and a second Fc heavy chain. The two Fc heavy chains each IgG chain are connected to each other via disulfide bridges. FIG. 11B details additional features the octavalent anti-PD-1/GITR construct described in FIG. 11A including: (a) the orientation of the binding domains with respect to a first and a second IgG domain—the trivalent anti-hGITR domains are linked to the C-terminus of a first Fc heavy chain and a single anti-hPD-1 domain is linked to the N-terminus of a first Fc heavy chain where the second IgG comprises the same orientation and domains; (b) the domains are connected by flexible linkers, including an extended GlySer linker (e.g., (Gly₄Ser)₄) (SEQ ID NO: 2426) to connect the C-terminal end of Fc heavy chain to the anti-GITR domain; and (c) an effector-null Fc domain with LALA substitution.

FIG. 12A is a graph quantifying by FACS the binding to hPD-1 expressed on pre-B 300.19 cells upon incubation with serial dilutions of either pentavalent anti-PD-1/GITR construct 1 or 2 (filled circles and squares) or controls. FIG. 12B is the same experimental set-up as FIG. 12A but quantifies the binding to cynomolgus monkey PD-1 (cynoPD-1) expressed on pre-B 300.19 cells.

FIG. 13A is the same experimental set-up as FIG. 12A but with incubation with the octavalent anti-PD-1/GITR construct (filled circles) or controls. FIG. 13B is the same experimental set-up as FIG. 13A but quantifies the binding to cynoPD-1 expressed on pre-B 300.19 cells.

FIG. 14A is a graph quantifying by FACS the binding to hGITR expressed on NFkB-NlucP/HEK293 cells upon incubation with serial dilutions of either pentavalent anti-PD-1/GITR construct 1 or 2 (filled circle and squares) or controls. FIG. 14B is the same experimental set-up as FIG. 14A but quantifies the binding to cynoGITR expressed on NFkB-NlucP/HEK293 cells.

FIG. 15A is a graph quantifying by FACS the binding to hGITR expressed on NFkB-NlucP/HEK293 cells upon incubation with serial dilutions of either octavalent anti-PD-1/GITR construct (filled circles) or controls. FIG. 15B is the same experimental set-up as FIG. 15A but quantifies the binding to cynoGITR expressed on NFkB-NlucP/HEK293 cells.

FIG. 16A is a graph quantifying by FACS the binding to activated human primary T cells (which expressed both PD-1 and GITR) upon incubation with escalating concentrations of either pentavalent (filled circles and square) or octavalent (filled diamonds) anti-PD-1/GITR constructs or controls. FIG. 16B is the same experimental set-up as FIG. 16A but quantifies the binding to activated cynomolgus monkey primary T cells (cyno primary T cells).

FIG. 17A analyzes the activity of GITR after incubation of a GloResponse™ NFkB-NlucP/HEK293 cell line which was stably transfected to express hGITR with either serial dilutions of the pentavalent anti-PD-1/GITR construct 1 or 2 (filled circles and squares), the octavalent anti-PD-1/GITR construct (filled diamonds) or with controls. FIG. 17B is the same experimental set-up as FIG. 17A but where the cell line was stably transfected to express cynoGITR.

FIG. 18 analyzes the activity of human PD-1 after incubation of a GloResponse™ NFAT-RE/PD-1 Jurkat cell line was incubated with either serial dilutions of the pentavalent anti-PD-1/GITR construct 1 or 2 (filled circles and squares), the octavalent anti-PD-1/GITR construct (filled diamonds), or with controls.

FIGS. 19A-19B analyze the surface occupancy of each binding moiety, human PD-1 (FIG. 19A) and human GITR (FIG. 19B), on splenic activated CD4⁺ and CD8⁺ T cells (filled circles and triangles, respectively) harvested from hGITR/hPD-1 KI mice upon incubation with serial dilutions of the pentavalent anti-PD-1/GITR construct 1.

FIGS. 20A-20C analyze the activation of primary human CD3⁺ T cells isolated from human PBMC donors (three different donors were used: D1475, D1735, and D1743) via IL-2 release (pg/ml) upon incubation with either serial dilutions of the pentavalent anti-PD-1/GITR construct 1 or 2 (filled circles and squares) or with controls.

FIGS. 21A-21C are the same experimental set-up as FIGS. 20A-20C but incubation is performed with either serial dilutions of the octavalent anti-PD-1/GITR construct (filled circle) or controls.

FIGS. 22A-22C measure the results of a mixed lymphocyte reaction (MLR) assay via human GM-CSF secretion (pg/ml) into the cell medium from an immature dendritic cell (iDC) co-culture with autologous CD4⁺ T cells derived from three different PBMC donors (D1014, D1013, and D1015) upon incubation with either serially diluted pentavalent anti-PD-1/GITR construct 1 or with controls.

FIGS. 23A-23C are the same experimental set-up as FIGS. 22A-22C but upon incubation with either serially diluted pentavalent anti-PD-1/GITR construct 2 or with controls.

FIGS. 24A-24C are the same experimental set-up as FIGS. 22A-22C and 23A-23C but upon incubation with either serially diluted the octavalent anti-PD-1/GITR construct or with controls.

FIG. 25 analyzes the anti-tumor efficacy of a pentavalent anti-PD-1/GITR construct 1 compared to controls in hPD-1×hGITR double KI mice bearing sub-cutaneous MC38 established tumors. Median tumor volume±median average deviation (MAD).

FIG. 26 shows graphs of the dose-dependent anti-tumor efficacy of a pentavalent anti-PD-1/GITR construct 1 in hPD-1×hGITR double KI mice bearing sub-cutaneous MC38 established tumors. Individual curves are shown.

### 5 DETAILED DESCRIPTION OF THE PRESENT TECHNOLOGY

### 5.1 Definitions

Unless indicated or defined otherwise, all terms used have their usual meaning in the art, which will be clear to the skilled person. Reference is for example made to the standard handbooks, such as Sambrook et al. (Molecular Cloning: A Laboratory Manual (2nd.Ed.) Vols. 1-3, Cold Spring Harbor Laboratory Press, 1989), F. Ausubel et al. (Current protocols in molecular biology, Green Publishing and Wiley Interscience, New York, 1987), Lewin (Genes II, John Wiley & Sons, New York, N.Y., 1985), Old et al. (Principles of Gene Manipulation: An Introduction to Genetic Engineering (2nd edition) University of California Press, Berkeley, CA, 1981); Roitt et al. (Immunology (6th. Ed.) Mosby/Elsevier, Edinburgh, 2001), Roitt et al. (Roitt's Essential Immunology (10th Ed.) Blackwell Publishing, U K, 2001), and Janeway et al. (Immunobiology (6th Ed.) Garland Science Publishing/Churchill Livingstone, New York, 2005), as well as to the general background art cited herein.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta (Adv. Drug Deliv. Rev. 58 (5-6): 640-56, 2006), Levin and Weiss (Mol. Biosyst. 2 (1): 49-57, 2006), Irving et al. (J. Immunol. Methods 248 (1-2): 31-45, 2001), Schmitz et al. (Placenta 21 Suppl. A: S106-12, 2000), Gonzales et al. (Tumour Biol. 26 (1): 31-43, 2005), which describe techniques for protein engineering, such as affinity maturation and other techniques for improving the specificity and other desired properties of proteins such as immunoglobulins.

The term “sequence” as used herein (for example in terms like “immunoglobulin sequence”, “antibody sequence”, “variable domain sequence”, “VHH sequence” or “protein sequence”), should generally be understood to include both the relevant amino acid sequence as well as nucleic acids or nucleotide sequences encoding the same, unless the context requires a more limited interpretation.

Amino acid residues will be indicated according to the standard three-letter or one-letter amino acid code. Reference is made to Table A-2 on page 48 of WO 08/020079.

A nucleic acid or amino acid is considered to be “(in) (essentially) isolated (form)”—for example, compared to the reaction medium or cultivation medium from which it has been obtained-when it has been separated from at least one other component with which it is usually associated in said source or medium, such as another nucleic acid, another protein/polypeptide, another biological component or macromolecule or at least one contaminant, impurity or minor component. In particular, a nucleic acid or amino acid is considered “(essentially) isolated” when it has been purified at least 2-fold, in particular at least 10-fold, more in particular at least 100-fold, and up to 1000-fold or more. A nucleic acid or amino acid that is “in (essentially) isolated form” is preferably essentially homogeneous, as determined using a suitable technique, such as a suitable chromatographical technique, such as polyacrylamide-gel electrophoresis.

When a nucleotide sequence or amino acid sequence is said to “comprise” another nucleotide sequence or amino acid sequence, respectively, or to “essentially consist of” another nucleotide sequence or amino acid sequence, this may mean that the latter nucleotide sequence or amino acid sequence has been incorporated into the first mentioned nucleotide sequence or amino acid sequence, respectively, but more usually this generally means that the first mentioned nucleotide sequence or amino acid sequence comprises within its sequence a stretch of nucleotides or amino acid residues, respectively, that has the same nucleotide sequence or amino acid sequence, respectively, as the latter sequence, irrespective of how the first mentioned sequence has actually been generated or obtained (which may for example be by any suitable method described herein). By means of a non-limiting example, when a polypeptide is said to comprise an immunoglobulin single variable domain, this may mean that said immunoglobulin single variable domain sequence has been incorporated into the sequence of the polypeptide, but more usually this generally means that the polypeptide contains within its sequence the sequence of the immunoglobulin single variable domains irrespective of how said polypeptide has been generated or obtained. Also, when a nucleic acid or nucleotide sequence is said to comprise another nucleotide sequence, the first mentioned nucleic acid or nucleotide sequence is preferably such that, when it is expressed into an expression product (e.g. a polypeptide), the amino acid sequence encoded by the latter nucleotide sequence forms part of said expression product (in other words, that the latter nucleotide sequence is in the same reading frame as the first mentioned, larger nucleic acid or nucleotide sequence).

By “(essentially) consist of” is meant that the later nucleic acid sequence or amino acid sequence, either is exactly the same as the polypeptide (e.g. the CDR region; the ISVD) or corresponds to the polypeptide (e.g. the CDR region; the ISVD) which has a limited number of amino acid residues, such as 1-20 amino acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxy terminal end of the immunoglobulin single variable domain.

For the purposes of comparing two or more amino acid sequences, the percentage of “sequence identity” between a first amino acid sequence and a second amino acid sequence may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (i.e. at a single position). Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

An “amino acid difference” as used herein refers to a deletion, insertion or substitution of a single amino acid residue vis-à-vis a reference sequence. In one embodiment, an “amino acid difference” is a substitution. In one embodiment, amino acid substitutions are conservative substitutions. Such conservative substitutions are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp. In one embodiment, conservative substitutions are as follows: Ala into Gly or into Ser; Arg into Lys; Asn into GIn or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

A “VHH family” as used in the present specification refers to a group of VHH sequences that have identical lengths (i.e. they have the same number of amino acids within their sequence) and of which the amino acid sequence between position 8 and position 106 (according to Kabat numbering) has an amino acid sequence identity of 89% or more.

The terms “epitope” and “antigenic determinant”, which can be used interchangeably, refer to the part of a macromolecule, such as a polypeptide or protein that is recognized by antigen-binding molecules, such as immunoglobulins, conventional antibodies, or immunoglobulin single variable domains, and more particularly by the antigen-binding site of said molecules. Epitopes define the minimum binding site for an immunoglobulin, and thus represent the target of specificity of an immunoglobulin. Epitopes may be either conformational or linear. A conformational epitope is produced by spatially juxtaposed amino acids from different segments of the linear polypeptide chain. A linear epitope is one produced by adjacent amino acid residues in a polypeptide chain. As will be appreciated by one of skill in the art, practically anything to which an antibody can specifically bind could be an epitope. The definition of the term “paratope” is derived from the above definition of “epitope”, by reversing the perspective. Thus, the term “paratope” refers to the area or region (i.e., the residues) of an antibody or fragment thereof involved in recognition of and binding to the epitope of an antigen.

The term “interact with” as used herein in the context of at least two polypeptides forming a complex (e.g. anti-PD-1 ISVD and PD-1) means that at least one (amino acid) residue of one polypeptide is in close proximity to at least one (amino acid) residue of the other polypeptide. The distance between two (amino acid) residues which are located within distinct polypeptides may be determined using methods known is the art. For example, the skilled person knows that structural information allowing to determine the distance between two (amino acids) residues may be obtained using standard methods such as X-ray crystallography, Cryogenic electron microscopy (cryo-EM), nuclear magnetic resonance, and subsequent molecular modelling. The skilled person is also aware that two (amino acid) residues are in close proximity and therefore interact with each other if the (shortest) distance between the two (amino acid) residues is less than 10 Å, less than 8 Å, less than 6 Å, less than 5 Å, less than 4 Å, less than 3 Å, less than 2 Å, preferably less than 4 Å, more preferably less than 4 Å and more than 2 Å.

The term “interaction site” as used herein refers to the area within a complex comprising at least two polypeptides which is formed by the (amino acid) residue(s) within each of the respective polypeptides that interact with each other as defined herein. For example, an ISVD specifically binding to PD-1 comprises at least one amino acid residue that interacts with at least one (amino acid) residue on PD-1. In this respect, the at least one amino acid residue of the anti-PD-1 ISVD and the at least one (amino acid) residue on PD-1 interacting with each other also form an interaction site. The skilled person is aware that interaction sites may for example be defined by reference to a specific amino acid residue of the polypeptide (e.g. E104 of PD-1).

Molecular interactions can have a different strength, bond length (Å), interaction area (Å2), depending on the type of amino acid residues that make up the interaction. Strong molecular interactions include hydrogen bonds (or H-bonds), that are electrostatic forces of attraction between a hydrogen (H) atom which is covalently bonded to a more electronegative donor atom or group, and another electronegative atom bearing a lone pair of electrons, the hydrogen bond acceptor. Such an interacting system is generally denoted “donor-H . . . acceptor”, where the solid line denotes a polar covalent bond, and the dotted or dashed line indicates the hydrogen bond. The most frequent donor and acceptor atoms are the period 2 elements: nitrogen (N), oxygen (O), and fluorine (F). The hydrogen bonds have a length around 1.5-2.5 Å and an interaction area around 10-50 Å². Typical amino acid residues that form a H-bond are Lys, Arg, Asn, Gln, Ser and Thr (donors), and Asp, Glu, Asn, Gln, Ser and Thr (acceptors).

Other strong molecular interactions include salt bridges, that are defined as electrostatic interactions between two oppositely charged groups: the anionic carboxylate of either glutamate (E) or aspartate (D), and the cationic ammonium from either arginine (R) or lysine (K). The salt bridges have a length around 2.8-4.0 Å and an interaction area around 10-100 Å². Typical amino acid residues that form a salt bridge are Lys, Arg and His (positive), and Asp and Glu (negative).

Weaker interactions include hydrophobic interactions, that are the non-covalent force where nonpolar species tend to cluster in water in order to decrease the overall interfacial area between the hydrophobic species and water. Hydrophobic interactions have a length around 3.5-4.5 Å and an interaction area around 100-300 Å2. Typical amino acid residues that form hydrophobic interactions are Ala, Val, Leu, Ile, Met, Phe, Trp.

Van der Waals forces include attraction and repulsions between atoms, molecules, as well as other (weaker) intermolecular forces. They differ from covalent and ionic bonding in that they are caused by correlations in the fluctuating polarizations of nearby particles. The force results from a transient shift in electron density. Specifically, the electron density may temporarily shift to be greater on one side of the nucleus. This shift generates a transient charge which a nearby atom can be attracted to or repelled by. Van der Waals forces have a length around 3.5-4.5 Å and an interaction area around 50-200 Å2.

Molecular modelling of the interaction site can be performed by tools known in the art, such as e.g., PDBe-PISA. PDBe-PISA is an interactive tool for the exploration of macromolecular interfaces based on cryo-EM or X-ray structures (https://www.ebi.ac.uk/pdbe/pisa/). PDBe-PISA represents a systematic approach to automatic identification of probable quaternary structures, based on physical-chemical models of macromolecular interactions and chemical thermodynamics. Amongst others, PDBe-PISA calculates buried surface area (BSA) according to cryoEM/Xray structures, which is the portion of molecular surfaces no longer exposed to solvent upon complex formation. Core interacting residues are deeply buried, have a high buried surface area (BSA) and low solvent exposure (ASA, Accessible Surface Area). Chemical stability of macromolecular complexes is estimated in Gibbs free energy terms (Interface free energy (AG)) which, when negative, indicates a thermodynamically stable interaction.

A polypeptide (such as an immunoglobulin, an antibody, an immunoglobulin single variable domain, or generally an antigen binding molecule or a fragment thereof) that can “bind to” or “specifically bind to”, that “has affinity for” and/or that “has specificity for” a certain epitope, antigen or protein (or for at least one part, fragment or epitope thereof) is said to be “against” or “directed against” said epitope, antigen or protein or is a “binding” molecule with respect to such epitope, antigen or protein, or is said to be “anti”-epitope, “anti”-antigen or “anti”-protein (e.g., “anti”-PD-1).

The terms “specificity”, “binding specifically” or “specific binding” refer to the number of different target molecules, such as antigens, from the same organism to which a particular binding unit, such as an ISVD, can bind with sufficiently high affinity (see below). “Specificity”, “binding specifically” or “specific binding” are used interchangeably herein with “selectivity”, “binding selectively” or “selective binding”. Binding units, such as ISVDs, specifically bind to their designated targets. The specificity/selectivity of a binding unit can be determined based on affinity. The affinity denotes the strength or stability of a molecular interaction. The affinity is commonly given by the KD, or dissociation constant, which has units of mol/liter (or M). The affinity can also be expressed as an association constant, KA, which equals 1/KD and has units of (mol/liter)⁻¹ (or M⁻¹).

The affinity is a measure for the binding strength between a moiety and a binding site on the target molecule: the lower the value of the KD, the stronger the binding strength between a target molecule and a targeting moiety. Typically, binding units used in the present technology, such as ISVDs, will bind to their targets with a dissociation constant (KD) of 10⁻⁵ to 10⁻¹² moles/liter or less, 10⁻⁷ to 10⁻¹² moles/liter or less, or 10⁻⁸ to 10⁻¹² moles/liter (i.e. with an association constant (KA) of 10⁵ to 10¹² liter/moles or more, 10⁷ to 10¹² liter/moles or more, or 10⁸ to 10¹² liter/moles). Any KD value greater than 10⁻⁴ mol/liter (or any KA value lower than 10⁴ liters/mol) is generally considered to indicate non-specific binding. The KD for biological interactions, such as the binding of immunoglobulin sequences to an antigen, which are considered specific are typically in the range of 10⁻⁵ moles/liter (10000 nM or 10 μM) to 10⁻¹² moles/liter (0.001 nM or 1 pM) or less.

Accordingly, specific/selective binding may mean that-using the same measurement method, e.g. SPR-a binding unit (or polypeptide comprising the same) binds to PD-1 with a KD value of 10⁵ to 10⁻¹² moles/liter or less and binds to related CD28 receptor family members with a KD value greater than 10⁻⁴ moles/liter. An example of a related CD28 receptor family member is CD28, CTLA-4, ICOS, and BTLA. Thus, in an embodiment of the present technology, the ISVD binds to (human) PD-1 with a KD value of 10⁻⁵ to 10⁻¹² moles/liter or less and binds to CD28, CTLA-4, ICOS, and BTLA of the same species with a KD value greater than 10⁻⁴ moles/liter.

Specific binding to a certain target from a certain species does not exclude that the binding unit can also specifically bind to the analogous target from a different species. For example, specific binding to human PD-1 does not exclude that the binding unit or a polypeptide comprising the same can also specifically bind to PD-1 from cynomolgus monkeys (“cyno”).

Specific binding of a binding unit to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned further herein.

The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more than 10⁻⁴ moles/liter or 10³ moles/liter (e.g. of 10⁻² moles/liter). Optionally, as will also be clear to the skilled person, the (actual or apparent) dissociation constant may be calculated on the basis of the (actual or apparent) association constant (KA), by means of the relationship [KD=1/KA].

An amino acid sequence is said to be “cross-reactive” for two different antigens or antigenic determinants (such as e.g., serum albumin from two different species of mammal, such as e.g., human serum albumin and cyno serum albumin, such as e.g., PD-1 from different species of mammal, such as e.g., human PD-1, cyno PD-1 and mouse PD-1) if it is specific for (as defined herein) these different antigens or antigenic determinants.

The terms “block”, “antagonize”, “compete”, “competing” and “-competition” are used interchangeably herein to mean the ability of an immunoglobulin, antibody, immunoglobulin single variable domain, polypeptide or other binding agent to interfere with the binding of another protein, polypeptides, ligand or binding agent to a given target. The extent to which an immunoglobulin, antibody, immunoglobulin single variable domain, polypeptide or other binding agent is able to interfere with the binding of another ligand to the target, and therefore whether it can be said to “block”, can be determined using competition binding assays. Particularly suitable quantitative competitive blocking assays are described in the Examples and include, e.g., a fluorescence-activated cell sorting (FACS) competition assay with PD-1 expressed on cells. The extent of blocking can be measured by the (reduced) channel fluorescence.

The following generally describes a suitable FACS assay for determining whether an immunoglobulin, antibody, immunoglobulin single variable domain, polypeptide or other binding agent blocks or is capable of blocking. It will be appreciated that the assay can be used with any of the immunoglobulin single variable domains and polypeptides described herein. The FACS instrument (e.g. FACS Canto; Becton Dickinson) is operated in line with the manufacturer's recommendations.

To evaluate the “blocking” or “competition” between two binding agents (such as an immunoglobulin single variable domain and a natural ligand or another binding agent) for binding PD-1, a FACS competition experiment can be performed using cells (such as, e.g., Flp-In™-293 cells) overexpressing human PD-1 and the parental cells as background cell line. Different detection reagents can be used including, e.g., monoclonal ANTI-FLAG® M2 antibody (Sigma-Aldrich, cat #F1804), monoclonal anti-C-myc antibody (Sigma-Aldrich, cat #WH0004609M2), monoclonal ANTI-HIS TAG antibody (Sigma-Aldrich, cat #SAB1305538), each labeled differently. A wide range of fluorophores can be used as labels in flow cytometry (such as, e.g., PE (R-Phycoerythrin), 7-aminoactinomycin D (7-AAD), Acridine Orange, various forms of Alexa Fluor, Allophycocyanin (APC), AmCyan, Aminocoumarin, APC Cy5, APC Cy7, APC-H7, APC/Alexa Fluor 750, AsRed2, Azami-Green, Azurite, B ODIPY FL C5-ceramide, BCECF-AM, Bis-oxonol DiBAC2 (3), BODIPY-FL, Calcein, Calcein AM, Caroxy-H2DCFDA, Cascade Blue, Cascade Yellow, Cell Tracker Green, Cerulean, CFSE, Chromomycin A3, CM-H2DCFDA, Cy2, Cy3, Cy3.5, Cy3B, Cy5, Cy5.5, Cy7, CyPet, DAF-FM DAF-FM diacetate, DAPI, DCFH (2′7′Dichorodihydrofluorescein), DHR, Dihydrocalcein AM, Dihydrorhoadamine, Dihydrothidium, DiLC1 (5), DiOC6 (3), DiOC7 (3), dKeima-Red, DRAQ5, Dronpa-Green, various forms of DsRed dTomato, various forms of DyLight, E. coli BioParticles AF488, E2-Crimson, E2-Orange, EBFP2, ECFP, various forms of eFluor, EGFP, EGFP*, Emerald, eqFP650, eqFP670, ER-Tracker Blue-White DPX, Ethidium Bromide, Express2, EYFP, Fc OxyBurst Green, Fc OxyBurst Green 123, FITC, Fluo-3, Fluo-4, Fluorescein, Fura-2, Fura-Red, GFPuv, H2DCFDA, HcRed1, Hoechst Blue (33258), Hoechst Red (33342), Hydroxycoumarin, HyPer, Indo-1, Indo-1 Blue (Low Ca2⁺), Indo-1 Violet (High Ca2⁺), iRFP, J-Red, JC-1, JC-9, Katushka (TurboFP635), Katushka2 Kusabira-Orange, LDS 751, Lissamine Rhodamine B, various forms of Live/Dead, Lucifer yellow, Lucifer Yellow CH, Lyso Tracker Blue, Lyso Tracker Green, Lyso Tracker Red, mAmertrine, Marina Blue, mBanana, mCFP, mCherry, mCitrine, Methoxycoumarin, mHoneyDew, Midoriishi-Cyan, Mithramycin, Mito Tracker Deep Red, Mito Tracker Green, Mito Tracker Orange, Mito Tracker Red, MitoFluor Green, mKate (TagFP635), mKate2, mKeima, mKeima-Red, mKO, mKOk, mNeptune, Monochlorobimane, mOrange, mOrange2, mRaspberry, mPlum, mRFP1, mStrawberry, mTangerine, mTarquoise, mTFP1, mTFP1 (Teal), NBD, OxyBurst Green H2DCFDA, OxyBurst Green H2HFF BSA, Pacific Blue, PE (R-Phycoerythrin), PE Cy5, PE Cy5.5, PE Cy7, PE Texas Red, PE-Cy5 conjugates, PE-Cy7 conjugates, PerCP (Peridinin chlorphyll protein), PerCP Cy5.5, PhiYFP, PhiYFP-m, Propidium lodide (PI), various forms of Qdot, Red 613, RFP Tomato, Rhod-2, S65A, S65C, S65L, S65T, Singlet Oxygen Sensor Green, Sirius, SNARF, Superfolder GFP, SYTOX Blue, SYTOX Green, SYTOX Orange, T-Sapphire, TagBFP, TagCFP, TagGFP, TagRFP, TagRFP657, TagYFP, tdTomato, Texas Red, Thiazole Orange, TMRE, TMRM, Topaz, TOTO-1, TO-PRO-1, TRITC, TRITC TruRed, TurboFP602, TurboFP635, TurboGFP, TurboRFP, TurboYFP, Venus, Vybrant CycleDye Violet, Wild Type GFP, X-Rhodamin, Y66F, Y66H, Y66W, YOYO-1, YPet, ZsGreen1, ZsYellow1, Zymosan A BioParticles AF488 (see more at: http://www.thefcn.org/flow-fluorochromes). Fluorophores, or simply “fluors”, are typically attached to the antibody (e.g., the immunoglobulin single variable domain) that recognizes PD-1 or to the antibody that is used as detection reagent. Various conjugated antibodies are available, such as (without being limiting) for example antibodies conjugated to Alexa Fluor®, DyLight®, Rhodamine, PE, FITC, and Cy3. Each fluorophore has a characteristic peak excitation and emission wavelength. The combination of labels which can be used will depend on the wavelength of the lamp(s) or laser(s) used to excite the fluorophore and on the detectors available.

To evaluate the competition between two test binding agents (termed A and B) for binding to PD-1, a dilution series of cold (without any label) binding agent A is added to (e.g. 200 000) cells together with the labeled binding agent B*. The concentration of binding agent B* in the test mix should be high enough to readily saturate the binding sites on PD-1 expressed on the cells. The concentration of binding agent B* that saturates the binding sites for that binding agent on PD-1 expressed on the cells can be determined with a titration series of binding agent B* on the PD-1 cells and determination of the EC50 value for binding. In order to work at saturating concentration, binding agent B* can be used at 100× the EC50 concentration.

After incubation of the cells with the mixture of binding agent A and binding agent B* and cells wash, read out can be performed on a FACS. First a gate is set on the intact cells as determined from the scatter profile and the total amount of channel fluorescence is recorded. A separate solution of binding agent B* is also prepared. Binding agent B* in this solution should be in the same buffer and at the same concentration as in the test mix (with binding agent A and B*). This separate solution is also added to the cells. After incubation and cells wash, read out can be performed on a FACS. First a gate is set on the intact cells as determined from the scatter profile and the total amount of channel fluorescence is recorded. A reduction of fluorescence for the cells incubated with the mixture of binding agent A and B* compared to the fluorescence for the cells incubated with the separate solution of binding agent B* indicates that binding agent A blocks binding by binding agent B* to PD-1 expressed on the cells.

A cross-blocking immunoglobulin, antibody, immunoglobulin single variable domain, polypeptide or other binding agent is one which will bind to the PD-1 in the above competition FACS such that during the assay and in the presence of the second binding agent the recorded fluorescence is between 80% and 0.1% (e.g. 80% to 4%) of the maximum fluorescence (measured for the separate labelled immunoglobulin, antibody, immunoglobulin single variable domain, polypeptide or other binding agent), specifically between 75% and 0.1% (e.g. 75% to 4%) of the maximum fluorescence, and more specifically between 70% and 0.1% (e.g. 70% to 4%) of maximum fluorescence (as just defined above).

The competition between two test binding agents (termed A* and B*) for binding to PD-1 can also be evaluated by adding both binding agents, each labeled with a different fluorophore, to the PD-1 expressing cells. After incubation and cells wash, read out can be performed on a FACS. A gate is set for each fluorophore and the total amount of channel fluorescence is recorded. Reduction and/or absence of fluorescence of one of the fluorophores indicates blocking by the binding agents for binding to PD-1 expressed on the cells.

Other methods for determining whether an immunoglobulin, antibody, immunoglobulin single variable domain, polypeptide or other binding agent directed against a target blocks, is capable of blocking, competitively binds or is competitive as defined herein are described e.g. in Xiao-Chi Jia et al. (Journal of Immunological Methods 288:91-98, 2004), Miller et al. (Journal of Immunological Methods 365:118-125, 2011).

As used herein, the term “potency” is a measure of the biological activity of an agent, such as an ISVD or polypeptide. Potency of an agent can be determined by any suitable method known in the art, such as for instance as described in the experimental section. Cell culture-based potency assays are often the preferred format for determining biological activity since they measure the physiological response elicited by the agent and can generate results within a relatively short period of time. Various types of cell-based assays, based on the mechanism of action of the product, can be used, such as e.g. a Tetanus-toxoid autogenic antigen-specific co-culture assay or the NFAT reporter assay (as further described in the Example section).

In contrast, the “efficacy” of an agent, such as an ISVD or polypeptide, measures the maximum strength of the effect itself, at saturating agent concentrations. Efficacy indicates the maximum response achievable from the agent. It refers to the ability of the agent to produce the desired (therapeutic) effect. The efficacy of an agent can be evaluated using in vitro functional assays or in vivo models.

### 5.2 Immunoglobulin Single Variable Domains

The present technology aims at providing a novel type of drug for treating diseases associated with PD-1. The present inventors have surprisingly found that an immunoglobulin single variable domain comprising a CDR1 (AbM numbering) consisting of the amino acid sequence of SEQ ID NO: 2394 (such as SEQ ID NO: 493), a CDR2 (AbM numbering) consisting of the amino acid sequence of SEQ ID NO: 2395 (such as SEQ ID NO: 526), and a CDR3 (AbM numbering) consisting of the amino acid sequence of SEQ ID NO: 2396 (such as SEQ ID NO: 691); or comprising a CDR1 (AbM numbering) consisting of the amino acid sequence of SEQ ID NO: 4691 (such as SEQ ID NO: 1957), a CDR2 (AbM numbering) consisting of the amino acid sequence of SEQ ID NO: 4692 (such as SEQ ID NO: 2127), and a CDR3 (AbM numbering) consisting of the amino acid sequence of SEQ ID NO: 4693 (such as SEQ ID NO: 2337); or comprising a CDR1 (AbM numbering) consisting of the amino acid sequence of SEQ ID NO: 4608, a CDR2 (AbM numbering) consisting of the amino acid sequence of SEQ ID NO: 4609, and a CDR3 (AbM numbering) consisting of the amino acid sequence of SEQ ID NO: 4610; or comprising a CDR1 (AbM numbering) consisting of the amino acid sequence of SEQ ID NO: 4696 (such as SEQ ID NO: 4611), a CDR2 (AbM numbering) consisting of the amino acid sequence of SEQ ID NO: 4697 (such as SEQ ID NO: 4481), and a CDR3 (AbM numbering) consisting of the amino acid sequence of SEQ ID NO; 4698 (such as SEQ ID NO: 4612); can be used for more potent and efficient blocking of the PD-1/PD-L1 interaction and treatment of diseases associated with PD-1.

The term “immunoglobulin single variable domain” (ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ab′)2, scFv, di-scFv), wherein two immunoglobulin domains, in particular two variable domains, interact to form an antigen binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation.

In view of the above definition, the antigen-binding domain of a conventional 4-chain antibody (such as an lgG, IgM, IgA, IgD or IgE molecule; known in the art) or of a Fab fragment, a F(ab′)2 fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a VH-VL pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single VH, a single VHH or single VL domain.

As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy-chain ISVD, such as a VHH, including a humanized VHH, a VH, including a camelized VH and a human VH. In one embodiment, it is a VHH, a camelized VH or humanized VHH. Heavy chain ISVDs can be derived from a conventional four-chain antibody or from a heavy chain antibody.

For example, the immunoglobulin single variable domain may be a single domain antibody (or an amino acid sequence that is suitable for use as a single domain antibody), a “dAb” or dAb (or an amino acid sequence that is suitable for use as a dAb) or a NANOBODY® ISVD (as defined herein, and including but not limited to a VHH); other single variable domains, or any suitable fragment of any one thereof.

In particular, the immunoglobulin single variable domain may be a NANOBODY® ISVD (such as a VHH, including a humanized VHH or camelized VH) or a suitable fragment thereof. [Note: NANOBODY @ and NANOBODIES® are registered trademarks of Ablynx N.V.]

“VHH domains”, also known as VHHs, VHH antibody fragments, have originally been described as the antigen binding immunoglobulin variable domain of “heavy chain antibodies” (i.e., of “antibodies devoid of light chains”; Hamers-Casterman et al. Nature 363:446-448, 1993). The term “VHH domain” has been chosen in order to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHH's, reference is made to the review article by Muyldermans (Reviews in Molecular Biotechnology 74:277-302, 2001).

The generation of immunoglobulin sequences, such as VHHs, has been described extensively in various publications, among which WO 94/04678, Hamers-Casterman et al. 1993 and Muyldermans et al. 2001 (Reviews in Molecular Biotechnology 74:277-302, 2001). In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production. Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be used in the present technology. Also, fully human, humanized or chimeric sequences can be used. For example, camelid immunoglobulin sequences and humanized camelid immunoglobulin sequences, or camelized domain antibodies, e.g. camelized dAb as described by Ward et al (see for example WO 94/04678 and Riechmann, Febs Lett., 339:285-290, 1994 and Prot. Eng., 9:531-537, 1996) can be used herein. The ISVDs can be fused forming a multivalent and/or multispecific construct (for multivalent and multispecific polypeptides containing one or more VHH domains and their preparation, reference is also made to Conrath et al. 2001 (J. Biol. Chem., Vol. 276, 10. 7346-7350), as well as to for example WO 96/34103 and WO 99/23221).

However, it should be noted that the ISVD comprised in the present technology is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized camelid, mouse or rabbit immunoglobulin sequences, and in particular partially or fully humanized VHH sequences), “camelized” (as defined herein) immunoglobulin sequences (and in particular camelized VH sequences), as well as ISVDs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

A “humanized VHH” comprises an amino acid sequence that corresponds to the amino acid sequence of a naturally occurring VHH domain, but that has been “humanized”, i.e. by replacing one or more amino acid residues in the amino acid sequence of said naturally occurring VHH sequence (and in particular in the framework sequences) by one or more of the amino acid residues that occur at the corresponding position(s) in a VH domain from a conventional 4-chain antibody from a human being (e.g. indicated above). This can be performed in a manner known per se, which will be clear to the skilled person, for example on the basis of the prior art (e.g. WO 2008/020079). Again, it should be noted that such humanized VHHs can be obtained in any suitable manner known per se and thus are not strictly limited to polypeptides that have been obtained using a polypeptide that comprises a naturally occurring VHH domain as a starting material.

A “camelized VH” comprises an amino acid sequence that corresponds to the amino acid sequence of a naturally occurring VH domain, but that has been “camelized”, i.e. by replacing one or more amino acid residues in the amino acid sequence of a naturally occurring VH domain from a conventional 4-chain antibody by one or more of the amino acid residues that occur at the corresponding position(s) in a VHH domain of a (camelid) heavy chain antibody. This can be performed in a manner known per se, which will be clear to the skilled person, for example on the basis of the description in the prior art (e.g. Davies and Riechman (1994 and 1996), supra). Such “camelizing” substitutions are inserted at amino acid positions that form and/or are present at the VH-VL interface, and/or at the so-called Camelidae hallmark residues, as defined herein (see for example WO 94/04678 and Davies and Riechmann (1994 and 1996), supra). In one embodiment, the VH sequence that is used as a starting material or starting point for generating or designing the camelized VH is a VH sequence from a mammal, such as the VH sequence of a human being, such as a VH3 sequence. However, it should be noted that such camelized VH can be obtained in any suitable manner known per se and thus are not strictly limited to polypeptides that have been obtained using a polypeptide that comprises a naturally occurring VH domain as a starting material.

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

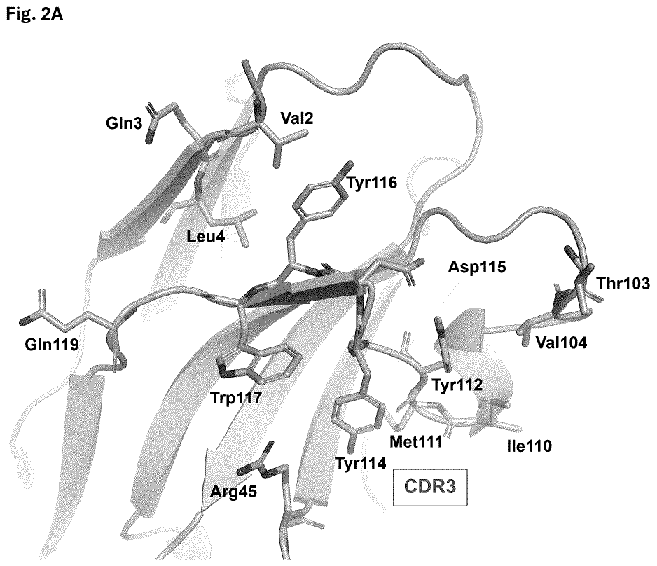

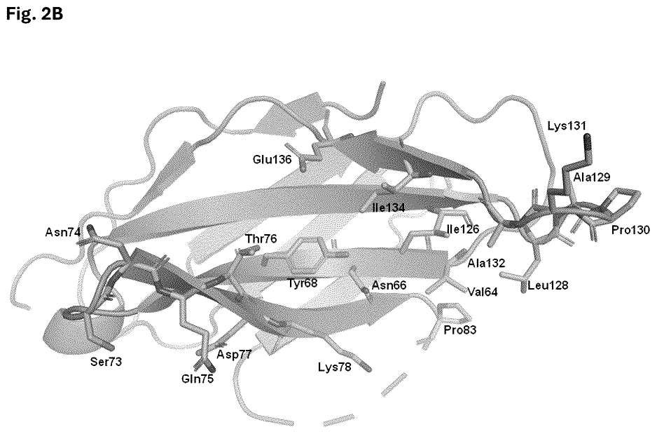

As further described in paragraph q) on pages 58 and 59 of WO 08/020079, the amino acid residues of an ISVD can be numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans, 2000 (J. Immunol. Methods 240 (1-2): 185-195; see for example FIG. 2 of this publication). It should be noted that—as is well known in the art for V_{H} domains and for V_{HH} domains the total number of amino acid residues in each of the CDRs may vary and may not correspond to the total number of amino acid residues indicated by the Kabat numbering. That is, one or more positions according to the Kabat numbering may not be occupied in the actual sequence, or the actual sequence may contain more amino acid residues than the number allowed for by the Kabat numbering. This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application, unless indicated otherwise, CDR sequences were determined according to the AbM numbering or Abm definition of CDRs (Abm numbering and Abm definition are used interchangeably) as described in Kontermann and Dübel (Eds. 2010, Antibody Engineering, vol 2, Springer Verlag Heidelberg Berlin, Martin, Chapter 3, pp. 33-51). According to this method, FR1 of an ISVD comprises the amino acid residues at positions 1-25, CDR1 of an ISVD comprises the amino acid residues at positions 26-35, FR2 of an ISVD comprises the amino acids at positions 36-49, CDR2 of an ISVD comprises the amino acid residues at positions 50-58, FR3 of an ISVD comprises the amino acid residues at positions 59-94, CDR3 of an ISVD comprises the amino acid residues at positions 95-102, and FR4 of an ISVD comprises the amino acid residues at positions 103-113.

Determination of CDR regions may also be done according to different methods, such as according to Kabat definition, Chothia definition, or IMGT definition (see Kontermann and Dübel, Eds. 2010, Antibody Engineering, vol 2, Springer Verlag Heidelberg Berlin, Martin, Chapter 3, pp. 33-51). In the CDR definition according to Kabat, FR1 of an ISVD comprises the amino acid residues at positions 1-30, CDR1 of an ISVD comprises the amino acid residues at positions 31-35, FR2 of an ISVD comprises the amino acids at positions 36-49, CDR2 of an ISVD comprises the amino acid residues at positions 50-65, FR3 of an ISVD comprises the amino acid residues at positions 66-94, CDR3 of an ISVD comprises the amino acid residues at positions 95-102, and FR4 of an ISVD comprises the amino acid residues at positions 103-113.

The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a VL-sequence) and/or from a heavy chain variable domain (e.g. a VH-sequence or VHH sequence). In one particular aspect, the framework sequences are either framework sequences that have been derived from a VHH-sequence (in which said framework sequences may optionally have been partially or fully humanized) or are conventional VH sequences that have been camelized (as defined herein).

In particular, the framework sequences present in the ISVD sequence used in the methods described herein may contain one or more of hallmark residues (as defined herein), such that the ISVD sequence is a NANOBODY® ISVD, such as e.g. a VHH, including a humanized VHH, or camelized VH. Non-limiting examples of (suitable combinations of) such framework sequences will become clear from the further disclosure herein.

Generally, NANOBODY® ISVDs (in particular VHH sequences, including (partially) humanized VHH sequences and camelized VH sequences) can be characterized by the presence of one or more “Hallmark residues” (as described herein) in one or more of the framework sequences (again as further described herein). Thus, generally, a NANOBODY® ISVD can be defined as an immunoglobulin sequence with the (general) structure

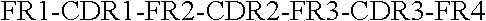

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a NANOBODY® ISVD can be an immunoglobulin sequence with the (general) structure

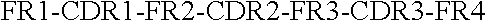

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a NANOBODY® ISVD can be an immunoglobulin sequence with the (general) structure

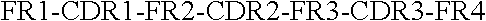

in which FR1 to FR4 refer to framework regions 1 to 4, respectively, and in which CDR1 to CDR3 refer to the complementarity determining regions 1 to 3, respectively, and in which:
one or more of the amino acid residues at positions 11, 37, 44, 45, 47, 83, 84, 103, 104 and 108 according to the Kabat numbering are chosen from the Hallmark residues mentioned in Table 1 below.

TABLE 1
Hallmark Residues in NANOBODY ® ISVDs

| Position | Human V_{H}3 | Hallmark Residues |
|---|---|---|
| 11 | L, V; predominantly L | L, S, V, M, W, F, T, Q, E, A, R, G, K, Y, N, P, I; preferably L |
| 37 | V, I, F; usually V | F⁽¹⁾, Y, V, L, A, H, S, I, W, C, N, G, D, T, P, preferably F⁽¹⁾ or |
| | | Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; most preferably G⁽²⁾ or Q⁽³⁾ |
| 45⁽⁸⁾ | L | L⁽²⁾, R⁽³⁾, P, H, F, G, Q, S, E, T, Y, C, I, D, V; preferably L⁽²⁾ |
| | | or R⁽³⁾ |
| 47⁽⁸⁾ | W, Y | F⁽¹⁾, L⁽¹⁾ or W⁽²⁾ G, I, S, A, V, M, R, Y, E, P, T, C, H, K, Q, N, |
| | | D; preferably W⁽²⁾, L⁽¹⁾ or F⁽¹⁾ |
| 83 | R or K; usually R | R, K⁽⁵⁾, T, E⁽⁵⁾, Q, N, S, I, V, G, M, L, A, D, Y, H; preferably |
| | | Kor R; most preferably K |
| 84 | A, T, D; predominantly A | p⁽⁵⁾, S, H, L, A, V, I, T, F, D, R, Y, N, Q, G, E; preferably P |
| 103 | W | W⁽⁴⁾, R⁽⁶⁾, G, S, K, A, M, Y, L, F, T, N, V, Q, P⁽⁶⁾, E, C; |
| | | preferably W |
| 104 | G | G, A, S, T, D, P, N, E, C, L; preferably G |
| 108 | L, M or T; predominantly L | Q, L⁽⁷⁾, R, P, E, K, S, T, M, A, H; preferably Q or L⁽⁷⁾ |

Notes:
⁽¹⁾In particular, but not exclusively, in combination with KERE (SEQ ID NO: 4735) or KORE (SEQ ID NO: 4736)at positions 43-46.
⁽²⁾Usually as GLEW (SEQ ID NO: 4737) at positions 44-47.
⁽³⁾Usually as KERE (SEQ ID NO: 4735) or KORE (SEQ ID NO: 4736) at positions 43-46, e.g. as KEREL (SEQ ID NO: 4738), KEREF (SEQ ID NO: 4739), KOREL (SEQ ID NO: 4740), KQREF (SEQ ID NO: 4741), KEREG (SEQ ID NO: 4742), KQREW (SEQ ID NO: 4743) or KQREG (SEQ ID NO: 4744) at positions 43-47. Alternatively, also sequences such as TERE (SEQ ID NO: 4745) (for example TEREL (SEQ ID NO: 4746)), TORE (SEQ ID NO: 4747) (for example TOREL (SEQ ID NO: 4748)), KECE (SEQ ID NO: 4749) (for example KECEL (SEQ ID NO: 4750)or KECER (SEQ ID NO: 4751)), KQCE (SEQ ID NO: 4752) (for example KQCEL (SEQ ID NO: 4753)), RERE (SEQ ID NO: 4754) (for example REREG (SEQ ID NO: 4755)), RORE (SEQ ID NO: 4756) (for example RQREL(SEQ ID NO: 4757), RQREF(SEQ ID NO: 4758) or RQREW(SEQ ID NO: 4759)), QERE (SEQ ID NO: 4760) (for example QEREG(SEQ ID NO: 4761)), QQRE(SEQ ID NO: 4762), (for example QQREW(SEQ ID NO: 4763), QQREL (SEQ ID NO: 4764)or QQREF(SEQ ID NO: 4765)), KGRE (SEQ ID NO: 4766) (for example KGREG(SEQ ID NO: 4767)), KDRE (SEQ ID NO: 4768) (for example KDREV(SEQ ID NO: 4769)) are possible. Some other possible, but less preferred sequences include for example DECKL (SEQ ID NO: 4770) and NVCEL(SEQ ID NO: 4771).
⁽⁴⁾With both GLEW (SEQ ID NO: 4737) at positions 44-47 and KERE (SEQ ID NO: 4735) or KQRE (SEQ ID NO: 4736) at positions 43-46.
⁽⁵⁾Often as KP or EP at positions 83-84 of naturally occurring VHH domains.
⁽⁶⁾In particular, but not exclusively, in combination with GLEW (SEQ ID NO: 4737) at positions 44-47.
⁽⁷⁾With the proviso that when positions 44-47 are GLEW(SEQ ID NO: 4737), position 108 is always Q in (non-humanized) VHH sequences that also contain a W at 103.
⁽⁸⁾The GLEW (SEQ ID NO: 4737) group also contains GLEW-like sequences at positions 44-47, such as for example GVEW (SEQ ID NO: 4772), EPEW (SEQ ID NO: 4773), GLER (SEQ ID NO: 4774), DQEW (SEQ ID NO: 4775), DLEW (SEQ ID NO: 4776), GIEW (SEQ ID NO: 4777), ELEW (SEQ ID NO: 4778), GPEW (SEQ ID NO: 4779), EWLP(SEQ ID NO: 4780), GPER (SEQ ID NO: 4781), GLER (SEQ ID NO: 4774) and ELEW (SEQ ID NO: 4778).

The present technology relates to ISVDs that specifically bind to PD-1. Accordingly, the target molecules for the ISVDs is PD-1. Examples are mammalian PD-1. Besides human PD-1 (SEQ ID NO: 3922), the versions from other species are also amenable to the present technology, for example PD-1 from mice, rats, rabbits, cats, dogs, goats, sheep, horses, pigs, non-human primates, such as cynomolgus monkeys (also referred to herein as “cyno”), or camelids, such as llama or alpaca.

In some embodiments, the ISVDs of the present technology bind to human PD-1 (hPD-1; SEQ ID NO: 3922). In some embodiments, the ISVDs of the present technology bind to human PD-1 and cyno PD-1 (cPD-1; SEQ ID NO: 3923). Sequences of human, and cyno PD-1 are depicted in Table A-1.

The immunoglobulin single variable domains of the present technology show a highly affinity on PD-1 (e.g. as measured by Surface Plasmon Resonance) as a (monovalent) single variable domain. In some embodiments, the immunoglobulin single variable domains of the present technology show a highly potency on PD-1 (e.g. as measured in (FACS) competition assay, TT assay and/or NFAT reporter assay) as a (monovalent) single variable domain. In some embodiments, the immunoglobulin single variable domains of the present technology show a highly efficacy (e.g. as measured in (FACS) competition assay, TT assay and/or NFAT reporter assay) on PD-1 as a (monovalent) single variable domain. In some embodiments, the immunoglobulin single variable domains of the present technology are efficiently produced (e.g. in microbial hosts, such as Pichia, e.g. P. pastoris), even when encompassed with binders to other targets, and have good stability.

In some embodiments, the immunoglobulin single variable domains of the present technology show a highly affinity on PD-1 as a (monovalent) single variable domain. The affinity of a molecular interaction between two molecules can be measured via different techniques known per se, such as the well-known surface plasmon resonance (SPR) biosensor technique (see for example Ober et al. 2001, Intern. Immunology 13:1551-1559). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kon, koff measurements and hence KD (or KA) values. This can for example be performed using the well-known BIAcore® system (BIAcore International AB, a GE Healthcare company, Uppsala, Sweden and Piscataway, NJ). For further descriptions, see Jonsson et al. 1993 (Ann. Biol. Clin. 51:19-26), Jonsson et al. 1991 (Biotechniques 11:620-627), Johnsson et al. 1995 (J. Mol. Recognit. 8:125-131), and Johnsson et al. 1991 (Anal. Biochem. 198:268-277).

Another well-known biosensor technique to determine affinities of biomolecular interactions is bio-layer interferometry (BLI) (see for example Abdiche et al. 2008, Anal. Biochem. 377:209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA).

Alternatively, affinities can be measured in Kinetic Exclusion Assay (KinExA) (see for example Drake et al. 2004, Anal. Biochem., 328:35-43), using the KinExA® platform (Sapidyne Instruments Inc, Boise, USA). The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen).

The GYROLAB® immunoassay system provides a platform for automated bioanalysis and rapid sample turnaround (Fraley et al. 2013, Bioanalysis 5:1765-74).

Because of their small size, (monovalent) immunoglobulin single variable domains are more efficient for the penetration of the tumor environment. In addition, they are easily combined with other (cancer) target binders in a single chain when the simultaneous targeting of multiple (cancer) targets is intended, e.g., as multivalent ISVD construct. The high production yields and better stability demonstrated for immunoglobulin single variable domains makes them ideally suited for subcutaneous administration.

The interacting amino acid residues of the ISVD of the present technology (paratope) and the PD-1 protein (epitope) preferably have a distance of <4 Å, wherein the distance between the amino acid residues is measured e.g., in X-ray crystallography or in Cryogenic-electron microscopy (cryo-EM). Thus, in an embodiment, two amino acid residues are considered to interact when the distance between them is 4.0 Å or less.

The interacting amino acid residues of the ISVD of the present technology may be identified by a person skilled in the art, e.g., the skilled person knows that structural information allowing to determine the distance between two (amino acids) residues may be obtained using standard methods such as X-ray crystallography, Cryogenic electron microscopy (cryo-EM), nuclear magnetic resonance, and subsequent molecular modelling. Hence, the interacting amino acid residue(s) in the ISVD and in human PD-1 protein are identified by cryo-EM as described in the examples.

Based on the structural data provided herein, the skilled person understands that amino acid residues of an ISVD involved in the interaction with human PD-1 can be determined and that this interaction site is important for the binding of an ISVD to the epitope on human PD-1.

Specifically, the interaction site between an ISVD having SEQ ID NO: 402 and human PD-1 having SEQ ID NO: 3922 has been determined and provides clear technical guidance for the generation of further ISVDs having the binding specificity of the ISVD having SEQ ID NO: 402, e.g. binding to the same epitope on human PD-1.

In this respect, the skilled person understands that an ISVD of the present technology having the binding specificity of the ISVD having SEQ ID NO: 402, e.g. specifically binding to the same epitope on human PD-1 or blocking the binding to human PD-1 by an ISVD having SEQ ID NO: 402 is not limited to the specific amino acid sequence of the CDR region and/or the FR region of the ISVD having SEQ ID NO: 402, as long as the ISVD contains at least one or more amino acid residue(s) corresponding to the at least one or more amino acid residue(s) of the ISVD having SEQ ID NO: 402 that interact with human PD-1 as described herein.

In other words, an ISVD of the present technology having the binding specificity of the ISVD having SEQ ID NO: 402, e.g. specifically binding to the same epitope on human PD-1 or blocking the binding to human PD-1 by an ISVD having SEQ ID NO: 402, may contain, as compared to an ISVD having SEQ ID NO: 402, mutations and/or substitutions within the CDR region and/or within the FR region (such as conservative amino acid substitutions) whilst maintaining the amino acids of the ISVD that form the interaction site (i.e., the paratope on the ISVD) with human PD-1.

Another example of the ISVDs of the present technology specifically binding to PD-1 is an ISVD that essentially consists of 4 framework regions (FR1 to FR4, respectively) and 3 complementarity determining regions (CDR1 to CDR3, respectively), wherein the ISVD binds to an (discontinuous) epitope on human PD-1, wherein the ISVD cross-blocks the binding to human PD-1 protein of a polypeptide with the amino acid sequence of SEQ ID Nos: 1-488 or wherein the ISVD is cross-blocked from binding to human PD-1 protein by a polypeptide with the amino acid sequence of SEQ ID Nos: 1-488.

In an embodiment, the ISVD competes with a polypeptide with the amino acid sequence of SEQ ID No: 1-488 for binding human PD-1 protein, as measured in a competition assay, such as a FACS competition assay.

### 5.2.1 Embodiment 1, Family A0294047E02

There is provided an immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1 protein, wherein at least one amino acid residue in the ISVD is capable of interacting with at least one amino acid residue in at least one, preferably in each, of the following stretches (of the ectodomain) of human PD-1 protein, when numbered in accordance with SEQ ID NO: 4703: V64-P83, and I126-E136, preferably when the ISVD and human PD-1 protein are mixed in PBS1× buffer and crystallized by hanging drop vapor diffusion using 30% PEG3350, 50 mM TRIS, pH 8 at 19° C.

In some embodiments, the at least one amino acid residue in at least one, preferably in each stretch (of the ectodomain) of human PD-1 protein is selected from:
For stretch V64-P83: V64, N66, Y68, S73, N74, Q75, T76, D77, K78, and P83;
For stretch I126-E136: I126, L128, A129, P130, K131, A132, I134, and E136, when numbered in accordance with SEQ ID NO: 4703.

In some embodiments, at least one amino acid residue located in the ISVD is capable of interacting with at least two, at least three, at least four, at least five, at least six, at least seven, at least eight, at least nine, or all of the following amino acid residues in stretch V64-P83 (of the ectodomain) of human PD-1 protein, when numbered in accordance with SEQ ID NO: 4703: V64, N66, Y68, S73, N74, Q75, T76, D77, K78, and P83.

In some embodiments, at least one amino acid residue located in the ISVD is capable of interacting with at least two, at least three, at least four, at least five, at least six, at least seven, or all of the following amino acid residues in stretch I126-E136 (of the ectodomain) of human PD-1 protein when numbered in accordance with SEQ ID NO: 4703: I126, L128, A129, P130, K131, A132, I134, and E136.

In some embodiments, at least one amino acid residue located in the ISVD is capable of forming salt bridges with at least the amino acid residue E136 of human PD-1 protein, when numbered in accordance with SEQ ID NO: 4703, preferably when the ISVD and human PD-1 protein are mixed in PBS1× buffer and crystallized by hanging drop vapor diffusion using 30% PEG3350, 50 mM TRIS, pH 8 at 19° C.

In some embodiments, at least one amino acid residue located in the ISVD is capable of forming hydrogen bonds with at least one, preferably all, of the following amino acid residues of human PD-1 protein, when numbered in accordance with SEQ ID NO: 4703: N66, Y68, K78, A132, optionally wherein at least one amino acid residue located in the ISVD is further capable of forming hydrogen bonds with the amino acid residue N74 of human PD-1 protein, when numbered in accordance with SEQ ID NO: 4703, preferably when the ISVD and human PD-1 protein are mixed in PBS1× buffer and crystallized by hanging drop vapor diffusion using 30% PEG3350, 50 mM TRIS, pH 8 at 19° C.

In some embodiments, the least one amino acid residue located in the ISVD is capable of interacting with an (discontinuous) epitope on human PD-1 comprising at least one amino acid residue in each of the following stretches (of the ectodomain) of human PD-1 protein: V64-P83, and I126-E136, when numbered in accordance with SEQ ID NO: 4703.

In some embodiments, the at least one amino acid residue in each stretch (of the ectodomain) of human PD-1 protein is selected from:
For stretch V64-P83: V64, N66, Y68, S73, N74, Q75, T76, D77, K78, and P83;
For stretch I126-E136: I126, L128, A129, P130, K131, A132, I134, and E136, when numbered in accordance with SEQ ID NO: 4703.

In some embodiments, at least one amino acid residue located in the ISVD is capable of interacting with at least one, at least two, at least three, at least four, at least five, at least six, at least seven, at least ten, at least fifteen, preferably all, of the following amino acid residues of human PD-1 protein, when numbered in accordance with SEQ ID NO: 4703: V64, N66, Y68, S73, N74, Q75, T76, D77, K78, P83, 1126, L128, A129, P130, K131, A132, I134, and E136, preferably when the ISVD and human PD-1 protein are mixed in PBS1× buffer and crystallized by hanging drop vapor diffusion using 30% PEG3350, 50 mM TRIS, pH 8 at 19° C.

In some embodiments, the at least one amino acid residue located in the ISVD that is capable of interacting with amino acid residues of human PD-1 protein is located in CDR3.

In some embodiments, the amino acid at position 45 (Kabat numbering) is capable of interacting, preferably by forming a salt bridge, with E136 in PD-1, preferably when the ISVD and human PD-1 protein are mixed in PBS1× buffer and crystallized by hanging drop vapor diffusion using 30% PEG3350, 50 mM TRIS, pH 8 at 19° C.

In some embodiments:
the amino acid residue at position 100e (Kabat numbering) is capable of interacting, preferably by forming a hydrogen bond, with A132 in PD-1;
the amino acid residue at position 100h (Kabat numbering) is capable of interacting, preferably by forming a hydrogen bond, with N66 in PD-1;
the amino acid residue at position 100j (Kabat numbering) is capable of interacting, preferably by forming a hydrogen bond, with N66 in PD-1;
the amino acid residue at position 100j (Kabat numbering) is capable of interacting, preferably by forming a hydrogen bond, with Y68 in PD-1;
the amino acid residue at position 100j (Kabat numbering) is capable of interacting, preferably by forming a hydrogen bond, with A132 in PD-1;
the amino acid residue at position 101 (Kabat numbering) is capable of interacting, preferably by forming a hydrogen bond, with N66 in PD-1; and/or
the amino acid residue at position 101 (Kabat numbering) is capable of interacting, preferably by forming a hydrogen bond, with K78 in PD-1;
   preferably when the ISVD and human PD-1 protein are mixed in PBS1× buffer and crystallized by hanging drop vapor diffusion using 30% PEG3350, 50 mM TRIS, pH 8 at 19° C.

In some embodiments, two amino acids are considered to interact when the distance between them is 4.0 Å or less.

In some embodiments, the interacting amino acid residue(s) in the ISVD and in human PD-1 protein are identified by X-ray crystallography by:
Mixing human PD-1 protein, and the ISVD in PBS1× buffer;
Concentrating the protein complex to 10.6 mg/ml;
Crystallizing by hanging drop vapor diffusion using 30% PEG3350, 50 mM TRIS, pH 8 at 19° C.;
Collecting X-ray diffraction data;
Processing the collected data, preferably as described in Example 13.

In some embodiments, the (discontinuous) epitope comprises or consists of the following amino acid residues of human PD-1 protein, when numbered in accordance with SEQ ID NO: 4703: V64, N66, Y68, T76, D77, K78, P83, 1126, L128, A129, P130, K131, A132, I134, E136.

In some embodiments, the (discontinuous) epitope comprises or consists of the following amino acid residues of human PD-1 protein, when numbered in accordance with SEQ ID NO: 4703: V64, N66, Y68, N74, T76, D77, K78, P83, 1126, L128, A129, P130, K131, A132, I134, E136.

In some embodiments, the (discontinuous) epitope comprises or consists of the following amino acid residues of human PD-1 protein, when numbered in accordance with SEQ ID NO: 4703: V64, N66, Y68, S73, Q75, T76, D77, K78, P83, 1126, L128, A129, P130, K131, A132, I134, E136.

In some embodiments, the (discontinuous) epitope comprises or consists of the following amino acid residues of human PD-1 protein, when numbered in accordance with SEQ ID NO: 4703: V64, N66, Y68, S73, N74, Q75, T76, D77, K78, P83, 1126, L128, A129, P130, K131, A132, I134, E136.

In some embodiments, the ISVD cross-blocks the binding to human PD-1 protein of a polypeptide having an amino acid sequence selected from SEQ ID NOs: 1-488, preferably the ISVD cross-blocks the binding to human PD-1 protein of a polypeptide having an amino acid sequence selected from SEQ ID NO: 402, or the binding of the ISVD to human PD-1 protein is cross-blocked by a polypeptide having an amino acid sequence selected from SEQ ID NOs: 1-488, preferably SEQ ID NO: 402.

There is provided an immunoglobulin single variable domain (ISVD) specifically binding human PD-1 protein, that essentially consists of 4 framework regions (FR1 to FR4, respectively) and 3 complementarity determining regions (CDR1 to CDR3, respectively), wherein the ISVD comprises an amino acid residue capable of forming salt bridges at at least position 45 (Kabat numbering), preferably wherein the ISVD comprises at least the following amino acid residue: R45 (Kabat numbering).

In some embodiments, the ISVD comprises an amino acid residue capable of forming hydrogen bonds at at least one, preferably at all, of the following positions: 100e, 100h, 100j, and 101 (Kabat numbering) preferably wherein the ISVD comprises at least one, preferably all of the following amino acid residues at the following positions: I100e, Y100h, Y100j, and D101 (Kabat numbering).

In some embodiments, the ISVD comprises at least one, at least two, at least three, preferably all of the following amino acid residues at the following positions (Kabat numbering): T99, V100, 1100e, M100g, Y100h, Y100j, D101, and Y102, preferably at least I100e, Y100h, Y100j, and D101.

In some embodiments, the CDR3 (AbM definition) consists of the following amino acid sequence X₁X₂STTVRX₃X₄CX₅IMYX₆YDY (SEQ ID NO: 4702), wherein:
X₁ is selected from any amino acid, preferably X₁ is selected from A or G;
X₂ is selected from any amino acid, preferably X₂ is selected T, A, E, G, F, I, D, H, K, S, P, R, Q, Y, V, or L;
X₃ is selected from any amino acid, preferably X₃ is selected from D or G;
X₄ is selected from any amino acid, preferably X₄ is selected from L, M, I, E, A, V, D, H, G, F, K, R, P, S, T, or Y;
X₅ is selected from any amino acid, preferably X₅ is selected from G or F; and
X₆ is selected from any amino acid, preferably X₆ is selected from L, V, or F.

In some embodiments, the following amino acid residue of the ISVD at the following position: R45 (Kabat numbering) is capable of forming salt bridges with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C.

In some embodiments, at least one, preferably all of the following amino acid residues of the ISVD at the following positions: I100e, Y100h, Y100j, and D101 (Kabat numbering) are capable of forming hydrogen bonds with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C.

In some embodiments, at least one, preferably all, of the following amino acid residues of the ISVD at the following positions: R45, I100e, Y100h, Y100j, and D101, and optionally Q105 (Kabat numbering) is capable of interacting with at least one, preferably all of the following amino acid residues of human PD-1 protein, when numbered in accordance with SEQ ID NO: 4703: N66, Y68, K78, A132, and E136, and optionally N74, preferably wherein:
R45 in the ISVD (Kabat numbering) interacts, preferably by forming a salt bridge, with E136 in human PD-1 protein;
I100e in the ISVD (Kabat numbering) interacts, preferably by forming a hydrogen bond, with A132 in human PD-1 protein;
Y100 h in the ISVD (Kabat numbering) interacts, preferably by forming a hydrogen bond, with N66 in human PD-1 protein;
Y100j in the ISVD (Kabat numbering) interacts, preferably by forming a hydrogen bond, with N66 in human PD-1 protein;
Y100j in the ISVD (Kabat numbering) interacts, preferably by forming a hydrogen bond, with Y68 in human PD-1 protein;
Y100j in the ISVD (Kabat numbering) interacts, preferably by forming a hydrogen bond, with A132 in human PD-1 protein;
D101 in the ISVD (Kabat numbering) interacts, preferably by forming a hydrogen bond, with N66 in human PD-1 protein; and/or
D101 in the ISVD (Kabat numbering) interacts, preferably by forming a hydrogen bond, with K78 in human PD-1 protein;
   preferably when the ISVD and human PD-1 protein are mixed in PBS1× buffer and crystallized by hanging drop vapor diffusion using 30% PEG3350, 50 mM TRIS, pH 8 at 19° C.

In one embodiment, Q105 in the ISVD (Kabat numbering) interacts, preferably by forming a hydrogen bond, with N74 in human PD-1 protein.

In some embodiments, the ISVD comprises at least one, at least two, at least three, preferably all of the following amino acid residues at the following positions (Kabat numbering): I100e, Y100h, Y100j, and D101.

In some embodiments, the ISVD comprises at least one, at least two, at least three, preferably all of the following amino acid residues at the following positions (Kabat numbering): R45, I100e, Y100h, Y100j, and D101.

In some embodiments, the ISVD comprises at least one, at least two, at least three, preferably all of the following amino acid residues at the following positions (Kabat numbering): R45, I100e, Y100h, Y100j, D101, and Q105.

In some embodiments, the ISVD comprises at least one, at least two, at least three, at least four, at least five, at least six, at least seven, preferably all of the following amino acid residues at the following positions (Kabat numbering): T99, V100, I100e, M100g, Y100h, Y100j, D101, and Y102.

In some embodiments, the ISVD comprises at least one, at least two, at least three, at least four, at least five, at least six, at least seven, preferably all of the following amino acid residues at the following positions (Kabat numbering): R45, T99, V100, I100e, M100g, Y100h, Y100j, D101, and Y102.

In some embodiments, the ISVD essentially consists of 4 framework regions (FR1 to FR4, respectively) and 3 complementarity determining regions (CDR1 to CDR3 respectively), in which CDR3 (AbM definition) has an amino acid sequence selected from:
a) the amino acid sequence of SEQ ID NO: 691;
b) amino acid sequences that have at least 80% amino acid identity with the amino acid sequence of SEQ ID NO: 691;
c) amino acid sequences that have 3, 2, or 1 amino acid difference with the amino acid sequence of SEQ ID NO: 691.

In some embodiments, the ISVD comprises V2, Q3, L4, R45, W103 and/or Q105 (Kabat numbering).

In some embodiments, the ISVD comprises R45 (Kabat numbering).

In some embodiments, the ISVD comprises:
R45 and Q105 (Kabat numbering);
R45 and W103 (Kabat numbering); or
R45, W103 and Q105 (Kabat numbering).

In some embodiments, the ISVD comprises at least one, at least two, at least three, at least four, at least five, at least six, at least seven, at least eight, at least nine, at least ten, at least eleven, at least twelve, at least thirteen, preferably all of the following amino acid residues at the following positions (Kabat numbering): V2, Q3, L4, R45, T99, V100, I100e, M100g, Y100h, Y100j, D101, Y102, W103, and Q105.

In some embodiments, R45, I100e, Y100h, Y100j, and D101, and optionally Q105 (Kabat numbering) of the ISVD are capable of interacting with at least one of the following amino acids of human PD-1 protein, when numbered in accordance with SEQ ID NO: 4703: N66, Y68, K78, A132, E136, and optionally N74, preferably when the ISVD and human PD-1 protein are mixed in PBS1× buffer and crystallized by hanging drop vapor diffusion using 30% PEG3350, 50 mM TRIS, pH 8 at 19° C.

In some embodiments, V2, Q3, L4, R45, I100e, Y100h, Y100j, D101, and Q105 (Kabat numbering) of the ISVD are capable of interacting with the following amino acids of human PD-1 protein, when numbered in accordance with SEQ ID NO: 4703: N66, Y68, S73, N74, Q75, K78, A132, and E136, preferably when the ISVD and human PD-1 protein are mixed in PBS1× buffer and crystallized by hanging drop vapor diffusion using 30% PEG3350, 50 mM TRIS, pH 8 at 19° C.

In some embodiments, wherein two amino acids are considered to interact when the distance between them is 4.0 Å or less.

In some embodiments, the interacting amino acid residue(s) in the ISVD and in human PD-1 protein are identified by X-ray crystallography by:
Mixing human PD-1 protein, and the ISVD in PBS1× buffer;
Concentrating the protein complex to 10.6 mg/ml;
Crystallizing by hanging drop vapor diffusion using 30% PEG3350, 50 mM TRIS, pH 8 at 19° C.;
Collecting X-ray diffraction data;
Processing the collected data, preferably as described in Example 13.

There is provided an immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD cross-blocks the binding to human PD-1 protein of a polypeptide having an amino acid sequence selected from SEQ ID NOs: 1-488, preferably wherein the ISVD cross-blocks the binding to human PD-1 protein of a polypeptide having an amino acid sequence selected from SEQ ID NO: 402, or wherein the binding of the ISVD to human PD-1 protein is cross-blocked by a polypeptide having an amino acid sequence selected from SEQ ID NOs: 1-488, preferably SEQ ID NO: 402.

In some embodiments, the ISVD competes with a polypeptide with an amino acid sequence selected from SEQ ID NO: 1-488, preferably SEQ ID NO: 402, for binding human PD-1 protein, as measured in a competition assay, such as a FACS competition assay.

There is provided an immunoglobulin single variable domain (ISVD) specifically binding human PD-1, that essentially consists of 4 framework regions (FR1 to FR4, respectively) and 3 complementarity determining regions (CDR1 to CDR3, respectively), wherein CDR3 (AbM definition) consists of the amino acid sequence of one of SEQ ID NOs: 644-711, and wherein the amino acid residue of the ISVD at the following position: 45 (Kabat numbering) is capable of forming salt bridges with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C., preferably wherein the ISVD comprises at least the following amino acid residue: R45 (Kabat numbering).

In some embodiments, R45 (Kabat numbering) of the ISVD is capable of forming salt bridges with the amino acid residue E136 of human PD-1 protein, when numbered in accordance with SEQ ID NO: 4703.

In some embodiments, the CDR3 (AbM definition) consists of the amino acid sequence of SEQ ID NO: 691.

Specific examples of ISVDs specifically binding to PD-1 are ISVDs that comprises 4 framework regions (FR1 to FR4, respectively) and three complementarity determining regions (CDR1 to CDR3, respectively), wherein:
CDR1 (AbM numbering) consists of an amino acid sequence selected from:
   a) the amino acid sequence of GFTLDX₁YX₂X₃G (SEQ ID NO: 2394), wherein X₁ is selected from A, D, Q, P, S, G, T, V, I, R, K, E, L, H, F, or Y, X₂ is selected from V or F, and X₃ is selected from I, E, D, A, F, G, H, L, K, T, M, S, P, R, Y, V, or Q;
   b) amino acid sequences that have at least 80% amino acid identity with the amino acid sequence of GFTLDX₁YX₂X₃G (SEQ ID NO: 2394), wherein X₁ is selected from A, D, Q, P, S, G, T, V, I, R, K, E, L, H, F, or Y, X₂ is selected from V or F, and X₃ is selected from I, E, D, A, F, G, H, L, K, T, M, S, P, R, Y, V, or Q;
   c) amino acid sequences that have 3, 2, or 1 amino acid difference with the amino acid sequences of GFTLDX₁YX₂X₃G (SEQ ID NO: 2394), wherein X₁ is selected from A, D, Q, P, S, G, T, V, I, R, K, E, L, H, F, or Y, X₂ is selected from V or F, and X₃ is selected from I, E, D, A, F, G, H, L, K, T, M, S, P, R, Y, V, or Q;
      and
CDR2 (AbM numbering) consists of an amino acid sequence selected from:
   a) the amino acid sequence of CX₁SX₂X₃X₄DRX₅X₆ (SEQ ID NO: 2395), wherein X₁ is selected from L, G, H, K, P, R, S, or T, X₂ is selected from S, E, G, H, P, or Q, and X₃ is selected from N, E, A, D, F, G, H, I, K, L, P, Q, R, S, T, V, or Y, X₄ is selected from G, D, F, H, K, L, P, Q, R, S, T, V, A, or Y, X₅ is selected from I, A, E, G, H, K, P, R, S, or T, and X₆ is selected from N, A, D, E, F, G, H, I, K, L, P, Q, R, S, T, V, or Y;
   b) amino acid sequences that have at least 80% amino acid identity with the amino acid sequence of CX₁SX₂X₃X₄DRX₅X₆ (SEQ ID NO: 2395), wherein X₁ is selected from L, G, H, K, P, R, S, or T, X₂ is selected from S, E, G, H, P, or Q, and X₃ is selected from N, E, A, D, F, G, H, I, K, L, P, Q, R, S, T, V, or Y, X₄ is selected from G, D, F, H, K, L, P, Q, R, S, T, V, A, or Y, X₅ is selected from I, A, E, G, H, K, P, R, S, or T, and X₆ is selected from N, A, D, E, F, G, H, I, K, L, P, Q, R, S, T, V, or Y;
   c) amino acid sequences that have 3, 2, or 1 amino acid difference with the amino acid sequence of CX₁SX₂X₃X₄DRX₅X₆ (SEQ ID NO: 2395), wherein X₁ is selected from L, G, H, K, P, R, S, or T, X₂ is selected from S, E, G, H, P, or Q, and X₃ is selected from N, E, A, D, F, G, H, I, K, L, P, Q, R, S, T, V, or Y, X₄ is selected from G, D, F, H, K, L, P, Q, R, S, T, V, A, or Y, X₅ is selected from I, A, E, G, H, K, P, R, S, or T, and X₆ is selected from N, A, D, E, F, G, H, I, K, L, P, Q, R, S, T, V, or Y;
      and
CDR3 (AbM numbering) consists of an amino acid sequence selected from:
   a) the amino acid sequence of X₁X₂STX₃VRX₄X₅CX₆IX₇YX₈YDX₉ (SEQ ID NO: 2396), wherein X₁ is selected from A or G, X₂ is selected from T, A, E, G, F, I, D, H, K, S, P, R, Q, Y, V, or L, X₃ is selected from T or I, X₄ is selected from D or G, X₅ is selected from L, M, I, E, A, V, D, H, G, F, K, R, P, S, T, or Y, X₆ is selected from G or F, X₇ is selected from I, M, F, Y, L, A, D, E, G, K, H, P, Q, S, T, V, or R, X₈ is selected from L, V, or F, and X₉ is selected from Y, E, I, L, or V;
   b) amino acid sequences that have at least 80% amino acid identity with the amino acid sequence X₁X₂STX₃VRX₄X₅CX₆IX₇YX₈YDX₉ (SEQ ID NO: 2396), wherein X₁ is selected from A or G, X₂ is selected from T, A, E, G, F, I, D, H, K, S, P, R, Q, Y, V, or L, X₃ is selected from T or I, X₄ is selected from D or G, X₅ is selected from L, M, I, E, A, V, D, H, G, F, K, R, P, S, T, or Y, X₆ is selected from G or F, X₇ is selected from I, M, F, Y, L, A, D, E, G, K, H, P, Q, S, T, V, or R, X₈ is selected from L, V, or F, and X₉ is selected from Y, E, I, L, or V;
   c) amino acid sequences that have 3, 2, or 1 amino acid difference with the amino acid sequence of X₁X₂STX₃VRX₄X₅CX₆IX₇YX₈YDX₉ (SEQ ID NO: 2396), wherein X₁ is selected from A or G, X₂ is selected from T, A, E, G, F, I, D, H, K, S, P, R, Q, Y, V, or L, X₃ is selected T or I, X₄ is selected from D or G, X₅ is selected from L, M, I, E, A, V, D, H, G, F, K, R, P, S, T, V, or Y, X₆ is selected from G or F, X₇ is selected from I, M, F, Y, L, A, D, E, G, K, H, P, Q, S, T, V, or R, X₈ is selected from L, V, or F, and X₉ is selected from Y, E, I, L, or V.

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NO: 2394, SEQ ID NO: 2395 or SEQ ID NO: 2396.

In one embodiment, the ISVD comprises CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 1-488.

In some embodiments, the ISVD essentially consists of 4 framework regions (FR1 to FR4, respectively) and 3 complementarity determining regions (CDR1 to CDR3 respectively), in which:
CDR1 (AbM numbering) consists of an amino acid sequence selected from:
   a) the amino acid sequence of GFTLDYYVIG (SEQ ID NO: 493);
   b) amino acid sequences that have at least 80% amino acid identity with the amino acid sequence of GFTLDYYVIG (SEQ ID NO: 493);
   c) amino acid sequences that have 3, 2, or 1 amino acid difference with the amino acid sequences of GFTLDYYVIG (SEQ ID NO: 493);
      and
CDR2 (AbM numbering) consists of an amino acid sequence selected from:
   a) the amino acid sequence of CLSSX₁GDRX₂N (SEQ ID NO: 4707), wherein X₁ is selected from N, E, S, and R, and X₂ is selected from I and T;
   b) amino acid sequences that have at least 80% amino acid identity with the amino acid sequence of CLSSX₁GDRX₂N (SEQ ID NO: 4707), wherein X₁ is selected from N, E, S, and R, and X₂ is selected from I and T;
   c) amino acid sequences that have 3, 2, or 1 amino acid difference with the amino acid sequence of CLSSX₁GDRX₂N (SEQ ID NO: 4707), wherein X₁ is selected from N, E, S, and R, and X₂ is selected from I and T;
      and
CDR3 (AbM numbering) consists of an amino acid sequence selected from:
   a) the amino acid sequence of GTSTTVRDXCGIMYLYDY (SEQ ID NO: 4708), wherein X is selected from M, L, V, and R;
   b) amino acid sequences that have at least 80% amino acid identity with the amino acid sequence GTSTTVRDXCGIMYLYDY (SEQ ID NO: 4708), wherein X is selected from M, L, V, and R;
   c) amino acid sequences that have 3, 2, or 1 amino acid difference with the amino acid sequence GTSTTVRDXCGIMYLYDY (SEQ ID NO: 4708), wherein X is selected from M, L, V, and R.

In some embodiments, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 493;
   b) amino acid sequences that have at least 80% amino acid identity with the amino acid sequence of SEQ ID NO: 493; and
   c) amino acid sequences that have 3, 2, or 1 amino acid difference with the amino acid sequences of SEQ ID NO: 493;
      and
CDR2 (AbM definition) has an amino acid sequence selected from:
   a) the amino acid sequence of SEQ ID NO: 526;
   b) amino acid sequences that have at least 80% amino acid identity with the amino acid sequence of SEQ ID NO: 526;
   c) amino acid sequences that have 3, 2, or 1 amino acid difference with the amino acid sequence of SEQ ID NO: 526;
and
CDR3 (AbM definition) has an amino acid sequence selected from:
   a) the amino acid sequence of SEQ ID NO: 691;
   b) amino acid sequences that have at least 80% amino acid identity with the amino acid sequence of SEQ ID NO: 691;
   c) amino acid sequences that have 3, 2, or 1 amino acid difference with the amino acid sequence of SEQ ID NO: 691.

In some embodiments, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 493;
   b) amino acid sequences that have at least 80% amino acid identity with the amino acid sequence of SEQ ID NO: 493; and
   c) amino acid sequences that have 3, 2, or 1 amino acid difference with the amino acid sequences of SEQ ID NO: 493;
and
CDR2 (AbM definition) has an amino acid sequence selected from:
   a) the amino acid sequence of SEQ ID NO: 543;
   b) amino acid sequences that have at least 80% amino acid identity with the amino acid sequence of SEQ ID NO: 543;
   c) amino acid sequences that have 3, 2, or 1 amino acid difference with the amino acid sequence of SEQ ID NO: 543;
and
CDR3 (AbM definition) has an amino acid sequence selected from:
   a) the amino acid sequence of SEQ ID NOs: 680;
   b) amino acid sequences that have at least 80% amino acid identity with the amino acid sequence of SEQ ID NO: 680;
   c) amino acid sequences that have 3, 2, or 1 amino acid difference with the amino acid sequence of SEQ ID NO: 680.

In some embodiments, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 493;
   b) amino acid sequences that have at least 80% amino acid identity with the amino acid sequence of SEQ ID NO: 493; and
   c) amino acid sequences that have 3, 2, or 1 amino acid difference with the amino acid sequences of SEQ ID NO: 493;
and
CDR2 (AbM definition) has an amino acid sequence selected from:
   a) the amino acid sequence of SEQ ID NO: 594;
   b) amino acid sequences that have at least 80% amino acid identity with the amino acid sequence of SEQ ID NO: 594;
   c) amino acid sequences that have 3, 2, or 1 amino acid difference with the amino acid sequence of SEQ ID NO: 594;
and
CDR3 (AbM definition) has an amino acid sequence selected from:
   a) the amino acid sequence of SEQ ID NO: 707;
   b) amino acid sequences that have at least 80% amino acid identity with the amino acid sequence of SEQ ID NO: 707;
   c) amino acid sequences that have 3, 2, or 1 amino acid difference with the amino acid sequence of SEQ ID NO: 707.

In some embodiments, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 493;
   b) amino acid sequences that have at least 80% amino acid identity with the amino acid sequence of SEQ ID NO: 493; and
   c) amino acid sequences that have 3, 2, or 1 amino acid difference with the amino acid sequences of SEQ ID NO: 493;
and
CDR2 (AbM definition) has an amino acid sequence selected from:
   a) the amino acid sequence of SEQ ID NO: 593;
   b) amino acid sequences that have at least 80% amino acid identity with the amino acid sequence of SEQ ID NO: 593;
   c) amino acid sequences that have 3, 2, or 1 amino acid difference with the amino acid sequence of SEQ ID NO: 593;
and
CDR3 (AbM definition) has an amino acid sequence selected from:
   a) the amino acid sequence of SEQ ID NO: 701;
   b) amino acid sequences that have at least 80% amino acid identity with the amino acid sequence of SEQ ID NO: 701;
   c) amino acid sequences that have 3, 2, or 1 amino acid difference with the amino acid sequence of SEQ ID NO: 701.

In some embodiments, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 493;
   b) amino acid sequences that have at least 80% amino acid identity with the amino acid sequence of SEQ ID NO: 493; and
   c) amino acid sequences that have 3, 2, or 1 amino acid difference with the amino acid sequences of SEQ ID NO: 493;
and
CDR2 (AbM definition) has an amino acid sequence selected from:
   a) the amino acid sequence of SEQ ID NO: 549;
   b) amino acid sequences that have at least 80% amino acid identity with the amino acid sequence of SEQ ID NO: 549;
   c) amino acid sequences that have 3, 2, or 1 amino acid difference with the amino acid sequence of SEQ ID NO: 549;
and
CDR3 (AbM definition) has an amino acid sequence selected from:
   a) the amino acid sequence of SEQ ID NO: 680;
   b) amino acid sequences that have at least 80% amino acid identity with the amino acid sequence of SEQ ID NO: 680;
   c) amino acid sequences that have 3, 2, or 1 amino acid difference with the amino acid sequence of SEQ ID NO: 680.

In one embodiment, the ISVD comprises CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 402, 331, 332, 375, 212, 139, and 238.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 546, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 494, a CDR2 (AbM numbering) that is the amino acid sequence of SEQ ID NO: 547, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 495, a CDR2 (AbM numbering) that is the amino acid sequence of SEQ ID NO: 547, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 496, a CDR2 (AbM numbering) that is the amino acid sequence of SEQ ID NO: 547, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 493, a CDR2 (AbM numbering) that is the amino acid sequence of SEQ ID NO: 547, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 494, a CDR2 (AbM numbering) that is the amino acid sequence of SEQ ID NO: 541, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 495, a CDR2 (AbM numbering) that is the amino acid sequence of SEQ ID NO: 541, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 496, a CDR2 (AbM numbering) that is the amino acid sequence of SEQ ID NO: 541, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 493, a CDR2 (AbM numbering) that is the amino acid sequence of SEQ ID NO: 541, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 494, a CDR2 (AbM numbering) that is the amino acid sequence of SEQ ID NO: 549, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 495, a CDR2 (AbM numbering) that is the amino acid sequence of SEQ ID NO: 549, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 496, a CDR2 (AbM numbering) that is the amino acid sequence of SEQ ID NO: 549, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 493, a CDR2 (AbM numbering) that is the amino acid sequence of SEQ ID NO: 548, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 549, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 494, a CDR2 (AbM numbering) that is the amino acid sequence of SEQ ID NO: 547, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 540, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 542, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 544, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 530, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 495, a CDR2 (AbM numbering) that is the amino acid sequence of SEQ ID NO: 604, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 496, a CDR2 (AbM numbering) that is the amino acid sequence of SEQ ID NO: 604, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 604, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 494, a CDR2 (AbM numbering) that is the amino acid sequence of SEQ ID NO: 603, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 495, a CDR2 (AbM numbering) that is the amino acid sequence of SEQ ID NO: 603, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 496, a CDR2 (AbM numbering) that is the amino acid sequence of SEQ ID NO: 603, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 603, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 494, a CDR2 (AbM numbering) that is the amino acid sequence of SEQ ID NO: 543, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 496, a CDR2 (AbM numbering) that is the amino acid sequence of SEQ ID NO: 543, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 494, a CDR2 (AbM numbering) that is the amino acid sequence of SEQ ID NO: 604, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 495, a CDR2 (AbM numbering) that is the amino acid sequence of SEQ ID NO: 543, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 495, a CDR2 (AbM numbering) that is the amino acid sequence of SEQ ID NO: 547, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 535, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 496, a CDR2 (AbM numbering) that is the amino acid sequence of SEQ ID NO: 547, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 494, a CDR2 (AbM numbering) that is the amino acid sequence of SEQ ID NO: 541, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 496, a CDR2 (AbM numbering) that is the amino acid sequence of SEQ ID NO: 531, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 493, a CDR2 (AbM numbering) that is the amino acid sequence of SEQ ID NO: 531, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 494, a CDR2 (AbM numbering) that is the amino acid sequence of SEQ ID NO: 534, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 495, a CDR2 (AbM numbering) that is the amino acid sequence of SEQ ID NO: 534, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 496, a CDR2 (AbM numbering) that is the amino acid sequence of SEQ ID NO: 534, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 494, a CDR2 (AbM numbering) that is the amino acid sequence of SEQ ID NO: 531, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 495, a CDR2 (AbM numbering) that is the amino acid sequence of SEQ ID NO: 531, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 496, a CDR2 (AbM numbering) that is the amino acid sequence of SEQ ID NO: 531, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 531, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 495, a CDR2 (AbM numbering) that is the amino acid sequence of SEQ ID NO: 531, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 493, a CDR2 (AbM numbering) that is the amino acid sequence of SEQ ID NO: 529, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 602, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 527, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 528, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 601, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 532, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 534, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 494, a CDR2 (AbM numbering) that is the amino acid sequence of SEQ ID NO: 531, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 493, a CDR2 (AbM numbering) that is the amino acid sequence of SEQ ID NO: 534, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 496, a CDR2 (AbM numbering) that is the amino acid sequence of SEQ ID NO: 534, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 495, a CDR2 (AbM numbering) that is the amino acid sequence of SEQ ID NO: 541, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 496, a CDR2 (AbM numbering) that is the amino acid sequence of SEQ ID NO: 541, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 541, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 494, a CDR2 (AbM numbering) that is the amino acid sequence of SEQ ID NO: 549, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 496, a CDR2 (AbM numbering) that is the amino acid sequence of SEQ ID NO: 549, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 549, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 547, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 495, a CDR2 (AbM numbering) that is the amino acid sequence of SEQ ID NO: 549, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 494, a CDR2 (AbM numbering) that is the amino acid sequence of SEQ ID NO: 534, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 495, a CDR2 (AbM numbering) that is the amino acid sequence of SEQ ID NO: 534, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 494, a CDR2 (AbM numbering) that is the amino acid sequence of SEQ ID NO: 543, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 493, a CDR2 (AbM numbering) that is the amino acid sequence of SEQ ID NO: 578, and a CDR3 (AbM numbering) that is the amino acid sequence of SEQ ID NO: 707.

In one embodiment, the ISVD comprises a CDR1 (AbM numbering) that is the amino acid sequence of SEQ ID NO: 493, a CDR2 (AbM numbering) that is the amino acid sequence of SEQ ID NO: 578, and a CDR3 (AbM numbering) that is the amino acid sequence of SEQ ID NO: 703.

In one embodiment, the ISVD comprises a CDR1 (AbM numbering) that is the amino acid sequence of SEQ ID NO: 493, a CDR2 (AbM numbering) that is the amino acid sequence of SEQ ID NO: 578, and a CDR3 (AbM numbering) that is the amino acid sequence of SEQ ID NO: 672.

In one embodiment, the ISVD comprises a CDR1 (AbM numbering) that is the amino acid sequence of SEQ ID NO: 493, a CDR2 (AbM numbering) that is the amino acid sequence of SEQ ID NO: 578, and a CDR3 (AbM numbering) that is the amino acid sequence of SEQ ID NO: 665.

In one embodiment, the ISVD comprises a CDR1 (AbM numbering) that is the amino acid sequence of SEQ ID NO: 493, a CDR2 (AbM numbering) that is the amino acid sequence of SEQ ID NO: 592, and a CDR3 (AbM numbering) that is the amino acid sequence of SEQ ID NO: 701.

In one embodiment, the ISVD comprises a CDR1 (AbM numbering) that is the amino acid sequence of SEQ ID NO: 493, a CDR2 (AbM numbering) that is the amino acid sequence of SEQ ID NO: 592, and a CDR3 (AbM numbering) that is the amino acid sequence of SEQ ID NO: 703.

In one embodiment, the ISVD comprises a CDR1 (AbM numbering) that is the amino acid sequence of SEQ ID NO: 493, a CDR2 (AbM numbering) that is the amino acid sequence of SEQ ID NO: 592, and a CDR3 (AbM numbering) that is the amino acid sequence of SEQ ID NO: 672.

In one embodiment, the ISVD comprises a CDR1 (AbM numbering) that is the amino acid sequence of SEQ ID NO: 493, a CDR2 (AbM numbering) that is the amino acid sequence of SEQ ID NO: 592, and a CDR3 (AbM numbering) that is the amino acid sequence of SEQ ID NO: 665.

In one embodiment, the ISVD comprises a CDR1 (AbM numbering) that is the amino acid sequence of SEQ ID NO: 493, a CDR2 (AbM numbering) that is the amino acid sequence of SEQ ID NO: 592, and a CDR3 (AbM numbering) that is the amino acid sequence of SEQ ID NO: 707.

In one embodiment, the ISVD comprises a CDR1 (AbM numbering) that is the amino acid sequence of SEQ ID NO: 493, a CDR2 (AbM numbering) that is the amino acid sequence of SEQ ID NO: 593, and a CDR3 (AbM numbering) that is the amino acid sequence of SEQ ID NO: 701.

In one embodiment, the ISVD comprises a CDR1 (AbM numbering) that is the amino acid sequence of SEQ ID NO: 493, a CDR2 (AbM numbering) that is the amino acid sequence of SEQ ID NO: 593, and a CDR3 (AbM numbering) that is the amino acid sequence of SEQ ID NO: 703.

In one embodiment, the ISVD comprises a CDR1 (AbM numbering) that is the amino acid sequence of SEQ ID NO: 493, a CDR2 (AbM numbering) that is the amino acid sequence of SEQ ID NO: 593, and a CDR3 (AbM numbering) that is the amino acid sequence of SEQ ID NO: 672.

In one embodiment, the ISVD comprises a CDR1 (AbM numbering) that is the amino acid sequence of SEQ ID NO: 493, a CDR2 (AbM numbering) that is the amino acid sequence of SEQ ID NO: 593, and a CDR3 (AbM numbering) that is the amino acid sequence of SEQ ID NO: 665.

In one embodiment, the ISVD comprises a CDR1 (AbM numbering) that is the amino acid sequence of SEQ ID NO: 493, a CDR2 (AbM numbering) that is the amino acid sequence of SEQ ID NO: 593, and a CDR3 (AbM numbering) that is the amino acid sequence of SEQ ID NO: 707.

In one embodiment, the ISVD comprises a CDR1 (AbM numbering) that is the amino acid sequence of SEQ ID NO: 493, a CDR2 (AbM numbering) that is the amino acid sequence of SEQ ID NO: 594, and a CDR3 (AbM numbering) that is the amino acid sequence of SEQ ID NO: 701.

In one embodiment, the ISVD comprises a CDR1 (AbM numbering) that is the amino acid sequence of SEQ ID NO: 493, a CDR2 (AbM numbering) that is the amino acid sequence of SEQ ID NO: 594, and a CDR3 (AbM numbering) that is the amino acid sequence of SEQ ID NO: 703.

In one embodiment, the ISVD comprises a CDR1 (AbM numbering) that is the amino acid sequence of SEQ ID NO: 494, a CDR2 (AbM numbering) that is the amino acid sequence of SEQ ID NO: 537, and a CDR3 (AbM numbering) that is the amino acid sequence of SEQ ID NO: 670.

In one embodiment, the ISVD comprises a CDR1 (AbM numbering) that is the amino acid sequence of SEQ ID NO: 494, a CDR2 (AbM numbering) that is the amino acid sequence of SEQ ID NO: 593, and a CDR3 (AbM numbering) that is the amino acid sequence of SEQ ID NO: 670.

In one embodiment, the ISVD comprises a CDR1 (AbM numbering) that is the amino acid sequence of SEQ ID NO: 493, a CDR2 (AbM numbering) that is the amino acid sequence of SEQ ID NO: 559, and a CDR3 (AbM numbering) that is the amino acid sequence of SEQ ID NO: 645.

In one embodiment, the ISVD comprises a CDR1 (AbM numbering) that is the amino acid sequence of SEQ ID NO: 494, a CDR2 (AbM numbering) that is the amino acid sequence of SEQ ID NO: 553, and a CDR3 (AbM numbering) that is the amino acid sequence of SEQ ID NO: 670.

In one embodiment, the ISVD comprises a CDR1 (AbM numbering) that is the amino acid sequence of SEQ ID NO: 493, a CDR2 (AbM numbering) that is the amino acid sequence of SEQ ID NO: 598, and a CDR3 (AbM numbering) that is the amino acid sequence of SEQ ID NO: 645.

In one embodiment, the ISVD comprises a CDR1 (AbM numbering) that is the amino acid sequence of SEQ ID NO: 493, a CDR2 (AbM numbering) that is the amino acid sequence of SEQ ID NO: 593, and a CDR3 (AbM numbering) that is the amino acid sequence of SEQ ID NO: 644.

In one embodiment, the ISVD comprises a CDR1 (AbM numbering) that is the amino acid sequence of SEQ ID NO: 493, a CDR2 (AbM numbering) that is the amino acid sequence of SEQ ID NO: 557, and a CDR3 (AbM numbering) that is the amino acid sequence of SEQ ID NO: 645.

In one embodiment, the ISVD comprises a CDR1 (AbM numbering) that is the amino acid sequence of SEQ ID NO: 494, a CDR2 (AbM numbering) that is the amino acid sequence of SEQ ID NO: 550, and a CDR3 (AbM numbering) that is the amino acid sequence of SEQ ID NO: 670.

In one embodiment, the ISVD comprises a CDR1 (AbM numbering) that is the amino acid sequence of SEQ ID NO: 494, a CDR2 (AbM numbering) that is the amino acid sequence of SEQ ID NO: 595, and a CDR3 (AbM numbering) that is the amino acid sequence of SEQ ID NO: 670.

In one embodiment, the ISVD comprises a CDR1 (AbM numbering) that is the amino acid sequence of SEQ ID NO: 494, a CDR2 (AbM numbering) that is the amino acid sequence of SEQ ID NO: 555, and a CDR3 (AbM numbering) that is the amino acid sequence of SEQ ID NO: 670.

In one embodiment, the ISVD comprises a CDR1 (AbM numbering) that is the amino acid sequence of SEQ ID NO: 494, a CDR2 (AbM numbering) that is the amino acid sequence of SEQ ID NO: 559, and a CDR3 (AbM numbering) that is the amino acid sequence of SEQ ID NO: 670.

In one embodiment, the ISVD comprises a CDR1 (AbM numbering) that is the amino acid sequence of SEQ ID NO: 493, a CDR2 (AbM numbering) that is the amino acid sequence of SEQ ID NO: 550, and a CDR3 (AbM numbering) that is the amino acid sequence of SEQ ID NO: 645.

In one embodiment, the ISVD comprises a CDR1 (AbM numbering) that is the amino acid sequence of SEQ ID NO: 493, a CDR2 (AbM numbering) that is the amino acid sequence of SEQ ID NO: 553, and a CDR3 (AbM numbering) that is the amino acid sequence of SEQ ID NO: 645.

In one embodiment, the ISVD comprises a CDR1 (AbM numbering) that is the amino acid sequence of SEQ ID NO: 494, a CDR2 (AbM numbering) that is the amino acid sequence of SEQ ID NO: 591, and a CDR3 (AbM numbering) that is the amino acid sequence of SEQ ID NO: 670.

In one embodiment, the ISVD comprises a CDR1 (AbM numbering) that is the amino acid sequence of SEQ ID NO: 493, a CDR2 (AbM numbering) that is the amino acid sequence of SEQ ID NO: 578, and a CDR3 (AbM numbering) that is the amino acid sequence of SEQ ID NO: 701.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 701.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 703.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 672.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 665.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 707.

In one embodiment, the ISVD comprises a CDR1 (AbM numbering) that is the amino acid sequence of SEQ ID NO: 493, a CDR2 (AbM numbering) that is the amino acid sequence of SEQ ID NO: 594, and a CDR3 (AbM numbering) that is the amino acid sequence of SEQ ID NO: 672.

In one embodiment, the ISVD comprises a CDR1 (AbM numbering) that is the amino acid sequence of SEQ ID NO: 493, a CDR2 (AbM numbering) that is the amino acid sequence of SEQ ID NO: 603, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 496, a CDR2 (AbM numbering) that is the amino acid sequence of SEQ ID NO: 543, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 493, a CDR2 (AbM numbering) that is the amino acid sequence of SEQ ID NO: 539, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 678.

In one embodiment, the ISVD comprises a CDR1 (AbM numbering) that is the amino acid sequence of SEQ ID NO: 494, a CDR2 (AbM numbering) that is the amino acid sequence of SEQ ID NO: 604, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 495, a CDR2 (AbM numbering) that is the amino acid sequence of SEQ ID NO: 604, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 496, a CDR2 (AbM numbering) that is the amino acid sequence of SEQ ID NO: 604, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 493, a CDR2 (AbM numbering) that is the amino acid sequence of SEQ ID NO: 604, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 494, a CDR2 (AbM numbering) that is the amino acid sequence of SEQ ID NO: 603, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 495, a CDR2 (AbM numbering) that is the amino acid sequence of SEQ ID NO: 603, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 495, a CDR2 (AbM numbering) that is the amino acid sequence of SEQ ID NO: 543, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 496, a CDR2 (AbM numbering) that is the amino acid sequence of SEQ ID NO: 603, and a CDR3 (AbM numbering) that is the amino acid sequence of SEQ ID NO: 674.

In one embodiment, the ISVD comprises a CDR1 (AbM numbering) that is the amino acid sequence of SEQ ID NO: 493, a CDR2 (AbM numbering) that is the amino acid sequence of SEQ ID NO: 594, and a CDR3 (AbM numbering) that is the amino acid sequence of SEQ ID NO: 707.

In one embodiment, the ISVD comprises a CDR1 (AbM numbering) that is the amino acid sequence of SEQ ID NO: 493, a CDR2 (AbM numbering) that is the amino acid sequence of SEQ ID NO: 594, and a CDR3 (AbM numbering) that is the amino acid sequence of SEQ ID NO: 665.

In one embodiment, the ISVD comprises a CDR1 (AbM numbering) that is the amino acid sequence of SEQ ID NO: 493, a CDR2 (AbM numbering) that is the amino acid sequence of SEQ ID NO: 595, and a CDR3 (AbM numbering) that is the amino acid sequence of SEQ ID NO: 701.

In one embodiment, the ISVD comprises a CDR1 (AbM numbering) that is the amino acid sequence of SEQ ID NO: 493, a CDR2 (AbM numbering) that is the amino acid sequence of SEQ ID NO: 595, and a CDR3 (AbM numbering) that is the amino acid sequence of SEQ ID NO: 703.

In one embodiment, the ISVD comprises a CDR1 (AbM numbering) that is the amino acid sequence of SEQ ID NO: 493, a CDR2 (AbM numbering) that is the amino acid sequence of SEQ ID NO: 595, and a CDR3 (AbM numbering) that is the amino acid sequence of SEQ ID NO: 672.

In one embodiment, the ISVD comprises a CDR1 (AbM numbering) that is the amino acid sequence of SEQ ID NO: 493, a CDR2 (AbM numbering) that is the amino acid sequence of SEQ ID NO: 595, and a CDR3 (AbM numbering) that is the amino acid sequence of SEQ ID NO: 665.

In one embodiment, the ISVD comprises a CDR1 (AbM numbering) that is the amino acid sequence of SEQ ID NO: 493, a CDR2 (AbM numbering) that is the amino acid sequence of SEQ ID NO: 595, and a CDR3 (AbM numbering) that is the amino acid sequence of SEQ ID NO: 707.

In one embodiment, the ISVD comprises a CDR1 (AbM numbering) that is the amino acid sequence of SEQ ID NO: 494, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 494, a CDR2 (AbM numbering) that is the amino acid sequence of SEQ ID NO: 542, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 676.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 677.

In one embodiment, the ISVD comprises a CDR1 (AbM numbering) that is the amino acid sequence of SEQ ID NO: 493, a CDR2 (AbM numbering) that is the amino acid sequence of SEQ ID NO: 575, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 570, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 566, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 564, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 567, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 572, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 580, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 587, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 588, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 583, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 568, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 584, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 560, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 586, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 2393, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 585, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 561, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 582, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 581, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 592, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 552, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 558, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 590, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 597, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 579, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 577, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 571, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 567, and a CDR3 (AbM numbering) that is the amino acid sequence of SEQ ID NO: 690.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 702.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 667.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 673.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 663.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 666.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 661.

In one embodiment, the ISVD comprises a CDR1 (AbM numbering) that is the amino acid sequence of SEQ ID NO: 493, a CDR2 (AbM numbering) that is the amino acid sequence of SEQ ID NO: 576, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 573, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 562, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 574, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 563, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 556, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 589, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 599, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 681.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 662.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 538, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 594, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 554, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 497, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 498, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 499, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 500, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 501, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 502, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 503, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 504, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 505, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 506, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 507, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 508, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 509, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 510, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 511, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 536, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 700.

In one embodiment, the ISVD comprises a CDR1 (AbM numbering) that is the amino acid sequence of SEQ ID NO: 493, a CDR2 (AbM numbering) that is the amino acid sequence of SEQ ID NO: 569, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 565, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 675.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 687.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 706.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 689.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 669.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 664.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 704.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 684.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 665.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 707.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 704.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 670.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 664.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 675.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 649.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 650.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 660.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 648.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 710.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 711.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 657.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 705.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 653.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 647.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 651.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 679.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 705.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 706.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 680.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 665.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 707.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 689.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 687.

In one embodiment, the ISVD comprises a CDR1 (AbM numbering) that is the amino acid sequence of SEQ ID NO: 493, a CDR2 (AbM numbering) that is the amino acid sequence of SEQ ID NO: 551, and a CDR3 (AbM numbering) that is the amino acid sequence of SEQ ID NO: 684.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 655.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 699.

In one embodiment, the ISVD comprises a CDR1 (AbM numbering) that is the amino acid sequence of SEQ ID NO: 493, a CDR2 (AbM numbering) that is the amino acid sequence of SEQ ID NO: 543, and a CDR3 (AbM numbering) that is the amino acid sequence of SEQ ID NO: 671.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 683.

In one embodiment, the ISVD comprises a CDR1 (AbM numbering) that is the amino acid sequence of SEQ ID NO: 2392, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 512, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 513, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 692.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 694.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 689.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 688.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 695.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 682.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 687.

In one embodiment, the ISVD comprises a CDR1 (AbM numbering) that is the amino acid sequence of SEQ ID NO: 494, a CDR2 (AbM numbering) that is the amino acid sequence of SEQ ID NO: 600, and a CDR3 (AbM numbering) that is the amino acid sequence of SEQ ID NO: 670.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 696.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 685.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 686.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 697.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 698.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 684.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 708.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 709.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 668.

In one embodiment, the ISVD comprises a CDR1 (AbM numbering) that is the amino acid sequence of SEQ ID NO: 494, a CDR2 (AbM numbering) that is the amino acid sequence of SEQ ID NO: 557, and a CDR3 (AbM numbering) that is the amino acid sequence of SEQ ID NO: 670.

In one embodiment, the ISVD comprises a CDR1 (AbM numbering) that is the amino acid sequence of SEQ ID NO: 514, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 693.

In one embodiment, the ISVD comprises a CDR1 (AbM numbering) that is the amino acid sequence of SEQ ID NO: 496, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 646.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 652.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 659.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 658.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 656.

In one embodiment, the ISVD comprises a CDR1 (AbM numbering) that is the amino acid sequence of SEQ ID NO: 493, a CDR2 (AbM numbering) that is the amino acid sequence of SEQ ID NO: 526, and a CDR3 (AbM numbering) that is the amino acid sequence of SEQ ID NO: 654.

In one embodiment, the ISVD comprises a CDR1 (AbM numbering) that is the amino acid sequence of SEQ ID NO: 515, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 516, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 517, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 518, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 519, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 520, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 495, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 521, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 522, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 523, a CDR2 (AbM numbering) that is the amino acid sequence of SEQ ID NO: 526, and a CDR3 (AbM numbering) that is the amino acid sequence of SEQ ID NO: 691.

In one embodiment, the ISVD comprises a CDR1 (AbM numbering) that is the amino acid sequence of SEQ ID NO: 494, a CDR2 (AbM numbering) that is the amino acid sequence of SEQ ID NO: 596, and a CDR3 (AbM numbering) that is the amino acid sequence of SEQ ID NO: 670.

In some embodiments, the ISVD additionally comprises V2, Q3, L4, R45, W103 and/or Q105 (Kabat numbering). In some embodiments, the ISVD additionally comprises R45 (Kabat numbering). In some embodiments, the ISVD additionally comprises W103 (Kabat numbering). In some embodiments, the ISVD additionally comprises Q105 (Kabat numbering). In some embodiments, the ISVD additionally comprises R45 and Q105 (Kabat numbering). In some embodiments, the ISVD additionally comprises R45 and W103 (Kabat numbering). In some embodiments, the ISVD additionally comprises R45, W103 and Q105 (Kabat numbering). In some embodiments, the ISVD additionally comprises V2, Q3, L4, R45, W103 and Q105 (Kabat numbering).

In some embodiments, the ISVD comprises an amino acid sequence having 80% amino acid sequence identity with one of the amino acid sequences of SEQ ID NOs: 1-488, in which for the purposes of determining the degree of amino acid identity, the amino acid residues that form the CDR sequences are disregarded; and in which: preferably one or more of the amino acid residues at positions 11, 37, 44, 45, 47, 83, 84, 103, 104 and 108 according to the Kabat numbering are chosen from the Hallmark residues mentioned in Table 1.

In some embodiments, the amino acid residue of the ISVD at the following position: 45 (Kabat numbering) is capable of forming salt bridges with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C., preferably wherein the ISVD comprises at least the following amino acid residue: R45 (Kabat numbering).

In some embodiments, R45 (Kabat numbering) of the ISVD is capable of forming salt bridges with the amino acid residue E136 of human PD-1 protein, when numbered in accordance with SEQ ID NO: 4703,

In some embodiments, the ISVD essentially consists of a heavy chain variable domain sequence that is derived from a conventional four-chain antibody or that essentially consists of a heavy chain variable domain sequence that is derived from a heavy chain antibody.

In some embodiments, the ISVD essentially consists of a VHH, a humanized VHH, a camelized VH, a domain antibody, a single domain antibody, or a dAb.

In some embodiments, the ISVD is a humanized ISVD that is chosen from SEQ ID NO: 1-401, 403-488 or from the group consisting of amino acid sequences that have more than 80%, preferably more than 90%, more preferably more than 95%, such as 99% or more amino acid sequence identity with at least one of the amino acid sequences of SEQ ID NOs: 1-488.

In some embodiments, the ISVD is a humanized ISVD that is chosen from SEQ ID NO: 331, 332, 375, 212, 139, and 238 or from the group consisting of amino acid sequences that have more than 80%, preferably more than 90%, more preferably more than 95%, such as 99% or more amino acid sequence identity with at least one of the amino acid sequences of SEQ ID NOs: 402, 331, 332, 375, 212, 139, 238.

In some embodiments, the ISVD is chosen from the group consisting of SEQ ID NO: 402 and SEQ ID NOs: 1-401, 403-488.

In some embodiments, the ISVD specifically binds to human PD-1 with a dissociation constant (K_{D}) of 10⁻⁹ moles/litre or less, 10⁻¹⁰ moles/litre or less, such as 5×10⁻⁹ to 10⁻¹² moles/litre or less, and preferably 10⁻¹⁰ to 10⁻¹² moles/litre or less and more preferably 5×10⁻¹⁰ to 10⁻¹¹ moles/litre, 5×10⁻¹⁰ to 5×10⁻¹¹ moles/litre, 10⁻¹⁰ to 10⁻¹² moles/litre, or 10⁻¹⁰ to 10⁻¹¹ moles/litre, such as 4.3×10⁻¹¹ moles/litre, as determined by Surface Plasmon Resonance, e.g., measured at 25° C.

In some embodiments, the ISVD specifically binds to human PD-1 with a kₒₙ-rate of between 10⁵ M⁻¹ s⁻¹ to about 10⁷ M⁻¹ s⁻¹, preferably between 5×10⁵ M⁻¹ s⁻¹ and 10⁷ M⁻¹ s⁻¹, more preferably between 10⁵ M⁻¹ s⁻¹ and 5×10⁶ M⁻¹ s⁻¹, such as between 10⁵ M⁻¹ s⁻¹ and 1×10⁶ M⁻¹ s⁻¹, such as 3×10⁵ M⁻¹ s⁻¹, as determined by Surface Plasmon Resonance, e.g., measured at 25° C.

In some embodiments, the ISVD specifically binds to human PD-1 with a k_{off} rate between 10⁻³ s⁻¹ (t1/2=0.69 s) and 10⁻⁸ s⁻¹ (providing a near irreversible complex with a t1/2 of multiple days), preferably 10⁻³ s⁻¹ and 5×10⁻⁶ s⁻¹, more preferably between 5×10⁻⁴ s⁻¹ and 5×10⁶ s⁻¹, such as between 5×10⁻⁴ s⁻¹ and 10⁻⁵ s⁻¹, such as 1.3×10⁻⁵ s⁻¹, determined by Surface Plasmon Resonance, e.g., measured at 25° C.

In some embodiments, the ISVD specifically binds to human and cyno PD-1 and does not bind to CD28, CTLA-4, ICOS, and BTLA or other members of the CD28 receptor family.

In some embodiments, the ISVD antagonizes an activity of PD-L1.

In some embodiments, the ISVD blocks the interaction of human PD-1 with human PD-L1 with a potency (IC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 10⁻⁹ M, between 10⁻¹⁰ M and 10⁻⁸ M or between 10⁻⁹ M and 10⁻⁸ M, such as 5.11×10⁻⁹ M, for example, as measured in a FACS competition assay on CHO cells expressing human PD-1 with hPD-L1 as a competitor.

In some embodiments, the ISVD blocks the interaction of cyno PD-1 with cyno PD-L1 with a potency (IC50 value) of 10⁻⁷ M or lower, of 5×10⁻⁸ M or lower, more preferably of 10⁻⁸ M or lower, or even of 5×10⁻⁹ M or lower, such as between 10⁻¹¹ M and 10⁻⁷ M, between 10⁻¹¹ M and 10⁻⁸ M, between 10⁻¹⁰ M and 10⁻⁷ M, between 10⁻¹⁰ M and 10⁻⁹ M, between 10⁻¹⁰ M and 10⁻⁸ M, between 10⁻⁹ M and 10⁻⁸ M or between 10⁻⁹ M and 10⁻⁷ M, such as 5.76×10⁻⁸ M, for example, as measured in a FACS competition assay on CHO cells expressing cyno PD-1 with hPD-L1 as a competitor.

In some embodiments, the ISVD blocks the binding of hPD-1 to hPD-L1 by at least 50%, such as at least 60%, 70%, 80%, 90%, 95%, 99% or even more, as determined by ligand competition, AlphaScreen, or competitive binding assays (such as competition ELISA or competition FACS).

In some embodiments, the ISVD induces T-cell activation with a potency (EC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 10⁻⁸ M, between 10⁻¹⁰ M and 10⁻⁹ M or between 10⁻⁹ M and 10⁻⁸ M, such as 2.2×10⁻⁹ M or 2.8×10⁻⁹ M, as determined in a TT assay.

Specific examples of such ISVDs that specifically bind to PD-1 have one or more, or all, framework regions as indicated for A0294047E02 and sequence optimized variants thereof in Table A-2 and A-6 (in addition to the CDRs as defined above). In one embodiment, the ISVD comprises or consists of the full amino acid sequence of A0294047E02 (SEQ ID NO: 402), A029400054 (SEQ ID NO: 1), A029404968 (SEQ ID NO: 2), A029404967 (SEQ ID NO: 3), A029404966 (SEQ ID NO: 4), A029404965 (SEQ ID NO: 5), A029404964 (SEQ ID NO: 6), A029404963 (SEQ ID NO: 7), A029404962 (SEQ ID NO: 8), A029404961 (SEQ ID NO: 9), A029404960 (SEQ ID NO: 10), A029404959 (SEQ ID NO: 11), A029404958 (SEQ ID NO: 12), A029404957 (SEQ ID NO: 13), A029404969 (SEQ ID NO: 14), A029404956 (SEQ ID NO: 15), A029404954 (SEQ ID NO: 16), A029404953 (SEQ ID NO: 17), A029404952 (SEQ ID NO: 18), A029404951 (SEQ ID NO: 19), A029404950 (SEQ ID NO: 20), A029404949 (SEQ ID NO: 21), A029404948 (SEQ ID NO: 22), A029404947 (SEQ ID NO: 23), A029404946 (SEQ ID NO: 24), A029404945 (SEQ ID NO: 25), A029404944 (SEQ ID NO: 26), A029404943 (SEQ ID NO: 27), A029404955 (SEQ ID NO: 28), A029404970 (SEQ ID NO: 29), A029404971 (SEQ ID NO: 30), A029404972 (SEQ ID NO: 31), A029404877 (SEQ ID NO: 32), A029404998 (SEQ ID NO: 33), A029404997 (SEQ ID NO: 34), A029404996 (SEQ ID NO: 35), A029404995 (SEQ ID NO: 36), A029404994 (SEQ ID NO: 37), A029404993 (SEQ ID NO: 38), A029404992 (SEQ ID NO: 39), A029404991 (SEQ ID NO: 40), A029404990 (SEQ ID NO: 41), A029404989 (SEQ ID NO: 42), A029404988 (SEQ ID NO: 43), A029404987 (SEQ ID NO: 44), A029404986 (SEQ ID NO: 45), A029404985 (SEQ ID NO: 46), A029404984 (SEQ ID NO: 47), A029404983 (SEQ ID NO: 48), A029404982 (SEQ ID NO: 49), A029404981 (SEQ ID NO: 50), A029404980 (SEQ ID NO: 51), A029404979 (SEQ ID NO: 52), A029404978 (SEQ ID NO: 53), A029404977 (SEQ ID NO: 54), A029404976 (SEQ ID NO: 55), A029404975 (SEQ ID NO: 56), A029404974 (SEQ ID NO: 57), A029404973 (SEQ ID NO: 58), A029404942 (SEQ ID NO: 59), A029404878 (SEQ ID NO: 60), A029404941 (SEQ ID NO: 61), A029404939 (SEQ ID NO: 62), A029404907 (SEQ ID NO: 63), A029404906 (SEQ ID NO: 64), A029404905 (SEQ ID NO: 65), A029404904 (SEQ ID NO: 66), A029404903 (SEQ ID NO: 67), A029404901 (SEQ ID NO: 68), A029404900 (SEQ ID NO: 69), A029404899 (SEQ ID NO: 70), A029404898 (SEQ ID NO: 71), A029404897 (SEQ ID NO: 72), A029404896 (SEQ ID NO: 73), A029404895 (SEQ ID NO: 74), A029404908 (SEQ ID NO: 75), A029404893 (SEQ ID NO: 76), A029404891 (SEQ ID NO: 77), A029404890 (SEQ ID NO: 78), A029404889 (SEQ ID NO: 79), A029404888 (SEQ ID NO: 80), A029404887 (SEQ ID NO: 81), A029404886 (SEQ ID NO: 82), A029404885 (SEQ ID NO: 83), A029404884 (SEQ ID NO: 84), A029404883 (SEQ ID NO: 85), A029404882 (SEQ ID NO: 86), A029404881 (SEQ ID NO: 87), A029404880 (SEQ ID NO: 88), A029404892 (SEQ ID NO: 89), A029404909 (SEQ ID NO: 90), A029404910 (SEQ ID NO: 91), A029404911 (SEQ ID NO: 92), A029404938 (SEQ ID NO: 93), A029404937 (SEQ ID NO: 94), A029404936 (SEQ ID NO: 95), A029404935 (SEQ ID NO: 96), A029404934 (SEQ ID NO: 97), A029404933 (SEQ ID NO: 98), A029404932 (SEQ ID NO: 99), A029404931 (SEQ ID NO: 100), A029404930 (SEQ ID NO: 101), A029404929 (SEQ ID NO: 102), A029404928 (SEQ ID NO: 103), A029404927 (SEQ ID NO: 104), A029404926 (SEQ ID NO: 105), A029404925 (SEQ ID NO: 106), A029404924 (SEQ ID NO: 107), A029404923 (SEQ ID NO: 108), A029404922 (SEQ ID NO: 109), A029404921 (SEQ ID NO: 110), A029404920 (SEQ ID NO: 111), A029404919 (SEQ ID NO: 112), A029404918 (SEQ ID NO: 113), A029404917 (SEQ ID NO: 114), A029404916 (SEQ ID NO: 115), A029404915 (SEQ ID NO: 116), A029404914 (SEQ ID NO: 117), A029404913 (SEQ ID NO: 118), A029404912 (SEQ ID NO: 119), A029404940 (SEQ ID NO: 120), A029404879 (SEQ ID NO: 121), A029404999 (SEQ ID NO: 122), A029405013 (SEQ ID NO: 123), A029405135 (SEQ ID NO: 124), A029405134 (SEQ ID NO: 125), A029405133 (SEQ ID NO: 126), A029405132 (SEQ ID NO: 127), A029405131 (SEQ ID NO: 128), A029405130 (SEQ ID NO: 129), A029405129 (SEQ ID NO: 130), A029405128 (SEQ ID NO: 131), A029405127 (SEQ ID NO: 132), A029405126 (SEQ ID NO: 133), A029405125 (SEQ ID NO: 134), A029405124 (SEQ ID NO: 135), A029403867 (SEQ ID NO: 136), A029405137 (SEQ ID NO: 137), A029405121 (SEQ ID NO: 138), A029405120 (SEQ ID NO: 139), A029405119 (SEQ ID NO: 140), A029405118 (SEQ ID NO: 141), A029405117 (SEQ ID NO: 142), A029405116 (SEQ ID NO: 143), A029405115 (SEQ ID NO: 144), A029405114 (SEQ ID NO: 145), A029405113 (SEQ ID NO: 146), A029405112 (SEQ ID NO: 147), A029405111 (SEQ ID NO: 148), A029405110 (SEQ ID NO: 149), A029405136 (SEQ ID NO: 150), A029403869 (SEQ ID NO: 151), A029405138 (SEQ ID NO: 152), A029405188 (SEQ ID NO: 153), A029405187 (SEQ ID NO: 154), A029405186 (SEQ ID NO: 155), A029405176 (SEQ ID NO: 156), A029405165 (SEQ ID NO: 157), A029405163 (SEQ ID NO: 158), A029405162 (SEQ ID NO: 159), A029405161 (SEQ ID NO: 160), A029405160 (SEQ ID NO: 161), A029405159 (SEQ ID NO: 162), A029405158 (SEQ ID NO: 163), A029405157 (SEQ ID NO: 164), A029405156 (SEQ ID NO: 165), A029405155 (SEQ ID NO: 166), A029405154 (SEQ ID NO: 167), A029405153 (SEQ ID NO: 168), A029405149 (SEQ ID NO: 169), A029405148 (SEQ ID NO: 170), A029405147 (SEQ ID NO: 171), A029405146 (SEQ ID NO: 172), A029405145 (SEQ ID NO: 173), A029405144 (SEQ ID NO: 174), A029405143 (SEQ ID NO: 175), A029405142 (SEQ ID NO: 176), A029405141 (SEQ ID NO: 177), A029405140 (SEQ ID NO: 178), A029405109 (SEQ ID NO: 179), A029405122 (SEQ ID NO: 180), A029405123 (SEQ ID NO: 181), A029405014 (SEQ ID NO: 182), A029405001 (SEQ ID NO: 183), A029405106 (SEQ ID NO: 184), A029405030 (SEQ ID NO: 185), A029405028 (SEQ ID NO: 186), A029405027 (SEQ ID NO: 187), A029405026 (SEQ ID NO: 188), A029405025 (SEQ ID NO: 189), A029405024 (SEQ ID NO: 190), A029405021 (SEQ ID NO: 191), A029405020 (SEQ ID NO: 192), A029405019 (SEQ ID NO: 193), A029405018 (SEQ ID NO: 194), A029405017 (SEQ ID NO: 195), A029405016 (SEQ ID NO: 196), A029405105 (SEQ ID NO: 197), A029405032 (SEQ ID NO: 198), A029405033 (SEQ ID NO: 199), A029405012 (SEQ ID NO: 200), A029405011 (SEQ ID NO: 201), A029405010 (SEQ ID NO: 202), A029405009 (SEQ ID NO: 203), A029405008 (SEQ ID NO: 204), A029405007 (SEQ ID NO: 205), A029405006 (SEQ ID NO: 206), A029405005 (SEQ ID NO: 207), A029405004 (SEQ ID NO: 208), A029405003 (SEQ ID NO: 209), A029405002 (SEQ ID NO: 210), A029405031 (SEQ ID NO: 211), A029405107 (SEQ ID NO: 212), A029405029 (SEQ ID NO: 213), A029405108 (SEQ ID NO: 214), A029405015 (SEQ ID NO: 215), A029405104 (SEQ ID NO: 216), A029405103 (SEQ ID NO: 217), A029405102 (SEQ ID NO: 218), A029405101 (SEQ ID NO: 219), A029405100 (SEQ ID NO: 220), A029405099 (SEQ ID NO: 221), A029405098 (SEQ ID NO: 222), A029405097 (SEQ ID NO: 223), A029405096 (SEQ ID NO: 224), A029405095 (SEQ ID NO: 225), A029405094 (SEQ ID NO: 226), A029405093 (SEQ ID NO: 227), A029405092 (SEQ ID NO: 228), A029405091 (SEQ ID NO: 229), A029405063 (SEQ ID NO: 230), A029405047 (SEQ ID NO: 231), A029405046 (SEQ ID NO: 232), A029405045 (SEQ ID NO: 233), A029405044 (SEQ ID NO: 234), A029405043 (SEQ ID NO: 235), A029405042 (SEQ ID NO: 236), A029405041 (SEQ ID NO: 237), A029405040 (SEQ ID NO: 238), A029405039 (SEQ ID NO: 239), A029405035 (SEQ ID NO: 240), A029405034 (SEQ ID NO: 241), A029405000 (SEQ ID NO: 242), A029404876 (SEQ ID NO: 243), A029404875 (SEQ ID NO: 244), A029404874 (SEQ ID NO: 245), A029401559 (SEQ ID NO: 246), A029401558 (SEQ ID NO: 247), A029401557 (SEQ ID NO: 248), A029401556 (SEQ ID NO: 249), A029401555 (SEQ ID NO: 250), A029401554 (SEQ ID NO: 251), A029401540 (SEQ ID NO: 252), A029401538 (SEQ ID NO: 253), A029401536 (SEQ ID NO: 254), A029401535 (SEQ ID NO: 255), A029401562 (SEQ ID NO: 256), A029401534 (SEQ ID NO: 257), A029401531 (SEQ ID NO: 258), A029401529 (SEQ ID NO: 259), A029401528 (SEQ ID NO: 260), A029401527 (SEQ ID NO: 261), A029401525 (SEQ ID NO: 262), A029401524 (SEQ ID NO: 263), A029401523 (SEQ ID NO: 264), A029401514 (SEQ ID NO: 265), A029401511 (SEQ ID NO: 266), A029401504 (SEQ ID NO: 267), A029401501 (SEQ ID NO: 268), A029401500 (SEQ ID NO: 269), A029401533 (SEQ ID NO: 270), A029401563 (SEQ ID NO: 271), A029401564 (SEQ ID NO: 272), A029401570 (SEQ ID NO: 273), A029401640 (SEQ ID NO: 274), A029401639 (SEQ ID NO: 275), A029401638 (SEQ ID NO: 276), A029401635 (SEQ ID NO: 277), A029401634 (SEQ ID NO: 278), A029401633 (SEQ ID NO: 279), A029401632 (SEQ ID NO: 280), A029401631 (SEQ ID NO: 281), A029401622 (SEQ ID NO: 282), A029401616 (SEQ ID NO: 283), A029401610 (SEQ ID NO: 284), A029401609 (SEQ ID NO: 285), A029401608 (SEQ ID NO: 286), A029401604 (SEQ ID NO: 287), A029401603 (SEQ ID NO: 288), A029401601 (SEQ ID NO: 289), A029401600 (SEQ ID NO: 290), A029401599 (SEQ ID NO: 291), A029401597 (SEQ ID NO: 292), A029401596 (SEQ ID NO: 293), A029401595 (SEQ ID NO: 294), A029401592 (SEQ ID NO: 295), A029401580 (SEQ ID NO: 296), A029401579 (SEQ ID NO: 297), A029401575 (SEQ ID NO: 298), A029401573 (SEQ ID NO: 299), A029401571 (SEQ ID NO: 300), A029401498 (SEQ ID NO: 301), A029401641 (SEQ ID NO: 302), A029401496 (SEQ ID NO: 303), A029401493 (SEQ ID NO: 304), A029400099 (SEQ ID NO: 305), A029400098 (SEQ ID NO: 306), A029400096 (SEQ ID NO: 307), A029400095 (SEQ ID NO: 308), A029400094 (SEQ ID NO: 309), A029400093 (SEQ ID NO: 310), A029400092 (SEQ ID NO: 311), A029400091 (SEQ ID NO: 312), A029400090 (SEQ ID NO: 313), A029400089 (SEQ ID NO: 314), A029400088 (SEQ ID NO: 315), A029400087 (SEQ ID NO: 316), A029400100 (SEQ ID NO: 317), A029400086 (SEQ ID NO: 318), A029400084 (SEQ ID NO: 319), A029400083 (SEQ ID NO: 320), A029400082 (SEQ ID NO: 321), A029400081 (SEQ ID NO: 322), A029400080 (SEQ ID NO: 323), A029400079 (SEQ ID NO: 324), A029400078 (SEQ ID NO: 325), A029400077 (SEQ ID NO: 326), A029400076 (SEQ ID NO: 327), A029400057 (SEQ ID NO: 328), A029400056 (SEQ ID NO: 329), A029400055 (SEQ ID NO: 330), A029400085 (SEQ ID NO: 331), A029400101 (SEQ ID NO: 332), A029400102 (SEQ ID NO: 333), A029400103 (SEQ ID NO: 334), A029401492 (SEQ ID NO: 335), A029401491 (SEQ ID NO: 336), A029401490 (SEQ ID NO: 337), A029401489 (SEQ ID NO: 338), A029401488 (SEQ ID NO: 339), A029401480 (SEQ ID NO: 340), A029401479 (SEQ ID NO: 341), A029401474 (SEQ ID NO: 342), A029401467 (SEQ ID NO: 343), A029401465 (SEQ ID NO: 344), A029401464 (SEQ ID NO: 345), A029401463 (SEQ ID NO: 346), A029401462 (SEQ ID NO: 347), A029401461 (SEQ ID NO: 348), A029401459 (SEQ ID NO: 349), A029401458 (SEQ ID NO: 350), A029401457 (SEQ ID NO: 351), A029401456 (SEQ ID NO: 352), A029401455 (SEQ ID NO: 353), A029401454 (SEQ ID NO: 354), A029400199 (SEQ ID NO: 355), A029400168 (SEQ ID NO: 356), A029400167 (SEQ ID NO: 357), A029400166 (SEQ ID NO: 358), A029400165 (SEQ ID NO: 359), A029400164 (SEQ ID NO: 360), A029400162 (SEQ ID NO: 361), A029401495 (SEQ ID NO: 362), A029401642 (SEQ ID NO: 363), A029401561 (SEQ ID NO: 364), A029401560 (SEQ ID NO: 365), A029401643 (SEQ ID NO: 366), A029404031 (SEQ ID NO: 367), A029404720 (SEQ ID NO: 368), A029404718 (SEQ ID NO: 369), A029404717 (SEQ ID NO: 370), A029404716 (SEQ ID NO: 371), A029404715 (SEQ ID NO: 372), A029404714 (SEQ ID NO: 373), A029404713 (SEQ ID NO: 374), A029404712 (SEQ ID NO: 375), A029404711 (SEQ ID NO: 376), A029404710 (SEQ ID NO: 377), A029404035 (SEQ ID NO: 378), A029404033 (SEQ ID NO: 379), A029404873 (SEQ ID NO: 380), A029404722 (SEQ ID NO: 381), A029404723 (SEQ ID NO: 382), A029404014 (SEQ ID NO: 383), A029404013 (SEQ ID NO: 384), A029404012 (SEQ ID NO: 385), A029404008 (SEQ ID NO: 386), A029403984 (SEQ ID NO: 387), A029403981 (SEQ ID NO: 388), A029403973 (SEQ ID NO: 389), A029403967 (SEQ ID NO: 390), A029403964 (SEQ ID NO: 391), A029403962 (SEQ ID NO: 392), A029403957 (SEQ ID NO: 393), A029404721 (SEQ ID NO: 394), A029403949 (SEQ ID NO: 395), A029403945 (SEQ ID NO: 396), A029404029 (SEQ ID NO: 397), A029403943 (SEQ ID NO: 398), A029404872 (SEQ ID NO: 399), A029404871 (SEQ ID NO: 400), A029404870 (SEQ ID NO: 401), A029404745 (SEQ ID NO: 403), A029404744 (SEQ ID NO: 404), A029404743 (SEQ ID NO: 405), A029404742 (SEQ ID NO: 406), A029404741 (SEQ ID NO: 407), A029404740 (SEQ ID NO: 408), A029404739 (SEQ ID NO: 409), A029404738 (SEQ ID NO: 410), A029404737 (SEQ ID NO: 411), A029404736 (SEQ ID NO: 412), A029404735 (SEQ ID NO: 413), A029404734 (SEQ ID NO: 414), A029404733 (SEQ ID NO: 415), A029404732 (SEQ ID NO: 416), A029404731 (SEQ ID NO: 417), A029404730 (SEQ ID NO: 418), A029404729 (SEQ ID NO: 419), A029404728 (SEQ ID NO: 420), A029404727 (SEQ ID NO: 421), A029404726 (SEQ ID NO: 422), A029404725 (SEQ ID NO: 423), A029404724 (SEQ ID NO: 424), A029404021 (SEQ ID NO: 425), A029403946 (SEQ ID NO: 426), A029401645 (SEQ ID NO: 427), A029404719 (SEQ ID NO: 428), A029401673 (SEQ ID NO: 429), A029403862 (SEQ ID NO: 430), A029403861 (SEQ ID NO: 431), A029403860 (SEQ ID NO: 432), A029401711 (SEQ ID NO: 433), A029401701 (SEQ ID NO: 434), A029401698 (SEQ ID NO: 435), A029401691 (SEQ ID NO: 436), A029401687 (SEQ ID NO: 437), A029401686 (SEQ ID NO: 438), A029401684 (SEQ ID NO: 439), A029401680 (SEQ ID NO: 440), A029401675 (SEQ ID NO: 441), A029403865 (SEQ ID NO: 442), A029405190 (SEQ ID NO: 443), A029401672 (SEQ ID NO: 444), A029401670 (SEQ ID NO: 445), A029401669 (SEQ ID NO: 446), A029401667 (SEQ ID NO: 447), A029401665 (SEQ ID NO: 448), A029401664 (SEQ ID NO: 449), A029401663 (SEQ ID NO: 450), A029401662 (SEQ ID NO: 451), A029401658 (SEQ ID NO: 452), A029401649 (SEQ ID NO: 453), A029401648 (SEQ ID NO: 454), A029401646 (SEQ ID NO: 455), A029405139 (SEQ ID NO: 456), A029405189 (SEQ ID NO: 457), A029403866 (SEQ ID NO: 458), A029403871 (SEQ ID NO: 459), A029401674 (SEQ ID NO: 460), A029403864 (SEQ ID NO: 461), A029403941 (SEQ ID NO: 462), A029403940 (SEQ ID NO: 463), A029403939 (SEQ ID NO: 464), A029403937 (SEQ ID NO: 465), A029403935 (SEQ ID NO: 466), A029403934 (SEQ ID NO: 467), A029403925 (SEQ ID NO: 468), A029403916 (SEQ ID NO: 469), A029403912 (SEQ ID NO: 470), A029403910 (SEQ ID NO: 471), A029403904 (SEQ ID NO: 472), A029403903 (SEQ ID NO: 473), A029403896 (SEQ ID NO: 474), A029403895 (SEQ ID NO: 475), A029403894 (SEQ ID NO: 476), A029403892 (SEQ ID NO: 477), A029403889 (SEQ ID NO: 478), A029403888 (SEQ ID NO: 479), A029403884 (SEQ ID NO: 480), A029403880 (SEQ ID NO: 481), A029403879 (SEQ ID NO: 482), A029403875 (SEQ ID NO: 483), A029403874 (SEQ ID NO: 484), A029403873 (SEQ ID NO: 485), A029403872 (SEQ ID NO: 486), A029403870 (SEQ ID NO: 487), or A029405191 (SEQ ID NO: 488) (see Table A-2 and A-6).

The SEQ ID NOs for the CDR sequences referred to above are based on the CDR definition according to the AbM definition (see Table A-6). It is noted that the CDR sequences defined according to the Kabat definition, Chothia definition or IMGT definition can likewise be used. Accordingly, the ISVDs provided by the present technology, specifically binding to PD-1 as described above using the AbM definition, can be also described using the Kabat definition, Chothia definition or IMGT definition.

In one embodiment, the ISVD comprises CDRs (Kabat definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 1-488, preferably SEQ ID NOs: 402, 331, 332, 375, 212, 139, 238.

In one embodiment, the ISVD comprises CDRs (Chothia definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 1-488, preferably SEQ ID NOs: 402, 331, 332, 375, 212, 139, 238.

In one embodiment, the ISVD comprises CDRs (IMGT definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 1-488, preferably SEQ ID NOs: 402, 331, 332, 375, 212, 139, 238.

In another embodiment, the ISVD specifically binding to human PD-1 may have a sequence identity of more than 90%, such as more than 95% or even more than 99%, with SEQ ID NO: 402, wherein the CDRs are as defined above. In one embodiment, the ISVD specifically binding to PD-1 comprises or consists of the amino acid sequence of SEQ ID NO: 402.

In another embodiment, the ISVD specifically binding to human PD-1 may have a sequence identity of more than 90%, such as more than 95% or even more than 99%, with SEQ ID NO: 1, wherein the CDRs are as defined above. In one embodiment, the ISVD specifically binding to PD-1 comprises or consists of the amino acid sequence of SEQ ID NO: 1.

In another embodiment, the ISVD specifically binding to human PD-1 may have a sequence identity of more than 90%, such as more than 95% or even more than 99%, with SEQ ID NO: 2, wherein the CDRs are as defined above. In one embodiment, the ISVD specifically binding to PD-1 comprises or consists of the amino acid sequence of SEQ ID NO: 2.

In another embodiment, the ISVD specifically binding to human PD-1 may have a sequence Identity of more than 90%, such as more than 95% or even more than 99%, with SEQ ID NO: 3, wherein the CDRs are as defined above. In one embodiment, the ISVD specifically binding to PD-1 comprises or consists of the amino acid sequence of SEQ ID NO: 3.

In another embodiment, the ISVD specifically binding to human PD-1 may have a sequence identity of more than 90%, such as more than 95% or even more than 99%, with SEQ ID NO: 4, wherein the CDRs are as defined above. In one embodiment, the ISVD specifically binding to PD-1 comprises or consists of the amino acid sequence of SEQ ID NO: 4.

In another embodiment, the ISVD specifically binding to human PD-1 may have a sequence identity of more than 90%, such as more than 95% or even more than 99%, with SEQ ID NO: 5, wherein the CDRs are as defined above. In one embodiment, the ISVD specifically binding to PD-1 comprises or consists of the amino acid sequence of SEQ ID NO: 5.

In another embodiment, the ISVD specifically binding to human PD-1 may have a sequence identity of more than 90%, such as more than 95% or even more than 99%, with SEQ ID NO: 6, wherein the CDRs are as defined above. In one embodiment, the ISVD specifically binding to PD-1 comprises or consists of the amino acid sequence of SEQ ID NO: 6.

In another embodiment, the ISVD specifically binding to human PD-1 may have a sequence identity of more than 90%, such as more than 95% or even more than 99%, with SEQ ID NO: 7, wherein the CDRs are as defined above. In one embodiment, the ISVD specifically binding to PD-1 comprises or consists of the amino acid sequence of SEQ ID NO: 7.

In another embodiment, the ISVD specifically binding to human PD-1 may have a sequence identity of more than 90%, such as more than 95% or even more than 99%, with SEQ ID NO: 8, wherein the CDRs are as defined above. In one embodiment, the ISVD specifically binding to PD-1 comprises or consists of the amino acid sequence of SEQ ID NO: 8.

In another embodiment, the ISVD specifically binding to human PD-1 may have a sequence identity of more than 90%, such as more than 95% or even more than 99%, with SEQ ID NO: 9, wherein the CDRs are as defined above. In one embodiment, the ISVD specifically binding to PD-1 comprises or consists of the amino acid sequence of SEQ ID NO: 9.

In another embodiment, the ISVD specifically binding to human PD-1 may have a sequence identity of more than 90%, such as more than 95% or even more than 99%, with SEQ ID NO: 10, wherein the CDRs are as defined above. In one embodiment, the ISVD specifically binding to PD-1 comprises or consists of the amino acid sequence of SEQ ID NO: 10.

In another embodiment, the ISVD specifically binding to human PD-1 may have a sequence identity of more than 90%, such as more than 95% or even more than 99%, with SEQ ID NO: 11, wherein the CDRs are as defined above. In one embodiment, the ISVD specifically binding to PD-1 comprises or consists of the amino acid sequence of SEQ ID NO: 11.

In another embodiment, the ISVD specifically binding to human PD-1 may have a sequence identity of more than 90%, such as more than 95% or even more than 99%, with SEQ ID NO: 12, wherein the CDRs are as defined above. In one embodiment, the ISVD specifically binding to PD-1 comprises or consists of the amino acid sequence of SEQ ID NO: 12.

In another embodiment, the ISVD specifically binding to human PD-1 may have a sequence identity of more than 90%, such as more than 95% or even more than 99%, with SEQ ID NO: 13, wherein the CDRs are as defined above. In one embodiment, the ISVD specifically binding to PD-1 comprises or consists of the amino acid sequence of SEQ ID NO: 13.

In another embodiment, the ISVD specifically binding to human PD-1 may have a sequence identity of more than 90%, such as more than 95% or even more than 99%, with SEQ ID NO: 14, wherein the CDRs are as defined above. In one embodiment, the ISVD specifically binding to PD-1 comprises or consists of the amino acid sequence of SEQ ID NO: 14.

In another embodiment, the ISVD specifically binding to human PD-1 may have a sequence identity of more than 90%, such as more than 95% or even more than 99%, with SEQ ID NO: 15, wherein the CDRs are as defined above. In one embodiment, the ISVD specifically binding to PD-1 comprises or consists of the amino acid sequence of SEQ ID NO: 15.

In another embodiment, the ISVD specifically binding to human PD-1 may have a sequence identity of more than 90%, such as more than 95% or even more than 99%, with SEQ ID NO: 16, wherein the CDRs are as defined above. In one embodiment, the ISVD specifically binding to PD-1 comprises or consists of the amino acid sequence of SEQ ID NO: 16.

In another embodiment, the ISVD specifically binding to human PD-1 may have a sequence identity of more than 90%, such as more than 95% or even more than 99%, with SEQ ID NO: 17, wherein the CDRs are as defined above. In one embodiment, the ISVD specifically binding to PD-1 comprises or consists of the amino acid sequence of SEQ ID NO: 17.

In another embodiment, the ISVD specifically binding to human PD-1 may have a sequence identity of more than 90%, such as more than 95% or even more than 99%, with SEQ ID NO: 18, wherein the CDRs are as defined above. In one embodiment, the ISVD specifically binding to PD-1 comprises or consists of the amino acid sequence of SEQ ID NO: 18.

In another embodiment, the ISVD specifically binding to human PD-1 may have a sequence identity of more than 90%, such as more than 95% or even more than 99%, with SEQ ID NO: 19, wherein the CDRs are as defined above. In one embodiment, the ISVD specifically binding to PD-1 comprises or consists of the amino acid sequence of SEQ ID NO: 19.

In another embodiment, the ISVD specifically binding to human PD-1 may have a sequence identity of more than 90%, such as more than 95% or even more than 99%, with SEQ ID NO: 20, wherein the CDRs are as defined above. In one embodiment, the ISVD specifically binding to PD-1 comprises or consists of the amino acid sequence of SEQ ID NO: 20.

In another embodiment, the ISVD specifically binding to human PD-1 may have a sequence identity of more than 90%, such as more than 95% or even more than 99%, with SEQ ID NO: 21, wherein the CDRs are as defined above. In one embodiment, the ISVD specifically binding to PD-1 comprises or consists of the amino acid sequence of SEQ ID NO: 21.

In another embodiment, the ISVD specifically binding to human PD-1 may have a sequence identity of more than 90%, such as more than 95% or even more than 99%, with SEQ ID NO: 22, wherein the CDRs are as defined above. In one embodiment, the ISVD specifically binding to PD-1 comprises or consists of the amino acid sequence of SEQ ID NO: 22.

In another embodiment, the ISVD specifically binding to human PD-1 may have a sequence identity of more than 90%, such as more than 95% or even more than 99%, with SEQ ID NO: 23, wherein the CDRs are as defined above. In one embodiment, the ISVD specifically binding to PD-1 comprises or consists of the amino acid sequence of SEQ ID NO: 23.

In another embodiment, the ISVD specifically binding to human PD-1 may have a sequence identity of more than 90%, such as more than 95% or even more than 99%, with SEQ ID NO: 24, wherein the CDRs are as defined above. In one embodiment, the ISVD specifically binding to PD-1 comprises or consists of the amino acid sequence of SEQ ID NO: 24.

In another embodiment, the ISVD specifically binding to human PD-1 may have a sequence identity of more than 90%, such as more than 95% or even more than 99%, with SEQ ID NO: 25, wherein the CDRs are as defined above. In one embodiment, the ISVD specifically binding to PD-1 comprises or consists of the amino acid sequence of SEQ ID NO: 25.

In another embodiment, the ISVD specifically binding to human PD-1 may have a sequence identity of more than 90%, such as more than 95% or even more than 99%, with SEQ ID NO: 26, wherein the CDRs are as defined above. In one embodiment, the ISVD specifically binding to PD-1 comprises or consists of the amino acid sequence of SEQ ID NO: 26.

In another embodiment, the ISVD specifically binding to human PD-1 may have a sequence identity of more than 90%, such as more than 95% or even more than 99%, with SEQ ID NO: 27, wherein the CDRs are as defined above. In one embodiment, the ISVD specifically binding to PD-1 comprises or consists of the amino acid sequence of SEQ ID NO: 27.

In another embodiment, the ISVD specifically binding to human PD-1 may have a sequence identity of more than 90%, such as more than 95% or even more than 99%, with SEQ ID NO: 28, wherein the CDRs are as defined above. In one embodiment, the ISVD specifically binding to PD-1 comprises or consists of the amino acid sequence of SEQ ID NO: 28.

In another embodiment, the ISVD specifically binding to human PD-1 may have a sequence identity of more than 90%, such as more than 95% or even more than 99%, with SEQ ID NO: 29, wherein the CDRs are as defined above. In one embodiment, the ISVD specifically binding to PD-1 comprises or consists of the amino acid sequence of SEQ ID NO: 29.

In another embodiment, the ISVD specifically binding to human PD-1 may have a sequence identity of more than 90%, such as more than 95% or even more than 99%, with SEQ ID NO: 30, wherein the CDRs are as defined above. In one embodiment, the ISVD specifically binding to PD-1 comprises or consists of the amino acid sequence of SEQ ID NO: 30.

In another embodiment, the ISVD specifically binding to human PD-1 may have a sequence identity of more than 90%, such as more than 95% or even more than 99%, with SEQ ID NO: 31, wherein the CDRs are as defined above. In one embodiment, the ISVD specifically binding to PD-1 comprises or consists of the amino acid sequence of SEQ ID NO: 31.

In another embodiment, the ISVD specifically binding to human PD-1 may have a sequence identity of more than 90%, such as more than 95% or even more than 99%, with SEQ ID NO: 32, wherein the CDRs are as defined above. In one embodiment, the ISVD specifically binding to PD-1 comprises or consists of the amino acid sequence of SEQ ID NO: 32.

In another embodiment, the ISVD specifically binding to human PD-1 may have a sequence identity of more than 90%, such as more than 95% or even more than 99%, with SEQ ID NO: 33, wherein the CDRs are as defined above. In one embodiment, the ISVD specifically binding to PD-1 comprises or consists of the amino acid sequence of SEQ ID NO: 33.

In another embodiment, the ISVD specifically binding to human PD-1 may have a sequence identity of more than 90%, such as more than 95% or even more than 99%, with SEQ ID NO: 34, wherein the CDRs are as defined above. In one embodiment, the ISVD specifically binding to PD-1 comprises or consists of the amino acid sequence of SEQ ID NO: 34.

In another embodiment, the ISVD specifically binding to human PD-1 may have a sequence identity of more than 90%, such as more than 95% or even more than 99%, with SEQ ID NO: 35, wherein the CDRs are as defined above. In one embodiment, the ISVD specifically binding to PD-1 comprises or consists of the amino acid sequence of SEQ ID NO: 35.

In another embodiment, the ISVD specifically binding to human PD-1 may have a sequence identity of more than 90%, such as more than 95% or even more than 99%, with SEQ ID NO: 36, wherein the CDRs are as defined above. In one embodiment, the ISVD specifically binding to PD-1 comprises or consists of the amino acid sequence of SEQ ID NO: 36.

In another embodiment, the ISVD specifically binding to human PD-1 may have a sequence identity of more than 90%, such as more than 95% or even more than 99%, with SEQ ID NO: 37, wherein the CDRs are as defined above. In one embodiment, the ISVD specifically binding to PD-1 comprises or consists of the amino acid sequence of SEQ ID NO: 37.

In another embodiment, the ISVD specifically binding to human PD-1 may have a sequence identity of more than 90%, such as more than 95% or even more than 99%, with SEQ ID NO: 38, wherein the CDRs are as defined above. In one embodiment, the ISVD specifically binding to PD-1 comprises or consists of the amino acid sequence of SEQ ID NO: 38.

In another embodiment, the ISVD specifically binding to human PD-1 may have a sequence identity of more than 90%, such as more than 95% or even more than 99%, with SEQ ID NO: 39, wherein the CDRs are as defined above. In one embodiment, the ISVD specifically binding to PD-1 comprises or consists of the amino acid sequence of SEQ ID NO: 39.

In another embodiment, the ISVD specifically binding to human PD-1 may have a sequence identity of more than 90%, such as more than 95% or even more than 99%, with SEQ ID NO: 40, wherein the CDRs are as defined above. In one embodiment, the ISVD specifically binding to PD-1 comprises or consists of the amino acid sequence of SEQ ID NO: 40.

In another embodiment, the ISVD specifically binding to human PD-1 may have a sequence identity of more than 90%, such as more than 95% or even more than 99%, with SEQ ID NO: 41, wherein the CDRs are as defined above. In one embodiment, the ISVD specifically binding to PD-1 comprises or consists of the amino acid sequence of SEQ ID NO: 41.

In another embodiment, the ISVD specifically binding to human PD-1 may have a sequence identity of more than 90%, such as more than 95% or even more than 99%, with SEQ ID NO: 42, wherein the CDRs are as defined above. In one embodiment, the ISVD specifically binding to PD-1 comprises or consists of the amino acid sequence of SEQ ID NO: 42.

In another embodiment, the ISVD specifically binding to human PD-1 may have a sequence identity of more than 90%, such as more than 95% or even more than 99%, with SEQ ID NO: 43, wherein the CDRs are as defined above. In one embodiment, the ISVD specifically binding to PD-1 comprises or consists of the amino acid sequence of SEQ ID NO: 43.

In another embodiment, the ISVD specifically binding to human PD-1 may have a sequence identity of more than 90%, such as more than 95% or even more than 99%, with SEQ ID NO: 44, wherein the CDRs are as defined above. In one embodiment, the ISVD specifically binding to PD-1 comprises or consists of the amino acid sequence of SEQ ID NO: 44.

In another embodiment, the ISVD specifically binding to human PD-1 may have a sequence identity of more than 90%, such as more than 95% or even more than 99%, with SEQ ID NO: 45, wherein the CDRs are as defined above. In one embodiment, the ISVD specifically binding to PD-1 comprises or consists of the amino acid sequence of SEQ ID NO: 45.

In another embodiment, the ISVD specifically binding to human PD-1 may have a sequence identity of more than 90%, such as more than 95% or even more than 99%, with SEQ ID NO: 46, wherein the CDRs are as defined above. In one embodiment, the ISVD specifically binding to PD-1 comprises or consists of the amino acid sequence of SEQ ID NO: 46.

In another embodiment, the ISVD specifically binding to human PD-1 may have a sequence identity of more than 90%, such as more than 95% or even more than 99%, with SEQ ID NO: 47, wherein the CDRs are as defined above. In one embodiment, the ISVD specifically binding to PD-1 comprises or consists of the amino acid sequence of SEQ ID NO: 47.

In another embodiment, the ISVD specifically binding to human PD-1 may have a sequence identity of more than 90%, such as more than 95% or even more than 99%, with SEQ ID NO: 48, wherein the CDRs are as defined above. In one embodiment, the ISVD specifically binding to PD-1 comprises or consists of the amino acid sequence of SEQ ID NO: 48.

In another embodiment, the ISVD specifically binding to human PD-1 may have a sequence identity of more than 90%, such as more than 95% or even more than 99%, with SEQ ID NO: 49, wherein the CDRs are as defined above. In one embodiment, the ISVD specifically binding to PD-1 comprises or consists of the amino acid sequence of SEQ ID NO: 49.

In another embodiment, the ISVD specifically binding to human PD-1 may have a sequence identity of more than 90%, such as more than 95% or even more than 99%, with SEQ ID NO: 50, wherein the CDRs are as defined above. In one embodiment, the ISVD specifically binding to PD-1 comprises or consists of the amino acid sequence of SEQ ID NO: 50.

In another embodiment, the ISVD specifically binding to human PD-1 may have a sequence identity of more than 90%, such as more than 95% or even more than 99%, with SEQ ID NO: 51, wherein the CDRs are as defined above. In one embodiment, the ISVD specifically binding to PD-1 comprises or consists of the amino acid sequence of SEQ ID NO: 51.

In another embodiment, the ISVD specifically binding to human PD-1 may have a sequence identity of more than 90%, such as more than 95% or even more than 99%, with SEQ ID NO: 52, wherein the CDRs are as defined above. In one embodiment, the ISVD specifically binding to PD-1 comprises or consists of the amino acid sequence of SEQ ID NO: 52.

In another embodiment, the ISVD specifically binding to human PD-1 may have a sequence identity of more than 90%, such as more than 95% or even more than 99%, with SEQ ID NO: 53, wherein the CDRs are as defined above. In one embodiment, the ISVD specifically binding to PD-1 comprises or consists of the amino acid sequence of SEQ ID NO: 53.

In another embodiment, the ISVD specifically binding to human PD-1 may have a sequence identity of more than 90%, such as more than 95% or even more than 99%, with SEQ ID NO: 54, wherein the CDRs are as defined above. In one embodiment, the ISVD specifically binding to PD-1 comprises or consists of the amino acid sequence of SEQ ID NO: 54.

In another embodiment, the ISVD specifically binding to human PD-1 may have a sequence identity of more than 90%, such as more than 95% or even more than 99%, with SEQ ID NO: 55, wherein the CDRs are as defined above. In one embodiment, the ISVD specifically binding to PD-1 comprises or consists of the amino acid sequence of SEQ ID NO: 55.

In another embodiment, the ISVD specifically binding to human PD-1 may have a sequence identity of more than 90%, such as more than 95% or even more than 99%, with SEQ ID NO: 56, wherein the CDRs are as defined above. In one embodiment, the ISVD specifically binding to PD-1 comprises or consists of the amino acid sequence of SEQ ID NO: 56.

In another embodiment, the ISVD specifically binding to human PD-1 may have a sequence identity of more than 90%, such as more than 95% or even more than 99%, with SEQ ID NO: 57, wherein the CDRs are as defined above. In one embodiment, the ISVD specifically binding to PD-1 comprises or consists of the amino acid sequence of SEQ ID NO: 57.

In another embodiment, the ISVD specifically binding to human PD-1 may have a sequence identity of more than 90%, such as more than 95% or even more than 99%, with SEQ ID NO: 58, wherein the CDRs are as defined above. In one embodiment, the ISVD specifically binding to PD-1 comprises or consists of the amino acid sequence of SEQ ID NO: 58.

In another embodiment, the ISVD specifically binding to human PD-1 may have a sequence identity of more than 90%, such as more than 95% or even more than 99%, with SEQ ID NO: 59, wherein the CDRs are as defined above. In one embodiment, the ISVD specifically binding to PD-1 comprises or consists of the amino acid sequence of SEQ ID NO: 59.

In another embodiment, the ISVD specifically binding to human PD-1 may have a sequence identity of more than 90%, such as more than 95% or even more than 99%, with SEQ ID NO: 60, wherein the CDRs are as defined above. In one embodiment, the ISVD specifically binding to PD-1 comprises or consists of the amino acid sequence of SEQ ID NO: 60.

In another embodiment, the ISVD specifically binding to human PD-1 may have a sequence identity of more than 90%, such as more than 95% or even more than 99%, with SEQ ID NO: 61, wherein the CDRs are as defined above. In one embodiment, the ISVD specifically binding to PD-1 comprises or consists of the amino acid sequence of SEQ ID NO: 61.

In another embodiment, the ISVD specifically binding to human PD-1 may have a sequence identity of more than 90%, such as more than 95% or even more than 99%, with SEQ ID NO: 62, wherein the CDRs are as defined above. In one embodiment, the ISVD specifically binding to PD-1 comprises or consists of the amino acid sequence of SEQ ID NO: 62.

In another embodiment, the ISVD specifically binding to human PD-1 may have a sequence identity of more than 90%, such as more than 95% or even more than 99%, with SEQ ID NO: 63, wherein the CDRs are as defined above. In one embodiment, the ISVD specifically binding to PD-1 comprises or consists of the amino acid sequence of SEQ ID NO: 63.

In another embodiment, the ISVD specifically binding to human PD-1 may have a sequence identity of more than 90%, such as more than 95% or even more than 99%, with SEQ ID NO: 64, wherein the CDRs are as defined above. In one embodiment, the ISVD specifically binding to PD-1 comprises or consists of the amino acid sequence of SEQ ID NO: 64.

In another embodiment, the ISVD specifically binding to human PD-1 may have a sequence identity of more than 90%, such as more than 95% or even more than 99%, with SEQ ID NO: 65, wherein the CDRs are as defined above. In one embodiment, the ISVD specifically binding to PD-1 comprises or consists of the amino acid sequence of SEQ ID NO: 65.

In another embodiment, the ISVD specifically binding to human PD-1 may have a sequence identity of more than 90%, such as more than 95% or even more than 99%, with SEQ ID NO: 66, wherein the CDRs are as defined above. In one embodiment, the ISVD specifically binding to PD-1 comprises or consists of the amino acid sequence of SEQ ID NO: 66.

In another embodiment, the ISVD specifically binding to human PD-1 may have a sequence identity of more than 90%, such as more than 95% or even more than 99%, with SEQ ID NO: 67, wherein the CDRs are as defined above. In one embodiment, the ISVD specifically binding to PD-1 comprises or consists of the amino acid sequence of SEQ ID NO: 67.

In another embodiment, the ISVD specifically binding to human PD-1 may have a sequence identity of more than 90%, such as more than 95% or even more than 99%, with SEQ ID NO: 68, wherein the CDRs are as defined above. In one embodiment, the ISVD specifically binding to PD-1 comprises or consists of the amino acid sequence of SEQ ID NO: 68.

In another embodiment, the ISVD specifically binding to human PD-1 may have a sequence identity of more than 90%, such as more than 95% or even more than 99%, with SEQ ID NO: 69, wherein the CDRs are as defined above. In one embodiment, the ISVD specifically binding to PD-1 comprises or consists of the amino acid sequence of SEQ ID NO: 69.

In another embodiment, the ISVD specifically binding to human PD-1 may have a sequence identity of more than 90%, such as more than 95% or even more than 99%, with SEQ ID NO: 70, wherein the CDRs are as defined above. In one embodiment, the ISVD specifically binding to PD-1 comprises or consists of the amino acid sequence of SEQ ID NO: 70.

In another embodiment, the ISVD specifically binding to human PD-1 may have a sequence identity of more than 90%, such as more than 95% or even more than 99%, with SEQ ID NO: 71, wherein the CDRs are as defined above. In one embodiment, the ISVD specifically binding to PD-1 comprises or consists of the amino acid sequence of SEQ ID NO: 71.

In another embodiment, the ISVD specifically binding to human PD-1 may have a sequence identity of more than 90%, such as more than 95% or even more than 99%, with SEQ ID NO: 72, wherein the CDRs are as defined above. In one embodiment, the ISVD specifically binding to PD-1 comprises or consists of the amino acid sequence of SEQ ID NO: 72.

In another embodiment, the ISVD specifically binding to human PD-1 may have a sequence identity of more than 90%, such as more than 95% or even more than 99%, with SEQ ID NO: 73, wherein the CDRs are as defined above. In one embodiment, the ISVD specifically binding to PD-1 comprises or consists of the amino acid sequence of SEQ ID NO: 73.

In another embodiment, the ISVD specifically binding to human PD-1 may have a sequence identity of more than 90%, such as more than 95% or even more than 99%, with SEQ ID NO: 74, wherein the CDRs are as defined above. In one embodiment, the ISVD specifically binding to PD-1 comprises or consists of the amino acid sequence of SEQ ID NO: 74.

In another embodiment, the ISVD specifically binding to human PD-1 may have a sequence identity of more than 90%, such as more than 95% or even more than 99%, with SEQ ID NO: 75, wherein the CDRs are as defined above. In one embodiment, the ISVD specifically binding to PD-1 comprises or consists of the amino acid sequence of SEQ ID NO: 75.

In another embodiment, the ISVD specifically binding to human PD-1 may have a sequence identity of more than 90%, such as more than 95% or even more than 99%, with SEQ ID NO: 76, wherein the CDRs are as defined above. In one embodiment, the ISVD specifically binding to PD-1 comprises or consists of the amino acid sequence of SEQ ID NO: 76.

In another embodiment, the ISVD specifically binding to human PD-1 may have a sequence identity of more than 90%, such as more than 95% or even more than 99%, with SEQ ID NO: 77, wherein the CDRs are as defined above. In one embodiment, the ISVD specifically binding to PD-1 comprises or consists of the amino acid sequence of SEQ ID NO: 77.

In another embodiment, the ISVD specifically binding to human PD-1 may have a sequence identity of more than 90%, such as more than 95% or even more than 99%, with SEQ ID NO: 78, wherein the CDRs are as defined above. In one embodiment, the ISVD specifically binding to PD-1 comprises or consists of the amino acid sequence of SEQ ID NO: 78.

In another embodiment, the ISVD specifically binding to human PD-1 may have a sequence identity of more than 90%, such as more than 95% or even more than 99%, with SEQ ID NO: 79, wherein the CDRs are as defined above. In one embodiment, the ISVD specifically binding to PD-1 comprises or consists of the amino acid sequence of SEQ ID NO: 79.

In another embodiment, the ISVD specifically binding to human PD-1 may have a sequence identity of more than 90%, such as more than 95% or even more than 99%, with SEQ ID NO: 80, wherein the CDRs are as defined above. In one embodiment, the ISVD specifically binding to PD-1 comprises or consists of the amino acid sequence of SEQ ID NO: 80.

In another embodiment, the ISVD specifically binding to human PD-1 may have a sequence identity of more than 90%, such as more than 95% or even more than 99%, with SEQ ID NO: 81, wherein the CDRs are as defined above. In one embodiment, the ISVD specifically binding to PD-1 comprises or consists of the amino acid sequence of SEQ ID NO: 81.

In another embodiment, the ISVD specifically binding to human PD-1 may have a sequence identity of more than 90%, such as more than 95% or even more than 99%, with SEQ ID NO: 82, wherein the CDRs are as defined above. In one embodiment, the ISVD specifically binding to PD-1 comprises or consists of the amino acid sequence of SEQ ID NO: 82.

In another embodiment, the ISVD specifically binding to human PD-1 may have a sequence identity of more than 90%, such as more than 95% or even more than 99%, with SEQ ID NO: 83, wherein the CDRs are as defined above. In one embodiment, the ISVD specifically binding to PD-1 comprises or consists of the amino acid sequence of SEQ ID NO: 83.

In another embodiment, the ISVD specifically binding to human PD-1 may have a sequence identity of more than 90%, such as more than 95% or even more than 99%, with SEQ ID NO: 84, wherein the CDRs are as defined above. In one embodiment, the ISVD specifically binding to PD-1 comprises or consists of the amino acid sequence of SEQ ID NO: 84.

In another embodiment, the ISVD specifically binding to human PD-1 may have a sequence identity of more than 90%, such as more than 95% or even more than 99%, with SEQ ID NO: 85, wherein the CDRs are as defined above. In one embodiment, the ISVD specifically binding to PD-1 comprises or consists of the amino acid sequence of SEQ ID NO: 85.

In another embodiment, the ISVD specifically binding to human PD-1 may have a sequence identity of more than 90%, such as more than 95% or even more than 99%, with SEQ ID NO: 86, wherein the CDRs are as defined above. In one embodiment, the ISVD specifically binding to PD-1 comprises or consists of the amino acid sequence of SEQ ID NO: 86.

In another embodiment, the ISVD specifically binding to human PD-1 may have a sequence identity of more than 90%, such as more than 95% or even more than 99%, with SEQ ID NO: 87, wherein the CDRs are as defined above. In one embodiment, the ISVD specifically binding to PD-1 comprises or consists of the amino acid sequence of SEQ ID NO: 87.

In another embodiment, the ISVD specifically binding to human PD-1 may have a sequence identity of more than 90%, such as more than 95% or even more than 99%, with SEQ ID NO: 88, wherein the CDRs are as defined above. In one embodiment, the ISVD specifically binding to PD-1 comprises or consists of the amino acid sequence of SEQ ID NO: 88.

In another embodiment, the ISVD specifically binding to human PD-1 may have a sequence identity of more than 90%, such as more than 95% or even more than 99%, with SEQ ID NO: 89, wherein the CDRs are as defined above. In one embodiment, the ISVD specifically binding to PD-1 comprises or consists of the amino acid sequence of SEQ ID NO: 89.

In another embodiment, the ISVD specifically binding to human PD-1 may have a sequence identity of more than 90%, such as more than 95% or even more than 99%, with SEQ ID NO: 90, wherein the CDRs are as defined above. In one embodiment, the ISVD specifically binding to PD-1 comprises or consists of the amino acid sequence of SEQ ID NO: 90.

In another embodiment, the ISVD specifically binding to human PD-1 may have a sequence identity of more than 90%, such as more than 95% or even more than 99%, with SEQ ID NO: 91, wherein the CDRs are as defined above. In one embodiment, the ISVD specifically binding to PD-1 comprises or consists of the amino acid sequence of SEQ ID NO: 91.

In another embodiment, the ISVD specifically binding to human PD-1 may have a sequence identity of more than 90%, such as more than 95% or even more than 99%, with SEQ ID NO: 92, wherein the CDRs are as defined above. In one embodiment, the ISVD specifically binding to PD-1 comprises or consists of the amino acid sequence of SEQ ID NO: 92.

In another embodiment, the ISVD specifically binding to human PD-1 may have a sequence identity of more than 90%, such as more than 95% or even more than 99%, with SEQ ID NO: 93, wherein the CDRs are as defined above. In one embodiment, the ISVD specifically binding to PD-1 comprises or consists of the amino acid sequence of SEQ ID NO: 93.

In another embodiment, the ISVD specifically binding to human PD-1 may have a sequence identity of more than 90%, such as more than 95% or even more than 99%, with SEQ ID NO: 94, wherein the CDRs are as defined above. In one embodiment, the ISVD specifically binding to PD-1 comprises or consists of the amino acid sequence of SEQ ID NO: 94.

In another embodiment, the ISVD specifically binding to human PD-1 may have a sequence identity of more than 90%, such as more than 95% or even more than 99%, with SEQ ID NO: 95, wherein the CDRs are as defined above. In one embodiment, the ISVD specifically binding to PD-1 comprises or consists of the amino acid sequence of SEQ ID NO: 95.

In another embodiment, the ISVD specifically binding to human PD-1 may have a sequence identity of more than 90%, such as more than 95% or even more than 99%, with SEQ ID NO: 96, wherein the CDRs are as defined above. In one embodiment, the ISVD specifically binding to PD-1 comprises or consists of the amino acid sequence of SEQ ID NO: 96.

In another embodiment, the ISVD specifically binding to human PD-1 may have a sequence identity of more than 90%, such as more than 95% or even more than 99%, with SEQ ID NO: 97, wherein the CDRs are as defined above. In one embodiment, the ISVD specifically binding to PD-1 comprises or consists of the amino acid sequence of SEQ ID NO: 97.

In another embodiment, the ISVD specifically binding to human PD-1 may have a sequence identity of more than 90%, such as more than 95% or even more than 99%, with SEQ ID NO: 98, wherein the CDRs are as defined above. In one embodiment, the ISVD specifically binding to PD-1 comprises or consists of the amino acid sequence of SEQ ID NO: 98.

In another embodiment, the ISVD specifically binding to human PD-1 may have a sequence identity of more than 90%, such as more than 95% or even more than 99%, with SEQ ID NO: 99, wherein the CDRs are as defined above. In one embodiment, the ISVD specifically binding to PD-1 comprises or consists of the amino acid sequence of SEQ ID NO: 99.

In another embodiment, the ISVD specifically binding to human PD-1 may have a sequence identity of more than 90%, such as more than 95% or even more than 99%, with SEQ ID NO: 100, wherein the CDRs are as defined above. In one embodiment, the ISVD specifically binding to PD-1 comprises or consists of the amino acid sequence of SEQ ID NO: 100.

In another embodiment, the ISVD specifically binding to human PD-1 may have a sequence identity of more than 90%, such as more than 95% or even more than 99%, with SEQ ID NO: 101, wherein the CDRs are as defined above. In one embodiment, the ISVD specifically binding to PD-1 comprises or consists of the amino acid sequence of SEQ ID NO: 101.

In another embodiment, the ISVD specifically binding to human PD-1 may have a sequence identity of more than 90%, such as more than 95% or even more than 99%, with SEQ ID NO: 102, wherein the CDRs are as defined above. In one embodiment, the ISVD specifically binding to PD-1 comprises or consists of the amino acid sequence of SEQ ID NO: 102.

In another embodiment, the ISVD specifically binding to human PD-1 may have a sequence Identity of more than 90%, such as more than 95% or even more than 99%, with SEQ ID NO: 103, wherein the CDRs are as defined above. In one embodiment, the ISVD specifically binding to PD-1 comprises or consists of the amino acid sequence of SEQ ID NO: 103.

In another embodiment, the ISVD specifically binding to human PD-1 may have a sequence identity of more than 90%, such as more than 95% or even more than 99%, with SEQ ID NO: 104, wherein the CDRs are as defined above. In one embodiment, the ISVD specifically binding to PD-1 comprises or consists of the amino acid sequence of SEQ ID NO: 104.

In another embodiment, the ISVD specifically binding to human PD-1 may have a sequence identity of more than 90%, such as more than 95% or even more than 99%, with SEQ ID NO: 105, wherein the CDRs are as defined above. In one embodiment, the ISVD specifically binding to PD-1 comprises or consists of the amino acid sequence of SEQ ID NO: 105.

In another embodiment, the ISVD specifically binding to human PD-1 may have a sequence identity of more than 90%, such as more than 95% or even more than 99%, with SEQ ID NO: 106, wherein the CDRs are as defined above. In one embodiment, the ISVD specifically binding to PD-1 comprises or consists of the amino acid sequence of SEQ ID NO: 106.

In another embodiment, the ISVD specifically binding to human PD-1 may have a sequence identity of more than 90%, such as more than 95% or even more than 99%, with SEQ ID NO: 107, wherein the CDRs are as defined above. In one embodiment, the ISVD specifically binding to PD-1 comprises or consists of the amino acid sequence of SEQ ID NO: 107.

In another embodiment, the ISVD specifically binding to human PD-1 may have a sequence identity of more than 90%, such as more than 95% or even more than 99%, with SEQ ID NO: 108, wherein the CDRs are as defined above. In one embodiment, the ISVD specifically binding to PD-1 comprises or consists of the amino acid sequence of SEQ ID NO: 108.

In another embodiment, the ISVD specifically binding to human PD-1 may have a sequence identity of more than 90%, such as more than 95% or even more than 99%, with SEQ ID NO: 109, wherein the CDRs are as defined above. In one embodiment, the ISVD specifically binding to PD-1 comprises or consists of the amino acid sequence of SEQ ID NO: 109.

In another embodiment, the ISVD specifically binding to human PD-1 may have a sequence identity of more than 90%, such as more than 95% or even more than 99%, with SEQ ID NO: 110, wherein the CDRs are as defined above. In one embodiment, the ISVD specifically binding to PD-1 comprises or consists of the amino acid sequence of SEQ ID NO: 110.

In another embodiment, the ISVD specifically binding to human PD-1 may have a sequence identity of more than 90%, such as more than 95% or even more than 99%, with SEQ ID NO: 111, wherein the CDRs are as defined above. In one embodiment, the ISVD specifically binding to PD-1 comprises or consists of the amino acid sequence of SEQ ID NO: 111.

In another embodiment, the ISVD specifically binding to human PD-1 may have a sequence identity of more than 90%, such as more than 95% or even more than 99%, with SEQ ID NO: 112, wherein the CDRs are as defined above. In one embodiment, the ISVD specifically binding to PD-1 comprises or consists of the amino acid sequence of SEQ ID NO: 112.

In another embodiment, the ISVD specifically binding to human PD-1 may have a sequence identity of more than 90%, such as more than 95% or even more than 99%, with SEQ ID NO: 113, wherein the CDRs are as defined above. In one embodiment, the ISVD specifically binding to PD-1 comprises or consists of the amino acid sequence of SEQ ID NO: 113.

In another embodiment, the ISVD specifically binding to human PD-1 may have a sequence identity of more than 90%, such as more than 95% or even more than 99%, with SEQ ID NO: 114, wherein the CDRs are as defined above. In one embodiment, the ISVD specifically binding to PD-1 comprises or consists of the amino acid sequence of SEQ ID NO: 114.

In another embodiment, the ISVD specifically binding to human PD-1 may have a sequence identity of more than 90%, such as more than 95% or even more than 99%, with SEQ ID NO: 115, wherein the CDRs are as defined above. In one embodiment, the ISVD specifically binding to PD-1 comprises or consists of the amino acid sequence of SEQ ID NO: 115.

In another embodiment, the ISVD specifically binding to human PD-1 may have a sequence identity of more than 90%, such as more than 95% or even more than 99%, with SEQ ID NO: 116, wherein the CDRs are as defined above. In one embodiment, the ISVD specifically binding to PD-1 comprises or consists of the amino acid sequence of SEQ ID NO: 116.

In another embodiment, the ISVD specifically binding to human PD-1 may have a sequence identity of more than 90%, such as more than 95% or even more than 99%, with SEQ ID NO: 117, wherein the CDRs are as defined above. In one embodiment, the ISVD specifically binding to PD-1 comprises or consists of the amino acid sequence of SEQ ID NO: 117.

In another embodiment, the ISVD specifically binding to human PD-1 may have a sequence identity of more than 90%, such as more than 95% or even more than 99%, with SEQ ID NO: 118, wherein the CDRs are as defined above. In one embodiment, the ISVD specifically binding to PD-1 comprises or consists of the amino acid sequence of SEQ ID NO: 118.

In another embodiment, the ISVD specifically binding to human PD-1 may have a sequence identity of more than 90%, such as more than 95% or even more than 99%, with SEQ ID NO: 119, wherein the CDRs are as defined above. In one embodiment, the ISVD specifically binding to PD-1 comprises or consists of the amino acid sequence of SEQ ID NO: 119.

In another embodiment, the ISVD specifically binding to human PD-1 may have a sequence identity of more than 90%, such as more than 95% or even more than 99%, with SEQ ID NO: 120, wherein the CDRs are as defined above. In one embodiment, the ISVD specifically binding to PD-1 comprises or consists of the amino acid sequence of SEQ ID NO: 120.

In another embodiment, the ISVD specifically binding to human PD-1 may have a sequence identity of more than 90%, such as more than 95% or even more than 99%, with SEQ ID NO: 121, wherein the CDRs are as defined above. In one embodiment, the ISVD specifically binding to PD-1 comprises or consists of the amino acid sequence of SEQ ID NO: 121.

In another embodiment, the ISVD specifically binding to human PD-1 may have a sequence identity of more than 90%, such as more than 95% or even more than 99%, with SEQ ID NO: 122, wherein the CDRs are as defined above. In one embodiment, the ISVD specifically binding to PD-1 comprises or consists of the amino acid sequence of SEQ ID NO: 122.

In another embodiment, the ISVD specifically binding to human PD-1 may have a sequence identity of more than 90%, such as more than 95% or even more than 99%, with SEQ ID NO: 123, wherein the CDRs are as defined above. In one embodiment, the ISVD specifically binding to PD-1 comprises or consists of the amino acid sequence of SEQ ID NO: 123.

In another embodiment, the ISVD specifically binding to human PD-1 may have a sequence identity of more than 90%, such as more than 95% or even more than 99%, with SEQ ID NO: 124, wherein the CDRs are as defined above. In one embodiment, the ISVD specifically binding to PD-1 comprises or consists of the amino acid sequence of SEQ ID NO: 124.

In another embodiment, the ISVD specifically binding to human PD-1 may have a sequence identity of more than 90%, such as more than 95% or even more than 99%, with SEQ ID NO: 125, wherein the CDRs are as defined above. In one embodiment, the ISVD specifically binding to PD-1 comprises or consists of the amino acid sequence of SEQ ID NO: 125.

In another embodiment, the ISVD specifically binding to human PD-1 may have a sequence identity of more than 90%, such as more than 95% or even more than 99%, with SEQ ID NO: 126, wherein the CDRs are as defined above. In one embodiment, the ISVD specifically binding to PD-1 comprises or consists of the amino acid sequence of SEQ ID NO: 126.

In another embodiment, the ISVD specifically binding to human PD-1 may have a sequence identity of more than 90%, such as more than 95% or even more than 99%, with SEQ ID NO: 127, wherein the CDRs are as defined above. In one embodiment, the ISVD specifically binding to PD-1 comprises or consists of the amino acid sequence of SEQ ID NO: 127.

In another embodiment, the ISVD specifically binding to human PD-1 may have a sequence identity of more than 90%, such as more than 95% or even more than 99%, with SEQ ID NO: 128, wherein the CDRs are as defined above. In one embodiment, the ISVD specifically binding to PD-1 comprises or consists of the amino acid sequence of SEQ ID NO: 128.

In another embodiment, the ISVD specifically binding to human PD-1 may have a sequence identity of more than 90%, such as more than 95% or even more than 99%, with SEQ ID NO: 129, wherein the CDRs are as defined above. In one embodiment, the ISVD specifically binding to PD-1 comprises or consists of the amino acid sequence of SEQ ID NO: 129.

In another embodiment, the ISVD specifically binding to human PD-1 may have a sequence identity of more than 90%, such as more than 95% or even more than 99%, with SEQ ID NO: 130, wherein the CDRs are as defined above. In one embodiment, the ISVD specifically binding to PD-1 comprises or consists of the amino acid sequence of SEQ ID NO: 130.

In another embodiment, the ISVD specifically binding to human PD-1 may have a sequence identity of more than 90%, such as more than 95% or even more than 99%, with SEQ ID NO: 131, wherein the CDRs are as defined above. In one embodiment, the ISVD specifically binding to PD-1 comprises or consists of the amino acid sequence of SEQ ID NO: 131.

In another embodiment, the ISVD specifically binding to human PD-1 may have a sequence identity of more than 90%, such as more than 95% or even more than 99%, with SEQ ID NO: 132, wherein the CDRs are as defined above. In one embodiment, the ISVD specifically binding to PD-1 comprises or consists of the amino acid sequence of SEQ ID NO: 132.

In another embodiment, the ISVD specifically binding to human PD-1 may have a sequence identity of more than 90%, such as more than 95% or even more than 99%, with SEQ ID NO: 133, wherein the CDRs are as defined above. In one embodiment, the ISVD specifically binding to PD-1 comprises or consists of the amino acid sequence of SEQ ID NO: 133.

In another embodiment, the ISVD specifically binding to human PD-1 may have a sequence identity of more than 90%, such as more than 95% or even more than 99%, with SEQ ID NO: 134, wherein the CDRs are as defined above. In one embodiment, the ISVD specifically binding to PD-1 comprises or consists of the amino acid sequence of SEQ ID NO: 134.

In another embodiment, the ISVD specifically binding to human PD-1 may have a sequence identity of more than 90%, such as more than 95% or even more than 99%, with SEQ ID NO: 135, wherein the CDRs are as defined above. In one embodiment, the ISVD specifically binding to PD-1 comprises or consists of the amino acid sequence of SEQ ID NO: 135.

In another embodiment, the ISVD specifically binding to human PD-1 may have a sequence identity of more than 90%, such as more than 95% or even more than 99%, with SEQ ID NO: 136, wherein the CDRs are as defined above. In one embodiment, the ISVD specifically binding to PD-1 comprises or consists of the amino acid sequence of SEQ ID NO: 136.

In another embodiment, the ISVD specifically binding to human PD-1 may have a sequence identity of more than 90%, such as more than 95% or even more than 99%, with SEQ ID NO: 137, wherein the CDRs are as defined above. In one embodiment, the ISVD specifically binding to PD-1 comprises or consists of the amino acid sequence of SEQ ID NO: 137.

In another embodiment, the ISVD specifically binding to human PD-1 may have a sequence identity of more than 90%, such as more than 95% or even more than 99%, with SEQ ID NO: 138, wherein the CDRs are as defined above. In one embodiment, the ISVD specifically binding to PD-1 comprises or consists of the amino acid sequence of SEQ ID NO: 138.

In another embodiment, the ISVD specifically binding to human PD-1 may have a sequence identity of more than 90%, such as more than 95% or even more than 99%, with SEQ ID NO: 139, wherein the CDRs are as defined above. In one embodiment, the ISVD specifically binding to PD-1 comprises or consists of the amino acid sequence of SEQ ID NO: 139.

In another embodiment, the ISVD specifically binding to human PD-1 may have a sequence identity of more than 90%, such as more than 95% or even more than 99%, with SEQ ID NO: 140, wherein the CDRs are as defined above. In one embodiment, the ISVD specifically binding to PD-1 comprises or consists of the amino acid sequence of SEQ ID NO: 140.

In another embodiment, the ISVD specifically binding to human PD-1 may have a sequence identity of more than 90%, such as more than 95% or even more than 99%, with SEQ ID NO: 141, wherein the CDRs are as defined above. In one embodiment, the ISVD specifically binding to PD-1 comprises or consists of the amino acid sequence of SEQ ID NO: 141.

In another embodiment, the ISVD specifically binding to human PD-1 may have a sequence identity of more than 90%, such as more than 95% or even more than 99%, with SEQ ID NO: 142, wherein the CDRs are as defined above. In one embodiment, the ISVD specifically binding to PD-1 comprises or consists of the amino acid sequence of SEQ ID NO: 142.

In another embodiment, the ISVD specifically binding to human PD-1 may have a sequence identity of more than 90%, such as more than 95% or even more than 99%, with SEQ ID NO: 143, wherein the CDRs are as defined above. In one embodiment, the ISVD specifically binding to PD-1 comprises or consists of the amino acid sequence of SEQ ID NO: 143.

In another embodiment, the ISVD specifically binding to human PD-1 may have a sequence identity of more than 90%, such as more than 95% or even more than 99%, with SEQ ID NO: 144, wherein the CDRs are as defined above. In one embodiment, the ISVD specifically binding to PD-1 comprises or consists of the amino acid sequence of SEQ ID NO: 144.

In another embodiment, the ISVD specifically binding to human PD-1 may have a sequence identity of more than 90%, such as more than 95% or even more than 99%, with SEQ ID NO: 145, wherein the CDRs are as defined above. In one embodiment, the ISVD specifically binding to PD-1 comprises or consists of the amino acid sequence of SEQ ID NO: 145.

In another embodiment, the ISVD specifically binding to human PD-1 may have a sequence identity of more than 90%, such as more than 95% or even more than 99%, with SEQ ID NO: 146, wherein the CDRs are as defined above. In one embodiment, the ISVD specifically binding to PD-1 comprises or consists of the amino acid sequence of SEQ ID NO: 146.

In another embodiment, the ISVD specifically binding to human PD-1 may have a sequence identity of more than 90%, such as more than 95% or even more than 99%, with SEQ ID NO: 147, wherein the CDRs are as defined above. In one embodiment, the ISVD specifically binding to PD-1 comprises or consists of the amino acid sequence of SEQ ID NO: 147.

In another embodiment, the ISVD specifically binding to human PD-1 may have a sequence identity of more than 90%, such as more than 95% or even more than 99%, with SEQ ID NO: 148, wherein the CDRs are as defined above. In one embodiment, the ISVD specifically binding to PD-1 comprises or consists of the amino acid sequence of SEQ ID NO: 148.

In another embodiment, the ISVD specifically binding to human PD-1 may have a sequence identity of more than 90%, such as more than 95% or even more than 99%, with SEQ ID NO: 149, wherein the CDRs are as defined above. In one embodiment, the ISVD specifically binding to PD-1 comprises or consists of the amino acid sequence of SEQ ID NO: 149.

In another embodiment, the ISVD specifically binding to human PD-1 may have a sequence identity of more than 90%, such as more than 95% or even more than 99%, with SEQ ID NO: 150, wherein the CDRs are as defined above. In one embodiment, the ISVD specifically binding to PD-1 comprises or consists of the amino acid sequence of SEQ ID NO: 150.

In another embodiment, the ISVD specifically binding to human PD-1 may have a sequence identity of more than 90%, such as more than 95% or even more than 99%, with SEQ ID NO: 151, wherein the CDRs are as defined above. In one embodiment, the ISVD specifically binding to PD-1 comprises or consists of the amino acid sequence of SEQ ID NO: 151.

In another embodiment, the ISVD specifically binding to human PD-1 may have a sequence identity of more than 90%, such as more than 95% or even more than 99%, with SEQ ID NO: 152, wherein the CDRs are as defined above. In one embodiment, the ISVD specifically binding to PD-1 comprises or consists of the amino acid sequence of SEQ ID NO: 152.

In another embodiment, the ISVD specifically binding to human PD-1 may have a sequence identity of more than 90%, such as more than 95% or even more than 99%, with SEQ ID NO: 153, wherein the CDRs are as defined above. In one embodiment, the ISVD specifically binding to PD-1 comprises or consists of the amino acid sequence of SEQ ID NO: 153.

In another embodiment, the ISVD specifically binding to human PD-1 may have a sequence identity of more than 90%, such as more than 95% or even more than 99%, with SEQ ID NO: 154, wherein the CDRs are as defined above. In one embodiment, the ISVD specifically binding to PD-1 comprises or consists of the amino acid sequence of SEQ ID NO: 154.

In another embodiment, the ISVD specifically binding to human PD-1 may have a sequence identity of more than 90%, such as more than 95% or even more than 99%, with SEQ ID NO: 155, wherein the CDRs are as defined above. In one embodiment, the ISVD specifically binding to PD-1 comprises or consists of the amino acid sequence of SEQ ID NO: 155.

In another embodiment, the ISVD specifically binding to human PD-1 may have a sequence identity of more than 90%, such as more than 95% or even more than 99%, with SEQ ID NO: 156, wherein the CDRs are as defined above. In one embodiment, the ISVD specifically binding to PD-1 comprises or consists of the amino acid sequence of SEQ ID NO: 156.

In another embodiment, the ISVD specifically binding to human PD-1 may have a sequence identity of more than 90%, such as more than 95% or even more than 99%, with SEQ ID NO: 157, wherein the CDRs are as defined above. In one embodiment, the ISVD specifically binding to PD-1 comprises or consists of the amino acid sequence of SEQ ID NO: 157.

In another embodiment, the ISVD specifically binding to human PD-1 may have a sequence identity of more than 90%, such as more than 95% or even more than 99%, with SEQ ID NO: 158, wherein the CDRs are as defined above. In one embodiment, the ISVD specifically binding to PD-1 comprises or consists of the amino acid sequence of SEQ ID NO: 158.

In another embodiment, the ISVD specifically binding to human PD-1 may have a sequence identity of more than 90%, such as more than 95% or even more than 99%, with SEQ ID NO: 159, wherein the CDRs are as defined above. In one embodiment, the ISVD specifically binding to PD-1 comprises or consists of the amino acid sequence of SEQ ID NO: 159.

In another embodiment, the ISVD specifically binding to human PD-1 may have a sequence identity of more than 90%, such as more than 95% or even more than 99%, with SEQ ID NO: 160, wherein the CDRs are as defined above. In one embodiment, the ISVD specifically binding to PD-1 comprises or consists of the amino acid sequence of SEQ ID NO: 160.

In another embodiment, the ISVD specifically binding to human PD-1 may have a sequence identity of more than 90%, such as more than 95% or even more than 99%, with SEQ ID NO: 161, wherein the CDRs are as defined above. In one embodiment, the ISVD specifically binding to PD-1 comprises or consists of the amino acid sequence of SEQ ID NO: 161.

In another embodiment, the ISVD specifically binding to human PD-1 may have a sequence identity of more than 90%, such as more than 95% or even more than 99%, with SEQ ID NO: 162, wherein the CDRs are as defined above. In one embodiment, the ISVD specifically binding to PD-1 comprises or consists of the amino acid sequence of SEQ ID NO: 162.

In another embodiment, the ISVD specifically binding to human PD-1 may have a sequence identity of more than 90%, such as more than 95% or even more than 99%, with SEQ ID NO: 163, wherein the CDRs are as defined above. In one embodiment, the ISVD specifically binding to PD-1 comprises or consists of the amino acid sequence of SEQ ID NO: 163.

In another embodiment, the ISVD specifically binding to human PD-1 may have a sequence identity of more than 90%, such as more than 95% or even more than 99%, with SEQ ID NO: 164, wherein the CDRs are as defined above. In one embodiment, the ISVD specifically binding to PD-1 comprises or consists of the amino acid sequence of SEQ ID NO: 164.

In another embodiment, the ISVD specifically binding to human PD-1 may have a sequence identity of more than 90%, such as more than 95% or even more than 99%, with SEQ ID NO: 165, wherein the CDRs are as defined above. In one embodiment, the ISVD specifically binding to PD-1 comprises or consists of the amino acid sequence of SEQ ID NO: 165.

In another embodiment, the ISVD specifically binding to human PD-1 may have a sequence identity of more than 90%, such as more than 95% or even more than 99%, with SEQ ID NO: 166, wherein the CDRs are as defined above. In one embodiment, the ISVD specifically binding to PD-1 comprises or consists of the amino acid sequence of SEQ ID NO: 166.

In another embodiment, the ISVD specifically binding to human PD-1 may have a sequence identity of more than 90%, such as more than 95% or even more than 99%, with SEQ ID NO: 167, wherein the CDRs are as defined above. In one embodiment, the ISVD specifically binding to PD-1 comprises or consists of the amino acid sequence of SEQ ID NO: 167.

In another embodiment, the ISVD specifically binding to human PD-1 may have a sequence identity of more than 90%, such as more than 95% or even more than 99%, with SEQ ID NO: 168, wherein the CDRs are as defined above. In one embodiment, the ISVD specifically binding to PD-1 comprises or consists of the amino acid sequence of SEQ ID NO: 168.

In another embodiment, the ISVD specifically binding to human PD-1 may have a sequence identity of more than 90%, such as more than 95% or even more than 99%, with SEQ ID NO: 169, wherein the CDRs are as defined above. In one embodiment, the ISVD specifically binding to PD-1 comprises or consists of the amino acid sequence of SEQ ID NO: 169.

In another embodiment, the ISVD specifically binding to human PD-1 may have a sequence identity of more than 90%, such as more than 95% or even more than 99%, with SEQ ID NO: 170, wherein the CDRs are as defined above. In one embodiment, the ISVD specifically binding to PD-1 comprises or consists of the amino acid sequence of SEQ ID NO: 170.

In another embodiment, the ISVD specifically binding to human PD-1 may have a sequence identity of more than 90%, such as more than 95% or even more than 99%, with SEQ ID NO: 171, wherein the CDRs are as defined above. In one embodiment, the ISVD specifically binding to PD-1 comprises or consists of the amino acid sequence of SEQ ID NO: 171.

In another embodiment, the ISVD specifically binding to human PD-1 may have a sequence identity of more than 90%, such as more than 95% or even more than 99%, with SEQ ID NO: 172, wherein the CDRs are as defined above. In one embodiment, the ISVD specifically binding to PD-1 comprises or consists of the amino acid sequence of SEQ ID NO: 172.

In another embodiment, the ISVD specifically binding to human PD-1 may have a sequence identity of more than 90%, such as more than 95% or even more than 99%, with SEQ ID NO: 173, wherein the CDRs are as defined above. In one embodiment, the ISVD specifically binding to PD-1 comprises or consists of the amino acid sequence of SEQ ID NO: 173.

In another embodiment, the ISVD specifically binding to human PD-1 may have a sequence identity of more than 90%, such as more than 95% or even more than 99%, with SEQ ID NO: 174, wherein the CDRs are as defined above. In one embodiment, the ISVD specifically binding to PD-1 comprises or consists of the amino acid sequence of SEQ ID NO: 174.

In another embodiment, the ISVD specifically binding to human PD-1 may have a sequence identity of more than 90%, such as more than 95% or even more than 99%, with SEQ ID NO: 175, wherein the CDRs are as defined above. In one embodiment, the ISVD specifically binding to PD-1 comprises or consists of the amino acid sequence of SEQ ID NO: 175.

In another embodiment, the ISVD specifically binding to human PD-1 may have a sequence identity of more than 90%, such as more than 95% or even more than 99%, with SEQ ID NO: 176, wherein the CDRs are as defined above. In one embodiment, the ISVD specifically binding to PD-1 comprises or consists of the amino acid sequence of SEQ ID NO: 176.

In another embodiment, the ISVD specifically binding to human PD-1 may have a sequence identity of more than 90%, such as more than 95% or even more than 99%, with SEQ ID NO: 177, wherein the CDRs are as defined above. In one embodiment, the ISVD specifically binding to PD-1 comprises or consists of the amino acid sequence of SEQ ID NO: 177.

In another embodiment, the ISVD specifically binding to human PD-1 may have a sequence identity of more than 90%, such as more than 95% or even more than 99%, with SEQ ID NO: 178, wherein the CDRs are as defined above. In one embodiment, the ISVD specifically binding to PD-1 comprises or consists of the amino acid sequence of SEQ ID NO: 178.

In another embodiment, the ISVD specifically binding to human PD-1 may have a sequence identity of more than 90%, such as more than 95% or even more than 99%, with SEQ ID NO: 179, wherein the CDRs are as defined above. In one embodiment, the ISVD specifically binding to PD-1 comprises or consists of the amino acid sequence of SEQ ID NO: 179.

In another embodiment, the ISVD specifically binding to human PD-1 may have a sequence identity of more than 90%, such as more than 95% or even more than 99%, with SEQ ID NO: 180, wherein the CDRs are as defined above. In one embodiment, the ISVD specifically binding to PD-1 comprises or consists of the amino acid sequence of SEQ ID NO: 180.

In another embodiment, the ISVD specifically binding to human PD-1 may have a sequence identity of more than 90%, such as more than 95% or even more than 99%, with SEQ ID NO: 181, wherein the CDRs are as defined above. In one embodiment, the ISVD specifically binding to PD-1 comprises or consists of the amino acid sequence of SEQ ID NO: 181.

In another embodiment, the ISVD specifically binding to human PD-1 may have a sequence identity of more than 90%, such as more than 95% or even more than 99%, with SEQ ID NO: 182, wherein the CDRs are as defined above. In one embodiment, the ISVD specifically binding to PD-1 comprises or consists of the amino acid sequence of SEQ ID NO: 182.

In another embodiment, the ISVD specifically binding to human PD-1 may have a sequence identity of more than 90%, such as more than 95% or even more than 99%, with SEQ ID NO: 183, wherein the CDRs are as defined above. In one embodiment, the ISVD specifically binding to PD-1 comprises or consists of the amino acid sequence of SEQ ID NO: 183.

In another embodiment, the ISVD specifically binding to human PD-1 may have a sequence identity of more than 90%, such as more than 95% or even more than 99%, with SEQ ID NO: 184, wherein the CDRs are as defined above. In one embodiment, the ISVD specifically binding to PD-1 comprises or consists of the amino acid sequence of SEQ ID NO: 184.

In another embodiment, the ISVD specifically binding to human PD-1 may have a sequence identity of more than 90%, such as more than 95% or even more than 99%, with SEQ ID NO: 185, wherein the CDRs are as defined above. In one embodiment, the ISVD specifically binding to PD-1 comprises or consists of the amino acid sequence of SEQ ID NO: 185.

In another embodiment, the ISVD specifically binding to human PD-1 may have a sequence identity of more than 90%, such as more than 95% or even more than 99%, with SEQ ID NO: 186, wherein the CDRs are as defined above. In one embodiment, the ISVD specifically binding to PD-1 comprises or consists of the amino acid sequence of SEQ ID NO: 186.

In another embodiment, the ISVD specifically binding to human PD-1 may have a sequence identity of more than 90%, such as more than 95% or even more than 99%, with SEQ ID NO: 187, wherein the CDRs are as defined above. In one embodiment, the ISVD specifically binding to PD-1 comprises or consists of the amino acid sequence of SEQ ID NO: 187.

In another embodiment, the ISVD specifically binding to human PD-1 may have a sequence identity of more than 90%, such as more than 95% or even more than 99%, with SEQ ID NO: 188, wherein the CDRs are as defined above. In one embodiment, the ISVD specifically binding to PD-1 comprises or consists of the amino acid sequence of SEQ ID NO: 188.

In another embodiment, the ISVD specifically binding to human PD-1 may have a sequence identity of more than 90%, such as more than 95% or even more than 99%, with SEQ ID NO: 189, wherein the CDRs are as defined above. In one embodiment, the ISVD specifically binding to PD-1 comprises or consists of the amino acid sequence of SEQ ID NO: 189.

In another embodiment, the ISVD specifically binding to human PD-1 may have a sequence identity of more than 90%, such as more than 95% or even more than 99%, with SEQ ID NO: 190, wherein the CDRs are as defined above. In one embodiment, the ISVD specifically binding to PD-1 comprises or consists of the amino acid sequence of SEQ ID NO: 190.

In another embodiment, the ISVD specifically binding to human PD-1 may have a sequence identity of more than 90%, such as more than 95% or even more than 99%, with SEQ ID NO: 191, wherein the CDRs are as defined above. In one embodiment, the ISVD specifically binding to PD-1 comprises or consists of the amino acid sequence of SEQ ID NO: 191.

In another embodiment, the ISVD specifically binding to human PD-1 may have a sequence identity of more than 90%, such as more than 95% or even more than 99%, with SEQ ID NO: 192, wherein the CDRs are as defined above. In one embodiment, the ISVD specifically binding to PD-1 comprises or consists of the amino acid sequence of SEQ ID NO: 192.

In another embodiment, the ISVD specifically binding to human PD-1 may have a sequence identity of more than 90%, such as more than 95% or even more than 99%, with SEQ ID NO: 193, wherein the CDRs are as defined above. In one embodiment, the ISVD specifically binding to PD-1 comprises or consists of the amino acid sequence of SEQ ID NO: 193.

In another embodiment, the ISVD specifically binding to human PD-1 may have a sequence identity of more than 90%, such as more than 95% or even more than 99%, with SEQ ID NO: 194, wherein the CDRs are as defined above. In one embodiment, the ISVD specifically binding to PD-1 comprises or consists of the amino acid sequence of SEQ ID NO: 194.

In another embodiment, the ISVD specifically binding to human PD-1 may have a sequence identity of more than 90%, such as more than 95% or even more than 99%, with SEQ ID NO: 195, wherein the CDRs are as defined above. In one embodiment, the ISVD specifically binding to PD-1 comprises or consists of the amino acid sequence of SEQ ID NO: 195.

In another embodiment, the ISVD specifically binding to human PD-1 may have a sequence identity of more than 90%, such as more than 95% or even more than 99%, with SEQ ID NO: 196, wherein the CDRs are as defined above. In one embodiment, the ISVD specifically binding to PD-1 comprises or consists of the amino acid sequence of SEQ ID NO: 196.

In another embodiment, the ISVD specifically binding to human PD-1 may have a sequence identity of more than 90%, such as more than 95% or even more than 99%, with SEQ ID NO: 197, wherein the CDRs are as defined above. In one embodiment, the ISVD specifically binding to PD-1 comprises or consists of the amino acid sequence of SEQ ID NO: 197.

In another embodiment, the ISVD specifically binding to human PD-1 may have a sequence identity of more than 90%, such as more than 95% or even more than 99%, with SEQ ID NO: 198, wherein the CDRs are as defined above. In one embodiment, the ISVD specifically binding to PD-1 comprises or consists of the amino acid sequence of SEQ ID NO: 198.

In another embodiment, the ISVD specifically binding to human PD-1 may have a sequence identity of more than 90%, such as more than 95% or even more than 99%, with SEQ ID NO: 199, wherein the CDRs are as defined above. In one embodiment, the ISVD specifically binding to PD-1 comprises or consists of the amino acid sequence of SEQ ID NO: 199.

In another embodiment, the ISVD specifically binding to human PD-1 may have a sequence identity of more than 90%, such as more than 95% or even more than 99%, with SEQ ID NO: 200, wherein the CDRs are as defined above. In one embodiment, the ISVD specifically binding to PD-1 comprises or consists of the amino acid sequence of SEQ ID NO: 200.

In another embodiment, the ISVD specifically binding to human PD-1 may have a sequence identity of more than 90%, such as more than 95% or even more than 99%, with SEQ ID NO: 201, wherein the CDRs are as defined above. In one embodiment, the ISVD specifically binding to PD-1 comprises or consists of the amino acid sequence of SEQ ID NO: 201.

In another embodiment, the ISVD specifically binding to human PD-1 may have a sequence identity of more than 90%, such as more than 95% or even more than 99%, with SEQ ID NO: 202, wherein the CDRs are as defined above. In one embodiment, the ISVD specifically binding to PD-1 comprises or consists of the amino acid sequence of SEQ ID NO: 202.

In another embodiment, the ISVD specifically binding to human PD-1 may have a sequence Identity of more than 90%, such as more than 95% or even more than 99%, with SEQ ID NO: 203, wherein the CDRs are as defined above. In one embodiment, the ISVD specifically binding to PD-1 comprises or consists of the amino acid sequence of SEQ ID NO: 203.

In another embodiment, the ISVD specifically binding to human PD-1 may have a sequence identity of more than 90%, such as more than 95% or even more than 99%, with SEQ ID NO: 204, wherein the CDRs are as defined above. In one embodiment, the ISVD specifically binding to PD-1 comprises or consists of the amino acid sequence of SEQ ID NO: 204.

In another embodiment, the ISVD specifically binding to human PD-1 may have a sequence identity of more than 90%, such as more than 95% or even more than 99%, with SEQ ID NO: 205, wherein the CDRs are as defined above. In one embodiment, the ISVD specifically binding to PD-1 comprises or consists of the amino acid sequence of SEQ ID NO: 205.

In another embodiment, the ISVD specifically binding to human PD-1 may have a sequence identity of more than 90%, such as more than 95% or even more than 99%, with SEQ ID NO: 206, wherein the CDRs are as defined above. In one embodiment, the ISVD specifically binding to PD-1 comprises or consists of the amino acid sequence of SEQ ID NO: 206.

In another embodiment, the ISVD specifically binding to human PD-1 may have a sequence identity of more than 90%, such as more than 95% or even more than 99%, with SEQ ID NO: 207, wherein the CDRs are as defined above. In one embodiment, the ISVD specifically binding to PD-1 comprises or consists of the amino acid sequence of SEQ ID NO: 207.

In another embodiment, the ISVD specifically binding to human PD-1 may have a sequence identity of more than 90%, such as more than 95% or even more than 99%, with SEQ ID NO: 208, wherein the CDRs are as defined above. In one embodiment, the ISVD specifically binding to PD-1 comprises or consists of the amino acid sequence of SEQ ID NO: 208.

In another embodiment, the ISVD specifically binding to human PD-1 may have a sequence identity of more than 90%, such as more than 95% or even more than 99%, with SEQ ID NO: 209, wherein the CDRs are as defined above. In one embodiment, the ISVD specifically binding to PD-1 comprises or consists of the amino acid sequence of SEQ ID NO: 209.

In another embodiment, the ISVD specifically binding to human PD-1 may have a sequence identity of more than 90%, such as more than 95% or even more than 99%, with SEQ ID NO: 2110, wherein the CDRs are as defined above. In one embodiment, the ISVD specifically binding to PD-1 comprises or consists of the amino acid sequence of SEQ ID NO: 210.

In another embodiment, the ISVD specifically binding to human PD-1 may have a sequence identity of more than 90%, such as more than 95% or even more than 99%, with SEQ ID NO: 211, wherein the CDRs are as defined above. In one embodiment, the ISVD specifically binding to PD-1 comprises or consists of the amino acid sequence of SEQ ID NO: 211.

In another embodiment, the ISVD specifically binding to human PD-1 may have a sequence identity of more than 90%, such as more than 95% or even more than 99%, with SEQ ID NO: 212, wherein the CDRs are as defined above. In one embodiment, the ISVD specifically binding to PD-1 comprises or consists of the amino acid sequence of SEQ ID NO: 212.

In another embodiment, the ISVD specifically binding to human PD-1 may have a sequence identity of more than 90%, such as more than 95% or even more than 99%, with SEQ ID NO: 213, wherein the CDRs are as defined above. In one embodiment, the ISVD specifically binding to PD-1 comprises or consists of the amino acid sequence of SEQ ID NO: 213.

In another embodiment, the ISVD specifically binding to human PD-1 may have a sequence identity of more than 90%, such as more than 95% or even more than 99%, with SEQ ID NO: 214, wherein the CDRs are as defined above. In one embodiment, the ISVD specifically binding to PD-1 comprises or consists of the amino acid sequence of SEQ ID NO: 214.

In another embodiment, the ISVD specifically binding to human PD-1 may have a sequence identity of more than 90%, such as more than 95% or even more than 99%, with SEQ ID NO: 215, wherein the CDRs are as defined above. In one embodiment, the ISVD specifically binding to PD-1 comprises or consists of the amino acid sequence of SEQ ID NO: 215.

In another embodiment, the ISVD specifically binding to human PD-1 may have a sequence identity of more than 90%, such as more than 95% or even more than 99%, with SEQ ID NO: 216, wherein the CDRs are as defined above. In one embodiment, the ISVD specifically binding to PD-1 comprises or consists of the amino acid sequence of SEQ ID NO: 216.

In another embodiment, the ISVD specifically binding to human PD-1 may have a sequence identity of more than 90%, such as more than 95% or even more than 99%, with SEQ ID NO: 217, wherein the CDRs are as defined above. In one embodiment, the ISVD specifically binding to PD-1 comprises or consists of the amino acid sequence of SEQ ID NO: 217.

In another embodiment, the ISVD specifically binding to human PD-1 may have a sequence identity of more than 90%, such as more than 95% or even more than 99%, with SEQ ID NO: 218, wherein the CDRs are as defined above. In one embodiment, the ISVD specifically binding to PD-1 comprises or consists of the amino acid sequence of SEQ ID NO: 218.

In another embodiment, the ISVD specifically binding to human PD-1 may have a sequence identity of more than 90%, such as more than 95% or even more than 99%, with SEQ ID NO: 219, wherein the CDRs are as defined above. In one embodiment, the ISVD specifically binding to PD-1 comprises or consists of the amino acid sequence of SEQ ID NO: 219.

In another embodiment, the ISVD specifically binding to human PD-1 may have a sequence identity of more than 90%, such as more than 95% or even more than 99%, with SEQ ID NO: 220, wherein the CDRs are as defined above. In one embodiment, the ISVD specifically binding to PD-1 comprises or consists of the amino acid sequence of SEQ ID NO: 220.

In another embodiment, the ISVD specifically binding to human PD-1 may have a sequence identity of more than 90%, such as more than 95% or even more than 99%, with SEQ ID NO: 221, wherein the CDRs are as defined above. In one embodiment, the ISVD specifically binding to PD-1 comprises or consists of the amino acid sequence of SEQ ID NO: 221.

In another embodiment, the ISVD specifically binding to human PD-1 may have a sequence identity of more than 90%, such as more than 95% or even more than 99%, with SEQ ID NO: 222, wherein the CDRs are as defined above. In one embodiment, the ISVD specifically binding to PD-1 comprises or consists of the amino acid sequence of SEQ ID NO: 222.

In another embodiment, the ISVD specifically binding to human PD-1 may have a sequence identity of more than 90%, such as more than 95% or even more than 99%, with SEQ ID NO: 223, wherein the CDRs are as defined above. In one embodiment, the ISVD specifically binding to PD-1 comprises or consists of the amino acid sequence of SEQ ID NO: 223.

In another embodiment, the ISVD specifically binding to human PD-1 may have a sequence identity of more than 90%, such as more than 95% or even more than 99%, with SEQ ID NO: 224, wherein the CDRs are as defined above. In one embodiment, the ISVD specifically binding to PD-1 comprises or consists of the amino acid sequence of SEQ ID NO: 224.

In another embodiment, the ISVD specifically binding to human PD-1 may have a sequence identity of more than 90%, such as more than 95% or even more than 99%, with SEQ ID NO: 225, wherein the CDRs are as defined above. In one embodiment, the ISVD specifically binding to PD-1 comprises or consists of the amino acid sequence of SEQ ID NO: 225.

In another embodiment, the ISVD specifically binding to human PD-1 may have a sequence identity of more than 90%, such as more than 95% or even more than 99%, with SEQ ID NO: 226, wherein the CDRs are as defined above. In one embodiment, the ISVD specifically binding to PD-1 comprises or consists of the amino acid sequence of SEQ ID NO: 226.

In another embodiment, the ISVD specifically binding to human PD-1 may have a sequence identity of more than 90%, such as more than 95% or even more than 99%, with SEQ ID NO: 227, wherein the CDRs are as defined above. In one embodiment, the ISVD specifically binding to PD-1 comprises or consists of the amino acid sequence of SEQ ID NO: 227.

In another embodiment, the ISVD specifically binding to human PD-1 may have a sequence identity of more than 90%, such as more than 95% or even more than 99%, with SEQ ID NO: 228, wherein the CDRs are as defined above. In one embodiment, the ISVD specifically binding to PD-1 comprises or consists of the amino acid sequence of SEQ ID NO: 228.

In another embodiment, the ISVD specifically binding to human PD-1 may have a sequence identity of more than 90%, such as more than 95% or even more than 99%, with SEQ ID NO: 229, wherein the CDRs are as defined above. In one embodiment, the ISVD specifically binding to PD-1 comprises or consists of the amino acid sequence of SEQ ID NO: 229.

In another embodiment, the ISVD specifically binding to human PD-1 may have a sequence identity of more than 90%, such as more than 95% or even more than 99%, with SEQ ID NO: 230, wherein the CDRs are as defined above. In one embodiment, the ISVD specifically binding to PD-1 comprises or consists of the amino acid sequence of SEQ ID NO: 230.

In another embodiment, the ISVD specifically binding to human PD-1 may have a sequence identity of more than 90%, such as more than 95% or even more than 99%, with SEQ ID NO: 231, wherein the CDRs are as defined above. In one embodiment, the ISVD specifically binding to PD-1 comprises or consists of the amino acid sequence of SEQ ID NO: 231.

In another embodiment, the ISVD specifically binding to human PD-1 may have a sequence identity of more than 90%, such as more than 95% or even more than 99%, with SEQ ID NO: 232, wherein the CDRs are as defined above. In one embodiment, the ISVD specifically binding to PD-1 comprises or consists of the amino acid sequence of SEQ ID NO: 232.

In another embodiment, the ISVD specifically binding to human PD-1 may have a sequence identity of more than 90%, such as more than 95% or even more than 99%, with SEQ ID NO: 233, wherein the CDRs are as defined above. In one embodiment, the ISVD specifically binding to PD-1 comprises or consists of the amino acid sequence of SEQ ID NO: 233.

In another embodiment, the ISVD specifically binding to human PD-1 may have a sequence identity of more than 90%, such as more than 95% or even more than 99%, with SEQ ID NO: 234, wherein the CDRs are as defined above. In one embodiment, the ISVD specifically binding to PD-1 comprises or consists of the amino acid sequence of SEQ ID NO: 234.

In another embodiment, the ISVD specifically binding to human PD-1 may have a sequence identity of more than 90%, such as more than 95% or even more than 99%, with SEQ ID NO: 235, wherein the CDRs are as defined above. In one embodiment, the ISVD specifically binding to PD-1 comprises or consists of the amino acid sequence of SEQ ID NO: 235.

In another embodiment, the ISVD specifically binding to human PD-1 may have a sequence identity of more than 90%, such as more than 95% or even more than 99%, with SEQ ID NO: 236, wherein the CDRs are as defined above. In one embodiment, the ISVD specifically binding to PD-1 comprises or consists of the amino acid sequence of SEQ ID NO: 236.

In another embodiment, the ISVD specifically binding to human PD-1 may have a sequence identity of more than 90%, such as more than 95% or even more than 99%, with SEQ ID NO: 237, wherein the CDRs are as defined above. In one embodiment, the ISVD specifically binding to PD-1 comprises or consists of the amino acid sequence of SEQ ID NO: 237.

In another embodiment, the ISVD specifically binding to human PD-1 may have a sequence identity of more than 90%, such as more than 95% or even more than 99%, with SEQ ID NO: 238, wherein the CDRs are as defined above. In one embodiment, the ISVD specifically binding to PD-1 comprises or consists of the amino acid sequence of SEQ ID NO: 238.

In another embodiment, the ISVD specifically binding to human PD-1 may have a sequence identity of more than 90%, such as more than 95% or even more than 99%, with SEQ ID NO: 239, wherein the CDRs are as defined above. In one embodiment, the ISVD specifically binding to PD-1 comprises or consists of the amino acid sequence of SEQ ID NO: 239.

In another embodiment, the ISVD specifically binding to human PD-1 may have a sequence identity of more than 90%, such as more than 95% or even more than 99%, with SEQ ID NO: 240, wherein the CDRs are as defined above. In one embodiment, the ISVD specifically binding to PD-1 comprises or consists of the amino acid sequence of SEQ ID NO: 240.

In another embodiment, the ISVD specifically binding to human PD-1 may have a sequence identity of more than 90%, such as more than 95% or even more than 99%, with SEQ ID NO: 241, wherein the CDRs are as defined above. In one embodiment, the ISVD specifically binding to PD-1 comprises or consists of the amino acid sequence of SEQ ID NO: 241.

In another embodiment, the ISVD specifically binding to human PD-1 may have a sequence identity of more than 90%, such as more than 95% or even more than 99%, with SEQ ID NO: 242, wherein the CDRs are as defined above. In one embodiment, the ISVD specifically binding to PD-1 comprises or consists of the amino acid sequence of SEQ ID NO: 242.

In another embodiment, the ISVD specifically binding to human PD-1 may have a sequence identity of more than 90%, such as more than 95% or even more than 99%, with SEQ ID NO: 243, wherein the CDRs are as defined above. In one embodiment, the ISVD specifically binding to PD-1 comprises or consists of the amino acid sequence of SEQ ID NO: 243.

In another embodiment, the ISVD specifically binding to human PD-1 may have a sequence identity of more than 90%, such as more than 95% or even more than 99%, with SEQ ID NO: 244, wherein the CDRs are as defined above. In one embodiment, the ISVD specifically binding to PD-1 comprises or consists of the amino acid sequence of SEQ ID NO: 244.

In another embodiment, the ISVD specifically binding to human PD-1 may have a sequence identity of more than 90%, such as more than 95% or even more than 99%, with SEQ ID NO: 245, wherein the CDRs are as defined above. In one embodiment, the ISVD specifically binding to PD-1 comprises or consists of the amino acid sequence of SEQ ID NO: 245.

In another embodiment, the ISVD specifically binding to human PD-1 may have a sequence identity of more than 90%, such as more than 95% or even more than 99%, with SEQ ID NO: 246, wherein the CDRs are as defined above. In one embodiment, the ISVD specifically binding to PD-1 comprises or consists of the amino acid sequence of SEQ ID NO: 246.

In another embodiment, the ISVD specifically binding to human PD-1 may have a sequence identity of more than 90%, such as more than 95% or even more than 99%, with SEQ ID NO: 247, wherein the CDRs are as defined above. In one embodiment, the ISVD specifically binding to PD-1 comprises or consists of the amino acid sequence of SEQ ID NO: 247.

In another embodiment, the ISVD specifically binding to human PD-1 may have a sequence identity of more than 90%, such as more than 95% or even more than 99%, with SEQ ID NO: 248, wherein the CDRs are as defined above. In one embodiment, the ISVD specifically binding to PD-1 comprises or consists of the amino acid sequence of SEQ ID NO: 248.

In another embodiment, the ISVD specifically binding to human PD-1 may have a sequence identity of more than 90%, such as more than 95% or even more than 99%, with SEQ ID NO: 249, wherein the CDRs are as defined above. In one embodiment, the ISVD specifically binding to PD-1 comprises or consists of the amino acid sequence of SEQ ID NO: 249.

In another embodiment, the ISVD specifically binding to human PD-1 may have a sequence identity of more than 90%, such as more than 95% or even more than 99%, with SEQ ID NO: 250, wherein the CDRs are as defined above. In one embodiment, the ISVD specifically binding to PD-1 comprises or consists of the amino acid sequence of SEQ ID NO: 250.

In another embodiment, the ISVD specifically binding to human PD-1 may have a sequence identity of more than 90%, such as more than 95% or even more than 99%, with SEQ ID NO: 251, wherein the CDRs are as defined above. In one embodiment, the ISVD specifically binding to PD-1 comprises or consists of the amino acid sequence of SEQ ID NO: 251.

In another embodiment, the ISVD specifically binding to human PD-1 may have a sequence identity of more than 90%, such as more than 95% or even more than 99%, with SEQ ID NO: 252, wherein the CDRs are as defined above. In one embodiment, the ISVD specifically binding to PD-1 comprises or consists of the amino acid sequence of SEQ ID NO: 252.

In another embodiment, the ISVD specifically binding to human PD-1 may have a sequence identity of more than 90%, such as more than 95% or even more than 99%, with SEQ ID NO: 253, wherein the CDRs are as defined above. In one embodiment, the ISVD specifically binding to PD-1 comprises or consists of the amino acid sequence of SEQ ID NO: 253.

In another embodiment, the ISVD specifically binding to human PD-1 may have a sequence identity of more than 90%, such as more than 95% or even more than 99%, with SEQ ID NO: 254, wherein the CDRs are as defined above. In one embodiment, the ISVD specifically binding to PD-1 comprises or consists of the amino acid sequence of SEQ ID NO: 254.

In another embodiment, the ISVD specifically binding to human PD-1 may have a sequence identity of more than 90%, such as more than 95% or even more than 99%, with SEQ ID NO: 255, wherein the CDRs are as defined above. In one embodiment, the ISVD specifically binding to PD-1 comprises or consists of the amino acid sequence of SEQ ID NO: 255.

In another embodiment, the ISVD specifically binding to human PD-1 may have a sequence identity of more than 90%, such as more than 95% or even more than 99%, with SEQ ID NO: 256, wherein the CDRs are as defined above. In one embodiment, the ISVD specifically binding to PD-1 comprises or consists of the amino acid sequence of SEQ ID NO: 256.

In another embodiment, the ISVD specifically binding to human PD-1 may have a sequence identity of more than 90%, such as more than 95% or even more than 99%, with SEQ ID NO: 257, wherein the CDRs are as defined above. In one embodiment, the ISVD specifically binding to PD-1 comprises or consists of the amino acid sequence of SEQ ID NO: 257.

In another embodiment, the ISVD specifically binding to human PD-1 may have a sequence identity of more than 90%, such as more than 95% or even more than 99%, with SEQ ID NO: 258, wherein the CDRs are as defined above. In one embodiment, the ISVD specifically binding to PD-1 comprises or consists of the amino acid sequence of SEQ ID NO: 258.

In another embodiment, the ISVD specifically binding to human PD-1 may have a sequence identity of more than 90%, such as more than 95% or even more than 99%, with SEQ ID NO: 259, wherein the CDRs are as defined above. In one embodiment, the ISVD specifically binding to PD-1 comprises or consists of the amino acid sequence of SEQ ID NO: 259.

In another embodiment, the ISVD specifically binding to human PD-1 may have a sequence identity of more than 90%, such as more than 95% or even more than 99%, with SEQ ID NO: 260, wherein the CDRs are as defined above. In one embodiment, the ISVD specifically binding to PD-1 comprises or consists of the amino acid sequence of SEQ ID NO: 260.

In another embodiment, the ISVD specifically binding to human PD-1 may have a sequence identity of more than 90%, such as more than 95% or even more than 99%, with SEQ ID NO: 261, wherein the CDRs are as defined above. In one embodiment, the ISVD specifically binding to PD-1 comprises or consists of the amino acid sequence of SEQ ID NO: 261.

In another embodiment, the ISVD specifically binding to human PD-1 may have a sequence identity of more than 90%, such as more than 95% or even more than 99%, with SEQ ID NO: 262, wherein the CDRs are as defined above. In one embodiment, the ISVD specifically binding to PD-1 comprises or consists of the amino acid sequence of SEQ ID NO: 262.

In another embodiment, the ISVD specifically binding to human PD-1 may have a sequence identity of more than 90%, such as more than 95% or even more than 99%, with SEQ ID NO: 263, wherein the CDRs are as defined above. In one embodiment, the ISVD specifically binding to PD-1 comprises or consists of the amino acid sequence of SEQ ID NO: 263.

In another embodiment, the ISVD specifically binding to human PD-1 may have a sequence identity of more than 90%, such as more than 95% or even more than 99%, with SEQ ID NO: 264, wherein the CDRs are as defined above. In one embodiment, the ISVD specifically binding to PD-1 comprises or consists of the amino acid sequence of SEQ ID NO: 264.

In another embodiment, the ISVD specifically binding to human PD-1 may have a sequence identity of more than 90%, such as more than 95% or even more than 99%, with SEQ ID NO: 265, wherein the CDRs are as defined above. In one embodiment, the ISVD specifically binding to PD-1 comprises or consists of the amino acid sequence of SEQ ID NO: 265.

In another embodiment, the ISVD specifically binding to human PD-1 may have a sequence identity of more than 90%, such as more than 95% or even more than 99%, with SEQ ID NO: 266, wherein the CDRs are as defined above. In one embodiment, the ISVD specifically binding to PD-1 comprises or consists of the amino acid sequence of SEQ ID NO: 266.

In another embodiment, the ISVD specifically binding to human PD-1 may have a sequence identity of more than 90%, such as more than 95% or even more than 99%, with SEQ ID NO: 267, wherein the CDRs are as defined above. In one embodiment, the ISVD specifically binding to PD-1 comprises or consists of the amino acid sequence of SEQ ID NO: 267.

In another embodiment, the ISVD specifically binding to human PD-1 may have a sequence identity of more than 90%, such as more than 95% or even more than 99%, with SEQ ID NO: 268, wherein the CDRs are as defined above. In one embodiment, the ISVD specifically binding to PD-1 comprises or consists of the amino acid sequence of SEQ ID NO: 268.

In another embodiment, the ISVD specifically binding to human PD-1 may have a sequence identity of more than 90%, such as more than 95% or even more than 99%, with SEQ ID NO: 269, wherein the CDRs are as defined above. In one embodiment, the ISVD specifically binding to PD-1 comprises or consists of the amino acid sequence of SEQ ID NO: 269.

In another embodiment, the ISVD specifically binding to human PD-1 may have a sequence identity of more than 90%, such as more than 95% or even more than 99%, with SEQ ID NO: 270, wherein the CDRs are as defined above. In one embodiment, the ISVD specifically binding to PD-1 comprises or consists of the amino acid sequence of SEQ ID NO: 270.

In another embodiment, the ISVD specifically binding to human PD-1 may have a sequence identity of more than 90%, such as more than 95% or even more than 99%, with SEQ ID NO: 271, wherein the CDRs are as defined above. In one embodiment, the ISVD specifically binding to PD-1 comprises or consists of the amino acid sequence of SEQ ID NO: 271.

In another embodiment, the ISVD specifically binding to human PD-1 may have a sequence identity of more than 90%, such as more than 95% or even more than 99%, with SEQ ID NO: 272, wherein the CDRs are as defined above. In one embodiment, the ISVD specifically binding to PD-1 comprises or consists of the amino acid sequence of SEQ ID NO: 272.

In another embodiment, the ISVD specifically binding to human PD-1 may have a sequence identity of more than 90%, such as more than 95% or even more than 99%, with SEQ ID NO: 273, wherein the CDRs are as defined above. In one embodiment, the ISVD specifically binding to PD-1 comprises or consists of the amino acid sequence of SEQ ID NO: 273.

In another embodiment, the ISVD specifically binding to human PD-1 may have a sequence identity of more than 90%, such as more than 95% or even more than 99%, with SEQ ID NO: 274, wherein the CDRs are as defined above. In one embodiment, the ISVD specifically binding to PD-1 comprises or consists of the amino acid sequence of SEQ ID NO: 274.

In another embodiment, the ISVD specifically binding to human PD-1 may have a sequence identity of more than 90%, such as more than 95% or even more than 99%, with SEQ ID NO: 275, wherein the CDRs are as defined above. In one embodiment, the ISVD specifically binding to PD-1 comprises or consists of the amino acid sequence of SEQ ID NO: 275.

In another embodiment, the ISVD specifically binding to human PD-1 may have a sequence identity of more than 90%, such as more than 95% or even more than 99%, with SEQ ID NO: 276, wherein the CDRs are as defined above. In one embodiment, the ISVD specifically binding to PD-1 comprises or consists of the amino acid sequence of SEQ ID NO: 276.

In another embodiment, the ISVD specifically binding to human PD-1 may have a sequence identity of more than 90%, such as more than 95% or even more than 99%, with SEQ ID NO: 277, wherein the CDRs are as defined above. In one embodiment, the ISVD specifically binding to PD-1 comprises or consists of the amino acid sequence of SEQ ID NO: 277.

In another embodiment, the ISVD specifically binding to human PD-1 may have a sequence identity of more than 90%, such as more than 95% or even more than 99%, with SEQ ID NO: 278, wherein the CDRs are as defined above. In one embodiment, the ISVD specifically binding to PD-1 comprises or consists of the amino acid sequence of SEQ ID NO: 278.

In another embodiment, the ISVD specifically binding to human PD-1 may have a sequence identity of more than 90%, such as more than 95% or even more than 99%, with SEQ ID NO: 279, wherein the CDRs are as defined above. In one embodiment, the ISVD specifically binding to PD-1 comprises or consists of the amino acid sequence of SEQ ID NO: 279.

In another embodiment, the ISVD specifically binding to human PD-1 may have a sequence identity of more than 90%, such as more than 95% or even more than 99%, with SEQ ID NO: 280, wherein the CDRs are as defined above. In one embodiment, the ISVD specifically binding to PD-1 comprises or consists of the amino acid sequence of SEQ ID NO: 280.

In another embodiment, the ISVD specifically binding to human PD-1 may have a sequence identity of more than 90%, such as more than 95% or even more than 99%, with SEQ ID NO: 281, wherein the CDRs are as defined above. In one embodiment, the ISVD specifically binding to PD-1 comprises or consists of the amino acid sequence of SEQ ID NO: 281.

In another embodiment, the ISVD specifically binding to human PD-1 may have a sequence identity of more than 90%, such as more than 95% or even more than 99%, with SEQ ID NO: 282, wherein the CDRs are as defined above. In one embodiment, the ISVD specifically binding to PD-1 comprises or consists of the amino acid sequence of SEQ ID NO: 282.

In another embodiment, the ISVD specifically binding to human PD-1 may have a sequence identity of more than 90%, such as more than 95% or even more than 99%, with SEQ ID NO: 283, wherein the CDRs are as defined above. In one embodiment, the ISVD specifically binding to PD-1 comprises or consists of the amino acid sequence of SEQ ID NO: 283.

In another embodiment, the ISVD specifically binding to human PD-1 may have a sequence identity of more than 90%, such as more than 95% or even more than 99%, with SEQ ID NO: 284, wherein the CDRs are as defined above. In one embodiment, the ISVD specifically binding to PD-1 comprises or consists of the amino acid sequence of SEQ ID NO: 284.

In another embodiment, the ISVD specifically binding to human PD-1 may have a sequence identity of more than 90%, such as more than 95% or even more than 99%, with SEQ ID NO: 285, wherein the CDRs are as defined above. In one embodiment, the ISVD specifically binding to PD-1 comprises or consists of the amino acid sequence of SEQ ID NO: 285.

In another embodiment, the ISVD specifically binding to human PD-1 may have a sequence identity of more than 90%, such as more than 95% or even more than 99%, with SEQ ID NO: 286, wherein the CDRs are as defined above. In one embodiment, the ISVD specifically binding to PD-1 comprises or consists of the amino acid sequence of SEQ ID NO: 286.

In another embodiment, the ISVD specifically binding to human PD-1 may have a sequence identity of more than 90%, such as more than 95% or even more than 99%, with SEQ ID NO: 287, wherein the CDRs are as defined above. In one embodiment, the ISVD specifically binding to PD-1 comprises or consists of the amino acid sequence of SEQ ID NO: 287.

In another embodiment, the ISVD specifically binding to human PD-1 may have a sequence identity of more than 90%, such as more than 95% or even more than 99%, with SEQ ID NO: 288, wherein the CDRs are as defined above. In one embodiment, the ISVD specifically binding to PD-1 comprises or consists of the amino acid sequence of SEQ ID NO: 288.

In another embodiment, the ISVD specifically binding to human PD-1 may have a sequence identity of more than 90%, such as more than 95% or even more than 99%, with SEQ ID NO: 289, wherein the CDRs are as defined above. In one embodiment, the ISVD specifically binding to PD-1 comprises or consists of the amino acid sequence of SEQ ID NO: 289.

In another embodiment, the ISVD specifically binding to human PD-1 may have a sequence identity of more than 90%, such as more than 95% or even more than 99%, with SEQ ID NO: 290, wherein the CDRs are as defined above. In one embodiment, the ISVD specifically binding to PD-1 comprises or consists of the amino acid sequence of SEQ ID NO: 290.

In another embodiment, the ISVD specifically binding to human PD-1 may have a sequence identity of more than 90%, such as more than 95% or even more than 99%, with SEQ ID NO: 291, wherein the CDRs are as defined above. In one embodiment, the ISVD specifically binding to PD-1 comprises or consists of the amino acid sequence of SEQ ID NO: 291.

In another embodiment, the ISVD specifically binding to human PD-1 may have a sequence identity of more than 90%, such as more than 95% or even more than 99%, with SEQ ID NO: 292, wherein the CDRs are as defined above. In one embodiment, the ISVD specifically binding to PD-1 comprises or consists of the amino acid sequence of SEQ ID NO: 292.

In another embodiment, the ISVD specifically binding to human PD-1 may have a sequence identity of more than 90%, such as more than 95% or even more than 99%, with SEQ ID NO: 293, wherein the CDRs are as defined above. In one embodiment, the ISVD specifically binding to PD-1 comprises or consists of the amino acid sequence of SEQ ID NO: 293.

In another embodiment, the ISVD specifically binding to human PD-1 may have a sequence identity of more than 90%, such as more than 95% or even more than 99%, with SEQ ID NO: 294, wherein the CDRs are as defined above. In one embodiment, the ISVD specifically binding to PD-1 comprises or consists of the amino acid sequence of SEQ ID NO: 294.

In another embodiment, the ISVD specifically binding to human PD-1 may have a sequence identity of more than 90%, such as more than 95% or even more than 99%, with SEQ ID NO: 295, wherein the CDRs are as defined above. In one embodiment, the ISVD specifically binding to PD-1 comprises or consists of the amino acid sequence of SEQ ID NO: 295.

In another embodiment, the ISVD specifically binding to human PD-1 may have a sequence identity of more than 90%, such as more than 95% or even more than 99%, with SEQ ID NO: 296, wherein the CDRs are as defined above. In one embodiment, the ISVD specifically binding to PD-1 comprises or consists of the amino acid sequence of SEQ ID NO: 296.

In another embodiment, the ISVD specifically binding to human PD-1 may have a sequence identity of more than 90%, such as more than 95% or even more than 99%, with SEQ ID NO: 297, wherein the CDRs are as defined above. In one embodiment, the ISVD specifically binding to PD-1 comprises or consists of the amino acid sequence of SEQ ID NO: 297.

In another embodiment, the ISVD specifically binding to human PD-1 may have a sequence identity of more than 90%, such as more than 95% or even more than 99%, with SEQ ID NO: 298, wherein the CDRs are as defined above. In one embodiment, the ISVD specifically binding to PD-1 comprises or consists of the amino acid sequence of SEQ ID NO: 298.

In another embodiment, the ISVD specifically binding to human PD-1 may have a sequence identity of more than 90%, such as more than 95% or even more than 99%, with SEQ ID NO: 299, wherein the CDRs are as defined above. In one embodiment, the ISVD specifically binding to PD-1 comprises or consists of the amino acid sequence of SEQ ID NO: 299.

In another embodiment, the ISVD specifically binding to human PD-1 may have a sequence identity of more than 90%, such as more than 95% or even more than 99%, with SEQ ID NO: 300, wherein the CDRs are as defined above. In one embodiment, the ISVD specifically binding to PD-1 comprises or consists of the amino acid sequence of SEQ ID NO: 300.

In another embodiment, the ISVD specifically binding to human PD-1 may have a sequence identity of more than 90%, such as more than 95% or even more than 99%, with SEQ ID NO: 301, wherein the CDRs are as defined above. In one embodiment, the ISVD specifically binding to PD-1 comprises or consists of the amino acid sequence of SEQ ID NO: 301.

In another embodiment, the ISVD specifically binding to human PD-1 may have a sequence identity of more than 90%, such as more than 95% or even more than 99%, with SEQ ID NO: 302, wherein the CDRs are as defined above. In one embodiment, the ISVD specifically binding to PD-1 comprises or consists of the amino acid sequence of SEQ ID NO: 302.

In another embodiment, the ISVD specifically binding to human PD-1 may have a sequence Identity of more than 90%, such as more than 95% or even more than 99%, with SEQ ID NO: 303, wherein the CDRs are as defined above. In one embodiment, the ISVD specifically binding to PD-1 comprises or consists of the amino acid sequence of SEQ ID NO: 303.

In another embodiment, the ISVD specifically binding to human PD-1 may have a sequence identity of more than 90%, such as more than 95% or even more than 99%, with SEQ ID NO: 304, wherein the CDRs are as defined above. In one embodiment, the ISVD specifically binding to PD-1 comprises or consists of the amino acid sequence of SEQ ID NO: 304.

In another embodiment, the ISVD specifically binding to human PD-1 may have a sequence identity of more than 90%, such as more than 95% or even more than 99%, with SEQ ID NO: 305, wherein the CDRs are as defined above. In one embodiment, the ISVD specifically binding to PD-1 comprises or consists of the amino acid sequence of SEQ ID NO: 305.

In another embodiment, the ISVD specifically binding to human PD-1 may have a sequence identity of more than 90%, such as more than 95% or even more than 99%, with SEQ ID NO: 306, wherein the CDRs are as defined above. In one embodiment, the ISVD specifically binding to PD-1 comprises or consists of the amino acid sequence of SEQ ID NO: 306.

In another embodiment, the ISVD specifically binding to human PD-1 may have a sequence identity of more than 90%, such as more than 95% or even more than 99%, with SEQ ID NO: 307, wherein the CDRs are as defined above. In one embodiment, the ISVD specifically binding to PD-1 comprises or consists of the amino acid sequence of SEQ ID NO: 307.

In another embodiment, the ISVD specifically binding to human PD-1 may have a sequence identity of more than 90%, such as more than 95% or even more than 99%, with SEQ ID NO: 308, wherein the CDRs are as defined above. In one embodiment, the ISVD specifically binding to PD-1 comprises or consists of the amino acid sequence of SEQ ID NO: 308.

In another embodiment, the ISVD specifically binding to human PD-1 may have a sequence identity of more than 90%, such as more than 95% or even more than 99%, with SEQ ID NO: 309, wherein the CDRs are as defined above. In one embodiment, the ISVD specifically binding to PD-1 comprises or consists of the amino acid sequence of SEQ ID NO: 309.

In another embodiment, the ISVD specifically binding to human PD-1 may have a sequence identity of more than 90%, such as more than 95% or even more than 99%, with SEQ ID NO: 310, wherein the CDRs are as defined above. In one embodiment, the ISVD specifically binding to PD-1 comprises or consists of the amino acid sequence of SEQ ID NO: 310.

In another embodiment, the ISVD specifically binding to human PD-1 may have a sequence identity of more than 90%, such as more than 95% or even more than 99%, with SEQ ID NO: 311, wherein the CDRs are as defined above. In one embodiment, the ISVD specifically binding to PD-1 comprises or consists of the amino acid sequence of SEQ ID NO: 311.

In another embodiment, the ISVD specifically binding to human PD-1 may have a sequence identity of more than 90%, such as more than 95% or even more than 99%, with SEQ ID NO: 312, wherein the CDRs are as defined above. In one embodiment, the ISVD specifically binding to PD-1 comprises or consists of the amino acid sequence of SEQ ID NO: 312.

In another embodiment, the ISVD specifically binding to human PD-1 may have a sequence identity of more than 90%, such as more than 95% or even more than 99%, with SEQ ID NO: 313, wherein the CDRs are as defined above. In one embodiment, the ISVD specifically binding to PD-1 comprises or consists of the amino acid sequence of SEQ ID NO: 313.

In another embodiment, the ISVD specifically binding to human PD-1 may have a sequence identity of more than 90%, such as more than 95% or even more than 99%, with SEQ ID NO: 314, wherein the CDRs are as defined above. In one embodiment, the ISVD specifically binding to PD-1 comprises or consists of the amino acid sequence of SEQ ID NO: 314.

In another embodiment, the ISVD specifically binding to human PD-1 may have a sequence identity of more than 90%, such as more than 95% or even more than 99%, with SEQ ID NO: 315, wherein the CDRs are as defined above. In one embodiment, the ISVD specifically binding to PD-1 comprises or consists of the amino acid sequence of SEQ ID NO: 315.

In another embodiment, the ISVD specifically binding to human PD-1 may have a sequence identity of more than 90%, such as more than 95% or even more than 99%, with SEQ ID NO: 316, wherein the CDRs are as defined above. In one embodiment, the ISVD specifically binding to PD-1 comprises or consists of the amino acid sequence of SEQ ID NO: 316.

In another embodiment, the ISVD specifically binding to human PD-1 may have a sequence identity of more than 90%, such as more than 95% or even more than 99%, with SEQ ID NO: 317, wherein the CDRs are as defined above. In one embodiment, the ISVD specifically binding to PD-1 comprises or consists of the amino acid sequence of SEQ ID NO: 317.

In another embodiment, the ISVD specifically binding to human PD-1 may have a sequence identity of more than 90%, such as more than 95% or even more than 99%, with SEQ ID NO: 318, wherein the CDRs are as defined above. In one embodiment, the ISVD specifically binding to PD-1 comprises or consists of the amino acid sequence of SEQ ID NO: 318.

In another embodiment, the ISVD specifically binding to human PD-1 may have a sequence identity of more than 90%, such as more than 95% or even more than 99%, with SEQ ID NO: 319, wherein the CDRs are as defined above. In one embodiment, the ISVD specifically binding to PD-1 comprises or consists of the amino acid sequence of SEQ ID NO: 319.

In another embodiment, the ISVD specifically binding to human PD-1 may have a sequence identity of more than 90%, such as more than 95% or even more than 99%, with SEQ ID NO: 320, wherein the CDRs are as defined above. In one embodiment, the ISVD specifically binding to PD-1 comprises or consists of the amino acid sequence of SEQ ID NO: 320.

In another embodiment, the ISVD specifically binding to human PD-1 may have a sequence identity of more than 90%, such as more than 95% or even more than 99%, with SEQ ID NO: 321, wherein the CDRs are as defined above. In one embodiment, the ISVD specifically binding to PD-1 comprises or consists of the amino acid sequence of SEQ ID NO: 321.

In another embodiment, the ISVD specifically binding to human PD-1 may have a sequence identity of more than 90%, such as more than 95% or even more than 99%, with SEQ ID NO: 322, wherein the CDRs are as defined above. In one embodiment, the ISVD specifically binding to PD-1 comprises or consists of the amino acid sequence of SEQ ID NO: 322.

In another embodiment, the ISVD specifically binding to human PD-1 may have a sequence identity of more than 90%, such as more than 95% or even more than 99%, with SEQ ID NO: 323, wherein the CDRs are as defined above. In one embodiment, the ISVD specifically binding to PD-1 comprises or consists of the amino acid sequence of SEQ ID NO: 323.

In another embodiment, the ISVD specifically binding to human PD-1 may have a sequence identity of more than 90%, such as more than 95% or even more than 99%, with SEQ ID NO: 324, wherein the CDRs are as defined above. In one embodiment, the ISVD specifically binding to PD-1 comprises or consists of the amino acid sequence of SEQ ID NO: 324.

In another embodiment, the ISVD specifically binding to human PD-1 may have a sequence identity of more than 90%, such as more than 95% or even more than 99%, with SEQ ID NO: 325, wherein the CDRs are as defined above. In one embodiment, the ISVD specifically binding to PD-1 comprises or consists of the amino acid sequence of SEQ ID NO: 325.

In another embodiment, the ISVD specifically binding to human PD-1 may have a sequence identity of more than 90%, such as more than 95% or even more than 99%, with SEQ ID NO: 326, wherein the CDRs are as defined above. In one embodiment, the ISVD specifically binding to PD-1 comprises or consists of the amino acid sequence of SEQ ID NO: 326.

In another embodiment, the ISVD specifically binding to human PD-1 may have a sequence identity of more than 90%, such as more than 95% or even more than 99%, with SEQ ID NO: 327, wherein the CDRs are as defined above. In one embodiment, the ISVD specifically binding to PD-1 comprises or consists of the amino acid sequence of SEQ ID NO: 327.

In another embodiment, the ISVD specifically binding to human PD-1 may have a sequence identity of more than 90%, such as more than 95% or even more than 99%, with SEQ ID NO: 328, wherein the CDRs are as defined above. In one embodiment, the ISVD specifically binding to PD-1 comprises or consists of the amino acid sequence of SEQ ID NO: 328.

In another embodiment, the ISVD specifically binding to human PD-1 may have a sequence identity of more than 90%, such as more than 95% or even more than 99%, with SEQ ID NO: 329, wherein the CDRs are as defined above. In one embodiment, the ISVD specifically binding to PD-1 comprises or consists of the amino acid sequence of SEQ ID NO: 329.

In another embodiment, the ISVD specifically binding to human PD-1 may have a sequence identity of more than 90%, such as more than 95% or even more than 99%, with SEQ ID NO: 330, wherein the CDRs are as defined above. In one embodiment, the ISVD specifically binding to PD-1 comprises or consists of the amino acid sequence of SEQ ID NO: 330.

In another embodiment, the ISVD specifically binding to human PD-1 may have a sequence identity of more than 90%, such as more than 95% or even more than 99%, with SEQ ID NO: 331, wherein the CDRs are as defined above. In one embodiment, the ISVD specifically binding to PD-1 comprises or consists of the amino acid sequence of SEQ ID NO: 331.

In another embodiment, the ISVD specifically binding to human PD-1 may have a sequence identity of more than 90%, such as more than 95% or even more than 99%, with SEQ ID NO: 332, wherein the CDRs are as defined above. In one embodiment, the ISVD specifically binding to PD-1 comprises or consists of the amino acid sequence of SEQ ID NO: 332.

In another embodiment, the ISVD specifically binding to human PD-1 may have a sequence identity of more than 90%, such as more than 95% or even more than 99%, with SEQ ID NO: 333, wherein the CDRs are as defined above. In one embodiment, the ISVD specifically binding to PD-1 comprises or consists of the amino acid sequence of SEQ ID NO: 333.

In another embodiment, the ISVD specifically binding to human PD-1 may have a sequence identity of more than 90%, such as more than 95% or even more than 99%, with SEQ ID NO: 334, wherein the CDRs are as defined above. In one embodiment, the ISVD specifically binding to PD-1 comprises or consists of the amino acid sequence of SEQ ID NO: 334.

In another embodiment, the ISVD specifically binding to human PD-1 may have a sequence identity of more than 90%, such as more than 95% or even more than 99%, with SEQ ID NO: 335, wherein the CDRs are as defined above. In one embodiment, the ISVD specifically binding to PD-1 comprises or consists of the amino acid sequence of SEQ ID NO: 335.

In another embodiment, the ISVD specifically binding to human PD-1 may have a sequence identity of more than 90%, such as more than 95% or even more than 99%, with SEQ ID NO: 336, wherein the CDRs are as defined above. In one embodiment, the ISVD specifically binding to PD-1 comprises or consists of the amino acid sequence of SEQ ID NO: 336.

In another embodiment, the ISVD specifically binding to human PD-1 may have a sequence identity of more than 90%, such as more than 95% or even more than 99%, with SEQ ID NO: 337, wherein the CDRs are as defined above. In one embodiment, the ISVD specifically binding to PD-1 comprises or consists of the amino acid sequence of SEQ ID NO: 337.

In another embodiment, the ISVD specifically binding to human PD-1 may have a sequence identity of more than 90%, such as more than 95% or even more than 99%, with SEQ ID NO: 338, wherein the CDRs are as defined above. In one embodiment, the ISVD specifically binding to PD-1 comprises or consists of the amino acid sequence of SEQ ID NO: 338.

In another embodiment, the ISVD specifically binding to human PD-1 may have a sequence identity of more than 90%, such as more than 95% or even more than 99%, with SEQ ID NO: 339, wherein the CDRs are as defined above. In one embodiment, the ISVD specifically binding to PD-1 comprises or consists of the amino acid sequence of SEQ ID NO: 339.

In another embodiment, the ISVD specifically binding to human PD-1 may have a sequence identity of more than 90%, such as more than 95% or even more than 99%, with SEQ ID NO: 340, wherein the CDRs are as defined above. In one embodiment, the ISVD specifically binding to PD-1 comprises or consists of the amino acid sequence of SEQ ID NO: 340.

In another embodiment, the ISVD specifically binding to human PD-1 may have a sequence identity of more than 90%, such as more than 95% or even more than 99%, with SEQ ID NO: 341, wherein the CDRs are as defined above. In one embodiment, the ISVD specifically binding to PD-1 comprises or consists of the amino acid sequence of SEQ ID NO: 341.

In another embodiment, the ISVD specifically binding to human PD-1 may have a sequence identity of more than 90%, such as more than 95% or even more than 99%, with SEQ ID NO: 342, wherein the CDRs are as defined above. In one embodiment, the ISVD specifically binding to PD-1 comprises or consists of the amino acid sequence of SEQ ID NO: 342.

In another embodiment, the ISVD specifically binding to human PD-1 may have a sequence identity of more than 90%, such as more than 95% or even more than 99%, with SEQ ID NO: 343, wherein the CDRs are as defined above. In one embodiment, the ISVD specifically binding to PD-1 comprises or consists of the amino acid sequence of SEQ ID NO: 343.

In another embodiment, the ISVD specifically binding to human PD-1 may have a sequence identity of more than 90%, such as more than 95% or even more than 99%, with SEQ ID NO: 344, wherein the CDRs are as defined above. In one embodiment, the ISVD specifically binding to PD-1 comprises or consists of the amino acid sequence of SEQ ID NO: 344.

In another embodiment, the ISVD specifically binding to human PD-1 may have a sequence identity of more than 90%, such as more than 95% or even more than 99%, with SEQ ID NO: 345, wherein the CDRs are as defined above. In one embodiment, the ISVD specifically binding to PD-1 comprises or consists of the amino acid sequence of SEQ ID NO: 345.

In another embodiment, the ISVD specifically binding to human PD-1 may have a sequence identity of more than 90%, such as more than 95% or even more than 99%, with SEQ ID NO: 346, wherein the CDRs are as defined above. In one embodiment, the ISVD specifically binding to PD-1 comprises or consists of the amino acid sequence of SEQ ID NO: 346.

In another embodiment, the ISVD specifically binding to human PD-1 may have a sequence identity of more than 90%, such as more than 95% or even more than 99%, with SEQ ID NO: 347, wherein the CDRs are as defined above. In one embodiment, the ISVD specifically binding to PD-1 comprises or consists of the amino acid sequence of SEQ ID NO: 347.

In another embodiment, the ISVD specifically binding to human PD-1 may have a sequence identity of more than 90%, such as more than 95% or even more than 99%, with SEQ ID NO: 348, wherein the CDRs are as defined above. In one embodiment, the ISVD specifically binding to PD-1 comprises or consists of the amino acid sequence of SEQ ID NO: 348.

In another embodiment, the ISVD specifically binding to human PD-1 may have a sequence identity of more than 90%, such as more than 95% or even more than 99%, with SEQ ID NO: 349, wherein the CDRs are as defined above. In one embodiment, the ISVD specifically binding to PD-1 comprises or consists of the amino acid sequence of SEQ ID NO: 349.

In another embodiment, the ISVD specifically binding to human PD-1 may have a sequence identity of more than 90%, such as more than 95% or even more than 99%, with SEQ ID NO: 350, wherein the CDRs are as defined above. In one embodiment, the ISVD specifically binding to PD-1 comprises or consists of the amino acid sequence of SEQ ID NO: 350.

In another embodiment, the ISVD specifically binding to human PD-1 may have a sequence identity of more than 90%, such as more than 95% or even more than 99%, with SEQ ID NO: 351, wherein the CDRs are as defined above. In one embodiment, the ISVD specifically binding to PD-1 comprises or consists of the amino acid sequence of SEQ ID NO: 351.

In another embodiment, the ISVD specifically binding to human PD-1 may have a sequence identity of more than 90%, such as more than 95% or even more than 99%, with SEQ ID NO: 352, wherein the CDRs are as defined above. In one embodiment, the ISVD specifically binding to PD-1 comprises or consists of the amino acid sequence of SEQ ID NO: 352.

In another embodiment, the ISVD specifically binding to human PD-1 may have a sequence identity of more than 90%, such as more than 95% or even more than 99%, with SEQ ID NO: 353, wherein the CDRs are as defined above. In one embodiment, the ISVD specifically binding to PD-1 comprises or consists of the amino acid sequence of SEQ ID NO: 353.

In another embodiment, the ISVD specifically binding to human PD-1 may have a sequence identity of more than 90%, such as more than 95% or even more than 99%, with SEQ ID NO: 354, wherein the CDRs are as defined above. In one embodiment, the ISVD specifically binding to PD-1 comprises or consists of the amino acid sequence of SEQ ID NO: 354.

In another embodiment, the ISVD specifically binding to human PD-1 may have a sequence identity of more than 90%, such as more than 95% or even more than 99%, with SEQ ID NO: 355, wherein the CDRs are as defined above. In one embodiment, the ISVD specifically binding to PD-1 comprises or consists of the amino acid sequence of SEQ ID NO: 355.

In another embodiment, the ISVD specifically binding to human PD-1 may have a sequence identity of more than 90%, such as more than 95% or even more than 99%, with SEQ ID NO: 356, wherein the CDRs are as defined above. In one embodiment, the ISVD specifically binding to PD-1 comprises or consists of the amino acid sequence of SEQ ID NO: 356.

In another embodiment, the ISVD specifically binding to human PD-1 may have a sequence identity of more than 90%, such as more than 95% or even more than 99%, with SEQ ID NO: 357, wherein the CDRs are as defined above. In one embodiment, the ISVD specifically binding to PD-1 comprises or consists of the amino acid sequence of SEQ ID NO: 357.

In another embodiment, the ISVD specifically binding to human PD-1 may have a sequence identity of more than 90%, such as more than 95% or even more than 99%, with SEQ ID NO: 358, wherein the CDRs are as defined above. In one embodiment, the ISVD specifically binding to PD-1 comprises or consists of the amino acid sequence of SEQ ID NO: 358.

In another embodiment, the ISVD specifically binding to human PD-1 may have a sequence identity of more than 90%, such as more than 95% or even more than 99%, with SEQ ID NO: 359, wherein the CDRs are as defined above. In one embodiment, the ISVD specifically binding to PD-1 comprises or consists of the amino acid sequence of SEQ ID NO: 359.

In another embodiment, the ISVD specifically binding to human PD-1 may have a sequence identity of more than 90%, such as more than 95% or even more than 99%, with SEQ ID NO: 360, wherein the CDRs are as defined above. In one embodiment, the ISVD specifically binding to PD-1 comprises or consists of the amino acid sequence of SEQ ID NO: 360.

In another embodiment, the ISVD specifically binding to human PD-1 may have a sequence identity of more than 90%, such as more than 95% or even more than 99%, with SEQ ID NO: 361, wherein the CDRs are as defined above. In one embodiment, the ISVD specifically binding to PD-1 comprises or consists of the amino acid sequence of SEQ ID NO: 361.

In another embodiment, the ISVD specifically binding to human PD-1 may have a sequence identity of more than 90%, such as more than 95% or even more than 99%, with SEQ ID NO: 362, wherein the CDRs are as defined above. In one embodiment, the ISVD specifically binding to PD-1 comprises or consists of the amino acid sequence of SEQ ID NO: 362.

In another embodiment, the ISVD specifically binding to human PD-1 may have a sequence identity of more than 90%, such as more than 95% or even more than 99%, with SEQ ID NO: 363, wherein the CDRs are as defined above. In one embodiment, the ISVD specifically binding to PD-1 comprises or consists of the amino acid sequence of SEQ ID NO: 363.

In another embodiment, the ISVD specifically binding to human PD-1 may have a sequence identity of more than 90%, such as more than 95% or even more than 99%, with SEQ ID NO: 364, wherein the CDRs are as defined above. In one embodiment, the ISVD specifically binding to PD-1 comprises or consists of the amino acid sequence of SEQ ID NO: 364.

In another embodiment, the ISVD specifically binding to human PD-1 may have a sequence identity of more than 90%, such as more than 95% or even more than 99%, with SEQ ID NO: 365, wherein the CDRs are as defined above. In one embodiment, the ISVD specifically binding to PD-1 comprises or consists of the amino acid sequence of SEQ ID NO: 365.

In another embodiment, the ISVD specifically binding to human PD-1 may have a sequence identity of more than 90%, such as more than 95% or even more than 99%, with SEQ ID NO: 366, wherein the CDRs are as defined above. In one embodiment, the ISVD specifically binding to PD-1 comprises or consists of the amino acid sequence of SEQ ID NO: 366.

In another embodiment, the ISVD specifically binding to human PD-1 may have a sequence identity of more than 90%, such as more than 95% or even more than 99%, with SEQ ID NO: 367, wherein the CDRs are as defined above. In one embodiment, the ISVD specifically binding to PD-1 comprises or consists of the amino acid sequence of SEQ ID NO: 367.

In another embodiment, the ISVD specifically binding to human PD-1 may have a sequence identity of more than 90%, such as more than 95% or even more than 99%, with SEQ ID NO: 368, wherein the CDRs are as defined above. In one embodiment, the ISVD specifically binding to PD-1 comprises or consists of the amino acid sequence of SEQ ID NO: 368.

In another embodiment, the ISVD specifically binding to human PD-1 may have a sequence identity of more than 90%, such as more than 95% or even more than 99%, with SEQ ID NO: 369, wherein the CDRs are as defined above. In one embodiment, the ISVD specifically binding to PD-1 comprises or consists of the amino acid sequence of SEQ ID NO: 369.

In another embodiment, the ISVD specifically binding to human PD-1 may have a sequence identity of more than 90%, such as more than 95% or even more than 99%, with SEQ ID NO: 370, wherein the CDRs are as defined above. In one embodiment, the ISVD specifically binding to PD-1 comprises or consists of the amino acid sequence of SEQ ID NO: 370.

In another embodiment, the ISVD specifically binding to human PD-1 may have a sequence identity of more than 90%, such as more than 95% or even more than 99%, with SEQ ID NO: 371, wherein the CDRs are as defined above. In one embodiment, the ISVD specifically binding to PD-1 comprises or consists of the amino acid sequence of SEQ ID NO: 371.

In another embodiment, the ISVD specifically binding to human PD-1 may have a sequence identity of more than 90%, such as more than 95% or even more than 99%, with SEQ ID NO: 372, wherein the CDRs are as defined above. In one embodiment, the ISVD specifically binding to PD-1 comprises or consists of the amino acid sequence of SEQ ID NO: 372.

In another embodiment, the ISVD specifically binding to human PD-1 may have a sequence identity of more than 90%, such as more than 95% or even more than 99%, with SEQ ID NO: 373, wherein the CDRs are as defined above. In one embodiment, the ISVD specifically binding to PD-1 comprises or consists of the amino acid sequence of SEQ ID NO: 373.

In another embodiment, the ISVD specifically binding to human PD-1 may have a sequence identity of more than 90%, such as more than 95% or even more than 99%, with SEQ ID NO: 374, wherein the CDRs are as defined above. In one embodiment, the ISVD specifically binding to PD-1 comprises or consists of the amino acid sequence of SEQ ID NO: 374.

In another embodiment, the ISVD specifically binding to human PD-1 may have a sequence identity of more than 90%, such as more than 95% or even more than 99%, with SEQ ID NO: 375, wherein the CDRs are as defined above. In one embodiment, the ISVD specifically binding to PD-1 comprises or consists of the amino acid sequence of SEQ ID NO: 375.

In another embodiment, the ISVD specifically binding to human PD-1 may have a sequence identity of more than 90%, such as more than 95% or even more than 99%, with SEQ ID NO: 376, wherein the CDRs are as defined above. In one embodiment, the ISVD specifically binding to PD-1 comprises or consists of the amino acid sequence of SEQ ID NO: 376.

In another embodiment, the ISVD specifically binding to human PD-1 may have a sequence identity of more than 90%, such as more than 95% or even more than 99%, with SEQ ID NO: 377, wherein the CDRs are as defined above. In one embodiment, the ISVD specifically binding to PD-1 comprises or consists of the amino acid sequence of SEQ ID NO: 377.

In another embodiment, the ISVD specifically binding to human PD-1 may have a sequence identity of more than 90%, such as more than 95% or even more than 99%, with SEQ ID NO: 378, wherein the CDRs are as defined above. In one embodiment, the ISVD specifically binding to PD-1 comprises or consists of the amino acid sequence of SEQ ID NO: 378.

In another embodiment, the ISVD specifically binding to human PD-1 may have a sequence identity of more than 90%, such as more than 95% or even more than 99%, with SEQ ID NO: 379, wherein the CDRs are as defined above. In one embodiment, the ISVD specifically binding to PD-1 comprises or consists of the amino acid sequence of SEQ ID NO: 379.

In another embodiment, the ISVD specifically binding to human PD-1 may have a sequence identity of more than 90%, such as more than 95% or even more than 99%, with SEQ ID NO: 380, wherein the CDRs are as defined above. In one embodiment, the ISVD specifically binding to PD-1 comprises or consists of the amino acid sequence of SEQ ID NO: 380.

In another embodiment, the ISVD specifically binding to human PD-1 may have a sequence identity of more than 90%, such as more than 95% or even more than 99%, with SEQ ID NO: 381, wherein the CDRs are as defined above. In one embodiment, the ISVD specifically binding to PD-1 comprises or consists of the amino acid sequence of SEQ ID NO: 381.

In another embodiment, the ISVD specifically binding to human PD-1 may have a sequence identity of more than 90%, such as more than 95% or even more than 99%, with SEQ ID NO: 382, wherein the CDRs are as defined above. In one embodiment, the ISVD specifically binding to PD-1 comprises or consists of the amino acid sequence of SEQ ID NO: 382.

In another embodiment, the ISVD specifically binding to human PD-1 may have a sequence identity of more than 90%, such as more than 95% or even more than 99%, with SEQ ID NO: 383, wherein the CDRs are as defined above. In one embodiment, the ISVD specifically binding to PD-1 comprises or consists of the amino acid sequence of SEQ ID NO: 383.

In another embodiment, the ISVD specifically binding to human PD-1 may have a sequence identity of more than 90%, such as more than 95% or even more than 99%, with SEQ ID NO: 384, wherein the CDRs are as defined above. In one embodiment, the ISVD specifically binding to PD-1 comprises or consists of the amino acid sequence of SEQ ID NO: 384.

In another embodiment, the ISVD specifically binding to human PD-1 may have a sequence identity of more than 90%, such as more than 95% or even more than 99%, with SEQ ID NO: 385, wherein the CDRs are as defined above. In one embodiment, the ISVD specifically binding to PD-1 comprises or consists of the amino acid sequence of SEQ ID NO: 385.

In another embodiment, the ISVD specifically binding to human PD-1 may have a sequence identity of more than 90%, such as more than 95% or even more than 99%, with SEQ ID NO: 386, wherein the CDRs are as defined above. In one embodiment, the ISVD specifically binding to PD-1 comprises or consists of the amino acid sequence of SEQ ID NO: 386.

In another embodiment, the ISVD specifically binding to human PD-1 may have a sequence identity of more than 90%, such as more than 95% or even more than 99%, with SEQ ID NO: 387, wherein the CDRs are as defined above. In one embodiment, the ISVD specifically binding to PD-1 comprises or consists of the amino acid sequence of SEQ ID NO: 387.

In another embodiment, the ISVD specifically binding to human PD-1 may have a sequence identity of more than 90%, such as more than 95% or even more than 99%, with SEQ ID NO: 388, wherein the CDRs are as defined above. In one embodiment, the ISVD specifically binding to PD-1 comprises or consists of the amino acid sequence of SEQ ID NO: 388.

30. In another embodiment, the ISVD specifically binding to human PD-1 may have a sequence identity of more than 90%, such as more than 95% or even more than 99%, with SEQ ID NO: 389, wherein the CDRs are as defined above. In one embodiment, the ISVD specifically binding to PD-1 comprises or consists of the amino acid sequence of SEQ ID NO: 389.

In another embodiment, the ISVD specifically binding to human PD-1 may have a sequence identity of more than 90%, such as more than 95% or even more than 99%, with SEQ ID NO: 390, wherein the CDRs are as defined above. In one embodiment, the ISVD specifically binding to PD-1 comprises or consists of the amino acid sequence of SEQ ID NO: 390.

In another embodiment, the ISVD specifically binding to human PD-1 may have a sequence identity of more than 90%, such as more than 95% or even more than 99%, with SEQ ID NO: 391, wherein the CDRs are as defined above. In one embodiment, the ISVD specifically binding to PD-1 comprises or consists of the amino acid sequence of SEQ ID NO: 391.

In another embodiment, the ISVD specifically binding to human PD-1 may have a sequence identity of more than 90%, such as more than 95% or even more than 99%, with SEQ ID NO: 392, wherein the CDRs are as defined above. In one embodiment, the ISVD specifically binding to PD-1 comprises or consists of the amino acid sequence of SEQ ID NO: 392.

In another embodiment, the ISVD specifically binding to human PD-1 may have a sequence identity of more than 90%, such as more than 95% or even more than 99%, with SEQ ID NO: 393, wherein the CDRs are as defined above. In one embodiment, the ISVD specifically binding to PD-1 comprises or consists of the amino acid sequence of SEQ ID NO: 393.

In another embodiment, the ISVD specifically binding to human PD-1 may have a sequence identity of more than 90%, such as more than 95% or even more than 99%, with SEQ ID NO: 394, wherein the CDRs are as defined above. In one embodiment, the ISVD specifically binding to PD-1 comprises or consists of the amino acid sequence of SEQ ID NO: 394.

In another embodiment, the ISVD specifically binding to human PD-1 may have a sequence identity of more than 90%, such as more than 95% or even more than 99%, with SEQ ID NO: 395, wherein the CDRs are as defined above. In one embodiment, the ISVD specifically binding to PD-1 comprises or consists of the amino acid sequence of SEQ ID NO: 395.

In another embodiment, the ISVD specifically binding to human PD-1 may have a sequence identity of more than 90%, such as more than 95% or even more than 99%, with SEQ ID NO: 396, wherein the CDRs are as defined above. In one embodiment, the ISVD specifically binding to PD-1 comprises or consists of the amino acid sequence of SEQ ID NO: 396.

In another embodiment, the ISVD specifically binding to human PD-1 may have a sequence identity of more than 90%, such as more than 95% or even more than 99%, with SEQ ID NO: 397, wherein the CDRs are as defined above. In one embodiment, the ISVD specifically binding to PD-1 comprises or consists of the amino acid sequence of SEQ ID NO: 397.

In another embodiment, the ISVD specifically binding to human PD-1 may have a sequence identity of more than 90%, such as more than 95% or even more than 99%, with SEQ ID NO: 398, wherein the CDRs are as defined above. In one embodiment, the ISVD specifically binding to PD-1 comprises or consists of the amino acid sequence of SEQ ID NO: 398.

In another embodiment, the ISVD specifically binding to human PD-1 may have a sequence identity of more than 90%, such as more than 95% or even more than 99%, with SEQ ID NO: 399, wherein the CDRs are as defined above. In one embodiment, the ISVD specifically binding to PD-1 comprises or consists of the amino acid sequence of SEQ ID NO: 399.

In another embodiment, the ISVD specifically binding to human PD-1 may have a sequence identity of more than 90%, such as more than 95% or even more than 99%, with SEQ ID NO: 400, wherein the CDRs are as defined above. In one embodiment, the ISVD specifically binding to PD-1 comprises or consists of the amino acid sequence of SEQ ID NO: 400.

In another embodiment, the ISVD specifically binding to human PD-1 may have a sequence identity of more than 90%, such as more than 95% or even more than 99%, with SEQ ID NO: 401, wherein the CDRs are as defined above. In one embodiment, the ISVD specifically binding to PD-1 comprises or consists of the amino acid sequence of SEQ ID NO: 401.

In another embodiment, the ISVD specifically binding to human PD-1 may have a sequence identity of more than 90%, such as more than 95% or even more than 99%, with SEQ ID NO: 402, wherein the CDRs are as defined above. In one embodiment, the ISVD specifically binding to PD-1 comprises or consists of the amino acid sequence of SEQ ID NO: 402.

In another embodiment, the ISVD specifically binding to human PD-1 may have a sequence Identity of more than 90%, such as more than 95% or even more than 99%, with SEQ ID NO: 403, wherein the CDRs are as defined above. In one embodiment, the ISVD specifically binding to PD-1 comprises or consists of the amino acid sequence of SEQ ID NO: 403.

In another embodiment, the ISVD specifically binding to human PD-1 may have a sequence identity of more than 90%, such as more than 95% or even more than 99%, with SEQ ID NO: 404, wherein the CDRs are as defined above. In one embodiment, the ISVD specifically binding to PD-1 comprises or consists of the amino acid sequence of SEQ ID NO: 404.

In another embodiment, the ISVD specifically binding to human PD-1 may have a sequence identity of more than 90%, such as more than 95% or even more than 99%, with SEQ ID NO: 405, wherein the CDRs are as defined above. In one embodiment, the ISVD specifically binding to PD-1 comprises or consists of the amino acid sequence of SEQ ID NO: 405.

In another embodiment, the ISVD specifically binding to human PD-1 may have a sequence identity of more than 90%, such as more than 95% or even more than 99%, with SEQ ID NO: 406, wherein the CDRs are as defined above. In one embodiment, the ISVD specifically binding to PD-1 comprises or consists of the amino acid sequence of SEQ ID NO: 406.

In another embodiment, the ISVD specifically binding to human PD-1 may have a sequence identity of more than 90%, such as more than 95% or even more than 99%, with SEQ ID NO: 407, wherein the CDRs are as defined above. In one embodiment, the ISVD specifically binding to PD-1 comprises or consists of the amino acid sequence of SEQ ID NO: 407.

In another embodiment, the ISVD specifically binding to human PD-1 may have a sequence identity of more than 90%, such as more than 95% or even more than 99%, with SEQ ID NO: 408, wherein the CDRs are as defined above. In one embodiment, the ISVD specifically binding to PD-1 comprises or consists of the amino acid sequence of SEQ ID NO: 408.

In another embodiment, the ISVD specifically binding to human PD-1 may have a sequence identity of more than 90%, such as more than 95% or even more than 99%, with SEQ ID NO: 409, wherein the CDRs are as defined above. In one embodiment, the ISVD specifically binding to PD-1 comprises or consists of the amino acid sequence of SEQ ID NO: 409.

In another embodiment, the ISVD specifically binding to human PD-1 may have a sequence identity of more than 90%, such as more than 95% or even more than 99%, with SEQ ID NO: 410, wherein the CDRs are as defined above. In one embodiment, the ISVD specifically binding to PD-1 comprises or consists of the amino acid sequence of SEQ ID NO: 410.

In another embodiment, the ISVD specifically binding to human PD-1 may have a sequence identity of more than 90%, such as more than 95% or even more than 99%, with SEQ ID NO: 411, wherein the CDRs are as defined above. In one embodiment, the ISVD specifically binding to PD-1 comprises or consists of the amino acid sequence of SEQ ID NO: 411.

In another embodiment, the ISVD specifically binding to human PD-1 may have a sequence identity of more than 90%, such as more than 95% or even more than 99%, with SEQ ID NO: 412, wherein the CDRs are as defined above. In one embodiment, the ISVD specifically binding to PD-1 comprises or consists of the amino acid sequence of SEQ ID NO: 412.

In another embodiment, the ISVD specifically binding to human PD-1 may have a sequence identity of more than 90%, such as more than 95% or even more than 99%, with SEQ ID NO: 413, wherein the CDRs are as defined above. In one embodiment, the ISVD specifically binding to PD-1 comprises or consists of the amino acid sequence of SEQ ID NO: 413.

In another embodiment, the ISVD specifically binding to human PD-1 may have a sequence identity of more than 90%, such as more than 95% or even more than 99%, with SEQ ID NO: 414, wherein the CDRs are as defined above. In one embodiment, the ISVD specifically binding to PD-1 comprises or consists of the amino acid sequence of SEQ ID NO: 414.

In another embodiment, the ISVD specifically binding to human PD-1 may have a sequence identity of more than 90%, such as more than 95% or even more than 99%, with SEQ ID NO: 415, wherein the CDRs are as defined above. In one embodiment, the ISVD specifically binding to PD-1 comprises or consists of the amino acid sequence of SEQ ID NO: 415.

In another embodiment, the ISVD specifically binding to human PD-1 may have a sequence identity of more than 90%, such as more than 95% or even more than 99%, with SEQ ID NO: 416, wherein the CDRs are as defined above. In one embodiment, the ISVD specifically binding to PD-1 comprises or consists of the amino acid sequence of SEQ ID NO: 416.

In another embodiment, the ISVD specifically binding to human PD-1 may have a sequence identity of more than 90%, such as more than 95% or even more than 99%, with SEQ ID NO: 417, wherein the CDRs are as defined above. In one embodiment, the ISVD specifically binding to PD-1 comprises or consists of the amino acid sequence of SEQ ID NO: 417.

In another embodiment, the ISVD specifically binding to human PD-1 may have a sequence identity of more than 90%, such as more than 95% or even more than 99%, with SEQ ID NO: 418, wherein the CDRs are as defined above. In one embodiment, the ISVD specifically binding to PD-1 comprises or consists of the amino acid sequence of SEQ ID NO: 418.

In another embodiment, the ISVD specifically binding to human PD-1 may have a sequence identity of more than 90%, such as more than 95% or even more than 99%, with SEQ ID NO: 419, wherein the CDRs are as defined above. In one embodiment, the ISVD specifically binding to PD-1 comprises or consists of the amino acid sequence of SEQ ID NO: 419.

In another embodiment, the ISVD specifically binding to human PD-1 may have a sequence identity of more than 90%, such as more than 95% or even more than 99%, with SEQ ID NO: 420, wherein the CDRs are as defined above. In one embodiment, the ISVD specifically binding to PD-1 comprises or consists of the amino acid sequence of SEQ ID NO: 420.

In another embodiment, the ISVD specifically binding to human PD-1 may have a sequence identity of more than 90%, such as more than 95% or even more than 99%, with SEQ ID NO: 421, wherein the CDRs are as defined above. In one embodiment, the ISVD specifically binding to PD-1 comprises or consists of the amino acid sequence of SEQ ID NO: 421.

In another embodiment, the ISVD specifically binding to human PD-1 may have a sequence identity of more than 90%, such as more than 95% or even more than 99%, with SEQ ID NO: 422, wherein the CDRs are as defined above. In one embodiment, the ISVD specifically binding to PD-1 comprises or consists of the amino acid sequence of SEQ ID NO: 422.

In another embodiment, the ISVD specifically binding to human PD-1 may have a sequence identity of more than 90%, such as more than 95% or even more than 99%, with SEQ ID NO: 423, wherein the CDRs are as defined above. In one embodiment, the ISVD specifically binding to PD-1 comprises or consists of the amino acid sequence of SEQ ID NO: 423.

In another embodiment, the ISVD specifically binding to human PD-1 may have a sequence identity of more than 90%, such as more than 95% or even more than 99%, with SEQ ID NO: 424, wherein the CDRs are as defined above. In one embodiment, the ISVD specifically binding to PD-1 comprises or consists of the amino acid sequence of SEQ ID NO: 424.

In another embodiment, the ISVD specifically binding to human PD-1 may have a sequence identity of more than 90%, such as more than 95% or even more than 99%, with SEQ ID NO: 425, wherein the CDRs are as defined above. In one embodiment, the ISVD specifically binding to PD-1 comprises or consists of the amino acid sequence of SEQ ID NO: 425.

In another embodiment, the ISVD specifically binding to human PD-1 may have a sequence identity of more than 90%, such as more than 95% or even more than 99%, with SEQ ID NO: 426, wherein the CDRs are as defined above. In one embodiment, the ISVD specifically binding to PD-1 comprises or consists of the amino acid sequence of SEQ ID NO: 426.

In another embodiment, the ISVD specifically binding to human PD-1 may have a sequence identity of more than 90%, such as more than 95% or even more than 99%, with SEQ ID NO: 427, wherein the CDRs are as defined above. In one embodiment, the ISVD specifically binding to PD-1 comprises or consists of the amino acid sequence of SEQ ID NO: 427.

In another embodiment, the ISVD specifically binding to human PD-1 may have a sequence identity of more than 90%, such as more than 95% or even more than 99%, with SEQ ID NO: 428, wherein the CDRs are as defined above. In one embodiment, the ISVD specifically binding to PD-1 comprises or consists of the amino acid sequence of SEQ ID NO: 428.

In another embodiment, the ISVD specifically binding to human PD-1 may have a sequence identity of more than 90%, such as more than 95% or even more than 99%, with SEQ ID NO: 429, wherein the CDRs are as defined above. In one embodiment, the ISVD specifically binding to PD-1 comprises or consists of the amino acid sequence of SEQ ID NO: 429.

In another embodiment, the ISVD specifically binding to human PD-1 may have a sequence identity of more than 90%, such as more than 95% or even more than 99%, with SEQ ID NO: 430, wherein the CDRs are as defined above. In one embodiment, the ISVD specifically binding to PD-1 comprises or consists of the amino acid sequence of SEQ ID NO: 430.

In another embodiment, the ISVD specifically binding to human PD-1 may have a sequence identity of more than 90%, such as more than 95% or even more than 99%, with SEQ ID NO: 431, wherein the CDRs are as defined above. In one embodiment, the ISVD specifically binding to PD-1 comprises or consists of the amino acid sequence of SEQ ID NO: 431.

In another embodiment, the ISVD specifically binding to human PD-1 may have a sequence identity of more than 90%, such as more than 95% or even more than 99%, with SEQ ID NO: 432, wherein the CDRs are as defined above. In one embodiment, the ISVD specifically binding to PD-1 comprises or consists of the amino acid sequence of SEQ ID NO: 432.

In another embodiment, the ISVD specifically binding to human PD-1 may have a sequence identity of more than 90%, such as more than 95% or even more than 99%, with SEQ ID NO: 433, wherein the CDRs are as defined above. In one embodiment, the ISVD specifically binding to PD-1 comprises or consists of the amino acid sequence of SEQ ID NO: 433.

In another embodiment, the ISVD specifically binding to human PD-1 may have a sequence identity of more than 90%, such as more than 95% or even more than 99%, with SEQ ID NO: 434, wherein the CDRs are as defined above. In one embodiment, the ISVD specifically binding to PD-1 comprises or consists of the amino acid sequence of SEQ ID NO: 434.

In another embodiment, the ISVD specifically binding to human PD-1 may have a sequence identity of more than 90%, such as more than 95% or even more than 99%, with SEQ ID NO: 435, wherein the CDRs are as defined above. In one embodiment, the ISVD specifically binding to PD-1 comprises or consists of the amino acid sequence of SEQ ID NO: 435.

In another embodiment, the ISVD specifically binding to human PD-1 may have a sequence identity of more than 90%, such as more than 95% or even more than 99%, with SEQ ID NO: 436, wherein the CDRs are as defined above. In one embodiment, the ISVD specifically binding to PD-1 comprises or consists of the amino acid sequence of SEQ ID NO: 436.

In another embodiment, the ISVD specifically binding to human PD-1 may have a sequence identity of more than 90%, such as more than 95% or even more than 99%, with SEQ ID NO: 437, wherein the CDRs are as defined above. In one embodiment, the ISVD specifically binding to PD-1 comprises or consists of the amino acid sequence of SEQ ID NO: 437.

In another embodiment, the ISVD specifically binding to human PD-1 may have a sequence identity of more than 90%, such as more than 95% or even more than 99%, with SEQ ID NO: 438, wherein the CDRs are as defined above. In one embodiment, the ISVD specifically binding to PD-1 comprises or consists of the amino acid sequence of SEQ ID NO: 438.

In another embodiment, the ISVD specifically binding to human PD-1 may have a sequence identity of more than 90%, such as more than 95% or even more than 99%, with SEQ ID NO: 439, wherein the CDRs are as defined above. In one embodiment, the ISVD specifically binding to PD-1 comprises or consists of the amino acid sequence of SEQ ID NO: 439.

In another embodiment, the ISVD specifically binding to human PD-1 may have a sequence identity of more than 90%, such as more than 95% or even more than 99%, with SEQ ID NO: 440, wherein the CDRs are as defined above. In one embodiment, the ISVD specifically binding to PD-1 comprises or consists of the amino acid sequence of SEQ ID NO: 440.

In another embodiment, the ISVD specifically binding to human PD-1 may have a sequence identity of more than 90%, such as more than 95% or even more than 99%, with SEQ ID NO: 441, wherein the CDRs are as defined above. In one embodiment, the ISVD specifically binding to PD-1 comprises or consists of the amino acid sequence of SEQ ID NO: 441.

In another embodiment, the ISVD specifically binding to human PD-1 may have a sequence identity of more than 90%, such as more than 95% or even more than 99%, with SEQ ID NO: 442, wherein the CDRs are as defined above. In one embodiment, the ISVD specifically binding to PD-1 comprises or consists of the amino acid sequence of SEQ ID NO: 442.

In another embodiment, the ISVD specifically binding to human PD-1 may have a sequence identity of more than 90%, such as more than 95% or even more than 99%, with SEQ ID NO: 443, wherein the CDRs are as defined above. In one embodiment, the ISVD specifically binding to PD-1 comprises or consists of the amino acid sequence of SEQ ID NO: 443.

In another embodiment, the ISVD specifically binding to human PD-1 may have a sequence identity of more than 90%, such as more than 95% or even more than 99%, with SEQ ID NO: 444, wherein the CDRs are as defined above. In one embodiment, the ISVD specifically binding to PD-1 comprises or consists of the amino acid sequence of SEQ ID NO: 444.

In another embodiment, the ISVD specifically binding to human PD-1 may have a sequence identity of more than 90%, such as more than 95% or even more than 99%, with SEQ ID NO: 445, wherein the CDRs are as defined above. In one embodiment, the ISVD specifically binding to PD-1 comprises or consists of the amino acid sequence of SEQ ID NO: 445.

In another embodiment, the ISVD specifically binding to human PD-1 may have a sequence identity of more than 90%, such as more than 95% or even more than 99%, with SEQ ID NO: 446, wherein the CDRs are as defined above. In one embodiment, the ISVD specifically binding to PD-1 comprises or consists of the amino acid sequence of SEQ ID NO: 446.

In another embodiment, the ISVD specifically binding to human PD-1 may have a sequence identity of more than 90%, such as more than 95% or even more than 99%, with SEQ ID NO: 447, wherein the CDRs are as defined above. In one embodiment, the ISVD specifically binding to PD-1 comprises or consists of the amino acid sequence of SEQ ID NO: 447.

In another embodiment, the ISVD specifically binding to human PD-1 may have a sequence identity of more than 90%, such as more than 95% or even more than 99%, with SEQ ID NO: 448, wherein the CDRs are as defined above. In one embodiment, the ISVD specifically binding to PD-1 comprises or consists of the amino acid sequence of SEQ ID NO: 448.

In another embodiment, the ISVD specifically binding to human PD-1 may have a sequence identity of more than 90%, such as more than 95% or even more than 99%, with SEQ ID NO: 449, wherein the CDRs are as defined above. In one embodiment, the ISVD specifically binding to PD-1 comprises or consists of the amino acid sequence of SEQ ID NO: 449.

In another embodiment, the ISVD specifically binding to human PD-1 may have a sequence identity of more than 90%, such as more than 95% or even more than 99%, with SEQ ID NO: 450, wherein the CDRs are as defined above. In one embodiment, the ISVD specifically binding to PD-1 comprises or consists of the amino acid sequence of SEQ ID NO: 450.

In another embodiment, the ISVD specifically binding to human PD-1 may have a sequence identity of more than 90%, such as more than 95% or even more than 99%, with SEQ ID NO: 451, wherein the CDRs are as defined above. In one embodiment, the ISVD specifically binding to PD-1 comprises or consists of the amino acid sequence of SEQ ID NO: 451.

In another embodiment, the ISVD specifically binding to human PD-1 may have a sequence identity of more than 90%, such as more than 95% or even more than 99%, with SEQ ID NO: 452, wherein the CDRs are as defined above. In one embodiment, the ISVD specifically binding to PD-1 comprises or consists of the amino acid sequence of SEQ ID NO: 452.

In another embodiment, the ISVD specifically binding to human PD-1 may have a sequence identity of more than 90%, such as more than 95% or even more than 99%, with SEQ ID NO: 453, wherein the CDRs are as defined above. In one embodiment, the ISVD specifically binding to PD-1 comprises or consists of the amino acid sequence of SEQ ID NO: 453.

In another embodiment, the ISVD specifically binding to human PD-1 may have a sequence identity of more than 90%, such as more than 95% or even more than 99%, with SEQ ID NO: 454, wherein the CDRs are as defined above. In one embodiment, the ISVD specifically binding to PD-1 comprises or consists of the amino acid sequence of SEQ ID NO: 454.

In another embodiment, the ISVD specifically binding to human PD-1 may have a sequence identity of more than 90%, such as more than 95% or even more than 99%, with SEQ ID NO: 455, wherein the CDRs are as defined above. In one embodiment, the ISVD specifically binding to PD-1 comprises or consists of the amino acid sequence of SEQ ID NO: 455.

In another embodiment, the ISVD specifically binding to human PD-1 may have a sequence identity of more than 90%, such as more than 95% or even more than 99%, with SEQ ID NO: 456, wherein the CDRs are as defined above. In one embodiment, the ISVD specifically binding to PD-1 comprises or consists of the amino acid sequence of SEQ ID NO: 456.

In another embodiment, the ISVD specifically binding to human PD-1 may have a sequence identity of more than 90%, such as more than 95% or even more than 99%, with SEQ ID NO: 457, wherein the CDRs are as defined above. In one embodiment, the ISVD specifically binding to PD-1 comprises or consists of the amino acid sequence of SEQ ID NO: 457.

In another embodiment, the ISVD specifically binding to human PD-1 may have a sequence identity of more than 90%, such as more than 95% or even more than 99%, with SEQ ID NO: 458, wherein the CDRs are as defined above. In one embodiment, the ISVD specifically binding to PD-1 comprises or consists of the amino acid sequence of SEQ ID NO: 458.

In another embodiment, the ISVD specifically binding to human PD-1 may have a sequence identity of more than 90%, such as more than 95% or even more than 99%, with SEQ ID NO: 459, wherein the CDRs are as defined above. In one embodiment, the ISVD specifically binding to PD-1 comprises or consists of the amino acid sequence of SEQ ID NO: 459.

In another embodiment, the ISVD specifically binding to human PD-1 may have a sequence identity of more than 90%, such as more than 95% or even more than 99%, with SEQ ID NO: 460, wherein the CDRs are as defined above. In one embodiment, the ISVD specifically binding to PD-1 comprises or consists of the amino acid sequence of SEQ ID NO: 460.

In another embodiment, the ISVD specifically binding to human PD-1 may have a sequence identity of more than 90%, such as more than 95% or even more than 99%, with SEQ ID NO: 461, wherein the CDRs are as defined above. In one embodiment, the ISVD specifically binding to PD-1 comprises or consists of the amino acid sequence of SEQ ID NO: 461.

In another embodiment, the ISVD specifically binding to human PD-1 may have a sequence identity of more than 90%, such as more than 95% or even more than 99%, with SEQ ID NO: 462, wherein the CDRs are as defined above. In one embodiment, the ISVD specifically binding to PD-1 comprises or consists of the amino acid sequence of SEQ ID NO: 462.

In another embodiment, the ISVD specifically binding to human PD-1 may have a sequence identity of more than 90%, such as more than 95% or even more than 99%, with SEQ ID NO: 463, wherein the CDRs are as defined above. In one embodiment, the ISVD specifically binding to PD-1 comprises or consists of the amino acid sequence of SEQ ID NO: 463.

In another embodiment, the ISVD specifically binding to human PD-1 may have a sequence identity of more than 90%, such as more than 95% or even more than 99%, with SEQ ID NO: 464, wherein the CDRs are as defined above. In one embodiment, the ISVD specifically binding to PD-1 comprises or consists of the amino acid sequence of SEQ ID NO: 464.

In another embodiment, the ISVD specifically binding to human PD-1 may have a sequence identity of more than 90%, such as more than 95% or even more than 99%, with SEQ ID NO: 465, wherein the CDRs are as defined above. In one embodiment, the ISVD specifically binding to PD-1 comprises or consists of the amino acid sequence of SEQ ID NO: 465.

In another embodiment, the ISVD specifically binding to human PD-1 may have a sequence identity of more than 90%, such as more than 95% or even more than 99%, with SEQ ID NO: 466, wherein the CDRs are as defined above. In one embodiment, the ISVD specifically binding to PD-1 comprises or consists of the amino acid sequence of SEQ ID NO: 466.

In another embodiment, the ISVD specifically binding to human PD-1 may have a sequence identity of more than 90%, such as more than 95% or even more than 99%, with SEQ ID NO: 467, wherein the CDRs are as defined above. In one embodiment, the ISVD specifically binding to PD-1 comprises or consists of the amino acid sequence of SEQ ID NO: 467.

In another embodiment, the ISVD specifically binding to human PD-1 may have a sequence identity of more than 90%, such as more than 95% or even more than 99%, with SEQ ID NO: 468, wherein the CDRs are as defined above. In one embodiment, the ISVD specifically binding to PD-1 comprises or consists of the amino acid sequence of SEQ ID NO: 468.

In another embodiment, the ISVD specifically binding to human PD-1 may have a sequence identity of more than 90%, such as more than 95% or even more than 99%, with SEQ ID NO: 469, wherein the CDRs are as defined above. In one embodiment, the ISVD specifically binding to PD-1 comprises or consists of the amino acid sequence of SEQ ID NO: 469.

In another embodiment, the ISVD specifically binding to human PD-1 may have a sequence identity of more than 90%, such as more than 95% or even more than 99%, with SEQ ID NO: 470, wherein the CDRs are as defined above. In one embodiment, the ISVD specifically binding to PD-1 comprises or consists of the amino acid sequence of SEQ ID NO: 470.

In another embodiment, the ISVD specifically binding to human PD-1 may have a sequence identity of more than 90%, such as more than 95% or even more than 99%, with SEQ ID NO: 471, wherein the CDRs are as defined above. In one embodiment, the ISVD specifically binding to PD-1 comprises or consists of the amino acid sequence of SEQ ID NO: 471.

In another embodiment, the ISVD specifically binding to human PD-1 may have a sequence identity of more than 90%, such as more than 95% or even more than 99%, with SEQ ID NO: 472, wherein the CDRs are as defined above. In one embodiment, the ISVD specifically binding to PD-1 comprises or consists of the amino acid sequence of SEQ ID NO: 472.

In another embodiment, the ISVD specifically binding to human PD-1 may have a sequence identity of more than 90%, such as more than 95% or even more than 99%, with SEQ ID NO: 473, wherein the CDRs are as defined above. In one embodiment, the ISVD specifically binding to PD-1 comprises or consists of the amino acid sequence of SEQ ID NO: 473.

In another embodiment, the ISVD specifically binding to human PD-1 may have a sequence identity of more than 90%, such as more than 95% or even more than 99%, with SEQ ID NO: 474, wherein the CDRs are as defined above. In one embodiment, the ISVD specifically binding to PD-1 comprises or consists of the amino acid sequence of SEQ ID NO: 474.

In another embodiment, the ISVD specifically binding to human PD-1 may have a sequence identity of more than 90%, such as more than 95% or even more than 99%, with SEQ ID NO: 475, wherein the CDRs are as defined above. In one embodiment, the ISVD specifically binding to PD-1 comprises or consists of the amino acid sequence of SEQ ID NO: 475.

In another embodiment, the ISVD specifically binding to human PD-1 may have a sequence identity of more than 90%, such as more than 95% or even more than 99%, with SEQ ID NO: 476, wherein the CDRs are as defined above. In one embodiment, the ISVD specifically binding to PD-1 comprises or consists of the amino acid sequence of SEQ ID NO: 476.

In another embodiment, the ISVD specifically binding to human PD-1 may have a sequence identity of more than 90%, such as more than 95% or even more than 99%, with SEQ ID NO: 477, wherein the CDRs are as defined above. In one embodiment, the ISVD specifically binding to PD-1 comprises or consists of the amino acid sequence of SEQ ID NO: 477.

In another embodiment, the ISVD specifically binding to human PD-1 may have a sequence identity of more than 90%, such as more than 95% or even more than 99%, with SEQ ID NO: 478, wherein the CDRs are as defined above. In one embodiment, the ISVD specifically binding to PD-1 comprises or consists of the amino acid sequence of SEQ ID NO: 478.

In another embodiment, the ISVD specifically binding to human PD-1 may have a sequence identity of more than 90%, such as more than 95% or even more than 99%, with SEQ ID NO: 479, wherein the CDRs are as defined above. In one embodiment, the ISVD specifically binding to PD-1 comprises or consists of the amino acid sequence of SEQ ID NO: 479.

In another embodiment, the ISVD specifically binding to human PD-1 may have a sequence identity of more than 90%, such as more than 95% or even more than 99%, with SEQ ID NO: 480, wherein the CDRs are as defined above. In one embodiment, the ISVD specifically binding to PD-1 comprises or consists of the amino acid sequence of SEQ ID NO: 480.

In another embodiment, the ISVD specifically binding to human PD-1 may have a sequence identity of more than 90%, such as more than 95% or even more than 99%, with SEQ ID NO: 481, wherein the CDRs are as defined above. In one embodiment, the ISVD specifically binding to PD-1 comprises or consists of the amino acid sequence of SEQ ID NO: 481.

In another embodiment, the ISVD specifically binding to human PD-1 may have a sequence identity of more than 90%, such as more than 95% or even more than 99%, with SEQ ID NO: 482, wherein the CDRs are as defined above. In one embodiment, the ISVD specifically binding to PD-1 comprises or consists of the amino acid sequence of SEQ ID NO: 482.

In another embodiment, the ISVD specifically binding to human PD-1 may have a sequence identity of more than 90%, such as more than 95% or even more than 99%, with SEQ ID NO: 483, wherein the CDRs are as defined above. In one embodiment, the ISVD specifically binding to PD-1 comprises or consists of the amino acid sequence of SEQ ID NO: 483.

In another embodiment, the ISVD specifically binding to human PD-1 may have a sequence identity of more than 90%, such as more than 95% or even more than 99%, with SEQ ID NO: 484, wherein the CDRs are as defined above. In one embodiment, the ISVD specifically binding to PD-1 comprises or consists of the amino acid sequence of SEQ ID NO: 484.

In another embodiment, the ISVD specifically binding to human PD-1 may have a sequence identity of more than 90%, such as more than 95% or even more than 99%, with SEQ ID NO: 485, wherein the CDRs are as defined above. In one embodiment, the ISVD specifically binding to PD-1 comprises or consists of the amino acid sequence of SEQ ID NO: 485.

In another embodiment, the ISVD specifically binding to human PD-1 may have a sequence identity of more than 90%, such as more than 95% or even more than 99%, with SEQ ID NO: 486, wherein the CDRs are as defined above. In one embodiment, the ISVD specifically binding to PD-1 comprises or consists of the amino acid sequence of SEQ ID NO: 486.

In another embodiment, the ISVD specifically binding to human PD-1 may have a sequence identity of more than 90%, such as more than 95% or even more than 99%, with SEQ ID NO: 487, wherein the CDRs are as defined above. In one embodiment, the ISVD specifically binding to PD-1 comprises or consists of the amino acid sequence of SEQ ID NO: 487.

In another embodiment, the ISVD specifically binding to human PD-1 may have a sequence identity of more than 90%, such as more than 95% or even more than 99%, with SEQ ID NO: 488, wherein the CDRs are as defined above. In one embodiment, the ISVD specifically binding to PD-1 comprises or consists of the amino acid sequence of SEQ ID NO: 488.

In another embodiment, when such an ISVD specifically binding to PD-1 has 3, 2 or 1 amino acid difference in at least one CDR relative to a corresponding reference CDR sequence (above), the ISVD has at least (essentially) the same binding affinity to human PD-1 compared to one of the VHHs with SEQ ID NOs: 1-488 (preferably 402, 331, 332, 375, 212, 139, 238), wherein the binding affinity is measured using the same method, such as e.g. SPR.

In another embodiment, when such an ISVD specifically binding to PD-1 has 3, 2 or 1 amino acid difference in at least one CDR relative to a corresponding reference CDR sequence (above), the ISVD has at least (essentially) the same binding affinity to cyno PD-1 compared to one of the VHHs with SEQ ID NOs: 1-488 (preferably 402, 331, 332, 375, 212, 139, 238), wherein the binding affinity is measured using the same method, such as e.g. SPR.

In another embodiment, when such an ISVD specifically binding to PD-1 has 3, 2 or 1 amino acid difference in at least one CDR relative to a corresponding reference CDR sequence (above), the ISVD still specifically binds to human PD-1 and cyno PD-1 with less than 10-fold difference in affinity (K_{D}).

In another embodiment, when such an ISVD specifically binding to PD-1 has 3, 2 or 1 amino acid difference in at least one CDR relative to a corresponding reference CDR sequence (above), the ISVD still specifically binds to human and cyno PD-1 and does not bind to other members of the CD28 receptor family.

In another embodiment, when such an ISVD specifically binding to PD-1 has 3, 2 or 1 amino acid difference in at least one CDR relative to a corresponding reference CDR sequence (above), the ISVD still specifically binds to human and cyno PD-1 and does not bind to CD28, CTLA-4, ICOS, and BTLA. In another embodiment, when such an ISVD specifically binding to PD-1 has 3, 2 or 1 amino acid difference in at least one CDR relative to a corresponding reference CDR sequence (above), the ISVD blocks the interaction of human PD-1 with human PD-L1 with at least (essentially) the same potency compared to one of the VHHs with SEQ ID NOs: 1-488 (preferably 402, 331, 332, 375, 212, 139, 238), wherein the potency is measured, e.g., in FACS competition assay on CHO cells expressing human PD-1, with hPD-L1 as competitor.

In another embodiment, when such an ISVD specifically binding to PD-1 has 3, 2 or 1 amino acid difference in at least one CDR relative to a corresponding reference CDR sequence (above), the ISVD blocks the interaction of cyno PD-1 with cyno PD-L1 with at least (essentially) the same potency compared to one of the VHHs with SEQ ID NOs: 1-488 (preferably 402, 331, 332, 375, 212, 139, 238), wherein the potency is measured, e.g., in FACS competition assay on CHO cells expressing cyno PD-1, with hPD-L1 as competitor.

In another embodiment, when such an ISVD specifically binding to PD-1 has 3, 2 or 1 amino acid difference in at least one CDR relative to a corresponding reference CDR sequence (above), the ISVD blocks the interaction of hPD-1 with PD-L1 with (essentially) the same % inhibition or more compared to one of the VHHs with SEQ ID NOs: 1-488 (preferably 402, 331, 332, 375, 212, 139, 238), wherein the % inhibition is determined, e.g., in a FACS competition assay.

In another embodiment, when such an ISVD specifically binding to PD-1 has 3, 2 or 1 amino acid difference in at least one CDR relative to a corresponding reference CDR sequence (above), the ISVD has at least (essentially) the same potency for T-cell activation compared to one of the VHHs with SEQ ID NOs: 1-488 (preferably 402, 331, 332, 375, 212, 139, 238), wherein the potency is monitored, e.g., via IFN-gamma production in an autologous Tetanus Toxoid recall assay (TT assay), e.g. the TT assay as described herein.

In another embodiment, when such an ISVD specifically binding to PD-1 has 3, 2 or 1 amino acid difference in at least one CDR relative to a corresponding reference CDR sequence (above), the ISVD has at least (essentially) the same efficacy for T-cell activation compared to one of the VHHs with SEQ ID NOs: 1-488 (preferably 402, 331, 332, 375, 212, 139, 238), wherein the efficacy is monitored, e.g., via IFN-gamma production in an autologous Tetanus Toxoid recall assay (TT assay), e.g. the TT assay as described herein

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same binding affinity to human PD-1 compared to one of the VHHs with SEQ ID NOs: 1-488 (preferably 402, 331, 332, 375, 212, 139, 238), wherein the binding affinity is measured using the same method, such as e.g. SPR.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same binding affinity to cyno PD-1 compared to one of the VHHs with SEQ ID NOs: 1-488 (preferably 402, 331, 332, 375, 212, 139, 238), wherein the binding affinity is measured using the same method, such as e.g. SPR.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human PD-1 and cyno PD-1 with less than 10-fold difference in affinity (K_{D}).

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human and cyno PD-1 and does not bind to other members of the CD28 receptor family.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human and cyno PD-1 and does not bind to CD28, CTLA-4, ICOS, and BTLA. In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of human PD-1 with human PD-L1 with at least (essentially) the same potency compared to one of the VHHs with SEQ ID NOs: 1-488 (preferably 402, 331, 332, 375, 212, 139, 238), wherein the potency is measured, e.g., in FACS competition assay on CHO cells expressing human PD-1, with hPD-L1 as competitor

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of cyno PD-1 with cyno PD-L1 with at least (essentially) the same potency compared to one of the VHHs with SEQ ID NOs: 1-488 (preferably 402, 331, 332, 375, 212, 139, 238), wherein the potency is measured, e.g., in FACS competition assay on CHO cells expressing cyno PD-1, with hPD-L1 as competitor

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of hPD-1 with (h) PD-L1 with (essentially) the same % inhibition or more compared to one of the VHHs with SEQ ID NOs: 1-488 (preferably 402, 331, 332, 375, 212, 139, 238), wherein the % inhibition is determined, e.g., in a FACS competition assay.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same potency for T-cell activation compared to one of the VHHs with SEQ ID NOs: 1-488 (preferably 402, 331, 332, 375, 212, 139, 238), wherein the potency is monitored, e.g., via IFN-gamma production in an autologous Tetanus Toxoid recall assay (TT assay), e.g. the TT assay as described herein.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same efficacy for T-cell activation compared to one of the VHHs with SEQ ID NOs: 1-488 (preferably 402, 331, 332, 375, 212, 139, 238), wherein the efficacy is monitored, e.g., via IFN-gamma production in an autologous Tetanus Toxoid recall assay (TT assay), e.g. the TT assay as described herein.

In another embodiment, when the ISVD specifically binding to human PD-1 has a sequence identity of more than 90%, such as more than 95% or more than 99%, with one of the VHHs with SEQ ID NOs: 1-488, the ISVD has at least (essentially) the same binding affinity to human PD-1 compared to one of the VHHs with SEQ ID NOs: 1-488 (preferably 402, 331, 332, 375, 212, 139, 238), wherein the binding affinity is measured using the same method, such as e.g. SPR.

In another embodiment, when the ISVD specifically binding to human PD-1 has a sequence identity of more than 90%, such as more than 95% or more than 99%, with one of the VHHs with SEQ ID NOs: 1-488, the ISVD has at least (essentially) the same binding affinity to cyno PD-1 compared to one of the VHHs with SEQ ID NOs: 1-488 (preferably 402, 331, 332, 375, 212, 139, 238), wherein the binding affinity is measured using the same method, such as e.g. SPR.

In another embodiment, when the ISVD specifically binding to human PD-1 has a sequence identity of more than 90%, such as more than 95% or more than 99%, with one of the VHHs with SEQ ID NOs: 1-488, the ISVD still specifically binds to human PD-1 and cyno PD-1 with less than 10-fold difference in affinity (K_{D}).

In another embodiment, when the ISVD specifically binding to human PD-1 has a sequence identity of more than 90%, such as more than 95% or more than 99%, with one of the VHHs with SEQ ID NOs: 1-488, the ISVD still specifically binds to human and cyno PD-1 and does not bind to other members of the CD28 receptor family.

In another embodiment, when the ISVD specifically binding to human PD-1 has a sequence identity of more than 90%, such as more than 95% or more than 99%, with one of the VHHs with SEQ ID NOs: 1-488, the ISVD still specifically binds to human and cyno PD-1 and does not bind to CD28, CTLA-4, ICOS, and BTLA.

In another embodiment, when the ISVD specifically binding to human PD-1 has a sequence identity of more than 90%, such as more than 95% or more than 99%, with one of the VHHs with SEQ ID NOs: 1-488, the ISVD blocks the interaction of human PD-1 with human PD-L1 with at least (essentially) the same potency compared to one of the VHHs with SEQ ID NOs: 1-488 (preferably 402, 331, 332, 375, 212, 139, 238), wherein the potency is measured in wherein the potency is measured, e.g., in FACS competition assay on CHO cells expressing human PD-1, with hPD-L1 as competitor.

In another embodiment, when the ISVD specifically binding to human PD-1 has a sequence identity of more than 90%, such as more than 95% or more than 99%, with one of the VHHs with SEQ ID NOs: 1-488, the ISVD blocks the interaction of cyno PD-1 with cyno PD-L1 with at least (essentially) the same potency compared to one of the VHHs with SEQ ID NOs: 1-488 (preferably 402, 331, 332, 375, 212, 139, 238), wherein the potency is measured, e.g., in FACS competition assay on CHO cells expressing cyno PD-1, with hPD-L1 as competitor.

In another embodiment, when the ISVD specifically binding to human PD-1 has a sequence identity of more than 90%, such as more than 95% or more than 99%, with one of the VHHs with SEQ ID NOs: 1-488, the ISVD blocks the interaction of hPD-1 with (h) PD-L1 with (essentially) the same % inhibition or more compared to one of the VHHs with SEQ ID NOs: 1-488 (preferably 402, 331, 332, 375, 212, 139, 238), wherein the % inhibition is determined, e.g., in a FACS competition assay.

In another embodiment, when the ISVD specifically binding to human PD-1 has a sequence identity of more than 90%, such as more than 95% or more than 99%, with one of the VHHs with SEQ ID NOs: 1-488, the ISVD has at least (essentially) half of the potency, preferably at least (essentially) the same potency for T-cell activation compared to one of the VHHs with SEQ ID NOs: 1-488 (preferably 402, 331, 332, 375, 212, 139, 238), wherein the potency is monitored, e.g., via IFN-gamma production in an autologous Tetanus Toxoid recall assay (TT assay), e.g. the TT assay as described herein.

In another embodiment, when the ISVD specifically binding to human PD-1 has a sequence identity of more than 90%, such as more than 95% or more than 99%, with one of the VHHs with SEQ ID NOs: 1-488, the ISVD has at least 75% of the efficacy, preferably at least 80% or 90% of the efficacy, more preferably at least (essentially) the same efficacy T-cell activation compared to one of the VHHs with SEQ ID NOs: 1-488 (preferably 402, 331, 332, 375, 212, 139, 238), wherein the efficacy is monitored, e.g., via IFN-gamma production in an autologous Tetanus Toxoid recall assay (TT assay), e.g. the TT assay as described herein.

In some embodiments, the immunoglobulin single variable domains of the present technology specifically bind to human PD-1 with a dissociation constant (K_{D}) of 10⁻⁹ moles/litre or less, 10⁻¹⁰ moles/litre or less, such as 5×10⁻⁹ to 10⁻¹² moles/litre or less, and preferably 10⁻¹⁰ to 10⁻¹² moles/litre or less and more preferably 5×10⁻¹⁰ to 10⁻¹¹ moles/litre, 5×10⁻¹⁰ to 5×10⁻¹¹ moles/litre, 10⁻¹⁰ to 10⁻¹² moles/litre, or 10⁻¹⁰ to 10⁻¹¹ moles/litre, such as 4.3×10⁻¹¹ moles/litre, as determined by Surface Plasmon Resonance, e.g., measured at 25° C.

In some embodiments, the immunoglobulin single variable domains of the present technology specifically bind to human PD-1 with a kₒₙ-rate of between 10⁵ M⁻¹ s⁻¹ to about 10⁷ M⁻¹ s⁻¹, preferably between 5×10⁵ M⁻¹ s⁻¹ and 10⁷ M⁻¹ s⁻¹, more preferably between 10⁵ M⁻¹ s⁻¹ and 5×10⁶ M⁻¹ s⁻¹, such as between 10⁵ M⁻¹ s⁻¹ and 1×10⁶ M⁻¹ s⁻¹, such as 3×10⁵ M⁻¹ s⁻¹, as determined by Surface Plasmon Resonance, e.g., measured at 25° C.

In some embodiments, the immunoglobulin single variable domains of the present technology specifically bind to human PD-1 with a k_{off} rate between 10⁻³ s⁻¹ (t1/2=0.69 s) and 10⁻⁶ s⁻¹ (providing a near irreversible complex with a t1/2 of multiple days), preferably between 10⁻³ s⁻¹ and 5×10⁻⁶ s⁻¹, more preferably between 5×10⁻⁴ s⁻¹ and 5×10⁶ s⁻¹, such as between 5×10⁻⁴ s⁻¹ and 10⁻⁵ s⁻¹, such as 1.3×10⁻⁵ s⁻¹, as determined by Surface Plasmon Resonance, e.g., measured at 25° C.

In some embodiments, the immunoglobulin single variable domains of the present technology specifically bind to cyno PD-1 with a dissociation constant (K_{D}) of 10⁻⁹ to 10⁻¹² moles/litre or less, 5×10⁹ to 10⁻¹² moles/litre or less, and preferably 10⁻¹⁰ to 10⁻¹² moles/litre or less and more preferably 10⁻¹⁰ to 10⁻¹¹ moles/litre, such as 2.9×10⁻¹¹ moles/litre, as determined by Surface Plasmon Resonance, e.g., measured at 25° C.

In some embodiments, the immunoglobulin single variable domains of the present technology specifically bind to cyno PD-1 with a kₒₙ-rate of between 10⁴ M⁻¹ s⁻¹ to about 10⁷ M⁻¹ s⁻¹, preferably between 5×10⁴ M⁻¹ s⁻¹ and 10⁷ M⁻¹ s⁻¹, more preferably between 10⁵ M⁻¹ s⁻¹ and 10⁷ M⁻¹ s⁻¹, such as between 10⁵ M⁻¹ s⁻¹ and 10⁶ M⁻¹ s⁻¹, such as 2.8×10⁵ M⁻¹ s⁻¹, as determined by Surface Plasmon Resonance, e.g., measured at 25° C.

In some embodiments, the immunoglobulin single variable domains of the present technology specifically bind to cyno PD-1 with a k_{off} rate between 10⁻³ s⁻¹ (t1/2=0.69 s) and 10⁻⁸ s⁻¹ (providing a near irreversible complex with a t1/2 of multiple days), preferably between 10⁻³ s⁻¹ and 5×10⁻⁷ s⁻¹, more preferably between 5×10⁻⁴ s⁻¹ and 10⁻⁷ s⁻¹, such as between 10⁻⁵ s⁻¹ and 10⁻⁷ s⁻¹, such as 8.2×10⁶ s⁻¹, as determined by Surface Plasmon Resonance, e.g., measured at 25° C.

In some embodiments, the immunoglobulin single variable domains of the present technology specifically bind to human PD-1 and cyno PD-1 with less than 10-fold difference in affinity (K_{D}). In some embodiments, the immunoglobulin single variable domains of the present technology specifically bind to human and cyno PD-1 and do not bind to other members of the CD28 receptor family.

In some embodiments, the immunoglobulin single variable domains of the present technology specifically bind to human and cyno PD-1 and do not bind to CD28, CTLA-4, ICOS, and BTLA.

In the present technology, the binding of the ISVDs and polypeptides comprising the ISVD to PD-1 can also be measured in assays in which PD-1 is exposed on a cell surface (such as e.g. CHO Flp-In human PD-1 cells, CHO Flp-In cyno PD-1 cells). The Invitrogen™ Flp-In™ System allows integration and expression of the gene of interest (e.g. encoding human, cyno or mouse PD-1) in mammalian cells (e.g. CHO cells) at a specific genomic location. The Flp-In System involves introduction of a Flp Recombination Target (FRT) site into the genome of the mammalian cell line of choice. An expression vector containing the gene of interest is then integrated into the genome via Flp recombinase-mediated DNA recombination at the FRT site (O'Gorman et al., 1991, Recombinase-Mediated Gene Activation and Site-Specific Integration in Mammalian Cells, Science 251, 1351-1355).

A preferred assay for measuring binding of the ISVD (or polypeptide comprising the ISVD) to PD-1 exposed on a cell surface is a FACS assay, such as e.g. the FACS assay as described in the examples, wherein binding of the ISVDs or polypeptides to PD-1 expressed on CHO is determined.

In some embodiments, the immunoglobulin single variable domains of the present technology specifically bind PD-1 and block the interaction of PD-1 with PD-L1. In some embodiments, the immunoglobulin single variable domains of the present technology specifically bind hPD-1 and block the interaction of hPD-1 with hPD-L1. In some embodiments, the immunoglobulin single variable domains of the present technology specifically bind cyno PD-1 and block the interaction of cyno PD-1 with cyno PD-L1.

In some embodiments, the immunoglobulin single variable domains of the present technology show a highly potency and/or efficacy (as a single variable domain) for binding PD-1 in competition with PD-L1. In some embodiments, the immunoglobulin single variable domains of the present technology can functionally block PD-1. More specifically, the ISVD can block the interaction between PD-1 and PD-L1. In some embodiments, the immunoglobulin single variable domains of the present technology antagonize an activity of PD-1.

In some embodiments, the immunoglobulin single variable domains of the present technology specifically bind to human PD-1 and block the interaction of human PD-1 with human PD-L1. For example, in FACS competition assay on CHO cells expressing human PD-1, with hPD-L1 as competitor, the immunoglobulin single variable domains of the present technology may have IC50 values of 10⁻⁸ M or lower, more preferably of 5×10⁻⁹ M or lower, or even of 10⁻⁹ M or lower. For example, in such FACS competition assay, the immunoglobulin single variable domains of the present technology may have IC50 values between 10⁻¹¹ M and 10⁻⁸ M, such as between 10⁻¹⁰ M and 10⁻⁹ M, between 10⁻¹⁰ M and 10⁻⁸ M or between 10⁻⁹ M and 10⁻⁸ M, such as about 5×10⁻¹¹ M or 5×10⁻⁹ M.

In some embodiments, the immunoglobulin single variable domains of the present technology specifically bind to cyno PD-1 and block the interaction of cyno PD-1 with cyno PD-L1 or human PD-L1. For example, in a FACS competition assay on CHO cells expressing cyno PD-1, with human PD-L1 as competitor, the immunoglobulin single variable domains of the present technology may have IC50 values of 10⁻⁷ M or lower, 5×10⁻⁸ M or lower, more preferably of 10⁻⁸ M or lower, or even of 5×10⁻⁹ M or lower. For example, in such FACS competition assay, the immunoglobulin single variable domains of the present technology may have IC50 values between 10⁻¹¹ M and 10⁻⁷ M, between 10⁻¹¹ M and 10⁻⁸ M, such as between 10⁻¹⁰ M and 10⁻⁹ M, between 10⁻¹⁰ M and 10⁻⁸ M between 10⁻⁹ M and 10⁻⁸ M or between 10⁻⁹ M and 10⁻⁷ M, such as about 5.76×10⁻⁹ M.

In some embodiments, the immunoglobulin single variable domains of the present technology block the binding of human PD-1 to human PD-L1 by at least 50%, such as at least 60%, 70%, 80%, 90%, 95% or even more, as determined by ligand competition, AlphaScreen, or competitive binding assays (such as competition ELISA or competition FACS).

In some embodiments, the immunoglobulin single variable domains of the present technology block the binding of cyno PD-1 to cyno PD-L1 by at least 50%, such as at least 60%, 70%, 80%, 90%, 95% or even more, as determined by ligand competition, AlphaScreen, or competitive binding assays (such as competition ELISA or competition FACS).

In some embodiments, the immunoglobulin single variable domains of the present technology show a highly potency for T cell activation as a single variable domain. T cell activation refers to a process in which mature T cells can express antigen-specific T cell receptors on their surface to recognize their cognate antigens and respond by entering the cell cycle, secreting cytokines or lytic enzymes, and initiating the cell-based functions of the immune system.

T-cell activation as used herein refers to one or more cellular response(s) of a T cell, e.g., a cytotoxic T cell, such as selected from: proliferation, differentiation, cytokine secretion, cytotoxic effector molecule release, cytotoxic activity, expression of activation markers, and redirected target cell lysis. The polypeptides of the invention are capable of inducing one or more of such responses. Suitable assays to measure T cell activation are known in the art, for instance as described in WO 99/54440 or by Schlereth et al. 2005 (Cancer Immunol. Immunother. 20:1-12), or as exemplified in the examples or below. For example, T cell activation can be measured by assessing (1) proliferation, (2) up-regulation of activation markers (e.g., IL2RA/CD25) and/or (3) production of effector cytokines (IFN-gamma, TNF-α).

In some embodiments, T cell activation by the ISVDs and polypeptides comprising the same can be monitored by upregulation of CD69, CD25 and various cell adhesion molecules, de novo expression and/or release of cytokines (e.g., IFN-γ, TNF-α, IL-6, IL-2, IL-4 and IL-10), upregulation of granzyme and perforin expression, and/or cell proliferation, membrane blebbing, activation of procaspases 3 and/or 7, fragmentation of nuclear DNA and/or cleavage of caspase substrate poly (ADPribose) polymerase.

In some embodiments, T cell activation is monitored by measurement of IFN-gamma release.

In some embodiments, T cell activation is measured by determining the expression of a number of molecules, namely CD25, CD69, CD71, and HLA-DR, also termed “activation antigens”, by activated proliferating lymphocytes on their surface.’ Such activation antigens are easily evidenced by immunofluorescence assay and flow cytometric analysis using monoclonal antibodies (mAbs) as specific reagents. By two- and three colour immunofluorescent staining it is also possible to identify, within bulk lymphocyte cultures, the T-cell subpopulations involved in the activation process.

In some embodiments, T cell activation can be measured by DNA synthesis assays. DNA synthesis assays use radioactively labelled 3H-thymine to detect DNA within cells. Newly proliferated cells incorporate the label, providing extremely sensitive and accurate detection of cell lines (see e.g., Madhavan 2007, J. Stem Cells Regen. Med. 3:12).

In some embodiments, T cell activation can be measured in a tetanus Toxoid recall assay (TT assay). The assay makes use of Lymphocytes that have previously been exposed to an antigen and that will show an enhanced response upon subsequent re-exposure. Primary lymphocytes from individuals vaccinated against tetanus are used to initiate an in vitro recall antigenic response. Stimulation of such lymphocytes with tetanus toxoid provides a useful assay method to assess the effects of candidate therapeutic molecules, such as checkpoint inhibitors, and as a model for analogous responses to tumor antigens.

Lymphocytes are antigenically stimulated following culture with loaded antigen presenting cells (APCs). Antigen stimulated lymphocytes are cultured in the presence of checkpoint inhibitors to modulate activation response. Endpoints that are assessed in this assay include lymphocyte proliferation, activation markers, and cytokine production (Ticha et al. 2021, A cell-based in vitro assay for testing of immunological integrity of Tetanus toxoid vaccine antigen, Npj Vaccines 6:1-11). In some embodiments, IFN-gamma production is measured following activation of the T lymphocytes.

In some embodiments, the immunoglobulin single variable domains of the present technology have a potency (EC50 values) in a TT assay (e.g. as described in the Examples), monitored via IFN-gamma production, of 10⁻⁸ M or lower, more preferably of 5×10⁻⁹ M or lower, or even of 10⁻⁹ M or lower. For example, in such TT assay, the immunoglobulin single variable domains of the present technology may have a potency (EC50 values) between 10⁻¹¹ M and 10⁻⁸ M, such as between 10⁻¹⁰ M and 10⁻⁸ M, between 10⁻¹⁰ M and 10⁻⁹ M or between 10⁻⁹ M and 10⁻⁸ M, such as 2.2×10⁻⁹ M, 2.8×10⁻⁹ M, or 3.2×10⁻⁹ M.

In some embodiments, a polypeptide comprising at least two immunoglobulin single variable domains of the present technology has a potency (EC50 values) in a TT assay (e.g. as described in the Examples), monitored via IFN-gamma production, of 10⁻⁸ M or lower, more preferably of 5×10⁻⁹ M or lower, or even of 10⁻⁹ M or lower. For example, in such TT assay, the immunoglobulin single variable domains of the present technology may have a potency (EC50 values) between 10⁻¹¹ M and 10⁻⁸ M, such as between 10⁻¹⁰ M and 10⁻⁸ M, between 10⁻¹⁰ M and 10⁻⁹ M or between 10⁻⁹ M and 10⁻⁸ M, such as 2.51×10⁻⁹ M, 2.0×10⁻⁹ M, 8.3×10⁻⁹ M, 2.6×10⁻⁹ M, or 7.5×10⁻¹⁰ M.

In some embodiments, the immunoglobulin single variable domains of the present technology have an efficacy in a TT assay (e.g., as described in the Examples), monitored via IFN-gamma production, of at least 50%, such as at least 60%, 70%, 80%, 90%, 95%.

The T Cell Activation Bioassay (NFAT) is a bioluminescent cell-based assay that consists of a genetically engineered Jurkat T cell line that expresses a luciferase reporter driven by an NFAT-response element (NFAT-RE). When the TCR/CD3 Effector Cells (NFAT) are engaged, the TCR transduces intracellular signals resulting in NFAT-RE-mediated luminescence. The bioluminescent signal is detected and quantified using Bio-Glo™ Luciferase Assay System and a standard luminometer, such as the GloMax® Discover System.

In some embodiments, the immunoglobulin single variable domains of the present technology have a potency (IC50 values) in a NFAT assay (e.g., as described in the Examples) of 10⁻⁷ M or lower, of 5×10⁻⁸ M or lower, more preferably of 10⁻⁸ M or lower, or even of 5×10⁻⁹ M or lower. For example, in such NFAT assay, the immunoglobulin single variable domains of the present technology may have a potency (IC50 values) between 10⁻¹⁰ M and 10⁻⁷ M, such as between 10⁻¹⁰ M and 10⁻⁸ M or between 10⁻¹⁰ M and 10⁻⁹ M, such as 1.56×10⁻⁹M or 1.2×10⁻⁹ M.

In some embodiments, the immunoglobulin single variable domains of the present technology block the binding of human PD-1 to human PD-L1 by at least 50%, such as at least 60%, 70%, 80%, 90%, 95% or even more, as determined in a NFAT assay.

In some embodiments, the immunoglobulin single variable domains of the present technology block the binding of human or cyno PD-L1 to cyno PD-1 by at least 50%, such as at least 60%, 70%, 80%, 90%, 95% or even more, as determined in a NFAT assay.

In another embodiment, when the ISVD specifically binding to human PD-1 has a sequence identity of more than 90%, such as more than 95% or more than 99%, with one of the VHHs with SEQ ID NOs: 1-488, the ISVD blocks the interaction of hPD-1 with (h) PD-L1 with (essentially) the same % inhibition or more compared to one of the VHHs with SEQ ID NOs: 1-488 (preferably, VHHs with SEQ ID NOs: 402, 331, 332, 375, 212, 139, 238), wherein the % inhibition is determined in a NFAT assay.

### 5.2.2 Embodiment 2, Family A0294041A11

There is provided an immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1 protein, wherein at least one amino acid residue located in at least one of the CDRs is capable of interacting with at least one amino acid residue in at least one, preferably in each, of the following stretches (of the ectodomain) of human PD-1 protein, when numbered in accordance with SEQ ID NO: 4703: N66-E84, and I126-E136, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.

In some embodiments, the at least one amino acid residue in at least one, preferably in each stretch (of the ectodomain) of human PD-1 protein is selected from:
For stretch N66-E84: N66, Y68, N74, Q75, T76, D77, K78, P83, and E84;
For stretch I126-E136: I126, L128, A129, P130, K131, A132, Q133, I134, and E136, when numbered in accordance with SEQ ID NO: 4703.

In some embodiments, at least one amino acid residue located in at least one of the CDRs is capable of interacting with at least two, at least three, at least four, at least five, at least six, at least seven, at least eight, or all of the following amino acid residues in stretch N66-E84 (of the ectodomain) of human PD-1 protein, when numbered in accordance with SEQ ID NO: 4703: N66, Y68, N74, Q75, T76, D77, K78, P83, and E84.

In some embodiments, at least one amino acid residue located in at least one of the CDRs is capable of interacting with at least two, at least three, at least four, at least five, at least six, at least seven, at least eight, or all of the following amino acid residues in stretch I126-E136 (of the ectodomain) of human PD-1 protein when numbered in accordance with SEQ ID NO: 4703: I126, L128, A129, P130, K131, A132, Q133, I134, and E136.

In some embodiments, at least one amino acid residue located in at least one of the CDRs is capable of forming salt bridges with at least the amino acid residue K78 of human PD-1 protein, when numbered in accordance with SEQ ID NO: 4703, optionally wherein at least one amino acid residue located in at least one of the CDRs is further capable of forming salt bridges with the amino acid residue E136 of human PD-1 protein, when numbered in accordance with SEQ ID NO: 4703, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.

In some embodiments, at least one amino acid residue located in at least one of the CDRs is capable of forming hydrogen bonds with at least one, preferably all, of the following amino acid residues of human PD-1 protein, when numbered in accordance with SEQ ID NO: 4703: N66, A129, P130, and K131, optionally wherein at least one amino acid residue located in at least one of the CDRs is further capable of forming hydrogen bonds with the amino acid residue N74 of human PD-1 protein, when numbered in accordance with SEQ ID NO: 4703, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.

In some embodiments, the at least one amino acid residue located in at least one of the CDRs is capable of interacting with an (discontinuous) epitope on human PD-1 comprising at least one amino acid residue in each of the following stretches (of the ectodomain) of human PD-1 protein: N66-E84, and I126-E136, when numbered in accordance with SEQ ID NO: 4703.

In some embodiments, the at least one amino acid residue in each stretch (of the ectodomain) of human PD-1 protein is selected from:
For stretch N66-E84: N66, Y68, N74, Q75, T76, D77, K78, P83, and E84;
For stretch I126-E136: I126, L128, A129, P130, K131, A132, Q133, I134, and E136, when numbered in accordance with SEQ ID NO: 4703.

In some embodiments, at least one amino acid residue located in at least one of the CDRs is capable of interacting with at least one, at least two, at least three, at least four, at least five, at least six, at least seven, at least ten, at least fifteen, preferably all, of the following amino acid residues of human PD-1 protein, when numbered in accordance with SEQ ID NO: 4703: N66, Y68, N74, Q75, T76, D77, K78, P83, E84, 1126, L128, A129, P130, K131, A132, Q133, I134, and E136, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.

In some embodiments, the amino acid residue located in at least one of the CDRs which are capable of interacting with amino acid residues of human PD-1 protein are located in CDR1 and/or CDR3.

In some embodiments, the:
amino acid at position 101 (Kabat numbering) is capable of interacting, preferably by forming a salt bridge, with K78 in PD-1; and/or
amino acid at position 99 (Kabat numbering) is capable of interacting, preferably by forming a salt bridge, with E136 in PD-1;
preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.

In some embodiments:
the amino acid residue at position 29 (Kabat numbering) is capable of interacting, preferably by forming a hydrogen bond, with A129 in PD-1;
the amino acid residue at position 30 (Kabat numbering) is capable of interacting, preferably by forming a hydrogen bond, with P130 in PD-1;
the amino acid residue at position 31 (Kabat numbering) is capable of interacting, preferably by forming a hydrogen bond, with P130 in PD-1;
the amino acid residue at position 32 (Kabat numbering) is capable of interacting, preferably by forming a hydrogen bond, with P130 in PD-1;
the amino acid residue at position 31 (Kabat numbering) is capable of interacting, preferably by forming a hydrogen bond, with K131 in PD-1;
the amino acid residue at position 101 (Kabat numbering) is capable of interacting, preferably by forming a hydrogen bond, with N66 in PD-1; and/or
the amino acid residue at position 99 (Kabat numbering) is capable of interacting, preferably by forming a hydrogen bond, with N74 in PD-1;
preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.

In some embodiments, two amino acids are considered to interact when the distance between them is 4.0 Å or less.

In some embodiments, the interacting amino acid residue(s) in the ISVD and in human PD-1 protein are identified by X-ray crystallography by:
Mixing human PD-1 protein, and the ISVD in PBS1× buffer;
Concentrating the protein complex to 8 mg/ml;
Crystallizing by hanging drop vapor diffusion using 100 mM Na Cacodylate, 300 mM Na Acetate, 10% PEG1000, pH 6.5 at 19° C.;
Collecting X-ray diffraction data;
Processing the collected data,
preferably as described in Example 13.

In some embodiments, the (discontinuous) epitope comprises or consists of the following amino acid residues of human PD-1 protein, when numbered in accordance with SEQ ID NO: 4703: N66, Y68, N74, Q75, T76, D77, K78, P83, E84, 1126, L128, A129, P130, K131, A132, Q133, I134, and E136.

In some embodiments, the ISVD cross-blocks the binding to human PD-1 protein of a polypeptide having an amino acid sequence selected from SEQ ID NOs: 714-1871, preferably the ISVD cross-blocks the binding to human PD-1 protein of a polypeptide having an amino acid sequence selected from SEQ ID NO: 1486, or the binding of the ISVD to human PD-1 protein is cross-blocked by a polypeptide having an amino acid sequence selected from SEQ ID NOs: 714-1871, preferably SEQ ID NO: 1486.

There is provided an immunoglobulin single variable domain (ISVD) specifically binding human PD-1 protein, that essentially consists of 4 framework regions (FR1 to FR4, respectively) and 3 complementarity determining regions (CDR1 to CDR3, respectively), wherein the ISVD comprises an amino acid residue capable of forming salt bridges at at least position 101 (Kabat numbering), and optionally further at position 99 (Kabat numbering), preferably wherein the ISVD comprises at least the following amino acid residue at the following positions: D101, and optionally K99 (Kabat numbering).

In some embodiments, the ISVD comprises an amino acid residue capable of forming hydrogen bonds at at least one, preferably at all, of the following positions: 29, 30, 31, 32, 101 (Kabat numbering) and optionally further at position 99 (Kabat numbering), preferably wherein the ISVD comprises at least one, preferably all of the following amino acid residues at the following positions: F29, S30, E31, N32, and D101 and optionally K99 (Kabat numbering).

In some embodiments, the ISVD comprises at least one, at least two, at least three, preferably all of the following amino acid residues at the following positions (Kabat numbering): D27, T28, F29, S30, E31, and N32, preferably at least F29, S30, E31, and N32.

In some embodiments, the ISVD comprises at least one, at least two, at least three, at least four, preferably all of the following amino acid residues at the following positions (Kabat numbering): 196, F97, K99, W100g, and D101, preferably at least K99 and D101.

In some embodiments:
the CDR1 (AbM definition) consists of the following amino acid sequence X₁DTFSENX2X₃X₄ (SEQ ID NO: 4694); wherein
   X₁ is selected from any amino acid, preferably X₁ is selected from G, K, F, A, Y, T, V, H, R, S, P, I, E, L, or D;
   X₂ is selected from any amino acid, preferably X₂ is selected from D, P, A, T, I, R, V, G, Y, L, F, K, S, I, H, or E;
   X₃ is selected from any amino acid, preferably X₃ is selected from I, R, H, M, G, Y, Q, E, K, F, T, S, A, D, or P;
   X₄ is selected from any amino acid, preferably X₄ is selected from G, A, L, S, R, H, V, P, Q, E, I, D, Y, T, or K; and/or
the CDR3 (AbM definition) consists of the following amino acid sequence X₁IF X₂KX₃X₄X₅X₆X₇X₈X₉WX₁₀X₁₁DX₁₂ (SEQ ID NO: 4695); wherein:
   X₁ is selected from any amino acid, preferably X₁ is selected from K, R, L, Y, T, I, Q, A, F, P, D, H, V, S, or E;
   X₂ is selected from any amino acid, preferably X₂ is selected from S, D, G, F, L, K, I, P, Y, V, A, or T;
   X₃ is selected from any amino acid, preferably X₃ is selected from T, K, A, F, S, G, D, P, H, I, Q, Y, L, or V;
   X₄ is selected from any amino acid, preferably X₄ is selected from G, F, H, Y, I, L, T, E, P, R, S, Q, D, K, or A;
   X₅ is selected from any amino acid, preferably X₅ is selected from T, E, G, V, S, Q, L, H, R, P, A, F, K, I, or Y;
   X₆ is selected from any amino acid, preferably X₆ is selected from Y, I, A, Q, G, E, F, D, K, L, S, T, R, or V;
   X₇ is selected from any amino acid, preferably X₇ is selected from S, T, G, L, Q, A, I, K, E, H, D, V, R, P, F, or Y;
   X₈ is selected from any amino acid, preferably X₈ is selected from A, H, Y, V, R, F, G, L, D, E, T, I, K, P, or S;
   X₉ is selected from any amino acid, preferably X₉ is selected from S, T, I, D, H, V, R, P, L, K, G, Y, V, E, F, or A;
   X₁₀ is selected from any amino acid, preferably X₁₀ is selected from E, R, P, I, T, A, F, D, S, H, V, K, L, G, Y, or Q;
   X₁₁ is selected from any amino acid, preferably X₁₁ is selected from Y, Q, L, G, V, P, K, E, I, R, H, D, or F; and
   X₁₂ is selected from any amino acid, preferably X₁₂ is selected from F, S, T, Y, K, R, A, H, G, I, V, D, Q, P, E, or L.

In some embodiments, the following amino acid residue of the ISVD at the following position: D101 (Kabat numbering) and optionally the following amino acid residue of the ISVD at the following position: K99 (Kabat numbering) are capable of forming salt bridges with at least one of the amino acid residues of human PD-1 protein, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.

In some embodiments, at least one, preferably all of the following amino acid residues of the ISVD at the following positions: F29, S30, E31, N32, and D101 (Kabat numbering) and optionally K99 (Kabat numbering) are capable of forming hydrogen bonds with at least one of the amino acid residues of human PD-1 protein, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.

In some embodiments, at least one, preferably all, of the following amino acid residues of the ISVD at the following positions: F29, S30, E31, N32, and D101 and optionally K99 (Kabat numbering) is capable of interacting with at least one, preferably all of the following amino acid residues of human PD-1 protein, when numbered in accordance with SEQ ID NO: 4703: N66, K78, A129, P130, and K131, and optionally N74, preferably wherein:
F29 in the ISVD (Kabat numbering) interacts, preferably by forming a hydrogen bond, with A129 in human PD-1 protein;
S30 in the ISVD (Kabat numbering) interacts, preferably by forming a hydrogen bond, with P130 in human PD-1 protein;
E31 in the ISVD (Kabat numbering) interacts, preferably by forming a hydrogen bond, with P130 in human PD-1 protein;
E31 in the ISVD (Kabat numbering) interacts, preferably by forming a hydrogen bond, with K131 in human PD-1 protein;
N32 in the ISVD (Kabat numbering) interacts, preferably by forming a hydrogen bond, with P130 in human PD-1 protein,
K99 in the ISVD (Kabat numbering) interacts, preferably by forming a hydrogen bond, with N74 in human PD-1 protein,
D101 in the ISVD (Kabat numbering) interacts, preferably by forming a hydrogen bond, with N66 in human PD-1 protein,
D101 in the ISVD (Kabat numbering) interacts, preferably by forming a salt bridge, with K78 in human PD-1 protein,
K99 in the ISVD (Kabat numbering) interacts, preferably by forming a salt bridge, with E136 in human PD-1 protein,
preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.

In some embodiments, the ISVD comprises at least one, at least two, at least three, at least four, at least five, preferably all of the following amino acid residues at the following positions (Kabat numbering): F29, S30, E31, N32, K99, and D101.

In some embodiments, the ISVD comprises at least one, at least two, at least three, at least four, at least five, at least six, at least seven, at least eight, at least nine, preferably all of the following amino acid residues at the following positions (Kabat numbering): D27, T28, F29, S30, E31, N32, 196, F97, K99, W100g, and D101.

In some embodiments, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 1957;
   b) amino acid sequences that have at least 80% amino acid identity with the amino acid sequence of SEQ ID NO: 1957; and
   c) amino acid sequences that have 3, 2, or 1 amino acid difference with the amino acid sequences of SEQ ID NO: 1957;
and
CDR3 (AbM definition) has an amino acid sequence selected from:
   a) the amino acid sequence of SEQ ID NOs: 2337;
   b) amino acid sequences that have at least 80% amino acid identity with the amino acid sequence of SEQ ID NO: 2337;
   c) amino acid sequences that have 3, 2, or 1 amino acid difference with the amino acid sequence of SEQ ID NO: 2337.

In some embodiments, the ISVD comprises D1 and/or V2 (Kabat numbering).

In some embodiments, the ISVD comprises at least one, at least two, at least three, at least four, at least five, at least six, at least seven, at least eight, at least nine, at least ten, at least eleven, at least twelve, preferably all of the following amino acid residues at the following positions (Kabat numbering): D1, V2, D27, T28, F29, S30, E31, N32, 196, F97, K99, W100g, and D101. In some embodiments, F29, S30, E31, N32, and D101 and optionally K99 (Kabat numbering) of the ISVD are capable of interacting with at least one of the following amino acids of human PD-1 protein, when numbered in accordance with SEQ ID NO: 4703: N66, Y68, N74, Q75, T76, D77, K78, P83, E84, 1126, L128, A129, P130, K131, A132, Q133, I134, and E136, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.

In some embodiments, D1, V2, D27, T28, F29, S30, E31, N32, 196, F97, K99, W100g, and D101 (Kabat numbering) of the ISVD are capable of interacting with the following amino acids of human PD-1 protein, when numbered in accordance with SEQ ID NO: 4703: N66, Y68, N74, Q75, T76, D77, K78, P83, E84, 1126, L128, A129, P130, K131, A132, Q133, I134, and E136, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.

In some embodiments, two amino acids are considered to interact when the distance between them is 4.0 Å or less.

In some embodiments, the interacting amino acid residue(s) in the ISVD and in human PD-1 protein are identified by X-ray crystallography by:
Mixing human PD-1 protein, and the ISVD in PBS1× buffer;
Concentrating the protein complex to 8 mg/ml;
Crystallizing by hanging drop vapor diffusion using 100 mM Na Cacodylate, 300 mM Na Acetate, 10% PEG1000, pH 6.5 at 19° C.;
Collecting X-ray diffraction data;
Processing the collected data, preferably as described in Example 13.

There is provided an immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1, wherein the ISVD cross-blocks the binding to human PD-1 protein of a polypeptide with the amino acid sequence of SEQ ID Nos: 714-1871, preferably SEQ ID NO: 1486, or wherein the ISVD is cross-blocked from binding to human PD-1 protein by a polypeptide with the amino acid sequence of SEQ ID Nos: 714-1871, preferably SEQ ID NO: 1486.

In some embodiments, the ISVD competes with a polypeptide with the amino acid sequence of SEQ ID No: 714-1871, preferably SEQ ID NO: 1486, for binding human PD-1 protein, as measured in a competition assay, such as a FACS competition assay.

Specific examples of ISVDs specifically binding to PD-1 are ISVDs that comprises 4 framework regions (FR1 to FR4, respectively) and three complementarity determining regions (CDR1 to CDR3, respectively), wherein:
CDR1 (AbM definition) has an amino acid sequence selected from:
   a) the amino acid sequence of X₁X₂X₃X₄X₅X₆X₇X₈X₉X₁₀ (SEQ ID NO: 4691); wherein X₁ is selected from G, K, F, A, Y, T, V, H, R, S, P, I, E, L, or D, X₂ is selected from D, Y, R, E, T, L, K, H, V, S, P, Q, I, G, A, or F, X₃ is selected from T, I, A, E, H, G, P, F, L, K, Q, V, R, Y, or S, X₄ is selected from F, K, D, L, T, I, A, E, V, G, H, Y, Q, S, or R, X₅ is selected from S, I, E, A, F, R, V, Y, L, T, Q, K, D, G, or H, X₆ is selected from E, Q, V, T, S, F, K, P, I, G, R, L, H, or A, X₇ is selected from N, R, Q, V, E, Y, H, A, K, L, P, G, F, I, S, or D, X₈ is selected from D, P, A, T, I, R, V, G, Y, L, F, K, S, I, H, or E, X, is selected from I, R, H, M, G, Y, Q, E, K, F, T, S, A, D, or P, and X₁₀ is selected from G, A, L, S, R, H, V, P, Q, E, I, D, Y, T, or K;
   b) amino acid sequences that have at least 80% amino acid identity with the amino acid sequence of X₁X₂X₃X₄X₅X₆X₇X₈X₉X₁₀ (SEQ ID NO: 4691); wherein X₁ is selected from G, K, F, A, Y, T, V, H, R, S, P, I, E, L, or D, X₂ is selected from D, Y, R, E, T, L, K, H, V, S, P, Q, I, G, A, or F, X₃ is selected from T, I, A, E, H, G, P, F, L, K, Q, V, R, Y, or S, X₄ is selected from F, K, D, L, T, I, A, E, V, G, H, Y, Q, S, or R, X₅ is selected from S, I, E, A, F, R, V, Y, L, T, Q, K, D, G, or H, X₆ is selected from I E, Q, V, T, S, F, K, P, I, G, R, L, H, or A, X₇ is selected from N, R, Q, V, E, Y, H, A, K, L, P, G, F, I, S, or D, X₈ is selected from D, P, A, T, I, R, V, G, Y, L, F, K, S, I, H, or E, X₉ is selected from I, R, H, M, G, Y, Q, E, K, F, T, S, A, D, or P, and X₁₀ is selected from G, A, L, S, R, H, V, P, Q, E, I, D, Y, T, or K;
   c) amino acid sequences that have 3, 2, or 1 amino acid difference with the amino acid sequences of X₁X₂X₃X₄X₅X₆X₇X₈X₉X₁₀ (SEQ ID NO: 4691); wherein X₁ is selected from G, K, F, A, Y, T, V, H, R, S, P, I, E, L, or D, X₂ is selected from D, Y, R, E, T, L, K, H, V, S, P, Q, I, G, A, or F, X₃ is selected from T, I, A, E, H, G, P, F, L, K, Q, V, R, Y, or S, X₄ is selected from F, K, D, L, T, I, A, E, V, G, H, Y, Q, S, or R, X₅ is selected from S, I, E, A, F, R, V, Y, L, T, Q, K, D, G, or H, X₆ is selected from I E, Q, V, T, S, F, K, P, I, G, R, L, H, or A, X₇ is selected from N, R, Q, V, E, Y, H, A, K, L, P, G, F, I, S, E, or D, X₈ is selected from D, P, A, T, I, R, V, G, Y, L, F, K, S, I, H, or E, X₉ is selected from I, R, H, M, G, Y, Q, E, K, F, T, S, A, D, or P, and X₁₀ is selected from G, A, L, S, R, H, V, P, Q, E, I, D, Y, T, or K;
CDR2 (AbM definition) has an amino acid sequence selected from:
   a) the amino acid sequence of X₁X₂X₃X₄X₅X₆X₇X₈X₉X₁₀ (SEQ ID NO: 4692); wherein X₁ is selected from A, G, P, F, V, I, R, Q, H, T, S, Y, K, or L, X₂ is selected from I, K, G, L, P, F, A, V, T, E, Y, S, Q, D, or R, X₃ is selected from V, K, Y, F, G, Q, A, T, S, P, I, R, D, or L, X₄ is selected from W, P, Y, Q, G, T, D, H, R, K, S, L, I, V, F, or A, X₅ is selected from P, K, Y, F, G, I, L, T, Q, H, E, D, A, S, or V, X₆ is selected from G, Q, E, P, L, F, K, I, S, A, D, R, H, or V, X₇ is selected from G, A, H, F, L, Y, T, P, Q, K, I, R, D, V, or S, X₈ is selected from E, T, I, R, H, Q, D, K, S, P, A, F, Y, V, G, or L, X₉ is selected from T, E, L, Y, F, P, Q, I, R, S, D, K, A, V, or H, and X₁₀ is selected from Y, G, I, L, E, D, H, K, A, F, S, R, P, T, Q;
   b) amino acid sequences that have at least 80% amino acid identity with the amino acid sequence of X₁X₂X₃X₄X₅X₆X₇X₈X₉X₁₀ (SEQ ID NO: 4692); wherein X₁ is selected from A, G, P, F, V, I, R, Q, H, T, S, Y, K, or L, X₂ is selected from I, K, G, L, P, F, A, V, T, E, Y, S, Q, D, or R, X₃ is selected from V, K, Y, F, G, Q, A, T, S, P, I, R, D, or L, X₄ is selected from W, P, Y, Q, G, T, D, H, R, K, S, L, I, V, F, or A, X₅ is selected from P, K, Y, F, G, I, L, T, Q, H, E, D, A, S, or V, X₆ is selected from G, Q, E, P, L, F, K, I, S, A, D, R, H, or V, X₇ is selected from G, A, H, F, L, Y, T, P, Q, K, I, R, D, V, or S, X₈ is selected from E, T, I, R, H, Q, D, K, S, P, A, F, Y, V, G, or L, X, is selected from T, E, L, Y, F, P, Q, I, R, S, D, K, A, V, or H, and X₁₀ is selected from Y, G, I, L, E, D, H, K, A, F, S, R, P, T, Q;
   c) amino acid sequences that have 3, 2, or 1 amino acid difference with the amino acid sequence of X₁X₂X₃X₄X₅X₆X₇X₈X₉X₁₀ (SEQ ID NO: 4692); wherein X₁ is selected from A, G, P, F, V, I, R, Q, H, T, S, Y, K, or L, X₂ is selected from I, K, G, L, P, F, A, V, T, E, Y, S, Q, D, or R, X₃ is selected from V, K, Y, F, G, Q, A, T, S, P, I, R, D, or L, X₄ is selected from W, P, Y, Q, G, T, D, H, R, K, S, L, I, V, F, or A, X₅ is selected from P, K, Y, F, G, I, L, T, Q, H, E, D, A, S, or V, X₆ is selected from G, Q, E, P, L, F, K, I, S, A, D, R, H, or V, X₇ is selected from G, A, H, F, L, Y, T, P, Q, K, I, R, D, V, or S, X₈ is selected from E, T, I, R, H, Q, D, K, S, P, A, F, Y, V, G, or L, X₉ is selected from T, E, L, Y, F, P, Q, I, R, S, D, K, A, V, or H, and X₁₀ is selected from Y, G, I, L, E, D, H, K, A, F, S, R, P, T, Q;
CDR3 (AbM definition) has an amino acid sequence selected from:
   a) the amino acid sequence of X₁X₂X₃X₄X₅X₆X₇X₂X₉X₁₀X₁₁X₁₂X₁₃X₁₄X₁₅X₁₆X₁₇ (SEQ ID NO: 4693); wherein X₁ is selected from K, R, L, Y, T, I, Q, A, F, P, D, H, V, S, or E, X₂ is selected from I, H, T, E, Q, S, L, K, D, G, Y, V, F, R, A, or P, X₃ is selected from F, E, K, A, R, Q, H, T, D, V, L, G, I, Y, or S, X₄ is selected from S, D, G, F, L, K, I, P, Y, V, A, or T, X₅ is selected from K, T, R, P, Y, S, E, I, L, H, D, F, V, A, or G, X₆ is selected from T, K, A, F, S, G, D, P, H, I, Q, Y, L, or V, X₇ is selected from G, F, HY, I, L, T, E, P, R, S, Q, D, K, or A, X₈ is selected from T, E, G, V, S, Q, L, H, R, P, A, F, K, I, or Y, X₉ is selected from Y, I, A, Q, G, E, F, D, K, L, S, T, R, or V, X₁₀ is selected from S, T, G, L, Q, A, I, K, E, H, D, V, R, P, F, or Y, X₁₁ is selected from A, H, Y, V, R, F, G, L, D, E, T, I, K, P, or S, X₁₂ is selected from S, T, I, D, H, V, R, P, L, K, G, Y, V, E, F, or A, X₁₃ is selected from W, Q, F, H, D, S, K, I, T, E, G, R, L, Y, or A, X₁₄ is selected from E, R, P, I, T, A, F, D, S, H, V, K, L, G, Y, or Q, X₁₅ is selected from Y, Q, L, G, V, P, K, E, I, R, H, D, or F, X₁₆ is selected from D, L, E, F, P, R, T, I, S, Y, G, A, V, K, or H, and X₁₇ is selected from F, S, T, Y, K, R, A, H, G, I, V, D, Q, P, E, or L;
   b) amino acid sequences that have at least 80% amino acid identity with the amino acid sequence of X₁X₂X₃X₄X₅X₆X₇X₈X₉X₁₀X₁₁X₁₂X₁₃X₁₄X₁₅X₁₆X₁₇ (SEQ ID NO: 4693); wherein X₁ is selected from K, R, L, Y, T, I, Q, A, F, P, D, H, V, S, or E, X₂ is selected from I, H, T, E, Q, S, L, K, D, G, Y, V, F, R, A, or P, X₃ is selected from F, E, K, A, R, Q, H, T, D, V, L, G, I, Y, or S, X₄ is selected from S, D, G, F, L, K, I, P, Y, V, A, or T, X₅ is selected from K, T, R, P, Y, S, E, I, L, H, D, F, V, A, or G, X₆ is selected from T, K, A, F, S, G, D, P, H, I, Q, Y, L, or V, X₇ is selected from G, F, H Y, I, L, T, E, P, R, S, Q, D, K, or A, X₈ is selected from T, E, G, V, S, Q, L, H, R, P, A, F, K, I, or Y, X, is selected from Y, I, A, Q, G, E, F, D, K, L, S, T, R, or V, X₁₀ is selected from S, T, G, L, Q, A, I, K, E, H, D, V, R, P, F, or Y, X₁₁ is selected from A, H, Y, V, R, F, G, L, D, E, T, I, K, P, or S, X₁₂ is selected from S, T, I, D, H, V, R, P, L, K, G, Y, V, E, F, or A, X₁₃ is selected from W, Q, F, H, D, S, K, I, T, E, G, R, L, Y, or A, X₁₄ is selected from E, R, P, I, T, A, F, D, S, H, V, K, L, G, Y, or Q, X₁₅ is selected from Y, Q, L, G, V, P, K, E, I, R, H, D, or F, X₁₆ is selected from D, L, E, F, P, R, T, I, S, Y, G, A, V, K, or H, and X₁₇ is selected from F, S, T, Y, K, R, A, H, G, I, V, D, Q, P, E, or L;
   c) amino acid sequences that have 3, 2, or 1 amino acid difference with the amino acid sequence of X₁X₂X₃X₄X₅X₆X₇X₈X₉X₁₀X₁₁X₁₂X₁₃X₁₄X₁₅X₁₆X₁₇ (SEQ ID NO: 4693); wherein X₁ is selected from K, R, L, Y, T, I, Q, A, F, P, D, H, V, S, or E, X₂ is selected from I, H, T, E, Q, S, L, K, D, G, Y, V, F, R, A, or P, X₃ is selected from F, E, K, A, R, Q, H, T, D, V, L, G, I, Y, or S, X₄ is selected from S, D, G, F, L, K, I, P, Y, V, A, or T, X₅ is selected from K, T, R, P, Y, S, E, I, L, H, D, F, V, A, or G, X₆ is selected from T, K, A, F, S, G, D, P, H, I, Q, Y, L, or V, X₇ is selected from G, F, H Y, I, L, T, E, P, R, S, Q, D, K, or A, X₈ is selected from T, E, G, V, S, Q, L, H, R, P, A, F, K, I, or Y, X₉ is selected from Y, I, A, Q, G, E, F, D, K, L, S, T, R, or V, X₁₀ is selected from S, T, G, L, Q, A, I, K, E, H, D, V, R, P, F, or Y, X₁₁ is selected from A, H, Y, V, R, F, G, L, D, E, T, I, K, P, or S, X₁₂ is selected from S, T, I, D, H, V, R, P, L, K, G, Y, V, E, F, or A, X₁₃ is selected from W, Q, F, H, D, S, K, I, T, E, G, R, L, Y, or A, X₁₄ is selected from E, R, P, I, T, A, F, D, S, H, V, K, L, G, Y, or Q, X₁₅ is selected from Y, Q, L, G, V, P, K, E, I, R, H, D, or F, X₁₆ is selected from D, L, E, F, P, R, T, I, S, Y, G, A, V, K, or H, and X₁₇ is selected from F, S, T, Y, K, R, A, H, G, I, V, D, Q, P, E, or L.

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 4691, 4692 and 4693.

In one embodiment, the ISVD comprises CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 714-1871.

In one embodiment, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 1957;
   b) amino acid sequences that have at least 80% amino acid identity with the amino acid sequence of SEQ ID NO: 1957; and
   c) amino acid sequences that have 3, 2, or 1 amino acid difference with the amino acid sequences of SEQ ID NO: 1957;
and
CDR2 (AbM definition) has an amino acid sequence selected from:
   a) the amino acid sequence of SEQ ID NO: 2127;
   b) amino acid sequences that have at least 80% amino acid identity with the amino acid sequence of SEQ ID NO: 2127;
   c) amino acid sequences that have 3, 2, or 1 amino acid difference with the amino acid sequence of SEQ ID NO: 2127;
and
CDR3 (AbM definition) has an amino acid sequence selected from:
   a) the amino acid sequence of SEQ ID NOs: 2337;
   b) amino acid sequences that have at least 80% amino acid identity with the amino acid sequence of SEQ ID NO: 2337;
   c) amino acid sequences that have 3, 2, or 1 amino acid difference with the amino acid sequence of SEQ ID NO: 2337.

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NOs: 1957, 2127, and 2337.

In one embodiment, the ISVD comprises CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 793, 1505, 1092, 1085, 1078, 1486, and 1083.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2096, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2063, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2117, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2117, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2062, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2062, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2097, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2064, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2064, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2061, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2061, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2097, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2096, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2063, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2064, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2127, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2061, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2061, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2096, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2096, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2063, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2117, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2117, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2062, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2062, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2097, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2063, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2097, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2064, and a CDR3 (AbM numbering) that is the amino acid sequence of SEQ ID NO: 2338.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2339.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2340.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2341.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2342.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2343.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2344.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2364.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2365.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2366.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2367.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2368.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2369.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2370.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2371.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2372.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2373.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2374.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2375.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2376.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2377.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2378.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2379.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2380.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2381.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2382.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2383.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2384.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2385.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2386.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2387.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2388.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 1890.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2390.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4044.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3917.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4042.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4002.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3999.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3982.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3833.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4009.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4007.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4005.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3929.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4003.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3813.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3835.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3834.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3847.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3920.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3919.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4000.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4035.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4017.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4021.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4013.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4041.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4032.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4027.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4024.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4036.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3935.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4015.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4019.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4038.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4030.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4039.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4011.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4022.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3900.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3921.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4025.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4031.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3819.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3824.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4045.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3817.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3905.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3818.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4028.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4046.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3918.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3822.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3821.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4047.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3820.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2352.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2096, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2061, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2061, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2096, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2063, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2063, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2171, and a CDR3 (AbM numbering) that is the amino acid sequence of SEQ ID NO: 3940.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2058, and a CDR3 (AbM numbering) that is the amino acid sequence of SEQ ID NO: 2359.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2062, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2184, and a CDR3 (AbM numbering) that is the amino acid sequence of SEQ ID NO: 3940.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2171, and a CDR3 (AbM numbering) that is the amino acid sequence of SEQ ID NO: 2359.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2184, and a CDR3 (AbM numbering) that is the amino acid sequence of SEQ ID NO: 2359.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2062, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2117, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2058, and a CDR3 (AbM numbering) that is the amino acid sequence of SEQ ID NO: 3940.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2064, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2127, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2097, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2117, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2064, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2097, and a CDR3 (AbM numbering) that is the amino acid sequence of SEQ ID NO: 3812.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2171, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2058, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2184, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2003, a CDR2 (AbM numbering) that is the amino acid sequence of SEQ ID NO: 2183, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3841.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4001.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3998.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3842.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3844.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2189, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1954, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1961, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1965, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1964, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1958, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2172, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1967, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1962, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1963, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1956, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1959, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1955, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1968, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1960, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2182, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2194, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2186, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2054, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2173, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2174, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2191, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2190, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2185, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2055, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2193, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2192, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2195, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2187, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2091, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2053, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2050, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1984, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1947, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1987, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1981, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1985, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1988, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1979, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1989, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1973, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1983, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1992, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1991, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1994, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1940, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1953, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2180, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1978, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1970, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1982, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1952, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1976, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1972, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1950, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1969, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1951, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1975, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1974, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1948, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1949, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1971, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1986, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1932, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2169, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2059, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2144, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2086, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2087, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2089, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2092, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2167, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2165, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2088, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2077, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2170, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2085, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2090, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2084, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2161, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2083, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1995, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2094, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1943, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2160, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2159, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2095, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2158, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2157, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2166, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2078, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2163, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2093, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2162, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2156, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2164, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2168, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2072, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2081, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2065, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2056, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2068, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2067, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2179, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2070, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2052, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2181, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2177, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2178, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2036, a CDR2 (AbM numbering) that is the amino acid sequence of SEQ ID NO: 2175, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2051, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1938, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2073, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1980, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2176, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2079, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2075, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2080, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2076, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2074, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2082, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2071, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2069, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2060, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2066, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2057, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1977, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1945, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2039, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2032, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1908, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2023, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2034, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2029, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2028, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2026, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1905, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2046, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2044, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2045, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2037, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2042, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2043, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2041, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2038, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2035, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1907, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2033, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2040, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1906, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2030, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1927, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2007, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2006, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1925, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1928, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1936, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1931, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2013, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 3883, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2009, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1934, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2008, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1935, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1933, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1926, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1929, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1930, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2005, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1996, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2000, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1946, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1990, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1942, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1941, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1944, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1993, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1939, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2002, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1999, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1997, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2004, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2001, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1998, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2031, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1937, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1915, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1910, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1911, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1914, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1913, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1918, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2027, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2025, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1909, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2036, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2024, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1912, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1917, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1916, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1919, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2021, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1923, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2014, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2018, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2012, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1922, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1924, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2019, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2010, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1920, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2011, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2020, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2015, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2017, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2016, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 2022, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1921, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2148, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2155, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4034.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3868.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3964.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3869.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4006.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4014.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3865.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3867.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4012.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4004.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2361.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2363.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2362.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3932.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2389.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4008.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3963.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4016.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4020.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3934.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4023.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3816.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4018.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 1893.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4010.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4037.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4026.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4029.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4048.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4040.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3968.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4043.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2357.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3975.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2354.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3871.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3879.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3878.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3876.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3875.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2353.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2356.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3977.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3976.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2355.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3866.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3930.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3969.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3967.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3971.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3965.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2360.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3933.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2358.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3873.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3882.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3880.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2359.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3966.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3881.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3874.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3970.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3855.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3864.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3856.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 4033.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3850.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 1891.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3990.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3994.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3996.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3892.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3992.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3993.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3988.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3991.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3987.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3851.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3958.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3997.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3950.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3995.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2153, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2149, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3840.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3849.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3848.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3989.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3845.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3838.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3839.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3846.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3843.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3836.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3852.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3837.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3948.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3949.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3953.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3941.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3858.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3863.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3938.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3859.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3861.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3853.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3946.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3862.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3860.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3937.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3857.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3936.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3940.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3956.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3973.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3944.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3960.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3943.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3942.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3961.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3962.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3959.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3945.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3954.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3939.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3951.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3952.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 1892.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3854.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3974.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2351.

In one embodiment, the ISVD comprises a CDR1 (AbM numbering) that is the amino acid sequence of SEQ ID NO: 1966, a CDR2 (AbM numbering) that is the amino acid sequence of SEQ ID NO: 2127, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2119, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2121, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2115, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2114, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2118, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2120, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2103, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2108, and a CDR3 (AbM numbering) that is the amino acid sequence of SEQ ID NO: 3813.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2129, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2104, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2109, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2110, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2113, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2106, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2116, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2125, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2124, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2111, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2125, and a CDR3 (AbM numbering) that is the amino acid sequence of SEQ ID NO: 3937.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2122, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2126, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2123, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2141, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2112, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2133, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2139, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2132, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2138, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2099, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2134, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2150, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2145, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2147, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2146, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2151, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2154, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2152, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2102, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2140, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2136, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2098, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2107, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2108, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2101, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2128, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2137, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2105, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2130, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2143, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2142, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2131, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2100, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2135, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3981.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3978.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2188, and a CDR3 (AbM numbering) that is the amino acid sequence of SEQ ID NO: 2337.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3986.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3984.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2350.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3980.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2345.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2348.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3979.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2346.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2349.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3985.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3983.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 2347.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127, and a CDR3 (AbM numbering) that is the amino acid sequence of SEQ ID NO: 3877.

In some embodiments, the ISVD additionally comprises D1 and/or V2 (Kabat numbering).

In some embodiments, the ISVD:
i) has 80% amino acid sequence identity with one of the amino acid sequences of SEQ ID NOs: 714-1871, in which for the purposes of determining the degree of amino acid identity, the amino acid residues that form the CDR sequences are disregarded;
and in which:
ii) preferably one or more of the amino acid residues at positions 11, 37, 44, 45, 47, 83, 84, 103, 104 and 108 according to the Kabat numbering are chosen from the Hallmark residues mentioned in Table 1.

In some embodiments, the ISVD essentially consists of a heavy chain variable domain sequence that is derived from a conventional four-chain antibody or that essentially consist of a heavy chain variable domain sequence that is derived from heavy chain antibody.

In some embodiments, the ISVD essentially consists of a VHH, a humanized VHH, a camelized VH, a domain antibody, a single domain antibody, or a dAb.

In some embodiments, the ISVD is a humanized ISVD that is chosen from SEQ ID NOs: 714-792, 794-1871 or from the group consisting of amino acid sequences that have more than 80%, preferably more than 90%, more preferably more than 95%, such as 99% or more amino acid sequence identity with at least one of the amino acid sequences of SEQ ID NOs: 714-1871.

In some embodiments, the ISVD is a humanized ISVD that is chosen from SEQ ID NO: 1505, 1092, 1085, 1078, 1486, or 1083 or from the group consisting of amino acid sequences that have more than 80%, preferably more than 90%, more preferably more than 95%, such as 99% or more amino acid sequence identity with at least one of the amino acid sequences of SEQ ID NOs: 793, 1505, 1092, 1085, 1078, 1786, or 1083.

In some embodiments, the amino acid sequence is chosen from the group consisting of SEQ ID NO: 793 and SEQ ID NOs: 714-792, 794-1871.

In some embodiments, the amino acid sequence is chosen from the group consisting of SEQ ID NO: 793 and SEQ ID NOs: 1505, 1092, 1085, 1078, 1486, or 1083.

In some embodiments, the ISVD specifically binds to human PD-1 with a dissociation constant (K_{D}) of 10⁻⁹ to 10⁻¹² moles/litre or less, and preferably 5×10⁻¹⁰ to 10⁻¹² moles/litre or less and more preferably 5×10⁻¹⁰ to 1×10⁻¹¹ moles/litre, such as 4.0×10⁻¹⁰ moles/litre, as determined by Surface Plasmon Resonance, e.g., at 25° C.

In some embodiments, the ISVD specifically binds to human PD-1 with a kₒₙ-rate of between 10⁴ M⁻¹s⁻¹ to about 10⁷ M⁻¹ s⁻¹, preferably between 10⁵ M⁻¹ s⁻¹ and 10⁷ M⁻¹ s⁻¹, more preferably between 10⁵ M⁻¹ s 1 and 10⁶ M⁻¹ s⁻¹, such as between 10⁵ M⁻¹ s⁻¹ and 5×10⁵ M⁻¹ s⁻¹, such as 3.7×10⁵ M⁻¹ s⁻¹, as determined by Surface Plasmon Resonance, e.g., at 25° C.

In some embodiments, the ISVD specifically binds to human PD-1 with a k_{off} rate between 10⁻³ s⁻¹ (t1/2=0.69 s) and 10⁻⁷ s⁻¹ (providing a near irreversible complex with a t1/2 of multiple days), preferably between 10⁻³ s⁻¹ and 5×10⁶ s⁻¹, more preferably between 5×10⁻⁴ s⁻¹ and 5×10⁶ s⁻¹, such as between 5×10⁻⁴ s⁻¹ and 10⁻⁵ s⁻¹, such as 1.5×10⁻⁴ s⁻¹, ¹, as determined by Surface Plasmon Resonance, e.g., at 25° C.

In some embodiments, the ISVD specifically binds to human and cyno PD-1 and does not bind to CD28, CTLA-4, ICOS, and BTLA or other members of the CD28 receptor family.

In some embodiments, the ISVD antagonizes an activity of PD-L1.

In some embodiments, the ISVD blocks the interaction of human PD-1 with human PD-L1 with a potency (IC50 value) of 5×10⁻⁸ M or lower, more preferably of 10⁻⁸ M or lower, or even of 10⁻⁹ M or lower, such as between 10⁻¹¹ M and 10⁻⁸ M, between 10⁻¹⁰ M and 10⁻⁹ M, between 10⁻¹⁰ M and 10⁻⁸ M or between 10⁻⁹ M and 10⁻⁸ M, such as 6.07×10⁻⁹ M, for example, as measured in a FACS competition assay on CHO cells expressing human PD-1 with hPD-L1 as a competitor.

In some embodiments, the ISVD blocks the interaction of cyno PD-1 with cyno PD-L1 with a potency (IC50 value) of 10⁻⁷ M or lower, more preferably of 5×10⁻⁸ M or lower, or even of 10⁻⁸ M or lower, such as between 10⁻¹¹ M and 10⁻⁷ M, between 10⁻¹⁰ M and 10⁻⁹ M, between 10⁻¹⁰ M and 5×10⁻⁸ M or between 10⁻⁸ M and 10⁻⁷ M, such as 1.19×10⁻⁸ M, for example, as measured in a FACS competition assay on CHO cells expressing cyno PD-1 with hPD-L1 as a competitor.

In some embodiments, the ISVD blocks the binding of hPD-1 to hPD-L1 by at least 50%, such as at least 60%, 70%, 80%, 90%, 95%, 99% or even more, as determined by ligand competition, AlphaScreen, or competitive binding assays (such as competition ELISA or competition FACS).

In some embodiments, the ISVD induces T-cell activation with a potency (EC50 value) of 10⁻⁷ M or lower, more preferably of 5×10⁻⁸ M or lower, or even of 10⁻⁸ M or lower, such as between 10⁻¹¹ M and 10⁻⁷ M, such as between 10⁻¹⁰ M and 10⁻⁸ M, between 10⁻¹⁰ M and 10⁻⁷ M or between 10⁻⁸ M and 10⁻⁷ M, such as 4.32×10⁻⁸ M, 3.24×10⁻⁸ M or 7.1×10⁻⁸ M, as determined in a TT assay.

Specific examples of such ISVDs that specifically bind to PD-1 have one or more, or all, framework regions as indicated for A0294041A11 and sequence optimized variants thereof in Table A-3 and Table A-8 (in addition to the CDRs as defined above). In one embodiment, the ISVD comprises or consists of the full amino acid sequence of A029400053 (SEQ ID NO: 714), A029404160 (SEQ ID NO: 715), A029404142 (SEQ ID NO: 716), A029404141 (SEQ ID NO: 717), A029404140 (SEQ ID NO: 718), A029404139 (SEQ ID NO: 719), A029404138 (SEQ ID NO: 720), A029404091 (SEQ ID NO: 721), A029404137 (SEQ ID NO: 722), A029404135 (SEQ ID NO: 723), A029404134 (SEQ ID NO: 724), A029404133 (SEQ ID NO: 725), A029404132 (SEQ ID NO: 726), A029404131 (SEQ ID NO: 727), A029404130 (SEQ ID NO: 728), A029404136 (SEQ ID NO: 729), A029404129 (SEQ ID NO: 730), A029404128 (SEQ ID NO: 731), A029404147 (SEQ ID NO: 732), A029404145 (SEQ ID NO: 733), A029404144 (SEQ ID NO: 734), A029404159 (SEQ ID NO: 735), A029404158 (SEQ ID NO: 736), A029404157 (SEQ ID NO: 737), A029404156 (SEQ ID NO: 738), A029404146 (SEQ ID NO: 739), A029404155 (SEQ ID NO: 740), A029404127 (SEQ ID NO: 741), A029404143 (SEQ ID NO: 742), A029404151 (SEQ ID NO: 743), A029404150 (SEQ ID NO: 744), A029404149 (SEQ ID NO: 745), A029404148 (SEQ ID NO: 746), A029404154 (SEQ ID NO: 747), A029404092 (SEQ ID NO: 748), A029404153 (SEQ ID NO: 749), A029404117 (SEQ ID NO: 750), A029404124 (SEQ ID NO: 751), A029404106 (SEQ ID NO: 752), A029404105 (SEQ ID NO: 753), A029404104 (SEQ ID NO: 754), A029404103 (SEQ ID NO: 755), A029404102 (SEQ ID NO: 756), A029404116 (SEQ ID NO: 757), A029404101 (SEQ ID NO: 758), A029404099 (SEQ ID NO: 759), A029404098 (SEQ ID NO: 760), A029404097 (SEQ ID NO: 761), A029404096 (SEQ ID NO: 762), A029404095 (SEQ ID NO: 763), A029404094 (SEQ ID NO: 764), A029404100 (SEQ ID NO: 765), A029404152 (SEQ ID NO: 766), A029404126 (SEQ ID NO: 767), A029404111 (SEQ ID NO: 768), A029404109 (SEQ ID NO: 769), A029404108 (SEQ ID NO: 770), A029404123 (SEQ ID NO: 771), A029404122 (SEQ ID NO: 772), A029404121 (SEQ ID NO: 773), A029404120 (SEQ ID NO: 774), A029404110 (SEQ ID NO: 775), A029404119 (SEQ ID NO: 776), A029404125 (SEQ ID NO: 777), A029404107 (SEQ ID NO: 778), A029404115 (SEQ ID NO: 779), A029404114 (SEQ ID NO: 780), A029404113 (SEQ ID NO: 781), A029404112 (SEQ ID NO: 782), A029404118 (SEQ ID NO: 783), A029404093 (SEQ ID NO: 784), A029404161 (SEQ ID NO: 785), A029404169 (SEQ ID NO: 786), A029404199 (SEQ ID NO: 787), A029404213 (SEQ ID NO: 788), A029404212 (SEQ ID NO: 789), A029404211 (SEQ ID NO: 790), A029404210 (SEQ ID NO: 791), A029404209 (SEQ ID NO: 792), A0294041A11 (SEQ ID NO: 793), A029404208 (SEQ ID NO: 794), A029404206 (SEQ ID NO: 795), A029404205 (SEQ ID NO: 796), A029404204 (SEQ ID NO: 797), A029404203 (SEQ ID NO: 798), A029404202 (SEQ ID NO: 799), A029404201 (SEQ ID NO: 800), A029404207 (SEQ ID NO: 801), g_A029404200 (SEQ ID NO: 802), A029404214 (SEQ ID NO: 803), A029404224 (SEQ ID NO: 804), A029404230 (SEQ ID NO: 805), A029404229 (SEQ ID NO: 806), A029404228 (SEQ ID NO: 807), A029404227 (SEQ ID NO: 808), A029404226 (SEQ ID NO: 809), A029404225 (SEQ ID NO: 810), A029404223 (SEQ ID NO: 811), A029404216 (SEQ ID NO: 812), A029404222 (SEQ ID NO: 813), A029404221 (SEQ ID NO: 814), A029404220 (SEQ ID NO: 815), A029404219 (SEQ ID NO: 816), A029404218 (SEQ ID NO: 817), A029404217 (SEQ ID NO: 818), A029404215 (SEQ ID NO: 819), A029404170 (SEQ ID NO: 820), A029404163 (SEQ ID NO: 821), A029404196 (SEQ ID NO: 822), A029404178 (SEQ ID NO: 823), A029404176 (SEQ ID NO: 824), A029404175 (SEQ ID NO: 825), A029404174 (SEQ ID NO: 826), A029404173 (SEQ ID NO: 827), A029404172 (SEQ ID NO: 828), A029404171 (SEQ ID NO: 829), A029404177 (SEQ ID NO: 830), A029404198 (SEQ ID NO: 831), A029404168 (SEQ ID NO: 832), A029404167 (SEQ ID NO: 833), A029404166 (SEQ ID NO: 834), A029404165 (SEQ ID NO: 835), A029404164 (SEQ ID NO: 836), A029404197 (SEQ ID NO: 837), A029404162 (SEQ ID NO: 838), A029404179 (SEQ ID NO: 839), A029404187 (SEQ ID NO: 840), A029404180 (SEQ ID NO: 841), A029404194 (SEQ ID NO: 842), A029404193 (SEQ ID NO: 843), A029404192 (SEQ ID NO: 844), A029404191 (SEQ ID NO: 845), A029404190 (SEQ ID NO: 846), A029404189 (SEQ ID NO: 847), A029404195 (SEQ ID NO: 848), A029404188 (SEQ ID NO: 849), A029404186 (SEQ ID NO: 850), A029404185 (SEQ ID NO: 851), A029404184 (SEQ ID NO: 852), A029404183 (SEQ ID NO: 853), A029404182 (SEQ ID NO: 854), A029404181 (SEQ ID NO: 855), A029404090 (SEQ ID NO: 856), A029404232 (SEQ ID NO: 857), A029403119 (SEQ ID NO: 858), A029403601 (SEQ ID NO: 859), A029403599 (SEQ ID NO: 860), A029403598 (SEQ ID NO: 861), A029403593 (SEQ ID NO: 862), A029403590 (SEQ ID NO: 863), A029403580 (SEQ ID NO: 864), A029403650 (SEQ ID NO: 865), A029403563 (SEQ ID NO: 866), A029403576 (SEQ ID NO: 867), A029403571 (SEQ ID NO: 868), A029403570 (SEQ ID NO: 869), A029403568 (SEQ ID NO: 870), A029403566 (SEQ ID NO: 871), A029403565 (SEQ ID NO: 872), A029403564 (SEQ ID NO: 873), A029403572 (SEQ ID NO: 874), A029403602 (SEQ ID NO: 875), A029403632 (SEQ ID NO: 876), A029403603 (SEQ ID NO: 877), A029403641 (SEQ ID NO: 878), A029403640 (SEQ ID NO: 879), A029403638 (SEQ ID NO: 880), A029403637 (SEQ ID NO: 881), A029403636 (SEQ ID NO: 882), A029403634 (SEQ ID NO: 883), A029403648 (SEQ ID NO: 884), A029403633 (SEQ ID NO: 885), A029403621 (SEQ ID NO: 886), A029403619 (SEQ ID NO: 887), A029403615 (SEQ ID NO: 888), A029403610 (SEQ ID NO: 889), A029403609 (SEQ ID NO: 890), A029403606 (SEQ ID NO: 891), A029404068 (SEQ ID NO: 892), A029403654 (SEQ ID NO: 893), A029403660 (SEQ ID NO: 894), A029403512 (SEQ ID NO: 895), A029403493 (SEQ ID NO: 896), A029403492 (SEQ ID NO: 897), A029403490 (SEQ ID NO: 898), A029403489 (SEQ ID NO: 899), A029403487 (SEQ ID NO: 900), A029403486 (SEQ ID NO: 901), A029403495 (SEQ ID NO: 902), A029403559 (SEQ ID NO: 903), A029403482 (SEQ ID NO: 904), A029403481 (SEQ ID NO: 905), A029403480 (SEQ ID NO: 906), A029403479 (SEQ ID NO: 907), A029403475 (SEQ ID NO: 908), A029403561 (SEQ ID NO: 909), A029403651 (SEQ ID NO: 910), A029403514 (SEQ ID NO: 911), A029403525 (SEQ ID NO: 912), A029403518 (SEQ ID NO: 913), A029403547 (SEQ ID NO: 914), A029403538 (SEQ ID NO: 915), A029403535 (SEQ ID NO: 916), A029403533 (SEQ ID NO: 917), A029403532 (SEQ ID NO: 918), A029403530 (SEQ ID NO: 919), A029403558 (SEQ ID NO: 920), A029403529 (SEQ ID NO: 921), A029403524 (SEQ ID NO: 922), A029403523 (SEQ ID NO: 923), A029403522 (SEQ ID NO: 924), A029403521 (SEQ ID NO: 925), A029403520 (SEQ ID NO: 926), A029403519 (SEQ ID NO: 927), A029403715 (SEQ ID NO: 928), A029404070 (SEQ ID NO: 929), A029403749 (SEQ ID NO: 930), A029404087 (SEQ ID NO: 931), A029404063 (SEQ ID NO: 932), A029404062 (SEQ ID NO: 933), A029404067 (SEQ ID NO: 934), A029404066 (SEQ ID NO: 935), A029404065 (SEQ ID NO: 936), A029404079 (SEQ ID NO: 937), A029404064 (SEQ ID NO: 938), A029404060 (SEQ ID NO: 939), A029404055 (SEQ ID NO: 940), A029404054 (SEQ ID NO: 941), A029404059 (SEQ ID NO: 942), A029404058 (SEQ ID NO: 943), A029404057 (SEQ ID NO: 944), A029404072 (SEQ ID NO: 945), A029404056 (SEQ ID NO: 946), A029403600 (SEQ ID NO: 947), A029404080 (SEQ ID NO: 948), A029403578 (SEQ ID NO: 949), A029404086 (SEQ ID NO: 950), A029404085 (SEQ ID NO: 951), A029404083 (SEQ ID NO: 952), A029404082 (SEQ ID NO: 953), A029404081 (SEQ ID NO: 954), A029404061 (SEQ ID NO: 955), A029404053 (SEQ ID NO: 956), A029404078 (SEQ ID NO: 957), A029404077 (SEQ ID NO: 958), A029404076 (SEQ ID NO: 959), A029404075 (SEQ ID NO: 960), A029404074 (SEQ ID NO: 961), A029404073 (SEQ ID NO: 962), A029404071 (SEQ ID NO: 963), A029404069 (SEQ ID NO: 964), A029404019 (SEQ ID NO: 965), A029404018 (SEQ ID NO: 966), A029403728 (SEQ ID NO: 967), A029403678 (SEQ ID NO: 968), A029403674 (SEQ ID NO: 969), A029403673 (SEQ ID NO: 970), A029403701 (SEQ ID NO: 971), A029403686 (SEQ ID NO: 972), A029403679 (SEQ ID NO: 973), A029403672 (SEQ ID NO: 974), A029403778 (SEQ ID NO: 975), A029403667 (SEQ ID NO: 976), A029403666 (SEQ ID NO: 977), A029403662 (SEQ ID NO: 978), A029403671 (SEQ ID NO: 979), A029403669 (SEQ ID NO: 980), A029403668 (SEQ ID NO: 981), A029404231 (SEQ ID NO: 982), A029403771 (SEQ ID NO: 983), A029404089 (SEQ ID NO: 984), A029403670 (SEQ ID NO: 985), A029403770 (SEQ ID NO: 986), A029403777 (SEQ ID NO: 987), A029403776 (SEQ ID NO: 988), A029403775 (SEQ ID NO: 989), A029403774 (SEQ ID NO: 990), A029403773 (SEQ ID NO: 991), A029403772 (SEQ ID NO: 992), A029403779 (SEQ ID NO: 993), A029403690 (SEQ ID NO: 994), A029403769 (SEQ ID NO: 995), A029403768 (SEQ ID NO: 996), A029403767 (SEQ ID NO: 997), A029403766 (SEQ ID NO: 998), A029403765 (SEQ ID NO: 999), A029404088 (SEQ ID NO: 1000), A029403120 (SEQ ID NO: 1001), A029404233 (SEQ ID NO: 1002), A029404615 (SEQ ID NO: 1003), A029404614 (SEQ ID NO: 1004), A029404613 (SEQ ID NO: 1005), A029404612 (SEQ ID NO: 1006), A029404611 (SEQ ID NO: 1007), A029404617 (SEQ ID NO: 1008), A029404610 (SEQ ID NO: 1009), A029404608 (SEQ ID NO: 1010), A029404421 (SEQ ID NO: 1011), A029404420 (SEQ ID NO: 1012), A029404419 (SEQ ID NO: 1013), A029404418 (SEQ ID NO: 1014), A029404417 (SEQ ID NO: 1015), A029404609 (SEQ ID NO: 1016), A029404416 (SEQ ID NO: 1017), A029404700 (SEQ ID NO: 1018), A029404693 (SEQ ID NO: 1019), A029404413 (SEQ ID NO: 1020), A029404698 (SEQ ID NO: 1021), A029404697 (SEQ ID NO: 1022), A029404696 (SEQ ID NO: 1023), A029404695 (SEQ ID NO: 1024), A029404694 (SEQ ID NO: 1025), A029404619 (SEQ ID NO: 1026), A029404618 (SEQ ID NO: 1027), A029404415 (SEQ ID NO: 1028), A029404690 (SEQ ID NO: 1029), A029404689 (SEQ ID NO: 1030), A029404688 (SEQ ID NO: 1031), A029404687 (SEQ ID NO: 1032), A029404686 (SEQ ID NO: 1033), A029404692 (SEQ ID NO: 1034), A029404620 (SEQ ID NO: 1035), A029404699 (SEQ ID NO: 1036), A029404691 (SEQ ID NO: 1037), A029404406 (SEQ ID NO: 1038), A029404404 (SEQ ID NO: 1039), A029404392 (SEQ ID NO: 1040), A029404391 (SEQ ID NO: 1041), A029404390 (SEQ ID NO: 1042), A029404389 (SEQ ID NO: 1043), A029404395 (SEQ ID NO: 1044), A029404388 (SEQ ID NO: 1045), A029404386 (SEQ ID NO: 1046), A029404385 (SEQ ID NO: 1047), A029404384 (SEQ ID NO: 1048), A029404383 (SEQ ID NO: 1049), A029404382 (SEQ ID NO: 1050), A029404381 (SEQ ID NO: 1051), A029404387 (SEQ ID NO: 1052), A029404414 (SEQ ID NO: 1053), A029404701 (SEQ ID NO: 1054), A029404702 (SEQ ID NO: 1055), A029404393 (SEQ ID NO: 1056), A029404411 (SEQ ID NO: 1057), A029404410 (SEQ ID NO: 1058), A029404409 (SEQ ID NO: 1059), A029404408 (SEQ ID NO: 1060), A029404407 (SEQ ID NO: 1061), A029404397 (SEQ ID NO: 1062), A029404396 (SEQ ID NO: 1063), A029404394 (SEQ ID NO: 1064), A029404403 (SEQ ID NO: 1065), A029404402 (SEQ ID NO: 1066), A029404401 (SEQ ID NO: 1067), A029404400 (SEQ ID NO: 1068), A029404399 (SEQ ID NO: 1069), A029404405 (SEQ ID NO: 1070), A029404398 (SEQ ID NO: 1071), A029404412 (SEQ ID NO: 1072), A029404703 (SEQ ID NO: 1073), A029405257 (SEQ ID NO: 1074), A029405250 (SEQ ID NO: 1075), A029405258 (SEQ ID NO: 1076), A029405256 (SEQ ID NO: 1077), A029405255 (SEQ ID NO: 1078), A029405254 (SEQ ID NO: 1079), A029405253 (SEQ ID NO: 1080), A029405252 (SEQ ID NO: 1081), A029405261 (SEQ ID NO: 1082), A029405259 (SEQ ID NO: 1083), A029405249 (SEQ ID NO: 1084), A029405247 (SEQ ID NO: 1085), A029405246 (SEQ ID NO: 1086), A029405244 (SEQ ID NO: 1087), A029405243 (SEQ ID NO: 1088), A029405242 (SEQ ID NO: 1089), A029405241 (SEQ ID NO: 1090), A029405248 (SEQ ID NO: 1091), A029405240 (SEQ ID NO: 1092), A029405239 (SEQ ID NO: 1093), A029405273 (SEQ ID NO: 1094), A029405272 (SEQ ID NO: 1095), A029405271 (SEQ ID NO: 1096), A029405270 (SEQ ID NO: 1097), A029405260 (SEQ ID NO: 1098), A029405276 (SEQ ID NO: 1099), A029405275 (SEQ ID NO: 1100), A029405269 (SEQ ID NO: 1101), A029405265 (SEQ ID NO: 1102), A029405264 (SEQ ID NO: 1103), A029405263 (SEQ ID NO: 1104), A029405262 (SEQ ID NO: 1105), A029405268 (SEQ ID NO: 1106), A029405267 (SEQ ID NO: 1107), A029405266 (SEQ ID NO: 1108), A029405238 (SEQ ID NO: 1109), A029404758 (SEQ ID NO: 1110), A029404756 (SEQ ID NO: 1111), A029404754 (SEQ ID NO: 1112), A029404753 (SEQ ID NO: 1113), A029404759 (SEQ ID NO: 1114), A029404752 (SEQ ID NO: 1115), A029404709 (SEQ ID NO: 1116), A029404708 (SEQ ID NO: 1117), A029404707 (SEQ ID NO: 1118), A029404706 (SEQ ID NO: 1119), A029404705 (SEQ ID NO: 1120), A029404704 (SEQ ID NO: 1121), A029404751 (SEQ ID NO: 1122), A029404760 (SEQ ID NO: 1123), A029404761 (SEQ ID NO: 1124), A029404757 (SEQ ID NO: 1125), A029404762 (SEQ ID NO: 1126), A029404755 (SEQ ID NO: 1127), A029405090 (SEQ ID NO: 1128), A029405089 (SEQ ID NO: 1129), A029405237 (SEQ ID NO: 1130), A029405088 (SEQ ID NO: 1131), A029405087 (SEQ ID NO: 1132), A029405086 (SEQ ID NO: 1133), A029405085 (SEQ ID NO: 1134), A029405084 (SEQ ID NO: 1135), 29405082 (SEQ ID NO: 1136), A029405037 (SEQ ID NO: 1137), A029405083 (SEQ ID NO: 1138), A029404784 (SEQ ID NO: 1139), A029404778 (SEQ ID NO: 1140), A029404773 (SEQ ID NO: 1141), A029404768 (SEQ ID NO: 1142), A029404380 (SEQ ID NO: 1143), A029403474 (SEQ ID NO: 1144), A029403485 (SEQ ID NO: 1145), A029403484 (SEQ ID NO: 1146), A029404284 (SEQ ID NO: 1147), A029404283 (SEQ ID NO: 1148), A029404282 (SEQ ID NO: 1149), A029404281 (SEQ ID NO: 1150), A029404280 (SEQ ID NO: 1151), A029404279 (SEQ ID NO: 1152), A029404285 (SEQ ID NO: 1153), A029404278 (SEQ ID NO: 1154), A029404276 (SEQ ID NO: 1155), A029404275 (SEQ ID NO: 1156), A029404274 (SEQ ID NO: 1157), A029404273 (SEQ ID NO: 1158), A029404272 (SEQ ID NO: 1159), A029404271 (SEQ ID NO: 1160), A029404277 (SEQ ID NO: 1161), A029404270 (SEQ ID NO: 1162), A029404303 (SEQ ID NO: 1163), A029404296 (SEQ ID NO: 1164), A029404267 (SEQ ID NO: 1165), A029404301 (SEQ ID NO: 1166), A029404300 (SEQ ID NO: 1167), A029404299 (SEQ ID NO: 1168), A029404298 (SEQ ID NO: 1169), A029404297 (SEQ ID NO: 1170), A029404287 (SEQ ID NO: 1171), A029404286 (SEQ ID NO: 1172), A029404269 (SEQ ID NO: 1173), A029404293 (SEQ ID NO: 1174), A029404292 (SEQ ID NO: 1175), A029404291 (SEQ ID NO: 1176), A029404290 (SEQ ID NO: 1177), A029404289 (SEQ ID NO: 1178), A029404295 (SEQ ID NO: 1179), A029404288 (SEQ ID NO: 1180), A029404302 (SEQ ID NO: 1181), A029404294 (SEQ ID NO: 1182), A029404260 (SEQ ID NO: 1183), A029404258 (SEQ ID NO: 1184), A029404246 (SEQ ID NO: 1185), A029404245 (SEQ ID NO: 1186), A029404244 (SEQ ID NO: 1187), A029404243 (SEQ ID NO: 1188), A029404249 (SEQ ID NO: 1189), A029404242 (SEQ ID NO: 1190), A029404240 (SEQ ID NO: 1191), A029404239 (SEQ ID NO: 1192), A029404238 (SEQ ID NO: 1193), A029404237 (SEQ ID NO: 1194), A029404236 (SEQ ID NO: 1195), A029404235 (SEQ ID NO: 1196), A029404241 (SEQ ID NO: 1197), A029404268 (SEQ ID NO: 1198), A029404304 (SEQ ID NO: 1199), A029404305 (SEQ ID NO: 1200), A029404247 (SEQ ID NO: 1201), A029404265 (SEQ ID NO: 1202), A029404264 (SEQ ID NO: 1203), A029404263 (SEQ ID NO: 1204), A029404262 (SEQ ID NO: 1205), A029404261 (SEQ ID NO: 1206), A029404251 (SEQ ID NO: 1207), A029404250 (SEQ ID NO: 1208), A029404248 (SEQ ID NO: 1209), A029404257 (SEQ ID NO: 1210), A029404256 (SEQ ID NO: 1211), A029404255 (SEQ ID NO: 1212), A029404254 (SEQ ID NO: 1213), A029404253 (SEQ ID NO: 1214), A029404259 (SEQ ID NO: 1215), A029404252 (SEQ ID NO: 1216), A029404266 (SEQ ID NO: 1217), A029404306 (SEQ ID NO: 1218), A029404357 (SEQ ID NO: 1219), A029404351 (SEQ ID NO: 1220), A029404358 (SEQ ID NO: 1221), A029404356 (SEQ ID NO: 1222), A029404355 (SEQ ID NO: 1223), A029404354 (SEQ ID NO: 1224), A029404353 (SEQ ID NO: 1225), A029404352 (SEQ ID NO: 1226), A029404361 (SEQ ID NO: 1227), A029404359 (SEQ ID NO: 1228), A029404350 (SEQ ID NO: 1229), A029404348 (SEQ ID NO: 1230), A029404347 (SEQ ID NO: 1231), A029404346 (SEQ ID NO: 1232), A029404345 (SEQ ID NO: 1233), A029404344 (SEQ ID NO: 1234), A029404343 (SEQ ID NO: 1235), A029404349 (SEQ ID NO: 1236), A029404342 (SEQ ID NO: 1237), A029404341 (SEQ ID NO: 1238), A029404374 (SEQ ID NO: 1239), A029404373 (SEQ ID NO: 1240), A029404372 (SEQ ID NO: 1241), A029404371 (SEQ ID NO: 1242), A029404360 (SEQ ID NO: 1243), A029404376 (SEQ ID NO: 1244), A029404375 (SEQ ID NO: 1245), A029404370 (SEQ ID NO: 1246), A029404366 (SEQ ID NO: 1247), A029404365 (SEQ ID NO: 1248), A029404363 (SEQ ID NO: 1249), A029404362 (SEQ ID NO: 1250), A029404369 (SEQ ID NO: 1251), A029404368 (SEQ ID NO: 1252), A029404367 (SEQ ID NO: 1253), A029404340 (SEQ ID NO: 1254), A029404320 (SEQ ID NO: 1255), A029404318 (SEQ ID NO: 1256), A029404316 (SEQ ID NO: 1257), A029404315 (SEQ ID NO: 1258), A029404321 (SEQ ID NO: 1259), A029404314 (SEQ ID NO: 1260), A029404312 (SEQ ID NO: 1261), A029404311 (SEQ ID NO: 1262), A029404310 (SEQ ID NO: 1263), A029404309 (SEQ ID NO: 1264), A029404308 (SEQ ID NO: 1265), A029404307 (SEQ ID NO: 1266), A029404313 (SEQ ID NO: 1267), A029404322 (SEQ ID NO: 1268), A029404323 (SEQ ID NO: 1269), A029404319 (SEQ ID NO: 1270), A029404324 (SEQ ID NO: 1271), A029404317 (SEQ ID NO: 1272), A029404338 (SEQ ID NO: 1273), A029404337 (SEQ ID NO: 1274), A029404339 (SEQ ID NO: 1275), A029404336 (SEQ ID NO: 1276), A029404335 (SEQ ID NO: 1277), A029404334 (SEQ ID NO: 1278), A029404333 (SEQ ID NO: 1279), A029404332 (SEQ ID NO: 1280), A029404330 (SEQ ID NO: 1281), A029404329 (SEQ ID NO: 1282), A029404331 (SEQ ID NO: 1283), A029404328 (SEQ ID NO: 1284), A029404327 (SEQ ID NO: 1285), A029404326 (SEQ ID NO: 1286), A029404325 (SEQ ID NO: 1287), A029404378 (SEQ ID NO: 1288), A029404234 (SEQ ID NO: 1289), A029404616 (SEQ ID NO: 1290), A029403471 (SEQ ID NO: 1291), A029403467 (SEQ ID NO: 1292), A029403463 (SEQ ID NO: 1293), A029402531 (SEQ ID NO: 1294), A029402528 (SEQ ID NO: 1295), A029402527 (SEQ ID NO: 1296), A029402514 (SEQ ID NO: 1297), A029402511 (SEQ ID NO: 1298), A029402506 (SEQ ID NO: 1299), A029402574 (SEQ ID NO: 1300), A029402491 (SEQ ID NO: 1301), A029402504 (SEQ ID NO: 1302), A029402499 (SEQ ID NO: 1303), A029402496 (SEQ ID NO: 1304), A029402495 (SEQ ID NO: 1305), A029402494 (SEQ ID NO: 1306), A029402493 (SEQ ID NO: 1307), A029402492 (SEQ ID NO: 1308), A029402503 (SEQ ID NO: 1309), A029402532 (SEQ ID NO: 1310), A029402546 (SEQ ID NO: 1311), A029402533 (SEQ ID NO: 1312), A029402572 (SEQ ID NO: 1313), A029402571 (SEQ ID NO: 1314), A029402570 (SEQ ID NO: 1315), A029402553 (SEQ ID NO: 1316), A029402552 (SEQ ID NO: 1317), A029402550 (SEQ ID NO: 1318), A029402573 (SEQ ID NO: 1319), A029402548 (SEQ ID NO: 1320), A029402542 (SEQ ID NO: 1321), A029402540 (SEQ ID NO: 1322), A029402538 (SEQ ID NO: 1323), A029402537 (SEQ ID NO: 1324), A029402535 (SEQ ID NO: 1325), A029402534 (SEQ ID NO: 1326), A029402709 (SEQ ID NO: 1327), A029402576 (SEQ ID NO: 1328), A029402577 (SEQ ID NO: 1329), A029402436 (SEQ ID NO: 1330), A029402432 (SEQ ID NO: 1331), A029402430 (SEQ ID NO: 1332), A029402429 (SEQ ID NO: 1333), A029402428 (SEQ ID NO: 1334), A029402427 (SEQ ID NO: 1335), A029402424 (SEQ ID NO: 1336), A029402434 (SEQ ID NO: 1337), A029402486 (SEQ ID NO: 1338), A029402419 (SEQ ID NO: 1339), A029402406 (SEQ ID NO: 1340), A029402404 (SEQ ID NO: 1341), A029402399 (SEQ ID NO: 1342), A029402395 (SEQ ID NO: 1343), A029402487 (SEQ ID NO: 1344), A029402575 (SEQ ID NO: 1345), A029402437 (SEQ ID NO: 1346), A029402463 (SEQ ID NO: 1347), A029402447 (SEQ ID NO: 1348), A029402474 (SEQ ID NO: 1349), A029402472 (SEQ ID NO: 1350), A029402471 (SEQ ID NO: 1351), A029402470 (SEQ ID NO: 1352), A029402467 (SEQ ID NO: 1353), A029402465 (SEQ ID NO: 1354), A029402477 (SEQ ID NO: 1355), A029402464 (SEQ ID NO: 1356), A029402462 (SEQ ID NO: 1357), A029402458 (SEQ ID NO: 1358), A029402457 (SEQ ID NO: 1359), A029402456 (SEQ ID NO: 1360), A029402449 (SEQ ID NO: 1361), A029402448 (SEQ ID NO: 1362), A029402619 (SEQ ID NO: 1363), A029402715 (SEQ ID NO: 1364), A029402623 (SEQ ID NO: 1365), A029402764 (SEQ ID NO: 1366), A029402698 (SEQ ID NO: 1367), A029402696 (SEQ ID NO: 1368), A029402706 (SEQ ID NO: 1369), A029402704 (SEQ ID NO: 1370), A029402700 (SEQ ID NO: 1371), A029402736 (SEQ ID NO: 1372), A029402699 (SEQ ID NO: 1373), A029402694 (SEQ ID NO: 1374), A029402686 (SEQ ID NO: 1375), A029402678 (SEQ ID NO: 1376), A029402693 (SEQ ID NO: 1377), A029402692 (SEQ ID NO: 1378), A029402690 (SEQ ID NO: 1379), A029402726 (SEQ ID NO: 1380), A029402689 (SEQ ID NO: 1381), A029402392 (SEQ ID NO: 1382), A029402737 (SEQ ID NO: 1383), A029402393 (SEQ ID NO: 1384), A029402758 (SEQ ID NO: 1385), A029402753 (SEQ ID NO: 1386), A029402752 (SEQ ID NO: 1387), A029402749 (SEQ ID NO: 1388), A029402738 (SEQ ID NO: 1389), A029402695 (SEQ ID NO: 1390), A029402674 (SEQ ID NO: 1391), A029402735 (SEQ ID NO: 1392), A029402734 (SEQ ID NO: 1393), A029402731 (SEQ ID NO: 1394), A029402729 (SEQ ID NO: 1395), A029402728 (SEQ ID NO: 1396), A029402727 (SEQ ID NO: 1397), A029402725 (SEQ ID NO: 1398), A029402712 (SEQ ID NO: 1399), A029402672 (SEQ ID NO: 1400), A029402670 (SEQ ID NO: 1401), A029402622 (SEQ ID NO: 1402), A029402613 (SEQ ID NO: 1403), A029402612 (SEQ ID NO: 1404), A029402608 (SEQ ID NO: 1405), A029402617 (SEQ ID NO: 1406), A029402615 (SEQ ID NO: 1407), A029402614 (SEQ ID NO: 1408), A029402603 (SEQ ID NO: 1409), A029402666 (SEQ ID NO: 1410), A029402582 (SEQ ID NO: 1411), A029402581 (SEQ ID NO: 1412), A029402579 (SEQ ID NO: 1413), A029402600 (SEQ ID NO: 1414), A029402593 (SEQ ID NO: 1415), A029402589 (SEQ ID NO: 1416), A029402394 (SEQ ID NO: 1417), A029402653 (SEQ ID NO: 1418), A029402421 (SEQ ID NO: 1419), A029402594 (SEQ ID NO: 1420), A029402652 (SEQ ID NO: 1421), A029402664 (SEQ ID NO: 1422), A029402661 (SEQ ID NO: 1423), A029402660 (SEQ ID NO: 1424), A029402659 (SEQ ID NO: 1425), A029402656 (SEQ ID NO: 1426), A029402655 (SEQ ID NO: 1427), A029402667 (SEQ ID NO: 1428), A029402616 (SEQ ID NO: 1429), A029402643 (SEQ ID NO: 1430), A029402635 (SEQ ID NO: 1431), A029402631 (SEQ ID NO: 1432), A029402625 (SEQ ID NO: 1433), A029402624 (SEQ ID NO: 1434), A029402420 (SEQ ID NO: 1435), A029404379 (SEQ ID NO: 1436), A029405277 (SEQ ID NO: 1437), A029402391 (SEQ ID NO: 1438), A029402148 (SEQ ID NO: 1439), A029402144 (SEQ ID NO: 1440), A029402143 (SEQ ID NO: 1441), A029402139 (SEQ ID NO: 1442), A029402157 (SEQ ID NO: 1443), A029402130 (SEQ ID NO: 1444), A029402127 (SEQ ID NO: 1445), A029402125 (SEQ ID NO: 1446), A029402122 (SEQ ID NO: 1447), A029402121 (SEQ ID NO: 1448), A029402120 (SEQ ID NO: 1449), A029402119 (SEQ ID NO: 1450), A029402129 (SEQ ID NO: 1451), A029402118 (SEQ ID NO: 1452), A029402199 (SEQ ID NO: 1453), A029402186 (SEQ ID NO: 1454), A029400195 (SEQ ID NO: 1455), A029402197 (SEQ ID NO: 1456), A029402193 (SEQ ID NO: 1457), A029402191 (SEQ ID NO: 1458), A029402190 (SEQ ID NO: 1459), A029402189 (SEQ ID NO: 1460), A029402159 (SEQ ID NO: 1461), A029402158 (SEQ ID NO: 1462), A029400197 (SEQ ID NO: 1463), A029402177 (SEQ ID NO: 1464), A029402170 (SEQ ID NO: 1465), A029402167 (SEQ ID NO: 1466), A029402162 (SEQ ID NO: 1467), A029402161 (SEQ ID NO: 1468), A029402181 (SEQ ID NO: 1469), A029402160 (SEQ ID NO: 1470), A029402198 (SEQ ID NO: 1471), A029402178 (SEQ ID NO: 1472), A029400158 (SEQ ID NO: 1473), A029400156 (SEQ ID NO: 1474), A029400144 (SEQ ID NO: 1475), A029400143 (SEQ ID NO: 1476), A029400142 (SEQ ID NO: 1477), A029400141 (SEQ ID NO: 1478), A029400147 (SEQ ID NO: 1479), A029400140 (SEQ ID NO: 1480), A029400138 (SEQ ID NO: 1481), A029400137 (SEQ ID NO: 1482), A029400136 (SEQ ID NO: 1483), A029400135 (SEQ ID NO: 1484), A029400075 (SEQ ID NO: 1485), A029400073 (SEQ ID NO: 1486), A029400139 (SEQ ID NO: 1487), A029400196 (SEQ ID NO: 1488), A029402200 (SEQ ID NO: 1489), A029402154 (SEQ ID NO: 1490), A029400145 (SEQ ID NO: 1491), A029400193 (SEQ ID NO: 1492), A029400192 (SEQ ID NO: 1493), A029400191 (SEQ ID NO: 1494), A029400160 (SEQ ID NO: 1495), A029400159 (SEQ ID NO: 1496), A029400149 (SEQ ID NO: 1497), A029400148 (SEQ ID NO: 1498), A029400146 (SEQ ID NO: 1499), A029400155 (SEQ ID NO: 1500), A029400154 (SEQ ID NO: 1501), A029400153 (SEQ ID NO: 1502), A029400152 (SEQ ID NO: 1503), A029400151 (SEQ ID NO: 1504), A029400157 (SEQ ID NO: 1505), A029400150 (SEQ ID NO: 1506), A029400194 (SEQ ID NO: 1507), A029402153 (SEQ ID NO: 1508), A029402202 (SEQ ID NO: 1509), A029402324 (SEQ ID NO: 1510), A029402349 (SEQ ID NO: 1511), A029402346 (SEQ ID NO: 1512), A029402344 (SEQ ID NO: 1513), A029402334 (SEQ ID NO: 1514), A029402332 (SEQ ID NO: 1515), A029402327 (SEQ ID NO: 1516), A029402304 (SEQ ID NO: 1517), A029402307 (SEQ ID NO: 1518), A029402319 (SEQ ID NO: 1519), A029402315 (SEQ ID NO: 1520), A029402313 (SEQ ID NO: 1521), A029402312 (SEQ ID NO: 1522), A029402311 (SEQ ID NO: 1523), A029402310 (SEQ ID NO: 1524), A029402309 (SEQ ID NO: 1525), A029402316 (SEQ ID NO: 1526), A029402350 (SEQ ID NO: 1527), A029402374 (SEQ ID NO: 1528), A029402351 (SEQ ID NO: 1529), A029402389 (SEQ ID NO: 1530), A029402388 (SEQ ID NO: 1531), A029402386 (SEQ ID NO: 1532), A029402385 (SEQ ID NO: 1533), A029402384 (SEQ ID NO: 1534), A029402383 (SEQ ID NO: 1535), A029402390 (SEQ ID NO: 1536), A029402377 (SEQ ID NO: 1537), A029402366 (SEQ ID NO: 1538), A029402365 (SEQ ID NO: 1539), A029402363 (SEQ ID NO: 1540), A029402362 (SEQ ID NO: 1541), A029402354 (SEQ ID NO: 1542), A029402352 (SEQ ID NO: 1543), A029402308 (SEQ ID NO: 1544), A029402204 (SEQ ID NO: 1545), A029402347 (SEQ ID NO: 1546), A029402236 (SEQ ID NO: 1547), A029402235 (SEQ ID NO: 1548), A029402233 (SEQ ID NO: 1549), A029402232 (SEQ ID NO: 1550), A029402248 (SEQ ID NO: 1551), A029402231 (SEQ ID NO: 1552), A029402225 (SEQ ID NO: 1553), A029402213 (SEQ ID NO: 1554), A029402210 (SEQ ID NO: 1555), A029402209 (SEQ ID NO: 1556), A029402207 (SEQ ID NO: 1557), A029402206 (SEQ ID NO: 1558), A029402230 (SEQ ID NO: 1559), A029402249 (SEQ ID NO: 1560), A029402244 (SEQ ID NO: 1561), A029402238 (SEQ ID NO: 1562), A029402252 (SEQ ID NO: 1563), A029402275 (SEQ ID NO: 1564), A029402274 (SEQ ID NO: 1565), A029402291 (SEQ ID NO: 1566), 29402289 (SEQ ID NO: 1567), A029402283 (SEQ ID NO: 1568), A029402281 (SEQ ID NO: 1569), A029402280 (SEQ ID NO: 1570), A029402276 (SEQ ID NO: 1571), A029402256 (SEQ ID NO: 1572), A029402292 (SEQ ID NO: 1573), A029402273 (SEQ ID NO: 1574), A029402272 (SEQ ID NO: 1575), A029402271 (SEQ ID NO: 1576), A029402270 (SEQ ID NO: 1577), A029402265 (SEQ ID NO: 1578), A029402259 (SEQ ID NO: 1579), A029402765 (SEQ ID NO: 1580), A029404377 (SEQ ID NO: 1581), A029402766 (SEQ ID NO: 1582), A029402773 (SEQ ID NO: 1583), A029403283 (SEQ ID NO: 1584), A029403247 (SEQ ID NO: 1585), A029403246 (SEQ ID NO: 1586), A029403245 (SEQ ID NO: 1587), A029403243 (SEQ ID NO: 1588), A029403240 (SEQ ID NO: 1589), A029403294 (SEQ ID NO: 1590), A029403239 (SEQ ID NO: 1591), A029403236 (SEQ ID NO: 1592), A029403235 (SEQ ID NO: 1593), A029403234 (SEQ ID NO: 1594), A029403232 (SEQ ID NO: 1595), A029403229 (SEQ ID NO: 1596), A029403226 (SEQ ID NO: 1597), A029403237 (SEQ ID NO: 1598), A029403213 (SEQ ID NO: 1599), A029403211 (SEQ ID NO: 1600), A029403258 (SEQ ID NO: 1601), A029403252 (SEQ ID NO: 1602), A029403249 (SEQ ID NO: 1603), A029403278 (SEQ ID NO: 1604), A029403276 (SEQ ID NO: 1605), A029403274 (SEQ ID NO: 1606), A029403273 (SEQ ID NO: 1607), A029403255 (SEQ ID NO: 1608), A029403272 (SEQ ID NO: 1609), A029403210 (SEQ ID NO: 1610), A029403248 (SEQ ID NO: 1611), A029403267 (SEQ ID NO: 1612), A029403265 (SEQ ID NO: 1613), A029403264 (SEQ ID NO: 1614), A029403263 (SEQ ID NO: 1615), A029403271 (SEQ ID NO: 1616), A029403297 (SEQ ID NO: 1617), A029403270 (SEQ ID NO: 1618), A029403176 (SEQ ID NO: 1619), A029403203 (SEQ ID NO: 1620), A029403158 (SEQ ID NO: 1621), A029403150 (SEQ ID NO: 1622), A029403148 (SEQ ID NO: 1623), A029403141 (SEQ ID NO: 1624), A029403139 (SEQ ID NO: 1625), A029403172 (SEQ ID NO: 1626), A029403134 (SEQ ID NO: 1627), A029403132 (SEQ ID NO: 1628), A029403131 (SEQ ID NO: 1629), A029403130 (SEQ ID NO: 1630), A029403129 (SEQ ID NO: 1631), A029403128 (SEQ ID NO: 1632), A029403127 (SEQ ID NO: 1633), A029403133 (SEQ ID NO: 1634), A029403268 (SEQ ID NO: 1635), A029403208 (SEQ ID NO: 1636), A029403167 (SEQ ID NO: 1637), A029403165 (SEQ ID NO: 1638), A029403162 (SEQ ID NO: 1639), A029403201 (SEQ ID NO: 1640), A029403199 (SEQ ID NO: 1641), A029403198 (SEQ ID NO: 1642), A029403188 (SEQ ID NO: 1643), A029403166 (SEQ ID NO: 1644), A029403183 (SEQ ID NO: 1645), A029403206 (SEQ ID NO: 1646), A029403159 (SEQ ID NO: 1647), A029403171 (SEQ ID NO: 1648), A029403170 (SEQ ID NO: 1649), A029403169 (SEQ ID NO: 1650), A029403168 (SEQ ID NO: 1651), A029403177 (SEQ ID NO: 1652), A029403298 (SEQ ID NO: 1653), A029403308 (SEQ ID NO: 1654), A029403306 (SEQ ID NO: 1655), A029403389 (SEQ ID NO: 1656), A029403426 (SEQ ID NO: 1657), A029403387 (SEQ ID NO: 1658), A029403424 (SEQ ID NO: 1659), A029403423 (SEQ ID NO: 1660), A029403421 (SEQ ID NO: 1661), A029403440 (SEQ ID NO: 1662), A029403420 (SEQ ID NO: 1663), A029403416 (SEQ ID NO: 1664), A029403415 (SEQ ID NO: 1665), A029403414 (SEQ ID NO: 1666), A029403413 (SEQ ID NO: 1667), A029403412 (SEQ ID NO: 1668), A029403406 (SEQ ID NO: 1669), A029403417 (SEQ ID NO: 1670), A029403394 (SEQ ID NO: 1671), A029403444 (SEQ ID NO: 1672), A029403445 (SEQ ID NO: 1673), A029402768 (SEQ ID NO: 1674), A029402767 (SEQ ID NO: 1675), A029403461 (SEQ ID NO: 1676), A029403460 (SEQ ID NO: 1677), A029403459 (SEQ ID NO: 1678), A029403458 (SEQ ID NO: 1679), A029403427 (SEQ ID NO: 1680), A029403457 (SEQ ID NO: 1681), A029403456 (SEQ ID NO: 1682), A029403455 (SEQ ID NO: 1683), A029403453 (SEQ ID NO: 1684), A029403452 (SEQ ID NO: 1685), A029403451 (SEQ ID NO: 1686), A029403446 (SEQ ID NO: 1687), A029403454 (SEQ ID NO: 1688), A029403391 (SEQ ID NO: 1689), A029403388 (SEQ ID NO: 1690), A029403383 (SEQ ID NO: 1691), A029403336 (SEQ ID NO: 1692), A029403328 (SEQ ID NO: 1693), A029403323 (SEQ ID NO: 1694), A029403316 (SEQ ID NO: 1695), A029403314 (SEQ ID NO: 1696), A029403310 (SEQ ID NO: 1697), A029403309 (SEQ ID NO: 1698), A029403335 (SEQ ID NO: 1699), A029403339 (SEQ ID NO: 1700), A029403305 (SEQ ID NO: 1701), A029403304 (SEQ ID NO: 1702), A029403303 (SEQ ID NO: 1703), A029403300 (SEQ ID NO: 1704), A029403299 (SEQ ID NO: 1705), A029403338 (SEQ ID NO: 1706), A029403379 (SEQ ID NO: 1707), A029403380 (SEQ ID NO: 1708), A029403345 (SEQ ID NO: 1709), A029403381 (SEQ ID NO: 1710), A029403360 (SEQ ID NO: 1711), A029403354 (SEQ ID NO: 1712), A029403378 (SEQ ID NO: 1713), A029403377 (SEQ ID NO: 1714), A029403375 (SEQ ID NO: 1715), A029403374 (SEQ ID NO: 1716), A029403364 (SEQ ID NO: 1717), A029403353 (SEQ ID NO: 1718), A029403344 (SEQ ID NO: 1719), A029403342 (SEQ ID NO: 1720), A029403352 (SEQ ID NO: 1721), A029403351 (SEQ ID NO: 1722), A029403349 (SEQ ID NO: 1723), A029403347 (SEQ ID NO: 1724), A029403126 (SEQ ID NO: 1725), A029402769 (SEQ ID NO: 1726), A029403123 (SEQ ID NO: 1727), A029402505 (SEQ ID NO: 1728), A029402920 (SEQ ID NO: 1729), A029402884 (SEQ ID NO: 1730), A029402883 (SEQ ID NO: 1731), A029402882 (SEQ ID NO: 1732), A029402880 (SEQ ID NO: 1733), A029402879 (SEQ ID NO: 1734), A029402921 (SEQ ID NO: 1735), A029402878 (SEQ ID NO: 1736), A029402876 (SEQ ID NO: 1737), A029402868 (SEQ ID NO: 1738), A029402864 (SEQ ID NO: 1739), A029402861 (SEQ ID NO: 1740), A029402857 (SEQ ID NO: 1741), A029402856 (SEQ ID NO: 1742), A029402877 (SEQ ID NO: 1743), A029402853 (SEQ ID NO: 1744), A029402850 (SEQ ID NO: 1745), A029402890 (SEQ ID NO: 1746), A029402888 (SEQ ID NO: 1747), A029402886 (SEQ ID NO: 1748), A029402912 (SEQ ID NO: 1749), A029402909 (SEQ ID NO: 1750), A029402908 (SEQ ID NO: 1751), A029402907 (SEQ ID NO: 1752), A029402889 (SEQ ID NO: 1753), A029402906 (SEQ ID NO: 1754), A029402848 (SEQ ID NO: 1755), A029402885 (SEQ ID NO: 1756), A029402900 (SEQ ID NO: 1757), A029402895 (SEQ ID NO: 1758), A029402894 (SEQ ID NO: 1759), A029402892 (SEQ ID NO: 1760), A029402905 (SEQ ID NO: 1761), A029402926 (SEQ ID NO: 1762), A029402904 (SEQ ID NO: 1763), A029402827 (SEQ ID NO: 1764), A029402844 (SEQ ID NO: 1765), A029402806 (SEQ ID NO: 1766), A029402805 (SEQ ID NO: 1767), A029402804 (SEQ ID NO: 1768), A029402803 (SEQ ID NO: 1769), A029402802 (SEQ ID NO: 1770), A029402824 (SEQ ID NO: 1771), A029402798 (SEQ ID NO: 1772), A029402789 (SEQ ID NO: 1773), A029402786 (SEQ ID NO: 1774), A029402782 (SEQ ID NO: 1775), A029402779 (SEQ ID NO: 1776), A029402775 (SEQ ID NO: 1777), A029402774 (SEQ ID NO: 1778), A029402797 (SEQ ID NO: 1779), A029402902 (SEQ ID NO: 1780), A029402847 (SEQ ID NO: 1781), A029402813 (SEQ ID NO: 1782), A029402811 (SEQ ID NO: 1783), A029402810 (SEQ ID NO: 1784), A029402843 (SEQ ID NO: 1785), A029402842 (SEQ ID NO: 1786), A029402841 (SEQ ID NO: 1787), A029402840 (SEQ ID NO: 1788), A029402812 (SEQ ID NO: 1789), A029402831 (SEQ ID NO: 1790), A029402846 (SEQ ID NO: 1791), A029402808 (SEQ ID NO: 1792), A029402818 (SEQ ID NO: 1793), A029402817 (SEQ ID NO: 1794), A029402816 (SEQ ID NO: 1795), A029402815 (SEQ ID NO: 1796), A029402829 (SEQ ID NO: 1797), A029402928 (SEQ ID NO: 1798), A029402939 (SEQ ID NO: 1799), A029402938 (SEQ ID NO: 1800), A029403026 (SEQ ID NO: 1801), A029403070 (SEQ ID NO: 1802), A029403023 (SEQ ID NO: 1803), A029403064 (SEQ ID NO: 1804), A029403062 (SEQ ID NO: 1805), A029403060 (SEQ ID NO: 1806), A029403073 (SEQ ID NO: 1807), A029403057 (SEQ ID NO: 1808), A029403052 (SEQ ID NO: 1809), A029403051 (SEQ ID NO: 1810), A029403050 (SEQ ID NO: 1811), A029403048 (SEQ ID NO: 1812), A029403046 (SEQ ID NO: 1813), A029403041 (SEQ ID NO: 1814), A029403054 (SEQ ID NO: 1815), A029403033 (SEQ ID NO: 1816), A029403078 (SEQ ID NO: 1817), A029403080 (SEQ ID NO: 1818), A029402530 (SEQ ID NO: 1819), A029403107 (SEQ ID NO: 1820), A029403106 (SEQ ID NO: 1821), A029403104 (SEQ ID NO: 1822), A029403099 (SEQ ID NO: 1823), A029403096 (SEQ ID NO: 1824), A029403072 (SEQ ID NO: 1825), A029403093 (SEQ ID NO: 1826), A029403088 (SEQ ID NO: 1827), A029403087 (SEQ ID NO: 1828), A029403084 (SEQ ID NO: 1829), A029403083 (SEQ ID NO: 1830), A029403082 (SEQ ID NO: 1831), A029403081 (SEQ ID NO: 1832), A029403086 (SEQ ID NO: 1833), A029403028 (SEQ ID NO: 1834), A029403025 (SEQ ID NO: 1835), A029403020 (SEQ ID NO: 1836), A029402965 (SEQ ID NO: 1837), A029402955 (SEQ ID NO: 1838), A029402954 (SEQ ID NO: 1839), A029402949 (SEQ ID NO: 1840), A029402946 (SEQ ID NO: 1841), A029402943 (SEQ ID NO: 1842), A029402941 (SEQ ID NO: 1843), A029402962 (SEQ ID NO: 1844), A029402971 (SEQ ID NO: 1845), A029402937 (SEQ ID NO: 1846), A029402936 (SEQ ID NO: 1847), A029402935 (SEQ ID NO: 1848), A029402934 (SEQ ID NO: 1849), A029402933 (SEQ ID NO: 1850), A029402970 (SEQ ID NO: 1851), A029403014 (SEQ ID NO: 1852), A029403015 (SEQ ID NO: 1853), A029402977 (SEQ ID NO: 1854), A029403016 (SEQ ID NO: 1855), A029402996 (SEQ ID NO: 1856), A029402991 (SEQ ID NO: 1857), A029403013 (SEQ ID NO: 1858), A029403012 (SEQ ID NO: 1859), A029403011 (SEQ ID NO: 1860), A029403010 (SEQ ID NO: 1861), A029403009 (SEQ ID NO: 1862), A029402988 (SEQ ID NO: 1863), A029402975 (SEQ ID NO: 1864), A029402972 (SEQ ID NO: 1865), A029402986 (SEQ ID NO: 1866), A029402985 (SEQ ID NO: 1867), A029402983 (SEQ ID NO: 1868), A029402979 (SEQ ID NO: 1869), A029403121 (SEQ ID NO: 1870), A029405278 (SEQ ID NO: 1871).

In another embodiment, the ISVD specifically binding to human PD-1 may have a sequence identity of more than 90%, such as more than 95% or even more than 99%, with SEQ ID NO: 714, wherein the CDRs are as defined above. In one embodiment, the ISVD specifically binding to PD-1 comprises or consists of the amino acid sequence of SEQ ID NO: 714.

In another embodiment, the ISVD specifically binding to human PD-1 may have a sequence identity of more than 90%, such as more than 95% or even more than 99%, with SEQ ID NO: 715, wherein the CDRs are as defined above. In one embodiment, the ISVD specifically binding to PD-1 comprises or consists of the amino acid sequence of SEQ ID NO: 715.

In another embodiment, the ISVD specifically binding to human PD-1 may have a sequence identity of more than 90%, such as more than 95% or even more than 99%, with SEQ ID NO: 716, wherein the CDRs are as defined above. In one embodiment, the ISVD specifically binding to PD-1 comprises or consists of the amino acid sequence of SEQ ID NO: 716.

In another embodiment, the ISVD specifically binding to human PD-1 may have a sequence identity of more than 90%, such as more than 95% or even more than 99%, with SEQ ID NO: 717, wherein the CDRs are as defined above. In one embodiment, the ISVD specifically binding to PD-1 comprises or consists of the amino acid sequence of SEQ ID NO: 717.

In another embodiment, the ISVD specifically binding to human PD-1 may have a sequence identity of more than 90%, such as more than 95% or even more than 99%, with SEQ ID NO: 718, wherein the CDRs are as defined above. In one embodiment, the ISVD specifically binding to PD-1 comprises or consists of the amino acid sequence of SEQ ID NO: 718.

In another embodiment, the ISVD specifically binding to human PD-1 may have a sequence identity of more than 90%, such as more than 95% or even more than 99%, with SEQ ID NO: 719, wherein the CDRs are as defined above. In one embodiment, the ISVD specifically binding to PD-1 comprises or consists of the amino acid sequence of SEQ ID NO: 719.

In another embodiment, the ISVD specifically binding to human PD-1 may have a sequence identity of more than 90%, such as more than 95% or even more than 99%, with SEQ ID NO: 720, wherein the CDRs are as defined above. In one embodiment, the ISVD specifically binding to PD-1 comprises or consists of the amino acid sequence of SEQ ID NO: 720.

In another embodiment, the ISVD specifically binding to human PD-1 may have a sequence identity of more than 90%, such as more than 95% or even more than 99%, with SEQ ID NO: 721, wherein the CDRs are as defined above. In one embodiment, the ISVD specifically binding to PD-1 comprises or consists of the amino acid sequence of SEQ ID NO: 721.

In another embodiment, the ISVD specifically binding to human PD-1 may have a sequence identity of more than 90%, such as more than 95% or even more than 99%, with SEQ ID NO: 722, wherein the CDRs are as defined above. In one embodiment, the ISVD specifically binding to PD-1 comprises or consists of the amino acid sequence of SEQ ID NO: 722.

In another embodiment, the ISVD specifically binding to human PD-1 may have a sequence identity of more than 90%, such as more than 95% or even more than 99%, with SEQ ID NO: 723, wherein the CDRs are as defined above. In one embodiment, the ISVD specifically binding to PD-1 comprises or consists of the amino acid sequence of SEQ ID NO: 723.

In another embodiment, the ISVD specifically binding to human PD-1 may have a sequence identity of more than 90%, such as more than 95% or even more than 99%, with SEQ ID NO: 724, wherein the CDRs are as defined above. In one embodiment, the ISVD specifically binding to PD-1 comprises or consists of the amino acid sequence of SEQ ID NO: 724.

In another embodiment, the ISVD specifically binding to human PD-1 may have a sequence identity of more than 90%, such as more than 95% or even more than 99%, with SEQ ID NO: 725, wherein the CDRs are as defined above. In one embodiment, the ISVD specifically binding to PD-1 comprises or consists of the amino acid sequence of SEQ ID NO: 725.

In another embodiment, the ISVD specifically binding to human PD-1 may have a sequence identity of more than 90%, such as more than 95% or even more than 99%, with SEQ ID NO: 726, wherein the CDRs are as defined above. In one embodiment, the ISVD specifically binding to PD-1 comprises or consists of the amino acid sequence of SEQ ID NO: 726.

In another embodiment, the ISVD specifically binding to human PD-1 may have a sequence identity of more than 90%, such as more than 95% or even more than 99%, with SEQ ID NO: 727, wherein the CDRs are as defined above. In one embodiment, the ISVD specifically binding to PD-1 comprises or consists of the amino acid sequence of SEQ ID NO: 727.

In another embodiment, the ISVD specifically binding to human PD-1 may have a sequence identity of more than 90%, such as more than 95% or even more than 99%, with SEQ ID NO: 728, wherein the CDRs are as defined above. In one embodiment, the ISVD specifically binding to PD-1 comprises or consists of the amino acid sequence of SEQ ID NO: 728.

In another embodiment, the ISVD specifically binding to human PD-1 may have a sequence identity of more than 90%, such as more than 95% or even more than 99%, with SEQ ID NO: 729, wherein the CDRs are as defined above. In one embodiment, the ISVD specifically binding to PD-1 comprises or consists of the amino acid sequence of SEQ ID NO: 729.

In another embodiment, the ISVD specifically binding to human PD-1 may have a sequence identity of more than 90%, such as more than 95% or even more than 99%, with SEQ ID NO: 730, wherein the CDRs are as defined above. In one embodiment, the ISVD specifically binding to PD-1 comprises or consists of the amino acid sequence of SEQ ID NO: 730.

In another embodiment, the ISVD specifically binding to human PD-1 may have a sequence identity of more than 90%, such as more than 95% or even more than 99%, with SEQ ID NO: 731, wherein the CDRs are as defined above. In one embodiment, the ISVD specifically binding to PD-1 comprises or consists of the amino acid sequence of SEQ ID NO: 731.

In another embodiment, the ISVD specifically binding to human PD-1 may have a sequence identity of more than 90%, such as more than 95% or even more than 99%, with SEQ ID NO: 732, wherein the CDRs are as defined above. In one embodiment, the ISVD specifically binding to PD-1 comprises or consists of the amino acid sequence of SEQ ID NO: 732.

In another embodiment, the ISVD specifically binding to human PD-1 may have a sequence identity of more than 90%, such as more than 95% or even more than 99%, with SEQ ID NO: 733, wherein the CDRs are as defined above. In one embodiment, the ISVD specifically binding to PD-1 comprises or consists of the amino acid sequence of SEQ ID NO: 733.

In another embodiment, the ISVD specifically binding to human PD-1 may have a sequence identity of more than 90%, such as more than 95% or even more than 99%, with SEQ ID NO: 734, wherein the CDRs are as defined above. In one embodiment, the ISVD specifically binding to PD-1 comprises or consists of the amino acid sequence of SEQ ID NO: 734.

In another embodiment, the ISVD specifically binding to human PD-1 may have a sequence identity of more than 90%, such as more than 95% or even more than 99%, with SEQ ID NO: 735, wherein the CDRs are as defined above. In one embodiment, the ISVD specifically binding to PD-1 comprises or consists of the amino acid sequence of SEQ ID NO: 735.

In another embodiment, the ISVD specifically binding to human PD-1 may have a sequence identity of more than 90%, such as more than 95% or even more than 99%, with SEQ ID NO: 736, wherein the CDRs are as defined above. In one embodiment, the ISVD specifically binding to PD-1 comprises or consists of the amino acid sequence of SEQ ID NO: 736.

In another embodiment, the ISVD specifically binding to human PD-1 may have a sequence identity of more than 90%, such as more than 95% or even more than 99%, with SEQ ID NO: 737, wherein the CDRs are as defined above. In one embodiment, the ISVD specifically binding to PD-1 comprises or consists of the amino acid sequence of SEQ ID NO: 737.

In another embodiment, the ISVD specifically binding to human PD-1 may have a sequence identity of more than 90%, such as more than 95% or even more than 99%, with SEQ ID NO: 738, wherein the CDRs are as defined above. In one embodiment, the ISVD specifically binding to PD-1 comprises or consists of the amino acid sequence of SEQ ID NO: 738.

In another embodiment, the ISVD specifically binding to human PD-1 may have a sequence identity of more than 90%, such as more than 95% or even more than 99%, with SEQ ID NO: 739, wherein the CDRs are as defined above. In one embodiment, the ISVD specifically binding to PD-1 comprises or consists of the amino acid sequence of SEQ ID NO: 739.

In another embodiment, the ISVD specifically binding to human PD-1 may have a sequence identity of more than 90%, such as more than 95% or even more than 99%, with SEQ ID NO: 740, wherein the CDRs are as defined above. In one embodiment, the ISVD specifically binding to PD-1 comprises or consists of the amino acid sequence of SEQ ID NO: 740.

In another embodiment, the ISVD specifically binding to human PD-1 may have a sequence identity of more than 90%, such as more than 95% or even more than 99%, with SEQ ID NO: 741, wherein the CDRs are as defined above. In one embodiment, the ISVD specifically binding to PD-1 comprises or consists of the amino acid sequence of SEQ ID NO: 741.

In another embodiment, the ISVD specifically binding to human PD-1 may have a sequence identity of more than 90%, such as more than 95% or even more than 99%, with SEQ ID NO: 742, wherein the CDRs are as defined above. In one embodiment, the ISVD specifically binding to PD-1 comprises or consists of the amino acid sequence of SEQ ID NO: 742.

In another embodiment, the ISVD specifically binding to human PD-1 may have a sequence identity of more than 90%, such as more than 95% or even more than 99%, with SEQ ID NO: 743, wherein the CDRs are as defined above. In one embodiment, the ISVD specifically binding to PD-1 comprises or consists of the amino acid sequence of SEQ ID NO: 743.

In another embodiment, the ISVD specifically binding to human PD-1 may have a sequence identity of more than 90%, such as more than 95% or even more than 99%, with SEQ ID NO: 744, wherein the CDRs are as defined above. In one embodiment, the ISVD specifically binding to PD-1 comprises or consists of the amino acid sequence of SEQ ID NO: 744.

In another embodiment, the ISVD specifically binding to human PD-1 may have a sequence identity of more than 90%, such as more than 95% or even more than 99%, with SEQ ID NO: 745, wherein the CDRs are as defined above. In one embodiment, the ISVD specifically binding to PD-1 comprises or consists of the amino acid sequence of SEQ ID NO: 745.

In another embodiment, the ISVD specifically binding to human PD-1 may have a sequence identity of more than 90%, such as more than 95% or even more than 99%, with SEQ ID NO: 746, wherein the CDRs are as defined above. In one embodiment, the ISVD specifically binding to PD-1 comprises or consists of the amino acid sequence of SEQ ID NO: 746.

In another embodiment, the ISVD specifically binding to human PD-1 may have a sequence identity of more than 90%, such as more than 95% or even more than 99%, with SEQ ID NO: 747, wherein the CDRs are as defined above. In one embodiment, the ISVD specifically binding to PD-1 comprises or consists of the amino acid sequence of SEQ ID NO: 747.

In another embodiment, the ISVD specifically binding to human PD-1 may have a sequence identity of more than 90%, such as more than 95% or even more than 99%, with SEQ ID NO: 748, wherein the CDRs are as defined above. In one embodiment, the ISVD specifically binding to PD-1 comprises or consists of the amino acid sequence of SEQ ID NO: 748.

In another embodiment, the ISVD specifically binding to human PD-1 may have a sequence identity of more than 90%, such as more than 95% or even more than 99%, with SEQ ID NO: 749, wherein the CDRs are as defined above. In one embodiment, the ISVD specifically binding to PD-1 comprises or consists of the amino acid sequence of SEQ ID NO: 749.

In another embodiment, the ISVD specifically binding to human PD-1 may have a sequence identity of more than 90%, such as more than 95% or even more than 99%, with SEQ ID NO: 750, wherein the CDRs are as defined above. In one embodiment, the ISVD specifically binding to PD-1 comprises or consists of the amino acid sequence of SEQ ID NO: 750.

In another embodiment, the ISVD specifically binding to human PD-1 may have a sequence identity of more than 90%, such as more than 95% or even more than 99%, with SEQ ID NO: 751, wherein the CDRs are as defined above. In one embodiment, the ISVD specifically binding to PD-1 comprises or consists of the amino acid sequence of SEQ ID NO: 751.

In another embodiment, the ISVD specifically binding to human PD-1 may have a sequence identity of more than 90%, such as more than 95% or even more than 99%, with SEQ ID NO: 752, wherein the CDRs are as defined above. In one embodiment, the ISVD specifically binding to PD-1 comprises or consists of the amino acid sequence of SEQ ID NO: 752.

In another embodiment, the ISVD specifically binding to human PD-1 may have a sequence identity of more than 90%, such as more than 95% or even more than 99%, with SEQ ID NO: 753, wherein the CDRs are as defined above. In one embodiment, the ISVD specifically binding to PD-1 comprises or consists of the amino acid sequence of SEQ ID NO: 753.

In another embodiment, the ISVD specifically binding to human PD-1 may have a sequence identity of more than 90%, such as more than 95% or even more than 99%, with SEQ ID NO: 754, wherein the CDRs are as defined above. In one embodiment, the ISVD specifically binding to PD-1 comprises or consists of the amino acid sequence of SEQ ID NO: 754.

In another embodiment, the ISVD specifically binding to human PD-1 may have a sequence identity of more than 90%, such as more than 95% or even more than 99%, with SEQ ID NO: 755, wherein the CDRs are as defined above. In one embodiment, the ISVD specifically binding to PD-1 comprises or consists of the amino acid sequence of SEQ ID NO: 755.

In another embodiment, the ISVD specifically binding to human PD-1 may have a sequence identity of more than 90%, such as more than 95% or even more than 99%, with SEQ ID NO: 756, wherein the CDRs are as defined above. In one embodiment, the ISVD specifically binding to PD-1 comprises or consists of the amino acid sequence of SEQ ID NO: 756.

In another embodiment, the ISVD specifically binding to human PD-1 may have a sequence identity of more than 90%, such as more than 95% or even more than 99%, with SEQ ID NO: 757, wherein the CDRs are as defined above. In one embodiment, the ISVD specifically binding to PD-1 comprises or consists of the amino acid sequence of SEQ ID NO: 757.

In another embodiment, the ISVD specifically binding to human PD-1 may have a sequence identity of more than 90%, such as more than 95% or even more than 99%, with SEQ ID NO: 758, wherein the CDRs are as defined above. In one embodiment, the ISVD specifically binding to PD-1 comprises or consists of the amino acid sequence of SEQ ID NO: 758.

In another embodiment, the ISVD specifically binding to human PD-1 may have a sequence identity of more than 90%, such as more than 95% or even more than 99%, with SEQ ID NO: 759, wherein the CDRs are as defined above. In one embodiment, the ISVD specifically binding to PD-1 comprises or consists of the amino acid sequence of SEQ ID NO: 759.

In another embodiment, the ISVD specifically binding to human PD-1 may have a sequence identity of more than 90%, such as more than 95% or even more than 99%, with SEQ ID NO: 760, wherein the CDRs are as defined above. In one embodiment, the ISVD specifically binding to PD-1 comprises or consists of the amino acid sequence of SEQ ID NO: 760.

In another embodiment, the ISVD specifically binding to human PD-1 may have a sequence identity of more than 90%, such as more than 95% or even more than 99%, with SEQ ID NO: 761, wherein the CDRs are as defined above. In one embodiment, the ISVD specifically binding to PD-1 comprises or consists of the amino acid sequence of SEQ ID NO: 761.

In another embodiment, the ISVD specifically binding to human PD-1 may have a sequence identity of more than 90%, such as more than 95% or even more than 99%, with SEQ ID NO: 762, wherein the CDRs are as defined above. In one embodiment, the ISVD specifically binding to PD-1 comprises or consists of the amino acid sequence of SEQ ID NO: 762.

In another embodiment, the ISVD specifically binding to human PD-1 may have a sequence identity of more than 90%, such as more than 95% or even more than 99%, with SEQ ID NO: 763, wherein the CDRs are as defined above. In one embodiment, the ISVD specifically binding to PD-1 comprises or consists of the amino acid sequence of SEQ ID NO: 763.

In another embodiment, the ISVD specifically binding to human PD-1 may have a sequence identity of more than 90%, such as more than 95% or even more than 99%, with SEQ ID NO: 764, wherein the CDRs are as defined above. In one embodiment, the ISVD specifically binding to PD-1 comprises or consists of the amino acid sequence of SEQ ID NO: 764.

In another embodiment, the ISVD specifically binding to human PD-1 may have a sequence identity of more than 90%, such as more than 95% or even more than 99%, with SEQ ID NO: 765, wherein the CDRs are as defined above. In one embodiment, the ISVD specifically binding to PD-1 comprises or consists of the amino acid sequence of SEQ ID NO: 765.

In another embodiment, the ISVD specifically binding to human PD-1 may have a sequence identity of more than 90%, such as more than 95% or even more than 99%, with SEQ ID NO: 766, wherein the CDRs are as defined above. In one embodiment, the ISVD specifically binding to PD-1 comprises or consists of the amino acid sequence of SEQ ID NO: 766.

In another embodiment, the ISVD specifically binding to human PD-1 may have a sequence identity of more than 90%, such as more than 95% or even more than 99%, with SEQ ID NO: 767, wherein the CDRs are as defined above. In one embodiment, the ISVD specifically binding to PD-1 comprises or consists of the amino acid sequence of SEQ ID NO: 767.

In another embodiment, the ISVD specifically binding to human PD-1 may have a sequence identity of more than 90%, such as more than 95% or even more than 99%, with SEQ ID NO: 768, wherein the CDRs are as defined above. In one embodiment, the ISVD specifically binding to PD-1 comprises or consists of the amino acid sequence of SEQ ID NO: 768.

In another embodiment, the ISVD specifically binding to human PD-1 may have a sequence identity of more than 90%, such as more than 95% or even more than 99%, with SEQ ID NO: 769, wherein the CDRs are as defined above. In one embodiment, the ISVD specifically binding to PD-1 comprises or consists of the amino acid sequence of SEQ ID NO: 769.

In another embodiment, the ISVD specifically binding to human PD-1 may have a sequence identity of more than 90%, such as more than 95% or even more than 99%, with SEQ ID NO: 770, wherein the CDRs are as defined above. In one embodiment, the ISVD specifically binding to PD-1 comprises or consists of the amino acid sequence of SEQ ID NO: 770.

In another embodiment, the ISVD specifically binding to human PD-1 may have a sequence identity of more than 90%, such as more than 95% or even more than 99%, with SEQ ID NO: 771, wherein the CDRs are as defined above. In one embodiment, the ISVD specifically binding to PD-1 comprises or consists of the amino acid sequence of SEQ ID NO: 771.

In another embodiment, the ISVD specifically binding to human PD-1 may have a sequence identity of more than 90%, such as more than 95% or even more than 99%, with SEQ ID NO: 772, wherein the CDRs are as defined above. In one embodiment, the ISVD specifically binding to PD-1 comprises or consists of the amino acid sequence of SEQ ID NO: 772.

In another embodiment, the ISVD specifically binding to human PD-1 may have a sequence identity of more than 90%, such as more than 95% or even more than 99%, with SEQ ID NO: 773, wherein the CDRs are as defined above. In one embodiment, the ISVD specifically binding to PD-1 comprises or consists of the amino acid sequence of SEQ ID NO: 773.

In another embodiment, the ISVD specifically binding to human PD-1 may have a sequence identity of more than 90%, such as more than 95% or even more than 99%, with SEQ ID NO: 774, wherein the CDRs are as defined above. In one embodiment, the ISVD specifically binding to PD-1 comprises or consists of the amino acid sequence of SEQ ID NO: 774.

In another embodiment, the ISVD specifically binding to human PD-1 may have a sequence identity of more than 90%, such as more than 95% or even more than 99%, with SEQ ID NO: 775, wherein the CDRs are as defined above. In one embodiment, the ISVD specifically binding to PD-1 comprises or consists of the amino acid sequence of SEQ ID NO: 775.

In another embodiment, the ISVD specifically binding to human PD-1 may have a sequence identity of more than 90%, such as more than 95% or even more than 99%, with SEQ ID NO: 776, wherein the CDRs are as defined above. In one embodiment, the ISVD specifically binding to PD-1 comprises or consists of the amino acid sequence of SEQ ID NO: 776.

In another embodiment, the ISVD specifically binding to human PD-1 may have a sequence identity of more than 90%, such as more than 95% or even more than 99%, with SEQ ID NO: 777, wherein the CDRs are as defined above. In one embodiment, the ISVD specifically binding to PD-1 comprises or consists of the amino acid sequence of SEQ ID NO: 777.

In another embodiment, the ISVD specifically binding to human PD-1 may have a sequence identity of more than 90%, such as more than 95% or even more than 99%, with SEQ ID NO: 778, wherein the CDRs are as defined above. In one embodiment, the ISVD specifically binding to PD-1 comprises or consists of the amino acid sequence of SEQ ID NO: 778.

In another embodiment, the ISVD specifically binding to human PD-1 may have a sequence identity of more than 90%, such as more than 95% or even more than 99%, with SEQ ID NO: 779, wherein the CDRs are as defined above. In one embodiment, the ISVD specifically binding to PD-1 comprises or consists of the amino acid sequence of SEQ ID NO: 779.

In another embodiment, the ISVD specifically binding to human PD-1 may have a sequence identity of more than 90%, such as more than 95% or even more than 99%, with SEQ ID NO: 780, wherein the CDRs are as defined above. In one embodiment, the ISVD specifically binding to PD-1 comprises or consists of the amino acid sequence of SEQ ID NO: 780.

In another embodiment, the ISVD specifically binding to human PD-1 may have a sequence identity of more than 90%, such as more than 95% or even more than 99%, with SEQ ID NO: 781, wherein the CDRs are as defined above. In one embodiment, the ISVD specifically binding to PD-1 comprises or consists of the amino acid sequence of SEQ ID NO: 781.

In another embodiment, the ISVD specifically binding to human PD-1 may have a sequence identity of more than 90%, such as more than 95% or even more than 99%, with SEQ ID NO: 782, wherein the CDRs are as defined above. In one embodiment, the ISVD specifically binding to PD-1 comprises or consists of the amino acid sequence of SEQ ID NO: 782.

In another embodiment, the ISVD specifically binding to human PD-1 may have a sequence identity of more than 90%, such as more than 95% or even more than 99%, with SEQ ID NO: 783, wherein the CDRs are as defined above. In one embodiment, the ISVD specifically binding to PD-1 comprises or consists of the amino acid sequence of SEQ ID NO: 783.

In another embodiment, the ISVD specifically binding to human PD-1 may have a sequence identity of more than 90%, such as more than 95% or even more than 99%, with SEQ ID NO: 784, wherein the CDRs are as defined above. In one embodiment, the ISVD specifically binding to PD-1 comprises or consists of the amino acid sequence of SEQ ID NO: 784.

In another embodiment, the ISVD specifically binding to human PD-1 may have a sequence identity of more than 90%, such as more than 95% or even more than 99%, with SEQ ID NO: 785, wherein the CDRs are as defined above. In one embodiment, the ISVD specifically binding to PD-1 comprises or consists of the amino acid sequence of SEQ ID NO: 785.

In another embodiment, the ISVD specifically binding to human PD-1 may have a sequence identity of more than 90%, such as more than 95% or even more than 99%, with SEQ ID NO: 786, wherein the CDRs are as defined above. In one embodiment, the ISVD specifically binding to PD-1 comprises or consists of the amino acid sequence of SEQ ID NO: 786.

In another embodiment, the ISVD specifically binding to human PD-1 may have a sequence identity of more than 90%, such as more than 95% or even more than 99%, with SEQ ID NO: 787, wherein the CDRs are as defined above. In one embodiment, the ISVD specifically binding to PD-1 comprises or consists of the amino acid sequence of SEQ ID NO: 787.

In another embodiment, the ISVD specifically binding to human PD-1 may have a sequence identity of more than 90%, such as more than 95% or even more than 99%, with SEQ ID NO: 788, wherein the CDRs are as defined above. In one embodiment, the ISVD specifically binding to PD-1 comprises or consists of the amino acid sequence of SEQ ID NO: 788.

In another embodiment, the ISVD specifically binding to human PD-1 may have a sequence identity of more than 90%, such as more than 95% or even more than 99%, with SEQ ID NO: 789, wherein the CDRs are as defined above. In one embodiment, the ISVD specifically binding to PD-1 comprises or consists of the amino acid sequence of SEQ ID NO: 789.

In another embodiment, the ISVD specifically binding to human PD-1 may have a sequence identity of more than 90%, such as more than 95% or even more than 99%, with SEQ ID NO: 790, wherein the CDRs are as defined above. In one embodiment, the ISVD specifically binding to PD-1 comprises or consists of the amino acid sequence of SEQ ID NO: 790.

In another embodiment, the ISVD specifically binding to human PD-1 may have a sequence identity of more than 90%, such as more than 95% or even more than 99%, with SEQ ID NO: 791, wherein the CDRs are as defined above. In one embodiment, the ISVD specifically binding to PD-1 comprises or consists of the amino acid sequence of SEQ ID NO: 791.

In another embodiment, the ISVD specifically binding to human PD-1 may have a sequence identity of more than 90%, such as more than 95% or even more than 99%, with SEQ ID NO: 792, wherein the CDRs are as defined above. In one embodiment, the ISVD specifically binding to PD-1 comprises or consists of the amino acid sequence of SEQ ID NO: 792.

In another embodiment, the ISVD specifically binding to human PD-1 may have a sequence identity of more than 90%, such as more than 95% or even more than 99%, with SEQ ID NO: 793, wherein the CDRs are as defined above. In one embodiment, the ISVD specifically binding to PD-1 comprises or consists of the amino acid sequence of SEQ ID NO: 793.

In another embodiment, the ISVD specifically binding to human PD-1 may have a sequence identity of more than 90%, such as more than 95% or even more than 99%, with SEQ ID NO: 794, wherein the CDRs are as defined above. In one embodiment, the ISVD specifically binding to PD-1 comprises or consists of the amino acid sequence of SEQ ID NO: 794.

In another embodiment, the ISVD specifically binding to human PD-1 may have a sequence identity of more than 90%, such as more than 95% or even more than 99%, with SEQ ID NO: 795, wherein the CDRs are as defined above. In one embodiment, the ISVD specifically binding to PD-1 comprises or consists of the amino acid sequence of SEQ ID NO: 795.

In another embodiment, the ISVD specifically binding to human PD-1 may have a sequence identity of more than 90%, such as more than 95% or even more than 99%, with SEQ ID NO: 796, wherein the CDRs are as defined above. In one embodiment, the ISVD specifically binding to PD-1 comprises or consists of the amino acid sequence of SEQ ID NO: 796.

In another embodiment, the ISVD specifically binding to human PD-1 may have a sequence identity of more than 90%, such as more than 95% or even more than 99%, with SEQ ID NO: 797, wherein the CDRs are as defined above. In one embodiment, the ISVD specifically binding to PD-1 comprises or consists of the amino acid sequence of SEQ ID NO: 797.

In another embodiment, the ISVD specifically binding to human PD-1 may have a sequence identity of more than 90%, such as more than 95% or even more than 99%, with SEQ ID NO: 798, wherein the CDRs are as defined above. In one embodiment, the ISVD specifically binding to PD-1 comprises or consists of the amino acid sequence of SEQ ID NO: 798.

In another embodiment, the ISVD specifically binding to human PD-1 may have a sequence identity of more than 90%, such as more than 95% or even more than 99%, with SEQ ID NO: 799, wherein the CDRs are as defined above. In one embodiment, the ISVD specifically binding to PD-1 comprises or consists of the amino acid sequence of SEQ ID NO: 799.

In another embodiment, the ISVD specifically binding to human PD-1 may have a sequence identity of more than 90%, such as more than 95% or even more than 99%, with SEQ ID NO: 800, wherein the CDRs are as defined above. In one embodiment, the ISVD specifically binding to PD-1 comprises or consists of the amino acid sequence of SEQ ID NO: 800.

In another embodiment, the ISVD specifically binding to human PD-1 may have a sequence identity of more than 90%, such as more than 95% or even more than 99%, with SEQ ID NO: 801, wherein the CDRs are as defined above. In one embodiment, the ISVD specifically binding to PD-1 comprises or consists of the amino acid sequence of SEQ ID NO: 801.

In another embodiment, the ISVD specifically binding to human PD-1 may have a sequence identity of more than 90%, such as more than 95% or even more than 99%, with SEQ ID NO: 802, wherein the CDRs are as defined above. In one embodiment, the ISVD specifically binding to PD-1 comprises or consists of the amino acid sequence of SEQ ID NO: 802.

In another embodiment, the ISVD specifically binding to human PD-1 may have a sequence identity of more than 90%, such as more than 95% or even more than 99%, with SEQ ID NO: 803, wherein the CDRs are as defined above. In one embodiment, the ISVD specifically binding to PD-1 comprises or consists of the amino acid sequence of SEQ ID NO: 803.

In another embodiment, the ISVD specifically binding to human PD-1 may have a sequence identity of more than 90%, such as more than 95% or even more than 99%, with SEQ ID NO: 804, wherein the CDRs are as defined above. In one embodiment, the ISVD specifically binding to PD-1 comprises or consists of the amino acid sequence of SEQ ID NO: 804.

In another embodiment, the ISVD specifically binding to human PD-1 may have a sequence identity of more than 90%, such as more than 95% or even more than 99%, with SEQ ID NO: 805, wherein the CDRs are as defined above. In one embodiment, the ISVD specifically binding to PD-1 comprises or consists of the amino acid sequence of SEQ ID NO: 805.

In another embodiment, the ISVD specifically binding to human PD-1 may have a sequence identity of more than 90%, such as more than 95% or even more than 99%, with SEQ ID NO: 806, wherein the CDRs are as defined above. In one embodiment, the ISVD specifically binding to PD-1 comprises or consists of the amino acid sequence of SEQ ID NO: 806.

In another embodiment, the ISVD specifically binding to human PD-1 may have a sequence identity of more than 90%, such as more than 95% or even more than 99%, with SEQ ID NO: 807, wherein the CDRs are as defined above. In one embodiment, the ISVD specifically binding to PD-1 comprises or consists of the amino acid sequence of SEQ ID NO: 807.

In another embodiment, the ISVD specifically binding to human PD-1 may have a sequence identity of more than 90%, such as more than 95% or even more than 99%, with SEQ ID NO: 808, wherein the CDRs are as defined above. In one embodiment, the ISVD specifically binding to PD-1 comprises or consists of the amino acid sequence of SEQ ID NO: 808.

In another embodiment, the ISVD specifically binding to human PD-1 may have a sequence identity of more than 90%, such as more than 95% or even more than 99%, with SEQ ID NO: 809, wherein the CDRs are as defined above. In one embodiment, the ISVD specifically binding to PD-1 comprises or consists of the amino acid sequence of SEQ ID NO: 809.

In another embodiment, the ISVD specifically binding to human PD-1 may have a sequence identity of more than 90%, such as more than 95% or even more than 99%, with SEQ ID NO: 810, wherein the CDRs are as defined above. In one embodiment, the ISVD specifically binding to PD-1 comprises or consists of the amino acid sequence of SEQ ID NO: 810.

In another embodiment, the ISVD specifically binding to human PD-1 may have a sequence identity of more than 90%, such as more than 95% or even more than 99%, with SEQ ID NO: 811, wherein the CDRs are as defined above. In one embodiment, the ISVD specifically binding to PD-1 comprises or consists of the amino acid sequence of SEQ ID NO: 811.

In another embodiment, the ISVD specifically binding to human PD-1 may have a sequence identity of more than 90%, such as more than 95% or even more than 99%, with SEQ ID NO: 812, wherein the CDRs are as defined above. In one embodiment, the ISVD specifically binding to PD-1 comprises or consists of the amino acid sequence of SEQ ID NO: 812.

In another embodiment, the ISVD specifically binding to human PD-1 may have a sequence identity of more than 90%, such as more than 95% or even more than 99%, with SEQ ID NO: 813, wherein the CDRs are as defined above. In one embodiment, the ISVD specifically binding to PD-1 comprises or consists of the amino acid sequence of SEQ ID NO: 813.

In another embodiment, the ISVD specifically binding to human PD-1 may have a sequence identity of more than 90%, such as more than 95% or even more than 99%, with SEQ ID NO: 814, wherein the CDRs are as defined above. In one embodiment, the ISVD specifically binding to PD-1 comprises or consists of the amino acid sequence of SEQ ID NO: 814.

In another embodiment, the ISVD specifically binding to human PD-1 may have a sequence identity of more than 90%, such as more than 95% or even more than 99%, with SEQ ID NO: 815, wherein the CDRs are as defined above. In one embodiment, the ISVD specifically binding to PD-1 comprises or consists of the amino acid sequence of SEQ ID NO: 815.

In another embodiment, the ISVD specifically binding to human PD-1 may have a sequence identity of more than 90%, such as more than 95% or even more than 99%, with SEQ ID NO: 816, wherein the CDRs are as defined above. In one embodiment, the ISVD specifically binding to PD-1 comprises or consists of the amino acid sequence of SEQ ID NO: 816.

In another embodiment, the ISVD specifically binding to human PD-1 may have a sequence identity of more than 90%, such as more than 95% or even more than 99%, with SEQ ID NO: 817, wherein the CDRs are as defined above. In one embodiment, the ISVD specifically binding to PD-1 comprises or consists of the amino acid sequence of SEQ ID NO: 817.

In another embodiment, the ISVD specifically binding to human PD-1 may have a sequence identity of more than 90%, such as more than 95% or even more than 99%, with SEQ ID NO: 818, wherein the CDRs are as defined above. In one embodiment, the ISVD specifically binding to PD-1 comprises or consists of the amino acid sequence of SEQ ID NO: 818.

In another embodiment, the ISVD specifically binding to human PD-1 may have a sequence identity of more than 90%, such as more than 95% or even more than 99%, with SEQ ID NO: 819, wherein the CDRs are as defined above. In one embodiment, the ISVD specifically binding to PD-1 comprises or consists of the amino acid sequence of SEQ ID NO: 819.

In another embodiment, the ISVD specifically binding to human PD-1 may have a sequence identity of more than 90%, such as more than 95% or even more than 99%, with SEQ ID NO: 820, wherein the CDRs are as defined above. In one embodiment, the ISVD specifically binding to PD-1 comprises or consists of the amino acid sequence of SEQ ID NO: 820.

In another embodiment, the ISVD specifically binding to human PD-1 may have a sequence identity of more than 90%, such as more than 95% or even more than 99%, with SEQ ID NO: 821, wherein the CDRs are as defined above. In one embodiment, the ISVD specifically binding to PD-1 comprises or consists of the amino acid sequence of SEQ ID NO: 821.

In another embodiment, the ISVD specifically binding to human PD-1 may have a sequence identity of more than 90%, such as more than 95% or even more than 99%, with SEQ ID NO: 822, wherein the CDRs are as defined above. In one embodiment, the ISVD specifically binding to PD-1 comprises or consists of the amino acid sequence of SEQ ID NO: 822.

In another embodiment, the ISVD specifically binding to human PD-1 may have a sequence identity of more than 90%, such as more than 95% or even more than 99%, with SEQ ID NO: 823, wherein the CDRs are as defined above. In one embodiment, the ISVD specifically binding to PD-1 comprises or consists of the amino acid sequence of SEQ ID NO: 823.

In another embodiment, the ISVD specifically binding to human PD-1 may have a sequence identity of more than 90%, such as more than 95% or even more than 99%, with SEQ ID NO: 824, wherein the CDRs are as defined above. In one embodiment, the ISVD specifically binding to PD-1 comprises or consists of the amino acid sequence of SEQ ID NO: 824.

In another embodiment, the ISVD specifically binding to human PD-1 may have a sequence identity of more than 90%, such as more than 95% or even more than 99%, with SEQ ID NO: 825, wherein the CDRs are as defined above. In one embodiment, the ISVD specifically binding to PD-1 comprises or consists of the amino acid sequence of SEQ ID NO: 825.

In another embodiment, the ISVD specifically binding to human PD-1 may have a sequence identity of more than 90%, such as more than 95% or even more than 99%, with SEQ ID NO: 826, wherein the CDRs are as defined above. In one embodiment, the ISVD specifically binding to PD-1 comprises or consists of the amino acid sequence of SEQ ID NO: 826.

In another embodiment, the ISVD specifically binding to human PD-1 may have a sequence identity of more than 90%, such as more than 95% or even more than 99%, with SEQ ID NO: 827, wherein the CDRs are as defined above. In one embodiment, the ISVD specifically binding to PD-1 comprises or consists of the amino acid sequence of SEQ ID NO: 827.

In another embodiment, the ISVD specifically binding to human PD-1 may have a sequence identity of more than 90%, such as more than 95% or even more than 99%, with SEQ ID NO: 828, wherein the CDRs are as defined above. In one embodiment, the ISVD specifically binding to PD-1 comprises or consists of the amino acid sequence of SEQ ID NO: 828.

In another embodiment, the ISVD specifically binding to human PD-1 may have a sequence identity of more than 90%, such as more than 95% or even more than 99%, with SEQ ID NO: 829, wherein the CDRs are as defined above. In one embodiment, the ISVD specifically binding to PD-1 comprises or consists of the amino acid sequence of SEQ ID NO: 829.

In another embodiment, the ISVD specifically binding to human PD-1 may have a sequence identity of more than 90%, such as more than 95% or even more than 99%, with SEQ ID NO: 830, wherein the CDRs are as defined above. In one embodiment, the ISVD specifically binding to PD-1 comprises or consists of the amino acid sequence of SEQ ID NO: 830.

In another embodiment, the ISVD specifically binding to human PD-1 may have a sequence identity of more than 90%, such as more than 95% or even more than 99%, with SEQ ID NO: 831, wherein the CDRs are as defined above. In one embodiment, the ISVD specifically binding to PD-1 comprises or consists of the amino acid sequence of SEQ ID NO: 831.

In another embodiment, the ISVD specifically binding to human PD-1 may have a sequence identity of more than 90%, such as more than 95% or even more than 99%, with SEQ ID NO: 832, wherein the CDRs are as defined above. In one embodiment, the ISVD specifically binding to PD-1 comprises or consists of the amino acid sequence of SEQ ID NO: 832.

In another embodiment, the ISVD specifically binding to human PD-1 may have a sequence identity of more than 90%, such as more than 95% or even more than 99%, with SEQ ID NO: 833, wherein the CDRs are as defined above. In one embodiment, the ISVD specifically binding to PD-1 comprises or consists of the amino acid sequence of SEQ ID NO: 833.

In another embodiment, the ISVD specifically binding to human PD-1 may have a sequence identity of more than 90%, such as more than 95% or even more than 99%, with SEQ ID NO: 834, wherein the CDRs are as defined above. In one embodiment, the ISVD specifically binding to PD-1 comprises or consists of the amino acid sequence of SEQ ID NO: 834.

In another embodiment, the ISVD specifically binding to human PD-1 may have a sequence identity of more than 90%, such as more than 95% or even more than 99%, with SEQ ID NO: 835, wherein the CDRs are as defined above. In one embodiment, the ISVD specifically binding to PD-1 comprises or consists of the amino acid sequence of SEQ ID NO: 835.

In another embodiment, the ISVD specifically binding to human PD-1 may have a sequence identity of more than 90%, such as more than 95% or even more than 99%, with SEQ ID NO: 836, wherein the CDRs are as defined above. In one embodiment, the ISVD specifically binding to PD-1 comprises or consists of the amino acid sequence of SEQ ID NO: 836.

In another embodiment, the ISVD specifically binding to human PD-1 may have a sequence identity of more than 90%, such as more than 95% or even more than 99%, with SEQ ID NO: 837, wherein the CDRs are as defined above. In one embodiment, the ISVD specifically binding to PD-1 comprises or consists of the amino acid sequence of SEQ ID NO: 837.

In another embodiment, the ISVD specifically binding to human PD-1 may have a sequence identity of more than 90%, such as more than 95% or even more than 99%, with SEQ ID NO: 838, wherein the CDRs are as defined above. In one embodiment, the ISVD specifically binding to PD-1 comprises or consists of the amino acid sequence of SEQ ID NO: 838.

In another embodiment, the ISVD specifically binding to human PD-1 may have a sequence identity of more than 90%, such as more than 95% or even more than 99%, with SEQ ID NO: 839, wherein the CDRs are as defined above. In one embodiment, the ISVD specifically binding to PD-1 comprises or consists of the amino acid sequence of SEQ ID NO: 839.

In another embodiment, the ISVD specifically binding to human PD-1 may have a sequence identity of more than 90%, such as more than 95% or even more than 99%, with SEQ ID NO: 840, wherein the CDRs are as defined above. In one embodiment, the ISVD specifically binding to PD-1 comprises or consists of the amino acid sequence of SEQ ID NO: 840.

In another embodiment, the ISVD specifically binding to human PD-1 may have a sequence identity of more than 90%, such as more than 95% or even more than 99%, with SEQ ID NO: 841, wherein the CDRs are as defined above. In one embodiment, the ISVD specifically binding to PD-1 comprises or consists of the amino acid sequence of SEQ ID NO: 841.

In another embodiment, the ISVD specifically binding to human PD-1 may have a sequence identity of more than 90%, such as more than 95% or even more than 99%, with SEQ ID NO: 842, wherein the CDRs are as defined above. In one embodiment, the ISVD specifically binding to PD-1 comprises or consists of the amino acid sequence of SEQ ID NO: 842.

In another embodiment, the ISVD specifically binding to human PD-1 may have a sequence identity of more than 90%, such as more than 95% or even more than 99%, with SEQ ID NO: 843, wherein the CDRs are as defined above. In one embodiment, the ISVD specifically binding to PD-1 comprises or consists of the amino acid sequence of SEQ ID NO: 843.

In another embodiment, the ISVD specifically binding to human PD-1 may have a sequence identity of more than 90%, such as more than 95% or even more than 99%, with SEQ ID NO: 844, wherein the CDRs are as defined above. In one embodiment, the ISVD specifically binding to PD-1 comprises or consists of the amino acid sequence of SEQ ID NO: 844.

In another embodiment, the ISVD specifically binding to human PD-1 may have a sequence identity of more than 90%, such as more than 95% or even more than 99%, with SEQ ID NO: 845, wherein the CDRs are as defined above. In one embodiment, the ISVD specifically binding to PD-1 comprises or consists of the amino acid sequence of SEQ ID NO: 845.

In another embodiment, the ISVD specifically binding to human PD-1 may have a sequence identity of more than 90%, such as more than 95% or even more than 99%, with SEQ ID NO: 846, wherein the CDRs are as defined above. In one embodiment, the ISVD specifically binding to PD-1 comprises or consists of the amino acid sequence of SEQ ID NO: 846.

In another embodiment, the ISVD specifically binding to human PD-1 may have a sequence identity of more than 90%, such as more than 95% or even more than 99%, with SEQ ID NO: 847, wherein the CDRs are as defined above. In one embodiment, the ISVD specifically binding to PD-1 comprises or consists of the amino acid sequence of SEQ ID NO: 847.

In another embodiment, the ISVD specifically binding to human PD-1 may have a sequence identity of more than 90%, such as more than 95% or even more than 99%, with SEQ ID NO: 848, wherein the CDRs are as defined above. In one embodiment, the ISVD specifically binding to PD-1 comprises or consists of the amino acid sequence of SEQ ID NO: 848.

In another embodiment, the ISVD specifically binding to human PD-1 may have a sequence identity of more than 90%, such as more than 95% or even more than 99%, with SEQ ID NO: 849, wherein the CDRs are as defined above. In one embodiment, the ISVD specifically binding to PD-1 comprises or consists of the amino acid sequence of SEQ ID NO: 849.

In another embodiment, the ISVD specifically binding to human PD-1 may have a sequence identity of more than 90%, such as more than 95% or even more than 99%, with SEQ ID NO: 850, wherein the CDRs are as defined above. In one embodiment, the ISVD specifically binding to PD-1 comprises or consists of the amino acid sequence of SEQ ID NO: 850.

In another embodiment, the ISVD specifically binding to human PD-1 may have a sequence identity of more than 90%, such as more than 95% or even more than 99%, with SEQ ID NO: 851, wherein the CDRs are as defined above. In one embodiment, the ISVD specifically binding to PD-1 comprises or consists of the amino acid sequence of SEQ ID NO: 851.

In another embodiment, the ISVD specifically binding to human PD-1 may have a sequence identity of more than 90%, such as more than 95% or even more than 99%, with SEQ ID NO: 852, wherein the CDRs are as defined above. In one embodiment, the ISVD specifically binding to PD-1 comprises or consists of the amino acid sequence of SEQ ID NO: 852.

In another embodiment, the ISVD specifically binding to human PD-1 may have a sequence identity of more than 90%, such as more than 95% or even more than 99%, with SEQ ID NO: 853, wherein the CDRs are as defined above. In one embodiment, the ISVD specifically binding to PD-1 comprises or consists of the amino acid sequence of SEQ ID NO: 853.

In another embodiment, the ISVD specifically binding to human PD-1 may have a sequence identity of more than 90%, such as more than 95% or even more than 99%, with SEQ ID NO: 854, wherein the CDRs are as defined above. In one embodiment, the ISVD specifically binding to PD-1 comprises or consists of the amino acid sequence of SEQ ID NO: 854.

In another embodiment, the ISVD specifically binding to human PD-1 may have a sequence identity of more than 90%, such as more than 95% or even more than 99%, with SEQ ID NO: 855, wherein the CDRs are as defined above. In one embodiment, the ISVD specifically binding to PD-1 comprises or consists of the amino acid sequence of SEQ ID NO: 855.

In another embodiment, the ISVD specifically binding to human PD-1 may have a sequence identity of more than 90%, such as more than 95% or even more than 99%, with SEQ ID NO: 856, wherein the CDRs are as defined above. In one embodiment, the ISVD specifically binding to PD-1 comprises or consists of the amino acid sequence of SEQ ID NO: 856.

In another embodiment, the ISVD specifically binding to human PD-1 may have a sequence identity of more than 90%, such as more than 95% or even more than 99%, with SEQ ID NO: 857, wherein the CDRs are as defined above. In one embodiment, the ISVD specifically binding to PD-1 comprises or consists of the amino acid sequence of SEQ ID NO: 857.

In another embodiment, the ISVD specifically binding to human PD-1 may have a sequence identity of more than 90%, such as more than 95% or even more than 99%, with SEQ ID NO: 858, wherein the CDRs are as defined above. In one embodiment, the ISVD specifically binding to PD-1 comprises or consists of the amino acid sequence of SEQ ID NO: 858.

In another embodiment, the ISVD specifically binding to human PD-1 may have a sequence identity of more than 90%, such as more than 95% or even more than 99%, with SEQ ID NO: 859, wherein the CDRs are as defined above. In one embodiment, the ISVD specifically binding to PD-1 comprises or consists of the amino acid sequence of SEQ ID NO: 859.

In another embodiment, the ISVD specifically binding to human PD-1 may have a sequence identity of more than 90%, such as more than 95% or even more than 99%, with SEQ ID NO: 860, wherein the CDRs are as defined above. In one embodiment, the ISVD specifically binding to PD-1 comprises or consists of the amino acid sequence of SEQ ID NO: 860.

In another embodiment, the ISVD specifically binding to human PD-1 may have a sequence identity of more than 90%, such as more than 95% or even more than 99%, with SEQ ID NO: 861, wherein the CDRs are as defined above. In one embodiment, the ISVD specifically binding to PD-1 comprises or consists of the amino acid sequence of SEQ ID NO: 861.

In another embodiment, the ISVD specifically binding to human PD-1 may have a sequence identity of more than 90%, such as more than 95% or even more than 99%, with SEQ ID NO: 862, wherein the CDRs are as defined above. In one embodiment, the ISVD specifically binding to PD-1 comprises or consists of the amino acid sequence of SEQ ID NO: 862.

In another embodiment, the ISVD specifically binding to human PD-1 may have a sequence identity of more than 90%, such as more than 95% or even more than 99%, with SEQ ID NO: 863, wherein the CDRs are as defined above. In one embodiment, the ISVD specifically binding to PD-1 comprises or consists of the amino acid sequence of SEQ ID NO: 863.

In another embodiment, the ISVD specifically binding to human PD-1 may have a sequence identity of more than 90%, such as more than 95% or even more than 99%, with SEQ ID NO: 864, wherein the CDRs are as defined above. In one embodiment, the ISVD specifically binding to PD-1 comprises or consists of the amino acid sequence of SEQ ID NO: 864.

In another embodiment, the ISVD specifically binding to human PD-1 may have a sequence identity of more than 90%, such as more than 95% or even more than 99%, with SEQ ID NO: 865, wherein the CDRs are as defined above. In one embodiment, the ISVD specifically binding to PD-1 comprises or consists of the amino acid sequence of SEQ ID NO: 865.

In another embodiment, the ISVD specifically binding to human PD-1 may have a sequence identity of more than 90%, such as more than 95% or even more than 99%, with SEQ ID NO: 866, wherein the CDRs are as defined above. In one embodiment, the ISVD specifically binding to PD-1 comprises or consists of the amino acid sequence of SEQ ID NO: 866.

In another embodiment, the ISVD specifically binding to human PD-1 may have a sequence identity of more than 90%, such as more than 95% or even more than 99%, with SEQ ID NO: 867, wherein the CDRs are as defined above. In one embodiment, the ISVD specifically binding to PD-1 comprises or consists of the amino acid sequence of SEQ ID NO: 867.

In another embodiment, the ISVD specifically binding to human PD-1 may have a sequence identity of more than 90%, such as more than 95% or even more than 99%, with SEQ ID NO: 868, wherein the CDRs are as defined above. In one embodiment, the ISVD specifically binding to PD-1 comprises or consists of the amino acid sequence of SEQ ID NO: 868.

In another embodiment, the ISVD specifically binding to human PD-1 may have a sequence identity of more than 90%, such as more than 95% or even more than 99%, with SEQ ID NO: 869, wherein the CDRs are as defined above. In one embodiment, the ISVD specifically binding to PD-1 comprises or consists of the amino acid sequence of SEQ ID NO: 869.

In another embodiment, the ISVD specifically binding to human PD-1 may have a sequence identity of more than 90%, such as more than 95% or even more than 99%, with SEQ ID NO: 870, wherein the CDRs are as defined above. In one embodiment, the ISVD specifically binding to PD-1 comprises or consists of the amino acid sequence of SEQ ID NO: 870.

In another embodiment, the ISVD specifically binding to human PD-1 may have a sequence identity of more than 90%, such as more than 95% or even more than 99%, with SEQ ID NO: 871, wherein the CDRs are as defined above. In one embodiment, the ISVD specifically binding to PD-1 comprises or consists of the amino acid sequence of SEQ ID NO: 871.

In another embodiment, the ISVD specifically binding to human PD-1 may have a sequence identity of more than 90%, such as more than 95% or even more than 99%, with SEQ ID NO: 872, wherein the CDRs are as defined above. In one embodiment, the ISVD specifically binding to PD-1 comprises or consists of the amino acid sequence of SEQ ID NO: 872.

In another embodiment, the ISVD specifically binding to human PD-1 may have a sequence identity of more than 90%, such as more than 95% or even more than 99%, with SEQ ID NO: 873, wherein the CDRs are as defined above. In one embodiment, the ISVD specifically binding to PD-1 comprises or consists of the amino acid sequence of SEQ ID NO: 873.

In another embodiment, the ISVD specifically binding to human PD-1 may have a sequence identity of more than 90%, such as more than 95% or even more than 99%, with SEQ ID NO: 874, wherein the CDRs are as defined above. In one embodiment, the ISVD specifically binding to PD-1 comprises or consists of the amino acid sequence of SEQ ID NO: 874.

In another embodiment, the ISVD specifically binding to human PD-1 may have a sequence identity of more than 90%, such as more than 95% or even more than 99%, with SEQ ID NO: 875, wherein the CDRs are as defined above. In one embodiment, the ISVD specifically binding to PD-1 comprises or consists of the amino acid sequence of SEQ ID NO: 875.

In another embodiment, the ISVD specifically binding to human PD-1 may have a sequence identity of more than 90%, such as more than 95% or even more than 99%, with SEQ ID NO: 876, wherein the CDRs are as defined above. In one embodiment, the ISVD specifically binding to PD-1 comprises or consists of the amino acid sequence of SEQ ID NO: 876.

In another embodiment, the ISVD specifically binding to human PD-1 may have a sequence identity of more than 90%, such as more than 95% or even more than 99%, with SEQ ID NO: 877, wherein the CDRs are as defined above. In one embodiment, the ISVD specifically binding to PD-1 comprises or consists of the amino acid sequence of SEQ ID NO: 877.

In another embodiment, the ISVD specifically binding to human PD-1 may have a sequence identity of more than 90%, such as more than 95% or even more than 99%, with SEQ ID NO: 878, wherein the CDRs are as defined above. In one embodiment, the ISVD specifically binding to PD-1 comprises or consists of the amino acid sequence of SEQ ID NO: 878.

In another embodiment, the ISVD specifically binding to human PD-1 may have a sequence identity of more than 90%, such as more than 95% or even more than 99%, with SEQ ID NO: 879, wherein the CDRs are as defined above. In one embodiment, the ISVD specifically binding to PD-1 comprises or consists of the amino acid sequence of SEQ ID NO: 879.

In another embodiment, the ISVD specifically binding to human PD-1 may have a sequence identity of more than 90%, such as more than 95% or even more than 99%, with SEQ ID NO: 880, wherein the CDRs are as defined above. In one embodiment, the ISVD specifically binding to PD-1 comprises or consists of the amino acid sequence of SEQ ID NO: 880.

In another embodiment, the ISVD specifically binding to human PD-1 may have a sequence identity of more than 90%, such as more than 95% or even more than 99%, with SEQ ID NO: 881, wherein the CDRs are as defined above. In one embodiment, the ISVD specifically binding to PD-1 comprises or consists of the amino acid sequence of SEQ ID NO: 881.

In another embodiment, the ISVD specifically binding to human PD-1 may have a sequence identity of more than 90%, such as more than 95% or even more than 99%, with SEQ ID NO: 882, wherein the CDRs are as defined above. In one embodiment, the ISVD specifically binding to PD-1 comprises or consists of the amino acid sequence of SEQ ID NO: 882.

In another embodiment, the ISVD specifically binding to human PD-1 may have a sequence identity of more than 90%, such as more than 95% or even more than 99%, with SEQ ID NO: 883, wherein the CDRs are as defined above. In one embodiment, the ISVD specifically binding to PD-1 comprises or consists of the amino acid sequence of SEQ ID NO: 883.

In another embodiment, the ISVD specifically binding to human PD-1 may have a sequence identity of more than 90%, such as more than 95% or even more than 99%, with SEQ ID NO: 884, wherein the CDRs are as defined above. In one embodiment, the ISVD specifically binding to PD-1 comprises or consists of the amino acid sequence of SEQ ID NO: 884.

In another embodiment, the ISVD specifically binding to human PD-1 may have a sequence identity of more than 90%, such as more than 95% or even more than 99%, with SEQ ID NO: 885, wherein the CDRs are as defined above. In one embodiment, the ISVD specifically binding to PD-1 comprises or consists of the amino acid sequence of SEQ ID NO: 885.

In another embodiment, the ISVD specifically binding to human PD-1 may have a sequence identity of more than 90%, such as more than 95% or even more than 99%, with SEQ ID NO: 886, wherein the CDRs are as defined above. In one embodiment, the ISVD specifically binding to PD-1 comprises or consists of the amino acid sequence of SEQ ID NO: 886.

In another embodiment, the ISVD specifically binding to human PD-1 may have a sequence identity of more than 90%, such as more than 95% or even more than 99%, with SEQ ID NO: 887, wherein the CDRs are as defined above. In one embodiment, the ISVD specifically binding to PD-1 comprises or consists of the amino acid sequence of SEQ ID NO: 887.

In another embodiment, the ISVD specifically binding to human PD-1 may have a sequence identity of more than 90%, such as more than 95% or even more than 99%, with SEQ ID NO: 888, wherein the CDRs are as defined above. In one embodiment, the ISVD specifically binding to PD-1 comprises or consists of the amino acid sequence of SEQ ID NO: 888.

In another embodiment, the ISVD specifically binding to human PD-1 may have a sequence identity of more than 90%, such as more than 95% or even more than 99%, with SEQ ID NO: 889, wherein the CDRs are as defined above. In one embodiment, the ISVD specifically binding to PD-1 comprises or consists of the amino acid sequence of SEQ ID NO: 889.

In another embodiment, the ISVD specifically binding to human PD-1 may have a sequence identity of more than 90%, such as more than 95% or even more than 99%, with SEQ ID NO: 890, wherein the CDRs are as defined above. In one embodiment, the ISVD specifically binding to PD-1 comprises or consists of the amino acid sequence of SEQ ID NO: 890.

In another embodiment, the ISVD specifically binding to human PD-1 may have a sequence identity of more than 90%, such as more than 95% or even more than 99%, with SEQ ID NO: 891, wherein the CDRs are as defined above. In one embodiment, the ISVD specifically binding to PD-1 comprises or consists of the amino acid sequence of SEQ ID NO: 891.

In another embodiment, the ISVD specifically binding to human PD-1 may have a sequence identity of more than 90%, such as more than 95% or even more than 99%, with SEQ ID NO: 892, wherein the CDRs are as defined above. In one embodiment, the ISVD specifically binding to PD-1 comprises or consists of the amino acid sequence of SEQ ID NO: 892.

In another embodiment, the ISVD specifically binding to human PD-1 may have a sequence identity of more than 90%, such as more than 95% or even more than 99%, with SEQ ID NO: 893, wherein the CDRs are as defined above. In one embodiment, the ISVD specifically binding to PD-1 comprises or consists of the amino acid sequence of SEQ ID NO: 893.

In another embodiment, the ISVD specifically binding to human PD-1 may have a sequence identity of more than 90%, such as more than 95% or even more than 99%, with SEQ ID NO: 894, wherein the CDRs are as defined above. In one embodiment, the ISVD specifically binding to PD-1 comprises or consists of the amino acid sequence of SEQ ID NO: 894.

In another embodiment, the ISVD specifically binding to human PD-1 may have a sequence identity of more than 90%, such as more than 95% or even more than 99%, with SEQ ID NO: 895, wherein the CDRs are as defined above. In one embodiment, the ISVD specifically binding to PD-1 comprises or consists of the amino acid sequence of SEQ ID NO: 895.

In another embodiment, the ISVD specifically binding to human PD-1 may have a sequence identity of more than 90%, such as more than 95% or even more than 99%, with SEQ ID NO: 896, wherein the CDRs are as defined above. In one embodiment, the ISVD specifically binding to PD-1 comprises or consists of the amino acid sequence of SEQ ID NO: 896.

In another embodiment, the ISVD specifically binding to human PD-1 may have a sequence identity of more than 90%, such as more than 95% or even more than 99%, with SEQ ID NO: 897, wherein the CDRs are as defined above. In one embodiment, the ISVD specifically binding to PD-1 comprises or consists of the amino acid sequence of SEQ ID NO: 897.

In another embodiment, the ISVD specifically binding to human PD-1 may have a sequence identity of more than 90%, such as more than 95% or even more than 99%, with SEQ ID NO: 898, wherein the CDRs are as defined above. In one embodiment, the ISVD specifically binding to PD-1 comprises or consists of the amino acid sequence of SEQ ID NO: 898.

In another embodiment, the ISVD specifically binding to human PD-1 may have a sequence identity of more than 90%, such as more than 95% or even more than 99%, with SEQ ID NO: 899, wherein the CDRs are as defined above. In one embodiment, the ISVD specifically binding to PD-1 comprises or consists of the amino acid sequence of SEQ ID NO: 899.

In another embodiment, the ISVD specifically binding to human PD-1 may have a sequence identity of more than 90%, such as more than 95% or even more than 99%, with SEQ ID NO: 900, wherein the CDRs are as defined above. In one embodiment, the ISVD specifically binding to PD-1 comprises or consists of the amino acid sequence of SEQ ID NO: 900.

In another embodiment, the ISVD specifically binding to human PD-1 may have a sequence identity of more than 90%, such as more than 95% or even more than 99%, with SEQ ID NO: 901, wherein the CDRs are as defined above. In one embodiment, the ISVD specifically binding to PD-1 comprises or consists of the amino acid sequence of SEQ ID NO: 901.

In another embodiment, the ISVD specifically binding to human PD-1 may have a sequence identity of more than 90%, such as more than 95% or even more than 99%, with SEQ ID NO: 902, wherein the CDRs are as defined above. In one embodiment, the ISVD specifically binding to PD-1 comprises or consists of the amino acid sequence of SEQ ID NO: 902.

In another embodiment, the ISVD specifically binding to human PD-1 may have a sequence identity of more than 90%, such as more than 95% or even more than 99%, with SEQ ID NO: 903, wherein the CDRs are as defined above. In one embodiment, the ISVD specifically binding to PD-1 comprises or consists of the amino acid sequence of SEQ ID NO: 903.

In another embodiment, the ISVD specifically binding to human PD-1 may have a sequence identity of more than 90%, such as more than 95% or even more than 99%, with SEQ ID NO: 904, wherein the CDRs are as defined above. In one embodiment, the ISVD specifically binding to PD-1 comprises or consists of the amino acid sequence of SEQ ID NO: 904.

In another embodiment, the ISVD specifically binding to human PD-1 may have a sequence identity of more than 90%, such as more than 95% or even more than 99%, with SEQ ID NO: 905, wherein the CDRs are as defined above. In one embodiment, the ISVD specifically binding to PD-1 comprises or consists of the amino acid sequence of SEQ ID NO: 905.

In another embodiment, the ISVD specifically binding to human PD-1 may have a sequence identity of more than 90%, such as more than 95% or even more than 99%, with SEQ ID NO: 906, wherein the CDRs are as defined above. In one embodiment, the ISVD specifically binding to PD-1 comprises or consists of the amino acid sequence of SEQ ID NO: 906.

In another embodiment, the ISVD specifically binding to human PD-1 may have a sequence identity of more than 90%, such as more than 95% or even more than 99%, with SEQ ID NO: 907, wherein the CDRs are as defined above. In one embodiment, the ISVD specifically binding to PD-1 comprises or consists of the amino acid sequence of SEQ ID NO: 907.

In another embodiment, the ISVD specifically binding to human PD-1 may have a sequence identity of more than 90%, such as more than 95% or even more than 99%, with SEQ ID NO: 908, wherein the CDRs are as defined above. In one embodiment, the ISVD specifically binding to PD-1 comprises or consists of the amino acid sequence of SEQ ID NO: 908.

In another embodiment, the ISVD specifically binding to human PD-1 may have a sequence identity of more than 90%, such as more than 95% or even more than 99%, with SEQ ID NO: 909, wherein the CDRs are as defined above. In one embodiment, the ISVD specifically binding to PD-1 comprises or consists of the amino acid sequence of SEQ ID NO: 909.

In another embodiment, the ISVD specifically binding to human PD-1 may have a sequence identity of more than 90%, such as more than 95% or even more than 99%, with SEQ ID NO: 910, wherein the CDRs are as defined above. In one embodiment, the ISVD specifically binding to PD-1 comprises or consists of the amino acid sequence of SEQ ID NO: 910.

In another embodiment, the ISVD specifically binding to human PD-1 may have a sequence identity of more than 90%, such as more than 95% or even more than 99%, with SEQ ID NO: 911, wherein the CDRs are as defined above. In one embodiment, the ISVD specifically binding to PD-1 comprises or consists of the amino acid sequence of SEQ ID NO: 911.

In another embodiment, the ISVD specifically binding to human PD-1 may have a sequence identity of more than 90%, such as more than 95% or even more than 99%, with SEQ ID NO: 912, wherein the CDRs are as defined above. In one embodiment, the ISVD specifically binding to PD-1 comprises or consists of the amino acid sequence of SEQ ID NO: 912.

In another embodiment, the ISVD specifically binding to human PD-1 may have a sequence identity of more than 90%, such as more than 95% or even more than 99%, with SEQ ID NO: 913, wherein the CDRs are as defined above. In one embodiment, the ISVD specifically binding to PD-1 comprises or consists of the amino acid sequence of SEQ ID NO: 913.

In another embodiment, the ISVD specifically binding to human PD-1 may have a sequence identity of more than 90%, such as more than 95% or even more than 99%, with SEQ ID NO: 914, wherein the CDRs are as defined above. In one embodiment, the ISVD specifically binding to PD-1 comprises or consists of the amino acid sequence of SEQ ID NO: 914.

In another embodiment, the ISVD specifically binding to human PD-1 may have a sequence identity of more than 90%, such as more than 95% or even more than 99%, with SEQ ID NO: 915, wherein the CDRs are as defined above. In one embodiment, the ISVD specifically binding to PD-1 comprises or consists of the amino acid sequence of SEQ ID NO: 915.

In another embodiment, the ISVD specifically binding to human PD-1 may have a sequence identity of more than 90%, such as more than 95% or even more than 99%, with SEQ ID NO: 916, wherein the CDRs are as defined above. In one embodiment, the ISVD specifically binding to PD-1 comprises or consists of the amino acid sequence of SEQ ID NO: 916.

In another embodiment, the ISVD specifically binding to human PD-1 may have a sequence identity of more than 90%, such as more than 95% or even more than 99%, with SEQ ID NO: 917, wherein the CDRs are as defined above. In one embodiment, the ISVD specifically binding to PD-1 comprises or consists of the amino acid sequence of SEQ ID NO: 917.

In another embodiment, the ISVD specifically binding to human PD-1 may have a sequence identity of more than 90%, such as more than 95% or even more than 99%, with SEQ ID NO: 918, wherein the CDRs are as defined above. In one embodiment, the ISVD specifically binding to PD-1 comprises or consists of the amino acid sequence of SEQ ID NO: 918.

In another embodiment, the ISVD specifically binding to human PD-1 may have a sequence identity of more than 90%, such as more than 95% or even more than 99%, with SEQ ID NO: 919, wherein the CDRs are as defined above. In one embodiment, the ISVD specifically binding to PD-1 comprises or consists of the amino acid sequence of SEQ ID NO: 919.

In another embodiment, the ISVD specifically binding to human PD-1 may have a sequence identity of more than 90%, such as more than 95% or even more than 99%, with SEQ ID NO: 920, wherein the CDRs are as defined above. In one embodiment, the ISVD specifically binding to PD-1 comprises or consists of the amino acid sequence of SEQ ID NO: 920.

In another embodiment, the ISVD specifically binding to human PD-1 may have a sequence identity of more than 90%, such as more than 95% or even more than 99%, with SEQ ID NO: 921, wherein the CDRs are as defined above. In one embodiment, the ISVD specifically binding to PD-1 comprises or consists of the amino acid sequence of SEQ ID NO: 921.

In another embodiment, the ISVD specifically binding to human PD-1 may have a sequence identity of more than 90%, such as more than 95% or even more than 99%, with SEQ ID NO: 922, wherein the CDRs are as defined above. In one embodiment, the ISVD specifically binding to PD-1 comprises or consists of the amino acid sequence of SEQ ID NO: 922.

In another embodiment, the ISVD specifically binding to human PD-1 may have a sequence identity of more than 90%, such as more than 95% or even more than 99%, with SEQ ID NO: 923, wherein the CDRs are as defined above. In one embodiment, the ISVD specifically binding to PD-1 comprises or consists of the amino acid sequence of SEQ ID NO: 923.

In another embodiment, the ISVD specifically binding to human PD-1 may have a sequence identity of more than 90%, such as more than 95% or even more than 99%, with SEQ ID NO: 924, wherein the CDRs are as defined above. In one embodiment, the ISVD specifically binding to PD-1 comprises or consists of the amino acid sequence of SEQ ID NO: 924.

In another embodiment, the ISVD specifically binding to human PD-1 may have a sequence identity of more than 90%, such as more than 95% or even more than 99%, with SEQ ID NO: 925, wherein the CDRs are as defined above. In one embodiment, the ISVD specifically binding to PD-1 comprises or consists of the amino acid sequence of SEQ ID NO: 925.

In another embodiment, the ISVD specifically binding to human PD-1 may have a sequence identity of more than 90%, such as more than 95% or even more than 99%, with SEQ ID NO: 926, wherein the CDRs are as defined above. In one embodiment, the ISVD specifically binding to PD-1 comprises or consists of the amino acid sequence of SEQ ID NO: 926.

In another embodiment, the ISVD specifically binding to human PD-1 may have a sequence identity of more than 90%, such as more than 95% or even more than 99%, with SEQ ID NO: 927, wherein the CDRs are as defined above. In one embodiment, the ISVD specifically binding to PD-1 comprises or consists of the amino acid sequence of SEQ ID NO: 927.

In another embodiment, the ISVD specifically binding to human PD-1 may have a sequence identity of more than 90%, such as more than 95% or even more than 99%, with SEQ ID NO: 928, wherein the CDRs are as defined above. In one embodiment, the ISVD specifically binding to PD-1 comprises or consists of the amino acid sequence of SEQ ID NO: 928.

In another embodiment, the ISVD specifically binding to human PD-1 may have a sequence identity of more than 90%, such as more than 95% or even more than 99%, with SEQ ID NO: 929, wherein the CDRs are as defined above. In one embodiment, the ISVD specifically binding to PD-1 comprises or consists of the amino acid sequence of SEQ ID NO: 929.

In another embodiment, the ISVD specifically binding to human PD-1 may have a sequence identity of more than 90%, such as more than 95% or even more than 99%, with SEQ ID NO: 930, wherein the CDRs are as defined above. In one embodiment, the ISVD specifically binding to PD-1 comprises or consists of the amino acid sequence of SEQ ID NO: 930.

In another embodiment, the ISVD specifically binding to human PD-1 may have a sequence identity of more than 90%, such as more than 95% or even more than 99%, with SEQ ID NO: 931, wherein the CDRs are as defined above. In one embodiment, the ISVD specifically binding to PD-1 comprises or consists of the amino acid sequence of SEQ ID NO: 931.

In another embodiment, the ISVD specifically binding to human PD-1 may have a sequence identity of more than 90%, such as more than 95% or even more than 99%, with SEQ ID NO: 932, wherein the CDRs are as defined above. In one embodiment, the ISVD specifically binding to PD-1 comprises or consists of the amino acid sequence of SEQ ID NO: 932.

In another embodiment, the ISVD specifically binding to human PD-1 may have a sequence identity of more than 90%, such as more than 95% or even more than 99%, with SEQ ID NO: 933, wherein the CDRs are as defined above. In one embodiment, the ISVD specifically binding to PD-1 comprises or consists of the amino acid sequence of SEQ ID NO: 933.

In another embodiment, the ISVD specifically binding to human PD-1 may have a sequence identity of more than 90%, such as more than 95% or even more than 99%, with SEQ ID NO: 934, wherein the CDRs are as defined above. In one embodiment, the ISVD specifically binding to PD-1 comprises or consists of the amino acid sequence of SEQ ID NO: 934.

In another embodiment, the ISVD specifically binding to human PD-1 may have a sequence identity of more than 90%, such as more than 95% or even more than 99%, with SEQ ID NO: 935, wherein the CDRs are as defined above. In one embodiment, the ISVD specifically binding to PD-1 comprises or consists of the amino acid sequence of SEQ ID NO: 935.

In another embodiment, the ISVD specifically binding to human PD-1 may have a sequence identity of more than 90%, such as more than 95% or even more than 99%, with SEQ ID NO: 936, wherein the CDRs are as defined above. In one embodiment, the ISVD specifically binding to PD-1 comprises or consists of the amino acid sequence of SEQ ID NO: 936.

In another embodiment, the ISVD specifically binding to human PD-1 may have a sequence identity of more than 90%, such as more than 95% or even more than 99%, with SEQ ID NO: 937, wherein the CDRs are as defined above. In one embodiment, the ISVD specifically binding to PD-1 comprises or consists of the amino acid sequence of SEQ ID NO: 937.

In another embodiment, the ISVD specifically binding to human PD-1 may have a sequence identity of more than 90%, such as more than 95% or even more than 99%, with SEQ ID NO: 938, wherein the CDRs are as defined above. In one embodiment, the ISVD specifically binding to PD-1 comprises or consists of the amino acid sequence of SEQ ID NO: 938.

In another embodiment, the ISVD specifically binding to human PD-1 may have a sequence identity of more than 90%, such as more than 95% or even more than 99%, with SEQ ID NO: 939, wherein the CDRs are as defined above. In one embodiment, the ISVD specifically binding to PD-1 comprises or consists of the amino acid sequence of SEQ ID NO: 939.

In another embodiment, the ISVD specifically binding to human PD-1 may have a sequence identity of more than 90%, such as more than 95% or even more than 99%, with SEQ ID NO: 940, wherein the CDRs are as defined above. In one embodiment, the ISVD specifically binding to PD-1 comprises or consists of the amino acid sequence of SEQ ID NO: 940.

In another embodiment, the ISVD specifically binding to human PD-1 may have a sequence identity of more than 90%, such as more than 95% or even more than 99%, with SEQ ID NO: 941, wherein the CDRs are as defined above. In one embodiment, the ISVD specifically binding to PD-1 comprises or consists of the amino acid sequence of SEQ ID NO: 941.

In another embodiment, the ISVD specifically binding to human PD-1 may have a sequence identity of more than 90%, such as more than 95% or even more than 99%, with SEQ ID NO: 942, wherein the CDRs are as defined above. In one embodiment, the ISVD specifically binding to PD-1 comprises or consists of the amino acid sequence of SEQ ID NO: 942.

In another embodiment, the ISVD specifically binding to human PD-1 may have a sequence identity of more than 90%, such as more than 95% or even more than 99%, with SEQ ID NO: 943, wherein the CDRs are as defined above. In one embodiment, the ISVD specifically binding to PD-1 comprises or consists of the amino acid sequence of SEQ ID NO: 943.

In another embodiment, the ISVD specifically binding to human PD-1 may have a sequence identity of more than 90%, such as more than 95% or even more than 99%, with SEQ ID NO: 944, wherein the CDRs are as defined above. In one embodiment, the ISVD specifically binding to PD-1 comprises or consists of the amino acid sequence of SEQ ID NO: 944.

In another embodiment, the ISVD specifically binding to human PD-1 may have a sequence identity of more than 90%, such as more than 95% or even more than 99%, with SEQ ID NO: 945, wherein the CDRs are as defined above. In one embodiment, the ISVD specifically binding to PD-1 comprises or consists of the amino acid sequence of SEQ ID NO: 945.

In another embodiment, the ISVD specifically binding to human PD-1 may have a sequence identity of more than 90%, such as more than 95% or even more than 99%, with SEQ ID NO: 946, wherein the CDRs are as defined above. In one embodiment, the ISVD specifically binding to PD-1 comprises or consists of the amino acid sequence of SEQ ID NO: 946.

In another embodiment, the ISVD specifically binding to human PD-1 may have a sequence identity of more than 90%, such as more than 95% or even more than 99%, with SEQ ID NO: 947, wherein the CDRs are as defined above. In one embodiment, the ISVD specifically binding to PD-1 comprises or consists of the amino acid sequence of SEQ ID NO: 947.

In another embodiment, the ISVD specifically binding to human PD-1 may have a sequence identity of more than 90%, such as more than 95% or even more than 99%, with SEQ ID NO: 948, wherein the CDRs are as defined above. In one embodiment, the ISVD specifically binding to PD-1 comprises or consists of the amino acid sequence of SEQ ID NO: 948.

In another embodiment, the ISVD specifically binding to human PD-1 may have a sequence identity of more than 90%, such as more than 95% or even more than 99%, with SEQ ID NO: 949, wherein the CDRs are as defined above. In one embodiment, the ISVD specifically binding to PD-1 comprises or consists of the amino acid sequence of SEQ ID NO: 949.

In another embodiment, the ISVD specifically binding to human PD-1 may have a sequence identity of more than 90%, such as more than 95% or even more than 99%, with SEQ ID NO: 950, wherein the CDRs are as defined above. In one embodiment, the ISVD specifically binding to PD-1 comprises or consists of the amino acid sequence of SEQ ID NO: 950.

In another embodiment, the ISVD specifically binding to human PD-1 may have a sequence identity of more than 90%, such as more than 95% or even more than 99%, with SEQ ID NO: 951, wherein the CDRs are as defined above. In one embodiment, the ISVD specifically binding to PD-1 comprises or consists of the amino acid sequence of SEQ ID NO: 951.

In another embodiment, the ISVD specifically binding to human PD-1 may have a sequence identity of more than 90%, such as more than 95% or even more than 99%, with SEQ ID NO: 952, wherein the CDRs are as defined above. In one embodiment, the ISVD specifically binding to PD-1 comprises or consists of the amino acid sequence of SEQ ID NO: 952.

In another embodiment, the ISVD specifically binding to human PD-1 may have a sequence identity of more than 90%, such as more than 95% or even more than 99%, with SEQ ID NO: 953, wherein the CDRs are as defined above. In one embodiment, the ISVD specifically binding to PD-1 comprises or consists of the amino acid sequence of SEQ ID NO: 953.

In another embodiment, the ISVD specifically binding to human PD-1 may have a sequence identity of more than 90%, such as more than 95% or even more than 99%, with SEQ ID NO: 954, wherein the CDRs are as defined above. In one embodiment, the ISVD specifically binding to PD-1 comprises or consists of the amino acid sequence of SEQ ID NO: 954.

In another embodiment, the ISVD specifically binding to human PD-1 may have a sequence identity of more than 90%, such as more than 95% or even more than 99%, with SEQ ID NO: 955, wherein the CDRs are as defined above. In one embodiment, the ISVD specifically binding to PD-1 comprises or consists of the amino acid sequence of SEQ ID NO: 955.

In another embodiment, the ISVD specifically binding to human PD-1 may have a sequence identity of more than 90%, such as more than 95% or even more than 99%, with SEQ ID NO: 956, wherein the CDRs are as defined above. In one embodiment, the ISVD specifically binding to PD-1 comprises or consists of the amino acid sequence of SEQ ID NO: 956.

In another embodiment, the ISVD specifically binding to human PD-1 may have a sequence identity of more than 90%, such as more than 95% or even more than 99%, with SEQ ID NO: 957, wherein the CDRs are as defined above. In one embodiment, the ISVD specifically binding to PD-1 comprises or consists of the amino acid sequence of SEQ ID NO: 957.

In another embodiment, the ISVD specifically binding to human PD-1 may have a sequence identity of more than 90%, such as more than 95% or even more than 99%, with SEQ ID NO: 958, wherein the CDRs are as defined above. In one embodiment, the ISVD specifically binding to PD-1 comprises or consists of the amino acid sequence of SEQ ID NO: 958.

In another embodiment, the ISVD specifically binding to human PD-1 may have a sequence identity of more than 90%, such as more than 95% or even more than 99%, with SEQ ID NO: 959, wherein the CDRs are as defined above. In one embodiment, the ISVD specifically binding to PD-1 comprises or consists of the amino acid sequence of SEQ ID NO: 959.

In another embodiment, the ISVD specifically binding to human PD-1 may have a sequence identity of more than 90%, such as more than 95% or even more than 99%, with SEQ ID NO: 960, wherein the CDRs are as defined above. In one embodiment, the ISVD specifically binding to PD-1 comprises or consists of the amino acid sequence of SEQ ID NO: 960.

In another embodiment, the ISVD specifically binding to human PD-1 may have a sequence identity of more than 90%, such as more than 95% or even more than 99%, with SEQ ID NO: 961, wherein the CDRs are as defined above. In one embodiment, the ISVD specifically binding to PD-1 comprises or consists of the amino acid sequence of SEQ ID NO: 961.

In another embodiment, the ISVD specifically binding to human PD-1 may have a sequence identity of more than 90%, such as more than 95% or even more than 99%, with SEQ ID NO: 962, wherein the CDRs are as defined above. In one embodiment, the ISVD specifically binding to PD-1 comprises or consists of the amino acid sequence of SEQ ID NO: 962.

In another embodiment, the ISVD specifically binding to human PD-1 may have a sequence identity of more than 90%, such as more than 95% or even more than 99%, with SEQ ID NO: 963, wherein the CDRs are as defined above. In one embodiment, the ISVD specifically binding to PD-1 comprises or consists of the amino acid sequence of SEQ ID NO: 963.

In another embodiment, the ISVD specifically binding to human PD-1 may have a sequence identity of more than 90%, such as more than 95% or even more than 99%, with SEQ ID NO: 964, wherein the CDRs are as defined above. In one embodiment, the ISVD specifically binding to PD-1 comprises or consists of the amino acid sequence of SEQ ID NO: 964.

In another embodiment, the ISVD specifically binding to human PD-1 may have a sequence identity of more than 90%, such as more than 95% or even more than 99%, with SEQ ID NO: 965, wherein the CDRs are as defined above. In one embodiment, the ISVD specifically binding to PD-1 comprises or consists of the amino acid sequence of SEQ ID NO: 965.

In another embodiment, the ISVD specifically binding to human PD-1 may have a sequence identity of more than 90%, such as more than 95% or even more than 99%, with SEQ ID NO: 966, wherein the CDRs are as defined above. In one embodiment, the ISVD specifically binding to PD-1 comprises or consists of the amino acid sequence of SEQ ID NO: 966.

In another embodiment, the ISVD specifically binding to human PD-1 may have a sequence identity of more than 90%, such as more than 95% or even more than 99%, with SEQ ID NO: 967, wherein the CDRs are as defined above. In one embodiment, the ISVD specifically binding to PD-1 comprises or consists of the amino acid sequence of SEQ ID NO: 967.

In another embodiment, the ISVD specifically binding to human PD-1 may have a sequence identity of more than 90%, such as more than 95% or even more than 99%, with SEQ ID NO: 968, wherein the CDRs are as defined above. In one embodiment, the ISVD specifically binding to PD-1 comprises or consists of the amino acid sequence of SEQ ID NO: 968.

In another embodiment, the ISVD specifically binding to human PD-1 may have a sequence identity of more than 90%, such as more than 95% or even more than 99%, with SEQ ID NO: 969, wherein the CDRs are as defined above. In one embodiment, the ISVD specifically binding to PD-1 comprises or consists of the amino acid sequence of SEQ ID NO: 969.

In another embodiment, the ISVD specifically binding to human PD-1 may have a sequence identity of more than 90%, such as more than 95% or even more than 99%, with SEQ ID NO: 970, wherein the CDRs are as defined above. In one embodiment, the ISVD specifically binding to PD-1 comprises or consists of the amino acid sequence of SEQ ID NO: 970.

In another embodiment, the ISVD specifically binding to human PD-1 may have a sequence identity of more than 90%, such as more than 95% or even more than 99%, with SEQ ID NO: 971, wherein the CDRs are as defined above. In one embodiment, the ISVD specifically binding to PD-1 comprises or consists of the amino acid sequence of SEQ ID NO: 971.

In another embodiment, the ISVD specifically binding to human PD-1 may have a sequence identity of more than 90%, such as more than 95% or even more than 99%, with SEQ ID NO: 972, wherein the CDRs are as defined above. In one embodiment, the ISVD specifically binding to PD-1 comprises or consists of the amino acid sequence of SEQ ID NO: 972.

In another embodiment, the ISVD specifically binding to human PD-1 may have a sequence identity of more than 90%, such as more than 95% or even more than 99%, with SEQ ID NO: 973, wherein the CDRs are as defined above. In one embodiment, the ISVD specifically binding to PD-1 comprises or consists of the amino acid sequence of SEQ ID NO: 973.

In another embodiment, the ISVD specifically binding to human PD-1 may have a sequence identity of more than 90%, such as more than 95% or even more than 99%, with SEQ ID NO: 974, wherein the CDRs are as defined above. In one embodiment, the ISVD specifically binding to PD-1 comprises or consists of the amino acid sequence of SEQ ID NO: 974.

In another embodiment, the ISVD specifically binding to human PD-1 may have a sequence identity of more than 90%, such as more than 95% or even more than 99%, with SEQ ID NO: 975, wherein the CDRs are as defined above. In one embodiment, the ISVD specifically binding to PD-1 comprises or consists of the amino acid sequence of SEQ ID NO: 975.

In another embodiment, the ISVD specifically binding to human PD-1 may have a sequence identity of more than 90%, such as more than 95% or even more than 99%, with SEQ ID NO: 976, wherein the CDRs are as defined above. In one embodiment, the ISVD specifically binding to PD-1 comprises or consists of the amino acid sequence of SEQ ID NO: 976.

In another embodiment, the ISVD specifically binding to human PD-1 may have a sequence identity of more than 90%, such as more than 95% or even more than 99%, with SEQ ID NO: 977, wherein the CDRs are as defined above. In one embodiment, the ISVD specifically binding to PD-1 comprises or consists of the amino acid sequence of SEQ ID NO: 977.

In another embodiment, the ISVD specifically binding to human PD-1 may have a sequence identity of more than 90%, such as more than 95% or even more than 99%, with SEQ ID NO: 978, wherein the CDRs are as defined above. In one embodiment, the ISVD specifically binding to PD-1 comprises or consists of the amino acid sequence of SEQ ID NO: 978.

In another embodiment, the ISVD specifically binding to human PD-1 may have a sequence identity of more than 90%, such as more than 95% or even more than 99%, with SEQ ID NO: 979, wherein the CDRs are as defined above. In one embodiment, the ISVD specifically binding to PD-1 comprises or consists of the amino acid sequence of SEQ ID NO: 979.

In another embodiment, the ISVD specifically binding to human PD-1 may have a sequence identity of more than 90%, such as more than 95% or even more than 99%, with SEQ ID NO: 980, wherein the CDRs are as defined above. In one embodiment, the ISVD specifically binding to PD-1 comprises or consists of the amino acid sequence of SEQ ID NO: 980.

In another embodiment, the ISVD specifically binding to human PD-1 may have a sequence identity of more than 90%, such as more than 95% or even more than 99%, with SEQ ID NO: 981, wherein the CDRs are as defined above. In one embodiment, the ISVD specifically binding to PD-1 comprises or consists of the amino acid sequence of SEQ ID NO: 981.

In another embodiment, the ISVD specifically binding to human PD-1 may have a sequence identity of more than 90%, such as more than 95% or even more than 99%, with SEQ ID NO: 982, wherein the CDRs are as defined above. In one embodiment, the ISVD specifically binding to PD-1 comprises or consists of the amino acid sequence of SEQ ID NO: 982.

In another embodiment, the ISVD specifically binding to human PD-1 may have a sequence identity of more than 90%, such as more than 95% or even more than 99%, with SEQ ID NO: 983, wherein the CDRs are as defined above. In one embodiment, the ISVD specifically binding to PD-1 comprises or consists of the amino acid sequence of SEQ ID NO: 983.

In another embodiment, the ISVD specifically binding to human PD-1 may have a sequence identity of more than 90%, such as more than 95% or even more than 99%, with SEQ ID NO: 984, wherein the CDRs are as defined above. In one embodiment, the ISVD specifically binding to PD-1 comprises or consists of the amino acid sequence of SEQ ID NO: 984.

In another embodiment, the ISVD specifically binding to human PD-1 may have a sequence identity of more than 90%, such as more than 95% or even more than 99%, with SEQ ID NO: 985, wherein the CDRs are as defined above. In one embodiment, the ISVD specifically binding to PD-1 comprises or consists of the amino acid sequence of SEQ ID NO: 985.

In another embodiment, the ISVD specifically binding to human PD-1 may have a sequence identity of more than 90%, such as more than 95% or even more than 99%, with SEQ ID NO: 986, wherein the CDRs are as defined above. In one embodiment, the ISVD specifically binding to PD-1 comprises or consists of the amino acid sequence of SEQ ID NO: 986.

In another embodiment, the ISVD specifically binding to human PD-1 may have a sequence identity of more than 90%, such as more than 95% or even more than 99%, with SEQ ID NO: 987, wherein the CDRs are as defined above. In one embodiment, the ISVD specifically binding to PD-1 comprises or consists of the amino acid sequence of SEQ ID NO: 987.

In another embodiment, the ISVD specifically binding to human PD-1 may have a sequence identity of more than 90%, such as more than 95% or even more than 99%, with SEQ ID NO: 988, wherein the CDRs are as defined above. In one embodiment, the ISVD specifically binding to PD-1 comprises or consists of the amino acid sequence of SEQ ID NO: 988.

In another embodiment, the ISVD specifically binding to human PD-1 may have a sequence identity of more than 90%, such as more than 95% or even more than 99%, with SEQ ID NO: 989, wherein the CDRs are as defined above. In one embodiment, the ISVD specifically binding to PD-1 comprises or consists of the amino acid sequence of SEQ ID NO: 989.

In another embodiment, the ISVD specifically binding to human PD-1 may have a sequence identity of more than 90%, such as more than 95% or even more than 99%, with SEQ ID NO: 990, wherein the CDRs are as defined above. In one embodiment, the ISVD specifically binding to PD-1 comprises or consists of the amino acid sequence of SEQ ID NO: 990.

In another embodiment, the ISVD specifically binding to human PD-1 may have a sequence identity of more than 90%, such as more than 95% or even more than 99%, with SEQ ID NO: 991, wherein the CDRs are as defined above. In one embodiment, the ISVD specifically binding to PD-1 comprises or consists of the amino acid sequence of SEQ ID NO: 991.

In another embodiment, the ISVD specifically binding to human PD-1 may have a sequence identity of more than 90%, such as more than 95% or even more than 99%, with SEQ ID NO: 992, wherein the CDRs are as defined above. In one embodiment, the ISVD specifically binding to PD-1 comprises or consists of the amino acid sequence of SEQ ID NO: 992.

In another embodiment, the ISVD specifically binding to human PD-1 may have a sequence identity of more than 90%, such as more than 95% or even more than 99%, with SEQ ID NO: 993, wherein the CDRs are as defined above. In one embodiment, the ISVD specifically binding to PD-1 comprises or consists of the amino acid sequence of SEQ ID NO: 993.

In another embodiment, the ISVD specifically binding to human PD-1 may have a sequence identity of more than 90%, such as more than 95% or even more than 99%, with SEQ ID NO: 994, wherein the CDRs are as defined above. In one embodiment, the ISVD specifically binding to PD-1 comprises or consists of the amino acid sequence of SEQ ID NO: 994.

In another embodiment, the ISVD specifically binding to human PD-1 may have a sequence identity of more than 90%, such as more than 95% or even more than 99%, with SEQ ID NO: 995, wherein the CDRs are as defined above. In one embodiment, the ISVD specifically binding to PD-1 comprises or consists of the amino acid sequence of SEQ ID NO: 995.

In another embodiment, the ISVD specifically binding to human PD-1 may have a sequence identity of more than 90%, such as more than 95% or even more than 99%, with SEQ ID NO: 996, wherein the CDRs are as defined above. In one embodiment, the ISVD specifically binding to PD-1 comprises or consists of the amino acid sequence of SEQ ID NO: 996.

In another embodiment, the ISVD specifically binding to human PD-1 may have a sequence identity of more than 90%, such as more than 95% or even more than 99%, with SEQ ID NO: 997, wherein the CDRs are as defined above. In one embodiment, the ISVD specifically binding to PD-1 comprises or consists of the amino acid sequence of SEQ ID NO: 997.

In another embodiment, the ISVD specifically binding to human PD-1 may have a sequence identity of more than 90%, such as more than 95% or even more than 99%, with SEQ ID NO: 998, wherein the CDRs are as defined above. In one embodiment, the ISVD specifically binding to PD-1 comprises or consists of the amino acid sequence of SEQ ID NO: 998.

In another embodiment, the ISVD specifically binding to human PD-1 may have a sequence identity of more than 90%, such as more than 95% or even more than 99%, with SEQ ID NO: 999, wherein the CDRs are as defined above. In one embodiment, the ISVD specifically binding to PD-1 comprises or consists of the amino acid sequence of SEQ ID NO: 999.

In another embodiment, the ISVD specifically binding to human PD-1 may have a sequence identity of more than 90%, such as more than 95% or even more than 99%, with SEQ ID NO: 1000, wherein the CDRs are as defined above. In one embodiment, the ISVD specifically binding to PD-1 comprises or consists of the amino acid sequence of SEQ ID NO: 1000.

In another embodiment, the ISVD specifically binding to human PD-1 may have a sequence identity of more than 90%, such as more than 95% or even more than 99%, with SEQ ID NO: 1001, wherein the CDRs are as defined above. In one embodiment, the ISVD specifically binding to PD-1 comprises or consists of the amino acid sequence of SEQ ID NO: 1001.

In another embodiment, the ISVD specifically binding to human PD-1 may have a sequence identity of more than 90%, such as more than 95% or even more than 99%, with SEQ ID NO: 1002, wherein the CDRs are as defined above. In one embodiment, the ISVD specifically binding to PD-1 comprises or consists of the amino acid sequence of SEQ ID NO: 1002.

In another embodiment, the ISVD specifically binding to human PD-1 may have a sequence identity of more than 90%, such as more than 95% or even more than 99%, with SEQ ID NO: 1003, wherein the CDRs are as defined above. In one embodiment, the ISVD specifically binding to PD-1 comprises or consists of the amino acid sequence of SEQ ID NO: 1003.

In another embodiment, the ISVD specifically binding to human PD-1 may have a sequence identity of more than 90%, such as more than 95% or even more than 99%, with SEQ ID NO: 1004, wherein the CDRs are as defined above. In one embodiment, the ISVD specifically binding to PD-1 comprises or consists of the amino acid sequence of SEQ ID NO: 1004.

In another embodiment, the ISVD specifically binding to human PD-1 may have a sequence identity of more than 90%, such as more than 95% or even more than 99%, with SEQ ID NO: 1005, wherein the CDRs are as defined above. In one embodiment, the ISVD specifically binding to PD-1 comprises or consists of the amino acid sequence of SEQ ID NO: 1005.

In another embodiment, the ISVD specifically binding to human PD-1 may have a sequence identity of more than 90%, such as more than 95% or even more than 99%, with SEQ ID NO: 1006, wherein the CDRs are as defined above. In one embodiment, the ISVD specifically binding to PD-1 comprises or consists of the amino acid sequence of SEQ ID NO: 1006.

In another embodiment, the ISVD specifically binding to human PD-1 may have a sequence identity of more than 90%, such as more than 95% or even more than 99%, with SEQ ID NO: 1007, wherein the CDRs are as defined above. In one embodiment, the ISVD specifically binding to PD-1 comprises or consists of the amino acid sequence of SEQ ID NO: 1007.

In another embodiment, the ISVD specifically binding to human PD-1 may have a sequence identity of more than 90%, such as more than 95% or even more than 99%, with SEQ ID NO: 1008, wherein the CDRs are as defined above. In one embodiment, the ISVD specifically binding to PD-1 comprises or consists of the amino acid sequence of SEQ ID NO: 1008.

In another embodiment, the ISVD specifically binding to human PD-1 may have a sequence identity of more than 90%, such as more than 95% or even more than 99%, with SEQ ID NO: 1009, wherein the CDRs are as defined above. In one embodiment, the ISVD specifically binding to PD-1 comprises or consists of the amino acid sequence of SEQ ID NO: 1009.

In another embodiment, the ISVD specifically binding to human PD-1 may have a sequence identity of more than 90%, such as more than 95% or even more than 99%, with SEQ ID NO: 1010, wherein the CDRs are as defined above. In one embodiment, the ISVD specifically binding to PD-1 comprises or consists of the amino acid sequence of SEQ ID NO: 1010.

In another embodiment, the ISVD specifically binding to human PD-1 may have a sequence identity of more than 90%, such as more than 95% or even more than 99%, with SEQ ID NO: 1011, wherein the CDRs are as defined above. In one embodiment, the ISVD specifically binding to PD-1 comprises or consists of the amino acid sequence of SEQ ID NO: 1011.

In another embodiment, the ISVD specifically binding to human PD-1 may have a sequence identity of more than 90%, such as more than 95% or even more than 99%, with SEQ ID NO: 1012, wherein the CDRs are as defined above. In one embodiment, the ISVD specifically binding to PD-1 comprises or consists of the amino acid sequence of SEQ ID NO: 1012.

In another embodiment, the ISVD specifically binding to human PD-1 may have a sequence identity of more than 90%, such as more than 95% or even more than 99%, with SEQ ID NO: 1013, wherein the CDRs are as defined above. In one embodiment, the ISVD specifically binding to PD-1 comprises or consists of the amino acid sequence of SEQ ID NO: 1013.

In another embodiment, the ISVD specifically binding to human PD-1 may have a sequence identity of more than 90%, such as more than 95% or even more than 99%, with SEQ ID NO: 1014, wherein the CDRs are as defined above. In one embodiment, the ISVD specifically binding to PD-1 comprises or consists of the amino acid sequence of SEQ ID NO: 1014.

In another embodiment, the ISVD specifically binding to human PD-1 may have a sequence identity of more than 90%, such as more than 95% or even more than 99%, with SEQ ID NO: 1015, wherein the CDRs are as defined above. In one embodiment, the ISVD specifically binding to PD-1 comprises or consists of the amino acid sequence of SEQ ID NO: 1015.

In another embodiment, the ISVD specifically binding to human PD-1 may have a sequence identity of more than 90%, such as more than 95% or even more than 99%, with SEQ ID NO: 1016, wherein the CDRs are as defined above. In one embodiment, the ISVD specifically binding to PD-1 comprises or consists of the amino acid sequence of SEQ ID NO: 1016.

In another embodiment, the ISVD specifically binding to human PD-1 may have a sequence identity of more than 90%, such as more than 95% or even more than 99%, with SEQ ID NO: 1017, wherein the CDRs are as defined above. In one embodiment, the ISVD specifically binding to PD-1 comprises or consists of the amino acid sequence of SEQ ID NO: 1017.

In another embodiment, the ISVD specifically binding to human PD-1 may have a sequence identity of more than 90%, such as more than 95% or even more than 99%, with SEQ ID NO: 1018, wherein the CDRs are as defined above. In one embodiment, the ISVD specifically binding to PD-1 comprises or consists of the amino acid sequence of SEQ ID NO: 1018.

In another embodiment, the ISVD specifically binding to human PD-1 may have a sequence identity of more than 90%, such as more than 95% or even more than 99%, with SEQ ID NO: 1019, wherein the CDRs are as defined above. In one embodiment, the ISVD specifically binding to PD-1 comprises or consists of the amino acid sequence of SEQ ID NO: 1019.

In another embodiment, the ISVD specifically binding to human PD-1 may have a sequence identity of more than 90%, such as more than 95% or even more than 99%, with SEQ ID NO: 1020, wherein the CDRs are as defined above. In one embodiment, the ISVD specifically binding to PD-1 comprises or consists of the amino acid sequence of SEQ ID NO: 1020.

In another embodiment, the ISVD specifically binding to human PD-1 may have a sequence identity of more than 90%, such as more than 95% or even more than 99%, with SEQ ID NO: 1021, wherein the CDRs are as defined above. In one embodiment, the ISVD specifically binding to PD-1 comprises or consists of the amino acid sequence of SEQ ID NO: 1021.

In another embodiment, the ISVD specifically binding to human PD-1 may have a sequence identity of more than 90%, such as more than 95% or even more than 99%, with SEQ ID NO: 1022, wherein the CDRs are as defined above. In one embodiment, the ISVD specifically binding to PD-1 comprises or consists of the amino acid sequence of SEQ ID NO: 1022.

In another embodiment, the ISVD specifically binding to human PD-1 may have a sequence identity of more than 90%, such as more than 95% or even more than 99%, with SEQ ID NO: 1023, wherein the CDRs are as defined above. In one embodiment, the ISVD specifically binding to PD-1 comprises or consists of the amino acid sequence of SEQ ID NO: 1023.

In another embodiment, the ISVD specifically binding to human PD-1 may have a sequence identity of more than 90%, such as more than 95% or even more than 99%, with SEQ ID NO: 1024, wherein the CDRs are as defined above. In one embodiment, the ISVD specifically binding to PD-1 comprises or consists of the amino acid sequence of SEQ ID NO: 1024.

In another embodiment, the ISVD specifically binding to human PD-1 may have a sequence identity of more than 90%, such as more than 95% or even more than 99%, with SEQ ID NO: 1025, wherein the CDRs are as defined above. In one embodiment, the ISVD specifically binding to PD-1 comprises or consists of the amino acid sequence of SEQ ID NO: 1025.

In another embodiment, the ISVD specifically binding to human PD-1 may have a sequence identity of more than 90%, such as more than 95% or even more than 99%, with SEQ ID NO: 1026, wherein the CDRs are as defined above. In one embodiment, the ISVD specifically binding to PD-1 comprises or consists of the amino acid sequence of SEQ ID NO: 1026.

In another embodiment, the ISVD specifically binding to human PD-1 may have a sequence identity of more than 90%, such as more than 95% or even more than 99%, with SEQ ID NO: 1027, wherein the CDRs are as defined above. In one embodiment, the ISVD specifically binding to PD-1 comprises or consists of the amino acid sequence of SEQ ID NO: 1027.

In another embodiment, the ISVD specifically binding to human PD-1 may have a sequence identity of more than 90%, such as more than 95% or even more than 99%, with SEQ ID NO: 1028, wherein the CDRs are as defined above. In one embodiment, the ISVD specifically binding to PD-1 comprises or consists of the amino acid sequence of SEQ ID NO: 1028.

In another embodiment, the ISVD specifically binding to human PD-1 may have a sequence identity of more than 90%, such as more than 95% or even more than 99%, with SEQ ID NO: 1029, wherein the CDRs are as defined above. In one embodiment, the ISVD specifically binding to PD-1 comprises or consists of the amino acid sequence of SEQ ID NO: 1029.

In another embodiment, the ISVD specifically binding to human PD-1 may have a sequence identity of more than 90%, such as more than 95% or even more than 99%, with SEQ ID NO: 1030, wherein the CDRs are as defined above. In one embodiment, the ISVD specifically binding to PD-1 comprises or consists of the amino acid sequence of SEQ ID NO: 1030.

In another embodiment, the ISVD specifically binding to human PD-1 may have a sequence identity of more than 90%, such as more than 95% or even more than 99%, with SEQ ID NO: 1031, wherein the CDRs are as defined above. In one embodiment, the ISVD specifically binding to PD-1 comprises or consists of the amino acid sequence of SEQ ID NO: 1031.

In another embodiment, the ISVD specifically binding to human PD-1 may have a sequence identity of more than 90%, such as more than 95% or even more than 99%, with SEQ ID NO: 1032, wherein the CDRs are as defined above. In one embodiment, the ISVD specifically binding to PD-1 comprises or consists of the amino acid sequence of SEQ ID NO: 1032.

In another embodiment, the ISVD specifically binding to human PD-1 may have a sequence identity of more than 90%, such as more than 95% or even more than 99%, with SEQ ID NO: 1033, wherein the CDRs are as defined above. In one embodiment, the ISVD specifically binding to PD-1 comprises or consists of the amino acid sequence of SEQ ID NO: 1033.

In another embodiment, the ISVD specifically binding to human PD-1 may have a sequence identity of more than 90%, such as more than 95% or even more than 99%, with SEQ ID NO: 1034, wherein the CDRs are as defined above. In one embodiment, the ISVD specifically binding to PD-1 comprises or consists of the amino acid sequence of SEQ ID NO: 1034.

In another embodiment, the ISVD specifically binding to human PD-1 may have a sequence identity of more than 90%, such as more than 95% or even more than 99%, with SEQ ID NO: 1035, wherein the CDRs are as defined above. In one embodiment, the ISVD specifically binding to PD-1 comprises or consists of the amino acid sequence of SEQ ID NO: 1035.

In another embodiment, the ISVD specifically binding to human PD-1 may have a sequence identity of more than 90%, such as more than 95% or even more than 99%, with SEQ ID NO: 1036, wherein the CDRs are as defined above. In one embodiment, the ISVD specifically binding to PD-1 comprises or consists of the amino acid sequence of SEQ ID NO: 1036.

In another embodiment, the ISVD specifically binding to human PD-1 may have a sequence identity of more than 90%, such as more than 95% or even more than 99%, with SEQ ID NO: 1037, wherein the CDRs are as defined above. In one embodiment, the ISVD specifically binding to PD-1 comprises or consists of the amino acid sequence of SEQ ID NO: 1037.

In another embodiment, the ISVD specifically binding to human PD-1 may have a sequence identity of more than 90%, such as more than 95% or even more than 99%, with SEQ ID NO: 1038, wherein the CDRs are as defined above. In one embodiment, the ISVD specifically binding to PD-1 comprises or consists of the amino acid sequence of SEQ ID NO: 1038.

In another embodiment, the ISVD specifically binding to human PD-1 may have a sequence identity of more than 90%, such as more than 95% or even more than 99%, with SEQ ID NO: 1039, wherein the CDRs are as defined above. In one embodiment, the ISVD specifically binding to PD-1 comprises or consists of the amino acid sequence of SEQ ID NO: 1039.

In another embodiment, the ISVD specifically binding to human PD-1 may have a sequence identity of more than 90%, such as more than 95% or even more than 99%, with SEQ ID NO: 1040, wherein the CDRs are as defined above. In one embodiment, the ISVD specifically binding to PD-1 comprises or consists of the amino acid sequence of SEQ ID NO: 1040.

In another embodiment, the ISVD specifically binding to human PD-1 may have a sequence identity of more than 90%, such as more than 95% or even more than 99%, with SEQ ID NO: 1041, wherein the CDRs are as defined above. In one embodiment, the ISVD specifically binding to PD-1 comprises or consists of the amino acid sequence of SEQ ID NO: 1041.

In another embodiment, the ISVD specifically binding to human PD-1 may have a sequence identity of more than 90%, such as more than 95% or even more than 99%, with SEQ ID NO: 1042, wherein the CDRs are as defined above. In one embodiment, the ISVD specifically binding to PD-1 comprises or consists of the amino acid sequence of SEQ ID NO: 1042.

In another embodiment, the ISVD specifically binding to human PD-1 may have a sequence identity of more than 90%, such as more than 95% or even more than 99%, with SEQ ID NO: 1043, wherein the CDRs are as defined above. In one embodiment, the ISVD specifically binding to PD-1 comprises or consists of the amino acid sequence of SEQ ID NO: 1043.

In another embodiment, the ISVD specifically binding to human PD-1 may have a sequence identity of more than 90%, such as more than 95% or even more than 99%, with SEQ ID NO: 1044, wherein the CDRs are as defined above. In one embodiment, the ISVD specifically binding to PD-1 comprises or consists of the amino acid sequence of SEQ ID NO: 1044.

In another embodiment, the ISVD specifically binding to human PD-1 may have a sequence identity of more than 90%, such as more than 95% or even more than 99%, with SEQ ID NO: 1045, wherein the CDRs are as defined above. In one embodiment, the ISVD specifically binding to PD-1 comprises or consists of the amino acid sequence of SEQ ID NO: 1045.

In another embodiment, the ISVD specifically binding to human PD-1 may have a sequence identity of more than 90%, such as more than 95% or even more than 99%, with SEQ ID NO: 1046, wherein the CDRs are as defined above. In one embodiment, the ISVD specifically binding to PD-1 comprises or consists of the amino acid sequence of SEQ ID NO: 1046.

In another embodiment, the ISVD specifically binding to human PD-1 may have a sequence identity of more than 90%, such as more than 95% or even more than 99%, with SEQ ID NO: 1047, wherein the CDRs are as defined above. In one embodiment, the ISVD specifically binding to PD-1 comprises or consists of the amino acid sequence of SEQ ID NO: 1047.

In another embodiment, the ISVD specifically binding to human PD-1 may have a sequence identity of more than 90%, such as more than 95% or even more than 99%, with SEQ ID NO: 1048, wherein the CDRs are as defined above. In one embodiment, the ISVD specifically binding to PD-1 comprises or consists of the amino acid sequence of SEQ ID NO: 1048.

In another embodiment, the ISVD specifically binding to human PD-1 may have a sequence identity of more than 90%, such as more than 95% or even more than 99%, with SEQ ID NO: 1049, wherein the CDRs are as defined above. In one embodiment, the ISVD specifically binding to PD-1 comprises or consists of the amino acid sequence of SEQ ID NO: 1049.

In another embodiment, the ISVD specifically binding to human PD-1 may have a sequence identity of more than 90%, such as more than 95% or even more than 99%, with SEQ ID NO: 1050, wherein the CDRs are as defined above. In one embodiment, the ISVD specifically binding to PD-1 comprises or consists of the amino acid sequence of SEQ ID NO: 1050.

In another embodiment, the ISVD specifically binding to human PD-1 may have a sequence identity of more than 90%, such as more than 95% or even more than 99%, with SEQ ID NO: 1051, wherein the CDRs are as defined above. In one embodiment, the ISVD specifically binding to PD-1 comprises or consists of the amino acid sequence of SEQ ID NO: 1051.

In another embodiment, the ISVD specifically binding to human PD-1 may have a sequence identity of more than 90%, such as more than 95% or even more than 99%, with SEQ ID NO: 1052, wherein the CDRs are as defined above. In one embodiment, the ISVD specifically binding to PD-1 comprises or consists of the amino acid sequence of SEQ ID NO: 1052.

In another embodiment, the ISVD specifically binding to human PD-1 may have a sequence identity of more than 90%, such as more than 95% or even more than 99%, with SEQ ID NO: 1053, wherein the CDRs are as defined above. In one embodiment, the ISVD specifically binding to PD-1 comprises or consists of the amino acid sequence of SEQ ID NO: 1053.

In another embodiment, the ISVD specifically binding to human PD-1 may have a sequence identity of more than 90%, such as more than 95% or even more than 99%, with SEQ ID NO: 1054, wherein the CDRs are as defined above. In one embodiment, the ISVD specifically binding to PD-1 comprises or consists of the amino acid sequence of SEQ ID NO: 1054.

In another embodiment, the ISVD specifically binding to human PD-1 may have a sequence identity of more than 90%, such as more than 95% or even more than 99%, with SEQ ID NO: 1055, wherein the CDRs are as defined above. In one embodiment, the ISVD specifically binding to PD-1 comprises or consists of the amino acid sequence of SEQ ID NO: 1055.

In another embodiment, the ISVD specifically binding to human PD-1 may have a sequence identity of more than 90%, such as more than 95% or even more than 99%, with SEQ ID NO: 1056, wherein the CDRs are as defined above. In one embodiment, the ISVD specifically binding to PD-1 comprises or consists of the amino acid sequence of SEQ ID NO: 1056.

In another embodiment, the ISVD specifically binding to human PD-1 may have a sequence identity of more than 90%, such as more than 95% or even more than 99%, with SEQ ID NO: 1057, wherein the CDRs are as defined above. In one embodiment, the ISVD specifically binding to PD-1 comprises or consists of the amino acid sequence of SEQ ID NO: 1057.

In another embodiment, the ISVD specifically binding to human PD-1 may have a sequence identity of more than 90%, such as more than 95% or even more than 99%, with SEQ ID NO: 1058, wherein the CDRs are as defined above. In one embodiment, the ISVD specifically binding to PD-1 comprises or consists of the amino acid sequence of SEQ ID NO: 1058.

In another embodiment, the ISVD specifically binding to human PD-1 may have a sequence identity of more than 90%, such as more than 95% or even more than 99%, with SEQ ID NO: 1059, wherein the CDRs are as defined above. In one embodiment, the ISVD specifically binding to PD-1 comprises or consists of the amino acid sequence of SEQ ID NO: 1059.

In another embodiment, the ISVD specifically binding to human PD-1 may have a sequence identity of more than 90%, such as more than 95% or even more than 99%, with SEQ ID NO: 1060, wherein the CDRs are as defined above. In one embodiment, the ISVD specifically binding to PD-1 comprises or consists of the amino acid sequence of SEQ ID NO: 1060.

In another embodiment, the ISVD specifically binding to human PD-1 may have a sequence identity of more than 90%, such as more than 95% or even more than 99%, with SEQ ID NO: 1061, wherein the CDRs are as defined above. In one embodiment, the ISVD specifically binding to PD-1 comprises or consists of the amino acid sequence of SEQ ID NO: 1061.

In another embodiment, the ISVD specifically binding to human PD-1 may have a sequence identity of more than 90%, such as more than 95% or even more than 99%, with SEQ ID NO: 1062, wherein the CDRs are as defined above. In one embodiment, the ISVD specifically binding to PD-1 comprises or consists of the amino acid sequence of SEQ ID NO: 1062.

In another embodiment, the ISVD specifically binding to human PD-1 may have a sequence identity of more than 90%, such as more than 95% or even more than 99%, with SEQ ID NO: 1063, wherein the CDRs are as defined above. In one embodiment, the ISVD specifically binding to PD-1 comprises or consists of the amino acid sequence of SEQ ID NO: 1063.

In another embodiment, the ISVD specifically binding to human PD-1 may have a sequence identity of more than 90%, such as more than 95% or even more than 99%, with SEQ ID NO: 1064, wherein the CDRs are as defined above. In one embodiment, the ISVD specifically binding to PD-1 comprises or consists of the amino acid sequence of SEQ ID NO: 1064.

In another embodiment, the ISVD specifically binding to human PD-1 may have a sequence identity of more than 90%, such as more than 95% or even more than 99%, with SEQ ID NO: 1065, wherein the CDRs are as defined above. In one embodiment, the ISVD specifically binding to PD-1 comprises or consists of the amino acid sequence of SEQ ID NO: 1065.

In another embodiment, the ISVD specifically binding to human PD-1 may have a sequence identity of more than 90%, such as more than 95% or even more than 99%, with SEQ ID NO: 1066, wherein the CDRs are as defined above. In one embodiment, the ISVD specifically binding to PD-1 comprises or consists of the amino acid sequence of SEQ ID NO: 1066.

In another embodiment, the ISVD specifically binding to human PD-1 may have a sequence identity of more than 90%, such as more than 95% or even more than 99%, with SEQ ID NO: 1067, wherein the CDRs are as defined above. In one embodiment, the ISVD specifically binding to PD-1 comprises or consists of the amino acid sequence of SEQ ID NO: 1067.

In another embodiment, the ISVD specifically binding to human PD-1 may have a sequence identity of more than 90%, such as more than 95% or even more than 99%, with SEQ ID NO: 1068, wherein the CDRs are as defined above. In one embodiment, the ISVD specifically binding to PD-1 comprises or consists of the amino acid sequence of SEQ ID NO: 1068.

In another embodiment, the ISVD specifically binding to human PD-1 may have a sequence identity of more than 90%, such as more than 95% or even more than 99%, with SEQ ID NO: 1069, wherein the CDRs are as defined above. In one embodiment, the ISVD specifically binding to PD-1 comprises or consists of the amino acid sequence of SEQ ID NO: 1069.

In another embodiment, the ISVD specifically binding to human PD-1 may have a sequence identity of more than 90%, such as more than 95% or even more than 99%, with SEQ ID NO: 1070, wherein the CDRs are as defined above. In one embodiment, the ISVD specifically binding to PD-1 comprises or consists of the amino acid sequence of SEQ ID NO: 1070.

In another embodiment, the ISVD specifically binding to human PD-1 may have a sequence identity of more than 90%, such as more than 95% or even more than 99%, with SEQ ID NO: 1071, wherein the CDRs are as defined above. In one embodiment, the ISVD specifically binding to PD-1 comprises or consists of the amino acid sequence of SEQ ID NO: 1071.

In another embodiment, the ISVD specifically binding to human PD-1 may have a sequence identity of more than 90%, such as more than 95% or even more than 99%, with SEQ ID NO: 1072, wherein the CDRs are as defined above. In one embodiment, the ISVD specifically binding to PD-1 comprises or consists of the amino acid sequence of SEQ ID NO: 1072.

In another embodiment, the ISVD specifically binding to human PD-1 may have a sequence identity of more than 90%, such as more than 95% or even more than 99%, with SEQ ID NO: 1073, wherein the CDRs are as defined above. In one embodiment, the ISVD specifically binding to PD-1 comprises or consists of the amino acid sequence of SEQ ID NO: 1073.

In another embodiment, the ISVD specifically binding to human PD-1 may have a sequence identity of more than 90%, such as more than 95% or even more than 99%, with SEQ ID NO: 1074, wherein the CDRs are as defined above. In one embodiment, the ISVD specifically binding to PD-1 comprises or consists of the amino acid sequence of SEQ ID NO: 1074.

In another embodiment, the ISVD specifically binding to human PD-1 may have a sequence identity of more than 90%, such as more than 95% or even more than 99%, with SEQ ID NO: 1075, wherein the CDRs are as defined above. In one embodiment, the ISVD specifically binding to PD-1 comprises or consists of the amino acid sequence of SEQ ID NO: 1075.

In another embodiment, the ISVD specifically binding to human PD-1 may have a sequence identity of more than 90%, such as more than 95% or even more than 99%, with SEQ ID NO: 1076, wherein the CDRs are as defined above. In one embodiment, the ISVD specifically binding to PD-1 comprises or consists of the amino acid sequence of SEQ ID NO: 1076.

In another embodiment, the ISVD specifically binding to human PD-1 may have a sequence identity of more than 90%, such as more than 95% or even more than 99%, with SEQ ID NO: 1077, wherein the CDRs are as defined above. In one embodiment, the ISVD specifically binding to PD-1 comprises or consists of the amino acid sequence of SEQ ID NO: 1077.

In another embodiment, the ISVD specifically binding to human PD-1 may have a sequence identity of more than 90%, such as more than 95% or even more than 99%, with SEQ ID NO: 1078, wherein the CDRs are as defined above. In one embodiment, the ISVD specifically binding to PD-1 comprises or consists of the amino acid sequence of SEQ ID NO: 1078.

In another embodiment, the ISVD specifically binding to human PD-1 may have a sequence identity of more than 90%, such as more than 95% or even more than 99%, with SEQ ID NO: 1079, wherein the CDRs are as defined above. In one embodiment, the ISVD specifically binding to PD-1 comprises or consists of the amino acid sequence of SEQ ID NO: 1079.

In another embodiment, the ISVD specifically binding to human PD-1 may have a sequence identity of more than 90%, such as more than 95% or even more than 99%, with SEQ ID NO: 1080, wherein the CDRs are as defined above. In one embodiment, the ISVD specifically binding to PD-1 comprises or consists of the amino acid sequence of SEQ ID NO: 1080.

In another embodiment, the ISVD specifically binding to human PD-1 may have a sequence identity of more than 90%, such as more than 95% or even more than 99%, with SEQ ID NO: 1081, wherein the CDRs are as defined above. In one embodiment, the ISVD specifically binding to PD-1 comprises or consists of the amino acid sequence of SEQ ID NO: 1081.

In another embodiment, the ISVD specifically binding to human PD-1 may have a sequence identity of more than 90%, such as more than 95% or even more than 99%, with SEQ ID NO: 1082, wherein the CDRs are as defined above. In one embodiment, the ISVD specifically binding to PD-1 comprises or consists of the amino acid sequence of SEQ ID NO: 1082.

In another embodiment, the ISVD specifically binding to human PD-1 may have a sequence identity of more than 90%, such as more than 95% or even more than 99%, with SEQ ID NO: 1083, wherein the CDRs are as defined above. In one embodiment, the ISVD specifically binding to PD-1 comprises or consists of the amino acid sequence of SEQ ID NO: 1083.

In another embodiment, the ISVD specifically binding to human PD-1 may have a sequence identity of more than 90%, such as more than 95% or even more than 99%, with SEQ ID NO: 1084, wherein the CDRs are as defined above. In one embodiment, the ISVD specifically binding to PD-1 comprises or consists of the amino acid sequence of SEQ ID NO: 1084.

In another embodiment, the ISVD specifically binding to human PD-1 may have a sequence identity of more than 90%, such as more than 95% or even more than 99%, with SEQ ID NO: 1085, wherein the CDRs are as defined above. In one embodiment, the ISVD specifically binding to PD-1 comprises or consists of the amino acid sequence of SEQ ID NO: 1085.

In another embodiment, the ISVD specifically binding to human PD-1 may have a sequence identity of more than 90%, such as more than 95% or even more than 99%, with SEQ ID NO: 1086, wherein the CDRs are as defined above. In one embodiment, the ISVD specifically binding to PD-1 comprises or consists of the amino acid sequence of SEQ ID NO: 1086.

In another embodiment, the ISVD specifically binding to human PD-1 may have a sequence identity of more than 90%, such as more than 95% or even more than 99%, with SEQ ID NO: 1087, wherein the CDRs are as defined above. In one embodiment, the ISVD specifically binding to PD-1 comprises or consists of the amino acid sequence of SEQ ID NO: 1087.

In another embodiment, the ISVD specifically binding to human PD-1 may have a sequence identity of more than 90%, such as more than 95% or even more than 99%, with SEQ ID NO: 1088, wherein the CDRs are as defined above. In one embodiment, the ISVD specifically binding to PD-1 comprises or consists of the amino acid sequence of SEQ ID NO: 1088.

In another embodiment, the ISVD specifically binding to human PD-1 may have a sequence identity of more than 90%, such as more than 95% or even more than 99%, with SEQ ID NO: 1089, wherein the CDRs are as defined above. In one embodiment, the ISVD specifically binding to PD-1 comprises or consists of the amino acid sequence of SEQ ID NO: 1089.

In another embodiment, the ISVD specifically binding to human PD-1 may have a sequence identity of more than 90%, such as more than 95% or even more than 99%, with SEQ ID NO: 1090, wherein the CDRs are as defined above. In one embodiment, the ISVD specifically binding to PD-1 comprises or consists of the amino acid sequence of SEQ ID NO: 1090.

In another embodiment, the ISVD specifically binding to human PD-1 may have a sequence identity of more than 90%, such as more than 95% or even more than 99%, with SEQ ID NO: 1091, wherein the CDRs are as defined above. In one embodiment, the ISVD specifically binding to PD-1 comprises or consists of the amino acid sequence of SEQ ID NO: 1091.

In another embodiment, the ISVD specifically binding to human PD-1 may have a sequence identity of more than 90%, such as more than 95% or even more than 99%, with SEQ ID NO: 1092, wherein the CDRs are as defined above. In one embodiment, the ISVD specifically binding to PD-1 comprises or consists of the amino acid sequence of SEQ ID NO: 1092.

In another embodiment, the ISVD specifically binding to human PD-1 may have a sequence identity of more than 90%, such as more than 95% or even more than 99%, with SEQ ID NO: 1093, wherein the CDRs are as defined above. In one embodiment, the ISVD specifically binding to PD-1 comprises or consists of the amino acid sequence of SEQ ID NO: 1093.

In another embodiment, the ISVD specifically binding to human PD-1 may have a sequence identity of more than 90%, such as more than 95% or even more than 99%, with SEQ ID NO: 1094, wherein the CDRs are as defined above. In one embodiment, the ISVD specifically binding to PD-1 comprises or consists of the amino acid sequence of SEQ ID NO: 1094.

In another embodiment, the ISVD specifically binding to human PD-1 may have a sequence identity of more than 90%, such as more than 95% or even more than 99%, with SEQ ID NO: 1095, wherein the CDRs are as defined above. In one embodiment, the ISVD specifically binding to PD-1 comprises or consists of the amino acid sequence of SEQ ID NO: 1095.

In another embodiment, the ISVD specifically binding to human PD-1 may have a sequence identity of more than 90%, such as more than 95% or even more than 99%, with SEQ ID NO: 1096, wherein the CDRs are as defined above. In one embodiment, the ISVD specifically binding to PD-1 comprises or consists of the amino acid sequence of SEQ ID NO: 1096.

In another embodiment, the ISVD specifically binding to human PD-1 may have a sequence identity of more than 90%, such as more than 95% or even more than 99%, with SEQ ID NO: 1097, wherein the CDRs are as defined above. In one embodiment, the ISVD specifically binding to PD-1 comprises or consists of the amino acid sequence of SEQ ID NO: 1097.

In another embodiment, the ISVD specifically binding to human PD-1 may have a sequence identity of more than 90%, such as more than 95% or even more than 99%, with SEQ ID NO: 1098, wherein the CDRs are as defined above. In one embodiment, the ISVD specifically binding to PD-1 comprises or consists of the amino acid sequence of SEQ ID NO: 1098.

In another embodiment, the ISVD specifically binding to human PD-1 may have a sequence identity of more than 90%, such as more than 95% or even more than 99%, with SEQ ID NO: 1099, wherein the CDRs are as defined above. In one embodiment, the ISVD specifically binding to PD-1 comprises or consists of the amino acid sequence of SEQ ID NO: 1099.

In another embodiment, the ISVD specifically binding to human PD-1 may have a sequence identity of more than 90%, such as more than 95% or even more than 99%, with SEQ ID NO: 1100, wherein the CDRs are as defined above. In one embodiment, the ISVD specifically binding to PD-1 comprises or consists of the amino acid sequence of SEQ ID NO: 1100.

In another embodiment, the ISVD specifically binding to human PD-1 may have a sequence identity of more than 90%, such as more than 95% or even more than 99%, with SEQ ID NO: 1101, wherein the CDRs are as defined above. In one embodiment, the ISVD specifically binding to PD-1 comprises or consists of the amino acid sequence of SEQ ID NO: 1101.

In another embodiment, the ISVD specifically binding to human PD-1 may have a sequence identity of more than 90%, such as more than 95% or even more than 99%, with SEQ ID NO: 1102, wherein the CDRs are as defined above. In one embodiment, the ISVD specifically binding to PD-1 comprises or consists of the amino acid sequence of SEQ ID NO: 1102.

In another embodiment, the ISVD specifically binding to human PD-1 may have a sequence identity of more than 90%, such as more than 95% or even more than 99%, with SEQ ID NO: 1103, wherein the CDRs are as defined above. In one embodiment, the ISVD specifically binding to PD-1 comprises or consists of the amino acid sequence of SEQ ID NO: 1103.

In another embodiment, the ISVD specifically binding to human PD-1 may have a sequence identity of more than 90%, such as more than 95% or even more than 99%, with SEQ ID NO: 1104, wherein the CDRs are as defined above. In one embodiment, the ISVD specifically binding to PD-1 comprises or consists of the amino acid sequence of SEQ ID NO: 1104.

In another embodiment, the ISVD specifically binding to human PD-1 may have a sequence identity of more than 90%, such as more than 95% or even more than 99%, with SEQ ID NO: 1105, wherein the CDRs are as defined above. In one embodiment, the ISVD specifically binding to PD-1 comprises or consists of the amino acid sequence of SEQ ID NO: 1105.

In another embodiment, the ISVD specifically binding to human PD-1 may have a sequence identity of more than 90%, such as more than 95% or even more than 99%, with SEQ ID NO: 1106, wherein the CDRs are as defined above. In one embodiment, the ISVD specifically binding to PD-1 comprises or consists of the amino acid sequence of SEQ ID NO: 1106.

In another embodiment, the ISVD specifically binding to human PD-1 may have a sequence identity of more than 90%, such as more than 95% or even more than 99%, with SEQ ID NO: 1107, wherein the CDRs are as defined above. In one embodiment, the ISVD specifically binding to PD-1 comprises or consists of the amino acid sequence of SEQ ID NO: 1107.

In another embodiment, the ISVD specifically binding to human PD-1 may have a sequence identity of more than 90%, such as more than 95% or even more than 99%, with SEQ ID NO: 1108, wherein the CDRs are as defined above. In one embodiment, the ISVD specifically binding to PD-1 comprises or consists of the amino acid sequence of SEQ ID NO: 1108.

In another embodiment, the ISVD specifically binding to human PD-1 may have a sequence identity of more than 90%, such as more than 95% or even more than 99%, with SEQ ID NO: 1109, wherein the CDRs are as defined above. In one embodiment, the ISVD specifically binding to PD-1 comprises or consists of the amino acid sequence of SEQ ID NO: 1109.

In another embodiment, the ISVD specifically binding to human PD-1 may have a sequence identity of more than 90%, such as more than 95% or even more than 99%, with SEQ ID NO: 1110, wherein the CDRs are as defined above. In one embodiment, the ISVD specifically binding to PD-1 comprises or consists of the amino acid sequence of SEQ ID NO: 1110.

In another embodiment, the ISVD specifically binding to human PD-1 may have a sequence identity of more than 90%, such as more than 95% or even more than 99%, with SEQ ID NO: 1111, wherein the CDRs are as defined above. In one embodiment, the ISVD specifically binding to PD-1 comprises or consists of the amino acid sequence of SEQ ID NO: 1111.

In another embodiment, the ISVD specifically binding to human PD-1 may have a sequence identity of more than 90%, such as more than 95% or even more than 99%, with SEQ ID NO: 1112, wherein the CDRs are as defined above. In one embodiment, the ISVD specifically binding to PD-1 comprises or consists of the amino acid sequence of SEQ ID NO: 1112.

In another embodiment, the ISVD specifically binding to human PD-1 may have a sequence identity of more than 90%, such as more than 95% or even more than 99%, with SEQ ID NO: 1113, wherein the CDRs are as defined above. In one embodiment, the ISVD specifically binding to PD-1 comprises or consists of the amino acid sequence of SEQ ID NO: 1113.

In another embodiment, the ISVD specifically binding to human PD-1 may have a sequence identity of more than 90%, such as more than 95% or even more than 99%, with SEQ ID NO: 1114, wherein the CDRs are as defined above. In one embodiment, the ISVD specifically binding to PD-1 comprises or consists of the amino acid sequence of SEQ ID NO: 1114.

In another embodiment, the ISVD specifically binding to human PD-1 may have a sequence identity of more than 90%, such as more than 95% or even more than 99%, with SEQ ID NO: 1115, wherein the CDRs are as defined above. In one embodiment, the ISVD specifically binding to PD-1 comprises or consists of the amino acid sequence of SEQ ID NO: 1115.

In another embodiment, the ISVD specifically binding to human PD-1 may have a sequence identity of more than 90%, such as more than 95% or even more than 99%, with SEQ ID NO: 1116, wherein the CDRs are as defined above. In one embodiment, the ISVD specifically binding to PD-1 comprises or consists of the amino acid sequence of SEQ ID NO: 1116.

In another embodiment, the ISVD specifically binding to human PD-1 may have a sequence identity of more than 90%, such as more than 95% or even more than 99%, with SEQ ID NO: 1117, wherein the CDRs are as defined above. In one embodiment, the ISVD specifically binding to PD-1 comprises or consists of the amino acid sequence of SEQ ID NO: 1117.

In another embodiment, the ISVD specifically binding to human PD-1 may have a sequence identity of more than 90%, such as more than 95% or even more than 99%, with SEQ ID NO: 1118, wherein the CDRs are as defined above. In one embodiment, the ISVD specifically binding to PD-1 comprises or consists of the amino acid sequence of SEQ ID NO: 1118.

In another embodiment, the ISVD specifically binding to human PD-1 may have a sequence identity of more than 90%, such as more than 95% or even more than 99%, with SEQ ID NO: 1119, wherein the CDRs are as defined above. In one embodiment, the ISVD specifically binding to PD-1 comprises or consists of the amino acid sequence of SEQ ID NO: 1119.

In another embodiment, the ISVD specifically binding to human PD-1 may have a sequence identity of more than 90%, such as more than 95% or even more than 99%, with SEQ ID NO: 1120, wherein the CDRs are as defined above. In one embodiment, the ISVD specifically binding to PD-1 comprises or consists of the amino acid sequence of SEQ ID NO: 1120.

In another embodiment, the ISVD specifically binding to human PD-1 may have a sequence identity of more than 90%, such as more than 95% or even more than 99%, with SEQ ID NO: 1121, wherein the CDRs are as defined above. In one embodiment, the ISVD specifically binding to PD-1 comprises or consists of the amino acid sequence of SEQ ID NO: 1121.

In another embodiment, the ISVD specifically binding to human PD-1 may have a sequence identity of more than 90%, such as more than 95% or even more than 99%, with SEQ ID NO: 1122, wherein the CDRs are as defined above. In one embodiment, the ISVD specifically binding to PD-1 comprises or consists of the amino acid sequence of SEQ ID NO: 1122.

In another embodiment, the ISVD specifically binding to human PD-1 may have a sequence identity of more than 90%, such as more than 95% or even more than 99%, with SEQ ID NO: 1123, wherein the CDRs are as defined above. In one embodiment, the ISVD specifically binding to PD-1 comprises or consists of the amino acid sequence of SEQ ID NO: 1123.

In another embodiment, the ISVD specifically binding to human PD-1 may have a sequence identity of more than 90%, such as more than 95% or even more than 99%, with SEQ ID NO: 1124, wherein the CDRs are as defined above. In one embodiment, the ISVD specifically binding to PD-1 comprises or consists of the amino acid sequence of SEQ ID NO: 1124.

In another embodiment, the ISVD specifically binding to human PD-1 may have a sequence identity of more than 90%, such as more than 95% or even more than 99%, with SEQ ID NO: 1125, wherein the CDRs are as defined above. In one embodiment, the ISVD specifically binding to PD-1 comprises or consists of the amino acid sequence of SEQ ID NO: 1125.

In another embodiment, the ISVD specifically binding to human PD-1 may have a sequence identity of more than 90%, such as more than 95% or even more than 99%, with SEQ ID NO: 1126, wherein the CDRs are as defined above. In one embodiment, the ISVD specifically binding to PD-1 comprises or consists of the amino acid sequence of SEQ ID NO: 1126.

In another embodiment, the ISVD specifically binding to human PD-1 may have a sequence identity of more than 90%, such as more than 95% or even more than 99%, with SEQ ID NO: 1127, wherein the CDRs are as defined above. In one embodiment, the ISVD specifically binding to PD-1 comprises or consists of the amino acid sequence of SEQ ID NO: 1127.

In another embodiment, the ISVD specifically binding to human PD-1 may have a sequence identity of more than 90%, such as more than 95% or even more than 99%, with SEQ ID NO: 1128, wherein the CDRs are as defined above. In one embodiment, the ISVD specifically binding to PD-1 comprises or consists of the amino acid sequence of SEQ ID NO: 1128.

In another embodiment, the ISVD specifically binding to human PD-1 may have a sequence identity of more than 90%, such as more than 95% or even more than 99%, with SEQ ID NO: 1129, wherein the CDRs are as defined above. In one embodiment, the ISVD specifically binding to PD-1 comprises or consists of the amino acid sequence of SEQ ID NO: 1129.

In another embodiment, the ISVD specifically binding to human PD-1 may have a sequence identity of more than 90%, such as more than 95% or even more than 99%, with SEQ ID NO: 1130, wherein the CDRs are as defined above. In one embodiment, the ISVD specifically binding to PD-1 comprises or consists of the amino acid sequence of SEQ ID NO: 1130.

In another embodiment, the ISVD specifically binding to human PD-1 may have a sequence identity of more than 90%, such as more than 95% or even more than 99%, with SEQ ID NO: 1131, wherein the CDRs are as defined above. In one embodiment, the ISVD specifically binding to PD-1 comprises or consists of the amino acid sequence of SEQ ID NO: 1131.

In another embodiment, the ISVD specifically binding to human PD-1 may have a sequence identity of more than 90%, such as more than 95% or even more than 99%, with SEQ ID NO: 1132, wherein the CDRs are as defined above. In one embodiment, the ISVD specifically binding to PD-1 comprises or consists of the amino acid sequence of SEQ ID NO: 1132.

In another embodiment, the ISVD specifically binding to human PD-1 may have a sequence identity of more than 90%, such as more than 95% or even more than 99%, with SEQ ID NO: 1133, wherein the CDRs are as defined above. In one embodiment, the ISVD specifically binding to PD-1 comprises or consists of the amino acid sequence of SEQ ID NO: 1133.

In another embodiment, the ISVD specifically binding to human PD-1 may have a sequence identity of more than 90%, such as more than 95% or even more than 99%, with SEQ ID NO: 1134, wherein the CDRs are as defined above. In one embodiment, the ISVD specifically binding to PD-1 comprises or consists of the amino acid sequence of SEQ ID NO: 1134.

In another embodiment, the ISVD specifically binding to human PD-1 may have a sequence identity of more than 90%, such as more than 95% or even more than 99%, with SEQ ID NO: 1135, wherein the CDRs are as defined above. In one embodiment, the ISVD specifically binding to PD-1 comprises or consists of the amino acid sequence of SEQ ID NO: 1135.

In another embodiment, the ISVD specifically binding to human PD-1 may have a sequence identity of more than 90%, such as more than 95% or even more than 99%, with SEQ ID NO: 1136, wherein the CDRs are as defined above. In one embodiment, the ISVD specifically binding to PD-1 comprises or consists of the amino acid sequence of SEQ ID NO: 1136.

In another embodiment, the ISVD specifically binding to human PD-1 may have a sequence identity of more than 90%, such as more than 95% or even more than 99%, with SEQ ID NO: 1137, wherein the CDRs are as defined above. In one embodiment, the ISVD specifically binding to PD-1 comprises or consists of the amino acid sequence of SEQ ID NO: 1137.

In another embodiment, the ISVD specifically binding to human PD-1 may have a sequence identity of more than 90%, such as more than 95% or even more than 99%, with SEQ ID NO: 1138, wherein the CDRs are as defined above. In one embodiment, the ISVD specifically binding to PD-1 comprises or consists of the amino acid sequence of SEQ ID NO: 1138.

In another embodiment, the ISVD specifically binding to human PD-1 may have a sequence identity of more than 90%, such as more than 95% or even more than 99%, with SEQ ID NO: 1139, wherein the CDRs are as defined above. In one embodiment, the ISVD specifically binding to PD-1 comprises or consists of the amino acid sequence of SEQ ID NO: 1139.

In another embodiment, the ISVD specifically binding to human PD-1 may have a sequence identity of more than 90%, such as more than 95% or even more than 99%, with SEQ ID NO: 1140, wherein the CDRs are as defined above. In one embodiment, the ISVD specifically binding to PD-1 comprises or consists of the amino acid sequence of SEQ ID NO: 1140.

In another embodiment, the ISVD specifically binding to human PD-1 may have a sequence identity of more than 90%, such as more than 95% or even more than 99%, with SEQ ID NO: 1141, wherein the CDRs are as defined above. In one embodiment, the ISVD specifically binding to PD-1 comprises or consists of the amino acid sequence of SEQ ID NO: 1141.

In another embodiment, the ISVD specifically binding to human PD-1 may have a sequence identity of more than 90%, such as more than 95% or even more than 99%, with SEQ ID NO: 1142, wherein the CDRs are as defined above. In one embodiment, the ISVD specifically binding to PD-1 comprises or consists of the amino acid sequence of SEQ ID NO: 1142.

In another embodiment, the ISVD specifically binding to human PD-1 may have a sequence identity of more than 90%, such as more than 95% or even more than 99%, with SEQ ID NO: 1143, wherein the CDRs are as defined above. In one embodiment, the ISVD specifically binding to PD-1 comprises or consists of the amino acid sequence of SEQ ID NO: 1143.

In another embodiment, the ISVD specifically binding to human PD-1 may have a sequence identity of more than 90%, such as more than 95% or even more than 99%, with SEQ ID NO: 1144, wherein the CDRs are as defined above. In one embodiment, the ISVD specifically binding to PD-1 comprises or consists of the amino acid sequence of SEQ ID NO: 1144.

In another embodiment, the ISVD specifically binding to human PD-1 may have a sequence identity of more than 90%, such as more than 95% or even more than 99%, with SEQ ID NO: 1145, wherein the CDRs are as defined above. In one embodiment, the ISVD specifically binding to PD-1 comprises or consists of the amino acid sequence of SEQ ID NO: 1145.

In another embodiment, the ISVD specifically binding to human PD-1 may have a sequence identity of more than 90%, such as more than 95% or even more than 99%, with SEQ ID NO: 1146, wherein the CDRs are as defined above. In one embodiment, the ISVD specifically binding to PD-1 comprises or consists of the amino acid sequence of SEQ ID NO: 1146.

In another embodiment, the ISVD specifically binding to human PD-1 may have a sequence identity of more than 90%, such as more than 95% or even more than 99%, with SEQ ID NO: 1147, wherein the CDRs are as defined above. In one embodiment, the ISVD specifically binding to PD-1 comprises or consists of the amino acid sequence of SEQ ID NO: 1147.

In another embodiment, the ISVD specifically binding to human PD-1 may have a sequence identity of more than 90%, such as more than 95% or even more than 99%, with SEQ ID NO: 1148, wherein the CDRs are as defined above. In one embodiment, the ISVD specifically binding to PD-1 comprises or consists of the amino acid sequence of SEQ ID NO: 1148.

In another embodiment, the ISVD specifically binding to human PD-1 may have a sequence identity of more than 90%, such as more than 95% or even more than 99%, with SEQ ID NO: 1149, wherein the CDRs are as defined above. In one embodiment, the ISVD specifically binding to PD-1 comprises or consists of the amino acid sequence of SEQ ID NO: 1149.

In another embodiment, the ISVD specifically binding to human PD-1 may have a sequence identity of more than 90%, such as more than 95% or even more than 99%, with SEQ ID NO: 1150, wherein the CDRs are as defined above. In one embodiment, the ISVD specifically binding to PD-1 comprises or consists of the amino acid sequence of SEQ ID NO: 1150.

In another embodiment, the ISVD specifically binding to human PD-1 may have a sequence identity of more than 90%, such as more than 95% or even more than 99%, with SEQ ID NO: 1151, wherein the CDRs are as defined above. In one embodiment, the ISVD specifically binding to PD-1 comprises or consists of the amino acid sequence of SEQ ID NO: 1151.

In another embodiment, the ISVD specifically binding to human PD-1 may have a sequence identity of more than 90%, such as more than 95% or even more than 99%, with SEQ ID NO: 1152, wherein the CDRs are as defined above. In one embodiment, the ISVD specifically binding to PD-1 comprises or consists of the amino acid sequence of SEQ ID NO: 1152.

In another embodiment, the ISVD specifically binding to human PD-1 may have a sequence identity of more than 90%, such as more than 95% or even more than 99%, with SEQ ID NO: 1153, wherein the CDRs are as defined above. In one embodiment, the ISVD specifically binding to PD-1 comprises or consists of the amino acid sequence of SEQ ID NO: 1153.

In another embodiment, the ISVD specifically binding to human PD-1 may have a sequence identity of more than 90%, such as more than 95% or even more than 99%, with SEQ ID NO: 1154, wherein the CDRs are as defined above. In one embodiment, the ISVD specifically binding to PD-1 comprises or consists of the amino acid sequence of SEQ ID NO: 1154.

In another embodiment, the ISVD specifically binding to human PD-1 may have a sequence identity of more than 90%, such as more than 95% or even more than 99%, with SEQ ID NO: 1155, wherein the CDRs are as defined above. In one embodiment, the ISVD specifically binding to PD-1 comprises or consists of the amino acid sequence of SEQ ID NO: 1155.

In another embodiment, the ISVD specifically binding to human PD-1 may have a sequence identity of more than 90%, such as more than 95% or even more than 99%, with SEQ ID NO: 1156, wherein the CDRs are as defined above. In one embodiment, the ISVD specifically binding to PD-1 comprises or consists of the amino acid sequence of SEQ ID NO: 1156.

In another embodiment, the ISVD specifically binding to human PD-1 may have a sequence identity of more than 90%, such as more than 95% or even more than 99%, with SEQ ID NO: 1157, wherein the CDRs are as defined above. In one embodiment, the ISVD specifically binding to PD-1 comprises or consists of the amino acid sequence of SEQ ID NO: 1157.

In another embodiment, the ISVD specifically binding to human PD-1 may have a sequence identity of more than 90%, such as more than 95% or even more than 99%, with SEQ ID NO: 1158, wherein the CDRs are as defined above. In one embodiment, the ISVD specifically binding to PD-1 comprises or consists of the amino acid sequence of SEQ ID NO: 1158.

In another embodiment, the ISVD specifically binding to human PD-1 may have a sequence identity of more than 90%, such as more than 95% or even more than 99%, with SEQ ID NO: 1159, wherein the CDRs are as defined above. In one embodiment, the ISVD specifically binding to PD-1 comprises or consists of the amino acid sequence of SEQ ID NO: 1159.

In another embodiment, the ISVD specifically binding to human PD-1 may have a sequence identity of more than 90%, such as more than 95% or even more than 99%, with SEQ ID NO: 1160, wherein the CDRs are as defined above. In one embodiment, the ISVD specifically binding to PD-1 comprises or consists of the amino acid sequence of SEQ ID NO: 1160.

In another embodiment, the ISVD specifically binding to human PD-1 may have a sequence identity of more than 90%, such as more than 95% or even more than 99%, with SEQ ID NO: 1161, wherein the CDRs are as defined above. In one embodiment, the ISVD specifically binding to PD-1 comprises or consists of the amino acid sequence of SEQ ID NO: 1161.

In another embodiment, the ISVD specifically binding to human PD-1 may have a sequence identity of more than 90%, such as more than 95% or even more than 99%, with SEQ ID NO: 1162, wherein the CDRs are as defined above. In one embodiment, the ISVD specifically binding to PD-1 comprises or consists of the amino acid sequence of SEQ ID NO: 1162.

In another embodiment, the ISVD specifically binding to human PD-1 may have a sequence identity of more than 90%, such as more than 95% or even more than 99%, with SEQ ID NO: 1163, wherein the CDRs are as defined above. In one embodiment, the ISVD specifically binding to PD-1 comprises or consists of the amino acid sequence of SEQ ID NO: 1163.

In another embodiment, the ISVD specifically binding to human PD-1 may have a sequence identity of more than 90%, such as more than 95% or even more than 99%, with SEQ ID NO: 1164, wherein the CDRs are as defined above. In one embodiment, the ISVD specifically binding to PD-1 comprises or consists of the amino acid sequence of SEQ ID NO: 1164.

In another embodiment, the ISVD specifically binding to human PD-1 may have a sequence identity of more than 90%, such as more than 95% or even more than 99%, with SEQ ID NO: 1165, wherein the CDRs are as defined above. In one embodiment, the ISVD specifically binding to PD-1 comprises or consists of the amino acid sequence of SEQ ID NO: 1165.

In another embodiment, the ISVD specifically binding to human PD-1 may have a sequence identity of more than 90%, such as more than 95% or even more than 99%, with SEQ ID NO: 1166, wherein the CDRs are as defined above. In one embodiment, the ISVD specifically binding to PD-1 comprises or consists of the amino acid sequence of SEQ ID NO: 1166.

In another embodiment, the ISVD specifically binding to human PD-1 may have a sequence identity of more than 90%, such as more than 95% or even more than 99%, with SEQ ID NO: 1167, wherein the CDRs are as defined above. In one embodiment, the ISVD specifically binding to PD-1 comprises or consists of the amino acid sequence of SEQ ID NO: 1167.

In another embodiment, the ISVD specifically binding to human PD-1 may have a sequence identity of more than 90%, such as more than 95% or even more than 99%, with SEQ ID NO: 1168, wherein the CDRs are as defined above. In one embodiment, the ISVD specifically binding to PD-1 comprises or consists of the amino acid sequence of SEQ ID NO: 1168.

In another embodiment, the ISVD specifically binding to human PD-1 may have a sequence identity of more than 90%, such as more than 95% or even more than 99%, with SEQ ID NO: 1169, wherein the CDRs are as defined above. In one embodiment, the ISVD specifically binding to PD-1 comprises or consists of the amino acid sequence of SEQ ID NO: 1169.

In another embodiment, the ISVD specifically binding to human PD-1 may have a sequence identity of more than 90%, such as more than 95% or even more than 99%, with SEQ ID NO: 1170, wherein the CDRs are as defined above. In one embodiment, the ISVD specifically binding to PD-1 comprises or consists of the amino acid sequence of SEQ ID NO: 1170.

In another embodiment, the ISVD specifically binding to human PD-1 may have a sequence identity of more than 90%, such as more than 95% or even more than 99%, with SEQ ID NO: 1171, wherein the CDRs are as defined above. In one embodiment, the ISVD specifically binding to PD-1 comprises or consists of the amino acid sequence of SEQ ID NO: 1171.

In another embodiment, the ISVD specifically binding to human PD-1 may have a sequence identity of more than 90%, such as more than 95% or even more than 99%, with SEQ ID NO: 1172, wherein the CDRs are as defined above. In one embodiment, the ISVD specifically binding to PD-1 comprises or consists of the amino acid sequence of SEQ ID NO: 1172.

In another embodiment, the ISVD specifically binding to human PD-1 may have a sequence identity of more than 90%, such as more than 95% or even more than 99%, with SEQ ID NO: 1173, wherein the CDRs are as defined above. In one embodiment, the ISVD specifically binding to PD-1 comprises or consists of the amino acid sequence of SEQ ID NO: 1173.

In another embodiment, the ISVD specifically binding to human PD-1 may have a sequence identity of more than 90%, such as more than 95% or even more than 99%, with SEQ ID NO: 1174, wherein the CDRs are as defined above. In one embodiment, the ISVD specifically binding to PD-1 comprises or consists of the amino acid sequence of SEQ ID NO: 1174.

In another embodiment, the ISVD specifically binding to human PD-1 may have a sequence identity of more than 90%, such as more than 95% or even more than 99%, with SEQ ID NO: 1175, wherein the CDRs are as defined above. In one embodiment, the ISVD specifically binding to PD-1 comprises or consists of the amino acid sequence of SEQ ID NO: 1175.

In another embodiment, the ISVD specifically binding to human PD-1 may have a sequence identity of more than 90%, such as more than 95% or even more than 99%, with SEQ ID NO: 1176, wherein the CDRs are as defined above. In one embodiment, the ISVD specifically binding to PD-1 comprises or consists of the amino acid sequence of SEQ ID NO: 1176.

In another embodiment, the ISVD specifically binding to human PD-1 may have a sequence identity of more than 90%, such as more than 95% or even more than 99%, with SEQ ID NO: 1177, wherein the CDRs are as defined above. In one embodiment, the ISVD specifically binding to PD-1 comprises or consists of the amino acid sequence of SEQ ID NO: 1177.

In another embodiment, the ISVD specifically binding to human PD-1 may have a sequence identity of more than 90%, such as more than 95% or even more than 99%, with SEQ ID NO: 1178, wherein the CDRs are as defined above. In one embodiment, the ISVD specifically binding to PD-1 comprises or consists of the amino acid sequence of SEQ ID NO: 1178.

In another embodiment, the ISVD specifically binding to human PD-1 may have a sequence identity of more than 90%, such as more than 95% or even more than 99%, with SEQ ID NO: 1179, wherein the CDRs are as defined above. In one embodiment, the ISVD specifically binding to PD-1 comprises or consists of the amino acid sequence of SEQ ID NO: 1179.

In another embodiment, the ISVD specifically binding to human PD-1 may have a sequence identity of more than 90%, such as more than 95% or even more than 99%, with SEQ ID NO: 1180, wherein the CDRs are as defined above. In one embodiment, the ISVD specifically binding to PD-1 comprises or consists of the amino acid sequence of SEQ ID NO: 1180.

In another embodiment, the ISVD specifically binding to human PD-1 may have a sequence identity of more than 90%, such as more than 95% or even more than 99%, with SEQ ID NO: 1181, wherein the CDRs are as defined above. In one embodiment, the ISVD specifically binding to PD-1 comprises or consists of the amino acid sequence of SEQ ID NO: 1181.

In another embodiment, the ISVD specifically binding to human PD-1 may have a sequence identity of more than 90%, such as more than 95% or even more than 99%, with SEQ ID NO: 1182, wherein the CDRs are as defined above. In one embodiment, the ISVD specifically binding to PD-1 comprises or consists of the amino acid sequence of SEQ ID NO: 1182.

In another embodiment, the ISVD specifically binding to human PD-1 may have a sequence identity of more than 90%, such as more than 95% or even more than 99%, with SEQ ID NO: 1183, wherein the CDRs are as defined above. In one embodiment, the ISVD specifically binding to PD-1 comprises or consists of the amino acid sequence of SEQ ID NO: 1183.

In another embodiment, the ISVD specifically binding to human PD-1 may have a sequence identity of more than 90%, such as more than 95% or even more than 99%, with SEQ ID NO: 1184, wherein the CDRs are as defined above. In one embodiment, the ISVD specifically binding to PD-1 comprises or consists of the amino acid sequence of SEQ ID NO: 1184.

In another embodiment, the ISVD specifically binding to human PD-1 may have a sequence identity of more than 90%, such as more than 95% or even more than 99%, with SEQ ID NO: 1185, wherein the CDRs are as defined above. In one embodiment, the ISVD specifically binding to PD-1 comprises or consists of the amino acid sequence of SEQ ID NO: 1185.

In another embodiment, the ISVD specifically binding to human PD-1 may have a sequence identity of more than 90%, such as more than 95% or even more than 99%, with SEQ ID NO: 1186, wherein the CDRs are as defined above. In one embodiment, the ISVD specifically binding to PD-1 comprises or consists of the amino acid sequence of SEQ ID NO: 1186.

In another embodiment, the ISVD specifically binding to human PD-1 may have a sequence identity of more than 90%, such as more than 95% or even more than 99%, with SEQ ID NO: 1187, wherein the CDRs are as defined above. In one embodiment, the ISVD specifically binding to PD-1 comprises or consists of the amino acid sequence of SEQ ID NO: 1187.

In another embodiment, the ISVD specifically binding to human PD-1 may have a sequence identity of more than 90%, such as more than 95% or even more than 99%, with SEQ ID NO: 1188, wherein the CDRs are as defined above. In one embodiment, the ISVD specifically binding to PD-1 comprises or consists of the amino acid sequence of SEQ ID NO: 1188.

In another embodiment, the ISVD specifically binding to human PD-1 may have a sequence identity of more than 90%, such as more than 95% or even more than 99%, with SEQ ID NO: 1189, wherein the CDRs are as defined above. In one embodiment, the ISVD specifically binding to PD-1 comprises or consists of the amino acid sequence of SEQ ID NO: 1189.

In another embodiment, the ISVD specifically binding to human PD-1 may have a sequence identity of more than 90%, such as more than 95% or even more than 99%, with SEQ ID NO: 1190, wherein the CDRs are as defined above. In one embodiment, the ISVD specifically binding to PD-1 comprises or consists of the amino acid sequence of SEQ ID NO: 1190.

In another embodiment, the ISVD specifically binding to human PD-1 may have a sequence identity of more than 90%, such as more than 95% or even more than 99%, with SEQ ID NO: 1191, wherein the CDRs are as defined above. In one embodiment, the ISVD specifically binding to PD-1 comprises or consists of the amino acid sequence of SEQ ID NO: 1191.

In another embodiment, the ISVD specifically binding to human PD-1 may have a sequence identity of more than 90%, such as more than 95% or even more than 99%, with SEQ ID NO: 1192, wherein the CDRs are as defined above. In one embodiment, the ISVD specifically binding to PD-1 comprises or consists of the amino acid sequence of SEQ ID NO: 1192.

In another embodiment, the ISVD specifically binding to human PD-1 may have a sequence identity of more than 90%, such as more than 95% or even more than 99%, with SEQ ID NO: 1193, wherein the CDRs are as defined above. In one embodiment, the ISVD specifically binding to PD-1 comprises or consists of the amino acid sequence of SEQ ID NO: 1193.

In another embodiment, the ISVD specifically binding to human PD-1 may have a sequence identity of more than 90%, such as more than 95% or even more than 99%, with SEQ ID NO: 1194, wherein the CDRs are as defined above. In one embodiment, the ISVD specifically binding to PD-1 comprises or consists of the amino acid sequence of SEQ ID NO: 1194.

In another embodiment, the ISVD specifically binding to human PD-1 may have a sequence identity of more than 90%, such as more than 95% or even more than 99%, with SEQ ID NO: 1195, wherein the CDRs are as defined above. In one embodiment, the ISVD specifically binding to PD-1 comprises or consists of the amino acid sequence of SEQ ID NO: 1195.

In another embodiment, the ISVD specifically binding to human PD-1 may have a sequence identity of more than 90%, such as more than 95% or even more than 99%, with SEQ ID NO: 1196, wherein the CDRs are as defined above. In one embodiment, the ISVD specifically binding to PD-1 comprises or consists of the amino acid sequence of SEQ ID NO: 1196.

In another embodiment, the ISVD specifically binding to human PD-1 may have a sequence identity of more than 90%, such as more than 95% or even more than 99%, with SEQ ID NO: 1197, wherein the CDRs are as defined above. In one embodiment, the ISVD specifically binding to PD-1 comprises or consists of the amino acid sequence of SEQ ID NO: 1197.

In another embodiment, the ISVD specifically binding to human PD-1 may have a sequence identity of more than 90%, such as more than 95% or even more than 99%, with SEQ ID NO: 1198, wherein the CDRs are as defined above. In one embodiment, the ISVD specifically binding to PD-1 comprises or consists of the amino acid sequence of SEQ ID NO: 1198.

In another embodiment, the ISVD specifically binding to human PD-1 may have a sequence identity of more than 90%, such as more than 95% or even more than 99%, with SEQ ID NO: 1199, wherein the CDRs are as defined above. In one embodiment, the ISVD specifically binding to PD-1 comprises or consists of the amino acid sequence of SEQ ID NO: 1199.

In another embodiment, the ISVD specifically binding to human PD-1 may have a sequence identity of more than 90%, such as more than 95% or even more than 99%, with SEQ ID NO: 1200, wherein the CDRs are as defined above. In one embodiment, the ISVD specifically binding to PD-1 comprises or consists of the amino acid sequence of SEQ ID NO: 1200.

In another embodiment, the ISVD specifically binding to human PD-1 may have a sequence identity of more than 90%, such as more than 95% or even more than 99%, with SEQ ID NO: 1201, wherein the CDRs are as defined above. In one embodiment, the ISVD specifically binding to PD-1 comprises or consists of the amino acid sequence of SEQ ID NO: 1201.

In another embodiment, the ISVD specifically binding to human PD-1 may have a sequence identity of more than 90%, such as more than 95% or even more than 99%, with SEQ ID NO: 1202, wherein the CDRs are as defined above. In one embodiment, the ISVD specifically binding to PD-1 comprises or consists of the amino acid sequence of SEQ ID NO: 1202.

In another embodiment, the ISVD specifically binding to human PD-1 may have a sequence identity of more than 90%, such as more than 95% or even more than 99%, with SEQ ID NO: 1203, wherein the CDRs are as defined above. In one embodiment, the ISVD specifically binding to PD-1 comprises or consists of the amino acid sequence of SEQ ID NO: 1203.

In another embodiment, the ISVD specifically binding to human PD-1 may have a sequence identity of more than 90%, such as more than 95% or even more than 99%, with SEQ ID NO: 1204, wherein the CDRs are as defined above. In one embodiment, the ISVD specifically binding to PD-1 comprises or consists of the amino acid sequence of SEQ ID NO: 1204.

In another embodiment, the ISVD specifically binding to human PD-1 may have a sequence identity of more than 90%, such as more than 95% or even more than 99%, with SEQ ID NO: 1205, wherein the CDRs are as defined above. In one embodiment, the ISVD specifically binding to PD-1 comprises or consists of the amino acid sequence of SEQ ID NO: 1205.

In another embodiment, the ISVD specifically binding to human PD-1 may have a sequence identity of more than 90%, such as more than 95% or even more than 99%, with SEQ ID NO: 1206, wherein the CDRs are as defined above. In one embodiment, the ISVD specifically binding to PD-1 comprises or consists of the amino acid sequence of SEQ ID NO: 1206.

In another embodiment, the ISVD specifically binding to human PD-1 may have a sequence identity of more than 90%, such as more than 95% or even more than 99%, with SEQ ID NO: 1207, wherein the CDRs are as defined above. In one embodiment, the ISVD specifically binding to PD-1 comprises or consists of the amino acid sequence of SEQ ID NO: 1207.

In another embodiment, the ISVD specifically binding to human PD-1 may have a sequence identity of more than 90%, such as more than 95% or even more than 99%, with SEQ ID NO: 1208, wherein the CDRs are as defined above. In one embodiment, the ISVD specifically binding to PD-1 comprises or consists of the amino acid sequence of SEQ ID NO: 1208.

In another embodiment, the ISVD specifically binding to human PD-1 may have a sequence identity of more than 90%, such as more than 95% or even more than 99%, with SEQ ID NO: 1209, wherein the CDRs are as defined above. In one embodiment, the ISVD specifically binding to PD-1 comprises or consists of the amino acid sequence of SEQ ID NO: 1209.

In another embodiment, the ISVD specifically binding to human PD-1 may have a sequence identity of more than 90%, such as more than 95% or even more than 99%, with SEQ ID NO: 1210, wherein the CDRs are as defined above. In one embodiment, the ISVD specifically binding to PD-1 comprises or consists of the amino acid sequence of SEQ ID NO: 1210.

In another embodiment, the ISVD specifically binding to human PD-1 may have a sequence identity of more than 90%, such as more than 95% or even more than 99%, with SEQ ID NO: 1211, wherein the CDRs are as defined above. In one embodiment, the ISVD specifically binding to PD-1 comprises or consists of the amino acid sequence of SEQ ID NO: 1211.

In another embodiment, the ISVD specifically binding to human PD-1 may have a sequence identity of more than 90%, such as more than 95% or even more than 99%, with SEQ ID NO: 1212, wherein the CDRs are as defined above. In one embodiment, the ISVD specifically binding to PD-1 comprises or consists of the amino acid sequence of SEQ ID NO: 1212.

In another embodiment, the ISVD specifically binding to human PD-1 may have a sequence identity of more than 90%, such as more than 95% or even more than 99%, with SEQ ID NO: 1213, wherein the CDRs are as defined above. In one embodiment, the ISVD specifically binding to PD-1 comprises or consists of the amino acid sequence of SEQ ID NO: 1213.

In another embodiment, the ISVD specifically binding to human PD-1 may have a sequence identity of more than 90%, such as more than 95% or even more than 99%, with SEQ ID NO: 1214, wherein the CDRs are as defined above. In one embodiment, the ISVD specifically binding to PD-1 comprises or consists of the amino acid sequence of SEQ ID NO: 1214.

In another embodiment, the ISVD specifically binding to human PD-1 may have a sequence identity of more than 90%, such as more than 95% or even more than 99%, with SEQ ID NO: 1215, wherein the CDRs are as defined above. In one embodiment, the ISVD specifically binding to PD-1 comprises or consists of the amino acid sequence of SEQ ID NO: 1215.

In another embodiment, the ISVD specifically binding to human PD-1 may have a sequence identity of more than 90%, such as more than 95% or even more than 99%, with SEQ ID NO: 1216, wherein the CDRs are as defined above. In one embodiment, the ISVD specifically binding to PD-1 comprises or consists of the amino acid sequence of SEQ ID NO: 1216.

In another embodiment, the ISVD specifically binding to human PD-1 may have a sequence identity of more than 90%, such as more than 95% or even more than 99%, with SEQ ID NO: 1217, wherein the CDRs are as defined above. In one embodiment, the ISVD specifically binding to PD-1 comprises or consists of the amino acid sequence of SEQ ID NO: 1217.

In another embodiment, the ISVD specifically binding to human PD-1 may have a sequence identity of more than 90%, such as more than 95% or even more than 99%, with SEQ ID NO: 1218, wherein the CDRs are as defined above. In one embodiment, the ISVD specifically binding to PD-1 comprises or consists of the amino acid sequence of SEQ ID NO: 1218.

In another embodiment, the ISVD specifically binding to human PD-1 may have a sequence identity of more than 90%, such as more than 95% or even more than 99%, with SEQ ID NO: 1219, wherein the CDRs are as defined above. In one embodiment, the ISVD specifically binding to PD-1 comprises or consists of the amino acid sequence of SEQ ID NO: 1219.

In another embodiment, the ISVD specifically binding to human PD-1 may have a sequence identity of more than 90%, such as more than 95% or even more than 99%, with SEQ ID NO: 1220, wherein the CDRs are as defined above. In one embodiment, the ISVD specifically binding to PD-1 comprises or consists of the amino acid sequence of SEQ ID NO: 1220.

In another embodiment, the ISVD specifically binding to human PD-1 may have a sequence identity of more than 90%, such as more than 95% or even more than 99%, with SEQ ID NO: 1221, wherein the CDRs are as defined above. In one embodiment, the ISVD specifically binding to PD-1 comprises or consists of the amino acid sequence of SEQ ID NO: 1221.

In another embodiment, the ISVD specifically binding to human PD-1 may have a sequence identity of more than 90%, such as more than 95% or even more than 99%, with SEQ ID NO: 1222, wherein the CDRs are as defined above. In one embodiment, the ISVD specifically binding to PD-1 comprises or consists of the amino acid sequence of SEQ ID NO: 1222.

In another embodiment, the ISVD specifically binding to human PD-1 may have a sequence identity of more than 90%, such as more than 95% or even more than 99%, with SEQ ID NO: 1223, wherein the CDRs are as defined above. In one embodiment, the ISVD specifically binding to PD-1 comprises or consists of the amino acid sequence of SEQ ID NO: 1223.

In another embodiment, the ISVD specifically binding to human PD-1 may have a sequence identity of more than 90%, such as more than 95% or even more than 99%, with SEQ ID NO: 1224, wherein the CDRs are as defined above. In one embodiment, the ISVD specifically binding to PD-1 comprises or consists of the amino acid sequence of SEQ ID NO: 1224.

In another embodiment, the ISVD specifically binding to human PD-1 may have a sequence identity of more than 90%, such as more than 95% or even more than 99%, with SEQ ID NO: 1225, wherein the CDRs are as defined above. In one embodiment, the ISVD specifically binding to PD-1 comprises or consists of the amino acid sequence of SEQ ID NO: 1225.

In another embodiment, the ISVD specifically binding to human PD-1 may have a sequence identity of more than 90%, such as more than 95% or even more than 99%, with SEQ ID NO: 1226, wherein the CDRs are as defined above. In one embodiment, the ISVD specifically binding to PD-1 comprises or consists of the amino acid sequence of SEQ ID NO: 1226.

In another embodiment, the ISVD specifically binding to human PD-1 may have a sequence identity of more than 90%, such as more than 95% or even more than 99%, with SEQ ID NO: 1227, wherein the CDRs are as defined above. In one embodiment, the ISVD specifically binding to PD-1 comprises or consists of the amino acid sequence of SEQ ID NO: 1227.

In another embodiment, the ISVD specifically binding to human PD-1 may have a sequence identity of more than 90%, such as more than 95% or even more than 99%, with SEQ ID NO: 1228, wherein the CDRs are as defined above. In one embodiment, the ISVD specifically binding to PD-1 comprises or consists of the amino acid sequence of SEQ ID NO: 1228.

In another embodiment, the ISVD specifically binding to human PD-1 may have a sequence identity of more than 90%, such as more than 95% or even more than 99%, with SEQ ID NO: 1229, wherein the CDRs are as defined above. In one embodiment, the ISVD specifically binding to PD-1 comprises or consists of the amino acid sequence of SEQ ID NO: 1229.

In another embodiment, the ISVD specifically binding to human PD-1 may have a sequence identity of more than 90%, such as more than 95% or even more than 99%, with SEQ ID NO: 1230, wherein the CDRs are as defined above. In one embodiment, the ISVD specifically binding to PD-1 comprises or consists of the amino acid sequence of SEQ ID NO: 1230.

In another embodiment, the ISVD specifically binding to human PD-1 may have a sequence identity of more than 90%, such as more than 95% or even more than 99%, with SEQ ID NO: 1231, wherein the CDRs are as defined above. In one embodiment, the ISVD specifically binding to PD-1 comprises or consists of the amino acid sequence of SEQ ID NO: 1231.

In another embodiment, the ISVD specifically binding to human PD-1 may have a sequence identity of more than 90%, such as more than 95% or even more than 99%, with SEQ ID NO: 1232, wherein the CDRs are as defined above. In one embodiment, the ISVD specifically binding to PD-1 comprises or consists of the amino acid sequence of SEQ ID NO: 1232.

In another embodiment, the ISVD specifically binding to human PD-1 may have a sequence identity of more than 90%, such as more than 95% or even more than 99%, with SEQ ID NO: 1233, wherein the CDRs are as defined above. In one embodiment, the ISVD specifically binding to PD-1 comprises or consists of the amino acid sequence of SEQ ID NO: 1233.

In another embodiment, the ISVD specifically binding to human PD-1 may have a sequence identity of more than 90%, such as more than 95% or even more than 99%, with SEQ ID NO: 1234, wherein the CDRs are as defined above. In one embodiment, the ISVD specifically binding to PD-1 comprises or consists of the amino acid sequence of SEQ ID NO: 1234.

In another embodiment, the ISVD specifically binding to human PD-1 may have a sequence identity of more than 90%, such as more than 95% or even more than 99%, with SEQ ID NO: 1235, wherein the CDRs are as defined above. In one embodiment, the ISVD specifically binding to PD-1 comprises or consists of the amino acid sequence of SEQ ID NO: 1235.

In another embodiment, the ISVD specifically binding to human PD-1 may have a sequence identity of more than 90%, such as more than 95% or even more than 99%, with SEQ ID NO: 1236, wherein the CDRs are as defined above. In one embodiment, the ISVD specifically binding to PD-1 comprises or consists of the amino acid sequence of SEQ ID NO: 1236.

In another embodiment, the ISVD specifically binding to human PD-1 may have a sequence identity of more than 90%, such as more than 95% or even more than 99%, with SEQ ID NO: 1237, wherein the CDRs are as defined above. In one embodiment, the ISVD specifically binding to PD-1 comprises or consists of the amino acid sequence of SEQ ID NO: 1237.

In another embodiment, the ISVD specifically binding to human PD-1 may have a sequence identity of more than 90%, such as more than 95% or even more than 99%, with SEQ ID NO: 1238, wherein the CDRs are as defined above. In one embodiment, the ISVD specifically binding to PD-1 comprises or consists of the amino acid sequence of SEQ ID NO: 1238.

In another embodiment, the ISVD specifically binding to human PD-1 may have a sequence identity of more than 90%, such as more than 95% or even more than 99%, with SEQ ID NO: 1239, wherein the CDRs are as defined above. In one embodiment, the ISVD specifically binding to PD-1 comprises or consists of the amino acid sequence of SEQ ID NO: 1239.

In another embodiment, the ISVD specifically binding to human PD-1 may have a sequence identity of more than 90%, such as more than 95% or even more than 99%, with SEQ ID NO: 1240, wherein the CDRs are as defined above. In one embodiment, the ISVD specifically binding to PD-1 comprises or consists of the amino acid sequence of SEQ ID NO: 1240.

In another embodiment, the ISVD specifically binding to human PD-1 may have a sequence identity of more than 90%, such as more than 95% or even more than 99%, with SEQ ID NO: 1241, wherein the CDRs are as defined above. In one embodiment, the ISVD specifically binding to PD-1 comprises or consists of the amino acid sequence of SEQ ID NO: 1241.

In another embodiment, the ISVD specifically binding to human PD-1 may have a sequence identity of more than 90%, such as more than 95% or even more than 99%, with SEQ ID NO: 1242, wherein the CDRs are as defined above. In one embodiment, the ISVD specifically binding to PD-1 comprises or consists of the amino acid sequence of SEQ ID NO: 1242.

In another embodiment, the ISVD specifically binding to human PD-1 may have a sequence identity of more than 90%, such as more than 95% or even more than 99%, with SEQ ID NO: 1243, wherein the CDRs are as defined above. In one embodiment, the ISVD specifically binding to PD-1 comprises or consists of the amino acid sequence of SEQ ID NO: 1243.

In another embodiment, the ISVD specifically binding to human PD-1 may have a sequence identity of more than 90%, such as more than 95% or even more than 99%, with SEQ ID NO: 1244, wherein the CDRs are as defined above. In one embodiment, the ISVD specifically binding to PD-1 comprises or consists of the amino acid sequence of SEQ ID NO: 1244.

In another embodiment, the ISVD specifically binding to human PD-1 may have a sequence identity of more than 90%, such as more than 95% or even more than 99%, with SEQ ID NO: 1245, wherein the CDRs are as defined above. In one embodiment, the ISVD specifically binding to PD-1 comprises or consists of the amino acid sequence of SEQ ID NO: 1245.

In another embodiment, the ISVD specifically binding to human PD-1 may have a sequence identity of more than 90%, such as more than 95% or even more than 99%, with SEQ ID NO: 1246, wherein the CDRs are as defined above. In one embodiment, the ISVD specifically binding to PD-1 comprises or consists of the amino acid sequence of SEQ ID NO: 1246.

In another embodiment, the ISVD specifically binding to human PD-1 may have a sequence identity of more than 90%, such as more than 95% or even more than 99%, with SEQ ID NO: 1247, wherein the CDRs are as defined above. In one embodiment, the ISVD specifically binding to PD-1 comprises or consists of the amino acid sequence of SEQ ID NO: 1247.

In another embodiment, the ISVD specifically binding to human PD-1 may have a sequence identity of more than 90%, such as more than 95% or even more than 99%, with SEQ ID NO: 1248, wherein the CDRs are as defined above. In one embodiment, the ISVD specifically binding to PD-1 comprises or consists of the amino acid sequence of SEQ ID NO: 1248.

In another embodiment, the ISVD specifically binding to human PD-1 may have a sequence identity of more than 90%, such as more than 95% or even more than 99%, with SEQ ID NO: 1249, wherein the CDRs are as defined above. In one embodiment, the ISVD specifically binding to PD-1 comprises or consists of the amino acid sequence of SEQ ID NO: 1249.

In another embodiment, the ISVD specifically binding to human PD-1 may have a sequence identity of more than 90%, such as more than 95% or even more than 99%, with SEQ ID NO: 1250, wherein the CDRs are as defined above. In one embodiment, the ISVD specifically binding to PD-1 comprises or consists of the amino acid sequence of SEQ ID NO: 1250.

In another embodiment, the ISVD specifically binding to human PD-1 may have a sequence identity of more than 90%, such as more than 95% or even more than 99%, with SEQ ID NO: 1251, wherein the CDRs are as defined above. In one embodiment, the ISVD specifically binding to PD-1 comprises or consists of the amino acid sequence of SEQ ID NO: 1251.

In another embodiment, the ISVD specifically binding to human PD-1 may have a sequence identity of more than 90%, such as more than 95% or even more than 99%, with SEQ ID NO: 1252, wherein the CDRs are as defined above. In one embodiment, the ISVD specifically binding to PD-1 comprises or consists of the amino acid sequence of SEQ ID NO: 1252.

In another embodiment, the ISVD specifically binding to human PD-1 may have a sequence identity of more than 90%, such as more than 95% or even more than 99%, with SEQ ID NO: 1253, wherein the CDRs are as defined above. In one embodiment, the ISVD specifically binding to PD-1 comprises or consists of the amino acid sequence of SEQ ID NO: 1253.

In another embodiment, the ISVD specifically binding to human PD-1 may have a sequence identity of more than 90%, such as more than 95% or even more than 99%, with SEQ ID NO: 1254, wherein the CDRs are as defined above. In one embodiment, the ISVD specifically binding to PD-1 comprises or consists of the amino acid sequence of SEQ ID NO: 1254.

In another embodiment, the ISVD specifically binding to human PD-1 may have a sequence identity of more than 90%, such as more than 95% or even more than 99%, with SEQ ID NO: 1255, wherein the CDRs are as defined above. In one embodiment, the ISVD specifically binding to PD-1 comprises or consists of the amino acid sequence of SEQ ID NO: 1255.

In another embodiment, the ISVD specifically binding to human PD-1 may have a sequence identity of more than 90%, such as more than 95% or even more than 99%, with SEQ ID NO: 1256, wherein the CDRs are as defined above. In one embodiment, the ISVD specifically binding to PD-1 comprises or consists of the amino acid sequence of SEQ ID NO: 1256.

In another embodiment, the ISVD specifically binding to human PD-1 may have a sequence identity of more than 90%, such as more than 95% or even more than 99%, with SEQ ID NO: 1257, wherein the CDRs are as defined above. In one embodiment, the ISVD specifically binding to PD-1 comprises or consists of the amino acid sequence of SEQ ID NO: 1257.

In another embodiment, the ISVD specifically binding to human PD-1 may have a sequence identity of more than 90%, such as more than 95% or even more than 99%, with SEQ ID NO: 1258, wherein the CDRs are as defined above. In one embodiment, the ISVD specifically binding to PD-1 comprises or consists of the amino acid sequence of SEQ ID NO: 1258.

In another embodiment, the ISVD specifically binding to human PD-1 may have a sequence identity of more than 90%, such as more than 95% or even more than 99%, with SEQ ID NO: 1259, wherein the CDRs are as defined above. In one embodiment, the ISVD specifically binding to PD-1 comprises or consists of the amino acid sequence of SEQ ID NO: 1259.

In another embodiment, the ISVD specifically binding to human PD-1 may have a sequence identity of more than 90%, such as more than 95% or even more than 99%, with SEQ ID NO: 1260, wherein the CDRs are as defined above. In one embodiment, the ISVD specifically binding to PD-1 comprises or consists of the amino acid sequence of SEQ ID NO: 1260.

In another embodiment, the ISVD specifically binding to human PD-1 may have a sequence identity of more than 90%, such as more than 95% or even more than 99%, with SEQ ID NO: 1261, wherein the CDRs are as defined above. In one embodiment, the ISVD specifically binding to PD-1 comprises or consists of the amino acid sequence of SEQ ID NO: 1261.

In another embodiment, the ISVD specifically binding to human PD-1 may have a sequence identity of more than 90%, such as more than 95% or even more than 99%, with SEQ ID NO: 1262, wherein the CDRs are as defined above. In one embodiment, the ISVD specifically binding to PD-1 comprises or consists of the amino acid sequence of SEQ ID NO: 1262.

In another embodiment, the ISVD specifically binding to human PD-1 may have a sequence identity of more than 90%, such as more than 95% or even more than 99%, with SEQ ID NO: 1263, wherein the CDRs are as defined above. In one embodiment, the ISVD specifically binding to PD-1 comprises or consists of the amino acid sequence of SEQ ID NO: 1263.

In another embodiment, the ISVD specifically binding to human PD-1 may have a sequence identity of more than 90%, such as more than 95% or even more than 99%, with SEQ ID NO: 1264, wherein the CDRs are as defined above. In one embodiment, the ISVD specifically binding to PD-1 comprises or consists of the amino acid sequence of SEQ ID NO: 1264.

In another embodiment, the ISVD specifically binding to human PD-1 may have a sequence identity of more than 90%, such as more than 95% or even more than 99%, with SEQ ID NO: 1265, wherein the CDRs are as defined above. In one embodiment, the ISVD specifically binding to PD-1 comprises or consists of the amino acid sequence of SEQ ID NO: 1265.

In another embodiment, the ISVD specifically binding to human PD-1 may have a sequence identity of more than 90%, such as more than 95% or even more than 99%, with SEQ ID NO: 1266, wherein the CDRs are as defined above. In one embodiment, the ISVD specifically binding to PD-1 comprises or consists of the amino acid sequence of SEQ ID NO: 1266.

In another embodiment, the ISVD specifically binding to human PD-1 may have a sequence identity of more than 90%, such as more than 95% or even more than 99%, with SEQ ID NO: 1267, wherein the CDRs are as defined above. In one embodiment, the ISVD specifically binding to PD-1 comprises or consists of the amino acid sequence of SEQ ID NO: 1267.

In another embodiment, the ISVD specifically binding to human PD-1 may have a sequence identity of more than 90%, such as more than 95% or even more than 99%, with SEQ ID NO: 1268, wherein the CDRs are as defined above. In one embodiment, the ISVD specifically binding to PD-1 comprises or consists of the amino acid sequence of SEQ ID NO: 1268.

In another embodiment, the ISVD specifically binding to human PD-1 may have a sequence identity of more than 90%, such as more than 95% or even more than 99%, with SEQ ID NO: 1269, wherein the CDRs are as defined above. In one embodiment, the ISVD specifically binding to PD-1 comprises or consists of the amino acid sequence of SEQ ID NO: 1269.

In another embodiment, the ISVD specifically binding to human PD-1 may have a sequence identity of more than 90%, such as more than 95% or even more than 99%, with SEQ ID NO: 1270, wherein the CDRs are as defined above. In one embodiment, the ISVD specifically binding to PD-1 comprises or consists of the amino acid sequence of SEQ ID NO: 1270.

In another embodiment, the ISVD specifically binding to human PD-1 may have a sequence identity of more than 90%, such as more than 95% or even more than 99%, with SEQ ID NO: 1271, wherein the CDRs are as defined above. In one embodiment, the ISVD specifically binding to PD-1 comprises or consists of the amino acid sequence of SEQ ID NO: 1271.

In another embodiment, the ISVD specifically binding to human PD-1 may have a sequence identity of more than 90%, such as more than 95% or even more than 99%, with SEQ ID NO: 1272, wherein the CDRs are as defined above. In one embodiment, the ISVD specifically binding to PD-1 comprises or consists of the amino acid sequence of SEQ ID NO: 1272.

In another embodiment, the ISVD specifically binding to human PD-1 may have a sequence identity of more than 90%, such as more than 95% or even more than 99%, with SEQ ID NO: 1273, wherein the CDRs are as defined above. In one embodiment, the ISVD specifically binding to PD-1 comprises or consists of the amino acid sequence of SEQ ID NO: 1273.

In another embodiment, the ISVD specifically binding to human PD-1 may have a sequence identity of more than 90%, such as more than 95% or even more than 99%, with SEQ ID NO: 1274, wherein the CDRs are as defined above. In one embodiment, the ISVD specifically binding to PD-1 comprises or consists of the amino acid sequence of SEQ ID NO: 1274.

In another embodiment, the ISVD specifically binding to human PD-1 may have a sequence identity of more than 90%, such as more than 95% or even more than 99%, with SEQ ID NO: 1275, wherein the CDRs are as defined above. In one embodiment, the ISVD specifically binding to PD-1 comprises or consists of the amino acid sequence of SEQ ID NO: 1275.

In another embodiment, the ISVD specifically binding to human PD-1 may have a sequence identity of more than 90%, such as more than 95% or even more than 99%, with SEQ ID NO: 1276, wherein the CDRs are as defined above. In one embodiment, the ISVD specifically binding to PD-1 comprises or consists of the amino acid sequence of SEQ ID NO: 1276.

In another embodiment, the ISVD specifically binding to human PD-1 may have a sequence identity of more than 90%, such as more than 95% or even more than 99%, with SEQ ID NO: 1277, wherein the CDRs are as defined above. In one embodiment, the ISVD specifically binding to PD-1 comprises or consists of the amino acid sequence of SEQ ID NO: 1277.

In another embodiment, the ISVD specifically binding to human PD-1 may have a sequence identity of more than 90%, such as more than 95% or even more than 99%, with SEQ ID NO: 1278, wherein the CDRs are as defined above. In one embodiment, the ISVD specifically binding to PD-1 comprises or consists of the amino acid sequence of SEQ ID NO: 1278.

In another embodiment, the ISVD specifically binding to human PD-1 may have a sequence identity of more than 90%, such as more than 95% or even more than 99%, with SEQ ID NO: 1279, wherein the CDRs are as defined above. In one embodiment, the ISVD specifically binding to PD-1 comprises or consists of the amino acid sequence of SEQ ID NO: 1279.

In another embodiment, the ISVD specifically binding to human PD-1 may have a sequence identity of more than 90%, such as more than 95% or even more than 99%, with SEQ ID NO: 1280, wherein the CDRs are as defined above. In one embodiment, the ISVD specifically binding to PD-1 comprises or consists of the amino acid sequence of SEQ ID NO: 1280.

In another embodiment, the ISVD specifically binding to human PD-1 may have a sequence identity of more than 90%, such as more than 95% or even more than 99%, with SEQ ID NO: 1281, wherein the CDRs are as defined above. In one embodiment, the ISVD specifically binding to PD-1 comprises or consists of the amino acid sequence of SEQ ID NO: 1281.

In another embodiment, the ISVD specifically binding to human PD-1 may have a sequence identity of more than 90%, such as more than 95% or even more than 99%, with SEQ ID NO: 1282, wherein the CDRs are as defined above. In one embodiment, the ISVD specifically binding to PD-1 comprises or consists of the amino acid sequence of SEQ ID NO: 1282.

In another embodiment, the ISVD specifically binding to human PD-1 may have a sequence identity of more than 90%, such as more than 95% or even more than 99%, with SEQ ID NO: 1283, wherein the CDRs are as defined above. In one embodiment, the ISVD specifically binding to PD-1 comprises or consists of the amino acid sequence of SEQ ID NO: 1283.

In another embodiment, the ISVD specifically binding to human PD-1 may have a sequence identity of more than 90%, such as more than 95% or even more than 99%, with SEQ ID NO: 1284, wherein the CDRs are as defined above. In one embodiment, the ISVD specifically binding to PD-1 comprises or consists of the amino acid sequence of SEQ ID NO: 1284.

In another embodiment, the ISVD specifically binding to human PD-1 may have a sequence identity of more than 90%, such as more than 95% or even more than 99%, with SEQ ID NO: 1285, wherein the CDRs are as defined above. In one embodiment, the ISVD specifically binding to PD-1 comprises or consists of the amino acid sequence of SEQ ID NO: 1285.

In another embodiment, the ISVD specifically binding to human PD-1 may have a sequence identity of more than 90%, such as more than 95% or even more than 99%, with SEQ ID NO: 1286, wherein the CDRs are as defined above. In one embodiment, the ISVD specifically binding to PD-1 comprises or consists of the amino acid sequence of SEQ ID NO: 1286.

In another embodiment, the ISVD specifically binding to human PD-1 may have a sequence identity of more than 90%, such as more than 95% or even more than 99%, with SEQ ID NO: 1287, wherein the CDRs are as defined above. In one embodiment, the ISVD specifically binding to PD-1 comprises or consists of the amino acid sequence of SEQ ID NO: 1287.

In another embodiment, the ISVD specifically binding to human PD-1 may have a sequence identity of more than 90%, such as more than 95% or even more than 99%, with SEQ ID NO: 1288, wherein the CDRs are as defined above. In one embodiment, the ISVD specifically binding to PD-1 comprises or consists of the amino acid sequence of SEQ ID NO: 1288.

In another embodiment, the ISVD specifically binding to human PD-1 may have a sequence identity of more than 90%, such as more than 95% or even more than 99%, with SEQ ID NO: 1289, wherein the CDRs are as defined above. In one embodiment, the ISVD specifically binding to PD-1 comprises or consists of the amino acid sequence of SEQ ID NO: 1289.

In another embodiment, the ISVD specifically binding to human PD-1 may have a sequence identity of more than 90%, such as more than 95% or even more than 99%, with SEQ ID NO: 1290, wherein the CDRs are as defined above. In one embodiment, the ISVD specifically binding to PD-1 comprises or consists of the amino acid sequence of SEQ ID NO: 1290.

In another embodiment, the ISVD specifically binding to human PD-1 may have a sequence identity of more than 90%, such as more than 95% or even more than 99%, with SEQ ID NO: 1291, wherein the CDRs are as defined above. In one embodiment, the ISVD specifically binding to PD-1 comprises or consists of the amino acid sequence of SEQ ID NO: 1291.

In another embodiment, the ISVD specifically binding to human PD-1 may have a sequence identity of more than 90%, such as more than 95% or even more than 99%, with SEQ ID NO: 1292, wherein the CDRs are as defined above. In one embodiment, the ISVD specifically binding to PD-1 comprises or consists of the amino acid sequence of SEQ ID NO: 1292.

In another embodiment, the ISVD specifically binding to human PD-1 may have a sequence identity of more than 90%, such as more than 95% or even more than 99%, with SEQ ID NO: 1293, wherein the CDRs are as defined above. In one embodiment, the ISVD specifically binding to PD-1 comprises or consists of the amino acid sequence of SEQ ID NO: 1293.

In another embodiment, the ISVD specifically binding to human PD-1 may have a sequence identity of more than 90%, such as more than 95% or even more than 99%, with SEQ ID NO: 1294, wherein the CDRs are as defined above. In one embodiment, the ISVD specifically binding to PD-1 comprises or consists of the amino acid sequence of SEQ ID NO: 1294.

In another embodiment, the ISVD specifically binding to human PD-1 may have a sequence identity of more than 90%, such as more than 95% or even more than 99%, with SEQ ID NO: 1295, wherein the CDRs are as defined above. In one embodiment, the ISVD specifically binding to PD-1 comprises or consists of the amino acid sequence of SEQ ID NO: 1295.

In another embodiment, the ISVD specifically binding to human PD-1 may have a sequence identity of more than 90%, such as more than 95% or even more than 99%, with SEQ ID NO: 1296, wherein the CDRs are as defined above. In one embodiment, the ISVD specifically binding to PD-1 comprises or consists of the amino acid sequence of SEQ ID NO: 1296.

In another embodiment, the ISVD specifically binding to human PD-1 may have a sequence identity of more than 90%, such as more than 95% or even more than 99%, with SEQ ID NO: 1297, wherein the CDRs are as defined above. In one embodiment, the ISVD specifically binding to PD-1 comprises or consists of the amino acid sequence of SEQ ID NO: 1297.

In another embodiment, the ISVD specifically binding to human PD-1 may have a sequence identity of more than 90%, such as more than 95% or even more than 99%, with SEQ ID NO: 1298, wherein the CDRs are as defined above. In one embodiment, the ISVD specifically binding to PD-1 comprises or consists of the amino acid sequence of SEQ ID NO: 1298.

In another embodiment, the ISVD specifically binding to human PD-1 may have a sequence identity of more than 90%, such as more than 95% or even more than 99%, with SEQ ID NO: 1299, wherein the CDRs are as defined above. In one embodiment, the ISVD specifically binding to PD-1 comprises or consists of the amino acid sequence of SEQ ID NO: 1299.

In another embodiment, the ISVD specifically binding to human PD-1 may have a sequence identity of more than 90%, such as more than 95% or even more than 99%, with SEQ ID NO: 1300, wherein the CDRs are as defined above. In one embodiment, the ISVD specifically binding to PD-1 comprises or consists of the amino acid sequence of SEQ ID NO: 1300.

In another embodiment, the ISVD specifically binding to human PD-1 may have a sequence identity of more than 90%, such as more than 95% or even more than 99%, with SEQ ID NO: 1301, wherein the CDRs are as defined above. In one embodiment, the ISVD specifically binding to PD-1 comprises or consists of the amino acid sequence of SEQ ID NO: 1301.

In another embodiment, the ISVD specifically binding to human PD-1 may have a sequence identity of more than 90%, such as more than 95% or even more than 99%, with SEQ ID NO: 1302, wherein the CDRs are as defined above. In one embodiment, the ISVD specifically binding to PD-1 comprises or consists of the amino acid sequence of SEQ ID NO: 1302.

In another embodiment, the ISVD specifically binding to human PD-1 may have a sequence identity of more than 90%, such as more than 95% or even more than 99%, with SEQ ID NO: 1303, wherein the CDRs are as defined above. In one embodiment, the ISVD specifically binding to PD-1 comprises or consists of the amino acid sequence of SEQ ID NO: 1303.

In another embodiment, the ISVD specifically binding to human PD-1 may have a sequence identity of more than 90%, such as more than 95% or even more than 99%, with SEQ ID NO: 1304, wherein the CDRs are as defined above. In one embodiment, the ISVD specifically binding to PD-1 comprises or consists of the amino acid sequence of SEQ ID NO: 1304.

In another embodiment, the ISVD specifically binding to human PD-1 may have a sequence identity of more than 90%, such as more than 95% or even more than 99%, with SEQ ID NO: 1305, wherein the CDRs are as defined above. In one embodiment, the ISVD specifically binding to PD-1 comprises or consists of the amino acid sequence of SEQ ID NO: 1305.

In another embodiment, the ISVD specifically binding to human PD-1 may have a sequence identity of more than 90%, such as more than 95% or even more than 99%, with SEQ ID NO: 1306, wherein the CDRs are as defined above. In one embodiment, the ISVD specifically binding to PD-1 comprises or consists of the amino acid sequence of SEQ ID NO: 1306.

In another embodiment, the ISVD specifically binding to human PD-1 may have a sequence identity of more than 90%, such as more than 95% or even more than 99%, with SEQ ID NO: 1307, wherein the CDRs are as defined above. In one embodiment, the ISVD specifically binding to PD-1 comprises or consists of the amino acid sequence of SEQ ID NO: 1307.

In another embodiment, the ISVD specifically binding to human PD-1 may have a sequence identity of more than 90%, such as more than 95% or even more than 99%, with SEQ ID NO: 1308, wherein the CDRs are as defined above. In one embodiment, the ISVD specifically binding to PD-1 comprises or consists of the amino acid sequence of SEQ ID NO: 1308.

In another embodiment, the ISVD specifically binding to human PD-1 may have a sequence identity of more than 90%, such as more than 95% or even more than 99%, with SEQ ID NO: 1309, wherein the CDRs are as defined above. In one embodiment, the ISVD specifically binding to PD-1 comprises or consists of the amino acid sequence of SEQ ID NO: 1309.

In another embodiment, the ISVD specifically binding to human PD-1 may have a sequence identity of more than 90%, such as more than 95% or even more than 99%, with SEQ ID NO: 1310, wherein the CDRs are as defined above. In one embodiment, the ISVD specifically binding to PD-1 comprises or consists of the amino acid sequence of SEQ ID NO: 1310.

In another embodiment, the ISVD specifically binding to human PD-1 may have a sequence identity of more than 90%, such as more than 95% or even more than 99%, with SEQ ID NO: 1311, wherein the CDRs are as defined above. In one embodiment, the ISVD specifically binding to PD-1 comprises or consists of the amino acid sequence of SEQ ID NO: 1311.

In another embodiment, the ISVD specifically binding to human PD-1 may have a sequence identity of more than 90%, such as more than 95% or even more than 99%, with SEQ ID NO: 1312, wherein the CDRs are as defined above. In one embodiment, the ISVD specifically binding to PD-1 comprises or consists of the amino acid sequence of SEQ ID NO: 1312.

In another embodiment, the ISVD specifically binding to human PD-1 may have a sequence identity of more than 90%, such as more than 95% or even more than 99%, with SEQ ID NO: 1313, wherein the CDRs are as defined above. In one embodiment, the ISVD specifically binding to PD-1 comprises or consists of the amino acid sequence of SEQ ID NO: 1313.

In another embodiment, the ISVD specifically binding to human PD-1 may have a sequence identity of more than 90%, such as more than 95% or even more than 99%, with SEQ ID NO: 1314, wherein the CDRs are as defined above. In one embodiment, the ISVD specifically binding to PD-1 comprises or consists of the amino acid sequence of SEQ ID NO: 1314.

In another embodiment, the ISVD specifically binding to human PD-1 may have a sequence identity of more than 90%, such as more than 95% or even more than 99%, with SEQ ID NO: 1315, wherein the CDRs are as defined above. In one embodiment, the ISVD specifically binding to PD-1 comprises or consists of the amino acid sequence of SEQ ID NO: 1315.

In another embodiment, the ISVD specifically binding to human PD-1 may have a sequence identity of more than 90%, such as more than 95% or even more than 99%, with SEQ ID NO: 1316, wherein the CDRs are as defined above. In one embodiment, the ISVD specifically binding to PD-1 comprises or consists of the amino acid sequence of SEQ ID NO: 1316.

In another embodiment, the ISVD specifically binding to human PD-1 may have a sequence identity of more than 90%, such as more than 95% or even more than 99%, with SEQ ID NO: 1317, wherein the CDRs are as defined above. In one embodiment, the ISVD specifically binding to PD-1 comprises or consists of the amino acid sequence of SEQ ID NO: 1317.

In another embodiment, the ISVD specifically binding to human PD-1 may have a sequence identity of more than 90%, such as more than 95% or even more than 99%, with SEQ ID NO: 1318, wherein the CDRs are as defined above. In one embodiment, the ISVD specifically binding to PD-1 comprises or consists of the amino acid sequence of SEQ ID NO: 1318.

In another embodiment, the ISVD specifically binding to human PD-1 may have a sequence identity of more than 90%, such as more than 95% or even more than 99%, with SEQ ID NO: 1319, wherein the CDRs are as defined above. In one embodiment, the ISVD specifically binding to PD-1 comprises or consists of the amino acid sequence of SEQ ID NO: 1319.

In another embodiment, the ISVD specifically binding to human PD-1 may have a sequence identity of more than 90%, such as more than 95% or even more than 99%, with SEQ ID NO: 1320, wherein the CDRs are as defined above. In one embodiment, the ISVD specifically binding to PD-1 comprises or consists of the amino acid sequence of SEQ ID NO: 1320.

In another embodiment, the ISVD specifically binding to human PD-1 may have a sequence identity of more than 90%, such as more than 95% or even more than 99%, with SEQ ID NO: 1321, wherein the CDRs are as defined above. In one embodiment, the ISVD specifically binding to PD-1 comprises or consists of the amino acid sequence of SEQ ID NO: 1321.

In another embodiment, the ISVD specifically binding to human PD-1 may have a sequence identity of more than 90%, such as more than 95% or even more than 99%, with SEQ ID NO: 1322, wherein the CDRs are as defined above. In one embodiment, the ISVD specifically binding to PD-1 comprises or consists of the amino acid sequence of SEQ ID NO: 1322.

In another embodiment, the ISVD specifically binding to human PD-1 may have a sequence identity of more than 90%, such as more than 95% or even more than 99%, with SEQ ID NO: 1323, wherein the CDRs are as defined above. In one embodiment, the ISVD specifically binding to PD-1 comprises or consists of the amino acid sequence of SEQ ID NO: 1323.

In another embodiment, the ISVD specifically binding to human PD-1 may have a sequence identity of more than 90%, such as more than 95% or even more than 99%, with SEQ ID NO: 1324, wherein the CDRs are as defined above. In one embodiment, the ISVD specifically binding to PD-1 comprises or consists of the amino acid sequence of SEQ ID NO: 1324.

In another embodiment, the ISVD specifically binding to human PD-1 may have a sequence identity of more than 90%, such as more than 95% or even more than 99%, with SEQ ID NO: 1325, wherein the CDRs are as defined above. In one embodiment, the ISVD specifically binding to PD-1 comprises or consists of the amino acid sequence of SEQ ID NO: 1325.

In another embodiment, the ISVD specifically binding to human PD-1 may have a sequence identity of more than 90%, such as more than 95% or even more than 99%, with SEQ ID NO: 1326, wherein the CDRs are as defined above. In one embodiment, the ISVD specifically binding to PD-1 comprises or consists of the amino acid sequence of SEQ ID NO: 1326.

In another embodiment, the ISVD specifically binding to human PD-1 may have a sequence identity of more than 90%, such as more than 95% or even more than 99%, with SEQ ID NO: 1327, wherein the CDRs are as defined above. In one embodiment, the ISVD specifically binding to PD-1 comprises or consists of the amino acid sequence of SEQ ID NO: 1327.

In another embodiment, the ISVD specifically binding to human PD-1 may have a sequence identity of more than 90%, such as more than 95% or even more than 99%, with SEQ ID NO: 1328, wherein the CDRs are as defined above. In one embodiment, the ISVD specifically binding to PD-1 comprises or consists of the amino acid sequence of SEQ ID NO: 1328.

In another embodiment, the ISVD specifically binding to human PD-1 may have a sequence identity of more than 90%, such as more than 95% or even more than 99%, with SEQ ID NO: 1329, wherein the CDRs are as defined above. In one embodiment, the ISVD specifically binding to PD-1 comprises or consists of the amino acid sequence of SEQ ID NO: 1329.

In another embodiment, the ISVD specifically binding to human PD-1 may have a sequence identity of more than 90%, such as more than 95% or even more than 99%, with SEQ ID NO: 1330, wherein the CDRs are as defined above. In one embodiment, the ISVD specifically binding to PD-1 comprises or consists of the amino acid sequence of SEQ ID NO: 1330.

In another embodiment, the ISVD specifically binding to human PD-1 may have a sequence identity of more than 90%, such as more than 95% or even more than 99%, with SEQ ID NO: 1331, wherein the CDRs are as defined above. In one embodiment, the ISVD specifically binding to PD-1 comprises or consists of the amino acid sequence of SEQ ID NO: 1331.

In another embodiment, the ISVD specifically binding to human PD-1 may have a sequence identity of more than 90%, such as more than 95% or even more than 99%, with SEQ ID NO: 1332, wherein the CDRs are as defined above. In one embodiment, the ISVD specifically binding to PD-1 comprises or consists of the amino acid sequence of SEQ ID NO: 1332.

In another embodiment, the ISVD specifically binding to human PD-1 may have a sequence identity of more than 90%, such as more than 95% or even more than 99%, with SEQ ID NO: 1333, wherein the CDRs are as defined above. In one embodiment, the ISVD specifically binding to PD-1 comprises or consists of the amino acid sequence of SEQ ID NO: 1333.

In another embodiment, the ISVD specifically binding to human PD-1 may have a sequence identity of more than 90%, such as more than 95% or even more than 99%, with SEQ ID NO: 1334, wherein the CDRs are as defined above. In one embodiment, the ISVD specifically binding to PD-1 comprises or consists of the amino acid sequence of SEQ ID NO: 1334.

In another embodiment, the ISVD specifically binding to human PD-1 may have a sequence identity of more than 90%, such as more than 95% or even more than 99%, with SEQ ID NO: 1335, wherein the CDRs are as defined above. In one embodiment, the ISVD specifically binding to PD-1 comprises or consists of the amino acid sequence of SEQ ID NO: 1335.

In another embodiment, the ISVD specifically binding to human PD-1 may have a sequence identity of more than 90%, such as more than 95% or even more than 99%, with SEQ ID NO: 1336, wherein the CDRs are as defined above. In one embodiment, the ISVD specifically binding to PD-1 comprises or consists of the amino acid sequence of SEQ ID NO: 1336.

In another embodiment, the ISVD specifically binding to human PD-1 may have a sequence identity of more than 90%, such as more than 95% or even more than 99%, with SEQ ID NO: 1337, wherein the CDRs are as defined above. In one embodiment, the ISVD specifically binding to PD-1 comprises or consists of the amino acid sequence of SEQ ID NO: 1337.

In another embodiment, the ISVD specifically binding to human PD-1 may have a sequence identity of more than 90%, such as more than 95% or even more than 99%, with SEQ ID NO: 1338, wherein the CDRs are as defined above. In one embodiment, the ISVD specifically binding to PD-1 comprises or consists of the amino acid sequence of SEQ ID NO: 1338.

In another embodiment, the ISVD specifically binding to human PD-1 may have a sequence identity of more than 90%, such as more than 95% or even more than 99%, with SEQ ID NO: 1339, wherein the CDRs are as defined above. In one embodiment, the ISVD specifically binding to PD-1 comprises or consists of the amino acid sequence of SEQ ID NO: 1339.

In another embodiment, the ISVD specifically binding to human PD-1 may have a sequence identity of more than 90%, such as more than 95% or even more than 99%, with SEQ ID NO: 1340, wherein the CDRs are as defined above. In one embodiment, the ISVD specifically binding to PD-1 comprises or consists of the amino acid sequence of SEQ ID NO: 1340.

In another embodiment, the ISVD specifically binding to human PD-1 may have a sequence identity of more than 90%, such as more than 95% or even more than 99%, with SEQ ID NO: 1341, wherein the CDRs are as defined above. In one embodiment, the ISVD specifically binding to PD-1 comprises or consists of the amino acid sequence of SEQ ID NO: 1341.

In another embodiment, the ISVD specifically binding to human PD-1 may have a sequence identity of more than 90%, such as more than 95% or even more than 99%, with SEQ ID NO: 1342, wherein the CDRs are as defined above. In one embodiment, the ISVD specifically binding to PD-1 comprises or consists of the amino acid sequence of SEQ ID NO: 1342.

In another embodiment, the ISVD specifically binding to human PD-1 may have a sequence identity of more than 90%, such as more than 95% or even more than 99%, with SEQ ID NO: 1343, wherein the CDRs are as defined above. In one embodiment, the ISVD specifically binding to PD-1 comprises or consists of the amino acid sequence of SEQ ID NO: 1343.

In another embodiment, the ISVD specifically binding to human PD-1 may have a sequence identity of more than 90%, such as more than 95% or even more than 99%, with SEQ ID NO: 1344, wherein the CDRs are as defined above. In one embodiment, the ISVD specifically binding to PD-1 comprises or consists of the amino acid sequence of SEQ ID NO: 1344.

In another embodiment, the ISVD specifically binding to human PD-1 may have a sequence identity of more than 90%, such as more than 95% or even more than 99%, with SEQ ID NO: 1345, wherein the CDRs are as defined above. In one embodiment, the ISVD specifically binding to PD-1 comprises or consists of the amino acid sequence of SEQ ID NO: 1345.

In another embodiment, the ISVD specifically binding to human PD-1 may have a sequence identity of more than 90%, such as more than 95% or even more than 99%, with SEQ ID NO: 1346, wherein the CDRs are as defined above. In one embodiment, the ISVD specifically binding to PD-1 comprises or consists of the amino acid sequence of SEQ ID NO: 1346.

In another embodiment, the ISVD specifically binding to human PD-1 may have a sequence identity of more than 90%, such as more than 95% or even more than 99%, with SEQ ID NO: 1347, wherein the CDRs are as defined above. In one embodiment, the ISVD specifically binding to PD-1 comprises or consists of the amino acid sequence of SEQ ID NO: 1347.

In another embodiment, the ISVD specifically binding to human PD-1 may have a sequence identity of more than 90%, such as more than 95% or even more than 99%, with SEQ ID NO: 1348, wherein the CDRs are as defined above. In one embodiment, the ISVD specifically binding to PD-1 comprises or consists of the amino acid sequence of SEQ ID NO: 1348.

In another embodiment, the ISVD specifically binding to human PD-1 may have a sequence identity of more than 90%, such as more than 95% or even more than 99%, with SEQ ID NO: 1349, wherein the CDRs are as defined above. In one embodiment, the ISVD specifically binding to PD-1 comprises or consists of the amino acid sequence of SEQ ID NO: 1349.

In another embodiment, the ISVD specifically binding to human PD-1 may have a sequence identity of more than 90%, such as more than 95% or even more than 99%, with SEQ ID NO: 1350, wherein the CDRs are as defined above. In one embodiment, the ISVD specifically binding to PD-1 comprises or consists of the amino acid sequence of SEQ ID NO: 1350.

In another embodiment, the ISVD specifically binding to human PD-1 may have a sequence identity of more than 90%, such as more than 95% or even more than 99%, with SEQ ID NO: 1351, wherein the CDRs are as defined above. In one embodiment, the ISVD specifically binding to PD-1 comprises or consists of the amino acid sequence of SEQ ID NO: 1351.

In another embodiment, the ISVD specifically binding to human PD-1 may have a sequence identity of more than 90%, such as more than 95% or even more than 99%, with SEQ ID NO: 1352, wherein the CDRs are as defined above. In one embodiment, the ISVD specifically binding to PD-1 comprises or consists of the amino acid sequence of SEQ ID NO: 1352.

In another embodiment, the ISVD specifically binding to human PD-1 may have a sequence identity of more than 90%, such as more than 95% or even more than 99%, with SEQ ID NO: 1353, wherein the CDRs are as defined above. In one embodiment, the ISVD specifically binding to PD-1 comprises or consists of the amino acid sequence of SEQ ID NO: 1353.

In another embodiment, the ISVD specifically binding to human PD-1 may have a sequence identity of more than 90%, such as more than 95% or even more than 99%, with SEQ ID NO: 1354, wherein the CDRs are as defined above. In one embodiment, the ISVD specifically binding to PD-1 comprises or consists of the amino acid sequence of SEQ ID NO: 1354.

In another embodiment, the ISVD specifically binding to human PD-1 may have a sequence identity of more than 90%, such as more than 95% or even more than 99%, with SEQ ID NO: 1355, wherein the CDRs are as defined above. In one embodiment, the ISVD specifically binding to PD-1 comprises or consists of the amino acid sequence of SEQ ID NO: 1355.

In another embodiment, the ISVD specifically binding to human PD-1 may have a sequence identity of more than 90%, such as more than 95% or even more than 99%, with SEQ ID NO: 1356, wherein the CDRs are as defined above. In one embodiment, the ISVD specifically binding to PD-1 comprises or consists of the amino acid sequence of SEQ ID NO: 1356.

In another embodiment, the ISVD specifically binding to human PD-1 may have a sequence identity of more than 90%, such as more than 95% or even more than 99%, with SEQ ID NO: 1357, wherein the CDRs are as defined above. In one embodiment, the ISVD specifically binding to PD-1 comprises or consists of the amino acid sequence of SEQ ID NO: 1357.

In another embodiment, the ISVD specifically binding to human PD-1 may have a sequence identity of more than 90%, such as more than 95% or even more than 99%, with SEQ ID NO: 1358, wherein the CDRs are as defined above. In one embodiment, the ISVD specifically binding to PD-1 comprises or consists of the amino acid sequence of SEQ ID NO: 1358.

In another embodiment, the ISVD specifically binding to human PD-1 may have a sequence identity of more than 90%, such as more than 95% or even more than 99%, with SEQ ID NO: 1359, wherein the CDRs are as defined above. In one embodiment, the ISVD specifically binding to PD-1 comprises or consists of the amino acid sequence of SEQ ID NO: 1359.

In another embodiment, the ISVD specifically binding to human PD-1 may have a sequence identity of more than 90%, such as more than 95% or even more than 99%, with SEQ ID NO: 1360, wherein the CDRs are as defined above. In one embodiment, the ISVD specifically binding to PD-1 comprises or consists of the amino acid sequence of SEQ ID NO: 1360.

In another embodiment, the ISVD specifically binding to human PD-1 may have a sequence identity of more than 90%, such as more than 95% or even more than 99%, with SEQ ID NO: 1361, wherein the CDRs are as defined above. In one embodiment, the ISVD specifically binding to PD-1 comprises or consists of the amino acid sequence of SEQ ID NO: 1361.

In another embodiment, the ISVD specifically binding to human PD-1 may have a sequence identity of more than 90%, such as more than 95% or even more than 99%, with SEQ ID NO: 1362, wherein the CDRs are as defined above. In one embodiment, the ISVD specifically binding to PD-1 comprises or consists of the amino acid sequence of SEQ ID NO: 1362.

In another embodiment, the ISVD specifically binding to human PD-1 may have a sequence identity of more than 90%, such as more than 95% or even more than 99%, with SEQ ID NO: 1363, wherein the CDRs are as defined above. In one embodiment, the ISVD specifically binding to PD-1 comprises or consists of the amino acid sequence of SEQ ID NO: 1363.

In another embodiment, the ISVD specifically binding to human PD-1 may have a sequence identity of more than 90%, such as more than 95% or even more than 99%, with SEQ ID NO: 1364, wherein the CDRs are as defined above. In one embodiment, the ISVD specifically binding to PD-1 comprises or consists of the amino acid sequence of SEQ ID NO: 1364.

In another embodiment, the ISVD specifically binding to human PD-1 may have a sequence identity of more than 90%, such as more than 95% or even more than 99%, with SEQ ID NO: 1365, wherein the CDRs are as defined above. In one embodiment, the ISVD specifically binding to PD-1 comprises or consists of the amino acid sequence of SEQ ID NO: 1365.

In another embodiment, the ISVD specifically binding to human PD-1 may have a sequence identity of more than 90%, such as more than 95% or even more than 99%, with SEQ ID NO: 1366, wherein the CDRs are as defined above. In one embodiment, the ISVD specifically binding to PD-1 comprises or consists of the amino acid sequence of SEQ ID NO: 1366.

In another embodiment, the ISVD specifically binding to human PD-1 may have a sequence identity of more than 90%, such as more than 95% or even more than 99%, with SEQ ID NO: 1367, wherein the CDRs are as defined above. In one embodiment, the ISVD specifically binding to PD-1 comprises or consists of the amino acid sequence of SEQ ID NO: 1367.

In another embodiment, the ISVD specifically binding to human PD-1 may have a sequence identity of more than 90%, such as more than 95% or even more than 99%, with SEQ ID NO: 1368, wherein the CDRs are as defined above. In one embodiment, the ISVD specifically binding to PD-1 comprises or consists of the amino acid sequence of SEQ ID NO: 1368.

In another embodiment, the ISVD specifically binding to human PD-1 may have a sequence identity of more than 90%, such as more than 95% or even more than 99%, with SEQ ID NO: 1369, wherein the CDRs are as defined above. In one embodiment, the ISVD specifically binding to PD-1 comprises or consists of the amino acid sequence of SEQ ID NO: 1369.

In another embodiment, the ISVD specifically binding to human PD-1 may have a sequence identity of more than 90%, such as more than 95% or even more than 99%, with SEQ ID NO: 1370, wherein the CDRs are as defined above. In one embodiment, the ISVD specifically binding to PD-1 comprises or consists of the amino acid sequence of SEQ ID NO: 1370.

In another embodiment, the ISVD specifically binding to human PD-1 may have a sequence identity of more than 90%, such as more than 95% or even more than 99%, with SEQ ID NO: 1371, wherein the CDRs are as defined above. In one embodiment, the ISVD specifically binding to PD-1 comprises or consists of the amino acid sequence of SEQ ID NO: 1371.

In another embodiment, the ISVD specifically binding to human PD-1 may have a sequence identity of more than 90%, such as more than 95% or even more than 99%, with SEQ ID NO: 1372, wherein the CDRs are as defined above. In one embodiment, the ISVD specifically binding to PD-1 comprises or consists of the amino acid sequence of SEQ ID NO: 1372.

In another embodiment, the ISVD specifically binding to human PD-1 may have a sequence identity of more than 90%, such as more than 95% or even more than 99%, with SEQ ID NO: 1373, wherein the CDRs are as defined above. In one embodiment, the ISVD specifically binding to PD-1 comprises or consists of the amino acid sequence of SEQ ID NO: 1373.

In another embodiment, the ISVD specifically binding to human PD-1 may have a sequence identity of more than 90%, such as more than 95% or even more than 99%, with SEQ ID NO: 1374, wherein the CDRs are as defined above. In one embodiment, the ISVD specifically binding to PD-1 comprises or consists of the amino acid sequence of SEQ ID NO: 1374.

In another embodiment, the ISVD specifically binding to human PD-1 may have a sequence identity of more than 90%, such as more than 95% or even more than 99%, with SEQ ID NO: 1375, wherein the CDRs are as defined above. In one embodiment, the ISVD specifically binding to PD-1 comprises or consists of the amino acid sequence of SEQ ID NO: 1375.

In another embodiment, the ISVD specifically binding to human PD-1 may have a sequence identity of more than 90%, such as more than 95% or even more than 99%, with SEQ ID NO: 1376, wherein the CDRs are as defined above. In one embodiment, the ISVD specifically binding to PD-1 comprises or consists of the amino acid sequence of SEQ ID NO: 1376.

In another embodiment, the ISVD specifically binding to human PD-1 may have a sequence identity of more than 90%, such as more than 95% or even more than 99%, with SEQ ID NO: 1377, wherein the CDRs are as defined above. In one embodiment, the ISVD specifically binding to PD-1 comprises or consists of the amino acid sequence of SEQ ID NO: 1377.

In another embodiment, the ISVD specifically binding to human PD-1 may have a sequence identity of more than 90%, such as more than 95% or even more than 99%, with SEQ ID NO: 1378, wherein the CDRs are as defined above. In one embodiment, the ISVD specifically binding to PD-1 comprises or consists of the amino acid sequence of SEQ ID NO: 1378.

In another embodiment, the ISVD specifically binding to human PD-1 may have a sequence identity of more than 90%, such as more than 95% or even more than 99%, with SEQ ID NO: 1379, wherein the CDRs are as defined above. In one embodiment, the ISVD specifically binding to PD-1 comprises or consists of the amino acid sequence of SEQ ID NO: 1379.

In another embodiment, the ISVD specifically binding to human PD-1 may have a sequence identity of more than 90%, such as more than 95% or even more than 99%, with SEQ ID NO: 1380, wherein the CDRs are as defined above. In one embodiment, the ISVD specifically binding to PD-1 comprises or consists of the amino acid sequence of SEQ ID NO: 1380.

In another embodiment, the ISVD specifically binding to human PD-1 may have a sequence identity of more than 90%, such as more than 95% or even more than 99%, with SEQ ID NO: 1381, wherein the CDRs are as defined above. In one embodiment, the ISVD specifically binding to PD-1 comprises or consists of the amino acid sequence of SEQ ID NO: 1381.

In another embodiment, the ISVD specifically binding to human PD-1 may have a sequence identity of more than 90%, such as more than 95% or even more than 99%, with SEQ ID NO: 1382, wherein the CDRs are as defined above. In one embodiment, the ISVD specifically binding to PD-1 comprises or consists of the amino acid sequence of SEQ ID NO: 1382.

In another embodiment, the ISVD specifically binding to human PD-1 may have a sequence identity of more than 90%, such as more than 95% or even more than 99%, with SEQ ID NO: 1383, wherein the CDRs are as defined above. In one embodiment, the ISVD specifically binding to PD-1 comprises or consists of the amino acid sequence of SEQ ID NO: 1383.

In another embodiment, the ISVD specifically binding to human PD-1 may have a sequence identity of more than 90%, such as more than 95% or even more than 99%, with SEQ ID NO: 1384, wherein the CDRs are as defined above. In one embodiment, the ISVD specifically binding to PD-1 comprises or consists of the amino acid sequence of SEQ ID NO: 1384.

In another embodiment, the ISVD specifically binding to human PD-1 may have a sequence identity of more than 90%, such as more than 95% or even more than 99%, with SEQ ID NO: 1385, wherein the CDRs are as defined above. In one embodiment, the ISVD specifically binding to PD-1 comprises or consists of the amino acid sequence of SEQ ID NO: 1385.

In another embodiment, the ISVD specifically binding to human PD-1 may have a sequence identity of more than 90%, such as more than 95% or even more than 99%, with SEQ ID NO: 1386, wherein the CDRs are as defined above. In one embodiment, the ISVD specifically binding to PD-1 comprises or consists of the amino acid sequence of SEQ ID NO: 1386.

In another embodiment, the ISVD specifically binding to human PD-1 may have a sequence identity of more than 90%, such as more than 95% or even more than 99%, with SEQ ID NO: 1387, wherein the CDRs are as defined above. In one embodiment, the ISVD specifically binding to PD-1 comprises or consists of the amino acid sequence of SEQ ID NO: 1387.

In another embodiment, the ISVD specifically binding to human PD-1 may have a sequence identity of more than 90%, such as more than 95% or even more than 99%, with SEQ ID NO: 1388, wherein the CDRs are as defined above. In one embodiment, the ISVD specifically binding to PD-1 comprises or consists of the amino acid sequence of SEQ ID NO: 1388.

In another embodiment, the ISVD specifically binding to human PD-1 may have a sequence identity of more than 90%, such as more than 95% or even more than 99%, with SEQ ID NO: 1389, wherein the CDRs are as defined above. In one embodiment, the ISVD specifically binding to PD-1 comprises or consists of the amino acid sequence of SEQ ID NO: 1389.

In another embodiment, the ISVD specifically binding to human PD-1 may have a sequence identity of more than 90%, such as more than 95% or even more than 99%, with SEQ ID NO: 1390, wherein the CDRs are as defined above. In one embodiment, the ISVD specifically binding to PD-1 comprises or consists of the amino acid sequence of SEQ ID NO: 1390.

In another embodiment, the ISVD specifically binding to human PD-1 may have a sequence identity of more than 90%, such as more than 95% or even more than 99%, with SEQ ID NO: 1391, wherein the CDRs are as defined above. In one embodiment, the ISVD specifically binding to PD-1 comprises or consists of the amino acid sequence of SEQ ID NO: 1391.

In another embodiment, the ISVD specifically binding to human PD-1 may have a sequence identity of more than 90%, such as more than 95% or even more than 99%, with SEQ ID NO: 1392, wherein the CDRs are as defined above. In one embodiment, the ISVD specifically binding to PD-1 comprises or consists of the amino acid sequence of SEQ ID NO: 1392.

In another embodiment, the ISVD specifically binding to human PD-1 may have a sequence identity of more than 90%, such as more than 95% or even more than 99%, with SEQ ID NO: 1393, wherein the CDRs are as defined above. In one embodiment, the ISVD specifically binding to PD-1 comprises or consists of the amino acid sequence of SEQ ID NO: 1393.

In another embodiment, the ISVD specifically binding to human PD-1 may have a sequence identity of more than 90%, such as more than 95% or even more than 99%, with SEQ ID NO: 1394, wherein the CDRs are as defined above. In one embodiment, the ISVD specifically binding to PD-1 comprises or consists of the amino acid sequence of SEQ ID NO: 1394.

In another embodiment, the ISVD specifically binding to human PD-1 may have a sequence identity of more than 90%, such as more than 95% or even more than 99%, with SEQ ID NO: 1395, wherein the CDRs are as defined above. In one embodiment, the ISVD specifically binding to PD-1 comprises or consists of the amino acid sequence of SEQ ID NO: 1395.

In another embodiment, the ISVD specifically binding to human PD-1 may have a sequence identity of more than 90%, such as more than 95% or even more than 99%, with SEQ ID NO: 1396, wherein the CDRs are as defined above. In one embodiment, the ISVD specifically binding to PD-1 comprises or consists of the amino acid sequence of SEQ ID NO: 1396.

In another embodiment, the ISVD specifically binding to human PD-1 may have a sequence identity of more than 90%, such as more than 95% or even more than 99%, with SEQ ID NO: 1397, wherein the CDRs are as defined above. In one embodiment, the ISVD specifically binding to PD-1 comprises or consists of the amino acid sequence of SEQ ID NO: 1397.

In another embodiment, the ISVD specifically binding to human PD-1 may have a sequence identity of more than 90%, such as more than 95% or even more than 99%, with SEQ ID NO: 1398, wherein the CDRs are as defined above. In one embodiment, the ISVD specifically binding to PD-1 comprises or consists of the amino acid sequence of SEQ ID NO: 1398.

In another embodiment, the ISVD specifically binding to human PD-1 may have a sequence identity of more than 90%, such as more than 95% or even more than 99%, with SEQ ID NO: 1399, wherein the CDRs are as defined above. In one embodiment, the ISVD specifically binding to PD-1 comprises or consists of the amino acid sequence of SEQ ID NO: 1399.

In another embodiment, the ISVD specifically binding to human PD-1 may have a sequence identity of more than 90%, such as more than 95% or even more than 99%, with SEQ ID NO: 1400, wherein the CDRs are as defined above. In one embodiment, the ISVD specifically binding to PD-1 comprises or consists of the amino acid sequence of SEQ ID NO: 1400.

In another embodiment, the ISVD specifically binding to human PD-1 may have a sequence identity of more than 90%, such as more than 95% or even more than 99%, with SEQ ID NO: 1401, wherein the CDRs are as defined above. In one embodiment, the ISVD specifically binding to PD-1 comprises or consists of the amino acid sequence of SEQ ID NO: 1401.

In another embodiment, the ISVD specifically binding to human PD-1 may have a sequence identity of more than 90%, such as more than 95% or even more than 99%, with SEQ ID NO: 1402, wherein the CDRs are as defined above. In one embodiment, the ISVD specifically binding to PD-1 comprises or consists of the amino acid sequence of SEQ ID NO: 1402.

In another embodiment, the ISVD specifically binding to human PD-1 may have a sequence identity of more than 90%, such as more than 95% or even more than 99%, with SEQ ID NO: 1403, wherein the CDRs are as defined above. In one embodiment, the ISVD specifically binding to PD-1 comprises or consists of the amino acid sequence of SEQ ID NO: 1403.

In another embodiment, the ISVD specifically binding to human PD-1 may have a sequence identity of more than 90%, such as more than 95% or even more than 99%, with SEQ ID NO: 1404, wherein the CDRs are as defined above. In one embodiment, the ISVD specifically binding to PD-1 comprises or consists of the amino acid sequence of SEQ ID NO: 1404.

In another embodiment, the ISVD specifically binding to human PD-1 may have a sequence identity of more than 90%, such as more than 95% or even more than 99%, with SEQ ID NO: 1405, wherein the CDRs are as defined above. In one embodiment, the ISVD specifically binding to PD-1 comprises or consists of the amino acid sequence of SEQ ID NO: 1405.

In another embodiment, the ISVD specifically binding to human PD-1 may have a sequence identity of more than 90%, such as more than 95% or even more than 99%, with SEQ ID NO: 1406, wherein the CDRs are as defined above. In one embodiment, the ISVD specifically binding to PD-1 comprises or consists of the amino acid sequence of SEQ ID NO: 1406.

In another embodiment, the ISVD specifically binding to human PD-1 may have a sequence identity of more than 90%, such as more than 95% or even more than 99%, with SEQ ID NO: 1407, wherein the CDRs are as defined above. In one embodiment, the ISVD specifically binding to PD-1 comprises or consists of the amino acid sequence of SEQ ID NO: 1407.

In another embodiment, the ISVD specifically binding to human PD-1 may have a sequence identity of more than 90%, such as more than 95% or even more than 99%, with SEQ ID NO: 1408, wherein the CDRs are as defined above. In one embodiment, the ISVD specifically binding to PD-1 comprises or consists of the amino acid sequence of SEQ ID NO: 1408.

In another embodiment, the ISVD specifically binding to human PD-1 may have a sequence identity of more than 90%, such as more than 95% or even more than 99%, with SEQ ID NO: 1409, wherein the CDRs are as defined above. In one embodiment, the ISVD specifically binding to PD-1 comprises or consists of the amino acid sequence of SEQ ID NO: 1409.

In another embodiment, the ISVD specifically binding to human PD-1 may have a sequence identity of more than 90%, such as more than 95% or even more than 99%, with SEQ ID NO: 1410, wherein the CDRs are as defined above. In one embodiment, the ISVD specifically binding to PD-1 comprises or consists of the amino acid sequence of SEQ ID NO: 1410.

In another embodiment, the ISVD specifically binding to human PD-1 may have a sequence identity of more than 90%, such as more than 95% or even more than 99%, with SEQ ID NO: 1411, wherein the CDRs are as defined above. In one embodiment, the ISVD specifically binding to PD-1 comprises or consists of the amino acid sequence of SEQ ID NO: 1411.

In another embodiment, the ISVD specifically binding to human PD-1 may have a sequence identity of more than 90%, such as more than 95% or even more than 99%, with SEQ ID NO: 1412, wherein the CDRs are as defined above. In one embodiment, the ISVD specifically binding to PD-1 comprises or consists of the amino acid sequence of SEQ ID NO: 1412.

In another embodiment, the ISVD specifically binding to human PD-1 may have a sequence identity of more than 90%, such as more than 95% or even more than 99%, with SEQ ID NO: 1413, wherein the CDRs are as defined above. In one embodiment, the ISVD specifically binding to PD-1 comprises or consists of the amino acid sequence of SEQ ID NO: 1413.

In another embodiment, the ISVD specifically binding to human PD-1 may have a sequence identity of more than 90%, such as more than 95% or even more than 99%, with SEQ ID NO: 1414, wherein the CDRs are as defined above. In one embodiment, the ISVD specifically binding to PD-1 comprises or consists of the amino acid sequence of SEQ ID NO: 1414.

In another embodiment, the ISVD specifically binding to human PD-1 may have a sequence identity of more than 90%, such as more than 95% or even more than 99%, with SEQ ID NO: 1415, wherein the CDRs are as defined above. In one embodiment, the ISVD specifically binding to PD-1 comprises or consists of the amino acid sequence of SEQ ID NO: 1415.

In another embodiment, the ISVD specifically binding to human PD-1 may have a sequence identity of more than 90%, such as more than 95% or even more than 99%, with SEQ ID NO: 1416, wherein the CDRs are as defined above. In one embodiment, the ISVD specifically binding to PD-1 comprises or consists of the amino acid sequence of SEQ ID NO: 1416.

In another embodiment, the ISVD specifically binding to human PD-1 may have a sequence identity of more than 90%, such as more than 95% or even more than 99%, with SEQ ID NO: 1417, wherein the CDRs are as defined above. In one embodiment, the ISVD specifically binding to PD-1 comprises or consists of the amino acid sequence of SEQ ID NO: 1417.

In another embodiment, the ISVD specifically binding to human PD-1 may have a sequence identity of more than 90%, such as more than 95% or even more than 99%, with SEQ ID NO: 1418, wherein the CDRs are as defined above. In one embodiment, the ISVD specifically binding to PD-1 comprises or consists of the amino acid sequence of SEQ ID NO: 1418.

In another embodiment, the ISVD specifically binding to human PD-1 may have a sequence identity of more than 90%, such as more than 95% or even more than 99%, with SEQ ID NO: 1419, wherein the CDRs are as defined above. In one embodiment, the ISVD specifically binding to PD-1 comprises or consists of the amino acid sequence of SEQ ID NO: 1419.

In another embodiment, the ISVD specifically binding to human PD-1 may have a sequence identity of more than 90%, such as more than 95% or even more than 99%, with SEQ ID NO: 1420, wherein the CDRs are as defined above. In one embodiment, the ISVD specifically binding to PD-1 comprises or consists of the amino acid sequence of SEQ ID NO: 1420.

In another embodiment, the ISVD specifically binding to human PD-1 may have a sequence identity of more than 90%, such as more than 95% or even more than 99%, with SEQ ID NO: 1421, wherein the CDRs are as defined above. In one embodiment, the ISVD specifically binding to PD-1 comprises or consists of the amino acid sequence of SEQ ID NO: 1421.

In another embodiment, the ISVD specifically binding to human PD-1 may have a sequence identity of more than 90%, such as more than 95% or even more than 99%, with SEQ ID NO: 1422, wherein the CDRs are as defined above. In one embodiment, the ISVD specifically binding to PD-1 comprises or consists of the amino acid sequence of SEQ ID NO: 1422.

In another embodiment, the ISVD specifically binding to human PD-1 may have a sequence identity of more than 90%, such as more than 95% or even more than 99%, with SEQ ID NO: 1423, wherein the CDRs are as defined above. In one embodiment, the ISVD specifically binding to PD-1 comprises or consists of the amino acid sequence of SEQ ID NO: 1423.

In another embodiment, the ISVD specifically binding to human PD-1 may have a sequence identity of more than 90%, such as more than 95% or even more than 99%, with SEQ ID NO: 1424, wherein the CDRs are as defined above. In one embodiment, the ISVD specifically binding to PD-1 comprises or consists of the amino acid sequence of SEQ ID NO: 1424.

In another embodiment, the ISVD specifically binding to human PD-1 may have a sequence identity of more than 90%, such as more than 95% or even more than 99%, with SEQ ID NO: 1425, wherein the CDRs are as defined above. In one embodiment, the ISVD specifically binding to PD-1 comprises or consists of the amino acid sequence of SEQ ID NO: 1425.

In another embodiment, the ISVD specifically binding to human PD-1 may have a sequence identity of more than 90%, such as more than 95% or even more than 99%, with SEQ ID NO: 1426, wherein the CDRs are as defined above. In one embodiment, the ISVD specifically binding to PD-1 comprises or consists of the amino acid sequence of SEQ ID NO: 1426.

In another embodiment, the ISVD specifically binding to human PD-1 may have a sequence identity of more than 90%, such as more than 95% or even more than 99%, with SEQ ID NO: 1427, wherein the CDRs are as defined above. In one embodiment, the ISVD specifically binding to PD-1 comprises or consists of the amino acid sequence of SEQ ID NO: 1427.

In another embodiment, the ISVD specifically binding to human PD-1 may have a sequence identity of more than 90%, such as more than 95% or even more than 99%, with SEQ ID NO: 1428, wherein the CDRs are as defined above. In one embodiment, the ISVD specifically binding to PD-1 comprises or consists of the amino acid sequence of SEQ ID NO: 1428.

In another embodiment, the ISVD specifically binding to human PD-1 may have a sequence identity of more than 90%, such as more than 95% or even more than 99%, with SEQ ID NO: 1429, wherein the CDRs are as defined above. In one embodiment, the ISVD specifically binding to PD-1 comprises or consists of the amino acid sequence of SEQ ID NO: 1429.

In another embodiment, the ISVD specifically binding to human PD-1 may have a sequence identity of more than 90%, such as more than 95% or even more than 99%, with SEQ ID NO: 1430, wherein the CDRs are as defined above. In one embodiment, the ISVD specifically binding to PD-1 comprises or consists of the amino acid sequence of SEQ ID NO: 1430.

In another embodiment, the ISVD specifically binding to human PD-1 may have a sequence identity of more than 90%, such as more than 95% or even more than 99%, with SEQ ID NO: 1431, wherein the CDRs are as defined above. In one embodiment, the ISVD specifically binding to PD-1 comprises or consists of the amino acid sequence of SEQ ID NO: 1431.

In another embodiment, the ISVD specifically binding to human PD-1 may have a sequence identity of more than 90%, such as more than 95% or even more than 99%, with SEQ ID NO: 1432, wherein the CDRs are as defined above. In one embodiment, the ISVD specifically binding to PD-1 comprises or consists of the amino acid sequence of SEQ ID NO: 1432.

In another embodiment, the ISVD specifically binding to human PD-1 may have a sequence identity of more than 90%, such as more than 95% or even more than 99%, with SEQ ID NO: 1433, wherein the CDRs are as defined above. In one embodiment, the ISVD specifically binding to PD-1 comprises or consists of the amino acid sequence of SEQ ID NO: 1433.

In another embodiment, the ISVD specifically binding to human PD-1 may have a sequence identity of more than 90%, such as more than 95% or even more than 99%, with SEQ ID NO: 1434, wherein the CDRs are as defined above. In one embodiment, the ISVD specifically binding to PD-1 comprises or consists of the amino acid sequence of SEQ ID NO: 1434.

In another embodiment, the ISVD specifically binding to human PD-1 may have a sequence identity of more than 90%, such as more than 95% or even more than 99%, with SEQ ID NO: 1435, wherein the CDRs are as defined above. In one embodiment, the ISVD specifically binding to PD-1 comprises or consists of the amino acid sequence of SEQ ID NO: 1435.

In another embodiment, the ISVD specifically binding to human PD-1 may have a sequence identity of more than 90%, such as more than 95% or even more than 99%, with SEQ ID NO: 1436, wherein the CDRs are as defined above. In one embodiment, the ISVD specifically binding to PD-1 comprises or consists of the amino acid sequence of SEQ ID NO: 1436.

In another embodiment, the ISVD specifically binding to human PD-1 may have a sequence identity of more than 90%, such as more than 95% or even more than 99%, with SEQ ID NO: 1437, wherein the CDRs are as defined above. In one embodiment, the ISVD specifically binding to PD-1 comprises or consists of the amino acid sequence of SEQ ID NO: 1437.

In another embodiment, the ISVD specifically binding to human PD-1 may have a sequence identity of more than 90%, such as more than 95% or even more than 99%, with SEQ ID NO: 1438, wherein the CDRs are as defined above. In one embodiment, the ISVD specifically binding to PD-1 comprises or consists of the amino acid sequence of SEQ ID NO: 1438.

In another embodiment, the ISVD specifically binding to human PD-1 may have a sequence identity of more than 90%, such as more than 95% or even more than 99%, with SEQ ID NO: 1439, wherein the CDRs are as defined above. In one embodiment, the ISVD specifically binding to PD-1 comprises or consists of the amino acid sequence of SEQ ID NO: 1439.

In another embodiment, the ISVD specifically binding to human PD-1 may have a sequence identity of more than 90%, such as more than 95% or even more than 99%, with SEQ ID NO: 1440, wherein the CDRs are as defined above. In one embodiment, the ISVD specifically binding to PD-1 comprises or consists of the amino acid sequence of SEQ ID NO: 1440.

In another embodiment, the ISVD specifically binding to human PD-1 may have a sequence identity of more than 90%, such as more than 95% or even more than 99%, with SEQ ID NO: 1441, wherein the CDRs are as defined above. In one embodiment, the ISVD specifically binding to PD-1 comprises or consists of the amino acid sequence of SEQ ID NO: 1441.

In another embodiment, the ISVD specifically binding to human PD-1 may have a sequence identity of more than 90%, such as more than 95% or even more than 99%, with SEQ ID NO: 1442, wherein the CDRs are as defined above. In one embodiment, the ISVD specifically binding to PD-1 comprises or consists of the amino acid sequence of SEQ ID NO: 1442.

In another embodiment, the ISVD specifically binding to human PD-1 may have a sequence identity of more than 90%, such as more than 95% or even more than 99%, with SEQ ID NO: 1443, wherein the CDRs are as defined above. In one embodiment, the ISVD specifically binding to PD-1 comprises or consists of the amino acid sequence of SEQ ID NO: 1443.

In another embodiment, the ISVD specifically binding to human PD-1 may have a sequence identity of more than 90%, such as more than 95% or even more than 99%, with SEQ ID NO: 1444, wherein the CDRs are as defined above. In one embodiment, the ISVD specifically binding to PD-1 comprises or consists of the amino acid sequence of SEQ ID NO: 1444.

In another embodiment, the ISVD specifically binding to human PD-1 may have a sequence identity of more than 90%, such as more than 95% or even more than 99%, with SEQ ID NO: 1445, wherein the CDRs are as defined above. In one embodiment, the ISVD specifically binding to PD-1 comprises or consists of the amino acid sequence of SEQ ID NO: 1445.

In another embodiment, the ISVD specifically binding to human PD-1 may have a sequence identity of more than 90%, such as more than 95% or even more than 99%, with SEQ ID NO: 1446, wherein the CDRs are as defined above. In one embodiment, the ISVD specifically binding to PD-1 comprises or consists of the amino acid sequence of SEQ ID NO: 1446.

In another embodiment, the ISVD specifically binding to human PD-1 may have a sequence identity of more than 90%, such as more than 95% or even more than 99%, with SEQ ID NO: 1447, wherein the CDRs are as defined above. In one embodiment, the ISVD specifically binding to PD-1 comprises or consists of the amino acid sequence of SEQ ID NO: 1447.

In another embodiment, the ISVD specifically binding to human PD-1 may have a sequence identity of more than 90%, such as more than 95% or even more than 99%, with SEQ ID NO: 1448, wherein the CDRs are as defined above. In one embodiment, the ISVD specifically binding to PD-1 comprises or consists of the amino acid sequence of SEQ ID NO: 1448.

In another embodiment, the ISVD specifically binding to human PD-1 may have a sequence identity of more than 90%, such as more than 95% or even more than 99%, with SEQ ID NO: 1449, wherein the CDRs are as defined above. In one embodiment, the ISVD specifically binding to PD-1 comprises or consists of the amino acid sequence of SEQ ID NO: 1449.

In another embodiment, the ISVD specifically binding to human PD-1 may have a sequence identity of more than 90%, such as more than 95% or even more than 99%, with SEQ ID NO: 1450, wherein the CDRs are as defined above. In one embodiment, the ISVD specifically binding to PD-1 comprises or consists of the amino acid sequence of SEQ ID NO: 1450.

In another embodiment, the ISVD specifically binding to human PD-1 may have a sequence identity of more than 90%, such as more than 95% or even more than 99%, with SEQ ID NO: 1451, wherein the CDRs are as defined above. In one embodiment, the ISVD specifically binding to PD-1 comprises or consists of the amino acid sequence of SEQ ID NO: 1451.

In another embodiment, the ISVD specifically binding to human PD-1 may have a sequence identity of more than 90%, such as more than 95% or even more than 99%, with SEQ ID NO: 1452, wherein the CDRs are as defined above. In one embodiment, the ISVD specifically binding to PD-1 comprises or consists of the amino acid sequence of SEQ ID NO: 1452.

In another embodiment, the ISVD specifically binding to human PD-1 may have a sequence identity of more than 90%, such as more than 95% or even more than 99%, with SEQ ID NO: 1453, wherein the CDRs are as defined above. In one embodiment, the ISVD specifically binding to PD-1 comprises or consists of the amino acid sequence of SEQ ID NO: 1453.

In another embodiment, the ISVD specifically binding to human PD-1 may have a sequence identity of more than 90%, such as more than 95% or even more than 99%, with SEQ ID NO: 1454, wherein the CDRs are as defined above. In one embodiment, the ISVD specifically binding to PD-1 comprises or consists of the amino acid sequence of SEQ ID NO: 1454.

In another embodiment, the ISVD specifically binding to human PD-1 may have a sequence identity of more than 90%, such as more than 95% or even more than 99%, with SEQ ID NO: 1455, wherein the CDRs are as defined above. In one embodiment, the ISVD specifically binding to PD-1 comprises or consists of the amino acid sequence of SEQ ID NO: 1455.

In another embodiment, the ISVD specifically binding to human PD-1 may have a sequence identity of more than 90%, such as more than 95% or even more than 99%, with SEQ ID NO: 1456, wherein the CDRs are as defined above. In one embodiment, the ISVD specifically binding to PD-1 comprises or consists of the amino acid sequence of SEQ ID NO: 1456.

In another embodiment, the ISVD specifically binding to human PD-1 may have a sequence identity of more than 90%, such as more than 95% or even more than 99%, with SEQ ID NO: 1457, wherein the CDRs are as defined above. In one embodiment, the ISVD specifically binding to PD-1 comprises or consists of the amino acid sequence of SEQ ID NO: 1457.

In another embodiment, the ISVD specifically binding to human PD-1 may have a sequence identity of more than 90%, such as more than 95% or even more than 99%, with SEQ ID NO: 1458, wherein the CDRs are as defined above. In one embodiment, the ISVD specifically binding to PD-1 comprises or consists of the amino acid sequence of SEQ ID NO: 1458.

In another embodiment, the ISVD specifically binding to human PD-1 may have a sequence identity of more than 90%, such as more than 95% or even more than 99%, with SEQ ID NO: 1459, wherein the CDRs are as defined above. In one embodiment, the ISVD specifically binding to PD-1 comprises or consists of the amino acid sequence of SEQ ID NO: 1459.

In another embodiment, the ISVD specifically binding to human PD-1 may have a sequence identity of more than 90%, such as more than 95% or even more than 99%, with SEQ ID NO: 1460, wherein the CDRs are as defined above. In one embodiment, the ISVD specifically binding to PD-1 comprises or consists of the amino acid sequence of SEQ ID NO: 1460.

In another embodiment, the ISVD specifically binding to human PD-1 may have a sequence identity of more than 90%, such as more than 95% or even more than 99%, with SEQ ID NO: 1461, wherein the CDRs are as defined above. In one embodiment, the ISVD specifically binding to PD-1 comprises or consists of the amino acid sequence of SEQ ID NO: 1461.

In another embodiment, the ISVD specifically binding to human PD-1 may have a sequence identity of more than 90%, such as more than 95% or even more than 99%, with SEQ ID NO: 1462, wherein the CDRs are as defined above. In one embodiment, the ISVD specifically binding to PD-1 comprises or consists of the amino acid sequence of SEQ ID NO: 1462.

In another embodiment, the ISVD specifically binding to human PD-1 may have a sequence identity of more than 90%, such as more than 95% or even more than 99%, with SEQ ID NO: 1463, wherein the CDRs are as defined above. In one embodiment, the ISVD specifically binding to PD-1 comprises or consists of the amino acid sequence of SEQ ID NO: 1463.

In another embodiment, the ISVD specifically binding to human PD-1 may have a sequence identity of more than 90%, such as more than 95% or even more than 99%, with SEQ ID NO: 1464, wherein the CDRs are as defined above. In one embodiment, the ISVD specifically binding to PD-1 comprises or consists of the amino acid sequence of SEQ ID NO: 1464.

In another embodiment, the ISVD specifically binding to human PD-1 may have a sequence identity of more than 90%, such as more than 95% or even more than 99%, with SEQ ID NO: 1465, wherein the CDRs are as defined above. In one embodiment, the ISVD specifically binding to PD-1 comprises or consists of the amino acid sequence of SEQ ID NO: 1465.

In another embodiment, the ISVD specifically binding to human PD-1 may have a sequence identity of more than 90%, such as more than 95% or even more than 99%, with SEQ ID NO: 1466, wherein the CDRs are as defined above. In one embodiment, the ISVD specifically binding to PD-1 comprises or consists of the amino acid sequence of SEQ ID NO: 1466.

In another embodiment, the ISVD specifically binding to human PD-1 may have a sequence identity of more than 90%, such as more than 95% or even more than 99%, with SEQ ID NO: 1467, wherein the CDRs are as defined above. In one embodiment, the ISVD specifically binding to PD-1 comprises or consists of the amino acid sequence of SEQ ID NO: 1467.

In another embodiment, the ISVD specifically binding to human PD-1 may have a sequence identity of more than 90%, such as more than 95% or even more than 99%, with SEQ ID NO: 1468, wherein the CDRs are as defined above. In one embodiment, the ISVD specifically binding to PD-1 comprises or consists of the amino acid sequence of SEQ ID NO: 1468.

In another embodiment, the ISVD specifically binding to human PD-1 may have a sequence identity of more than 90%, such as more than 95% or even more than 99%, with SEQ ID NO: 1469, wherein the CDRs are as defined above. In one embodiment, the ISVD specifically binding to PD-1 comprises or consists of the amino acid sequence of SEQ ID NO: 1469.

In another embodiment, the ISVD specifically binding to human PD-1 may have a sequence identity of more than 90%, such as more than 95% or even more than 99%, with SEQ ID NO: 1470, wherein the CDRs are as defined above. In one embodiment, the ISVD specifically binding to PD-1 comprises or consists of the amino acid sequence of SEQ ID NO: 1470.

In another embodiment, the ISVD specifically binding to human PD-1 may have a sequence identity of more than 90%, such as more than 95% or even more than 99%, with SEQ ID NO: 1471, wherein the CDRs are as defined above. In one embodiment, the ISVD specifically binding to PD-1 comprises or consists of the amino acid sequence of SEQ ID NO: 1471.

In another embodiment, the ISVD specifically binding to human PD-1 may have a sequence identity of more than 90%, such as more than 95% or even more than 99%, with SEQ ID NO: 1472, wherein the CDRs are as defined above. In one embodiment, the ISVD specifically binding to PD-1 comprises or consists of the amino acid sequence of SEQ ID NO: 1472.

In another embodiment, the ISVD specifically binding to human PD-1 may have a sequence identity of more than 90%, such as more than 95% or even more than 99%, with SEQ ID NO: 1473, wherein the CDRs are as defined above. In one embodiment, the ISVD specifically binding to PD-1 comprises or consists of the amino acid sequence of SEQ ID NO: 1473.

In another embodiment, the ISVD specifically binding to human PD-1 may have a sequence identity of more than 90%, such as more than 95% or even more than 99%, with SEQ ID NO: 1474, wherein the CDRs are as defined above. In one embodiment, the ISVD specifically binding to PD-1 comprises or consists of the amino acid sequence of SEQ ID NO: 1474.

In another embodiment, the ISVD specifically binding to human PD-1 may have a sequence identity of more than 90%, such as more than 95% or even more than 99%, with SEQ ID NO: 1475, wherein the CDRs are as defined above. In one embodiment, the ISVD specifically binding to PD-1 comprises or consists of the amino acid sequence of SEQ ID NO: 1475.

In another embodiment, the ISVD specifically binding to human PD-1 may have a sequence identity of more than 90%, such as more than 95% or even more than 99%, with SEQ ID NO: 1476, wherein the CDRs are as defined above. In one embodiment, the ISVD specifically binding to PD-1 comprises or consists of the amino acid sequence of SEQ ID NO: 1476.

In another embodiment, the ISVD specifically binding to human PD-1 may have a sequence identity of more than 90%, such as more than 95% or even more than 99%, with SEQ ID NO: 1477, wherein the CDRs are as defined above. In one embodiment, the ISVD specifically binding to PD-1 comprises or consists of the amino acid sequence of SEQ ID NO: 1477.

In another embodiment, the ISVD specifically binding to human PD-1 may have a sequence identity of more than 90%, such as more than 95% or even more than 99%, with SEQ ID NO: 1478, wherein the CDRs are as defined above. In one embodiment, the ISVD specifically binding to PD-1 comprises or consists of the amino acid sequence of SEQ ID NO: 1478.

In another embodiment, the ISVD specifically binding to human PD-1 may have a sequence identity of more than 90%, such as more than 95% or even more than 99%, with SEQ ID NO: 1479, wherein the CDRs are as defined above. In one embodiment, the ISVD specifically binding to PD-1 comprises or consists of the amino acid sequence of SEQ ID NO: 1479.

In another embodiment, the ISVD specifically binding to human PD-1 may have a sequence identity of more than 90%, such as more than 95% or even more than 99%, with SEQ ID NO: 1480, wherein the CDRs are as defined above. In one embodiment, the ISVD specifically binding to PD-1 comprises or consists of the amino acid sequence of SEQ ID NO: 1480.

In another embodiment, the ISVD specifically binding to human PD-1 may have a sequence identity of more than 90%, such as more than 95% or even more than 99%, with SEQ ID NO: 1481, wherein the CDRs are as defined above. In one embodiment, the ISVD specifically binding to PD-1 comprises or consists of the amino acid sequence of SEQ ID NO: 1481.

In another embodiment, the ISVD specifically binding to human PD-1 may have a sequence identity of more than 90%, such as more than 95% or even more than 99%, with SEQ ID NO: 1482, wherein the CDRs are as defined above. In one embodiment, the ISVD specifically binding to PD-1 comprises or consists of the amino acid sequence of SEQ ID NO: 1482.

In another embodiment, the ISVD specifically binding to human PD-1 may have a sequence identity of more than 90%, such as more than 95% or even more than 99%, with SEQ ID NO: 1483, wherein the CDRs are as defined above. In one embodiment, the ISVD specifically binding to PD-1 comprises or consists of the amino acid sequence of SEQ ID NO: 1483.

In another embodiment, the ISVD specifically binding to human PD-1 may have a sequence identity of more than 90%, such as more than 95% or even more than 99%, with SEQ ID NO: 1484, wherein the CDRs are as defined above. In one embodiment, the ISVD specifically binding to PD-1 comprises or consists of the amino acid sequence of SEQ ID NO: 1484.

In another embodiment, the ISVD specifically binding to human PD-1 may have a sequence identity of more than 90%, such as more than 95% or even more than 99%, with SEQ ID NO: 1485, wherein the CDRs are as defined above. In one embodiment, the ISVD specifically binding to PD-1 comprises or consists of the amino acid sequence of SEQ ID NO: 1485.

In another embodiment, the ISVD specifically binding to human PD-1 may have a sequence identity of more than 90%, such as more than 95% or even more than 99%, with SEQ ID NO: 1486, wherein the CDRs are as defined above. In one embodiment, the ISVD specifically binding to PD-1 comprises or consists of the amino acid sequence of SEQ ID NO: 1486.

In another embodiment, the ISVD specifically binding to human PD-1 may have a sequence identity of more than 90%, such as more than 95% or even more than 99%, with SEQ ID NO: 1487, wherein the CDRs are as defined above. In one embodiment, the ISVD specifically binding to PD-1 comprises or consists of the amino acid sequence of SEQ ID NO: 1487.

In another embodiment, the ISVD specifically binding to human PD-1 may have a sequence identity of more than 90%, such as more than 95% or even more than 99%, with SEQ ID NO: 1488, wherein the CDRs are as defined above. In one embodiment, the ISVD specifically binding to PD-1 comprises or consists of the amino acid sequence of SEQ ID NO: 1488.

In another embodiment, the ISVD specifically binding to human PD-1 may have a sequence identity of more than 90%, such as more than 95% or even more than 99%, with SEQ ID NO: 1489, wherein the CDRs are as defined above. In one embodiment, the ISVD specifically binding to PD-1 comprises or consists of the amino acid sequence of SEQ ID NO: 1489.

In another embodiment, the ISVD specifically binding to human PD-1 may have a sequence identity of more than 90%, such as more than 95% or even more than 99%, with SEQ ID NO: 1490, wherein the CDRs are as defined above. In one embodiment, the ISVD specifically binding to PD-1 comprises or consists of the amino acid sequence of SEQ ID NO: 1490.

In another embodiment, the ISVD specifically binding to human PD-1 may have a sequence identity of more than 90%, such as more than 95% or even more than 99%, with SEQ ID NO: 1491, wherein the CDRs are as defined above. In one embodiment, the ISVD specifically binding to PD-1 comprises or consists of the amino acid sequence of SEQ ID NO: 1491.

In another embodiment, the ISVD specifically binding to human PD-1 may have a sequence identity of more than 90%, such as more than 95% or even more than 99%, with SEQ ID NO: 1492, wherein the CDRs are as defined above. In one embodiment, the ISVD specifically binding to PD-1 comprises or consists of the amino acid sequence of SEQ ID NO: 1492.

In another embodiment, the ISVD specifically binding to human PD-1 may have a sequence identity of more than 90%, such as more than 95% or even more than 99%, with SEQ ID NO: 1493, wherein the CDRs are as defined above. In one embodiment, the ISVD specifically binding to PD-1 comprises or consists of the amino acid sequence of SEQ ID NO: 1493.

In another embodiment, the ISVD specifically binding to human PD-1 may have a sequence identity of more than 90%, such as more than 95% or even more than 99%, with SEQ ID NO: 1494, wherein the CDRs are as defined above. In one embodiment, the ISVD specifically binding to PD-1 comprises or consists of the amino acid sequence of SEQ ID NO: 1494.

In another embodiment, the ISVD specifically binding to human PD-1 may have a sequence identity of more than 90%, such as more than 95% or even more than 99%, with SEQ ID NO: 1495, wherein the CDRs are as defined above. In one embodiment, the ISVD specifically binding to PD-1 comprises or consists of the amino acid sequence of SEQ ID NO: 1495.

In another embodiment, the ISVD specifically binding to human PD-1 may have a sequence identity of more than 90%, such as more than 95% or even more than 99%, with SEQ ID NO: 1496, wherein the CDRs are as defined above. In one embodiment, the ISVD specifically binding to PD-1 comprises or consists of the amino acid sequence of SEQ ID NO: 1496.

In another embodiment, the ISVD specifically binding to human PD-1 may have a sequence identity of more than 90%, such as more than 95% or even more than 99%, with SEQ ID NO: 1497, wherein the CDRs are as defined above. In one embodiment, the ISVD specifically binding to PD-1 comprises or consists of the amino acid sequence of SEQ ID NO: 1497.

In another embodiment, the ISVD specifically binding to human PD-1 may have a sequence identity of more than 90%, such as more than 95% or even more than 99%, with SEQ ID NO: 1498, wherein the CDRs are as defined above. In one embodiment, the ISVD specifically binding to PD-1 comprises or consists of the amino acid sequence of SEQ ID NO: 1498.

In another embodiment, the ISVD specifically binding to human PD-1 may have a sequence identity of more than 90%, such as more than 95% or even more than 99%, with SEQ ID NO: 1499, wherein the CDRs are as defined above. In one embodiment, the ISVD specifically binding to PD-1 comprises or consists of the amino acid sequence of SEQ ID NO: 1499.

In another embodiment, the ISVD specifically binding to human PD-1 may have a sequence identity of more than 90%, such as more than 95% or even more than 99%, with SEQ ID NO: 1500, wherein the CDRs are as defined above. In one embodiment, the ISVD specifically binding to PD-1 comprises or consists of the amino acid sequence of SEQ ID NO: 1500.

In another embodiment, the ISVD specifically binding to human PD-1 may have a sequence identity of more than 90%, such as more than 95% or even more than 99%, with SEQ ID NO: 1501, wherein the CDRs are as defined above. In one embodiment, the ISVD specifically binding to PD-1 comprises or consists of the amino acid sequence of SEQ ID NO: 1501.

In another embodiment, the ISVD specifically binding to human PD-1 may have a sequence identity of more than 90%, such as more than 95% or even more than 99%, with SEQ ID NO: 1502, wherein the CDRs are as defined above. In one embodiment, the ISVD specifically binding to PD-1 comprises or consists of the amino acid sequence of SEQ ID NO: 1502.

In another embodiment, the ISVD specifically binding to human PD-1 may have a sequence identity of more than 90%, such as more than 95% or even more than 99%, with SEQ ID NO: 1503, wherein the CDRs are as defined above. In one embodiment, the ISVD specifically binding to PD-1 comprises or consists of the amino acid sequence of SEQ ID NO: 1503.

In another embodiment, the ISVD specifically binding to human PD-1 may have a sequence identity of more than 90%, such as more than 95% or even more than 99%, with SEQ ID NO: 1504, wherein the CDRs are as defined above. In one embodiment, the ISVD specifically binding to PD-1 comprises or consists of the amino acid sequence of SEQ ID NO: 1504.

In another embodiment, the ISVD specifically binding to human PD-1 may have a sequence identity of more than 90%, such as more than 95% or even more than 99%, with SEQ ID NO: 1505, wherein the CDRs are as defined above. In one embodiment, the ISVD specifically binding to PD-1 comprises or consists of the amino acid sequence of SEQ ID NO: 1505.

In another embodiment, the ISVD specifically binding to human PD-1 may have a sequence identity of more than 90%, such as more than 95% or even more than 99%, with SEQ ID NO: 1506, wherein the CDRs are as defined above. In one embodiment, the ISVD specifically binding to PD-1 comprises or consists of the amino acid sequence of SEQ ID NO: 1506.

In another embodiment, the ISVD specifically binding to human PD-1 may have a sequence identity of more than 90%, such as more than 95% or even more than 99%, with SEQ ID NO: 1507, wherein the CDRs are as defined above. In one embodiment, the ISVD specifically binding to PD-1 comprises or consists of the amino acid sequence of SEQ ID NO: 1507.

In another embodiment, the ISVD specifically binding to human PD-1 may have a sequence identity of more than 90%, such as more than 95% or even more than 99%, with SEQ ID NO: 1508, wherein the CDRs are as defined above. In one embodiment, the ISVD specifically binding to PD-1 comprises or consists of the amino acid sequence of SEQ ID NO: 1508.

In another embodiment, the ISVD specifically binding to human PD-1 may have a sequence identity of more than 90%, such as more than 95% or even more than 99%, with SEQ ID NO: 1509, wherein the CDRs are as defined above. In one embodiment, the ISVD specifically binding to PD-1 comprises or consists of the amino acid sequence of SEQ ID NO: 1509.

In another embodiment, the ISVD specifically binding to human PD-1 may have a sequence identity of more than 90%, such as more than 95% or even more than 99%, with SEQ ID NO: 1510, wherein the CDRs are as defined above. In one embodiment, the ISVD specifically binding to PD-1 comprises or consists of the amino acid sequence of SEQ ID NO: 1510.

In another embodiment, the ISVD specifically binding to human PD-1 may have a sequence identity of more than 90%, such as more than 95% or even more than 99%, with SEQ ID NO: 1511, wherein the CDRs are as defined above. In one embodiment, the ISVD specifically binding to PD-1 comprises or consists of the amino acid sequence of SEQ ID NO: 1511.

In another embodiment, the ISVD specifically binding to human PD-1 may have a sequence identity of more than 90%, such as more than 95% or even more than 99%, with SEQ ID NO: 1512, wherein the CDRs are as defined above. In one embodiment, the ISVD specifically binding to PD-1 comprises or consists of the amino acid sequence of SEQ ID NO: 1512.

In another embodiment, the ISVD specifically binding to human PD-1 may have a sequence identity of more than 90%, such as more than 95% or even more than 99%, with SEQ ID NO: 1513, wherein the CDRs are as defined above. In one embodiment, the ISVD specifically binding to PD-1 comprises or consists of the amino acid sequence of SEQ ID NO: 1513.

In another embodiment, the ISVD specifically binding to human PD-1 may have a sequence identity of more than 90%, such as more than 95% or even more than 99%, with SEQ ID NO: 1514, wherein the CDRs are as defined above. In one embodiment, the ISVD specifically binding to PD-1 comprises or consists of the amino acid sequence of SEQ ID NO: 1514.

In another embodiment, the ISVD specifically binding to human PD-1 may have a sequence identity of more than 90%, such as more than 95% or even more than 99%, with SEQ ID NO: 1515, wherein the CDRs are as defined above. In one embodiment, the ISVD specifically binding to PD-1 comprises or consists of the amino acid sequence of SEQ ID NO: 1515.

In another embodiment, the ISVD specifically binding to human PD-1 may have a sequence identity of more than 90%, such as more than 95% or even more than 99%, with SEQ ID NO: 1516, wherein the CDRs are as defined above. In one embodiment, the ISVD specifically binding to PD-1 comprises or consists of the amino acid sequence of SEQ ID NO: 1516.

In another embodiment, the ISVD specifically binding to human PD-1 may have a sequence identity of more than 90%, such as more than 95% or even more than 99%, with SEQ ID NO: 1517, wherein the CDRs are as defined above. In one embodiment, the ISVD specifically binding to PD-1 comprises or consists of the amino acid sequence of SEQ ID NO: 1517.

In another embodiment, the ISVD specifically binding to human PD-1 may have a sequence identity of more than 90%, such as more than 95% or even more than 99%, with SEQ ID NO: 1518, wherein the CDRs are as defined above. In one embodiment, the ISVD specifically binding to PD-1 comprises or consists of the amino acid sequence of SEQ ID NO: 1518.

In another embodiment, the ISVD specifically binding to human PD-1 may have a sequence identity of more than 90%, such as more than 95% or even more than 99%, with SEQ ID NO: 1519, wherein the CDRs are as defined above. In one embodiment, the ISVD specifically binding to PD-1 comprises or consists of the amino acid sequence of SEQ ID NO: 1519.

In another embodiment, the ISVD specifically binding to human PD-1 may have a sequence identity of more than 90%, such as more than 95% or even more than 99%, with SEQ ID NO: 1520, wherein the CDRs are as defined above. In one embodiment, the ISVD specifically binding to PD-1 comprises or consists of the amino acid sequence of SEQ ID NO: 1520.

In another embodiment, the ISVD specifically binding to human PD-1 may have a sequence identity of more than 90%, such as more than 95% or even more than 99%, with SEQ ID NO: 1521, wherein the CDRs are as defined above. In one embodiment, the ISVD specifically binding to PD-1 comprises or consists of the amino acid sequence of SEQ ID NO: 1521.

In another embodiment, the ISVD specifically binding to human PD-1 may have a sequence identity of more than 90%, such as more than 95% or even more than 99%, with SEQ ID NO: 1522, wherein the CDRs are as defined above. In one embodiment, the ISVD specifically binding to PD-1 comprises or consists of the amino acid sequence of SEQ ID NO: 1522.

In another embodiment, the ISVD specifically binding to human PD-1 may have a sequence identity of more than 90%, such as more than 95% or even more than 99%, with SEQ ID NO: 1523, wherein the CDRs are as defined above. In one embodiment, the ISVD specifically binding to PD-1 comprises or consists of the amino acid sequence of SEQ ID NO: 1523.

In another embodiment, the ISVD specifically binding to human PD-1 may have a sequence identity of more than 90%, such as more than 95% or even more than 99%, with SEQ ID NO: 1524, wherein the CDRs are as defined above. In one embodiment, the ISVD specifically binding to PD-1 comprises or consists of the amino acid sequence of SEQ ID NO: 1524.

In another embodiment, the ISVD specifically binding to human PD-1 may have a sequence identity of more than 90%, such as more than 95% or even more than 99%, with SEQ ID NO: 1525, wherein the CDRs are as defined above. In one embodiment, the ISVD specifically binding to PD-1 comprises or consists of the amino acid sequence of SEQ ID NO: 1525.

In another embodiment, the ISVD specifically binding to human PD-1 may have a sequence identity of more than 90%, such as more than 95% or even more than 99%, with SEQ ID NO: 1526, wherein the CDRs are as defined above. In one embodiment, the ISVD specifically binding to PD-1 comprises or consists of the amino acid sequence of SEQ ID NO: 1526.

In another embodiment, the ISVD specifically binding to human PD-1 may have a sequence identity of more than 90%, such as more than 95% or even more than 99%, with SEQ ID NO: 1527, wherein the CDRs are as defined above. In one embodiment, the ISVD specifically binding to PD-1 comprises or consists of the amino acid sequence of SEQ ID NO: 1527.

In another embodiment, the ISVD specifically binding to human PD-1 may have a sequence identity of more than 90%, such as more than 95% or even more than 99%, with SEQ ID NO: 1528, wherein the CDRs are as defined above. In one embodiment, the ISVD specifically binding to PD-1 comprises or consists of the amino acid sequence of SEQ ID NO: 1528.

In another embodiment, the ISVD specifically binding to human PD-1 may have a sequence identity of more than 90%, such as more than 95% or even more than 99%, with SEQ ID NO: 1529, wherein the CDRs are as defined above. In one embodiment, the ISVD specifically binding to PD-1 comprises or consists of the amino acid sequence of SEQ ID NO: 1529.

In another embodiment, the ISVD specifically binding to human PD-1 may have a sequence identity of more than 90%, such as more than 95% or even more than 99%, with SEQ ID NO: 1530, wherein the CDRs are as defined above. In one embodiment, the ISVD specifically binding to PD-1 comprises or consists of the amino acid sequence of SEQ ID NO: 1530.

In another embodiment, the ISVD specifically binding to human PD-1 may have a sequence identity of more than 90%, such as more than 95% or even more than 99%, with SEQ ID NO: 1531, wherein the CDRs are as defined above. In one embodiment, the ISVD specifically binding to PD-1 comprises or consists of the amino acid sequence of SEQ ID NO: 1531.

In another embodiment, the ISVD specifically binding to human PD-1 may have a sequence identity of more than 90%, such as more than 95% or even more than 99%, with SEQ ID NO: 1532, wherein the CDRs are as defined above. In one embodiment, the ISVD specifically binding to PD-1 comprises or consists of the amino acid sequence of SEQ ID NO: 1532.

In another embodiment, the ISVD specifically binding to human PD-1 may have a sequence identity of more than 90%, such as more than 95% or even more than 99%, with SEQ ID NO: 1533, wherein the CDRs are as defined above. In one embodiment, the ISVD specifically binding to PD-1 comprises or consists of the amino acid sequence of SEQ ID NO: 1533.

In another embodiment, the ISVD specifically binding to human PD-1 may have a sequence identity of more than 90%, such as more than 95% or even more than 99%, with SEQ ID NO: 1534, wherein the CDRs are as defined above. In one embodiment, the ISVD specifically binding to PD-1 comprises or consists of the amino acid sequence of SEQ ID NO: 1534.

In another embodiment, the ISVD specifically binding to human PD-1 may have a sequence identity of more than 90%, such as more than 95% or even more than 99%, with SEQ ID NO: 1535, wherein the CDRs are as defined above. In one embodiment, the ISVD specifically binding to PD-1 comprises or consists of the amino acid sequence of SEQ ID NO: 1535.

In another embodiment, the ISVD specifically binding to human PD-1 may have a sequence identity of more than 90%, such as more than 95% or even more than 99%, with SEQ ID NO: 1536, wherein the CDRs are as defined above. In one embodiment, the ISVD specifically binding to PD-1 comprises or consists of the amino acid sequence of SEQ ID NO: 1536.

In another embodiment, the ISVD specifically binding to human PD-1 may have a sequence identity of more than 90%, such as more than 95% or even more than 99%, with SEQ ID NO: 1537, wherein the CDRs are as defined above. In one embodiment, the ISVD specifically binding to PD-1 comprises or consists of the amino acid sequence of SEQ ID NO: 1537.

In another embodiment, the ISVD specifically binding to human PD-1 may have a sequence identity of more than 90%, such as more than 95% or even more than 99%, with SEQ ID NO: 1538, wherein the CDRs are as defined above. In one embodiment, the ISVD specifically binding to PD-1 comprises or consists of the amino acid sequence of SEQ ID NO: 1538.

In another embodiment, the ISVD specifically binding to human PD-1 may have a sequence identity of more than 90%, such as more than 95% or even more than 99%, with SEQ ID NO: 1539, wherein the CDRs are as defined above. In one embodiment, the ISVD specifically binding to PD-1 comprises or consists of the amino acid sequence of SEQ ID NO: 1539.

In another embodiment, the ISVD specifically binding to human PD-1 may have a sequence identity of more than 90%, such as more than 95% or even more than 99%, with SEQ ID NO: 1540, wherein the CDRs are as defined above. In one embodiment, the ISVD specifically binding to PD-1 comprises or consists of the amino acid sequence of SEQ ID NO: 1540.

In another embodiment, the ISVD specifically binding to human PD-1 may have a sequence identity of more than 90%, such as more than 95% or even more than 99%, with SEQ ID NO: 1541, wherein the CDRs are as defined above. In one embodiment, the ISVD specifically binding to PD-1 comprises or consists of the amino acid sequence of SEQ ID NO: 1541.

In another embodiment, the ISVD specifically binding to human PD-1 may have a sequence identity of more than 90%, such as more than 95% or even more than 99%, with SEQ ID NO: 1542, wherein the CDRs are as defined above. In one embodiment, the ISVD specifically binding to PD-1 comprises or consists of the amino acid sequence of SEQ ID NO: 1542.

In another embodiment, the ISVD specifically binding to human PD-1 may have a sequence identity of more than 90%, such as more than 95% or even more than 99%, with SEQ ID NO: 1543, wherein the CDRs are as defined above. In one embodiment, the ISVD specifically binding to PD-1 comprises or consists of the amino acid sequence of SEQ ID NO: 1543.

In another embodiment, the ISVD specifically binding to human PD-1 may have a sequence identity of more than 90%, such as more than 95% or even more than 99%, with SEQ ID NO: 1544, wherein the CDRs are as defined above. In one embodiment, the ISVD specifically binding to PD-1 comprises or consists of the amino acid sequence of SEQ ID NO: 1544.

In another embodiment, the ISVD specifically binding to human PD-1 may have a sequence identity of more than 90%, such as more than 95% or even more than 99%, with SEQ ID NO: 1545, wherein the CDRs are as defined above. In one embodiment, the ISVD specifically binding to PD-1 comprises or consists of the amino acid sequence of SEQ ID NO: 1545.

In another embodiment, the ISVD specifically binding to human PD-1 may have a sequence identity of more than 90%, such as more than 95% or even more than 99%, with SEQ ID NO: 1546, wherein the CDRs are as defined above. In one embodiment, the ISVD specifically binding to PD-1 comprises or consists of the amino acid sequence of SEQ ID NO: 1546.

In another embodiment, the ISVD specifically binding to human PD-1 may have a sequence identity of more than 90%, such as more than 95% or even more than 99%, with SEQ ID NO: 1547, wherein the CDRs are as defined above. In one embodiment, the ISVD specifically binding to PD-1 comprises or consists of the amino acid sequence of SEQ ID NO: 1547.

In another embodiment, the ISVD specifically binding to human PD-1 may have a sequence identity of more than 90%, such as more than 95% or even more than 99%, with SEQ ID NO: 1548, wherein the CDRs are as defined above. In one embodiment, the ISVD specifically binding to PD-1 comprises or consists of the amino acid sequence of SEQ ID NO: 1548.

In another embodiment, the ISVD specifically binding to human PD-1 may have a sequence identity of more than 90%, such as more than 95% or even more than 99%, with SEQ ID NO: 1549, wherein the CDRs are as defined above. In one embodiment, the ISVD specifically binding to PD-1 comprises or consists of the amino acid sequence of SEQ ID NO: 1549.

In another embodiment, the ISVD specifically binding to human PD-1 may have a sequence identity of more than 90%, such as more than 95% or even more than 99%, with SEQ ID NO: 1550, wherein the CDRs are as defined above. In one embodiment, the ISVD specifically binding to PD-1 comprises or consists of the amino acid sequence of SEQ ID NO: 1550.

In another embodiment, the ISVD specifically binding to human PD-1 may have a sequence identity of more than 90%, such as more than 95% or even more than 99%, with SEQ ID NO: 1551, wherein the CDRs are as defined above. In one embodiment, the ISVD specifically binding to PD-1 comprises or consists of the amino acid sequence of SEQ ID NO: 1551.

In another embodiment, the ISVD specifically binding to human PD-1 may have a sequence identity of more than 90%, such as more than 95% or even more than 99%, with SEQ ID NO: 1552, wherein the CDRs are as defined above. In one embodiment, the ISVD specifically binding to PD-1 comprises or consists of the amino acid sequence of SEQ ID NO: 1552.

In another embodiment, the ISVD specifically binding to human PD-1 may have a sequence identity of more than 90%, such as more than 95% or even more than 99%, with SEQ ID NO: 1553, wherein the CDRs are as defined above. In one embodiment, the ISVD specifically binding to PD-1 comprises or consists of the amino acid sequence of SEQ ID NO: 1553.

In another embodiment, the ISVD specifically binding to human PD-1 may have a sequence identity of more than 90%, such as more than 95% or even more than 99%, with SEQ ID NO: 1554, wherein the CDRs are as defined above. In one embodiment, the ISVD specifically binding to PD-1 comprises or consists of the amino acid sequence of SEQ ID NO: 1554.

In another embodiment, the ISVD specifically binding to human PD-1 may have a sequence identity of more than 90%, such as more than 95% or even more than 99%, with SEQ ID NO: 1555, wherein the CDRs are as defined above. In one embodiment, the ISVD specifically binding to PD-1 comprises or consists of the amino acid sequence of SEQ ID NO: 1555.

In another embodiment, the ISVD specifically binding to human PD-1 may have a sequence identity of more than 90%, such as more than 95% or even more than 99%, with SEQ ID NO: 1556, wherein the CDRs are as defined above. In one embodiment, the ISVD specifically binding to PD-1 comprises or consists of the amino acid sequence of SEQ ID NO: 1556.

In another embodiment, the ISVD specifically binding to human PD-1 may have a sequence identity of more than 90%, such as more than 95% or even more than 99%, with SEQ ID NO: 1557, wherein the CDRs are as defined above. In one embodiment, the ISVD specifically binding to PD-1 comprises or consists of the amino acid sequence of SEQ ID NO: 1557.

In another embodiment, the ISVD specifically binding to human PD-1 may have a sequence identity of more than 90%, such as more than 95% or even more than 99%, with SEQ ID NO: 1558, wherein the CDRs are as defined above. In one embodiment, the ISVD specifically binding to PD-1 comprises or consists of the amino acid sequence of SEQ ID NO: 1558.

In another embodiment, the ISVD specifically binding to human PD-1 may have a sequence identity of more than 90%, such as more than 95% or even more than 99%, with SEQ ID NO: 1559, wherein the CDRs are as defined above. In one embodiment, the ISVD specifically binding to PD-1 comprises or consists of the amino acid sequence of SEQ ID NO: 1559.

In another embodiment, the ISVD specifically binding to human PD-1 may have a sequence identity of more than 90%, such as more than 95% or even more than 99%, with SEQ ID NO: 1560, wherein the CDRs are as defined above. In one embodiment, the ISVD specifically binding to PD-1 comprises or consists of the amino acid sequence of SEQ ID NO: 1560.

In another embodiment, the ISVD specifically binding to human PD-1 may have a sequence identity of more than 90%, such as more than 95% or even more than 99%, with SEQ ID NO: 1561, wherein the CDRs are as defined above. In one embodiment, the ISVD specifically binding to PD-1 comprises or consists of the amino acid sequence of SEQ ID NO: 1561.

In another embodiment, the ISVD specifically binding to human PD-1 may have a sequence identity of more than 90%, such as more than 95% or even more than 99%, with SEQ ID NO: 1562, wherein the CDRs are as defined above. In one embodiment, the ISVD specifically binding to PD-1 comprises or consists of the amino acid sequence of SEQ ID NO: 1562.

In another embodiment, the ISVD specifically binding to human PD-1 may have a sequence identity of more than 90%, such as more than 95% or even more than 99%, with SEQ ID NO: 1563, wherein the CDRs are as defined above. In one embodiment, the ISVD specifically binding to PD-1 comprises or consists of the amino acid sequence of SEQ ID NO: 1563.

In another embodiment, the ISVD specifically binding to human PD-1 may have a sequence identity of more than 90%, such as more than 95% or even more than 99%, with SEQ ID NO: 1564, wherein the CDRs are as defined above. In one embodiment, the ISVD specifically binding to PD-1 comprises or consists of the amino acid sequence of SEQ ID NO: 1564.

In another embodiment, the ISVD specifically binding to human PD-1 may have a sequence identity of more than 90%, such as more than 95% or even more than 99%, with SEQ ID NO: 1565, wherein the CDRs are as defined above. In one embodiment, the ISVD specifically binding to PD-1 comprises or consists of the amino acid sequence of SEQ ID NO: 1565.

In another embodiment, the ISVD specifically binding to human PD-1 may have a sequence identity of more than 90%, such as more than 95% or even more than 99%, with SEQ ID NO: 1566, wherein the CDRs are as defined above. In one embodiment, the ISVD specifically binding to PD-1 comprises or consists of the amino acid sequence of SEQ ID NO: 1566.

In another embodiment, the ISVD specifically binding to human PD-1 may have a sequence identity of more than 90%, such as more than 95% or even more than 99%, with SEQ ID NO: 1567, wherein the CDRs are as defined above. In one embodiment, the ISVD specifically binding to PD-1 comprises or consists of the amino acid sequence of SEQ ID NO: 1567.

In another embodiment, the ISVD specifically binding to human PD-1 may have a sequence identity of more than 90%, such as more than 95% or even more than 99%, with SEQ ID NO: 1568, wherein the CDRs are as defined above. In one embodiment, the ISVD specifically binding to PD-1 comprises or consists of the amino acid sequence of SEQ ID NO: 1568.

In another embodiment, the ISVD specifically binding to human PD-1 may have a sequence identity of more than 90%, such as more than 95% or even more than 99%, with SEQ ID NO: 1569, wherein the CDRs are as defined above. In one embodiment, the ISVD specifically binding to PD-1 comprises or consists of the amino acid sequence of SEQ ID NO: 1569.

In another embodiment, the ISVD specifically binding to human PD-1 may have a sequence identity of more than 90%, such as more than 95% or even more than 99%, with SEQ ID NO: 1570, wherein the CDRs are as defined above. In one embodiment, the ISVD specifically binding to PD-1 comprises or consists of the amino acid sequence of SEQ ID NO: 1570.

In another embodiment, the ISVD specifically binding to human PD-1 may have a sequence identity of more than 90%, such as more than 95% or even more than 99%, with SEQ ID NO: 1571, wherein the CDRs are as defined above. In one embodiment, the ISVD specifically binding to PD-1 comprises or consists of the amino acid sequence of SEQ ID NO: 1571.

In another embodiment, the ISVD specifically binding to human PD-1 may have a sequence identity of more than 90%, such as more than 95% or even more than 99%, with SEQ ID NO: 1572, wherein the CDRs are as defined above. In one embodiment, the ISVD specifically binding to PD-1 comprises or consists of the amino acid sequence of SEQ ID NO: 1572.

In another embodiment, the ISVD specifically binding to human PD-1 may have a sequence identity of more than 90%, such as more than 95% or even more than 99%, with SEQ ID NO: 1573, wherein the CDRs are as defined above. In one embodiment, the ISVD specifically binding to PD-1 comprises or consists of the amino acid sequence of SEQ ID NO: 1573.

In another embodiment, the ISVD specifically binding to human PD-1 may have a sequence identity of more than 90%, such as more than 95% or even more than 99%, with SEQ ID NO: 1574, wherein the CDRs are as defined above. In one embodiment, the ISVD specifically binding to PD-1 comprises or consists of the amino acid sequence of SEQ ID NO: 1574.

In another embodiment, the ISVD specifically binding to human PD-1 may have a sequence identity of more than 90%, such as more than 95% or even more than 99%, with SEQ ID NO: 1575, wherein the CDRs are as defined above. In one embodiment, the ISVD specifically binding to PD-1 comprises or consists of the amino acid sequence of SEQ ID NO: 1575.

In another embodiment, the ISVD specifically binding to human PD-1 may have a sequence identity of more than 90%, such as more than 95% or even more than 99%, with SEQ ID NO: 1576, wherein the CDRs are as defined above. In one embodiment, the ISVD specifically binding to PD-1 comprises or consists of the amino acid sequence of SEQ ID NO: 1576.

In another embodiment, the ISVD specifically binding to human PD-1 may have a sequence identity of more than 90%, such as more than 95% or even more than 99%, with SEQ ID NO: 1577, wherein the CDRs are as defined above. In one embodiment, the ISVD specifically binding to PD-1 comprises or consists of the amino acid sequence of SEQ ID NO: 1577.

In another embodiment, the ISVD specifically binding to human PD-1 may have a sequence identity of more than 90%, such as more than 95% or even more than 99%, with SEQ ID NO: 1578, wherein the CDRs are as defined above. In one embodiment, the ISVD specifically binding to PD-1 comprises or consists of the amino acid sequence of SEQ ID NO: 1578.

In another embodiment, the ISVD specifically binding to human PD-1 may have a sequence identity of more than 90%, such as more than 95% or even more than 99%, with SEQ ID NO: 1579, wherein the CDRs are as defined above. In one embodiment, the ISVD specifically binding to PD-1 comprises or consists of the amino acid sequence of SEQ ID NO: 1579.

In another embodiment, the ISVD specifically binding to human PD-1 may have a sequence identity of more than 90%, such as more than 95% or even more than 99%, with SEQ ID NO: 1580, wherein the CDRs are as defined above. In one embodiment, the ISVD specifically binding to PD-1 comprises or consists of the amino acid sequence of SEQ ID NO: 1580.

In another embodiment, the ISVD specifically binding to human PD-1 may have a sequence identity of more than 90%, such as more than 95% or even more than 99%, with SEQ ID NO: 1581, wherein the CDRs are as defined above. In one embodiment, the ISVD specifically binding to PD-1 comprises or consists of the amino acid sequence of SEQ ID NO: 1581.

In another embodiment, the ISVD specifically binding to human PD-1 may have a sequence identity of more than 90%, such as more than 95% or even more than 99%, with SEQ ID NO: 1582, wherein the CDRs are as defined above. In one embodiment, the ISVD specifically binding to PD-1 comprises or consists of the amino acid sequence of SEQ ID NO: 1582.

In another embodiment, the ISVD specifically binding to human PD-1 may have a sequence identity of more than 90%, such as more than 95% or even more than 99%, with SEQ ID NO: 1583, wherein the CDRs are as defined above. In one embodiment, the ISVD specifically binding to PD-1 comprises or consists of the amino acid sequence of SEQ ID NO: 1583.

In another embodiment, the ISVD specifically binding to human PD-1 may have a sequence identity of more than 90%, such as more than 95% or even more than 99%, with SEQ ID NO: 1584, wherein the CDRs are as defined above. In one embodiment, the ISVD specifically binding to PD-1 comprises or consists of the amino acid sequence of SEQ ID NO: 1584.

In another embodiment, the ISVD specifically binding to human PD-1 may have a sequence identity of more than 90%, such as more than 95% or even more than 99%, with SEQ ID NO: 1585, wherein the CDRs are as defined above. In one embodiment, the ISVD specifically binding to PD-1 comprises or consists of the amino acid sequence of SEQ ID NO: 1585.

In another embodiment, the ISVD specifically binding to human PD-1 may have a sequence identity of more than 90%, such as more than 95% or even more than 99%, with SEQ ID NO: 1586, wherein the CDRs are as defined above. In one embodiment, the ISVD specifically binding to PD-1 comprises or consists of the amino acid sequence of SEQ ID NO: 1586.

In another embodiment, the ISVD specifically binding to human PD-1 may have a sequence identity of more than 90%, such as more than 95% or even more than 99%, with SEQ ID NO: 1587, wherein the CDRs are as defined above. In one embodiment, the ISVD specifically binding to PD-1 comprises or consists of the amino acid sequence of SEQ ID NO: 1587.

In another embodiment, the ISVD specifically binding to human PD-1 may have a sequence identity of more than 90%, such as more than 95% or even more than 99%, with SEQ ID NO: 1588, wherein the CDRs are as defined above. In one embodiment, the ISVD specifically binding to PD-1 comprises or consists of the amino acid sequence of SEQ ID NO: 1588.

In another embodiment, the ISVD specifically binding to human PD-1 may have a sequence identity of more than 90%, such as more than 95% or even more than 99%, with SEQ ID NO: 1589, wherein the CDRs are as defined above. In one embodiment, the ISVD specifically binding to PD-1 comprises or consists of the amino acid sequence of SEQ ID NO: 1589.

In another embodiment, the ISVD specifically binding to human PD-1 may have a sequence identity of more than 90%, such as more than 95% or even more than 99%, with SEQ ID NO: 1590, wherein the CDRs are as defined above. In one embodiment, the ISVD specifically binding to PD-1 comprises or consists of the amino acid sequence of SEQ ID NO: 1590.

In another embodiment, the ISVD specifically binding to human PD-1 may have a sequence identity of more than 90%, such as more than 95% or even more than 99%, with SEQ ID NO: 1591, wherein the CDRs are as defined above. In one embodiment, the ISVD specifically binding to PD-1 comprises or consists of the amino acid sequence of SEQ ID NO: 1591.

In another embodiment, the ISVD specifically binding to human PD-1 may have a sequence identity of more than 90%, such as more than 95% or even more than 99%, with SEQ ID NO: 1592, wherein the CDRs are as defined above. In one embodiment, the ISVD specifically binding to PD-1 comprises or consists of the amino acid sequence of SEQ ID NO: 1592.

In another embodiment, the ISVD specifically binding to human PD-1 may have a sequence identity of more than 90%, such as more than 95% or even more than 99%, with SEQ ID NO: 1593, wherein the CDRs are as defined above. In one embodiment, the ISVD specifically binding to PD-1 comprises or consists of the amino acid sequence of SEQ ID NO: 1593.

In another embodiment, the ISVD specifically binding to human PD-1 may have a sequence identity of more than 90%, such as more than 95% or even more than 99%, with SEQ ID NO: 1594, wherein the CDRs are as defined above. In one embodiment, the ISVD specifically binding to PD-1 comprises or consists of the amino acid sequence of SEQ ID NO: 1594.

In another embodiment, the ISVD specifically binding to human PD-1 may have a sequence identity of more than 90%, such as more than 95% or even more than 99%, with SEQ ID NO: 1595, wherein the CDRs are as defined above. In one embodiment, the ISVD specifically binding to PD-1 comprises or consists of the amino acid sequence of SEQ ID NO: 1595.

In another embodiment, the ISVD specifically binding to human PD-1 may have a sequence identity of more than 90%, such as more than 95% or even more than 99%, with SEQ ID NO: 1596, wherein the CDRs are as defined above. In one embodiment, the ISVD specifically binding to PD-1 comprises or consists of the amino acid sequence of SEQ ID NO: 1596.

In another embodiment, the ISVD specifically binding to human PD-1 may have a sequence identity of more than 90%, such as more than 95% or even more than 99%, with SEQ ID NO: 1597, wherein the CDRs are as defined above. In one embodiment, the ISVD specifically binding to PD-1 comprises or consists of the amino acid sequence of SEQ ID NO: 1597.

In another embodiment, the ISVD specifically binding to human PD-1 may have a sequence identity of more than 90%, such as more than 95% or even more than 99%, with SEQ ID NO: 1598, wherein the CDRs are as defined above. In one embodiment, the ISVD specifically binding to PD-1 comprises or consists of the amino acid sequence of SEQ ID NO: 1598.

In another embodiment, the ISVD specifically binding to human PD-1 may have a sequence identity of more than 90%, such as more than 95% or even more than 99%, with SEQ ID NO: 1599, wherein the CDRs are as defined above. In one embodiment, the ISVD specifically binding to PD-1 comprises or consists of the amino acid sequence of SEQ ID NO: 1599.

In another embodiment, the ISVD specifically binding to human PD-1 may have a sequence identity of more than 90%, such as more than 95% or even more than 99%, with SEQ ID NO: 1600, wherein the CDRs are as defined above. In one embodiment, the ISVD specifically binding to PD-1 comprises or consists of the amino acid sequence of SEQ ID NO: 1600.

In another embodiment, the ISVD specifically binding to human PD-1 may have a sequence identity of more than 90%, such as more than 95% or even more than 99%, with SEQ ID NO: 1601, wherein the CDRs are as defined above. In one embodiment, the ISVD specifically binding to PD-1 comprises or consists of the amino acid sequence of SEQ ID NO: 1601.

In another embodiment, the ISVD specifically binding to human PD-1 may have a sequence identity of more than 90%, such as more than 95% or even more than 99%, with SEQ ID NO: 1602, wherein the CDRs are as defined above. In one embodiment, the ISVD specifically binding to PD-1 comprises or consists of the amino acid sequence of SEQ ID NO: 1602.

In another embodiment, the ISVD specifically binding to human PD-1 may have a sequence identity of more than 90%, such as more than 95% or even more than 99%, with SEQ ID NO: 1603, wherein the CDRs are as defined above. In one embodiment, the ISVD specifically binding to PD-1 comprises or consists of the amino acid sequence of SEQ ID NO: 1603.

In another embodiment, the ISVD specifically binding to human PD-1 may have a sequence identity of more than 90%, such as more than 95% or even more than 99%, with SEQ ID NO: 1604, wherein the CDRs are as defined above. In one embodiment, the ISVD specifically binding to PD-1 comprises or consists of the amino acid sequence of SEQ ID NO: 1604.

In another embodiment, the ISVD specifically binding to human PD-1 may have a sequence identity of more than 90%, such as more than 95% or even more than 99%, with SEQ ID NO: 1605, wherein the CDRs are as defined above. In one embodiment, the ISVD specifically binding to PD-1 comprises or consists of the amino acid sequence of SEQ ID NO: 1605.

In another embodiment, the ISVD specifically binding to human PD-1 may have a sequence identity of more than 90%, such as more than 95% or even more than 99%, with SEQ ID NO: 1606, wherein the CDRs are as defined above. In one embodiment, the ISVD specifically binding to PD-1 comprises or consists of the amino acid sequence of SEQ ID NO: 1606.

In another embodiment, the ISVD specifically binding to human PD-1 may have a sequence identity of more than 90%, such as more than 95% or even more than 99%, with SEQ ID NO: 1607, wherein the CDRs are as defined above. In one embodiment, the ISVD specifically binding to PD-1 comprises or consists of the amino acid sequence of SEQ ID NO: 1607.

In another embodiment, the ISVD specifically binding to human PD-1 may have a sequence identity of more than 90%, such as more than 95% or even more than 99%, with SEQ ID NO: 1608, wherein the CDRs are as defined above. In one embodiment, the ISVD specifically binding to PD-1 comprises or consists of the amino acid sequence of SEQ ID NO: 1608.

In another embodiment, the ISVD specifically binding to human PD-1 may have a sequence identity of more than 90%, such as more than 95% or even more than 99%, with SEQ ID NO: 1609, wherein the CDRs are as defined above. In one embodiment, the ISVD specifically binding to PD-1 comprises or consists of the amino acid sequence of SEQ ID NO: 1609.

In another embodiment, the ISVD specifically binding to human PD-1 may have a sequence identity of more than 90%, such as more than 95% or even more than 99%, with SEQ ID NO: 1610, wherein the CDRs are as defined above. In one embodiment, the ISVD specifically binding to PD-1 comprises or consists of the amino acid sequence of SEQ ID NO: 1610.

In another embodiment, the ISVD specifically binding to human PD-1 may have a sequence identity of more than 90%, such as more than 95% or even more than 99%, with SEQ ID NO: 1611, wherein the CDRs are as defined above. In one embodiment, the ISVD specifically binding to PD-1 comprises or consists of the amino acid sequence of SEQ ID NO: 1611.

In another embodiment, the ISVD specifically binding to human PD-1 may have a sequence identity of more than 90%, such as more than 95% or even more than 99%, with SEQ ID NO: 1612, wherein the CDRs are as defined above. In one embodiment, the ISVD specifically binding to PD-1 comprises or consists of the amino acid sequence of SEQ ID NO: 1612.

In another embodiment, the ISVD specifically binding to human PD-1 may have a sequence identity of more than 90%, such as more than 95% or even more than 99%, with SEQ ID NO: 1613, wherein the CDRs are as defined above. In one embodiment, the ISVD specifically binding to PD-1 comprises or consists of the amino acid sequence of SEQ ID NO: 1613.

In another embodiment, the ISVD specifically binding to human PD-1 may have a sequence identity of more than 90%, such as more than 95% or even more than 99%, with SEQ ID NO: 1614, wherein the CDRs are as defined above. In one embodiment, the ISVD specifically binding to PD-1 comprises or consists of the amino acid sequence of SEQ ID NO: 1614.

In another embodiment, the ISVD specifically binding to human PD-1 may have a sequence identity of more than 90%, such as more than 95% or even more than 99%, with SEQ ID NO: 1615, wherein the CDRs are as defined above. In one embodiment, the ISVD specifically binding to PD-1 comprises or consists of the amino acid sequence of SEQ ID NO: 1615.

In another embodiment, the ISVD specifically binding to human PD-1 may have a sequence identity of more than 90%, such as more than 95% or even more than 99%, with SEQ ID NO: 1616, wherein the CDRs are as defined above. In one embodiment, the ISVD specifically binding to PD-1 comprises or consists of the amino acid sequence of SEQ ID NO: 1616.

In another embodiment, the ISVD specifically binding to human PD-1 may have a sequence identity of more than 90%, such as more than 95% or even more than 99%, with SEQ ID NO: 1617, wherein the CDRs are as defined above. In one embodiment, the ISVD specifically binding to PD-1 comprises or consists of the amino acid sequence of SEQ ID NO: 1617.

In another embodiment, the ISVD specifically binding to human PD-1 may have a sequence identity of more than 90%, such as more than 95% or even more than 99%, with SEQ ID NO: 1618, wherein the CDRs are as defined above. In one embodiment, the ISVD specifically binding to PD-1 comprises or consists of the amino acid sequence of SEQ ID NO: 1618.

In another embodiment, the ISVD specifically binding to human PD-1 may have a sequence identity of more than 90%, such as more than 95% or even more than 99%, with SEQ ID NO: 1619, wherein the CDRs are as defined above. In one embodiment, the ISVD specifically binding to PD-1 comprises or consists of the amino acid sequence of SEQ ID NO: 1619.

In another embodiment, the ISVD specifically binding to human PD-1 may have a sequence identity of more than 90%, such as more than 95% or even more than 99%, with SEQ ID NO: 1620, wherein the CDRs are as defined above. In one embodiment, the ISVD specifically binding to PD-1 comprises or consists of the amino acid sequence of SEQ ID NO: 1620.

In another embodiment, the ISVD specifically binding to human PD-1 may have a sequence identity of more than 90%, such as more than 95% or even more than 99%, with SEQ ID NO: 1621, wherein the CDRs are as defined above. In one embodiment, the ISVD specifically binding to PD-1 comprises or consists of the amino acid sequence of SEQ ID NO: 1621.

In another embodiment, the ISVD specifically binding to human PD-1 may have a sequence identity of more than 90%, such as more than 95% or even more than 99%, with SEQ ID NO: 1622, wherein the CDRs are as defined above. In one embodiment, the ISVD specifically binding to PD-1 comprises or consists of the amino acid sequence of SEQ ID NO: 1622.

In another embodiment, the ISVD specifically binding to human PD-1 may have a sequence identity of more than 90%, such as more than 95% or even more than 99%, with SEQ ID NO: 1623, wherein the CDRs are as defined above. In one embodiment, the ISVD specifically binding to PD-1 comprises or consists of the amino acid sequence of SEQ ID NO: 1623.

In another embodiment, the ISVD specifically binding to human PD-1 may have a sequence identity of more than 90%, such as more than 95% or even more than 99%, with SEQ ID NO: 1624, wherein the CDRs are as defined above. In one embodiment, the ISVD specifically binding to PD-1 comprises or consists of the amino acid sequence of SEQ ID NO: 1624.

In another embodiment, the ISVD specifically binding to human PD-1 may have a sequence identity of more than 90%, such as more than 95% or even more than 99%, with SEQ ID NO: 1625, wherein the CDRs are as defined above. In one embodiment, the ISVD specifically binding to PD-1 comprises or consists of the amino acid sequence of SEQ ID NO: 1625.

In another embodiment, the ISVD specifically binding to human PD-1 may have a sequence identity of more than 90%, such as more than 95% or even more than 99%, with SEQ ID NO: 1626, wherein the CDRs are as defined above. In one embodiment, the ISVD specifically binding to PD-1 comprises or consists of the amino acid sequence of SEQ ID NO: 1626.

In another embodiment, the ISVD specifically binding to human PD-1 may have a sequence identity of more than 90%, such as more than 95% or even more than 99%, with SEQ ID NO: 1627, wherein the CDRs are as defined above. In one embodiment, the ISVD specifically binding to PD-1 comprises or consists of the amino acid sequence of SEQ ID NO: 1627.

In another embodiment, the ISVD specifically binding to human PD-1 may have a sequence identity of more than 90%, such as more than 95% or even more than 99%, with SEQ ID NO: 1628, wherein the CDRs are as defined above. In one embodiment, the ISVD specifically binding to PD-1 comprises or consists of the amino acid sequence of SEQ ID NO: 1628.

In another embodiment, the ISVD specifically binding to human PD-1 may have a sequence identity of more than 90%, such as more than 95% or even more than 99%, with SEQ ID NO: 1629, wherein the CDRs are as defined above. In one embodiment, the ISVD specifically binding to PD-1 comprises or consists of the amino acid sequence of SEQ ID NO: 1629.

In another embodiment, the ISVD specifically binding to human PD-1 may have a sequence identity of more than 90%, such as more than 95% or even more than 99%, with SEQ ID NO: 1630, wherein the CDRs are as defined above. In one embodiment, the ISVD specifically binding to PD-1 comprises or consists of the amino acid sequence of SEQ ID NO: 1630.

In another embodiment, the ISVD specifically binding to human PD-1 may have a sequence identity of more than 90%, such as more than 95% or even more than 99%, with SEQ ID NO: 1631, wherein the CDRs are as defined above. In one embodiment, the ISVD specifically binding to PD-1 comprises or consists of the amino acid sequence of SEQ ID NO: 1631.

In another embodiment, the ISVD specifically binding to human PD-1 may have a sequence identity of more than 90%, such as more than 95% or even more than 99%, with SEQ ID NO: 1632, wherein the CDRs are as defined above. In one embodiment, the ISVD specifically binding to PD-1 comprises or consists of the amino acid sequence of SEQ ID NO: 1632.

In another embodiment, the ISVD specifically binding to human PD-1 may have a sequence identity of more than 90%, such as more than 95% or even more than 99%, with SEQ ID NO: 1633, wherein the CDRs are as defined above. In one embodiment, the ISVD specifically binding to PD-1 comprises or consists of the amino acid sequence of SEQ ID NO: 1633.

In another embodiment, the ISVD specifically binding to human PD-1 may have a sequence identity of more than 90%, such as more than 95% or even more than 99%, with SEQ ID NO: 1634, wherein the CDRs are as defined above. In one embodiment, the ISVD specifically binding to PD-1 comprises or consists of the amino acid sequence of SEQ ID NO: 1634.

In another embodiment, the ISVD specifically binding to human PD-1 may have a sequence identity of more than 90%, such as more than 95% or even more than 99%, with SEQ ID NO: 1635, wherein the CDRs are as defined above. In one embodiment, the ISVD specifically binding to PD-1 comprises or consists of the amino acid sequence of SEQ ID NO: 1635.

In another embodiment, the ISVD specifically binding to human PD-1 may have a sequence identity of more than 90%, such as more than 95% or even more than 99%, with SEQ ID NO: 1636, wherein the CDRs are as defined above. In one embodiment, the ISVD specifically binding to PD-1 comprises or consists of the amino acid sequence of SEQ ID NO: 1636.

In another embodiment, the ISVD specifically binding to human PD-1 may have a sequence identity of more than 90%, such as more than 95% or even more than 99%, with SEQ ID NO: 1637, wherein the CDRs are as defined above. In one embodiment, the ISVD specifically binding to PD-1 comprises or consists of the amino acid sequence of SEQ ID NO: 1637.

In another embodiment, the ISVD specifically binding to human PD-1 may have a sequence identity of more than 90%, such as more than 95% or even more than 99%, with SEQ ID NO: 1638, wherein the CDRs are as defined above. In one embodiment, the ISVD specifically binding to PD-1 comprises or consists of the amino acid sequence of SEQ ID NO: 1638.

In another embodiment, the ISVD specifically binding to human PD-1 may have a sequence identity of more than 90%, such as more than 95% or even more than 99%, with SEQ ID NO: 1639, wherein the CDRs are as defined above. In one embodiment, the ISVD specifically binding to PD-1 comprises or consists of the amino acid sequence of SEQ ID NO: 1639.

In another embodiment, the ISVD specifically binding to human PD-1 may have a sequence identity of more than 90%, such as more than 95% or even more than 99%, with SEQ ID NO: 1640, wherein the CDRs are as defined above. In one embodiment, the ISVD specifically binding to PD-1 comprises or consists of the amino acid sequence of SEQ ID NO: 1640.

In another embodiment, the ISVD specifically binding to human PD-1 may have a sequence identity of more than 90%, such as more than 95% or even more than 99%, with SEQ ID NO: 1641, wherein the CDRs are as defined above. In one embodiment, the ISVD specifically binding to PD-1 comprises or consists of the amino acid sequence of SEQ ID NO: 1641.

In another embodiment, the ISVD specifically binding to human PD-1 may have a sequence identity of more than 90%, such as more than 95% or even more than 99%, with SEQ ID NO: 1642, wherein the CDRs are as defined above. In one embodiment, the ISVD specifically binding to PD-1 comprises or consists of the amino acid sequence of SEQ ID NO: 1642.

In another embodiment, the ISVD specifically binding to human PD-1 may have a sequence identity of more than 90%, such as more than 95% or even more than 99%, with SEQ ID NO: 1643, wherein the CDRs are as defined above. In one embodiment, the ISVD specifically binding to PD-1 comprises or consists of the amino acid sequence of SEQ ID NO: 1643.

In another embodiment, the ISVD specifically binding to human PD-1 may have a sequence identity of more than 90%, such as more than 95% or even more than 99%, with SEQ ID NO: 1644, wherein the CDRs are as defined above. In one embodiment, the ISVD specifically binding to PD-1 comprises or consists of the amino acid sequence of SEQ ID NO: 1644.

In another embodiment, the ISVD specifically binding to human PD-1 may have a sequence identity of more than 90%, such as more than 95% or even more than 99%, with SEQ ID NO: 1645, wherein the CDRs are as defined above. In one embodiment, the ISVD specifically binding to PD-1 comprises or consists of the amino acid sequence of SEQ ID NO: 1645.

In another embodiment, the ISVD specifically binding to human PD-1 may have a sequence identity of more than 90%, such as more than 95% or even more than 99%, with SEQ ID NO: 1646, wherein the CDRs are as defined above. In one embodiment, the ISVD specifically binding to PD-1 comprises or consists of the amino acid sequence of SEQ ID NO: 1646.

In another embodiment, the ISVD specifically binding to human PD-1 may have a sequence identity of more than 90%, such as more than 95% or even more than 99%, with SEQ ID NO: 1647, wherein the CDRs are as defined above. In one embodiment, the ISVD specifically binding to PD-1 comprises or consists of the amino acid sequence of SEQ ID NO: 1647.

In another embodiment, the ISVD specifically binding to human PD-1 may have a sequence identity of more than 90%, such as more than 95% or even more than 99%, with SEQ ID NO: 1648, wherein the CDRs are as defined above. In one embodiment, the ISVD specifically binding to PD-1 comprises or consists of the amino acid sequence of SEQ ID NO: 1648.

In another embodiment, the ISVD specifically binding to human PD-1 may have a sequence identity of more than 90%, such as more than 95% or even more than 99%, with SEQ ID NO: 1649, wherein the CDRs are as defined above. In one embodiment, the ISVD specifically binding to PD-1 comprises or consists of the amino acid sequence of SEQ ID NO: 1649.

In another embodiment, the ISVD specifically binding to human PD-1 may have a sequence identity of more than 90%, such as more than 95% or even more than 99%, with SEQ ID NO: 1650, wherein the CDRs are as defined above. In one embodiment, the ISVD specifically binding to PD-1 comprises or consists of the amino acid sequence of SEQ ID NO: 1650.

In another embodiment, the ISVD specifically binding to human PD-1 may have a sequence identity of more than 90%, such as more than 95% or even more than 99%, with SEQ ID NO: 1651, wherein the CDRs are as defined above. In one embodiment, the ISVD specifically binding to PD-1 comprises or consists of the amino acid sequence of SEQ ID NO: 1651.

In another embodiment, the ISVD specifically binding to human PD-1 may have a sequence identity of more than 90%, such as more than 95% or even more than 99%, with SEQ ID NO: 1652, wherein the CDRs are as defined above. In one embodiment, the ISVD specifically binding to PD-1 comprises or consists of the amino acid sequence of SEQ ID NO: 1652.

In another embodiment, the ISVD specifically binding to human PD-1 may have a sequence identity of more than 90%, such as more than 95% or even more than 99%, with SEQ ID NO: 1653, wherein the CDRs are as defined above. In one embodiment, the ISVD specifically binding to PD-1 comprises or consists of the amino acid sequence of SEQ ID NO: 1653.

In another embodiment, the ISVD specifically binding to human PD-1 may have a sequence identity of more than 90%, such as more than 95% or even more than 99%, with SEQ ID NO: 1654, wherein the CDRs are as defined above. In one embodiment, the ISVD specifically binding to PD-1 comprises or consists of the amino acid sequence of SEQ ID NO: 1654.

In another embodiment, the ISVD specifically binding to human PD-1 may have a sequence identity of more than 90%, such as more than 95% or even more than 99%, with SEQ ID NO: 1655, wherein the CDRs are as defined above. In one embodiment, the ISVD specifically binding to PD-1 comprises or consists of the amino acid sequence of SEQ ID NO: 1655.

In another embodiment, the ISVD specifically binding to human PD-1 may have a sequence identity of more than 90%, such as more than 95% or even more than 99%, with SEQ ID NO: 1656, wherein the CDRs are as defined above. In one embodiment, the ISVD specifically binding to PD-1 comprises or consists of the amino acid sequence of SEQ ID NO: 1656.

In another embodiment, the ISVD specifically binding to human PD-1 may have a sequence identity of more than 90%, such as more than 95% or even more than 99%, with SEQ ID NO: 1657, wherein the CDRs are as defined above. In one embodiment, the ISVD specifically binding to PD-1 comprises or consists of the amino acid sequence of SEQ ID NO: 1657.

In another embodiment, the ISVD specifically binding to human PD-1 may have a sequence identity of more than 90%, such as more than 95% or even more than 99%, with SEQ ID NO: 1658, wherein the CDRs are as defined above. In one embodiment, the ISVD specifically binding to PD-1 comprises or consists of the amino acid sequence of SEQ ID NO: 1658.

In another embodiment, the ISVD specifically binding to human PD-1 may have a sequence identity of more than 90%, such as more than 95% or even more than 99%, with SEQ ID NO: 1659, wherein the CDRs are as defined above. In one embodiment, the ISVD specifically binding to PD-1 comprises or consists of the amino acid sequence of SEQ ID NO: 1659.

In another embodiment, the ISVD specifically binding to human PD-1 may have a sequence identity of more than 90%, such as more than 95% or even more than 99%, with SEQ ID NO: 1660, wherein the CDRs are as defined above. In one embodiment, the ISVD specifically binding to PD-1 comprises or consists of the amino acid sequence of SEQ ID NO: 1660.

In another embodiment, the ISVD specifically binding to human PD-1 may have a sequence identity of more than 90%, such as more than 95% or even more than 99%, with SEQ ID NO: 1661, wherein the CDRs are as defined above. In one embodiment, the ISVD specifically binding to PD-1 comprises or consists of the amino acid sequence of SEQ ID NO: 1661.

In another embodiment, the ISVD specifically binding to human PD-1 may have a sequence identity of more than 90%, such as more than 95% or even more than 99%, with SEQ ID NO: 1662, wherein the CDRs are as defined above. In one embodiment, the ISVD specifically binding to PD-1 comprises or consists of the amino acid sequence of SEQ ID NO: 1662.

In another embodiment, the ISVD specifically binding to human PD-1 may have a sequence identity of more than 90%, such as more than 95% or even more than 99%, with SEQ ID NO: 1663, wherein the CDRs are as defined above. In one embodiment, the ISVD specifically binding to PD-1 comprises or consists of the amino acid sequence of SEQ ID NO: 1663.

In another embodiment, the ISVD specifically binding to human PD-1 may have a sequence identity of more than 90%, such as more than 95% or even more than 99%, with SEQ ID NO: 1664, wherein the CDRs are as defined above. In one embodiment, the ISVD specifically binding to PD-1 comprises or consists of the amino acid sequence of SEQ ID NO: 1664.

In another embodiment, the ISVD specifically binding to human PD-1 may have a sequence identity of more than 90%, such as more than 95% or even more than 99%, with SEQ ID NO: 1665, wherein the CDRs are as defined above. In one embodiment, the ISVD specifically binding to PD-1 comprises or consists of the amino acid sequence of SEQ ID NO: 1665.

In another embodiment, the ISVD specifically binding to human PD-1 may have a sequence identity of more than 90%, such as more than 95% or even more than 99%, with SEQ ID NO: 1666, wherein the CDRs are as defined above. In one embodiment, the ISVD specifically binding to PD-1 comprises or consists of the amino acid sequence of SEQ ID NO: 1666.

In another embodiment, the ISVD specifically binding to human PD-1 may have a sequence identity of more than 90%, such as more than 95% or even more than 99%, with SEQ ID NO: 1667, wherein the CDRs are as defined above. In one embodiment, the ISVD specifically binding to PD-1 comprises or consists of the amino acid sequence of SEQ ID NO: 1667.

In another embodiment, the ISVD specifically binding to human PD-1 may have a sequence identity of more than 90%, such as more than 95% or even more than 99%, with SEQ ID NO: 1668, wherein the CDRs are as defined above. In one embodiment, the ISVD specifically binding to PD-1 comprises or consists of the amino acid sequence of SEQ ID NO: 1668.

In another embodiment, the ISVD specifically binding to human PD-1 may have a sequence identity of more than 90%, such as more than 95% or even more than 99%, with SEQ ID NO: 1669, wherein the CDRs are as defined above. In one embodiment, the ISVD specifically binding to PD-1 comprises or consists of the amino acid sequence of SEQ ID NO: 1669.

In another embodiment, the ISVD specifically binding to human PD-1 may have a sequence identity of more than 90%, such as more than 95% or even more than 99%, with SEQ ID NO: 1670, wherein the CDRs are as defined above. In one embodiment, the ISVD specifically binding to PD-1 comprises or consists of the amino acid sequence of SEQ ID NO: 1670.

In another embodiment, the ISVD specifically binding to human PD-1 may have a sequence identity of more than 90%, such as more than 95% or even more than 99%, with SEQ ID NO: 1671, wherein the CDRs are as defined above. In one embodiment, the ISVD specifically binding to PD-1 comprises or consists of the amino acid sequence of SEQ ID NO: 1671.

In another embodiment, the ISVD specifically binding to human PD-1 may have a sequence identity of more than 90%, such as more than 95% or even more than 99%, with SEQ ID NO: 1672, wherein the CDRs are as defined above. In one embodiment, the ISVD specifically binding to PD-1 comprises or consists of the amino acid sequence of SEQ ID NO: 1672.

In another embodiment, the ISVD specifically binding to human PD-1 may have a sequence identity of more than 90%, such as more than 95% or even more than 99%, with SEQ ID NO: 1673, wherein the CDRs are as defined above. In one embodiment, the ISVD specifically binding to PD-1 comprises or consists of the amino acid sequence of SEQ ID NO: 1673.

In another embodiment, the ISVD specifically binding to human PD-1 may have a sequence identity of more than 90%, such as more than 95% or even more than 99%, with SEQ ID NO: 1674, wherein the CDRs are as defined above. In one embodiment, the ISVD specifically binding to PD-1 comprises or consists of the amino acid sequence of SEQ ID NO: 1674.

In another embodiment, the ISVD specifically binding to human PD-1 may have a sequence identity of more than 90%, such as more than 95% or even more than 99%, with SEQ ID NO: 1675, wherein the CDRs are as defined above. In one embodiment, the ISVD specifically binding to PD-1 comprises or consists of the amino acid sequence of SEQ ID NO: 1675.

In another embodiment, the ISVD specifically binding to human PD-1 may have a sequence identity of more than 90%, such as more than 95% or even more than 99%, with SEQ ID NO: 1676, wherein the CDRs are as defined above. In one embodiment, the ISVD specifically binding to PD-1 comprises or consists of the amino acid sequence of SEQ ID NO: 1676.

In another embodiment, the ISVD specifically binding to human PD-1 may have a sequence identity of more than 90%, such as more than 95% or even more than 99%, with SEQ ID NO: 1677, wherein the CDRs are as defined above. In one embodiment, the ISVD specifically binding to PD-1 comprises or consists of the amino acid sequence of SEQ ID NO: 1677.

In another embodiment, the ISVD specifically binding to human PD-1 may have a sequence identity of more than 90%, such as more than 95% or even more than 99%, with SEQ ID NO: 1678, wherein the CDRs are as defined above. In one embodiment, the ISVD specifically binding to PD-1 comprises or consists of the amino acid sequence of SEQ ID NO: 1678.

In another embodiment, the ISVD specifically binding to human PD-1 may have a sequence identity of more than 90%, such as more than 95% or even more than 99%, with SEQ ID NO: 1679, wherein the CDRs are as defined above. In one embodiment, the ISVD specifically binding to PD-1 comprises or consists of the amino acid sequence of SEQ ID NO: 1679.

In another embodiment, the ISVD specifically binding to human PD-1 may have a sequence identity of more than 90%, such as more than 95% or even more than 99%, with SEQ ID NO: 1680, wherein the CDRs are as defined above. In one embodiment, the ISVD specifically binding to PD-1 comprises or consists of the amino acid sequence of SEQ ID NO: 1680.

In another embodiment, the ISVD specifically binding to human PD-1 may have a sequence identity of more than 90%, such as more than 95% or even more than 99%, with SEQ ID NO: 1681, wherein the CDRs are as defined above. In one embodiment, the ISVD specifically binding to PD-1 comprises or consists of the amino acid sequence of SEQ ID NO: 1681.

In another embodiment, the ISVD specifically binding to human PD-1 may have a sequence identity of more than 90%, such as more than 95% or even more than 99%, with SEQ ID NO: 1682, wherein the CDRs are as defined above. In one embodiment, the ISVD specifically binding to PD-1 comprises or consists of the amino acid sequence of SEQ ID NO: 1682.

In another embodiment, the ISVD specifically binding to human PD-1 may have a sequence identity of more than 90%, such as more than 95% or even more than 99%, with SEQ ID NO: 1683, wherein the CDRs are as defined above. In one embodiment, the ISVD specifically binding to PD-1 comprises or consists of the amino acid sequence of SEQ ID NO: 1683.

In another embodiment, the ISVD specifically binding to human PD-1 may have a sequence identity of more than 90%, such as more than 95% or even more than 99%, with SEQ ID NO: 1684, wherein the CDRs are as defined above. In one embodiment, the ISVD specifically binding to PD-1 comprises or consists of the amino acid sequence of SEQ ID NO: 1684.

In another embodiment, the ISVD specifically binding to human PD-1 may have a sequence identity of more than 90%, such as more than 95% or even more than 99%, with SEQ ID NO: 1685, wherein the CDRs are as defined above. In one embodiment, the ISVD specifically binding to PD-1 comprises or consists of the amino acid sequence of SEQ ID NO: 1685.

In another embodiment, the ISVD specifically binding to human PD-1 may have a sequence identity of more than 90%, such as more than 95% or even more than 99%, with SEQ ID NO: 1686, wherein the CDRs are as defined above. In one embodiment, the ISVD specifically binding to PD-1 comprises or consists of the amino acid sequence of SEQ ID NO: 1686.

In another embodiment, the ISVD specifically binding to human PD-1 may have a sequence identity of more than 90%, such as more than 95% or even more than 99%, with SEQ ID NO: 1687, wherein the CDRs are as defined above. In one embodiment, the ISVD specifically binding to PD-1 comprises or consists of the amino acid sequence of SEQ ID NO: 1687.

In another embodiment, the ISVD specifically binding to human PD-1 may have a sequence identity of more than 90%, such as more than 95% or even more than 99%, with SEQ ID NO: 1688, wherein the CDRs are as defined above. In one embodiment, the ISVD specifically binding to PD-1 comprises or consists of the amino acid sequence of SEQ ID NO: 1688.

In another embodiment, the ISVD specifically binding to human PD-1 may have a sequence identity of more than 90%, such as more than 95% or even more than 99%, with SEQ ID NO: 1689, wherein the CDRs are as defined above. In one embodiment, the ISVD specifically binding to PD-1 comprises or consists of the amino acid sequence of SEQ ID NO: 1689.

In another embodiment, the ISVD specifically binding to human PD-1 may have a sequence identity of more than 90%, such as more than 95% or even more than 99%, with SEQ ID NO: 1690, wherein the CDRs are as defined above. In one embodiment, the ISVD specifically binding to PD-1 comprises or consists of the amino acid sequence of SEQ ID NO: 1690.

In another embodiment, the ISVD specifically binding to human PD-1 may have a sequence identity of more than 90%, such as more than 95% or even more than 99%, with SEQ ID NO: 1691, wherein the CDRs are as defined above. In one embodiment, the ISVD specifically binding to PD-1 comprises or consists of the amino acid sequence of SEQ ID NO: 1691.

In another embodiment, the ISVD specifically binding to human PD-1 may have a sequence identity of more than 90%, such as more than 95% or even more than 99%, with SEQ ID NO: 1692, wherein the CDRs are as defined above. In one embodiment, the ISVD specifically binding to PD-1 comprises or consists of the amino acid sequence of SEQ ID NO: 1692.

In another embodiment, the ISVD specifically binding to human PD-1 may have a sequence identity of more than 90%, such as more than 95% or even more than 99%, with SEQ ID NO: 1693, wherein the CDRs are as defined above. In one embodiment, the ISVD specifically binding to PD-1 comprises or consists of the amino acid sequence of SEQ ID NO: 1693.

In another embodiment, the ISVD specifically binding to human PD-1 may have a sequence identity of more than 90%, such as more than 95% or even more than 99%, with SEQ ID NO: 1694, wherein the CDRs are as defined above. In one embodiment, the ISVD specifically binding to PD-1 comprises or consists of the amino acid sequence of SEQ ID NO: 1694.

In another embodiment, the ISVD specifically binding to human PD-1 may have a sequence identity of more than 90%, such as more than 95% or even more than 99%, with SEQ ID NO: 1695, wherein the CDRs are as defined above. In one embodiment, the ISVD specifically binding to PD-1 comprises or consists of the amino acid sequence of SEQ ID NO: 1695.

In another embodiment, the ISVD specifically binding to human PD-1 may have a sequence identity of more than 90%, such as more than 95% or even more than 99%, with SEQ ID NO: 1696, wherein the CDRs are as defined above. In one embodiment, the ISVD specifically binding to PD-1 comprises or consists of the amino acid sequence of SEQ ID NO: 1696.

In another embodiment, the ISVD specifically binding to human PD-1 may have a sequence identity of more than 90%, such as more than 95% or even more than 99%, with SEQ ID NO: 1697, wherein the CDRs are as defined above. In one embodiment, the ISVD specifically binding to PD-1 comprises or consists of the amino acid sequence of SEQ ID NO: 1697.

In another embodiment, the ISVD specifically binding to human PD-1 may have a sequence identity of more than 90%, such as more than 95% or even more than 99%, with SEQ ID NO: 1698, wherein the CDRs are as defined above. In one embodiment, the ISVD specifically binding to PD-1 comprises or consists of the amino acid sequence of SEQ ID NO: 1698.

In another embodiment, the ISVD specifically binding to human PD-1 may have a sequence identity of more than 90%, such as more than 95% or even more than 99%, with SEQ ID NO: 1699, wherein the CDRs are as defined above. In one embodiment, the ISVD specifically binding to PD-1 comprises or consists of the amino acid sequence of SEQ ID NO: 1699.

In another embodiment, the ISVD specifically binding to human PD-1 may have a sequence identity of more than 90%, such as more than 95% or even more than 99%, with SEQ ID NO: 1700, wherein the CDRs are as defined above. In one embodiment, the ISVD specifically binding to PD-1 comprises or consists of the amino acid sequence of SEQ ID NO: 1700.

In another embodiment, the ISVD specifically binding to human PD-1 may have a sequence identity of more than 90%, such as more than 95% or even more than 99%, with SEQ ID NO: 1701, wherein the CDRs are as defined above. In one embodiment, the ISVD specifically binding to PD-1 comprises or consists of the amino acid sequence of SEQ ID NO: 1701.

In another embodiment, the ISVD specifically binding to human PD-1 may have a sequence identity of more than 90%, such as more than 95% or even more than 99%, with SEQ ID NO: 1702, wherein the CDRs are as defined above. In one embodiment, the ISVD specifically binding to PD-1 comprises or consists of the amino acid sequence of SEQ ID NO: 1702.

In another embodiment, the ISVD specifically binding to human PD-1 may have a sequence identity of more than 90%, such as more than 95% or even more than 99%, with SEQ ID NO: 1703, wherein the CDRs are as defined above. In one embodiment, the ISVD specifically binding to PD-1 comprises or consists of the amino acid sequence of SEQ ID NO: 1703.

In another embodiment, the ISVD specifically binding to human PD-1 may have a sequence identity of more than 90%, such as more than 95% or even more than 99%, with SEQ ID NO: 1704, wherein the CDRs are as defined above. In one embodiment, the ISVD specifically binding to PD-1 comprises or consists of the amino acid sequence of SEQ ID NO: 1704.

In another embodiment, the ISVD specifically binding to human PD-1 may have a sequence identity of more than 90%, such as more than 95% or even more than 99%, with SEQ ID NO: 1705, wherein the CDRs are as defined above. In one embodiment, the ISVD specifically binding to PD-1 comprises or consists of the amino acid sequence of SEQ ID NO: 1705.

In another embodiment, the ISVD specifically binding to human PD-1 may have a sequence identity of more than 90%, such as more than 95% or even more than 99%, with SEQ ID NO: 1706, wherein the CDRs are as defined above. In one embodiment, the ISVD specifically binding to PD-1 comprises or consists of the amino acid sequence of SEQ ID NO: 1706.

In another embodiment, the ISVD specifically binding to human PD-1 may have a sequence identity of more than 90%, such as more than 95% or even more than 99%, with SEQ ID NO: 1707, wherein the CDRs are as defined above. In one embodiment, the ISVD specifically binding to PD-1 comprises or consists of the amino acid sequence of SEQ ID NO: 1707.

In another embodiment, the ISVD specifically binding to human PD-1 may have a sequence identity of more than 90%, such as more than 95% or even more than 99%, with SEQ ID NO: 1708, wherein the CDRs are as defined above. In one embodiment, the ISVD specifically binding to PD-1 comprises or consists of the amino acid sequence of SEQ ID NO: 1708.

In another embodiment, the ISVD specifically binding to human PD-1 may have a sequence identity of more than 90%, such as more than 95% or even more than 99%, with SEQ ID NO: 1709, wherein the CDRs are as defined above. In one embodiment, the ISVD specifically binding to PD-1 comprises or consists of the amino acid sequence of SEQ ID NO: 1709.

In another embodiment, the ISVD specifically binding to human PD-1 may have a sequence identity of more than 90%, such as more than 95% or even more than 99%, with SEQ ID NO: 1710, wherein the CDRs are as defined above. In one embodiment, the ISVD specifically binding to PD-1 comprises or consists of the amino acid sequence of SEQ ID NO: 1710.

In another embodiment, the ISVD specifically binding to human PD-1 may have a sequence identity of more than 90%, such as more than 95% or even more than 99%, with SEQ ID NO: 1711, wherein the CDRs are as defined above. In one embodiment, the ISVD specifically binding to PD-1 comprises or consists of the amino acid sequence of SEQ ID NO: 1711.

In another embodiment, the ISVD specifically binding to human PD-1 may have a sequence identity of more than 90%, such as more than 95% or even more than 99%, with SEQ ID NO: 1712, wherein the CDRs are as defined above. In one embodiment, the ISVD specifically binding to PD-1 comprises or consists of the amino acid sequence of SEQ ID NO: 1712.

In another embodiment, the ISVD specifically binding to human PD-1 may have a sequence identity of more than 90%, such as more than 95% or even more than 99%, with SEQ ID NO: 1713, wherein the CDRs are as defined above. In one embodiment, the ISVD specifically binding to PD-1 comprises or consists of the amino acid sequence of SEQ ID NO: 1713.

In another embodiment, the ISVD specifically binding to human PD-1 may have a sequence identity of more than 90%, such as more than 95% or even more than 99%, with SEQ ID NO: 1714, wherein the CDRs are as defined above. In one embodiment, the ISVD specifically binding to PD-1 comprises or consists of the amino acid sequence of SEQ ID NO: 1714.

In another embodiment, the ISVD specifically binding to human PD-1 may have a sequence identity of more than 90%, such as more than 95% or even more than 99%, with SEQ ID NO: 1715, wherein the CDRs are as defined above. In one embodiment, the ISVD specifically binding to PD-1 comprises or consists of the amino acid sequence of SEQ ID NO: 1715.

In another embodiment, the ISVD specifically binding to human PD-1 may have a sequence identity of more than 90%, such as more than 95% or even more than 99%, with SEQ ID NO: 1716, wherein the CDRs are as defined above. In one embodiment, the ISVD specifically binding to PD-1 comprises or consists of the amino acid sequence of SEQ ID NO: 1716.

In another embodiment, the ISVD specifically binding to human PD-1 may have a sequence identity of more than 90%, such as more than 95% or even more than 99%, with SEQ ID NO: 1717, wherein the CDRs are as defined above. In one embodiment, the ISVD specifically binding to PD-1 comprises or consists of the amino acid sequence of SEQ ID NO: 1717.

In another embodiment, the ISVD specifically binding to human PD-1 may have a sequence identity of more than 90%, such as more than 95% or even more than 99%, with SEQ ID NO: 1718, wherein the CDRs are as defined above. In one embodiment, the ISVD specifically binding to PD-1 comprises or consists of the amino acid sequence of SEQ ID NO: 1718.

In another embodiment, the ISVD specifically binding to human PD-1 may have a sequence identity of more than 90%, such as more than 95% or even more than 99%, with SEQ ID NO: 1719, wherein the CDRs are as defined above. In one embodiment, the ISVD specifically binding to PD-1 comprises or consists of the amino acid sequence of SEQ ID NO: 1719.

In another embodiment, the ISVD specifically binding to human PD-1 may have a sequence identity of more than 90%, such as more than 95% or even more than 99%, with SEQ ID NO: 1720, wherein the CDRs are as defined above. In one embodiment, the ISVD specifically binding to PD-1 comprises or consists of the amino acid sequence of SEQ ID NO: 1720.

In another embodiment, the ISVD specifically binding to human PD-1 may have a sequence identity of more than 90%, such as more than 95% or even more than 99%, with SEQ ID NO: 1721, wherein the CDRs are as defined above. In one embodiment, the ISVD specifically binding to PD-1 comprises or consists of the amino acid sequence of SEQ ID NO: 1721.

In another embodiment, the ISVD specifically binding to human PD-1 may have a sequence identity of more than 90%, such as more than 95% or even more than 99%, with SEQ ID NO: 1722, wherein the CDRs are as defined above. In one embodiment, the ISVD specifically binding to PD-1 comprises or consists of the amino acid sequence of SEQ ID NO: 1722.

In another embodiment, the ISVD specifically binding to human PD-1 may have a sequence identity of more than 90%, such as more than 95% or even more than 99%, with SEQ ID NO: 1723, wherein the CDRs are as defined above. In one embodiment, the ISVD specifically binding to PD-1 comprises or consists of the amino acid sequence of SEQ ID NO: 1723.

In another embodiment, the ISVD specifically binding to human PD-1 may have a sequence identity of more than 90%, such as more than 95% or even more than 99%, with SEQ ID NO: 1724, wherein the CDRs are as defined above. In one embodiment, the ISVD specifically binding to PD-1 comprises or consists of the amino acid sequence of SEQ ID NO: 1724.

In another embodiment, the ISVD specifically binding to human PD-1 may have a sequence identity of more than 90%, such as more than 95% or even more than 99%, with SEQ ID NO: 1725, wherein the CDRs are as defined above. In one embodiment, the ISVD specifically binding to PD-1 comprises or consists of the amino acid sequence of SEQ ID NO: 1725.

In another embodiment, the ISVD specifically binding to human PD-1 may have a sequence identity of more than 90%, such as more than 95% or even more than 99%, with SEQ ID NO: 1726, wherein the CDRs are as defined above. In one embodiment, the ISVD specifically binding to PD-1 comprises or consists of the amino acid sequence of SEQ ID NO: 1726.

In another embodiment, the ISVD specifically binding to human PD-1 may have a sequence identity of more than 90%, such as more than 95% or even more than 99%, with SEQ ID NO: 1727, wherein the CDRs are as defined above. In one embodiment, the ISVD specifically binding to PD-1 comprises or consists of the amino acid sequence of SEQ ID NO: 1727.

In another embodiment, the ISVD specifically binding to human PD-1 may have a sequence identity of more than 90%, such as more than 95% or even more than 99%, with SEQ ID NO: 1728, wherein the CDRs are as defined above. In one embodiment, the ISVD specifically binding to PD-1 comprises or consists of the amino acid sequence of SEQ ID NO: 1728.

In another embodiment, the ISVD specifically binding to human PD-1 may have a sequence identity of more than 90%, such as more than 95% or even more than 99%, with SEQ ID NO: 1729, wherein the CDRs are as defined above. In one embodiment, the ISVD specifically binding to PD-1 comprises or consists of the amino acid sequence of SEQ ID NO: 1729.

In another embodiment, the ISVD specifically binding to human PD-1 may have a sequence identity of more than 90%, such as more than 95% or even more than 99%, with SEQ ID NO: 1730, wherein the CDRs are as defined above. In one embodiment, the ISVD specifically binding to PD-1 comprises or consists of the amino acid sequence of SEQ ID NO: 1730.

In another embodiment, the ISVD specifically binding to human PD-1 may have a sequence identity of more than 90%, such as more than 95% or even more than 99%, with SEQ ID NO: 1731, wherein the CDRs are as defined above. In one embodiment, the ISVD specifically binding to PD-1 comprises or consists of the amino acid sequence of SEQ ID NO: 1731.

In another embodiment, the ISVD specifically binding to human PD-1 may have a sequence identity of more than 90%, such as more than 95% or even more than 99%, with SEQ ID NO: 1732, wherein the CDRs are as defined above. In one embodiment, the ISVD specifically binding to PD-1 comprises or consists of the amino acid sequence of SEQ ID NO: 1732.

In another embodiment, the ISVD specifically binding to human PD-1 may have a sequence identity of more than 90%, such as more than 95% or even more than 99%, with SEQ ID NO: 1733, wherein the CDRs are as defined above. In one embodiment, the ISVD specifically binding to PD-1 comprises or consists of the amino acid sequence of SEQ ID NO: 1733.

In another embodiment, the ISVD specifically binding to human PD-1 may have a sequence identity of more than 90%, such as more than 95% or even more than 99%, with SEQ ID NO: 1734, wherein the CDRs are as defined above. In one embodiment, the ISVD specifically binding to PD-1 comprises or consists of the amino acid sequence of SEQ ID NO: 1734.

In another embodiment, the ISVD specifically binding to human PD-1 may have a sequence identity of more than 90%, such as more than 95% or even more than 99%, with SEQ ID NO: 1735, wherein the CDRs are as defined above. In one embodiment, the ISVD specifically binding to PD-1 comprises or consists of the amino acid sequence of SEQ ID NO: 1735.

In another embodiment, the ISVD specifically binding to human PD-1 may have a sequence identity of more than 90%, such as more than 95% or even more than 99%, with SEQ ID NO: 1736, wherein the CDRs are as defined above. In one embodiment, the ISVD specifically binding to PD-1 comprises or consists of the amino acid sequence of SEQ ID NO: 1736.

In another embodiment, the ISVD specifically binding to human PD-1 may have a sequence identity of more than 90%, such as more than 95% or even more than 99%, with SEQ ID NO: 1737, wherein the CDRs are as defined above. In one embodiment, the ISVD specifically binding to PD-1 comprises or consists of the amino acid sequence of SEQ ID NO: 1737.

In another embodiment, the ISVD specifically binding to human PD-1 may have a sequence identity of more than 90%, such as more than 95% or even more than 99%, with SEQ ID NO: 1738, wherein the CDRs are as defined above. In one embodiment, the ISVD specifically binding to PD-1 comprises or consists of the amino acid sequence of SEQ ID NO: 1738.

In another embodiment, the ISVD specifically binding to human PD-1 may have a sequence identity of more than 90%, such as more than 95% or even more than 99%, with SEQ ID NO: 1739, wherein the CDRs are as defined above. In one embodiment, the ISVD specifically binding to PD-1 comprises or consists of the amino acid sequence of SEQ ID NO: 1739.

In another embodiment, the ISVD specifically binding to human PD-1 may have a sequence identity of more than 90%, such as more than 95% or even more than 99%, with SEQ ID NO: 1740, wherein the CDRs are as defined above. In one embodiment, the ISVD specifically binding to PD-1 comprises or consists of the amino acid sequence of SEQ ID NO: 1740.

In another embodiment, the ISVD specifically binding to human PD-1 may have a sequence identity of more than 90%, such as more than 95% or even more than 99%, with SEQ ID NO: 1741, wherein the CDRs are as defined above. In one embodiment, the ISVD specifically binding to PD-1 comprises or consists of the amino acid sequence of SEQ ID NO: 1741.

In another embodiment, the ISVD specifically binding to human PD-1 may have a sequence identity of more than 90%, such as more than 95% or even more than 99%, with SEQ ID NO: 1742, wherein the CDRs are as defined above. In one embodiment, the ISVD specifically binding to PD-1 comprises or consists of the amino acid sequence of SEQ ID NO: 1742.

In another embodiment, the ISVD specifically binding to human PD-1 may have a sequence identity of more than 90%, such as more than 95% or even more than 99%, with SEQ ID NO: 1743, wherein the CDRs are as defined above. In one embodiment, the ISVD specifically binding to PD-1 comprises or consists of the amino acid sequence of SEQ ID NO: 1743.

In another embodiment, the ISVD specifically binding to human PD-1 may have a sequence identity of more than 90%, such as more than 95% or even more than 99%, with SEQ ID NO: 1744, wherein the CDRs are as defined above. In one embodiment, the ISVD specifically binding to PD-1 comprises or consists of the amino acid sequence of SEQ ID NO: 1744.

In another embodiment, the ISVD specifically binding to human PD-1 may have a sequence identity of more than 90%, such as more than 95% or even more than 99%, with SEQ ID NO: 1745, wherein the CDRs are as defined above. In one embodiment, the ISVD specifically binding to PD-1 comprises or consists of the amino acid sequence of SEQ ID NO: 1745.

In another embodiment, the ISVD specifically binding to human PD-1 may have a sequence identity of more than 90%, such as more than 95% or even more than 99%, with SEQ ID NO: 1746, wherein the CDRs are as defined above. In one embodiment, the ISVD specifically binding to PD-1 comprises or consists of the amino acid sequence of SEQ ID NO: 1746.

In another embodiment, the ISVD specifically binding to human PD-1 may have a sequence identity of more than 90%, such as more than 95% or even more than 99%, with SEQ ID NO: 1747, wherein the CDRs are as defined above. In one embodiment, the ISVD specifically binding to PD-1 comprises or consists of the amino acid sequence of SEQ ID NO: 1747.

In another embodiment, the ISVD specifically binding to human PD-1 may have a sequence identity of more than 90%, such as more than 95% or even more than 99%, with SEQ ID NO: 1748, wherein the CDRs are as defined above. In one embodiment, the ISVD specifically binding to PD-1 comprises or consists of the amino acid sequence of SEQ ID NO: 1748.

In another embodiment, the ISVD specifically binding to human PD-1 may have a sequence identity of more than 90%, such as more than 95% or even more than 99%, with SEQ ID NO: 1749, wherein the CDRs are as defined above. In one embodiment, the ISVD specifically binding to PD-1 comprises or consists of the amino acid sequence of SEQ ID NO: 1749.

In another embodiment, the ISVD specifically binding to human PD-1 may have a sequence identity of more than 90%, such as more than 95% or even more than 99%, with SEQ ID NO: 1750, wherein the CDRs are as defined above. In one embodiment, the ISVD specifically binding to PD-1 comprises or consists of the amino acid sequence of SEQ ID NO: 1750.

In another embodiment, the ISVD specifically binding to human PD-1 may have a sequence identity of more than 90%, such as more than 95% or even more than 99%, with SEQ ID NO: 1751, wherein the CDRs are as defined above. In one embodiment, the ISVD specifically binding to PD-1 comprises or consists of the amino acid sequence of SEQ ID NO: 1751.

In another embodiment, the ISVD specifically binding to human PD-1 may have a sequence identity of more than 90%, such as more than 95% or even more than 99%, with SEQ ID NO: 1752, wherein the CDRs are as defined above. In one embodiment, the ISVD specifically binding to PD-1 comprises or consists of the amino acid sequence of SEQ ID NO: 1752.

In another embodiment, the ISVD specifically binding to human PD-1 may have a sequence identity of more than 90%, such as more than 95% or even more than 99%, with SEQ ID NO: 1753, wherein the CDRs are as defined above. In one embodiment, the ISVD specifically binding to PD-1 comprises or consists of the amino acid sequence of SEQ ID NO: 1753.

In another embodiment, the ISVD specifically binding to human PD-1 may have a sequence identity of more than 90%, such as more than 95% or even more than 99%, with SEQ ID NO: 1754, wherein the CDRs are as defined above. In one embodiment, the ISVD specifically binding to PD-1 comprises or consists of the amino acid sequence of SEQ ID NO: 1754.

In another embodiment, the ISVD specifically binding to human PD-1 may have a sequence identity of more than 90%, such as more than 95% or even more than 99%, with SEQ ID NO: 1755, wherein the CDRs are as defined above. In one embodiment, the ISVD specifically binding to PD-1 comprises or consists of the amino acid sequence of SEQ ID NO: 1755.

In another embodiment, the ISVD specifically binding to human PD-1 may have a sequence identity of more than 90%, such as more than 95% or even more than 99%, with SEQ ID NO: 1756, wherein the CDRs are as defined above. In one embodiment, the ISVD specifically binding to PD-1 comprises or consists of the amino acid sequence of SEQ ID NO: 1756.

In another embodiment, the ISVD specifically binding to human PD-1 may have a sequence identity of more than 90%, such as more than 95% or even more than 99%, with SEQ ID NO: 1757, wherein the CDRs are as defined above. In one embodiment, the ISVD specifically binding to PD-1 comprises or consists of the amino acid sequence of SEQ ID NO: 1757.

In another embodiment, the ISVD specifically binding to human PD-1 may have a sequence identity of more than 90%, such as more than 95% or even more than 99%, with SEQ ID NO: 1758, wherein the CDRs are as defined above. In one embodiment, the ISVD specifically binding to PD-1 comprises or consists of the amino acid sequence of SEQ ID NO: 1758.

In another embodiment, the ISVD specifically binding to human PD-1 may have a sequence identity of more than 90%, such as more than 95% or even more than 99%, with SEQ ID NO: 1759, wherein the CDRs are as defined above. In one embodiment, the ISVD specifically binding to PD-1 comprises or consists of the amino acid sequence of SEQ ID NO: 1759.

In another embodiment, the ISVD specifically binding to human PD-1 may have a sequence identity of more than 90%, such as more than 95% or even more than 99%, with SEQ ID NO: 1760, wherein the CDRs are as defined above. In one embodiment, the ISVD specifically binding to PD-1 comprises or consists of the amino acid sequence of SEQ ID NO: 1760.

In another embodiment, the ISVD specifically binding to human PD-1 may have a sequence identity of more than 90%, such as more than 95% or even more than 99%, with SEQ ID NO: 1761, wherein the CDRs are as defined above. In one embodiment, the ISVD specifically binding to PD-1 comprises or consists of the amino acid sequence of SEQ ID NO: 1761.

In another embodiment, the ISVD specifically binding to human PD-1 may have a sequence identity of more than 90%, such as more than 95% or even more than 99%, with SEQ ID NO: 1762, wherein the CDRs are as defined above. In one embodiment, the ISVD specifically binding to PD-1 comprises or consists of the amino acid sequence of SEQ ID NO: 1762.

In another embodiment, the ISVD specifically binding to human PD-1 may have a sequence identity of more than 90%, such as more than 95% or even more than 99%, with SEQ ID NO: 1763, wherein the CDRs are as defined above. In one embodiment, the ISVD specifically binding to PD-1 comprises or consists of the amino acid sequence of SEQ ID NO: 1763.

In another embodiment, the ISVD specifically binding to human PD-1 may have a sequence identity of more than 90%, such as more than 95% or even more than 99%, with SEQ ID NO: 1764, wherein the CDRs are as defined above. In one embodiment, the ISVD specifically binding to PD-1 comprises or consists of the amino acid sequence of SEQ ID NO: 1764.

In another embodiment, the ISVD specifically binding to human PD-1 may have a sequence identity of more than 90%, such as more than 95% or even more than 99%, with SEQ ID NO: 1765, wherein the CDRs are as defined above. In one embodiment, the ISVD specifically binding to PD-1 comprises or consists of the amino acid sequence of SEQ ID NO: 1765.

In another embodiment, the ISVD specifically binding to human PD-1 may have a sequence identity of more than 90%, such as more than 95% or even more than 99%, with SEQ ID NO: 1766, wherein the CDRs are as defined above. In one embodiment, the ISVD specifically binding to PD-1 comprises or consists of the amino acid sequence of SEQ ID NO: 1766.

In another embodiment, the ISVD specifically binding to human PD-1 may have a sequence identity of more than 90%, such as more than 95% or even more than 99%, with SEQ ID NO: 1767, wherein the CDRs are as defined above. In one embodiment, the ISVD specifically binding to PD-1 comprises or consists of the amino acid sequence of SEQ ID NO: 1767.

In another embodiment, the ISVD specifically binding to human PD-1 may have a sequence identity of more than 90%, such as more than 95% or even more than 99%, with SEQ ID NO: 1768, wherein the CDRs are as defined above. In one embodiment, the ISVD specifically binding to PD-1 comprises or consists of the amino acid sequence of SEQ ID NO: 1768.

In another embodiment, the ISVD specifically binding to human PD-1 may have a sequence identity of more than 90%, such as more than 95% or even more than 99%, with SEQ ID NO: 1769, wherein the CDRs are as defined above. In one embodiment, the ISVD specifically binding to PD-1 comprises or consists of the amino acid sequence of SEQ ID NO: 1769.

In another embodiment, the ISVD specifically binding to human PD-1 may have a sequence identity of more than 90%, such as more than 95% or even more than 99%, with SEQ ID NO: 1770, wherein the CDRs are as defined above. In one embodiment, the ISVD specifically binding to PD-1 comprises or consists of the amino acid sequence of SEQ ID NO: 1770.

In another embodiment, the ISVD specifically binding to human PD-1 may have a sequence identity of more than 90%, such as more than 95% or even more than 99%, with SEQ ID NO: 1771, wherein the CDRs are as defined above. In one embodiment, the ISVD specifically binding to PD-1 comprises or consists of the amino acid sequence of SEQ ID NO: 1771.

In another embodiment, the ISVD specifically binding to human PD-1 may have a sequence identity of more than 90%, such as more than 95% or even more than 99%, with SEQ ID NO: 1772, wherein the CDRs are as defined above. In one embodiment, the ISVD specifically binding to PD-1 comprises or consists of the amino acid sequence of SEQ ID NO: 1772.

In another embodiment, the ISVD specifically binding to human PD-1 may have a sequence identity of more than 90%, such as more than 95% or even more than 99%, with SEQ ID NO: 1773, wherein the CDRs are as defined above. In one embodiment, the ISVD specifically binding to PD-1 comprises or consists of the amino acid sequence of SEQ ID NO: 1773.

In another embodiment, the ISVD specifically binding to human PD-1 may have a sequence identity of more than 90%, such as more than 95% or even more than 99%, with SEQ ID NO: 1774, wherein the CDRs are as defined above. In one embodiment, the ISVD specifically binding to PD-1 comprises or consists of the amino acid sequence of SEQ ID NO: 1774.

In another embodiment, the ISVD specifically binding to human PD-1 may have a sequence identity of more than 90%, such as more than 95% or even more than 99%, with SEQ ID NO: 1775, wherein the CDRs are as defined above. In one embodiment, the ISVD specifically binding to PD-1 comprises or consists of the amino acid sequence of SEQ ID NO: 1775.

In another embodiment, the ISVD specifically binding to human PD-1 may have a sequence identity of more than 90%, such as more than 95% or even more than 99%, with SEQ ID NO: 1776, wherein the CDRs are as defined above. In one embodiment, the ISVD specifically binding to PD-1 comprises or consists of the amino acid sequence of SEQ ID NO: 1776.

In another embodiment, the ISVD specifically binding to human PD-1 may have a sequence identity of more than 90%, such as more than 95% or even more than 99%, with SEQ ID NO: 1777, wherein the CDRs are as defined above. In one embodiment, the ISVD specifically binding to PD-1 comprises or consists of the amino acid sequence of SEQ ID NO: 1777.

In another embodiment, the ISVD specifically binding to human PD-1 may have a sequence identity of more than 90%, such as more than 95% or even more than 99%, with SEQ ID NO: 1778, wherein the CDRs are as defined above. In one embodiment, the ISVD specifically binding to PD-1 comprises or consists of the amino acid sequence of SEQ ID NO: 1778.

In another embodiment, the ISVD specifically binding to human PD-1 may have a sequence identity of more than 90%, such as more than 95% or even more than 99%, with SEQ ID NO: 1779, wherein the CDRs are as defined above. In one embodiment, the ISVD specifically binding to PD-1 comprises or consists of the amino acid sequence of SEQ ID NO: 1779.

In another embodiment, the ISVD specifically binding to human PD-1 may have a sequence identity of more than 90%, such as more than 95% or even more than 99%, with SEQ ID NO: 1780, wherein the CDRs are as defined above. In one embodiment, the ISVD specifically binding to PD-1 comprises or consists of the amino acid sequence of SEQ ID NO: 1780.

In another embodiment, the ISVD specifically binding to human PD-1 may have a sequence identity of more than 90%, such as more than 95% or even more than 99%, with SEQ ID NO: 1781, wherein the CDRs are as defined above. In one embodiment, the ISVD specifically binding to PD-1 comprises or consists of the amino acid sequence of SEQ ID NO: 1781.

In another embodiment, the ISVD specifically binding to human PD-1 may have a sequence identity of more than 90%, such as more than 95% or even more than 99%, with SEQ ID NO: 1782, wherein the CDRs are as defined above. In one embodiment, the ISVD specifically binding to PD-1 comprises or consists of the amino acid sequence of SEQ ID NO: 1782.

In another embodiment, the ISVD specifically binding to human PD-1 may have a sequence identity of more than 90%, such as more than 95% or even more than 99%, with SEQ ID NO: 1783, wherein the CDRs are as defined above. In one embodiment, the ISVD specifically binding to PD-1 comprises or consists of the amino acid sequence of SEQ ID NO: 1783.

In another embodiment, the ISVD specifically binding to human PD-1 may have a sequence identity of more than 90%, such as more than 95% or even more than 99%, with SEQ ID NO: 1784, wherein the CDRs are as defined above. In one embodiment, the ISVD specifically binding to PD-1 comprises or consists of the amino acid sequence of SEQ ID NO: 1784.

In another embodiment, the ISVD specifically binding to human PD-1 may have a sequence identity of more than 90%, such as more than 95% or even more than 99%, with SEQ ID NO: 1785, wherein the CDRs are as defined above. In one embodiment, the ISVD specifically binding to PD-1 comprises or consists of the amino acid sequence of SEQ ID NO: 1785.

In another embodiment, the ISVD specifically binding to human PD-1 may have a sequence identity of more than 90%, such as more than 95% or even more than 99%, with SEQ ID NO: 1786, wherein the CDRs are as defined above. In one embodiment, the ISVD specifically binding to PD-1 comprises or consists of the amino acid sequence of SEQ ID NO: 1786.

In another embodiment, the ISVD specifically binding to human PD-1 may have a sequence identity of more than 90%, such as more than 95% or even more than 99%, with SEQ ID NO: 1787, wherein the CDRs are as defined above. In one embodiment, the ISVD specifically binding to PD-1 comprises or consists of the amino acid sequence of SEQ ID NO: 1787.

In another embodiment, the ISVD specifically binding to human PD-1 may have a sequence identity of more than 90%, such as more than 95% or even more than 99%, with SEQ ID NO: 1788, wherein the CDRs are as defined above. In one embodiment, the ISVD specifically binding to PD-1 comprises or consists of the amino acid sequence of SEQ ID NO: 1788.

In another embodiment, the ISVD specifically binding to human PD-1 may have a sequence identity of more than 90%, such as more than 95% or even more than 99%, with SEQ ID NO: 1789, wherein the CDRs are as defined above. In one embodiment, the ISVD specifically binding to PD-1 comprises or consists of the amino acid sequence of SEQ ID NO: 1789.

In another embodiment, the ISVD specifically binding to human PD-1 may have a sequence identity of more than 90%, such as more than 95% or even more than 99%, with SEQ ID NO: 1790, wherein the CDRs are as defined above. In one embodiment, the ISVD specifically binding to PD-1 comprises or consists of the amino acid sequence of SEQ ID NO: 1790.

In another embodiment, the ISVD specifically binding to human PD-1 may have a sequence identity of more than 90%, such as more than 95% or even more than 99%, with SEQ ID NO: 1791, wherein the CDRs are as defined above. In one embodiment, the ISVD specifically binding to PD-1 comprises or consists of the amino acid sequence of SEQ ID NO: 1791.

In another embodiment, the ISVD specifically binding to human PD-1 may have a sequence identity of more than 90%, such as more than 95% or even more than 99%, with SEQ ID NO: 1792, wherein the CDRs are as defined above. In one embodiment, the ISVD specifically binding to PD-1 comprises or consists of the amino acid sequence of SEQ ID NO: 1792.

In another embodiment, the ISVD specifically binding to human PD-1 may have a sequence identity of more than 90%, such as more than 95% or even more than 99%, with SEQ ID NO: 1793, wherein the CDRs are as defined above. In one embodiment, the ISVD specifically binding to PD-1 comprises or consists of the amino acid sequence of SEQ ID NO: 1793.

In another embodiment, the ISVD specifically binding to human PD-1 may have a sequence identity of more than 90%, such as more than 95% or even more than 99%, with SEQ ID NO: 1794, wherein the CDRs are as defined above. In one embodiment, the ISVD specifically binding to PD-1 comprises or consists of the amino acid sequence of SEQ ID NO: 1794.

In another embodiment, the ISVD specifically binding to human PD-1 may have a sequence identity of more than 90%, such as more than 95% or even more than 99%, with SEQ ID NO: 1795, wherein the CDRs are as defined above. In one embodiment, the ISVD specifically binding to PD-1 comprises or consists of the amino acid sequence of SEQ ID NO: 1795.

In another embodiment, the ISVD specifically binding to human PD-1 may have a sequence identity of more than 90%, such as more than 95% or even more than 99%, with SEQ ID NO: 1796, wherein the CDRs are as defined above. In one embodiment, the ISVD specifically binding to PD-1 comprises or consists of the amino acid sequence of SEQ ID NO: 1796.

In another embodiment, the ISVD specifically binding to human PD-1 may have a sequence identity of more than 90%, such as more than 95% or even more than 99%, with SEQ ID NO: 1797, wherein the CDRs are as defined above. In one embodiment, the ISVD specifically binding to PD-1 comprises or consists of the amino acid sequence of SEQ ID NO: 1797.

In another embodiment, the ISVD specifically binding to human PD-1 may have a sequence identity of more than 90%, such as more than 95% or even more than 99%, with SEQ ID NO: 1798, wherein the CDRs are as defined above. In one embodiment, the ISVD specifically binding to PD-1 comprises or consists of the amino acid sequence of SEQ ID NO: 1798.

In another embodiment, the ISVD specifically binding to human PD-1 may have a sequence identity of more than 90%, such as more than 95% or even more than 99%, with SEQ ID NO: 1799, wherein the CDRs are as defined above. In one embodiment, the ISVD specifically binding to PD-1 comprises or consists of the amino acid sequence of SEQ ID NO: 1799.

In another embodiment, the ISVD specifically binding to human PD-1 may have a sequence identity of more than 90%, such as more than 95% or even more than 99%, with SEQ ID NO: 1800, wherein the CDRs are as defined above. In one embodiment, the ISVD specifically binding to PD-1 comprises or consists of the amino acid sequence of SEQ ID NO: 1800.

In another embodiment, the ISVD specifically binding to human PD-1 may have a sequence identity of more than 90%, such as more than 95% or even more than 99%, with SEQ ID NO: 1801, wherein the CDRs are as defined above. In one embodiment, the ISVD specifically binding to PD-1 comprises or consists of the amino acid sequence of SEQ ID NO: 1801.

In another embodiment, the ISVD specifically binding to human PD-1 may have a sequence identity of more than 90%, such as more than 95% or even more than 99%, with SEQ ID NO: 1802, wherein the CDRs are as defined above. In one embodiment, the ISVD specifically binding to PD-1 comprises or consists of the amino acid sequence of SEQ ID NO: 1802.

In another embodiment, the ISVD specifically binding to human PD-1 may have a sequence identity of more than 90%, such as more than 95% or even more than 99%, with SEQ ID NO: 1803, wherein the CDRs are as defined above. In one embodiment, the ISVD specifically binding to PD-1 comprises or consists of the amino acid sequence of SEQ ID NO: 1803.

In another embodiment, the ISVD specifically binding to human PD-1 may have a sequence identity of more than 90%, such as more than 95% or even more than 99%, with SEQ ID NO: 1804, wherein the CDRs are as defined above. In one embodiment, the ISVD specifically binding to PD-1 comprises or consists of the amino acid sequence of SEQ ID NO: 1804.

In another embodiment, the ISVD specifically binding to human PD-1 may have a sequence identity of more than 90%, such as more than 95% or even more than 99%, with SEQ ID NO: 1805, wherein the CDRs are as defined above. In one embodiment, the ISVD specifically binding to PD-1 comprises or consists of the amino acid sequence of SEQ ID NO: 1805.

In another embodiment, the ISVD specifically binding to human PD-1 may have a sequence identity of more than 90%, such as more than 95% or even more than 99%, with SEQ ID NO: 1806, wherein the CDRs are as defined above. In one embodiment, the ISVD specifically binding to PD-1 comprises or consists of the amino acid sequence of SEQ ID NO: 1806.

In another embodiment, the ISVD specifically binding to human PD-1 may have a sequence identity of more than 90%, such as more than 95% or even more than 99%, with SEQ ID NO: 1807, wherein the CDRs are as defined above. In one embodiment, the ISVD specifically binding to PD-1 comprises or consists of the amino acid sequence of SEQ ID NO: 1807.

In another embodiment, the ISVD specifically binding to human PD-1 may have a sequence identity of more than 90%, such as more than 95% or even more than 99%, with SEQ ID NO: 1808, wherein the CDRs are as defined above. In one embodiment, the ISVD specifically binding to PD-1 comprises or consists of the amino acid sequence of SEQ ID NO: 1808.

In another embodiment, the ISVD specifically binding to human PD-1 may have a sequence identity of more than 90%, such as more than 95% or even more than 99%, with SEQ ID NO: 1809, wherein the CDRs are as defined above. In one embodiment, the ISVD specifically binding to PD-1 comprises or consists of the amino acid sequence of SEQ ID NO: 1809.

In another embodiment, the ISVD specifically binding to human PD-1 may have a sequence identity of more than 90%, such as more than 95% or even more than 99%, with SEQ ID NO: 1810, wherein the CDRs are as defined above. In one embodiment, the ISVD specifically binding to PD-1 comprises or consists of the amino acid sequence of SEQ ID NO: 1810.

In another embodiment, the ISVD specifically binding to human PD-1 may have a sequence identity of more than 90%, such as more than 95% or even more than 99%, with SEQ ID NO: 1811, wherein the CDRs are as defined above. In one embodiment, the ISVD specifically binding to PD-1 comprises or consists of the amino acid sequence of SEQ ID NO: 1811.

In another embodiment, the ISVD specifically binding to human PD-1 may have a sequence identity of more than 90%, such as more than 95% or even more than 99%, with SEQ ID NO: 1812, wherein the CDRs are as defined above. In one embodiment, the ISVD specifically binding to PD-1 comprises or consists of the amino acid sequence of SEQ ID NO: 1812.

In another embodiment, the ISVD specifically binding to human PD-1 may have a sequence identity of more than 90%, such as more than 95% or even more than 99%, with SEQ ID NO: 1813, wherein the CDRs are as defined above. In one embodiment, the ISVD specifically binding to PD-1 comprises or consists of the amino acid sequence of SEQ ID NO: 1813.

In another embodiment, the ISVD specifically binding to human PD-1 may have a sequence identity of more than 90%, such as more than 95% or even more than 99%, with SEQ ID NO: 1814, wherein the CDRs are as defined above. In one embodiment, the ISVD specifically binding to PD-1 comprises or consists of the amino acid sequence of SEQ ID NO: 1814.

In another embodiment, the ISVD specifically binding to human PD-1 may have a sequence identity of more than 90%, such as more than 95% or even more than 99%, with SEQ ID NO: 1815, wherein the CDRs are as defined above. In one embodiment, the ISVD specifically binding to PD-1 comprises or consists of the amino acid sequence of SEQ ID NO: 1815.

In another embodiment, the ISVD specifically binding to human PD-1 may have a sequence identity of more than 90%, such as more than 95% or even more than 99%, with SEQ ID NO: 1816, wherein the CDRs are as defined above. In one embodiment, the ISVD specifically binding to PD-1 comprises or consists of the amino acid sequence of SEQ ID NO: 1816.

In another embodiment, the ISVD specifically binding to human PD-1 may have a sequence identity of more than 90%, such as more than 95% or even more than 99%, with SEQ ID NO: 1817, wherein the CDRs are as defined above. In one embodiment, the ISVD specifically binding to PD-1 comprises or consists of the amino acid sequence of SEQ ID NO: 1817.

In another embodiment, the ISVD specifically binding to human PD-1 may have a sequence identity of more than 90%, such as more than 95% or even more than 99%, with SEQ ID NO: 1818, wherein the CDRs are as defined above. In one embodiment, the ISVD specifically binding to PD-1 comprises or consists of the amino acid sequence of SEQ ID NO: 1818.

In another embodiment, the ISVD specifically binding to human PD-1 may have a sequence identity of more than 90%, such as more than 95% or even more than 99%, with SEQ ID NO: 1819, wherein the CDRs are as defined above. In one embodiment, the ISVD specifically binding to PD-1 comprises or consists of the amino acid sequence of SEQ ID NO: 1819.

In another embodiment, the ISVD specifically binding to human PD-1 may have a sequence identity of more than 90%, such as more than 95% or even more than 99%, with SEQ ID NO: 1820, wherein the CDRs are as defined above. In one embodiment, the ISVD specifically binding to PD-1 comprises or consists of the amino acid sequence of SEQ ID NO: 1820.

In another embodiment, the ISVD specifically binding to human PD-1 may have a sequence identity of more than 90%, such as more than 95% or even more than 99%, with SEQ ID NO: 1821, wherein the CDRs are as defined above. In one embodiment, the ISVD specifically binding to PD-1 comprises or consists of the amino acid sequence of SEQ ID NO: 1821.

In another embodiment, the ISVD specifically binding to human PD-1 may have a sequence identity of more than 90%, such as more than 95% or even more than 99%, with SEQ ID NO: 1822, wherein the CDRs are as defined above. In one embodiment, the ISVD specifically binding to PD-1 comprises or consists of the amino acid sequence of SEQ ID NO: 1822.

In another embodiment, the ISVD specifically binding to human PD-1 may have a sequence identity of more than 90%, such as more than 95% or even more than 99%, with SEQ ID NO: 1823, wherein the CDRs are as defined above. In one embodiment, the ISVD specifically binding to PD-1 comprises or consists of the amino acid sequence of SEQ ID NO: 1823.

In another embodiment, the ISVD specifically binding to human PD-1 may have a sequence identity of more than 90%, such as more than 95% or even more than 99%, with SEQ ID NO: 1824, wherein the CDRs are as defined above. In one embodiment, the ISVD specifically binding to PD-1 comprises or consists of the amino acid sequence of SEQ ID NO: 1824.

In another embodiment, the ISVD specifically binding to human PD-1 may have a sequence identity of more than 90%, such as more than 95% or even more than 99%, with SEQ ID NO: 1825, wherein the CDRs are as defined above. In one embodiment, the ISVD specifically binding to PD-1 comprises or consists of the amino acid sequence of SEQ ID NO: 1825.

In another embodiment, the ISVD specifically binding to human PD-1 may have a sequence identity of more than 90%, such as more than 95% or even more than 99%, with SEQ ID NO: 1826, wherein the CDRs are as defined above. In one embodiment, the ISVD specifically binding to PD-1 comprises or consists of the amino acid sequence of SEQ ID NO: 1826.

In another embodiment, the ISVD specifically binding to human PD-1 may have a sequence identity of more than 90%, such as more than 95% or even more than 99%, with SEQ ID NO: 1827, wherein the CDRs are as defined above. In one embodiment, the ISVD specifically binding to PD-1 comprises or consists of the amino acid sequence of SEQ ID NO: 1827.

In another embodiment, the ISVD specifically binding to human PD-1 may have a sequence identity of more than 90%, such as more than 95% or even more than 99%, with SEQ ID NO: 1828, wherein the CDRs are as defined above. In one embodiment, the ISVD specifically binding to PD-1 comprises or consists of the amino acid sequence of SEQ ID NO: 1828.

In another embodiment, the ISVD specifically binding to human PD-1 may have a sequence identity of more than 90%, such as more than 95% or even more than 99%, with SEQ ID NO: 1829, wherein the CDRs are as defined above. In one embodiment, the ISVD specifically binding to PD-1 comprises or consists of the amino acid sequence of SEQ ID NO: 1829.

In another embodiment, the ISVD specifically binding to human PD-1 may have a sequence identity of more than 90%, such as more than 95% or even more than 99%, with SEQ ID NO: 1830, wherein the CDRs are as defined above. In one embodiment, the ISVD specifically binding to PD-1 comprises or consists of the amino acid sequence of SEQ ID NO: 1830.

In another embodiment, the ISVD specifically binding to human PD-1 may have a sequence identity of more than 90%, such as more than 95% or even more than 99%, with SEQ ID NO: 1831, wherein the CDRs are as defined above. In one embodiment, the ISVD specifically binding to PD-1 comprises or consists of the amino acid sequence of SEQ ID NO: 1831.

In another embodiment, the ISVD specifically binding to human PD-1 may have a sequence identity of more than 90%, such as more than 95% or even more than 99%, with SEQ ID NO: 1832, wherein the CDRs are as defined above. In one embodiment, the ISVD specifically binding to PD-1 comprises or consists of the amino acid sequence of SEQ ID NO: 1832.

In another embodiment, the ISVD specifically binding to human PD-1 may have a sequence identity of more than 90%, such as more than 95% or even more than 99%, with SEQ ID NO: 1833, wherein the CDRs are as defined above. In one embodiment, the ISVD specifically binding to PD-1 comprises or consists of the amino acid sequence of SEQ ID NO: 1833.

In another embodiment, the ISVD specifically binding to human PD-1 may have a sequence identity of more than 90%, such as more than 95% or even more than 99%, with SEQ ID NO: 1834, wherein the CDRs are as defined above. In one embodiment, the ISVD specifically binding to PD-1 comprises or consists of the amino acid sequence of SEQ ID NO: 1834.

In another embodiment, the ISVD specifically binding to human PD-1 may have a sequence identity of more than 90%, such as more than 95% or even more than 99%, with SEQ ID NO: 1835, wherein the CDRs are as defined above. In one embodiment, the ISVD specifically binding to PD-1 comprises or consists of the amino acid sequence of SEQ ID NO: 1835.

In another embodiment, the ISVD specifically binding to human PD-1 may have a sequence identity of more than 90%, such as more than 95% or even more than 99%, with SEQ ID NO: 1836, wherein the CDRs are as defined above. In one embodiment, the ISVD specifically binding to PD-1 comprises or consists of the amino acid sequence of SEQ ID NO: 1836.

In another embodiment, the ISVD specifically binding to human PD-1 may have a sequence identity of more than 90%, such as more than 95% or even more than 99%, with SEQ ID NO: 1837, wherein the CDRs are as defined above. In one embodiment, the ISVD specifically binding to PD-1 comprises or consists of the amino acid sequence of SEQ ID NO: 1837.

In another embodiment, the ISVD specifically binding to human PD-1 may have a sequence identity of more than 90%, such as more than 95% or even more than 99%, with SEQ ID NO: 1838, wherein the CDRs are as defined above. In one embodiment, the ISVD specifically binding to PD-1 comprises or consists of the amino acid sequence of SEQ ID NO: 1838.

In another embodiment, the ISVD specifically binding to human PD-1 may have a sequence identity of more than 90%, such as more than 95% or even more than 99%, with SEQ ID NO: 1839, wherein the CDRs are as defined above. In one embodiment, the ISVD specifically binding to PD-1 comprises or consists of the amino acid sequence of SEQ ID NO: 1839.

In another embodiment, the ISVD specifically binding to human PD-1 may have a sequence identity of more than 90%, such as more than 95% or even more than 99%, with SEQ ID NO: 1840, wherein the CDRs are as defined above. In one embodiment, the ISVD specifically binding to PD-1 comprises or consists of the amino acid sequence of SEQ ID NO: 1840.

In another embodiment, the ISVD specifically binding to human PD-1 may have a sequence identity of more than 90%, such as more than 95% or even more than 99%, with SEQ ID NO: 1841, wherein the CDRs are as defined above. In one embodiment, the ISVD specifically binding to PD-1 comprises or consists of the amino acid sequence of SEQ ID NO: 1841.

In another embodiment, the ISVD specifically binding to human PD-1 may have a sequence identity of more than 90%, such as more than 95% or even more than 99%, with SEQ ID NO: 1842, wherein the CDRs are as defined above. In one embodiment, the ISVD specifically binding to PD-1 comprises or consists of the amino acid sequence of SEQ ID NO: 1842.

In another embodiment, the ISVD specifically binding to human PD-1 may have a sequence identity of more than 90%, such as more than 95% or even more than 99%, with SEQ ID NO: 1843, wherein the CDRs are as defined above. In one embodiment, the ISVD specifically binding to PD-1 comprises or consists of the amino acid sequence of SEQ ID NO: 1843.

In another embodiment, the ISVD specifically binding to human PD-1 may have a sequence identity of more than 90%, such as more than 95% or even more than 99%, with SEQ ID NO: 1844, wherein the CDRs are as defined above. In one embodiment, the ISVD specifically binding to PD-1 comprises or consists of the amino acid sequence of SEQ ID NO: 1844.

In another embodiment, the ISVD specifically binding to human PD-1 may have a sequence identity of more than 90%, such as more than 95% or even more than 99%, with SEQ ID NO: 1845, wherein the CDRs are as defined above. In one embodiment, the ISVD specifically binding to PD-1 comprises or consists of the amino acid sequence of SEQ ID NO: 1845.

In another embodiment, the ISVD specifically binding to human PD-1 may have a sequence identity of more than 90%, such as more than 95% or even more than 99%, with SEQ ID NO: 1846, wherein the CDRs are as defined above. In one embodiment, the ISVD specifically binding to PD-1 comprises or consists of the amino acid sequence of SEQ ID NO: 1846.

In another embodiment, the ISVD specifically binding to human PD-1 may have a sequence identity of more than 90%, such as more than 95% or even more than 99%, with SEQ ID NO: 1847, wherein the CDRs are as defined above. In one embodiment, the ISVD specifically binding to PD-1 comprises or consists of the amino acid sequence of SEQ ID NO: 1847.

In another embodiment, the ISVD specifically binding to human PD-1 may have a sequence identity of more than 90%, such as more than 95% or even more than 99%, with SEQ ID NO: 1848, wherein the CDRs are as defined above. In one embodiment, the ISVD specifically binding to PD-1 comprises or consists of the amino acid sequence of SEQ ID NO: 1848.

In another embodiment, the ISVD specifically binding to human PD-1 may have a sequence identity of more than 90%, such as more than 95% or even more than 99%, with SEQ ID NO: 1849, wherein the CDRs are as defined above. In one embodiment, the ISVD specifically binding to PD-1 comprises or consists of the amino acid sequence of SEQ ID NO: 1849.

In another embodiment, the ISVD specifically binding to human PD-1 may have a sequence identity of more than 90%, such as more than 95% or even more than 99%, with SEQ ID NO: 1850, wherein the CDRs are as defined above. In one embodiment, the ISVD specifically binding to PD-1 comprises or consists of the amino acid sequence of SEQ ID NO: 1850.

In another embodiment, the ISVD specifically binding to human PD-1 may have a sequence identity of more than 90%, such as more than 95% or even more than 99%, with SEQ ID NO: 1851, wherein the CDRs are as defined above. In one embodiment, the ISVD specifically binding to PD-1 comprises or consists of the amino acid sequence of SEQ ID NO: 1851.

In another embodiment, the ISVD specifically binding to human PD-1 may have a sequence identity of more than 90%, such as more than 95% or even more than 99%, with SEQ ID NO: 1852, wherein the CDRs are as defined above. In one embodiment, the ISVD specifically binding to PD-1 comprises or consists of the amino acid sequence of SEQ ID NO: 1852.

In another embodiment, the ISVD specifically binding to human PD-1 may have a sequence identity of more than 90%, such as more than 95% or even more than 99%, with SEQ ID NO: 1853, wherein the CDRs are as defined above. In one embodiment, the ISVD specifically binding to PD-1 comprises or consists of the amino acid sequence of SEQ ID NO: 1853.

In another embodiment, the ISVD specifically binding to human PD-1 may have a sequence identity of more than 90%, such as more than 95% or even more than 99%, with SEQ ID NO: 1854, wherein the CDRs are as defined above. In one embodiment, the ISVD specifically binding to PD-1 comprises or consists of the amino acid sequence of SEQ ID NO: 1854.

In another embodiment, the ISVD specifically binding to human PD-1 may have a sequence identity of more than 90%, such as more than 95% or even more than 99%, with SEQ ID NO: 1855, wherein the CDRs are as defined above. In one embodiment, the ISVD specifically binding to PD-1 comprises or consists of the amino acid sequence of SEQ ID NO: 1855.

In another embodiment, the ISVD specifically binding to human PD-1 may have a sequence identity of more than 90%, such as more than 95% or even more than 99%, with SEQ ID NO: 1856, wherein the CDRs are as defined above. In one embodiment, the ISVD specifically binding to PD-1 comprises or consists of the amino acid sequence of SEQ ID NO: 1856.

In another embodiment, the ISVD specifically binding to human PD-1 may have a sequence identity of more than 90%, such as more than 95% or even more than 99%, with SEQ ID NO: 1857, wherein the CDRs are as defined above. In one embodiment, the ISVD specifically binding to PD-1 comprises or consists of the amino acid sequence of SEQ ID NO: 1857.

In another embodiment, the ISVD specifically binding to human PD-1 may have a sequence identity of more than 90%, such as more than 95% or even more than 99%, with SEQ ID NO: 1858, wherein the CDRs are as defined above. In one embodiment, the ISVD specifically binding to PD-1 comprises or consists of the amino acid sequence of SEQ ID NO: 1858.

In another embodiment, the ISVD specifically binding to human PD-1 may have a sequence identity of more than 90%, such as more than 95% or even more than 99%, with SEQ ID NO: 1859, wherein the CDRs are as defined above. In one embodiment, the ISVD specifically binding to PD-1 comprises or consists of the amino acid sequence of SEQ ID NO: 1859.

In another embodiment, the ISVD specifically binding to human PD-1 may have a sequence identity of more than 90%, such as more than 95% or even more than 99%, with SEQ ID NO: 1860, wherein the CDRs are as defined above. In one embodiment, the ISVD specifically binding to PD-1 comprises or consists of the amino acid sequence of SEQ ID NO: 1860.

In another embodiment, the ISVD specifically binding to human PD-1 may have a sequence identity of more than 90%, such as more than 95% or even more than 99%, with SEQ ID NO: 1861, wherein the CDRs are as defined above. In one embodiment, the ISVD specifically binding to PD-1 comprises or consists of the amino acid sequence of SEQ ID NO: 1861.

In another embodiment, the ISVD specifically binding to human PD-1 may have a sequence identity of more than 90%, such as more than 95% or even more than 99%, with SEQ ID NO: 1862, wherein the CDRs are as defined above. In one embodiment, the ISVD specifically binding to PD-1 comprises or consists of the amino acid sequence of SEQ ID NO: 1862.

In another embodiment, the ISVD specifically binding to human PD-1 may have a sequence identity of more than 90%, such as more than 95% or even more than 99%, with SEQ ID NO: 1863, wherein the CDRs are as defined above. In one embodiment, the ISVD specifically binding to PD-1 comprises or consists of the amino acid sequence of SEQ ID NO: 1863.

In another embodiment, the ISVD specifically binding to human PD-1 may have a sequence identity of more than 90%, such as more than 95% or even more than 99%, with SEQ ID NO: 1864, wherein the CDRs are as defined above. In one embodiment, the ISVD specifically binding to PD-1 comprises or consists of the amino acid sequence of SEQ ID NO: 1864.

In another embodiment, the ISVD specifically binding to human PD-1 may have a sequence identity of more than 90%, such as more than 95% or even more than 99%, with SEQ ID NO: 1865, wherein the CDRs are as defined above. In one embodiment, the ISVD specifically binding to PD-1 comprises or consists of the amino acid sequence of SEQ ID NO: 1865.

In another embodiment, the ISVD specifically binding to human PD-1 may have a sequence identity of more than 90%, such as more than 95% or even more than 99%, with SEQ ID NO: 1866, wherein the CDRs are as defined above. In one embodiment, the ISVD specifically binding to PD-1 comprises or consists of the amino acid sequence of SEQ ID NO: 1866.

In another embodiment, the ISVD specifically binding to human PD-1 may have a sequence identity of more than 90%, such as more than 95% or even more than 99%, with SEQ ID NO: 1867, wherein the CDRs are as defined above. In one embodiment, the ISVD specifically binding to PD-1 comprises or consists of the amino acid sequence of SEQ ID NO: 1867.

In another embodiment, the ISVD specifically binding to human PD-1 may have a sequence identity of more than 90%, such as more than 95% or even more than 99%, with SEQ ID NO: 1868, wherein the CDRs are as defined above. In one embodiment, the ISVD specifically binding to PD-1 comprises or consists of the amino acid sequence of SEQ ID NO: 1868.

In another embodiment, the ISVD specifically binding to human PD-1 may have a sequence identity of more than 90%, such as more than 95% or even more than 99%, with SEQ ID NO: 1869, wherein the CDRs are as defined above. In one embodiment, the ISVD specifically binding to PD-1 comprises or consists of the amino acid sequence of SEQ ID NO: 1869.

In another embodiment, the ISVD specifically binding to human PD-1 may have a sequence identity of more than 90%, such as more than 95% or even more than 99%, with SEQ ID NO: 1870, wherein the CDRs are as defined above. In one embodiment, the ISVD specifically binding to PD-1 comprises or consists of the amino acid sequence of SEQ ID NO: 1870.

In another embodiment, the ISVD specifically binding to human PD-1 may have a sequence identity of more than 90%, such as more than 95% or even more than 99%, with SEQ ID NO: 1871, wherein the CDRs are as defined above. In one embodiment, the ISVD specifically binding to PD-1 comprises or consists of the amino acid sequence of SEQ ID NO: 1871.

Specific examples of ISVDs specifically binding to PD-1 are ISVDs that comprises 4 framework regions (FR1 to FR4, respectively) and three complementarity determining regions (CDR1 to CDR3, respectively), wherein:
CDR1 (AbM numbering) consists of an amino acid sequence selected from:
   a) the amino acid sequence of GDTFSENDIG (SEQ ID NO: 1957);
   b) amino acid sequences that have at least 80% amino acid identity with the amino acid sequence of GDTFSENDIG (SEQ ID NO: 1957);
   c) amino acid sequences that have 3, 2, or 1 amino acid difference with the amino acid sequences of GDTFSENDIG (SEQ ID NO: 1957);
and
CDR2 (AbM numbering) consists of an amino acid sequence selected from:
   a) the amino acid sequence of AIVWPGGETY (SEQ ID NO: 2127);
   b) amino acid sequences that have at least 80% amino acid identity with the amino acid sequence of AIVWPGGETY (SEQ ID NO: 2127);
   c) amino acid sequences that have 3, 2, or 1 amino acid difference with the amino acid sequence of AIVWPGGETY (SEQ ID NO: 2127);
and
CDR3 (AbM numbering) consists of an amino acid sequence selected from:
   a) the amino acid sequence of KIFSKTGTYSASWEYDF (SEQ ID NO: 2337);
   b) amino acid sequences that have at least 80% amino acid identity with the amino acid sequence KIFSKTGTYSASWEYDF (SEQ ID NO: 2337);
   c) amino acid sequences that have 3, 2, or 1 amino acid difference with the amino acid sequence of KIFSKTGTYSASWEYDF (SEQ ID NO: 2337).

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NO: 1957, SEQ ID NO: 2127 and/or SEQ ID NO: 2337.

In one embodiment, the ISVD comprises CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 793, 1505, 1092, 1085, 1078, 1486, or 1083.

In one embodiment, the ISVD comprises a CDR1 (AbM numbering) that is the amino acid sequence of SEQ ID NO: 1957, a CDR2 (AbM numbering) that is the amino acid sequence of SEQ ID NO: 2127 and a CDR3 (AbM numbering) that is the amino acid sequence of SEQ ID NO: 2337.

In some embodimenst, the ISVD additionally comprises D1 and/or V2 (Kabat numbering).

Specific examples of such ISVDs that specifically bind to PD-1 have one or more, or all, framework regions as indicated for A0294041A11 and sequence optimized variants thereof in Table A-3 (in addition to the CDRs as defined above). In one embodiment, the ISVD comprises or consists of the full amino acid sequence of A0294041A11 (SEQ ID NO: 793), A029400157 (SEQ ID NO: 1505), A029405240 (SEQ ID NO: 1092), A029405247 (SEQ ID NO: 1085), A029405255 (SEQ ID NO: 1078), or A029405259 (SEQ ID NO: 1083) (see Table A-3 and A-8).

The SEQ ID NOs for the CDR sequences referred to above are based on the CDR definition according to the AbM definition (see Table A-8). It is noted that the SEQ ID NOs for the CDR sequences defined according to the Kabat definition, Chothia definition or IMGT definition can likewise be used. Accordingly, the ISVDs provided by the present technology, specifically binding to PD-1 as described above using the AbM definition, can be also described using the Kabat definition, Chothia definition or IMGT definition

In one embodiment, the ISVD comprises CDRs (Kabat definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 714-1871.

In one embodiment, the ISVD comprises CDRs (Kabat definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 793, 1505, 1092, 1085, 1078, 1486, and 1083.

In one embodiment, the ISVD comprises CDRs (Chothia definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 714-1871, preferably selected from SEQ ID NOs: 793, 1505, 1092, 1085, 1078, 1486, and 1083.

In one embodiment, the ISVD comprises CDRs (IMGT definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 714-1871, preferably selected from SEQ ID NOs: 793, 1505, 1092, 1085, 1078, 1486, and 1083.

In another embodiment, when such an ISVD specifically binding to PD-1 has 3, 2 or 1 amino acid difference in at least one CDR relative to a corresponding reference CDR sequence (above), the ISVD has at least (essentially) the same binding affinity to human PD-1 compared to one of the VHHs with SEQ ID NOs: 793, 1505, 1092, 1085, 1078, 1486, or 1083, wherein the binding affinity is measured using the same method, such as e.g. SPR.

In another embodiment, when such an ISVD specifically binding to PD-1 has 3, 2 or 1 amino acid difference in at least one CDR relative to a corresponding reference CDR sequence (above), the ISVD has at least (essentially) the same binding affinity to cyno PD-1 compared to one of the VHHs with SEQ ID NOs: 793, 1505, 1092, 1085, 1078, 1486, or 1083, wherein the binding affinity is measured using the same method, such as e.g. SPR.

In another embodiment, when such an ISVD specifically binding to PD-1 has 3, 2 or 1 amino acid difference in at least one CDR relative to a corresponding reference CDR sequence (above), the ISVD still specifically binds to human PD-1 and cyno PD-1 with less than 10-fold difference in affinity (KD).

In another embodiment, when such an ISVD specifically binding to PD-1 has 3, 2 or 1 amino acid difference in at least one CDR relative to a corresponding reference CDR sequence (above), the ISVD still specifically binds to human and cyno PD-1 and does not bind to other members of the CD28 receptor family.

In another embodiment, when such an ISVD specifically binding to PD-1 has 3, 2 or 1 amino acid difference in at least one CDR relative to a corresponding reference CDR sequence (above), the ISVD still specifically binds to human and cyno PD-1 and does not bind to CD28, CTLA-4, ICOS, and BTLA.

In another embodiment, when such an ISVD specifically binding to PD-1 has 3, 2 or 1 amino acid difference in at least one CDR relative to a corresponding reference CDR sequence (above), the ISVD blocks the interaction of human PD-1 with human PD-L1 with at least (essentially) the same potency compared to one of the VHHs with SEQ ID NOs: 793, 1505, 1092, 1085, 1078, 1486, or 1083, wherein the potency is measured, e.g., in FACS competition assay on CHO cells expressing human PD-1, with hPD-L1 as competitor.

In another embodiment, when such an ISVD specifically binding to PD-1 has 3, 2 or 1 amino acid difference in at least one CDR relative to a corresponding reference CDR sequence (above), the ISVD blocks the interaction of cyno PD-1 with cyno PD-L1 with at least (essentially) the same potency compared to one of the VHHs with SEQ ID NOs: 793, 1505, 1092, 1085, 1078, 1486, or 1083, wherein the potency is measured, e.g., in FACS competition assay on CHO cells expressing cyno PD-1, with hPD-L1 as competitor.

In another embodiment, when such an ISVD specifically binding to PD-1 has 3, 2 or 1 amino acid difference in at least one CDR relative to a corresponding reference CDR sequence (above), the ISVD blocks the interaction of hPD-1 with PD-L1 with (essentially) the same % inhibition or more compared to one of the VHHs with SEQ ID NOs: 793, 1505, 1092, 1085, 1078, 1486, or 1083, wherein the % inhibition is determined, e.g., in a FACS competition assay.

In another embodiment, when such an ISVD specifically binding to PD-1 has 3, 2 or 1 amino acid difference in at least one CDR relative to a corresponding reference CDR sequence (above), the ISVD has at least (essentially) the same potency for T-cell activation compared to one of the VHHs with SEQ ID NOs: 793, 1505, 1092, 1085, 1078, 1486, or 1083, wherein the potency is monitored, e.g., via IFN-gamma production in an autologous Tetanus Toxoid recall assay (TT assay), e.g. the TT assay as described herein.

In another embodiment, when such an ISVD specifically binding to PD-1 has 3, 2 or 1 amino acid difference in at least one CDR relative to a corresponding reference CDR sequence (above), the ISVD has at least (essentially) the same efficacy for T-cell activation compared to one of the VHHs with SEQ ID NOs: 793, 1505, 1092, 1085, 1078, 1486, or 1083, wherein the efficacy is monitored, e.g., via IFN-gamma production in an autologous Tetanus Toxoid recall assay (TT assay), e.g. the TT assay as described herein

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same binding affinity to human PD-1 compared to one of the VHHs with SEQ ID NOs: 793, 1505, 1092, 1085, 1078, 1486, or 1083, wherein the binding affinity is measured using the same method, such as e.g. SPR.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same binding affinity to cyno PD-1 compared to one of the VHHs with SEQ ID NOs: 793, 1505, 1092, 1085, 1078, 1486, or 1083, wherein the binding affinity is measured using the same method, such as e.g. SPR.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human PD-1 and cyno PD-1 with less than 10-fold difference in affinity (KD).

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human and cyno PD-1 and does not bind to other members of the CD28 receptor family.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human and cyno PD-1 and does not bind to CD28, CTLA-4, ICOS, and BTLA.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of human PD-1 with human PD-L1 with at least (essentially) the same potency compared to one of the VHHs with SEQ ID NOs: 793, 1505, 1092, 1085, 1078, 1486, or 1083, wherein the potency is measured, e.g., in FACS competition assay on CHO cells expressing human PD-1, with hPD-L1 as competitor.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of cyno PD-1 with cyno PD-L1 with at least (essentially) the same potency compared to one of the VHHs with SEQ ID NOs: 793, 1505, 1092, 1085, 1078, 1486, or 1083, wherein the potency is measured, e.g., in FACS competition assay on CHO cells expressing cyno PD-1, with hPD-L1 as competitor.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of hPD-1 with (h) PD-L1 with (essentially) the same % inhibition or more compared to one of the VHHs with SEQ ID NOs: 793, 1505, 1092, 1085, 1078, 1486, or 1083, wherein the % inhibition is determined, e.g., in FACS competition assay.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same potency for T-cell activation compared to one of the VHHs with SEQ ID NOs: 793, 1505, 1092, 1085, 1078, 1486, or 1083, wherein the potency is monitored, e.g., via IFN-gamma production in an autologous Tetanus Toxoid recall assay (TT assay), e.g. the TT assay as described herein.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same efficacy for T-cell activation compared to one of the VHHs with SEQ ID NOs: 793, 1505, 1092, 1085, 1078, 1486, or 1083, wherein the efficacy is monitored, e.g., via IFN-gamma production in an autologous Tetanus Toxoid recall assay (TT assay), e.g. the TT assay as described herein.

In another embodiment, when the ISVD specifically binding to human PD-1 has a sequence identity of more than 90%, such as more than 95% or more than 99%, with one of the VHHs with SEQ ID NOs: 793, 1505, 1092, 1085, 1078, or 1083, the ISVD has at least (essentially) the same binding affinity to human PD-1 compared to one of the VHHs with SEQ ID NOs: 793, 1505, 1092, 1085, 1078, 1486, or 1083, wherein the binding affinity is measured using the same method, such as e.g. SPR.

In another embodiment, when the ISVD specifically binding to human PD-1 has a sequence identity of more than 90%, such as more than 95% or more than 99%, with one of the VHHs with SEQ ID NOs: 793, 1505, 1092, 1085, 1078, or 1083, the ISVD has at least (essentially) the same binding affinity to cyno PD-1 compared to one of the VHHs with SEQ ID NOs: 793, 1505, 1092, 1085, 1078, 1486, or 1083, wherein the binding affinity is measured using the same method, such as e.g. SPR.

In another embodiment, when the ISVD specifically binding to human PD-1 has a sequence identity of more than 90%, such as more than 95% or more than 99%, with one of the VHHs with SEQ ID NOS: 793, 1505, 1092, 1085, 1078, 1486, or 1083, the ISVD still specifically binds to human PD-1 and cyno PD-1 with less than 10-fold difference in affinity (KD).

In another embodiment, when the ISVD specifically binding to human PD-1 has a sequence identity of more than 90%, such as more than 95% or more than 99%, with one of the VHHs with SEQ ID NOs: 793, 1505, 1092, 1085, 1078, 1486, or 1083, the ISVD still specifically binds to human and cyno PD-1 and does not bind to other members of the CD28 receptor family.

In another embodiment, when the ISVD specifically binding to human PD-1 has a sequence identity of more than 90%, such as more than 95% or more than 99%, with one of the VHHs with SEQ ID NOs: 793, 1505, 1092, 1085, 1078, 1486, or 1083, the ISVD still specifically binds to human and cyno PD-1 and does not bind to CD28, CTLA-4, ICOS, and BTLA.

In another embodiment, when the ISVD specifically binding to human PD-1 has a sequence identity of more than 90%, such as more than 95% or more than 99%, with one of the VHHs with SEQ ID NOS: 793, 1505, 1092, 1085, 1078, or 1083, the ISVD blocks the interaction of human PD-1 with human PD-L1 with at least (essentially) the same potency compared to one of the VHHs with SEQ ID NOs: 793, 1505, 1092, 1085, 1078, 1486, or 1083, wherein the potency is measured in wherein the potency is measured, e.g., in a FACS competition assay on CHO cells expressing human PD-1, with hPD-L1 as competitor.

In another embodiment, when the ISVD specifically binding to human PD-1 has a sequence identity of more than 90%, such as more than 95% or more than 99%, with one of the VHHs with SEQ ID NOS: 793, 1505, 1092, 1085, 1078, or 1083, the ISVD blocks the interaction of cyno PD-1 with cyno PD-L1 with at least (essentially) the same potency compared to one of the VHHs with SEQ ID NOs: 793, 1505, 1092, 1085, 1078, 1486, or 1083, wherein the potency is measured, e.g., in FACS competition assay on CHO cells expressing cyno PD-1, with hPD-L1 as competitor.

In another embodiment, when the ISVD specifically binding to human PD-1 has a sequence identity of more than 90%, such as more than 95% or more than 99%, with one of the VHHs with SEQ ID NOS: 793, 1505, 1092, 1085, 1078, or 1083, the ISVD blocks the interaction of hPD-1 with (h) PD-L1 with (essentially) the same % inhibition or more compared to one of the VHHs with SEQ ID NOs: 793, 1505, 1092, 1085, 1078, or 1083, wherein the % inhibition is determined, e.g., in a FACS competition assay.

In another embodiment, when the ISVD specifically binding to human PD-1 has a sequence identity of more than 90%, such as more than 95% or more than 99%, with one of the VHHs with SEQ ID NOs: 793, 1505, 1092, 1085, 1078, 1486, or 1083, the ISVD has at least (essentially) half of the potency, preferably at least (essentially) the same potency for T-cell activation compared to one of the VHHs with SEQ ID NOs: 793, 1505, 1092, 1085, 1078, 1486, or 1083, wherein the potency is monitored, e.g., via IFN-gamma production in an autologous Tetanus Toxoid recall assay (TT assay), e.g. the TT assay as described herein.

In another embodiment, when the ISVD specifically binding to human PD-1 has a sequence identity of more than 90%, such as more than 95% or more than 99%, with one of the VHHs with SEQ ID NOs: 793, 1505, 1092, 1085, 1078, 1486, or 1083, the ISVD has at least 75% of the efficacy, preferably at least 80% or 90% of the efficacy, more preferably at least (essentially) the same efficacy T-cell activation compared to one of the VHHs with SEQ ID NOs: 793, 1505, 1092, 1085, 1078, 1486, or 1083, wherein the efficacy is monitored, e.g., via IFN-gamma production in an autologous Tetanus Toxoid recall assay (TT assay), e.g. the TT assay as described herein.

In some embodiments, the immunoglobulin single variable domains of the present technology specifically bind to human PD-1 with a dissociation constant (K_{D}) of 5×10⁻⁹ to 10⁻¹² moles/litre or less, and preferably 10⁻¹⁰ to 10⁻¹² moles/litre or less and more preferably 1×10⁻¹⁰ to 1×10⁻¹¹ moles/litre, such as 4.0×10⁻¹⁰ moles/litre, as determined by Surface Plasmon Resonance, e.g., measured at 25° C.

In some embodiments, the immunoglobulin single variable domains of the present technology specifically bind to human PD-1 with a kₒₙ-rate of between 10⁴ M⁻¹s⁻¹ to about 10⁷ M⁻¹s⁻¹, preferably between 10⁵ M⁻¹s⁻¹ and 10⁷ M⁻¹s⁻¹, more preferably between 10⁵ M⁻¹s⁻¹ and 10⁶ M⁻¹s⁻¹, such as between 10⁵ M⁻¹s⁻¹ and 5×10⁶ M⁻¹s⁻¹, such as 3.7×10⁵ M⁻¹s⁻¹, as determined by Surface Plasmon Resonance, e.g., measured at 25° C.

In some embodiments, the immunoglobulin single variable domains of the present technology specifically bind to human PD-1 with a k_{off} rate between 10⁻³ s⁻¹ (t1/2=0.69 s) and 10⁻⁷ s⁻¹ (providing a near irreversible complex with a t1/2 of multiple days), preferably between 10⁻³ s⁻¹ and 5×10⁻⁶ s⁻¹, more preferably between 5×10⁻⁴ s⁻¹ and 5×10⁻⁶ s⁻¹, such as between 1×10⁻⁴ s⁻¹ and 5×10⁻⁵ s⁻¹, such as 1.5×10⁻⁴ s⁻¹, as determined by Surface Plasmon Resonance, e.g., measured at 25° C.

In some embodiments, the immunoglobulin single variable domains of the present technology specifically bind to cyno PD-1 with a dissociation constant (K_{D}) of 5×10⁻⁹ to 10⁻¹² moles/litre or less, 10⁻⁹ to 10⁻¹² moles/litre or less, and preferably 10⁻¹⁰ to 10⁻¹² moles/litre or less and more preferably 1×10⁻¹⁰ to 5×10⁻¹⁰ moles/litre, such as 2.6×10⁻¹⁰ moles/litre, as determined by Surface Plasmon Resonance, e.g., measured at 25° C.

In some embodiments, the immunoglobulin single variable domains of the present technology specifically bind to cyno PD-1 with a kₒₙ-rate of between 10⁴ M⁻¹s⁻¹ to about 10⁷ M⁻¹s⁻¹, preferably between 10⁴ M⁻¹s⁻¹ and 10⁶ M⁻¹s⁻¹, more preferably between 10⁴ M⁻¹s⁻¹ and 5×10⁵ M⁻¹s⁻¹, such as between 10⁴ M⁻¹s⁻¹ and 10⁵ M⁻¹s⁻¹, such as 1.1×10⁴ M⁻¹s⁻¹, as determined by Surface Plasmon Resonance, e.g., measured at 25° C.

In some embodiments, the immunoglobulin single variable domains of the present technology specifically bind to cyno PD-1 with a k_{off} rate between 10⁻³ s⁻¹ (t1/2=0.69 s) and 10⁻⁸ s⁻¹ (providing a near irreversible complex with a t1/2 of multiple days), preferably between 10⁻³ s⁻¹ and 5×10⁻⁷ s⁻¹, more preferably between 10⁻³ s⁻¹ and 10⁻⁶ s⁻¹, such as between 1×10⁻³ s⁻¹ and 5×10⁻⁶ s⁻¹, such as 8.2×10⁻⁴ s⁻¹, as determined by Surface Plasmon Resonance, e.g., measured at 25° C.

In some embodiments, the immunoglobulin single variable domains of the present technology specifically bind to human PD-1 and cyno PD-1 with less than 10-fold difference in affinity (K_{D}).

In some embodiments, the immunoglobulin single variable domains of the present technology specifically bind to human and cyno PD-1 and do not bind to other members of the CD28 receptor family.

In some embodiments, the immunoglobulin single variable domains of the present technology specifically bind to human and cyno PD-1 and do not bind to CD28, CTLA-4, ICOS, and BTLA.

In the present technology, the binding of the ISVDs and polypeptides comprising the ISVD to PD-1 can also be measured in assays in which PD-1 is exposed on a cell surface (such as e.g. CHO Flp-In human PD-1 cells, CHO Flp-In cyno PD-1 cells). The Invitrogen™ Flp-In™ System allows integration and expression of the gene of interest (e.g. encoding human, cyno or mouse PD-1) in mammalian cells (e.g. CHO cells) at a specific genomic location. The Flp-In System involves introduction of a Flp Recombination Target (FRT) site into the genome of the mammalian cell line of choice. An expression vector containing the gene of interest is then integrated into the genome via Flp recombinasemediated DNA recombination at the FRT site (O'Gorman et al., 1991, Recombinase-Mediated Gene Activation and Site-Specific Integration in Mammalian Cells, Science 251, 1351-1355).

A preferred assay for measuring binding of the ISVD (or polypeptide comprising the ISVD) to PD-1 exposed on a cell surface is a FACS assay, such as e.g. the FACS assay as described in the examples, wherein binding of the ISVDs or polypeptides to PD-1 expressed on CHO is determined.

In some embodiments, the immunoglobulin single variable domains of the present technology specifically bind PD-1 and block the interaction of PD-1 with PD-L1. In some embodiments, the immunoglobulin single variable domains of the present technology specifically bind hPD-1 and block the interaction of hPD-1 with hPD-L1. In some embodiments, the immunoglobulin single variable domains of the present technology specifically bind cyno PD-1 and block the interaction of cyno PD-1 with cyno PD-L1.

In some embodiments, the immunoglobulin single variable domains of the present technology show a highly potency and/or efficacy (as a single variable domain) for binding PD-1 in competition with PD-L1. In some embodiments, the immunoglobulin single variable domains of the present technology can functionally block PD-1. More specifically, the ISVD can block the interaction between PD-1 and PD-L1. In some embodiments, the immunoglobulin single variable domains of the present technology antagonize an activity of PD-1.

In some embodiments, the immunoglobulin single variable domains of the present technology specifically bind to human PD-1 and block the interaction of human PD-1 with human PD-L1. For example, in FACS competition assay on CHO cells expressing human PD-1, with hPD-L1 as competitor, the immunoglobulin single variable domains of the present technology may have IC50 values of 5×10⁻⁸ M or lower, more preferably of 10⁻⁸ M or lower, or even of 10⁻⁹ M or lower. For example, in such

FACS competition assay, the immunoglobulin single variable domains of the present technology may have IC50 values between 10⁻¹¹ M and 10⁻⁸ M, such as between 10⁻¹⁰ M and 10⁻⁹ M, between 10⁻¹⁰ M and 10⁻⁸ M or between 10⁻⁹ M and 10⁻⁸ M, such as 6.07×10⁻⁹ M.

In some embodiments, the immunoglobulin single variable domains of the present technology specifically bind to cyno PD-1 and block the interaction of cyno PD-1 with cyno PD-L1 or human PD L1. For example, in a FACS competition assay on CHO cells expressing cyno PD-1, with human PD-L1 as competitor, the immunoglobulin single variable domains of the present technology may have IC50 values of 10⁻⁷ M or lower, more preferably of 5×10⁻⁸ M or lower, or even of 10⁻⁸ M or lower. For example, in such FACS competition assay, the immunoglobulin single variable domains of the present technology may have IC50 values between 10⁻¹¹ M and 10⁻⁷ M, such as between 10⁻¹⁰ M and 10⁻⁷ M, between 10⁻¹⁰ M and 10⁻⁸ M or between 10⁻⁸ M and 10⁻⁷ M, such as 1.19×10⁻⁸ M.

In some embodiments, the immunoglobulin single variable domains of the present technology block the binding of human PD-1 to human PD-L1 by at least 50%, such as at least 60%, 70%, 80%, 90%, 95% or even more, as determined by ligand competition, AlphaScreen, or competitive binding assays (such as competition ELISA or competition FACS).

In some embodiments, the immunoglobulin single variable domains of the present technology block the binding of cyno PD-1 to cyno PD-L1 by at least 50%, such as at least 60%, 70%, 80%, 90%, 95% or even more, as determined by ligand competition, AlphaScreen, or competitive binding assays (such as competition ELISA or competition FACS).

In some embodiments, the immunoglobulin single variable domains of the present technology show a highly potency for T cell activation as a single variable domain. T cell activation refers to a process in which mature T cells can express antigen-specific T cell receptors on their surface to recognize their cognate antigens and respond by entering the cell cycle, secreting cytokines or lytic enzymes, and initiating the cell-based functions of the immune system.

T-cell activation as used herein refers to one or more cellular response(s) of a T cell, e.g., a cytotoxic T cell, such as selected from: proliferation, differentiation, cytokine secretion, cytotoxic effector molecule release, cytotoxic activity, expression of activation markers, and redirected target cell lysis. The polypeptides of the invention are capable of inducing one or more of such responses. Suitable assays to measure T cell activation are known in the art, for instance as described in WO 99/54440 or by Schlereth et al. 2005 (Cancer Immunol. Immunother. 20:1-12), or as exemplified in the examples or below. For example, T cell activation can be measured by assessing (1) proliferation, (2) upregulation of activation markers (e.g., IL2RA/CD25) and/or (3) production of effector cytokines (IFNgamma, TNF-α).

In some embodiments, T cell activation by the ISVDs and polypeptides comprising the same can be monitored by upregulation of CD69, CD25 and various cell adhesion molecules, de novo expression and/or release of cytokines (e.g., IFN-γ, TNF-α, IL-6, IL-2, IL-4 and IL-10), upregulation of granzyme and perforin expression, and/or cell proliferation, membrane blebbing, activation of procaspases 3 and/or 7, fragmentation of nuclear DNA and/or cleavage of caspase substrate poly (ADPribose) polymerase.

In some embodiments, T cell activation is monitored by measurement of IFN-gamma release.

In some embodiments, T cell activation is measured by determining the expression of a number of molecules, namely CD25, CD69, CD71, and HLA-DR, also termed “activation antigens”, by activated proliferating lymphocytes on their surface.′ Such activation antigens are easily evidenced by immunofluorescence assay and flow cytometric analysis using monoclonal antibodies (mAbs) as specific reagents. By two- and three colour immunofluorescent staining it is also possible to identify, within bulk lymphocyte cultures, the T-cell subpopulations involved in the activation process.

In some embodiments, T cell activation can be measured by DNA synthesis assays. DNA synthesis assays use radioactively labelled 3H-thymine to detect DNA within cells. Newly proliferated cells incorporate the label, providing extremely sensitive and accurate detection of cell lines (see e.g., Madhavan 2007, J. Stem Cells Regen. Med. 3:12).

In some embodiments, T cell activation can be measured in a tetanus Toxoid recall assay (TT assay). The assay makes use of Lymphocytes that have previously been exposed to an antigen and that will show an enhanced response upon subsequent re-exposure. Primary lymphocytes from individuals vaccinated against tetanus are used to initiate an in vitro recall antigenic response. Stimulation of such lymphocytes with tetanus toxoid provides a useful assay method to assess the effects of candidate therapeutic molecules, such as checkpoint inhibitors, and as a model for analogous responses to tumor antigens. Lymphocytes are antigenically stimulated following culture with loaded antigen presenting cells (APCs). Antigen stimulated lymphocytes are cultured in the presence of checkpoint inhibitors to modulate activation response. Endpoints that are assessed in this assay include lymphocyte proliferation, activation markers, and cytokine production (Ticha et al. 2021, A cell-based in vitro assay for testing of immunological integrity of Tetanus toxoid vaccine antigen, Npj Vaccines 6:1-11). In some embodiments, IFN-gamma production is measured following activation of the T lymphocytes.

In some embodiments, the immunoglobulin single variable domains of the present technology have a potency (EC50 values) in a TT assay (e.g. as described in the Examples), monitored via IFN-gamma production, of 10⁷ M or lower, more preferably of 5×10⁻⁸ M or lower, or even of 10⁻⁸ M or lower. For example, in such TT assay, the immunoglobulin single variable domains of the present technology may have a potency (EC50 values) between 10⁻¹¹ M and 10⁻⁷ M, such as between 10⁻¹⁰ M and 10⁷ M, between 10⁻¹⁰ M and 10⁻⁸ M or between 10⁻⁸ M and 10⁻⁷ M, such as 4.32×10⁻⁸ M, 3.24×10⁻⁸ M or 7.1×10⁻³ M.

In some embodiments, a polypeptide comprising at least two immunoglobulin single variable domains of the present technology has a potency (EC50 values) in a TT assay (e.g. as described in the Examples), monitored via IFN-gamma production, of 10⁻⁸ M or lower, more preferably of 5×10⁻⁹ M or lower, or even of 10⁻⁹ M or lower. For example, in such TT assay, the immunoglobulin single variable domains of the present technology may have a potency (EC50 values) between 10⁻¹¹ M and 10⁻⁸ M, such as between 10⁻¹⁰ M and 10⁻⁸ M, between 10⁻¹⁰ M and 10⁻⁹ M or between 10⁻⁹ M and 10⁻⁸ M, such as 8.5×10⁻⁹ M, or 1.7×10⁻⁹ M.

In some embodiments, the immunoglobulin single variable domains of the present technology have an efficacy in a TT assay (e.g., as described in the Examples), monitored via IFN-gamma production, of at least 50%, such as at least 60%, 70%, 80%, 90%, 95%.

The T Cell Activation Bioassay (NFAT) is a bioluminescent cell-based assay that consists of a genetically engineered Jurkat T cell line that expresses a luciferase reporter driven by an NFAT-response element (NFAT-RE). When the TCR/CD3 Effector Cells (NFAT) are engaged, the TCR transduces intracellular signals resulting in NFAT-RE-mediated luminescence. The bioluminescent signal is detected and quantified using Bio-Glo™ Luciferase Assay System and a standard luminometer, such as the GloMax® Discover System.

In some embodiments, the immunoglobulin single variable domains of the present technology have a potency (IC50 values) in a NFAT assay (e.g., as described in the Examples) of 10⁻⁷ M or lower, of 10⁻⁸ M or lower, more preferably of 5×10⁻⁹ M or lower, or even of 10⁻⁹ M or lower, or even 5×10⁻¹⁰ or lower.

For example, in such NFAT assay, the immunoglobulin single variable domains of the present technology may have a potency (IC50 values) between 10⁻¹⁰ M and 10⁻⁷ M, such as between 10⁻¹⁰ M and 10⁻⁸ M or between 10⁻⁹ M and 10⁻⁸ M, such as 5.82×10⁻⁹ M or 1.5×10⁻⁹ M.

In some embodiments, the immunoglobulin single variable domains of the present technology block the binding of human PD-L1 to human PD-1 by at least 50%, such as at least 60%, 70%, 80%, 90%, 95% or even more, as determined in a NFAT assay.

In some embodiments, the immunoglobulin single variable domains of the present technology block the binding of human or cyno PD-L1 to cyno PD-1 by at least 50%, such as at least 60%, 70%, 80%, 90%, 95% or even more, as determined in a NFAT assay.

In another embodiment, when the ISVD specifically binding to human PD-1 has a sequence identity of more than 90%, such as more than 95% or more than 99%, with one of the VHHs with SEQ ID NOS: 793, 1505, 1092, 1085, 1078, 1486, or 1083, the ISVD blocks the interaction of hPD-1 with (h) PD-L1 with (essentially) the same % inhibition or more compared to one of the VHHs with SEQ ID NOs: 793, 1505, 1092, 1085, 1078, 1486, or 1083, wherein the % inhibition is determined in a NFAT assay.

### 5.2.3 Embodiment 3, Family A0294037F03

There is provided an immunoglobulin single variable domain (ISVD) specifically binding to human PD1 protein, that essentially consists of 4 framework regions (FR1 to FR4, respectively) and 3 complementarity determining regions (CDR1 to CDR3, respectively), wherein the ISVD binds to an (discontinuous) epitope on human PD-1 protein, wherein at least one amino acid residue located in at least one of the CDRs is capable of interacting with at least one amino acid residue in at least one, preferably in each, of the following stretches (of the ectodomain) of human PD-1 protein, when numbered in accordance with SEQ ID NO: 4703: W32, Y68-M70, K78, and I126-R139, preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, the at least one amino acid residue in at least one, preferably in each stretch (of the ectodomain) of human PD-1 protein is selected from:
For stretch Y68-M70: Y68, and M70;
For stretch I126-R139: I126, L128, P130, A132, Q133, I134, K135, E136, and R139, when numbered in accordance with SEQ ID NO: 4703.

In some embodiments, at least one amino acid residue located in at least one of the CDRs is capable of interacting with all of following amino acid residues in stretch Y68-M70 (of the ectodomain) of human PD-1 protein, when numbered in accordance with SEQ ID NO: 4703: Y68, and M70.

In some embodiments, at least one amino acid residue located in at least one of the CDRs is capable of interacting with at least two, at least three, at least four, at least five, at least six, at least seven, at least eight, or all of following amino acid residues in stretch I126-R139 (of the ectodomain) of human PD-1 protein when numbered in accordance with SEQ ID NO: 4703: I126, L128, P130, A132, Q133, I134, K135, E136, and R139, and optionally K131.

In some embodiments, the at least one amino acid residue in each stretch (of the ectodomain) of human PD-1 protein is K131, when numbered in accordance with SEQ ID NO: 4703.

In some embodiments, at least one amino acid residue located in at least one of the CDRs is capable of interacting with at least two, at least three, at least four, at least five, at least six, at least seven, at least eight, or all of following amino acid residues in stretch I126-R139 (of the ectodomain) of human PD-1 protein when numbered in accordance with SEQ ID NO: 4703: I126, L128, P130, K131, A132, Q133, I134, K135, E136, and R139.

In some embodiments, at least one amino acid residue located in at least one of the CDRs is capable of forming hydrogen bonds with at least one, preferably all, of the following amino acid residues of human PD-1 protein, when numbered in accordance with SEQ ID NO: 4703: A132, and Q133, preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, at least one amino acid residue located in at least one of the CDRs is capable of forming hydrogen bonds with at least one, preferably all, of the following amino acid residues of human PD-1 protein, when numbered in accordance with SEQ ID NO: 4703: M70, K78, K131, A132, Q133, I134, K135, and E136, and optionally K131, preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, the at least one amino acid residue located in at least one of the CDRs is capable of interacting with an (discontinuous) epitope on human PD-1 comprising at least one amino acid residue in the following stretch (of the ectodomain) of human PD-1 protein: Y68-M70, when numbered in accordance with SEQ ID NO: 4703.

In some embodiments, the at least one amino acid residue in said stretch (of the ectodomain) of human PD-1 protein is selected from: Y68, and M70, when numbered in accordance with SEQ ID NO: 4703.

In some embodiments, the at least one amino acid residue located in at least one of the CDRs is capable of interacting with an (discontinuous) epitope on human PD-1 comprising at least one amino acid residue in the following stretch (of the ectodomain) of human PD-1 protein I126-R139, when numbered in accordance with SEQ ID NO: 4703.

In some embodiments, at least one amino acid residue in said stretch (of the ectodomain) of human PD-1 protein is selected from: I126, L128, P130, A132, Q133, I134, K135, E136, R139 and optionally K131, when numbered in accordance with SEQ ID NO: 4703.

In some embodiments, at least one amino acid residue located in at least one of the CDRs is capable of interacting with at least one, at least two, at least three, at least four, at least five, at least six, at least seven, at least ten, at least fifteen, preferably all, of the following amino acid residues of human PD-1 protein, when numbered in accordance with SEQ ID NO: 4703: W32, Y68, M70, I126, L128, P130, A132, Q133, I134, K135, E136, R139, and E136 and optionally K131, preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, the at least one amino acid residue located in at least one of the CDRs capable of interacting with at least one amino acid residue of human PD-1 protein is located in CDR1, CDR2 and/or CDR3.

In some embodiments:
the amino acid residue at position 100 (Kabat numbering) is capable of interacting, preferably by forming a hydrogen bond, with Q133 in PD-1;
the amino acid residue at position 100b (Kabat numbering) is capable of interacting, preferably by forming a hydrogen bond, with A132 in PD-1;
and optionally wherein:
the amino acid residue at position 99 (Kabat numbering) is capable of interacting, preferably by forming a hydrogen bond, with K135 in PD-1;
the amino acid residue at position 54 (Kabat numbering) is capable of interacting, preferably by forming a hydrogen bond, with M70 in PD-1;
the amino acid residue at position 32 (Kabat numbering) is capable of interacting, preferably by forming a hydrogen bond, with I134 in PD-1;
the amino acid residue at position 54 (Kabat numbering) is capable of interacting, preferably by forming a hydrogen bond, with E136 in PD-1;
the amino acid residue at position 52 (Kabat numbering) is capable of interacting, preferably by forming a hydrogen bond, with E136 in PD-1;
the amino acid residue at position 57 (Kabat numbering) is capable of interacting, preferably by forming a hydrogen bond, with K78 in PD-1; and/or the amino acid residue at position 54 (Kabat numbering) is capable of interacting, preferably by forming a hydrogen bond, with K131 in PD-1;
preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, two amino acids are considered to interact when the distance between them is 4.0 Å or less.

In some embodiments, the interacting amino acid residue(s) in the ISVD and in human PD-1 protein are identified by X-ray crystallography by:
Mixing human PD-1 protein, and the ISVD in PBS1X buffer;
Concentrating the protein complex to 12 mg/ml;
Crystallizing by hanging drop vapor diffusion using 1 M Na Acetate, 1M (NH₄)₂HPO₄, pH 4.5 at 19° C.;
Collecting X-ray diffraction data;
Processing the collected data,
preferably as described in Example 13.

In some embodiments, the (discontinuous) epitope comprises or consists of the following amino acid residues of human PD-1 protein, when numbered in accordance with SEQ ID NO: 4703: W32, Y68, M70, I126, L128, P130, K131, A132, Q133, I134, K135, E136, R139.

In some embodiments, the ISVD cross-blocks the binding to human PD-1 protein of a polypeptide having an amino acid sequence selected from SEQ ID NOs: 2857-3662, preferably the ISVD cross-blocks the binding to human PD-1 protein of a polypeptide having an amino acid sequence selected from SEQ ID NO: 2859, or the binding of the ISVD to human PD-1 protein is cross-blocked by a polypeptide having an amino acid sequence selected from SEQ ID NOs: 2857-3662, preferably SEQ ID NO: 2859.

There is provided an immunoglobulin single variable domain (ISVD) specifically binding human PD-1 protein, that essentially consists of 4 framework regions (FR1 to FR4, respectively) and 3 complementarity determining regions (CDR1 to CDR3, respectively), wherein the ISVD comprises an amino acid residue capable of forming hydrogen bonds at at least one, preferably at all, of the following positions: 100, 100b (Kabat numbering) and optionally further at position 32, 52, 54, 57, 99 (Kabat numbering), preferably wherein the ISVD comprises at least one, preferably all of the following amino acid residues at the following positions: S100, Y100b (Kabat numbering) and optionally N32, S52, N54, T57, S99 (Kabat numbering).

In some embodiments, the ISVD comprises an amino acid residue capable of forming hydrogen bonds at position 45 (Kabat numbering, preferably wherein the ISVD comprises R45 (Kabat numbering).

In some embodiments, the ISVD comprises at least one, preferably all of the following amino acid residues at the following positions (Kabat numbering): N32, and A33, preferably at least N32.

In some embodiments, the ISVD comprises at least one, preferably all of the following amino acid residues at the following positions (Kabat numbering): L50, S52, N54, 156, T57, and Y58, preferably at least S52, N54, and T57.

In some embodiments, the ISVD comprises at least one, preferably all of the following amino acid residues at the following positions (Kabat numbering): E95, Y97, S99, S100, W100a, Y100b, preferably at least S99, S100, and Y100b.

In some embodimentss:
the CDR1 (AbM definition) consists of the following amino acid sequence X₁X₂X₃X₄X₅X₆NAMA (SEQ ID NO: 4699); wherein
   X₁ is selected from any amino acid, preferably X₁ is selected from G, T, D, F, S, R, E, H, I, A, V, or K;
   X₂ is selected from any amino acid, preferably X₂ is selected from R, M, D, H, Y, F, V, L, Q, K, P, A, I, or G;
   X₃ is selected from any amino acid, preferably X₃ is selected from M, F, R, S, I, D, V, K, T, Y, A, E, or G;
   X₄ is selected from any amino acid, preferably X₄ is selected from H, R, S, E, P, G, Q, K, A, D, T, V, I, F, Y, or L;
   X₅ is selected from any amino acid, preferably X₅ is selected from S, T, K, G, I, Y, H, F, D, E, V, P, and L;
   X₆ is selected from any amino acid, preferably X₆ is selected from I, V, S, K, E, D, Q, R, P, G, T, L, or F; and/or
the CDR2 (AbM definition) consists of the following amino acid sequence LX₁SX₂X₃NX₄ITY (SEQ ID NO: 4700); wherein:
   X₁ is selected from any amino acid, preferably X₁ is selected from I, G, Y, A, Q, H, T, L, S, D, R, K, or P;
   X₂ is selected from any amino acid, preferably X₂ is selected W, L, K, S, Q, V, D, L, H, T, E, I, A, R, P, Y, G, or F;
   X₃ is selected from any amino acid, preferably X₃ is selected from G, L, T, Y, R, V, P, Q, F, A, and S;
   X₄ is selected from any amino acid, preferably X₄ is selected from G, Q, F, S, I, H, R, E, V, P, L, D, T, or A; and/or
the CDR3 (AbM definition) consists of the following amino acid sequence EX₁Y X₂SSWYX₃X₄ (SEQ ID NO: 4701); wherein:
   X₁ is selected from any amino acid, preferably X₁ is selected from S, F, A, Y, E, I, D, K, G, R,P, Q, T, H, or L;
   X₂ is selected from any amino acid, preferably X₂ is selected G, L, I, H, S, V, R, P, E, A, K, Y, T, F, A, D, H, or R;
   X₃ is selected from any amino acid, preferably X₃ is selected from D, N, A, S, L, K, T, F, I, G, Y, R, V, E, or H, or is absent;
   X₄ is selected from any amino acid, preferably X₄ is selected from S, D, V, H, A, L, E, T, Y, K, F, I, Q, R, G, or P, or is absent.

In some embodiments, at least one, preferably all of the following amino acid residues of the ISVD at the following positions: S99, S100, Y100b (Kabat numbering), optionally N32, S52, N54, T57 (Kabat numbering), and further optionally R45, are capable of forming hydrogen bonds with at least one of the amino acid residues of human PD-1 protein, preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, at least one, preferably all, of the following amino acid residues of the ISVD at the following positions: N32, S52, N54, T57, S99, S100, and Y100b, (Kabat numbering) is capable of interacting with at least one, preferably all of the following amino acid residues of human PD-1 protein, when numbered in accordance with SEQ ID NO: 4703: M70, K78, A132, Q133, I134, K135, and E136, preferably wherein:
S99 in the ISVD (Kabat numbering) interacts, preferably by forming a hydrogen bond, with K135 in PD-1;
S100 in the ISVD (Kabat numbering) interacts, preferably by forming a hydrogen bond, with Q133 in PD-1;
Y100b in the ISVD (Kabat numbering) interacts, preferably by forming a hydrogen bond, with A132 in PD-1;
N54 in the ISVD (Kabat numbering) interacts, preferably by forming a hydrogen bond, with M70 in PD-1;
N32 in the ISVD (Kabat numbering) interacts, preferably by forming a hydrogen bond, with I134 in PD-1;
N54 in the ISVD (Kabat numbering) interacts, preferably by forming a hydrogen bond, with E136 in PD-1; and/or
S52 in the ISVD (Kabat numbering) interacts, preferably by forming a hydrogen bond, with E136 in PD-1; and/or
T57 in the ISVD (Kabat numbering) interacts, preferably by forming a hydrogen bond, with K78 in PD-1;
preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, R45 (Kabat numbering) is capable of interacting with K131 of human PD-1 protein, when numbered in accordance with SEQ ID NO: 4703, preferably R45 in the ISVD (Kabat numbering) interacts, preferably by forming a hydrogen bond, with K131 in PD-1; preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, the ISVD comprises at least one, preferably all of the following amino acid residues at the following positions (Kabat numbering): S100, and Y100b.

In some embodiments, the ISVD comprises at least one, at least two, at least three, at least four, at least five, at least six, at least seven, preferably all of the following amino acid residues at the following positions (Kabat numbering): N32, S52, N54, T57, S99, S100, and Y100b, and optionally R45.

In some embodiments, the ISVD comprises at least one, at least two, at least three, at least four, at least five, at least six, at least seven, at least eight, at least nine, at least ten, at least eleven, at least twelve, at least thirteen, preferably all of the following amino acid residues at the following positions (Kabat numbering): N32, A33, L50, S52, N54, 156, T57, Y58, E95, Y97, S99, S100, W100a, and Y100b.

In some embodiments, the ISVD comprises at least one, at least two, at least three, at least four, at least five, at least six, at least seven, at least eight, at least nine, at least ten, at least eleven, at least twelve, at least thirteen, at least fourteen, at least fifteen, at least sixteen preferably all of the following amino acid residues at the following positions (Kabat numbering): N32, A33, F37, R45, F47, L50, S52, N54, 156, T57, Y58, E95, Y97, S99, S100, W100a, Y100b.

In some embodiments, S100, and Y100b (Kabat numbering) of the ISVD are capable of interacting with at least one of the following amino acids of human PD-1 protein, when numbered in accordance with SEQ ID NO: 4703: A132, and Q133, preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, N32, S52, N54, T57, S99, S100, and Y100b (Kabat numbering) of the ISVD are capable of interacting with at least one of the following amino acids of human PD-1 protein, when numbered in accordance with SEQ ID NO: 4703: W32, Y68, M70, K78, I126, L128, P130, A132, Q133, I134, K135, E136, and R139, preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, N32, R45, S52, N54, T57, S99, S100, and Y100b (Kabat numbering) of the ISVD are capable of interacting with at least one of the following amino acids of human PD-1 protein, when numbered in accordance with SEQ ID NO: 4703: W32, Y68, M70, K78, I126, L128, P130, K131, A132, Q133, I134, K135, E136, R139, and E136, preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, N32, A33, F37, R45, F47, L50, S52, N54, 156, T57, Y58, E95, Y97, S99, S100, W100a, Y100b (Kabat numbering) of the ISVD are capable of interacting with the following amino acids of human PD-1 protein, when numbered in accordance with SEQ ID NO: 4703: W32, Y68, M70, K78, 1126, L128, P130, K131, A132, Q133, I134, K135, E136, and R139, preferably when the ISVD and human PD-1 protein are mixed in PBS1X buffer and crystallized by hanging drop vapor diffusion using 1 M Na Acetate, 1M (NH₄)₂HPO₄, pH 4.5 at 19° C.

In some embodiments, two amino acids are considered to interact when the distance between them is 4.0 Å or less.

In some embodiments, the interacting amino acid residue(s) in the ISVD and in human PD-1 protein are identified by X-Ray crystallography by:
Mixing human PD-1 protein, and the ISVD in PBS1X buffer;
Concentrating the protein complex to 8 mg/ml;
Crystallizing by hanging drop vapor diffusion using 1 M Na Acetate, 1M (NH₄)₂HPO₄, pH 4.5 at 19° C.;
Collecting X-ray diffraction data;
Processing the collected data,
preferably as described in Example 13.

There is provided an immunoglobulin single variable domain (ISVD) specifically binding to human PD1 protein, that essentially consists of 4 framework regions (FR1 to FR4, respectively) and 3 complementarity determining regions (CDR1 to CDR3, respectively), wherein the ISVD binds to an (discontinuous) epitope on human PD-1, wherein the ISVD cross-blocks the binding to human PD-1 protein of a polypeptide with the amino acid sequence of SEQ ID Nos: 2857-3662, preferably SEQ ID NO: 2859, or wherein the ISVD is cross-blocked from binding to human PD-1 protein by a polypeptide with the amino acid sequence of SEQ ID Nos: 2857-3662, preferably SEQ ID NO: 2859.

In some embodiments, the ISVD competes with a polypeptide with the amino acid sequence of SEQ ID NO: 2857-3662, preferably SEQ ID NO: 2859, for binding human PD-1 protein, as measured in a competition assay, such as a FACS competition assay.

Specific examples of ISVDs specifically binding to PD-1 are ISVDs that comprises 4 framework regions (FR1 to FR4, respectively) and three complementarity determining regions (CDR1 to CDR3, respectively), wherein:
CDR1 (AbM numbering) consists of an amino acid sequence selected from:
   a) the amino acid sequence of X₁X₂X₃X₄X₅X₆X₇X₈MX₉ (SEQ ID NO: 4608), wherein X₁ is selected from G, D, F, S, R, E, H, I, A, V, or K, X₂ is selected from R, M, D, H, Y, F, V, L, Q, K, P, A, I, or G, wherein X₃ is selected from M, F, R, S, I, D, V, K, T, Y, A, E, or G, X₄ is selected from H, R, S, E, P, G, Q, K, A, D, T, V, I, F, Y, or L, X₅ is selected from S, T, K, G, I, Y, H, F, D, E, V, P, and L, X₆ is selected from I, V, S, K, E, D, Q, R, P, G, T, L, or F, X₇ is selected from N, G, V, E, L, I, Y, Q, T, F, P, or H, X₈ is selected from A, D, E, E, I, H, S, V, L, K, G, T, or Y, and X₉ is selected from A, H, F, K, L, D, P, T, S, V, G, Y, R, or Q;
   b) amino acid sequences that have at least 80% amino acid identity with the amino acid sequence of X₁X₂X₃X₄X₅X₆X₇X: MX₉ (SEQ ID NO: 4608), wherein X₁ is selected from G, D, F, S, R, E, H, I, A, V, or K, X₂ is selected from R, M, D, H, Y, F, V, L, Q, K, P, A, I, or G, wherein X₃ is selected from M, F, R, S, I, D, V, K, T, Y, A, E, or G, X₄ is selected from H, R, S, E, P, G, Q, K, A, D, T, V, I, F, Y, or L, X₅ is selected from S, T, K, G, I, Y, H, F, D, E, V, P, and L, X₆ is selected from I, V, S, K, E, D, Q, R, P, G, T, L, or F, X₇ is selected from N, G, V, E, L, I, Y, Q, T, F, P, or H, X₈ is selected from A, D, E, E, I, H, S, V, L, K, G, T, or Y, and X₉ is selected from A, H, F, K, L, D, P, T, S, V, G, Y, R, or Q; and
   c) amino acid sequences that have 3, 2, or 1 amino acid difference with the amino acid sequences of X₁X₂X₃X₄X₅X₆X₇X₈MX₉ (SEQ ID NO: 4608), wherein X₁ is selected from G, D, F, S, R, E, H, I, A, V, or K, X₂ is selected from R, M, D, H, Y, F, V, L, Q, K, P, A, I, or G, wherein X₃ is selected from M, F, R, S, I, D, V, K, T, Y, A, E, or G, X₄ is selected from H, R, S, E, P, G, Q, K, A, D, T, V, I, F, Y, or L, X₅ is selected from S, T, K, G, I, Y, H, F, D, E, V, P, and L, X₆ is selected from I, V, S, K, E, D, Q, R, P, G, T, L, or F, X₇ is selected from N, G, V, E, L, I, Y, Q, T, F, P, or H, X₈ is selected from A, D, E, E, I, H, S, V, L, K, G, T, or Y, and X₉ is selected from A, H, F, K, L, D, P, T, S, V, G, Y, R, or Q;
      and
CDR2 (AbM numbering) consists of an amino acid sequence selected from:
   a) the amino acid sequence of X₁X₂X₃X₄X₅X₆X₇X₈X₉X₁₀ (SEQ ID NO: 4609), wherein X₁ is selected from L, V, I, T, F, E, Q, D, R, G, S, A, P, H, or K, X₂ is selected from I, G, Y, A, Q, H, T, L, S, D, R, K, or P, and X₃ is selected from S, G, L, V, K, F, I, T, E, A, D, H, P, Q, or R, X₄ is selected from W, L, K, S, Q, V, D, L, H, T, E, I, A, R, P, Y, G, or F, X₅ is selected from G, L, T, Y, R, V, P, Q, F, A, and S, X₆ is selected from N, D, H, T, A, F, E, S, V, Q, I, L, R, Y, G, X₇ is selected from G, Q, F, S, I, H, R, E, V, P, L, D, T, or A, X₈ is selected from I, Q, T, L, P, V, Y, H, D, R, E, A, G, F, S, or K, X₉ is selected from T, L, D, Q, I, S, G, F, E, A, Y, P, V, or K, and X₁₀ is selected from Y, P, R, T, G, A, H, S, Q, D, E, L, K, I, F, V, or H;
   b) amino acid sequences that have at least 80% amino acid identity with the amino acid sequence of X₁X₂X₃X₄X₅X₆X₇X₈X₉X₁₀ (SEQ ID NO: 4609), wherein X₁ is selected from L, V, I, T, F, E, Q, D, R, G, S, A, P, H, or K, X₂ is selected from I, G, Y, A, Q, H, T, L, S, D, R, K, or P, and X₃ is selected from S, G, L, V, K, F, I, T, E, A, D, H, P, Q, or R, X₄ is selected from W, L, K, S, Q, V, D, L, H, T, E, I, A, R, P, Y, G, or F, X₅ is selected from G, L, T, Y, R, V, P, Q, F, A, and S, X₆ is selected from N, D, H, T, A, F, E, S, V, Q, I, L, R, Y, G, X₇ is selected from G, Q, F, S, I, H, R, E, V, P, L, D, T, or A, X₈ is selected from I, Q, T, L, P, V, Y, H, D, R, E, A, G, F, S, or K, X₉ is selected from T, L, D, Q, I, S, G, F, E, A, Y, P, V, or K, and X₁₀ is selected from Y, P, R, T, G, A, H, S, Q, D, E, L, K, I, F, V, or H;
   c) amino acid sequences that have 3, 2, or 1 amino acid difference with the amino acid sequence of X₁X₂X₃X₄X₅X₆X₇X₈X₉X₁₀ (SEQ ID NO: 4609), wherein X₁ is selected from L, V, I, T, F, E, Q, D, R, G, S, A, P, H, or K, X₂ is selected from I, G, Y, A, Q, H, T, L, S, D, R, K, or P, and X₃ is selected from S, G, L, V, K, F, I, T, E, A, D, H, P, Q, or R, X₄ is selected from W, L, K, S, Q, V, D, L, H, T, E, I, A, R, P, Y, G, or F, X₅ is selected from G, L, T, Y, R, V, P, Q, F, A, and S, X₆ is selected from N, D, H, T, A, F, E, S, V, Q, I, L, R, Y, G, X₇ is selected from G, Q, F, S, I, H, R, E, V, P, L, D, T, or A, X₈ is selected from I, Q, T, L, P, V, Y, H, D, R, E, A, G, F, S, or K, X₉ is selected from T, L, D, Q, I, S, G, F, E, A, Y, P, V, or K, and X₁₀ is selected from Y, P, R, T, G, A, H, S, Q, D, E, L, K, I, F, V, or H;
      and
CDR3 (AbM numbering) consists of an amino acid sequence selected from:
   a) the amino acid sequence of X₁X₂X₃X₄X₅X₆X₇X₈X₉X₁₀ (SEQ ID NO: 4610), wherein X₁ is selected from E, P, D, A, K, I, Y, H, R, F, S, T, V, G, or Q, X₂ is selected S, F, A, Y, E, I, D, K, G, R,P, Q, T, H, or L, and X₃ is selected Y, D, F, E, R, K, I, T, A, Q, S, V, H, G, or P, X₄ is selected from G, L, I, H, S, V, R, P, E, A, K, Y, T, F, A, D, H, or R, X₅ is selected from S, D, K, Y, A, L, I, P, V, Q, E, R, T, G, or F, X₆ is selected from S, T, K, P,H, I, G, V, Q, R, D, L, or E, X₇ is selected from W, C, P, K, Y, V, F, A, I, T, L, H, G, Q, R, D, or E, X₈ is selected from Y, F, Q, P, K, T, R, D, I, E, L, H, V, A, G, or S, and X₉ is selected from D, N, A, S, L, K, T, F, I, G, Y, R, V, E, or H, or is absent, and X₁₀ is selected from S, D, V, H, A, L, E, T, Y, K, F, I, Q, R, G, or P, or is absent;
   b) amino acid sequences that have at least 80% amino acid identity with the amino acid sequence X₁X₂X₃X₄X₅X₆X₇X₈X₉X₁₀ (SEQ ID NO: 4610), wherein X₁ is selected from E, P, D, A, K, I, Y, H, R, F, S, T, V, G, or Q, X₂ is selected S, F, A, Y, E, I, D, K, G, R,P, Q, T, H, or L, and X₃ is selected Y, D, F, E, R, K, I, T, A, Q, S, V, H, G, or P, X₄ is selected from G, L, I, H, S, V, R, P, E, A, K, Y, T, F, A, D, H, or R, X₅ is selected from S, D, K, Y, A, L, I, P, V, Q, E, R, T, G, or F, X₆ is selected from S, T, K, P,H, I, G, V, Q, R, D, L, or E, X₇ is selected from W, C, P, K, Y, V, F, A, I, T, L, H, G, Q, R, D, or E, X₈ is selected from Y, F, Q, P, K, T, R, D, I, E, L, H, V, A, G, or S, and X₉ is selected from D, N, A, S, L, K, T, F, I, G, Y, R, V, E, or H, or is absent, and X₁₀ is selected from S, D, V, H, A, L, E, T, Y, K, F, I, Q, R, G, or P, or is absent;
   c) amino acid sequences that have 3, 2, or 1 amino acid difference with the amino acid sequence of X₁X₂X₃X₄X₅X₆X₇X₈X₉X₁₀ (SEQ ID NO: 4610), wherein X₁ is selected from E, P, D, A, K, I, Y, H, R, F, S, T, V, G, or Q, X₂ is selected S, F, A, Y, E, I, D, K, G, R,P, Q, T, H, or L, and X₃ is selected Y, D, F, E, R, K, I, T, A, Q, S, V, H, G, or P, X₄ is selected from G, L, I, H, S, V, R, P, E, A, K, Y, T, F, A, D, H, or R, X₅ is selected from S, D, K, Y, A, L, I, P, V, Q, E, R, T, G, or F, X₆ is selected from S, T, K, P,H, I, G, V, Q, R, D, L, or E, X₇ is selected from W, C, P, K, Y, V, F, A, I, T, L, H, G, Q, R, D, or E, X₈ is selected from Y, F, Q, P, K, T, R, D, I, E, L, H, V, A, G, or S, and X₉ is selected from D, N, A, S, L, K, T, F, I, G, Y, R, V, E, or H, or is absent, and X₁₀ is selected from S, D, V, H, A, L, E, T, Y, K, F, I, Q, R, G, or P, or is absent.

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NO: 4608, SEQ ID NO: 4609 or SEQ ID NO: 4610.

In one embodiment, the ISVD comprises CDRs (AbM numbering) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 2857-3662.

In one embodiment, the ISVD essentially consists of 4 framework regions (FR1 to FR4, respectively) and 3 complementarity determining regions (CDR1 to CDR3, respectively), in which:
CDR1 (AbM numbering) has an amino acid sequence selected from:
a) the amino acid sequence of GRXHSINAMA (SEQ ID NO: 4704), wherein X is selected from M, V and T;
b) amino acid sequences that have at least 80% amino acid identity with the amino acid sequence of GRXHSINAMA (SEQ ID NO: 4704), wherein X is selected from M, V and T;
c) amino acid sequences that have 3, 2, or 1 amino acid difference with the amino acid sequences of GRXHSINAMA (SEQ ID NO: 4704), wherein X is selected from M, V and T;
and
CDR2 (AbM numbering) has an amino acid sequence selected from:
   a) the amino acid sequence of LISWGXGITY (SEQ ID NO: 4705), wherein X is selected from N and F;
b) amino acid sequences that have at least 80% amino acid identity with the amino acid sequence of LISWGXGITY (SEQ ID NO: 4705), wherein X is selected from N and F;
c) amino acid sequences that have 3, 2, or 1 amino acid difference with the amino acid sequence of LISWGXGITY (SEQ ID NO: 4705), wherein X is selected from N and F;
and
CDR3 (AbM numbering) has an amino acid sequence selected from:
a) the amino acid sequence of ESYGXSWYDS (SEQ ID NO: 4706); wherein X is selected from S and A;
b) amino acid sequences that have at least 80% amino acid identity with the amino acid sequence of ESYGXSWYDS (SEQ ID NO: 4706); wherein X is selected from S and A;
c) amino acid sequences that have 3, 2, or 1 amino acid difference with the amino acid sequence of ESYGXSWYDS (SEQ ID NO: 4706); wherein X is selected from S and A.

In one embodiment, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 2498;
   b) amino acid sequences that have at least 80% amino acid identity with the amino acid sequence of SEQ ID NO: 2498; and
   c) amino acid sequences that have 3, 2, or 1 amino acid difference with the amino acid sequences of SEQ ID NO: 2498;
and
CDR2 (AbM definition) has an amino acid sequence selected from:
   a) the amino acid sequence of SEQ ID NO: 2451;
   b) amino acid sequences that have at least 80% amino acid identity with the amino acid sequence of SEQ ID NO: 2451;
   c) amino acid sequences that have 3, 2, or 1 amino acid difference with the amino acid sequence of SEQ ID NO: 2451;
and
CDR3 (AbM definition) has an amino acid sequence selected from:
   a) the amino acid sequence of SEQ ID NOs: 2804;
   b) amino acid sequences that have at least 80% amino acid identity with the amino acid sequence of SEQ ID NO: 2804;
   c) amino acid sequences that have 3, 2, or 1 amino acid difference with the amino acid sequence of SEQ ID NO: 2804.

In one embodiment, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 2552;
   b) amino acid sequences that have at least 80% amino acid identity with the amino acid sequence of SEQ ID NO: 2552; and
   c) amino acid sequences that have 3, 2, or 1 amino acid difference with the amino acid sequences of SEQ ID NO: 2552;
and
CDR2 (AbM definition) has an amino acid sequence selected from:
   a) the amino acid sequence of SEQ ID NO: 2652;
   b) amino acid sequences that have at least 80% amino acid identity with the amino acid sequence of SEQ ID NO: 2652;
   c) amino acid sequences that have 3, 2, or 1 amino acid difference with the amino acid sequence of SEQ ID NO: 2652;
and
CDR3 (AbM definition) has an amino acid sequence selected from:
   a) the amino acid sequence of SEQ ID NOs: 2741;
   b) amino acid sequences that have at least 80% amino acid identity with the amino acid sequence of SEQ ID NO: 2741;
   c) amino acid sequences that have 3, 2, or 1 amino acid difference with the amino acid sequence of SEQ ID NO: 2741.

In one embodiment, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 2551;
   b) amino acid sequences that have at least 80% amino acid identity with the amino acid sequence of SEQ ID NO: 2551; and
   c) amino acid sequences that have 3, 2, or 1 amino acid difference with the amino acid sequences of SEQ ID NO: 2551;
and
CDR2 (AbM definition) has an amino acid sequence selected from:
   a) the amino acid sequence of SEQ ID NO: 2652;
   b) amino acid sequences that have at least 80% amino acid identity with the amino acid sequence of SEQ ID NO: 2652;
   c) amino acid sequences that have 3, 2, or 1 amino acid difference with the amino acid sequence of SEQ ID NO: 2652;
and
CDR3 (AbM definition) has an amino acid sequence selected from:
   a) the amino acid sequence of SEQ ID NOs: 2741;
   b) amino acid sequences that have at least 80% amino acid identity with the amino acid sequence of SEQ ID NO: 2741;
   c) amino acid sequences that have 3, 2, or 1 amino acid difference with the amino acid sequence of SEQ ID NO: 2741.

In one embodiment, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 2552;
   b) amino acid sequences that have at least 80% amino acid identity with the amino acid sequence of SEQ ID NO: 2552; and
   c) amino acid sequences that have 3, 2, or 1 amino acid difference with the amino acid sequences of SEQ ID NO: 2552;
and
CDR2 (AbM definition) has an amino acid sequence selected from:
   a) the amino acid sequence of SEQ ID NO: 2652;
   b) amino acid sequences that have at least 80% amino acid identity with the amino acid sequence of SEQ ID NO: 2652;
   c) amino acid sequences that have 3, 2, or 1 amino acid difference with the amino acid sequence of SEQ ID NO: 2652;
and
CDR3 (AbM definition) has an amino acid sequence selected from:
   a) the amino acid sequence of SEQ ID NOs: 2741;
   b) amino acid sequences that have at least 80% amino acid identity with the amino acid sequence of SEQ ID NO: 2741;
   c) amino acid sequences that have 3, 2, or 1 amino acid difference with the amino acid sequence of SEQ ID NO: 2741.

In one embodiment, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of SEQ ID NO: 2551;
   b) amino acid sequences that have at least 80% amino acid identity with the amino acid sequence of SEQ ID NO: 2551; and
   c) amino acid sequences that have 3, 2, or 1 amino acid difference with the amino acid sequences of SEQ ID NO: 2551;
and
CDR2 (AbM definition) has an amino acid sequence selected from:
   a) the amino acid sequence of SEQ ID NO: 2652;
   b) amino acid sequences that have at least 80% amino acid identity with the amino acid sequence of SEQ ID NO: 2652;
   c) amino acid sequences that have 3, 2, or 1 amino acid difference with the amino acid sequence of SEQ ID NO: 2652;
and
CDR3 (AbM definition) has an amino acid sequence selected from:
   a) the amino acid sequence of SEQ ID NOs: 2804;
   b) amino acid sequences that have at least 80% amino acid identity with the amino acid sequence of SEQ ID NO: 2804;
   c) amino acid sequences that have 3, 2, or 1 amino acid difference with the amino acid sequence of SEQ ID NO: 2804.

In one embodiment, the ISVD comprise CDRs (Abm definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 3395, 3376, 3447, 3446, 3643, 2859, and 3653.

In some embodiments, the ISVD may further comprises R45 (Kabat numbering). In some embodiments, the ISVD may further comprises F37 (Kabat numbering). In some embodiments, the ISVD may further comprise F47 (Kabat numbering). In some embodiments, the ISVD may further comprise R45 and F37 (Kabat numbering). In some embodiments, the ISVD may further comprise R45 and F47 (Kabat numbering). In some embodiments, the ISVD may further comprise R45, F37 and F47 (Kabat numbering).In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2453, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2456, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2559, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 3810, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2455, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2556, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2560, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2557, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2450, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2561, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2454, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2558, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2458, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2460, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2553, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2459, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2554, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2464, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2466, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2462, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2465, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2457, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2463, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2461, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2470, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2549, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2550, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2471, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2468, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2472, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2555, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2467, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2469, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2473, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2542, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2547, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2476, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2543, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2480, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2544, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2540, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2477, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2475, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2548, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2545, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2538, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2474, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2546, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2541, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2535, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2486, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2482, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2484, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2537, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2483, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2481, a CDR2 (AbM numbering) that is the amino acid sequence of SEQ ID NO: 2451, and a CDR3 (AbM numbering) that is the amino acid sequence of SEQ ID NO: 2815.

In one embodiment, the ISVD comprises a CDR1 (AbM numbering) that is the amino acid sequence of SEQ ID NO: 2536, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2478, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2479, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2539, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2481, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2487, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2485, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2534, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2528, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2490, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2489, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2529, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2530, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2531, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2492, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2533, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2532, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2488, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2494, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2525, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2491, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2495, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2526, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2524, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2527, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2493, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2521, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2496, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2497, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2512, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2511, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2516, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2515, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2520, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2605, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2522, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2517, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2518, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2514, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2519, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2523, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2499, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2502, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2503, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2504, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2500, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2505, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2509, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2508, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2510, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2501, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2513, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2507, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2506, a CDR2 (AbM numbering) that is the amino acid sequence of SEQ ID NO: 2451, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2698, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2569, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2699, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2564, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2562, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2705, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2695, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2697, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2566, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2700, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2563, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2706, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2565, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2570, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2572, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2701, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2571, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2619, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2575, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2704, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2694, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2702, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2568, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2703, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2533, a CDR2 (AbM numbering) that is the amino acid sequence of SEQ ID NO: 2674, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2596, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2577, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2587, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2583, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2693, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2580, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2579, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2581, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2688, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2578, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2574, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2573, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2582, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2585, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2586, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2593, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2598, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2589, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2588, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2689, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2594, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2600, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2590, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2609, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2591, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2601, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2597, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2595, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2592, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2603, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2602, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2681, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2686, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2690, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2685, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2687, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2683, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2682, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2604, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2599, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2684, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2610, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2678, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2606, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2652, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2651, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2677, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2679, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2672, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2613, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2611, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2676, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2680, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2669, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2509, a CDR2 (AbM numbering) that is the amino acid sequence of SEQ ID NO: 2612, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2614, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2671, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2663, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2664, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2668, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2615, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2673, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2670, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2667, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2616, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2675, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2617, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2661, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2659, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2658, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2657, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2666, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2665, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2625, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2654, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2662, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2622, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2618, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2620, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2621, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2660, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2656, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2634, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2626, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2631, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2632, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2633, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2627, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2628, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2623, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2624, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2655, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2630, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2653, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2629, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2642, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2643, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2647, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2636, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2635, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2637, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2648, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2644, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2638, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2639, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2645, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2646, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2641, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2640, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2649, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2742.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2850.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2707.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2708.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2849.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2848.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2856.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2847.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2852.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2845.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2853.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2854.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2855.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2846.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2851.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2710.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2709.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2844.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2711.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2715.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2712.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2716.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2714.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2718.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2720.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2584, and a CDR3 (AbM numbering) that is the amino acid sequence of SEQ ID NO: 2844.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2719.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2843.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2713.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2717.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2722.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2725.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2723.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2732.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2728.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2726.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2731.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2721.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2729.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2730.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2734.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2727.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2724.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2733.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2833.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2831.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2830.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2839.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2841.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2838.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2834.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2738.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2735.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2832.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2842.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2840.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2739.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2736.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2737.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2771.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2769.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2821.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2775.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2765.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2762.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2768.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2774.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2770.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2767.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2766.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2772.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2773.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2763.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2764.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2779.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2786.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2671, and a CDR3 (AbM numbering) that is the amino acid sequence of SEQ ID NO: 2785.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2783.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2789.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2787.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2777.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2782.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2778.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2784.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2785.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2781.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2790.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2776.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2780.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2788.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2791.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2817.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2814.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2813.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2818.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2809.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2812.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2810.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2820.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2816.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2819.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2808.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2811.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2793.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2806.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2797.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2792.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2800.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2794.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2805.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2807.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2799.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2795.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2798.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2802.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2803.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2796.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2801.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2747.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2826.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2748.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2746.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2749.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2825.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2750.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2743.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2751.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2824.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2745.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2744.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2822.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2757.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2759.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2755.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2756.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2754.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2823.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2760.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2761.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2752.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2758.

In one embodiment, the ISVD comprises a CDR1 (AbM numbering) that is the amino acid sequence of SEQ ID NO: 2498, a CDR2 (AbM numbering) that is the amino acid sequence of SEQ ID NO: 2451, and a CDR3 (AbM numbering) that is the amino acid sequence of SEQ ID NO: 2753.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2830.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2830.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2830.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2830.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2838.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2838.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2838.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2838.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2828.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2828.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2828.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2828.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2836.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2836.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2836.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2836.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2785.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2785.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2785.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2785.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2829.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2829.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2829.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2829.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2837.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2837.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2837.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2837.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2740.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2740.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2740.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2740.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2827.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2827.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2827.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2827.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2651, and a CDR3 (AbM numbering) that is the amino acid sequence of SEQ ID NO: 2835.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2651, and a CDR3 (AbM numbering) that is the amino acid sequence of SEQ ID NO: 2835.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2652, and a CDR3 (AbM numbering) that is the amino acid sequence of SEQ ID NO: 2835.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2652, and a CDR3 (AbM numbering) that is the amino acid sequence of SEQ ID NO: 2835.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2608, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2576, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2691, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2691, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2567, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2576, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2696, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2567, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2696, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2678, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2552, a CDR2 (AbM numbering) that is the amino acid sequence of SEQ ID NO: 2677, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2678, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2677, and a CDR3 (AbM numbering) that is the amino acid sequence of SEQ ID NO: 2741.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2678, and a CDR3 (AbM numbering) that is the amino acid sequence of SEQ ID NO: 2804.

In one embodiment, the ISVD comprises a CDR1 (AbM numbering) that is the amino acid sequence of SEQ ID NO: 2551, a CDR2 (AbM numbering) that is the amino acid sequence of SEQ ID NO: 2677, and a CDR3 (AbM numbering) that is the amino acid sequence of SEQ ID NO: 2804.

In some embodiments, the ISVD may further comprises R45 (Kabat numbering). In some embodiments, the ISVD may further comprises F37 (Kabat numbering). In some embodiments, the ISVD may further comprise F47 (Kabat numbering). In some embodiments, the ISVD may further comprise R45 and F37 (Kabat numbering). In some embodiments, the ISVD may further comprise R45 and F47 (Kabat numbering). In some embodiments, the ISVD may further comprise R45, F37 and F47 (Kabat numbering).

In some embodiments, the amino acid sequence has 80% amino acid sequence identity with one of the amino acid sequences of SEQ ID NOs: 2857-3662, in which for the purposes of determining the degree of amino acid identity, the amino acid residues that form the CDR sequences are disregarded; and in which preferably one or more of the amino acid residues at positions 11, 37, 44, 45, 47, 83, 84, 103, 104 and 108 according to the Kabat numbering are chosen from the Hallmark residues mentioned in Table 1.

In some embodiments, the ISVD essentially consists of a heavy chain variable domain sequence that is derived from a conventional four-chain antibody or that essentially consist of a heavy chain variable domain sequence that is derived from heavy chain antibody.

In some embodiments, the ISVD essentially consists of a VHH, a humanized VHH, a camelized VH, a domain antibody, a single domain antibody, or a dAb.

In some embodiments, the ISVD is a humanized ISVD that is chosen from SEQ ID NOs: 2857-3394 and 3396-3662 or from the group consisting of amino acid sequences that have more than 80%, preferably more than 90%, more preferably more than 95%, such as 99% or more amino acid sequence identity with at least one of the amino acid sequences of SEQ ID NOs: 2857-3662.

In some embodiments, the amino acid sequence is chosen from the group consisting of SEQ ID NO: 3395 and SEQ ID NOs: 2857-3394, 3396-3662.

In some embodiments, the ISVD specifically binds to human PD-1 with a dissociation constant (K_{D}) of 5×10⁻⁹ to 10⁻¹² moles/litre or less, and preferably 10⁻⁹ to 10⁻¹² moles/litre or less and more preferably 10⁻⁹ to 5×10⁻¹¹ moles/litre, such as 7.5×10⁻¹⁰ moles/litre, as determined by Surface Plasmon Resonance e.g., measured at 25° C.

In some embodiments, the ISVD specifically binds to human PD-1 with a kₒₙ-rate of between 10⁴ M⁻¹ s⁻¹ to about 10⁷ M⁻¹s⁻¹, preferably between 5×10⁴ M⁻¹s⁻¹ and 10⁷ M⁻¹s⁻¹, more preferably between 5×10⁴ M⁻¹s⁻¹ and 5×10⁶ M⁻¹s⁻¹, such as between 5×10⁴ M⁻¹s⁻¹ and 1×10⁶ M⁻¹s⁻¹, such as 8.8×10⁴ M⁻¹s⁻¹, as determined by Surface Plasmon Resonance e.g., measured at 25° C.

In some embodiments, the ISVD specifically binds to human PD-1 with a k_{off} rate between 10⁻³ s⁻¹ (t1/2=0.69 s) and 10⁻⁸ s⁻¹ (providing a near irreversible complex with a t1/2 of multiple days), preferably between 10⁻³ s⁻¹ and 5×10⁻⁷ s⁻¹, more preferably between 5×10⁻⁴ s⁻¹ and 5×10⁻⁶ s⁻¹, such as between 10⁻⁴ s⁻¹ and 10⁻⁵ s⁻¹, such as 6.6×10⁻⁵ s⁻¹, as determined by Surface Plasmon Resonance e.g., measured at 25° C.

In some embodiments, the ISVD specifically binds to human and cyno PD-1 and does not bind to CD28, CTLA-4, ICOS, and BTLA or other members of the CD28 receptor family.

In some embodiments, the ISVD antagonizes an activity of PD-L1.

In some embodiments, the ISVD blocks the interaction of human PD-1 with human PD-L1 with a potency (EC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 5×10⁻⁹ M, between 10⁻¹⁰ M and 10⁻⁸ M or between 10⁻⁹ M and 10⁻⁸ M, such as 5.94×10⁻⁹ M, for example, as measured in a FACS competition assay on CHO cells expressing human PD-1 with hPD-L1 as a competitor.

In some embodiments, the ISVD blocks the interaction of cyno PD-1 with cyno PD-L1 with a potency (EC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 5×10⁻⁹ M, between 10⁻¹⁰ M and 10⁻⁸ M or between 10⁻⁹ M and 10⁻⁸ M, such as 4.7×10⁻⁹ M, for example, as measured in a FACS competition assay on CHO cells expressing cyno PD-1 with hPD-L1 as a competitor.

In some embodiments, the ISVD blocks the binding of hPD-1 to hPD-L1 by at least 50%, such as at least 60%, 70%, 80%, 90%, 95%, 99% or even more, as determined by ligand competition, AlphaScreen, or competitive binding assays (such as competition ELISA or competition FACS).

In some embodiments, the ISVD induces T-cell activation with a potency (EC50 value) of 10⁻⁷ M or lower, more preferably of 10⁻⁸ M or lower, or even of 5·10⁻⁹ M or lower, such as between 10⁻¹¹ M and 10⁻⁷ M, between 10⁻¹⁰ M and 5×10⁻⁸ M, between 10⁻¹⁰ M and 10⁻⁸ M or between 10⁻¹¹ M and 10⁻⁹ M, such as 2.69×10⁻⁸ M, or 3.37×10⁻⁸ M, as determined in a TT assay.

Specific examples of such ISVDs that specifically bind to PD-1 have one or more, or all, framework regions as indicated for A0294037F03 and sequence optimized variants thereof in Table A-4 and A-9 (in addition to the CDRs as defined above). In one embodiment, the ISVD comprises or consists of the full amino acid sequence of A0294037F03 (SEQ ID NO: 3395), A029400047 (SEQ ID NO: 2857), A029400048 (SEQ ID NO: 2858), A029400072 (SEQ ID NO: 2859), A029400074 (SEQ ID NO: 2860), A029400104 (SEQ ID NO: 2861), A029400105 (SEQ ID NO: 2862), A029400106 (SEQ ID NO: 2863), A029400107 (SEQ ID NO: 2864), A029400108 (SEQ ID NO: 2865), A029400109 (SEQ ID NO: 2866), A029400110 (SEQ ID NO: 2867), A029400111 (SEQ ID NO: 2868), A029400161 (SEQ ID NO: 2869), A029400169 (SEQ ID NO: 2870), A029400170 (SEQ ID NO: 2871), A029400171 (SEQ ID NO: 2872), A029400172 (SEQ ID NO: 2873), A029400173 (SEQ ID NO: 2874), A029400174 (SEQ ID NO: 2875), A029400175 (SEQ ID NO: 2876), A029400176 (SEQ ID NO: 2877), A029400177 (SEQ ID NO: 2878), A029400178 (SEQ ID NO: 2879), A029400205 (SEQ ID NO: 2880), A029400206 (SEQ ID NO: 2881), A029400207 (SEQ ID NO: 2882), A029400208 (SEQ ID NO: 2883), A029400210 (SEQ ID NO: 2884), A029400212 (SEQ ID NO: 2885), A029400214 (SEQ ID NO: 2886), A029400217 (SEQ ID NO: 2887), A029400221 (SEQ ID NO: 2888), A029400222 (SEQ ID NO: 2889), A029400228 (SEQ ID NO: 2890), A029400236 (SEQ ID NO: 2891), A029400244 (SEQ ID NO: 2892), A029400245 (SEQ ID NO: 2893), A029400246 (SEQ ID NO: 2894), A029400248 (SEQ ID NO: 2895), A029400249 (SEQ ID NO: 2896), A029400250 (SEQ ID NO: 2897), A029400253 (SEQ ID NO: 2898), A029400254 (SEQ ID NO: 2899), A029400256 (SEQ ID NO: 2900), A029400262 (SEQ ID NO: 2901), A029400268 (SEQ ID NO: 2902), A029400276 (SEQ ID NO: 2903), A029400281 (SEQ ID NO: 2904), A029400282 (SEQ ID NO: 2905), A029400283 (SEQ ID NO: 2906), A029400284 (SEQ ID NO: 2907), A029400285 (SEQ ID NO: 2908), A029400286 (SEQ ID NO: 2909), A029400287 (SEQ ID NO: 2910), A029400288 (SEQ ID NO: 2911), A029400289 (SEQ ID NO: 2912), A029400294 (SEQ ID NO: 2913), A029400296 (SEQ ID NO: 2914), A029400306 (SEQ ID NO: 2915), A029400319 (SEQ ID NO: 2916), A029400320 (SEQ ID NO: 2917), A029400321 (SEQ ID NO: 2918), A029400322 (SEQ ID NO: 2919), A029400323 (SEQ ID NO: 2920), A029400324 (SEQ ID NO: 2921), A029400325 (SEQ ID NO: 2922), A029400326 (SEQ ID NO: 2923), A029400327 (SEQ ID NO: 2924), A029400332 (SEQ ID NO: 2925), A029400335 (SEQ ID NO: 2926), A029400336 (SEQ ID NO: 2927), A029400345 (SEQ ID NO: 2928), A029400349 (SEQ ID NO: 2929), A029400353 (SEQ ID NO: 2930), A029400357 (SEQ ID NO: 2931), A029400358 (SEQ ID NO: 2932), A029400359 (SEQ ID NO: 2933), A029400360 (SEQ ID NO: 2934), A029400362 (SEQ ID NO: 2935), A029400363 (SEQ ID NO: 2936), A029400365 (SEQ ID NO: 2937), A029400366 (SEQ ID NO: 2938), A029400367 (SEQ ID NO: 2939), A029400368 (SEQ ID NO: 2940), A029400371 (SEQ ID NO: 2941), A029400382 (SEQ ID NO: 2942), A029400383 (SEQ ID NO: 2943), A029400384 (SEQ ID NO: 2944), A029400392 (SEQ ID NO: 2945), A029400395 (SEQ ID NO: 2946), A029400398 (SEQ ID NO: 2947), A029400401 (SEQ ID NO: 2948), A029400402 (SEQ ID NO: 2949), A029400406 (SEQ ID NO: 2950), A029400407 (SEQ ID NO: 2951), A029400414 (SEQ ID NO: 2952), A029400417 (SEQ ID NO: 2953), A029400419 (SEQ ID NO: 2954), A029400421 (SEQ ID NO: 2955), A029400428 (SEQ ID NO: 2956), A029400430 (SEQ ID NO: 2957), A029400431 (SEQ ID NO: 2958), A029400432 (SEQ ID NO: 2959), A029400437 (SEQ ID NO: 2960), A029400438 (SEQ ID NO: 2961), A029400439 (SEQ ID NO: 2962), A029400441 (SEQ ID NO: 2963), A029400442 (SEQ ID NO: 2964), A029400444 (SEQ ID NO: 2965), A029400451 (SEQ ID NO: 2966), A029400461 (SEQ ID NO: 2967), A029400462 (SEQ ID NO: 2968), A029400463 (SEQ ID NO: 2969), A029400464 (SEQ ID NO: 2970), A029400466 (SEQ ID NO: 2971), A029400470 (SEQ ID NO: 2972), A029400471 (SEQ ID NO: 2973), A029400472 (SEQ ID NO: 2974), A029400475 (SEQ ID NO: 2975), A029400477 (SEQ ID NO: 2976), A029400484 (SEQ ID NO: 2977), A029400489 (SEQ ID NO: 2978), A029400491 (SEQ ID NO: 2979), A029400492 (SEQ ID NO: 2980), A029400493 (SEQ ID NO: 2981), A029400494 (SEQ ID NO: 2982), A029400495 (SEQ ID NO: 2983), A029400497 (SEQ ID NO: 2984), A029400498 (SEQ ID NO: 2985), A029400499 (SEQ ID NO: 2986), A029400501 (SEQ ID NO: 2987), A029400502 (SEQ ID NO: 2988), A029400504 (SEQ ID NO: 2989), A029400509 (SEQ ID NO: 2990), A029400511 (SEQ ID NO: 2991), A029400515 (SEQ ID NO: 2992), A029400516 (SEQ ID NO: 2993), A029400529 (SEQ ID NO: 2994), A029400530 (SEQ ID NO: 2995), A029400531 (SEQ ID NO: 2996), A029400532 (SEQ ID NO: 2997), A029400533 (SEQ ID NO: 2998), A029400534 (SEQ ID NO: 2999), A029400535 (SEQ ID NO: 3000), A029400536 (SEQ ID NO: 3001), A029400537 (SEQ ID NO: 3002), A029400538 (SEQ ID NO: 3003), A029400539 (SEQ ID NO: 3004), A029400541 (SEQ ID NO: 3005), A029400542 (SEQ ID NO: 3006), A029400547 (SEQ ID NO: 3007), A029400552 (SEQ ID NO: 3008), A029400571 (SEQ ID NO: 3009), A029400572 (SEQ ID NO: 3010), A029400573 (SEQ ID NO: 3011), A029400574 (SEQ ID NO: 3012), A029400575 (SEQ ID NO: 3013), A029400576 (SEQ ID NO: 3014), A029400579 (SEQ ID NO: 3015), A029400580 (SEQ ID NO: 3016), A029400581 (SEQ ID NO: 3017), A029400582 (SEQ ID NO: 3018), A029400592 (SEQ ID NO: 3019), A029400601 (SEQ ID NO: 3020), A029400603 (SEQ ID NO: 3021), A029400605 (SEQ ID NO: 3022), A029400606 (SEQ ID NO: 3023), A029400607 (SEQ ID NO: 3024), A029400608 (SEQ ID NO: 3025), A029400610 (SEQ ID NO: 3026), A029400612 (SEQ ID NO: 3027), A029400614 (SEQ ID NO: 3028), A029400616 (SEQ ID NO: 3029), A029400617 (SEQ ID NO: 3030), A029400619 (SEQ ID NO: 3031), A029400621 (SEQ ID NO: 3032), A029400625 (SEQ ID NO: 3033), A029400627 (SEQ ID NO: 3034), A029400632 (SEQ ID NO: 3035), A029400642 (SEQ ID NO: 3036), A029400643 (SEQ ID NO: 3037), A029400644 (SEQ ID NO: 3038), A029400645 (SEQ ID NO: 3039), A029400646 (SEQ ID NO: 3040), A029400648 (SEQ ID NO: 3041), A029400649 (SEQ ID NO: 3042), A029400650 (SEQ ID NO: 3043), A029400651 (SEQ ID NO: 3044), A029400658 (SEQ ID NO: 3045), A029400659 (SEQ ID NO: 3046), A029400660 (SEQ ID NO: 3047), A029400664 (SEQ ID NO: 3048), A029400665 (SEQ ID NO: 3049), A029400666 (SEQ ID NO: 3050), A029400671 (SEQ ID NO: 3051), A029400679 (SEQ ID NO: 3052), A029400680 (SEQ ID NO: 3053), A029400681 (SEQ ID NO: 3054), A029400682 (SEQ ID NO: 3055), A029400686 (SEQ ID NO: 3056), A029400689 (SEQ ID NO: 3057), A029400690 (SEQ ID NO: 3058), A029400691 (SEQ ID NO: 3059), A029400694 (SEQ ID NO: 3060), A029400696 (SEQ ID NO: 3061), A029400707 (SEQ ID NO: 3062), A029400708 (SEQ ID NO: 3063), A029400709 (SEQ ID NO: 3064), A029400711 (SEQ ID NO: 3065), A029400713 (SEQ ID NO: 3066), A029400715 (SEQ ID NO: 3067), A029400716 (SEQ ID NO: 3068), A029400717 (SEQ ID NO: 3069), A029400718 (SEQ ID NO: 3070), A029400719 (SEQ ID NO: 3071), A029400722 (SEQ ID NO: 3072), A029400723 (SEQ ID NO: 3073), A029400731 (SEQ ID NO: 3074), A029400732 (SEQ ID NO: 3075), A029400738 (SEQ ID NO: 3076), A029400739 (SEQ ID NO: 3077), A029400740 (SEQ ID NO: 3078), A029400741 (SEQ ID NO: 3079), A029400742 (SEQ ID NO: 3080), A029400743 (SEQ ID NO: 3081), A029400747 (SEQ ID NO: 3082), A029400749 (SEQ ID NO: 3083), A029400758 (SEQ ID NO: 3084), A029400761 (SEQ ID NO: 3085), A029400762 (SEQ ID NO: 3086), A029400765 (SEQ ID NO: 3087), A029400776 (SEQ ID NO: 3088), A029400777 (SEQ ID NO: 3089), A029400778 (SEQ ID NO: 3090), A029400779 (SEQ ID NO: 3091), A029400780 (SEQ ID NO: 3092), A029400781 (SEQ ID NO: 3093), A029400782 (SEQ ID NO: 3094), A029400783 (SEQ ID NO: 3095), A029400785 (SEQ ID NO: 3096), A029400787 (SEQ ID NO: 3097), A029400791 (SEQ ID NO: 3098), A029400792 (SEQ ID NO: 3099), A029400793 (SEQ ID NO: 3100), A029400795 (SEQ ID NO: 3101), A029400798 (SEQ ID NO: 3102), A029400800 (SEQ ID NO: 3103), A029400814 (SEQ ID NO: 3104), A029400815 (SEQ ID NO: 3105), A029400816 (SEQ ID NO: 3106), A029400817 (SEQ ID NO: 3107), A029400818 (SEQ ID NO: 3108), A029400819 (SEQ ID NO: 3109), A029400820 (SEQ ID NO: 3110), A029400822 (SEQ ID NO: 3111), A029400823 (SEQ ID NO: 3112), A029400824 (SEQ ID NO: 3113), A029400828 (SEQ ID NO: 3114), A029400837 (SEQ ID NO: 3115), A029400838 (SEQ ID NO: 3116), A029400846 (SEQ ID NO: 3117), A029400847 (SEQ ID NO: 3118), A029400848 (SEQ ID NO: 3119), A029400849 (SEQ ID NO: 3120), A029400850 (SEQ ID NO: 3121), A029400851 (SEQ ID NO: 3122), A029400852 (SEQ ID NO: 3123), A029400853 (SEQ ID NO: 3124), A029400855 (SEQ ID NO: 3125), A029400856 (SEQ ID NO: 3126), A029400859 (SEQ ID NO: 3127), A029400861 (SEQ ID NO: 3128), A029400868 (SEQ ID NO: 3129), A029400871 (SEQ ID NO: 3130), A029400873 (SEQ ID NO: 3131), A029400878 (SEQ ID NO: 3132), A029400879 (SEQ ID NO: 3133), A029400880 (SEQ ID NO: 3134), A029400881 (SEQ ID NO: 3135), A029400882 (SEQ ID NO: 3136), A029400885 (SEQ ID NO: 3137), A029400887 (SEQ ID NO: 3138), A029400890 (SEQ ID NO: 3139), A029400891 (SEQ ID NO: 3140), A029400892 (SEQ ID NO: 3141), A029400894 (SEQ ID NO: 3142), A029400908 (SEQ ID NO: 3143), A029400911 (SEQ ID NO: 3144), A029400912 (SEQ ID NO: 3145), A029400913 (SEQ ID NO: 3146), A029400914 (SEQ ID NO: 3147), A029400915 (SEQ ID NO: 3148), A029400916 (SEQ ID NO: 3149), A029400917 (SEQ ID NO: 3150), A029400919 (SEQ ID NO: 3151), A029400922 (SEQ ID NO: 3152), A029400927 (SEQ ID NO: 3153), A029400930 (SEQ ID NO: 3154), A029400931 (SEQ ID NO: 3155), A029400932 (SEQ ID NO: 3156), A029400940 (SEQ ID NO: 3157), A029400941 (SEQ ID NO: 3158), A029400942 (SEQ ID NO: 3159), A029400943 (SEQ ID NO: 3160), A029400944 (SEQ ID NO: 3161), A029400945 (SEQ ID NO: 3162), A029400946 (SEQ ID NO: 3163), A029400948 (SEQ ID NO: 3164), A029400949 (SEQ ID NO: 3165), A029400950 (SEQ ID NO: 3166), A029400951 (SEQ ID NO: 3167), A029400954 (SEQ ID NO: 3168), A029400956 (SEQ ID NO: 3169), A029400960 (SEQ ID NO: 3170), A029400969 (SEQ ID NO: 3171), A029400970 (SEQ ID NO: 3172), A029400972 (SEQ ID NO: 3173), A029400973 (SEQ ID NO: 3174), A029400974 (SEQ ID NO: 3175), A029400975 (SEQ ID NO: 3176), A029400976 (SEQ ID NO: 3177), A029400979 (SEQ ID NO: 3178), A029400984 (SEQ ID NO: 3179), A029400985 (SEQ ID NO: 3180), A029400986 (SEQ ID NO: 3181), A029400988 (SEQ ID NO: 3182), A029400992 (SEQ ID NO: 3183), A029401006 (SEQ ID NO: 3184), A029401007 (SEQ ID NO: 3185), A029401009 (SEQ ID NO: 3186), A029401010 (SEQ ID NO: 3187), A029401012 (SEQ ID NO: 3188), A029401013 (SEQ ID NO: 3189), A029401016 (SEQ ID NO: 3190), A029401017 (SEQ ID NO: 3191), A029401018 (SEQ ID NO: 3192), A029401019 (SEQ ID NO: 3193), A029401021 (SEQ ID NO: 3194), A029401028 (SEQ ID NO: 3195), A029401036 (SEQ ID NO: 3196), A029401037 (SEQ ID NO: 3197), A029401041 (SEQ ID NO: 3198), A029401042 (SEQ ID NO: 3199), A029401043 (SEQ ID NO: 3200), A029401045 (SEQ ID NO: 3201), A029401046 (SEQ ID NO: 3202), A029401047 (SEQ ID NO: 3203), A029401051 (SEQ ID NO: 3204), A029401052 (SEQ ID NO: 3205), A029401053 (SEQ ID NO: 3206), A029401054 (SEQ ID NO: 3207), A029401057 (SEQ ID NO: 3208), A029401060 (SEQ ID NO: 3209), A029401063 (SEQ ID NO: 3210), A029401064 (SEQ ID NO: 3211), A029401065 (SEQ ID NO: 3212), A029401068 (SEQ ID NO: 3213), A029401069 (SEQ ID NO: 3214), A029401070 (SEQ ID NO: 3215), A029401071 (SEQ ID NO: 3216), A029401074 (SEQ ID NO: 3217), A029401075 (SEQ ID NO: 3218), A029401076 (SEQ ID NO: 3219), A029401077 (SEQ ID NO: 3220), A029401080 (SEQ ID NO: 3221), A029401081 (SEQ ID NO: 3222), A029401082 (SEQ ID NO: 3223), A029401084 (SEQ ID NO: 3224), A029401089 (SEQ ID NO: 3225), A029401092 (SEQ ID NO: 3226), A029401097 (SEQ ID NO: 3227), A029401098 (SEQ ID NO: 3228), A029401099 (SEQ ID NO: 3229), A029401100 (SEQ ID NO: 3230), A029401101 (SEQ ID NO: 3231), A029401103 (SEQ ID NO: 3232), A029401104 (SEQ ID NO: 3233), A029401105 (SEQ ID NO: 3234), A029401107 (SEQ ID NO: 3235), A029401108 (SEQ ID NO: 3236), A029401109 (SEQ ID NO: 3237), A029401110 (SEQ ID NO: 3238), A029401118 (SEQ ID NO: 3239), A029401121 (SEQ ID NO: 3240), A029401129 (SEQ ID NO: 3241), A029401130 (SEQ ID NO: 3242), A029401131 (SEQ ID NO: 3243), A029401132 (SEQ ID NO: 3244), A029401133 (SEQ ID NO: 3245), A029401134 (SEQ ID NO: 3246), A029401135 (SEQ ID NO: 3247), A029401136 (SEQ ID NO: 3248), A029401137 (SEQ ID NO: 3249), A029401138 (SEQ ID NO: 3250), A029401141 (SEQ ID NO: 3251), A029401143 (SEQ ID NO: 3252), A029401145 (SEQ ID NO: 3253), A029401146 (SEQ ID NO: 3254), A029401150 (SEQ ID NO: 3255), A029401152 (SEQ ID NO: 3256), A029401165 (SEQ ID NO: 3257), A029401166 (SEQ ID NO: 3258), A029401167 (SEQ ID NO: 3259), A029401168 (SEQ ID NO: 3260), A029401169 (SEQ ID NO: 3261), A029401170 (SEQ ID NO: 3262), A029401172 (SEQ ID NO: 3263), A029401173 (SEQ ID NO: 3264), A029401174 (SEQ ID NO: 3265), A029401175 (SEQ ID NO: 3266), A029401177 (SEQ ID NO: 3267), A029401178 (SEQ ID NO: 3268), A029401179 (SEQ ID NO: 3269), A029401187 (SEQ ID NO: 3270), A029401190 (SEQ ID NO: 3271), A029401202 (SEQ ID NO: 3272), A029401203 (SEQ ID NO: 3273), A029401204 (SEQ ID NO: 3274), A029401205 (SEQ ID NO: 3275), A029401206 (SEQ ID NO: 3276), A029401209 (SEQ ID NO: 3277), A029401210 (SEQ ID NO: 3278), A029401211 (SEQ ID NO: 3279), A029401212 (SEQ ID NO: 3280), A029401213 (SEQ ID NO: 3281), A029401214 (SEQ ID NO: 3282), A029401215 (SEQ ID NO: 3283), A029401219 (SEQ ID NO: 3284), A029401222 (SEQ ID NO: 3285), A029401224 (SEQ ID NO: 3286), A029401240 (SEQ ID NO: 3287), A029401241 (SEQ ID NO: 3288), A029401242 (SEQ ID NO: 3289), A029401243 (SEQ ID NO: 3290), A029401244 (SEQ ID NO: 3291), A029401247 (SEQ ID NO: 3292), A029401248 (SEQ ID NO: 3293), A029401249 (SEQ ID NO: 3294), A029401250 (SEQ ID NO: 3295), A029401251 (SEQ ID NO: 3296), A029401252 (SEQ ID NO: 3297), A029401254 (SEQ ID NO: 3298), A029401257 (SEQ ID NO: 3299), A029401259 (SEQ ID NO: 3300), A029401267 (SEQ ID NO: 3301), A029401273 (SEQ ID NO: 3302), A029401275 (SEQ ID NO: 3303), A029401276 (SEQ ID NO: 3304), A029401278 (SEQ ID NO: 3305), A029401280 (SEQ ID NO: 3306), A029401281 (SEQ ID NO: 3307), A029401282 (SEQ ID NO: 3308), A029401283 (SEQ ID NO: 3309), A029401284 (SEQ ID NO: 3310), A029401286 (SEQ ID NO: 3311), A029401289 (SEQ ID NO: 3312), A029401290 (SEQ ID NO: 3313), A029401292 (SEQ ID NO: 3314), A029401296 (SEQ ID NO: 3315), A029401298 (SEQ ID NO: 3316), A029401303 (SEQ ID NO: 3317), A029401308 (SEQ ID NO: 3318), A029401309 (SEQ ID NO: 3319), A029401310 (SEQ ID NO: 3320), A029401311 (SEQ ID NO: 3321), A029401312 (SEQ ID NO: 3322), A029401313 (SEQ ID NO: 3323), A029401314 (SEQ ID NO: 3324), A029401315 (SEQ ID NO: 3325), A029401316 (SEQ ID NO: 3326), A029401320 (SEQ ID NO: 3327), A029401327 (SEQ ID NO: 3328), A029401330 (SEQ ID NO: 3329), A029401336 (SEQ ID NO: 3330), A029401337 (SEQ ID NO: 3331), A029401341 (SEQ ID NO: 3332), A029401342 (SEQ ID NO: 3333), A029401343 (SEQ ID NO: 3334), A029401344 (SEQ ID NO: 3335), A029401345 (SEQ ID NO: 3336), A029401346 (SEQ ID NO: 3337), A029401347 (SEQ ID NO: 3338), A029401348 (SEQ ID NO: 3339), A029401350 (SEQ ID NO: 3340), A029401352 (SEQ ID NO: 3341), A029401354 (SEQ ID NO: 3342), A029401357 (SEQ ID NO: 3343), A029401367 (SEQ ID NO: 3344), A029401368 (SEQ ID NO: 3345), A029401372 (SEQ ID NO: 3346), A029401374 (SEQ ID NO: 3347), A029401377 (SEQ ID NO: 3348), A029401378 (SEQ ID NO: 3349), A029401379 (SEQ ID NO: 3350), A029401380 (SEQ ID NO: 3351), A029401381 (SEQ ID NO: 3352), A029401382 (SEQ ID NO: 3353), A029401383 (SEQ ID NO: 3354), A029401385 (SEQ ID NO: 3355), A029401386 (SEQ ID NO: 3356), A029401391 (SEQ ID NO: 3357), A029401392 (SEQ ID NO: 3358), A029401393 (SEQ ID NO: 3359), A029401398 (SEQ ID NO: 3360), A029401408 (SEQ ID NO: 3361), A029401411 (SEQ ID NO: 3362), A029401415 (SEQ ID NO: 3363), A029401416 (SEQ ID NO: 3364), A029401417 (SEQ ID NO: 3365), A029401418 (SEQ ID NO: 3366), A029401419 (SEQ ID NO: 3367), A029401420 (SEQ ID NO: 3368), A029401422 (SEQ ID NO: 3369), A029401423 (SEQ ID NO: 3370), A029401424 (SEQ ID NO: 3371), A029401428 (SEQ ID NO: 3372), A029401430 (SEQ ID NO: 3373), A029401437 (SEQ ID NO: 3374), A029401440 (SEQ ID NO: 3375), A029403741 (SEQ ID NO: 3376), A029403742 (SEQ ID NO: 3377), A029403743 (SEQ ID NO: 3378), A029403744 (SEQ ID NO: 3379), A029403750 (SEQ ID NO: 3380), A029403751 (SEQ ID NO: 3381), A029403752 (SEQ ID NO: 3382), A029403753 (SEQ ID NO: 3383), A029403754 (SEQ ID NO: 3384), A029403755 (SEQ ID NO: 3385), A029403756 (SEQ ID NO: 3386), A029403757 (SEQ ID NO: 3387), A029403758 (SEQ ID NO: 3388), A029403759 (SEQ ID NO: 3389), A029403760 (SEQ ID NO: 3390), A029403761 (SEQ ID NO: 3391), A029403762 (SEQ ID NO: 3392), A029403763 (SEQ ID NO: 3393), A029403764 (SEQ ID NO: 3394), A029404010 (SEQ ID NO: 3396), A029404011 (SEQ ID NO: 3397), A029404015 (SEQ ID NO: 3398), A029404016 (SEQ ID NO: 3399), A029404017 (SEQ ID NO: 3400), A029404030 (SEQ ID NO: 3401), A029404032 (SEQ ID NO: 3402), A029404034 (SEQ ID NO: 3403), A029404430 (SEQ ID NO: 3404), A029404431 (SEQ ID NO: 3405), A029404432 (SEQ ID NO: 3406), A029404433 (SEQ ID NO: 3407), A029404434 (SEQ ID NO: 3408), A029404435 (SEQ ID NO: 3409), A029404436 (SEQ ID NO: 3410), A029404437 (SEQ ID NO: 3411), A029404438 (SEQ ID NO: 3412), A029404439 (SEQ ID NO: 3413), A029404440 (SEQ ID NO: 3414), A029404441 (SEQ ID NO: 3415), A029404442 (SEQ ID NO: 3416), A029404443 (SEQ ID NO: 3417), A029404444 (SEQ ID NO: 3418), A029404445 (SEQ ID NO: 3419), A029404446 (SEQ ID NO: 3420), A029404447 (SEQ ID NO: 3421), A029404448 (SEQ ID NO: 3422), A029404449 (SEQ ID NO: 3423), A029404450 (SEQ ID NO: 3424), A029404451 (SEQ ID NO: 3425), A029404452 (SEQ ID NO: 3426), A029404453 (SEQ ID NO: 3427), A029404454 (SEQ ID NO: 3428), A029404455 (SEQ ID NO: 3429), A029404456 (SEQ ID NO: 3430), A029404457 (SEQ ID NO: 3431), A029404458 (SEQ ID NO: 3432), A029404459 (SEQ ID NO: 3433), A029404460 (SEQ ID NO: 3434), A029404461 (SEQ ID NO: 3435), A029404462 (SEQ ID NO: 3436), A029404463 (SEQ ID NO: 3437), A029404464 (SEQ ID NO: 3438), A029404465 (SEQ ID NO: 3439), A029404466 (SEQ ID NO: 3440), A029404467 (SEQ ID NO: 3441), A029404468 (SEQ ID NO: 3442), A029404469 (SEQ ID NO: 3443), A029404470 (SEQ ID NO: 3444), A029404471 (SEQ ID NO: 3445), A029404472 (SEQ ID NO: 3446), A029404473 (SEQ ID NO: 3447), A029404474 (SEQ ID NO: 3448), A029404475 (SEQ ID NO: 3449), A029404476 (SEQ ID NO: 3450), A029404477 (SEQ ID NO: 3451), A029404478 (SEQ ID NO: 3452), A029404479 (SEQ ID NO: 3453), A029404480 (SEQ ID NO: 3454), A029404481 (SEQ ID NO: 3455), A029404482 (SEQ ID NO: 3456), A029404483 (SEQ ID NO: 3457), A029404484 (SEQ ID NO: 3458), A029404485 (SEQ ID NO: 3459), A029404486 (SEQ ID NO: 3460), A029404487 (SEQ ID NO: 3461), A029404488 (SEQ ID NO: 3462), A029404489 (SEQ ID NO: 3463), A029404490 (SEQ ID NO: 3464), A029404491 (SEQ ID NO: 3465), A029404492 (SEQ ID NO: 3466), A029404493 (SEQ ID NO: 3467), A029404494 (SEQ ID NO: 3468), A029404495 (SEQ ID NO: 3469), A029404496 (SEQ ID NO: 3470), A029404497 (SEQ ID NO: 3471), A029404498 (SEQ ID NO: 3472), A029404499 (SEQ ID NO: 3473), A029404500 (SEQ ID NO: 3474), A029404501 (SEQ ID NO: 3475), A029404502 (SEQ ID NO: 3476), A029404503 (SEQ ID NO: 3477), A029404504 (SEQ ID NO: 3478), A029404505 (SEQ ID NO: 3479), A029404506 (SEQ ID NO: 3480), A029404507 (SEQ ID NO: 3481), A029404508 (SEQ ID NO: 3482), A029404509 (SEQ ID NO: 3483), A029404510 (SEQ ID NO: 3484), A029404511 (SEQ ID NO: 3485), A029404512 (SEQ ID NO: 3486), A029404513 (SEQ ID NO: 3487), A029404514 (SEQ ID NO: 3488), A029404515 (SEQ ID NO: 3489), A029404516 (SEQ ID NO: 3490), A029404517 (SEQ ID NO: 3491), A029404518 (SEQ ID NO: 3492), A029404519 (SEQ ID NO: 3493), A029404520 (SEQ ID NO: 3494), A029404521 (SEQ ID NO: 3495), A029404522 (SEQ ID NO: 3496), A029404523 (SEQ ID NO: 3497), A029404524 (SEQ ID NO: 3498), A029404525 (SEQ ID NO: 3499), A029404526 (SEQ ID NO: 3500), A029404527 (SEQ ID NO: 3501), A029404528 (SEQ ID NO: 3502), A029404529 (SEQ ID NO: 3503), A029404530 (SEQ ID NO: 3504), A029404531 (SEQ ID NO: 3505), A029404532 (SEQ ID NO: 3506), A029404533 (SEQ ID NO: 3507), A029404534 (SEQ ID NO: 3508), A029404535 (SEQ ID NO: 3509), A029404536 (SEQ ID NO: 3510), A029404537 (SEQ ID NO: 3511), A029404538 (SEQ ID NO: 3512), A029404539 (SEQ ID NO: 3513), A029404540 (SEQ ID NO: 3514), A029404541 (SEQ ID NO: 3515), A029404542 (SEQ ID NO: 3516), A029404543 (SEQ ID NO: 3517), A029404544 (SEQ ID NO: 3518), A029404545 (SEQ ID NO: 3519), A029404546 (SEQ ID NO: 3520), A029404547 (SEQ ID NO: 3521), A029404548 (SEQ ID NO: 3522), A029404549 (SEQ ID NO: 3523), A029404550 (SEQ ID NO: 3524), A029404551 (SEQ ID NO: 3525), A029404552 (SEQ ID NO: 3526), A029404553 (SEQ ID NO: 3527), A029404554 (SEQ ID NO: 3528), A029404555 (SEQ ID NO: 3529), A029404556 (SEQ ID NO: 3530), A029404557 (SEQ ID NO: 3531), A029404558 (SEQ ID NO: 3532), A029404559 (SEQ ID NO: 3533), A029404560 (SEQ ID NO: 3534), A029404561 (SEQ ID NO: 3535), A029404562 (SEQ ID NO: 3536), A029404563 (SEQ ID NO: 3537), A029404564 (SEQ ID NO: 3538), A029404565 (SEQ ID NO: 3539), A029404566 (SEQ ID NO: 3540), A029404567 (SEQ ID NO: 3541), A029404568 (SEQ ID NO: 3542), A029404569 (SEQ ID NO: 3543), A029404570 (SEQ ID NO: 3544), A029404571 (SEQ ID NO: 3545), A029404572 (SEQ ID NO: 3546), A029404573 (SEQ ID NO: 3547), A029404574 (SEQ ID NO: 3548), A029404575 (SEQ ID NO: 3549), A029404576 (SEQ ID NO: 3550), A029404577 (SEQ ID NO: 3551), A029404578 (SEQ ID NO: 3552), A029404579 (SEQ ID NO: 3553), A029404580 (SEQ ID NO: 3554), A029404581 (SEQ ID NO: 3555), A029404582 (SEQ ID NO: 3556), A029404583 (SEQ ID NO: 3557), A029404584 (SEQ ID NO: 3558), A029404585 (SEQ ID NO: 3559), A029404586 (SEQ ID NO: 3560), A029404587 (SEQ ID NO: 3561), A029404588 (SEQ ID NO: 3562), A029404589 (SEQ ID NO: 3563), A029404590 (SEQ ID NO: 3564), A029404591 (SEQ ID NO: 3565), A029404592 (SEQ ID NO: 3566), A029404593 (SEQ ID NO: 3567), A029404594 (SEQ ID NO: 3568), A029404595 (SEQ ID NO: 3569), A029404596 (SEQ ID NO: 3570), A029404597 (SEQ ID NO: 3571), A029404598 (SEQ ID NO: 3572), A029404599 (SEQ ID NO: 3573), A029404600 (SEQ ID NO: 3574), A029404601 (SEQ ID NO: 3575), A029404603 (SEQ ID NO: 3576), A029404604 (SEQ ID NO: 3577), A029404605 (SEQ ID NO: 3578), A029404606 (SEQ ID NO: 3579), A029404607 (SEQ ID NO: 3580), A029404621 (SEQ ID NO: 3581), A029404622 (SEQ ID NO: 3582), A029404623 (SEQ ID NO: 3583), A029404624 (SEQ ID NO: 3584), A029404625 (SEQ ID NO: 3585), A029404678 (SEQ ID NO: 3586), A029404679 (SEQ ID NO: 3587), A029404680 (SEQ ID NO: 3588), A029404681 (SEQ ID NO: 3589), A029404682 (SEQ ID NO: 3590), A029404683 (SEQ ID NO: 3591), A029404684 (SEQ ID NO: 3592), A029404685 (SEQ ID NO: 3593), A029404749 (SEQ ID NO: 3594), A029404750 (SEQ ID NO: 3595), A029404763 (SEQ ID NO: 3596), A029404764 (SEQ ID NO: 3597), A029404765 (SEQ ID NO: 3598), A029404766 (SEQ ID NO: 3599), A029404767 (SEQ ID NO: 3600), A029404769 (SEQ ID NO: 3601), A029404770 (SEQ ID NO: 3602), A029404771 (SEQ ID NO: 3603), A029404772 (SEQ ID NO: 3604), A029404774 (SEQ ID NO: 3605), A029404775 (SEQ ID NO: 3606), A029404776 (SEQ ID NO: 3607), A029404777 (SEQ ID NO: 3608), A029404779 (SEQ ID NO: 3609), A029404780 (SEQ ID NO: 3610), A029404781 (SEQ ID NO: 3611), A029404782 (SEQ ID NO: 3612), A029404783 (SEQ ID NO: 3613), A029404785 (SEQ ID NO: 3614), A029404786 (SEQ ID NO: 3615), A029404787 (SEQ ID NO: 3616), A029404788 (SEQ ID NO: 3617), A029404789 (SEQ ID NO: 3618), A029404790 (SEQ ID NO: 3619), A029404791 (SEQ ID NO: 3620), A029404792 (SEQ ID NO: 3621), A029404793 (SEQ ID NO: 3622), A029404794 (SEQ ID NO: 3623), A029404795 (SEQ ID NO: 3624), A029404796 (SEQ ID NO: 3625), A029404797 (SEQ ID NO: 3626), A029404798 (SEQ ID NO: 3627), A029404799 (SEQ ID NO: 3628), A029404800 (SEQ ID NO: 3629), A029404801 (SEQ ID NO: 3630), A029404802 (SEQ ID NO: 3631), A029404803 (SEQ ID NO: 3632), A029404805 (SEQ ID NO: 3633), A029404806 (SEQ ID NO: 3634), A029404807 (SEQ ID NO: 3635), A029404808 (SEQ ID NO: 3636), A029405036 (SEQ ID NO: 3637), A029405065 (SEQ ID NO: 3638), A029405066 (SEQ ID NO: 3639), A029405074 (SEQ ID NO: 3640), A029405076 (SEQ ID NO: 3641), A029405077 (SEQ ID NO: 3642), A029405164 (SEQ ID NO: 3643), A029405166 (SEQ ID NO: 3644), A029405167 (SEQ ID NO: 3645), A029405168 (SEQ ID NO: 3646), A029405169 (SEQ ID NO: 3647), A029405170 (SEQ ID NO: 3648), A029405171 (SEQ ID NO: 3649), A029405172 (SEQ ID NO: 3650), A029405173 (SEQ ID NO: 3651), A029405174 (SEQ ID NO: 3652), A029405175 (SEQ ID NO: 3653), A029405177 (SEQ ID NO: 3654), A029405178 (SEQ ID NO: 3655), A029405179 (SEQ ID NO: 3656), A029405180 (SEQ ID NO: 3657), A029405181 (SEQ ID NO: 3658), A029405182 (SEQ ID NO: 3659), A029405183 (SEQ ID NO: 3660), A029405184 (SEQ ID NO: 3661), and A029405185 (SEQ ID NO: 3662) (see Table A-4 and A-9).

The SEQ ID NOs for the CDR sequences referred to above are based on the CDR definition according to the AbM definition (see Table A-4 and A-9). It is noted that the SEQ ID NOs for the CDR sequences defined according to the Kabat definition, Chothia definition or IMGT definition can likewise be used. Accordingly, the ISVDs provided by the present technology, specifically binding to PD-1 as described above using the AbM definition, can be also described using the Kabat definition, Chothia definition or IMGT definition.

In one embodiment, the ISVD comprises CDRs (Kabat definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 2857-3662.

In one embodiment, the ISVD comprises CDRs (Kabat definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 3395, 3376, 3447, 3446, 3643, 2859 and 3653.

In one embodiment, the ISVD comprises CDRs (Chothia definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 2857-3662, preferably selected from SEQ ID NOs: 3395, 3376, 3447, 3446, 3643, 2859 and 3653.

In one embodiment, the ISVD comprises CDRs (IMGT definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 2857-3662, preferably selected from SEQ ID NOs: 3395, 3376, 3447, 3446, 3643, 2859 and 3653.

In another embodiment, the ISVD specifically binding to human PD-1 may have a sequence identity of more than 90%, such as more than 95% or even more than 99%, with SEQ ID NO: 3395, wherein the CDRs are as defined above. In one embodiment, the ISVD specifically binding to PD-1 comprises or consists of the amino acid sequence of SEQ ID NO: 3395.

In another embodiment, the ISVD specifically binding to human PD-1 may have a sequence identity of more than 90%, such as more than 95% or even more than 99%, with SEQ ID NO: 2857, wherein the CDRs are as defined above. In one embodiment, the ISVD specifically binding to PD-1 comprises or consists of the amino acid sequence of SEQ ID NO: 2857.

In another embodiment, the ISVD specifically binding to human PD-1 may have a sequence identity of more than 90%, such as more than 95% or even more than 99%, with SEQ ID NO: 2858, wherein the CDRs are as defined above. In one embodiment, the ISVD specifically binding to PD-1 comprises or consists of the amino acid sequence of SEQ ID NO: 2858.

In another embodiment, the ISVD specifically binding to human PD-1 may have a sequence identity of more than 90%, such as more than 95% or even more than 99%, with SEQ ID NO: 2859, wherein the CDRs are as defined above. In one embodiment, the ISVD specifically binding to PD-1 comprises or consists of the amino acid sequence of SEQ ID NO: 2859.

In another embodiment, the ISVD specifically binding to human PD-1 may have a sequence identity of more than 90%, such as more than 95% or even more than 99%, with SEQ ID NO: 2860, wherein the CDRs are as defined above. In one embodiment, the ISVD specifically binding to PD-1 comprises or consists of the amino acid sequence of SEQ ID NO: 2860.

In another embodiment, the ISVD specifically binding to human PD-1 may have a sequence identity of more than 90%, such as more than 95% or even more than 99%, with SEQ ID NO: 2861, wherein the CDRs are as defined above. In one embodiment, the ISVD specifically binding to PD-1 comprises or consists of the amino acid sequence of SEQ ID NO: 2861.

In another embodiment, the ISVD specifically binding to human PD-1 may have a sequence identity of more than 90%, such as more than 95% or even more than 99%, with SEQ ID NO: 2862, wherein the CDRs are as defined above. In one embodiment, the ISVD specifically binding to PD-1 comprises or consists of the amino acid sequence of SEQ ID NO: 2862.

In another embodiment, the ISVD specifically binding to human PD-1 may have a sequence identity of more than 90%, such as more than 95% or even more than 99%, with SEQ ID NO: 2863, wherein the CDRs are as defined above. In one embodiment, the ISVD specifically binding to PD-1 comprises or consists of the amino acid sequence of SEQ ID NO: 2863.

In another embodiment, the ISVD specifically binding to human PD-1 may have a sequence identity of more than 90%, such as more than 95% or even more than 99%, with SEQ ID NO: 2864, wherein the CDRs are as defined above. In one embodiment, the ISVD specifically binding to PD-1 comprises or consists of the amino acid sequence of SEQ ID NO: 2864.

In another embodiment, the ISVD specifically binding to human PD-1 may have a sequence identity of more than 90%, such as more than 95% or even more than 99%, with SEQ ID NO: 2865, wherein the CDRs are as defined above. In one embodiment, the ISVD specifically binding to PD-1 comprises or consists of the amino acid sequence of SEQ ID NO: 2865.

In another embodiment, the ISVD specifically binding to human PD-1 may have a sequence identity of more than 90%, such as more than 95% or even more than 99%, with SEQ ID NO: 2866, wherein the CDRs are as defined above. In one embodiment, the ISVD specifically binding to PD-1 comprises or consists of the amino acid sequence of SEQ ID NO: 2866.

In another embodiment, the ISVD specifically binding to human PD-1 may have a sequence identity of more than 90%, such as more than 95% or even more than 99%, with SEQ ID NO: 2867, wherein the CDRs are as defined above. In one embodiment, the ISVD specifically binding to PD-1 comprises or consists of the amino acid sequence of SEQ ID NO: 2867.

In another embodiment, the ISVD specifically binding to human PD-1 may have a sequence identity of more than 90%, such as more than 95% or even more than 99%, with SEQ ID NO: 2868, wherein the CDRs are as defined above. In one embodiment, the ISVD specifically binding to PD-1 comprises or consists of the amino acid sequence of SEQ ID NO: 2868.

In another embodiment, the ISVD specifically binding to human PD-1 may have a sequence identity of more than 90%, such as more than 95% or even more than 99%, with SEQ ID NO: 2869, wherein the CDRs are as defined above. In one embodiment, the ISVD specifically binding to PD-1 comprises or consists of the amino acid sequence of SEQ ID NO: 2869.

In another embodiment, the ISVD specifically binding to human PD-1 may have a sequence identity of more than 90%, such as more than 95% or even more than 99%, with SEQ ID NO: 2870, wherein the CDRs are as defined above. In one embodiment, the ISVD specifically binding to PD-1 comprises or consists of the amino acid sequence of SEQ ID NO: 2870.

In another embodiment, the ISVD specifically binding to human PD-1 may have a sequence identity of more than 90%, such as more than 95% or even more than 99%, with SEQ ID NO: 2871, wherein the CDRs are as defined above. In one embodiment, the ISVD specifically binding to PD-1 comprises or consists of the amino acid sequence of SEQ ID NO: 2871.

In another embodiment, the ISVD specifically binding to human PD-1 may have a sequence identity of more than 90%, such as more than 95% or even more than 99%, with SEQ ID NO: 2872, wherein the CDRs are as defined above. In one embodiment, the ISVD specifically binding to PD-1 comprises or consists of the amino acid sequence of SEQ ID NO: 2872.

In another embodiment, the ISVD specifically binding to human PD-1 may have a sequence identity of more than 90%, such as more than 95% or even more than 99%, with SEQ ID NO: 2873, wherein the CDRs are as defined above. In one embodiment, the ISVD specifically binding to PD-1 comprises or consists of the amino acid sequence of SEQ ID NO: 2873.

In another embodiment, the ISVD specifically binding to human PD-1 may have a sequence identity of more than 90%, such as more than 95% or even more than 99%, with SEQ ID NO: 2874, wherein the CDRs are as defined above. In one embodiment, the ISVD specifically binding to PD-1 comprises or consists of the amino acid sequence of SEQ ID NO: 2874.

In another embodiment, the ISVD specifically binding to human PD-1 may have a sequence identity of more than 90%, such as more than 95% or even more than 99%, with SEQ ID NO: 2875, wherein the CDRs are as defined above. In one embodiment, the ISVD specifically binding to PD-1 comprises or consists of the amino acid sequence of SEQ ID NO: 2875.

In another embodiment, the ISVD specifically binding to human PD-1 may have a sequence identity of more than 90%, such as more than 95% or even more than 99%, with SEQ ID NO: 2876, wherein the CDRs are as defined above. In one embodiment, the ISVD specifically binding to PD-1 comprises or consists of the amino acid sequence of SEQ ID NO: 2876.

In another embodiment, the ISVD specifically binding to human PD-1 may have a sequence identity of more than 90%, such as more than 95% or even more than 99%, with SEQ ID NO: 2877, wherein the CDRs are as defined above. In one embodiment, the ISVD specifically binding to PD-1 comprises or consists of the amino acid sequence of SEQ ID NO: 2877.

In another embodiment, the ISVD specifically binding to human PD-1 may have a sequence identity of more than 90%, such as more than 95% or even more than 99%, with SEQ ID NO: 2878, wherein the CDRs are as defined above. In one embodiment, the ISVD specifically binding to PD-1 comprises or consists of the amino acid sequence of SEQ ID NO: 2878.

In another embodiment, the ISVD specifically binding to human PD-1 may have a sequence identity of more than 90%, such as more than 95% or even more than 99%, with SEQ ID NO: 2879, wherein the CDRs are as defined above. In one embodiment, the ISVD specifically binding to PD-1 comprises or consists of the amino acid sequence of SEQ ID NO: 2879.

In another embodiment, the ISVD specifically binding to human PD-1 may have a sequence identity of more than 90%, such as more than 95% or even more than 99%, with SEQ ID NO: 2880, wherein the CDRs are as defined above. In one embodiment, the ISVD specifically binding to PD-1 comprises or consists of the amino acid sequence of SEQ ID NO: 2880.

In another embodiment, the ISVD specifically binding to human PD-1 may have a sequence identity of more than 90%, such as more than 95% or even more than 99%, with SEQ ID NO: 2881, wherein the CDRs are as defined above. In one embodiment, the ISVD specifically binding to PD-1 comprises or consists of the amino acid sequence of SEQ ID NO: 2881.

In another embodiment, the ISVD specifically binding to human PD-1 may have a sequence identity of more than 90%, such as more than 95% or even more than 99%, with SEQ ID NO: 2882, wherein the CDRs are as defined above. In one embodiment, the ISVD specifically binding to PD-1 comprises or consists of the amino acid sequence of SEQ ID NO: 2882.

In another embodiment, the ISVD specifically binding to human PD-1 may have a sequence identity of more than 90%, such as more than 95% or even more than 99%, with SEQ ID NO: 2883, wherein the CDRs are as defined above. In one embodiment, the ISVD specifically binding to PD-1 comprises or consists of the amino acid sequence of SEQ ID NO: 2883.

In another embodiment, the ISVD specifically binding to human PD-1 may have a sequence identity of more than 90%, such as more than 95% or even more than 99%, with SEQ ID NO: 2884, wherein the CDRs are as defined above. In one embodiment, the ISVD specifically binding to PD-1 comprises or consists of the amino acid sequence of SEQ ID NO: 2884.

In another embodiment, the ISVD specifically binding to human PD-1 may have a sequence identity of more than 90%, such as more than 95% or even more than 99%, with SEQ ID NO: 2885, wherein the CDRs are as defined above. In one embodiment, the ISVD specifically binding to PD-1 comprises or consists of the amino acid sequence of SEQ ID NO: 2885.

In another embodiment, the ISVD specifically binding to human PD-1 may have a sequence identity of more than 90%, such as more than 95% or even more than 99%, with SEQ ID NO: 2886, wherein the CDRs are as defined above. In one embodiment, the ISVD specifically binding to PD-1 comprises or consists of the amino acid sequence of SEQ ID NO: 2886.

In another embodiment, the ISVD specifically binding to human PD-1 may have a sequence identity of more than 90%, such as more than 95% or even more than 99%, with SEQ ID NO: 2887, wherein the CDRs are as defined above. In one embodiment, the ISVD specifically binding to PD-1 comprises or consists of the amino acid sequence of SEQ ID NO: 2887.

In another embodiment, the ISVD specifically binding to human PD-1 may have a sequence identity of more than 90%, such as more than 95% or even more than 99%, with SEQ ID NO: 2888, wherein the CDRs are as defined above. In one embodiment, the ISVD specifically binding to PD-1 comprises or consists of the amino acid sequence of SEQ ID NO: 2888.

In another embodiment, the ISVD specifically binding to human PD-1 may have a sequence identity of more than 90%, such as more than 95% or even more than 99%, with SEQ ID NO: 2889, wherein the CDRs are as defined above. In one embodiment, the ISVD specifically binding to PD-1 comprises or consists of the amino acid sequence of SEQ ID NO: 2889.

In another embodiment, the ISVD specifically binding to human PD-1 may have a sequence identity of more than 90%, such as more than 95% or even more than 99%, with SEQ ID NO: 2890, wherein the CDRs are as defined above. In one embodiment, the ISVD specifically binding to PD-1 comprises or consists of the amino acid sequence of SEQ ID NO: 2890.

In another embodiment, the ISVD specifically binding to human PD-1 may have a sequence identity of more than 90%, such as more than 95% or even more than 99%, with SEQ ID NO: 2891, wherein the CDRs are as defined above. In one embodiment, the ISVD specifically binding to PD-1 comprises or consists of the amino acid sequence of SEQ ID NO: 2891.

In another embodiment, the ISVD specifically binding to human PD-1 may have a sequence identity of more than 90%, such as more than 95% or even more than 99%, with SEQ ID NO: 2892, wherein the CDRs are as defined above. In one embodiment, the ISVD specifically binding to PD-1 comprises or consists of the amino acid sequence of SEQ ID NO: 2892.

In another embodiment, the ISVD specifically binding to human PD-1 may have a sequence identity of more than 90%, such as more than 95% or even more than 99%, with SEQ ID NO: 2893, wherein the CDRs are as defined above. In one embodiment, the ISVD specifically binding to PD-1 comprises or consists of the amino acid sequence of SEQ ID NO: 2893.

In another embodiment, the ISVD specifically binding to human PD-1 may have a sequence identity of more than 90%, such as more than 95% or even more than 99%, with SEQ ID NO: 2894, wherein the CDRs are as defined above. In one embodiment, the ISVD specifically binding to PD-1 comprises or consists of the amino acid sequence of SEQ ID NO: 2894.

In another embodiment, the ISVD specifically binding to human PD-1 may have a sequence identity of more than 90%, such as more than 95% or even more than 99%, with SEQ ID NO: 2895, wherein the CDRs are as defined above. In one embodiment, the ISVD specifically binding to PD-1 comprises or consists of the amino acid sequence of SEQ ID NO: 2895.

In another embodiment, the ISVD specifically binding to human PD-1 may have a sequence identity of more than 90%, such as more than 95% or even more than 99%, with SEQ ID NO: 2896, wherein the CDRs are as defined above. In one embodiment, the ISVD specifically binding to PD-1 comprises or consists of the amino acid sequence of SEQ ID NO: 2896.

In another embodiment, the ISVD specifically binding to human PD-1 may have a sequence identity of more than 90%, such as more than 95% or even more than 99%, with SEQ ID NO: 2897, wherein the CDRs are as defined above. In one embodiment, the ISVD specifically binding to PD-1 comprises or consists of the amino acid sequence of SEQ ID NO: 2897.

In another embodiment, the ISVD specifically binding to human PD-1 may have a sequence identity of more than 90%, such as more than 95% or even more than 99%, with SEQ ID NO: 2898, wherein the CDRs are as defined above. In one embodiment, the ISVD specifically binding to PD-1 comprises or consists of the amino acid sequence of SEQ ID NO: 2898.

In another embodiment, the ISVD specifically binding to human PD-1 may have a sequence identity of more than 90%, such as more than 95% or even more than 99%, with SEQ ID NO: 2899, wherein the CDRs are as defined above. In one embodiment, the ISVD specifically binding to PD-1 comprises or consists of the amino acid sequence of SEQ ID NO: 2899.

In another embodiment, the ISVD specifically binding to human PD-1 may have a sequence identity of more than 90%, such as more than 95% or even more than 99%, with SEQ ID NO: 2900, wherein the CDRs are as defined above. In one embodiment, the ISVD specifically binding to PD-1 comprises or consists of the amino acid sequence of SEQ ID NO: 2900.

In another embodiment, the ISVD specifically binding to human PD-1 may have a sequence identity of more than 90%, such as more than 95% or even more than 99%, with SEQ ID NO: 2901, wherein the CDRs are as defined above. In one embodiment, the ISVD specifically binding to PD-1 comprises or consists of the amino acid sequence of SEQ ID NO: 2901.

In another embodiment, the ISVD specifically binding to human PD-1 may have a sequence identity of more than 90%, such as more than 95% or even more than 99%, with SEQ ID NO: 2902, wherein the CDRs are as defined above. In one embodiment, the ISVD specifically binding to PD-1 comprises or consists of the amino acid sequence of SEQ ID NO: 2902.

In another embodiment, the ISVD specifically binding to human PD-1 may have a sequence identity of more than 90%, such as more than 95% or even more than 99%, with SEQ ID NO: 2903, wherein the CDRs are as defined above. In one embodiment, the ISVD specifically binding to PD-1 comprises or consists of the amino acid sequence of SEQ ID NO: 2903.

In another embodiment, the ISVD specifically binding to human PD-1 may have a sequence identity of more than 90%, such as more than 95% or even more than 99%, with SEQ ID NO: 2904, wherein the CDRs are as defined above. In one embodiment, the ISVD specifically binding to PD-1 comprises or consists of the amino acid sequence of SEQ ID NO: 2904.

In another embodiment, the ISVD specifically binding to human PD-1 may have a sequence identity of more than 90%, such as more than 95% or even more than 99%, with SEQ ID NO: 2905, wherein the CDRs are as defined above. In one embodiment, the ISVD specifically binding to PD-1 comprises or consists of the amino acid sequence of SEQ ID NO: 2905.

In another embodiment, the ISVD specifically binding to human PD-1 may have a sequence identity of more than 90%, such as more than 95% or even more than 99%, with SEQ ID NO: 2906, wherein the CDRs are as defined above. In one embodiment, the ISVD specifically binding to PD-1 comprises or consists of the amino acid sequence of SEQ ID NO: 2906.

In another embodiment, the ISVD specifically binding to human PD-1 may have a sequence identity of more than 90%, such as more than 95% or even more than 99%, with SEQ ID NO: 2907, wherein the CDRs are as defined above. In one embodiment, the ISVD specifically binding to PD-1 comprises or consists of the amino acid sequence of SEQ ID NO: 2907.

In another embodiment, the ISVD specifically binding to human PD-1 may have a sequence identity of more than 90%, such as more than 95% or even more than 99%, with SEQ ID NO: 2908, wherein the CDRs are as defined above. In one embodiment, the ISVD specifically binding to PD-1 comprises or consists of the amino acid sequence of SEQ ID NO: 2908.

In another embodiment, the ISVD specifically binding to human PD-1 may have a sequence identity of more than 90%, such as more than 95% or even more than 99%, with SEQ ID NO: 2909, wherein the CDRs are as defined above. In one embodiment, the ISVD specifically binding to PD-1 comprises or consists of the amino acid sequence of SEQ ID NO: 2909.

In another embodiment, the ISVD specifically binding to human PD-1 may have a sequence identity of more than 90%, such as more than 95% or even more than 99%, with SEQ ID NO: 2910, wherein the CDRs are as defined above. In one embodiment, the ISVD specifically binding to PD-1 comprises or consists of the amino acid sequence of SEQ ID NO: 2910.

In another embodiment, the ISVD specifically binding to human PD-1 may have a sequence identity of more than 90%, such as more than 95% or even more than 99%, with SEQ ID NO: 2911, wherein the CDRs are as defined above. In one embodiment, the ISVD specifically binding to PD-1 comprises or consists of the amino acid sequence of SEQ ID NO: 2911.

In another embodiment, the ISVD specifically binding to human PD-1 may have a sequence identity of more than 90%, such as more than 95% or even more than 99%, with SEQ ID NO: 2912, wherein the CDRs are as defined above. In one embodiment, the ISVD specifically binding to PD-1 comprises or consists of the amino acid sequence of SEQ ID NO: 2912.

In another embodiment, the ISVD specifically binding to human PD-1 may have a sequence identity of more than 90%, such as more than 95% or even more than 99%, with SEQ ID NO: 2913, wherein the CDRs are as defined above. In one embodiment, the ISVD specifically binding to PD-1 comprises or consists of the amino acid sequence of SEQ ID NO: 2913.

In another embodiment, the ISVD specifically binding to human PD-1 may have a sequence identity of more than 90%, such as more than 95% or even more than 99%, with SEQ ID NO: 2914, wherein the CDRs are as defined above. In one embodiment, the ISVD specifically binding to PD-1 comprises or consists of the amino acid sequence of SEQ ID NO: 2914.

In another embodiment, the ISVD specifically binding to human PD-1 may have a sequence identity of more than 90%, such as more than 95% or even more than 99%, with SEQ ID NO: 2915, wherein the CDRs are as defined above. In one embodiment, the ISVD specifically binding to PD-1 comprises or consists of the amino acid sequence of SEQ ID NO: 2915.

In another embodiment, the ISVD specifically binding to human PD-1 may have a sequence identity of more than 90%, such as more than 95% or even more than 99%, with SEQ ID NO: 2916, wherein the CDRs are as defined above. In one embodiment, the ISVD specifically binding to PD-1 comprises or consists of the amino acid sequence of SEQ ID NO: 2916.

In another embodiment, the ISVD specifically binding to human PD-1 may have a sequence identity of more than 90%, such as more than 95% or even more than 99%, with SEQ ID NO: 2917, wherein the CDRs are as defined above. In one embodiment, the ISVD specifically binding to PD-1 comprises or consists of the amino acid sequence of SEQ ID NO: 2917.

In another embodiment, the ISVD specifically binding to human PD-1 may have a sequence identity of more than 90%, such as more than 95% or even more than 99%, with SEQ ID NO: 2918, wherein the CDRs are as defined above. In one embodiment, the ISVD specifically binding to PD-1 comprises or consists of the amino acid sequence of SEQ ID NO: 2918.

In another embodiment, the ISVD specifically binding to human PD-1 may have a sequence identity of more than 90%, such as more than 95% or even more than 99%, with SEQ ID NO: 2919, wherein the CDRs are as defined above. In one embodiment, the ISVD specifically binding to PD-1 comprises or consists of the amino acid sequence of SEQ ID NO: 2919.

In another embodiment, the ISVD specifically binding to human PD-1 may have a sequence identity of more than 90%, such as more than 95% or even more than 99%, with SEQ ID NO: 2920, wherein the CDRs are as defined above. In one embodiment, the ISVD specifically binding to PD-1 comprises or consists of the amino acid sequence of SEQ ID NO: 2920.

In another embodiment, the ISVD specifically binding to human PD-1 may have a sequence identity of more than 90%, such as more than 95% or even more than 99%, with SEQ ID NO: 2921, wherein the CDRs are as defined above. In one embodiment, the ISVD specifically binding to PD-1 comprises or consists of the amino acid sequence of SEQ ID NO: 2921.

In another embodiment, the ISVD specifically binding to human PD-1 may have a sequence identity of more than 90%, such as more than 95% or even more than 99%, with SEQ ID NO: 2922, wherein the CDRs are as defined above. In one embodiment, the ISVD specifically binding to PD-1 comprises or consists of the amino acid sequence of SEQ ID NO: 2922.

In another embodiment, the ISVD specifically binding to human PD-1 may have a sequence identity of more than 90%, such as more than 95% or even more than 99%, with SEQ ID NO: 2923, wherein the CDRs are as defined above. In one embodiment, the ISVD specifically binding to PD-1 comprises or consists of the amino acid sequence of SEQ ID NO: 2923.

In another embodiment, the ISVD specifically binding to human PD-1 may have a sequence identity of more than 90%, such as more than 95% or even more than 99%, with SEQ ID NO: 2924, wherein the CDRs are as defined above. In one embodiment, the ISVD specifically binding to PD-1 comprises or consists of the amino acid sequence of SEQ ID NO: 2924.

In another embodiment, the ISVD specifically binding to human PD-1 may have a sequence identity of more than 90%, such as more than 95% or even more than 99%, with SEQ ID NO: 2925, wherein the CDRs are as defined above. In one embodiment, the ISVD specifically binding to PD-1 comprises or consists of the amino acid sequence of SEQ ID NO: 2925.

In another embodiment, the ISVD specifically binding to human PD-1 may have a sequence identity of more than 90%, such as more than 95% or even more than 99%, with SEQ ID NO: 2926, wherein the CDRs are as defined above. In one embodiment, the ISVD specifically binding to PD-1 comprises or consists of the amino acid sequence of SEQ ID NO: 2926.

In another embodiment, the ISVD specifically binding to human PD-1 may have a sequence identity of more than 90%, such as more than 95% or even more than 99%, with SEQ ID NO: 2927, wherein the CDRs are as defined above. In one embodiment, the ISVD specifically binding to PD-1 comprises or consists of the amino acid sequence of SEQ ID NO: 2927.

In another embodiment, the ISVD specifically binding to human PD-1 may have a sequence identity of more than 90%, such as more than 95% or even more than 99%, with SEQ ID NO: 2928, wherein the CDRs are as defined above. In one embodiment, the ISVD specifically binding to PD-1 comprises or consists of the amino acid sequence of SEQ ID NO: 2928.

In another embodiment, the ISVD specifically binding to human PD-1 may have a sequence identity of more than 90%, such as more than 95% or even more than 99%, with SEQ ID NO: 2929, wherein the CDRs are as defined above. In one embodiment, the ISVD specifically binding to PD-1 comprises or consists of the amino acid sequence of SEQ ID NO: 2929.

In another embodiment, the ISVD specifically binding to human PD-1 may have a sequence identity of more than 90%, such as more than 95% or even more than 99%, with SEQ ID NO: 2930, wherein the CDRs are as defined above. In one embodiment, the ISVD specifically binding to PD-1 comprises or consists of the amino acid sequence of SEQ ID NO: 2930.

In another embodiment, the ISVD specifically binding to human PD-1 may have a sequence identity of more than 90%, such as more than 95% or even more than 99%, with SEQ ID NO: 2931, wherein the CDRs are as defined above. In one embodiment, the ISVD specifically binding to PD-1 comprises or consists of the amino acid sequence of SEQ ID NO: 2931.

In another embodiment, the ISVD specifically binding to human PD-1 may have a sequence identity of more than 90%, such as more than 95% or even more than 99%, with SEQ ID NO: 2932, wherein the CDRs are as defined above. In one embodiment, the ISVD specifically binding to PD-1 comprises or consists of the amino acid sequence of SEQ ID NO: 2932.

In another embodiment, the ISVD specifically binding to human PD-1 may have a sequence identity of more than 90%, such as more than 95% or even more than 99%, with SEQ ID NO: 2933, wherein the CDRs are as defined above. In one embodiment, the ISVD specifically binding to PD-1 comprises or consists of the amino acid sequence of SEQ ID NO: 2933.

In another embodiment, the ISVD specifically binding to human PD-1 may have a sequence identity of more than 90%, such as more than 95% or even more than 99%, with SEQ ID NO: 2934, wherein the CDRs are as defined above. In one embodiment, the ISVD specifically binding to PD-1 comprises or consists of the amino acid sequence of SEQ ID NO: 2934.

In another embodiment, the ISVD specifically binding to human PD-1 may have a sequence identity of more than 90%, such as more than 95% or even more than 99%, with SEQ ID NO: 2935, wherein the CDRs are as defined above. In one embodiment, the ISVD specifically binding to PD-1 comprises or consists of the amino acid sequence of SEQ ID NO: 2935.

In another embodiment, the ISVD specifically binding to human PD-1 may have a sequence identity of more than 90%, such as more than 95% or even more than 99%, with SEQ ID NO: 2936, wherein the CDRs are as defined above. In one embodiment, the ISVD specifically binding to PD-1 comprises or consists of the amino acid sequence of SEQ ID NO: 2936.

In another embodiment, the ISVD specifically binding to human PD-1 may have a sequence identity of more than 90%, such as more than 95% or even more than 99%, with SEQ ID NO: 2937, wherein the CDRs are as defined above. In one embodiment, the ISVD specifically binding to PD-1 comprises or consists of the amino acid sequence of SEQ ID NO: 2937.

In another embodiment, the ISVD specifically binding to human PD-1 may have a sequence identity of more than 90%, such as more than 95% or even more than 99%, with SEQ ID NO: 2938, wherein the CDRs are as defined above. In one embodiment, the ISVD specifically binding to PD-1 comprises or consists of the amino acid sequence of SEQ ID NO: 2938.

In another embodiment, the ISVD specifically binding to human PD-1 may have a sequence identity of more than 90%, such as more than 95% or even more than 99%, with SEQ ID NO: 2939, wherein the CDRs are as defined above. In one embodiment, the ISVD specifically binding to PD-1 comprises or consists of the amino acid sequence of SEQ ID NO: 2939.

In another embodiment, the ISVD specifically binding to human PD-1 may have a sequence identity of more than 90%, such as more than 95% or even more than 99%, with SEQ ID NO: 2940, wherein the CDRs are as defined above. In one embodiment, the ISVD specifically binding to PD-1 comprises or consists of the amino acid sequence of SEQ ID NO: 2940.

In another embodiment, the ISVD specifically binding to human PD-1 may have a sequence identity of more than 90%, such as more than 95% or even more than 99%, with SEQ ID NO: 2941, wherein the CDRs are as defined above. In one embodiment, the ISVD specifically binding to PD-1 comprises or consists of the amino acid sequence of SEQ ID NO: 2941.

In another embodiment, the ISVD specifically binding to human PD-1 may have a sequence identity of more than 90%, such as more than 95% or even more than 99%, with SEQ ID NO: 2942, wherein the CDRs are as defined above. In one embodiment, the ISVD specifically binding to PD-1 comprises or consists of the amino acid sequence of SEQ ID NO: 2942.

In another embodiment, the ISVD specifically binding to human PD-1 may have a sequence identity of more than 90%, such as more than 95% or even more than 99%, with SEQ ID NO: 2943, wherein the CDRs are as defined above. In one embodiment, the ISVD specifically binding to PD-1 comprises or consists of the amino acid sequence of SEQ ID NO: 2943.

In another embodiment, the ISVD specifically binding to human PD-1 may have a sequence identity of more than 90%, such as more than 95% or even more than 99%, with SEQ ID NO: 2944, wherein the CDRs are as defined above. In one embodiment, the ISVD specifically binding to PD-1 comprises or consists of the amino acid sequence of SEQ ID NO: 2944.

In another embodiment, the ISVD specifically binding to human PD-1 may have a sequence identity of more than 90%, such as more than 95% or even more than 99%, with SEQ ID NO: 2945, wherein the CDRs are as defined above. In one embodiment, the ISVD specifically binding to PD-1 comprises or consists of the amino acid sequence of SEQ ID NO: 2945.

In another embodiment, the ISVD specifically binding to human PD-1 may have a sequence identity of more than 90%, such as more than 95% or even more than 99%, with SEQ ID NO: 2946, wherein the CDRs are as defined above. In one embodiment, the ISVD specifically binding to PD-1 comprises or consists of the amino acid sequence of SEQ ID NO: 2946.

In another embodiment, the ISVD specifically binding to human PD-1 may have a sequence identity of more than 90%, such as more than 95% or even more than 99%, with SEQ ID NO: 2947, wherein the CDRs are as defined above. In one embodiment, the ISVD specifically binding to PD-1 comprises or consists of the amino acid sequence of SEQ ID NO: 2947.

In another embodiment, the ISVD specifically binding to human PD-1 may have a sequence identity of more than 90%, such as more than 95% or even more than 99%, with SEQ ID NO: 2948, wherein the CDRs are as defined above. In one embodiment, the ISVD specifically binding to PD-1 comprises or consists of the amino acid sequence of SEQ ID NO: 2948.

In another embodiment, the ISVD specifically binding to human PD-1 may have a sequence identity of more than 90%, such as more than 95% or even more than 99%, with SEQ ID NO: 2949, wherein the CDRs are as defined above. In one embodiment, the ISVD specifically binding to PD-1 comprises or consists of the amino acid sequence of SEQ ID NO: 2949.

In another embodiment, the ISVD specifically binding to human PD-1 may have a sequence identity of more than 90%, such as more than 95% or even more than 99%, with SEQ ID NO: 2950, wherein the CDRs are as defined above. In one embodiment, the ISVD specifically binding to PD-1 comprises or consists of the amino acid sequence of SEQ ID NO: 2950.

In another embodiment, the ISVD specifically binding to human PD-1 may have a sequence identity of more than 90%, such as more than 95% or even more than 99%, with SEQ ID NO: 2951, wherein the CDRs are as defined above. In one embodiment, the ISVD specifically binding to PD-1 comprises or consists of the amino acid sequence of SEQ ID NO: 2951.

In another embodiment, the ISVD specifically binding to human PD-1 may have a sequence identity of more than 90%, such as more than 95% or even more than 99%, with SEQ ID NO: 2952, wherein the CDRs are as defined above. In one embodiment, the ISVD specifically binding to PD-1 comprises or consists of the amino acid sequence of SEQ ID NO: 2952.

In another embodiment, the ISVD specifically binding to human PD-1 may have a sequence identity of more than 90%, such as more than 95% or even more than 99%, with SEQ ID NO: 2953, wherein the CDRs are as defined above. In one embodiment, the ISVD specifically binding to PD-1 comprises or consists of the amino acid sequence of SEQ ID NO: 2953.

In another embodiment, the ISVD specifically binding to human PD-1 may have a sequence identity of more than 90%, such as more than 95% or even more than 99%, with SEQ ID NO: 2954, wherein the CDRs are as defined above. In one embodiment, the ISVD specifically binding to PD-1 comprises or consists of the amino acid sequence of SEQ ID NO: 2954.

In another embodiment, the ISVD specifically binding to human PD-1 may have a sequence identity of more than 90%, such as more than 95% or even more than 99%, with SEQ ID NO: 2955, wherein the CDRs are as defined above. In one embodiment, the ISVD specifically binding to PD-1 comprises or consists of the amino acid sequence of SEQ ID NO: 2955.

In another embodiment, the ISVD specifically binding to human PD-1 may have a sequence identity of more than 90%, such as more than 95% or even more than 99%, with SEQ ID NO: 2956, wherein the CDRs are as defined above. In one embodiment, the ISVD specifically binding to PD-1 comprises or consists of the amino acid sequence of SEQ ID NO: 2956.

In another embodiment, the ISVD specifically binding to human PD-1 may have a sequence identity of more than 90%, such as more than 95% or even more than 99%, with SEQ ID NO: 2957, wherein the CDRs are as defined above. In one embodiment, the ISVD specifically binding to PD-1 comprises or consists of the amino acid sequence of SEQ ID NO: 2957.

In another embodiment, the ISVD specifically binding to human PD-1 may have a sequence identity of more than 90%, such as more than 95% or even more than 99%, with SEQ ID NO: 2958, wherein the CDRs are as defined above. In one embodiment, the ISVD specifically binding to PD-1 comprises or consists of the amino acid sequence of SEQ ID NO: 2958.

In another embodiment, the ISVD specifically binding to human PD-1 may have a sequence identity of more than 90%, such as more than 95% or even more than 99%, with SEQ ID NO: 2959, wherein the CDRs are as defined above. In one embodiment, the ISVD specifically binding to PD-1 comprises or consists of the amino acid sequence of SEQ ID NO: 2959.

In another embodiment, the ISVD specifically binding to human PD-1 may have a sequence identity of more than 90%, such as more than 95% or even more than 99%, with SEQ ID NO: 2960, wherein the CDRs are as defined above. In one embodiment, the ISVD specifically binding to PD-1 comprises or consists of the amino acid sequence of SEQ ID NO: 2960.

In another embodiment, the ISVD specifically binding to human PD-1 may have a sequence identity of more than 90%, such as more than 95% or even more than 99%, with SEQ ID NO: 2961, wherein the CDRs are as defined above. In one embodiment, the ISVD specifically binding to PD-1 comprises or consists of the amino acid sequence of SEQ ID NO: 2961.

In another embodiment, the ISVD specifically binding to human PD-1 may have a sequence identity of more than 90%, such as more than 95% or even more than 99%, with SEQ ID NO: 2962, wherein the CDRs are as defined above. In one embodiment, the ISVD specifically binding to PD-1 comprises or consists of the amino acid sequence of SEQ ID NO: 2962.

In another embodiment, the ISVD specifically binding to human PD-1 may have a sequence identity of more than 90%, such as more than 95% or even more than 99%, with SEQ ID NO: 2963, wherein the CDRs are as defined above. In one embodiment, the ISVD specifically binding to PD-1 comprises or consists of the amino acid sequence of SEQ ID NO: 2963.

In another embodiment, the ISVD specifically binding to human PD-1 may have a sequence identity of more than 90%, such as more than 95% or even more than 99%, with SEQ ID NO: 2964, wherein the CDRs are as defined above. In one embodiment, the ISVD specifically binding to PD-1 comprises or consists of the amino acid sequence of SEQ ID NO: 2964.

In another embodiment, the ISVD specifically binding to human PD-1 may have a sequence identity of more than 90%, such as more than 95% or even more than 99%, with SEQ ID NO: 2965, wherein the CDRs are as defined above. In one embodiment, the ISVD specifically binding to PD-1 comprises or consists of the amino acid sequence of SEQ ID NO: 2965.

In another embodiment, the ISVD specifically binding to human PD-1 may have a sequence identity of more than 90%, such as more than 95% or even more than 99%, with SEQ ID NO: 2966, wherein the CDRs are as defined above. In one embodiment, the ISVD specifically binding to PD-1 comprises or consists of the amino acid sequence of SEQ ID NO: 2966.

In another embodiment, the ISVD specifically binding to human PD-1 may have a sequence identity of more than 90%, such as more than 95% or even more than 99%, with SEQ ID NO: 2967, wherein the CDRs are as defined above. In one embodiment, the ISVD specifically binding to PD-1 comprises or consists of the amino acid sequence of SEQ ID NO: 2967.

In another embodiment, the ISVD specifically binding to human PD-1 may have a sequence identity of more than 90%, such as more than 95% or even more than 99%, with SEQ ID NO: 2968, wherein the CDRs are as defined above. In one embodiment, the ISVD specifically binding to PD-1 comprises or consists of the amino acid sequence of SEQ ID NO: 2968.

In another embodiment, the ISVD specifically binding to human PD-1 may have a sequence identity of more than 90%, such as more than 95% or even more than 99%, with SEQ ID NO: 2969, wherein the CDRs are as defined above. In one embodiment, the ISVD specifically binding to PD-1 comprises or consists of the amino acid sequence of SEQ ID NO: 2969.

In another embodiment, the ISVD specifically binding to human PD-1 may have a sequence identity of more than 90%, such as more than 95% or even more than 99%, with SEQ ID NO: 2970, wherein the CDRs are as defined above. In one embodiment, the ISVD specifically binding to PD-1 comprises or consists of the amino acid sequence of SEQ ID NO: 2970.

In another embodiment, the ISVD specifically binding to human PD-1 may have a sequence identity of more than 90%, such as more than 95% or even more than 99%, with SEQ ID NO: 2971, wherein the CDRs are as defined above. In one embodiment, the ISVD specifically binding to PD-1 comprises or consists of the amino acid sequence of SEQ ID NO: 2971.

In another embodiment, the ISVD specifically binding to human PD-1 may have a sequence identity of more than 90%, such as more than 95% or even more than 99%, with SEQ ID NO: 2972, wherein the CDRs are as defined above. In one embodiment, the ISVD specifically binding to PD-1 comprises or consists of the amino acid sequence of SEQ ID NO: 2972.

In another embodiment, the ISVD specifically binding to human PD-1 may have a sequence identity of more than 90%, such as more than 95% or even more than 99%, with SEQ ID NO: 2973, wherein the CDRs are as defined above. In one embodiment, the ISVD specifically binding to PD-1 comprises or consists of the amino acid sequence of SEQ ID NO: 2973.

In another embodiment, the ISVD specifically binding to human PD-1 may have a sequence identity of more than 90%, such as more than 95% or even more than 99%, with SEQ ID NO: 2974, wherein the CDRs are as defined above. In one embodiment, the ISVD specifically binding to PD-1 comprises or consists of the amino acid sequence of SEQ ID NO: 2974.

In another embodiment, the ISVD specifically binding to human PD-1 may have a sequence identity of more than 90%, such as more than 95% or even more than 99%, with SEQ ID NO: 2975, wherein the CDRs are as defined above. In one embodiment, the ISVD specifically binding to PD-1 comprises or consists of the amino acid sequence of SEQ ID NO: 2975.

In another embodiment, the ISVD specifically binding to human PD-1 may have a sequence identity of more than 90%, such as more than 95% or even more than 99%, with SEQ ID NO: 2976, wherein the CDRs are as defined above. In one embodiment, the ISVD specifically binding to PD-1 comprises or consists of the amino acid sequence of SEQ ID NO: 2976.

In another embodiment, the ISVD specifically binding to human PD-1 may have a sequence identity of more than 90%, such as more than 95% or even more than 99%, with SEQ ID NO: 2977, wherein the CDRs are as defined above. In one embodiment, the ISVD specifically binding to PD-1 comprises or consists of the amino acid sequence of SEQ ID NO: 2977.

In another embodiment, the ISVD specifically binding to human PD-1 may have a sequence identity of more than 90%, such as more than 95% or even more than 99%, with SEQ ID NO: 2978, wherein the CDRs are as defined above. In one embodiment, the ISVD specifically binding to PD-1 comprises or consists of the amino acid sequence of SEQ ID NO: 2978.

In another embodiment, the ISVD specifically binding to human PD-1 may have a sequence identity of more than 90%, such as more than 95% or even more than 99%, with SEQ ID NO: 2979, wherein the CDRs are as defined above. In one embodiment, the ISVD specifically binding to PD-1 comprises or consists of the amino acid sequence of SEQ ID NO: 2979.

In another embodiment, the ISVD specifically binding to human PD-1 may have a sequence identity of more than 90%, such as more than 95% or even more than 99%, with SEQ ID NO: 2980, wherein the CDRs are as defined above. In one embodiment, the ISVD specifically binding to PD-1 comprises or consists of the amino acid sequence of SEQ ID NO: 2980.

In another embodiment, the ISVD specifically binding to human PD-1 may have a sequence identity of more than 90%, such as more than 95% or even more than 99%, with SEQ ID NO: 2981, wherein the CDRs are as defined above. In one embodiment, the ISVD specifically binding to PD-1 comprises or consists of the amino acid sequence of SEQ ID NO: 2981.

In another embodiment, the ISVD specifically binding to human PD-1 may have a sequence identity of more than 90%, such as more than 95% or even more than 99%, with SEQ ID NO: 2982, wherein the CDRs are as defined above. In one embodiment, the ISVD specifically binding to PD-1 comprises or consists of the amino acid sequence of SEQ ID NO: 2982.

In another embodiment, the ISVD specifically binding to human PD-1 may have a sequence identity of more than 90%, such as more than 95% or even more than 99%, with SEQ ID NO: 2983, wherein the CDRs are as defined above. In one embodiment, the ISVD specifically binding to PD-1 comprises or consists of the amino acid sequence of SEQ ID NO: 2983.

In another embodiment, the ISVD specifically binding to human PD-1 may have a sequence identity of more than 90%, such as more than 95% or even more than 99%, with SEQ ID NO: 2984, wherein the CDRs are as defined above. In one embodiment, the ISVD specifically binding to PD-1 comprises or consists of the amino acid sequence of SEQ ID NO: 2984.

In another embodiment, the ISVD specifically binding to human PD-1 may have a sequence identity of more than 90%, such as more than 95% or even more than 99%, with SEQ ID NO: 2985, wherein the CDRs are as defined above. In one embodiment, the ISVD specifically binding to PD-1 comprises or consists of the amino acid sequence of SEQ ID NO: 2985.

In another embodiment, the ISVD specifically binding to human PD-1 may have a sequence identity of more than 90%, such as more than 95% or even more than 99%, with SEQ ID NO: 2986, wherein the CDRs are as defined above. In one embodiment, the ISVD specifically binding to PD-1 comprises or consists of the amino acid sequence of SEQ ID NO: 2986.

In another embodiment, the ISVD specifically binding to human PD-1 may have a sequence identity of more than 90%, such as more than 95% or even more than 99%, with SEQ ID NO: 2987, wherein the CDRs are as defined above. In one embodiment, the ISVD specifically binding to PD-1 comprises or consists of the amino acid sequence of SEQ ID NO: 2987.

In another embodiment, the ISVD specifically binding to human PD-1 may have a sequence identity of more than 90%, such as more than 95% or even more than 99%, with SEQ ID NO: 2988, wherein the CDRs are as defined above. In one embodiment, the ISVD specifically binding to PD-1 comprises or consists of the amino acid sequence of SEQ ID NO: 2988.

In another embodiment, the ISVD specifically binding to human PD-1 may have a sequence identity of more than 90%, such as more than 95% or even more than 99%, with SEQ ID NO: 2989, wherein the CDRs are as defined above. In one embodiment, the ISVD specifically binding to PD-1 comprises or consists of the amino acid sequence of SEQ ID NO: 2989.

In another embodiment, the ISVD specifically binding to human PD-1 may have a sequence identity of more than 90%, such as more than 95% or even more than 99%, with SEQ ID NO: 2990, wherein the CDRs are as defined above. In one embodiment, the ISVD specifically binding to PD-1 comprises or consists of the amino acid sequence of SEQ ID NO: 2990.

In another embodiment, the ISVD specifically binding to human PD-1 may have a sequence identity of more than 90%, such as more than 95% or even more than 99%, with SEQ ID NO: 2991, wherein the CDRs are as defined above. In one embodiment, the ISVD specifically binding to PD-1 comprises or consists of the amino acid sequence of SEQ ID NO: 2991.

In another embodiment, the ISVD specifically binding to human PD-1 may have a sequence identity of more than 90%, such as more than 95% or even more than 99%, with SEQ ID NO: 2992, wherein the CDRs are as defined above. In one embodiment, the ISVD specifically binding to PD-1 comprises or consists of the amino acid sequence of SEQ ID NO: 2992.

In another embodiment, the ISVD specifically binding to human PD-1 may have a sequence identity of more than 90%, such as more than 95% or even more than 99%, with SEQ ID NO: 2993, wherein the CDRs are as defined above. In one embodiment, the ISVD specifically binding to PD-1 comprises or consists of the amino acid sequence of SEQ ID NO: 2993.

In another embodiment, the ISVD specifically binding to human PD-1 may have a sequence identity of more than 90%, such as more than 95% or even more than 99%, with SEQ ID NO: 2994, wherein the CDRs are as defined above. In one embodiment, the ISVD specifically binding to PD-1 comprises or consists of the amino acid sequence of SEQ ID NO: 2994.

In another embodiment, the ISVD specifically binding to human PD-1 may have a sequence identity of more than 90%, such as more than 95% or even more than 99%, with SEQ ID NO: 2995, wherein the CDRs are as defined above. In one embodiment, the ISVD specifically binding to PD-1 comprises or consists of the amino acid sequence of SEQ ID NO: 2995.

In another embodiment, the ISVD specifically binding to human PD-1 may have a sequence identity of more than 90%, such as more than 95% or even more than 99%, with SEQ ID NO: 2996, wherein the CDRs are as defined above. In one embodiment, the ISVD specifically binding to PD-1 comprises or consists of the amino acid sequence of SEQ ID NO: 2996.

In another embodiment, the ISVD specifically binding to human PD-1 may have a sequence identity of more than 90%, such as more than 95% or even more than 99%, with SEQ ID NO: 2997, wherein the CDRs are as defined above. In one embodiment, the ISVD specifically binding to PD-1 comprises or consists of the amino acid sequence of SEQ ID NO: 2997.

In another embodiment, the ISVD specifically binding to human PD-1 may have a sequence identity of more than 90%, such as more than 95% or even more than 99%, with SEQ ID NO: 2998, wherein the CDRs are as defined above. In one embodiment, the ISVD specifically binding to PD-1 comprises or consists of the amino acid sequence of SEQ ID NO: 2998.

In another embodiment, the ISVD specifically binding to human PD-1 may have a sequence identity of more than 90%, such as more than 95% or even more than 99%, with SEQ ID NO: 2999, wherein the CDRs are as defined above. In one embodiment, the ISVD specifically binding to PD-1 comprises or consists of the amino acid sequence of SEQ ID NO: 2999.

In another embodiment, the ISVD specifically binding to human PD-1 may have a sequence identity of more than 90%, such as more than 95% or even more than 99%, with SEQ ID NO: 3000, wherein the CDRs are as defined above. In one embodiment, the ISVD specifically binding to PD-1 comprises or consists of the amino acid sequence of SEQ ID NO: 3000.

In another embodiment, the ISVD specifically binding to human PD-1 may have a sequence identity of more than 90%, such as more than 95% or even more than 99%, with SEQ ID NO: 3001, wherein the CDRs are as defined above. In one embodiment, the ISVD specifically binding to PD-1 comprises or consists of the amino acid sequence of SEQ ID NO: 3001.

In another embodiment, the ISVD specifically binding to human PD-1 may have a sequence identity of more than 90%, such as more than 95% or even more than 99%, with SEQ ID NO: 3002, wherein the CDRs are as defined above. In one embodiment, the ISVD specifically binding to PD-1 comprises or consists of the amino acid sequence of SEQ ID NO: 3002.

In another embodiment, the ISVD specifically binding to human PD-1 may have a sequence identity of more than 90%, such as more than 95% or even more than 99%, with SEQ ID NO: 3003, wherein the CDRs are as defined above. In one embodiment, the ISVD specifically binding to PD-1 comprises or consists of the amino acid sequence of SEQ ID NO: 3003.

In another embodiment, the ISVD specifically binding to human PD-1 may have a sequence identity of more than 90%, such as more than 95% or even more than 99%, with SEQ ID NO: 3004, wherein the CDRs are as defined above. In one embodiment, the ISVD specifically binding to PD-1 comprises or consists of the amino acid sequence of SEQ ID NO: 3004.

In another embodiment, the ISVD specifically binding to human PD-1 may have a sequence identity of more than 90%, such as more than 95% or even more than 99%, with SEQ ID NO: 3005, wherein the CDRs are as defined above. In one embodiment, the ISVD specifically binding to PD-1 comprises or consists of the amino acid sequence of SEQ ID NO: 3005.

In another embodiment, the ISVD specifically binding to human PD-1 may have a sequence identity of more than 90%, such as more than 95% or even more than 99%, with SEQ ID NO: 3006, wherein the CDRs are as defined above. In one embodiment, the ISVD specifically binding to PD-1 comprises or consists of the amino acid sequence of SEQ ID NO: 3006.

In another embodiment, the ISVD specifically binding to human PD-1 may have a sequence identity of more than 90%, such as more than 95% or even more than 99%, with SEQ ID NO: 3007, wherein the CDRs are as defined above. In one embodiment, the ISVD specifically binding to PD-1 comprises or consists of the amino acid sequence of SEQ ID NO: 3007.

In another embodiment, the ISVD specifically binding to human PD-1 may have a sequence identity of more than 90%, such as more than 95% or even more than 99%, with SEQ ID NO: 3008, wherein the CDRs are as defined above. In one embodiment, the ISVD specifically binding to PD-1 comprises or consists of the amino acid sequence of SEQ ID NO: 3008.

In another embodiment, the ISVD specifically binding to human PD-1 may have a sequence identity of more than 90%, such as more than 95% or even more than 99%, with SEQ ID NO: 3009, wherein the CDRs are as defined above. In one embodiment, the ISVD specifically binding to PD-1 comprises or consists of the amino acid sequence of SEQ ID NO: 3009.

In another embodiment, the ISVD specifically binding to human PD-1 may have a sequence identity of more than 90%, such as more than 95% or even more than 99%, with SEQ ID NO: 3010, wherein the CDRs are as defined above. In one embodiment, the ISVD specifically binding to PD-1 comprises or consists of the amino acid sequence of SEQ ID NO: 3010.

In another embodiment, the ISVD specifically binding to human PD-1 may have a sequence identity of more than 90%, such as more than 95% or even more than 99%, with SEQ ID NO: 3011, wherein the CDRs are as defined above. In one embodiment, the ISVD specifically binding to PD-1 comprises or consists of the amino acid sequence of SEQ ID NO: 3011.

In another embodiment, the ISVD specifically binding to human PD-1 may have a sequence identity of more than 90%, such as more than 95% or even more than 99%, with SEQ ID NO: 3012, wherein the CDRs are as defined above. In one embodiment, the ISVD specifically binding to PD-1 comprises or consists of the amino acid sequence of SEQ ID NO: 3012.

In another embodiment, the ISVD specifically binding to human PD-1 may have a sequence identity of more than 90%, such as more than 95% or even more than 99%, with SEQ ID NO: 3013, wherein the CDRs are as defined above. In one embodiment, the ISVD specifically binding to PD-1 comprises or consists of the amino acid sequence of SEQ ID NO: 3013.

In another embodiment, the ISVD specifically binding to human PD-1 may have a sequence identity of more than 90%, such as more than 95% or even more than 99%, with SEQ ID NO: 3014, wherein the CDRs are as defined above. In one embodiment, the ISVD specifically binding to PD-1 comprises or consists of the amino acid sequence of SEQ ID NO: 3014.

In another embodiment, the ISVD specifically binding to human PD-1 may have a sequence identity of more than 90%, such as more than 95% or even more than 99%, with SEQ ID NO: 3015, wherein the CDRs are as defined above. In one embodiment, the ISVD specifically binding to PD-1 comprises or consists of the amino acid sequence of SEQ ID NO: 3015.

In another embodiment, the ISVD specifically binding to human PD-1 may have a sequence identity of more than 90%, such as more than 95% or even more than 99%, with SEQ ID NO: 3016, wherein the CDRs are as defined above. In one embodiment, the ISVD specifically binding to PD-1 comprises or consists of the amino acid sequence of SEQ ID NO: 3016.

In another embodiment, the ISVD specifically binding to human PD-1 may have a sequence identity of more than 90%, such as more than 95% or even more than 99%, with SEQ ID NO: 3017, wherein the CDRs are as defined above. In one embodiment, the ISVD specifically binding to PD-1 comprises or consists of the amino acid sequence of SEQ ID NO: 3017.

In another embodiment, the ISVD specifically binding to human PD-1 may have a sequence identity of more than 90%, such as more than 95% or even more than 99%, with SEQ ID NO: 3018, wherein the CDRs are as defined above. In one embodiment, the ISVD specifically binding to PD-1 comprises or consists of the amino acid sequence of SEQ ID NO: 3018.

In another embodiment, the ISVD specifically binding to human PD-1 may have a sequence identity of more than 90%, such as more than 95% or even more than 99%, with SEQ ID NO: 3019, wherein the CDRs are as defined above. In one embodiment, the ISVD specifically binding to PD-1 comprises or consists of the amino acid sequence of SEQ ID NO: 3019.

In another embodiment, the ISVD specifically binding to human PD-1 may have a sequence identity of more than 90%, such as more than 95% or even more than 99%, with SEQ ID NO: 3020, wherein the CDRs are as defined above. In one embodiment, the ISVD specifically binding to PD-1 comprises or consists of the amino acid sequence of SEQ ID NO: 3020.

In another embodiment, the ISVD specifically binding to human PD-1 may have a sequence identity of more than 90%, such as more than 95% or even more than 99%, with SEQ ID NO: 3021, wherein the CDRs are as defined above. In one embodiment, the ISVD specifically binding to PD-1 comprises or consists of the amino acid sequence of SEQ ID NO: 3021.

In another embodiment, the ISVD specifically binding to human PD-1 may have a sequence identity of more than 90%, such as more than 95% or even more than 99%, with SEQ ID NO: 3022, wherein the CDRs are as defined above. In one embodiment, the ISVD specifically binding to PD-1 comprises or consists of the amino acid sequence of SEQ ID NO: 3022.

In another embodiment, the ISVD specifically binding to human PD-1 may have a sequence identity of more than 90%, such as more than 95% or even more than 99%, with SEQ ID NO: 3023, wherein the CDRs are as defined above. In one embodiment, the ISVD specifically binding to PD-1 comprises or consists of the amino acid sequence of SEQ ID NO: 3023.

In another embodiment, the ISVD specifically binding to human PD-1 may have a sequence identity of more than 90%, such as more than 95% or even more than 99%, with SEQ ID NO: 3024, wherein the CDRs are as defined above. In one embodiment, the ISVD specifically binding to PD-1 comprises or consists of the amino acid sequence of SEQ ID NO: 3024.

In another embodiment, the ISVD specifically binding to human PD-1 may have a sequence identity of more than 90%, such as more than 95% or even more than 99%, with SEQ ID NO: 3025, wherein the CDRs are as defined above. In one embodiment, the ISVD specifically binding to PD-1 comprises or consists of the amino acid sequence of SEQ ID NO: 3025.

In another embodiment, the ISVD specifically binding to human PD-1 may have a sequence identity of more than 90%, such as more than 95% or even more than 99%, with SEQ ID NO: 3026, wherein the CDRs are as defined above. In one embodiment, the ISVD specifically binding to PD-1 comprises or consists of the amino acid sequence of SEQ ID NO: 3026.

In another embodiment, the ISVD specifically binding to human PD-1 may have a sequence identity of more than 90%, such as more than 95% or even more than 99%, with SEQ ID NO: 3027, wherein the CDRs are as defined above. In one embodiment, the ISVD specifically binding to PD-1 comprises or consists of the amino acid sequence of SEQ ID NO: 3027.

In another embodiment, the ISVD specifically binding to human PD-1 may have a sequence identity of more than 90%, such as more than 95% or even more than 99%, with SEQ ID NO: 3028, wherein the CDRs are as defined above. In one embodiment, the ISVD specifically binding to PD-1 comprises or consists of the amino acid sequence of SEQ ID NO: 3028.

In another embodiment, the ISVD specifically binding to human PD-1 may have a sequence identity of more than 90%, such as more than 95% or even more than 99%, with SEQ ID NO: 3029, wherein the CDRs are as defined above. In one embodiment, the ISVD specifically binding to PD-1 comprises or consists of the amino acid sequence of SEQ ID NO: 3029.

In another embodiment, the ISVD specifically binding to human PD-1 may have a sequence identity of more than 90%, such as more than 95% or even more than 99%, with SEQ ID NO: 3030, wherein the CDRs are as defined above. In one embodiment, the ISVD specifically binding to PD-1 comprises or consists of the amino acid sequence of SEQ ID NO: 3030.

In another embodiment, the ISVD specifically binding to human PD-1 may have a sequence identity of more than 90%, such as more than 95% or even more than 99%, with SEQ ID NO: 3031, wherein the CDRs are as defined above. In one embodiment, the ISVD specifically binding to PD-1 comprises or consists of the amino acid sequence of SEQ ID NO: 3031.

In another embodiment, the ISVD specifically binding to human PD-1 may have a sequence identity of more than 90%, such as more than 95% or even more than 99%, with SEQ ID NO: 3032, wherein the CDRs are as defined above. In one embodiment, the ISVD specifically binding to PD-1 comprises or consists of the amino acid sequence of SEQ ID NO: 3032.

In another embodiment, the ISVD specifically binding to human PD-1 may have a sequence identity of more than 90%, such as more than 95% or even more than 99%, with SEQ ID NO: 3033, wherein the CDRs are as defined above. In one embodiment, the ISVD specifically binding to PD-1 comprises or consists of the amino acid sequence of SEQ ID NO: 3033.

In another embodiment, the ISVD specifically binding to human PD-1 may have a sequence identity of more than 90%, such as more than 95% or even more than 99%, with SEQ ID NO: 3034, wherein the CDRs are as defined above. In one embodiment, the ISVD specifically binding to PD-1 comprises or consists of the amino acid sequence of SEQ ID NO: 3034.

In another embodiment, the ISVD specifically binding to human PD-1 may have a sequence identity of more than 90%, such as more than 95% or even more than 99%, with SEQ ID NO: 3035, wherein the CDRs are as defined above. In one embodiment, the ISVD specifically binding to PD-1 comprises or consists of the amino acid sequence of SEQ ID NO: 3035.

In another embodiment, the ISVD specifically binding to human PD-1 may have a sequence identity of more than 90%, such as more than 95% or even more than 99%, with SEQ ID NO: 3036, wherein the CDRs are as defined above. In one embodiment, the ISVD specifically binding to PD-1 comprises or consists of the amino acid sequence of SEQ ID NO: 3036.

In another embodiment, the ISVD specifically binding to human PD-1 may have a sequence identity of more than 90%, such as more than 95% or even more than 99%, with SEQ ID NO: 3037, wherein the CDRs are as defined above. In one embodiment, the ISVD specifically binding to PD-1 comprises or consists of the amino acid sequence of SEQ ID NO: 3037.

In another embodiment, the ISVD specifically binding to human PD-1 may have a sequence identity of more than 90%, such as more than 95% or even more than 99%, with SEQ ID NO: 3038, wherein the CDRs are as defined above. In one embodiment, the ISVD specifically binding to PD-1 comprises or consists of the amino acid sequence of SEQ ID NO: 3038.

In another embodiment, the ISVD specifically binding to human PD-1 may have a sequence identity of more than 90%, such as more than 95% or even more than 99%, with SEQ ID NO: 3039, wherein the CDRs are as defined above. In one embodiment, the ISVD specifically binding to PD-1 comprises or consists of the amino acid sequence of SEQ ID NO: 3039.

In another embodiment, the ISVD specifically binding to human PD-1 may have a sequence identity of more than 90%, such as more than 95% or even more than 99%, with SEQ ID NO: 3040, wherein the CDRs are as defined above. In one embodiment, the ISVD specifically binding to PD-1 comprises or consists of the amino acid sequence of SEQ ID NO: 3040.

In another embodiment, the ISVD specifically binding to human PD-1 may have a sequence identity of more than 90%, such as more than 95% or even more than 99%, with SEQ ID NO: 3041, wherein the CDRs are as defined above. In one embodiment, the ISVD specifically binding to PD-1 comprises or consists of the amino acid sequence of SEQ ID NO: 3041.

In another embodiment, the ISVD specifically binding to human PD-1 may have a sequence identity of more than 90%, such as more than 95% or even more than 99%, with SEQ ID NO: 3042, wherein the CDRs are as defined above. In one embodiment, the ISVD specifically binding to PD-1 comprises or consists of the amino acid sequence of SEQ ID NO: 3042.

In another embodiment, the ISVD specifically binding to human PD-1 may have a sequence identity of more than 90%, such as more than 95% or even more than 99%, with SEQ ID NO: 3043, wherein the CDRs are as defined above. In one embodiment, the ISVD specifically binding to PD-1 comprises or consists of the amino acid sequence of SEQ ID NO: 3043.

In another embodiment, the ISVD specifically binding to human PD-1 may have a sequence identity of more than 90%, such as more than 95% or even more than 99%, with SEQ ID NO: 3044, wherein the CDRs are as defined above. In one embodiment, the ISVD specifically binding to PD-1 comprises or consists of the amino acid sequence of SEQ ID NO: 3044.

In another embodiment, the ISVD specifically binding to human PD-1 may have a sequence identity of more than 90%, such as more than 95% or even more than 99%, with SEQ ID NO: 3045, wherein the CDRs are as defined above. In one embodiment, the ISVD specifically binding to PD-1 comprises or consists of the amino acid sequence of SEQ ID NO: 3045.

In another embodiment, the ISVD specifically binding to human PD-1 may have a sequence identity of more than 90%, such as more than 95% or even more than 99%, with SEQ ID NO: 3046, wherein the CDRs are as defined above. In one embodiment, the ISVD specifically binding to PD-1 comprises or consists of the amino acid sequence of SEQ ID NO: 3046.

In another embodiment, the ISVD specifically binding to human PD-1 may have a sequence identity of more than 90%, such as more than 95% or even more than 99%, with SEQ ID NO: 3047, wherein the CDRs are as defined above. In one embodiment, the ISVD specifically binding to PD-1 comprises or consists of the amino acid sequence of SEQ ID NO: 3047.

In another embodiment, the ISVD specifically binding to human PD-1 may have a sequence identity of more than 90%, such as more than 95% or even more than 99%, with SEQ ID NO: 3048, wherein the CDRs are as defined above. In one embodiment, the ISVD specifically binding to PD-1 comprises or consists of the amino acid sequence of SEQ ID NO: 3048.

In another embodiment, the ISVD specifically binding to human PD-1 may have a sequence identity of more than 90%, such as more than 95% or even more than 99%, with SEQ ID NO: 3049, wherein the CDRs are as defined above. In one embodiment, the ISVD specifically binding to PD-1 comprises or consists of the amino acid sequence of SEQ ID NO: 3049.

In another embodiment, the ISVD specifically binding to human PD-1 may have a sequence identity of more than 90%, such as more than 95% or even more than 99%, with SEQ ID NO: 3050, wherein the CDRs are as defined above. In one embodiment, the ISVD specifically binding to PD-1 comprises or consists of the amino acid sequence of SEQ ID NO: 3050.

In another embodiment, the ISVD specifically binding to human PD-1 may have a sequence identity of more than 90%, such as more than 95% or even more than 99%, with SEQ ID NO: 3051, wherein the CDRs are as defined above. In one embodiment, the ISVD specifically binding to PD-1 comprises or consists of the amino acid sequence of SEQ ID NO: 3051.

In another embodiment, the ISVD specifically binding to human PD-1 may have a sequence identity of more than 90%, such as more than 95% or even more than 99%, with SEQ ID NO: 3052, wherein the CDRs are as defined above. In one embodiment, the ISVD specifically binding to PD-1 comprises or consists of the amino acid sequence of SEQ ID NO: 3052.

In another embodiment, the ISVD specifically binding to human PD-1 may have a sequence identity of more than 90%, such as more than 95% or even more than 99%, with SEQ ID NO: 3053, wherein the CDRs are as defined above. In one embodiment, the ISVD specifically binding to PD-1 comprises or consists of the amino acid sequence of SEQ ID NO: 3053.

In another embodiment, the ISVD specifically binding to human PD-1 may have a sequence identity of more than 90%, such as more than 95% or even more than 99%, with SEQ ID NO: 3054, wherein the CDRs are as defined above. In one embodiment, the ISVD specifically binding to PD-1 comprises or consists of the amino acid sequence of SEQ ID NO: 3054.

In another embodiment, the ISVD specifically binding to human PD-1 may have a sequence identity of more than 90%, such as more than 95% or even more than 99%, with SEQ ID NO: 3055, wherein the CDRs are as defined above. In one embodiment, the ISVD specifically binding to PD-1 comprises or consists of the amino acid sequence of SEQ ID NO: 3055.

In another embodiment, the ISVD specifically binding to human PD-1 may have a sequence identity of more than 90%, such as more than 95% or even more than 99%, with SEQ ID NO: 3056, wherein the CDRs are as defined above. In one embodiment, the ISVD specifically binding to PD-1 comprises or consists of the amino acid sequence of SEQ ID NO: 3056.

In another embodiment, the ISVD specifically binding to human PD-1 may have a sequence identity of more than 90%, such as more than 95% or even more than 99%, with SEQ ID NO: 3057, wherein the CDRs are as defined above. In one embodiment, the ISVD specifically binding to PD-1 comprises or consists of the amino acid sequence of SEQ ID NO: 3057.

In another embodiment, the ISVD specifically binding to human PD-1 may have a sequence identity of more than 90%, such as more than 95% or even more than 99%, with SEQ ID NO: 3058, wherein the CDRs are as defined above. In one embodiment, the ISVD specifically binding to PD-1 comprises or consists of the amino acid sequence of SEQ ID NO: 3058.

In another embodiment, the ISVD specifically binding to human PD-1 may have a sequence identity of more than 90%, such as more than 95% or even more than 99%, with SEQ ID NO: 3059, wherein the CDRs are as defined above. In one embodiment, the ISVD specifically binding to PD-1 comprises or consists of the amino acid sequence of SEQ ID NO: 3059.

In another embodiment, the ISVD specifically binding to human PD-1 may have a sequence identity of more than 90%, such as more than 95% or even more than 99%, with SEQ ID NO: 3060, wherein the CDRs are as defined above. In one embodiment, the ISVD specifically binding to PD-1 comprises or consists of the amino acid sequence of SEQ ID NO: 3060.

In another embodiment, the ISVD specifically binding to human PD-1 may have a sequence identity of more than 90%, such as more than 95% or even more than 99%, with SEQ ID NO: 3061, wherein the CDRs are as defined above. In one embodiment, the ISVD specifically binding to PD-1 comprises or consists of the amino acid sequence of SEQ ID NO: 3061.

In another embodiment, the ISVD specifically binding to human PD-1 may have a sequence identity of more than 90%, such as more than 95% or even more than 99%, with SEQ ID NO: 3062, wherein the CDRs are as defined above. In one embodiment, the ISVD specifically binding to PD-1 comprises or consists of the amino acid sequence of SEQ ID NO: 3062.

In another embodiment, the ISVD specifically binding to human PD-1 may have a sequence identity of more than 90%, such as more than 95% or even more than 99%, with SEQ ID NO: 3063, wherein the CDRs are as defined above. In one embodiment, the ISVD specifically binding to PD-1 comprises or consists of the amino acid sequence of SEQ ID NO: 3063.

In another embodiment, the ISVD specifically binding to human PD-1 may have a sequence identity of more than 90%, such as more than 95% or even more than 99%, with SEQ ID NO: 3064, wherein the CDRs are as defined above. In one embodiment, the ISVD specifically binding to PD-1 comprises or consists of the amino acid sequence of SEQ ID NO: 3064.

In another embodiment, the ISVD specifically binding to human PD-1 may have a sequence identity of more than 90%, such as more than 95% or even more than 99%, with SEQ ID NO: 3065, wherein the CDRs are as defined above. In one embodiment, the ISVD specifically binding to PD-1 comprises or consists of the amino acid sequence of SEQ ID NO: 3065.

In another embodiment, the ISVD specifically binding to human PD-1 may have a sequence identity of more than 90%, such as more than 95% or even more than 99%, with SEQ ID NO: 3066, wherein the CDRs are as defined above. In one embodiment, the ISVD specifically binding to PD-1 comprises or consists of the amino acid sequence of SEQ ID NO: 3066.

In another embodiment, the ISVD specifically binding to human PD-1 may have a sequence identity of more than 90%, such as more than 95% or even more than 99%, with SEQ ID NO: 3067, wherein the CDRs are as defined above. In one embodiment, the ISVD specifically binding to PD-1 comprises or consists of the amino acid sequence of SEQ ID NO: 3067.

In another embodiment, the ISVD specifically binding to human PD-1 may have a sequence identity of more than 90%, such as more than 95% or even more than 99%, with SEQ ID NO: 3068, wherein the CDRs are as defined above. In one embodiment, the ISVD specifically binding to PD-1 comprises or consists of the amino acid sequence of SEQ ID NO: 3068.

In another embodiment, the ISVD specifically binding to human PD-1 may have a sequence identity of more than 90%, such as more than 95% or even more than 99%, with SEQ ID NO: 3069, wherein the CDRs are as defined above. In one embodiment, the ISVD specifically binding to PD-1 comprises or consists of the amino acid sequence of SEQ ID NO: 3069.

In another embodiment, the ISVD specifically binding to human PD-1 may have a sequence identity of more than 90%, such as more than 95% or even more than 99%, with SEQ ID NO: 3070, wherein the CDRs are as defined above. In one embodiment, the ISVD specifically binding to PD-1 comprises or consists of the amino acid sequence of SEQ ID NO: 3070.

In another embodiment, the ISVD specifically binding to human PD-1 may have a sequence identity of more than 90%, such as more than 95% or even more than 99%, with SEQ ID NO: 3071, wherein the CDRs are as defined above. In one embodiment, the ISVD specifically binding to PD-1 comprises or consists of the amino acid sequence of SEQ ID NO: 3071.

In another embodiment, the ISVD specifically binding to human PD-1 may have a sequence identity of more than 90%, such as more than 95% or even more than 99%, with SEQ ID NO: 3072, wherein the CDRs are as defined above. In one embodiment, the ISVD specifically binding to PD-1 comprises or consists of the amino acid sequence of SEQ ID NO: 3072.

In another embodiment, the ISVD specifically binding to human PD-1 may have a sequence identity of more than 90%, such as more than 95% or even more than 99%, with SEQ ID NO: 3073, wherein the CDRs are as defined above. In one embodiment, the ISVD specifically binding to PD-1 comprises or consists of the amino acid sequence of SEQ ID NO: 3073.

In another embodiment, the ISVD specifically binding to human PD-1 may have a sequence identity of more than 90%, such as more than 95% or even more than 99%, with SEQ ID NO: 3074, wherein the CDRs are as defined above. In one embodiment, the ISVD specifically binding to PD-1 comprises or consists of the amino acid sequence of SEQ ID NO: 3074.

In another embodiment, the ISVD specifically binding to human PD-1 may have a sequence identity of more than 90%, such as more than 95% or even more than 99%, with SEQ ID NO: 3075, wherein the CDRs are as defined above. In one embodiment, the ISVD specifically binding to PD-1 comprises or consists of the amino acid sequence of SEQ ID NO: 3075.

In another embodiment, the ISVD specifically binding to human PD-1 may have a sequence identity of more than 90%, such as more than 95% or even more than 99%, with SEQ ID NO: 3076, wherein the CDRs are as defined above. In one embodiment, the ISVD specifically binding to PD-1 comprises or consists of the amino acid sequence of SEQ ID NO: 3076.

In another embodiment, the ISVD specifically binding to human PD-1 may have a sequence identity of more than 90%, such as more than 95% or even more than 99%, with SEQ ID NO: 3077, wherein the CDRs are as defined above. In one embodiment, the ISVD specifically binding to PD-1 comprises or consists of the amino acid sequence of SEQ ID NO: 3077.

In another embodiment, the ISVD specifically binding to human PD-1 may have a sequence identity of more than 90%, such as more than 95% or even more than 99%, with SEQ ID NO: 3078, wherein the CDRs are as defined above. In one embodiment, the ISVD specifically binding to PD-1 comprises or consists of the amino acid sequence of SEQ ID NO: 3078.

In another embodiment, the ISVD specifically binding to human PD-1 may have a sequence identity of more than 90%, such as more than 95% or even more than 99%, with SEQ ID NO: 3079, wherein the CDRs are as defined above. In one embodiment, the ISVD specifically binding to PD-1 comprises or consists of the amino acid sequence of SEQ ID NO: 3079.

In another embodiment, the ISVD specifically binding to human PD-1 may have a sequence identity of more than 90%, such as more than 95% or even more than 99%, with SEQ ID NO: 3080, wherein the CDRs are as defined above. In one embodiment, the ISVD specifically binding to PD-1 comprises or consists of the amino acid sequence of SEQ ID NO: 3080.

In another embodiment, the ISVD specifically binding to human PD-1 may have a sequence identity of more than 90%, such as more than 95% or even more than 99%, with SEQ ID NO: 3081, wherein the CDRs are as defined above. In one embodiment, the ISVD specifically binding to PD-1 comprises or consists of the amino acid sequence of SEQ ID NO: 3081.

In another embodiment, the ISVD specifically binding to human PD-1 may have a sequence identity of more than 90%, such as more than 95% or even more than 99%, with SEQ ID NO: 3082, wherein the CDRs are as defined above. In one embodiment, the ISVD specifically binding to PD-1 comprises or consists of the amino acid sequence of SEQ ID NO: 3082.

In another embodiment, the ISVD specifically binding to human PD-1 may have a sequence identity of more than 90%, such as more than 95% or even more than 99%, with SEQ ID NO: 3083, wherein the CDRs are as defined above. In one embodiment, the ISVD specifically binding to PD-1 comprises or consists of the amino acid sequence of SEQ ID NO: 3083.

In another embodiment, the ISVD specifically binding to human PD-1 may have a sequence identity of more than 90%, such as more than 95% or even more than 99%, with SEQ ID NO: 3084, wherein the CDRs are as defined above. In one embodiment, the ISVD specifically binding to PD-1 comprises or consists of the amino acid sequence of SEQ ID NO: 3084.

In another embodiment, the ISVD specifically binding to human PD-1 may have a sequence identity of more than 90%, such as more than 95% or even more than 99%, with SEQ ID NO: 3085, wherein the CDRs are as defined above. In one embodiment, the ISVD specifically binding to PD-1 comprises or consists of the amino acid sequence of SEQ ID NO: 3085.

In another embodiment, the ISVD specifically binding to human PD-1 may have a sequence identity of more than 90%, such as more than 95% or even more than 99%, with SEQ ID NO: 3086, wherein the CDRs are as defined above. In one embodiment, the ISVD specifically binding to PD-1 comprises or consists of the amino acid sequence of SEQ ID NO: 3086.

In another embodiment, the ISVD specifically binding to human PD-1 may have a sequence identity of more than 90%, such as more than 95% or even more than 99%, with SEQ ID NO: 3087, wherein the CDRs are as defined above. In one embodiment, the ISVD specifically binding to PD-1 comprises or consists of the amino acid sequence of SEQ ID NO: 3087.

In another embodiment, the ISVD specifically binding to human PD-1 may have a sequence identity of more than 90%, such as more than 95% or even more than 99%, with SEQ ID NO: 3088, wherein the CDRs are as defined above. In one embodiment, the ISVD specifically binding to PD-1 comprises or consists of the amino acid sequence of SEQ ID NO: 3088.

In another embodiment, the ISVD specifically binding to human PD-1 may have a sequence identity of more than 90%, such as more than 95% or even more than 99%, with SEQ ID NO: 3089, wherein the CDRs are as defined above. In one embodiment, the ISVD specifically binding to PD-1 comprises or consists of the amino acid sequence of SEQ ID NO: 3089.

In another embodiment, the ISVD specifically binding to human PD-1 may have a sequence identity of more than 90%, such as more than 95% or even more than 99%, with SEQ ID NO: 3090, wherein the CDRs are as defined above. In one embodiment, the ISVD specifically binding to PD-1 comprises or consists of the amino acid sequence of SEQ ID NO: 3090.

In another embodiment, the ISVD specifically binding to human PD-1 may have a sequence identity of more than 90%, such as more than 95% or even more than 99%, with SEQ ID NO: 3091, wherein the CDRs are as defined above. In one embodiment, the ISVD specifically binding to PD-1 comprises or consists of the amino acid sequence of SEQ ID NO: 3091.

In another embodiment, the ISVD specifically binding to human PD-1 may have a sequence identity of more than 90%, such as more than 95% or even more than 99%, with SEQ ID NO: 3092, wherein the CDRs are as defined above. In one embodiment, the ISVD specifically binding to PD-1 comprises or consists of the amino acid sequence of SEQ ID NO: 3092.

In another embodiment, the ISVD specifically binding to human PD-1 may have a sequence identity of more than 90%, such as more than 95% or even more than 99%, with SEQ ID NO: 3093, wherein the CDRs are as defined above. In one embodiment, the ISVD specifically binding to PD-1 comprises or consists of the amino acid sequence of SEQ ID NO: 3093.

In another embodiment, the ISVD specifically binding to human PD-1 may have a sequence identity of more than 90%, such as more than 95% or even more than 99%, with SEQ ID NO: 3094, wherein the CDRs are as defined above. In one embodiment, the ISVD specifically binding to PD-1 comprises or consists of the amino acid sequence of SEQ ID NO: 3094.

In another embodiment, the ISVD specifically binding to human PD-1 may have a sequence identity of more than 90%, such as more than 95% or even more than 99%, with SEQ ID NO: 3095, wherein the CDRs are as defined above. In one embodiment, the ISVD specifically binding to PD-1 comprises or consists of the amino acid sequence of SEQ ID NO: 3095.

In another embodiment, the ISVD specifically binding to human PD-1 may have a sequence identity of more than 90%, such as more than 95% or even more than 99%, with SEQ ID NO: 3096, wherein the CDRs are as defined above. In one embodiment, the ISVD specifically binding to PD-1 comprises or consists of the amino acid sequence of SEQ ID NO: 3096.

In another embodiment, the ISVD specifically binding to human PD-1 may have a sequence identity of more than 90%, such as more than 95% or even more than 99%, with SEQ ID NO: 3097, wherein the CDRs are as defined above. In one embodiment, the ISVD specifically binding to PD-1 comprises or consists of the amino acid sequence of SEQ ID NO: 3097.

In another embodiment, the ISVD specifically binding to human PD-1 may have a sequence identity of more than 90%, such as more than 95% or even more than 99%, with SEQ ID NO: 3098, wherein the CDRs are as defined above. In one embodiment, the ISVD specifically binding to PD-1 comprises or consists of the amino acid sequence of SEQ ID NO: 3098.

In another embodiment, the ISVD specifically binding to human PD-1 may have a sequence identity of more than 90%, such as more than 95% or even more than 99%, with SEQ ID NO: 3099, wherein the CDRs are as defined above. In one embodiment, the ISVD specifically binding to PD-1 comprises or consists of the amino acid sequence of SEQ ID NO: 3099.

In another embodiment, the ISVD specifically binding to human PD-1 may have a sequence identity of more than 90%, such as more than 95% or even more than 99%, with SEQ ID NO: 3100, wherein the CDRs are as defined above. In one embodiment, the ISVD specifically binding to PD-1 comprises or consists of the amino acid sequence of SEQ ID NO: 3100.

In another embodiment, the ISVD specifically binding to human PD-1 may have a sequence identity of more than 90%, such as more than 95% or even more than 99%, with SEQ ID NO: 3101, wherein the CDRs are as defined above. In one embodiment, the ISVD specifically binding to PD-1 comprises or consists of the amino acid sequence of SEQ ID NO: 3101.

In another embodiment, the ISVD specifically binding to human PD-1 may have a sequence identity of more than 90%, such as more than 95% or even more than 99%, with SEQ ID NO: 3102, wherein the CDRs are as defined above. In one embodiment, the ISVD specifically binding to PD-1 comprises or consists of the amino acid sequence of SEQ ID NO: 3102.

In another embodiment, the ISVD specifically binding to human PD-1 may have a sequence identity of more than 90%, such as more than 95% or even more than 99%, with SEQ ID NO: 3103, wherein the CDRs are as defined above. In one embodiment, the ISVD specifically binding to PD-1 comprises or consists of the amino acid sequence of SEQ ID NO: 3103.

In another embodiment, the ISVD specifically binding to human PD-1 may have a sequence identity of more than 90%, such as more than 95% or even more than 99%, with SEQ ID NO: 3104, wherein the CDRs are as defined above. In one embodiment, the ISVD specifically binding to PD-1 comprises or consists of the amino acid sequence of SEQ ID NO: 3104.

In another embodiment, the ISVD specifically binding to human PD-1 may have a sequence identity of more than 90%, such as more than 95% or even more than 99%, with SEQ ID NO: 3105, wherein the CDRs are as defined above. In one embodiment, the ISVD specifically binding to PD-1 comprises or consists of the amino acid sequence of SEQ ID NO: 3105.

In another embodiment, the ISVD specifically binding to human PD-1 may have a sequence identity of more than 90%, such as more than 95% or even more than 99%, with SEQ ID NO: 3106, wherein the CDRs are as defined above. In one embodiment, the ISVD specifically binding to PD-1 comprises or consists of the amino acid sequence of SEQ ID NO: 3106.

In another embodiment, the ISVD specifically binding to human PD-1 may have a sequence identity of more than 90%, such as more than 95% or even more than 99%, with SEQ ID NO: 3107, wherein the CDRs are as defined above. In one embodiment, the ISVD specifically binding to PD-1 comprises or consists of the amino acid sequence of SEQ ID NO: 3107.

In another embodiment, the ISVD specifically binding to human PD-1 may have a sequence identity of more than 90%, such as more than 95% or even more than 99%, with SEQ ID NO: 3108, wherein the CDRs are as defined above. In one embodiment, the ISVD specifically binding to PD-1 comprises or consists of the amino acid sequence of SEQ ID NO: 3108.

In another embodiment, the ISVD specifically binding to human PD-1 may have a sequence identity of more than 90%, such as more than 95% or even more than 99%, with SEQ ID NO: 3109, wherein the CDRs are as defined above. In one embodiment, the ISVD specifically binding to PD-1 comprises or consists of the amino acid sequence of SEQ ID NO: 3109.

In another embodiment, the ISVD specifically binding to human PD-1 may have a sequence identity of more than 90%, such as more than 95% or even more than 99%, with SEQ ID NO: 3110, wherein the CDRs are as defined above. In one embodiment, the ISVD specifically binding to PD-1 comprises or consists of the amino acid sequence of SEQ ID NO: 3110.

In another embodiment, the ISVD specifically binding to human PD-1 may have a sequence identity of more than 90%, such as more than 95% or even more than 99%, with SEQ ID NO: 3111, wherein the CDRs are as defined above. In one embodiment, the ISVD specifically binding to PD-1 comprises or consists of the amino acid sequence of SEQ ID NO: 3111.

In another embodiment, the ISVD specifically binding to human PD-1 may have a sequence identity of more than 90%, such as more than 95% or even more than 99%, with SEQ ID NO: 3112, wherein the CDRs are as defined above. In one embodiment, the ISVD specifically binding to PD-1 comprises or consists of the amino acid sequence of SEQ ID NO: 3112.

In another embodiment, the ISVD specifically binding to human PD-1 may have a sequence identity of more than 90%, such as more than 95% or even more than 99%, with SEQ ID NO: 3113, wherein the CDRs are as defined above. In one embodiment, the ISVD specifically binding to PD-1 comprises or consists of the amino acid sequence of SEQ ID NO: 3113.

In another embodiment, the ISVD specifically binding to human PD-1 may have a sequence identity of more than 90%, such as more than 95% or even more than 99%, with SEQ ID NO: 3114, wherein the CDRs are as defined above. In one embodiment, the ISVD specifically binding to PD-1 comprises or consists of the amino acid sequence of SEQ ID NO: 3114.

In another embodiment, the ISVD specifically binding to human PD-1 may have a sequence identity of more than 90%, such as more than 95% or even more than 99%, with SEQ ID NO: 3115, wherein the CDRs are as defined above. In one embodiment, the ISVD specifically binding to PD-1 comprises or consists of the amino acid sequence of SEQ ID NO: 3115.

In another embodiment, the ISVD specifically binding to human PD-1 may have a sequence identity of more than 90%, such as more than 95% or even more than 99%, with SEQ ID NO: 3116, wherein the CDRs are as defined above. In one embodiment, the ISVD specifically binding to PD-1 comprises or consists of the amino acid sequence of SEQ ID NO: 3116.

In another embodiment, the ISVD specifically binding to human PD-1 may have a sequence identity of more than 90%, such as more than 95% or even more than 99%, with SEQ ID NO: 3117, wherein the CDRs are as defined above. In one embodiment, the ISVD specifically binding to PD-1 comprises or consists of the amino acid sequence of SEQ ID NO: 3117.

In another embodiment, the ISVD specifically binding to human PD-1 may have a sequence identity of more than 90%, such as more than 95% or even more than 99%, with SEQ ID NO: 3118, wherein the CDRs are as defined above. In one embodiment, the ISVD specifically binding to PD-1 comprises or consists of the amino acid sequence of SEQ ID NO: 3118.

In another embodiment, the ISVD specifically binding to human PD-1 may have a sequence identity of more than 90%, such as more than 95% or even more than 99%, with SEQ ID NO: 3119, wherein the CDRs are as defined above. In one embodiment, the ISVD specifically binding to PD-1 comprises or consists of the amino acid sequence of SEQ ID NO: 3119.

In another embodiment, the ISVD specifically binding to human PD-1 may have a sequence identity of more than 90%, such as more than 95% or even more than 99%, with SEQ ID NO: 3120, wherein the CDRs are as defined above. In one embodiment, the ISVD specifically binding to PD-1 comprises or consists of the amino acid sequence of SEQ ID NO: 3120.

In another embodiment, the ISVD specifically binding to human PD-1 may have a sequence identity of more than 90%, such as more than 95% or even more than 99%, with SEQ ID NO: 3121, wherein the CDRs are as defined above. In one embodiment, the ISVD specifically binding to PD-1 comprises or consists of the amino acid sequence of SEQ ID NO: 3121.

In another embodiment, the ISVD specifically binding to human PD-1 may have a sequence identity of more than 90%, such as more than 95% or even more than 99%, with SEQ ID NO: 3122, wherein the CDRs are as defined above. In one embodiment, the ISVD specifically binding to PD-1 comprises or consists of the amino acid sequence of SEQ ID NO: 3122.

In another embodiment, the ISVD specifically binding to human PD-1 may have a sequence identity of more than 90%, such as more than 95% or even more than 99%, with SEQ ID NO: 3123, wherein the CDRs are as defined above. In one embodiment, the ISVD specifically binding to PD-1 comprises or consists of the amino acid sequence of SEQ ID NO: 3123.

In another embodiment, the ISVD specifically binding to human PD-1 may have a sequence identity of more than 90%, such as more than 95% or even more than 99%, with SEQ ID NO: 3124, wherein the CDRs are as defined above. In one embodiment, the ISVD specifically binding to PD-1 comprises or consists of the amino acid sequence of SEQ ID NO: 3124.

In another embodiment, the ISVD specifically binding to human PD-1 may have a sequence identity of more than 90%, such as more than 95% or even more than 99%, with SEQ ID NO: 3125, wherein the CDRs are as defined above. In one embodiment, the ISVD specifically binding to PD-1 comprises or consists of the amino acid sequence of SEQ ID NO: 3125.

In another embodiment, the ISVD specifically binding to human PD-1 may have a sequence identity of more than 90%, such as more than 95% or even more than 99%, with SEQ ID NO: 3126, wherein the CDRs are as defined above. In one embodiment, the ISVD specifically binding to PD-1 comprises or consists of the amino acid sequence of SEQ ID NO: 3126.

In another embodiment, the ISVD specifically binding to human PD-1 may have a sequence identity of more than 90%, such as more than 95% or even more than 99%, with SEQ ID NO: 3127, wherein the CDRs are as defined above. In one embodiment, the ISVD specifically binding to PD-1 comprises or consists of the amino acid sequence of SEQ ID NO: 3127.

In another embodiment, the ISVD specifically binding to human PD-1 may have a sequence identity of more than 90%, such as more than 95% or even more than 99%, with SEQ ID NO: 3128, wherein the CDRs are as defined above. In one embodiment, the ISVD specifically binding to PD-1 comprises or consists of the amino acid sequence of SEQ ID NO: 3128.

In another embodiment, the ISVD specifically binding to human PD-1 may have a sequence identity of more than 90%, such as more than 95% or even more than 99%, with SEQ ID NO: 3129, wherein the CDRs are as defined above. In one embodiment, the ISVD specifically binding to PD-1 comprises or consists of the amino acid sequence of SEQ ID NO: 3129.

In another embodiment, the ISVD specifically binding to human PD-1 may have a sequence identity of more than 90%, such as more than 95% or even more than 99%, with SEQ ID NO: 3130, wherein the CDRs are as defined above. In one embodiment, the ISVD specifically binding to PD-1 comprises or consists of the amino acid sequence of SEQ ID NO: 3130.

In another embodiment, the ISVD specifically binding to human PD-1 may have a sequence identity of more than 90%, such as more than 95% or even more than 99%, with SEQ ID NO: 3131, wherein the CDRs are as defined above. In one embodiment, the ISVD specifically binding to PD-1 comprises or consists of the amino acid sequence of SEQ ID NO: 3131.

In another embodiment, the ISVD specifically binding to human PD-1 may have a sequence identity of more than 90%, such as more than 95% or even more than 99%, with SEQ ID NO: 3132, wherein the CDRs are as defined above. In one embodiment, the ISVD specifically binding to PD-1 comprises or consists of the amino acid sequence of SEQ ID NO: 3132.

In another embodiment, the ISVD specifically binding to human PD-1 may have a sequence identity of more than 90%, such as more than 95% or even more than 99%, with SEQ ID NO: 3133, wherein the CDRs are as defined above. In one embodiment, the ISVD specifically binding to PD-1 comprises or consists of the amino acid sequence of SEQ ID NO: 3133.

In another embodiment, the ISVD specifically binding to human PD-1 may have a sequence identity of more than 90%, such as more than 95% or even more than 99%, with SEQ ID NO: 3134, wherein the CDRs are as defined above. In one embodiment, the ISVD specifically binding to PD-1 comprises or consists of the amino acid sequence of SEQ ID NO: 3134.

In another embodiment, the ISVD specifically binding to human PD-1 may have a sequence identity of more than 90%, such as more than 95% or even more than 99%, with SEQ ID NO: 3135, wherein the CDRs are as defined above. In one embodiment, the ISVD specifically binding to PD-1 comprises or consists of the amino acid sequence of SEQ ID NO: 3135.

In another embodiment, the ISVD specifically binding to human PD-1 may have a sequence identity of more than 90%, such as more than 95% or even more than 99%, with SEQ ID NO: 3136, wherein the CDRs are as defined above. In one embodiment, the ISVD specifically binding to PD-1 comprises or consists of the amino acid sequence of SEQ ID NO: 3136.

In another embodiment, the ISVD specifically binding to human PD-1 may have a sequence identity of more than 90%, such as more than 95% or even more than 99%, with SEQ ID NO: 3137, wherein the CDRs are as defined above. In one embodiment, the ISVD specifically binding to PD-1 comprises or consists of the amino acid sequence of SEQ ID NO: 3137.

In another embodiment, the ISVD specifically binding to human PD-1 may have a sequence identity of more than 90%, such as more than 95% or even more than 99%, with SEQ ID NO: 3138, wherein the CDRs are as defined above. In one embodiment, the ISVD specifically binding to PD-1 comprises or consists of the amino acid sequence of SEQ ID NO: 3138.

In another embodiment, the ISVD specifically binding to human PD-1 may have a sequence identity of more than 90%, such as more than 95% or even more than 99%, with SEQ ID NO: 3139, wherein the CDRs are as defined above. In one embodiment, the ISVD specifically binding to PD-1 comprises or consists of the amino acid sequence of SEQ ID NO: 3139.

In another embodiment, the ISVD specifically binding to human PD-1 may have a sequence identity of more than 90%, such as more than 95% or even more than 99%, with SEQ ID NO: 3140, wherein the CDRs are as defined above. In one embodiment, the ISVD specifically binding to PD-1 comprises or consists of the amino acid sequence of SEQ ID NO: 3140.

In another embodiment, the ISVD specifically binding to human PD-1 may have a sequence identity of more than 90%, such as more than 95% or even more than 99%, with SEQ ID NO: 3141, wherein the CDRs are as defined above. In one embodiment, the ISVD specifically binding to PD-1 comprises or consists of the amino acid sequence of SEQ ID NO: 3141.

In another embodiment, the ISVD specifically binding to human PD-1 may have a sequence identity of more than 90%, such as more than 95% or even more than 99%, with SEQ ID NO: 3142, wherein the CDRs are as defined above. In one embodiment, the ISVD specifically binding to PD-1 comprises or consists of the amino acid sequence of SEQ ID NO: 3142.

In another embodiment, the ISVD specifically binding to human PD-1 may have a sequence identity of more than 90%, such as more than 95% or even more than 99%, with SEQ ID NO: 3143, wherein the CDRs are as defined above. In one embodiment, the ISVD specifically binding to PD-1 comprises or consists of the amino acid sequence of SEQ ID NO: 3143.

In another embodiment, the ISVD specifically binding to human PD-1 may have a sequence identity of more than 90%, such as more than 95% or even more than 99%, with SEQ ID NO: 3144, wherein the CDRs are as defined above. In one embodiment, the ISVD specifically binding to PD-1 comprises or consists of the amino acid sequence of SEQ ID NO: 3144.

In another embodiment, the ISVD specifically binding to human PD-1 may have a sequence identity of more than 90%, such as more than 95% or even more than 99%, with SEQ ID NO: 3145, wherein the CDRs are as defined above. In one embodiment, the ISVD specifically binding to PD-1 comprises or consists of the amino acid sequence of SEQ ID NO: 3145.

In another embodiment, the ISVD specifically binding to human PD-1 may have a sequence identity of more than 90%, such as more than 95% or even more than 99%, with SEQ ID NO: 3146, wherein the CDRs are as defined above. In one embodiment, the ISVD specifically binding to PD-1 comprises or consists of the amino acid sequence of SEQ ID NO: 3146.

In another embodiment, the ISVD specifically binding to human PD-1 may have a sequence identity of more than 90%, such as more than 95% or even more than 99%, with SEQ ID NO: 3147, wherein the CDRs are as defined above. In one embodiment, the ISVD specifically binding to PD-1 comprises or consists of the amino acid sequence of SEQ ID NO: 3147.

In another embodiment, the ISVD specifically binding to human PD-1 may have a sequence identity of more than 90%, such as more than 95% or even more than 99%, with SEQ ID NO: 3148, wherein the CDRs are as defined above. In one embodiment, the ISVD specifically binding to PD-1 comprises or consists of the amino acid sequence of SEQ ID NO: 3148.

In another embodiment, the ISVD specifically binding to human PD-1 may have a sequence identity of more than 90%, such as more than 95% or even more than 99%, with SEQ ID NO: 3149, wherein the CDRs are as defined above. In one embodiment, the ISVD specifically binding to PD-1 comprises or consists of the amino acid sequence of SEQ ID NO: 3149.

In another embodiment, the ISVD specifically binding to human PD-1 may have a sequence identity of more than 90%, such as more than 95% or even more than 99%, with SEQ ID NO: 3150, wherein the CDRs are as defined above. In one embodiment, the ISVD specifically binding to PD-1 comprises or consists of the amino acid sequence of SEQ ID NO: 3150.

In another embodiment, the ISVD specifically binding to human PD-1 may have a sequence identity of more than 90%, such as more than 95% or even more than 99%, with SEQ ID NO: 3151, wherein the CDRs are as defined above. In one embodiment, the ISVD specifically binding to PD-1 comprises or consists of the amino acid sequence of SEQ ID NO: 3151.

In another embodiment, the ISVD specifically binding to human PD-1 may have a sequence identity of more than 90%, such as more than 95% or even more than 99%, with SEQ ID NO: 3152, wherein the CDRs are as defined above. In one embodiment, the ISVD specifically binding to PD-1 comprises or consists of the amino acid sequence of SEQ ID NO: 3152.

In another embodiment, the ISVD specifically binding to human PD-1 may have a sequence identity of more than 90%, such as more than 95% or even more than 99%, with SEQ ID NO: 3153, wherein the CDRs are as defined above. In one embodiment, the ISVD specifically binding to PD-1 comprises or consists of the amino acid sequence of SEQ ID NO: 3153.

In another embodiment, the ISVD specifically binding to human PD-1 may have a sequence identity of more than 90%, such as more than 95% or even more than 99%, with SEQ ID NO: 3154, wherein the CDRs are as defined above. In one embodiment, the ISVD specifically binding to PD-1 comprises or consists of the amino acid sequence of SEQ ID NO: 3154.

In another embodiment, the ISVD specifically binding to human PD-1 may have a sequence identity of more than 90%, such as more than 95% or even more than 99%, with SEQ ID NO: 3155, wherein the CDRs are as defined above. In one embodiment, the ISVD specifically binding to PD-1 comprises or consists of the amino acid sequence of SEQ ID NO: 3155.

In another embodiment, the ISVD specifically binding to human PD-1 may have a sequence identity of more than 90%, such as more than 95% or even more than 99%, with SEQ ID NO: 3156, wherein the CDRs are as defined above. In one embodiment, the ISVD specifically binding to PD-1 comprises or consists of the amino acid sequence of SEQ ID NO: 3156.

In another embodiment, the ISVD specifically binding to human PD-1 may have a sequence identity of more than 90%, such as more than 95% or even more than 99%, with SEQ ID NO: 3157, wherein the CDRs are as defined above. In one embodiment, the ISVD specifically binding to PD-1 comprises or consists of the amino acid sequence of SEQ ID NO: 3157.

In another embodiment, the ISVD specifically binding to human PD-1 may have a sequence identity of more than 90%, such as more than 95% or even more than 99%, with SEQ ID NO: 3158, wherein the CDRs are as defined above. In one embodiment, the ISVD specifically binding to PD-1 comprises or consists of the amino acid sequence of SEQ ID NO: 3158.

In another embodiment, the ISVD specifically binding to human PD-1 may have a sequence identity of more than 90%, such as more than 95% or even more than 99%, with SEQ ID NO: 3159, wherein the CDRs are as defined above. In one embodiment, the ISVD specifically binding to PD-1 comprises or consists of the amino acid sequence of SEQ ID NO: 3159.

In another embodiment, the ISVD specifically binding to human PD-1 may have a sequence identity of more than 90%, such as more than 95% or even more than 99%, with SEQ ID NO: 3160, wherein the CDRs are as defined above. In one embodiment, the ISVD specifically binding to PD-1 comprises or consists of the amino acid sequence of SEQ ID NO: 3160.

In another embodiment, the ISVD specifically binding to human PD-1 may have a sequence identity of more than 90%, such as more than 95% or even more than 99%, with SEQ ID NO: 3161, wherein the CDRs are as defined above. In one embodiment, the ISVD specifically binding to PD-1 comprises or consists of the amino acid sequence of SEQ ID NO: 3161.

In another embodiment, the ISVD specifically binding to human PD-1 may have a sequence identity of more than 90%, such as more than 95% or even more than 99%, with SEQ ID NO: 3162, wherein the CDRs are as defined above. In one embodiment, the ISVD specifically binding to PD-1 comprises or consists of the amino acid sequence of SEQ ID NO: 3162.

In another embodiment, the ISVD specifically binding to human PD-1 may have a sequence identity of more than 90%, such as more than 95% or even more than 99%, with SEQ ID NO: 3163, wherein the CDRs are as defined above. In one embodiment, the ISVD specifically binding to PD-1 comprises or consists of the amino acid sequence of SEQ ID NO: 3163.

In another embodiment, the ISVD specifically binding to human PD-1 may have a sequence identity of more than 90%, such as more than 95% or even more than 99%, with SEQ ID NO: 3164, wherein the CDRs are as defined above. In one embodiment, the ISVD specifically binding to PD-1 comprises or consists of the amino acid sequence of SEQ ID NO: 3164.

In another embodiment, the ISVD specifically binding to human PD-1 may have a sequence identity of more than 90%, such as more than 95% or even more than 99%, with SEQ ID NO: 3165, wherein the CDRs are as defined above. In one embodiment, the ISVD specifically binding to PD-1 comprises or consists of the amino acid sequence of SEQ ID NO: 3165.

In another embodiment, the ISVD specifically binding to human PD-1 may have a sequence identity of more than 90%, such as more than 95% or even more than 99%, with SEQ ID NO: 3166, wherein the CDRs are as defined above. In one embodiment, the ISVD specifically binding to PD-1 comprises or consists of the amino acid sequence of SEQ ID NO: 3166.

In another embodiment, the ISVD specifically binding to human PD-1 may have a sequence identity of more than 90%, such as more than 95% or even more than 99%, with SEQ ID NO: 3167, wherein the CDRs are as defined above. In one embodiment, the ISVD specifically binding to PD-1 comprises or consists of the amino acid sequence of SEQ ID NO: 3167.

In another embodiment, the ISVD specifically binding to human PD-1 may have a sequence identity of more than 90%, such as more than 95% or even more than 99%, with SEQ ID NO: 3168, wherein the CDRs are as defined above. In one embodiment, the ISVD specifically binding to PD-1 comprises or consists of the amino acid sequence of SEQ ID NO: 3168.

In another embodiment, the ISVD specifically binding to human PD-1 may have a sequence identity of more than 90%, such as more than 95% or even more than 99%, with SEQ ID NO: 3169, wherein the CDRs are as defined above. In one embodiment, the ISVD specifically binding to PD-1 comprises or consists of the amino acid sequence of SEQ ID NO: 3169.

In another embodiment, the ISVD specifically binding to human PD-1 may have a sequence identity of more than 90%, such as more than 95% or even more than 99%, with SEQ ID NO: 3170, wherein the CDRs are as defined above. In one embodiment, the ISVD specifically binding to PD-1 comprises or consists of the amino acid sequence of SEQ ID NO: 3170.

In another embodiment, the ISVD specifically binding to human PD-1 may have a sequence identity of more than 90%, such as more than 95% or even more than 99%, with SEQ ID NO: 3171, wherein the CDRs are as defined above. In one embodiment, the ISVD specifically binding to PD-1 comprises or consists of the amino acid sequence of SEQ ID NO: 3171.

In another embodiment, the ISVD specifically binding to human PD-1 may have a sequence identity of more than 90%, such as more than 95% or even more than 99%, with SEQ ID NO: 3172, wherein the CDRs are as defined above. In one embodiment, the ISVD specifically binding to PD-1 comprises or consists of the amino acid sequence of SEQ ID NO: 3172.

In another embodiment, the ISVD specifically binding to human PD-1 may have a sequence identity of more than 90%, such as more than 95% or even more than 99%, with SEQ ID NO: 3173, wherein the CDRs are as defined above. In one embodiment, the ISVD specifically binding to PD-1 comprises or consists of the amino acid sequence of SEQ ID NO: 3173.

In another embodiment, the ISVD specifically binding to human PD-1 may have a sequence identity of more than 90%, such as more than 95% or even more than 99%, with SEQ ID NO: 3174, wherein the CDRs are as defined above. In one embodiment, the ISVD specifically binding to PD-1 comprises or consists of the amino acid sequence of SEQ ID NO: 3174.

In another embodiment, the ISVD specifically binding to human PD-1 may have a sequence identity of more than 90%, such as more than 95% or even more than 99%, with SEQ ID NO: 3175, wherein the CDRs are as defined above. In one embodiment, the ISVD specifically binding to PD-1 comprises or consists of the amino acid sequence of SEQ ID NO: 3175.

In another embodiment, the ISVD specifically binding to human PD-1 may have a sequence identity of more than 90%, such as more than 95% or even more than 99%, with SEQ ID NO: 3176, wherein the CDRs are as defined above. In one embodiment, the ISVD specifically binding to PD-1 comprises or consists of the amino acid sequence of SEQ ID NO: 3176.

In another embodiment, the ISVD specifically binding to human PD-1 may have a sequence identity of more than 90%, such as more than 95% or even more than 99%, with SEQ ID NO: 3177, wherein the CDRs are as defined above. In one embodiment, the ISVD specifically binding to PD-1 comprises or consists of the amino acid sequence of SEQ ID NO: 3177.

In another embodiment, the ISVD specifically binding to human PD-1 may have a sequence identity of more than 90%, such as more than 95% or even more than 99%, with SEQ ID NO: 3178, wherein the CDRs are as defined above. In one embodiment, the ISVD specifically binding to PD-1 comprises or consists of the amino acid sequence of SEQ ID NO: 3178.

In another embodiment, the ISVD specifically binding to human PD-1 may have a sequence identity of more than 90%, such as more than 95% or even more than 99%, with SEQ ID NO: 3179, wherein the CDRs are as defined above. In one embodiment, the ISVD specifically binding to PD-1 comprises or consists of the amino acid sequence of SEQ ID NO: 3179.

In another embodiment, the ISVD specifically binding to human PD-1 may have a sequence identity of more than 90%, such as more than 95% or even more than 99%, with SEQ ID NO: 3180, wherein the CDRs are as defined above. In one embodiment, the ISVD specifically binding to PD-1 comprises or consists of the amino acid sequence of SEQ ID NO: 3180.

In another embodiment, the ISVD specifically binding to human PD-1 may have a sequence identity of more than 90%, such as more than 95% or even more than 99%, with SEQ ID NO: 3181, wherein the CDRs are as defined above. In one embodiment, the ISVD specifically binding to PD-1 comprises or consists of the amino acid sequence of SEQ ID NO: 3181.

In another embodiment, the ISVD specifically binding to human PD-1 may have a sequence identity of more than 90%, such as more than 95% or even more than 99%, with SEQ ID NO: 3182, wherein the CDRs are as defined above. In one embodiment, the ISVD specifically binding to PD-1 comprises or consists of the amino acid sequence of SEQ ID NO: 3182.

In another embodiment, the ISVD specifically binding to human PD-1 may have a sequence identity of more than 90%, such as more than 95% or even more than 99%, with SEQ ID NO: 3183, wherein the CDRs are as defined above. In one embodiment, the ISVD specifically binding to PD-1 comprises or consists of the amino acid sequence of SEQ ID NO: 3183.

In another embodiment, the ISVD specifically binding to human PD-1 may have a sequence identity of more than 90%, such as more than 95% or even more than 99%, with SEQ ID NO: 3184, wherein the CDRs are as defined above. In one embodiment, the ISVD specifically binding to PD-1 comprises or consists of the amino acid sequence of SEQ ID NO: 3184.

In another embodiment, the ISVD specifically binding to human PD-1 may have a sequence identity of more than 90%, such as more than 95% or even more than 99%, with SEQ ID NO: 3185, wherein the CDRs are as defined above. In one embodiment, the ISVD specifically binding to PD-1 comprises or consists of the amino acid sequence of SEQ ID NO: 3185.

In another embodiment, the ISVD specifically binding to human PD-1 may have a sequence identity of more than 90%, such as more than 95% or even more than 99%, with SEQ ID NO: 3186, wherein the CDRs are as defined above. In one embodiment, the ISVD specifically binding to PD-1 comprises or consists of the amino acid sequence of SEQ ID NO: 3186.

In another embodiment, the ISVD specifically binding to human PD-1 may have a sequence identity of more than 90%, such as more than 95% or even more than 99%, with SEQ ID NO: 3187, wherein the CDRs are as defined above. In one embodiment, the ISVD specifically binding to PD-1 comprises or consists of the amino acid sequence of SEQ ID NO: 3187.

In another embodiment, the ISVD specifically binding to human PD-1 may have a sequence identity of more than 90%, such as more than 95% or even more than 99%, with SEQ ID NO: 3188, wherein the CDRs are as defined above. In one embodiment, the ISVD specifically binding to PD-1 comprises or consists of the amino acid sequence of SEQ ID NO: 3188.

In another embodiment, the ISVD specifically binding to human PD-1 may have a sequence identity of more than 90%, such as more than 95% or even more than 99%, with SEQ ID NO: 3189, wherein the CDRs are as defined above. In one embodiment, the ISVD specifically binding to PD-1 comprises or consists of the amino acid sequence of SEQ ID NO: 3189.

In another embodiment, the ISVD specifically binding to human PD-1 may have a sequence identity of more than 90%, such as more than 95% or even more than 99%, with SEQ ID NO: 3190, wherein the CDRs are as defined above. In one embodiment, the ISVD specifically binding to PD-1 comprises or consists of the amino acid sequence of SEQ ID NO: 3190.

In another embodiment, the ISVD specifically binding to human PD-1 may have a sequence identity of more than 90%, such as more than 95% or even more than 99%, with SEQ ID NO: 3191, wherein the CDRs are as defined above. In one embodiment, the ISVD specifically binding to PD-1 comprises or consists of the amino acid sequence of SEQ ID NO: 3191.

In another embodiment, the ISVD specifically binding to human PD-1 may have a sequence identity of more than 90%, such as more than 95% or even more than 99%, with SEQ ID NO: 3192, wherein the CDRs are as defined above. In one embodiment, the ISVD specifically binding to PD-1 comprises or consists of the amino acid sequence of SEQ ID NO: 3192.

In another embodiment, the ISVD specifically binding to human PD-1 may have a sequence identity of more than 90%, such as more than 95% or even more than 99%, with SEQ ID NO: 3193, wherein the CDRs are as defined above. In one embodiment, the ISVD specifically binding to PD-1 comprises or consists of the amino acid sequence of SEQ ID NO: 3193.

In another embodiment, the ISVD specifically binding to human PD-1 may have a sequence identity of more than 90%, such as more than 95% or even more than 99%, with SEQ ID NO: 3194, wherein the CDRs are as defined above. In one embodiment, the ISVD specifically binding to PD-1 comprises or consists of the amino acid sequence of SEQ ID NO: 3194.

In another embodiment, the ISVD specifically binding to human PD-1 may have a sequence identity of more than 90%, such as more than 95% or even more than 99%, with SEQ ID NO: 3195, wherein the CDRs are as defined above. In one embodiment, the ISVD specifically binding to PD-1 comprises or consists of the amino acid sequence of SEQ ID NO: 3195.

In another embodiment, the ISVD specifically binding to human PD-1 may have a sequence identity of more than 90%, such as more than 95% or even more than 99%, with SEQ ID NO: 3196, wherein the CDRs are as defined above. In one embodiment, the ISVD specifically binding to PD-1 comprises or consists of the amino acid sequence of SEQ ID NO: 3196.

In another embodiment, the ISVD specifically binding to human PD-1 may have a sequence identity of more than 90%, such as more than 95% or even more than 99%, with SEQ ID NO: 3197, wherein the CDRs are as defined above. In one embodiment, the ISVD specifically binding to PD-1 comprises or consists of the amino acid sequence of SEQ ID NO: 3197.

In another embodiment, the ISVD specifically binding to human PD-1 may have a sequence identity of more than 90%, such as more than 95% or even more than 99%, with SEQ ID NO: 3198, wherein the CDRs are as defined above. In one embodiment, the ISVD specifically binding to PD-1 comprises or consists of the amino acid sequence of SEQ ID NO: 3198.

In another embodiment, the ISVD specifically binding to human PD-1 may have a sequence identity of more than 90%, such as more than 95% or even more than 99%, with SEQ ID NO: 3199, wherein the CDRs are as defined above. In one embodiment, the ISVD specifically binding to PD-1 comprises or consists of the amino acid sequence of SEQ ID NO: 3199.

In another embodiment, the ISVD specifically binding to human PD-1 may have a sequence identity of more than 90%, such as more than 95% or even more than 99%, with SEQ ID NO: 3200, wherein the CDRs are as defined above. In one embodiment, the ISVD specifically binding to PD-1 comprises or consists of the amino acid sequence of SEQ ID NO: 3200.

In another embodiment, the ISVD specifically binding to human PD-1 may have a sequence identity of more than 90%, such as more than 95% or even more than 99%, with SEQ ID NO: 3201, wherein the CDRs are as defined above. In one embodiment, the ISVD specifically binding to PD-1 comprises or consists of the amino acid sequence of SEQ ID NO: 3201.

In another embodiment, the ISVD specifically binding to human PD-1 may have a sequence identity of more than 90%, such as more than 95% or even more than 99%, with SEQ ID NO: 3202, wherein the CDRs are as defined above. In one embodiment, the ISVD specifically binding to PD-1 comprises or consists of the amino acid sequence of SEQ ID NO: 3202.

In another embodiment, the ISVD specifically binding to human PD-1 may have a sequence identity of more than 90%, such as more than 95% or even more than 99%, with SEQ ID NO: 3203, wherein the CDRs are as defined above. In one embodiment, the ISVD specifically binding to PD-1 comprises or consists of the amino acid sequence of SEQ ID NO: 3203.

In another embodiment, the ISVD specifically binding to human PD-1 may have a sequence identity of more than 90%, such as more than 95% or even more than 99%, with SEQ ID NO: 3204, wherein the CDRs are as defined above. In one embodiment, the ISVD specifically binding to PD-1 comprises or consists of the amino acid sequence of SEQ ID NO: 3204.

In another embodiment, the ISVD specifically binding to human PD-1 may have a sequence identity of more than 90%, such as more than 95% or even more than 99%, with SEQ ID NO: 3205, wherein the CDRs are as defined above. In one embodiment, the ISVD specifically binding to PD-1 comprises or consists of the amino acid sequence of SEQ ID NO: 3205.

In another embodiment, the ISVD specifically binding to human PD-1 may have a sequence identity of more than 90%, such as more than 95% or even more than 99%, with SEQ ID NO: 3206, wherein the CDRs are as defined above. In one embodiment, the ISVD specifically binding to PD-1 comprises or consists of the amino acid sequence of SEQ ID NO: 3206.

In another embodiment, the ISVD specifically binding to human PD-1 may have a sequence identity of more than 90%, such as more than 95% or even more than 99%, with SEQ ID NO: 3207, wherein the CDRs are as defined above. In one embodiment, the ISVD specifically binding to PD-1 comprises or consists of the amino acid sequence of SEQ ID NO: 3207.

In another embodiment, the ISVD specifically binding to human PD-1 may have a sequence identity of more than 90%, such as more than 95% or even more than 99%, with SEQ ID NO: 3208, wherein the CDRs are as defined above. In one embodiment, the ISVD specifically binding to PD-1 comprises or consists of the amino acid sequence of SEQ ID NO: 3208.

In another embodiment, the ISVD specifically binding to human PD-1 may have a sequence identity of more than 90%, such as more than 95% or even more than 99%, with SEQ ID NO: 3209, wherein the CDRs are as defined above. In one embodiment, the ISVD specifically binding to PD-1 comprises or consists of the amino acid sequence of SEQ ID NO: 3209.

In another embodiment, the ISVD specifically binding to human PD-1 may have a sequence identity of more than 90%, such as more than 95% or even more than 99%, with SEQ ID NO: 3210, wherein the CDRs are as defined above. In one embodiment, the ISVD specifically binding to PD-1 comprises or consists of the amino acid sequence of SEQ ID NO: 3210.

In another embodiment, the ISVD specifically binding to human PD-1 may have a sequence identity of more than 90%, such as more than 95% or even more than 99%, with SEQ ID NO: 3211, wherein the CDRs are as defined above. In one embodiment, the ISVD specifically binding to PD-1 comprises or consists of the amino acid sequence of SEQ ID NO: 3211.

In another embodiment, the ISVD specifically binding to human PD-1 may have a sequence identity of more than 90%, such as more than 95% or even more than 99%, with SEQ ID NO: 3212, wherein the CDRs are as defined above. In one embodiment, the ISVD specifically binding to PD-1 comprises or consists of the amino acid sequence of SEQ ID NO: 3212.

In another embodiment, the ISVD specifically binding to human PD-1 may have a sequence identity of more than 90%, such as more than 95% or even more than 99%, with SEQ ID NO: 3213, wherein the CDRs are as defined above. In one embodiment, the ISVD specifically binding to PD-1 comprises or consists of the amino acid sequence of SEQ ID NO: 3213.

In another embodiment, the ISVD specifically binding to human PD-1 may have a sequence identity of more than 90%, such as more than 95% or even more than 99%, with SEQ ID NO: 3214, wherein the CDRs are as defined above. In one embodiment, the ISVD specifically binding to PD-1 comprises or consists of the amino acid sequence of SEQ ID NO: 3214.

In another embodiment, the ISVD specifically binding to human PD-1 may have a sequence identity of more than 90%, such as more than 95% or even more than 99%, with SEQ ID NO: 3215, wherein the CDRs are as defined above. In one embodiment, the ISVD specifically binding to PD-1 comprises or consists of the amino acid sequence of SEQ ID NO: 3215.

In another embodiment, the ISVD specifically binding to human PD-1 may have a sequence identity of more than 90%, such as more than 95% or even more than 99%, with SEQ ID NO: 3216, wherein the CDRs are as defined above. In one embodiment, the ISVD specifically binding to PD-1 comprises or consists of the amino acid sequence of SEQ ID NO: 3216.

In another embodiment, the ISVD specifically binding to human PD-1 may have a sequence identity of more than 90%, such as more than 95% or even more than 99%, with SEQ ID NO: 3217, wherein the CDRs are as defined above. In one embodiment, the ISVD specifically binding to PD-1 comprises or consists of the amino acid sequence of SEQ ID NO: 3217.

In another embodiment, the ISVD specifically binding to human PD-1 may have a sequence identity of more than 90%, such as more than 95% or even more than 99%, with SEQ ID NO: 3218, wherein the CDRs are as defined above. In one embodiment, the ISVD specifically binding to PD-1 comprises or consists of the amino acid sequence of SEQ ID NO: 3218.

In another embodiment, the ISVD specifically binding to human PD-1 may have a sequence identity of more than 90%, such as more than 95% or even more than 99%, with SEQ ID NO: 3219, wherein the CDRs are as defined above. In one embodiment, the ISVD specifically binding to PD-1 comprises or consists of the amino acid sequence of SEQ ID NO: 3219.

In another embodiment, the ISVD specifically binding to human PD-1 may have a sequence identity of more than 90%, such as more than 95% or even more than 99%, with SEQ ID NO: 3220, wherein the CDRs are as defined above. In one embodiment, the ISVD specifically binding to PD-1 comprises or consists of the amino acid sequence of SEQ ID NO: 3220.

In another embodiment, the ISVD specifically binding to human PD-1 may have a sequence identity of more than 90%, such as more than 95% or even more than 99%, with SEQ ID NO: 3221, wherein the CDRs are as defined above. In one embodiment, the ISVD specifically binding to PD-1 comprises or consists of the amino acid sequence of SEQ ID NO: 3221.

In another embodiment, the ISVD specifically binding to human PD-1 may have a sequence identity of more than 90%, such as more than 95% or even more than 99%, with SEQ ID NO: 3222, wherein the CDRs are as defined above. In one embodiment, the ISVD specifically binding to PD-1 comprises or consists of the amino acid sequence of SEQ ID NO: 3222.

In another embodiment, the ISVD specifically binding to human PD-1 may have a sequence identity of more than 90%, such as more than 95% or even more than 99%, with SEQ ID NO: 3223, wherein the CDRs are as defined above. In one embodiment, the ISVD specifically binding to PD-1 comprises or consists of the amino acid sequence of SEQ ID NO: 3223.

In another embodiment, the ISVD specifically binding to human PD-1 may have a sequence identity of more than 90%, such as more than 95% or even more than 99%, with SEQ ID NO: 3224, wherein the CDRs are as defined above. In one embodiment, the ISVD specifically binding to PD-1 comprises or consists of the amino acid sequence of SEQ ID NO: 3224.

In another embodiment, the ISVD specifically binding to human PD-1 may have a sequence identity of more than 90%, such as more than 95% or even more than 99%, with SEQ ID NO: 3225, wherein the CDRs are as defined above. In one embodiment, the ISVD specifically binding to PD-1 comprises or consists of the amino acid sequence of SEQ ID NO: 3225.

In another embodiment, the ISVD specifically binding to human PD-1 may have a sequence identity of more than 90%, such as more than 95% or even more than 99%, with SEQ ID NO: 3226, wherein the CDRs are as defined above. In one embodiment, the ISVD specifically binding to PD-1 comprises or consists of the amino acid sequence of SEQ ID NO: 3226.

In another embodiment, the ISVD specifically binding to human PD-1 may have a sequence identity of more than 90%, such as more than 95% or even more than 99%, with SEQ ID NO: 3227, wherein the CDRs are as defined above. In one embodiment, the ISVD specifically binding to PD-1 comprises or consists of the amino acid sequence of SEQ ID NO: 3227.

In another embodiment, the ISVD specifically binding to human PD-1 may have a sequence identity of more than 90%, such as more than 95% or even more than 99%, with SEQ ID NO: 3228, wherein the CDRs are as defined above. In one embodiment, the ISVD specifically binding to PD-1 comprises or consists of the amino acid sequence of SEQ ID NO: 3228.

In another embodiment, the ISVD specifically binding to human PD-1 may have a sequence identity of more than 90%, such as more than 95% or even more than 99%, with SEQ ID NO: 3229, wherein the CDRs are as defined above. In one embodiment, the ISVD specifically binding to PD-1 comprises or consists of the amino acid sequence of SEQ ID NO: 3229.

In another embodiment, the ISVD specifically binding to human PD-1 may have a sequence identity of more than 90%, such as more than 95% or even more than 99%, with SEQ ID NO: 3230, wherein the CDRs are as defined above. In one embodiment, the ISVD specifically binding to PD-1 comprises or consists of the amino acid sequence of SEQ ID NO: 3230.

In another embodiment, the ISVD specifically binding to human PD-1 may have a sequence identity of more than 90%, such as more than 95% or even more than 99%, with SEQ ID NO: 3231, wherein the CDRs are as defined above. In one embodiment, the ISVD specifically binding to PD-1 comprises or consists of the amino acid sequence of SEQ ID NO: 3231.

In another embodiment, the ISVD specifically binding to human PD-1 may have a sequence identity of more than 90%, such as more than 95% or even more than 99%, with SEQ ID NO: 3232, wherein the CDRs are as defined above. In one embodiment, the ISVD specifically binding to PD-1 comprises or consists of the amino acid sequence of SEQ ID NO: 3232.

In another embodiment, the ISVD specifically binding to human PD-1 may have a sequence identity of more than 90%, such as more than 95% or even more than 99%, with SEQ ID NO: 3233, wherein the CDRs are as defined above. In one embodiment, the ISVD specifically binding to PD-1 comprises or consists of the amino acid sequence of SEQ ID NO: 3233.

In another embodiment, the ISVD specifically binding to human PD-1 may have a sequence identity of more than 90%, such as more than 95% or even more than 99%, with SEQ ID NO: 3234, wherein the CDRs are as defined above. In one embodiment, the ISVD specifically binding to PD-1 comprises or consists of the amino acid sequence of SEQ ID NO: 3234.

In another embodiment, the ISVD specifically binding to human PD-1 may have a sequence identity of more than 90%, such as more than 95% or even more than 99%, with SEQ ID NO: 3235, wherein the CDRs are as defined above. In one embodiment, the ISVD specifically binding to PD-1 comprises or consists of the amino acid sequence of SEQ ID NO: 3235.

In another embodiment, the ISVD specifically binding to human PD-1 may have a sequence identity of more than 90%, such as more than 95% or even more than 99%, with SEQ ID NO: 3236, wherein the CDRs are as defined above. In one embodiment, the ISVD specifically binding to PD-1 comprises or consists of the amino acid sequence of SEQ ID NO: 3236.

In another embodiment, the ISVD specifically binding to human PD-1 may have a sequence identity of more than 90%, such as more than 95% or even more than 99%, with SEQ ID NO: 3237, wherein the CDRs are as defined above. In one embodiment, the ISVD specifically binding to PD-1 comprises or consists of the amino acid sequence of SEQ ID NO: 3237.

In another embodiment, the ISVD specifically binding to human PD-1 may have a sequence identity of more than 90%, such as more than 95% or even more than 99%, with SEQ ID NO: 3238, wherein the CDRs are as defined above. In one embodiment, the ISVD specifically binding to PD-1 comprises or consists of the amino acid sequence of SEQ ID NO: 3238.

In another embodiment, the ISVD specifically binding to human PD-1 may have a sequence identity of more than 90%, such as more than 95% or even more than 99%, with SEQ ID NO: 3239, wherein the CDRs are as defined above. In one embodiment, the ISVD specifically binding to PD-1 comprises or consists of the amino acid sequence of SEQ ID NO: 3239.

In another embodiment, the ISVD specifically binding to human PD-1 may have a sequence identity of more than 90%, such as more than 95% or even more than 99%, with SEQ ID NO: 3240, wherein the CDRs are as defined above. In one embodiment, the ISVD specifically binding to PD-1 comprises or consists of the amino acid sequence of SEQ ID NO: 3240.

In another embodiment, the ISVD specifically binding to human PD-1 may have a sequence identity of more than 90%, such as more than 95% or even more than 99%, with SEQ ID NO: 3241, wherein the CDRs are as defined above. In one embodiment, the ISVD specifically binding to PD-1 comprises or consists of the amino acid sequence of SEQ ID NO: 3241.

In another embodiment, the ISVD specifically binding to human PD-1 may have a sequence identity of more than 90%, such as more than 95% or even more than 99%, with SEQ ID NO: 3242, wherein the CDRs are as defined above. In one embodiment, the ISVD specifically binding to PD-1 comprises or consists of the amino acid sequence of SEQ ID NO: 3242.

In another embodiment, the ISVD specifically binding to human PD-1 may have a sequence identity of more than 90%, such as more than 95% or even more than 99%, with SEQ ID NO: 3243, wherein the CDRs are as defined above. In one embodiment, the ISVD specifically binding to PD-1 comprises or consists of the amino acid sequence of SEQ ID NO: 3243.

In another embodiment, the ISVD specifically binding to human PD-1 may have a sequence identity of more than 90%, such as more than 95% or even more than 99%, with SEQ ID NO: 3244, wherein the CDRs are as defined above. In one embodiment, the ISVD specifically binding to PD-1 comprises or consists of the amino acid sequence of SEQ ID NO: 3244.

In another embodiment, the ISVD specifically binding to human PD-1 may have a sequence identity of more than 90%, such as more than 95% or even more than 99%, with SEQ ID NO: 3245, wherein the CDRs are as defined above. In one embodiment, the ISVD specifically binding to PD-1 comprises or consists of the amino acid sequence of SEQ ID NO: 3245.

In another embodiment, the ISVD specifically binding to human PD-1 may have a sequence identity of more than 90%, such as more than 95% or even more than 99%, with SEQ ID NO: 3246, wherein the CDRs are as defined above. In one embodiment, the ISVD specifically binding to PD-1 comprises or consists of the amino acid sequence of SEQ ID NO: 3246.

In another embodiment, the ISVD specifically binding to human PD-1 may have a sequence identity of more than 90%, such as more than 95% or even more than 99%, with SEQ ID NO: 3247, wherein the CDRs are as defined above. In one embodiment, the ISVD specifically binding to PD-1 comprises or consists of the amino acid sequence of SEQ ID NO: 3247.

In another embodiment, the ISVD specifically binding to human PD-1 may have a sequence identity of more than 90%, such as more than 95% or even more than 99%, with SEQ ID NO: 3248, wherein the CDRs are as defined above. In one embodiment, the ISVD specifically binding to PD-1 comprises or consists of the amino acid sequence of SEQ ID NO: 3248.

In another embodiment, the ISVD specifically binding to human PD-1 may have a sequence identity of more than 90%, such as more than 95% or even more than 99%, with SEQ ID NO: 3249, wherein the CDRs are as defined above. In one embodiment, the ISVD specifically binding to PD-1 comprises or consists of the amino acid sequence of SEQ ID NO: 3249.

In another embodiment, the ISVD specifically binding to human PD-1 may have a sequence identity of more than 90%, such as more than 95% or even more than 99%, with SEQ ID NO: 3250, wherein the CDRs are as defined above. In one embodiment, the ISVD specifically binding to PD-1 comprises or consists of the amino acid sequence of SEQ ID NO: 3250.

In another embodiment, the ISVD specifically binding to human PD-1 may have a sequence identity of more than 90%, such as more than 95% or even more than 99%, with SEQ ID NO: 3251, wherein the CDRs are as defined above. In one embodiment, the ISVD specifically binding to PD-1 comprises or consists of the amino acid sequence of SEQ ID NO: 3251.

In another embodiment, the ISVD specifically binding to human PD-1 may have a sequence identity of more than 90%, such as more than 95% or even more than 99%, with SEQ ID NO: 3252, wherein the CDRs are as defined above. In one embodiment, the ISVD specifically binding to PD-1 comprises or consists of the amino acid sequence of SEQ ID NO: 3252.

In another embodiment, the ISVD specifically binding to human PD-1 may have a sequence identity of more than 90%, such as more than 95% or even more than 99%, with SEQ ID NO: 3253, wherein the CDRs are as defined above. In one embodiment, the ISVD specifically binding to PD-1 comprises or consists of the amino acid sequence of SEQ ID NO: 3253.

In another embodiment, the ISVD specifically binding to human PD-1 may have a sequence identity of more than 90%, such as more than 95% or even more than 99%, with SEQ ID NO: 3254, wherein the CDRs are as defined above. In one embodiment, the ISVD specifically binding to PD-1 comprises or consists of the amino acid sequence of SEQ ID NO: 3254.

In another embodiment, the ISVD specifically binding to human PD-1 may have a sequence identity of more than 90%, such as more than 95% or even more than 99%, with SEQ ID NO: 3255, wherein the CDRs are as defined above. In one embodiment, the ISVD specifically binding to PD-1 comprises or consists of the amino acid sequence of SEQ ID NO: 3255.

In another embodiment, the ISVD specifically binding to human PD-1 may have a sequence identity of more than 90%, such as more than 95% or even more than 99%, with SEQ ID NO: 3256, wherein the CDRs are as defined above. In one embodiment, the ISVD specifically binding to PD-1 comprises or consists of the amino acid sequence of SEQ ID NO: 3256.

In another embodiment, the ISVD specifically binding to human PD-1 may have a sequence identity of more than 90%, such as more than 95% or even more than 99%, with SEQ ID NO: 3257, wherein the CDRs are as defined above. In one embodiment, the ISVD specifically binding to PD-1 comprises or consists of the amino acid sequence of SEQ ID NO: 3257.

In another embodiment, the ISVD specifically binding to human PD-1 may have a sequence identity of more than 90%, such as more than 95% or even more than 99%, with SEQ ID NO: 3258, wherein the CDRs are as defined above. In one embodiment, the ISVD specifically binding to PD-1 comprises or consists of the amino acid sequence of SEQ ID NO: 3258.

In another embodiment, the ISVD specifically binding to human PD-1 may have a sequence identity of more than 90%, such as more than 95% or even more than 99%, with SEQ ID NO: 3259, wherein the CDRs are as defined above. In one embodiment, the ISVD specifically binding to PD-1 comprises or consists of the amino acid sequence of SEQ ID NO: 3259.

In another embodiment, the ISVD specifically binding to human PD-1 may have a sequence identity of more than 90%, such as more than 95% or even more than 99%, with SEQ ID NO: 3260, wherein the CDRs are as defined above. In one embodiment, the ISVD specifically binding to PD-1 comprises or consists of the amino acid sequence of SEQ ID NO: 3260.

In another embodiment, the ISVD specifically binding to human PD-1 may have a sequence identity of more than 90%, such as more than 95% or even more than 99%, with SEQ ID NO: 3261, wherein the CDRs are as defined above. In one embodiment, the ISVD specifically binding to PD-1 comprises or consists of the amino acid sequence of SEQ ID NO: 3261.

In another embodiment, the ISVD specifically binding to human PD-1 may have a sequence identity of more than 90%, such as more than 95% or even more than 99%, with SEQ ID NO: 3262, wherein the CDRs are as defined above. In one embodiment, the ISVD specifically binding to PD-1 comprises or consists of the amino acid sequence of SEQ ID NO: 3262.

In another embodiment, the ISVD specifically binding to human PD-1 may have a sequence identity of more than 90%, such as more than 95% or even more than 99%, with SEQ ID NO: 3263, wherein the CDRs are as defined above. In one embodiment, the ISVD specifically binding to PD-1 comprises or consists of the amino acid sequence of SEQ ID NO: 3263.

In another embodiment, the ISVD specifically binding to human PD-1 may have a sequence identity of more than 90%, such as more than 95% or even more than 99%, with SEQ ID NO: 3264, wherein the CDRs are as defined above. In one embodiment, the ISVD specifically binding to PD-1 comprises or consists of the amino acid sequence of SEQ ID NO: 3264.

In another embodiment, the ISVD specifically binding to human PD-1 may have a sequence identity of more than 90%, such as more than 95% or even more than 99%, with SEQ ID NO: 3265, wherein the CDRs are as defined above. In one embodiment, the ISVD specifically binding to PD-1 comprises or consists of the amino acid sequence of SEQ ID NO: 3265.

In another embodiment, the ISVD specifically binding to human PD-1 may have a sequence identity of more than 90%, such as more than 95% or even more than 99%, with SEQ ID NO: 3266, wherein the CDRs are as defined above. In one embodiment, the ISVD specifically binding to PD-1 comprises or consists of the amino acid sequence of SEQ ID NO: 3266.

In another embodiment, the ISVD specifically binding to human PD-1 may have a sequence identity of more than 90%, such as more than 95% or even more than 99%, with SEQ ID NO: 3267, wherein the CDRs are as defined above. In one embodiment, the ISVD specifically binding to PD-1 comprises or consists of the amino acid sequence of SEQ ID NO: 3267.

In another embodiment, the ISVD specifically binding to human PD-1 may have a sequence identity of more than 90%, such as more than 95% or even more than 99%, with SEQ ID NO: 3268, wherein the CDRs are as defined above. In one embodiment, the ISVD specifically binding to PD-1 comprises or consists of the amino acid sequence of SEQ ID NO: 3268.

In another embodiment, the ISVD specifically binding to human PD-1 may have a sequence identity of more than 90%, such as more than 95% or even more than 99%, with SEQ ID NO: 3269, wherein the CDRs are as defined above. In one embodiment, the ISVD specifically binding to PD-1 comprises or consists of the amino acid sequence of SEQ ID NO: 3269.

In another embodiment, the ISVD specifically binding to human PD-1 may have a sequence identity of more than 90%, such as more than 95% or even more than 99%, with SEQ ID NO: 3270, wherein the CDRs are as defined above. In one embodiment, the ISVD specifically binding to PD-1 comprises or consists of the amino acid sequence of SEQ ID NO: 3270.

In another embodiment, the ISVD specifically binding to human PD-1 may have a sequence identity of more than 90%, such as more than 95% or even more than 99%, with SEQ ID NO: 3271, wherein the CDRs are as defined above. In one embodiment, the ISVD specifically binding to PD-1 comprises or consists of the amino acid sequence of SEQ ID NO: 3271.

In another embodiment, the ISVD specifically binding to human PD-1 may have a sequence identity of more than 90%, such as more than 95% or even more than 99%, with SEQ ID NO: 3272, wherein the CDRs are as defined above. In one embodiment, the ISVD specifically binding to PD-1 comprises or consists of the amino acid sequence of SEQ ID NO: 3272.

In another embodiment, the ISVD specifically binding to human PD-1 may have a sequence identity of more than 90%, such as more than 95% or even more than 99%, with SEQ ID NO: 3273, wherein the CDRs are as defined above. In one embodiment, the ISVD specifically binding to PD-1 comprises or consists of the amino acid sequence of SEQ ID NO: 3273.

In another embodiment, the ISVD specifically binding to human PD-1 may have a sequence identity of more than 90%, such as more than 95% or even more than 99%, with SEQ ID NO: 3274, wherein the CDRs are as defined above. In one embodiment, the ISVD specifically binding to PD-1 comprises or consists of the amino acid sequence of SEQ ID NO: 3274.

In another embodiment, the ISVD specifically binding to human PD-1 may have a sequence identity of more than 90%, such as more than 95% or even more than 99%, with SEQ ID NO: 3275, wherein the CDRs are as defined above. In one embodiment, the ISVD specifically binding to PD-1 comprises or consists of the amino acid sequence of SEQ ID NO: 3275.

In another embodiment, the ISVD specifically binding to human PD-1 may have a sequence identity of more than 90%, such as more than 95% or even more than 99%, with SEQ ID NO: 3276, wherein the CDRs are as defined above. In one embodiment, the ISVD specifically binding to PD-1 comprises or consists of the amino acid sequence of SEQ ID NO: 3276.

In another embodiment, the ISVD specifically binding to human PD-1 may have a sequence identity of more than 90%, such as more than 95% or even more than 99%, with SEQ ID NO: 3277, wherein the CDRs are as defined above. In one embodiment, the ISVD specifically binding to PD-1 comprises or consists of the amino acid sequence of SEQ ID NO: 3277.

In another embodiment, the ISVD specifically binding to human PD-1 may have a sequence identity of more than 90%, such as more than 95% or even more than 99%, with SEQ ID NO: 3278, wherein the CDRs are as defined above. In one embodiment, the ISVD specifically binding to PD-1 comprises or consists of the amino acid sequence of SEQ ID NO: 3278.

In another embodiment, the ISVD specifically binding to human PD-1 may have a sequence identity of more than 90%, such as more than 95% or even more than 99%, with SEQ ID NO: 3279, wherein the CDRs are as defined above. In one embodiment, the ISVD specifically binding to PD-1 comprises or consists of the amino acid sequence of SEQ ID NO: 3279.

In another embodiment, the ISVD specifically binding to human PD-1 may have a sequence identity of more than 90%, such as more than 95% or even more than 99%, with SEQ ID NO: 3280, wherein the CDRs are as defined above. In one embodiment, the ISVD specifically binding to PD-1 comprises or consists of the amino acid sequence of SEQ ID NO: 3280.

In another embodiment, the ISVD specifically binding to human PD-1 may have a sequence identity of more than 90%, such as more than 95% or even more than 99%, with SEQ ID NO: 3281, wherein the CDRs are as defined above. In one embodiment, the ISVD specifically binding to PD-1 comprises or consists of the amino acid sequence of SEQ ID NO: 3281.

In another embodiment, the ISVD specifically binding to human PD-1 may have a sequence identity of more than 90%, such as more than 95% or even more than 99%, with SEQ ID NO: 3282, wherein the CDRs are as defined above. In one embodiment, the ISVD specifically binding to PD-1 comprises or consists of the amino acid sequence of SEQ ID NO: 3282.

In another embodiment, the ISVD specifically binding to human PD-1 may have a sequence identity of more than 90%, such as more than 95% or even more than 99%, with SEQ ID NO: 3283, wherein the CDRs are as defined above. In one embodiment, the ISVD specifically binding to PD-1 comprises or consists of the amino acid sequence of SEQ ID NO: 3283.

In another embodiment, the ISVD specifically binding to human PD-1 may have a sequence identity of more than 90%, such as more than 95% or even more than 99%, with SEQ ID NO: 3284, wherein the CDRs are as defined above. In one embodiment, the ISVD specifically binding to PD-1 comprises or consists of the amino acid sequence of SEQ ID NO: 3284.

In another embodiment, the ISVD specifically binding to human PD-1 may have a sequence identity of more than 90%, such as more than 95% or even more than 99%, with SEQ ID NO: 3285, wherein the CDRs are as defined above. In one embodiment, the ISVD specifically binding to PD-1 comprises or consists of the amino acid sequence of SEQ ID NO: 3285.

In another embodiment, the ISVD specifically binding to human PD-1 may have a sequence identity of more than 90%, such as more than 95% or even more than 99%, with SEQ ID NO: 3286, wherein the CDRs are as defined above. In one embodiment, the ISVD specifically binding to PD-1 comprises or consists of the amino acid sequence of SEQ ID NO: 3286.

In another embodiment, the ISVD specifically binding to human PD-1 may have a sequence identity of more than 90%, such as more than 95% or even more than 99%, with SEQ ID NO: 3287, wherein the CDRs are as defined above. In one embodiment, the ISVD specifically binding to PD-1 comprises or consists of the amino acid sequence of SEQ ID NO: 3287.

In another embodiment, the ISVD specifically binding to human PD-1 may have a sequence identity of more than 90%, such as more than 95% or even more than 99%, with SEQ ID NO: 3288, wherein the CDRs are as defined above. In one embodiment, the ISVD specifically binding to PD-1 comprises or consists of the amino acid sequence of SEQ ID NO: 3288.

In another embodiment, the ISVD specifically binding to human PD-1 may have a sequence identity of more than 90%, such as more than 95% or even more than 99%, with SEQ ID NO: 3289, wherein the CDRs are as defined above. In one embodiment, the ISVD specifically binding to PD-1 comprises or consists of the amino acid sequence of SEQ ID NO: 3289.

In another embodiment, the ISVD specifically binding to human PD-1 may have a sequence identity of more than 90%, such as more than 95% or even more than 99%, with SEQ ID NO: 3290, wherein the CDRs are as defined above. In one embodiment, the ISVD specifically binding to PD-1 comprises or consists of the amino acid sequence of SEQ ID NO: 3290.

In another embodiment, the ISVD specifically binding to human PD-1 may have a sequence identity of more than 90%, such as more than 95% or even more than 99%, with SEQ ID NO: 3291, wherein the CDRs are as defined above. In one embodiment, the ISVD specifically binding to PD-1 comprises or consists of the amino acid sequence of SEQ ID NO: 3291.

In another embodiment, the ISVD specifically binding to human PD-1 may have a sequence identity of more than 90%, such as more than 95% or even more than 99%, with SEQ ID NO: 3292, wherein the CDRs are as defined above. In one embodiment, the ISVD specifically binding to PD-1 comprises or consists of the amino acid sequence of SEQ ID NO: 3292.

In another embodiment, the ISVD specifically binding to human PD-1 may have a sequence identity of more than 90%, such as more than 95% or even more than 99%, with SEQ ID NO: 3293, wherein the CDRs are as defined above. In one embodiment, the ISVD specifically binding to PD-1 comprises or consists of the amino acid sequence of SEQ ID NO: 3293.

In another embodiment, the ISVD specifically binding to human PD-1 may have a sequence identity of more than 90%, such as more than 95% or even more than 99%, with SEQ ID NO: 3294, wherein the CDRs are as defined above. In one embodiment, the ISVD specifically binding to PD-1 comprises or consists of the amino acid sequence of SEQ ID NO: 3294.

In another embodiment, the ISVD specifically binding to human PD-1 may have a sequence identity of more than 90%, such as more than 95% or even more than 99%, with SEQ ID NO: 3295, wherein the CDRs are as defined above. In one embodiment, the ISVD specifically binding to PD-1 comprises or consists of the amino acid sequence of SEQ ID NO: 3295.

In another embodiment, the ISVD specifically binding to human PD-1 may have a sequence identity of more than 90%, such as more than 95% or even more than 99%, with SEQ ID NO: 3296, wherein the CDRs are as defined above. In one embodiment, the ISVD specifically binding to PD-1 comprises or consists of the amino acid sequence of SEQ ID NO: 3296.

In another embodiment, the ISVD specifically binding to human PD-1 may have a sequence identity of more than 90%, such as more than 95% or even more than 99%, with SEQ ID NO: 3297, wherein the CDRs are as defined above. In one embodiment, the ISVD specifically binding to PD-1 comprises or consists of the amino acid sequence of SEQ ID NO: 3297.

In another embodiment, the ISVD specifically binding to human PD-1 may have a sequence identity of more than 90%, such as more than 95% or even more than 99%, with SEQ ID NO: 3298, wherein the CDRs are as defined above. In one embodiment, the ISVD specifically binding to PD-1 comprises or consists of the amino acid sequence of SEQ ID NO: 3298.

In another embodiment, the ISVD specifically binding to human PD-1 may have a sequence identity of more than 90%, such as more than 95% or even more than 99%, with SEQ ID NO: 3299, wherein the CDRs are as defined above. In one embodiment, the ISVD specifically binding to PD-1 comprises or consists of the amino acid sequence of SEQ ID NO: 3299.

In another embodiment, the ISVD specifically binding to human PD-1 may have a sequence identity of more than 90%, such as more than 95% or even more than 99%, with SEQ ID NO: 3300, wherein the CDRs are as defined above. In one embodiment, the ISVD specifically binding to PD-1 comprises or consists of the amino acid sequence of SEQ ID NO: 3300.

In another embodiment, the ISVD specifically binding to human PD-1 may have a sequence identity of more than 90%, such as more than 95% or even more than 99%, with SEQ ID NO: 3301, wherein the CDRs are as defined above. In one embodiment, the ISVD specifically binding to PD-1 comprises or consists of the amino acid sequence of SEQ ID NO: 3301.

In another embodiment, the ISVD specifically binding to human PD-1 may have a sequence identity of more than 90%, such as more than 95% or even more than 99%, with SEQ ID NO: 3302, wherein the CDRs are as defined above. In one embodiment, the ISVD specifically binding to PD-1 comprises or consists of the amino acid sequence of SEQ ID NO: 3302.

In another embodiment, the ISVD specifically binding to human PD-1 may have a sequence identity of more than 90%, such as more than 95% or even more than 99%, with SEQ ID NO: 3303, wherein the CDRs are as defined above. In one embodiment, the ISVD specifically binding to PD-1 comprises or consists of the amino acid sequence of SEQ ID NO: 3303.

In another embodiment, the ISVD specifically binding to human PD-1 may have a sequence identity of more than 90%, such as more than 95% or even more than 99%, with SEQ ID NO: 3304, wherein the CDRs are as defined above. In one embodiment, the ISVD specifically binding to PD-1 comprises or consists of the amino acid sequence of SEQ ID NO: 3304.

In another embodiment, the ISVD specifically binding to human PD-1 may have a sequence identity of more than 90%, such as more than 95% or even more than 99%, with SEQ ID NO: 3305, wherein the CDRs are as defined above. In one embodiment, the ISVD specifically binding to PD-1 comprises or consists of the amino acid sequence of SEQ ID NO: 3305.

In another embodiment, the ISVD specifically binding to human PD-1 may have a sequence identity of more than 90%, such as more than 95% or even more than 99%, with SEQ ID NO: 3306, wherein the CDRs are as defined above. In one embodiment, the ISVD specifically binding to PD-1 comprises or consists of the amino acid sequence of SEQ ID NO: 3306.

In another embodiment, the ISVD specifically binding to human PD-1 may have a sequence identity of more than 90%, such as more than 95% or even more than 99%, with SEQ ID NO: 3307, wherein the CDRs are as defined above. In one embodiment, the ISVD specifically binding to PD-1 comprises or consists of the amino acid sequence of SEQ ID NO: 3307.

In another embodiment, the ISVD specifically binding to human PD-1 may have a sequence identity of more than 90%, such as more than 95% or even more than 99%, with SEQ ID NO: 3308, wherein the CDRs are as defined above. In one embodiment, the ISVD specifically binding to PD-1 comprises or consists of the amino acid sequence of SEQ ID NO: 3308.

In another embodiment, the ISVD specifically binding to human PD-1 may have a sequence identity of more than 90%, such as more than 95% or even more than 99%, with SEQ ID NO: 3309, wherein the CDRs are as defined above. In one embodiment, the ISVD specifically binding to PD-1 comprises or consists of the amino acid sequence of SEQ ID NO: 3309.

In another embodiment, the ISVD specifically binding to human PD-1 may have a sequence identity of more than 90%, such as more than 95% or even more than 99%, with SEQ ID NO: 3310, wherein the CDRs are as defined above. In one embodiment, the ISVD specifically binding to PD-1 comprises or consists of the amino acid sequence of SEQ ID NO: 3310.

In another embodiment, the ISVD specifically binding to human PD-1 may have a sequence identity of more than 90%, such as more than 95% or even more than 99%, with SEQ ID NO: 3311, wherein the CDRs are as defined above. In one embodiment, the ISVD specifically binding to PD-1 comprises or consists of the amino acid sequence of SEQ ID NO: 3311.

In another embodiment, the ISVD specifically binding to human PD-1 may have a sequence identity of more than 90%, such as more than 95% or even more than 99%, with SEQ ID NO: 3312, wherein the CDRs are as defined above. In one embodiment, the ISVD specifically binding to PD-1 comprises or consists of the amino acid sequence of SEQ ID NO: 3312.

In another embodiment, the ISVD specifically binding to human PD-1 may have a sequence identity of more than 90%, such as more than 95% or even more than 99%, with SEQ ID NO: 3313, wherein the CDRs are as defined above. In one embodiment, the ISVD specifically binding to PD-1 comprises or consists of the amino acid sequence of SEQ ID NO: 3313.

In another embodiment, the ISVD specifically binding to human PD-1 may have a sequence identity of more than 90%, such as more than 95% or even more than 99%, with SEQ ID NO: 3314, wherein the CDRs are as defined above. In one embodiment, the ISVD specifically binding to PD-1 comprises or consists of the amino acid sequence of SEQ ID NO: 3314.

In another embodiment, the ISVD specifically binding to human PD-1 may have a sequence identity of more than 90%, such as more than 95% or even more than 99%, with SEQ ID NO: 3315, wherein the CDRs are as defined above. In one embodiment, the ISVD specifically binding to PD-1 comprises or consists of the amino acid sequence of SEQ ID NO: 3315.

In another embodiment, the ISVD specifically binding to human PD-1 may have a sequence identity of more than 90%, such as more than 95% or even more than 99%, with SEQ ID NO: 3316, wherein the CDRs are as defined above. In one embodiment, the ISVD specifically binding to PD-1 comprises or consists of the amino acid sequence of SEQ ID NO: 3316.

In another embodiment, the ISVD specifically binding to human PD-1 may have a sequence identity of more than 90%, such as more than 95% or even more than 99%, with SEQ ID NO: 3317, wherein the CDRs are as defined above. In one embodiment, the ISVD specifically binding to PD-1 comprises or consists of the amino acid sequence of SEQ ID NO: 3317.

In another embodiment, the ISVD specifically binding to human PD-1 may have a sequence identity of more than 90%, such as more than 95% or even more than 99%, with SEQ ID NO: 3318, wherein the CDRs are as defined above. In one embodiment, the ISVD specifically binding to PD-1 comprises or consists of the amino acid sequence of SEQ ID NO: 3318.

In another embodiment, the ISVD specifically binding to human PD-1 may have a sequence identity of more than 90%, such as more than 95% or even more than 99%, with SEQ ID NO: 3319, wherein the CDRs are as defined above. In one embodiment, the ISVD specifically binding to PD-1 comprises or consists of the amino acid sequence of SEQ ID NO: 3319.

In another embodiment, the ISVD specifically binding to human PD-1 may have a sequence identity of more than 90%, such as more than 95% or even more than 99%, with SEQ ID NO: 3320, wherein the CDRs are as defined above. In one embodiment, the ISVD specifically binding to PD-1 comprises or consists of the amino acid sequence of SEQ ID NO: 3320.

In another embodiment, the ISVD specifically binding to human PD-1 may have a sequence identity of more than 90%, such as more than 95% or even more than 99%, with SEQ ID NO: 3321, wherein the CDRs are as defined above. In one embodiment, the ISVD specifically binding to PD-1 comprises or consists of the amino acid sequence of SEQ ID NO: 3321.

In another embodiment, the ISVD specifically binding to human PD-1 may have a sequence identity of more than 90%, such as more than 95% or even more than 99%, with SEQ ID NO: 3322, wherein the CDRs are as defined above. In one embodiment, the ISVD specifically binding to PD-1 comprises or consists of the amino acid sequence of SEQ ID NO: 3322.

In another embodiment, the ISVD specifically binding to human PD-1 may have a sequence identity of more than 90%, such as more than 95% or even more than 99%, with SEQ ID NO: 3323, wherein the CDRs are as defined above. In one embodiment, the ISVD specifically binding to PD-1 comprises or consists of the amino acid sequence of SEQ ID NO: 3323.

In another embodiment, the ISVD specifically binding to human PD-1 may have a sequence identity of more than 90%, such as more than 95% or even more than 99%, with SEQ ID NO: 3324, wherein the CDRs are as defined above. In one embodiment, the ISVD specifically binding to PD-1 comprises or consists of the amino acid sequence of SEQ ID NO: 3324.

In another embodiment, the ISVD specifically binding to human PD-1 may have a sequence identity of more than 90%, such as more than 95% or even more than 99%, with SEQ ID NO: 3325, wherein the CDRs are as defined above. In one embodiment, the ISVD specifically binding to PD-1 comprises or consists of the amino acid sequence of SEQ ID NO: 3325.

In another embodiment, the ISVD specifically binding to human PD-1 may have a sequence identity of more than 90%, such as more than 95% or even more than 99%, with SEQ ID NO: 3326, wherein the CDRs are as defined above. In one embodiment, the ISVD specifically binding to PD-1 comprises or consists of the amino acid sequence of SEQ ID NO: 3326.

In another embodiment, the ISVD specifically binding to human PD-1 may have a sequence identity of more than 90%, such as more than 95% or even more than 99%, with SEQ ID NO: 3327, wherein the CDRs are as defined above. In one embodiment, the ISVD specifically binding to PD-1 comprises or consists of the amino acid sequence of SEQ ID NO: 3327.

In another embodiment, the ISVD specifically binding to human PD-1 may have a sequence identity of more than 90%, such as more than 95% or even more than 99%, with SEQ ID NO: 3328, wherein the CDRs are as defined above. In one embodiment, the ISVD specifically binding to PD-1 comprises or consists of the amino acid sequence of SEQ ID NO: 3328.

In another embodiment, the ISVD specifically binding to human PD-1 may have a sequence identity of more than 90%, such as more than 95% or even more than 99%, with SEQ ID NO: 3329, wherein the CDRs are as defined above. In one embodiment, the ISVD specifically binding to PD-1 comprises or consists of the amino acid sequence of SEQ ID NO: 3329.

In another embodiment, the ISVD specifically binding to human PD-1 may have a sequence identity of more than 90%, such as more than 95% or even more than 99%, with SEQ ID NO: 3330, wherein the CDRs are as defined above. In one embodiment, the ISVD specifically binding to PD-1 comprises or consists of the amino acid sequence of SEQ ID NO: 3330.

In another embodiment, the ISVD specifically binding to human PD-1 may have a sequence identity of more than 90%, such as more than 95% or even more than 99%, with SEQ ID NO: 3331, wherein the CDRs are as defined above. In one embodiment, the ISVD specifically binding to PD-1 comprises or consists of the amino acid sequence of SEQ ID NO: 3331.

In another embodiment, the ISVD specifically binding to human PD-1 may have a sequence identity of more than 90%, such as more than 95% or even more than 99%, with SEQ ID NO: 3332, wherein the CDRs are as defined above. In one embodiment, the ISVD specifically binding to PD-1 comprises or consists of the amino acid sequence of SEQ ID NO: 3332.

In another embodiment, the ISVD specifically binding to human PD-1 may have a sequence identity of more than 90%, such as more than 95% or even more than 99%, with SEQ ID NO: 3333, wherein the CDRs are as defined above. In one embodiment, the ISVD specifically binding to PD-1 comprises or consists of the amino acid sequence of SEQ ID NO: 3333.

In another embodiment, the ISVD specifically binding to human PD-1 may have a sequence identity of more than 90%, such as more than 95% or even more than 99%, with SEQ ID NO: 3334, wherein the CDRs are as defined above. In one embodiment, the ISVD specifically binding to PD-1 comprises or consists of the amino acid sequence of SEQ ID NO: 3334.

In another embodiment, the ISVD specifically binding to human PD-1 may have a sequence identity of more than 90%, such as more than 95% or even more than 99%, with SEQ ID NO: 3335, wherein the CDRs are as defined above. In one embodiment, the ISVD specifically binding to PD-1 comprises or consists of the amino acid sequence of SEQ ID NO: 3335.

In another embodiment, the ISVD specifically binding to human PD-1 may have a sequence identity of more than 90%, such as more than 95% or even more than 99%, with SEQ ID NO: 3336, wherein the CDRs are as defined above. In one embodiment, the ISVD specifically binding to PD-1 comprises or consists of the amino acid sequence of SEQ ID NO: 3336.

In another embodiment, the ISVD specifically binding to human PD-1 may have a sequence identity of more than 90%, such as more than 95% or even more than 99%, with SEQ ID NO: 3337, wherein the CDRs are as defined above. In one embodiment, the ISVD specifically binding to PD-1 comprises or consists of the amino acid sequence of SEQ ID NO: 3337.

In another embodiment, the ISVD specifically binding to human PD-1 may have a sequence identity of more than 90%, such as more than 95% or even more than 99%, with SEQ ID NO: 3338, wherein the CDRs are as defined above. In one embodiment, the ISVD specifically binding to PD-1 comprises or consists of the amino acid sequence of SEQ ID NO: 3338.

In another embodiment, the ISVD specifically binding to human PD-1 may have a sequence identity of more than 90%, such as more than 95% or even more than 99%, with SEQ ID NO: 3339, wherein the CDRs are as defined above. In one embodiment, the ISVD specifically binding to PD-1 comprises or consists of the amino acid sequence of SEQ ID NO: 3339.

In another embodiment, the ISVD specifically binding to human PD-1 may have a sequence identity of more than 90%, such as more than 95% or even more than 99%, with SEQ ID NO: 3340, wherein the CDRs are as defined above. In one embodiment, the ISVD specifically binding to PD-1 comprises or consists of the amino acid sequence of SEQ ID NO: 3340.

In another embodiment, the ISVD specifically binding to human PD-1 may have a sequence identity of more than 90%, such as more than 95% or even more than 99%, with SEQ ID NO: 3341, wherein the CDRs are as defined above. In one embodiment, the ISVD specifically binding to PD-1 comprises or consists of the amino acid sequence of SEQ ID NO: 3341.

In another embodiment, the ISVD specifically binding to human PD-1 may have a sequence identity of more than 90%, such as more than 95% or even more than 99%, with SEQ ID NO: 3342, wherein the CDRs are as defined above. In one embodiment, the ISVD specifically binding to PD-1 comprises or consists of the amino acid sequence of SEQ ID NO: 3342.

In another embodiment, the ISVD specifically binding to human PD-1 may have a sequence identity of more than 90%, such as more than 95% or even more than 99%, with SEQ ID NO: 3343, wherein the CDRs are as defined above. In one embodiment, the ISVD specifically binding to PD-1 comprises or consists of the amino acid sequence of SEQ ID NO: 3343.

In another embodiment, the ISVD specifically binding to human PD-1 may have a sequence identity of more than 90%, such as more than 95% or even more than 99%, with SEQ ID NO: 3344, wherein the CDRs are as defined above. In one embodiment, the ISVD specifically binding to PD-1 comprises or consists of the amino acid sequence of SEQ ID NO: 3344.

In another embodiment, the ISVD specifically binding to human PD-1 may have a sequence identity of more than 90%, such as more than 95% or even more than 99%, with SEQ ID NO: 3345, wherein the CDRs are as defined above. In one embodiment, the ISVD specifically binding to PD-1 comprises or consists of the amino acid sequence of SEQ ID NO: 3345.

In another embodiment, the ISVD specifically binding to human PD-1 may have a sequence identity of more than 90%, such as more than 95% or even more than 99%, with SEQ ID NO: 3346, wherein the CDRs are as defined above. In one embodiment, the ISVD specifically binding to PD-1 comprises or consists of the amino acid sequence of SEQ ID NO: 3346.

In another embodiment, the ISVD specifically binding to human PD-1 may have a sequence identity of more than 90%, such as more than 95% or even more than 99%, with SEQ ID NO: 3347, wherein the CDRs are as defined above. In one embodiment, the ISVD specifically binding to PD-1 comprises or consists of the amino acid sequence of SEQ ID NO: 3347.

In another embodiment, the ISVD specifically binding to human PD-1 may have a sequence identity of more than 90%, such as more than 95% or even more than 99%, with SEQ ID NO: 3348, wherein the CDRs are as defined above. In one embodiment, the ISVD specifically binding to PD-1 comprises or consists of the amino acid sequence of SEQ ID NO: 3348.

In another embodiment, the ISVD specifically binding to human PD-1 may have a sequence identity of more than 90%, such as more than 95% or even more than 99%, with SEQ ID NO: 3349, wherein the CDRs are as defined above. In one embodiment, the ISVD specifically binding to PD-1 comprises or consists of the amino acid sequence of SEQ ID NO: 3349.

In another embodiment, the ISVD specifically binding to human PD-1 may have a sequence identity of more than 90%, such as more than 95% or even more than 99%, with SEQ ID NO: 3350, wherein the CDRs are as defined above. In one embodiment, the ISVD specifically binding to PD-1 comprises or consists of the amino acid sequence of SEQ ID NO: 3350.

In another embodiment, the ISVD specifically binding to human PD-1 may have a sequence identity of more than 90%, such as more than 95% or even more than 99%, with SEQ ID NO: 3351, wherein the CDRs are as defined above. In one embodiment, the ISVD specifically binding to PD-1 comprises or consists of the amino acid sequence of SEQ ID NO: 3351.

In another embodiment, the ISVD specifically binding to human PD-1 may have a sequence identity of more than 90%, such as more than 95% or even more than 99%, with SEQ ID NO: 3352, wherein the CDRs are as defined above. In one embodiment, the ISVD specifically binding to PD-1 comprises or consists of the amino acid sequence of SEQ ID NO: 3352.

In another embodiment, the ISVD specifically binding to human PD-1 may have a sequence identity of more than 90%, such as more than 95% or even more than 99%, with SEQ ID NO: 3353, wherein the CDRs are as defined above. In one embodiment, the ISVD specifically binding to PD-1 comprises or consists of the amino acid sequence of SEQ ID NO: 3353.

In another embodiment, the ISVD specifically binding to human PD-1 may have a sequence identity of more than 90%, such as more than 95% or even more than 99%, with SEQ ID NO: 3354, wherein the CDRs are as defined above. In one embodiment, the ISVD specifically binding to PD-1 comprises or consists of the amino acid sequence of SEQ ID NO: 3354.

In another embodiment, the ISVD specifically binding to human PD-1 may have a sequence identity of more than 90%, such as more than 95% or even more than 99%, with SEQ ID NO: 3355, wherein the CDRs are as defined above. In one embodiment, the ISVD specifically binding to PD-1 comprises or consists of the amino acid sequence of SEQ ID NO: 3355.

In another embodiment, the ISVD specifically binding to human PD-1 may have a sequence identity of more than 90%, such as more than 95% or even more than 99%, with SEQ ID NO: 3356, wherein the CDRs are as defined above. In one embodiment, the ISVD specifically binding to PD-1 comprises or consists of the amino acid sequence of SEQ ID NO: 3356.

In another embodiment, the ISVD specifically binding to human PD-1 may have a sequence identity of more than 90%, such as more than 95% or even more than 99%, with SEQ ID NO: 3357, wherein the CDRs are as defined above. In one embodiment, the ISVD specifically binding to PD-1 comprises or consists of the amino acid sequence of SEQ ID NO: 3357.

In another embodiment, the ISVD specifically binding to human PD-1 may have a sequence identity of more than 90%, such as more than 95% or even more than 99%, with SEQ ID NO: 3358, wherein the CDRs are as defined above. In one embodiment, the ISVD specifically binding to PD-1 comprises or consists of the amino acid sequence of SEQ ID NO: 3358.

In another embodiment, the ISVD specifically binding to human PD-1 may have a sequence identity of more than 90%, such as more than 95% or even more than 99%, with SEQ ID NO: 3359, wherein the CDRs are as defined above. In one embodiment, the ISVD specifically binding to PD-1 comprises or consists of the amino acid sequence of SEQ ID NO: 3359.

In another embodiment, the ISVD specifically binding to human PD-1 may have a sequence identity of more than 90%, such as more than 95% or even more than 99%, with SEQ ID NO: 3360, wherein the CDRs are as defined above. In one embodiment, the ISVD specifically binding to PD-1 comprises or consists of the amino acid sequence of SEQ ID NO: 3360.

In another embodiment, the ISVD specifically binding to human PD-1 may have a sequence identity of more than 90%, such as more than 95% or even more than 99%, with SEQ ID NO: 3361, wherein the CDRs are as defined above. In one embodiment, the ISVD specifically binding to PD-1 comprises or consists of the amino acid sequence of SEQ ID NO: 3361.

In another embodiment, the ISVD specifically binding to human PD-1 may have a sequence identity of more than 90%, such as more than 95% or even more than 99%, with SEQ ID NO: 3362, wherein the CDRs are as defined above. In one embodiment, the ISVD specifically binding to PD-1 comprises or consists of the amino acid sequence of SEQ ID NO: 3362.

In another embodiment, the ISVD specifically binding to human PD-1 may have a sequence identity of more than 90%, such as more than 95% or even more than 99%, with SEQ ID NO: 3363, wherein the CDRs are as defined above. In one embodiment, the ISVD specifically binding to PD-1 comprises or consists of the amino acid sequence of SEQ ID NO: 3363.

In another embodiment, the ISVD specifically binding to human PD-1 may have a sequence identity of more than 90%, such as more than 95% or even more than 99%, with SEQ ID NO: 3364, wherein the CDRs are as defined above. In one embodiment, the ISVD specifically binding to PD-1 comprises or consists of the amino acid sequence of SEQ ID NO: 3364.

In another embodiment, the ISVD specifically binding to human PD-1 may have a sequence identity of more than 90%, such as more than 95% or even more than 99%, with SEQ ID NO: 3365, wherein the CDRs are as defined above. In one embodiment, the ISVD specifically binding to PD-1 comprises or consists of the amino acid sequence of SEQ ID NO: 3365.

In another embodiment, the ISVD specifically binding to human PD-1 may have a sequence identity of more than 90%, such as more than 95% or even more than 99%, with SEQ ID NO: 3366, wherein the CDRs are as defined above. In one embodiment, the ISVD specifically binding to PD-1 comprises or consists of the amino acid sequence of SEQ ID NO: 3366.

In another embodiment, the ISVD specifically binding to human PD-1 may have a sequence identity of more than 90%, such as more than 95% or even more than 99%, with SEQ ID NO: 3367, wherein the CDRs are as defined above. In one embodiment, the ISVD specifically binding to PD-1 comprises or consists of the amino acid sequence of SEQ ID NO: 3367.

In another embodiment, the ISVD specifically binding to human PD-1 may have a sequence identity of more than 90%, such as more than 95% or even more than 99%, with SEQ ID NO: 3368, wherein the CDRs are as defined above. In one embodiment, the ISVD specifically binding to PD-1 comprises or consists of the amino acid sequence of SEQ ID NO: 3368.

In another embodiment, the ISVD specifically binding to human PD-1 may have a sequence identity of more than 90%, such as more than 95% or even more than 99%, with SEQ ID NO: 3369, wherein the CDRs are as defined above. In one embodiment, the ISVD specifically binding to PD-1 comprises or consists of the amino acid sequence of SEQ ID NO: 3369.

In another embodiment, the ISVD specifically binding to human PD-1 may have a sequence identity of more than 90%, such as more than 95% or even more than 99%, with SEQ ID NO: 3370, wherein the CDRs are as defined above. In one embodiment, the ISVD specifically binding to PD-1 comprises or consists of the amino acid sequence of SEQ ID NO: 3370.

In another embodiment, the ISVD specifically binding to human PD-1 may have a sequence identity of more than 90%, such as more than 95% or even more than 99%, with SEQ ID NO: 3371, wherein the CDRs are as defined above. In one embodiment, the ISVD specifically binding to PD-1 comprises or consists of the amino acid sequence of SEQ ID NO: 3371.

In another embodiment, the ISVD specifically binding to human PD-1 may have a sequence identity of more than 90%, such as more than 95% or even more than 99%, with SEQ ID NO: 3372, wherein the CDRs are as defined above. In one embodiment, the ISVD specifically binding to PD-1 comprises or consists of the amino acid sequence of SEQ ID NO: 3372.

In another embodiment, the ISVD specifically binding to human PD-1 may have a sequence identity of more than 90%, such as more than 95% or even more than 99%, with SEQ ID NO: 3373, wherein the CDRs are as defined above. In one embodiment, the ISVD specifically binding to PD-1 comprises or consists of the amino acid sequence of SEQ ID NO: 3373.

In another embodiment, the ISVD specifically binding to human PD-1 may have a sequence identity of more than 90%, such as more than 95% or even more than 99%, with SEQ ID NO: 3374, wherein the CDRs are as defined above. In one embodiment, the ISVD specifically binding to PD-1 comprises or consists of the amino acid sequence of SEQ ID NO: 3374.

In another embodiment, the ISVD specifically binding to human PD-1 may have a sequence identity of more than 90%, such as more than 95% or even more than 99%, with SEQ ID NO: 3375, wherein the CDRs are as defined above. In one embodiment, the ISVD specifically binding to PD-1 comprises or consists of the amino acid sequence of SEQ ID NO: 3375.

In another embodiment, the ISVD specifically binding to human PD-1 may have a sequence identity of more than 90%, such as more than 95% or even more than 99%, with SEQ ID NO: 3376, wherein the CDRs are as defined above. In one embodiment, the ISVD specifically binding to PD-1 comprises or consists of the amino acid sequence of SEQ ID NO: 3376.

In another embodiment, the ISVD specifically binding to human PD-1 may have a sequence identity of more than 90%, such as more than 95% or even more than 99%, with SEQ ID NO: 3377, wherein the CDRs are as defined above. In one embodiment, the ISVD specifically binding to PD-1 comprises or consists of the amino acid sequence of SEQ ID NO: 3377.

In another embodiment, the ISVD specifically binding to human PD-1 may have a sequence identity of more than 90%, such as more than 95% or even more than 99%, with SEQ ID NO: 3378, wherein the CDRs are as defined above. In one embodiment, the ISVD specifically binding to PD-1 comprises or consists of the amino acid sequence of SEQ ID NO: 3378.

In another embodiment, the ISVD specifically binding to human PD-1 may have a sequence identity of more than 90%, such as more than 95% or even more than 99%, with SEQ ID NO: 3379, wherein the CDRs are as defined above. In one embodiment, the ISVD specifically binding to PD-1 comprises or consists of the amino acid sequence of SEQ ID NO: 3379.

In another embodiment, the ISVD specifically binding to human PD-1 may have a sequence identity of more than 90%, such as more than 95% or even more than 99%, with SEQ ID NO: 3380, wherein the CDRs are as defined above. In one embodiment, the ISVD specifically binding to PD-1 comprises or consists of the amino acid sequence of SEQ ID NO: 3380.

In another embodiment, the ISVD specifically binding to human PD-1 may have a sequence identity of more than 90%, such as more than 95% or even more than 99%, with SEQ ID NO: 3381, wherein the CDRs are as defined above. In one embodiment, the ISVD specifically binding to PD-1 comprises or consists of the amino acid sequence of SEQ ID NO: 3381.

In another embodiment, the ISVD specifically binding to human PD-1 may have a sequence identity of more than 90%, such as more than 95% or even more than 99%, with SEQ ID NO: 3382, wherein the CDRs are as defined above. In one embodiment, the ISVD specifically binding to PD-1 comprises or consists of the amino acid sequence of SEQ ID NO: 3382.

In another embodiment, the ISVD specifically binding to human PD-1 may have a sequence identity of more than 90%, such as more than 95% or even more than 99%, with SEQ ID NO: 3383, wherein the CDRs are as defined above. In one embodiment, the ISVD specifically binding to PD-1 comprises or consists of the amino acid sequence of SEQ ID NO: 3383.

In another embodiment, the ISVD specifically binding to human PD-1 may have a sequence identity of more than 90%, such as more than 95% or even more than 99%, with SEQ ID NO: 3384, wherein the CDRs are as defined above. In one embodiment, the ISVD specifically binding to PD-1 comprises or consists of the amino acid sequence of SEQ ID NO: 3384.

In another embodiment, the ISVD specifically binding to human PD-1 may have a sequence identity of more than 90%, such as more than 95% or even more than 99%, with SEQ ID NO: 3385, wherein the CDRs are as defined above. In one embodiment, the ISVD specifically binding to PD-1 comprises or consists of the amino acid sequence of SEQ ID NO: 3385.

In another embodiment, the ISVD specifically binding to human PD-1 may have a sequence identity of more than 90%, such as more than 95% or even more than 99%, with SEQ ID NO: 3386, wherein the CDRs are as defined above. In one embodiment, the ISVD specifically binding to PD-1 comprises or consists of the amino acid sequence of SEQ ID NO: 3386.

In another embodiment, the ISVD specifically binding to human PD-1 may have a sequence identity of more than 90%, such as more than 95% or even more than 99%, with SEQ ID NO: 3387, wherein the CDRs are as defined above. In one embodiment, the ISVD specifically binding to PD-1 comprises or consists of the amino acid sequence of SEQ ID NO: 3387.

In another embodiment, the ISVD specifically binding to human PD-1 may have a sequence identity of more than 90%, such as more than 95% or even more than 99%, with SEQ ID NO: 3388, wherein the CDRs are as defined above. In one embodiment, the ISVD specifically binding to PD-1 comprises or consists of the amino acid sequence of SEQ ID NO: 3388.

In another embodiment, the ISVD specifically binding to human PD-1 may have a sequence identity of more than 90%, such as more than 95% or even more than 99%, with SEQ ID NO: 3389, wherein the CDRs are as defined above. In one embodiment, the ISVD specifically binding to PD-1 comprises or consists of the amino acid sequence of SEQ ID NO: 3389.

In another embodiment, the ISVD specifically binding to human PD-1 may have a sequence identity of more than 90%, such as more than 95% or even more than 99%, with SEQ ID NO: 3390, wherein the CDRs are as defined above. In one embodiment, the ISVD specifically binding to PD-1 comprises or consists of the amino acid sequence of SEQ ID NO: 3390.

In another embodiment, the ISVD specifically binding to human PD-1 may have a sequence identity of more than 90%, such as more than 95% or even more than 99%, with SEQ ID NO: 3391, wherein the CDRs are as defined above. In one embodiment, the ISVD specifically binding to PD-1 comprises or consists of the amino acid sequence of SEQ ID NO: 3391.

In another embodiment, the ISVD specifically binding to human PD-1 may have a sequence identity of more than 90%, such as more than 95% or even more than 99%, with SEQ ID NO: 3392, wherein the CDRs are as defined above. In one embodiment, the ISVD specifically binding to PD-1 comprises or consists of the amino acid sequence of SEQ ID NO: 3392.

In another embodiment, the ISVD specifically binding to human PD-1 may have a sequence identity of more than 90%, such as more than 95% or even more than 99%, with SEQ ID NO: 3393, wherein the CDRs are as defined above. In one embodiment, the ISVD specifically binding to PD-1 comprises or consists of the amino acid sequence of SEQ ID NO: 3393.

In another embodiment, the ISVD specifically binding to human PD-1 may have a sequence identity of more than 90%, such as more than 95% or even more than 99%, with SEQ ID NO: 3394, wherein the CDRs are as defined above. In one embodiment, the ISVD specifically binding to PD-1 comprises or consists of the amino acid sequence of SEQ ID NO: 3394.

In another embodiment, the ISVD specifically binding to human PD-1 may have a sequence identity of more than 90%, such as more than 95% or even more than 99%, with SEQ ID NO: 3395, wherein the CDRs are as defined above. In one embodiment, the ISVD specifically binding to PD-1 comprises or consists of the amino acid sequence of SEQ ID NO: 3395.

In another embodiment, the ISVD specifically binding to human PD-1 may have a sequence identity of more than 90%, such as more than 95% or even more than 99%, with SEQ ID NO: 3396, wherein the CDRs are as defined above. In one embodiment, the ISVD specifically binding to PD-1 comprises or consists of the amino acid sequence of SEQ ID NO: 3396.

In another embodiment, the ISVD specifically binding to human PD-1 may have a sequence identity of more than 90%, such as more than 95% or even more than 99%, with SEQ ID NO: 3397, wherein the CDRs are as defined above. In one embodiment, the ISVD specifically binding to PD-1 comprises or consists of the amino acid sequence of SEQ ID NO: 3397.

In another embodiment, the ISVD specifically binding to human PD-1 may have a sequence identity of more than 90%, such as more than 95% or even more than 99%, with SEQ ID NO: 3398, wherein the CDRs are as defined above. In one embodiment, the ISVD specifically binding to PD-1 comprises or consists of the amino acid sequence of SEQ ID NO: 3398.

In another embodiment, the ISVD specifically binding to human PD-1 may have a sequence identity of more than 90%, such as more than 95% or even more than 99%, with SEQ ID NO: 3399, wherein the CDRs are as defined above. In one embodiment, the ISVD specifically binding to PD-1 comprises or consists of the amino acid sequence of SEQ ID NO: 3399.

In another embodiment, the ISVD specifically binding to human PD-1 may have a sequence identity of more than 90%, such as more than 95% or even more than 99%, with SEQ ID NO: 3400, wherein the CDRs are as defined above. In one embodiment, the ISVD specifically binding to PD-1 comprises or consists of the amino acid sequence of SEQ ID NO: 3400.

In another embodiment, the ISVD specifically binding to human PD-1 may have a sequence identity of more than 90%, such as more than 95% or even more than 99%, with SEQ ID NO: 3401, wherein the CDRs are as defined above. In one embodiment, the ISVD specifically binding to PD-1 comprises or consists of the amino acid sequence of SEQ ID NO: 3401.

In another embodiment, the ISVD specifically binding to human PD-1 may have a sequence identity of more than 90%, such as more than 95% or even more than 99%, with SEQ ID NO: 3402, wherein the CDRs are as defined above. In one embodiment, the ISVD specifically binding to PD-1 comprises or consists of the amino acid sequence of SEQ ID NO: 3402.

In another embodiment, the ISVD specifically binding to human PD-1 may have a sequence identity of more than 90%, such as more than 95% or even more than 99%, with SEQ ID NO: 3403, wherein the CDRs are as defined above. In one embodiment, the ISVD specifically binding to PD-1 comprises or consists of the amino acid sequence of SEQ ID NO: 3403.

In another embodiment, the ISVD specifically binding to human PD-1 may have a sequence identity of more than 90%, such as more than 95% or even more than 99%, with SEQ ID NO: 3404, wherein the CDRs are as defined above. In one embodiment, the ISVD specifically binding to PD-1 comprises or consists of the amino acid sequence of SEQ ID NO: 3404.

In another embodiment, the ISVD specifically binding to human PD-1 may have a sequence identity of more than 90%, such as more than 95% or even more than 99%, with SEQ ID NO: 3405, wherein the CDRs are as defined above. In one embodiment, the ISVD specifically binding to PD-1 comprises or consists of the amino acid sequence of SEQ ID NO: 3405.

In another embodiment, the ISVD specifically binding to human PD-1 may have a sequence identity of more than 90%, such as more than 95% or even more than 99%, with SEQ ID NO: 3406, wherein the CDRs are as defined above. In one embodiment, the ISVD specifically binding to PD-1 comprises or consists of the amino acid sequence of SEQ ID NO: 3406.

In another embodiment, the ISVD specifically binding to human PD-1 may have a sequence identity of more than 90%, such as more than 95% or even more than 99%, with SEQ ID NO: 3407, wherein the CDRs are as defined above. In one embodiment, the ISVD specifically binding to PD-1 comprises or consists of the amino acid sequence of SEQ ID NO: 3407.

In another embodiment, the ISVD specifically binding to human PD-1 may have a sequence identity of more than 90%, such as more than 95% or even more than 99%, with SEQ ID NO: 3408, wherein the CDRs are as defined above. In one embodiment, the ISVD specifically binding to PD-1 comprises or consists of the amino acid sequence of SEQ ID NO: 3408.

In another embodiment, the ISVD specifically binding to human PD-1 may have a sequence identity of more than 90%, such as more than 95% or even more than 99%, with SEQ ID NO: 3409, wherein the CDRs are as defined above. In one embodiment, the ISVD specifically binding to PD-1 comprises or consists of the amino acid sequence of SEQ ID NO: 3409.

In another embodiment, the ISVD specifically binding to human PD-1 may have a sequence identity of more than 90%, such as more than 95% or even more than 99%, with SEQ ID NO: 3410, wherein the CDRs are as defined above. In one embodiment, the ISVD specifically binding to PD-1 comprises or consists of the amino acid sequence of SEQ ID NO: 3410.

In another embodiment, the ISVD specifically binding to human PD-1 may have a sequence identity of more than 90%, such as more than 95% or even more than 99%, with SEQ ID NO: 3411, wherein the CDRs are as defined above. In one embodiment, the ISVD specifically binding to PD-1 comprises or consists of the amino acid sequence of SEQ ID NO: 3411.

In another embodiment, the ISVD specifically binding to human PD-1 may have a sequence identity of more than 90%, such as more than 95% or even more than 99%, with SEQ ID NO: 3412, wherein the CDRs are as defined above. In one embodiment, the ISVD specifically binding to PD-1 comprises or consists of the amino acid sequence of SEQ ID NO: 3412.

In another embodiment, the ISVD specifically binding to human PD-1 may have a sequence identity of more than 90%, such as more than 95% or even more than 99%, with SEQ ID NO: 3413, wherein the CDRs are as defined above. In one embodiment, the ISVD specifically binding to PD-1 comprises or consists of the amino acid sequence of SEQ ID NO: 3413.

In another embodiment, the ISVD specifically binding to human PD-1 may have a sequence identity of more than 90%, such as more than 95% or even more than 99%, with SEQ ID NO: 3414, wherein the CDRs are as defined above. In one embodiment, the ISVD specifically binding to PD-1 comprises or consists of the amino acid sequence of SEQ ID NO: 3414.

In another embodiment, the ISVD specifically binding to human PD-1 may have a sequence identity of more than 90%, such as more than 95% or even more than 99%, with SEQ ID NO: 3415, wherein the CDRs are as defined above. In one embodiment, the ISVD specifically binding to PD-1 comprises or consists of the amino acid sequence of SEQ ID NO: 3415.

In another embodiment, the ISVD specifically binding to human PD-1 may have a sequence identity of more than 90%, such as more than 95% or even more than 99%, with SEQ ID NO: 3416, wherein the CDRs are as defined above. In one embodiment, the ISVD specifically binding to PD-1 comprises or consists of the amino acid sequence of SEQ ID NO: 3416.

In another embodiment, the ISVD specifically binding to human PD-1 may have a sequence identity of more than 90%, such as more than 95% or even more than 99%, with SEQ ID NO: 3417, wherein the CDRs are as defined above. In one embodiment, the ISVD specifically binding to PD-1 comprises or consists of the amino acid sequence of SEQ ID NO: 3417.

In another embodiment, the ISVD specifically binding to human PD-1 may have a sequence identity of more than 90%, such as more than 95% or even more than 99%, with SEQ ID NO: 3418, wherein the CDRs are as defined above. In one embodiment, the ISVD specifically binding to PD-1 comprises or consists of the amino acid sequence of SEQ ID NO: 3418.

In another embodiment, the ISVD specifically binding to human PD-1 may have a sequence identity of more than 90%, such as more than 95% or even more than 99%, with SEQ ID NO: 3419, wherein the CDRs are as defined above. In one embodiment, the ISVD specifically binding to PD-1 comprises or consists of the amino acid sequence of SEQ ID NO: 3419.

In another embodiment, the ISVD specifically binding to human PD-1 may have a sequence identity of more than 90%, such as more than 95% or even more than 99%, with SEQ ID NO: 3420, wherein the CDRs are as defined above. In one embodiment, the ISVD specifically binding to PD-1 comprises or consists of the amino acid sequence of SEQ ID NO: 3420.

In another embodiment, the ISVD specifically binding to human PD-1 may have a sequence identity of more than 90%, such as more than 95% or even more than 99%, with SEQ ID NO: 3421, wherein the CDRs are as defined above. In one embodiment, the ISVD specifically binding to PD-1 comprises or consists of the amino acid sequence of SEQ ID NO: 3421.

In another embodiment, the ISVD specifically binding to human PD-1 may have a sequence identity of more than 90%, such as more than 95% or even more than 99%, with SEQ ID NO: 3422, wherein the CDRs are as defined above. In one embodiment, the ISVD specifically binding to PD-1 comprises or consists of the amino acid sequence of SEQ ID NO: 3422.

In another embodiment, the ISVD specifically binding to human PD-1 may have a sequence identity of more than 90%, such as more than 95% or even more than 99%, with SEQ ID NO: 3423, wherein the CDRs are as defined above. In one embodiment, the ISVD specifically binding to PD-1 comprises or consists of the amino acid sequence of SEQ ID NO: 3423.

In another embodiment, the ISVD specifically binding to human PD-1 may have a sequence identity of more than 90%, such as more than 95% or even more than 99%, with SEQ ID NO: 3424, wherein the CDRs are as defined above. In one embodiment, the ISVD specifically binding to PD-1 comprises or consists of the amino acid sequence of SEQ ID NO: 3424.

In another embodiment, the ISVD specifically binding to human PD-1 may have a sequence identity of more than 90%, such as more than 95% or even more than 99%, with SEQ ID NO: 3425, wherein the CDRs are as defined above. In one embodiment, the ISVD specifically binding to PD-1 comprises or consists of the amino acid sequence of SEQ ID NO: 3425.

In another embodiment, the ISVD specifically binding to human PD-1 may have a sequence identity of more than 90%, such as more than 95% or even more than 99%, with SEQ ID NO: 3426, wherein the CDRs are as defined above. In one embodiment, the ISVD specifically binding to PD-1 comprises or consists of the amino acid sequence of SEQ ID NO: 3426.

In another embodiment, the ISVD specifically binding to human PD-1 may have a sequence identity of more than 90%, such as more than 95% or even more than 99%, with SEQ ID NO: 3427, wherein the CDRs are as defined above. In one embodiment, the ISVD specifically binding to PD-1 comprises or consists of the amino acid sequence of SEQ ID NO: 3427.

In another embodiment, the ISVD specifically binding to human PD-1 may have a sequence identity of more than 90%, such as more than 95% or even more than 99%, with SEQ ID NO: 3428, wherein the CDRs are as defined above. In one embodiment, the ISVD specifically binding to PD-1 comprises or consists of the amino acid sequence of SEQ ID NO: 3428.

In another embodiment, the ISVD specifically binding to human PD-1 may have a sequence identity of more than 90%, such as more than 95% or even more than 99%, with SEQ ID NO: 3429, wherein the CDRs are as defined above. In one embodiment, the ISVD specifically binding to PD-1 comprises or consists of the amino acid sequence of SEQ ID NO: 3429.

In another embodiment, the ISVD specifically binding to human PD-1 may have a sequence identity of more than 90%, such as more than 95% or even more than 99%, with SEQ ID NO: 3430, wherein the CDRs are as defined above. In one embodiment, the ISVD specifically binding to PD-1 comprises or consists of the amino acid sequence of SEQ ID NO: 3430.

In another embodiment, the ISVD specifically binding to human PD-1 may have a sequence identity of more than 90%, such as more than 95% or even more than 99%, with SEQ ID NO: 3431, wherein the CDRs are as defined above. In one embodiment, the ISVD specifically binding to PD-1 comprises or consists of the amino acid sequence of SEQ ID NO: 3431.

In another embodiment, the ISVD specifically binding to human PD-1 may have a sequence identity of more than 90%, such as more than 95% or even more than 99%, with SEQ ID NO: 3432, wherein the CDRs are as defined above. In one embodiment, the ISVD specifically binding to PD-1 comprises or consists of the amino acid sequence of SEQ ID NO: 3432.

In another embodiment, the ISVD specifically binding to human PD-1 may have a sequence identity of more than 90%, such as more than 95% or even more than 99%, with SEQ ID NO: 3433, wherein the CDRs are as defined above. In one embodiment, the ISVD specifically binding to PD-1 comprises or consists of the amino acid sequence of SEQ ID NO: 3433.

In another embodiment, the ISVD specifically binding to human PD-1 may have a sequence identity of more than 90%, such as more than 95% or even more than 99%, with SEQ ID NO: 3434, wherein the CDRs are as defined above. In one embodiment, the ISVD specifically binding to PD-1 comprises or consists of the amino acid sequence of SEQ ID NO: 3434.

In another embodiment, the ISVD specifically binding to human PD-1 may have a sequence identity of more than 90%, such as more than 95% or even more than 99%, with SEQ ID NO: 3435, wherein the CDRs are as defined above. In one embodiment, the ISVD specifically binding to PD-1 comprises or consists of the amino acid sequence of SEQ ID NO: 3435.

In another embodiment, the ISVD specifically binding to human PD-1 may have a sequence identity of more than 90%, such as more than 95% or even more than 99%, with SEQ ID NO: 3436, wherein the CDRs are as defined above. In one embodiment, the ISVD specifically binding to PD-1 comprises or consists of the amino acid sequence of SEQ ID NO: 3436.

In another embodiment, the ISVD specifically binding to human PD-1 may have a sequence identity of more than 90%, such as more than 95% or even more than 99%, with SEQ ID NO: 3437, wherein the CDRs are as defined above. In one embodiment, the ISVD specifically binding to PD-1 comprises or consists of the amino acid sequence of SEQ ID NO: 3437.

In another embodiment, the ISVD specifically binding to human PD-1 may have a sequence identity of more than 90%, such as more than 95% or even more than 99%, with SEQ ID NO: 3438, wherein the CDRs are as defined above. In one embodiment, the ISVD specifically binding to PD-1 comprises or consists of the amino acid sequence of SEQ ID NO: 3438.

In another embodiment, the ISVD specifically binding to human PD-1 may have a sequence identity of more than 90%, such as more than 95% or even more than 99%, with SEQ ID NO: 3439, wherein the CDRs are as defined above. In one embodiment, the ISVD specifically binding to PD-1 comprises or consists of the amino acid sequence of SEQ ID NO: 3439.

In another embodiment, the ISVD specifically binding to human PD-1 may have a sequence identity of more than 90%, such as more than 95% or even more than 99%, with SEQ ID NO: 3440, wherein the CDRs are as defined above. In one embodiment, the ISVD specifically binding to PD-1 comprises or consists of the amino acid sequence of SEQ ID NO: 3440.

In another embodiment, the ISVD specifically binding to human PD-1 may have a sequence identity of more than 90%, such as more than 95% or even more than 99%, with SEQ ID NO: 3441, wherein the CDRs are as defined above. In one embodiment, the ISVD specifically binding to PD-1 comprises or consists of the amino acid sequence of SEQ ID NO: 3441.

In another embodiment, the ISVD specifically binding to human PD-1 may have a sequence identity of more than 90%, such as more than 95% or even more than 99%, with SEQ ID NO: 3442, wherein the CDRs are as defined above. In one embodiment, the ISVD specifically binding to PD-1 comprises or consists of the amino acid sequence of SEQ ID NO: 3442.

In another embodiment, the ISVD specifically binding to human PD-1 may have a sequence identity of more than 90%, such as more than 95% or even more than 99%, with SEQ ID NO: 3443, wherein the CDRs are as defined above. In one embodiment, the ISVD specifically binding to PD-1 comprises or consists of the amino acid sequence of SEQ ID NO: 3443.

In another embodiment, the ISVD specifically binding to human PD-1 may have a sequence identity of more than 90%, such as more than 95% or even more than 99%, with SEQ ID NO: 3444, wherein the CDRs are as defined above. In one embodiment, the ISVD specifically binding to PD-1 comprises or consists of the amino acid sequence of SEQ ID NO: 3444.

In another embodiment, the ISVD specifically binding to human PD-1 may have a sequence identity of more than 90%, such as more than 95% or even more than 99%, with SEQ ID NO: 3445, wherein the CDRs are as defined above. In one embodiment, the ISVD specifically binding to PD-1 comprises or consists of the amino acid sequence of SEQ ID NO: 3445.

In another embodiment, the ISVD specifically binding to human PD-1 may have a sequence identity of more than 90%, such as more than 95% or even more than 99%, with SEQ ID NO: 3446, wherein the CDRs are as defined above. In one embodiment, the ISVD specifically binding to PD-1 comprises or consists of the amino acid sequence of SEQ ID NO: 3446.

In another embodiment, the ISVD specifically binding to human PD-1 may have a sequence identity of more than 90%, such as more than 95% or even more than 99%, with SEQ ID NO: 3447, wherein the CDRs are as defined above. In one embodiment, the ISVD specifically binding to PD-1 comprises or consists of the amino acid sequence of SEQ ID NO: 3447.

In another embodiment, the ISVD specifically binding to human PD-1 may have a sequence identity of more than 90%, such as more than 95% or even more than 99%, with SEQ ID NO: 3448, wherein the CDRs are as defined above. In one embodiment, the ISVD specifically binding to PD-1 comprises or consists of the amino acid sequence of SEQ ID NO: 3448.

In another embodiment, the ISVD specifically binding to human PD-1 may have a sequence identity of more than 90%, such as more than 95% or even more than 99%, with SEQ ID NO: 3449, wherein the CDRs are as defined above. In one embodiment, the ISVD specifically binding to PD-1 comprises or consists of the amino acid sequence of SEQ ID NO: 3449.

In another embodiment, the ISVD specifically binding to human PD-1 may have a sequence identity of more than 90%, such as more than 95% or even more than 99%, with SEQ ID NO: 3450, wherein the CDRs are as defined above. In one embodiment, the ISVD specifically binding to PD-1 comprises or consists of the amino acid sequence of SEQ ID NO: 3450.

In another embodiment, the ISVD specifically binding to human PD-1 may have a sequence identity of more than 90%, such as more than 95% or even more than 99%, with SEQ ID NO: 3451, wherein the CDRs are as defined above. In one embodiment, the ISVD specifically binding to PD-1 comprises or consists of the amino acid sequence of SEQ ID NO: 3451.

In another embodiment, the ISVD specifically binding to human PD-1 may have a sequence identity of more than 90%, such as more than 95% or even more than 99%, with SEQ ID NO: 3452, wherein the CDRs are as defined above. In one embodiment, the ISVD specifically binding to PD-1 comprises or consists of the amino acid sequence of SEQ ID NO: 3452.

In another embodiment, the ISVD specifically binding to human PD-1 may have a sequence identity of more than 90%, such as more than 95% or even more than 99%, with SEQ ID NO: 3453, wherein the CDRs are as defined above. In one embodiment, the ISVD specifically binding to PD-1 comprises or consists of the amino acid sequence of SEQ ID NO: 3453.

In another embodiment, the ISVD specifically binding to human PD-1 may have a sequence identity of more than 90%, such as more than 95% or even more than 99%, with SEQ ID NO: 3454, wherein the CDRs are as defined above. In one embodiment, the ISVD specifically binding to PD-1 comprises or consists of the amino acid sequence of SEQ ID NO: 3454.

In another embodiment, the ISVD specifically binding to human PD-1 may have a sequence identity of more than 90%, such as more than 95% or even more than 99%, with SEQ ID NO: 3455, wherein the CDRs are as defined above. In one embodiment, the ISVD specifically binding to PD-1 comprises or consists of the amino acid sequence of SEQ ID NO: 3455.

In another embodiment, the ISVD specifically binding to human PD-1 may have a sequence identity of more than 90%, such as more than 95% or even more than 99%, with SEQ ID NO: 3456, wherein the CDRs are as defined above. In one embodiment, the ISVD specifically binding to PD-1 comprises or consists of the amino acid sequence of SEQ ID NO: 3456.

In another embodiment, the ISVD specifically binding to human PD-1 may have a sequence identity of more than 90%, such as more than 95% or even more than 99%, with SEQ ID NO: 3457, wherein the CDRs are as defined above. In one embodiment, the ISVD specifically binding to PD-1 comprises or consists of the amino acid sequence of SEQ ID NO: 3457.

In another embodiment, the ISVD specifically binding to human PD-1 may have a sequence identity of more than 90%, such as more than 95% or even more than 99%, with SEQ ID NO: 3458, wherein the CDRs are as defined above. In one embodiment, the ISVD specifically binding to PD-1 comprises or consists of the amino acid sequence of SEQ ID NO: 3458.

In another embodiment, the ISVD specifically binding to human PD-1 may have a sequence identity of more than 90%, such as more than 95% or even more than 99%, with SEQ ID NO: 3459, wherein the CDRs are as defined above. In one embodiment, the ISVD specifically binding to PD-1 comprises or consists of the amino acid sequence of SEQ ID NO: 3459.

In another embodiment, the ISVD specifically binding to human PD-1 may have a sequence identity of more than 90%, such as more than 95% or even more than 99%, with SEQ ID NO: 3460, wherein the CDRs are as defined above. In one embodiment, the ISVD specifically binding to PD-1 comprises or consists of the amino acid sequence of SEQ ID NO: 3460.

In another embodiment, the ISVD specifically binding to human PD-1 may have a sequence identity of more than 90%, such as more than 95% or even more than 99%, with SEQ ID NO: 3461, wherein the CDRs are as defined above. In one embodiment, the ISVD specifically binding to PD-1 comprises or consists of the amino acid sequence of SEQ ID NO: 3461.

In another embodiment, the ISVD specifically binding to human PD-1 may have a sequence identity of more than 90%, such as more than 95% or even more than 99%, with SEQ ID NO: 3462, wherein the CDRs are as defined above. In one embodiment, the ISVD specifically binding to PD-1 comprises or consists of the amino acid sequence of SEQ ID NO: 3462.

In another embodiment, the ISVD specifically binding to human PD-1 may have a sequence identity of more than 90%, such as more than 95% or even more than 99%, with SEQ ID NO: 3463, wherein the CDRs are as defined above. In one embodiment, the ISVD specifically binding to PD-1 comprises or consists of the amino acid sequence of SEQ ID NO: 3463.

In another embodiment, the ISVD specifically binding to human PD-1 may have a sequence identity of more than 90%, such as more than 95% or even more than 99%, with SEQ ID NO: 3464, wherein the CDRs are as defined above. In one embodiment, the ISVD specifically binding to PD-1 comprises or consists of the amino acid sequence of SEQ ID NO: 3464.

In another embodiment, the ISVD specifically binding to human PD-1 may have a sequence identity of more than 90%, such as more than 95% or even more than 99%, with SEQ ID NO: 3465, wherein the CDRs are as defined above. In one embodiment, the ISVD specifically binding to PD-1 comprises or consists of the amino acid sequence of SEQ ID NO: 3465.

In another embodiment, the ISVD specifically binding to human PD-1 may have a sequence identity of more than 90%, such as more than 95% or even more than 99%, with SEQ ID NO: 3466, wherein the CDRs are as defined above. In one embodiment, the ISVD specifically binding to PD-1 comprises or consists of the amino acid sequence of SEQ ID NO: 3466.

In another embodiment, the ISVD specifically binding to human PD-1 may have a sequence identity of more than 90%, such as more than 95% or even more than 99%, with SEQ ID NO: 3467, wherein the CDRs are as defined above. In one embodiment, the ISVD specifically binding to PD-1 comprises or consists of the amino acid sequence of SEQ ID NO: 3467.

In another embodiment, the ISVD specifically binding to human PD-1 may have a sequence identity of more than 90%, such as more than 95% or even more than 99%, with SEQ ID NO: 3468, wherein the CDRs are as defined above. In one embodiment, the ISVD specifically binding to PD-1 comprises or consists of the amino acid sequence of SEQ ID NO: 3468.

In another embodiment, the ISVD specifically binding to human PD-1 may have a sequence identity of more than 90%, such as more than 95% or even more than 99%, with SEQ ID NO: 3469, wherein the CDRs are as defined above. In one embodiment, the ISVD specifically binding to PD-1 comprises or consists of the amino acid sequence of SEQ ID NO: 3469.

In another embodiment, the ISVD specifically binding to human PD-1 may have a sequence identity of more than 90%, such as more than 95% or even more than 99%, with SEQ ID NO: 3470, wherein the CDRs are as defined above. In one embodiment, the ISVD specifically binding to PD-1 comprises or consists of the amino acid sequence of SEQ ID NO: 3470.

In another embodiment, the ISVD specifically binding to human PD-1 may have a sequence identity of more than 90%, such as more than 95% or even more than 99%, with SEQ ID NO: 3471, wherein the CDRs are as defined above. In one embodiment, the ISVD specifically binding to PD-1 comprises or consists of the amino acid sequence of SEQ ID NO: 3471.

In another embodiment, the ISVD specifically binding to human PD-1 may have a sequence identity of more than 90%, such as more than 95% or even more than 99%, with SEQ ID NO: 3472, wherein the CDRs are as defined above. In one embodiment, the ISVD specifically binding to PD-1 comprises or consists of the amino acid sequence of SEQ ID NO: 3472.

In another embodiment, the ISVD specifically binding to human PD-1 may have a sequence identity of more than 90%, such as more than 95% or even more than 99%, with SEQ ID NO: 3473, wherein the CDRs are as defined above. In one embodiment, the ISVD specifically binding to PD-1 comprises or consists of the amino acid sequence of SEQ ID NO: 3473.

In another embodiment, the ISVD specifically binding to human PD-1 may have a sequence identity of more than 90%, such as more than 95% or even more than 99%, with SEQ ID NO: 3474, wherein the CDRs are as defined above. In one embodiment, the ISVD specifically binding to PD-1 comprises or consists of the amino acid sequence of SEQ ID NO: 3474.

In another embodiment, the ISVD specifically binding to human PD-1 may have a sequence identity of more than 90%, such as more than 95% or even more than 99%, with SEQ ID NO: 3475, wherein the CDRs are as defined above. In one embodiment, the ISVD specifically binding to PD-1 comprises or consists of the amino acid sequence of SEQ ID NO: 3475.

In another embodiment, the ISVD specifically binding to human PD-1 may have a sequence identity of more than 90%, such as more than 95% or even more than 99%, with SEQ ID NO: 3476, wherein the CDRs are as defined above. In one embodiment, the ISVD specifically binding to PD-1 comprises or consists of the amino acid sequence of SEQ ID NO: 3476.

In another embodiment, the ISVD specifically binding to human PD-1 may have a sequence identity of more than 90%, such as more than 95% or even more than 99%, with SEQ ID NO: 3477, wherein the CDRs are as defined above. In one embodiment, the ISVD specifically binding to PD-1 comprises or consists of the amino acid sequence of SEQ ID NO: 3477.

In another embodiment, the ISVD specifically binding to human PD-1 may have a sequence identity of more than 90%, such as more than 95% or even more than 99%, with SEQ ID NO: 3478, wherein the CDRs are as defined above. In one embodiment, the ISVD specifically binding to PD-1 comprises or consists of the amino acid sequence of SEQ ID NO: 3478.

In another embodiment, the ISVD specifically binding to human PD-1 may have a sequence identity of more than 90%, such as more than 95% or even more than 99%, with SEQ ID NO: 3479, wherein the CDRs are as defined above. In one embodiment, the ISVD specifically binding to PD-1 comprises or consists of the amino acid sequence of SEQ ID NO: 3479.

In another embodiment, the ISVD specifically binding to human PD-1 may have a sequence identity of more than 90%, such as more than 95% or even more than 99%, with SEQ ID NO: 3480, wherein the CDRs are as defined above. In one embodiment, the ISVD specifically binding to PD-1 comprises or consists of the amino acid sequence of SEQ ID NO: 3480.

In another embodiment, the ISVD specifically binding to human PD-1 may have a sequence identity of more than 90%, such as more than 95% or even more than 99%, with SEQ ID NO: 3481, wherein the CDRs are as defined above. In one embodiment, the ISVD specifically binding to PD-1 comprises or consists of the amino acid sequence of SEQ ID NO: 3481.

In another embodiment, the ISVD specifically binding to human PD-1 may have a sequence identity of more than 90%, such as more than 95% or even more than 99%, with SEQ ID NO: 3482, wherein the CDRs are as defined above. In one embodiment, the ISVD specifically binding to PD-1 comprises or consists of the amino acid sequence of SEQ ID NO: 3482.

In another embodiment, the ISVD specifically binding to human PD-1 may have a sequence identity of more than 90%, such as more than 95% or even more than 99%, with SEQ ID NO: 3483, wherein the CDRs are as defined above. In one embodiment, the ISVD specifically binding to PD-1 comprises or consists of the amino acid sequence of SEQ ID NO: 3483.

In another embodiment, the ISVD specifically binding to human PD-1 may have a sequence identity of more than 90%, such as more than 95% or even more than 99%, with SEQ ID NO: 3484, wherein the CDRs are as defined above. In one embodiment, the ISVD specifically binding to PD-1 comprises or consists of the amino acid sequence of SEQ ID NO: 3484.

In another embodiment, the ISVD specifically binding to human PD-1 may have a sequence identity of more than 90%, such as more than 95% or even more than 99%, with SEQ ID NO: 3485, wherein the CDRs are as defined above. In one embodiment, the ISVD specifically binding to PD-1 comprises or consists of the amino acid sequence of SEQ ID NO: 3485.

In another embodiment, the ISVD specifically binding to human PD-1 may have a sequence identity of more than 90%, such as more than 95% or even more than 99%, with SEQ ID NO: 3486, wherein the CDRs are as defined above. In one embodiment, the ISVD specifically binding to PD-1 comprises or consists of the amino acid sequence of SEQ ID NO: 3486.

In another embodiment, the ISVD specifically binding to human PD-1 may have a sequence identity of more than 90%, such as more than 95% or even more than 99%, with SEQ ID NO: 3487, wherein the CDRs are as defined above. In one embodiment, the ISVD specifically binding to PD-1 comprises or consists of the amino acid sequence of SEQ ID NO: 3487.

In another embodiment, the ISVD specifically binding to human PD-1 may have a sequence identity of more than 90%, such as more than 95% or even more than 99%, with SEQ ID NO: 3488, wherein the CDRs are as defined above. In one embodiment, the ISVD specifically binding to PD-1 comprises or consists of the amino acid sequence of SEQ ID NO: 3488.

In another embodiment, the ISVD specifically binding to human PD-1 may have a sequence identity of more than 90%, such as more than 95% or even more than 99%, with SEQ ID NO: 3489, wherein the CDRs are as defined above. In one embodiment, the ISVD specifically binding to PD-1 comprises or consists of the amino acid sequence of SEQ ID NO: 3489.

In another embodiment, the ISVD specifically binding to human PD-1 may have a sequence identity of more than 90%, such as more than 95% or even more than 99%, with SEQ ID NO: 3490, wherein the CDRs are as defined above. In one embodiment, the ISVD specifically binding to PD-1 comprises or consists of the amino acid sequence of SEQ ID NO: 3490.

In another embodiment, the ISVD specifically binding to human PD-1 may have a sequence identity of more than 90%, such as more than 95% or even more than 99%, with SEQ ID NO: 3491, wherein the CDRs are as defined above. In one embodiment, the ISVD specifically binding to PD-1 comprises or consists of the amino acid sequence of SEQ ID NO: 3491.

In another embodiment, the ISVD specifically binding to human PD-1 may have a sequence identity of more than 90%, such as more than 95% or even more than 99%, with SEQ ID NO: 3492, wherein the CDRs are as defined above. In one embodiment, the ISVD specifically binding to PD-1 comprises or consists of the amino acid sequence of SEQ ID NO: 3492.

In another embodiment, the ISVD specifically binding to human PD-1 may have a sequence identity of more than 90%, such as more than 95% or even more than 99%, with SEQ ID NO: 3493, wherein the CDRs are as defined above. In one embodiment, the ISVD specifically binding to PD-1 comprises or consists of the amino acid sequence of SEQ ID NO: 3493.

In another embodiment, the ISVD specifically binding to human PD-1 may have a sequence identity of more than 90%, such as more than 95% or even more than 99%, with SEQ ID NO: 3494, wherein the CDRs are as defined above. In one embodiment, the ISVD specifically binding to PD-1 comprises or consists of the amino acid sequence of SEQ ID NO: 3494.

In another embodiment, the ISVD specifically binding to human PD-1 may have a sequence identity of more than 90%, such as more than 95% or even more than 99%, with SEQ ID NO: 3495, wherein the CDRs are as defined above. In one embodiment, the ISVD specifically binding to PD-1 comprises or consists of the amino acid sequence of SEQ ID NO: 3495.

In another embodiment, the ISVD specifically binding to human PD-1 may have a sequence identity of more than 90%, such as more than 95% or even more than 99%, with SEQ ID NO: 3496, wherein the CDRs are as defined above. In one embodiment, the ISVD specifically binding to PD-1 comprises or consists of the amino acid sequence of SEQ ID NO: 3496.

In another embodiment, the ISVD specifically binding to human PD-1 may have a sequence identity of more than 90%, such as more than 95% or even more than 99%, with SEQ ID NO: 3497, wherein the CDRs are as defined above. In one embodiment, the ISVD specifically binding to PD-1 comprises or consists of the amino acid sequence of SEQ ID NO: 3497.

In another embodiment, the ISVD specifically binding to human PD-1 may have a sequence identity of more than 90%, such as more than 95% or even more than 99%, with SEQ ID NO: 3498, wherein the CDRs are as defined above. In one embodiment, the ISVD specifically binding to PD-1 comprises or consists of the amino acid sequence of SEQ ID NO: 3498.

In another embodiment, the ISVD specifically binding to human PD-1 may have a sequence identity of more than 90%, such as more than 95% or even more than 99%, with SEQ ID NO: 3499, wherein the CDRs are as defined above. In one embodiment, the ISVD specifically binding to PD-1 comprises or consists of the amino acid sequence of SEQ ID NO: 3499.

In another embodiment, the ISVD specifically binding to human PD-1 may have a sequence identity of more than 90%, such as more than 95% or even more than 99%, with SEQ ID NO: 3500, wherein the CDRs are as defined above. In one embodiment, the ISVD specifically binding to PD-1 comprises or consists of the amino acid sequence of SEQ ID NO: 3500.

In another embodiment, the ISVD specifically binding to human PD-1 may have a sequence identity of more than 90%, such as more than 95% or even more than 99%, with SEQ ID NO: 3501, wherein the CDRs are as defined above. In one embodiment, the ISVD specifically binding to PD-1 comprises or consists of the amino acid sequence of SEQ ID NO: 3501.

In another embodiment, the ISVD specifically binding to human PD-1 may have a sequence identity of more than 90%, such as more than 95% or even more than 99%, with SEQ ID NO: 3502, wherein the CDRs are as defined above. In one embodiment, the ISVD specifically binding to PD-1 comprises or consists of the amino acid sequence of SEQ ID NO: 3502.

In another embodiment, the ISVD specifically binding to human PD-1 may have a sequence identity of more than 90%, such as more than 95% or even more than 99%, with SEQ ID NO: 3503, wherein the CDRs are as defined above. In one embodiment, the ISVD specifically binding to PD-1 comprises or consists of the amino acid sequence of SEQ ID NO: 3503.

In another embodiment, the ISVD specifically binding to human PD-1 may have a sequence identity of more than 90%, such as more than 95% or even more than 99%, with SEQ ID NO: 3504, wherein the CDRs are as defined above. In one embodiment, the ISVD specifically binding to PD-1 comprises or consists of the amino acid sequence of SEQ ID NO: 3504.

In another embodiment, the ISVD specifically binding to human PD-1 may have a sequence identity of more than 90%, such as more than 95% or even more than 99%, with SEQ ID NO: 3505, wherein the CDRs are as defined above. In one embodiment, the ISVD specifically binding to PD-1 comprises or consists of the amino acid sequence of SEQ ID NO: 3505.

In another embodiment, the ISVD specifically binding to human PD-1 may have a sequence identity of more than 90%, such as more than 95% or even more than 99%, with SEQ ID NO: 3506, wherein the CDRs are as defined above. In one embodiment, the ISVD specifically binding to PD-1 comprises or consists of the amino acid sequence of SEQ ID NO: 3506.

In another embodiment, the ISVD specifically binding to human PD-1 may have a sequence identity of more than 90%, such as more than 95% or even more than 99%, with SEQ ID NO: 3507, wherein the CDRs are as defined above. In one embodiment, the ISVD specifically binding to PD-1 comprises or consists of the amino acid sequence of SEQ ID NO: 3507.

In another embodiment, the ISVD specifically binding to human PD-1 may have a sequence identity of more than 90%, such as more than 95% or even more than 99%, with SEQ ID NO: 3508, wherein the CDRs are as defined above. In one embodiment, the ISVD specifically binding to PD-1 comprises or consists of the amino acid sequence of SEQ ID NO: 3508.

In another embodiment, the ISVD specifically binding to human PD-1 may have a sequence identity of more than 90%, such as more than 95% or even more than 99%, with SEQ ID NO: 3509, wherein the CDRs are as defined above. In one embodiment, the ISVD specifically binding to PD-1 comprises or consists of the amino acid sequence of SEQ ID NO: 3509.

In another embodiment, the ISVD specifically binding to human PD-1 may have a sequence identity of more than 90%, such as more than 95% or even more than 99%, with SEQ ID NO: 3510, wherein the CDRs are as defined above. In one embodiment, the ISVD specifically binding to PD-1 comprises or consists of the amino acid sequence of SEQ ID NO: 3510.

In another embodiment, the ISVD specifically binding to human PD-1 may have a sequence identity of more than 90%, such as more than 95% or even more than 99%, with SEQ ID NO: 3511, wherein the CDRs are as defined above. In one embodiment, the ISVD specifically binding to PD-1 comprises or consists of the amino acid sequence of SEQ ID NO: 3511.

In another embodiment, the ISVD specifically binding to human PD-1 may have a sequence identity of more than 90%, such as more than 95% or even more than 99%, with SEQ ID NO: 3512, wherein the CDRs are as defined above. In one embodiment, the ISVD specifically binding to PD-1 comprises or consists of the amino acid sequence of SEQ ID NO: 3512.

In another embodiment, the ISVD specifically binding to human PD-1 may have a sequence identity of more than 90%, such as more than 95% or even more than 99%, with SEQ ID NO: 3513, wherein the CDRs are as defined above. In one embodiment, the ISVD specifically binding to PD-1 comprises or consists of the amino acid sequence of SEQ ID NO: 3513.

In another embodiment, the ISVD specifically binding to human PD-1 may have a sequence identity of more than 90%, such as more than 95% or even more than 99%, with SEQ ID NO: 3514, wherein the CDRs are as defined above. In one embodiment, the ISVD specifically binding to PD-1 comprises or consists of the amino acid sequence of SEQ ID NO: 3514.

In another embodiment, the ISVD specifically binding to human PD-1 may have a sequence identity of more than 90%, such as more than 95% or even more than 99%, with SEQ ID NO: 3515, wherein the CDRs are as defined above. In one embodiment, the ISVD specifically binding to PD-1 comprises or consists of the amino acid sequence of SEQ ID NO: 3515.

In another embodiment, the ISVD specifically binding to human PD-1 may have a sequence identity of more than 90%, such as more than 95% or even more than 99%, with SEQ ID NO: 3516, wherein the CDRs are as defined above. In one embodiment, the ISVD specifically binding to PD-1 comprises or consists of the amino acid sequence of SEQ ID NO: 3516.

In another embodiment, the ISVD specifically binding to human PD-1 may have a sequence identity of more than 90%, such as more than 95% or even more than 99%, with SEQ ID NO: 3517, wherein the CDRs are as defined above. In one embodiment, the ISVD specifically binding to PD-1 comprises or consists of the amino acid sequence of SEQ ID NO: 3517.

In another embodiment, the ISVD specifically binding to human PD-1 may have a sequence identity of more than 90%, such as more than 95% or even more than 99%, with SEQ ID NO: 3518, wherein the CDRs are as defined above. In one embodiment, the ISVD specifically binding to PD-1 comprises or consists of the amino acid sequence of SEQ ID NO: 3518.

In another embodiment, the ISVD specifically binding to human PD-1 may have a sequence identity of more than 90%, such as more than 95% or even more than 99%, with SEQ ID NO: 3519, wherein the CDRs are as defined above. In one embodiment, the ISVD specifically binding to PD-1 comprises or consists of the amino acid sequence of SEQ ID NO: 3519.

In another embodiment, the ISVD specifically binding to human PD-1 may have a sequence identity of more than 90%, such as more than 95% or even more than 99%, with SEQ ID NO: 3520, wherein the CDRs are as defined above. In one embodiment, the ISVD specifically binding to PD-1 comprises or consists of the amino acid sequence of SEQ ID NO: 3520.

In another embodiment, the ISVD specifically binding to human PD-1 may have a sequence identity of more than 90%, such as more than 95% or even more than 99%, with SEQ ID NO: 3521, wherein the CDRs are as defined above. In one embodiment, the ISVD specifically binding to PD-1 comprises or consists of the amino acid sequence of SEQ ID NO: 3521.

In another embodiment, the ISVD specifically binding to human PD-1 may have a sequence identity of more than 90%, such as more than 95% or even more than 99%, with SEQ ID NO: 3522, wherein the CDRs are as defined above. In one embodiment, the ISVD specifically binding to PD-1 comprises or consists of the amino acid sequence of SEQ ID NO: 3522.

In another embodiment, the ISVD specifically binding to human PD-1 may have a sequence identity of more than 90%, such as more than 95% or even more than 99%, with SEQ ID NO: 3523, wherein the CDRs are as defined above. In one embodiment, the ISVD specifically binding to PD-1 comprises or consists of the amino acid sequence of SEQ ID NO: 3523.

In another embodiment, the ISVD specifically binding to human PD-1 may have a sequence identity of more than 90%, such as more than 95% or even more than 99%, with SEQ ID NO: 3524, wherein the CDRs are as defined above. In one embodiment, the ISVD specifically binding to PD-1 comprises or consists of the amino acid sequence of SEQ ID NO: 3524.

In another embodiment, the ISVD specifically binding to human PD-1 may have a sequence identity of more than 90%, such as more than 95% or even more than 99%, with SEQ ID NO: 3525, wherein the CDRs are as defined above. In one embodiment, the ISVD specifically binding to PD-1 comprises or consists of the amino acid sequence of SEQ ID NO: 3525.

In another embodiment, the ISVD specifically binding to human PD-1 may have a sequence identity of more than 90%, such as more than 95% or even more than 99%, with SEQ ID NO: 3526, wherein the CDRs are as defined above. In one embodiment, the ISVD specifically binding to PD-1 comprises or consists of the amino acid sequence of SEQ ID NO: 3526.

In another embodiment, the ISVD specifically binding to human PD-1 may have a sequence identity of more than 90%, such as more than 95% or even more than 99%, with SEQ ID NO: 3527, wherein the CDRs are as defined above. In one embodiment, the ISVD specifically binding to PD-1 comprises or consists of the amino acid sequence of SEQ ID NO: 3527.

In another embodiment, the ISVD specifically binding to human PD-1 may have a sequence identity of more than 90%, such as more than 95% or even more than 99%, with SEQ ID NO: 3528, wherein the CDRs are as defined above. In one embodiment, the ISVD specifically binding to PD-1 comprises or consists of the amino acid sequence of SEQ ID NO: 3528.

In another embodiment, the ISVD specifically binding to human PD-1 may have a sequence identity of more than 90%, such as more than 95% or even more than 99%, with SEQ ID NO: 3529, wherein the CDRs are as defined above. In one embodiment, the ISVD specifically binding to PD-1 comprises or consists of the amino acid sequence of SEQ ID NO: 3529.

In another embodiment, the ISVD specifically binding to human PD-1 may have a sequence identity of more than 90%, such as more than 95% or even more than 99%, with SEQ ID NO: 3530, wherein the CDRs are as defined above. In one embodiment, the ISVD specifically binding to PD-1 comprises or consists of the amino acid sequence of SEQ ID NO: 3530.

In another embodiment, the ISVD specifically binding to human PD-1 may have a sequence identity of more than 90%, such as more than 95% or even more than 99%, with SEQ ID NO: 3531, wherein the CDRs are as defined above. In one embodiment, the ISVD specifically binding to PD-1 comprises or consists of the amino acid sequence of SEQ ID NO: 3531.

In another embodiment, the ISVD specifically binding to human PD-1 may have a sequence identity of more than 90%, such as more than 95% or even more than 99%, with SEQ ID NO: 3532, wherein the CDRs are as defined above. In one embodiment, the ISVD specifically binding to PD-1 comprises or consists of the amino acid sequence of SEQ ID NO: 3532.

In another embodiment, the ISVD specifically binding to human PD-1 may have a sequence identity of more than 90%, such as more than 95% or even more than 99%, with SEQ ID NO: 3533, wherein the CDRs are as defined above. In one embodiment, the ISVD specifically binding to PD-1 comprises or consists of the amino acid sequence of SEQ ID NO: 3533.

In another embodiment, the ISVD specifically binding to human PD-1 may have a sequence identity of more than 90%, such as more than 95% or even more than 99%, with SEQ ID NO: 3534, wherein the CDRs are as defined above. In one embodiment, the ISVD specifically binding to PD-1 comprises or consists of the amino acid sequence of SEQ ID NO: 3534.

In another embodiment, the ISVD specifically binding to human PD-1 may have a sequence identity of more than 90%, such as more than 95% or even more than 99%, with SEQ ID NO: 3535, wherein the CDRs are as defined above. In one embodiment, the ISVD specifically binding to PD-1 comprises or consists of the amino acid sequence of SEQ ID NO: 3535.

In another embodiment, the ISVD specifically binding to human PD-1 may have a sequence identity of more than 90%, such as more than 95% or even more than 99%, with SEQ ID NO: 3536, wherein the CDRs are as defined above. In one embodiment, the ISVD specifically binding to PD-1 comprises or consists of the amino acid sequence of SEQ ID NO: 3536.

In another embodiment, the ISVD specifically binding to human PD-1 may have a sequence identity of more than 90%, such as more than 95% or even more than 99%, with SEQ ID NO: 3537, wherein the CDRs are as defined above. In one embodiment, the ISVD specifically binding to PD-1 comprises or consists of the amino acid sequence of SEQ ID NO: 3537.

In another embodiment, the ISVD specifically binding to human PD-1 may have a sequence identity of more than 90%, such as more than 95% or even more than 99%, with SEQ ID NO: 3538, wherein the CDRs are as defined above. In one embodiment, the ISVD specifically binding to PD-1 comprises or consists of the amino acid sequence of SEQ ID NO: 3538.

In another embodiment, the ISVD specifically binding to human PD-1 may have a sequence identity of more than 90%, such as more than 95% or even more than 99%, with SEQ ID NO: 3539, wherein the CDRs are as defined above. In one embodiment, the ISVD specifically binding to PD-1 comprises or consists of the amino acid sequence of SEQ ID NO: 3539.

In another embodiment, the ISVD specifically binding to human PD-1 may have a sequence identity of more than 90%, such as more than 95% or even more than 99%, with SEQ ID NO: 3540, wherein the CDRs are as defined above. In one embodiment, the ISVD specifically binding to PD-1 comprises or consists of the amino acid sequence of SEQ ID NO: 3540.

In another embodiment, the ISVD specifically binding to human PD-1 may have a sequence identity of more than 90%, such as more than 95% or even more than 99%, with SEQ ID NO: 3541, wherein the CDRs are as defined above. In one embodiment, the ISVD specifically binding to PD-1 comprises or consists of the amino acid sequence of SEQ ID NO: 3541.

In another embodiment, the ISVD specifically binding to human PD-1 may have a sequence identity of more than 90%, such as more than 95% or even more than 99%, with SEQ ID NO: 3542, wherein the CDRs are as defined above. In one embodiment, the ISVD specifically binding to PD-1 comprises or consists of the amino acid sequence of SEQ ID NO: 3542.

In another embodiment, the ISVD specifically binding to human PD-1 may have a sequence identity of more than 90%, such as more than 95% or even more than 99%, with SEQ ID NO: 3543, wherein the CDRs are as defined above. In one embodiment, the ISVD specifically binding to PD-1 comprises or consists of the amino acid sequence of SEQ ID NO: 3543.

In another embodiment, the ISVD specifically binding to human PD-1 may have a sequence identity of more than 90%, such as more than 95% or even more than 99%, with SEQ ID NO: 3544, wherein the CDRs are as defined above. In one embodiment, the ISVD specifically binding to PD-1 comprises or consists of the amino acid sequence of SEQ ID NO: 3544.

In another embodiment, the ISVD specifically binding to human PD-1 may have a sequence identity of more than 90%, such as more than 95% or even more than 99%, with SEQ ID NO: 3545, wherein the CDRs are as defined above. In one embodiment, the ISVD specifically binding to PD-1 comprises or consists of the amino acid sequence of SEQ ID NO: 3545.

In another embodiment, the ISVD specifically binding to human PD-1 may have a sequence identity of more than 90%, such as more than 95% or even more than 99%, with SEQ ID NO: 3546, wherein the CDRs are as defined above. In one embodiment, the ISVD specifically binding to PD-1 comprises or consists of the amino acid sequence of SEQ ID NO: 3546.

In another embodiment, the ISVD specifically binding to human PD-1 may have a sequence identity of more than 90%, such as more than 95% or even more than 99%, with SEQ ID NO: 3547, wherein the CDRs are as defined above. In one embodiment, the ISVD specifically binding to PD-1 comprises or consists of the amino acid sequence of SEQ ID NO: 3547.

In another embodiment, the ISVD specifically binding to human PD-1 may have a sequence identity of more than 90%, such as more than 95% or even more than 99%, with SEQ ID NO: 3548, wherein the CDRs are as defined above. In one embodiment, the ISVD specifically binding to PD-1 comprises or consists of the amino acid sequence of SEQ ID NO: 3548.

In another embodiment, the ISVD specifically binding to human PD-1 may have a sequence identity of more than 90%, such as more than 95% or even more than 99%, with SEQ ID NO: 3549, wherein the CDRs are as defined above. In one embodiment, the ISVD specifically binding to PD-1 comprises or consists of the amino acid sequence of SEQ ID NO: 3549.

In another embodiment, the ISVD specifically binding to human PD-1 may have a sequence identity of more than 90%, such as more than 95% or even more than 99%, with SEQ ID NO: 3550, wherein the CDRs are as defined above. In one embodiment, the ISVD specifically binding to PD-1 comprises or consists of the amino acid sequence of SEQ ID NO: 3550.

In another embodiment, the ISVD specifically binding to human PD-1 may have a sequence identity of more than 90%, such as more than 95% or even more than 99%, with SEQ ID NO: 3551, wherein the CDRs are as defined above. In one embodiment, the ISVD specifically binding to PD-1 comprises or consists of the amino acid sequence of SEQ ID NO: 3551.

In another embodiment, the ISVD specifically binding to human PD-1 may have a sequence identity of more than 90%, such as more than 95% or even more than 99%, with SEQ ID NO: 3552, wherein the CDRs are as defined above. In one embodiment, the ISVD specifically binding to PD-1 comprises or consists of the amino acid sequence of SEQ ID NO: 3552.

In another embodiment, the ISVD specifically binding to human PD-1 may have a sequence identity of more than 90%, such as more than 95% or even more than 99%, with SEQ ID NO: 3553, wherein the CDRs are as defined above. In one embodiment, the ISVD specifically binding to PD-1 comprises or consists of the amino acid sequence of SEQ ID NO: 3553.

In another embodiment, the ISVD specifically binding to human PD-1 may have a sequence identity of more than 90%, such as more than 95% or even more than 99%, with SEQ ID NO: 3554, wherein the CDRs are as defined above. In one embodiment, the ISVD specifically binding to PD-1 comprises or consists of the amino acid sequence of SEQ ID NO: 3554.

In another embodiment, the ISVD specifically binding to human PD-1 may have a sequence identity of more than 90%, such as more than 95% or even more than 99%, with SEQ ID NO: 3555, wherein the CDRs are as defined above. In one embodiment, the ISVD specifically binding to PD-1 comprises or consists of the amino acid sequence of SEQ ID NO: 3555.

In another embodiment, the ISVD specifically binding to human PD-1 may have a sequence identity of more than 90%, such as more than 95% or even more than 99%, with SEQ ID NO: 3556, wherein the CDRs are as defined above. In one embodiment, the ISVD specifically binding to PD-1 comprises or consists of the amino acid sequence of SEQ ID NO: 3556.

In another embodiment, the ISVD specifically binding to human PD-1 may have a sequence identity of more than 90%, such as more than 95% or even more than 99%, with SEQ ID NO: 3557, wherein the CDRs are as defined above. In one embodiment, the ISVD specifically binding to PD-1 comprises or consists of the amino acid sequence of SEQ ID NO: 3557.

In another embodiment, the ISVD specifically binding to human PD-1 may have a sequence identity of more than 90%, such as more than 95% or even more than 99%, with SEQ ID NO: 3558, wherein the CDRs are as defined above. In one embodiment, the ISVD specifically binding to PD-1 comprises or consists of the amino acid sequence of SEQ ID NO: 3558.

In another embodiment, the ISVD specifically binding to human PD-1 may have a sequence identity of more than 90%, such as more than 95% or even more than 99%, with SEQ ID NO: 3559, wherein the CDRs are as defined above. In one embodiment, the ISVD specifically binding to PD-1 comprises or consists of the amino acid sequence of SEQ ID NO: 3559.

In another embodiment, the ISVD specifically binding to human PD-1 may have a sequence identity of more than 90%, such as more than 95% or even more than 99%, with SEQ ID NO: 3560, wherein the CDRs are as defined above. In one embodiment, the ISVD specifically binding to PD-1 comprises or consists of the amino acid sequence of SEQ ID NO: 3560.

In another embodiment, the ISVD specifically binding to human PD-1 may have a sequence identity of more than 90%, such as more than 95% or even more than 99%, with SEQ ID NO: 3561, wherein the CDRs are as defined above. In one embodiment, the ISVD specifically binding to PD-1 comprises or consists of the amino acid sequence of SEQ ID NO: 3561.

In another embodiment, the ISVD specifically binding to human PD-1 may have a sequence identity of more than 90%, such as more than 95% or even more than 99%, with SEQ ID NO: 3562, wherein the CDRs are as defined above. In one embodiment, the ISVD specifically binding to PD-1 comprises or consists of the amino acid sequence of SEQ ID NO: 3562.

In another embodiment, the ISVD specifically binding to human PD-1 may have a sequence identity of more than 90%, such as more than 95% or even more than 99%, with SEQ ID NO: 3563, wherein the CDRs are as defined above. In one embodiment, the ISVD specifically binding to PD-1 comprises or consists of the amino acid sequence of SEQ ID NO: 3563.

In another embodiment, the ISVD specifically binding to human PD-1 may have a sequence identity of more than 90%, such as more than 95% or even more than 99%, with SEQ ID NO: 3564, wherein the CDRs are as defined above. In one embodiment, the ISVD specifically binding to PD-1 comprises or consists of the amino acid sequence of SEQ ID NO: 3564.

In another embodiment, the ISVD specifically binding to human PD-1 may have a sequence identity of more than 90%, such as more than 95% or even more than 99%, with SEQ ID NO: 3565, wherein the CDRs are as defined above. In one embodiment, the ISVD specifically binding to PD-1 comprises or consists of the amino acid sequence of SEQ ID NO: 3565.

In another embodiment, the ISVD specifically binding to human PD-1 may have a sequence identity of more than 90%, such as more than 95% or even more than 99%, with SEQ ID NO: 3566, wherein the CDRs are as defined above. In one embodiment, the ISVD specifically binding to PD-1 comprises or consists of the amino acid sequence of SEQ ID NO: 3566.

In another embodiment, the ISVD specifically binding to human PD-1 may have a sequence identity of more than 90%, such as more than 95% or even more than 99%, with SEQ ID NO: 3567, wherein the CDRs are as defined above. In one embodiment, the ISVD specifically binding to PD-1 comprises or consists of the amino acid sequence of SEQ ID NO: 3567.

In another embodiment, the ISVD specifically binding to human PD-1 may have a sequence identity of more than 90%, such as more than 95% or even more than 99%, with SEQ ID NO: 3568, wherein the CDRs are as defined above. In one embodiment, the ISVD specifically binding to PD-1 comprises or consists of the amino acid sequence of SEQ ID NO: 3568.

In another embodiment, the ISVD specifically binding to human PD-1 may have a sequence identity of more than 90%, such as more than 95% or even more than 99%, with SEQ ID NO: 3569, wherein the CDRs are as defined above. In one embodiment, the ISVD specifically binding to PD-1 comprises or consists of the amino acid sequence of SEQ ID NO: 3569.

In another embodiment, the ISVD specifically binding to human PD-1 may have a sequence identity of more than 90%, such as more than 95% or even more than 99%, with SEQ ID NO: 3570, wherein the CDRs are as defined above. In one embodiment, the ISVD specifically binding to PD-1 comprises or consists of the amino acid sequence of SEQ ID NO: 3570.

In another embodiment, the ISVD specifically binding to human PD-1 may have a sequence identity of more than 90%, such as more than 95% or even more than 99%, with SEQ ID NO: 3571, wherein the CDRs are as defined above. In one embodiment, the ISVD specifically binding to PD-1 comprises or consists of the amino acid sequence of SEQ ID NO: 3571.

In another embodiment, the ISVD specifically binding to human PD-1 may have a sequence identity of more than 90%, such as more than 95% or even more than 99%, with SEQ ID NO: 3572, wherein the CDRs are as defined above. In one embodiment, the ISVD specifically binding to PD-1 comprises or consists of the amino acid sequence of SEQ ID NO: 3572.

In another embodiment, the ISVD specifically binding to human PD-1 may have a sequence identity of more than 90%, such as more than 95% or even more than 99%, with SEQ ID NO: 3573, wherein the CDRs are as defined above. In one embodiment, the ISVD specifically binding to PD-1 comprises or consists of the amino acid sequence of SEQ ID NO: 3573.

In another embodiment, the ISVD specifically binding to human PD-1 may have a sequence identity of more than 90%, such as more than 95% or even more than 99%, with SEQ ID NO: 3574, wherein the CDRs are as defined above. In one embodiment, the ISVD specifically binding to PD-1 comprises or consists of the amino acid sequence of SEQ ID NO: 3574.

In another embodiment, the ISVD specifically binding to human PD-1 may have a sequence identity of more than 90%, such as more than 95% or even more than 99%, with SEQ ID NO: 3575, wherein the CDRs are as defined above. In one embodiment, the ISVD specifically binding to PD-1 comprises or consists of the amino acid sequence of SEQ ID NO: 3575.

In another embodiment, the ISVD specifically binding to human PD-1 may have a sequence identity of more than 90%, such as more than 95% or even more than 99%, with SEQ ID NO: 3576, wherein the CDRs are as defined above. In one embodiment, the ISVD specifically binding to PD-1 comprises or consists of the amino acid sequence of SEQ ID NO: 3576.

In another embodiment, the ISVD specifically binding to human PD-1 may have a sequence identity of more than 90%, such as more than 95% or even more than 99%, with SEQ ID NO: 3577, wherein the CDRs are as defined above. In one embodiment, the ISVD specifically binding to PD-1 comprises or consists of the amino acid sequence of SEQ ID NO: 3577.

In another embodiment, the ISVD specifically binding to human PD-1 may have a sequence identity of more than 90%, such as more than 95% or even more than 99%, with SEQ ID NO: 3578, wherein the CDRs are as defined above. In one embodiment, the ISVD specifically binding to PD-1 comprises or consists of the amino acid sequence of SEQ ID NO: 3578.

In another embodiment, the ISVD specifically binding to human PD-1 may have a sequence identity of more than 90%, such as more than 95% or even more than 99%, with SEQ ID NO: 3579, wherein the CDRs are as defined above. In one embodiment, the ISVD specifically binding to PD-1 comprises or consists of the amino acid sequence of SEQ ID NO: 3579.

In another embodiment, the ISVD specifically binding to human PD-1 may have a sequence identity of more than 90%, such as more than 95% or even more than 99%, with SEQ ID NO: 3580, wherein the CDRs are as defined above. In one embodiment, the ISVD specifically binding to PD-1 comprises or consists of the amino acid sequence of SEQ ID NO: 3580.

In another embodiment, the ISVD specifically binding to human PD-1 may have a sequence identity of more than 90%, such as more than 95% or even more than 99%, with SEQ ID NO: 3581, wherein the CDRs are as defined above. In one embodiment, the ISVD specifically binding to PD-1 comprises or consists of the amino acid sequence of SEQ ID NO: 3581.

In another embodiment, the ISVD specifically binding to human PD-1 may have a sequence identity of more than 90%, such as more than 95% or even more than 99%, with SEQ ID NO: 3582, wherein the CDRs are as defined above. In one embodiment, the ISVD specifically binding to PD-1 comprises or consists of the amino acid sequence of SEQ ID NO: 3582.

In another embodiment, the ISVD specifically binding to human PD-1 may have a sequence identity of more than 90%, such as more than 95% or even more than 99%, with SEQ ID NO: 3583, wherein the CDRs are as defined above. In one embodiment, the ISVD specifically binding to PD-1 comprises or consists of the amino acid sequence of SEQ ID NO: 3583.

In another embodiment, the ISVD specifically binding to human PD-1 may have a sequence identity of more than 90%, such as more than 95% or even more than 99%, with SEQ ID NO: 3584, wherein the CDRs are as defined above. In one embodiment, the ISVD specifically binding to PD-1 comprises or consists of the amino acid sequence of SEQ ID NO: 3584.

In another embodiment, the ISVD specifically binding to human PD-1 may have a sequence identity of more than 90%, such as more than 95% or even more than 99%, with SEQ ID NO: 3585, wherein the CDRs are as defined above. In one embodiment, the ISVD specifically binding to PD-1 comprises or consists of the amino acid sequence of SEQ ID NO: 3585.

In another embodiment, the ISVD specifically binding to human PD-1 may have a sequence identity of more than 90%, such as more than 95% or even more than 99%, with SEQ ID NO: 3586, wherein the CDRs are as defined above. In one embodiment, the ISVD specifically binding to PD-1 comprises or consists of the amino acid sequence of SEQ ID NO: 3586.

In another embodiment, the ISVD specifically binding to human PD-1 may have a sequence identity of more than 90%, such as more than 95% or even more than 99%, with SEQ ID NO: 3587, wherein the CDRs are as defined above. In one embodiment, the ISVD specifically binding to PD-1 comprises or consists of the amino acid sequence of SEQ ID NO: 3587.

In another embodiment, the ISVD specifically binding to human PD-1 may have a sequence identity of more than 90%, such as more than 95% or even more than 99%, with SEQ ID NO: 3588, wherein the CDRs are as defined above. In one embodiment, the ISVD specifically binding to PD-1 comprises or consists of the amino acid sequence of SEQ ID NO: 3588.

In another embodiment, the ISVD specifically binding to human PD-1 may have a sequence identity of more than 90%, such as more than 95% or even more than 99%, with SEQ ID NO: 3589, wherein the CDRs are as defined above. In one embodiment, the ISVD specifically binding to PD-1 comprises or consists of the amino acid sequence of SEQ ID NO: 3589.

In another embodiment, the ISVD specifically binding to human PD-1 may have a sequence identity of more than 90%, such as more than 95% or even more than 99%, with SEQ ID NO: 3590, wherein the CDRs are as defined above. In one embodiment, the ISVD specifically binding to PD-1 comprises or consists of the amino acid sequence of SEQ ID NO: 3590.

In another embodiment, the ISVD specifically binding to human PD-1 may have a sequence identity of more than 90%, such as more than 95% or even more than 99%, with SEQ ID NO: 3591, wherein the CDRs are as defined above. In one embodiment, the ISVD specifically binding to PD-1 comprises or consists of the amino acid sequence of SEQ ID NO: 3591.

In another embodiment, the ISVD specifically binding to human PD-1 may have a sequence identity of more than 90%, such as more than 95% or even more than 99%, with SEQ ID NO: 3592, wherein the CDRs are as defined above. In one embodiment, the ISVD specifically binding to PD-1 comprises or consists of the amino acid sequence of SEQ ID NO: 3592.

In another embodiment, the ISVD specifically binding to human PD-1 may have a sequence identity of more than 90%, such as more than 95% or even more than 99%, with SEQ ID NO: 3593, wherein the CDRs are as defined above. In one embodiment, the ISVD specifically binding to PD-1 comprises or consists of the amino acid sequence of SEQ ID NO: 3593.

In another embodiment, the ISVD specifically binding to human PD-1 may have a sequence identity of more than 90%, such as more than 95% or even more than 99%, with SEQ ID NO: 3594, wherein the CDRs are as defined above. In one embodiment, the ISVD specifically binding to PD-1 comprises or consists of the amino acid sequence of SEQ ID NO: 3594.

In another embodiment, the ISVD specifically binding to human PD-1 may have a sequence identity of more than 90%, such as more than 95% or even more than 99%, with SEQ ID NO: 3595, wherein the CDRs are as defined above. In one embodiment, the ISVD specifically binding to PD-1 comprises or consists of the amino acid sequence of SEQ ID NO: 3595.

In another embodiment, the ISVD specifically binding to human PD-1 may have a sequence identity of more than 90%, such as more than 95% or even more than 99%, with SEQ ID NO: 3596, wherein the CDRs are as defined above. In one embodiment, the ISVD specifically binding to PD-1 comprises or consists of the amino acid sequence of SEQ ID NO: 3596.

In another embodiment, the ISVD specifically binding to human PD-1 may have a sequence identity of more than 90%, such as more than 95% or even more than 99%, with SEQ ID NO: 3597, wherein the CDRs are as defined above. In one embodiment, the ISVD specifically binding to PD-1 comprises or consists of the amino acid sequence of SEQ ID NO: 3597.

In another embodiment, the ISVD specifically binding to human PD-1 may have a sequence identity of more than 90%, such as more than 95% or even more than 99%, with SEQ ID NO: 3598, wherein the CDRs are as defined above. In one embodiment, the ISVD specifically binding to PD-1 comprises or consists of the amino acid sequence of SEQ ID NO: 3598.

In another embodiment, the ISVD specifically binding to human PD-1 may have a sequence identity of more than 90%, such as more than 95% or even more than 99%, with SEQ ID NO: 3599, wherein the CDRs are as defined above. In one embodiment, the ISVD specifically binding to PD-1 comprises or consists of the amino acid sequence of SEQ ID NO: 3599.

In another embodiment, the ISVD specifically binding to human PD-1 may have a sequence identity of more than 90%, such as more than 95% or even more than 99%, with SEQ ID NO: 3600, wherein the CDRs are as defined above. In one embodiment, the ISVD specifically binding to PD-1 comprises or consists of the amino acid sequence of SEQ ID NO: 3600.

In another embodiment, the ISVD specifically binding to human PD-1 may have a sequence identity of more than 90%, such as more than 95% or even more than 99%, with SEQ ID NO: 3601, wherein the CDRs are as defined above. In one embodiment, the ISVD specifically binding to PD-1 comprises or consists of the amino acid sequence of SEQ ID NO: 3601.

In another embodiment, the ISVD specifically binding to human PD-1 may have a sequence identity of more than 90%, such as more than 95% or even more than 99%, with SEQ ID NO: 3602, wherein the CDRs are as defined above. In one embodiment, the ISVD specifically binding to PD-1 comprises or consists of the amino acid sequence of SEQ ID NO: 3602.

In another embodiment, the ISVD specifically binding to human PD-1 may have a sequence identity of more than 90%, such as more than 95% or even more than 99%, with SEQ ID NO: 3603, wherein the CDRs are as defined above. In one embodiment, the ISVD specifically binding to PD-1 comprises or consists of the amino acid sequence of SEQ ID NO: 3603.

In another embodiment, the ISVD specifically binding to human PD-1 may have a sequence identity of more than 90%, such as more than 95% or even more than 99%, with SEQ ID NO: 3604, wherein the CDRs are as defined above. In one embodiment, the ISVD specifically binding to PD-1 comprises or consists of the amino acid sequence of SEQ ID NO: 3604.

In another embodiment, the ISVD specifically binding to human PD-1 may have a sequence identity of more than 90%, such as more than 95% or even more than 99%, with SEQ ID NO: 3605, wherein the CDRs are as defined above. In one embodiment, the ISVD specifically binding to PD-1 comprises or consists of the amino acid sequence of SEQ ID NO: 3605.

In another embodiment, the ISVD specifically binding to human PD-1 may have a sequence identity of more than 90%, such as more than 95% or even more than 99%, with SEQ ID NO: 3606, wherein the CDRs are as defined above. In one embodiment, the ISVD specifically binding to PD-1 comprises or consists of the amino acid sequence of SEQ ID NO: 3606.

In another embodiment, the ISVD specifically binding to human PD-1 may have a sequence identity of more than 90%, such as more than 95% or even more than 99%, with SEQ ID NO: 3607, wherein the CDRs are as defined above. In one embodiment, the ISVD specifically binding to PD-1 comprises or consists of the amino acid sequence of SEQ ID NO: 3607.

In another embodiment, the ISVD specifically binding to human PD-1 may have a sequence identity of more than 90%, such as more than 95% or even more than 99%, with SEQ ID NO: 3608, wherein the CDRs are as defined above. In one embodiment, the ISVD specifically binding to PD-1 comprises or consists of the amino acid sequence of SEQ ID NO: 3608.

In another embodiment, the ISVD specifically binding to human PD-1 may have a sequence identity of more than 90%, such as more than 95% or even more than 99%, with SEQ ID NO: 3609, wherein the CDRs are as defined above. In one embodiment, the ISVD specifically binding to PD-1 comprises or consists of the amino acid sequence of SEQ ID NO: 3609.

In another embodiment, the ISVD specifically binding to human PD-1 may have a sequence identity of more than 90%, such as more than 95% or even more than 99%, with SEQ ID NO: 3610, wherein the CDRs are as defined above. In one embodiment, the ISVD specifically binding to PD-1 comprises or consists of the amino acid sequence of SEQ ID NO: 3610.

In another embodiment, the ISVD specifically binding to human PD-1 may have a sequence identity of more than 90%, such as more than 95% or even more than 99%, with SEQ ID NO: 3611, wherein the CDRs are as defined above. In one embodiment, the ISVD specifically binding to PD-1 comprises or consists of the amino acid sequence of SEQ ID NO: 3611.

In another embodiment, the ISVD specifically binding to human PD-1 may have a sequence identity of more than 90%, such as more than 95% or even more than 99%, with SEQ ID NO: 3612, wherein the CDRs are as defined above. In one embodiment, the ISVD specifically binding to PD-1 comprises or consists of the amino acid sequence of SEQ ID NO: 3612.

In another embodiment, the ISVD specifically binding to human PD-1 may have a sequence identity of more than 90%, such as more than 95% or even more than 99%, with SEQ ID NO: 3613, wherein the CDRs are as defined above. In one embodiment, the ISVD specifically binding to PD-1 comprises or consists of the amino acid sequence of SEQ ID NO: 3613.

In another embodiment, the ISVD specifically binding to human PD-1 may have a sequence identity of more than 90%, such as more than 95% or even more than 99%, with SEQ ID NO: 3614, wherein the CDRs are as defined above. In one embodiment, the ISVD specifically binding to PD-1 comprises or consists of the amino acid sequence of SEQ ID NO: 3614.

In another embodiment, the ISVD specifically binding to human PD-1 may have a sequence identity of more than 90%, such as more than 95% or even more than 99%, with SEQ ID NO: 3615, wherein the CDRs are as defined above. In one embodiment, the ISVD specifically binding to PD-1 comprises or consists of the amino acid sequence of SEQ ID NO: 3615.

In another embodiment, the ISVD specifically binding to human PD-1 may have a sequence identity of more than 90%, such as more than 95% or even more than 99%, with SEQ ID NO: 3616, wherein the CDRs are as defined above. In one embodiment, the ISVD specifically binding to PD-1 comprises or consists of the amino acid sequence of SEQ ID NO: 3616.

In another embodiment, the ISVD specifically binding to human PD-1 may have a sequence identity of more than 90%, such as more than 95% or even more than 99%, with SEQ ID NO: 3617, wherein the CDRs are as defined above. In one embodiment, the ISVD specifically binding to PD-1 comprises or consists of the amino acid sequence of SEQ ID NO: 3617.

In another embodiment, the ISVD specifically binding to human PD-1 may have a sequence identity of more than 90%, such as more than 95% or even more than 99%, with SEQ ID NO: 3618, wherein the CDRs are as defined above. In one embodiment, the ISVD specifically binding to PD-1 comprises or consists of the amino acid sequence of SEQ ID NO: 3618.

In another embodiment, the ISVD specifically binding to human PD-1 may have a sequence identity of more than 90%, such as more than 95% or even more than 99%, with SEQ ID NO: 3619, wherein the CDRs are as defined above. In one embodiment, the ISVD specifically binding to PD-1 comprises or consists of the amino acid sequence of SEQ ID NO: 3619.

In another embodiment, the ISVD specifically binding to human PD-1 may have a sequence identity of more than 90%, such as more than 95% or even more than 99%, with SEQ ID NO: 3620, wherein the CDRs are as defined above. In one embodiment, the ISVD specifically binding to PD-1 comprises or consists of the amino acid sequence of SEQ ID NO: 3620.

In another embodiment, the ISVD specifically binding to human PD-1 may have a sequence identity of more than 90%, such as more than 95% or even more than 99%, with SEQ ID NO: 3621, wherein the CDRs are as defined above. In one embodiment, the ISVD specifically binding to PD-1 comprises or consists of the amino acid sequence of SEQ ID NO: 3621.

In another embodiment, the ISVD specifically binding to human PD-1 may have a sequence identity of more than 90%, such as more than 95% or even more than 99%, with SEQ ID NO: 3622, wherein the CDRs are as defined above. In one embodiment, the ISVD specifically binding to PD-1 comprises or consists of the amino acid sequence of SEQ ID NO: 3622.

In another embodiment, the ISVD specifically binding to human PD-1 may have a sequence identity of more than 90%, such as more than 95% or even more than 99%, with SEQ ID NO: 3623, wherein the CDRs are as defined above. In one embodiment, the ISVD specifically binding to PD-1 comprises or consists of the amino acid sequence of SEQ ID NO: 3623.

In another embodiment, the ISVD specifically binding to human PD-1 may have a sequence identity of more than 90%, such as more than 95% or even more than 99%, with SEQ ID NO: 3624, wherein the CDRs are as defined above. In one embodiment, the ISVD specifically binding to PD-1 comprises or consists of the amino acid sequence of SEQ ID NO: 3624.

In another embodiment, the ISVD specifically binding to human PD-1 may have a sequence identity of more than 90%, such as more than 95% or even more than 99%, with SEQ ID NO: 3625, wherein the CDRs are as defined above. In one embodiment, the ISVD specifically binding to PD-1 comprises or consists of the amino acid sequence of SEQ ID NO: 3625.

In another embodiment, the ISVD specifically binding to human PD-1 may have a sequence identity of more than 90%, such as more than 95% or even more than 99%, with SEQ ID NO: 3626, wherein the CDRs are as defined above. In one embodiment, the ISVD specifically binding to PD-1 comprises or consists of the amino acid sequence of SEQ ID NO: 3626.

In another embodiment, the ISVD specifically binding to human PD-1 may have a sequence identity of more than 90%, such as more than 95% or even more than 99%, with SEQ ID NO: 3627, wherein the CDRs are as defined above. In one embodiment, the ISVD specifically binding to PD-1 comprises or consists of the amino acid sequence of SEQ ID NO: 3627.

In another embodiment, the ISVD specifically binding to human PD-1 may have a sequence identity of more than 90%, such as more than 95% or even more than 99%, with SEQ ID NO: 3628, wherein the CDRs are as defined above. In one embodiment, the ISVD specifically binding to PD-1 comprises or consists of the amino acid sequence of SEQ ID NO: 3628.

In another embodiment, the ISVD specifically binding to human PD-1 may have a sequence identity of more than 90%, such as more than 95% or even more than 99%, with SEQ ID NO: 3629, wherein the CDRs are as defined above. In one embodiment, the ISVD specifically binding to PD-1 comprises or consists of the amino acid sequence of SEQ ID NO: 3629.

In another embodiment, the ISVD specifically binding to human PD-1 may have a sequence identity of more than 90%, such as more than 95% or even more than 99%, with SEQ ID NO: 3630, wherein the CDRs are as defined above. In one embodiment, the ISVD specifically binding to PD-1 comprises or consists of the amino acid sequence of SEQ ID NO: 3630.

In another embodiment, the ISVD specifically binding to human PD-1 may have a sequence identity of more than 90%, such as more than 95% or even more than 99%, with SEQ ID NO: 3631, wherein the CDRs are as defined above. In one embodiment, the ISVD specifically binding to PD-1 comprises or consists of the amino acid sequence of SEQ ID NO: 3631.

In another embodiment, the ISVD specifically binding to human PD-1 may have a sequence identity of more than 90%, such as more than 95% or even more than 99%, with SEQ ID NO: 3632, wherein the CDRs are as defined above. In one embodiment, the ISVD specifically binding to PD-1 comprises or consists of the amino acid sequence of SEQ ID NO: 3632.

In another embodiment, the ISVD specifically binding to human PD-1 may have a sequence identity of more than 90%, such as more than 95% or even more than 99%, with SEQ ID NO: 3633, wherein the CDRs are as defined above. In one embodiment, the ISVD specifically binding to PD-1 comprises or consists of the amino acid sequence of SEQ ID NO: 3633.

In another embodiment, the ISVD specifically binding to human PD-1 may have a sequence identity of more than 90%, such as more than 95% or even more than 99%, with SEQ ID NO: 3634, wherein the CDRs are as defined above. In one embodiment, the ISVD specifically binding to PD-1 comprises or consists of the amino acid sequence of SEQ ID NO: 3634.

In another embodiment, the ISVD specifically binding to human PD-1 may have a sequence identity of more than 90%, such as more than 95% or even more than 99%, with SEQ ID NO: 3635, wherein the CDRs are as defined above. In one embodiment, the ISVD specifically binding to PD-1 comprises or consists of the amino acid sequence of SEQ ID NO: 3635.

In another embodiment, the ISVD specifically binding to human PD-1 may have a sequence identity of more than 90%, such as more than 95% or even more than 99%, with SEQ ID NO: 3636, wherein the CDRs are as defined above. In one embodiment, the ISVD specifically binding to PD-1 comprises or consists of the amino acid sequence of SEQ ID NO: 3636.

In another embodiment, the ISVD specifically binding to human PD-1 may have a sequence identity of more than 90%, such as more than 95% or even more than 99%, with SEQ ID NO: 3637, wherein the CDRs are as defined above. In one embodiment, the ISVD specifically binding to PD-1 comprises or consists of the amino acid sequence of SEQ ID NO: 3637.

In another embodiment, the ISVD specifically binding to human PD-1 may have a sequence identity of more than 90%, such as more than 95% or even more than 99%, with SEQ ID NO: 3638, wherein the CDRs are as defined above. In one embodiment, the ISVD specifically binding to PD-1 comprises or consists of the amino acid sequence of SEQ ID NO: 3638.

In another embodiment, the ISVD specifically binding to human PD-1 may have a sequence identity of more than 90%, such as more than 95% or even more than 99%, with SEQ ID NO: 3639, wherein the CDRs are as defined above. In one embodiment, the ISVD specifically binding to PD-1 comprises or consists of the amino acid sequence of SEQ ID NO: 3639.

In another embodiment, the ISVD specifically binding to human PD-1 may have a sequence identity of more than 90%, such as more than 95% or even more than 99%, with SEQ ID NO: 3640, wherein the CDRs are as defined above. In one embodiment, the ISVD specifically binding to PD-1 comprises or consists of the amino acid sequence of SEQ ID NO: 3640.

In another embodiment, the ISVD specifically binding to human PD-1 may have a sequence identity of more than 90%, such as more than 95% or even more than 99%, with SEQ ID NO: 3641, wherein the CDRs are as defined above. In one embodiment, the ISVD specifically binding to PD-1 comprises or consists of the amino acid sequence of SEQ ID NO: 3641.

In another embodiment, the ISVD specifically binding to human PD-1 may have a sequence identity of more than 90%, such as more than 95% or even more than 99%, with SEQ ID NO: 3642, wherein the CDRs are as defined above. In one embodiment, the ISVD specifically binding to PD-1 comprises or consists of the amino acid sequence of SEQ ID NO: 3642.

In another embodiment, the ISVD specifically binding to human PD-1 may have a sequence identity of more than 90%, such as more than 95% or even more than 99%, with SEQ ID NO: 3643, wherein the CDRs are as defined above. In one embodiment, the ISVD specifically binding to PD-1 comprises or consists of the amino acid sequence of SEQ ID NO: 3643.

In another embodiment, the ISVD specifically binding to human PD-1 may have a sequence identity of more than 90%, such as more than 95% or even more than 99%, with SEQ ID NO: 3644, wherein the CDRs are as defined above. In one embodiment, the ISVD specifically binding to PD-1 comprises or consists of the amino acid sequence of SEQ ID NO: 3644.

In another embodiment, the ISVD specifically binding to human PD-1 may have a sequence identity of more than 90%, such as more than 95% or even more than 99%, with SEQ ID NO: 3645, wherein the CDRs are as defined above. In one embodiment, the ISVD specifically binding to PD-1 comprises or consists of the amino acid sequence of SEQ ID NO: 3645.

In another embodiment, the ISVD specifically binding to human PD-1 may have a sequence identity of more than 90%, such as more than 95% or even more than 99%, with SEQ ID NO: 3646, wherein the CDRs are as defined above. In one embodiment, the ISVD specifically binding to PD-1 comprises or consists of the amino acid sequence of SEQ ID NO: 3646.

In another embodiment, the ISVD specifically binding to human PD-1 may have a sequence identity of more than 90%, such as more than 95% or even more than 99%, with SEQ ID NO: 3647, wherein the CDRs are as defined above. In one embodiment, the ISVD specifically binding to PD-1 comprises or consists of the amino acid sequence of SEQ ID NO: 3647.

In another embodiment, the ISVD specifically binding to human PD-1 may have a sequence identity of more than 90%, such as more than 95% or even more than 99%, with SEQ ID NO: 3648, wherein the CDRs are as defined above. In one embodiment, the ISVD specifically binding to PD-1 comprises or consists of the amino acid sequence of SEQ ID NO: 3648.

In another embodiment, the ISVD specifically binding to human PD-1 may have a sequence identity of more than 90%, such as more than 95% or even more than 99%, with SEQ ID NO: 3649, wherein the CDRs are as defined above. In one embodiment, the ISVD specifically binding to PD-1 comprises or consists of the amino acid sequence of SEQ ID NO: 3649.

In another embodiment, the ISVD specifically binding to human PD-1 may have a sequence identity of more than 90%, such as more than 95% or even more than 99%, with SEQ ID NO: 3650, wherein the CDRs are as defined above. In one embodiment, the ISVD specifically binding to PD-1 comprises or consists of the amino acid sequence of SEQ ID NO: 3650.

In another embodiment, the ISVD specifically binding to human PD-1 may have a sequence identity of more than 90%, such as more than 95% or even more than 99%, with SEQ ID NO: 3651, wherein the CDRs are as defined above. In one embodiment, the ISVD specifically binding to PD-1 comprises or consists of the amino acid sequence of SEQ ID NO: 3651.

In another embodiment, the ISVD specifically binding to human PD-1 may have a sequence identity of more than 90%, such as more than 95% or even more than 99%, with SEQ ID NO: 3652, wherein the CDRs are as defined above. In one embodiment, the ISVD specifically binding to PD-1 comprises or consists of the amino acid sequence of SEQ ID NO: 3652.

In another embodiment, the ISVD specifically binding to human PD-1 may have a sequence identity of more than 90%, such as more than 95% or even more than 99%, with SEQ ID NO: 3653, wherein the CDRs are as defined above. In one embodiment, the ISVD specifically binding to PD-1 comprises or consists of the amino acid sequence of SEQ ID NO: 3653.

In another embodiment, the ISVD specifically binding to human PD-1 may have a sequence identity of more than 90%, such as more than 95% or even more than 99%, with SEQ ID NO: 3654, wherein the CDRs are as defined above. In one embodiment, the ISVD specifically binding to PD-1 comprises or consists of the amino acid sequence of SEQ ID NO: 3654.

In another embodiment, the ISVD specifically binding to human PD-1 may have a sequence identity of more than 90%, such as more than 95% or even more than 99%, with SEQ ID NO: 3655, wherein the CDRs are as defined above. In one embodiment, the ISVD specifically binding to PD-1 comprises or consists of the amino acid sequence of SEQ ID NO: 3655.

In another embodiment, the ISVD specifically binding to human PD-1 may have a sequence identity of more than 90%, such as more than 95% or even more than 99%, with SEQ ID NO: 3656, wherein the CDRs are as defined above. In one embodiment, the ISVD specifically binding to PD-1 comprises or consists of the amino acid sequence of SEQ ID NO: 3656.

In another embodiment, the ISVD specifically binding to human PD-1 may have a sequence identity of more than 90%, such as more than 95% or even more than 99%, with SEQ ID NO: 3657, wherein the CDRs are as defined above. In one embodiment, the ISVD specifically binding to PD-1 comprises or consists of the amino acid sequence of SEQ ID NO: 3657.

In another embodiment, the ISVD specifically binding to human PD-1 may have a sequence identity of more than 90%, such as more than 95% or even more than 99%, with SEQ ID NO: 3658, wherein the CDRs are as defined above. In one embodiment, the ISVD specifically binding to PD-1 comprises or consists of the amino acid sequence of SEQ ID NO: 3658.

In another embodiment, the ISVD specifically binding to human PD-1 may have a sequence identity of more than 90%, such as more than 95% or even more than 99%, with SEQ ID NO: 3659, wherein the CDRs are as defined above. In one embodiment, the ISVD specifically binding to PD-1 comprises or consists of the amino acid sequence of SEQ ID NO: 3659.

In another embodiment, the ISVD specifically binding to human PD-1 may have a sequence identity of more than 90%, such as more than 95% or even more than 99%, with SEQ ID NO: 3660, wherein the CDRs are as defined above. In one embodiment, the ISVD specifically binding to PD-1 comprises or consists of the amino acid sequence of SEQ ID NO: 3660.

In another embodiment, the ISVD specifically binding to human PD-1 may have a sequence identity of more than 90%, such as more than 95% or even more than 99%, with SEQ ID NO: 3661, wherein the CDRs are as defined above. In one embodiment, the ISVD specifically binding to PD-1 comprises or consists of the amino acid sequence of SEQ ID NO: 3661.

In another embodiment, the ISVD specifically binding to human PD-1 may have a sequence identity of more than 90%, such as more than 95% or even more than 99%, with SEQ ID NO: 3662, wherein the CDRs are as defined above. In one embodiment, the ISVD specifically binding to PD-1 comprises or consists of the amino acid sequence of SEQ ID NO: 3662.

In another embodiment, when such an ISVD specifically binding to PD-1 has 3, 2 or 1 amino acid difference in at least one CDR relative to a corresponding reference CDR sequence (above), the ISVD has at least (essentially) the same binding affinity to human PD-1 compared to one of the VHHs with SEQ ID NOs: 2857-3662 (preferably 3395, 3376, 3447, 3446, 3643, 2859 or 3653), wherein the binding affinity is measured using the same method, such as e.g. SPR.

In another embodiment, when such an ISVD specifically binding to PD-1 has 3, 2 or 1 amino acid difference in at least one CDR relative to a corresponding reference CDR sequence (above), the ISVD has at least (essentially) the same binding affinity to cyno PD-1 compared to one of the VHHs with SEQ ID NOs: 2857-3662 (preferably 3395, 3376, 3447, 3446, 3643, 2859 or 3653), wherein the binding affinity is measured using the same method, such as e.g. SPR.

In another embodiment, when such an ISVD specifically binding to PD-1 has 3, 2 or 1 amino acid difference in at least one CDR relative to a corresponding reference CDR sequence (above), the ISVD still specifically binds to human PD-1 and cyno PD-1 with less than 10-fold difference in affinity (K_{D}).

In another embodiment, when such an ISVD specifically binding to PD-1 has 3, 2 or 1 amino acid difference in at least one CDR relative to a corresponding reference CDR sequence (above), the ISVD still specifically binds to human and cyno PD-1 and does not bind to other members of the CD28 receptor family.

In another embodiment, when such an ISVD specifically binding to PD-1 has 3, 2 or 1 amino acid difference in at least one CDR relative to a corresponding reference CDR sequence (above), the ISVD still specifically binds to human and cyno PD-1 and does not bind to CD28, CTLA-4, ICOS, and BTLA.

In another embodiment, when such an ISVD specifically binding to PD-1 has 3, 2 or 1 amino acid difference in at least one CDR relative to a corresponding reference CDR sequence (above), the ISVD blocks the interaction of human PD-1 with human PD-L1 with at least (essentially) the same potency compared to one of the VHHs with SEQ ID NOs: 2857-3662 (preferably 3395, 3376, 3447, 3446, 3643, 2859 or 3653), wherein the potency is measured, e.g., in FACS competition assay on CHO cells expressing human PD-1, with hPD-L1 as competitor.

In another embodiment, when such an ISVD specifically binding to PD-1 has 3, 2 or 1 amino acid difference in at least one CDR relative to a corresponding reference CDR sequence (above), the ISVD blocks the interaction of cyno PD-1 with cyno PD-L1 with at least (essentially) the same potency compared to one of the VHHs with SEQ ID NOs: 2857-3662 (preferably 3395, 3376, 3447, 3446, 3643, 2859 or 3653), wherein the potency is measured, e.g., in FACS competition assay on CHO cells expressing cyno PD-1, with hPD-L1 as competitor.

In another embodiment, when such an ISVD specifically binding to PD-1 has 3, 2 or 1 amino acid difference in at least one CDR relative to a corresponding reference CDR sequence (above), the ISVD blocks the interaction of hPD-1 with PD-L1 with (essentially) the same % inhibition or more compared to one of the VHHs with SEQ ID NOs: 2857-3662 (preferably 3395, 3376, 3447, 3446, 3643, 2859 or 3653), wherein the % inhibition is determined, e.g., in a FACS competition assay.

In another embodiment, when such an ISVD specifically binding to PD-1 has 3, 2 or 1 amino acid difference in at least one CDR relative to a corresponding reference CDR sequence (above), the ISVD has at least (essentially) the same potency for T-cell activation compared to one of the VHHs with SEQ

ID NOs: 2857-3662 (preferably 3395, 3376, 3447, 3446, 3643, 2859 or 3653), wherein the potency is monitored, e.g., via IFN-gamma production in an autologous Tetanus Toxoid recall assay (TT assay), e.g. the TT assay as described herein.

In another embodiment, when such an ISVD specifically binding to PD-1 has 3, 2 or 1 amino acid difference in at least one CDR relative to a corresponding reference CDR sequence (above), the ISVD has at least (essentially) the same efficacy for T-cell activation compared to one of the VHHs with SEQ ID NOs: 2857-3662 (preferably 3395, 3376, 3447, 3446, 3643, 2859 or 3653), wherein the efficacy is monitored, e.g., via IFN-gamma production in an autologous Tetanus Toxoid recall assay (TT assay), e.g. the TT assay as described herein

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same binding affinity to human PD-1 compared to one of the VHHs with SEQ ID NOs: 2857-3662 (preferably 3395, 3376, 3447, 3446, 3643, 2859 or 3653), wherein the binding affinity is measured using the same method, such as e.g. SPR.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same binding affinity to cyno PD-1 compared to one of the VHHs with SEQ ID NOs: 2857-3662 (preferably 3395, 3376, 3447, 3446, 3643, 2859 or 3653), wherein the binding affinity is measured using the same method, such as e.g. SPR.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human PD-1 and cyno PD-1 with less than 10-fold difference in affinity (K_{D}).

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human and cyno PD-1 and does not bind to other members of the CD28 receptor family.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human and cyno PD-1 and does not bind to CD28, CTLA-4, ICOS, and BTLA.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of human PD-1 with human PD-L1 with at least (essentially) the same potency compared to one of the VHHs with SEQ ID NOs: 2857-3662 (preferably 3395, 3376, 3447, 3446, 3643, 2859 or 3653), wherein the potency is measured, e.g., in FACS competition assay on CHO cells expressing human PD-1, with hPD-L1 as competitor

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of cyno PD-1 with cyno PD-L1 with at least (essentially) the same potency compared to one of the VHHs with SEQ ID NOs: 2857-3662 (preferably 3395, 3376, 3447, 3446, 3643, 2859 or 3653), wherein the potency is measured, e.g., in FACS competition assay on CHO cells expressing cyno PD-1, with hPD-L1 as competitor

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of hPD-1 with (h) PD-L1 with (essentially) the same % inhibition or more compared to one of the VHHs with SEQ ID NOs: 2857-3662 (preferably VHHs with SEQ ID NO: 3395, 3376, 3447, 3446, 3643, 2859 or 3653), wherein the % inhibition is determined, e.g., in a FACS competition assay.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same potency for T-cell activation compared to one of the VHHs with SEQ ID NOs: 2857-3662 (preferably VHHs with SEQ ID NO: 3395, 3376, 3447, 3446, 3643, 2859 or 3653), wherein the potency is monitored via, e.g., IFN-gamma production in an autologous Tetanus Toxoid recall assay (TT assay), e.g. the TT assay as described herein.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same efficacy for T-cell activation compared to one of the VHHs with SEQ ID NOs: 2857-3662 (preferably VHHs with SEQ ID NO: 3395, 3376, 3447, 3446, 3643, 2859 or 3653), wherein the efficacy is monitored, e.g., via IFN-gamma production in an autologous Tetanus Toxoid recall assay (TT assay), e.g. the TT assay as described herein.

In another embodiment, when the ISVD specifically binding to human PD-1 has a sequence identity of more than 90%, such as more than 95% or more than 99%, with one of the VHHs with SEQ ID NOs: 2857-3662, the ISVD has at least (essentially) the same binding affinity to human PD-1 compared to one of the VHHs with SEQ ID NOs: 2857-3662 (preferably VHHs with SEQ ID NO: 3395, 3376, 3447, 3446, 3643, 2859 or 3653), wherein the binding affinity is measured using the same method, such as e.g. SPR.

In another embodiment, when the ISVD specifically binding to human PD-1 has a sequence identity of more than 90%, such as more than 95% or more than 99%, with one of the VHHs with SEQ ID NOS: 2857-3662, the ISVD has at least (essentially) the same binding affinity to cyno PD-1 compared to one of the VHHs with SEQ ID NOs: 2857-3662 (preferably VHHs with SEQ ID NO: 3395, 3376, 3447, 3446, 3643, 2859 or 3653), wherein the binding affinity is measured using the same method, such as e.g. SPR.

In another embodiment, when the ISVD specifically binding to human PD-1 has a sequence identity of more than 90%, such as more than 95% or more than 99%, with one of the VHHs with SEQ ID NOs: 2857-3662, the ISVD still specifically binds to human PD-1 and cyno PD-1 with less than 10-fold difference in affinity (K_{D}).

In another embodiment, when the ISVD specifically binding to human PD-1 has a sequence identity of more than 90%, such as more than 95% or more than 99%, with one of the VHHs with SEQ ID NOS: 2857-3662, the ISVD still specifically binds to human and cyno PD-1 and does not bind to other members of the CD28 receptor family.

In another embodiment, when the ISVD specifically binding to human PD-1 has a sequence identity of more than 90%, such as more than 95% or more than 99%, with one of the VHHs with SEQ ID NOs: 2857-3662, the ISVD still specifically binds to human and cyno PD-1 and does not bind to CD28, CTLA4, ICOS, and BTLA.

In another embodiment, when the ISVD specifically binding to human PD-1 has a sequence identity of more than 90%, such as more than 95% or more than 99%, with one of the VHHs with SEQ ID NOs: 2857-3662, the ISVD blocks the interaction of human PD-1 with human PD-L1 with at least (essentially) the same potency compared to one of the VHHs with SEQ ID NOs: 2857-3662 (preferably VHHs with SEQ ID NO: 3395, 3376, 3447, 3446, 3643, 2859 or 3653), wherein the potency is measured in wherein the potency is measured, e.g., in FACS competition assay on CHO cells expressing human PD-1, with hPD-L1 as competitor.

In another embodiment, when the ISVD specifically binding to human PD-1 has a sequence identity of more than 90%, such as more than 95% or more than 99%, with one of the VHHs with SEQ ID NOS: 2857-3662, the ISVD blocks the interaction of cyno PD-1 with cyno PD-L1 with at least (essentially) the same potency compared to one of the VHHs with SEQ ID NOs: 2857-3662 (preferably VHHs with SEQ ID NO: 3395, 3376, 3447, 3446, 3643, 2859 or 3653), wherein the potency is measured, e.g., in FACS competition assay on CHO cells expressing cyno PD-1, with hPD-L1 as competitor.

In another embodiment, when the ISVD specifically binding to human PD-1 has a sequence identity of more than 90%, such as more than 95% or more than 99%, with one of the VHHs with SEQ ID NOS: 2857-3662, the ISVD blocks the interaction of hPD-1 with (h) PD-L1 with (essentially) the same % inhibition or more compared to one of the VHHs with SEQ ID NOs: 2857-3662 (preferably VHHs with SEQ ID NO: 3395, 3376, 3447, 3446, 3643, 2859 or 3653), wherein the % inhibition is determined, e.g., in a FACS competition assay.

In another embodiment, when the ISVD specifically binding to human PD-1 has a sequence identity of more than 90%, such as more than 95% or more than 99%, with one of the VHHs with SEQ ID NOS: 2857-3662, the ISVD has at least (essentially) half of the potency, preferably at least (essentially) the same potency for T-cell activation compared to one of the VHHs with SEQ ID NOs: 2857-3662 (preferably VHHs with SEQ ID NO: 3395, 3376, 3447, 3446, 3643, 2859 or 3653), wherein the potency is monitored, e.g., via IFN-gamma production in an autologous Tetanus Toxoid recall assay (TT assay), e.g. the TT assay as described herein.

In another embodiment, when the ISVD specifically binding to human PD-1 has a sequence identity of more than 90%, such as more than 95% or more than 99%, with one of the VHHs with SEQ ID NOs: 2857-3662, the ISVD has at least 75% of the efficacy, preferably at least 80% or 90% of the efficacy, more preferably at least (essentially) the same efficacy T-cell activation compared to one of the VHHs with SEQ ID NOs: 2857-3662 (preferably VHHs with SEQ ID NO: 3395, 3376, 3447, 3446, 3643, 2859 or 3653), wherein the efficacy is monitored, e.g., via IFN-gamma production in an autologous Tetanus Toxoid recall assay (TT assay), e.g. the TT assay as described herein.

In some embodiments, the immunoglobulin single variable domains of the present technology specifically bind to human PD-1 with a dissociation constant (K_{D}) of 5×10⁻⁹ to 10⁻¹² moles/litre or less, and preferably 10⁻⁹ to 10⁻¹² moles/litre or less and more preferably 10⁻⁹ to 5×10⁻¹¹ moles/litre, such as 7.5×10⁻¹⁰ moles/litre, as determined by Surface Plasmon Resonance, e.g., measured at 25° C.

In some embodiments, the immunoglobulin single variable domains of the present technology specifically bind to human PD-1 with a kₒₙ-rate of between 10⁴ M⁻¹s⁻¹ to about 10⁷ M⁻¹s⁻¹, preferably between 5×10⁴ M⁻¹s⁻¹ and 10⁷ M⁻¹s⁻¹, more preferably between 5×10⁴ M⁻¹s⁻¹ and 5×10⁶ M⁻¹s⁻¹, such as between 5×10⁴ M⁻¹s⁻¹ and 1×10⁵ M⁻¹s⁻¹, such as 8.8×10⁴ M⁻¹s⁻¹, as determined by Surface Plasmon Resonance, e.g., measured at 25° C.

In some embodiments, the immunoglobulin single variable domains of the present technology specifically bind to human PD-1 with a k_{off} rate between 10⁻³ s⁻¹ (t1/2=0.69 s) and 10⁻⁶ s⁻¹ (providing a near irreversible complex with a t1/2 of multiple days), preferably between 10⁻³ s⁻¹ and 5×10⁻⁶ s⁻¹, more preferably between 10⁻⁴ s⁻¹ and 5×10⁻⁶ s⁻¹, such as between 10⁻⁴ s⁻¹ and 10⁻⁵ s⁻¹, such as 6.6×10⁻⁵ s⁻¹, as determined by Surface Plasmon Resonance, e.g., measured at 25° C.

In some embodiments, the immunoglobulin single variable domains of the present technology specifically bind to cyno PD-1 with a dissociation constant (K_{D}) of 1×10⁻⁸ to 10⁻¹² moles/litre or less, and preferably 10⁻⁸ to 10⁻¹¹ moles/litre or less and more preferably 1×10⁻⁸ to 10⁻¹⁰ moles/litre, such as 3.1×10⁻⁹ moles/litre, as determined by Surface Plasmon Resonance, e.g., measured at 25° C.

In some embodiments, the immunoglobulin single variable domains of the present technology specifically bind to cyno PD-1 with a kₒₙ-rate of between 10⁴ M⁻¹s⁻¹ to about 10⁷ M⁻¹s⁻¹, preferably between 10⁴ M⁻¹s⁻¹ and 10⁶ M⁻¹s⁻¹, more preferably between 10⁴ M⁻¹s⁻¹ and 5×10⁵ M⁻¹s⁻¹, such as between 10⁴ M⁻¹s⁻¹ and 10⁵ M⁻¹s⁻¹, such as 4.9×10⁴ M⁻¹s⁻¹, as determined by Surface Plasmon Resonance, e.g., measured at 25° C.

In some embodiments, the immunoglobulin single variable domains of the present technology specifically bind to cyno PD-1 with a k_{off} rate between 10⁻³ s⁻¹ (t1/2=0.69 s) and 10⁻⁸ s⁻¹ (providing a near irreversible complex with a t1/2 of multiple days), preferably between 10⁻³ s⁻¹ and 10⁻⁷ s⁻¹, more preferably between 5×104 s⁻¹ and 10⁻⁶ s⁻¹, such as between 5×10⁻⁴ s⁻¹ and 105 s⁻¹, such as 1.5×10⁻⁴ s⁻¹, as determined by Surface Plasmon Resonance, e.g., measured at 25° C.

In some embodiments, the immunoglobulin single variable domains of the present technology specifically bind to human PD-1 and cyno PD-1 with less than 10-fold difference in affinity (KD).

In some embodiments, the immunoglobulin single variable domains of the present technology specifically bind to human and cyno PD-1 and do not bind to other members of the CD28 receptor family.

In some embodiments, the immunoglobulin single variable domains of the present technology specifically bind to human and cyno PD-1 and do not bind to CD28, CTLA-4, ICOS, and BTLA. In the present technology, the binding of the ISVDs and polypeptides comprising the ISVD to PD-1 can also be measured in assays in which PD-1 is exposed on a cell surface (such as e.g. CHO Flp-In human PD-1 cells, CHO Flp-In cyno PD-1 cells). The Invitrogen™ Flp-In™ System allows integration and expression of the gene of interest (e.g. encoding human, cyno or mouse PD-1) in mammalian cells (e.g. CHO cells) at a specific genomic location. The Flp-In System involves introduction of a Flp Recombination Target (FRT) site into the genome of the mammalian cell line of choice. An expression vector containing the gene of interest is then integrated into the genome via Flp recombinasemediated DNA recombination at the FRT site (O'Gorman et al., 1991, Recombinase-Mediated Gene Activation and Site-Specific Integration in Mammalian Cells, Science 251, 1351-1355).

A preferred assay for measuring binding of the ISVD (or polypeptide comprising the ISVD) to PD-1 exposed on a cell surface is a FACS assay, such as e.g. the FACS assay as described in the examples, wherein binding of the ISVDs or polypeptides to PD-1 expressed on CHO is determined.

In some embodiments, the immunoglobulin single variable domains of the present technology specifically bind PD-1 and block the interaction of PD-1 with PD-L1. In some embodiments, the immunoglobulin single variable domains of the present technology specifically bind hPD-1 and block the interaction of hPD-1 with hPD-L1. In some embodiments, the immunoglobulin single variable domains of the present technology specifically bind cyno PD-1 and block the interaction of cyno PD-1 with cyno PD-L1.

In some embodiments, the immunoglobulin single variable domains of the present technology show a highly potency and/or efficacy (as a single variable domain) for binding PD-1 in competition with PD-L1. In some embodiments, the immunoglobulin single variable domains of the present technology can functionally block PD-1. More specifically, the ISVD can block the interaction between PD-1 and PD-L1. In some embodiments, the immunoglobulin single variable domains of the present technology antagonize an activity of PD-1.

In some embodiments, the immunoglobulin single variable domains of the present technology specifically bind to human PD-1 and block the interaction of human PD-1 with human PD-L1. For example, in FACS competition assay on CHO cells expressing human PD-1, with hPD-L1 as competitor, the immunoglobulin single variable domains of the present technology may have EC50 values of 5×10⁻⁸ M or lower, more preferably of 10⁻⁸ M or lower, or even of 10⁻⁹ M or lower. For example, in such FACS competition assay, the immunoglobulin single variable domains of the present technology may have EC50 values between 10⁻¹¹ M and 10⁻⁸ M, such as between 10⁻¹⁰ M and 5×10⁻⁹ M, between 10⁻¹⁰ M and 10⁻⁸ M or between 10⁻⁹ M and 10⁻⁸ M, such as 5.94×10⁻⁹ M.

In some embodiments, the immunoglobulin single variable domains of the present technology specifically bind to cyno PD-1 and block the interaction of cyno PD-1 with cyno or human PD-L1. For example, in a FACS competition assay on CHO cells expressing cyno PD-1, with human PD-L1 as competitor, the immunoglobulin single variable domains of the present technology may have EC50 values of 10⁻⁸ M or lower, more preferably of 5×10⁻⁹ M or lower, or even of 10⁻⁹ M or lower. For example, in such FACS competition assay, the immunoglobulin single variable domains of the present technology may have EC50 values between 10⁻¹¹ M and 10⁻⁸ M, such as between 10⁻¹⁰ M and 5×10⁻⁹ M, between 10⁻¹⁰ M and 10⁻⁸ M or between 10⁻⁹ M and 10⁻⁸ M, such as 4.7×10⁻⁹ M.

In some embodiments, the immunoglobulin single variable domains of the present technology block the binding of human PD-1 to human PD-L1 by at least 50%, such as at least 60%, 70%, 80%, 90%, 95% or even more, as determined by ligand competition, AlphaScreen, or competitive binding assays (such as competition ELISA or competition FACS).

In some embodiments, the immunoglobulin single variable domains of the present technology block the binding of cyno PD-1 to cyno PD-L1 by at least 50%, such as at least 60%, 70%, 80%, 90%, 95% or even more, as determined by ligand competition, AlphaScreen, or competitive binding assays (such as competition ELISA or competition FACS).

In some embodiments, the immunoglobulin single variable domains of the present technology show a highly potency for T cell activation as a single variable domain. T cell activation refers to a process in which mature T cells can express antigen-specific T cell receptors on their surface to recognize their cognate antigens and respond by entering the cell cycle, secreting cytokines or lytic enzymes, and initiating the cell-based functions of the immune system.

T-cell activation as used herein refers to one or more cellular response(s) of a T cell, e.g., a cytotoxic T cell, such as selected from: proliferation, differentiation, cytokine secretion, cytotoxic effector molecule release, cytotoxic activity, expression of activation markers, and redirected target cell lysis. The polypeptides of the invention are capable of inducing one or more of such responses. Suitable assays to measure T cell activation are known in the art, for instance as described in WO 99/54440 or by Schlereth et al. 2005 (Cancer Immunol. Immunother. 20:1-12), or as exemplified in the examples or below. For example, T cell activation can be measured by assessing (1) proliferation, (2) up regulation of activation markers (e.g., IL2RA/CD25) and/or (3) production of effector cytokines (IFNgamma, TNF-α).

In some embodiments, T cell activation by the ISVDs and polypeptides comprising the same can be monitored by upregulation of CD69, CD25 and various cell adhesion molecules, de novo expression and/or release of cytokines (e.g., IFN-γ, TNF-α, IL-6, IL-2, IL-4 and IL-10), upregulation of granzyme and perforin expression, and/or cell proliferation, membrane blebbing, activation of procaspases 3 and/or 7, fragmentation of nuclear DNA and/or cleavage of caspase substrate poly (ADPribose) polymerase.

In some embodiments, T cell activation is monitored by measurement of IFN-gamma release.

In some embodiments, T cell activation is measured by determining the expression of a number of molecules, namely CD25, CD69, CD71, and HLA-DR, also termed “activation antigens”, by activated proliferating lymphocytes on their surface.′ Such activation antigens are easily evidenced by immunofluorescence assay and flow cytometric analysis using monoclonal antibodies (mAbs) as specific reagents. By two- and three colour immunofluorescent staining it is also possible to identify, within bulk lymphocyte cultures, the T-cell subpopulations involved in the activation process.

In some embodiments, T cell activation can be measured by DNA synthesis assays. DNA synthesis assays use radioactively labelled 3H-thymine to detect DNA within cells. Newly proliferated cells incorporate the label, providing extremely sensitive and accurate detection of cell lines (see e.g., Madhavan 2007, J. Stem Cells Regen. Med. 3:12).

In some embodiments, T cell activation can be measured in a tetanus Toxoid recall assay (TT assay). The assay makes use of Lymphocytes that have previously been exposed to an antigen and that will show an enhanced response upon subsequent re-exposure. Primary lymphocytes from individuals vaccinated against tetanus are used to initiate an in vitro recall antigenic response. Stimulation of such lymphocytes with tetanus toxoid provides a useful assay method to assess the effects of candidate therapeutic molecules, such as checkpoint inhibitors, and as a model for analogous responses to tumor antigens.

Lymphocytes are antigenically stimulated following culture with loaded antigen presenting cells (APCs). Antigen stimulated lymphocytes are cultured in the presence of checkpoint inhibitors to modulate activation response. Endpoints that are assessed in this assay include lymphocyte proliferation, activation markers, and cytokine production (Ticha et al. 2021, A cell-based in vitro assay for testing of immunological integrity of Tetanus toxoid vaccine antigen, Npj Vaccines 6:1-11). In some embodiments, IFN-gamma production is measured following activation of the T lymphocytes.

In some embodiments, the immunoglobulin single variable domains of the present technology have a potency (EC50 values) in a TT assay (e.g. as described in the Examples), monitored via IFN-gamma production, of 10⁷ M or lower, more preferably of 5×10⁻⁸ M or lower, or even of 10⁻⁸ M or lower. For example, in such TT assay, the immunoglobulin single variable domains of the present technology may have a potency (EC50 values) between 10⁻¹¹ M and 10⁻⁷ M, such as between 10⁻¹⁰ M and 10⁻⁸ M, between 10⁻¹⁰ M and 10⁻⁷ M or between 10⁻⁸ M and 10⁻⁷ M, such as 2.69×10⁻⁸ M, or 3.37×10⁻⁸ M.

In some embodiments, a polypeptide comprising at least two immunoglobulin single variable domains of the present technology has a potency (EC50 values) in a TT assay (e.g. as described in the Examples), monitored via IFN-gamma production, of 10⁻⁸ M or lower, more preferably of 10⁻⁹ M or lower, or even of 5.10-10 M or lower. For example, in such TT assay, the immunoglobulin single variable domains of the present technology may have a potency (EC50 values) between 10⁻¹¹ M and 10⁻⁸ M, such as between 10⁻¹⁰ M and 10⁻⁸ M, between 10⁻¹⁰ M and 10⁻⁹ M or between 10⁻¹¹ M and 10⁻⁹ M, such as 8.9×10⁻¹⁰ M or 3.8×10⁻¹⁰ M.

In some embodiments, the immunoglobulin single variable domains of the present technology have an efficacy in a TT assay (e.g., as described in the Examples), monitored via IFN-gamma production, of at least 50%, such as at least 60%, 70%, 80%, 90%, 95%.

The T Cell Activation Bioassay (NFAT) is a bioluminescent cell-based assay that consists of a genetically engineered Jurkat T cell line that expresses a luciferase reporter driven by an NFAT-response element (NFAT-RE). When the TCR/CD3 Effector Cells (NFAT) are engaged, the TCR transduces intracellular signals resulting in NFAT-RE-mediated luminescence. The bioluminescent signal is detected and quantified using Bio-Glo™ Luciferase Assay System and a standard luminometer, such as the GloMax® Discover System.

In some embodiments, the immunoglobulin single variable domains of the present technology have a potency (IC50 values) in a NFAT assay (e.g., as described in the Examples) of 10⁻⁷ M or lower, of 10⁻⁸ M or lower, more preferably of 5×10⁻⁸ M or lower, or even of 10⁻⁹ M or lower, or even 5×10⁻¹⁰ or lower. For example, in such NFAT assay, the immunoglobulin single variable domains of the present technology may have a potency (IC50 values) between 10⁻¹⁰ M and 10⁻⁷ M, such as between 10⁻¹⁰ M and 10⁻⁸ M or between 10⁻¹⁰ M and 10⁻⁹ M, such as 0.9×10⁻⁹ M.

In some embodiments, the immunoglobulin single variable domains of the present technology block the binding of human PD-L1 to human PD-1 by at least 50%, such as at least 60%, 70%, 80%, 90%, 95% or even more, as determined in a NFAT assay.

In some embodiments, the immunoglobulin single variable domains of the present technology block the binding of human or cyno PD-L1 to cyno PD-1 by at least 50%, such as at least 60%, 70%, 80%, 90%, 95% or even more, as determined in a NFAT assay.

In another embodiment, when the ISVD specifically binding to human PD-1 has a sequence identity of more than 90%, such as more than 95% or more than 99%, with one of the VHHs with SEQ ID NOS: 2857-3662, the ISVD blocks the interaction of hPD-1 with (h) PD-L1 with (essentially) the same % inhibition or more compared to one of the VHHs with SEQ ID NOs: 3395, 3376, 3447, 3446, 3643, 2859 or 3653, wherein the % inhibition is determined in a NFAT assay.

### 5.2.4 Embodiment 4, Family A0294040C12

Specific examples of ISVDs specifically binding to PD-1 are ISVDs that comprises 4 framework regions (FR1 to FR4, respectively) and three complementarity determining regions (CDR1 to CDR3, respectively), wherein:
CDR1 (AbM definition) has an amino acid sequence selected from:
   a) the amino acid sequence of GFTLX₁X₂X₃X₄X₅X₆ (SEQ ID NO: 4696), wherein X₁ is selected from Y, F, T, Q, A, S, R, P, D, G, L, E, K, H, or D, X₂ is selected from D, R, T, I, Q, F, H, S, L, V, K, E, P, G, A, or Y, X₃ is selected from H, G, Y, R, D, E, V, P, Q, S, A, K, I, F, T, or L, X₄ is selected from A, L, S, D, R, K, H, Q, T, V, Y, P, I, F, or G, X₅ is selected from V and F, and X₆ is selected from G, Q, E, H, K, P, Y, D, I, T, A, S, R, V, or C;
   b) amino acid sequences that have at least 80% amino acid identity with the amino acid sequence of GFTLX₁X₂X₃X₄X₅X₆ (SEQ ID NO: 4696), wherein X₁ is selected from Y, F, T, Q, A, S, R, P, D, G, L, E, K, H, or D, X₂ is selected from D, R, T, I, Q, F, H, S, L, V, K, E, P, G, A, or Y, X₃ is selected from H, G, Y, R, D, E, V, P, Q, S, A, K, I, F, T, or L, X₄ is selected from A, L, S, D, R, K, H, Q, T, V, Y, P, I, F, or G, X₅ is selected from V and F, and X₆ is selected from G, Q, E, H, K, P, Y, D, I, T, A, S, R, V, or C;
   c) amino acid sequences that have 3, 2, or 1 amino acid difference with the amino acid sequences of GFTLX₁X₂X₃X₄X₅X₆ (SEQ ID NO: 4696), wherein X₁ is selected from Y, F, T, Q, A, S, R, P, D, G, L, E, K, H, or D, X₂ is selected from D, R, T, I, Q, F, H, S, L, V, K, E, P, G, A, or Y, X₃ is selected from H, G, Y, R, D, E, V, P, Q, S, A, K, I, F, T, or L, X₄ is selected from A, L, S, D, R, K, H, Q, T, V, Y, P, I, F, or G, X₅ is selected from V and F, and X₆ is selected from G, Q, E, H, K, P, Y, D, I, T, A, S, R, V, or C;
and
CDR2 (AbM definition) has an amino acid sequence selected from:
   a) the amino acid sequence of X₁X₂SSSGX₃HEX₄ (SEQ ID NO: 4697), wherein X₁ is selected from V, E, or D, X₂ is selected from I, P, T, S, R, G, H, or K, X₃ is selected from A, R, H, E, K, T, or P, and X₄ is selected from W, F, E, P, D, H, Y, L, R, V, K, G, S, A, or l;
   b) amino acid sequences that have at least 80% amino acid identity with the amino acid sequence of X₁X₂SSSGX₃HEX₄ (SEQ ID NO: 4697), wherein X₁ is selected from V, E, or D, X₂ is selected from I, P, T, S, R, G, H, or K, X₃ is selected from A, R, H, E, K, T, or P, and X₄ is selected from W, F, E, P, D, H, Y, L, R, V, K, G, S, A, or l;
   c) amino acid sequences that have 3, 2, or 1 amino acid difference with the amino acid sequence of X₁X₂SSSGX₃HEX₄ (SEQ ID NO: 4697), wherein X₁ is selected from V, E, or D, X₂ is selected from I, P, T, S, R, G, H, or K, X₃ is selected from A, R, H, E, K, T, or P, and X₄ is selected from W, F, E, P, D, H, Y, L, R, V, K, G, S, A, or l;
CDR3 (AbM definition) has an amino acid sequence selected from:
   a) the amino acid sequence of GX₁X₂X₃YSX₄X₅X₆SEY (SEQ ID NO: 4698); wherein X₁ is selected from G or R, X₂ is selected from M, E, Q, F, V, G, A, I, D, Y, R, K, S, T, H, or P, X₃ is selected from W, L, T, S, P, F, V, I, R, L, Q, E, K, G, D, Y, H, or A, X₄ is selected from D, G, H, K, P, R, or A, X₅ is selected from D, A, V, S, E, I, H, Y, L, G, P, K, Q, R, T, or F, and X₆ is selected from G, F, S, H, Y, I, R, K, L, T, P, V, D, E, Q, or A;
   b) amino acid sequences that have at least 80% amino acid identity with the amino acid sequence of GX₁X₂X₃YSX₄X₅X₆SEY (SEQ ID NO: 4698); wherein X₁ is selected from G or R, X₂ is selected from M, E, Q, F, V, G, A, I, D, Y, R, K, S, T, H, or P, X₃ is selected from W, L, T, S, P, F, V, I, R, L, Q, E, K, G, D, Y, H, or A, X₄ is selected from D, G, H, K, P, R, or A, X₅ is selected from D, A, V, S, E, I, H, Y, L, G, P, K, Q, R, T, or F, and X₆ is selected from G, F, S, H, Y, I, R, K, L, T, P, V, D, E, Q, or A;
   c) amino acid sequences that have 3, 2, or 1 amino acid difference with the amino acid sequence of GX₁X₂X₃YSX₄X₅X₆SEY (SEQ ID NO: 4698); wherein X₁ is selected from G or R, X₂ is selected from M, E, Q, F, V, G, A, I, D, Y, R, K, S, T, H, or P, X₃ is selected from W, L, T, S, P, F, V, I, R, L, Q, E, K, G, D, Y, H, or A, X₄ is selected from D, G, H, K, P, R, or A, X₅ is selected from D, A, V, S, E, I, H, Y, L, G, P, K, Q, R, T, or F, and X₆ is selected from G, F, S, H, Y, I, R, K, L, T, P, V, D, E, Q, or A.

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NO: 4696, 4697 and 4698.

In some embodiments, the ISVD essentially consists of 4 framework regions (FR1 to FR4, respectively) and 3 complementarity determining regions (CDR1 to CDR3, respectively), in which:
CDR1 (AbM numbering) has an amino acid sequence selected from:
   a) the amino acid sequence of GFTLYDHAXG (SEQ ID NO: 4611), wherein X is selected from V or F;
   b) amino acid sequences that have at least 80% amino acid identity with the amino acid sequence of GFTLYDHAXG (SEQ ID NO: 4611), wherein X is selected from V or F;
   c) amino acid sequences that have 3, 2, or 1 amino acid difference with the amino acid sequences of GFTLYDHAXG (SEQ ID NO: 4611), wherein X is selected from V or F;
and
CDR2 (AbM numbering) has an amino acid sequence selected from:
   a) the amino acid sequence of VISSSGAHEW (SEQ ID NO: 4481);
   b) amino acid sequences that have at least 80% amino acid identity with the amino acid sequence of VISSSGAHEW (SEQ ID NO: 4481);
   c) amino acid sequences that have 3, 2, or 1 amino acid difference with the amino acid sequence of VISSSGAHEW (SEQ ID NO: 4481);
and
CDR3 (AbM numbering) has an amino acid sequence selected from:
   a) the amino acid sequence of GGMWYSDXGSEY (SEQ ID NO: 4612); wherein X is selected from D or E;
   b) amino acid sequences that have at least 80% amino acid identity with the amino acid sequence of GGMWYSDXGSEY (SEQ ID NO: 4612); wherein X is selected from D or E;
   c) amino acid sequences that have 3, 2, or 1 amino acid difference with the amino acid sequence of GGMWYSDXGSEY (SEQ ID NO: 4612); wherein X is selected from D or E.

Preferably, the CDR sequences have at least 90% amino acid sequence identity, more preferably at least 95% amino acid sequence identity, such as 99% amino acid sequence identity or more, or even essentially 100% amino acid sequence identity with the CDR sequences of SEQ ID NO: 4611, SEQ ID NO: 4481 or SEQ ID NO: 4612.

In some embodiments, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of GFTLYDHAVG (SEQ ID NO: 4412);
   b) amino acid sequences that have at least 80% amino acid identity with the amino acid sequence of SEQ ID NO: GFTLYDHAVG (SEQ ID NO: 4412); and
   c) amino acid sequences that have 3, 2, or 1 amino acid difference with the amino acid sequences of SEQ ID NO: GFTLYDHAVG (SEQ ID NO: 4412);
and
CDR2 (AbM definition) has an amino acid sequence selected from:
   a) the amino acid sequence of VISSSGAHEW (SEQ ID NO: 4481);
   b) amino acid sequences that have at least 80% amino acid identity with the amino acid sequence VISSSGAHEW (SEQ ID NO: 4481);
   c) amino acid sequences that have 3, 2, or 1 amino acid difference with the amino acid sequence of VISSSGAHEW (SEQ ID NO: 4481);
and
CDR3 (AbM definition) has an amino acid sequence selected from:
   a) the amino acid sequence of GGMWYSDDGSEY (SEQ ID NOs: 4560);
   b) amino acid sequences that have at least 80% amino acid identity with the amino acid sequence of SEQ ID NO: GGMWYSDDGSEY (SEQ ID NOs: 4560);
   c) amino acid sequences that have 3, 2, or 1 amino acid difference with the amino acid sequence of SEQ ID NO: GGMWYSDDGSEY (SEQ ID NOs: 4560).

In some embodiments, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of GFTLYDHAVG (SEQ ID NO: 4412);
   b) amino acid sequences that have at least 80% amino acid identity with the amino acid sequence of SEQ ID NO: GFTLYDHAVG (SEQ ID NO: 4412); and
   c) amino acid sequences that have 3, 2, or 1 amino acid difference with the amino acid sequences of SEQ ID NO: GFTLYDHAVG (SEQ ID NO: 4412);
and
CDR2 (AbM definition) has an amino acid sequence selected from:
   a) the amino acid sequence of VISSSGAHEW (SEQ ID NO: 4481);
   b) amino acid sequences that have at least 80% amino acid identity with the amino acid sequence VISSSGAHEW (SEQ ID NO: 4481);
   c) amino acid sequences that have 3, 2, or 1 amino acid difference with the amino acid sequence of VISSSGAHEW (SEQ ID NO: 4481);
and
CDR3 (AbM definition) has an amino acid sequence selected from:
   a) the amino acid sequence of GGMWYSDEGSEY (SEQ ID NOs: 4572);
   b) amino acid sequences that have at least 80% amino acid identity with the amino acid sequence of SEQ ID NO: GGMWYSDEGSEY (SEQ ID NOs: 4572);
   c) amino acid sequences that have 3, 2, or 1 amino acid difference with the amino acid sequence of SEQ ID NO: GGMWYSDEGSEY (SEQ ID NOs: 4572).

In some embodiments, the ISVD essentially consists of 4 framework regions (FR1 to FR4, respectively) and 3 complementarity determining regions (CDR1 to CDR3 respectively), in which:
CDR1 (AbM definition) has an amino acid sequence selected from:
   a) the amino acid sequence of GFTLYDHAFG (SEQ ID NO: 4408);
   b) amino acid sequences that have at least 80% amino acid identity with the amino acid sequence of SEQ ID NO: GFTLYDHAFG (SEQ ID NO: 4408); and
   c) amino acid sequences that have 3, 2, or 1 amino acid difference with the amino acid sequences of SEQ ID NO: GFTLYDHAFG (SEQ ID NO: 4408);
and
CDR2 (AbM definition) has an amino acid sequence selected from:
   a) the amino acid sequence of VISSSGAHEW (SEQ ID NO: 4481);
   b) amino acid sequences that have at least 80% amino acid identity with the amino acid sequence VISSSGAHEW (SEQ ID NO: 4481);
   c) amino acid sequences that have 3, 2, or 1 amino acid difference with the amino acid sequence of VISSSGAHEW (SEQ ID NO: 4481);
and
CDR3 (AbM definition) has an amino acid sequence selected from:
   a) the amino acid sequence of GGMWYSDEGSEY (SEQ ID NOs: 4572);
   b) amino acid sequences that have at least 80% amino acid identity with the amino acid sequence of SEQ ID NO: GGMWYSDEGSEY (SEQ ID NOs: 4572);
   c) amino acid sequences that have 3, 2, or 1 amino acid difference with the amino acid sequence of SEQ ID NO: GGMWYSDEGSEY (SEQ ID NOs: 4572).

In one embodiment, the ISVD comprises CDRs (AbM definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 4049-4378.

In one embodiment, the ISVD comprises CDRs (AbM definition) with an amino acid sequences that has at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 4056, 4094, 4183, or 4210.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4524.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4528.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4470, and a CDR3 (AbM numbering) that is the amino acid sequence of SEQ ID NO: 4528.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4527.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4470, and a CDR3 (AbM numbering) that is the amino acid sequence of SEQ ID NO: 4527.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4524.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4596.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4528.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4470, and a CDR3 (AbM numbering) that is the amino acid sequence of SEQ ID NO: 4524.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4524.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4470, and a CDR3 (AbM numbering) that is the amino acid sequence of SEQ ID NO: 4531.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4531.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4470, and a CDR3 (AbM numbering) that is the amino acid sequence of SEQ ID NO: 4526.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4526.

In one embodiment, the ISVD comprises a CDR1 (AbM numbering) that is the amino acid sequence of SEQ ID NO: 4417, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4527.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4531.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4555.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4533.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4532.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4528.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4527.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4605.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4604.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4601.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4603.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4584.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4582.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4555.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4533.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4532.

In one embodiment, the ISVD comprises a CDR1 (AbM numbering) that is the amino acid sequence of SEQ ID NO: 4411, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4593.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4470, and a CDR3 (AbM numbering) that is the amino acid sequence of SEQ ID NO: 4593.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4582.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4529.

In one embodiment, the ISVD comprises a CDR1 (AbM numbering) that is the amino acid sequence of SEQ ID NO: 4429, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4433, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4559.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4547.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4561.

In one embodiment, the ISVD comprises a CDR1 (AbM numbering) that is the amino acid sequence of SEQ ID NO: 4423, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4432, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4413, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4571.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4562.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4567.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4563.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4564.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4569.

In one embodiment, the ISVD comprises a CDR1 (AbM numbering) that is the amino acid sequence of SEQ ID NO: 4428, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4426, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4431, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4415, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4607.

In one embodiment, the ISVD comprises a CDR1 (AbM numbering) that is the amino acid sequence of SEQ ID NO: 4416, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4422, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4410, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4434, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4435, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4436, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4430, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4414, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4420, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4427, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4424, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4425, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4409, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4419, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4418, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4421, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4584.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4597.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4490, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4493, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4492, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4492, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4491, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4491, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4466, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4467, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4467, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4489, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4466, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4467, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4490, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4587.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4489, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4467, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4468, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4490, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4464, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4463, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4464, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4464, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4463, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4493, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4493, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4492, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4491, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4492, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4489, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4588.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4589.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4466, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4590.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4531.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4488, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4601.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4470, and a CDR3 (AbM numbering) that is the amino acid sequence of SEQ ID NO: 4582.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4565.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4596.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4593.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4595.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4584.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4470, and a CDR3 (AbM numbering) that is the amino acid sequence of SEQ ID NO: 4584.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4582.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4555.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4470, and a CDR3 (AbM numbering) that is the amino acid sequence of SEQ ID NO: 4555.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4570.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4470, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4533.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4532.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4529.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4604.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4605.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4526.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4557.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4526.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4485, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4597.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4484, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4485, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4485, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4488, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4488, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4486, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4486, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4481, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4485, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4486, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4488, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4486, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4487, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4481, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4603.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4558.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4568.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4566.

In one embodiment, the ISVD comprises a CDR1 (AbM numbering) that is the amino acid sequence of SEQ ID NO: 4456, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4458, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4451, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4455, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4448, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4392, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4401, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4446, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4450, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4457, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4398, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4388, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4400, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4399, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4397, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4389, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4453, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4393, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4396, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4391, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4407, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4442, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4404, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4405, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4445, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4440, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4441, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4443, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4403, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4438, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4459, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4452, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4447, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4454, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4449, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4460, a CDR2 (AbM numbering) that is the amino acid sequence of SEQ ID NO: 4482, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4395, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4394, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4437, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4556.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4554.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4463, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4408, a CDR2 (AbM numbering) that is the amino acid sequence of SEQ ID NO: 4493, and a CDR3 (AbM numbering) that is the amino acid sequence of SEQ ID NO: 4572.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4465, and a CDR3 (AbM numbering) that is the amino acid sequence of SEQ ID NO: 4586.

In one embodiment, the ISVD comprises a CDR1 (AbM numbering) that is the amino acid sequence of SEQ ID NO: 4406, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4472, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4552.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4551.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4548.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4541.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4553.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4544.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4550.

In one embodiment, the ISVD comprises a CDR1 (AbM numbering) that is the amino acid sequence of SEQ ID NO: 4390, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4546.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4549.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4540.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4545.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4542.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4539.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4534.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4522.

In one embodiment, the ISVD comprises a CDR1 (AbM numbering) that is the amino acid sequence of SEQ ID NO: 4402, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4591.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4602.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4525.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4576.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4575.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4585.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4578.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4574.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4579.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4577.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4580.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4581.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4583.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4573.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4536.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4543.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4538.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4523.

In one embodiment, the ISVD comprises a CDR1 (AbM numbering) that is the amino acid sequence of SEQ ID NO: 4444, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4470, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4471, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4474, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4483, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4477, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4479, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4476, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4473, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4480, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4469, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4475, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4606.

In one embodiment, the ISVD comprises a CDR1 (AbM numbering) that is the amino acid sequence of SEQ ID NO: 4439, a CDR2 (AbM numbering) that is the amino acid sequence of SEQ ID NO: 4481, and a CDR3 (AbM numbering) that is the amino acid sequence of SEQ ID NO: 4560.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4598.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4537.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4599.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4600.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4530.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4594.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4535.

In one embodiment, the ISVD comprises a CDR1 (AbM numbering) that is the amino acid sequence of SEQ ID NO: 4412, a CDR2 (AbM numbering) that is the amino acid sequence of SEQ ID NO: 4481, and a CDR3 (AbM numbering) that is the amino acid sequence of SEQ ID NO: 4592.

In some embodiments, the ISVD has 80% amino acid sequence identity with one of the amino acid sequences of SEQ ID NOs: 4049-4378, in which for the purposes of determining the degree of amino acid identity, the amino acid residues that form the CDR sequences are disregarded; and in which:

preferably one or more of the amino acid residues at positions 11, 37, 44, 45, 47, 83, 84, 103, 104 and 108 according to the Kabat numbering are chosen from the Hallmark residues mentioned in Table 1.

In some embodiments, the ISVD is a humanized ISVD that is chosen from SEQ ID NOs: 4094-4055, or 4057-4378 or from the group consisting of amino acid sequences that have more than 80%, preferably more than 90%, more preferably more than 95%, such as 99% or more amino acid sequence identity with at least one of the amino acid sequences of SEQ ID NOs: 4094-4378.

In some embodiments, the amino acid sequence is chosen from the group consisting of SEQ ID NO: 4056 and SEQ ID NOs: 4094-4055, or 4057-4378.

In some embodiments, the amino acid sequence
i) has 80% amino acid sequence identity with one of the amino acid sequences of SEQ ID NOs: 4056, 4094, 4183, or 4210, in which for the purposes of determining the degree of amino acid identity, the amino acid residues that form the CDR sequences are disregarded;
and in which:
ii) preferably one or more of the amino acid residues at positions 11, 37, 44, 45, 47, 83, 84, 103, 104 and 108 according to the Kabat numbering are chosen from the Hallmark residues mentioned in Table 1.

In some embodiments, the ISVD essentially consists of a heavy chain variable domain sequence that is derived from a conventional four-chain antibody or that essentially consist of a heavy chain variable domain sequence that is derived from heavy chain antibody.

In some embodiments, the ISVD essentially consists of a VHH, a humanized VHH, a camelized VH, a domain antibody, a single domain antibody, or a dAb.

In some embodiments, the ISVD is a humanized ISVD that is chosen from SEQ ID NO: 4094, 4183, or 4210 or from the group consisting of amino acid sequences that have more than 80%, preferably more than 90%, more preferably more than 95%, such as 99% or more amino acid sequence identity with at least one of the amino acid sequences of SEQ ID NOs: 4056, 4094, 4183, or 4210.

In some embodiments, the amino acid sequence is chosen from the group consisting of SEQ ID NO: 4056 and SEQ ID NOs: 4094, 4183, or 4210.

In some embodiments, the ISVD specifically binds to human PD-1 with a dissociation constant (K_{D}) of 5×10⁻⁹ to 10⁻¹² moles/litre or less, and preferably 10⁻⁹ to 10⁻¹² moles/litre or less and more preferably 5×10⁻¹⁰ to 1×10⁻¹¹ moles/litre, such as 1.2×10⁻¹⁰ moles/litre, as determined by Surface Plasmon Resonance, e.g., measured at 25° C.

In some embodiments, the ISVD specifically binds to human PD-1 with a kₒₙ-rate of between 10⁴ M⁻¹ s⁻¹ to about 10⁷ M⁻¹s⁻¹, preferably between 10⁵ M⁻¹s⁻¹ and 10⁷ M⁻¹s⁻¹, more preferably between 10⁵ M⁻¹ s⁻¹ and 10⁶ M⁻¹s⁻¹, such as between 10⁵ M⁻¹s⁻¹ and 1×10⁶ M⁻¹s⁻¹, such as 3.7×10⁵ M⁻¹s⁻¹ or 3.0×10⁵ M⁻¹ s⁻¹ as determined by Surface Plasmon Resonance, e.g., measured at 25° C.

In some embodiments, the ISVD specifically binds to human PD-1 with a k_{off} rate between 10⁻³ s⁻¹ (t1/2=0.69 s) and 10⁻⁷ s⁻¹ (providing a near irreversible complex with a t1/2 of multiple days), preferably between 10⁻³ s⁻¹ and 5×10⁻⁶ s⁻¹, more preferably between 5×10⁻⁴ s⁻¹ and 5×10⁻⁶ s⁻¹, such as between 1×10⁻⁴ s⁻¹ and 10⁻⁵ s⁻¹, such as 3.5×10⁻⁵ s⁻¹, as determined by Surface Plasmon Resonance, e.g., measured at 25° C.

In some embodiments, the ISVD specifically binds to human and cyno PD-1 and does not bind to CD28, CTLA-4, ICOS, and BTLA or other members of the CD28 receptor family.

In some embodiments, the ISVD antagonizes an activity of PD-L1.

In some embodiments, the ISVD blocks the interaction of human PD-1 with human PD-L1 with a potency (IC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 10⁻⁹ M, between 10⁻¹⁰ M and 10⁻⁸ M or between 10⁻⁹ M and 10⁻⁸ M, such as 3.08×10⁻⁹ M, for example, as measured in a FACS competition assay on CHO cells expressing human PD-1 with hPD-L1 as a competitor.

In some embodiments, the ISVD blocks the interaction of cyno PD-1 with cyno PD-L1 with a potency (IC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 10⁻⁹ M, between 10⁻¹⁰ M and 10⁻⁸ M or between 10⁻⁹ M and 10⁻⁸ M, such as 3.57×10⁻⁹ M, for example, as measured in a as measured in a FACS competition assay on CHO cells expressing cyno PD-1 with hPD-L1 as a competitor.

In some embodiments, the ISVD blocks the binding of hPD-1 to hPD-L1 by at least 50%, such as at least 60%, 70%, 80%, 90%, 95%, 99% or even more, as determined by ligand competition, AlphaScreen, or competitive binding assays (such as competition ELISA or competition FACS).

In some embodiments, the ISVD induces T-cell activation with a potency (EC50 value) of 10⁻⁷ M or lower, more preferably of 5×10⁻⁸ M or lower, or even of 10⁻⁸ M or lower, such as between 10⁻¹¹ M and 10⁻⁷ M, such as between 10⁻¹⁰ M and 5×10⁻⁸ M, between 10⁻¹⁰ M and 10⁻⁸ M or between 10⁻⁸ M and 10 7 M, such as 5.68×10⁻⁸ M, 3.60×10⁻⁸ M or 5.8×10⁻⁸ M, as determined in a TT assay.

Specific examples of such ISVDs that specifically bind to PD-1 have one or more, or all, framework regions as indicated for A0294040C12 and sequence optimized variants thereof in Table A-5 and A10 (in addition to the CDRs as defined above). In one embodiment, the ISVD comprises or consists of the full amino acid sequence of A029400049 (SEQ ID NO: 4049), A029404631 (SEQ ID NO: 4050), A029404630 (SEQ ID NO: 4051), A029404629 (SEQ ID NO: 4052), A029404628 (SEQ ID NO: 4053), A029404627 (SEQ ID NO: 4054), A029404626 (SEQ ID NO: 4055), A029404654 (SEQ ID NO: 4057), A029404633 (SEQ ID NO: 4058), A029404026 (SEQ ID NO: 4059), A029404025 (SEQ ID NO: 4060), A029404024 (SEQ ID NO: 4061), A029404023 (SEQ ID NO: 4062), A029404022 (SEQ ID NO: 4063), A029404020 (SEQ ID NO: 4064), A029403857 (SEQ ID NO: 4065), A029404632 (SEQ ID NO: 4066), A029401893 (SEQ ID NO: 4067), A029404634 (SEQ ID NO: 4068), A029404651 (SEQ ID NO: 4069), A029404650 (SEQ ID NO: 4070), A029404649 (SEQ ID NO: 4071), A029404648 (SEQ ID NO: 4072), A029404647 (SEQ ID NO: 4073), A029404646 (SEQ ID NO: 4074), A029404645 (SEQ ID NO: 4075), A029404644 (SEQ ID NO: 4076), A029404643 (SEQ ID NO: 4077), A029404642 (SEQ ID NO: 4078), A029404641 (SEQ ID NO: 4079), A029404640 (SEQ ID NO: 4080), A029404639 (SEQ ID NO: 4081), A029404638 (SEQ ID NO: 4082), A029404637 (SEQ ID NO: 4083), A029404636 (SEQ ID NO: 4084), A029403855 (SEQ ID NO: 4085), A029404027 (SEQ ID NO: 4086), A029404028 (SEQ ID NO: 4087), A029404652 (SEQ ID NO: 4088), A029404635 (SEQ ID NO: 4089), A029403830 (SEQ ID NO: 4090), A029403783 (SEQ ID NO: 4091), A029403747 (SEQ ID NO: 4092), A029403746 (SEQ ID NO: 4093), A029403745 (SEQ ID NO: 4094), A029402104 (SEQ ID NO: 4095), A029402093 (SEQ ID NO: 4096), A029402073 (SEQ ID NO: 4097), A029403827 (SEQ ID NO: 4098), A029403785 (SEQ ID NO: 4099), A029403787 (SEQ ID NO: 4100), A029402053 (SEQ ID NO: 4101), A029402051 (SEQ ID NO: 4102), A029402050 (SEQ ID NO: 4103), A029402049 (SEQ ID NO: 4104), A029402048 (SEQ ID NO: 4105), A029402047 (SEQ ID NO: 4106), A029403784 (SEQ ID NO: 4107), A029403842 (SEQ ID NO: 4108), A029403782 (SEQ ID NO: 4109), A029403844 (SEQ ID NO: 4110), A029402066 (SEQ ID NO: 4111), A029403814 (SEQ ID NO: 4112), A029403813 (SEQ ID NO: 4113), A029403812 (SEQ ID NO: 4114), A029403810 (SEQ ID NO: 4115), A029403807 (SEQ ID NO: 4116), A029403806 (SEQ ID NO: 4117), A029403805 (SEQ ID NO: 4118), A029403803 (SEQ ID NO: 4119), A029403801 (SEQ ID NO: 4120), A029403798 (SEQ ID NO: 4121), A029403796 (SEQ ID NO: 4122), A029403793 (SEQ ID NO: 4123), A029403792 (SEQ ID NO: 4124), A029403791 (SEQ ID NO: 4125), A029403789 (SEQ ID NO: 4126), A029403788 (SEQ ID NO: 4127), A029404653 (SEQ ID NO: 4128), A029401895 (SEQ ID NO: 4129), A029404655 (SEQ ID NO: 4130), A029404845 (SEQ ID NO: 4131), A029404844 (SEQ ID NO: 4132), A029404843 (SEQ ID NO: 4133), A029404842 (SEQ ID NO: 4134), A029404841 (SEQ ID NO: 4135), A029404840 (SEQ ID NO: 4136), A029404847 (SEQ ID NO: 4137), A029404849 (SEQ ID NO: 4138), A029404850 (SEQ ID NO: 4139), A029404839 (SEQ ID NO: 4140), A029404835 (SEQ ID NO: 4141), A029404834 (SEQ ID NO: 4142), A029404833 (SEQ ID NO: 4143), A029404832 (SEQ ID NO: 4144), A029404830 (SEQ ID NO: 4145), A029404838 (SEQ ID NO: 4146), A029404837 (SEQ ID NO: 4147), A029404836 (SEQ ID NO: 4148), A029404846 (SEQ ID NO: 4149), A029404869 (SEQ ID NO: 4150), A029404867 (SEQ ID NO: 4151), A029404864 (SEQ ID NO: 4152), A029404863 (SEQ ID NO: 4153), A029404862 (SEQ ID NO: 4154), A029404861 (SEQ ID NO: 4155), A029404859 (SEQ ID NO: 4156), A029404858 (SEQ ID NO: 4157), A029404857 (SEQ ID NO: 4158), A029404856 (SEQ ID NO: 4159), A029404855 (SEQ ID NO: 4160), A029404854 (SEQ ID NO: 4161), A029404853 (SEQ ID NO: 4162), A029404852 (SEQ ID NO: 4163), A029404851 (SEQ ID NO: 4164), A029404829 (SEQ ID NO: 4165), A029404828 (SEQ ID NO: 4166), A029404848 (SEQ ID NO: 4167), A029404827 (SEQ ID NO: 4168), A029404656 (SEQ ID NO: 4169), A029404826 (SEQ ID NO: 4170), A029404673 (SEQ ID NO: 4171), A029404664 (SEQ ID NO: 4172), A029402046 (SEQ ID NO: 4173), A029404670 (SEQ ID NO: 4174), A029404669 (SEQ ID NO: 4175), A029404668 (SEQ ID NO: 4176), A029404667 (SEQ ID NO: 4177), A029404666 (SEQ ID NO: 4178), A029404665 (SEQ ID NO: 4179), A029404663 (SEQ ID NO: 4180), A029404662 (SEQ ID NO: 4181), A029402058 (SEQ ID NO: 4182), A029404661 (SEQ ID NO: 4183), A029404660 (SEQ ID NO: 4184), A029404659 (SEQ ID NO: 4185), A029404658 (SEQ ID NO: 4186), A029404657 (SEQ ID NO: 4187), A029404674 (SEQ ID NO: 4188), A029404675 (SEQ ID NO: 4189), A029404677 (SEQ ID NO: 4190), A029402056 (SEQ ID NO: 4191), A029404676 (SEQ ID NO: 4192), A029404825 (SEQ ID NO: 4193), A029404671 (SEQ ID NO: 4194), A029404824 (SEQ ID NO: 4195), A029404823 (SEQ ID NO: 4196), A029404822 (SEQ ID NO: 4197), A029404821 (SEQ ID NO: 4198), A029404820 (SEQ ID NO: 4199), A029404819 (SEQ ID NO: 4200), A029404818 (SEQ ID NO: 4201), A029404817 (SEQ ID NO: 4202), A029404816 (SEQ ID NO: 4203), A029404815 (SEQ ID NO: 4204), A029404814 (SEQ ID NO: 4205), A029404813 (SEQ ID NO: 4206), A029404812 (SEQ ID NO: 4207), A029404811 (SEQ ID NO: 4208), A029404810 (SEQ ID NO: 4209), A029404809 (SEQ ID NO: 4210), A029404672 (SEQ ID NO: 4211), A029402045 (SEQ ID NO: 4212), A029402043 (SEQ ID NO: 4213), A029402042 (SEQ ID NO: 4214), A029401783 (SEQ ID NO: 4215), A029401779 (SEQ ID NO: 4216), A029401778 (SEQ ID NO: 4217), A029401776 (SEQ ID NO: 4218), A029401775 (SEQ ID NO: 4219), A029401768 (SEQ ID NO: 4220), A029401755 (SEQ ID NO: 4221), A029401830 (SEQ ID NO: 4222), A029401785 (SEQ ID NO: 4223), A029401786 (SEQ ID NO: 4224), A029401746 (SEQ ID NO: 4225), A029401745 (SEQ ID NO: 4226), A029401744 (SEQ ID NO: 4227), A029401743 (SEQ ID NO: 4228), A029401742 (SEQ ID NO: 4229), A029401741 (SEQ ID NO: 4230), A029401784 (SEQ ID NO: 4231), A029401740 (SEQ ID NO: 4232), A029401738 (SEQ ID NO: 4233), A029401748 (SEQ ID NO: 4234), A029400198 (SEQ ID NO: 4235), A029401828 (SEQ ID NO: 4236), A029401824 (SEQ ID NO: 4237), A029401822 (SEQ ID NO: 4238), A029401821 (SEQ ID NO: 4239), A029401820 (SEQ ID NO: 4240), A029401818 (SEQ ID NO: 4241), A029401817 (SEQ ID NO: 4242), A029401816 (SEQ ID NO: 4243), A029401815 (SEQ ID NO: 4244), A029401814 (SEQ ID NO: 4245), A029401807 (SEQ ID NO: 4246), A029401806 (SEQ ID NO: 4247), A029401802 (SEQ ID NO: 4248), A029401796 (SEQ ID NO: 4249), A029401794 (SEQ ID NO: 4250), A029401791 (SEQ ID NO: 4251), A029401747 (SEQ ID NO: 4252), A029401739 (SEQ ID NO: 4253), A029400128 (SEQ ID NO: 4254), A029400129 (SEQ ID NO: 4255), A029400116 (SEQ ID NO: 4256), A029400124 (SEQ ID NO: 4257), A029400123 (SEQ ID NO: 4258), A029400122 (SEQ ID NO: 4259), A029400121 (SEQ ID NO: 4260), A029400120 (SEQ ID NO: 4261), A029400119 (SEQ ID NO: 4262), A029400118 (SEQ ID NO: 4263), A029400126 (SEQ ID NO: 4264), A029401831 (SEQ ID NO: 4265), A029400115 (SEQ ID NO: 4266), A029400114 (SEQ ID NO: 4267), A029400113 (SEQ ID NO: 4268), A029400112 (SEQ ID NO: 4269), A029400052 (SEQ ID NO: 4270), A029400051 (SEQ ID NO: 4271), A029400050 (SEQ ID NO: 4272), A029405023 (SEQ ID NO: 4273), A029405022 (SEQ ID NO: 4274), A029400127 (SEQ ID NO: 4275), A029400131 (SEQ ID NO: 4276), A029400117 (SEQ ID NO: 4277), A029400125 (SEQ ID NO: 4278), A029400190 (SEQ ID NO: 4279), A029400189 (SEQ ID NO: 4280), A029400188 (SEQ ID NO: 4281), A029400187 (SEQ ID NO: 4282), A029400186 (SEQ ID NO: 4283), A029400185 (SEQ ID NO: 4284), A029400184 (SEQ ID NO: 4285), A029400183 (SEQ ID NO: 4286), A029400182 (SEQ ID NO: 4287), A029400181 (SEQ ID NO: 4288), A029400180 (SEQ ID NO: 4289), A029400179 (SEQ ID NO: 4290), A029400134 (SEQ ID NO: 4291), A029400133 (SEQ ID NO: 4292), A029400132 (SEQ ID NO: 4293), A029400130 (SEQ ID NO: 4294), A029404868 (SEQ ID NO: 4295), A029401833 (SEQ ID NO: 4296), A029401852 (SEQ ID NO: 4297), A029401994 (SEQ ID NO: 4298), A029401991 (SEQ ID NO: 4299), A029401982 (SEQ ID NO: 4300), A029401981 (SEQ ID NO: 4301), A029401973 (SEQ ID NO: 4302), A029401972 (SEQ ID NO: 4303), A029401971 (SEQ ID NO: 4304), A029401750 (SEQ ID NO: 4305), A029401970 (SEQ ID NO: 4306), A029401969 (SEQ ID NO: 4307), A029401968 (SEQ ID NO: 4308), A029401966 (SEQ ID NO: 4309), A029401965 (SEQ ID NO: 4310), A029401964 (SEQ ID NO: 4311), A029401955 (SEQ ID NO: 4312), A029401953 (SEQ ID NO: 4313), A029401780 (SEQ ID NO: 4314), A029402041 (SEQ ID NO: 4315), A029402000 (SEQ ID NO: 4316), A029401943 (SEQ ID NO: 4317), A029401942 (SEQ ID NO: 4318), A029402037 (SEQ ID NO: 4319), A029402032 (SEQ ID NO: 4320), A029402027 (SEQ ID NO: 4321), A029402025 (SEQ ID NO: 4322), A029402024 (SEQ ID NO: 4323), A029402023 (SEQ ID NO: 4324), A029402017 (SEQ ID NO: 4325), A029402015 (SEQ ID NO: 4326), A029402013 (SEQ ID NO: 4327), A029402010 (SEQ ID NO: 4328), A029402008 (SEQ ID NO: 4329), A029402007 (SEQ ID NO: 4330), A029402006 (SEQ ID NO: 4331), A029402004 (SEQ ID NO: 4332), A029402003 (SEQ ID NO: 4333), A029402002 (SEQ ID NO: 4334), A029401948 (SEQ ID NO: 4335), A029401987 (SEQ ID NO: 4336), A029401967 (SEQ ID NO: 4337), A029401941 (SEQ ID NO: 4338), A029401847 (SEQ ID NO: 4339), A029401867 (SEQ ID NO: 4340), A029401866 (SEQ ID NO: 4341), A029401884 (SEQ ID NO: 4342), A029401881 (SEQ ID NO: 4343), A029401875 (SEQ ID NO: 4344), A029401874 (SEQ ID NO: 4345), A029401871 (SEQ ID NO: 4346), A029401869 (SEQ ID NO: 4347), A029401863 (SEQ ID NO: 4348), A029401892 (SEQ ID NO: 4349), A029401891 (SEQ ID NO: 4350), A029401862 (SEQ ID NO: 4351), A029401857 (SEQ ID NO: 4352), A029401856 (SEQ ID NO: 4353), A029401855 (SEQ ID NO: 4354), A029401853 (SEQ ID NO: 4355), A029401894 (SEQ ID NO: 4356), A029401848 (SEQ ID NO: 4357), A029401897 (SEQ ID NO: 4358), A029401901 (SEQ ID NO: 4359), A029401940 (SEQ ID NO: 4360), A029401834 (SEQ ID NO: 4361), A029401939 (SEQ ID NO: 4362), A029401889 (SEQ ID NO: 4363), A029401938 (SEQ ID NO: 4364), A029401935 (SEQ ID NO: 4365), A029401934 (SEQ ID NO: 4366), A029401932 (SEQ ID NO: 4367), A029401931 (SEQ ID NO: 4368), A029401930 (SEQ ID NO: 4369), A029401929 (SEQ ID NO: 4370), A029401927 (SEQ ID NO: 4371), A029401922 (SEQ ID NO: 4372), A029401909 (SEQ ID NO: 4373), A029401908 (SEQ ID NO: 4374), A029401906 (SEQ ID NO: 4375), A029401904 (SEQ ID NO: 4376), A029401898 (SEQ ID NO: 4377), A029405038 (SEQ ID NO: 4378) (see Table A-5 and A-10).

In another embodiment, the ISVD specifically binding to human PD-1 may have a sequence identity of more than 90%, such as more than 95% or even more than 99%, with SEQ ID NO: 4049, wherein the CDRs are as defined above. In one embodiment, the ISVD specifically binding to PD-1 comprises or consists of the amino acid sequence of SEQ ID NO: 4049.

In another embodiment, the ISVD specifically binding to human PD-1 may have a sequence identity of more than 90%, such as more than 95% or even more than 99%, with SEQ ID NO: 4050, wherein the CDRs are as defined above. In one embodiment, the ISVD specifically binding to PD-1 comprises or consists of the amino acid sequence of SEQ ID NO: 4050.

In another embodiment, the ISVD specifically binding to human PD-1 may have a sequence identity of more than 90%, such as more than 95% or even more than 99%, with SEQ ID NO: 4051, wherein the CDRs are as defined above. In one embodiment, the ISVD specifically binding to PD-1 comprises or consists of the amino acid sequence of SEQ ID NO: 4051.

In another embodiment, the ISVD specifically binding to human PD-1 may have a sequence identity of more than 90%, such as more than 95% or even more than 99%, with SEQ ID NO: 4052, wherein the CDRs are as defined above. In one embodiment, the ISVD specifically binding to PD-1 comprises or consists of the amino acid sequence of SEQ ID NO: 4052.

In another embodiment, the ISVD specifically binding to human PD-1 may have a sequence identity of more than 90%, such as more than 95% or even more than 99%, with SEQ ID NO: 4053, wherein the CDRs are as defined above. In one embodiment, the ISVD specifically binding to PD-1 comprises or consists of the amino acid sequence of SEQ ID NO: 4053.

In another embodiment, the ISVD specifically binding to human PD-1 may have a sequence identity of more than 90%, such as more than 95% or even more than 99%, with SEQ ID NO: 4054, wherein the CDRs are as defined above. In one embodiment, the ISVD specifically binding to PD-1 comprises or consists of the amino acid sequence of SEQ ID NO: 4054.

In another embodiment, the ISVD specifically binding to human PD-1 may have a sequence identity of more than 90%, such as more than 95% or even more than 99%, with SEQ ID NO: 4055, wherein the CDRs are as defined above. In one embodiment, the ISVD specifically binding to PD-1 comprises or consists of the amino acid sequence of SEQ ID NO: 4055.

In another embodiment, the ISVD specifically binding to human PD-1 may have a sequence identity of more than 90%, such as more than 95% or even more than 99%, with SEQ ID NO: 4056, wherein the CDRs are as defined above. In one embodiment, the ISVD specifically binding to PD-1 comprises or consists of the amino acid sequence of SEQ ID NO: 4056.

In another embodiment, the ISVD specifically binding to human PD-1 may have a sequence identity of more than 90%, such as more than 95% or even more than 99%, with SEQ ID NO: 4057, wherein the CDRs are as defined above. In one embodiment, the ISVD specifically binding to PD-1 comprises or consists of the amino acid sequence of SEQ ID NO: 4057.

In another embodiment, the ISVD specifically binding to human PD-1 may have a sequence identity of more than 90%, such as more than 95% or even more than 99%, with SEQ ID NO: 4058, wherein the CDRs are as defined above. In one embodiment, the ISVD specifically binding to PD-1 comprises or consists of the amino acid sequence of SEQ ID NO: 4058.

In another embodiment, the ISVD specifically binding to human PD-1 may have a sequence identity of more than 90%, such as more than 95% or even more than 99%, with SEQ ID NO: 4059, wherein the CDRs are as defined above. In one embodiment, the ISVD specifically binding to PD-1 comprises or consists of the amino acid sequence of SEQ ID NO: 4059.

In another embodiment, the ISVD specifically binding to human PD-1 may have a sequence identity of more than 90%, such as more than 95% or even more than 99%, with SEQ ID NO: 4060, wherein the CDRs are as defined above. In one embodiment, the ISVD specifically binding to PD-1 comprises or consists of the amino acid sequence of SEQ ID NO: 4060.

In another embodiment, the ISVD specifically binding to human PD-1 may have a sequence identity of more than 90%, such as more than 95% or even more than 99%, with SEQ ID NO: 4061, wherein the CDRs are as defined above. In one embodiment, the ISVD specifically binding to PD-1 comprises or consists of the amino acid sequence of SEQ ID NO: 4061.

In another embodiment, the ISVD specifically binding to human PD-1 may have a sequence identity of more than 90%, such as more than 95% or even more than 99%, with SEQ ID NO: 4062, wherein the CDRs are as defined above. In one embodiment, the ISVD specifically binding to PD-1 comprises or consists of the amino acid sequence of SEQ ID NO: 4062.

In another embodiment, the ISVD specifically binding to human PD-1 may have a sequence identity of more than 90%, such as more than 95% or even more than 99%, with SEQ ID NO: 4063, wherein the CDRs are as defined above. In one embodiment, the ISVD specifically binding to PD-1 comprises or consists of the amino acid sequence of SEQ ID NO: 4063.

In another embodiment, the ISVD specifically binding to human PD-1 may have a sequence identity of more than 90%, such as more than 95% or even more than 99%, with SEQ ID NO: 4064, wherein the CDRs are as defined above. In one embodiment, the ISVD specifically binding to PD-1 comprises or consists of the amino acid sequence of SEQ ID NO: 4064.

In another embodiment, the ISVD specifically binding to human PD-1 may have a sequence identity of more than 90%, such as more than 95% or even more than 99%, with SEQ ID NO: 4065, wherein the CDRs are as defined above. In one embodiment, the ISVD specifically binding to PD-1 comprises or consists of the amino acid sequence of SEQ ID NO: 4065.

In another embodiment, the ISVD specifically binding to human PD-1 may have a sequence identity of more than 90%, such as more than 95% or even more than 99%, with SEQ ID NO: 4066, wherein the CDRs are as defined above. In one embodiment, the ISVD specifically binding to PD-1 comprises or consists of the amino acid sequence of SEQ ID NO: 4066.

In another embodiment, the ISVD specifically binding to human PD-1 may have a sequence identity of more than 90%, such as more than 95% or even more than 99%, with SEQ ID NO: 4067, wherein the CDRs are as defined above. In one embodiment, the ISVD specifically binding to PD-1 comprises or consists of the amino acid sequence of SEQ ID NO: 4067.

In another embodiment, the ISVD specifically binding to human PD-1 may have a sequence identity of more than 90%, such as more than 95% or even more than 99%, with SEQ ID NO: 4068, wherein the CDRs are as defined above. In one embodiment, the ISVD specifically binding to PD-1 comprises or consists of the amino acid sequence of SEQ ID NO: 4068.

In another embodiment, the ISVD specifically binding to human PD-1 may have a sequence identity of more than 90%, such as more than 95% or even more than 99%, with SEQ ID NO: 4069, wherein the CDRs are as defined above. In one embodiment, the ISVD specifically binding to PD-1 comprises or consists of the amino acid sequence of SEQ ID NO: 4069.

In another embodiment, the ISVD specifically binding to human PD-1 may have a sequence identity of more than 90%, such as more than 95% or even more than 99%, with SEQ ID NO: 4070, wherein the CDRs are as defined above. In one embodiment, the ISVD specifically binding to PD-1 comprises or consists of the amino acid sequence of SEQ ID NO: 4070.

In another embodiment, the ISVD specifically binding to human PD-1 may have a sequence identity of more than 90%, such as more than 95% or even more than 99%, with SEQ ID NO: 4071, wherein the CDRs are as defined above. In one embodiment, the ISVD specifically binding to PD-1 comprises or consists of the amino acid sequence of SEQ ID NO: 4071.

In another embodiment, the ISVD specifically binding to human PD-1 may have a sequence identity of more than 90%, such as more than 95% or even more than 99%, with SEQ ID NO: 4072, wherein the CDRs are as defined above. In one embodiment, the ISVD specifically binding to PD-1 comprises or consists of the amino acid sequence of SEQ ID NO: 4072.

In another embodiment, the ISVD specifically binding to human PD-1 may have a sequence identity of more than 90%, such as more than 95% or even more than 99%, with SEQ ID NO: 4073, wherein the CDRs are as defined above. In one embodiment, the ISVD specifically binding to PD-1 comprises or consists of the amino acid sequence of SEQ ID NO: 4073.

In another embodiment, the ISVD specifically binding to human PD-1 may have a sequence identity of more than 90%, such as more than 95% or even more than 99%, with SEQ ID NO: 4074, wherein the CDRs are as defined above. In one embodiment, the ISVD specifically binding to PD-1 comprises or consists of the amino acid sequence of SEQ ID NO: 4074.

In another embodiment, the ISVD specifically binding to human PD-1 may have a sequence identity of more than 90%, such as more than 95% or even more than 99%, with SEQ ID NO: 4075, wherein the CDRs are as defined above. In one embodiment, the ISVD specifically binding to PD-1 comprises or consists of the amino acid sequence of SEQ ID NO: 4075.

In another embodiment, the ISVD specifically binding to human PD-1 may have a sequence identity of more than 90%, such as more than 95% or even more than 99%, with SEQ ID NO: 4076, wherein the CDRs are as defined above. In one embodiment, the ISVD specifically binding to PD-1 comprises or consists of the amino acid sequence of SEQ ID NO: 4076.

In another embodiment, the ISVD specifically binding to human PD-1 may have a sequence identity of more than 90%, such as more than 95% or even more than 99%, with SEQ ID NO: 4077, wherein the CDRs are as defined above. In one embodiment, the ISVD specifically binding to PD-1 comprises or consists of the amino acid sequence of SEQ ID NO: 4077.

In another embodiment, the ISVD specifically binding to human PD-1 may have a sequence identity of more than 90%, such as more than 95% or even more than 99%, with SEQ ID NO: 4078, wherein the CDRs are as defined above. In one embodiment, the ISVD specifically binding to PD-1 comprises or consists of the amino acid sequence of SEQ ID NO: 4078.

In another embodiment, the ISVD specifically binding to human PD-1 may have a sequence identity of more than 90%, such as more than 95% or even more than 99%, with SEQ ID NO: 4079, wherein the CDRs are as defined above. In one embodiment, the ISVD specifically binding to PD-1 comprises or consists of the amino acid sequence of SEQ ID NO: 4079.

In another embodiment, the ISVD specifically binding to human PD-1 may have a sequence identity of more than 90%, such as more than 95% or even more than 99%, with SEQ ID NO: 4080, wherein the CDRs are as defined above. In one embodiment, the ISVD specifically binding to PD-1 comprises or consists of the amino acid sequence of SEQ ID NO: 4080.

In another embodiment, the ISVD specifically binding to human PD-1 may have a sequence identity of more than 90%, such as more than 95% or even more than 99%, with SEQ ID NO: 4081, wherein the CDRs are as defined above. In one embodiment, the ISVD specifically binding to PD-1 comprises or consists of the amino acid sequence of SEQ ID NO: 4081.

In another embodiment, the ISVD specifically binding to human PD-1 may have a sequence identity of more than 90%, such as more than 95% or even more than 99%, with SEQ ID NO: 4082, wherein the CDRs are as defined above. In one embodiment, the ISVD specifically binding to PD-1 comprises or consists of the amino acid sequence of SEQ ID NO: 4082.

In another embodiment, the ISVD specifically binding to human PD-1 may have a sequence identity of more than 90%, such as more than 95% or even more than 99%, with SEQ ID NO: 4083, wherein the CDRs are as defined above. In one embodiment, the ISVD specifically binding to PD-1 comprises or consists of the amino acid sequence of SEQ ID NO: 4083.

In another embodiment, the ISVD specifically binding to human PD-1 may have a sequence identity of more than 90%, such as more than 95% or even more than 99%, with SEQ ID NO: 4084, wherein the CDRs are as defined above. In one embodiment, the ISVD specifically binding to PD-1 comprises or consists of the amino acid sequence of SEQ ID NO: 4084.

In another embodiment, the ISVD specifically binding to human PD-1 may have a sequence identity of more than 90%, such as more than 95% or even more than 99%, with SEQ ID NO: 4085, wherein the CDRs are as defined above. In one embodiment, the ISVD specifically binding to PD-1 comprises or consists of the amino acid sequence of SEQ ID NO: 4085.

In another embodiment, the ISVD specifically binding to human PD-1 may have a sequence identity of more than 90%, such as more than 95% or even more than 99%, with SEQ ID NO: 4086, wherein the CDRs are as defined above. In one embodiment, the ISVD specifically binding to PD-1 comprises or consists of the amino acid sequence of SEQ ID NO: 4086.

In another embodiment, the ISVD specifically binding to human PD-1 may have a sequence identity of more than 90%, such as more than 95% or even more than 99%, with SEQ ID NO: 4087, wherein the CDRs are as defined above. In one embodiment, the ISVD specifically binding to PD-1 comprises or consists of the amino acid sequence of SEQ ID NO: 4087.

In another embodiment, the ISVD specifically binding to human PD-1 may have a sequence identity of more than 90%, such as more than 95% or even more than 99%, with SEQ ID NO: 4088, wherein the CDRs are as defined above. In one embodiment, the ISVD specifically binding to PD-1 comprises or consists of the amino acid sequence of SEQ ID NO: 4088.

In another embodiment, the ISVD specifically binding to human PD-1 may have a sequence identity of more than 90%, such as more than 95% or even more than 99%, with SEQ ID NO: 4089, wherein the CDRs are as defined above. In one embodiment, the ISVD specifically binding to PD-1 comprises or consists of the amino acid sequence of SEQ ID NO: 4089.

In another embodiment, the ISVD specifically binding to human PD-1 may have a sequence identity of more than 90%, such as more than 95% or even more than 99%, with SEQ ID NO: 4090, wherein the CDRs are as defined above. In one embodiment, the ISVD specifically binding to PD-1 comprises or consists of the amino acid sequence of SEQ ID NO: 4090.

In another embodiment, the ISVD specifically binding to human PD-1 may have a sequence identity of more than 90%, such as more than 95% or even more than 99%, with SEQ ID NO: 4091, wherein the CDRs are as defined above. In one embodiment, the ISVD specifically binding to PD-1 comprises or consists of the amino acid sequence of SEQ ID NO: 4091.

In another embodiment, the ISVD specifically binding to human PD-1 may have a sequence identity of more than 90%, such as more than 95% or even more than 99%, with SEQ ID NO: 4092, wherein the CDRs are as defined above. In one embodiment, the ISVD specifically binding to PD-1 comprises or consists of the amino acid sequence of SEQ ID NO: 4092.

In another embodiment, the ISVD specifically binding to human PD-1 may have a sequence identity of more than 90%, such as more than 95% or even more than 99%, with SEQ ID NO: 4093, wherein the CDRs are as defined above. In one embodiment, the ISVD specifically binding to PD-1 comprises or consists of the amino acid sequence of SEQ ID NO: 4093.

In another embodiment, the ISVD specifically binding to human PD-1 may have a sequence identity of more than 90%, such as more than 95% or even more than 99%, with SEQ ID NO: 4094, wherein the CDRs are as defined above. In one embodiment, the ISVD specifically binding to PD-1 comprises or consists of the amino acid sequence of SEQ ID NO: 4094.

In another embodiment, the ISVD specifically binding to human PD-1 may have a sequence identity of more than 90%, such as more than 95% or even more than 99%, with SEQ ID NO: 4095, wherein the CDRs are as defined above. In one embodiment, the ISVD specifically binding to PD-1 comprises or consists of the amino acid sequence of SEQ ID NO: 4095.

In another embodiment, the ISVD specifically binding to human PD-1 may have a sequence identity of more than 90%, such as more than 95% or even more than 99%, with SEQ ID NO: 4096, wherein the CDRs are as defined above. In one embodiment, the ISVD specifically binding to PD-1 comprises or consists of the amino acid sequence of SEQ ID NO: 4096.

In another embodiment, the ISVD specifically binding to human PD-1 may have a sequence identity of more than 90%, such as more than 95% or even more than 99%, with SEQ ID NO: 4097, wherein the CDRs are as defined above. In one embodiment, the ISVD specifically binding to PD-1 comprises or consists of the amino acid sequence of SEQ ID NO: 4097.

In another embodiment, the ISVD specifically binding to human PD-1 may have a sequence identity of more than 90%, such as more than 95% or even more than 99%, with SEQ ID NO: 4098, wherein the CDRs are as defined above. In one embodiment, the ISVD specifically binding to PD-1 comprises or consists of the amino acid sequence of SEQ ID NO: 4098.

In another embodiment, the ISVD specifically binding to human PD-1 may have a sequence identity of more than 90%, such as more than 95% or even more than 99%, with SEQ ID NO: 4099, wherein the CDRs are as defined above. In one embodiment, the ISVD specifically binding to PD-1 comprises or consists of the amino acid sequence of SEQ ID NO: 4099.

In another embodiment, the ISVD specifically binding to human PD-1 may have a sequence identity of more than 90%, such as more than 95% or even more than 99%, with SEQ ID NO: 4100, wherein the CDRs are as defined above. In one embodiment, the ISVD specifically binding to PD-1 comprises or consists of the amino acid sequence of SEQ ID NO: 4100.

In another embodiment, the ISVD specifically binding to human PD-1 may have a sequence identity of more than 90%, such as more than 95% or even more than 99%, with SEQ ID NO: 4101, wherein the CDRs are as defined above. In one embodiment, the ISVD specifically binding to PD-1 comprises or consists of the amino acid sequence of SEQ ID NO: 4101.

In another embodiment, the ISVD specifically binding to human PD-1 may have a sequence identity of more than 90%, such as more than 95% or even more than 99%, with SEQ ID NO: 4102, wherein the CDRs are as defined above. In one embodiment, the ISVD specifically binding to PD-1 comprises or consists of the amino acid sequence of SEQ ID NO: 4102.

In another embodiment, the ISVD specifically binding to human PD-1 may have a sequence identity of more than 90%, such as more than 95% or even more than 99%, with SEQ ID NO: 4103, wherein the CDRs are as defined above. In one embodiment, the ISVD specifically binding to PD-1 comprises or consists of the amino acid sequence of SEQ ID NO: 4103.

In another embodiment, the ISVD specifically binding to human PD-1 may have a sequence identity of more than 90%, such as more than 95% or even more than 99%, with SEQ ID NO: 4104, wherein the CDRs are as defined above. In one embodiment, the ISVD specifically binding to PD-1 comprises or consists of the amino acid sequence of SEQ ID NO: 4104.

In another embodiment, the ISVD specifically binding to human PD-1 may have a sequence identity of more than 90%, such as more than 95% or even more than 99%, with SEQ ID NO: 4105, wherein the CDRs are as defined above. In one embodiment, the ISVD specifically binding to PD-1 comprises or consists of the amino acid sequence of SEQ ID NO: 4105.

In another embodiment, the ISVD specifically binding to human PD-1 may have a sequence identity of more than 90%, such as more than 95% or even more than 99%, with SEQ ID NO: 4106, wherein the CDRs are as defined above. In one embodiment, the ISVD specifically binding to PD-1 comprises or consists of the amino acid sequence of SEQ ID NO: 4106.

In another embodiment, the ISVD specifically binding to human PD-1 may have a sequence identity of more than 90%, such as more than 95% or even more than 99%, with SEQ ID NO: 4107, wherein the CDRs are as defined above. In one embodiment, the ISVD specifically binding to PD-1 comprises or consists of the amino acid sequence of SEQ ID NO: 4107.

In another embodiment, the ISVD specifically binding to human PD-1 may have a sequence identity of more than 90%, such as more than 95% or even more than 99%, with SEQ ID NO: 4108, wherein the CDRs are as defined above. In one embodiment, the ISVD specifically binding to PD-1 comprises or consists of the amino acid sequence of SEQ ID NO: 4108.

In another embodiment, the ISVD specifically binding to human PD-1 may have a sequence identity of more than 90%, such as more than 95% or even more than 99%, with SEQ ID NO: 4109, wherein the CDRs are as defined above. In one embodiment, the ISVD specifically binding to PD-1 comprises or consists of the amino acid sequence of SEQ ID NO: 4109.

In another embodiment, the ISVD specifically binding to human PD-1 may have a sequence identity of more than 90%, such as more than 95% or even more than 99%, with SEQ ID NO: 4110, wherein the CDRs are as defined above. In one embodiment, the ISVD specifically binding to PD-1 comprises or consists of the amino acid sequence of SEQ ID NO: 4110.

In another embodiment, the ISVD specifically binding to human PD-1 may have a sequence identity of more than 90%, such as more than 95% or even more than 99%, with SEQ ID NO: 4111, wherein the CDRs are as defined above. In one embodiment, the ISVD specifically binding to PD-1 comprises or consists of the amino acid sequence of SEQ ID NO: 4111.

In another embodiment, the ISVD specifically binding to human PD-1 may have a sequence identity of more than 90%, such as more than 95% or even more than 99%, with SEQ ID NO: 4112, wherein the CDRs are as defined above. In one embodiment, the ISVD specifically binding to PD-1 comprises or consists of the amino acid sequence of SEQ ID NO: 4112.

In another embodiment, the ISVD specifically binding to human PD-1 may have a sequence identity of more than 90%, such as more than 95% or even more than 99%, with SEQ ID NO: 4113, wherein the CDRs are as defined above. In one embodiment, the ISVD specifically binding to PD-1 comprises or consists of the amino acid sequence of SEQ ID NO: 4113.

In another embodiment, the ISVD specifically binding to human PD-1 may have a sequence identity of more than 90%, such as more than 95% or even more than 99%, with SEQ ID NO: 4114, wherein the CDRs are as defined above. In one embodiment, the ISVD specifically binding to PD-1 comprises or consists of the amino acid sequence of SEQ ID NO: 4114.

In another embodiment, the ISVD specifically binding to human PD-1 may have a sequence identity of more than 90%, such as more than 95% or even more than 99%, with SEQ ID NO: 4115, wherein the CDRs are as defined above. In one embodiment, the ISVD specifically binding to PD-1 comprises or consists of the amino acid sequence of SEQ ID NO: 4115.

In another embodiment, the ISVD specifically binding to human PD-1 may have a sequence identity of more than 90%, such as more than 95% or even more than 99%, with SEQ ID NO: 4116, wherein the CDRs are as defined above. In one embodiment, the ISVD specifically binding to PD-1 comprises or consists of the amino acid sequence of SEQ ID NO: 4116.

In another embodiment, the ISVD specifically binding to human PD-1 may have a sequence identity of more than 90%, such as more than 95% or even more than 99%, with SEQ ID NO: 4117, wherein the CDRs are as defined above. In one embodiment, the ISVD specifically binding to PD-1 comprises or consists of the amino acid sequence of SEQ ID NO: 4117.

In another embodiment, the ISVD specifically binding to human PD-1 may have a sequence identity of more than 90%, such as more than 95% or even more than 99%, with SEQ ID NO: 4118, wherein the CDRs are as defined above. In one embodiment, the ISVD specifically binding to PD-1 comprises or consists of the amino acid sequence of SEQ ID NO: 4118.

In another embodiment, the ISVD specifically binding to human PD-1 may have a sequence identity of more than 90%, such as more than 95% or even more than 99%, with SEQ ID NO: 4119, wherein the CDRs are as defined above. In one embodiment, the ISVD specifically binding to PD-1 comprises or consists of the amino acid sequence of SEQ ID NO: 4119.

In another embodiment, the ISVD specifically binding to human PD-1 may have a sequence identity of more than 90%, such as more than 95% or even more than 99%, with SEQ ID NO: 4120, wherein the CDRs are as defined above. In one embodiment, the ISVD specifically binding to PD-1 comprises or consists of the amino acid sequence of SEQ ID NO: 4120.

In another embodiment, the ISVD specifically binding to human PD-1 may have a sequence identity of more than 90%, such as more than 95% or even more than 99%, with SEQ ID NO: 4121, wherein the CDRs are as defined above. In one embodiment, the ISVD specifically binding to PD-1 comprises or consists of the amino acid sequence of SEQ ID NO: 4121.

In another embodiment, the ISVD specifically binding to human PD-1 may have a sequence identity of more than 90%, such as more than 95% or even more than 99%, with SEQ ID NO: 4122, wherein the CDRs are as defined above. In one embodiment, the ISVD specifically binding to PD-1 comprises or consists of the amino acid sequence of SEQ ID NO: 4122.

In another embodiment, the ISVD specifically binding to human PD-1 may have a sequence identity of more than 90%, such as more than 95% or even more than 99%, with SEQ ID NO: 4123, wherein the CDRs are as defined above. In one embodiment, the ISVD specifically binding to PD-1 comprises or consists of the amino acid sequence of SEQ ID NO: 4123.

In another embodiment, the ISVD specifically binding to human PD-1 may have a sequence identity of more than 90%, such as more than 95% or even more than 99%, with SEQ ID NO: 4124, wherein the CDRs are as defined above. In one embodiment, the ISVD specifically binding to PD-1 comprises or consists of the amino acid sequence of SEQ ID NO: 4124.

In another embodiment, the ISVD specifically binding to human PD-1 may have a sequence identity of more than 90%, such as more than 95% or even more than 99%, with SEQ ID NO: 4125, wherein the CDRs are as defined above. In one embodiment, the ISVD specifically binding to PD-1 comprises or consists of the amino acid sequence of SEQ ID NO: 4125.

In another embodiment, the ISVD specifically binding to human PD-1 may have a sequence identity of more than 90%, such as more than 95% or even more than 99%, with SEQ ID NO: 4126, wherein the CDRs are as defined above. In one embodiment, the ISVD specifically binding to PD-1 comprises or consists of the amino acid sequence of SEQ ID NO: 4126.

In another embodiment, the ISVD specifically binding to human PD-1 may have a sequence identity of more than 90%, such as more than 95% or even more than 99%, with SEQ ID NO: 4127, wherein the CDRs are as defined above. In one embodiment, the ISVD specifically binding to PD-1 comprises or consists of the amino acid sequence of SEQ ID NO: 4127.

In another embodiment, the ISVD specifically binding to human PD-1 may have a sequence identity of more than 90%, such as more than 95% or even more than 99%, with SEQ ID NO: 4128, wherein the CDRs are as defined above. In one embodiment, the ISVD specifically binding to PD-1 comprises or consists of the amino acid sequence of SEQ ID NO: 4128.

In another embodiment, the ISVD specifically binding to human PD-1 may have a sequence identity of more than 90%, such as more than 95% or even more than 99%, with SEQ ID NO: 4129, wherein the CDRs are as defined above. In one embodiment, the ISVD specifically binding to PD-1 comprises or consists of the amino acid sequence of SEQ ID NO: 4129.

In another embodiment, the ISVD specifically binding to human PD-1 may have a sequence identity of more than 90%, such as more than 95% or even more than 99%, with SEQ ID NO: 4130, wherein the CDRs are as defined above. In one embodiment, the ISVD specifically binding to PD-1 comprises or consists of the amino acid sequence of SEQ ID NO: 4130.

In another embodiment, the ISVD specifically binding to human PD-1 may have a sequence identity of more than 90%, such as more than 95% or even more than 99%, with SEQ ID NO: 4131, wherein the CDRs are as defined above. In one embodiment, the ISVD specifically binding to PD-1 comprises or consists of the amino acid sequence of SEQ ID NO: 4131.

In another embodiment, the ISVD specifically binding to human PD-1 may have a sequence identity of more than 90%, such as more than 95% or even more than 99%, with SEQ ID NO: 4132, wherein the CDRs are as defined above. In one embodiment, the ISVD specifically binding to PD-1 comprises or consists of the amino acid sequence of SEQ ID NO: 4132.

In another embodiment, the ISVD specifically binding to human PD-1 may have a sequence identity of more than 90%, such as more than 95% or even more than 99%, with SEQ ID NO: 4133, wherein the CDRs are as defined above. In one embodiment, the ISVD specifically binding to PD-1 comprises or consists of the amino acid sequence of SEQ ID NO: 4133.

In another embodiment, the ISVD specifically binding to human PD-1 may have a sequence identity of more than 90%, such as more than 95% or even more than 99%, with SEQ ID NO: 4134, wherein the CDRs are as defined above. In one embodiment, the ISVD specifically binding to PD-1 comprises or consists of the amino acid sequence of SEQ ID NO: 4134.

In another embodiment, the ISVD specifically binding to human PD-1 may have a sequence identity of more than 90%, such as more than 95% or even more than 99%, with SEQ ID NO: 4135, wherein the CDRs are as defined above. In one embodiment, the ISVD specifically binding to PD-1 comprises or consists of the amino acid sequence of SEQ ID NO: 4135.

In another embodiment, the ISVD specifically binding to human PD-1 may have a sequence identity of more than 90%, such as more than 95% or even more than 99%, with SEQ ID NO: 4136, wherein the CDRs are as defined above. In one embodiment, the ISVD specifically binding to PD-1 comprises or consists of the amino acid sequence of SEQ ID NO: 4136.

In another embodiment, the ISVD specifically binding to human PD-1 may have a sequence identity of more than 90%, such as more than 95% or even more than 99%, with SEQ ID NO: 4137, wherein the CDRs are as defined above. In one embodiment, the ISVD specifically binding to PD-1 comprises or consists of the amino acid sequence of SEQ ID NO: 4137.

In another embodiment, the ISVD specifically binding to human PD-1 may have a sequence identity of more than 90%, such as more than 95% or even more than 99%, with SEQ ID NO: 4138, wherein the CDRs are as defined above. In one embodiment, the ISVD specifically binding to PD-1 comprises or consists of the amino acid sequence of SEQ ID NO: 4138.

In another embodiment, the ISVD specifically binding to human PD-1 may have a sequence identity of more than 90%, such as more than 95% or even more than 99%, with SEQ ID NO: 4139, wherein the CDRs are as defined above. In one embodiment, the ISVD specifically binding to PD-1 comprises or consists of the amino acid sequence of SEQ ID NO: 4139.

In another embodiment, the ISVD specifically binding to human PD-1 may have a sequence identity of more than 90%, such as more than 95% or even more than 99%, with SEQ ID NO: 4140, wherein the CDRs are as defined above. In one embodiment, the ISVD specifically binding to PD-1 comprises or consists of the amino acid sequence of SEQ ID NO: 4140.

In another embodiment, the ISVD specifically binding to human PD-1 may have a sequence identity of more than 90%, such as more than 95% or even more than 99%, with SEQ ID NO: 4141, wherein the CDRs are as defined above. In one embodiment, the ISVD specifically binding to PD-1 comprises or consists of the amino acid sequence of SEQ ID NO: 4141.

In another embodiment, the ISVD specifically binding to human PD-1 may have a sequence identity of more than 90%, such as more than 95% or even more than 99%, with SEQ ID NO: 4142, wherein the CDRs are as defined above. In one embodiment, the ISVD specifically binding to PD-1 comprises or consists of the amino acid sequence of SEQ ID NO: 4142.

In another embodiment, the ISVD specifically binding to human PD-1 may have a sequence identity of more than 90%, such as more than 95% or even more than 99%, with SEQ ID NO: 4143, wherein the CDRs are as defined above. In one embodiment, the ISVD specifically binding to PD-1 comprises or consists of the amino acid sequence of SEQ ID NO: 4143.

In another embodiment, the ISVD specifically binding to human PD-1 may have a sequence identity of more than 90%, such as more than 95% or even more than 99%, with SEQ ID NO: 4144, wherein the CDRs are as defined above. In one embodiment, the ISVD specifically binding to PD-1 comprises or consists of the amino acid sequence of SEQ ID NO: 4144.

In another embodiment, the ISVD specifically binding to human PD-1 may have a sequence identity of more than 90%, such as more than 95% or even more than 99%, with SEQ ID NO: 4145, wherein the CDRs are as defined above. In one embodiment, the ISVD specifically binding to PD-1 comprises or consists of the amino acid sequence of SEQ ID NO: 4145.

In another embodiment, the ISVD specifically binding to human PD-1 may have a sequence identity of more than 90%, such as more than 95% or even more than 99%, with SEQ ID NO: 4146, wherein the CDRs are as defined above. In one embodiment, the ISVD specifically binding to PD-1 comprises or consists of the amino acid sequence of SEQ ID NO: 4146.

In another embodiment, the ISVD specifically binding to human PD-1 may have a sequence identity of more than 90%, such as more than 95% or even more than 99%, with SEQ ID NO: 4147, wherein the CDRs are as defined above. In one embodiment, the ISVD specifically binding to PD-1 comprises or consists of the amino acid sequence of SEQ ID NO: 4147.

In another embodiment, the ISVD specifically binding to human PD-1 may have a sequence identity of more than 90%, such as more than 95% or even more than 99%, with SEQ ID NO: 4148, wherein the CDRs are as defined above. In one embodiment, the ISVD specifically binding to PD-1 comprises or consists of the amino acid sequence of SEQ ID NO: 4148.

In another embodiment, the ISVD specifically binding to human PD-1 may have a sequence identity of more than 90%, such as more than 95% or even more than 99%, with SEQ ID NO: 4149, wherein the CDRs are as defined above. In one embodiment, the ISVD specifically binding to PD-1 comprises or consists of the amino acid sequence of SEQ ID NO: 4149.

In another embodiment, the ISVD specifically binding to human PD-1 may have a sequence identity of more than 90%, such as more than 95% or even more than 99%, with SEQ ID NO: 4150, wherein the CDRs are as defined above. In one embodiment, the ISVD specifically binding to PD-1 comprises or consists of the amino acid sequence of SEQ ID NO: 4150.

In another embodiment, the ISVD specifically binding to human PD-1 may have a sequence identity of more than 90%, such as more than 95% or even more than 99%, with SEQ ID NO: 4151, wherein the CDRs are as defined above. In one embodiment, the ISVD specifically binding to PD-1 comprises or consists of the amino acid sequence of SEQ ID NO: 4151.

In another embodiment, the ISVD specifically binding to human PD-1 may have a sequence identity of more than 90%, such as more than 95% or even more than 99%, with SEQ ID NO: 4152, wherein the CDRs are as defined above. In one embodiment, the ISVD specifically binding to PD-1 comprises or consists of the amino acid sequence of SEQ ID NO: 4152.

In another embodiment, the ISVD specifically binding to human PD-1 may have a sequence identity of more than 90%, such as more than 95% or even more than 99%, with SEQ ID NO: 4153, wherein the CDRs are as defined above. In one embodiment, the ISVD specifically binding to PD-1 comprises or consists of the amino acid sequence of SEQ ID NO: 4153.

In another embodiment, the ISVD specifically binding to human PD-1 may have a sequence identity of more than 90%, such as more than 95% or even more than 99%, with SEQ ID NO: 4154, wherein the CDRs are as defined above. In one embodiment, the ISVD specifically binding to PD-1 comprises or consists of the amino acid sequence of SEQ ID NO: 4154.

In another embodiment, the ISVD specifically binding to human PD-1 may have a sequence identity of more than 90%, such as more than 95% or even more than 99%, with SEQ ID NO: 4155, wherein the CDRs are as defined above. In one embodiment, the ISVD specifically binding to PD-1 comprises or consists of the amino acid sequence of SEQ ID NO: 4155.

In another embodiment, the ISVD specifically binding to human PD-1 may have a sequence identity of more than 90%, such as more than 95% or even more than 99%, with SEQ ID NO: 4156, wherein the CDRs are as defined above. In one embodiment, the ISVD specifically binding to PD-1 comprises or consists of the amino acid sequence of SEQ ID NO: 4156.

In another embodiment, the ISVD specifically binding to human PD-1 may have a sequence identity of more than 90%, such as more than 95% or even more than 99%, with SEQ ID NO: 4157, wherein the CDRs are as defined above. In one embodiment, the ISVD specifically binding to PD-1 comprises or consists of the amino acid sequence of SEQ ID NO: 4157.

In another embodiment, the ISVD specifically binding to human PD-1 may have a sequence identity of more than 90%, such as more than 95% or even more than 99%, with SEQ ID NO: 4158, wherein the CDRs are as defined above. In one embodiment, the ISVD specifically binding to PD-1 comprises or consists of the amino acid sequence of SEQ ID NO: 4158.

In another embodiment, the ISVD specifically binding to human PD-1 may have a sequence identity of more than 90%, such as more than 95% or even more than 99%, with SEQ ID NO: 4159, wherein the CDRs are as defined above. In one embodiment, the ISVD specifically binding to PD-1 comprises or consists of the amino acid sequence of SEQ ID NO: 4159.

In another embodiment, the ISVD specifically binding to human PD-1 may have a sequence identity of more than 90%, such as more than 95% or even more than 99%, with SEQ ID NO: 4160, wherein the CDRs are as defined above. In one embodiment, the ISVD specifically binding to PD-1 comprises or consists of the amino acid sequence of SEQ ID NO: 4160.

In another embodiment, the ISVD specifically binding to human PD-1 may have a sequence identity of more than 90%, such as more than 95% or even more than 99%, with SEQ ID NO: 4161, wherein the CDRs are as defined above. In one embodiment, the ISVD specifically binding to PD-1 comprises or consists of the amino acid sequence of SEQ ID NO: 4161.

In another embodiment, the ISVD specifically binding to human PD-1 may have a sequence identity of more than 90%, such as more than 95% or even more than 99%, with SEQ ID NO: 4162, wherein the CDRs are as defined above. In one embodiment, the ISVD specifically binding to PD-1 comprises or consists of the amino acid sequence of SEQ ID NO: 4162.

In another embodiment, the ISVD specifically binding to human PD-1 may have a sequence identity of more than 90%, such as more than 95% or even more than 99%, with SEQ ID NO: 4163, wherein the CDRs are as defined above. In one embodiment, the ISVD specifically binding to PD-1 comprises or consists of the amino acid sequence of SEQ ID NO: 4163.

In another embodiment, the ISVD specifically binding to human PD-1 may have a sequence identity of more than 90%, such as more than 95% or even more than 99%, with SEQ ID NO: 4164, wherein the CDRs are as defined above. In one embodiment, the ISVD specifically binding to PD-1 comprises or consists of the amino acid sequence of SEQ ID NO: 4164.

In another embodiment, the ISVD specifically binding to human PD-1 may have a sequence identity of more than 90%, such as more than 95% or even more than 99%, with SEQ ID NO: 4165, wherein the CDRs are as defined above. In one embodiment, the ISVD specifically binding to PD-1 comprises or consists of the amino acid sequence of SEQ ID NO: 4165.

In another embodiment, the ISVD specifically binding to human PD-1 may have a sequence identity of more than 90%, such as more than 95% or even more than 99%, with SEQ ID NO: 4166, wherein the CDRs are as defined above. In one embodiment, the ISVD specifically binding to PD-1 comprises or consists of the amino acid sequence of SEQ ID NO: 4166.

In another embodiment, the ISVD specifically binding to human PD-1 may have a sequence identity of more than 90%, such as more than 95% or even more than 99%, with SEQ ID NO: 4167, wherein the CDRs are as defined above. In one embodiment, the ISVD specifically binding to PD-1 comprises or consists of the amino acid sequence of SEQ ID NO: 4167.

In another embodiment, the ISVD specifically binding to human PD-1 may have a sequence identity of more than 90%, such as more than 95% or even more than 99%, with SEQ ID NO: 4168, wherein the CDRs are as defined above. In one embodiment, the ISVD specifically binding to PD-1 comprises or consists of the amino acid sequence of SEQ ID NO: 4168.

In another embodiment, the ISVD specifically binding to human PD-1 may have a sequence identity of more than 90%, such as more than 95% or even more than 99%, with SEQ ID NO: 4169, wherein the CDRs are as defined above. In one embodiment, the ISVD specifically binding to PD-1 comprises or consists of the amino acid sequence of SEQ ID NO: 4169.

In another embodiment, the ISVD specifically binding to human PD-1 may have a sequence identity of more than 90%, such as more than 95% or even more than 99%, with SEQ ID NO: 4170, wherein the CDRs are as defined above. In one embodiment, the ISVD specifically binding to PD-1 comprises or consists of the amino acid sequence of SEQ ID NO: 4170.

In another embodiment, the ISVD specifically binding to human PD-1 may have a sequence identity of more than 90%, such as more than 95% or even more than 99%, with SEQ ID NO: 4171, wherein the CDRs are as defined above. In one embodiment, the ISVD specifically binding to PD-1 comprises or consists of the amino acid sequence of SEQ ID NO: 4171.

In another embodiment, the ISVD specifically binding to human PD-1 may have a sequence identity of more than 90%, such as more than 95% or even more than 99%, with SEQ ID NO: 4172, wherein the CDRs are as defined above. In one embodiment, the ISVD specifically binding to PD-1 comprises or consists of the amino acid sequence of SEQ ID NO: 4172.

In another embodiment, the ISVD specifically binding to human PD-1 may have a sequence identity of more than 90%, such as more than 95% or even more than 99%, with SEQ ID NO: 4173, wherein the CDRs are as defined above. In one embodiment, the ISVD specifically binding to PD-1 comprises or consists of the amino acid sequence of SEQ ID NO: 4173.

In another embodiment, the ISVD specifically binding to human PD-1 may have a sequence identity of more than 90%, such as more than 95% or even more than 99%, with SEQ ID NO: 4174, wherein the CDRs are as defined above. In one embodiment, the ISVD specifically binding to PD-1 comprises or consists of the amino acid sequence of SEQ ID NO: 4174.

In another embodiment, the ISVD specifically binding to human PD-1 may have a sequence identity of more than 90%, such as more than 95% or even more than 99%, with SEQ ID NO: 4175, wherein the CDRs are as defined above. In one embodiment, the ISVD specifically binding to PD-1 comprises or consists of the amino acid sequence of SEQ ID NO: 4175.

In another embodiment, the ISVD specifically binding to human PD-1 may have a sequence identity of more than 90%, such as more than 95% or even more than 99%, with SEQ ID NO: 4176, wherein the CDRs are as defined above. In one embodiment, the ISVD specifically binding to PD-1 comprises or consists of the amino acid sequence of SEQ ID NO: 4176.

In another embodiment, the ISVD specifically binding to human PD-1 may have a sequence identity of more than 90%, such as more than 95% or even more than 99%, with SEQ ID NO: 4177, wherein the CDRs are as defined above. In one embodiment, the ISVD specifically binding to PD-1 comprises or consists of the amino acid sequence of SEQ ID NO: 4177.

In another embodiment, the ISVD specifically binding to human PD-1 may have a sequence identity of more than 90%, such as more than 95% or even more than 99%, with SEQ ID NO: 4178, wherein the CDRs are as defined above. In one embodiment, the ISVD specifically binding to PD-1 comprises or consists of the amino acid sequence of SEQ ID NO: 4178.

In another embodiment, the ISVD specifically binding to human PD-1 may have a sequence identity of more than 90%, such as more than 95% or even more than 99%, with SEQ ID NO: 4179, wherein the CDRs are as defined above. In one embodiment, the ISVD specifically binding to PD-1 comprises or consists of the amino acid sequence of SEQ ID NO: 4179.

In another embodiment, the ISVD specifically binding to human PD-1 may have a sequence identity of more than 90%, such as more than 95% or even more than 99%, with SEQ ID NO: 4180, wherein the CDRs are as defined above. In one embodiment, the ISVD specifically binding to PD-1 comprises or consists of the amino acid sequence of SEQ ID NO: 4180.

In another embodiment, the ISVD specifically binding to human PD-1 may have a sequence identity of more than 90%, such as more than 95% or even more than 99%, with SEQ ID NO: 4181, wherein the CDRs are as defined above. In one embodiment, the ISVD specifically binding to PD-1 comprises or consists of the amino acid sequence of SEQ ID NO: 4181.

In another embodiment, the ISVD specifically binding to human PD-1 may have a sequence identity of more than 90%, such as more than 95% or even more than 99%, with SEQ ID NO: 4182, wherein the CDRs are as defined above. In one embodiment, the ISVD specifically binding to PD-1 comprises or consists of the amino acid sequence of SEQ ID NO: 4182.

In another embodiment, the ISVD specifically binding to human PD-1 may have a sequence identity of more than 90%, such as more than 95% or even more than 99%, with SEQ ID NO: 4183, wherein the CDRs are as defined above. In one embodiment, the ISVD specifically binding to PD-1 comprises or consists of the amino acid sequence of SEQ ID NO: 4183.

In another embodiment, the ISVD specifically binding to human PD-1 may have a sequence identity of more than 90%, such as more than 95% or even more than 99%, with SEQ ID NO: 4184, wherein the CDRs are as defined above. In one embodiment, the ISVD specifically binding to PD-1 comprises or consists of the amino acid sequence of SEQ ID NO: 4184.

In another embodiment, the ISVD specifically binding to human PD-1 may have a sequence identity of more than 90%, such as more than 95% or even more than 99%, with SEQ ID NO: 4185, wherein the CDRs are as defined above. In one embodiment, the ISVD specifically binding to PD-1 comprises or consists of the amino acid sequence of SEQ ID NO: 4185.

In another embodiment, the ISVD specifically binding to human PD-1 may have a sequence identity of more than 90%, such as more than 95% or even more than 99%, with SEQ ID NO: 4186, wherein the CDRs are as defined above. In one embodiment, the ISVD specifically binding to PD-1 comprises or consists of the amino acid sequence of SEQ ID NO: 4186.

In another embodiment, the ISVD specifically binding to human PD-1 may have a sequence identity of more than 90%, such as more than 95% or even more than 99%, with SEQ ID NO: 4187, wherein the CDRs are as defined above. In one embodiment, the ISVD specifically binding to PD-1 comprises or consists of the amino acid sequence of SEQ ID NO: 4187.

In another embodiment, the ISVD specifically binding to human PD-1 may have a sequence identity of more than 90%, such as more than 95% or even more than 99%, with SEQ ID NO: 4188, wherein the CDRs are as defined above. In one embodiment, the ISVD specifically binding to PD-1 comprises or consists of the amino acid sequence of SEQ ID NO: 4188.

In another embodiment, the ISVD specifically binding to human PD-1 may have a sequence identity of more than 90%, such as more than 95% or even more than 99%, with SEQ ID NO: 4189, wherein the CDRs are as defined above. In one embodiment, the ISVD specifically binding to PD-1 comprises or consists of the amino acid sequence of SEQ ID NO: 4189.

In another embodiment, the ISVD specifically binding to human PD-1 may have a sequence identity of more than 90%, such as more than 95% or even more than 99%, with SEQ ID NO: 4190, wherein the CDRs are as defined above. In one embodiment, the ISVD specifically binding to PD-1 comprises or consists of the amino acid sequence of SEQ ID NO: 4190.

In another embodiment, the ISVD specifically binding to human PD-1 may have a sequence identity of more than 90%, such as more than 95% or even more than 99%, with SEQ ID NO: 4191, wherein the CDRs are as defined above. In one embodiment, the ISVD specifically binding to PD-1 comprises or consists of the amino acid sequence of SEQ ID NO: 4191.

In another embodiment, the ISVD specifically binding to human PD-1 may have a sequence identity of more than 90%, such as more than 95% or even more than 99%, with SEQ ID NO: 4192, wherein the CDRs are as defined above. In one embodiment, the ISVD specifically binding to PD-1 comprises or consists of the amino acid sequence of SEQ ID NO: 4192.

In another embodiment, the ISVD specifically binding to human PD-1 may have a sequence identity of more than 90%, such as more than 95% or even more than 99%, with SEQ ID NO: 4193, wherein the CDRs are as defined above. In one embodiment, the ISVD specifically binding to PD-1 comprises or consists of the amino acid sequence of SEQ ID NO: 4193.

In another embodiment, the ISVD specifically binding to human PD-1 may have a sequence identity of more than 90%, such as more than 95% or even more than 99%, with SEQ ID NO: 4194, wherein the CDRs are as defined above. In one embodiment, the ISVD specifically binding to PD-1 comprises or consists of the amino acid sequence of SEQ ID NO: 4194.

In another embodiment, the ISVD specifically binding to human PD-1 may have a sequence identity of more than 90%, such as more than 95% or even more than 99%, with SEQ ID NO: 4195, wherein the CDRs are as defined above. In one embodiment, the ISVD specifically binding to PD-1 comprises or consists of the amino acid sequence of SEQ ID NO: 4195.

In another embodiment, the ISVD specifically binding to human PD-1 may have a sequence identity of more than 90%, such as more than 95% or even more than 99%, with SEQ ID NO: 4196, wherein the CDRs are as defined above. In one embodiment, the ISVD specifically binding to PD-1 comprises or consists of the amino acid sequence of SEQ ID NO: 4196.

In another embodiment, the ISVD specifically binding to human PD-1 may have a sequence identity of more than 90%, such as more than 95% or even more than 99%, with SEQ ID NO: 4197, wherein the CDRs are as defined above. In one embodiment, the ISVD specifically binding to PD-1 comprises or consists of the amino acid sequence of SEQ ID NO: 4197.

In another embodiment, the ISVD specifically binding to human PD-1 may have a sequence identity of more than 90%, such as more than 95% or even more than 99%, with SEQ ID NO: 4198, wherein the CDRs are as defined above. In one embodiment, the ISVD specifically binding to PD-1 comprises or consists of the amino acid sequence of SEQ ID NO: 4198.

In another embodiment, the ISVD specifically binding to human PD-1 may have a sequence identity of more than 90%, such as more than 95% or even more than 99%, with SEQ ID NO: 4199, wherein the CDRs are as defined above. In one embodiment, the ISVD specifically binding to PD-1 comprises or consists of the amino acid sequence of SEQ ID NO: 4199.

In another embodiment, the ISVD specifically binding to human PD-1 may have a sequence identity of more than 90%, such as more than 95% or even more than 99%, with SEQ ID NO: 4200, wherein the CDRs are as defined above. In one embodiment, the ISVD specifically binding to PD-1 comprises or consists of the amino acid sequence of SEQ ID NO: 4200.

In another embodiment, the ISVD specifically binding to human PD-1 may have a sequence identity of more than 90%, such as more than 95% or even more than 99%, with SEQ ID NO: 4201, wherein the CDRs are as defined above. In one embodiment, the ISVD specifically binding to PD-1 comprises or consists of the amino acid sequence of SEQ ID NO: 4201.

In another embodiment, the ISVD specifically binding to human PD-1 may have a sequence identity of more than 90%, such as more than 95% or even more than 99%, with SEQ ID NO: 4202, wherein the CDRs are as defined above. In one embodiment, the ISVD specifically binding to PD-1 comprises or consists of the amino acid sequence of SEQ ID NO: 4202.

In another embodiment, the ISVD specifically binding to human PD-1 may have a sequence identity of more than 90%, such as more than 95% or even more than 99%, with SEQ ID NO: 4203, wherein the CDRs are as defined above. In one embodiment, the ISVD specifically binding to PD-1 comprises or consists of the amino acid sequence of SEQ ID NO: 4203.

In another embodiment, the ISVD specifically binding to human PD-1 may have a sequence identity of more than 90%, such as more than 95% or even more than 99%, with SEQ ID NO: 4204, wherein the CDRs are as defined above. In one embodiment, the ISVD specifically binding to PD-1 comprises or consists of the amino acid sequence of SEQ ID NO: 4204.

In another embodiment, the ISVD specifically binding to human PD-1 may have a sequence identity of more than 90%, such as more than 95% or even more than 99%, with SEQ ID NO: 4205, wherein the CDRs are as defined above. In one embodiment, the ISVD specifically binding to PD-1 comprises or consists of the amino acid sequence of SEQ ID NO: 4205.

In another embodiment, the ISVD specifically binding to human PD-1 may have a sequence identity of more than 90%, such as more than 95% or even more than 99%, with SEQ ID NO: 4206, wherein the CDRs are as defined above. In one embodiment, the ISVD specifically binding to PD-1 comprises or consists of the amino acid sequence of SEQ ID NO: 4206.

In another embodiment, the ISVD specifically binding to human PD-1 may have a sequence identity of more than 90%, such as more than 95% or even more than 99%, with SEQ ID NO: 4207, wherein the CDRs are as defined above. In one embodiment, the ISVD specifically binding to PD-1 comprises or consists of the amino acid sequence of SEQ ID NO: 4207.

In another embodiment, the ISVD specifically binding to human PD-1 may have a sequence identity of more than 90%, such as more than 95% or even more than 99%, with SEQ ID NO: 4208, wherein the CDRs are as defined above. In one embodiment, the ISVD specifically binding to PD-1 comprises or consists of the amino acid sequence of SEQ ID NO: 4208.

In another embodiment, the ISVD specifically binding to human PD-1 may have a sequence identity of more than 90%, such as more than 95% or even more than 99%, with SEQ ID NO: 4209, wherein the CDRs are as defined above. In one embodiment, the ISVD specifically binding to PD-1 comprises or consists of the amino acid sequence of SEQ ID NO: 4209.

In another embodiment, the ISVD specifically binding to human PD-1 may have a sequence identity of more than 90%, such as more than 95% or even more than 99%, with SEQ ID NO: 4210, wherein the CDRs are as defined above. In one embodiment, the ISVD specifically binding to PD-1 comprises or consists of the amino acid sequence of SEQ ID NO: 4210.

In another embodiment, the ISVD specifically binding to human PD-1 may have a sequence identity of more than 90%, such as more than 95% or even more than 99%, with SEQ ID NO: 4211, wherein the CDRs are as defined above. In one embodiment, the ISVD specifically binding to PD-1 comprises or consists of the amino acid sequence of SEQ ID NO: 4211.

In another embodiment, the ISVD specifically binding to human PD-1 may have a sequence identity of more than 90%, such as more than 95% or even more than 99%, with SEQ ID NO: 4212, wherein the CDRs are as defined above. In one embodiment, the ISVD specifically binding to PD-1 comprises or consists of the amino acid sequence of SEQ ID NO: 4212.

In another embodiment, the ISVD specifically binding to human PD-1 may have a sequence identity of more than 90%, such as more than 95% or even more than 99%, with SEQ ID NO: 4213, wherein the CDRs are as defined above. In one embodiment, the ISVD specifically binding to PD-1 comprises or consists of the amino acid sequence of SEQ ID NO: 4213.

In another embodiment, the ISVD specifically binding to human PD-1 may have a sequence identity of more than 90%, such as more than 95% or even more than 99%, with SEQ ID NO: 4214, wherein the CDRs are as defined above. In one embodiment, the ISVD specifically binding to PD-1 comprises or consists of the amino acid sequence of SEQ ID NO: 4214.

In another embodiment, the ISVD specifically binding to human PD-1 may have a sequence identity of more than 90%, such as more than 95% or even more than 99%, with SEQ ID NO: 4215, wherein the CDRs are as defined above. In one embodiment, the ISVD specifically binding to PD-1 comprises or consists of the amino acid sequence of SEQ ID NO: 4215.

In another embodiment, the ISVD specifically binding to human PD-1 may have a sequence identity of more than 90%, such as more than 95% or even more than 99%, with SEQ ID NO: 4216, wherein the CDRs are as defined above. In one embodiment, the ISVD specifically binding to PD-1 comprises or consists of the amino acid sequence of SEQ ID NO: 4216.

In another embodiment, the ISVD specifically binding to human PD-1 may have a sequence identity of more than 90%, such as more than 95% or even more than 99%, with SEQ ID NO: 4217, wherein the CDRs are as defined above. In one embodiment, the ISVD specifically binding to PD-1 comprises or consists of the amino acid sequence of SEQ ID NO: 4217.

In another embodiment, the ISVD specifically binding to human PD-1 may have a sequence identity of more than 90%, such as more than 95% or even more than 99%, with SEQ ID NO: 4218, wherein the CDRs are as defined above. In one embodiment, the ISVD specifically binding to PD-1 comprises or consists of the amino acid sequence of SEQ ID NO: 4218.

In another embodiment, the ISVD specifically binding to human PD-1 may have a sequence identity of more than 90%, such as more than 95% or even more than 99%, with SEQ ID NO: 4219, wherein the CDRs are as defined above. In one embodiment, the ISVD specifically binding to PD-1 comprises or consists of the amino acid sequence of SEQ ID NO: 4219.

In another embodiment, the ISVD specifically binding to human PD-1 may have a sequence identity of more than 90%, such as more than 95% or even more than 99%, with SEQ ID NO: 4220, wherein the CDRs are as defined above. In one embodiment, the ISVD specifically binding to PD-1 comprises or consists of the amino acid sequence of SEQ ID NO: 4220.

In another embodiment, the ISVD specifically binding to human PD-1 may have a sequence identity of more than 90%, such as more than 95% or even more than 99%, with SEQ ID NO: 4221, wherein the CDRs are as defined above. In one embodiment, the ISVD specifically binding to PD-1 comprises or consists of the amino acid sequence of SEQ ID NO: 4221.

In another embodiment, the ISVD specifically binding to human PD-1 may have a sequence identity of more than 90%, such as more than 95% or even more than 99%, with SEQ ID NO: 4222, wherein the CDRs are as defined above. In one embodiment, the ISVD specifically binding to PD-1 comprises or consists of the amino acid sequence of SEQ ID NO: 4222.

In another embodiment, the ISVD specifically binding to human PD-1 may have a sequence identity of more than 90%, such as more than 95% or even more than 99%, with SEQ ID NO: 4223, wherein the CDRs are as defined above. In one embodiment, the ISVD specifically binding to PD-1 comprises or consists of the amino acid sequence of SEQ ID NO: 4223.

In another embodiment, the ISVD specifically binding to human PD-1 may have a sequence identity of more than 90%, such as more than 95% or even more than 99%, with SEQ ID NO: 4224, wherein the CDRs are as defined above. In one embodiment, the ISVD specifically binding to PD-1 comprises or consists of the amino acid sequence of SEQ ID NO: 4224.

In another embodiment, the ISVD specifically binding to human PD-1 may have a sequence identity of more than 90%, such as more than 95% or even more than 99%, with SEQ ID NO: 4225, wherein the CDRs are as defined above. In one embodiment, the ISVD specifically binding to PD-1 comprises or consists of the amino acid sequence of SEQ ID NO: 4225.

In another embodiment, the ISVD specifically binding to human PD-1 may have a sequence identity of more than 90%, such as more than 95% or even more than 99%, with SEQ ID NO: 4226, wherein the CDRs are as defined above. In one embodiment, the ISVD specifically binding to PD-1 comprises or consists of the amino acid sequence of SEQ ID NO: 4226.

In another embodiment, the ISVD specifically binding to human PD-1 may have a sequence identity of more than 90%, such as more than 95% or even more than 99%, with SEQ ID NO: 4227, wherein the CDRs are as defined above. In one embodiment, the ISVD specifically binding to PD-1 comprises or consists of the amino acid sequence of SEQ ID NO: 4227.

In another embodiment, the ISVD specifically binding to human PD-1 may have a sequence identity of more than 90%, such as more than 95% or even more than 99%, with SEQ ID NO: 4228, wherein the CDRs are as defined above. In one embodiment, the ISVD specifically binding to PD-1 comprises or consists of the amino acid sequence of SEQ ID NO: 4228.

In another embodiment, the ISVD specifically binding to human PD-1 may have a sequence identity of more than 90%, such as more than 95% or even more than 99%, with SEQ ID NO: 4229, wherein the CDRs are as defined above. In one embodiment, the ISVD specifically binding to PD-1 comprises or consists of the amino acid sequence of SEQ ID NO: 4229.

In another embodiment, the ISVD specifically binding to human PD-1 may have a sequence identity of more than 90%, such as more than 95% or even more than 99%, with SEQ ID NO: 4230, wherein the CDRs are as defined above. In one embodiment, the ISVD specifically binding to PD-1 comprises or consists of the amino acid sequence of SEQ ID NO: 4230.

In another embodiment, the ISVD specifically binding to human PD-1 may have a sequence identity of more than 90%, such as more than 95% or even more than 99%, with SEQ ID NO: 4231, wherein the CDRs are as defined above. In one embodiment, the ISVD specifically binding to PD-1 comprises or consists of the amino acid sequence of SEQ ID NO: 4231.

In another embodiment, the ISVD specifically binding to human PD-1 may have a sequence identity of more than 90%, such as more than 95% or even more than 99%, with SEQ ID NO: 4232, wherein the CDRs are as defined above. In one embodiment, the ISVD specifically binding to PD-1 comprises or consists of the amino acid sequence of SEQ ID NO: 4232.

In another embodiment, the ISVD specifically binding to human PD-1 may have a sequence identity of more than 90%, such as more than 95% or even more than 99%, with SEQ ID NO: 4233, wherein the CDRs are as defined above. In one embodiment, the ISVD specifically binding to PD-1 comprises or consists of the amino acid sequence of SEQ ID NO: 4233.

In another embodiment, the ISVD specifically binding to human PD-1 may have a sequence identity of more than 90%, such as more than 95% or even more than 99%, with SEQ ID NO: 4234, wherein the CDRs are as defined above. In one embodiment, the ISVD specifically binding to PD-1 comprises or consists of the amino acid sequence of SEQ ID NO: 4234.

In another embodiment, the ISVD specifically binding to human PD-1 may have a sequence identity of more than 90%, such as more than 95% or even more than 99%, with SEQ ID NO: 4235, wherein the CDRs are as defined above. In one embodiment, the ISVD specifically binding to PD-1 comprises or consists of the amino acid sequence of SEQ ID NO: 4235.

In another embodiment, the ISVD specifically binding to human PD-1 may have a sequence identity of more than 90%, such as more than 95% or even more than 99%, with SEQ ID NO: 4236, wherein the CDRs are as defined above. In one embodiment, the ISVD specifically binding to PD-1 comprises or consists of the amino acid sequence of SEQ ID NO: 4236.

In another embodiment, the ISVD specifically binding to human PD-1 may have a sequence identity of more than 90%, such as more than 95% or even more than 99%, with SEQ ID NO: 4237, wherein the CDRs are as defined above. In one embodiment, the ISVD specifically binding to PD-1 comprises or consists of the amino acid sequence of SEQ ID NO: 4237.

In another embodiment, the ISVD specifically binding to human PD-1 may have a sequence identity of more than 90%, such as more than 95% or even more than 99%, with SEQ ID NO: 4238, wherein the CDRs are as defined above. In one embodiment, the ISVD specifically binding to PD-1 comprises or consists of the amino acid sequence of SEQ ID NO: 4238.

In another embodiment, the ISVD specifically binding to human PD-1 may have a sequence identity of more than 90%, such as more than 95% or even more than 99%, with SEQ ID NO: 4239, wherein the CDRs are as defined above. In one embodiment, the ISVD specifically binding to PD-1 comprises or consists of the amino acid sequence of SEQ ID NO: 4239.

In another embodiment, the ISVD specifically binding to human PD-1 may have a sequence identity of more than 90%, such as more than 95% or even more than 99%, with SEQ ID NO: 4240, wherein the CDRs are as defined above. In one embodiment, the ISVD specifically binding to PD-1 comprises or consists of the amino acid sequence of SEQ ID NO: 4240.

In another embodiment, the ISVD specifically binding to human PD-1 may have a sequence identity of more than 90%, such as more than 95% or even more than 99%, with SEQ ID NO: 4241, wherein the CDRs are as defined above. In one embodiment, the ISVD specifically binding to PD-1 comprises or consists of the amino acid sequence of SEQ ID NO: 4241.

In another embodiment, the ISVD specifically binding to human PD-1 may have a sequence identity of more than 90%, such as more than 95% or even more than 99%, with SEQ ID NO: 4242, wherein the CDRs are as defined above. In one embodiment, the ISVD specifically binding to PD-1 comprises or consists of the amino acid sequence of SEQ ID NO: 4242.

In another embodiment, the ISVD specifically binding to human PD-1 may have a sequence identity of more than 90%, such as more than 95% or even more than 99%, with SEQ ID NO: 4243, wherein the CDRs are as defined above. In one embodiment, the ISVD specifically binding to PD-1 comprises or consists of the amino acid sequence of SEQ ID NO: 4243.

In another embodiment, the ISVD specifically binding to human PD-1 may have a sequence identity of more than 90%, such as more than 95% or even more than 99%, with SEQ ID NO: 4244, wherein the CDRs are as defined above. In one embodiment, the ISVD specifically binding to PD-1 comprises or consists of the amino acid sequence of SEQ ID NO: 4244.

In another embodiment, the ISVD specifically binding to human PD-1 may have a sequence identity of more than 90%, such as more than 95% or even more than 99%, with SEQ ID NO: 4245, wherein the CDRs are as defined above. In one embodiment, the ISVD specifically binding to PD-1 comprises or consists of the amino acid sequence of SEQ ID NO: 4245.

In another embodiment, the ISVD specifically binding to human PD-1 may have a sequence identity of more than 90%, such as more than 95% or even more than 99%, with SEQ ID NO: 4246, wherein the CDRs are as defined above. In one embodiment, the ISVD specifically binding to PD-1 comprises or consists of the amino acid sequence of SEQ ID NO: 4246.

In another embodiment, the ISVD specifically binding to human PD-1 may have a sequence identity of more than 90%, such as more than 95% or even more than 99%, with SEQ ID NO: 4247, wherein the CDRs are as defined above. In one embodiment, the ISVD specifically binding to PD-1 comprises or consists of the amino acid sequence of SEQ ID NO: 4247.

In another embodiment, the ISVD specifically binding to human PD-1 may have a sequence identity of more than 90%, such as more than 95% or even more than 99%, with SEQ ID NO: 4248, wherein the CDRs are as defined above. In one embodiment, the ISVD specifically binding to PD-1 comprises or consists of the amino acid sequence of SEQ ID NO: 4248.

In another embodiment, the ISVD specifically binding to human PD-1 may have a sequence identity of more than 90%, such as more than 95% or even more than 99%, with SEQ ID NO: 4249, wherein the CDRs are as defined above. In one embodiment, the ISVD specifically binding to PD-1 comprises or consists of the amino acid sequence of SEQ ID NO: 4249.

In another embodiment, the ISVD specifically binding to human PD-1 may have a sequence identity of more than 90%, such as more than 95% or even more than 99%, with SEQ ID NO: 4250, wherein the CDRs are as defined above. In one embodiment, the ISVD specifically binding to PD-1 comprises or consists of the amino acid sequence of SEQ ID NO: 4250.

In another embodiment, the ISVD specifically binding to human PD-1 may have a sequence identity of more than 90%, such as more than 95% or even more than 99%, with SEQ ID NO: 4251, wherein the CDRs are as defined above. In one embodiment, the ISVD specifically binding to PD-1 comprises or consists of the amino acid sequence of SEQ ID NO: 4251.

In another embodiment, the ISVD specifically binding to human PD-1 may have a sequence identity of more than 90%, such as more than 95% or even more than 99%, with SEQ ID NO: 4252, wherein the CDRs are as defined above. In one embodiment, the ISVD specifically binding to PD-1 comprises or consists of the amino acid sequence of SEQ ID NO: 4252.

In another embodiment, the ISVD specifically binding to human PD-1 may have a sequence identity of more than 90%, such as more than 95% or even more than 99%, with SEQ ID NO: 4253, wherein the CDRs are as defined above. In one embodiment, the ISVD specifically binding to PD-1 comprises or consists of the amino acid sequence of SEQ ID NO: 4253.

In another embodiment, the ISVD specifically binding to human PD-1 may have a sequence identity of more than 90%, such as more than 95% or even more than 99%, with SEQ ID NO: 4254, wherein the CDRs are as defined above. In one embodiment, the ISVD specifically binding to PD-1 comprises or consists of the amino acid sequence of SEQ ID NO: 4254.

In another embodiment, the ISVD specifically binding to human PD-1 may have a sequence identity of more than 90%, such as more than 95% or even more than 99%, with SEQ ID NO: 4255, wherein the CDRs are as defined above. In one embodiment, the ISVD specifically binding to PD-1 comprises or consists of the amino acid sequence of SEQ ID NO: 4255.

In another embodiment, the ISVD specifically binding to human PD-1 may have a sequence identity of more than 90%, such as more than 95% or even more than 99%, with SEQ ID NO: 4256, wherein the CDRs are as defined above. In one embodiment, the ISVD specifically binding to PD-1 comprises or consists of the amino acid sequence of SEQ ID NO: 4256.

In another embodiment, the ISVD specifically binding to human PD-1 may have a sequence identity of more than 90%, such as more than 95% or even more than 99%, with SEQ ID NO: 4257, wherein the CDRs are as defined above. In one embodiment, the ISVD specifically binding to PD-1 comprises or consists of the amino acid sequence of SEQ ID NO: 4257.

In another embodiment, the ISVD specifically binding to human PD-1 may have a sequence identity of more than 90%, such as more than 95% or even more than 99%, with SEQ ID NO: 4258, wherein the CDRs are as defined above. In one embodiment, the ISVD specifically binding to PD-1 comprises or consists of the amino acid sequence of SEQ ID NO: 4258.

In another embodiment, the ISVD specifically binding to human PD-1 may have a sequence identity of more than 90%, such as more than 95% or even more than 99%, with SEQ ID NO: 4259, wherein the CDRs are as defined above. In one embodiment, the ISVD specifically binding to PD-1 comprises or consists of the amino acid sequence of SEQ ID NO: 4259.

In another embodiment, the ISVD specifically binding to human PD-1 may have a sequence identity of more than 90%, such as more than 95% or even more than 99%, with SEQ ID NO: 4260, wherein the CDRs are as defined above. In one embodiment, the ISVD specifically binding to PD-1 comprises or consists of the amino acid sequence of SEQ ID NO: 4260.

In another embodiment, the ISVD specifically binding to human PD-1 may have a sequence identity of more than 90%, such as more than 95% or even more than 99%, with SEQ ID NO: 4261, wherein the CDRs are as defined above. In one embodiment, the ISVD specifically binding to PD-1 comprises or consists of the amino acid sequence of SEQ ID NO: 4261.

In another embodiment, the ISVD specifically binding to human PD-1 may have a sequence identity of more than 90%, such as more than 95% or even more than 99%, with SEQ ID NO: 4262, wherein the CDRs are as defined above. In one embodiment, the ISVD specifically binding to PD-1 comprises or consists of the amino acid sequence of SEQ ID NO: 4262.

In another embodiment, the ISVD specifically binding to human PD-1 may have a sequence identity of more than 90%, such as more than 95% or even more than 99%, with SEQ ID NO: 4263, wherein the CDRs are as defined above. In one embodiment, the ISVD specifically binding to PD-1 comprises or consists of the amino acid sequence of SEQ ID NO: 4263.

In another embodiment, the ISVD specifically binding to human PD-1 may have a sequence identity of more than 90%, such as more than 95% or even more than 99%, with SEQ ID NO: 4264, wherein the CDRs are as defined above. In one embodiment, the ISVD specifically binding to PD-1 comprises or consists of the amino acid sequence of SEQ ID NO: 4264.

In another embodiment, the ISVD specifically binding to human PD-1 may have a sequence identity of more than 90%, such as more than 95% or even more than 99%, with SEQ ID NO: 4265, wherein the CDRs are as defined above. In one embodiment, the ISVD specifically binding to PD-1 comprises or consists of the amino acid sequence of SEQ ID NO: 4265.

In another embodiment, the ISVD specifically binding to human PD-1 may have a sequence identity of more than 90%, such as more than 95% or even more than 99%, with SEQ ID NO: 4266, wherein the CDRs are as defined above. In one embodiment, the ISVD specifically binding to PD-1 comprises or consists of the amino acid sequence of SEQ ID NO: 4266.

In another embodiment, the ISVD specifically binding to human PD-1 may have a sequence identity of more than 90%, such as more than 95% or even more than 99%, with SEQ ID NO: 4267, wherein the CDRs are as defined above. In one embodiment, the ISVD specifically binding to PD-1 comprises or consists of the amino acid sequence of SEQ ID NO: 4267.

In another embodiment, the ISVD specifically binding to human PD-1 may have a sequence identity of more than 90%, such as more than 95% or even more than 99%, with SEQ ID NO: 4268, wherein the CDRs are as defined above. In one embodiment, the ISVD specifically binding to PD-1 comprises or consists of the amino acid sequence of SEQ ID NO: 4268.

In another embodiment, the ISVD specifically binding to human PD-1 may have a sequence identity of more than 90%, such as more than 95% or even more than 99%, with SEQ ID NO: 4269, wherein the CDRs are as defined above. In one embodiment, the ISVD specifically binding to PD-1 comprises or consists of the amino acid sequence of SEQ ID NO: 4269.

In another embodiment, the ISVD specifically binding to human PD-1 may have a sequence identity of more than 90%, such as more than 95% or even more than 99%, with SEQ ID NO: 4270, wherein the CDRs are as defined above. In one embodiment, the ISVD specifically binding to PD-1 comprises or consists of the amino acid sequence of SEQ ID NO: 4270.

In another embodiment, the ISVD specifically binding to human PD-1 may have a sequence identity of more than 90%, such as more than 95% or even more than 99%, with SEQ ID NO: 4271, wherein the CDRs are as defined above. In one embodiment, the ISVD specifically binding to PD-1 comprises or consists of the amino acid sequence of SEQ ID NO: 4271.

In another embodiment, the ISVD specifically binding to human PD-1 may have a sequence identity of more than 90%, such as more than 95% or even more than 99%, with SEQ ID NO: 4272, wherein the CDRs are as defined above. In one embodiment, the ISVD specifically binding to PD-1 comprises or consists of the amino acid sequence of SEQ ID NO: 4272.

In another embodiment, the ISVD specifically binding to human PD-1 may have a sequence identity of more than 90%, such as more than 95% or even more than 99%, with SEQ ID NO: 4273, wherein the CDRs are as defined above. In one embodiment, the ISVD specifically binding to PD-1 comprises or consists of the amino acid sequence of SEQ ID NO: 4273.

In another embodiment, the ISVD specifically binding to human PD-1 may have a sequence identity of more than 90%, such as more than 95% or even more than 99%, with SEQ ID NO: 4274, wherein the CDRs are as defined above. In one embodiment, the ISVD specifically binding to PD-1 comprises or consists of the amino acid sequence of SEQ ID NO: 4274.

In another embodiment, the ISVD specifically binding to human PD-1 may have a sequence identity of more than 90%, such as more than 95% or even more than 99%, with SEQ ID NO: 4275, wherein the CDRs are as defined above. In one embodiment, the ISVD specifically binding to PD-1 comprises or consists of the amino acid sequence of SEQ ID NO: 4275.

In another embodiment, the ISVD specifically binding to human PD-1 may have a sequence identity of more than 90%, such as more than 95% or even more than 99%, with SEQ ID NO: 4276, wherein the CDRs are as defined above. In one embodiment, the ISVD specifically binding to PD-1 comprises or consists of the amino acid sequence of SEQ ID NO: 4276.

In another embodiment, the ISVD specifically binding to human PD-1 may have a sequence identity of more than 90%, such as more than 95% or even more than 99%, with SEQ ID NO: 4277, wherein the CDRs are as defined above. In one embodiment, the ISVD specifically binding to PD-1 comprises or consists of the amino acid sequence of SEQ ID NO: 4277.

In another embodiment, the ISVD specifically binding to human PD-1 may have a sequence identity of more than 90%, such as more than 95% or even more than 99%, with SEQ ID NO: 4278, wherein the CDRs are as defined above. In one embodiment, the ISVD specifically binding to PD-1 comprises or consists of the amino acid sequence of SEQ ID NO: 4278.

In another embodiment, the ISVD specifically binding to human PD-1 may have a sequence identity of more than 90%, such as more than 95% or even more than 99%, with SEQ ID NO: 4279, wherein the CDRs are as defined above. In one embodiment, the ISVD specifically binding to PD-1 comprises or consists of the amino acid sequence of SEQ ID NO: 4279.

In another embodiment, the ISVD specifically binding to human PD-1 may have a sequence identity of more than 90%, such as more than 95% or even more than 99%, with SEQ ID NO: 4280, wherein the CDRs are as defined above. In one embodiment, the ISVD specifically binding to PD-1 comprises or consists of the amino acid sequence of SEQ ID NO: 4280.

In another embodiment, the ISVD specifically binding to human PD-1 may have a sequence identity of more than 90%, such as more than 95% or even more than 99%, with SEQ ID NO: 4281, wherein the CDRs are as defined above. In one embodiment, the ISVD specifically binding to PD-1 comprises or consists of the amino acid sequence of SEQ ID NO: 4281.

In another embodiment, the ISVD specifically binding to human PD-1 may have a sequence identity of more than 90%, such as more than 95% or even more than 99%, with SEQ ID NO: 4282, wherein the CDRs are as defined above. In one embodiment, the ISVD specifically binding to PD-1 comprises or consists of the amino acid sequence of SEQ ID NO: 4282.

In another embodiment, the ISVD specifically binding to human PD-1 may have a sequence identity of more than 90%, such as more than 95% or even more than 99%, with SEQ ID NO: 4283, wherein the CDRs are as defined above. In one embodiment, the ISVD specifically binding to PD-1 comprises or consists of the amino acid sequence of SEQ ID NO: 4283.

In another embodiment, the ISVD specifically binding to human PD-1 may have a sequence identity of more than 90%, such as more than 95% or even more than 99%, with SEQ ID NO: 4284, wherein the CDRs are as defined above. In one embodiment, the ISVD specifically binding to PD-1 comprises or consists of the amino acid sequence of SEQ ID NO: 4284.

In another embodiment, the ISVD specifically binding to human PD-1 may have a sequence identity of more than 90%, such as more than 95% or even more than 99%, with SEQ ID NO: 4285, wherein the CDRs are as defined above. In one embodiment, the ISVD specifically binding to PD-1 comprises or consists of the amino acid sequence of SEQ ID NO: 4285.

In another embodiment, the ISVD specifically binding to human PD-1 may have a sequence identity of more than 90%, such as more than 95% or even more than 99%, with SEQ ID NO: 4286, wherein the CDRs are as defined above. In one embodiment, the ISVD specifically binding to PD-1 comprises or consists of the amino acid sequence of SEQ ID NO: 4286.

In another embodiment, the ISVD specifically binding to human PD-1 may have a sequence identity of more than 90%, such as more than 95% or even more than 99%, with SEQ ID NO: 4287, wherein the CDRs are as defined above. In one embodiment, the ISVD specifically binding to PD-1 comprises or consists of the amino acid sequence of SEQ ID NO: 4287.

In another embodiment, the ISVD specifically binding to human PD-1 may have a sequence identity of more than 90%, such as more than 95% or even more than 99%, with SEQ ID NO: 4288, wherein the CDRs are as defined above. In one embodiment, the ISVD specifically binding to PD-1 comprises or consists of the amino acid sequence of SEQ ID NO: 4288.

In another embodiment, the ISVD specifically binding to human PD-1 may have a sequence identity of more than 90%, such as more than 95% or even more than 99%, with SEQ ID NO: 4289, wherein the CDRs are as defined above. In one embodiment, the ISVD specifically binding to PD-1 comprises or consists of the amino acid sequence of SEQ ID NO: 4289.

In another embodiment, the ISVD specifically binding to human PD-1 may have a sequence identity of more than 90%, such as more than 95% or even more than 99%, with SEQ ID NO: 4290, wherein the CDRs are as defined above. In one embodiment, the ISVD specifically binding to PD-1 comprises or consists of the amino acid sequence of SEQ ID NO: 4290.

In another embodiment, the ISVD specifically binding to human PD-1 may have a sequence identity of more than 90%, such as more than 95% or even more than 99%, with SEQ ID NO: 4291, wherein the CDRs are as defined above. In one embodiment, the ISVD specifically binding to PD-1 comprises or consists of the amino acid sequence of SEQ ID NO: 4291.

In another embodiment, the ISVD specifically binding to human PD-1 may have a sequence identity of more than 90%, such as more than 95% or even more than 99%, with SEQ ID NO: 4292, wherein the CDRs are as defined above. In one embodiment, the ISVD specifically binding to PD-1 comprises or consists of the amino acid sequence of SEQ ID NO: 4292.

In another embodiment, the ISVD specifically binding to human PD-1 may have a sequence identity of more than 90%, such as more than 95% or even more than 99%, with SEQ ID NO: 4293, wherein the CDRs are as defined above. In one embodiment, the ISVD specifically binding to PD-1 comprises or consists of the amino acid sequence of SEQ ID NO: 4293.

In another embodiment, the ISVD specifically binding to human PD-1 may have a sequence identity of more than 90%, such as more than 95% or even more than 99%, with SEQ ID NO: 4294, wherein the CDRs are as defined above. In one embodiment, the ISVD specifically binding to PD-1 comprises or consists of the amino acid sequence of SEQ ID NO: 4294.

In another embodiment, the ISVD specifically binding to human PD-1 may have a sequence identity of more than 90%, such as more than 95% or even more than 99%, with SEQ ID NO: 4295, wherein the CDRs are as defined above. In one embodiment, the ISVD specifically binding to PD-1 comprises or consists of the amino acid sequence of SEQ ID NO: 4295.

In another embodiment, the ISVD specifically binding to human PD-1 may have a sequence identity of more than 90%, such as more than 95% or even more than 99%, with SEQ ID NO: 4296, wherein the CDRs are as defined above. In one embodiment, the ISVD specifically binding to PD-1 comprises or consists of the amino acid sequence of SEQ ID NO: 4296.

In another embodiment, the ISVD specifically binding to human PD-1 may have a sequence identity of more than 90%, such as more than 95% or even more than 99%, with SEQ ID NO: 4297, wherein the CDRs are as defined above. In one embodiment, the ISVD specifically binding to PD-1 comprises or consists of the amino acid sequence of SEQ ID NO: 4297.

In another embodiment, the ISVD specifically binding to human PD-1 may have a sequence identity of more than 90%, such as more than 95% or even more than 99%, with SEQ ID NO: 4298, wherein the CDRs are as defined above. In one embodiment, the ISVD specifically binding to PD-1 comprises or consists of the amino acid sequence of SEQ ID NO: 4298.

In another embodiment, the ISVD specifically binding to human PD-1 may have a sequence identity of more than 90%, such as more than 95% or even more than 99%, with SEQ ID NO: 4299, wherein the CDRs are as defined above. In one embodiment, the ISVD specifically binding to PD-1 comprises or consists of the amino acid sequence of SEQ ID NO: 4299.

In another embodiment, the ISVD specifically binding to human PD-1 may have a sequence identity of more than 90%, such as more than 95% or even more than 99%, with SEQ ID NO: 4300, wherein the CDRs are as defined above. In one embodiment, the ISVD specifically binding to PD-1 comprises or consists of the amino acid sequence of SEQ ID NO: 4300.

In another embodiment, the ISVD specifically binding to human PD-1 may have a sequence identity of more than 90%, such as more than 95% or even more than 99%, with SEQ ID NO: 4301, wherein the CDRs are as defined above. In one embodiment, the ISVD specifically binding to PD-1 comprises or consists of the amino acid sequence of SEQ ID NO: 4301.

In another embodiment, the ISVD specifically binding to human PD-1 may have a sequence identity of more than 90%, such as more than 95% or even more than 99%, with SEQ ID NO: 4302, wherein the CDRs are as defined above. In one embodiment, the ISVD specifically binding to PD-1 comprises or consists of the amino acid sequence of SEQ ID NO: 4302.

In another embodiment, the ISVD specifically binding to human PD-1 may have a sequence identity of more than 90%, such as more than 95% or even more than 99%, with SEQ ID NO: 4303, wherein the CDRs are as defined above. In one embodiment, the ISVD specifically binding to PD-1 comprises or consists of the amino acid sequence of SEQ ID NO: 4303.

In another embodiment, the ISVD specifically binding to human PD-1 may have a sequence identity of more than 90%, such as more than 95% or even more than 99%, with SEQ ID NO: 4304, wherein the CDRs are as defined above. In one embodiment, the ISVD specifically binding to PD-1 comprises or consists of the amino acid sequence of SEQ ID NO: 4304.

In another embodiment, the ISVD specifically binding to human PD-1 may have a sequence identity of more than 90%, such as more than 95% or even more than 99%, with SEQ ID NO: 4305, wherein the CDRs are as defined above. In one embodiment, the ISVD specifically binding to PD-1 comprises or consists of the amino acid sequence of SEQ ID NO: 4305.

In another embodiment, the ISVD specifically binding to human PD-1 may have a sequence identity of more than 90%, such as more than 95% or even more than 99%, with SEQ ID NO: 4306, wherein the CDRs are as defined above. In one embodiment, the ISVD specifically binding to PD-1 comprises or consists of the amino acid sequence of SEQ ID NO: 4306.

In another embodiment, the ISVD specifically binding to human PD-1 may have a sequence identity of more than 90%, such as more than 95% or even more than 99%, with SEQ ID NO: 4307, wherein the CDRs are as defined above. In one embodiment, the ISVD specifically binding to PD-1 comprises or consists of the amino acid sequence of SEQ ID NO: 4307.

In another embodiment, the ISVD specifically binding to human PD-1 may have a sequence identity of more than 90%, such as more than 95% or even more than 99%, with SEQ ID NO: 4308, wherein the CDRs are as defined above. In one embodiment, the ISVD specifically binding to PD-1 comprises or consists of the amino acid sequence of SEQ ID NO: 4308.

In another embodiment, the ISVD specifically binding to human PD-1 may have a sequence identity of more than 90%, such as more than 95% or even more than 99%, with SEQ ID NO: 4309, wherein the CDRs are as defined above. In one embodiment, the ISVD specifically binding to PD-1 comprises or consists of the amino acid sequence of SEQ ID NO: 4309.

In another embodiment, the ISVD specifically binding to human PD-1 may have a sequence identity of more than 90%, such as more than 95% or even more than 99%, with SEQ ID NO: 4310, wherein the CDRs are as defined above. In one embodiment, the ISVD specifically binding to PD-1 comprises or consists of the amino acid sequence of SEQ ID NO: 4310.

In another embodiment, the ISVD specifically binding to human PD-1 may have a sequence identity of more than 90%, such as more than 95% or even more than 99%, with SEQ ID NO: 4311, wherein the CDRs are as defined above. In one embodiment, the ISVD specifically binding to PD-1 comprises or consists of the amino acid sequence of SEQ ID NO: 4311.

In another embodiment, the ISVD specifically binding to human PD-1 may have a sequence identity of more than 90%, such as more than 95% or even more than 99%, with SEQ ID NO: 4312, wherein the CDRs are as defined above. In one embodiment, the ISVD specifically binding to PD-1 comprises or consists of the amino acid sequence of SEQ ID NO: 4312.

In another embodiment, the ISVD specifically binding to human PD-1 may have a sequence identity of more than 90%, such as more than 95% or even more than 99%, with SEQ ID NO: 4313, wherein the CDRs are as defined above. In one embodiment, the ISVD specifically binding to PD-1 comprises or consists of the amino acid sequence of SEQ ID NO: 4313.

In another embodiment, the ISVD specifically binding to human PD-1 may have a sequence identity of more than 90%, such as more than 95% or even more than 99%, with SEQ ID NO: 4314, wherein the CDRs are as defined above. In one embodiment, the ISVD specifically binding to PD-1 comprises or consists of the amino acid sequence of SEQ ID NO: 4314.

In another embodiment, the ISVD specifically binding to human PD-1 may have a sequence identity of more than 90%, such as more than 95% or even more than 99%, with SEQ ID NO: 4315, wherein the CDRs are as defined above. In one embodiment, the ISVD specifically binding to PD-1 comprises or consists of the amino acid sequence of SEQ ID NO: 4315.

In another embodiment, the ISVD specifically binding to human PD-1 may have a sequence identity of more than 90%, such as more than 95% or even more than 99%, with SEQ ID NO: 4316, wherein the CDRs are as defined above. In one embodiment, the ISVD specifically binding to PD-1 comprises or consists of the amino acid sequence of SEQ ID NO: 4316.

In another embodiment, the ISVD specifically binding to human PD-1 may have a sequence identity of more than 90%, such as more than 95% or even more than 99%, with SEQ ID NO: 4317, wherein the CDRs are as defined above. In one embodiment, the ISVD specifically binding to PD-1 comprises or consists of the amino acid sequence of SEQ ID NO: 4317.

In another embodiment, the ISVD specifically binding to human PD-1 may have a sequence identity of more than 90%, such as more than 95% or even more than 99%, with SEQ ID NO: 4318, wherein the CDRs are as defined above. In one embodiment, the ISVD specifically binding to PD-1 comprises or consists of the amino acid sequence of SEQ ID NO: 4318.

In another embodiment, the ISVD specifically binding to human PD-1 may have a sequence identity of more than 90%, such as more than 95% or even more than 99%, with SEQ ID NO: 4319, wherein the CDRs are as defined above. In one embodiment, the ISVD specifically binding to PD-1 comprises or consists of the amino acid sequence of SEQ ID NO: 4319.

In another embodiment, the ISVD specifically binding to human PD-1 may have a sequence identity of more than 90%, such as more than 95% or even more than 99%, with SEQ ID NO: 4320, wherein the CDRs are as defined above. In one embodiment, the ISVD specifically binding to PD-1 comprises or consists of the amino acid sequence of SEQ ID NO: 4320.

In another embodiment, the ISVD specifically binding to human PD-1 may have a sequence identity of more than 90%, such as more than 95% or even more than 99%, with SEQ ID NO: 4321, wherein the CDRs are as defined above. In one embodiment, the ISVD specifically binding to PD-1 comprises or consists of the amino acid sequence of SEQ ID NO: 4321.

In another embodiment, the ISVD specifically binding to human PD-1 may have a sequence identity of more than 90%, such as more than 95% or even more than 99%, with SEQ ID NO: 4322, wherein the CDRs are as defined above. In one embodiment, the ISVD specifically binding to PD-1 comprises or consists of the amino acid sequence of SEQ ID NO: 4322.

In another embodiment, the ISVD specifically binding to human PD-1 may have a sequence identity of more than 90%, such as more than 95% or even more than 99%, with SEQ ID NO: 4323, wherein the CDRs are as defined above. In one embodiment, the ISVD specifically binding to PD-1 comprises or consists of the amino acid sequence of SEQ ID NO: 4323.

In another embodiment, the ISVD specifically binding to human PD-1 may have a sequence identity of more than 90%, such as more than 95% or even more than 99%, with SEQ ID NO: 4324, wherein the CDRs are as defined above. In one embodiment, the ISVD specifically binding to PD-1 comprises or consists of the amino acid sequence of SEQ ID NO: 4324.

In another embodiment, the ISVD specifically binding to human PD-1 may have a sequence identity of more than 90%, such as more than 95% or even more than 99%, with SEQ ID NO: 4325, wherein the CDRs are as defined above. In one embodiment, the ISVD specifically binding to PD-1 comprises or consists of the amino acid sequence of SEQ ID NO: 4325.

In another embodiment, the ISVD specifically binding to human PD-1 may have a sequence identity of more than 90%, such as more than 95% or even more than 99%, with SEQ ID NO: 4326, wherein the CDRs are as defined above. In one embodiment, the ISVD specifically binding to PD-1 comprises or consists of the amino acid sequence of SEQ ID NO: 4326.

In another embodiment, the ISVD specifically binding to human PD-1 may have a sequence identity of more than 90%, such as more than 95% or even more than 99%, with SEQ ID NO: 4327, wherein the CDRs are as defined above. In one embodiment, the ISVD specifically binding to PD-1 comprises or consists of the amino acid sequence of SEQ ID NO: 4327.

In another embodiment, the ISVD specifically binding to human PD-1 may have a sequence identity of more than 90%, such as more than 95% or even more than 99%, with SEQ ID NO: 4328, wherein the CDRs are as defined above. In one embodiment, the ISVD specifically binding to PD-1 comprises or consists of the amino acid sequence of SEQ ID NO: 4328.

In another embodiment, the ISVD specifically binding to human PD-1 may have a sequence identity of more than 90%, such as more than 95% or even more than 99%, with SEQ ID NO: 4329, wherein the CDRs are as defined above. In one embodiment, the ISVD specifically binding to PD-1 comprises or consists of the amino acid sequence of SEQ ID NO: 4329.

In another embodiment, the ISVD specifically binding to human PD-1 may have a sequence identity of more than 90%, such as more than 95% or even more than 99%, with SEQ ID NO: 4330, wherein the CDRs are as defined above. In one embodiment, the ISVD specifically binding to PD-1 comprises or consists of the amino acid sequence of SEQ ID NO: 4330.

In another embodiment, the ISVD specifically binding to human PD-1 may have a sequence identity of more than 90%, such as more than 95% or even more than 99%, with SEQ ID NO: 4331, wherein the CDRs are as defined above. In one embodiment, the ISVD specifically binding to PD-1 comprises or consists of the amino acid sequence of SEQ ID NO: 4331.

In another embodiment, the ISVD specifically binding to human PD-1 may have a sequence identity of more than 90%, such as more than 95% or even more than 99%, with SEQ ID NO: 4332, wherein the CDRs are as defined above. In one embodiment, the ISVD specifically binding to PD-1 comprises or consists of the amino acid sequence of SEQ ID NO: 4332.

In another embodiment, the ISVD specifically binding to human PD-1 may have a sequence identity of more than 90%, such as more than 95% or even more than 99%, with SEQ ID NO: 4333, wherein the CDRs are as defined above. In one embodiment, the ISVD specifically binding to PD-1 comprises or consists of the amino acid sequence of SEQ ID NO: 4333.

In another embodiment, the ISVD specifically binding to human PD-1 may have a sequence identity of more than 90%, such as more than 95% or even more than 99%, with SEQ ID NO: 4334, wherein the CDRs are as defined above. In one embodiment, the ISVD specifically binding to PD-1 comprises or consists of the amino acid sequence of SEQ ID NO: 4334.

In another embodiment, the ISVD specifically binding to human PD-1 may have a sequence identity of more than 90%, such as more than 95% or even more than 99%, with SEQ ID NO: 4335, wherein the CDRs are as defined above. In one embodiment, the ISVD specifically binding to PD-1 comprises or consists of the amino acid sequence of SEQ ID NO: 4335.

In another embodiment, the ISVD specifically binding to human PD-1 may have a sequence identity of more than 90%, such as more than 95% or even more than 99%, with SEQ ID NO: 4336, wherein the CDRs are as defined above. In one embodiment, the ISVD specifically binding to PD-1 comprises or consists of the amino acid sequence of SEQ ID NO: 4336.

In another embodiment, the ISVD specifically binding to human PD-1 may have a sequence identity of more than 90%, such as more than 95% or even more than 99%, with SEQ ID NO: 4337, wherein the CDRs are as defined above. In one embodiment, the ISVD specifically binding to PD-1 comprises or consists of the amino acid sequence of SEQ ID NO: 4337.

In another embodiment, the ISVD specifically binding to human PD-1 may have a sequence identity of more than 90%, such as more than 95% or even more than 99%, with SEQ ID NO: 4338, wherein the CDRs are as defined above. In one embodiment, the ISVD specifically binding to PD-1 comprises or consists of the amino acid sequence of SEQ ID NO: 4338.

In another embodiment, the ISVD specifically binding to human PD-1 may have a sequence identity of more than 90%, such as more than 95% or even more than 99%, with SEQ ID NO: 4339, wherein the CDRs are as defined above. In one embodiment, the ISVD specifically binding to PD-1 comprises or consists of the amino acid sequence of SEQ ID NO: 4339.

In another embodiment, the ISVD specifically binding to human PD-1 may have a sequence identity of more than 90%, such as more than 95% or even more than 99%, with SEQ ID NO: 4340, wherein the CDRs are as defined above. In one embodiment, the ISVD specifically binding to PD-1 comprises or consists of the amino acid sequence of SEQ ID NO: 4340.

In another embodiment, the ISVD specifically binding to human PD-1 may have a sequence identity of more than 90%, such as more than 95% or even more than 99%, with SEQ ID NO: 4341, wherein the CDRs are as defined above. In one embodiment, the ISVD specifically binding to PD-1 comprises or consists of the amino acid sequence of SEQ ID NO: 4341.

In another embodiment, the ISVD specifically binding to human PD-1 may have a sequence identity of more than 90%, such as more than 95% or even more than 99%, with SEQ ID NO: 4342, wherein the CDRs are as defined above. In one embodiment, the ISVD specifically binding to PD-1 comprises or consists of the amino acid sequence of SEQ ID NO: 4342.

In another embodiment, the ISVD specifically binding to human PD-1 may have a sequence identity of more than 90%, such as more than 95% or even more than 99%, with SEQ ID NO: 4343, wherein the CDRs are as defined above. In one embodiment, the ISVD specifically binding to PD-1 comprises or consists of the amino acid sequence of SEQ ID NO: 4343.

In another embodiment, the ISVD specifically binding to human PD-1 may have a sequence identity of more than 90%, such as more than 95% or even more than 99%, with SEQ ID NO: 4344, wherein the CDRs are as defined above. In one embodiment, the ISVD specifically binding to PD-1 comprises or consists of the amino acid sequence of SEQ ID NO: 4344.

In another embodiment, the ISVD specifically binding to human PD-1 may have a sequence identity of more than 90%, such as more than 95% or even more than 99%, with SEQ ID NO: 4345, wherein the CDRs are as defined above. In one embodiment, the ISVD specifically binding to PD-1 comprises or consists of the amino acid sequence of SEQ ID NO: 4345.

In another embodiment, the ISVD specifically binding to human PD-1 may have a sequence identity of more than 90%, such as more than 95% or even more than 99%, with SEQ ID NO: 4346, wherein the CDRs are as defined above. In one embodiment, the ISVD specifically binding to PD-1 comprises or consists of the amino acid sequence of SEQ ID NO: 4346.

In another embodiment, the ISVD specifically binding to human PD-1 may have a sequence identity of more than 90%, such as more than 95% or even more than 99%, with SEQ ID NO: 4347, wherein the CDRs are as defined above. In one embodiment, the ISVD specifically binding to PD-1 comprises or consists of the amino acid sequence of SEQ ID NO: 4347.

In another embodiment, the ISVD specifically binding to human PD-1 may have a sequence identity of more than 90%, such as more than 95% or even more than 99%, with SEQ ID NO: 4348, wherein the CDRs are as defined above. In one embodiment, the ISVD specifically binding to PD-1 comprises or consists of the amino acid sequence of SEQ ID NO: 4348.

In another embodiment, the ISVD specifically binding to human PD-1 may have a sequence identity of more than 90%, such as more than 95% or even more than 99%, with SEQ ID NO: 4349, wherein the CDRs are as defined above. In one embodiment, the ISVD specifically binding to PD-1 comprises or consists of the amino acid sequence of SEQ ID NO: 4349.

In another embodiment, the ISVD specifically binding to human PD-1 may have a sequence identity of more than 90%, such as more than 95% or even more than 99%, with SEQ ID NO: 4350, wherein the CDRs are as defined above. In one embodiment, the ISVD specifically binding to PD-1 comprises or consists of the amino acid sequence of SEQ ID NO: 4350.

In another embodiment, the ISVD specifically binding to human PD-1 may have a sequence identity of more than 90%, such as more than 95% or even more than 99%, with SEQ ID NO: 4351, wherein the CDRs are as defined above. In one embodiment, the ISVD specifically binding to PD-1 comprises or consists of the amino acid sequence of SEQ ID NO: 4351.

In another embodiment, the ISVD specifically binding to human PD-1 may have a sequence identity of more than 90%, such as more than 95% or even more than 99%, with SEQ ID NO: 4352, wherein the CDRs are as defined above. In one embodiment, the ISVD specifically binding to PD-1 comprises or consists of the amino acid sequence of SEQ ID NO: 4352.

In another embodiment, the ISVD specifically binding to human PD-1 may have a sequence identity of more than 90%, such as more than 95% or even more than 99%, with SEQ ID NO: 4353, wherein the CDRs are as defined above. In one embodiment, the ISVD specifically binding to PD-1 comprises or consists of the amino acid sequence of SEQ ID NO: 4353.

In another embodiment, the ISVD specifically binding to human PD-1 may have a sequence identity of more than 90%, such as more than 95% or even more than 99%, with SEQ ID NO: 4354, wherein the CDRs are as defined above. In one embodiment, the ISVD specifically binding to PD-1 comprises or consists of the amino acid sequence of SEQ ID NO: 4354.

In another embodiment, the ISVD specifically binding to human PD-1 may have a sequence identity of more than 90%, such as more than 95% or even more than 99%, with SEQ ID NO: 4355, wherein the CDRs are as defined above. In one embodiment, the ISVD specifically binding to PD-1 comprises or consists of the amino acid sequence of SEQ ID NO: 4355.

In another embodiment, the ISVD specifically binding to human PD-1 may have a sequence identity of more than 90%, such as more than 95% or even more than 99%, with SEQ ID NO: 4356, wherein the CDRs are as defined above. In one embodiment, the ISVD specifically binding to PD-1 comprises or consists of the amino acid sequence of SEQ ID NO: 4356.

In another embodiment, the ISVD specifically binding to human PD-1 may have a sequence identity of more than 90%, such as more than 95% or even more than 99%, with SEQ ID NO: 4357, wherein the CDRs are as defined above. In one embodiment, the ISVD specifically binding to PD-1 comprises or consists of the amino acid sequence of SEQ ID NO: 4357.

In another embodiment, the ISVD specifically binding to human PD-1 may have a sequence identity of more than 90%, such as more than 95% or even more than 99%, with SEQ ID NO: 4358, wherein the CDRs are as defined above. In one embodiment, the ISVD specifically binding to PD-1 comprises or consists of the amino acid sequence of SEQ ID NO: 4358.

In another embodiment, the ISVD specifically binding to human PD-1 may have a sequence identity of more than 90%, such as more than 95% or even more than 99%, with SEQ ID NO: 4359, wherein the CDRs are as defined above. In one embodiment, the ISVD specifically binding to PD-1 comprises or consists of the amino acid sequence of SEQ ID NO: 4359.

In another embodiment, the ISVD specifically binding to human PD-1 may have a sequence identity of more than 90%, such as more than 95% or even more than 99%, with SEQ ID NO: 4360, wherein the CDRs are as defined above. In one embodiment, the ISVD specifically binding to PD-1 comprises or consists of the amino acid sequence of SEQ ID NO: 4360.

In another embodiment, the ISVD specifically binding to human PD-1 may have a sequence identity of more than 90%, such as more than 95% or even more than 99%, with SEQ ID NO: 4361, wherein the CDRs are as defined above. In one embodiment, the ISVD specifically binding to PD-1 comprises or consists of the amino acid sequence of SEQ ID NO: 4361.

In another embodiment, the ISVD specifically binding to human PD-1 may have a sequence identity of more than 90%, such as more than 95% or even more than 99%, with SEQ ID NO: 4362, wherein the CDRs are as defined above. In one embodiment, the ISVD specifically binding to PD-1 comprises or consists of the amino acid sequence of SEQ ID NO: 4362.

In another embodiment, the ISVD specifically binding to human PD-1 may have a sequence identity of more than 90%, such as more than 95% or even more than 99%, with SEQ ID NO: 4363, wherein the CDRs are as defined above. In one embodiment, the ISVD specifically binding to PD-1 comprises or consists of the amino acid sequence of SEQ ID NO: 4363.

In another embodiment, the ISVD specifically binding to human PD-1 may have a sequence identity of more than 90%, such as more than 95% or even more than 99%, with SEQ ID NO: 4364, wherein the CDRs are as defined above. In one embodiment, the ISVD specifically binding to PD-1 comprises or consists of the amino acid sequence of SEQ ID NO: 4364.

In another embodiment, the ISVD specifically binding to human PD-1 may have a sequence identity of more than 90%, such as more than 95% or even more than 99%, with SEQ ID NO: 4365, wherein the CDRs are as defined above. In one embodiment, the ISVD specifically binding to PD-1 comprises or consists of the amino acid sequence of SEQ ID NO: 4365.

In another embodiment, the ISVD specifically binding to human PD-1 may have a sequence identity of more than 90%, such as more than 95% or even more than 99%, with SEQ ID NO: 4366, wherein the CDRs are as defined above. In one embodiment, the ISVD specifically binding to PD-1 comprises or consists of the amino acid sequence of SEQ ID NO: 4366.

In another embodiment, the ISVD specifically binding to human PD-1 may have a sequence identity of more than 90%, such as more than 95% or even more than 99%, with SEQ ID NO: 4367, wherein the CDRs are as defined above. In one embodiment, the ISVD specifically binding to PD-1 comprises or consists of the amino acid sequence of SEQ ID NO: 4367.

In another embodiment, the ISVD specifically binding to human PD-1 may have a sequence identity of more than 90%, such as more than 95% or even more than 99%, with SEQ ID NO: 4368, wherein the CDRs are as defined above. In one embodiment, the ISVD specifically binding to PD-1 comprises or consists of the amino acid sequence of SEQ ID NO: 4368.

In another embodiment, the ISVD specifically binding to human PD-1 may have a sequence identity of more than 90%, such as more than 95% or even more than 99%, with SEQ ID NO: 4369, wherein the CDRs are as defined above. In one embodiment, the ISVD specifically binding to PD-1 comprises or consists of the amino acid sequence of SEQ ID NO: 4369.

In another embodiment, the ISVD specifically binding to human PD-1 may have a sequence identity of more than 90%, such as more than 95% or even more than 99%, with SEQ ID NO: 4370, wherein the CDRs are as defined above. In one embodiment, the ISVD specifically binding to PD-1 comprises or consists of the amino acid sequence of SEQ ID NO: 4370.

In another embodiment, the ISVD specifically binding to human PD-1 may have a sequence identity of more than 90%, such as more than 95% or even more than 99%, with SEQ ID NO: 4371, wherein the CDRs are as defined above. In one embodiment, the ISVD specifically binding to PD-1 comprises or consists of the amino acid sequence of SEQ ID NO: 4371.

In another embodiment, the ISVD specifically binding to human PD-1 may have a sequence identity of more than 90%, such as more than 95% or even more than 99%, with SEQ ID NO: 4372, wherein the CDRs are as defined above. In one embodiment, the ISVD specifically binding to PD-1 comprises or consists of the amino acid sequence of SEQ ID NO: 4372.

In another embodiment, the ISVD specifically binding to human PD-1 may have a sequence identity of more than 90%, such as more than 95% or even more than 99%, with SEQ ID NO: 4373, wherein the CDRs are as defined above. In one embodiment, the ISVD specifically binding to PD-1 comprises or consists of the amino acid sequence of SEQ ID NO: 4373.

In another embodiment, the ISVD specifically binding to human PD-1 may have a sequence identity of more than 90%, such as more than 95% or even more than 99%, with SEQ ID NO: 4374, wherein the CDRs are as defined above. In one embodiment, the ISVD specifically binding to PD-1 comprises or consists of the amino acid sequence of SEQ ID NO: 4374.

In another embodiment, the ISVD specifically binding to human PD-1 may have a sequence identity of more than 90%, such as more than 95% or even more than 99%, with SEQ ID NO: 4375, wherein the CDRs are as defined above. In one embodiment, the ISVD specifically binding to PD-1 comprises or consists of the amino acid sequence of SEQ ID NO: 4375.

In another embodiment, the ISVD specifically binding to human PD-1 may have a sequence identity of more than 90%, such as more than 95% or even more than 99%, with SEQ ID NO: 4376, wherein the CDRs are as defined above. In one embodiment, the ISVD specifically binding to PD-1 comprises or consists of the amino acid sequence of SEQ ID NO: 4376.

In another embodiment, the ISVD specifically binding to human PD-1 may have a sequence identity of more than 90%, such as more than 95% or even more than 99%, with SEQ ID NO: 4377, wherein the CDRs are as defined above. In one embodiment, the ISVD specifically binding to PD-1 comprises or consists of the amino acid sequence of SEQ ID NO: 4377.

In another embodiment, the ISVD specifically binding to human PD-1 may have a sequence identity of more than 90%, such as more than 95% or even more than 99%, with SEQ ID NO: 4378, wherein the CDRs are as defined above. In one embodiment, the ISVD specifically binding to PD-1 comprises or consists of the amino acid sequence of SEQ ID NO: 4378.

Specific examples of such ISVDs that specifically bind to PD-1 have one or more, or all, framework regions as indicated for A0294040C12 and sequence optimized variants thereof in Table A-5 and Table A-10 (in addition to the CDRs as defined above). In one embodiment, the ISVD comprises or consists of the full amino acid sequence of A0294040C12 (SEQ ID NO: 4056), A029403745 (SEQ ID NO: 4094), A029404661 (SEQ ID NO: 4183), or A029404809 (SEQ ID NO: 4210) (see Table A-5 and A10)

The SEQ ID NOs for the CDR sequences referred to above are based on the CDR definition according to the AbM definition (see Table A-10). It is noted that the SEQ ID NOs for the CDR sequences defined according to the Kabat definition, Chothia definition or IMGT definition can likewise be used.

Accordingly, the ISVDs provided by the present technology, specifically binding to PD-1 as described above using the AbM definition, can be also described using the Kabat definition, Chothia definition or IMGT definition.

In one embodiment, the ISVD comprises CDRs (Kabat definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 4049-4378.

In one embodiment, the ISVD comprises CDRs (Kabat definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 4056, 4094, 4183, and 4210.

In one embodiment, the ISVD comprises CDRs (Chothia definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 4049-4378, preferably selected from SEQ ID NOs: 4056, 4094, 4183, and 4210.

In one embodiment, the ISVD comprises CDRs (IMGT definition) with an amino acid sequence that has at least 70% amino acid sequence identity, preferably at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 4049-4378, preferably selected from SEQ ID NOs: 4056, 4094, 4183, and 4210.

In another embodiment, when such an ISVD specifically binding to PD-1 has 3, 2 or 1 amino acid difference in at least one CDR relative to a corresponding reference CDR sequence (above), the ISVD has at least (essentially) the same binding affinity to human PD-1 compared to one of the VHHs with SEQ ID NOs: 4056, 4094, 4183, or 4210, wherein the binding affinity is measured using the same method, such as e.g. SPR.

In another embodiment, when such an ISVD specifically binding to PD-1 has 3, 2 or 1 amino acid difference in at least one CDR relative to a corresponding reference CDR sequence (above), the ISVD has at least (essentially) the same binding affinity to cyno PD-1 compared to one of the VHHs with SEQ ID NOs: 4056, 4094, 4183, or 4210, wherein the binding affinity is measured using the same method, such as e.g. SPR.

In another embodiment, when such an ISVD specifically binding to PD-1 has 3, 2 or 1 amino acid difference in at least one CDR relative to a corresponding reference CDR sequence (above), the ISVD still specifically binds to human PD-1 and cyno PD-1 with less than 10-fold difference in affinity (KD).

In another embodiment, when such an ISVD specifically binding to PD-1 has 3, 2 or 1 amino acid difference in at least one CDR relative to a corresponding reference CDR sequence (above), the ISVD still specifically binds to human and cyno PD-1 and does not bind to other members of the CD28 receptor family.

In another embodiment, when such an ISVD specifically binding to PD-1 has 3, 2 or 1 amino acid difference in at least one CDR relative to a corresponding reference CDR sequence (above), the ISVD still specifically binds to human and cyno PD-1 and does not bind to CD28, CTLA-4, ICOS, and BTLA.

In another embodiment, when such an ISVD specifically binding to PD-1 has 3, 2 or 1 amino acid difference in at least one CDR relative to a corresponding reference CDR sequence (above), the ISVD blocks the interaction of human PD-1 with human PD-L1 with at least (essentially) the same potency compared to one of the VHHs with SEQ ID NOs: 4056, 4094, 4183, or 4210, wherein the potency is measured, e.g., in FACS competition assay on CHO cells expressing human PD-1, with hPD-L1 as competitor.

In another embodiment, when such an ISVD specifically binding to PD-1 has 3, 2 or 1 amino acid difference in at least one CDR relative to a corresponding reference CDR sequence (above), the ISVD blocks the interaction of cyno PD-1 with cyno PD-L1 with at least (essentially) the same potency compared to one of the VHHs with SEQ ID NOs: 4056, 4094, 4183, or 4210, wherein the potency is measured, e.g., in FACS competition assay on CHO cells expressing cyno PD-1, with hPD-L1 as competitor.

In another embodiment, when such an ISVD specifically binding to PD-1 has 3, 2 or 1 amino acid difference in at least one CDR relative to a corresponding reference CDR sequence (above), the ISVD blocks the interaction of hPD-1 with PD-L1 with (essentially) the same % inhibition or more compared to one of the VHHs with SEQ ID NOs: 4056, 4094, 4183, or 4210, wherein the % inhibition is determined, e.g., in a FACS competition assay on CHO cells expressing human PD-1, with hPD-L1 as competitor.

In another embodiment, when such an ISVD specifically binding to PD-1 has 3, 2 or 1 amino acid difference in at least one CDR relative to a corresponding reference CDR sequence (above), the ISVD has at least (essentially) the same potency for T-cell activation compared to one of the VHHs with SEQ ID NOs: 4056, 4094, 4183, or 4210, wherein the potency is monitored, e.g., via IFN-gamma production in an autologous Tetanus Toxoid recall assay (TT assay), e.g. the TT assay as described herein.

In another embodiment, when such an ISVD specifically binding to PD-1 has 3, 2 or 1 amino acid difference in at least one CDR relative to a corresponding reference CDR sequence (above), the ISVD has at least (essentially) the same efficacy for T-cell activation compared to one of the VHHs with SEQ ID NOs: 4056, 4094, 4183, or 4210, wherein the efficacy is monitored, e.g., via IFN-gamma production in an autologous Tetanus Toxoid recall assay (TT assay), e.g. the TT assay as described herein

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same binding affinity to human PD-1 compared to one of the VHHs with SEQ ID NOs: 4056, 4094, 4183, or 4210, wherein the binding affinity is measured using the same method, such as e.g. SPR.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same binding affinity to cyno PD-1 compared to one of the VHHs with SEQ ID NOs: 4056, 4094, 4183, or 4210, wherein the binding affinity is measured using the same method, such as e.g. SPR.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human PD-1 and cyno PD-1 with less than 10-fold difference in affinity (KD).

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human and cyno PD-1 and does not bind to other members of the CD28 receptor family.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD still specifically binds to human and cyno PD-1 and does not bind to CD28, CTLA-4, ICOS, and BTLA.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of human PD-1 with human PD-L1 with at least (essentially) the same potency compared to one of the VHHs with SEQ ID NOs: 4056, 4094, 4183, or 4210, wherein the potency is measured, e.g., in FACS competition assay on CHO cells expressing human PD-1, with hPD-L1 as competitor.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of cyno PD-1 with cyno PD-L1 with at least (essentially) the same potency compared to one of the VHHs with SEQ ID NOs: 4056, 4094, 4183, or 4210, wherein the potency is measured, e.g., in FACS competition assay on CHO cells expressing cyno PD-1, with hPD-L1 as competitor

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD blocks the interaction of hPD-1 with (h) PD-L1 with (essentially) the same % inhibition or more compared to one of the VHHs with SEQ ID NOs: 4056, 4094, 4183, or 4210, wherein the % inhibition is determined, e.g., in a FACS competition assay.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same potency for efficacy for T-cell activation compared to one of the VHHs with SEQ ID NOs: 4056, 4094, 4183, or 4210, wherein the potency is monitored, e.g., via IFN-gamma production in an autologous Tetanus Toxoid recall assay (TT assay), e.g. the TT assay as described herein.

In another embodiment, when the CDRs of such an ISVD specifically binding to PD-1 have least 90% amino acid sequence identity, at least 95% amino acid sequence identity, such as 99% amino acid sequence identity or more, with a corresponding reference CDR sequence (above), the ISVD has at least (essentially) the same efficacy for T-cell activation compared to one of the VHHs with SEQ ID NOs: 4056, 4094, 4183, or 4210, wherein the efficacy is monitored, e.g., via IFN-gamma production in an autologous Tetanus Toxoid recall assay (TT assay), e.g. the TT assay as described herein.

In another embodiment, when the ISVD specifically binding to human PD-1 has a sequence identity of more than 90%, such as more than 95% or more than 99%, with one of the VHHs with SEQ ID NOs: 4056, 4094, 4183, or 4210, the ISVD has at least (essentially) the same binding affinity to human PD-1 compared to one of the VHHs with SEQ ID NOs: 4056, 4094, 4183, or 4210, wherein the binding affinity is measured using the same method, such as e.g. SPR.

In another embodiment, when the ISVD specifically binding to human PD-1 has a sequence identity of more than 90%, such as more than 95% or more than 99%, with one of the VHHs with SEQ ID NOs: 4056, 4094, 4183, or 4210, the ISVD has at least (essentially) the same binding affinity to cyno PD-1 compared to one of the VHHs with SEQ ID NOs: 4056, 4094, 4183, or 4210, wherein the binding affinity is measured using the same method, such as e.g. SPR.

In another embodiment, when the ISVD specifically binding to human PD-1 has a sequence identity of more than 90%, such as more than 95% or more than 99%, with one of the VHHs with SEQ ID NOs: 4056, 4094, 4183, or 4210, the ISVD has at least (essentially) the same binding affinity to mouse PD-1 compared to one of the VHHs with SEQ ID NOs: 4056, 4094, 4183, or 4210, wherein the binding affinity is measured using the same method, such as e.g. SPR.

In another embodiment, when the ISVD specifically binding to human PD-1 has a sequence identity of more than 90%, such as more than 95% or more than 99%, with one of the VHHs with SEQ ID NOS: 4056, 4094, 4183, or 4210, the ISVD still specifically binds to human PD-1 and cyno PD-1 with less than 10-fold difference in affinity (KD).

In another embodiment, when the ISVD specifically binding to human PD-1 has a sequence identity of more than 90%, such as more than 95% or more than 99%, with one of the VHHs with SEQ ID NOS:

4056, 4094, 4183, or 4210, the ISVD still specifically binds to human and cyno PD-1 and does not bind to other members of the CD28 receptor family.

In another embodiment, when the ISVD specifically binding to human PD-1 has a sequence identity of more than 90%, such as more than 95% or more than 99%, with one of the VHHs with SEQ ID NOs: 4056, 4094, 4183, or 4210, the ISVD still specifically binds to human and cyno PD-1 and does not bind to CD28, CTLA-4, ICOS, and BTLA.

In another embodiment, when the ISVD specifically binding to human PD-1 has a sequence identity of more than 90%, such as more than 95% or more than 99%, with one of the VHHs with SEQ ID NOs: 4056, 4094, 4183, or 4210, the ISVD blocks the interaction of human PD-1 with human PD-L1 with at least (essentially) the same potency compared to one of the VHHs with SEQ ID NOs: 4056, 4094, 4183, or 4210, wherein the potency is measured in wherein the potency is measured, e.g., in FACS competition assay on CHO cells expressing human PD-1, with hPD-L1 as competitor.

In another embodiment, when the ISVD specifically binding to human PD-1 has a sequence identity of more than 90%, such as more than 95% or more than 99%, with one of the VHHs with SEQ ID NOS: 4056, 4094, 4183, or 4210, the ISVD blocks the interaction of cyno PD-1 with cyno PD-L1 with at least (essentially) the same potency compared to one of the VHHs with SEQ ID NOs: 4056, 4094, 4183, or 4210, wherein the potency is measured, e.g., in FACS competition assay on CHO cells expressing cyno PD-1, with hPD-L1 as competitor.

In another embodiment, when the ISVD specifically binding to human PD-1 has a sequence identity of more than 90%, such as more than 95% or more than 99%, with one of the VHHs with SEQ ID NOS: 4056, 4094, 4183, or 4210, the ISVD blocks the interaction of hPD-1 with (h) PD-L1 with (essentially) the same % inhibition or more compared to one of the VHHs with SEQ ID NOs: 4056, 4094, 4183, or 4210, wherein the % inhibition is determined, e.g., in FACS competition assay on CHO cells expressing human PD-1, with hPD-L1 as competitor.

In another embodiment, when the ISVD specifically binding to human PD-1 has a sequence identity of more than 90%, such as more than 95% or more than 99%, with one of the VHHs with SEQ ID NOs: 4056, 4094, 4183, or 4210, the ISVD has at least (essentially) half of the potency, preferably at least (essentially) the same potency for T-cell activation compared to one of the VHHs with SEQ ID NOs:

4056, 4094, 4183, or 4210, wherein the potency is monitored, e.g., via IFN-gamma production in an autologous Tetanus Toxoid recall assay (TT assay), e.g. the TT assay as described herein.

In another embodiment, when the ISVD specifically binding to human PD-1 has a sequence identity of more than 90%, such as more than 95% or more than 99%, with one of the VHHs with SEQ ID NOS: 4056, 4094, 4183, or 4210, the ISVD has at least 75% of the efficacy, preferably at least 80% or 90% of the efficacy, more preferably at least (essentially) the same efficacy T-cell activation compared to one of the VHHs with SEQ ID NOs: 4056, 4094, 4183, or 4210, wherein the efficacy is monitored, e.g., via IFN-gamma production in an autologous Tetanus Toxoid recall assay (TT assay), e.g. the TT assay as described herein.

In some embodiments, the immunoglobulin single variable domains of the present technology specifically bind to human PD-1 with a dissociation constant (K_{D}) of 5×10⁻⁹ to 10⁻¹² moles/litre or less, and preferably 10⁻⁹ to 10⁻¹² moles/litre or less and more preferably 5×10⁻¹⁰ to 1×10⁻¹¹ moles/litre, such as 1.2×10⁻¹⁰ moles/litre, as determined by Surface Plasmon Resonance, e.g., measured at 25° C.

In some embodiments, the immunoglobulin single variable domains of the present technology specifically bind to human PD-1 with a kₒₙ-rate of between 10⁴ M⁻¹s⁻¹ to about 10⁷ M⁻¹s⁻¹, preferably between 10⁵ M⁻¹s⁻¹ and 10⁷ M⁻¹s⁻¹, more preferably between 10⁵ M⁻¹s⁻¹ and 10⁶ M⁻¹s⁻¹, such as between 1×10⁵ M⁻¹s⁻¹ and 5×10⁵ M⁻¹s⁻¹, such as 3×10⁵ M⁻¹s⁻¹, as determined by Surface Plasmon Resonance, e.g., measured at 25° C.

In some embodiments, the immunoglobulin single variable domains of the present technology specifically bind to human PD-1 with a k_{off} rate between 10⁻³ s⁻¹ (t1/2=0.69 s) and 10⁻⁷ s⁻¹ (providing a near irreversible complex with a t1/2 of multiple days), preferably between 10⁻³ s⁻¹ and 5×10⁻⁶ s⁻¹, more preferably between 5×10⁻⁴ s⁻¹ and 5×10⁻⁶ s⁻¹, such as between 1×10⁻⁴ s⁻¹ and 5×10⁻⁵ s⁻¹, such as 3.5×10⁻⁵ s⁻¹, as determined by Surface Plasmon Resonance, e.g., measured at 25° C.

In some embodiments, the immunoglobulin single variable domains of the present technology specifically bind to cyno PD-1 with a dissociation constant (K_{D}) of 10⁻⁹ to 10⁻¹² moles/litre or less, and preferably 109 to 10⁻¹² moles/litre or less and more preferably 1×10⁻¹⁰ to 1×10⁻¹² moles/litre, such as 7.1x 10⁻¹¹ moles/litre, as determined by Surface Plasmon Resonance, e.g., measured at 25° C.

In some embodiments, the immunoglobulin single variable domains of the present technology specifically bind to cyno PD-1 with a kₒₙ-rate of between 10⁴ M⁻¹s⁻¹ to about 10⁷ M⁻¹s⁻¹, preferably between 5×10⁴ M⁻¹s⁻¹ and 10⁷ M⁻¹s⁻¹, more preferably between 10⁵ M⁻¹s⁻¹ and 10⁷ M⁻¹s⁻¹, such as between 10⁴ M⁻¹s⁻¹ and 5×10⁵ M⁻¹s⁻¹, such as 3.4×10⁵ M⁻¹s⁻¹, as determined by Surface Plasmon Resonance, e.g., measured at 25° C.

In some embodiments, the immunoglobulin single variable domains of the present technology specifically bind to cyno PD-1 with a k_{off} rate between 10⁻³ s⁻¹ (t1/2=0.69 s) and 10⁻⁸ s⁻¹ (providing a near irreversible complex with a t1/2 of multiple days), preferably between 10⁻³ s⁻¹ and 5×10⁻⁷ s⁻¹, more preferably between 10⁻⁴ s⁻¹ and 10⁻⁷ s⁻¹, such as between 1×10⁻⁴ s⁻¹ and 10⁻⁶ s⁻¹, such as 2.4×10⁻⁵ s⁻¹, as determined by Surface Plasmon Resonance, e.g., measured at 25° C.

In some embodiments, the immunoglobulin single variable domains of the present technology specifically bind to human PD-1 and cyno PD-1 with less than 10-fold difference in affinity (K_{D}).

In some embodiments, the immunoglobulin single variable domains of the present technology specifically bind to human and cyno PD-1 and do not bind to other members of the CD28 receptor family.

In some embodiments, the immunoglobulin single variable domains of the present technology specifically bind to human and cyno PD-1 and do not bind to CD28, CTLA-4, ICOS, and BTLA.

In the present technology, the binding of the ISVDs and polypeptides comprising the ISVD to PD-1 can also be measured in assays in which PD-1 is exposed on a cell surface (such as e.g. CHO Flp-In human PD-1 cells, CHO Flp-In cyno PD-1 cells, CHO Flp-In mouse PD-1 cells). The Invitrogen™ Flp-In™ System allows integration and expression of the gene of interest (e.g. encoding human, cyno or mouse PD-1) in mammalian cells (e.g. CHO cells) at a specific genomic location. The Flp-In System involves introduction of a Flp Recombination Target (FRT) site into the genome of the mammalian cell line of choice. An expression vector containing the gene of interest is then integrated into the genome via Flp recombinase-mediated DNA recombination at the FRT site (O'Gorman et al., 1991, RecombinaseMediated Gene Activation and Site-Specific Integration in Mammalian Cells, Science 251, 1351-1355). A preferred assay for measuring binding of the ISVD (or polypeptide comprising the ISVD) to PD-1 exposed on a cell surface is a FACS assay, such as e.g. the FACS assay as described in the examples, wherein binding of the ISVDs or polypeptides to PD-1 expressed on CHO is determined.

In some embodiments, the immunoglobulin single variable domains of the present technology specifically bind PD-1 and block the interaction of PD-1 with PD-L1. In some embodiments, the immunoglobulin single variable domains of the present technology specifically bind hPD-1 and block the interaction of hPD-1 with hPD-L1. In some embodiments, the immunoglobulin single variable domains of the present technology specifically bind cyno PD-1 and block the interaction of cyno PD-1 with cyno PD-L1.

In some embodiments, the immunoglobulin single variable domains of the present technology show a highly potency and/or efficacy (as a single variable domain) for binding PD-1 in competition with PD-L1. In some embodiments, the immunoglobulin single variable domains of the present technology can functionally block PD-1. More specifically, the ISVD can block the interaction between PD-1 and PD-L1. In some embodiments, the immunoglobulin single variable domains of the present technology antagonize an activity of PD-1.

In some embodiments, the immunoglobulin single variable domains of the present technology specifically bind to human PD-1 and block the interaction of human PD-1 with human PD-L1. For example, in FACS competition assay on CHO cells expressing human PD-1, with hPD-L1 as competitor, the immunoglobulin single variable domains of the present technology may have IC50 values of 10⁻⁸ M or lower, more preferably of 5×10⁻⁹ M or lower, or even of 10⁻⁹ M or lower. For example, in such FACS competition assay, the immunoglobulin single variable domains of the present technology may have IC50 values between 10⁻¹¹ M and 10⁻⁸ M, such as between 10⁻¹⁰ M and 10⁻⁹ M, between 10⁻¹⁰ M and 10⁻⁸ M or between 10⁻⁹ M and 10⁻⁸ M, such as 3.08×10⁻⁹ M.

In some embodiments, the immunoglobulin single variable domains of the present technology specifically bind to cyno PD-1 and block the interaction of cyno PD-1 with cyno PD-L1 or human PD-L1. For example, in a FACS competition assay on CHO cells expressing cyno PD-1, with human PD-L1 as competitor, the immunoglobulin single variable domains of the present technology may have IC50 values of 10⁻⁸ M or lower, more preferably of 5×10⁻⁹ M or lower, or even of 10⁻⁹ M or lower. For example, in such FACS competition assay, the immunoglobulin single variable domains of the present technology may have IC50 values between 10⁻¹¹ M and 10⁻⁸ M, such as between 10⁻¹⁰ M and 10⁻⁹ M, between 10⁻¹⁰ M and 10⁻⁸ M or between 10⁻⁹ M and 10⁻⁸ M, such as 3.57×10⁻⁹ M.

In some embodiments, the immunoglobulin single variable domains of the present technology block the binding of human PD-1 to human PD-L1 by at least 50%, such as at least 60%, 70%, 80%, 90%, 95% or even more, as determined by ligand competition, AlphaScreen, or competitive binding assays (such as competition ELISA or competition FACS).

In some embodiments, the immunoglobulin single variable domains of the present technology block the binding of cyno PD-1 to cyno PD-L1 by at least 50%, such as at least 60%, 70%, 80%, 90%, 95% or even more, as determined by ligand competition, AlphaScreen, or competitive binding assays (such as competition ELISA or competition FACS).

In some embodiments, the immunoglobulin single variable domains of the present technology show a highly potency for T cell activation as a single variable domain. T cell activation refers to a process in which mature T cells can express antigen-specific T cell receptors on their surface to recognize their cognate antigens and respond by entering the cell cycle, secreting cytokines or lytic enzymes, and initiating the cell-based functions of the immune system.

T-cell activation as used herein refers to one or more cellular response(s) of a T cell, e.g., a cytotoxic T cell, such as selected from: proliferation, differentiation, cytokine secretion, cytotoxic effector molecule release, cytotoxic activity, expression of activation markers, and redirected target cell lysis.

The polypeptides of the invention are capable of inducing one or more of such responses. Suitable assays to measure T cell activation are known in the art, for instance as described in WO 99/54440 or by Schlereth et al. 2005 (Cancer Immunol. Immunother. 20:1-12), or as exemplified in the examples or below. For example, T cell activation can be measured by assessing (1) proliferation, (2) upregulation of activation markers (e.g., IL2RA/CD25) and/or (3) production of effector cytokines (IFNgamma, TNF-α).

In some embodiments, T cell activation by the ISVDs and polypeptides comprising the same can be monitored by upregulation of CD69, CD25 and various cell adhesion molecules, de novo expression and/or release of cytokines (e.g., IFN-γ, TNF-α, IL-6, IL-2, IL-4 and IL-10), upregulation of granzyme and perforin expression, and/or cell proliferation, membrane blebbing, activation of procaspases 3 and/or 7, fragmentation of nuclear DNA and/or cleavage of caspase substrate poly (ADPribose) polymerase.

In some embodiments, T cell activation is monitored by measurement of IFN-gamma release.

In some embodiments, T cell activation is measured by determining the expression of a number of molecules, namely CD25, CD69, CD71, and HLA-DR, also termed “activation antigens”, by activated proliferating lymphocytes on their surface.′ Such activation antigens are easily evidenced by immunofluorescence assay and flow cytometric analysis using monoclonal antibodies (mAbs) as specific reagents. By two- and three colour immunofluorescent staining it is also possible to identify, within bulk lymphocyte cultures, the T-cell subpopulations involved in the activation process.

In some embodiments, T cell activation can be measured by DNA synthesis assays. DNA synthesis assays use radioactively labelled 3H-thymine to detect DNA within cells. Newly proliferated cells incorporate the label, providing extremely sensitive and accurate detection of cell lines (see e.g., Madhavan 2007, J. Stem Cells Regen. Med. 3:12).

In some embodiments, T cell activation can be measured in a tetanus Toxoid recall assay (TT assay). The assay makes use of Lymphocytes that have previously been exposed to an antigen and that will show an enhanced response upon subsequent re-exposure. Primary lymphocytes from individuals vaccinated against tetanus are used to initiate an in vitro recall antigenic response. Stimulation of such lymphocytes with tetanus toxoid provides a useful assay method to assess the effects of candidate therapeutic molecules, such as checkpoint inhibitors, and as a model for analogous responses to tumor antigens. Lymphocytes are antigenically stimulated following culture with loaded antigen presenting cells (APCs).

Antigen stimulated lymphocytes are cultured in the presence of checkpoint inhibitors to modulate activation response. Endpoints that are assessed in this assay include lymphocyte proliferation, activation markers, and cytokine production (Ticha et al. 2021, A cell-based in vitro assay for testing of immunological integrity of Tetanus toxoid vaccine antigen, Npj Vaccines 6:1-11). In some embodiments, IFN-gamma production is measured following activation of the T lymphocytes.

In some embodiments, the immunoglobulin single variable domains of the present technology have a potency (EC50 values) in a TT assay (e.g. as described in the Examples), monitored via IFN-gamma production, of 10⁷ M or lower, more preferably of 5×10⁻⁸ M or lower, or even of 10⁻⁸ M or lower. For example, in such TT assay, the immunoglobulin single variable domains of the present technology may have a potency (EC50 values) between 10⁻¹¹ M and 10⁻⁷ M, such as between 10⁻¹⁰ M and 10⁻⁷ M, between 10⁻¹⁰ M and 10⁻⁸ M or between 10⁻⁹ M and 10⁷ M, such as 3.6×10⁻⁸ M, 5.68×10⁻⁸ M or 5.8×10 M.

In some embodiments, a polypeptide comprising at least two immunoglobulin single variable domains of the present technology has a potency (EC50 values) in a TT assay (e.g. as described in the Examples), monitored via IFN-gamma production, of 10⁻⁸ M or lower, more preferably of 5×10⁻⁹ M or lower, or even of 10⁻⁹ M or lower. For example, in such TT assay, the immunoglobulin single variable domains of the present technology may have a potency (EC50 values) between 10⁻¹¹ M and 10⁻⁸ M, such as between 10⁻¹⁰ M and 10⁻⁸ M, between 10⁻¹⁰ M and 10⁻⁹ M or between 10⁻⁹ M and 10⁻⁸ M, such as 8.4×10 9 M, or 3×10⁻⁹ M.

In some embodiments, the immunoglobulin single variable domains of the present technology have an efficacy in a TT assay (e.g., as described in the Examples), monitored via IFN-gamma production, of at least 50%, such as at least 60%, 70%, 80%, 90%, 95%.

The T Cell Activation Bioassay (NFAT) is a bioluminescent cell-based assay that consists of a genetically engineered Jurkat T cell line that expresses a luciferase reporter driven by an NFAT-response element (NFAT-RE). When the TCR/CD3 Effector Cells (NFAT) are engaged, the TCR transduces intracellular signals resulting in NFAT-RE-mediated luminescence. The bioluminescent signal is detected and quantified using Bio-Glo™ Luciferase Assay System and a standard luminometer, such as the GloMax® Discover System.

In some embodiments, the immunoglobulin single variable domains of the present technology have a potency (IC50 values) in a NFAT assay (e.g., as described in the Examples) of 10⁻⁷ M or lower, of 5×10⁻⁸ M or lower, more preferably of 10⁻⁸ M or lower, or even of 5×10⁻⁹ M or lower, or even 10⁻⁹ or lower.

For example, in such NFAT assay, the immunoglobulin single variable domains of the present technology may have a potency (IC50 values) between 10⁻¹⁰ M and 10⁻⁷ M, such as between 10⁻¹⁰ M and 10⁻⁸ M or between 10⁻¹⁰ M and 10⁻⁹ M, such as 0.5×10⁻⁹ M or 7.75×10⁻⁹ M.

In some embodiments, the immunoglobulin single variable domains of the present technology block the binding of human PD-L1 to human PD-1 by at least 50%, such as at least 60%, 70%, 80%, 90%, 95% or even more, as determined in a NFAT assay.

In some embodiments, the immunoglobulin single variable domains of the present technology block the binding of human or cyno PD-L1 to cyno PD-1 by at least 50%, such as at least 60%, 70%, 80%, 90%, 95% or even more, as determined in a NFAT assay.

In another embodiment, when the ISVD specifically binding to human PD-1 has a sequence identity of more than 90%, such as more than 95% or more than 99%, with one of the VHHs with SEQ ID NOS: 4056, 4094, 4183, or 4210, the ISVD blocks the interaction of hPD-1 with (h) PD-L1 with (essentially) the same % inhibition or more compared to one of the VHHs with SEQ ID NOs: 4056, 4094, 4183, or 4210, wherein the % inhibition is determined in a NFAT assay.

### 5.3 Polypeptides and Constructs

The process of designing/selecting and/or preparing a polypeptide, starting from an immunoglobulin single variable domain such as a VHH, humanized VHH, camelized VH, domain antibody or dAb, is also referred to herein as “formatting” said immunoglobulin single variable domain; and an immunoglobulin single variable domain that is made part of a polypeptide is said to be “formatted” or to be “in the format of” said polypeptide. Examples of ways in which an immunoglobulin single variable domain can be formatted, and examples of such formats will be clear to the skilled person based on the disclosure herein; and such formatted immunoglobulin single variable domain form a further aspect of the present technology.

For example, and without limitation, one or more immunoglobulin single variable domains may be used as a “binding unit”, “binding domain” or “building block” (these terms are used interchangeable) for the preparation of a polypeptide, which may optionally contain one or more further immunoglobulin single variable domains that can serve as a binding unit (i.e., against the same or another epitope on PD-1 and/or against one or more other antigens, proteins or targets than PD-1).

The present technology also provides a polypeptide or construct that comprises or essentially consists of one or more immunoglobulin single variable domain. In one embodiment, the polypeptides further comprises one or more other groups, residues, moieties or binding units, optionally linked via one or more linkers. The one or more other groups, residues, moieties or binding units can be any groups, residues, moieties or binding units known in the art. In one embodiment the one or more other groups, residues, moieties or binding units are amino acid sequences, so that the resulting polypeptide is a fusion (protein) or fusion (polypeptide).

The further groups, residues, moieties, binding units or amino acid sequences may or may not provide further functionality to the ISVD, polypeptide or construct and may or may not modify the properties of the ISVD, polypeptide and/or construct. Such groups, residues, moieties or binding units may for example be chemical groups, residues, moieties, which may or may not by themselves be biologically and/or pharmacologically active. For example, and without limitation, such groups may be linked to the one or more ISVD and/or polypeptide so as to provide a “derivative” of the ISVD and/or polypeptide.

The one or more further amino acid sequence may be any suitable and/or desired amino acid sequences. The further amino acid sequences may or may not change, alter or otherwise influence the (biological) properties of the ISVD or polypeptide, and may or may not add further functionality to the ISVD or the polypeptide. Preferably, the further amino acid sequence is such that it confers one or more desired properties or functionalities to the ISVD or the polypeptide. Example of such amino acid sequences will be clear to the skilled person and may generally comprise all amino acid sequences that are used in peptide fusions based on conventional antibodies and fragments thereof (including but not limited to ScFv's and single domain antibodies). Reference is for example made to the review by Holliger and Hudson, Nature Biotechnology, 23, 9, 1126-1136 (2005). For example, such an amino acid sequence may be an amino acid sequence that increases the half-life, the solubility, or the absorption, reduces the immunogenicity or the toxicity, eliminates or attenuates undesirable side effects, and/or confers other advantageous properties to and/or reduces the undesired properties of the ISVD or polypeptide, compared to the ISVD or polypeptide per se. Some non-limiting examples of such amino acid sequences are serum proteins, such as human serum albumin (see for example WO 00/27435) or haptenic molecules (for example haptens that are recognized by circulating antibodies, see for example WO 98/22141).

The further amino acid sequence may also provide a second binding site, which binding site may be directed against any desired protein, polypeptide, antigen, antigenic determinant or epitope (including but not limited to the same protein, polypeptide, antigen, antigenic determinant or epitope against which the ISVD is directed, or a different protein, polypeptide, antigen, antigenic determinant or epitope).

In one embodiment, the one or more other groups, residues, moieties or binding units are binding units. In one embodiment the binding unit is an immunoglobulin sequence. In one embodiment the binding unit is an immunoglobulin single variable domain, such as a VHH, humanized VHH, camelized VH, domain antibody, single domain antibody or dAb.

In one embodiment, the one or more other groups, residues, or moieties comprise the introduction of one or more detectable labels or other signal-generating groups or moieties, depending on the intended use of the labelled ISVD. Suitable labels and techniques for attaching, using and detecting them will be clear to the skilled person, and for example include, but are not limited to, fluorescent labels (such as fluorescein, isothiocyanate, rhodamine, phycoerythrin, phycocyanin, allophycocyanin, o-phthaldehyde, and fluorescamine and fluorescent metals such as ¹⁵²Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes (such as ³H, ¹²⁵I, ³²P, ³⁵S, ¹⁴C, ⁵¹Cr, ³⁶Cl, ⁵⁷Co, ⁵⁸Co, ⁵⁹Fe, and ⁷⁵Se), metals, metal chelates or metallic cations (for example metallic cations such as ⁹⁹mTc, ¹²³I, ¹¹¹In, ¹³¹I, ⁹⁷Ru, ⁶⁷Cu, ⁶⁷Ga, ⁶⁸Ga and ⁸⁹Zr or other metals or metallic cations that are particularly suited for use in in vivo, in vitro or in situ diagnosis and imaging, such as (¹⁵⁷Gd, ⁵⁵Mn, ¹⁶²Dy, ⁵²Cr, and ⁵⁶Fe), as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person, and for example include moieties that can be detected using NMR or ESR spectroscopy.

Such labelled ISVDs, polypeptides and constructs may for example be used for in vitro, in vivo or in situ assays (including immunoassays known per se such as ELISA, RIA, EIA and other “sandwich assays”, etc.) as well as for in vivo diagnostic, imaging and/or therapeutic purposes, depending on the choice of the specific label.

Examples of imaging moieties are provided in Agdeppa and Spilker 2009 (A review of imaging agent development. AAPS J. 11 (2): 286-99). For instance, the imaging moiety present in the ISVD, polypeptide or construct of the present technology may be suitable for radiotherapy and for radio/fluorescence-guided cancer surgery. For instance, the imaging moiety may comprise radioactive isotopes that can be used for diagnostic and therapeutic proposes. For instance, the imaging moiety may be a contrast agent. For instance, the imaging moiety may be a non-radioactive medical isotope. For instance, the imaging moiety may include desferrioxamine (DFO), such as used for ⁸⁹Zirconium-DFO-labeling. For instance, the imaging moiety may be a fluorophore such as Alexa 647 or pHAb.

Examples of antibody-radionuclide conjugates are provided in Steiner and Neri 2011 (Antibody-Radionuclide Conjugates for Cancer Therapy: Historical Considerations and New Trends. Clin. Cancer Res. 17 (20): 6406-6416). For instance, the ISVDs, polypeptides and constructs labelled with therapeutic radionuclides, can be used in the development of anticancer strategies, with the aim of concentrating radioactivity at the tumor site and sparing normal tissues. As such, the present technology also provides ISVDs, polypeptides and constructs of the present technology that are radioactively labelled for us in (radio) therapy, such as cancer (radio) therapy.

Monovalent polypeptides comprise or essentially consist of only one binding unit (such as e.g., immunoglobulin single variable domains). Polypeptides that comprise two or more binding units (such as e.g., immunoglobulin single variable domains) will also be referred to herein as “multivalent” polypeptides, and the binding units/immunoglobulin single variable domains present in such polypeptides will also be referred to herein as being in a “multivalent format”. For example a “bivalent” polypeptide may comprise two immunoglobulin single variable domains, optionally linked via a linker sequence, whereas a “trivalent” polypeptide may comprise three immunoglobulin single variable domains, optionally linked via two linker sequences; whereas a “tetravalent” polypeptide may comprise four immunoglobulin single variable domains, optionally linked via three linker sequences; whereas a “pentavalent” polypeptide may comprise five immunoglobulin single variable domains, optionally linked via four linker sequences; whereas a “hexavalent” polypeptide may comprise six immunoglobulin single variable domains, optionally linked via five linker sequences, etc. Examples of monovalent polypeptides are depicted in Table A-2 (SEQ ID NOs: 1 to 448, such as SEQ ID NO: 402, 331, 332, 375, 212, 139, 238), Table A-3 (SEQ ID NOs: 714-1871, such as SEQ ID NO: 793, 1505, 1092, 1085, 1078, 1083, 1486), Table A-4 (SEQ ID Nos: 2857 to 3662, such as SEQ ID NOs: 3395, 3376, 3447, 3446, 3643, 3653, 2859), and Table A-5 (SEQ ID NOs: 4049-4378, such as SEQ ID NO: 4056, 4094, 4183, 4210). Examples of bivalent polypeptides and trivalent polypeptides are depicted in Table A-13 (SEQ ID NOs: 4631 to 4644).

In a multivalent polypeptide, the two or more immunoglobulin single variable domains may be the same or different, and may be directed against the same antigen or antigenic determinant (for example against the same part(s) or epitope(s) or against different parts or epitopes) or may alternatively be directed against different antigens or antigenic determinants; or any suitable combination thereof. Polypeptides that contain at least two binding units (such as e.g., immunoglobulin single variable domains) in which at least one binding unit is directed against a first antigen (i.e., PD-1) and at least one binding unit is directed against a second antigen (i.e., different from PD-1) will also be referred to as “multispecific” polypeptides, and the binding units (such as e.g., immunoglobulin single variable domains) present in such polypeptides will also be referred to herein as being in a “multispecific format”. Thus, for example, a “bispecific” polypeptide is a polypeptide that comprises at least one immunoglobulin single variable domain directed against a first antigen (i.e., PD-1) and at least one further immunoglobulin single variable domain directed against a second antigen (i.e., different from PD-1), whereas a “trispecific” polypeptide is a polypeptide that comprises at least one immunoglobulin single variable domain directed against a first antigen (i.e., PD-1), at least one further immunoglobulin single variable domain directed against a second antigen (i.e., different from PD-1) and at least one further immunoglobulin single variable domain directed against a third antigen (i.e., different from both PD-1 and the second antigen); etc

In one embodiment, the polypeptide may further comprise one or more other groups, residues, moieties or binding units, optionally linked via one or more peptidic linkers, in which said one or more other groups, residues, moieties or binding units provide the polypeptide with increased (in vivo) half-life, compared to the corresponding polypeptide without said one or more other groups, residues, moieties or binding units. In vivo half-life extension means, for example, that the polypeptide has an increased half-life in a mammal, such as a human subject, after administration. Half-life can be expressed for example as t1/2beta.

The type of groups, residues, moieties or binding units is not generally restricted and may for example be chosen from the group consisting of a polyethylene glycol molecule, serum proteins or fragments thereof, binding units that can bind to serum proteins, an Fc portion, and small proteins or peptides that can bind to serum proteins.

More specifically, said one or more other groups, residues, moieties or binding units that provide the polypeptide with increased half-life can be chosen from the group consisting of binding units that can bind to serum albumin, such as human serum albumin, or a serum immunoglobulin, such as IgG. In one embodiment, said one or more other groups, residues, moieties or binding units that provide the polypeptide with increased half-life is a binding unit that can bind to human serum albumin. In one embodiment, the binding unit is an ISVD.

For example, WO 2004/041865 and WO 2006/122787 describes ISVDs binding to serum albumin (and in particular against human serum albumin) that can be linked to other proteins (such as one or more other ISVDs binding to a desired target) in order to increase the half-life of said protein. These ISVDs include the ISVDs called Alb-1 (SEQ ID NO: 52 in WO 2006/122787) and humanized variants thereof, such as Alb-8 (SEQ ID NO: 62 in WO 2006/122787). Again, these can be used to extend the half-life of therapeutic proteins and polypeptide and other therapeutic entities or moieties. Moreover, WO 2012/175400 describes a further improved version of Alb-1, called Alb-23.

In one embodiment, the polypeptide comprises a serum albumin binding moiety selected from Alb-1, Alb-3, Alb-4, Alb-5, Alb-6, Alb-7, Alb-8, Alb-9, Alb-10 (WO 2006/122787) and Alb-23. In one embodiment, the serum albumin binding moiety is Alb-8 or Alb-23 or its variants, as shown on pages 7-9 of WO 2012/175400. In one embodiment, the serum albumin binding moiety is selected from the albumin binders described in WO 2012/175741, WO2015/173325, WO2017/080850, WO2017/085172, WO2018/104444, WO2018/134235, and WO2018/134234. Some serum albumin binders are also shown in Table A-11. In one embodiment, the serum albumin binder is AlbX00001 (SEQ ID NO: 2415). In one preferred embodiment the serum albumin binder is Alb23002 (SEQ ID NO: 2416). Examples of polypeptides that comprise ALB23002 are depicted in Table A-13 (SEQ ID NOS: 4631-4633, 4635, 4637, 4639, 4641, 4643, 4644).

In one embodiment, a further component of the polypeptide of the present technology is an ISVD that specially binds to human serum albumin and comprises
i. a CDR1 (Abm numbering) that is the amino acid sequence of SEQ ID NO: 2436; or an amino acid sequence with 2 or 1 amino acid difference with SEQ ID NO: 2436;
ii. a CDR2 (Abm numbering) that is the amino acid sequence of SEQ ID NO: 2438 or an amino acid sequence with 2 or 1 amino acid difference with SEQ ID NO: 2438; and
iii. a CDR3 (Abm numbering) that is the amino acid sequence of SEQ ID NO: 2440 or an amino acid sequence with 2 or 1 amino acid difference with SEQ ID NO: 2440.

In one embodiment, the ISVD that specifically binds to human serum albumin comprises a CDR1 that is the amino acid sequence of SEQ ID NO: 2436, a CDR2 that is the amino acid sequence of SEQ ID NO: 2438 and a CDR3 that is the amino acid sequence of SEQ ID NO: 2440.

Examples of such an ISVD that specifically binds to human serum albumin have one or more, or all, framework regions as indicated for ISVD ALB23002 in Table A-7 (in addition to the CDRs as defined above). In one embodiment, it is an ISVD comprising or consisting of the full amino acid sequence of ISVD ALB23002 (SEQ ID NO: 2416, see Table A-7 and A-11).

Also in another embodiment, the amino acid sequence of an ISVD binding to human serum albumin may have a sequence identity of more than 90%, such as more than 95% or more than 99%, with SEQ ID NO: 2416, wherein the CDRs are as defined above. In one embodiment, the ISVD binding to human serum albumin comprises or consists of the amino acid sequence of SEQ ID NO: 2416.

When such an ISVD binding to human serum albumin has 2 or 1 amino acid difference in at least one CDR relative to a corresponding reference CDR sequence (as defined above), the ISVD has at least half of the binding affinity, or at least the same binding affinity, to human serum albumin compared to construct ALB23002 (SEQ ID NO: 2416), wherein the binding affinity is measured using the same method, such as SPR.

In one embodiment, when such an ISVD binding to human serum albumin has a C-terminal position in the polypeptide or construct, it exhibits a C-terminal extension, such as a C-terminal alanine (A) or glycine (G) extension. In one embodiment such an ISVD is selected from SEQ ID NOs: 2403, 2404, 2406, 2408, 2409, 2410, 2411, 2412, 2413, and 2414 (see Table A-11). In another embodiment, the ISVD binding to human serum albumin has another position than the C-terminal position in the polypeptide (i.e. is not the C-terminal ISVD of the polypeptide of the technology). In one embodiment such an ISVD is selected from SEQ ID NOs: 2401, 2402, 2405, 2407, 2415 and 2416 (see Table A-11).

In one embodiment, the ISVD or polypeptide of the present technology may be linked (optionally via a suitable linker or hinge region) to one or more constant domains (for example, 2 or 3 constant domains that can be used as part of/to form an Fc portion), to an Fc portion and/or to one or more antibody parts, fragments or domains that confer one or more effector functions to the ISVD or polypeptide and/or may confer the ability to bind to one or more Fc receptors. For example, for this purpose, and without being limited thereto, the one or more further amino acid sequences may comprise one or more CH2 and/or CH3 domains of an antibody, such as from a heavy chain antibody (as described herein) and more preferably from a conventional human 4-chain antibody; and/or may form (part of) and Fc region, for example from IgG (e.g. from IgG1, IgG2, IgG3 or IgG4), from IgE or from another human Ig such as IgA, IgD or IgM. For example, WO 94/04678 describes heavy chain antibodies comprising a Camelid VHH domain or a humanized derivative thereof in which the Camelidae CH2 and/or CH3 domain have been replaced by human CH2 and CH3 domains, so as to provide an immunoglobulin that consists of 2 heavy chains each comprising a VHH and human CH2 and CH3 domains (but no CH1 domain), which immunoglobulin has the effector function provided by the CH2 and CH3 domains and which immunoglobulin can function without the presence of any light chains. Other amino acid sequences that can be suitably linked to the ISVD or polypeptide of the present technology so as to provide an effector function will be clear to the skilled person and may be chosen on the basis of the desired effector function(s). Reference is for example made to WO 04/058820, WO 99/42077, WO 02/056910 and WO 05/017148, as well as the review by Holliger and Hudson, supra; and to WO 09/068628. Coupling of ISVD or polypeptide to an Fc portion may also lead to an increased half-life, compared to the corresponding ISVD or polypeptide. For some applications, the use of an Fc portion and/or of constant domains (i.e. CH2 and/or CH3 domains) that confer increased half-life without any biologically significant effector function may also be suitable or even preferred. Other suitable constructs comprising one or more ISVDs or polypeptides and one or more constant domains with increased half-life in vivo will be clear to the skilled person and may for example comprise ISVDs or polypeptides linked to a CH3 domain, optionally via a linker sequence.

Generally, any fusion protein or derivatives with increased half-life will preferably have a molecular weight of more than 50 kD, the cut-off value for renal absorption.

The components, e.g. the ISVDs, of the polypeptide may be linked to each other by one or more suitable linkers, such as peptidic linkers. The use of linkers to connect two or more (poly) peptides is well known in the art. Exemplary peptidic linkers are shown in Table A-12. One often used class of peptidic linker are known as the “Gly-Ser” or “GS” linkers. These are linkers that essentially consist of glycine (G) and serine(S) residues, and usually comprise one or more repeats of a peptide motif such as the GGGGS (SEQ ID NO: 2419) motif (for example, have the formula (Gly-Gly-Gly-Gly-Ser)n (SEQ ID NO: 4795) in which n may be 1, 2, 3, 4, 5, 6, 7 or more). Some often-used examples of such GS linkers are 9GS linkers (GGGGSGGGS, SEQ ID NO: 2422), 15GS linkers (n=3; SEQ ID NO: 2424) and 35GS linkers (n=7; SEQ ID NO: 2429). Reference is for example made to Chen et al. 2013 (Adv. Drug Deliv. Rev. 65 (10): 1357-1369) and Klein et al. 2014 (Protein Eng. Des. Sel. 27 (10): 325-330). In one embodiment, 9GS linkers to link the components of the polypeptide to each other, are used. Examples of ISVD formats with a 9GS linker are depicted in Table A-13 (SEQ ID NOs: 4631-4644). In one embodiment, 35GS linkers to link the components of the polypeptide to each other, are used.

The present technology also provides sequence optimized ISVDs and polypeptides that show increased stability upon storage during stability studies. In one embodiment, the sequence optimized ISVDs and polypeptides show reduced pyroglutamate post-translational modification of the N-terminus and hence have increased product stability. Pyroglutamaat modification leads to heterogeneity of the final product and needs to be avoided. The possibility of pGlu post-translational modification of the N-terminus was eliminated by changing the N-terminal Glutamic acid (E) into an Aspartic acid (D) which lead to increased product stability. Accordingly, the present invention also relates to ISVDs and polypeptides as described above wherein the Glutamic acid at position 1 (said position determined according to Kabat numbering) is changed into an Aspartic acid (E1D). Examples of ISVDs and polypeptides with aspartic acid at position 1 (according to Kabat numbering) are depicted in Table A-2 (SEQ ID NOs: 1, 239, 328-330), Table A-3 (SEQ ID NOs: 714, 930, 1137, Table A-4 (SEQ ID NOs: 2857-2859, 3594-3595, 3637, 3640-3642), and Table A-5 (SEQ ID NOs: 4049, 4270-4272, 4378).

The present technology also provides sequence optimized ISVDs and polypeptides that are “humanized”, i.e. in which one or more amino acid residues in the amino acid sequence of said naturally occurring VHH sequence (and in particular in the framework sequences) are replaced by one or more of the amino acid residues that occur at the corresponding position(s) in a VH domain from a conventional 4-chain antibody from a human being (e.g. indicated above). Accordingly, the present invention also relates to ISVDs and polypeptides as described above that are humanized. Examples of such humanized ISVDs are depicted in Table A-2 (SEQ ID NOs: 1-401, 403-488), Table A-3 (SEQ ID NOS: 714-792, 794-1871Table A-4 (SEQ ID NOs: 2857-3394, 3396-3662), Table A-5 (SEQ ID NOs: 4094-4055, or 4057-4378).

The present technology also provides sequence optimized ISVDs and polypeptides that exhibit reduced binding by pre-existing antibodies present in human serum. To this end, in one embodiment, the polypeptide comprises a valine (V) at amino acid position 11 and a leucine (L) at amino acid position 89 (according to Kabat numbering) in at least one ISVD. In one embodiment, the polypeptide comprises a valine (V) at amino acid position 11 and a leucine (L) at amino acid position 89 (according to Kabat numbering) in each ISVD. Accordingly, the present invention also relates to ISVDs and polypeptides as described above that have been sequence optimized with a valine (V) at amino acid position 11 and a leucine (L) at amino acid position 89 (according to Kabat numbering) in at least one ISVD, such as in all ISVDs. Examples of ISVDs and polypeptides that comprises a valine (V) at amino acid position 11 and a leucine (L) at amino acid position 89 (according to Kabat numbering) in at least one ISVD are depicted in Table A-2 (SEQ ID NOs: 1-401, 403-488), Table A-3 (SEQ ID NOs: 714-792, 794-1871, such as 1505, 1092, 1085, 1078, 1083, 1486), Table A-4 (SEQ ID NOs: 2857-3394, 3396-3662), Table A-5 (SEQ ID NOs: 4094-4055, 4057-4378, such as 4094, 4183, 4210).

In one embodiment, the ISVD or polypeptide has a C-terminal end of the sequence VTVSS(X)n (SEQ ID NO: 4613), in which n is 1 to 10, preferably 1 to 5, such as 1, 2, 3, 4 or 5, and in which each X is an amino acid residue that is independently chosen. In one embodiment, the polypeptide comprises such an ISVD at its C-terminal end. In one embodiment, n is 1 or 2, such as 1. In one embodiment, X is a naturally occurring amino acid. In one embodiment, X is chosen from the group consisting of alanine (A), glycine (G), valine (V), leucine (L) or isoleucine (I).

In another embodiment the polypeptide comprises a lysine (K) or glutamine (Q) at position 110 (according to Kabat numbering) in at least one ISVD. In another embodiment, the ISVD comprises a lysine (K) or glutamine (Q) at position 112 (according to Kabat numbering) in at least one ISVD. In these embodiments, the C-terminus of the ISVD is VKVSS (SEQ ID NO: 4614), VQVSS (SEQ ID NO: 4615), VTVKS (SEQ ID NO: 4616), VTVQS (SEQ ID NO: 4617), VKVKS (SEQ ID NO: 4618), VKVQS (SEQ ID NO: 4619), VQVKS (SEQ ID NO: 4620), or VQVQS (SEQ ID NO: 4621) such that after addition of a single alanine the C-terminus of the polypeptide for example comprises the sequence VTVSSA (SEQ ID NO: 4622), VKVSSA (SEQ ID NO: 4623), VQVSSA (SEQ ID NO: 4624), VTVKSA (SEQ ID NO: 4625), VTVQSA (SEQ ID NO: 4626), VKVKSA (SEQ ID NO: 4627), VKVQSA (SEQ ID NO: 4628), VQVKSA (SEQ ID NO: 4629), or VQVQSA (SEQ ID NO: 4630). In one embodiment, the polypeptide comprises a valine (V) at amino acid position 11 and a leucine (L) at amino acid position 89 (according to Kabat numbering) in each ISVD, optionally a lysine (K) or glutamine (Q) at position 110 (according to Kabat numbering) in at least one ISVD, and comprises an extension of 1 to 5 (naturally occurring) amino acids (as defined above), such as a single alanine (A) extension, at the C-terminus of the C-terminal ISVD, such that the C-terminus of the polypeptide for example comprises the sequence VTVSSA (SEQ ID NO: 4622), VKVSSA (SEQ ID NO: 4623), or VQVSSA (SEQ ID NO: 4624). See e.g. WO2012/175741 and WO2015/173325 for further information in this regard.

In one embodiment, the polypeptide of the present technology comprises or essentially consists of one or more of the ISVDs selected from the group consisting of the amino acid sequence of SEQ ID NOs: 1 to 488, 714-1871, 2857-3662, 4094-4378. In one embodiment, the polypeptides further comprises one or more other groups, residues, moieties or binding units (as described herein), optionally linked via one or more linkers. In one embodiment, the polypeptide is selected from the group consisting of the amino acid sequences of SEQ ID NOs: 4631 to 4644.

In another embodiment, the polypeptide may have a sequence identity of more than 90%, such as more than 95% or more than 99%, with one of SEQ ID NOs: 4631 to 4644. In another embodiment, the polypeptide may have a sequence identity of more than 90%, such as more than 95% or more than 99%, with one of SEQ ID NOs: 4631 to 4644, wherein the sequences are as defined above. In one embodiment, the polypeptide comprises or consists of any one of the amino acid sequences of SEQ ID NOs: 4631 to 4644.

In some embodiments, the polypeptide or construct of the present technology specifically bind to human PD-1 with a dissociation constant (K_{D}) of 10⁻⁸ to 10⁻¹¹ moles/litre, such as 5×10⁹ to 10⁻¹¹ moles/litre or less, and preferably 5×10⁻⁹ to 10⁻¹¹ or 10⁻⁹ to 5×10⁻¹¹ moles/litre or less and more preferably 5×10⁻¹⁰ to 10⁻¹⁰ moles/litre, as determined by Surface Plasmon Resonance, e.g., measured at 25° C.

In some embodiments, the polypeptide or construct of the present technology specifically bind to human PD-1 with a kon-rate of between 10⁴ M⁻¹ s⁻¹ to about 10⁷ M⁻¹ s⁻¹, such as 10⁵ M⁻¹ s⁻¹ to about 10⁷ M⁻¹ s⁻¹, preferably between 5×10⁵ M⁻¹ s⁻¹ and 10⁷ M⁻¹ s⁻¹, more preferably between 10⁶ M⁻¹ s⁻¹ and 10⁷ M⁻¹ s⁻¹, such as between 10⁶ M⁻¹ s⁻¹ and 5×10⁶ M⁻¹ s⁻¹, as determined by Surface Plasmon Resonance, e.g., measured at 25° C.

In some embodiments, the polypeptide or construct of the present technology specifically bind to human PD-1 with a koff rate between 5×10⁻³ s⁻¹ (t1/2=0.69 s) and 10⁻⁶ s⁻¹ (providing a near irreversible complex with a t1/2 of multiple days), preferably between 10⁻³ s⁻¹ and 10⁻⁶ s⁻¹, more preferably between 5×10⁻⁴ s⁻¹ and 10⁻⁶ s⁻¹, such as between 5×10⁻⁴ s⁻¹ and 10⁻⁵ s⁻¹, as determined by Surface Plasmon Resonance, e.g., measured at 25° C.

In some embodiments, the polypeptide or construct of the present technology specifically bind to cyno PD-1 with a dissociation constant (K_{D}) of 10⁻⁸ to 10⁻¹¹ moles/litre, such as 5×10⁹ to 10⁻¹¹ moles/litre or less, and preferably 5×10⁻⁹ to 10⁻¹¹ or 10⁻⁹ to 5×10⁻¹¹ moles/litre or less and more preferably 5×10⁻¹⁰ to 10⁻¹⁰ moles/litre, as determined by Surface Plasmon Resonance, e.g., measured at 25° C.

In some embodiments, the polypeptide or construct of the present technology specifically bind to cyno PD-1 with a kon-rate of between 10⁴ M⁻¹ s⁻¹ to about 10⁷ M⁻¹ s⁻¹, such as 10⁵ M⁻¹ s⁻¹ to about 10⁷ M⁻¹ s⁻¹, preferably between 5×10⁵ M⁻¹ s⁻¹ and 10⁷ M⁻¹ s⁻¹, more preferably between 10⁶ M⁻¹ s⁻¹ and 10⁷ M⁻¹ s⁻¹, such as between 10⁶ M⁻¹ s⁻¹ and 5×10⁶ M⁻¹ s⁻¹, as determined by Surface Plasmon Resonance, e.g., measured at 25° C.

In some embodiments, the polypeptide or construct of the present technology specifically bind to cyno PD-1 with a koff rate between 5×10⁻³ s⁻¹ (t1/2=0.69 s) and 10⁻⁶ s⁻¹ (providing a near irreversible complex with a t1/2 of multiple days), preferably between 10⁻³ s⁻¹ and 10⁻⁶ s⁻¹, more preferably between 5×10⁻⁴ s⁻¹ and 10⁻⁶ s⁻¹, such as between 5×10⁻⁴ s⁻¹ and 10⁻⁵ s⁻¹, as determined by Surface Plasmon Resonance, e.g., measured at 25° C.

In some embodiments, the polypeptide or construct of the present technology specifically bind to human PD-1 and cyno PD-1 with less than 10-fold difference in affinity (KD).

In some embodiments, the polypeptide or construct of the present technology specifically bind to human and cyno PD-1 and do not bind to CD28, CTLA-4, ICOS, and BTLA or other members of the CD28 receptor family.

In some embodiments, the polypeptide or construct of the present technology can functionally block PD-1. More specifically, the ISVD can block the interaction between PD-1 and PD-L1. In some embodiments, the immunoglobulin single variable domains of the present technology antagonize an activity of PD-1.

In some embodiments, the polypeptide or construct of the present technology specifically bind to human PD-1 and block the interaction of human PD-1 with human PD-L1. For example, in FACS competition assay on CHO cells expressing human PD-1, with hPD-L1 as competitor, the immunoglobulin single variable domains of the present technology may have EC50 values of 10⁻⁸ M or lower, more preferably of 5·10⁹ M or lower, or even of 10⁹ M or lower. For example, in such FACS competition assay, the immunoglobulin single variable domains of the present technology may have EC50 values between 10⁻¹¹ M and 10⁻⁸ M, such as between 10⁻¹⁰ M and 10⁻⁹ M, between 10⁻¹⁰ M and 10⁻⁸ M or between 10⁻⁹ M and 10⁻⁸ M.

In some embodiments, the polypeptide or construct of the present technology specifically bind to cyno PD-1 and block the interaction of cyno PD-1 with cyno PD-L1. For example, in FACS competition assay on CHO cells expressing cyno PD-1, with cyno PD-L1 as competitor, the immunoglobulin single variable domains of the present technology may have EC50 values of 5·10⁻⁸ M or lower, 10⁻⁸ M or lower, more preferably of 5·10⁻⁹ M or lower, or even of 10⁻⁹ M or lower. For example, in such FACS competition assay, the immunoglobulin single variable domains of the present technology may have EC50 values between 10⁻¹¹ M and 10⁻⁸ M, such as between 10⁻¹⁰ M and 10⁻⁹ M, between 10⁻¹⁰ M and 10⁻⁸ M or between 10⁻⁹ M and 10⁻⁸ M.

In some embodiments, the polypeptide or construct of the present technology specifically bind to human PD-1 and block the interaction of human PD-1 with human PD-L1. For example, in NFTA reporter assay on CHO cells expressing human PD-L1, with PD1 Jurkat NFTA effector cells, the immunoglobulin single variable domains of the present technology may have IC50 values of 10⁻⁸ M or lower, more preferably of 5·10⁻⁹ M or lower, or even of 10⁻⁹ M or lower. For example, in such NFTA reporter assay, the immunoglobulin single variable domains of the present technology may have IC50 values between 10⁻¹¹ M and 10⁻⁸ M, such as between 10⁻¹⁰ M and 10⁻⁹ M, between 10⁻¹⁰ M and 10⁻⁸ M or between 10⁻⁹ M and 10⁻⁸ M.

In some embodiments, the polypeptide or construct of the present technology block the binding of human PD-1 to human PD-L1 by at least 20%, such as at least 30%, 40%, 50%, 60%, 70%, 80%, 90%, 95% or even more, as determined by ligand competition, AlphaScreen, or competitive binding assays (such as competition ELISA or competition FACS).

In some embodiments, the polypeptide or construct of the present technology block the binding of cyno PD-1 to cyno PD-L1 by at least 20%, such as at least 30%, 40%, 50%, 60%, 70%, 80%, 90%, 95% or even more, as determined by ligand competition, AlphaScreen, or competitive binding assays (such as competition ELISA or competition FACS).

In some embodiments, the polypeptide or construct of the present technology have a potency (EC50 values) in a Tetanus Toxoid recall assay (e.g. as described in the Examples) of 10⁷ M or lower, more preferably of 5×10⁻⁸ M or lower, or even of 10⁻⁸ M or lower.

In some embodiments, the polypeptide or construct of the present technology have an efficacy in a Tetanus Toxoid recall assay (e.g. as described in the Examples), monitored via IFN-gamma production, of at least 50%, such as at least 60%, 70%, 80%, 90%, 95%.

### 5.4 Nucleic Acid Molecules

Also provided is a nucleic acid molecule encoding the ISVD and/or polypeptide of the present technology.

A “nucleic acid molecule” (used interchangeably with “nucleic acid”) is a chain of nucleotide monomers linked to each other via a phosphate backbone to form a nucleotide sequence. A nucleic acid may be used to transform/transfect a host cell or host organism, e.g. for expression and/or production of a polypeptide. Suitable hosts or host cells for production purposes will be clear to the skilled person, and may for example be any suitable fungal, prokaryotic or eukaryotic cell or cell line or any suitable fungal, prokaryotic or eukaryotic organism. A host or host cell comprising a nucleic acid encoding the polypeptide of the present technology is also encompassed by the present technology.

A nucleic acid may for example be DNA, RNA, or a hybrid thereof, and may also comprise (e.g., chemically) modified nucleotides, like PNA. It can be single- or double-stranded. In one embodiment, it is in the form of double-stranded DNA. For example, the nucleotide sequences of the present technology may be genomic DNA, cDNA.

The nucleic acids of the present technology can be prepared or obtained in a manner known per se, and/or can be isolated from a suitable natural source. Nucleotide sequences encoding naturally occurring (poly) peptides can for example be subjected to site-directed mutagenesis, so as to provide a nucleic acid molecule encoding polypeptide with sequence variation. Also, as will be clear to the skilled person, to prepare a nucleic acid, also several nucleotide sequences, such as at least one nucleic acid with a nucleotide sequence encoding a targeting moiety and for example a nucleic acid with a nucleotide sequence encoding one or more linkers can be linked together in a suitable manner.

Techniques for generating nucleic acids will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers.

### 5.5 Vectors

Also provided is a vector comprising the nucleic acid molecule encoding the ISVD and/or polypeptide of the present technology. A vector as used herein is a vehicle suitable for carrying genetic material into a cell. A vector includes naked nucleic acids, such as plasmids or mRNAs, or nucleic acids embedded into a bigger structure, such as liposomes or viral vectors.

In some embodiments, vectors comprise at least one nucleic acid that is optionally linked to one or more regulatory elements, such as for example one or more suitable promoter(s), enhancer(s), terminator(s), etc.). In one embodiment, the vector is an expression vector, i.e. a vector suitable for expressing an encoded polypeptide or construct under suitable conditions, e.g., when the vector is introduced into a (e.g. human) cell. DNA-based vectors include the presence of elements for transcription (e.g., a promoter and a polyA signal) and translation (e.g., Kozak sequence).

In one embodiment, in the vector, said at least one nucleic acid and said regulatory elements are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

In one embodiment, any regulatory elements of the vector are such that they are capable of providing their intended biological function in the intended host cell or host organism. For instance, a promoter, enhancer or terminator should be “operable” in the intended host cell or host organism, by which is meant that for example said promoter should be capable of initiating or otherwise controlling/regulating the transcription and/or the expression of a nucleotide sequence—e.g., a coding sequence—to which it is operably linked.

### 5.6 Compositions

The present technology also provides a composition comprising at least one ISVD of the present technology, at least one polypeptide or construct of the present technology, at least one nucleic acid molecule encoding a polypeptide of the present technology or at least one vector comprising such a nucleic acid molecule. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

### 5.7 Host Organisms

The present technology also pertains to (non-human) host cells or (non-human) host organisms capable of expressing the ISVD and/or polypeptide of the present technology, or comprising the nucleic acid encoding the ISVD and/or polypeptide of the present technology, and/or the vector comprising the nucleic acid molecule encoding the ISVD and/or polypeptide of the present technology.

Suitable host cells or host organisms are clear to the skilled person, and are for example any suitable fungal, prokaryotic or eukaryotic cell or cell line or any suitable fungal, prokaryotic or eukaryotic organism. Specific examples include HEK293 cells, CHO cells, Escherichia coli or Pichia pastoris. In one embodiment, the host is Pichia pastoris.

### 5.8 Methods and Uses of the ISVDs and/or Polypeptide

The present technology also provides a method for producing the ISVD and/or polypeptide of the present technology. The method may comprise transforming/transfecting a (non-human) host cell or host organism with a nucleic acid encoding the ISVD and/or polypeptide, expressing the ISVD and/or polypeptide in the host, optionally followed by one or more isolation and/or purification steps. In an embodiment, the method may comprise:
a) expressing, in a suitable (non-human) host organism or host cell, or in another suitable expression system, a nucleic acid sequence or genetic construct encoding the ISVD and/or polypeptide; optionally followed by:
b) isolating and/or purifying the ISVD and/or polypeptide.

In another embodiment, the method may comprise:
a) cultivating and/or maintaining a (non-human) host or host cell that is capable of expressing the ISVD and/or polypeptide of the present technology and/or that comprises a nucleic acid or a genetic construct encoding the ISVD and/or polypeptide of the present technology, under suitable circumstances that are such that said (non-human) host or host cell expresses the ISVD and/or polypeptide of the present technology;
optionally followed by:
b) isolating and/or purifying the ISVD and/or polypeptide produced.

Suitable (non-human) host organisms or host cells for production purposes will be clear to the skilled person, and may for example be any suitable fungal, prokaryotic or eukaryotic cell or cell line or any suitable fungal, prokaryotic or eukaryotic organism. Specific examples include HEK293 cells, CHO cells, Escherichia coli or Pichia pastoris. In one embodiment, the host is Pichia pastoris.

The ISVD of the present technology, the polypeptide or construct of the present technology, the nucleic acid molecule or vector as described, or the composition comprising the ISVD, polypeptide, construct, nucleic acid molecule or vector of the present technology are useful as a medicament. Accordingly, the present technology provides the ISVD of the present technology, the polypeptide or construct of the present technology, the nucleic acid molecule or vector as described, or the composition comprising the ISVD, polypeptide, construct, nucleic acid molecule or vector of the present technology for use as a medicament.

Also provided is the ISVD of the present technology, the polypeptide or construct of the present technology, the nucleic acid molecule or vector as described, or the composition comprising the ISVD, polypeptide, construct, nucleic acid molecule or vector of the present technology for use in the diagnosis, prevention and/or treatment.

Also provided is the ISVD of the present technology, the polypeptide or construct of the present technology, the nucleic acid molecule or vector as described, or the composition comprising the ISVD, polypeptide, construct, nucleic acid molecule or vector of the present technology for use in the diagnosis, prevention and/or treatment of at least one disease and/or disorder.

Also provided is the ISVD of the present technology, the polypeptide or construct of the present technology, the nucleic acid molecule or vector as described, or the composition comprising the ISVD, polypeptide, construct, nucleic acid molecule or vector of the present technology, for use in the diagnosis, prevention and/or treatment of at least one disease and/or disorder that is associated with PD-1, with its biological or pharmacological activity, and/or with the biological pathways or signalling in which PD-1 is involved.

PD-1 is known to play a role in certain proliferative diseases. Proliferative diseases are a group of conditions marked by the excessive proliferation of certain cells and turnover of cellular matrix. As such, also provided is the ISVD of the present technology, the polypeptide or construct of the present technology, the nucleic acid molecule or vector as described, or the composition comprising the ISVD, polypeptide, construct, nucleic acid molecule or vector of the present technology for use in the diagnosis, prevention and/or treatment of a proliferative disease. An example of a proliferative disease is cancer. Therefore, also provided is the ISVD of the present technology, the polypeptide or construct of the present technology, the nucleic acid molecule or vector as described, or the composition comprising the ISVD, polypeptide, construct, nucleic acid molecule or vector of the present technology for use in the diagnosis, prevention and/or treatment of cancer.

Further provided is a method for the diagnosis, prevention and/or treatment of at least one disease and/or disorder, comprising the administration, to a subject in need thereof, of a pharmaceutically active amount of at least one ISVD of the present technology, at least one polypeptide or construct of the present technology, the nucleic acid molecule or vector as described, or a composition comprising the ISVD, polypeptide, construct, nucleic acid molecule or vector of the present technology.

Further provided is a method for the diagnosis, prevention and/or treatment of at least one disease or disorder that is associated with PD-1, with its biological or pharmacological activity, and/or with the biological pathways or signalling in which PD-1 is involved, said method comprising the administration, to a subject in need thereof, of a pharmaceutically active amount of at least one ISVD of the present technology, at least one polypeptide or construct of the present technology, the nucleic acid molecule or vector as described, or a composition comprising the ISVD, polypeptide, construct, nucleic acid molecule or vector of the present technology.

Further provided is a method for the diagnosis, prevention and/or treatment of at least one proliferative disease, said method comprising the administration, to a subject in need thereof, of a pharmaceutically active amount of at least one ISVD of the present technology, at least one polypeptide or construct of the present technology, the nucleic acid molecule or vector as described, or a composition comprising the ISVD, polypeptide, construct, nucleic acid molecule or vector of the present technology.

Further provided is a method for the diagnosis, prevention and/or treatment of cancer, said method comprising the administration, to a subject in need thereof, of a pharmaceutically active amount of at least one ISVD of the present technology, at least one polypeptide or construct of the present technology, the nucleic acid molecule or vector as described, or a composition comprising the ISVD, polypeptide, construct, nucleic acid molecule or vector of the present technology.

Further provided is the use of the ISVD of the present technology, the polypeptide of the present technology, the nucleic acid molecule or vector as described, or a composition comprising the ISVD, polypeptide, construct, nucleic acid molecule or vector in the preparation of a pharmaceutical composition for the diagnosis, prevention and/or treatment of at least one disease or disorder that is associated with PD-1, with its biological or pharmacological activity, and/or with the biological pathways or signalling in which PD-1 is involved.

Further provided is the use of the ISVD of the present technology, the polypeptide of the present technology, the nucleic acid molecule or vector as described, or a composition comprising the ISVD, polypeptide, construct, nucleic acid molecule or vector in the preparation of a pharmaceutical composition for the diagnosis, prevention and/or treatment of at least one proliferative disease.

Further provided is the use of the ISVD of the present technology, the polypeptide of the present technology, the nucleic acid molecule or vector as described, or a composition comprising the ISVD, polypeptide, construct, nucleic acid molecule or vector in the preparation of a pharmaceutical composition for the diagnosis, prevention and/or treatment of cancer.

A “subject” as referred to in the context of the present technology can be any animal. In one embodiment, the subject is a mammal. Among mammals, a distinction can be made between humans and non-human mammals. Non-human animals may be for example companion animals (e.g. dogs, cats), livestock (e.g. bovine, equine, ovine, caprine, or porcine animals), or animals used generally for research purposes and/or for producing antibodies (e.g. mice, rats, rabbits, cats, dogs, goats, sheep, horses, pigs, non-human primates, such as cynomolgus monkeys, or camelids, such as llama or alpaca). In one embodiment, the subject is a human subject.

Substances, including the ISVDs, polypeptides, constructs, nucleic acid molecules and vectors of the present technology, or compositions comprising the same may be administered to a subject by any suitable route of administration, for example by parenteral administration, such as intramuscular, subcutaneous or intradermal, administration. In one embodiment, subcutaneous administration is used.

An effective amount of an ISVD, a polypeptide, a construct, a nucleic acid molecule or vector as described herein, or a composition comprising the ISVD, polypeptide, construct, nucleic acid molecule or vector can be administered to a subject in order to provide the intended diagnosis, prevention and/or treatment results.

One or more doses can be administered. If more than one dose is administered, the doses can be administered in suitable intervals in order to maximize the effect of the ISVD, polypeptide, construct, composition, nucleic acid molecule or vector.

### 5.9 Tables

Lengthy table referenced here
US20260274953A1-20260917-T00001
Please refer to the end of the specification for access instructions.

Lengthy table referenced here
US20260274953A1-20260917-T00002
Please refer to the end of the specification for access instructions.

Lengthy table referenced here
US20260274953A1-20260917-T00003
Please refer to the end of the specification for access instructions.

Lengthy table referenced here
US20260274953A1-20260917-T00004
Please refer to the end of the specification for access instructions.

Lengthy table referenced here
US20260274953A1-20260917-T00005
Please refer to the end of the specification for access instructions.

Lengthy table referenced here
US20260274953A1-20260917-T00006
Please refer to the end of the specification for access instructions.

Lengthy table referenced here
US20260274953A1-20260917-T00007
Please refer to the end of the specification for access instructions.

Lengthy table referenced here
US20260274953A1-20260917-T00008
Please refer to the end of the specification for access instructions.

Lengthy table referenced here
US20260274953A1-20260917-T00009
Please refer to the end of the specification for access instructions.

Lengthy table referenced here
US20260274953A1-20260917-T00010
Please refer to the end of the specification for access instructions.

Lengthy table referenced here
US20260274953A1-20260917-T00011
Please refer to the end of the specification for access instructions.

Lengthy table referenced here
US20260274953A1-20260917-T00012
Please refer to the end of the specification for access instructions.

Lengthy table referenced here
US20260274953A1-20260917-T00013
Please refer to the end of the specification for access instructions.

Lengthy table referenced here
US20260274953A1-20260917-T00014
Please refer to the end of the specification for access instructions.

### Multispecific Binding Proteins Targeting a TNF Receptor Superfamily (TNFRSF) Receptor and an Immune Checkpoint Protein

### Background of TNFRSFR×Immune Checkpoint Multispecific Binding Proteins

Glucocorticoid-induced TNFR-related protein (GITR; TNFRSF18) is a co-stimulatory receptor of the TNFR superfamily that is expressed on several types of immune cells of the innate and adaptive immune system. Its expression is induced on activated T cells and its interaction with GITRL induces their activation, whereas it modulates regulatory T cells (Treg) suppressive activity. However, anti-GITR antibodies have shown limited therapeutic effect in clinical trials.

Programmed death receptor 1 (PD-1) is a co-inhibitory receptor that is mainly expressed on activated T cells. Upon interaction with its ligands (PD-L1/-L2), PD-1 dampens T cell activity. Blockade of the PD-1/PD-L1 axis showed strong therapeutic impact in cancer patients, with triggering of anti-tumor immunity and long-term survival. However, the majority of patients do not respond or develop resistance.

Pre-clinical studies have shown that combination of PD-1 blockade with GITR engagement by an agonist biologic (antibody or ligand) enhances anti-tumor response in tumor-bearing mice, showing great promise for overcoming resistance to anti-PD-1/-L1 therapy.

There is a need in the art for co-targeting strategies, particularly those that are able to efficiently bind simultaneously two different cell targets. Accordingly, as described herein is a fusion binding polypeptide that comprises three or six GITR-targeting ISVDs and two PD-1-targeting ISVDs fused to the Fc portion of an IgG1.

### Summary of TNFRSFR×Immune Checkpoint Multispecifc Binding Proteins

The present disclosure is directed to, inter alia, a multispecific binding protein comprising: (a) a first binding moiety comprising at least three immunoglobulin single variable domains (ISVDs) that specifically bind a TNF receptor superfamily (TNFRSF) receptor (TNFRSF ISVDs) protein; (b) a second binding moiety comprising at least one ISVD that specifically binds to an immune checkpoint protein; and wherein the first and second binding moiety form a heterodimer.

The multispecific binding proteins of the disclosure advantageously display agonist activity to a TNFRSF protein (e.g., GITR) while also being capable of blocking activity to the immune checkpoint protein (e.g., PD-1). The multispecific binding proteins of the disclosure potently activate T cells to a higher extent than a monospecific antibody against the immune checkpoint protein (e.g., PD-1). The multispecific binding proteins of the disclosure show in vivo anti-tumor activity to a higher extent than a monospecific antibody against the immune checkpoint protein (e.g., PD-1).

In one aspect, the multispecific binding protein of disclosure comprises at least three TNFRSF ISVDs specifically bind the same or different TNFRSF protein(s). In certain aspects, the at least three TNFRSF ISVDs specifically bind the same TNFRSF protein. In certain aspects, the at least three TNFRSF ISVDs bind to the same or different contacts on the same TNFRSF protein. In certain aspects, the first binding moiety is an antagonist of the TNFRSF protein(s). In certain aspects, the first binding moiety is an agonist of the TNSFRSF protein(s).

In certain aspects, the TNFRSF protein is selected from a group consisting of: a glucocorticoid-induced tumor necrosis factor receptor-related protein (GITR), a 4-1BB, a OX40, a tumor necrosis factor receptor 1 (TNFR1), a TNFR2, a lymphotoxin beta receptor (LTBR), a CD40, a fas receptor, a CD27, a CD30, a death receptor 3 (DR3), a DR4, a DR5, a DR6, a decoy receptor 1 (DCR1), a DCR2, a DCR3, a receptor activator of nuclear factor kappa-B (RANK), an osteoprotegerin (OPG), a tumor necrosis factor-like weak inducer of apoptosis receptor (TWEAK-R), a tachykinin precursor 1 (TACI), a B cell activating factor receptor (BAFF-R), a herpes virus entry mediator (HVEM), a nerve growth factor receptor (NGFR), a B-cell maturation antigen (BCMA), a TROY, and ectodysplasin A2 receptor (EDA2R). In one aspect, the TNFRSF protein is GITR. In one aspect, the at least three TNFRSF ISVDs specifically bind to GITR.

In certain aspects, a multispecific binding protein of disclosure comprising the second binding moiety comprises at least two ISVDs. In some aspects, the at least two ISVDs specifically bind the same or different immune checkpoint protein(s). In some aspects, the at least two ISVDs bind to the same or different contacts on the same immune checkpoint protein.

In certain aspects, a multispecific binding protein of disclosure comprising the second binding moiety is an antagonist of the immune checkpoint protein(s). In one aspect, the second binding moiety is an agonist of the immune checkpoint protein(s). In certain aspects, the checkpoint protein is selected from a group consisting of: a programmed cell death protein 1 (PD-1), a PD ligand 1 (PD-L1), PD-L2, a cytotoxic T-lymphocyte-associated antigen 4 (CTLA-4), a T-cell immunoglobulin and mucin domain 3 (TIM-3), a lymphocyte activation gene 3 (LAG-3), a T cell immunoglobulin and immunoreceptor tyrosine-based inhibitory motif domain (TIGIT), a B and T lymphocyte attenuator (BTLA), a signaling lymphocytic activation molecule (SLAM), a leukocyte-associated immunoglobulin-like receptor 1 (LAIR1), a cluster of differentiation 244 (CD244), a CD28, a CD200 receptor 1 (CD200R1), a TREM-like transcript 2 (TLT2), a leukocyte immunoglobulin like receptor B4 (LILRB4), a killer cell immunoglobulin like receptor (KIR2DL2), and an inducible T-cell co-stimulator (ICOS). In one aspect, the checkpoint protein is a PD-1. In one aspect, at least two ISVDs specifically bind to PD-1 (PD-1 ISVDs).

In certain aspects, a multispecific binding protein of disclosure is bispecific or trispecific. In one aspect, the binding protein is bispecific. In certain aspects, the heterodimer comprises a fragment crystallizable (Fc) domain or variant thereof. In certain aspects, each binding moiety is operatively linked to the Fc domain or variant thereof. In certain aspects, the Fc domain or variant thereof comprises a first and a second immunoglobulin G (IgG) domain that dimerize to form the bispecific binding protein.

In certain aspects, a multispecific binding protein of disclosure comprises at least one GITR ISVD specifically binds human GITR. In certain aspects, at least one GITR ISVD is an agonist. In certain aspects, a multispecific binding protein of disclosure comprises at least three GITR ISVD that specifically bind human GITR. In certain aspects, each GITR ISVD is an agonist. In certain aspects, at least one GITR ISVD binds to an extracellular domain of a GITR subunit. In certain aspects, the at least three of the GITR ISVDs bind to the same GITR subunit. In certain aspects, at least one of the GITR ISVDs can bind to different GITR subunits.

In certain aspects, a multispecific binding protein of disclosure comprises at least three GITR ISVDs are directly linked to each other via a linker. In certain aspects, the multispecific binding protein comprises, from N-terminus to C-terminus, the at least three GITR ISVDs and a first IgG domain. In certain aspects, the multispecific binding protein comprises, from N-terminus to C-terminus, a first IgG domain and the at least three GITR ISVDs.

In certain aspects, a multispecific binding protein of the disclosure comprises at least one GITR ISVD is not directly linked to another GITR ISVD via a linker. In certain aspects of the disclosure the multispecific binding protein of the disclosure comprises at least one GITR ISVD is linked to the C-terminus and at least one GITR ISVD is linked to the N-terminus of a first IgG domain.

In certain aspects, a multispecific binding protein of the disclosure comprises a second binding moiety is linked to the N-terminus of a second IgG domain. In certain aspects, the second binding moiety is linked to the N-terminus of a second IgG domain.

In certain aspects, a multispecific binding protein of the disclosure comprises at least one GITR ISVD specific variable domain has a sequence identity of at least 80% with an amino acid sequence selected from the group consisting of SEQ ID NO: 4709 or 4711.

In certain aspects, a multispecific binding protein of the disclosure comprises at least one PD-1 ISVD specifically binds human PD-1. In certain aspects, the at least one PD-1 ISVD is an antagonist. In certain aspects, the at least one PD-1 ISVD binds to an extracellular domain of a PD-1 subunit. In certain aspects, at least two PD-1 ISVDs bind to the same PD-1 subunit. In certain aspects, at least two of the PD-1 ISVDs bind to different PD-1 subunits. In certain aspects, each PD-1 ISVD is an antagonist of PD-1.

In certain aspects of the multispecific binding protein of the disclosure, each GITR ISVD is an agonist of GITR and each PD-1 ISVD is an antagonist of PD-1.

In certain aspects, a multispecific binding protein of the disclosure comprises at least one PD-1 ISVD is not directly linked to another PD-1 ISVD via a linker. In certain aspects, the at least one PD-1 ISVD is linked to the N-terminus of a first and/or a second Fc heavy chain. In certain aspects, the at least one PD-1 ISVD is linked to the C-terminus of a first and/or a second Fc heavy chain. In certain aspects, the at least one PD-1 ISVD are linked to both the N- and the C-terminus of a first and/or second Fc heavy chain.

In certain aspects, a multispecific binding protein of the disclosure comprises at least two PD-1 ISVDs are directly linked to each other via a linker. In certain aspects, at least two PD-1 ISVDs that are linked to each other are also linked to the N-terminus of a first and/or a second Fc heavy chain. In certain aspects, at least two PD-1 ISVDs that are linked to each other are also linked to the C-terminus of a first and/or a second Fc heavy chain. In certain aspects, at least two PD-1 ISVDs are linked to both the N- and the C-terminus of a first and/or second Fc heavy chain. In certain aspects, at least two PD-1 ISVDs that are linked to each other are also linked to the N-terminus of a first Fc heavy chain. In certain aspects, the first binding moiety is linked to the N-terminus of the second Fc heavy chain. In certain aspects, at least one PD-1 ISVD is linked to the N-terminus of a first Fc heavy chain. In certain aspects, a second PD-1 ISVD is linked to the N-terminus of a second Fc heavy chain. In certain aspects, the first binding moiety is linked to the C-terminus of a first and/or second Fc heavy chain.

In certain aspects, a multispecific binding protein of the disclosure comprises at least one PD-1 ISVD specific variable domain has a sequence identity of at least 80% with an amino acid sequence selected from the group consisting of SEQ ID NO: 4710 and 4711.

In certain aspects, a multispecific binding protein of the disclosure comprises an ISVD is selected from a group consisting of: a dAb, an immunoglobulin that is suitable for use as a domain antibody (dAb), a V_{HH} (e.g., a humanized V_{HH} sequence), a camelized V_{H}, or a variant thereof. In certain aspects, a linker connects an ISVD to another ISVD is via a peptide linker. In certain aspects, the linker connecting an ISVD to the first and/or second Fc heavy chain is a peptide linker. In certain aspect, the peptide linker is at 90% identical to a peptide linker encoded by an amino acid sequence set forth in Table A-12. In certain aspects, the peptide linker is a poly-Glycine-Serine (G4S)ₙ (SEQ ID NO: 4734) linker. In certain aspects, n equals 1, 2, 3, 4, or 5.

In certain aspects, a multispecific binding protein of the disclosure comprises a Fc domain comprises a Fc modification. In certain aspects, the IgG domain is human. In certain aspects, the IgG domain is IgG1 or IgG4. In certain aspects, the Fc domain comprises an amino acid substitution at positions 234, 235, and/or 329, according to EU numbering. In certain aspects, the Fc domain comprises a leucine (L) to an alanine (A) substitution at amino acid positions 234 and 235, according to EU numbering. In certain aspects, the Fc domain comprises a proline (P) to a glycine (G) substitution at amino acid position 329, according to EU numbering. In certain aspects, the Fc domain comprising the first IgG domain comprises an amino acid substitution at position 435 and the second IgG domain comprises an amino acid mutation 436, according to EU numbering. In certain aspects, the first IgG domain comprises a histidine (H) to an arginine (R) at amino acid position 435 and the second IgG domain comprises a tyrosine (Y) to a phenylalanine (F) at amino acid position 436, according to EU numbering.

In certain aspects, a multispecific binding protein of the disclosure comprise a first and second IgG Fc domain polypeptides dimerize by knobs-into-holes interactions, Fab arm exchange (FAE), electrostatic steering interactions, hydrophobic interactions, or any combination thereof. In certain aspects, the first IgG Fc domain polypeptide comprises a knob substitution, and the second IgG Fc domain polypeptide comprises a hole substitution or wherein the first IgG Fc domain polypeptide comprises a hole substitution, and the second IgG Fc domain polypeptide comprises a knob substitution. In certain aspects, the knob substitution is selected from the group consisting of arginine (R), phenylalanine (F), tyrosine (Y), and tryptophan (W). In certain aspects, the hole substitution is selected from the group consisting of alanine (A), asparagine (N), aspartic acid (D), glycine (G), serine(S), threonine (T), and valine (V).

In certain aspects, a multispecific binding protein of the disclosure comprises a half-maximal effective concentration (EC50 [M]) in a NFkB luciferase reporter assay which is less than about 1 ×10⁻⁹. In certain aspects, a multispecific binding protein of the disclosure comprises a EC50 [M] in a T-cell activation assay which is about 1×10⁻⁹ to about 3×10⁻¹¹. In certain aspects, a multispecific binding protein of the disclosure comprises the EC50 [M] binding value for human GITR is less than about 1×10⁻¹⁰. In certain aspects, a multispecific binding protein of the disclosure comprises a half-maximal inhibitory concentration (IC50 [M]) binding value for PD-1 is less than about 1×10⁻⁹. In certain aspects, a multispecific binding protein of the disclosure comprises about 60% of the human PD-1 receptor is occupied when there is about 1×10⁻² nM or more of the multispecific binding protein. In certain aspects, a multispecific binding protein of the disclosure comprises about 60% of the human GITR receptor is occupied when there is about 1×10⁻¹ nM or more of the multispecific binding protein.

The present disclosure provides a bispecific binding protein comprising: (a) a first binding moiety comprising at least three GITR ISVDs directly linked to each other and wherein one GITR ISVD is linked to the N-terminal end of a first IgG; (b) a second binding moiety comprising at least two PD-1 ISVDs directly linked to each other and wherein one PD-1 ISVD is linked to the N-terminal end of a second IgG; and wherein each the first and second IgG1 domains heterodimerize to form a Fc domain or variant thereof.

In one aspect, a bispecific binding protein of the disclosure comprises a first binding moiety consists of at least three GITR ISVDs. In one aspect, the second moiety consists of at least two PD-1 ISVDs.

The present disclosure provides a bispecific binding protein comprising: (a) a first binding moiety comprising at least three GITR ISVDs directly linked to each other and wherein one GITR ISVD is linked to the C-terminal end of a first and a second IgG; (b) a second binding moiety comprising at least two PD-1 ISVDs wherein each PD-1 ISVD is directly linked to the N-terminal end of the first and the second IgG; and wherein each the first and second IgG1 domains form a Fc domain.

In one aspect, a bispecific binding protein of the disclosure comprises a first binding moiety consists of at least six GITR ISVDs. In one aspect, the second moiety consists of at least two PD-1 ISVDs.

In one aspect, a multispecific binding protein or a bispecific binding protein of the disclosure comprises at least one GITR ISVD comprising the three following CDR sequences:

ISVD-CDR1:
(SEQ ID NO: 4720)
GTFFSIDSMA;
ISVD-CDR2:
(SEQ ID NO: 4721)
AITGGGSPN;
and
ISVD-CDR3:
(SEQ ID NO: 4722)
EGQAGWGTALLDY.

In one aspect, a multispecific binding protein or a bispecific binding protein of the disclosure at least one PD-1 ISVD comprising one of the three following CDR sequences:

ISVD-CDR1:
(SEQ ID NO: 4723 or SEQ ID NO: 4726)
GFTLDYYVIG or GRMHSINAMA;
ISVD-CDR2:
(SEQ ID NO: 4724 or SEQ ID NO: 4727)
CLSSNGDRIN or LISWGNGITY
ISVD-CDR3:
(SEQ ID NO: 4725 or SEQ ID NO: 4728)
GTSTTVRDMCGIMYLYDY or SYGSSWYDS

In one aspect, a multispecific binding protein or a bispecific binding protein of the disclosure comprises at least one GITR ISVD specific variable domain comprising a sequence identity of at least 80% with an amino acid sequence selected from the group consisting of SEQ ID NO: 4714 and 4715.

In one aspect, a multispecific binding protein or a bispecific binding protein of the disclosure comprises at least one PD-1 ISVD specific variable domain comprising a sequence identity of at least 80% with an amino acid sequence selected from the group consisting of SEQ ID NO: 4716, 4717, 4718 and 4719.

In one aspect, a compound or construct that comprises the bispecific binding protein according to any one of the binding proteins of the disclosure that further comprises one or more other groups, residues, moieties or binding units, optionally linked via one or more peptide linker(s). In one aspect, a nucleic acid encodes the multispecific binding protein or the bispecific binding protein of the disclosure. In one aspect, an expression vector comprises a nucleic acid encodes the multispecific binding protein or the bispecific binding protein of the disclosure. In certain aspects, a host or host cell comprises a nucleic acid or an expression vector comprising a nucleic acid encoding the multispecific binding protein or the bispecific binding protein of the disclosure.

In one aspect, a pharmaceutical composition comprises the multispecific binding protein or the bispecific binding protein according to the disclosure and a pharmaceutically acceptable carrier. In one aspect, the disclosure provides a method of treating a neoplastic disorder comprising administering the multispecific binding protein or the bispecific binding protein of the disclosure to a subject. In certain aspects, administration of the binding protein results in tumor growth inhibition by 1, 2, 3, or 4-fold relative to a reference binding protein.

In one aspect, the disclosure provides a method of treating an infection comprising administering the binding protein of the disclosure to a subject.

In one aspect, the disclosure provides a method of treating a T cell, B cell, or natural killer cell mediated disease comprising administering the binding protein of the disclosure to a subject.

In one aspect, a multispecific binding protein of or a bispecific binding protein of the disclosure is for use in the treatment of neoplastic disorder. In certain aspects, the multispecific binding protein or the bispecific binding protein of the disclosure is for use in inhibiting tumor growth by 1, 2, 3, or 4-fold relative to a reference binding protein.

In one aspect, a multispecific binding protein or a bispecific binding protein of the disclosure is for use in treating an infection.

In one aspect, a multispecific binding protein or a bispecific binding protein of the disclosure is for use in treating a T cell, B cell, or natural killer cell mediated disease.

### Detailed Description of of TNFRSFR×Immune Checkpoint Multispecifc Binding Proteins

### Binding Protein or Binding Polypeptide

As used herein, the term “binding protein,” “binding polypeptide,” or “multispecific binding polypeptide or protein” or “bispecific binding polypeptide or protein” refer to a protein or polypeptide (e.g., an antibody or specific binding fragment thereof) that contains at least one binding site which is responsible for selectively binding to a target antigen of interest (e.g., a human antigen). Exemplary binding sites include an antibody variable domain, a ligand binding site of a receptor, or a receptor binding site of a ligand. In certain aspects, the binding proteins or binding polypeptides comprise multiple (e.g., two, three, four, or more) binding sites.

Native Residue vs. Modified Binding Polypeptide

As used herein, the term “native residue” refers to an amino acid residue that occurs naturally at a particular amino acid position of a binding polypeptide (e.g., an antibody or fragment thereof) and which has not been modified, introduced, or altered by the hand of man. As used herein, the term “altered binding protein,” “altered binding polypeptide,” “modified binding protein” or “modified binding polypeptide” shall refer to binding polypeptides and/or binding proteins (e.g., an antibody or fragment thereof) comprising at least one amino acid substitution, deletion and/or addition relative to the native (i.e., wild-type) amino acid sequence, and/or a mutation that results in altered glycosylation (e.g., hyperglycosylation, hypoglycosylation and/or aglycosylation) at one or more amino acid positions relative to the native (i.e., wild-type) amino acid sequence.

### Antigen and Epitope

The term “antigen” or “target antigen” as used herein refers to a molecule or a portion of a molecule that is capable of being bound by the binding site of a binding polypeptide e.g., any substance to which an antibody can be generated. A target antigen may have one or more epitopes.

The terms “epitope” and “antigenic determinant”, which can be used interchangeably, refer to the part of a macromolecule, such as a polypeptide or protein that is recognized by antigen-binding molecules, such as immunoglobulins, conventional antibodies, or immunoglobulin single variable domains, and more particularly by the antigen-binding site of said molecules. Epitopes define the minimum binding site for an immunoglobulin, and thus represent the target of specificity of an immunoglobulin. The part of an antigen-binding molecule (such as an immunoglobulin, a conventional antibody, an immunoglobulin single variable domain) that recognizes the epitope is called a “paratope”.

Examples of antigens can be a peptide, a polypeptide, a protein (e.g., a TNFRSF protein or an immune check point protein or fragment thereof), an aptamer, a polysaccharide, a sugar molecule, a carbohydrate, a lipid, an oligonucleotide, a polynucleotide, a synthetic molecule, an inorganic molecule, an organic molecule, and any combination thereof.

### Immunoglobulin Domain

The term immunoglobulin domain as used herein can refer to an immunoglobulin A, an immunoglobulin D, an immunoglobulin E, an immunoglobulin G, or an immunoglobulin M. The immunoglobulin domain can be an immunoglobulin heavy chain region or fragment thereof. In some instances, the immunoglobulin domain is from an antibody (e.g., a mammalian antibody, a recombinant antibody, a chimeric antibody, an engineered antibody, a human antibody, a humanized antibody) or an antigen binding fragment thereof. As used herein, the term “antibody” refers to such assemblies (e.g., intact antibody molecules, antibody fragments, or variants thereof) which have significant known specific immunoreactive activity to an antigen of interest (e.g., GITR and PD-1). Antibodies and immunoglobulins comprise light and heavy chains, with or without an interchain covalent linkage between them. Basic immunoglobulin structures in vertebrate systems are relatively well understood.

In certain aspects, the multispecific binding protein of the disclosure comprises an IgG domain (e.g., a human IgG domain). In certain aspects, the IgG domain is IgG1 or IgG4.

As will be discussed in more detail below, the generic term “antibody” comprises five distinct classes of antibody that can be distinguished biochemically. While all five classes of antibodies are clearly within the scope of the current disclosure, the following discussion will generally be directed to the IgG class of immunoglobulin molecules. With regard to IgG, immunoglobulins comprise two identical light chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Light chains of immunoglobulin are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin isotype subclasses (e.g., IgG1, IgG2, IgG3, IgG4, IgA1, etc.) confer functional specialization. Modified versions of each of these classes and isotypes are readily discernable to the skilled artisan in view of the instant disclosure and, accordingly, are within the scope of the current disclosure.

Both the light and heavy chains are divided into regions of structural and functional homology. The term “region” refers to a part or portion of an immunoglobulin or antibody chain and includes constant region or variable regions, as well as more discrete parts or portions of said regions. For example, light chain variable regions include “complementarity determining regions” or “CDRs” interspersed among “framework regions” or “FRs,” as defined herein.

### Constant and Variable Domains

The regions of an immunoglobulin heavy or light chain can be defined as “constant” (C) region or “variable” (V) regions, based on the relative lack of sequence variation within the regions of various class members in the case of a “constant region”, or the significant variation within the regions of various class members in the case of a “variable regions.” The terms “constant region” and “variable region” may also be used functionally. In this regard, it will be appreciated that the variable regions of an immunoglobulin or antibody determine antigen recognition and specificity. Conversely, the constant regions of an immunoglobulin or antibody confer important effector functions such as secretion, transplacental mobility, Fc receptor binding, complement binding, and the like. The subunit structures and three-dimensional configurations of the constant regions of the various immunoglobulin classes are well known.

The constant and variable regions of immunoglobulin heavy and light chains are folded into domains. The term “domain” refers to a globular region of a heavy or light chain comprising peptide loops (e.g., comprising 3 to 4 peptide loops) stabilized, for example, by β-pleated sheet and/or intrachain disulfide bond. Constant region domains on the light chain of an immunoglobulin are referred to interchangeably as “light chain constant region domains”, “CL regions” or “CL domains.” Constant domains on the heavy chain (e.g., hinge, CH1, CH2 or CH3 domains) are referred to interchangeably as “heavy chain constant region domains”, “CH” region domains or “CH domains”. Variable domains on the light chain are referred to interchangeably as “light chain variable region domains”, “V_{L} region domains or” V_{L} domains.” Variable domains on the heavy chain are referred to interchangeably as “heavy chain variable region domains”, “V_{H} region domains” or “V_{H} domains.”

By convention the numbering of the variable constant region domains increases as they become more distal from the antigen binding site or amino-terminus of the immunoglobulin or antibody. The N-terminus of each heavy and light immunoglobulin chain is a variable region and at the C-terminus is a constant region; the CH3 and CL domains actually comprise the carboxy-terminus of the heavy and light chain, respectively. Accordingly, the domains of a light chain immunoglobulin are arranged in a V_{L}-CL orientation, while the domains of the heavy chain are arranged in the V_{H}-CH1-hinge-CH2-CH3 orientation.

The assignment of amino acids to each variable region domain is in accordance with the definitions of Kabat, Sequences of Proteins of Immunological Interest (National Institutes of Health, Bethesda, MD, 1987 and 1991). Kabat also provides a widely used numbering convention (Kabat numbering) in which corresponding residues between different heavy chain variable regions or between different light chain variable regions are assigned the same number. CDRs 1, 2 and 3 of a V_{L} domain are also referred to herein, respectively, as CDR-L1, CDR-L2 and CDR-L3. CDRs 1, 2 and 3 of a V_{H} domain are also referred to herein, respectively, as CDR-H1, CDR-H2 and CDR-H3. If so noted, the assignment of CDRs can be in accordance with IMGT® (Lefranc et al., Developmental & Comparative Immunology 27:55-77; 2003) in lieu of Kabat. Numbering of the heavy chain constant region is via the EU index as set forth in Kabat (Kabat, Sequences of Proteins of Immunological Interest, National Institutes of Health, Bethesda, MD, 1987 and 1991).

V_{H} Domain and V_{L} Domain

As used herein, the term “V_{H} domain” includes the amino terminal variable domain of an immunoglobulin heavy chain, and the term “V_{L} domain” includes the amino terminal variable domain of an immunoglobulin light chain.

As used herein, the term “CH1 domain” includes the first (most amino terminal) constant region domain of an immunoglobulin heavy chain that extends, e.g., from about positions 114-223 in the Kabat numbering system (EU positions 118-215). The CH1 domain is adjacent to the V_{H} domain and amino terminal to the hinge region of an immunoglobulin heavy chain molecule and does not form a part of the Fc region of an immunoglobulin heavy chain.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al. J. Immunol. 1998, 161:4083).

### CH2 Domain

As used herein, the term “CH2 domain” includes the portion of a heavy chain immunoglobulin molecule that extends, e.g., from about positions 244-360 in the Kabat numbering system (EU positions 231-340). The CH2 domain is unique in that it is not closely paired with another domain. Rather, two N-linked branched carbohydrate chains are interposed between the two CH2 domains of an intact native IgG molecule. In certain aspects, a bispecific binding protein of the current disclosure comprises a CH2 domain derived from an IgG1 molecule (e.g., a human IgG1 molecule).

### CH3 Domain

As used herein, the term “CH3 domain” includes the portion of a heavy chain immunoglobulin molecule that extends approximately 110 residues from N-terminus of the CH2 domain, e.g., from about positions 361-476 of the Kabat numbering system (EU positions 341-445). The CH3 domain typically forms the C-terminal portion of the antibody. In some immunoglobulins, however, additional domains may extend from CH3 domain to form the C-terminal portion of the molecule (e.g., the CH4 domain in the u chain of IgM and the E chain of IgE). In certain aspects, a bispecific binding protein of the current disclosure comprises a CH3 domain derived from an IgG1 molecule (e.g., a human IgG1 molecule).

### CL Domain

As used herein, the term “CL domain” includes the constant region domain of an immunoglobulin light chain that extends, e.g., from about Kabat position 107A-216. The CL domain is adjacent to the V_{L} domain. In certain aspects, a bispecific binding protein of the current disclosure comprises a CL domain derived from a kappa light chain (e.g., a human kappa light chain).

As indicated above, the variable regions of an antibody allow it to selectively recognize and specifically bind epitopes on antigens. That is, the V_{L} domain and V_{H} domain of an antibody combine to form the variable region (Fv) that defines a three-dimensional antigen binding site. This quaternary antibody structure forms the antigen binding site present at the end of each arm of the Y. More specifically, the antigen binding site is defined by three complementary determining regions (CDRs) on each of the heavy and light chain variable regions. As used herein, the term “antigen binding site” includes a site that specifically binds (immunoreacts with) an antigen (e.g., a cell surface or soluble antigen). The antigen binding site includes an immunoglobulin heavy chain and light chain variable region and the binding site formed by these variable regions determines the specificity of the antibody. An antigen binding site is formed by variable regions that vary from one antibody to another. The altered antibodies of the current disclosure comprise at least one antigen binding site.

In certain aspects, a multispecific binding protein of the current disclosure comprises at least two different antigen binding domains that provide for the association of said binding protein with the selected antigen or target. In some aspects, the first antigen binding domain binds a TNFRSF receptor protein (e.g., TNFRSF ISVD) and the second antigen binding domain binds to an immune checkpoint protein (e.g., PD-1 ISVD). In some aspects, (a) a first binding moiety comprising at least three immunoglobulin single variable domain (ISVD) that specifically bind a TNF receptor superfamily (TNFRSF) receptor (TNFRSF ISVD); (b) a second binding moiety comprising at least one ISVD that specifically binds to an immune checkpoint protein; and wherein the first and second binding moiety form a heterodimer.

In certain aspects, the antigen binding domains are not derived from the same immunoglobulin molecule. In this regard, the variable region may be derived from any type of animal that can be induced to mount a humoral response and generate immunoglobulins against the desired antigen. As such, the variable region of a bispecific binding protein may be, for example, of mammalian origin e.g., may be human, murine, rat, goat, sheep, non-human primate (such as cynomolgus monkeys, macaques, etc.), lupine, or camelid (e.g., from camels, llamas and related species).

In naturally occurring antibodies, the six CDRs present on each monomeric antibody are short, non-contiguous sequences of amino acids that are specifically positioned to form the antigen binding site as the antibody assumes its three-dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to the immunoreactive antigen epitope.

### CDR and FR

As used herein, the term “complementarity determining region” or “CDR” refers to sequences of amino acids within antibody variable regions, which confer antigen specificity and binding affinity. In general, there are three CDRs in each heavy chain variable region (HCDR1, HCDR2, HCDR3) and three CDRs in each light chain variable region (LCDR1, LCDR2, LCDR3). “Framework regions” or “FR” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), A1-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309 (3): 657-70, (AHo numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

A “CDR” or “complementarity determining region,” or individual specified CDRs (e.g., “HCDR1,” “HCDR2,” “HCDR3”), of a given antibody or region thereof, such as a variable region thereof, should be understood to encompass a (or the specific) complementarity determining region as defined by any of the known schemes. Likewise, an “FR” or “framework region,” or individual specified FRs (e.g., “FR-H1,” “FR-H2”) of a given antibody or region thereof, such as a variable region thereof, should be understood to encompass a (or the specific) framework region as defined by any of the known schemes. In some instances, the scheme for identification of a particular CDR or FR is specified, such as the CDR as defined by the IMGT, Kabat, Chothia, AbM, or Contact method. In other cases, the particular amino acid sequence of a CDR or FR is given. Unless otherwise specified, all particular CDR amino acid sequences mentioned in the disclosure are IMGT CDRs. However, alternative CDRs defined by other schemes are also encompassed by the present disclosure, such as those determined by abYsis Key Annotation (Website: abysis.org/abysis/sequence_input/key_annotation/key_annotation.cgi).

### Antibody Variant

Exemplary multispecific binding proteins (e.g., bispecific binding proteins) can comprise antibody variants and/or specific antibody binding fragments variants. As used herein, the term “antibody variant” and “antibody specific binding fragment variant” includes synthetic and engineered forms of antibodies which are altered such that they are not naturally occurring, e.g., antibodies that comprise at least two heavy chain portions but not two complete heavy chains (such as, domain deleted antibodies or minibodies) and multispecific forms of antibodies altered to bind to two or more different antigens or to different epitopes on a single antigen); heavy chain molecules joined to scFv molecules and the like.

### Antigen Binding Fragments

Unless specifically indicated otherwise, the term “antibody,” as used herein, shall be understood to encompass antibody molecules comprising two immunoglobulin heavy chains and two immunoglobulin light chains (i.e., “full antibody molecules”) as well as antigen binding fragments thereof. Other engineered molecules, such as domain specific binding proteins, single domain binding proteins, domain deleted binding proteins, chimeric binding proteins, CDR grafted binding proteins, diabodies, triabodies, tetrabodies, minibodies, immunoglobulin single variable domains (ISVDs) (e.g., monovalent ISVDs, bivalent ISVDs, etc.), small modular immunopharmaceuticals (SMIPs), and shark variable IgNAR domains, are also encompassed within the expression “antigen binding fragment,” as used herein. The term “multispecific antibody” denotes a binding fragment or derivative thereof that combines the antigen-binding sites of two or more antibodies within a single molecule. The terms “antigen binding portion”, “antigen binding fragment”, “binding protein” or “binding moiety” and the like, as used herein, include any naturally occurring, enzymatically obtainable, synthetic, or genetically engineered polypeptide or glycoprotein that specifically binds to at least one target antigen to form a complex.

In certain aspects, the term “antigen binding fragment” refers to a polypeptide fragment of a multispecific (e.g., a bispecific) binding protein. Antigen binding fragments of a multispecific binding protein, or a binding fragment or derivative thereof can be derived, e.g., from full multispecific binding protein molecules using any suitable standard techniques such as proteolytic digestion or recombinant genetic engineering techniques involving the manipulation and expression of DNA encoding multispecific binding protein, or a binding fragment or derivative thereof variable and (optionally) constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add, or delete amino acids, etc.

### Multispecific

As used herein, a “multispecific” binding protein is a binding protein that specifically binds two or more types of antigens.

A multispecific binding protein that binds two antigens, and/or two different epitopes of different antigens, is also referred to herein as a “bispecific” binding protein (e.g., a multispecific binding protein that binds GITR and an immune checkpoint protein, such as PD-1). In some aspects, the multispecific binding of the disclosure is a bispecific binding protein. In some aspects, the bispecific binding protein of the disclosure comprises (a) a first binding moiety comprising at least three immunoglobulin single variable domains (ISVDs) that specifically bind a TNF receptor superfamily (TNFRSF) receptor (TNFRSF ISVDs) protein; (b) a second binding moiety comprising at least one ISVD that specifically binds to an immune checkpoint protein; and wherein the first and second binding moiety form a heterodimer. A multispecific binding protein that binds three antigens, and/or three different epitopes, is also referred to herein as a “trispecific” binding protein. In some aspects, the multispecific binding of the disclosure is a trispecific binding protein.

Thus, the multispecific binding protein is able to bind two or more different targets simultaneously, for example, GITR and an immune checkpoint protein, such as PD-1. Genetic engineering can be used to design, modify, and produce the multispecific binding protein, or a binding fragment or derivative thereof with a desired set of binding properties and effector functions.

In some aspects, a multispecific binding protein of the disclosure comprises a V_{H}, a Fab, a Fab′, a F(ab′)₂, a variable fragment (Fv), a disulfide-stabilized Fv (dsFv), a single-chain Fv (scFv), a diabody, a triabody, an immunoglobulin single variable domain (ISVD), or an AFFIBODY®. In some aspects, wherein the multispecific binding protein further comprises a Fc domain or a variant thereof. In one aspect, the ISVD is a V_{HH}, humanized V_{HH}, a camelized V_{H}, a (single) domain antibody (such as a dAb or a sdAb), or a VNAR. In some aspects, the scFV is a linear scFV or a tandem scFV.

Techniques for making multispecific binding proteins (e.g., multispecific antibodies) include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A. et al., EMBO J. 10 (1991) 3655-3659), and “knob-in-hole” engineering (see, e.g., U.S. Pat. No. 5,731,168). In some embodiments, the bispecific antibodies are prepared using a macro-assembly technique using two half-antibodies, e.g., as described in Spiess, C., Merchant, M., Huang, A. et al. Bispecific antibodies with natural architecture produced by co-culture of bacteria expressing two distinct half-antibodies. Nat Biotechnol 31, 753-758 (2013).

Multispecific antibodies may also be made by engineering electrostatic steering effects for making binding protein Fc-heterodimeric molecules (WO 2009/089004); cross-linking two or more antibodies or fragments (see, e.g., U.S. Pat. No. 4,676,980, and Brennan, M. et al., Science 229 (1985) 81-83); using leucine zippers to produce bi-specific antibodies (see, e.g., Kostelny, S. A. et al., J. Immunol. 148 (1992) 1547-1553; using “diabody” technology for making multispecific binding protein fragments (see, e.g., Holliger, P. et al., Proc. Natl. Acad. Sci. USA 90 (1993) 6444-6448); and using single-chain Fv (scFv) dimers (see, e.g., Gruber, M et al., J. Immunol. 152 (1994) 5368-5374); and preparing trispecific binding proteins as described, e.g., in Tutt, A. et al., J. Immunol. 147 (1991) 60-69).

A wide variety of recombinant multispecific binding protein formats have been developed, e.g., by fusion an IgG binding protein format and single chain domains (see Kontermann R E, mAbs 4:2, (2012) 1-16). Multispecific binding proteins wherein the variable domains V_{L} and V_{H} or the constant domains CL and CH1 are replaced by each other are described in WO2009080251 and WO2009080252.

In one aspect, a multispecific binding protein a first and a second IgG Fc domain polypeptide of a multispecific binding protein can dimerize. In some aspects, a multispecific binding protein of the disclosure dimerize by knobs-into-holes interactions, Fab arm exchange (FAE), electrostatic steering interactions, hydrophobic interactions, or any combination thereof.

### Multispecific Binding Protein Targeting a TNFRSFR and an Immune Checkpoint Protein

### Binding Moiety

The term “binding moiety” is used herein in the broadest sense to encompass any chemical entity capable of specific binding to a target, such as an antigen (e.g., GITR or PD-1). The terms “binding moiety” and “binding domain” can be used interchangeably herein.

Examples of a binding moiety of the multispecific binding protein described herein can comprise an antibody, a V_{H}, a Fab, a Fab′, a F(ab′)2, a variable fragment (Fv), a disulfide-stabilized Fv (dsFv), a single-chain Fv (scFv), a diabody, a triabody, an immunoglobulin single variable domain (ISVD), or an AFFIBODY®. Other binding moiety examples include a Fab fragment, a F(ab′)2 fragment, an Fv fragment a fragment containing a complementarity determining region (CDR), an isolated CDR, or other suitable fragment.

In certain aspects, a first cell surface binding moiety and a second binding moiety of a multispecific binding protein each independently comprise an antigen binding fragment that comprises at least one variable domain of an antibody or an antigen binding fragment. At least one constant domain can optionally be covalently linked to one or both the first and second binding moieties. The variable domain(s) can be of any size or amino acid composition and will generally comprise at least one CDR, which is adjacent to or in frame with one or more framework sequences. In antigen binding fragments having a V_{H} domain associated with a V_{L} domain, the V_{H} and V_{L} domains can be situated relative to one another in any suitable arrangement. For example, the variable region can be dimeric and contain V_{H} V_{H} , V_{H} V_{L} or V_{L}-V_{L} dimers. Alternatively, the antigen binding fragment can contain a monomeric V_{H} or V_{L} domain.

Non limiting, exemplary configurations of variable and constant domains that can be found within an antigen binding fragment include: (i) V_{H} CH1; (ii) V_{H} CH2; (iii) V_{H} CH3; (iv) V_{H} CH1 CH2; (v) V_{H} CH1 CH2 CH3; (vi) V_{H} CH2 CH3; (vii) V_{H} CL; (viii) V_{L} CH1; (ix) V_{L} CH2; (X) V_{L} CH3; (xi) V_{L} CH1 CH2; (xii) V_{L} CH1 CH2 CH3; (xiii) V_{L} CH2 CH3; and (xiv) V_{L} CL. In any configuration of variable and constant domains, including any of the exemplary configurations listed above, the variable and constant domains can be either directly linked to one another or can be linked by a full or partial hinge or linker region. A hinge region can comprise of at least 2 (e.g., 5, 10, 15, 20, 40, 60 or more) amino acids, which result in a flexible or semi flexible linkage between adjacent variable and/or constant domains in a single polypeptide molecule. Moreover, the first and second binding moieties of this disclosure can comprise a homo dimer or hetero dimer (or other multimer) of any of the variable and constant domain configurations listed above in non-covalent association with one another and/or with one or more monomeric V_{H} or V_{L} domain (e.g., by disulfide bond(s)).

### Fab, F(ab′)₂, Fab′

The term “Fab” denotes a binding protein or a binding fragment thereof having a molecular weight of about 50,000 Da and antigen binding activity, in which about a half of the N-terminal side of H chain and the entire L chain, among fragments obtained by treating IgG with a protease, papain, are bound together through a disulfide bond.

The term F(ab′)₂ refers to a binding protein or a binding fragment thereof having a molecular weight of about 100,000 Da and antigen binding activity, which is slightly larger than a Fab bound via a disulfide bond of the hinge region, among fragments obtained by treating IgG with a protease, pepsin.

The term Fab′ refers to a binding protein or a binding fragment having a molecular weight of about 50,000 Da and antigen binding activity, which is obtained by cutting a disulfide bond of the hinge region of the F(ab′)2.

scFV

A single chain Fv (“scFv”) polypeptide is a covalently linked V_{H}:V_{L} heterodimer that is usually expressed from a gene fusion including V_{H} and V_{L} encoding genes linked by a peptide-encoding linker. A human scFv fragment includes CDRs that are held in appropriate conformation by, e.g., using gene recombination techniques. Divalent and multivalent multi-specific binding proteins, or binding fragments or derivatives thereof can form either spontaneously by association of monovalent scFvs, or can be generated by coupling monovalent scFvs by a peptide linker, such as divalent sc(Fv)2. A “dsFv” is a V_{H}:V_{L} heterodimer stabilized by a disulfide bond. “(dsFv)2” denotes two dsFv coupled by a peptide linker.

### Immunoglobulin Single Variable Domain (ISVD)

The term “immunoglobulin single variable domain (ISVD)” and “single variable domain” in the context of the multispecific binding proteins described herein (e.g., the GITR×PD-1 bispecific binding proteins) are the same as applied above in Section 5.2 (immunoglobulin single variable domains).

In Vivo Half-Life and ISVD that Increase In Vivo Half-Life

In certain aspects, a multispecific binding protein further comprises one or more groups, residues, moieties or binding units provide the multispecific binding protein with enhanced therapeutic efficacy. For example, therapeutic efficacy can be enhanced by increasing the in vivo half-life (referred to also as “half-life”) of the multispecific binding protein described herein. In certain aspects, a multispecific binding protein further comprising one or more binding moieties operatively linked to the multispecific binding protein by a linker region, e.g., a linker encoded by any one of the amino acid sequences set forth in Table A-12.

The term “in vivo half-life extension” means that the multispecific binding protein has an increased half-life in a mammal, such as a human subject, after administration. Half-life can be expressed, e.g., as t1/2beta. A multispecific binding protein half-life can be extended e.g., a polyethylene glycol molecule, an Fc domain, a modified Fc domain, serum proteins or fragments thereof, serum binding proteins (e.g., an IgG), or binding moieties that can bind to serum proteins (e.g., an ISVD that binds a serum protein). A multispecific binding protein half-life can be extended e.g., by one or more binding units that facilitates binding to a FcRn.

In certain aspects, the binding unit that facilitates binding to a FcRn is selected from a group consisting of: a Fc constant domain, a modified Fc domain, a serum albumin, a serum albumin, a serum immunoglobulin, or a variant thereof. In certain aspects, the serum albumin is a human serum albumin or the serum immunoglobulin is an IgG.

In certain aspects, the binding unit is an ISVD that binds to a human serum albumin e.g., a ISVD that binds to a human serum albumin described in WO 2004/041865, WO 2006/122787, WO 2006/122787, WO2012/175400, WO 2012/175741, WO 2015/173325, WO 2017/080850, WO 2017/085172, WO 2018/104444, WO 2018/134235, or WO 2018/134234, which are incorporated by reference herein in their entirety.

In certain aspects, an ISVD that binds to a human serum albumin is linked to the first or second binding moieties of the multispecific binding protein described herein. In certain aspects, the ISVD that binds to a human serum albumin extends the half-life of the multispecific binding protein relative to a reference multispecific binding protein that does not have said ISVD.

In certain aspects, an ISVD binding to human serum albumin is linked to the C-terminal end of the multispecific binding protein so that the C-terminal end of the human serum albumin is an exposed C-terminal end. In certain aspects, the C-terminal end of the ISVD binding to human serum albumin comprises a C-terminal extender. In certain aspects, the C-terminal extender is a C-terminal alanine (A) or glycine (G) extension. In certain aspects, C-terminal alanine (A) or glycine (G) extension comprises 1-3 alanine or glycine residues, respectively.

In certain aspects, an ISVD capable of binding to human serum albumin is not linked to the C-terminal end of the multispecific binding protein and the C-terminal end of the human serum albumin is not an exposed C-terminal end. In certain aspects, the C-terminal sequence “VTVSS” (SEQ ID NO: 4713) is directly followed by a linker region, e.g., a linker region of Table A-12.

### Linker Region

As used herein, the term “linker” denotes a peptide that fuses together two or more ISVDs into a single molecule. The use of linkers to connect two or more (poly) peptides is well known in the art. Further exemplary peptidic linkers are shown in Table A-12. One often used class of peptidic linker are known as the “Gly-Ser” or “GS” linkers. These are linkers that essentially consist of glycine (G) and serine(S) residues, and usually comprise one or more repeats of a peptide motif such as the GGGGS (SEQ ID NO: 4795) motif (for example, having the formula (Gly-Gly-Gly-Gly-Ser)ₙ (SEQ ID NO: 4795) in which n may be 1, 2, 3, 4, 5, 6, 7 or more). Some often-used examples of such GS linkers are 9GS linkers (GGGGSGGGS, SEQ ID NO: 2423), 15GS linkers (n=3) and 35GS linkers (n=7). Reference is for example made to Chen et al. 2013 (Adv. Drug Deliv. Rev. 65 (10): 1357-1369) and Klein et al. 2014 (Protein Eng. Des. Sel. 27 (10): 325-330).

In certain aspects, a multispecific binding protein of the disclosure comprises the linker connecting an ISVD to another ISVD is via a peptide linker. In certain aspects, the linker also connecting an ISVD to the first and/or second Fc heavy chain is a peptide linker. In certain aspects, the peptide linker is a poly-Glycine-Serine (G₄S)ₙ (SEQ ID NO: 4734) linker. In certain aspects, n equals 1, 2, 3, 4, or 5.

In certain aspects, the peptide linker comprises an amino acid sequence which is at least 75, 80, 85, 90, 93, 95, 99% or more identical to an amino acid sequence of Table A-12.

In certain aspects, the linker comprises at least a portion of a hinge region (e.g., derived from an IgG1, IgG2, IgG3, or IgG4 molecule) and a series of glycine-serine amino acid residues.

In certain aspects, a multispecific binding protein comprises a second binding moiety which is operatively linked to a first cell surface binding moiety and that specifically binds to the target protein.

In certain aspects, a multispecific binding protein further comprises one or more binding moieties operatively linked to the first cell surface binding moiety or the second binding moiety that specifically binds to the target protein. In certain aspects, the operative linker is a peptide linker. In certain aspects, the peptide linker is at 90% identical to a peptide linker encoded by an amino acid sequence set forth in SEQ ID NOs: 2418-2434 or 4729-4734.

In certain aspects, a compound or construct comprises the multispecific or the bispecific binding protein according to the disclosure that further comprises one or more other groups, residues, moieties or binding units, optionally linked via one or more peptide linker(s).

In certain aspects, a multispecific binding protein comprises at least three GITR ISVDs are directly linked to each other via a linker. In certain aspects, a multispecific binding protein of the disclosure comprises from N-terminus to C-terminus, the at least three GITR ISVDs and a first IgG domain. In some aspects, a multispecific binding protein of the disclosure comprises from N-terminus to C-terminus, a first IgG domain and the at least three GITR ISVDs.

In certain aspects, at least one GITR ISVD is not directly linked to another GITR ISVD via a linker. In certain aspects, at least one GITR ISVD is linked to the C-terminus and at least one GITR ISVD is linked to the N-terminus of a first IgG domain.

In certain aspects, the at least three GITR ISVDs that are linked to each other are also linked to the N-terminus of a first and/or a second Fc heavy chain. In certain aspects, the at least three GITR ISVDs that are linked to each other are also linked to the C-terminus of the first and/or second Fc heavy chain. In certain aspects, at least one GITR ISVD is linked to the C-terminus and at least one other GITR ISVD is linked to the N-terminus end of a first and/or second Fc heavy chain.

In certain aspects, a multispecific binding protein of disclosure comprises at least three GITR ISVDs that are linked to each other are also linked to the N-terminus of the first Fc heavy chain and wherein the second binding moiety is linked to the N-terminus of the second Fc heavy chain.

In certain aspects, a multispecific binding protein of disclosure comprises at least three GITR ISVDs that are linked to each other and are also linked to the C-terminus of the first Fc heavy chain.

In certain aspects, at least three GITR ISVDs that are linked to each other are also linked to the C-terminus of the second Fc heavy chain. In certain aspects, the second binding moiety is linked to the N-terminus of the first and/or second Fc heavy chain.

In certain aspects, a multispecific binding protein of disclosure comprises at least one PD-1 ISVD that is not directly linked to another PD-1 ISVD via a linker. In certain aspects, the at least one PD-1 ISVD is linked to the N-terminus of a first and/or a second Fc heavy chain. In certain aspects, the at least one PD-1 ISVD is linked to the C-terminus of a first and/or a second Fc heavy chain. In certain aspects, the at least one PD-1 ISVD are linked to both the N- and the C-terminus of a first and/or second Fc heavy chain.

In certain aspects, a multispecific binding protein of disclosure comprises at least two PD-1 ISVDs are directly linked to each other via a linker. In certain aspects, the at least two PD-1 ISVDs that are linked to each other are also linked to the N-terminus of a first and/or a second Fc heavy chain. In certain aspects, the at least two PD-1 ISVDs that are linked to each other are also linked to the C-terminus of a first and/or a second Fc heavy chain. In certain aspects, the at least two PD-1 ISVDs are linked to both the N- and the C-terminus of a first and/or second Fc heavy chain.

In certain aspects, a multispecific binding protein of disclosure comprises at least two PD-1 ISVDs that are linked to each other are also linked to the N-terminus of a first Fc heavy chain. In certain aspects, the first binding moiety is linked to the N-terminus of the second Fc heavy chain. In certain aspects, the at least one PD-1 ISVD is linked to the N-terminus of a first Fc heavy chain and a second PD-1 ISVD is linked to the N-terminus of a second Fc heavy chain. In certain aspects, the first binding moiety is linked to the C-terminus of a first and/or second Fc heavy chain.

### Increased Half-Life

In certain aspects, a multispecific binding protein further comprising the one or more binding moieties provide the multispecific binding protein with increased half-life, compared to a reference multispecific binding protein without said binding moieties. In certain aspects, the one or more binding units facilitates binding to a FcRn. In certain aspects, the binding unit that facilitates binding to a FcRn is selected from a group consisting of: a Fc constant domain, a modified Fc domain, a serum albumin, a serum albumin, a serum immunoglobulin, or a variant thereof. In certain aspects, the serum albumin is a human serum albumin or the serum immunoglobulin is an IgG.

### Fc Domain

In some aspects, the multispecific binding protein can comprise a Fc domain. As used herein, the term “Fc domain” or “Fc region” of “Fc heavy chain” (used interchangeably) is defined as the portion of a heavy chain constant region beginning in the hinge region just upstream of the papain cleavage site (i.e., residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The terms “Fc variant,” “modified Fc,” “engineered Fc” are interchangeably used herein refer to a molecule or sequence that is modified from a native Fc but still comprises a binding site for the salvage receptor, FcRn (neonatal Fc receptor). Exemplary Fc variants, and their interaction with the salvage receptor, are known in the art. A modified Fc domain can comprise a molecule or sequence that is humanized from a non-human native Fc. Furthermore, a native Fc comprises regions that can be removed because they provide structural features or biological activity that are not required for the multispecific binding protein compositions. Thus, the term “modified Fc domain” comprises a molecule or sequence that lacks one or more native Fc sites or residues, or in which one or more Fc sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

“Effector function” as used herein is meant a biochemical event that results from the interaction of an antibody Fc region with an Fc receptor or ligand. A “functional Fc region” possesses an “effector function” of a native sequence Fc region. Exemplary “effector functions” include antibody-dependent cell-mediated cytotoxicity (ADCC) or antibody-dependent cell-mediated phagocytosis (ADCP).

The term “EU index” refers to the EU numbering convention for the constant regions of an antibody, as described in Edelman, G M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety. Unless otherwise stated, all antibody Fc region numbering employed herein corresponds to the EU numbering scheme, as described in Edelman et al. (Proc. Natl. Acad. Sci. 63(1): 78-85. 1969).

The Fc domain of the multispecific binding protein of the disclosure may be engineered to promote heterodimerization over homodimerization. For example, the heavy chain constant region of the first heavy-light chain pair may comprise a different amino acid sequence from the heavy chain constant region of the second heavy-light chain pair, wherein the different amino acid sequences are engineered to promote heterodimerization of the heavy chain constant regions. Examples include knobs-into-holes mutations or charge pair mutations. Alternatively, the heavy chain constant region of the first heavy-light chain pair may be identical to the heavy chain constant region of the second heavy-light chain pair, in which case it is expected that both homodimers and heterodimers will assemble, and these will be subsequently separated using one or more purification steps in the binding protein manufacturing process to isolate the desired heterodimer.

In certain aspects, a first and/or second Fc constant domain is derived from an immunoglobulin class selected from a group consisting of: IgM, IgG, IgD, IgA, IgE. Chimeric Fc domains comprising portions of Fc domains from different species or Ig classes can also be employed. In certain aspects, a Fc constant domain is an IgG Fc domain, an IgG1 Fc domain, or an IgG4 Fc domain.

In certain aspects, the first Fc domain can be engineered to pair or heterodimerize with the second Fc domain. For example, a multispecific binding protein of the disclosure may comprise a first polypeptide comprising a first binding moiety comprising at least one immunoglobulin single variable domain (ISVD) that specifically binds glucocorticoid-induced TNFR family-related receptor (GITR) (GITR ISVD) and an Fc domain and a second polypeptide comprising a second binding moiety comprising at least one ISVD that specifically binds to an immune checkpoint protein, wherein the second polypeptide comprises a second Fc domain capable of dimerizing with the first Fc domain. In certain aspects the first and/or second Fc domain can be modified.

In certain aspects, a multispecific binding protein of the disclosure comprise a fragment crystallizable (Fc) domain or variant thereof. In certain aspects, each binding moiety (e.g., the first and the second binding moiety) is operatively linked to the Fc domain or variant thereof. In certain aspects, the Fc domain or variant thereof comprises a first and a second immunoglobulin G (IgG) domain that dimerize to form the bispecific binding protein.

### Fc Mutations

In certain aspects, a multispecific binding protein of the disclosure comprises a modified Fc domain and can further comprise one or more, e.g., two or more, three or more, or four or more, amino acid substitutions that confers a multispecific binding protein with one or more biochemical characteristics other than glycan modification.

Exemplary modified Fc domain amino acid substitutions that can confer additional biochemical characteristics to the multispecific binding proteins described herein are disclosed in WO2021016571A2, which is incorporated by reference in its entirety.

In certain aspects, a modified Fc region of a multispecific binding protein of the current disclosure comprises one or more mutations to modulate half-life (See e.g., Dall'Acqua et al. (2006) J Biol Chem 281:23514-24, Zalevsky et al. (2010) Nat Biotechnol 28:157-9, Hinton et al. (2004) J Biol Chem 279:6213-6, Hinton et al. (2006) J Immunol 176:346-56, Shields et al. (2001) J Biol Chem 276:6591-604, Petkova et al. (2006) Int Immunol 18:1759-69, Datta-Mannan et al. (2007) Drug Metab Dispos 35:86-94, Vaccaro et al. (2005) Nat Biotechnol 23:1283-8, Yeung et al. (2010) Cancer Res 70:3269-77 and Kim et al. (1999) Eur J Immunol 29:2819-25. (e.g., T250Q, M252Y, I253A, S254T, T256E, P2571, T307A, D376V, E380A, M428L, H433K, N434S, N434A, N434H, N434F, H435A and/or H435R).

In certain aspects, a modified Fc region of a multispecific binding protein of the current disclosure can have enhanced FcRn binding affinities at both an acidic pH (e.g., less than about 7.0, no more than about 6.5, or no more than about 6.0) and a non-acidic pH (e.g., no less than about 7.0, or no less than about 7.4), as compared to its wild-type. In another example, a multispecific binding protein comprising a modified Fc can comprise one or more amino acid mutations (e.g., substitutions) which alter the effector functions (e.g., ADCC or CDC function) of the Fc domain, as compared to a corresponding wild-type molecule, e.g., a molecule having the same structure as the FcRn antagonist except that it has a wild-type Fc domain. In another example, a multispecific binding protein can comprise a modified Fc domain comprising one or more amino acid mutations (e.g., substitutions) which alter (e.g., increase or decrease) the circulating half-life (e.g., serum half-life) of the FcRn antagonist, as compared to the corresponding wild-type molecule.

In certain aspects, a multispecific binding protein described herein can comprise a modified Fc domain that alters serum half-life compared to a multispecific binding protein comprising a wild-type Fc domain. In certain aspects, a modified Fc domain has an increased serum half-life compared to a multispecific binding protein comprising a wild-type Fc domain. In certain aspects, a Fc domain is modified to alter FcRn binding affinity compared to a multispecific binding protein comprising a wild-type Fc domain. In certain aspects, a modified Fc domain has enhanced FcRn binding affinity compared to a multispecific binding protein comprising a wild-type Fc domain. In certain aspects, a Fc domain is modified to enhance the FcRn binding affinity at an acidic pH compared to a multispecific binding protein comprising a wild-type Fc domain.

In certain aspects, a multispecific binding protein can have a modified Fc domain. In certain aspects, a multispecific binding protein can have a tyrosine (Y) at amino acid position 252, according to EU numbering. In certain aspects, a multispecific binding protein can have an aspartic acid (D) or a glutamic acid (E) at amino acid position 256, according to EU numbering. In certain aspects, a multispecific binding protein can have a tryptophan (W) or a glutamine (Q) at amino acid position 307, according to EU numbering. In certain aspects, a multispecific binding protein can have a phenylalanine (F) or a tyrosine (Y) at amino acid position 434; according to EU numbering.

In certain aspects, a multispecific binding protein can have a modified Fc domain comprising any combination of the following four amino acid residues: a tyrosine (Y) at amino acid position 252, an aspartic acid (D) or a glutamic acid (E) at amino acid position 256, a tryptophan (W) or a glutamine (Q) at amino acid position 307, and a phenylalanine (F) or a tyrosine (Y) at amino acid position 434; according to EU numbering.

In certain aspects, a multispecific binding protein can comprise a modified Fc domain having a combination of amino acid residues selected from the group consisting of: a) a tyrosine (Y) at amino acid position 252, an aspartic acid (D) at amino acid position 256, a glutamine (Q) at amino acid position 307, and a tyrosine (Y) at amino acid position 434; b) a tyrosine (Y) at amino acid position 252, a glutamic acid (E) at amino acid position 256, a tryptophan (W) at amino acid position 307, and a tyrosine (Y) at amino acid position 434; c) a tyrosine (Y) at amino acid position 252, a glutamic acid (E) at amino acid position 256, a glutamine (Q) at amino acid position 307, and a tyrosine (Y) at amino acid position 434; d) a tyrosine (Y) at amino acid position 252, an aspartic acid (D) at amino acid position 256, a glutamine (Q) at amino acid position 307, and a phenylalanine (F) at amino acid position 434; e) a tyrosine (Y) at amino acid position 252, an aspartic acid (D) at amino acid position 256, a tryptophan (W) at amino acid position 307, and a tyrosine (Y) at amino acid position 434; and f) a tyrosine (Y) at amino acid position 252, an aspartic acid (D) at amino acid position 256, a tryptophan (W) at amino acid position 307, and a phenylalanine (F) at amino acid position 434; according to EU numbering.

In certain aspects, a multispecific binding protein can comprise a modified Fc domain comprising a quadruple amino acid substitution selected from the group consisting of: M252Y/T256D/T307Q/N434Y, M252Y/T256E/T307W/N434Y, M252Y/T256E/T307Q/N434Y, M252Y/T256D/T307Q/N434F, M252Y/T256D/T307W/N434Y, and M252Y/T256D/T307W/N434F; according to EU numbering.

In certain aspects, a multispecific binding protein of the disclosure can comprise a modified Fc domain comprising: an amino acid substitution at positions 234, 235, and/or 329, according to EU numbering. In certain aspects, the Fc domain comprises a leucine (L) to an alanine (A) substitution at amino acid positions 234 and 235, according to EU numbering. In certain aspects, the Fc domain comprises a proline (P) to a glycine (G) substitution at amino acid position 329, according to EU numbering.

In certain aspects, a multispecific binding protein of the disclosure can comprise a modified Fc domain comprising: the first IgG domain comprises an amino acid substitution at position 435 and the second IgG domain comprises an amino acid mutation 436, according to EU numbering. In certain aspects, the first IgG domain comprises a histidine (H) to an arginine (R) at amino acid position 435 and the second IgG domain comprises a tyrosine (Y) to a phenylalanine (F) at amino acid position 436, according to EU numbering.

### Knobs and Holes

Techniques for making multispecific binding proteins (e.g., multispecific antibodies) include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A. et al., EMBO J. 10 (1991) 3655-3659), and “knob-in-hole” engineering (see, e.g., U.S. Pat. No. 5,731,168). Multispecific binding proteins may also be made by engineering electrostatic steering effects for making binding protein Fc-heterodimeric molecules (WO 2009/089004); cross-linking two or more antibodies or fragments (see, e.g., U.S. Pat. No. 4,676,980, and Brennan, M. et al., Science 229 (1985) 81-83); using leucine zippers to produce bi-specific antibodies (see, e.g., Kostelny, S. A. et al., J. Immunol. 148 (1992) 1547-1553; using “diabody” technology for making multispecific binding protein fragments (see, e.g., Holliger, P. et al., Proc. Natl. Acad. Sci. USA 90 (1993) 6444-6448); and using single-chain Fv (scFv) dimers (see, e.g., Gruber, M et al., J. Immunol. 152 (1994) 5368-5374); and preparing trispecific binding proteins as described, e.g., in Tutt, A. et al., J. Immunol. 147 (1991) 60-69).

A wide variety of recombinant multispecific binding protein formats have been developed, e.g., by fusion of, e.g., an IgG binding protein format and single chain domains (see Kontermann R E, mAbs 4:2, (2012) 1-16). Multispecific binding proteins wherein the variable domains V_{L} and V_{H} or the constant domains CL and CH1 are replaced by each other are described in WO2009080251 and WO2009080252.

An approach to circumvent the problem of mispaired byproducts, is known as “knobs-into-holes”, aims at forcing the pairing of two different binding protein heavy chains by introducing mutations into the CH3 domains to modify the contact interface. On one chain bulky amino acids can be replaced by amino acids with short side chains to create a “hole.” Conversely, amino acids with large side chains can be introduced into the other CH3 domain, to create a “knob.” By co-expressing these two heavy chains (and two identical light chains, which have to be appropriate for both heavy chains), high yields of heterodimer formation (“knob-hole”) versus homodimer formation (“hole-hole” or “knob-knob”) was observed (Ridgway J B, Presta L G, Carter P; and WO1996027011). The percentage of heterodimer could be further increased by remodeling the interaction surfaces of the two CH3 domains using a phage display approach and the introduction of a disulfide bridge to stabilize the heterodimers (Merchant A. M, et al, Nature Biotech 16 (1998) 677-681; Atwell S, Ridgway J B, Wells J A, Carter P., J Mol Biol 270 (1997) 26-35). New approaches for the knobs-into-holes technology are described in e.g., in EP 1870459A1. Xie, Z., et al, J Immunol Methods 286 (2005) 95-101 refers to a format of multispecific binding protein using scFvs in combination with knobs-into-holes technology for the Fc part.

In certain aspects, the CH3 domains of the heavy chains of a multispecific binding protein can be altered by the “knob-into-holes” technology, which is described in detail with several examples in e.g., WO 96/027011, WO 98/050431, Ridgway J. B. et al., Protein Eng. 9 (1996) 617-621, Merchant A. M. et al., Nat Biotechnol 16 (1998) 677-681. In this method, the interaction surfaces of the two CH3 domains are altered to increase the heterodimerization of both heavy chains containing said two CH3 domains. Each of the two CH3 domains (of the two heavy chains) can be the “knob,” while the other is the “hole.” The introduction of a disulfide bridge can be utilized to stabilize the heterodimers (Merchant A. M et al., Nature Biotech 16 (1998) 677-681, Atwell, S. et al., J. Mol. Biol. 270 (1997) 26-35), as well as to increase the yield.

The Fc domain of a multispecific binding protein can be engineered to promote heterodimerization over homodimerization. For example, the heavy chain constant region of the first heavy-light chain pair can comprise a different amino acid sequence from the heavy chain constant region of the second heavy-light chain pair, wherein the different amino acid sequences are engineered to promote heterodimerization of the heavy chain constant regions. Examples include knobs-into-holes mutations or charge pair mutations. Alternatively, the heavy chain constant region of the first heavy-light chain pair may be identical to the heavy chain constant region of the second heavy-light chain pair, in which case it is expected that both homodimers and heterodimers will assemble, and these will be subsequently separated using one or more purification steps in the antibody manufacturing process to isolate the desired heterodimer.

In certain aspects, a multispecific binding protein of the disclosure comprises a first and a second IgG Fc domain polypeptide that dimerize to form the bispecific binding protein. In certain aspects, the first and second IgG Fc domain polypeptides dimerize by knobs-into-holes interactions, Fab arm exchange (FAE), electrostatic steering interactions, hydrophobic interactions, or any combination thereof. In certain aspects, the first IgG Fc domain polypeptide comprises a knob substitution, and the second IgG Fc domain polypeptide comprises a hole substitution. In certain aspects, a multispecific binding protein comprises a knob substitution that is selected from the group consisting of arginine (R), phenylalanine (F), tyrosine (Y), and tryptophan (W). In certain aspects, the multispecific binding protein comprises a hole substitution that is selected from the group consisting of alanine (A), asparagine (N), aspartic acid (D), glycine (G), serine(S), threonine (T), and valine (V).

### Multispecific Binding Protein Orientations

The multispecific binding proteins (e.g., bispecific binding proteins) of the disclosure may comprise a pentavalent form or an octavalent form.

In the pentavalent form, the multispecific binding protein comprises or consists of three TNFRSF ISVDs and comprises or consists of two immune checkpoint ISVDs. In the pentavalent form, the multispecific binding protein comprises or consists of three GITR ISVDs and comprises or consists of two PD-1 ISVDs.

Thus in one aspect, the disclosure provides a bispecific binding protein comprising: (a) a first binding moiety comprising at least three GITR ISVDs directly linked to each other and wherein one GITR ISVD is linked to the N-terminal end of a first IgG; (b) a second binding moiety comprising at least two PD-1 ISVDs directly linked to each other and wherein one PD-1 ISVD is linked to the N-terminal end of a second IgG; and wherein each the first and second IgG1 domains heterodimerize to form a Fc domain or variant thereof.

In some aspects, the first binding moiety consists of three GITR ISVDs.

In some aspects, the second moiety consists of two PD-1 ISVDs.

In the octavalent form, the bispecific binding protein comprises or consists of six TNFRSF ISVDs and comprises or consists of two immune checkpoint ISVDs. In the octavalent form, the bispecific binding protein comprises or consists of six GITR ISVDs and comprises or consists of two PD-1 ISVDs.

Thus in one aspect, the disclosure provides a bispecific binding protein comprising: (a) a first binding moiety comprising at least three GITR ISVDs directly linked to each other and wherein one GITR ISVD is linked to the C-terminal end of a first and a second IgG; (b) a second binding moiety comprising at least two PD-1 ISVDs wherein each PD-1 ISVD is directly linked to the N-terminal end of the first and the second IgG; and wherein each the first and second IgG1 domains form a Fc domain or variant thereof.

In some aspects, the first binding moiety consists of six GITR ISVDs.

In some aspects, the second moiety consists of two PD-1 ISVDs.

### TNFRSF Binding Moieties

In certain aspects, the multispecific binding protein of the disclosure comprises a first binding moiety that targets protein may be a TNFRSF (Tumor Necrosis Factor Receptor Super Family) member. The TNFRSF is a protein superfamily of cytokine receptors characterized by the ability to bind tumor necrosis factors (TNFs) via an extracellular cysteine-rich domain where the homology identified between family members is mainly found in the extracellular domain. With a few exceptions e.g., a nerve growth factor (NGF), all TNFs are homologous to the archetypal TNF-alpha.

In certain aspects, the multispecific binding protein of the disclosure will comprise a first binding moiety comprising at least three ISVDs that specifically bind a TNFRSF receptor (TNFRSF ISVDs) protein. In certain embodiments, the at least three TNFRSF ISVDs specifically bind the same or different TNFRSF protein(s). In certain aspects, wherein the at least three TNFRSF ISVDs specifically bind the same TNFRSF protein. In certain aspects, the at least three TNFRSF ISVDs bind to the same or different contacts on the same TNFRSF protein.

In certain aspects, the first binding moiety is an antagonist of the TNFRSF protein(s). In certain aspects, first binding moiety is an agonist of the TNSFRSF protein(s).

In certain aspects, the TNFRSF protein is selected from the group consisting of: a glucocorticoid-induced tumor necrosis factor receptor-related protein (GITR), a 4-1BB, a OX40, a tumor necrosis factor receptor 1 (TNFR1), a TNFR2, a lymphotoxin beta receptor (LTBR), a CD40, a fas receptor, a CD27, a CD30, a death receptor 3 (DR3), a DR4, a DR5, a DR6, a decoy receptor 1 (DCR1), a DCR2, a DCR3, a receptor activator of nuclear factor kappa-B (RANK), an osteoprotegerin (OPG), a tumor necrosis factor-like weak inducer of apoptosis receptor (TWEAK-R), a tachykinin precursor 1 (TACI), a B cell activating factor receptor (BAFF-R), a herpes virus entry mediator (HVEM), a nerve growth factor receptor (NGFR), a B-cell maturation antigen (BCMA), a TROY, and ectodysplasin A2 receptor (EDA2R). In certain aspects, the protein is GITR.

The term “glucocorticoid-induced TNF receptor”, hereinafter referred to as “GITR” is also known as Tumor Necrosis Receptor Superfamily 18 (TNFRSF18), Activation-Inducible TNFR Family Receptor (AITR), TEASR, CD357 and 312C2. GITR is constitutively expressed in all T cell subtypes and mostly in regulatory T cells (Treg), is up-regulated in CD4⁺/CD25 and CD8⁺/CD25⁻ effector cells following TCR stimulation and cell activation (Nocentini et al. 2007, Eur J Immunol. 37:1165-1169). GITR acts as a costimulatory molecule in effector T cell activation and regulates Treg cell suppressor activity (Esparza et al. 2005, J Immunol. 174:7869-7874).

The disclosure provides a multispecific binding protein comprising a first binding moiety comprising at least one ISVD that specifically binds GITR (GITR ISVD). In certain aspects, at least three ISVD specifically bind to GITR. In certain aspects, the GITR ISVD of the disclosure is an agonist of GITR. In certain aspects, each GITR ISVD of the multispecific binding protein is an agonist of GITR.

As used herein, an “agonist” refers to a compound that partially or fully increases, enhances, induces or stimulates one or more biological activities of a corresponding target (e.g., GITR) in vitro or in vivo. Examples of such biological activities of GITR include promoting CD4⁺ and CD8⁺ T cell survival, proliferation, NFkB signaling, interleukin-2 production and effector functions and abrogate Treg cell suppressive effects or the generation of Treg cells. As will be clear to the skilled person, such an increase in biological activity may be determined in any suitable manner and/or using any suitable (in vitro, cellular or in vivo) assay known per se, such as the assays described herein or in the prior art cited herein. In particular, the biological activity may be increased, by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more, such as 100%, compared to the biological activity in the same assay under the same conditions but without the presence of the polypeptide of the invention.

### Immune Checkpoint Proteins

As used herein, the term “immune checkpoint protein” refers to a protein expressed on the surface of immune cells (e.g., T cells) that modulates an immune response.

In certain aspects, the multispecific binding protein of the disclosure comprises comprising at least one ISVD that specifically binds to an immune checkpoint protein. In certain aspects, the second binding moiety comprises at least two ISVDs. In certain aspects, the at least two ISVDs specifically bind the same or different immune checkpoint protein(s). In certain aspects, the at least two ISVDs bind to the same or different contacts on the same immune checkpoint protein.

In certain aspects, the second binding moiety is antagonistic modulator (i.e., an inhibitor) of an immune checkpoint protein. In certain aspects the second binding moiety is an agonist (i.e., a stimulator) of the immune checkpoint protein(s).

In certain embodiments, the immune checkpoint protein is selected from a group consisting of: a programmed cell death protein 1 (PD-1), a PD ligand 1 (PD-L1), PD-L2, a cytotoxic T-lymphocyte-associated antigen 4 (CTLA-4), a T-cell immunoglobulin and mucin domain 3 (TIM-3), a lymphocyte activation gene 3 (LAG-3), a T cell immunoglobulin and immunoreceptor tyrosine-based inhibitory motif domain (TIGIT), a B and T lymphocyte attenuator (BTLA), a signaling lymphocytic activation molecule (SLAM), a leukocyte-associated immunoglobulin-like receptor 1 (LAIR1), a cluster of differentiation 244 (CD244), a CD28, a CD200 receptor 1 (CD200R1), a TREM-like transcript 2 (TLT2), a leukocyte immunoglobulin like receptor B4 (LILRB4), a killer cell immunoglobulin like receptor (KIR2DL2), and an inducible T-cell co-stimulator (ICOS). In certain aspects, the immune checkpoint protein is PD-1.

In certain aspects, the multispecific binding of the disclosure comprises at least two ISVDs specifically bind to PD-1 (PD-1 ISVDs). In certain aspects, the multispecific binding protein of the disclosure comprises at least one PD-1 ISVD which is an antagonist. In certain aspects, the PD-1 ISVD binds to an extracellular domain of a PD-1 subunit. In certain aspects, at least two PD-1 ISVDs bind to the same PD-1 subunit. In certain aspects, at least two of the PD-1 ISVDs bind to different PD-1 subunits. In certain aspects, each PD-1 ISVD is an antagonist of PD-1.

Interaction of PD-L1 with its receptor PD-1 on T cells delivers a signal that inhibits TCR-mediated activation of IL-2 production and T cell proliferation (e.g., antigen specific T cells in the lymph nodes) as well as simultaneously reducing apoptosis in regulatory T cells (e.g., anti-inflammatory, suppressive T cells). Dysregulation of the PD-1/PD-L1 interaction and related signaling has been implicated in the pathogenesis of neoplastic disorders, inflammatory disorders, and autoimmune disorders.

In certain aspects, the second binding moiety blocks the interaction of a target protein with other proteins resulting in immune modulation. For example, in certain aspects, the second binding moiety blocks the interaction of the immune checkpoint protein with other proteins resulting in immune modulation. In certain aspects, the second binding moiety blocks the interaction between PD-1 and PD-L1. PD-L1 is a membrane protein that forms a complex with its receptor PD-1. Analytical measurement to determine whether the second binding moiety blocks the interaction between PD-1 and PD-L1 are known in the art, e.g., see PD-1: PD-L1 Cell-Based Inhibitor Screening Assay Kit (BPS Biosience; catalog #79377).

In certain aspects, a multispecific binding protein blocks the PD-1/PD-L1 interaction as measured by TCR signaling in T cells expressing human PD-1 co-cultured with cells expressing PD-L1. In certain aspects, a multispecific binding protein blocks the PD-1/PD-L1 interaction as measured in Jurkat T cells expressing human PD-1 and a reporter gene co-cultured with CHO-K1 cells expressing human PD-L1 and activating TCRs in an antigen independent manner. In certain aspects, the reporter gene is a luciferase reporter gene driven by a NFAT promoter. In certain aspects, a half-maximal inhibitory concentration (IC50 [M]) of the multispecific binding protein for blocking the PD-1/PD-L1 interaction is measured by TCR signaling in T cells expressing human PD-1 co-cultured with cells expressing PD-L1 is at least 5.0×10⁻⁹, 1.0×10⁻⁹, 5.0×10⁻¹⁰, 1.0×10⁻¹⁰, 5.0×10⁻¹¹, or 1.0×10⁻¹¹ M. In certain aspects, the multispecific binding protein of the disclosure comprises a half-maximal inhibitory concentration (IC50 [M]) binding value for PD-1 is less than about 1×10⁻⁹.

In certain aspects, the multispecific binding protein of the disclosure comprises about 60% of the human PD-1 receptor is occupied when there is about 1×10⁻² nM or more of the multispecific binding protein. In certain aspects, the multispecific binding protein of the disclosure comprises about 1×10⁻¹ nM or more of the multispecific binding protein.

### Kabat, EU, and AbM Numbering

Amino acid positions in a heavy chain constant region, including amino acid positions in the CH1, hinge, CH2, CH3, and CL domains, can be numbered according to the Kabat index numbering system (see Kabat et al., in “Sequences of Proteins of Immunological Interest”, U.S. Dept. Health and Human Services, 5th edition, 1991). Alternatively, antibody amino acid positions can be numbered according to the EU index numbering system (see Kabat et al.).

In the CDR determination according to Kabat, FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.

CDR sequences can also be determined according to the AbM numbering as described in Kontermann and Dübel (2010), Antibody Engineering, vol. 2, Springer Verlag Heidelberg Berlin, Martin, Chapter 3, pp. 33-51. According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

### Specifically Binds

A polypeptide (e.g., an immunoglobulin, an antibody, an immunoglobulin single variable domain, a VHH, or generally an antigen binding molecule or a fragment thereof) that can “bind to” or “specifically bind to”, that “has affinity for” and/or that “has specificity for” a certain epitope, antigen or protein (or for at least one part, fragment or epitope thereof) is said to be “against” or “directed against” said epitope, antigen or protein or is a “binding” molecule with respect to such epitope, antigen or protein, or is said to be “anti”-epitope, “anti”-antigen or “anti”-protein (e.g., “anti”-GITR and/or PD-1).

A polypeptide that can “specifically binds” e.g., can bind to an antigen with a dissociation constant (K_{D}) of at most about 1×10⁶ M, 1×10⁻⁷ M, 1×10⁻⁸ M, 1×10⁻⁹ M, 1×10⁻¹⁰ M, 1×10⁻¹¹ M, 1×10⁻¹² M, or less, and/or to bind to an antigen with an affinity that is at least two-fold greater than its affinity for a nonspecific antigen. Specific binding of an antibody can be to a target antigen through the CDR sequences. An antibody can also specifically bind to FcRs, such as FcRn or FcγRIlla through the Fc region.

The terms “specificity”, “binding specifically” or “specific binding” refer to the number of different target molecules, such as antigens, from the same organism to which a particular binding unit, such as an ISVD, can bind with sufficiently high affinity (e.g., an ISVD that specifically binds to TNFRSF receptor protein or an immune checkpoint protein. “Specificity”, “binding specifically” or “specific binding” are used interchangeably herein with “selectivity”, “binding selectively” or “selective binding”.

Binding units, such as ISVDs, specifically bind to their designated targets. The specificity/selectivity of a binding unit can be determined based on affinity. The affinity denotes the strength or stability of a molecular interaction. The affinity is commonly given by the KD, or dissociation constant, which has units of mol/liter (or M). The affinity can also be expressed as an association constant, KA, which equals 1/KD and has units of (mol/liter)⁻¹ (or M⁻¹).

In some aspects, a multispecific binding protein of the disclosure comprises at least three TNFRSF ISVDs that specifically bind the same or different TNFRSF protein(s). In some aspects, wherein the at least three TNFRSF ISVDs specifically bind the same TNFRSF protein. In some aspects, the at least three TNFRSF ISVDs bind to the same or different contacts on the same TNFRSF protein.

In some aspects, a multispecific binding protein of the disclosure comprises at least two ISVDs that specifically bind the same or different immune checkpoint protein(s). In some aspects, the at least two ISVDs bind to the same or different contacts on the same immune checkpoint protein.

### Affinity

The affinity is a measure for the binding strength between a binding moiety (e.g., a first and second binding moiety as described herein) and a binding site on the target molecule (e.g., TNFRSF protein or an immune checkpoint protein) the lower the value of the KD, the stronger the binding strength between a target molecule and a targeting moiety. Typically, binding units used in the present technology, such as ISVDs, will bind to their targets with a dissociation constant (KD) of 10⁻⁵ to 10⁻¹² moles/liter or less, 10⁻⁷ to 10⁻¹² moles/liter or less, or 10⁻⁸ to 10⁻¹² moles/liter (i.e. with an association constant (KA) of 10⁵ to 10¹² liter/moles or more, 10⁷ to 10¹² liter/moles or more, or 10⁸ to 10¹² liter/moles). Any KD value greater than 10⁻⁴ mol/liter (or any KA value lower than 10⁴ liters/mol) is generally considered to indicate non-specific binding. The KD for biological interactions, such as the binding of immunoglobulin sequences to an antigen, which are considered specific are typically in the range of 10⁻⁵ moles/liter (10000 nM or 10 μM) to 10⁻¹² moles/liter (0.001 nM or 1 pM) or less.

Accordingly, specific/selective binding may mean that using the same measurement method, e.g., SPR-a binding unit (or polypeptide comprising the same) binds to GITR with a KD value of 10⁻⁵ to 10⁻¹² moles/liter or less and binds to related TNFRSF members with a KD value greater than 10⁻⁴ moles/liter. An example of a related TNFRSF member is 4-1BB, OX40, TNFR1 (Tumor necrosis factor receptor 1), TNFR2 (Tumor necrosis factor receptor 2), LTBR (Lymphotoxin beta receptor), CD40, Fas receptor, CD27, CD30, DR3 (Death receptor 3), DR4 (Death receptor 4), DR5 (Death receptor 5), DR6 (Death receptor 6), DCR1 (Decoy receptor 1), DCR2 (Decoy receptor 2), DCR3 (Decoy receptor 3), RANK (Receptor activator of nuclear factor kappa-B), Osteoprotegerin, TWEAK receptor, TACI, BAFF receptor, HVEM (Herpes virus entry mediator), Nerve growth factor receptor, B-cell maturation antigen, TROY and Ectodysplasin A2 receptor. Thus, in an embodiment of the present technology, the ISVD binds to (human) GITR with a KD value of 10⁻⁵ to 10⁻¹² moles/liter or less and binds to 4-1BB or OX40 of the same species with a KD value greater than 10⁻⁴ moles/liter.

Accordingly, specific/selective binding may mean that using the same measurement method, e.g., SPR-a binding unit (or polypeptide comprising the same) binds to PD-1 with a KD value of 10⁻⁵ to 10⁻¹² moles/liter or less and binds to related immune checkpoint protein members with a KD value greater than 10⁻⁴ moles/liter. An example of a related immune checkpoint protein member is cytotoxic T-lymphocyte-associated antigen 4 (CTLA-4), T-cell immunoglobulin and mucin domain 3 (TIM-3), lymphocyte activation gene 3 (LAG-3), T cell immunoglobulin and immunoreceptor tyrosine-based inhibitory motif domain (TIGIT), and B and T lymphocyte attenuator (BTLA). Thus, in an embodiment of the present technology, the ISVD binds to (human) PD-1 with a KD value of 10⁻⁵ to 10⁻¹² moles/liter or less and binds to CTLA-4, TIM-3, LAG-3, TIGIT or BTLA of the same species with a KD value greater than 10⁻⁴ moles/liter.

Specific binding to a certain target from a certain species does not exclude that the binding unit can also specifically bind to the analogous target from a different species. For example, specific binding to human GITR and/or PD-1 does not exclude that the binding unit or a polypeptide comprising the same can also specifically bind to GITR and/or PD-1 from cynomolgus monkeys (“cyno”).

In some aspects, the multispecific binding protein of the disclosure comprises least one GITR ISVD specifically binds human GITR. In some aspects, the multispecific binding protein of the disclosure comprises at least one PD-1 ISVD specifically binds human PD-1.

Specific binding of a binding unit (or a binding subunit) to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned further herein.

In some aspects, the multispecific binding protein of the disclosure comprises at least one GITR ISVD binds to an extracellular domain of a GITR subunit. In some aspects, the at least three of the GITR ISVDs bind to the same GITR subunit. In some aspects, the at least one of the GITR ISVDs can bind to different GITR subunits.

In some aspects, the multispecific binding protein of the disclosure comprises at least one PD-1 ISVD binds to an extracellular domain of a PD-1 subunit. In some aspects, at least two PD-1 ISVDs bind to the same PD-1 subunit. In some aspects, at least two of the PD-1 ISVDs bind to different PD-1 subunits.

### Cross-Reactive

An amino acid sequence is said to be “cross-reactive” for two different antigens or antigenic determinants (such as e.g., serum albumin from two different species of mammal, such as e.g., human serum albumin and cyno serum albumin, such as e.g., GITR and/or PD-1 from different species of mammal, such as e.g., human GITR and/or PD-1, cyno GITR and/or PD-1 and mouse GITR and/or PD-1) if it is specific for (as defined herein) these different antigens or antigenic determinants.

### Block or Antagonist

The terms “block”, “antagonize”, “compete”, “competing” and “-competition” are used interchangeably herein to mean the ability of an immunoglobulin, antibody, ISVD(s), VHH, multispecific or bispecific binding protein, polypeptide, or other binding agent to interfere with the binding of another protein(s), polypeptide(s), ligand(s) or binding agent(s) to a given target. In certain aspects, the blocking the given target results in inhibition or modulation of a downstream signal transduction pathway.

The extent to which an immunoglobulin, antibody, ISVD(s), VHH, multispecific or bispecific binding protein, polypeptide, or other binding agent is able to interfere with the binding of another ligand to the target, and therefore whether it can be said to “block”, can be determined using competition binding assays. Particularly suitable quantitative competitive blocking assays are described in the Examples and include e.g., a fluorescence-activated cell sorting (FACS) binding assay with GITR and/or PD-1 expressed on cells. The extent of blocking can be measured by the (reduced) channel fluorescence.

The following generally describes a suitable FACS assay for determining whether an immunoglobulin, antibody, immunoglobulin single variable domain, polypeptide or other binding agent blocks or is capable of blocking. It will be appreciated that the assay can be used with any of the immunoglobulin single variable domains and polypeptides described herein. The FACS instrument (e.g. FACS Canto; Becton Dickinson) is operated in line with the manufacturer's recommendations.

To evaluate the “blocking” or “competition” between two binding agents (such as an immunoglobulin single variable domain and a natural ligand or another binding agent) for binding GITR and PD-1, a FACS competition experiment can be performed using cells (such as e.g. Flp-In™-293 cells) overexpressing human GITR and PD-1 and the parental cells as background cell line. Different detection reagents can be used including e.g. monoclonal ANTI-FLAG® M2 antibody (Sigma-Aldrich, cat #F1804), monoclonal anti-C-myc antibody (Sigma-Aldrich, cat #WH0004609M2), monoclonal ANTI-HIS TAG antibody (Sigma-Aldrich, cat #SAB1305538), each labeled differently. A wide range of fluorophores can be used as labels in flow cytometry (such as e.g., PE (R-Phycoerythrin), 7-aminoactinomycin D (7-AAD), Acridine Orange, various forms of Alexa Fluor, Allophycocyanin (APC), AmCyan, Aminocoumarin, APC Cy5, APC Cy7, APC-H7, APC/Alexa Fluor 750, AsRed2, Azami-Green, Azurite, B ODIPY FL C5-ceramide, BCECF-AM, Bis-oxonol DiBAC2 (3), BODIPY-FL, Calcein, Calcein A M, Caroxy-H2DCFDA, Cascade Blue, Cascade Yellow, Cell Tracker Green, Cerulean, CFSE, Chromomycin A3, CM-H2DCFDA, Cy2, Cy3, Cy3.5, Cy3B, Cy5, Cy5.5, Cy7, CyPet, DAF-FM DAF-FM diacetate, DAPI, DCFH (2′7′Dichorodihydrofluorescein), DHR, Dihydrocalcein AM, Dihydrorhoadamine, Dihydrothidium, DiLC1 (5), DiOC6 (3), DiOC7 (3), dKeima-Red, DRAQ5, Dronpa-Green, various forms of DsRed dTomato, various forms of DyLight, E. coli BioParticles AF488, E2-Crimson, E2-Orange, EBFP2, ECFP, various forms of eFluor, EGFP, EGFP*, Emerald, eqFP650, eqFP670, ER-Tracker Blue-White DPX, Ethidium Bromide, Express2, EYFP, Fc OxyBurst Green, Fc OxyBurst Green 123, FITC, Fluo-3, Fluo-4, Fluorescein, Fura-2, Fura-Red, GFPuv, H2DCFDA, HcRed1, Hoechst Blue (33258), Hoechst Red (33342), Hydroxycoumarin, HyPer, Indo-1, Indo-1 Blue (Low Ca2+), Indo-1 Violet (High Ca2+), iRFP, J-Red, JC-1, JC-9, Katushka (TurboFP635), Katushka2 Kusabira-Orange, LDS 751, Lissamine Rhodamine B, various forms of Live/Dead, Lucifer yellow, Lucifer Yellow CH, Lyso Tracker Blue, Lyso Tracker Green, Lyso Tracker Red, mAmertrine, Marina Blue, mBanana, mCFP, mCherry, mCitrine, Methoxycoumarin, mHoneyDew, Midoriishi-Cyan, Mithramycin, Mito Tracker Deep Red, Mito Tracker Green, Mito Tracker Orange, Mito Tracker Red, MitoFluor Green, mKate (TagFP635), mKate2, mKeima, mKeima-Red, mKO, mKOk, mNeptune, Monochlorobimane, mOrange, mOrange2, mRaspberry, mPlum, mRFP1, mStrawberry, mTangerine, mTarquoise, mTFP1, mTFP1 (Teal), NBD, OxyBurst Green H2DCFDA, OxyBurst Green H2HFF BSA, Pacific Blue, PE (R-Phycoerythrin), PE Cy5, PE Cy5.5, PE Cy7, PE Texas Red, PE-Cy5 conjugates, PE-Cy7 conjugates, PerCP (Peridinin chlorphyll protein), PerCP Cy5.5, PhiYFP, PhiYFP-m, Propidium lodide (PI), various forms of Qdot, Red 613, RFP Tomato, Rhod-2, S65A, S65C, S65L, S65T, Singlet Oxygen Sensor Green, Sirius, SNARF, Superfolder GFP, SYTOX Blue, SYTOX Green, SYTOX Orange, T-Sapphire, TagBFP, TagCFP, TagGFP, TagRFP, TagRFP657, TagYFP, tdTomato, Texas Red, Thiazole Orange, TMRE, TMRM, Topaz, TOTO-1, TO-PRO-1, TRITC, TRITC TruRed, TurboFP602, TurboFP635, TurboGFP, TurboRFP, TurboYFP, Venus, Vybrant CycleDye Violet, Wild Type GFP, X-Rhodamin, Y66F, Y66H, Y66W, YOYO-1, YPet, ZsGreen1, ZsYellow1, Zymosan A BioParticles AF488 (see more at: http://www.thefcn.org/flow-fluorochromes). Fluorophores, or simply “fluors”, are typically attached to the antibody (e.g. the immunoglobulin single variable domain) that recognizes GITR and/or PD-1 or to the antibody that is used as detection reagent. Various conjugated antibodies are available, such as (without being limiting) for example antibodies conjugated to Alexa Fluor®, DyLight®, Rhodamine, PE, FITC, and Cy3. Each fluorophore has a characteristic peak excitation and emission wavelength. The combination of labels which can be used will depend on the wavelength of the lamp(s) or laser(s) used to excite the fluorophore and on the detectors available.

### EC50

“EC50” is the half maximal effective concentration that measures the concentration of a multispecific binding protein described herein or e.g., a first or second binding moiety of the same which induces a biological response halfway between the baseline and maximum after a specified exposure time.

To evaluate the competition between two test binding agents (termed A and B) for binding to a e.g., a first or second binding moiety target (e.g., a TNFRSF protein or an immune check point protein), a dilution series of cold (without any label) binding agent A is added to (e.g. 200 000) cells together with the labeled binding agentB*. The concentration of binding agent B* in the test mix should be high enough to readily saturate the binding sites of target (e.g., a TNFRSF protein or an immune check point protein) expressed on the cells. The concentration of binding agent B* that saturates the binding sites for that binding agent on e.g., a TNFRSF protein or an immune check point protein, expressed on the cells can be determined with a titration series of binding agent B* on cells comprising e.g., a TNFRSF protein or an immune check point protein and determination of the EC50 value for binding. To work at saturating concentration, binding agent B* can be used at 100× the EC50 concentration.

After incubation of the cells with the mixture of binding agent A and binding agent B* and cells wash, read out can be performed on a FACS. First a gate is set on the intact cells as determined from the scatter profile and the total amount of channel fluorescence is recorded. A separate solution of binding agent B* is also prepared. Binding agent B* in this solution should be in the same buffer and at the same concentration as in the test mix (with binding agent A and B*). This separate solution is also added to the cells. After incubation and cells wash, read out can be performed on a FACS. First a gate is set on the intact cells as determined from the scatter profile and the total amount of channel fluorescence is recorded. A reduction of fluorescence for the cells incubated with the mixture of binding agent A and B* compared to the fluorescence for the cells incubated with the separate solution of binding agent B* indicates that binding agent A blocks binding by binding agent B* cells expressing e.g., a TNFRSF protein or an immune check point protein.

A cross-blocking immunoglobulin, antibody, immunoglobulin single variable domain, polypeptide or other binding agent is one which will bind to e.g., a TNFRSF protein or an immune check point protein in the above competition FACS such that during the assay and in the presence of the second binding agent the recorded fluorescence is between 80% and 0.1% (e.g. 80% to 4%) of the maximum fluorescence (measured for the separate labelled immunoglobulin, antibody, immunoglobulin single variable domain, polypeptide or other binding agent), specifically between 75% and 0.1% (e.g. 75% to 4%) of the maximum fluorescence, and more specifically between 70% and 0.1% (e.g. 70% to 4%) of maximum fluorescence (as just defined above).

The competition between two test binding agents (termed A* and B*) for binding to e.g., a TNFRSF protein or an immune check point protein can also be evaluated by adding both binding agents, each labeled with a different fluorophore, to cells expressing e.g., a TNFRSF protein or an immune check point protein. After incubation and cells wash, read out can be performed on a FACS. A gate is set for each fluorophore and the total amount of channel fluorescence is recorded. Reduction and/or absence of fluorescence of one of the fluorophores indicates blocking by the binding agents for binding to cells expressing e.g., a TNFRSF protein or an immune check point protein.

Other methods for determining whether an immunoglobulin, antibody, immunoglobulin single variable domain, polypeptide or other binding agent directed against a target blocks, is capable of blocking, competitively binds or is competitive as defined herein are described e.g. in Xiao-Chi Jia et al. (Journal of Immunological Methods 288:91-98, 2004), Miller et al. (Journal of Immunological Methods 365:118-125, 2011).

In certain aspects, the multispecific binding protein of the disclosure has a half-maximal effective concentration (EC50 [M]) in a NFkB luciferase reporter assay is less than about 1×10−9. In certain aspects, the multispecific binding protein of the disclosure has an EC50 [M] in a T-cell activation assay is about 1×10−9 to about 3×10−11. In certain aspects, the multispecific binding protein of the disclosure has an EC50 [M] binding value for human GITR is less than about 1×10−10.

### Dissociation Constant

The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more than 10⁻⁴ moles/liter or 10⁻³ moles/liter (e.g. of 10⁻² moles/liter). Optionally, as will also be clear to the skilled person, the (actual or apparent) dissociation constant may be calculated on the basis of the (actual or apparent) association constant (KA), by means of the relationship [KD=1/KA]. The term “K_{D}” as used herein refers to the dissociation constant of the interaction between a particular binding protein or portion thereof and the target antigen.

### Valency

As used herein the term “valency” refers to the number of potential target binding sites in a multispecific binding protein. Each target binding site specifically binds one target molecule or specific site on a target molecule. When a polypeptide comprises more than one target binding site, each target binding site can specifically bind the same or different molecules (e.g., can bind to more than one target protein, or different epitopes on the same target protein). A subject multispecific binding protein described herein has at least one binding site (e.g., 1, 2, 3, or more) specific for TNFRSF protein as well as an immune checkpoint protein.

In some aspects, the multispecific binding protein described herein comprises at least one, two, three, four, five, six, seven, or eight ISVD(s) that specifically bind a TNFRSF receptor protein (e.g., a GITR). In some aspects, the multispecific binding protein described herein comprises at least one, two, three, four, five, six, seven, or eight ISVD(s) that specifically bind a GITR.

In some aspects, the multispecific binding protein described herein comprises at least one, two, three, four, five, six, seven, or eight ISVD(s) that specifically bind an immune checkpoint protein (e.g., a PD-1). In some aspects, the multispecific binding protein described herein comprises at least one, two, three, four, five, six, seven, or eight ISVD(s) that specifically bind a PD-1.

### About or Approximately

The term “about” or “approximately” means within about 20%, such as within about 10%, within about 5%, or within about 1% or less of a given value or range.

As used herein, “administer” or “administration” refers to the act of injecting or otherwise physically delivering a substance as it exists outside the body (e.g., a multispecific binding protein provided herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and can be continued chronically to defer or reduce the appearance or magnitude of disease-associated symptoms.

### Potency and Efficacy

As used herein, the term “potency” is a measure of the biological activity of an agent, e.g., an ISVD that is part of a multispecific binding protein described herein. Potency of a multispecific binding protein or a part of the multispecific binding protein (e.g., a first or a second binding moiety) can be determined by any suitable method known in the art, such as for instance as described in the experimental section. Cell culture-based potency assays are often the preferred format for determining biological activity since they measure the physiological response elicited by the agent and can generate results within a relatively short period of time. Various types of cell-based assays, based on the mechanism of action of the product, can be used, such as e.g., binding affinity assays to test the affinity of e.g., the first or second binding moiety to a TNFRSF protein or an immune check point protein, respectively (as further described in Example 15).

In contrast, the “efficacy” of an agent, such as an ISVD or polypeptide, measures the maximum strength of the effect itself, at saturating agent concentrations. Efficacy indicates the maximum response achievable from the agent. It refers to the ability of the agent to produce the desired (therapeutic) effect. The efficacy of an agent can be evaluated using in vitro functional assays (e.g., IL-2 secretion from T cells as described herein) or in vivo models (e.g., murine tumor modeling as described in herein).

### Effective Amount

“Effective amount” means the amount of active pharmaceutical agent (e.g., a multispecific binding protein of the present disclosure) sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

### Subject or Patient

As used herein, the terms “subject” and “patient” are used interchangeably. As used herein, a subject can be a mammal, such as a non-primate (e.g., cows, pigs, horses, cats, dogs, rats, etc.) or a primate (e.g., monkey and human). In certain aspects, the term “subject,” as used herein, refers to a vertebrate, such as a mammal. Mammals include, without limitation, humans, non-human primates, wild animals, feral animals, farm animals, sport animals, and pets.

### Therapy and Pharmaceutical Compositions

As used herein, the term “therapy” refers to any protocol, method and/or agent that can be used in the prevention, management, treatment and/or amelioration of a disease or a symptom related thereto. In some aspects, the term “therapy” refers to any protocol, method and/or agent that can be used in the modulation or depletion of a target protein from the circulation of a subject. In some aspects, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other aspects, the terms “therapies” and “therapy” refer to a biological therapy, supportive therapy, and/or other therapies useful in the modulation of an immune response to an inflammatory or autoimmune diseases in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as the administration of a multispecific binding protein provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

In certain aspects, a method of treating a neoplastic disorder comprises administering the multispecific or bispecific binding protein of the disclosure to a subject. In certain aspects, administration of the binding protein results in tumor growth inhibition by 1, 2, 3, or 4-fold relative to a reference binding protein.

In certain aspects, a method of treating an infection comprising administering the multispecific or bispecific binding protein of the disclosure to a subject. In certain aspects, a method of treating a T cell, B cell, or natural killer cell mediated disease comprising administering the multispecific or bispecific binding protein of the disclosure to a subject.

In certain aspects, a multispecific binding protein or a bispecific binding protein of the disclosure is for use in the treatment of neoplastic disorder. In certain aspects, the multispecific binding protein or the bispecific binding protein of the disclosure is for use in inhibiting tumor growth by 1, 2, 3, or 4-fold relative to a reference binding protein.

In certain aspects, a multispecific binding protein or a bispecific binding protein of the disclosure is for use in treating an infection.

In certain aspects, a multispecific binding protein or a bispecific binding protein of the disclosure is for use in treating a T cell, B cell, or natural killer cell mediated disease.

### Polynucleotides

In one aspect, polynucleotides (i.e., nucleic acid molecules) encoding a multispecific binding protein described herein or variants thereof are provided. A polynucleotide variant as used herein is about 50, 75, 80, 85, 90, 93, 95, 98, 99% or more identical to a polynucleotide that encodes a multispecific binding protein described herein.

Methods of making a multispecific binding protein comprising expressing these polynucleotides are also provided. Polynucleotides encoding a multispecific binding protein or variants thereof disclosed herein are typically inserted in an expression vector for introduction into host cells that can be used to produce the desired quantity of the claimed multispecific binding protein. Accordingly, in certain aspects, the disclosure provides expression vectors comprising polynucleotides disclosed herein and host cells comprising these vectors and polynucleotides.

### Expression Vector and Host Cell

The term “vector” or “expression vector” is used herein for the purposes of the specification and claims, to mean vectors used in accordance with the present disclosure as a vehicle for introducing into and expressing the polynucleotide sequence encoding a multispecific binding protein polypeptide in a cell. As known to those skilled in the art, such vectors can easily be selected from the group consisting of plasmids, phages, viruses and retroviruses. In general, vectors compatible with the instant disclosure will comprise a selection marker, appropriate restriction sites to facilitate cloning of the desired gene and the ability to enter and/or replicate in eukaryotic or prokaryotic cells.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

One or more genes encoding a multispecific binding protein can also be expressed non-mammalian cells such as bacteria, yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides can become part of inclusion bodies. The polypeptides can be isolated, purified, and then assembled into functional molecules.

In addition to prokaryotes, eukaryotic cells can also be used. Saccharomyces cerevisiae, or common baker's yeast, is the most commonly used, although a number of other strains are commonly available.

In certain aspects, the expression vector comprises a nucleic acid encoding the multispecific binding protein of the disclosure. In certain aspects, a host or host cell comprises a nucleic acid encoding the multispecific binding protein of the disclosure.

### EXAMPLES

### 5.10 Example 1: Generation of Anti-PD-1 ISVD Molecule

### 5.10.1 Immunizations

To induce a heavy-chain antibody (HcAb)-dependent humoral immune response, three llama and three alpaca were immunized with recombinant human PD-1 (ECD extracellular domain hPD-1::Halotag-histag) (in house produced) or with full length huPD1 cDNA. The camelids initially immunized with cDNA were further boosted with the recombinant PD-1 extracellular domain. Protein-immunized camelids on the other hand, were subjected to the full immunization schedule using PD-1 cDNA.

### 5.10.2 Library Constructions and Phage Display Selections

Immune blood samples were taken at regular intervals, serum responses were determined, and total RNA was prepared from the isolated PBL. From these blood samples, peripheral blood mononuclear cells (PBMCs) were prepared using Ficoll-Hypaque according to the manufacturer's instructions (Amersham Biosciences, Piscataway, NJ, US). From the PBMCs, total RNA was extracted and used as starting material for RT-PCR to amplify the VHH-encoding DNA segments, essentially as described in WO 2005/044858. Subsequently, phages were prepared according to standard protocols (see for example the prior art and applications filed by Ablynx N.V. cited herein) and stored after filter sterilization at 4° C. for further use.

For general phage display selection techniques, reference is made to Antibody Phage Display: Methods and Protocols (First Edition, 2002, O'Brian and Aitken eds., Humana Press, Totowa, NJ). Two rounds of panning phage display selections were performed using the llama and alpaca VHH immune libraries in alternating selection rounds on human, and cynomolgus PD-1 expressing cell lines (in house made preB-cells) or recombinant proteins. The output from the selection were plated onto LB/amp/2% glu plates. Colonies were picked and grown in 96 deep well plates (1 ml volume) and induced by adding IPTG for ISVD expression. Periplasmic extracts (volume: ~80 μl) were prepared according to standard methods (see for example the prior art and applications filed by applicant cited herein).

### 5.10.3 Primary Screening and Sequencing

Primary screening of 4116 periplasmic extracts (PE) was performed by FACS binding on CHO cell lines expressing human or cyno PD-1 (in house made) and by FACS competition assay using hPD-L1-Fc Biotinylated (Sino Biological) as competitor to CHO cells expressing human or cyno PD-1.

In short, the binding of PE containing anti-PD-1 ISVDs to human and cyno PD-1 overexpressed on CHO Flp-In cell lines was evaluated in flow cytometry in a fluorescent cell-barcoding setup. A final dilution of 1/10 of the PE was applied to the cells and competed against a fixed concentration human PD-L1-Fc Biotinylated equal to 10 nM. ISVDs and human PD-L1-Fc were allowed to associate for 3 h in the dark on a shaker at 4° C. in FACS buffer (PBS supplemented with 10% FBS and 0.05% azide). Cells were washed twice by centrifugation and probed with Brilliant Violet 421™ anti-DYKDDDDK Tag anti-FLAG antibody (Biolegend; cat #: 637321), to detect bound ISVD to PD-1 and with Streptavidin-Phycoerythrin (BD-Pharmingen cat #554061) to detect bound human PD-L1-Fc to PD-1, for 30 minutes at 4° C. Cells were washed and then analysed via an iQue Screener (Sartorius). The multiplexed cell lines were separated via their respective fluorescent barcodes using the proper gating strategy.

In addition, all peri extracts generated in this campaign were screened in dose dependent NFAT PD-1 reporter assay (PD-1/PD-L1 blocking assay) which was correlated with competition FACS results. The assay (Promega J1250 and J1255) consists of two genetically engineered cell lines: Jurkat T cells expressing human PD-1 and a luciferase reporter gene driven by an NFAT promotor are used as PD-1 Effector Cells and CHO-K1 cells expressing human PD-L1 and an engineered cell surface protein which are designed to activate cognate TCRs in an antigen-independent manner are used as target cells. When the two cell types are co-cultured, the PD-1/PD-L1 interaction inhibits TCR signaling and NFAT-RE-mediated luminescence. In brief, the CHO-K1 cells expressing human PD-L1 cells were thawed in recovery medium (90% Ham's F12 (Gibco, 31765)+10% HI FCS (Gibco, 10082147)) and 80 L (10000 cells per well) were transferred to a 384-well plate (Greiner bio one, 781080) and incubated overnight in a 5% CO2 atmosphere at 37° C. The next day, the Jurkat T cells expressing human PD-1 were thawed in recovery medium, washed and transferred to assay medium (RPMI (Gibco 1640)+1% HI FBS) to a concentration of 7.5×105 cells per mL. Meanwhile, the 384-well plate containing the target cells was taken out of the incubator, and all the media was removed. For analysis of concentration-dependent T-cell activation, serial dilutions of anti-PD1 ISVD proteins were added to the cells and incubated for 10 min at room temperature. Next, PD-1 Jurkat NFAT effector cells (final conc. 15,000 cells per well) were added, and the plate was incubated for 6 h in a 5% CO2 atmosphere at 37° C. After the incubation, the Bio-Glo™ Luciferase was thawed in assay medium in a refrigerator overnight or in a room temperature water bath on the day of assay. The Bio-Glo™ Luciferase Assay Buffer was equilibrated to ambient temperature, protected from light. All the Bio-Glo™ Luciferase assay medium was transferred into the amber bottle containing the Bio-Glo™ Luciferase assay substrate and mixed by inversion until the substrate was thoroughly dissolved. The reconstituted Bio-Glo™ Reagent was equilibrated and stored at ambient temperature (22-25° C.) protected from light before adding to assay plates. After incubation of the assay plates, these plates were equilibrated to ambient temperature for 10 to 15 minutes. Next 40 μL of Bio-Glo™ Reagent was added to each well. After the incubation at ambient temperature for 5 to 30 minutes in the dark, the luminescence was measured using the BMG

### PheraStar or Clariostar.

Human-cyno cross-reactive PD-1 blockers (N=245) and non-blockers (N=230) were sequenced which resulted in 209 unique clones. 53 were new members of old families and 156 were new clones clustered into 52 novel families. All unique clones (both antagonist and non-antagonists) were further evaluated in SPR-based off-rate screen.

### 5.11 Example 2: Initial Characterization of the Unique Sequences

### 5.11.1 Off-Rate Analysis of the Unique Clones

All unique clones from the sequence analysis were subjected to off-rate analysis towards human and cyno PD-1 by Surface Plasmon Resonance (SPR) on a ProteOn XPR36 (Bio-Rad Laboratories), SPR-32 Bruker and an Cytiva Biacore 8K+ instrument. The experiment was performed at 25° C. Thereto, recombinant hPD-1::Halotag-histag and cPD1::Halotag-histag (In house produced) were immobilized on GLC sensorchip (Bio-Rad Laboratories), or a High-Capacity Amine chip (Bruker) or on a CM5 chip (GE Healthcare Life Sciences) via amine coupling, using EDC and NHS chemistry (running buffer used: HBS-EP+, pH7.4). Periplasmic extracts were diluted 1:10 in running buffer (HBS-EP+, pH7.4) and they were injected over the sensor surfaces (association: 120 s, dissociation: 900 s, 45 μL/min). Between sample injections, the surfaces were regenerated with 100 mM HCL (Merck Millipore). Several buffer blanks were injected for double referencing. Data was double referenced by subtracting a reference ligand lane and a blank buffer injection. Processed sensorgrams were analysed based on the 1:1 interaction model (Langmuir binding model) using ProteOn Manager 3.1.0 software (Bio-Rad Laboratories) or using SPR-32 Analyzer 3.2 analysis software or using the Biacore Insight Evaluation software.

Kinetic screen confirmed overall good human-cyno cross-reactivity of the clones and revealed that majority of the ISVDs had kd values in the range of 1E-04 or even 1E-05 1/sec. Based on all screening data, a Lead Panel was selected.

### 5.11.2 Selection of Initial Lead Panel

Based on binding and competition in FACS, on NFAT results, on sequence analysis, and on off-rate, 10 clones (Table B-1) were selected for further characterization, expression, purification and biophysical characterization.

TABLE B-1
Results of the screening of periplasmic extracts in binding FACS, competition FACS, NFAT PD-1 reporter assay and off rate

| | | bFACS CHO Flp-In | | cFACS CHO Flp-In | | NFAT | | SPR/ off rate | |
|---|---|---|---|---|---|---|---|---|---|
| | | cyno PD-1 | human PD-1 | cyno PD-1 | human PD-1 | % Inhibition | IC50 | koff (1/s) | koff (1/s) |
| Original ID | Family | Ratio* | Ratio* | % Inhibition | % Inhibition | max | rel nM | huPD1 | cyPD1 |
| A0294PMP047E02 | 68 | 484 | 615 | 101 | 101 | 76 | 1.2 | 2.90E−04 | 3.80E−04 |
| A0294PMPPAR1 | 68 | 623 | 712 | 100 | 100 | 57 | 1.4 | 4.40E−04 | 6.00E−04 |
| A0294PMPPAR2 | 7 | 539 | 642 | 100 | 99 | 40 | 3.4 | 3.90E−04 | 4.30E−04 |
| A0294PMP037F03 | 111 | 209 | 148 | 100 | 100 | 75 | 0.9 | ND | ND |
| A0294PMP041A11 | 76 | 134 | 139 | 100 | 100 | 63 | 1.5 | 4.10E−04 | 5.30E−04 |
| A0294PMPPAR3 | 107 | 410 | 753 | 100 | 101 | 63 | 2.4 | 1.00E−04 | 4.10E−04 |
| A0294PMP040C12 | 105 | 460 | 493 | 100 | 100 | 17 | 0.5 | 1.10E−04 | 1.60E−04 |
| A0294PMP0PAR4 | 11 | 118 | 123 | 100 | 100 | 46 | 1.5 | 3.60E−04 | 4.30E−04 |
| A0294PMP0PAR5 | 102 | 448 | 473 | 100 | 100 | 27 | 2.7 | 1.20E−04 | 1.00E−04 |
| A0294PMP038E01 | 76 | 221 | 215 | 100 | 100 | 67 | 6.0 | ND | ND |

Ratio*: ratio of VHH samples over the in-plate negative control

### 5.12 Example 3: Generation of ISVD Protein Expression Constructs

For the selected clones, ISVD protein encoding DNA fragments, obtained by PCR with specific combinations of forward FR1 and reverse FR4 primers each carrying a unique restriction site, were digested with the appropriate restriction enzymes and ligated into the matching cloning cassettes of ISVD protein expression vectors (described below). The ligation mixtures were then transformed to electrocompetent Escherichia coli TG1 (60502, Lucigen, Middleton, WI) or TOP10 (C404052, ThermoFisher Scientific, Waltham, MA) cells which were then grown under the appropriate antibiotic selection pressure (kanamycin or Zeocin). Resistant clones were verified by Sanger sequencing of plasmid DNA (LGC Genomics, Berlin, Germany).

Monovalent ISVD proteins were expressed in E. coli TG1 from a plasmid expression vector containing the lac promoter, a resistance gene for kanamycin, an E. coli replication origin and an ISVD protein cloning site preceded by the coding sequence for the OmpA signal peptide. In frame with the ISVD protein coding sequence, the vector codes for a C-terminal FLAG3 and HIS6 tag (SEQ ID NO: 4782). The signal peptide directs the expressed ISVD protein to the periplasmic compartment of the bacterial host.

Multivalent ISVD proteins were expressed in P. pastoris. The yeast expression vectors contain the AOX1 promoter and terminator, a resistance gene for Zeocin and the coding information for the Saccharomyces cerevisiae α-mating factor signal peptide. The ISVD protein building blocks were combined with GS linkers and cloned in the expression vector via Golden Gate cloning (Engler C, Marillonnet S. Golden Gate cloning. Methods Mol Biol. 2014; 1116:119-31). The expression vectors contain two Bpil restriction sites for cloning the PCR-amplified monovalent ISVD proteins together with the GS linkers included in one or multiple vectors. All these elements are flanked by Bpil sites. The use of unique nucleotide overhangs for each position of the cloning cassette allows seamless ligation in a pre-defined order. After Sanger sequence confirmation, plasmid DNA derived from E. coli TOP10 was transformed by electroporation into in-house prepared hypercompetent P. pastoris, strain NRRL Y-11430 (ATCC 76273).

HIS6-tagged (SEQ ID NO: 4782) ISVD protein were purified by immobilized metal affinity chromatography (IMAC) followed by a desalting step and if necessary, gel filtration chromatography (Superdex column, GE Healthcare) in PBS. Else, ISVD protein were purified on Protein A followed by a desalting step and if necessary, gel filtration chromatography (Superdex column, GE Healthcare) in PBS. The purity and integrity of the ISVD protein was verified by SDS-PAGE and mass spectrometry.

### 5.13 Example 4: Affinity Determination

Binding affinities of the purified ISVD protein were evaluated by means of an SPR based assay on a ProteOn XPR36 (Bio-Rad Laboratories), or a SPR-32 Bruker or an Cytiva Biacore 8K+ instrument. Recombinant hPD-1::Halotag-histag and cPD-1::Halotag-histag (in house produced) were immobilized on a GLC sensorchip (Bio-Rad Laboratories), or a High-Capacity Amine chip (Bruker) or on a CM5 chip (GE Healthcare Life Sciences) via amine coupling, using EDC and NHS chemistry (running buffer used: HBS-EP+, pH7.4). Purified ISVDs were injected for 2 minutes (flow rate 45 μL/min) at different concentrations (range depending on the ISVD) and dissociation was followed for 1200 s. Data was double referenced by subtracting a reference ligand lane and a blank buffer injection (1×HBS-EP+, pH7.4 at 25° C.). Evaluation of the association/dissociation data was performed by fitting a 1:1 interaction model (Langmuir binding model) using ProteOn Manager 3.1.0 software (Bio-Rad Laboratories) or using SPR-32 Analyzer 3.2 analysis software or using the Biacore Insight Evaluation software.

Binding affinities of the purified ISVDs for cyno PD-1 and human PD-1 evaluated by means of an SPR are given in Table B-2a.

TABLE B-2a
Affinity determination of monovalent ISVD proteins to human and cynomolgus PD-1

| | Cyno PD-1 | | | Hu PD-1 | | |
|---|---|---|---|---|---|---|
| Original ID | kon (1/Ms) | koff (1/s) | KD (M) | kon (1/Ms) | koff (1/s) | KD (M) |
| A0294047E02 | 2.8E+05 | 8.2E−06 | 2.9E−11 | 3.0E+05 | 1.3E−05 | 4.3E−11 |
| A02940PAR1 | 3.0E+05 | 9.3E−06 | 3.1E−11 | 3.5E+05 | 2.1E−05 | 6.0E−11 |
| A02940PAR2 | 5.7E+05 | 7.1E−05 | 1.3E−10 | 5.4E+05 | 1.6E−04 | 2.9E−10 |
| A0294037F03 | 4.9E+04 | 1.5E−04 | 3.1E−09* | 8.8E+04 | 6.6E−05 | 7.5E−10* |
| A0294041A11 | 4.1E+05 | 1.1E−04 | 2.6E−10 | 3.7E+05 | 1.5E−04 | 4.0E−10 |
| A02940PAR3 | 1.6E+05 | 3.5E−04 | 2.2E−09 | 2.5E+05 | 5.6E−05 | 2.2E−10 |
| A0294040C12 | 3.4E+05 | 2.4E−05 | 7.1E−11 | 3.0E+05 | 3.5E−05 | 1.2E−10 |
| A02940PAR4 | 3.6E+05 | 2.4E−04 | 6.6E−10 | 3.3E+05 | 3.4E−04 | 1.0E−09 |
| A02940PAR5 | 3.1E+05 | 1.1E−04 | 3.7E−10* | 3.1E+05 | 1.4E−04 | 4.5E−10* |
| A02940PAR6 | 1.3E+05 | 1.9E−04 | 1.5E−09* | 1.3E+05 | 2.5E−04 | 1.9E−09* |

*indicative parameters due to heterogeneous profile

### 5.14 Example 5: Biophysical Characterization of the ISVD Proteins

### 5.14.1 Thermal Shift Assay

The thermal shift assay (TSA) was performed in a 96-well plate on a qPCR machine (LightCycler 480II, Roche). Per row, one ISVD protein was analysed in the following pH range: 4, 5, 6, 7, 8 and 9. In some cases, six different formulation buffers were also included, namely 20 mM acetate (pH 5), 20 mM histidine (pH 6) or 20 mM phosphate (pH 7), either with or without 8% sucrose.

Per well, 5 μL of ISVD protein sample (0.8 mg/mL in D-PBS) was added to 5 μL of Sypro Orange (40× in MilliQ water; Invitrogen, Cat. No. S6551) and 10 μL of buffer (100 mM phosphate, 100 mM borate, 100 mM citrate and 115 mM NaCl with a pH ranging from 4 to 9). A temperature gradient (37 to 99° C. at a rate of 0.03° C./s) was applied, which induced unfolding of the ISVD proteins, and hence exposure of hydrophobic patches. Binding of Sypro Orange to those hydrophobic patches, caused increase in fluorescence intensity, which was measured (Ex/Em=465/580 nm). The inflection point of the first derivative of the fluorescence intensity curve at pH 7 served as a measure of the melting temperature (Tm).

The Tm values obtained for the selected ISVD clones are shown in Table B-2b.

### 5.14.2 Analytical Size Exclusion Chromatography

Aggregation and oligomerization propensity of the ISVD proteins was investigated by analytical size exclusion chromatography (SE-HPLC). To this end, 8 μg of ISVD protein sample (0.5 mg/ml in D-PBS) and 10 μL of the BioRad standard (Cat. No. 151-1901, 1/10 diluted in D-PBS) were analysed on a Waters Xbridge column (particle size: 3.5 μm, pore size 200 Å, ID 7.8 mm). An Arginine buffer [10 mM Phosphate+150 mM Arginine+10% 1-propanol+0.02% NaN3 (pH 7.0)] was used as mobile phase with a flowrate of 0.5 ml/minute. The overall SE-HPLC behaviour was determined based on the following three parameters: peak area of the main peak (>90%=pass), percentage recovery (>80%=pass) and retention time versus the 1.35 kDa BioRad standard peak.

The results of the SE-HPLC for the selected ISVD clones are shown in Table B-2b.

### 5.14.3 Oligomerization Assay

Oligomerization propensity of monovalent ISVD proteins under stressed conditions (1 week at 45° C.) was investigated by analytical size exclusion chromatography (SE-HPLC). For this, ISVD proteins with a 3×FLAG-His6 tag (SEQ ID NO: 4782), produced in E. coli, purified via IMAC followed by preparative SEC, filtered (0.22 μm), at a concentration of 1 mg/ml (D PBS) were used. The SE-HPLC profiles of two 100 μL aliquots were compared: one sample was incubated for 1 week at −20° C. and the other sample for 1 week at 45° C. Samples were cleared by centrifugation for 5 minutes at 20000 RCF and subsequently analysed on a Waters Xbridge column (mobile phase 10 mM Phosphate+150 mM Arginine+10% 1-propanol pH 7, flow rate 0.5 mL/minute). The difference in relative pre-peak areas of stressed (+45° C.) and non-stressed samples (−20° C.) was calculated and reported as 4% oligo (=% oligo 1W 45° C.-% oligo T0).

The Δ% oligo values obtained for the selected ISVD clones are shown in Table B-2b.

### 5.14.4 Onset of Aggregation Temperature Determination (Tagg Assay)

The temperature at which a ISVD protein starts to aggregate (=temperature of onset of aggregation =Tagg) was determined by Dynamic Light Scattering (DLS) using the DynaPro Plate reader (Wyatt). For this, ISVD proteins with a 3×FLAG-His6 tag (SEQ ID NO: 4782), produced in E. coli, purified via IMAC followed by preparative SEC, filtered (0.22 μm), at a concentration of 1 mg/ml (D-PBS) were used. After thawing the sample was filtered over a 0.1 μm membrane and centrifuged for 5 min at 14000 rpm. Samples of 30 μL (4 replicates) were heated from 40 to 80° C. at a constant rate of 0.25° C./minute with continuous recording of the light scattering intensities. The Hydrodynamic radius derived from the measured intensities was plotted against the temperature to determine the temperature at which the radius started to increase (=Tagg, ° C.).

The Tagg (° C.) values obtained for the selected ISVD clones are shown in Table B-2b.

TABLE B-2b
Biophysical characterization of monovalent ISVD proteins

| | Tm | | | Δ % |
|---|---|---|---|---|
| Original ID | pH 7 | sSEC | Tagg | Olig |
| A0294047E02 | 75.9 | no liabilities | 56.00 | 0.0 |
| A02940PAR1 | 72 | no liabilities | ND | ND |
| A02940PAR2 | 70.1 | no liabilities | 55.00 | 0.0 |
| A0294037F03 | 64.2 | uncleaved signal peptide | 59.00 | 0.0 |
| A0294041A11 | 65.2 | no liabilities | 70.00 | 0.0 |
| A02940PAR3 | 77.8 | (possible) | Data | 1.3 |
| | | oligomerisation 1-5% | inconclusive | |
| A0294040C12 | 68.6 | no liabilities | 74.00 | 0.1 |
| A02940PAR4 | 67.1 | no liabilities | ND | ND |
| A02940PAR5 | 65.7 | no liabilities | ND | ND |
| A02940PAR6 | 66.6 | no liabilities | ND | ND |

### 5.15 Example 6: Characterization in Cell-Based Assay

The monovalent lead panel ISVD proteins were characterized in binding FACS, competition FACS, and in NFAT PD-1 reporter assay (PD-1/PD-L1 blocking assay).

### 5.15.1 Binding FACS and Competition FACS Using hPD-L1-Fc as Competitor on CHO Cells Expressing Human or Cyno PD-1

Binding of purified anti-PD1 ISVD protein to human and cyno PD-1 overexpressed on CHO Flp-In cell lines was evaluated in flow cytometry. A serial dilution of ISVDs starting at 200 nM was applied to the cells. ISVDs were allowed to associate for 30 minutes at 4° C. in FACS buffer (PBS supplemented with 10% FBS and 0.05% azide). Cells were washed by centrifugation and probed with a mouse anti-FLAG antibody (Sigma; cat #: F1804) or ABH0074 (AB00003239, in-house developed antibody using hybridoma technology in mice) (10 μg/mL diluted in assay buffer) for 30 minutes at 4° C., to detect ISVD bound to PD-1. Cells were washed by centrifugation and subsequently probed with R-Phycoerythrin-conjugated AffiniPure F(ab′)₂ Fragment Goat Anti-mouse IgG, Fc γ Fragment Specific (Jackson ImmunoResearc, 115-116-071) a phycoerythrin-labelled goat anti-mouse IgG antibody to detect the mouse anti-FLAG antibody or with allophycocyanin-conjugated AffiniPure Goat Anti-Mouse IgG (subclasses 1+2a+2b+3), Fcγ Fragment Specific (Jackson ImmunoResearch, 115-135-164), respectively. Plates were finally washed 3 times and cells were resuspended in PI (Sigma-Aldrich, P4170, 1000-fold diluted in assay buffer) or DAPI solution (BD Biosciences, 564907, 5000-fold diluted in assay buffer) or TO-PRO® lodide (Life Technologies, T3605). Cell suspensions were analyzed with iQue Screener PLUS 3 (Intellicyt). For characterization, EC50 s were estimated by dose response modelling. Curves were fit using 4 parameter logistic regression in GraphPad (GraphPad Software Inc.).

Competition of purified anti-PD-1 ISVDs with human hPD-L1-Fc Biotinylated (Sino Biological) for human or cyno PD1 overexpressed on CHO Flp-In cell lines was evaluated in flow cytometry. A serial dilution of ISVDs starting at 1 μM was applied to the cells and competed against a fixed concentration human PD-L1-Fc equal to 20 nM. ISVDs and human PD-L1-Fc were allowed to associate for 3 hours on a shaker at 4° C. in FACS buffer (PBS supplemented with 10% FBS and 0.05% azide). Cells were washed twice by centrifugation and probed with R-Phycoerythrin-conjugated AffiniPure F(ab)2 Fragment Goat Anti-Human IgG Fcγ Fragment Specific (Jackson ImmunoResearch; cat #: 109-116-170) for 30 minutes at 4° C., to detect bound human PD-L1-Fc to PD-1. Cells were washed and incubated with 5 nM of TOPRO-3 to stain for dead cells which were subsequently removed during the gating procedure. The cells were then analysed via iQue Screener PLUS 3 (Intellicyt).

Results of the binding to human and cyno PD-1 as well as competition with human PD-L1-Fc in competition FACS are shown in Table B-3.

TABLE B-3
Results of binding and competition FACS of purified ISVDs on
CHO cells with hPD-L1 Fc as competitor

| | Binding FACS | | Competition FACS | |
|---|---|---|---|---|
| | (EC50) (M) | | (IC50) (M) hPD-L1 Fc | |
| Original ID | CHO-huPD-1 | CHO-cyPD-1 | CHO-huPD-1 | CHO-cyPD-1 |
| A0294047E02 | 1.04E−08 | 2.71E−08 | 5.11E−09 | 5.76E−09 |
| A02940PAR1 | 1.36E−08 | 9.71E−09 | 7.36E−09 | 7.94E−09 |
| A02940PAR2 | 1.84E−08 | 1.33E−08 | 1.76E−09 | 2.92E−09 |
| A0294037F03 | 8.33E−09 | 9.02E−09 | 5.94E−09 | 4.70E−09 |
| A0294041A11 | 1.23E−08 | 6.43E−09 | 6.07E−09 | 1.19E−08 |
| A02940PAR3 | 1.86E−08 | 1.42E−08 | 7.55E−09 | 8.57E−09 |
| A0294040C12 | 4.3E−08 | 1.24E−08 | 3.08E−09 | 3.57E−09 |
| A02940PAR4 | 1.38E−09 | 2.01E−09 | 5.05E−09 | 7.14E−09 |
| A02940PAR5 | 1.22E−08 | 1.17E−08 | 2.97E−09 | 3.21E−09 |
| A02940PAR6 | 8.90E−09 | 9.56E−09 | 5.11E−09 | 5.76E−09 |

### 5.15.2 PD-1/PD-L1 Blocking Assay

All purified leads were tested on a functional NFAT reporter assay, as described above (see 6.1.3). A serial dilution of purified ISVD proteins starting at 500 nM or 1 μM was applied to the CHO-K1 cells expressing human PD-L1 and incubated for 10 min at room temperature. Next, PD-1 Jurkat NFAT effector cells (final conc. 15,000 cells per well) were added, and the plate was incubated for 24 h in a 5% CO2 atmosphere at 37° C. After incubation of the assay plates, these plates were equilibrated to ambient temperature for 10 to 15 minutes. Next 40 μL of equilibrated Bio-Glo™ Reagent was added to each well. After the incubation at ambient temperature for 5 minutes in the dark, the luminescence was measured using the BMG PheraStar or Clariostar.

The Bio-Glo™ Luciferase was thawed in assay medium in a refrigerator overnight or in a room temperature water bath on the day of assay. The Bio-Glo™ Luciferase Assay Buffer was equilibrated to ambient temperature, protected from light.

Results of the PD-1/PD-L1 blocking assay are shown in Table B-4.

TABLE B-4
Results of PD-1/PD-L1 blocking assay with purified ISVD molecules

| Original ID | IC50 (nM) |
|---|---|
| A0294047E02 | 1.56 |
| A02940PAR1 | 1.73 |
| A02940PAR2 | 4.23 |
| A0294037F03 | 4.62 |
| A0294041A11 | 5.82 |
| A02940PAR3 | 6.08 |
| A0294040C12 | 7.75 |
| A02940PAR4 | 11.4 |
| A02940PAR5 | 27.0 |
| A02940PAR6 | 27.2 |
| Benchmark 1 | 0.8 |
| Benchmark 2 | 2.48 |

### 5.15.3 Conclusion

The functional and biophysical assays allowed a clear ranking of the initial lead panel. Monovalent clones from different families that had potency in the PD-1/PD-L1 blocking assay in the same range as the bivalent benchmarks, that had good potency in FACS, and good affinities for cyno and human PD-1, as well as good biophysical characteristics, were selected. This led to a filtered lead panel of 6 ISVDs.

### 5.16 Example 7: Functional Testing of Monovalent ISVDs in Tetanus-Toxoid Antigen-Specific Co-Culture Assay (TT Assay)

The anti-PD-1 ISVD proteins were tested for primary T cell activation (monitored via IFNγ production) in an autologous Tetanus Toxoid recall assay. In short, PBMC cells isolated from healthy donors were retrieved from cryogenic storage and thawed in culture medium (RPMI 1640 Medium, GlutaMAX™ Supplement, HEPES (Life Technologies-Gibco, 72400-021) supplemented with 10% heat-inactivated FBS (Sigma, F7254) and 1× penicillin/streptomycin (Life Technologies, 15140)). Monocytes were isolated by negative magnetic separation according to the instructions of the supplier of the human monocyte isolation kit (REA00001940, Stemcell, 19359) and cultured for 7 days in culture medium supplemented with 500 IU/ml of IL4 (PRT00001504, R&D Systems, 204-IL/CF) and 800 IU/mL of GM-CSF (PRT00000011, R&D Systems, 215-GM/CF). After 7 days, the monocytes had differentiated to immature dendritic cells (iDC). The iDCs were harvested and frozen in liquid nitrogen for later use. From the same donors, PBMC were thawed in assay medium (RPMI 1640 Medium, GlutaMAX™ Supplement, HEPES, 25 mM Hepes), supplemented with 10% heat-inactivated human AB serum (BioIVT, SM-612-Hsi) and 1× penicillin/streptomycin and cultured for 7 days in assay medium supplemented with 0.5 μg/mL Tetanus Toxoid (TT) (PRT00000467, Calbiochem, 582231). After 7 days, the TT-specific T cells were enriched. The cells were harvested and frozen in liquid nitrogen for later use. For the anti-PD-1 ISVD proteins characterization assay, iDCs were thawed in assay medium, seeded in 96-well round-bottom plates (Greiner, 650180) at 5000 cells/well in assay medium supplemented with 0.5 μg/ml TT and incubated for 4 hours in a 5% CO2 atmosphere at 37° C. Autologous TT-enriched T cells were thawed in assay medium and 100,000 cells/well were added to the iDC and a dilution series of the anti-PD-1 ISVD proteins was added. After 3 days of co-culture, cell supernatants were harvested and IFNγ concentration was determined via ELISA.

TABLE B-5
EC50 values of purified (monovalent) ISVDs in the TT assay

| | EC50 (M) for IFN-gamma production | |
|---|---|---|
| Original ID | Donor 1 | Donor 2 |
| A0294047E02 | 2.20E−09 | 2.80E−09 |
| A02940PAR2 | 3.89E−08 | 1.65E−08 |
| A0294037F03 | 2.69E−08 | 3.37E−08 |
| A0294041A11 | 4.32E−08 | 3.24E−08 |
| A02940PAR3 | 1.25E−08 | 3.55E−08 |
| A0294040C12 | 5.68E−08 | 3.60E−08 |

Based on TT assay and on all other characterization data, A0294047E02, A0294037F03, A0294041A11 and A0294040C12 were selected for sequence optimization and formatting. The sequences of the 4 ISVD molecule leads are depicted in Table B-6.

TABLE B-6
Sequence of (monovalent) ISVD lead molecules

| Original ID | Sequence |
|---|---|
| A0294047E02 | EVQLVESGGGLVQSGGSLRLSCAATGFTLDYYVIGWFRQAPGKEREGVACLSSNGDRINYADSVKGR |
| | FTISRDIAKNTVYLQMNSLKPEDTAVYHCAAGTSTTVRDMCGIMYLYDYWAQGTLVTVSS (SEQ ID |
| | NO: 402) |
| A0294037F03 | EVQLVESGGGLVQTGGSLRLSCAASGRMHSINAMAWFRQVPGKEREFVALISWGNGITYYEDSMK |
| | GRFTISRDNAKNTMYLQMNSLKPEDTARYFCAAESYGSSWYDSWGQGTLVTVSS (SEQ ID NO: |
| | 3395) |
| A0294041A11 | EVQLVESGGGLVQLGGSLRLSCAAPGDTFSENDIGWFRQTPGKEREFVAAIVWPGGETYYRDSVKGR |
| | FTLSRDNAKNTVYLQMNNLKPEDTANYSCAVKIFSKTGTYSASWEYDFWGQGTLVTVSS (SEQ ID |
| | NO: 793) |
| A0294040C12 | EVQLVESGGGLVQPGGSLRLSCTPSGFTLYDHAVGWFRQAPGKEREGVSVISSSGAHEWYVDSVKG |
| | RFTIFRDISKNTIYLQMNSLKPEDTAVYYCAAGGMWYSDDGSEYWGQGTLVTVSS (SEQ ID NO: |
| | 4056) |

### 5.17 Example 8: Formatting of ISVD Molecule Leads

### 5.17.1 Preparation of Formatted ISVDs

The four ISVD molecule leads were formatted to multivalent constructs with and without anti-albumin binding ISVD (Table B-7).

TABLE B-7
Formatted anti-hPD-1 ISVD constructs

| Construct | BB1 | Linker | BB2 | Linker | BB3 | Tag |
|---|---|---|---|---|---|---|
| A029400058 | A0294047E02 | 9GS | A0294047E02 | 9GS | ALB23002 | FLAG3-HIS6 |
| | | | | | | (SEQ ID NO: |
| | | | | | | 4782) |
| A029400063 | ALB23002 | 9GS | A0294047E02 | 9GS | A0294047E02 | FLAG3- |
| | | | | | | HIS6(SEQ ID |
| | | | | | | NO: 4782) |
| A029400064 | A0294047E02 | 9GS | ALB23002 | 9GS | A0294047E02 | FLAG3- |
| | | | | | | HIS6(SEQ ID |
| | | | | | | NO: 4782) |
| A029400062 | A0294047E02 | 9GS | A0294047E02 | | | FLAG3- |
| | | | | | | HIS6(SEQ ID |
| | | | | | | NO: 4782) |
| A029400065 | A0294037F03 | 9GS | A0294037F03 | 9GS | ALB23002 | FLAG3- |
| | | | | | | HIS6(SEQ ID |
| | | | | | | NO: 4782) |
| A029400061 | A0294037F03 | 9GS | A0294037F03 | | | FLAG3-HIS6 |
| | | | | | | (SEQ ID NO: |
| | | | | | | 4782) |
| A029400066 | A0294041A11 | 9GS | A0294041A11 | 9GS | ALB23002 | FLAG3-HIS6 |
| | | | | | | (SEQ ID NO: |
| | | | | | | 4782) |
| A029400060 | A0294041A11 | 9GS | A0294041A11 | | | FLAG3-HIS6 |
| | | | | | | (SEQ ID NO: |
| | | | | | | 4782) |
| A029400163 | A0294040C12 | 9GS | A0294040C12 | 9GS | ALB23002 | FLAG3-HIS6 |
| | | | | | | (SEQ ID NO: |
| | | | | | | 4782) |
| A029400059 | A0294040C12 | 9GS | A0294040C12 | | | FLAG3-HIS6 |
| | | | | | | (SEQ ID NO: |
| | | | | | | 4782) |
| A029400067 | A0294047E02 | 35GS | A0294047E02 | 9GS | ALB23002 | FLAG3-HIS6 |
| | | | | | | (SEQ ID NO: |
| | | | | | | 4782) |
| A029400068 | A0294047E02 | 9GS | A0294047E02 | 9GS | PCRV | FLAG3- |
| | | | | | | HIS6(SEQ ID |
| | | | | | | NO: 4782) |
| A029400069 | IRR | 9GS | A0294047E02 | 9GS | ALB23002 | FLAG3-HIS6 |
| | | | | | | (SEQ ID NO: |
| | | | | | | 4782) |
| A029400070 | A0294047E02 | 9GS | IRR | 9GS | ALB23002 | FLAG3-HIS6 |
| | | | | | | (SEQ ID NO: |
| | | | | | | 4782) |

IRR: irrelevant ISVD that does not bind PD-1

### 5.17.2 Effect of Formatting in the TT-Specific Co-Culture Assay

The formatted construct of the 4 leads molecules with and without an anti-albumin ISVD were evaluated in a TT assay (as described 6.7). The potency of the monovalent leads and their corresponding format with and without an anti-albumin ISVD were compared with a benchmark molecule. Results are given in Table B-8.

TABLE B-8
EC50 values in the TT assay of monovalent ISVDs
and ISVDs formatted with an anti-albumin ISVD

| | | | | | fold dif | bivalent with anti- |
|---|---|---|---|---|---|---|
| | EC50 (M) IFN-Gamma production | | | | Benchmark/ | ALB/ bivalent w/o |
| | D1479 | D1550 | D1552 | Average | ISVD | anti-ALB |
| Benchmark1 | 2.62E−09 | 3.00E−09 | 2.02E−09 | 2.51E−09 | 1.00 | |
| A029400058 | 2.27E−09 | 1.69E−09 | ND | 2.0E−09 | 1.28 | 2.61 |
| A029400063 | 9.05E−09 | 7.53E−09 | ND | 8.3E−09 | 0.30 | 10.99 |
| A029400064 | 2.48E−09 | 2.67E−09 | 2.78E−09 | 2.6E−09 | 0.95 | 3.52 |
| A029400062 | 6.35E−10 | 7.76E−10 | 8.59E−10 | 7.5E−10 | 3.35 | 1.00 |
| A029400067 | 2.61E−09 | 1.51E−09 | 1.37E−09 | 1.8E−09 | 1.43 | 2.34 |
| A029400069 | no fit | no fit | no fit | no fit | | |
| A029400070 | 2.45E−08 | 1.63E−08 | 1.13E−08 | 1.7E−08 | 0.15 | |
| A029400068 | 5.87E−10 | 7.34E−10 | 5.85E−10 | 6.3E−10 | 3.98 | 0.84 |
| A0294047E02 | 4.26E−09 | 2.78E−09 | 2.84E−09 | 3.2E−09 | 0.78 | |
| A029400065 | 1.25E−09 | 6.21E−10 | 9.17E−10 | 8.9E−10 | 2.81 | 2.36 |
| A029400061 | 3.26E−10 | 2.48E−10 | 6.73E−10 | 3.8E−10 | 6.63 | 1.00 |
| A0294037F03 | 2.92E−08 | 3.39E−08 | 3.03E−08 | 3.1E−08 | 0.08 | |
| A029400066 | 1.05E−08 | 8.87E−09 | 6.54E−09 | 8.5E−09 | 0.30 | 5.00 |
| A029400060 | 1.87E−09 | 1.33E−09 | 1.96E−09 | 1.7E−09 | 1.48 | 1.00 |
| A0294041A11 | 4.27E−08 | 7.63E−08 | 1.10E−07 | 7.1E−08 | 0.04 | |
| A029400163 | 1.11E−08 | 1.58E−08 | 3.41E−09 | 8.4E−09 | 0.30 | 2.81 |
| A029400059 | 2.68E−09 | 5.49E−09 | 1.83E−09 | 3.0E−09 | 0.84 | 1.00 |
| A0294040C12 | 7.82E−08 | 4.17E−08 | 6.03E−08 | 5.8E−08 | 0.04 | |

For the four ISVD molecule leads in format with anti-albumin ISVD, a reduction of potency is observed. No significant effect was observed when using a longer linker length between anti-PD-1 ISVD building blocks (A029400067). Interestingly, all leads in trivalent format (i.e., bivalent for PD-1 binding and additionally including anti-albumin ISVD) have similar potency and efficacy as Benchmark1.

### 5.18 Example 9: Sequence Optimization of ISVD A0294047E02

### 5.18.1 Sequence Optimization

ISVD A0294047E02 was further sequence optimized to improve chemical stability, to reduce binding by pre-existing antibodies to the ISVD and to make the ISVD more human like. The optimized sequences are depicted in Tables A-2.

### 5.18.2 Off-Rate Analysis of the Sequence Optimized ISVDs

The sequence optimized ISVDs were subjected to off-rate analysis towards human and cyno PD-1 by Surface Plasmon Resonance (SPR) on a ProteOn XPR36 (Bio-Rad Laboratories), SPR-32 Bruker and an Cytiva Biacore 8K+ instrument. The experiment was performed at 25° C. or 37° C. Thereto, recombinant hPD-1::Halotag-histag and cPD-1::Halotag-histag (in house produced) were immobilized on GLC sensorchip (Bio-Rad Laboratories), or a High-Capacity Amine chip (Bruker) or on a CM5 chip (GE Healthcare Life Sciences) via amine coupling, using EDC and NHS chemistry (running buffer used: HBS-EP+, pH7.4). Periplasmic extracts were diluted 1:10 in running buffer (HBS-EP+, pH7.4) and they were injected over the sensor surfaces (association: 120 s, dissociation: 900 s, 45 μL/min). Between sample injections, the surfaces were regenerated with 100 mM HCL (Merck Millipore). Several buffer blanks were injected for double referencing. Data was double referenced by subtracting a reference ligand lane and a blank buffer injection. Processed sensorgrams were analysed based on the 1:1 interaction model (Langmuir binding model) using ProteOn Manager 3.1.0 software (Bio-Rad Laboratories) or using SPR-32 Analyzer 3.2 analysis software or using the Biacore Insight Evaluation software.

Off rate screenings were also done using SPR based assays on a Bruker SPR-32 instrument, a Bio-Rad ProteOn XPR-36 instrument and an Cytiva Biacore 8K+ instrument. ISVDs were capture via the directly immobilized anti-FLAG M2 antibody (Sigma-Aldrich/SLCG2330) on a High-Capacity Amine chip (Bruker), a GLH chip (Bio-Rad) or on a CM5 chip (GE Healthcare Life Sciences). Human PD-1 His (R&D Systems/8986-PD) and cyno PD-1 His (R&D Systems/8509-PD) were flowing over the captured ISVDs. Capture of ISVDs from periplamic extract at 1:10 dilution was performed for association: 120 s, at 10 μL/min, followed by protein injection at 200 nM in running buffer (1×HBS-EP+, pH7.4) over the captured surface (association: 120 s, dissociation: 600 s-900 s, 45 μL/min). Data was double referenced by subtracting a reference ligand lane and a blank buffer injection. Processed sensorgrams were analysed based on the 1:1 interaction model (Langmuir binding model) using the Sierra Analyser analysis software, the ProteOn Manager analysis software or using the Biacore Insight Evaluation software.

The off-rate values for the sequence optimized variants are depicted in Tables B-9 and B-10.

TABLE B-9
Off-rate values for the sequence optimized variants of ISVD
A0294047E02 measured at 25° C.

| | cPD-1 | hPD-1 | cPD-1 | hPD-1 |
|---|---|---|---|---|
| | koff (1/s) | koff (1/s) | koff (1/s) | koff (1/s) |
| | Halotag- | Halotag- | cPD-1 | hPD-1 |
| | histag | histag | His-tag | Histag |
| A0294047E02 | 3.80E−04 | 2.90E−04 | | |
| A029403867 | 4.70E−03 | 5.70E−03 | | |
| A029403869 | 1.30E−03 | 1.80E−03 | | |
| A029401559 | 4.10E−04 | 4.60E−04 | | |
| A029401558 | 7.80E−05 | 1.10E−04 | | |
| A029401557 | 1.50E−04 | 1.70E−04 | | |
| A029401556 | 1.30E−04 | 1.30E−04 | | |
| A029401555 | 2.00E−04 | 2.20E−04 | | |
| A029401554 | 1.50E−04 | 1.40E−04 | | |
| A029401540 | 3.90E−04 | 3.60E−04 | | |
| A029401538 | 4.10E−04 | 3.60E−04 | | |
| A029401536 | 3.60E−04 | 3.80E−04 | | |
| A029401535 | 3.90E−04 | 4.30E−04 | | |
| A029401562 | 4.90E−04 | 6.00E−04 | | |
| A029401534 | 3.20E−04 | 3.20E−04 | | |
| A029401531 | 3.20E−04 | 3.80E−04 | | |
| A029401529 | 3.00E−04 | 3.30E−04 | | |
| A029401528 | 1.30E−04 | 1.20E−04 | | |
| A029401527 | 1.30E−04 | 1.50E−04 | | |
| A029401525 | 2.30E−04 | 2.10E−04 | | |
| A029401524 | 6.60E−04 | 8.50E−04 | | |
| A029401523 | 2.50E−04 | 2.20E−04 | | |
| A029401514 | 2.10E−04 | 3.20E−04 | | |
| A029401511 | 3.40E−04 | 4.30E−04 | | |
| A029401504 | 2.10E−04 | 3.30E−04 | | |
| A029401501 | 2.00E−04 | 3.20E−04 | | |
| A029401500 | 2.10E−04 | 3.10E−04 | | |
| A029401533 | 3.30E−04 | 3.60E−04 | | |
| A029401563 | 9.80E−04 | 1.00E−03 | | |
| A029401564 | 2.60E−03 | 2.00E−03 | | |
| A029401570 | 8.00E−04 | 1.40E−03 | | |
| A029401640 | 3.70E−04 | 6.30E−04 | | |
| A029401639 | 5.80E−04 | 1.00E−03 | | |
| A029401638 | 6.50E−04 | 1.00E−03 | | |
| A029401635 | 1.80E−04 | 2.30E−04 | | |
| A029401634 | 1.30E−03 | 1.90E−03 | | |
| A029401633 | 3.40E−04 | 5.90E−04 | | |
| A029401632 | 4.60E−04 | 7.20E−04 | | |
| A029401631 | 6.20E−04 | 1.10E−03 | | |
| A029401622 | 2.00E−04 | 2.60E−04 | | |
| A029401616 | 2.70E−04 | 4.20E−04 | | |
| A029401610 | 3.70E−04 | 5.60E−04 | | |
| A029401609 | 2.00E−04 | 2.40E−04 | | |
| A029401608 | 6.40E−04 | 9.70E−04 | | |
| A029401604 | 3.60E−04 | 6.00E−04 | | |
| A029401603 | 1.30E−03 | 2.20E−03 | | |
| A029401601 | 3.40E−04 | 5.40E−04 | | |
| A029401600 | 2.80E−04 | 4.40E−04 | | |
| A029401599 | 5.50E−04 | 6.80E−04 | | |
| A029401597 | 2.90E−04 | 4.40E−04 | | |
| A029401596 | 3.50E−04 | 5.80E−04 | | |
| A029401595 | 3.80E−04 | 6.10E−04 | | |
| A029401592 | 1.90E−03 | 1.20E−03 | | |
| A029401580 | 2.20E−04 | 2.00E−04 | | |
| A029401579 | 3.30E−04 | 2.90E−04 | | |
| A029401575 | 9.50E−05 | 1.30E−04 | | |
| A029401573 | 1.40E−04 | 1.50E−04 | | |
| A029401571 | 5.20E−04 | 5.40E−04 | | |
| A029401498 | 2.10E−04 | 3.30E−04 | | |
| A029401641 | 2.80E−04 | 5.00E−04 | | |
| A029401496 | 1.90E−04 | 3.20E−04 | | |
| A029401493 | 2.50E−04 | 3.60E−04 | | |
| A029405091 | | | 6.48E−05 | 6.18E−03 |
| A029405092 | | | 5.51E−04 | 4.63E−03 |
| A029405093 | | | 1.45E−04 | 1.86E−03 |
| A029405094 | | | 2.98E−04 | 1.19E−04 |
| A029405095 | | | 5.34E−05 | 4.74E−04 |
| A029405096 | | | 4.59E−04 | 4.47E−04 |
| A029405097 | | | 9.75E−04 | nd |
| A029405098 | | | 9.59E−04 | 4.03E−04 |
| A029405099 | | | 3.51E−04 | 2.14E−04 |
| A029405100 | | | 4.96E−04 | 1.04E−02 |
| A029405101 | | | 3.14E−04 | 3.53E−04 |
| A029405102 | | | nd | 4.68E−03 |
| A029405103 | | | 4.71E−05 | 3.02E−04 |
| A029405104 | | | 2.71E−05 | 7.39E−05 |
| A029405105 | | | 6.18E−04 | 1.15E−03 |
| A029405106 | | | 5.60E−04 | 5.16E−04 |
| A029405108 | | | 2.51E−03 | 1.06E−04 |
| A029405109 | | | 1.06E−04 | 4.88E−04 |
| A029405110 | | | 9.32E−04 | 1.13E−04 |
| A029405111 | | | 1.17E−03 | 3.44E−04 |
| A029405112 | | | 8.83E−04 | 5.36E−04 |
| A029405113 | | | nd | 1.36E−04 |
| A029405114 | | | 6.38E−04 | 5.83E−05 |
| A029405115 | | | 1.96E−04 | 8.38E−04 |
| A029405116 | | | 6.29E−04 | 8.50E−05 |
| A029405117 | | | 1.14E−03 | 1.29E−04 |
| A029401492 | 2.30E−04 | 3.90E−04 | | |
| A029401491 | 2.50E−04 | 3.90E−04 | | |
| A029401490 | 1.80E−04 | 3.00E−04 | | |
| A029401489 | 1.80E−04 | 2.80E−04 | | |
| A029401488 | 2.00E−04 | 3.10E−04 | | |
| A029401480 | 1.70E−04 | 3.20E−04 | | |
| A029401479 | 2.20E−04 | 3.70E−04 | | |
| A029401474 | 2.40E−04 | 3.80E−04 | | |
| A029401467 | 2.90E−04 | 4.50E−04 | | |
| A029401465 | 2.10E−04 | 3.50E−04 | | |
| A029401464 | 2.50E−04 | 4.00E−04 | | |
| A029401463 | 2.20E−04 | 3.70E−04 | | |
| A029401462 | 2.60E−04 | 3.90E−04 | | |
| A029401461 | 2.20E−04 | 3.60E−04 | | |
| A029401459 | 1.70E−04 | 3.20E−04 | | |
| A029401458 | 2.10E−04 | 3.50E−04 | | |
| A029401457 | 1.90E−04 | 3.40E−04 | | |
| A029401456 | 2.00E−04 | 3.30E−04 | | |
| A029401455 | 2.00E−03 | 3.20E−03 | | |
| A029401454 | 1.70E−04 | 3.10E−04 | | |
| A029405118 | | | 1.05E−04 | 1.99E−04 |
| A029405119 | | | 4.70E−04 | 2.85E−04 |
| A029405121 | | | 1.50E−04 | 3.05E−01 |
| A029405122 | | | 1.70E−04 | 4.04E−05 |
| A029405123 | | | 1.98E−04 | 3.81E−04 |
| A029405124 | | | 1.08E−04 | 2.91E−04 |
| A029401495 | 1.90E−04 | 3.10E−04 | | |
| A029401642 | 4.00E−04 | 7.50E−04 | | |
| A029401561 | 1.60E−04 | 2.00E−04 | | |
| A029401560 | 5.80E−04 | 6.20E−04 | | |
| A029401643 | 4.70E−04 | 5.90E−04 | | |
| A029405125 | | | 2.57E−05 | 1.82E−03 |
| A029405126 | | | 2.88E−05 | 7.12E−04 |
| A029405127 | | | 2.34E−03 | 2.41E−05 |
| A029405128 | | | 4.83E−04 | 7.36E−05 |
| A029405129 | | | 1.19E−03 | 1.08E−03 |
| A029405130 | | | 4.72E−04 | 1.18E−04 |
| A029405131 | | | 6.67E−05 | 8.55E−05 |
| A029405132 | | | 1.62E−03 | 5.05E−04 |
| A029405133 | | | 1.91E−05 | 1.34E−03 |
| A029405134 | | | 1.04E−03 | 2.43E−04 |
| A029405135 | | | 1.80E−04 | 9.17E−05 |
| A029405136 | | | 5.00E−01 | 6.65E−05 |
| A029404008 | 5.10E−03 | 8.10E−03 | | |
| A029403984 | 1.50E−03 | 2.50E−03 | | |
| A029403981 | 1.10E−03 | 1.00E−03 | | |
| A029403973 | 1.90E−04 | 2.60E−04 | | |
| A029403967 | 6.30E−04 | 7.40E−04 | | |
| A029403964 | 1.70E−04 | 3.00E−04 | | |
| A029403962 | 2.70E−03 | 4.70E−03 | | |
| A029403957 | 5.30E−03 | 1.60E−03 | | |
| A029405137 | | | 1.00E−04 | 3.57E−04 |
| A029403949 | 5.50E−04 | 6.30E−04 | | |
| A029403945 | 3.10E−04 | 2.70E−04 | | |
| A029403943 | 3.90E−04 | 5.20E−04 | | |
| A029405138 | | | 1.56E−03 | 2.00E−03 |
| A029405139 | | | 1.58E−03 | 3.21E−04 |
| A029405140 | | | 4.31E−04 | 3.18E−04 |
| A029405141 | | | 9.22E−04 | 4.47E−04 |
| A029405142 | | | 1.26E−05 | 4.58E−04 |
| A029405143 | | | 1.21E−03 | 6.92E−04 |
| A029405144 | | | 9.48E−04 | 4.10E−04 |
| A029405145 | | | 2.75E−04 | nd |
| A029405146 | | | 1.06E−03 | nd |
| A029405147 | | | 1.26E−04 | nd |
| A029405149 | | | 3.68E−04 | nd |
| A029405153 | | | 6.16E−05 | 4.21E−04 |
| A029403946 | 1.20E−03 | 1.70E−03 | | |
| A029401645 | 7.30E−04 | 1.30E−03 | | |
| A029401673 | 3.20E−03 | 3.10E−03 | | |
| A029403862 | 4.50E−03 | 1.50E−03 | | |
| A029403861 | 6.30E−04 | 6.00E−04 | | |
| A029403860 | 2.20E−02 | 3.60E−03 | | |
| A029401711 | 1.30E−03 | 1.60E−03 | | |
| A029401701 | 2.20E−03 | 1.90E−03 | | |
| A029401698 | 1.40E−03 | 1.70E−03 | | |
| A029401691 | 2.70E−04 | 3.50E−04 | | |
| A029401687 | 1.50E−03 | 2.80E−03 | | |
| A029401686 | 2.20E−03 | 3.00E−03 | | |
| A029401684 | 6.00E−04 | 4.80E−04 | | |
| A029401680 | 2.80E−03 | 3.70E−03 | | |
| A029401675 | 3.80E−04 | 5.30E−04 | | |
| A029403865 | 5.60E−04 | 8.00E−04 | | |
| A029401672 | 9.00E−04 | 9.40E−04 | | |
| A029401670 | 1.70E−03 | 2.00E−03 | | |
| A029401669 | 8.70E−04 | 9.90E−04 | | |
| A029401667 | 5.80E−05 | 5.10E−05 | | |
| A029401665 | 7.60E−04 | 9.50E−04 | | |
| A029401664 | 1.50E−04 | 2.80E−04 | | |
| A029401663 | 1.30E−04 | 2.30E−04 | | |
| A029401662 | 1.30E−03 | 2.00E−03 | | |
| A029401658 | 1.50E−03 | 2.00E−03 | | |
| A029401649 | 7.00E−04 | 1.10E−03 | | |
| A029401648 | 3.60E−04 | 4.60E−04 | | |
| A029401646 | 6.50E−04 | 1.10E−03 | | |
| A029403866 | 3.50E−04 | 4.30E−04 | | |
| A029403871 | 2.40E−04 | 2.10E−04 | | |
| A029401674 | 1.10E−03 | 1.30E−03 | | |
| A029403864 | 4.60E−04 | 4.40E−04 | | |
| A029403941 | 2.30E−04 | 3.30E−04 | | |
| A029403940 | 4.50E−04 | 6.70E−04 | | |
| A029403939 | 7.80E−04 | 9.90E−04 | | |
| A029403937 | 4.00E−04 | 5.00E−04 | | |
| A029403935 | 1.80E−02 | nd | | |
| A029403934 | 7.90E−04 | 1.10E−03 | | |
| A029403925 | 7.40E−04 | 5.70E−04 | | |
| A029403916 | 7.50E−04 | 6.30E−04 | | |
| A029403912 | 8.00E−04 | 8.90E−04 | | |
| A029403904 | 2.10E−04 | 1.00E−03 | | |
| A029403903 | 8.50E−04 | 1.10E−03 | | |
| A029403896 | 2.80E−03 | 1.30E−03 | | |
| A029403895 | 4.50E−04 | 3.70E−04 | | |
| A029403894 | 1.90E−03 | 1.50E−03 | | |
| A029403892 | 2.50E−03 | 2.40E−03 | | |
| A029403889 | 2.60E−03 | 1.80E−03 | | |
| A029403888 | 1.50E−03 | 1.40E−03 | | |
| A029403884 | 1.60E−03 | 1.00E−03 | | |
| A029403880 | 6.40E−04 | 8.70E−04 | | |
| A029403879 | 2.30E−03 | 2.30E−03 | | |
| A029403875 | 4.80E−04 | 5.20E−04 | | |
| A029403874 | 6.20E−04 | 5.30E−04 | | |
| A029403873 | 6.50E−04 | 5.40E−04 | | |
| A029403872 | 4.50E−04 | 3.10E−04 | | |
| A029403870 | 1.10E−03 | 1.50E−03 | | |

TABLE B-10
Off-rate values for the sequence optimized variants of
ISVD A0294047E02 measured at 37° C.

| | cPD-1 | hPD-1 |
|---|---|---|
| ISVD ID | koff (1/s) | koff (1/s) |
| A029400076 | 5.49E−04 | 6.14E−04 |
| A029400077 | 4.31E−04 | 3.75E−04 |
| A029400078 | 4.85E−04 | 4.14E−04 |
| A029400079 | 2.42E−04 | 2.63E−04 |
| A029400080 | 2.39E−04 | 2.24E−04 |
| A029400081 | 1.70E−04 | 1.65E−04 |
| A029400082 | 1.56E−04 | 1.10E−04 |
| A029400083 | 3.94E−04 | 2.51E−04 |
| A029400084 | 8.34E−04 | 7.43E−04 |
| A029400086 | 2.78E−04 | 2.42E−04 |
| A029400087 | 6.28E−04 | 7.20E−04 |
| A029400088 | 8.10E−04 | 6.91E−04 |
| A029400089 | 7.19E−04 | 6.91E−04 |
| A029400090 | 3.28E−04 | 2.72E−04 |
| A029400091 | 4.14E−04 | 2.61E−04 |
| A029400092 | 3.44E−04 | 2.37E−04 |
| A029400093 | 2.48E−04 | 1.76E−04 |
| A029400094 | 2.65E−04 | 2.37E−04 |
| A029400096 | 2.13E−04 | 1.71E−04 |
| A029400098 | 3.12E−04 | 2.57E−04 |
| A029400099 | 5.87E−04 | 4.07E−04 |
| A029400100 | 7.95E−04 | 7.52E−04 |
| A029400101 | 2.26E−04 | 1.68E−04 |
| A029400102 | 4.28E−04 | 2.92E−04 |
| A029400103 | 1.04E−04 | 8.59E−05 |
| A029400162 | 2.18E−04 | 1.48E−04 |
| A029400164 | 8.74E−04 | 7.60E−04 |
| A029400165 | 4.23E−04 | 3.78E−04 |
| A029400166 | 3.76E−04 | 2.83E−04 |
| A029400167 | 3.35E−04 | 2.29E−04 |
| A029400199 | 3.21E−04 | 1.98E−04 |
| A029404710 | 3.58E−04 | 7.27E−04 |
| A029404711 | 4.54E−04 | 8.39E−04 |
| A029404713 | 6.41E−05 | 8.42E−05 |
| A029404714 | 6.47E−04 | 1.22E−03 |
| A029404715 | 6.54E−04 | 1.21E−03 |
| A029400085 | 1.69E−04 | 1.70E−04 |
| A029404717 | 6.17E−05 | 6.91E−05 |
| A029404718 | 4.92E−04 | 8.87E−04 |
| A029404719 | 1.91E−04 | 3.10E−04 |
| A029404720 | 8.07E−05 | 1.02E−04 |
| A029404721 | 5.36E−04 | 1.01E−03 |
| A029404722 | 2.09E−04 | 3.54E−04 |
| A029404723 | 1.30E−02 | 1.31E−02 |
| A029404725 | 8.51E−04 | 1.07E−03 |
| A029404726 | 2.63E−04 | 4.15E−04 |
| A029404727 | 4.01E−04 | 5.71E−04 |
| A029404728 | 3.60E−04 | 5.58E−04 |
| A029404729 | 8.06E−05 | 1.08E−04 |
| A029404730 | 1.38E−04 | 1.61E−04 |
| A029404731 | 7.12E−05 | 9.18E−05 |
| A029404732 | 3.07E−04 | 4.90E−04 |
| A029404733 | 4.15E−04 | 6.24E−04 |
| A029404734 | 3.40E−04 | 5.52E−04 |
| A029404735 | 4.32E−04 | 7.61E−04 |
| A029404736 | 5.44E−04 | 9.84E−04 |
| A029404737 | 2.28E−04 | 4.00E−04 |
| A029404738 | 1.47E−02 | 1.38E−02 |
| A029404740 | 8.41E−04 | 1.02E−03 |
| A029404741 | 5.07E−04 | 9.51E−04 |
| A029404742 | 6.09E−04 | 1.13E−03 |
| A029404743 | 3.55E−04 | 5.41E−04 |
| A029404744 | 1.55E−04 | 2.80E−04 |
| A029404745 | 3.75E−04 | 6.49E−04 |
| A029404870 | 7.75E−04 | 1.68E−03 |
| A029404871 | 8.20E−04 | 1.75E−03 |
| A029404872 | 9.96E−04 | 2.07E−03 |
| A029404873 | 7.35E−04 | 1.55E−03 |
| A029404874 | 7.04E−04 | 1.49E−03 |
| A029404875 | 2.09E−03 | 1.12E−03 |
| A029404876 | 1.57E−03 | 8.97E−04 |
| A029404877 | 6.46E−04 | 1.26E−03 |
| A029404878 | 7.48E−04 | 1.55E−03 |
| A029404879 | 8.37E−04 | 1.73E−03 |
| A029404880 | 7.56E−04 | 1.61E−03 |
| A029404881 | 8.15E−04 | 1.71E−03 |
| A029404882 | 4.00E−04 | 8.89E−04 |
| A029404883 | 1.30E−03 | 1.02E−03 |
| A029404885 | 3.57E−04 | 5.27E−04 |
| A029404886 | 4.15E−04 | 4.84E−04 |
| A029404887 | 6.21E−04 | 5.57E−04 |
| A029404888 | 7.36E−04 | 6.05E−04 |
| A029404889 | 3.14E−04 | 4.77E−04 |
| A029404890 | 5.70E−04 | 5.71E−04 |
| A029404891 | 6.54E−04 | 1.30E−03 |
| A029404892 | 1.12E−03 | 1.18E−03 |
| A029404893 | 5.14E−04 | 9.69E−04 |
| A029404895 | 1.25E−03 | 1.38E−03 |
| A029404896 | 1.04E−03 | 1.05E−03 |
| A029404897 | 8.62E−04 | 1.67E−03 |
| A029404898 | 7.38E−04 | 1.42E−03 |
| A029404899 | 9.45E−04 | 1.52E−03 |
| A029404900 | 7.54E−04 | 1.22E−03 |
| A029404901 | 5.46E−04 | 1.17E−03 |
| A029404903 | 1.48E−03 | 2.26E−03 |
| A029404904 | 1.12E−03 | 1.43E−03 |
| A029404905 | 8.83E−04 | 1.84E−03 |
| A029404906 | 5.31E−04 | 7.25E−04 |
| A029404907 | 4.89E−04 | 4.40E−04 |
| A029404910 | 6.93E−04 | 8.05E−04 |
| A029404912 | nd | 1.42E−03 |
| A029404913 | 1.14E−03 | 1.33E−03 |
| A029404914 | 8.15E−04 | 1.18E−03 |
| A029404915 | 9.82E−04 | 9.07E−04 |
| A029404916 | 1.20E−03 | 1.06E−03 |
| A029404917 | 7.38E−04 | 9.29E−04 |
| A029404918 | 8.65E−04 | 1.36E−03 |
| A029404920 | 7.91E−04 | 8.07E−04 |
| A029404921 | 8.21E−04 | 9.10E−04 |
| A029404922 | 3.42E−04 | 6.65E−04 |
| A029404923 | 5.72E−04 | 9.22E−04 |
| A029404924 | 5.87E−04 | 8.23E−04 |
| A029404925 | 2.47E−04 | 5.11E−04 |
| A029404926 | 2.95E−04 | 5.66E−04 |
| A029404927 | 9.04E−04 | 8.08E−04 |
| A029404929 | 2.83E−04 | 5.93E−04 |
| A029404930 | 5.66E−04 | 6.77E−04 |
| A029404931 | 6.07E−04 | 6.02E−04 |
| A029404932 | 2.60E−04 | 4.77E−04 |
| A029404933 | 3.55E−04 | 7.19E−04 |
| A029404934 | 4.92E−04 | 9.97E−04 |
| A029404937 | 8.62E−04 | 7.16E−04 |
| A029404938 | 9.75E−04 | 7.74E−04 |
| A029404941 | 5.76E−04 | 5.67E−04 |
| A029404942 | 5.88E−04 | 5.22E−04 |
| A029404944 | 6.26E−04 | 6.95E−04 |
| A029404946 | 8.79E−04 | 6.88E−04 |
| A029404947 | 7.52E−04 | 7.24E−04 |
| A029404948 | 8.45E−04 | 6.75E−04 |
| A029404952 | 7.06E−04 | 7.11E−04 |
| A029404956 | nd | 8.83E−04 |
| A029404957 | 1.19E−03 | nd |
| A029404958 | 1.06E−03 | 7.65E−04 |
| A029404959 | 6.41E−04 | 6.33E−04 |
| A029404960 | 7.79E−04 | 6.27E−04 |
| A029404961 | 9.14E−04 | 6.40E−04 |
| A029404962 | 1.03E−03 | 7.53E−04 |
| A029404963 | 1.03E−03 | 7.72E−04 |
| A029404964 | 8.42E−04 | 7.90E−04 |
| A029404966 | 1.21E−03 | 7.99E−04 |
| A029404967 | 8.56E−04 | 7.45E−04 |
| A029404968 | 3.66E−04 | 6.85E−04 |
| A029404969 | 3.62E−04 | 6.71E−04 |
| A029404970 | 3.08E−04 | 5.75E−04 |
| A029404972 | 3.42E−04 | 6.26E−04 |
| A029404973 | 3.29E−04 | 6.17E−04 |
| A029404974 | 3.78E−04 | 7.39E−04 |
| A029404975 | 3.33E−04 | 6.14E−04 |
| A029404976 | 3.45E−04 | 6.06E−04 |
| A029404977 | 3.82E−04 | 6.73E−04 |
| A029404978 | 2.96E−04 | 5.67E−04 |
| A029404979 | 4.08E−04 | 6.35E−04 |
| A029404980 | 4.09E−04 | 5.49E−04 |
| A029404982 | 4.73E−04 | 9.87E−04 |
| A029404983 | nd | 5.25E−04 |
| A029404985 | 4.34E−04 | 8.17E−04 |
| A029404986 | 8.87E−04 | 1.43E−03 |
| A029404987 | 7.58E−04 | 1.05E−03 |
| A029404988 | 3.53E−04 | 6.83E−04 |
| A029404989 | 3.13E−04 | 5.48E−04 |
| A029404990 | 5.23E−04 | 7.04E−04 |
| A029404992 | 5.00E−04 | 9.05E−04 |
| A029404993 | 8.96E−04 | 9.22E−04 |
| A029404994 | 9.21E−04 | 8.70E−04 |
| A029404995 | 4.87E−04 | 9.31E−04 |
| A029404996 | 3.77E−04 | 7.37E−04 |
| A029404997 | 9.62E−04 | 1.54E−03 |
| A029404998 | 9.84E−04 | 1.42E−03 |
| A029404999 | 4.11E−04 | 7.40E−04 |
| A029405000 | 1.08E−03 | 1.07E−03 |
| A029405001 | 8.29E−04 | 7.35E−04 |
| A029405002 | 8.18E−04 | 6.60E−04 |
| A029405003 | 5.09E−04 | 5.35E−04 |
| A029405005 | 7.58E−04 | 7.30E−04 |
| A029405006 | 8.76E−04 | 7.34E−04 |
| A029405007 | 6.45E−04 | 7.68E−04 |
| A029405008 | 9.87E−04 | 6.68E−04 |
| A029405009 | 7.67E−04 | 7.90E−04 |
| A029405010 | 8.33E−04 | 7.48E−04 |
| A029405011 | 9.26E−04 | 6.41E−04 |
| A029405012 | 8.51E−04 | 5.38E−04 |
| A029405013 | 4.87E−04 | 4.60E−04 |
| A029405014 | 8.79E−04 | 6.12E−04 |
| A029405016 | 1.13E−03 | nd |
| A029405017 | 7.67E−04 | 6.18E−04 |
| A029405018 | 6.62E−04 | 6.93E−04 |
| A029405019 | 8.44E−04 | 6.38E−04 |
| A029405021 | 7.26E−04 | 7.27E−04 |
| A029405024 | 1.00E−03 | 1.82E−03 |
| A029405025 | 6.68E−04 | 1.41E−03 |
| A029405026 | 3.78E−04 | 5.79E−04 |
| A029405027 | 9.57E−04 | 1.30E−03 |
| A029405028 | 9.85E−04 | 7.57E−04 |
| A029405029 | 4.92E−04 | 8.52E−04 |
| A029405030 | 3.70E−04 | 7.08E−04 |
| A029405031 | 3.76E−04 | 7.30E−04 |
| A029405032 | 3.57E−04 | 7.15E−04 |
| A029405033 | 4.72E−04 | 6.91E−04 |
| A029405034 | 7.77E−04 | 7.66E−04 |
| A029405035 | 1.05E−03 | 7.48E−04 |
| A029400095 | 6.64E−04 | 8.12E−04 |
| A029400168 | 3.13E−04 | 2.98E−04 |
| A029404716 | 2.76E−04 | 4.67E−04 |

nd: not determined

The sequence optimized ISVDs kept a good off-rate values (kd) in the range of about 1E-03 to 1E-04 or even 1E-05 1/sec.

### 5.18.3 Biophysical Characterization of the Sequence Optimized ISVDs

The biophysicial characteristics of the sequence optimized ISVDs of ISVD A0294047E02 were tested in thermal shift assay (Tm) and a temperature of onset of aggregation assay (Tagg).

The thermal shift assay and Tagg assay were performed as described in example 6.5.1 and 6.5.4 respectively. The Tm and Tagg values obtained for the sequence optimized variants are shown in Table B-11.

TABLE B-11
Biophysical properties of the sequence optimized
variants of ISVD A0294047E02

| | Tm | Tagg |
|---|---|---|
| ISVD ID | (° C.) | (° C.) |
| A0294047E02 | 75.9 | nd |
| A029400082 | 66.2 | 63 |
| A029400092 | 60.2 | 56.5 |
| A029400096 | 70.4 | 65.4 |
| A029400101 | 70.6 | 66.4 |
| A029400103 | 72.8 | 68.4 |
| A029400162 | 72.6 | 68.7 |
| A029400166 | 68 | 63.8 |
| A029404991 | 63.8 | nd |
| A029400085 | 79 | 76 |
| A029404935 | 64.2 | nd |
| A029404940 | 62.3 | nd |
| A029404928 | 57.0 | nd |
| A029405120 | 69 | nd |
| A029404936 | 58.4 | nd |
| A029404939 | 57.4 | nd |
| A029404943 | 60.4 | nd |
| A029404971 | 60.8 | nd |
| A029404981 | 62.8 | nd |
| A029405107 | 65.5 | nd |
| A029405040 | 71.3 | nd |
| A029405095 | 62 | nd |
| A029405104 | 59 | nd |
| A029405109 | 65 | nd |
| A029405110 | 69 | nd |
| A029405117 | 70 | nd |
| A029405118 | 67 | nd |
| A029405121 | 59 | nd |
| A029404013 | 78.8 | nd |
| A029405126 | 64 | nd |
| A029405128 | 65 | nd |
| A029405129 | 71 | nd |
| A029405130 | 71 | nd |
| A029404014 | 74.9 | nd |
| A029404021 | 74.9 | nd |
| A029404029 | 79.3 | nd |
| A029404031 | 75.4 | nd |
| A029404033 | 74.9 | nd |
| A029404035 | 74.9 | nd |
| A029404712 | 70.6 | nd |
| A029405140 | 61 | nd |
| A029405142 | 62 | nd |
| A029405143 | 65 | nd |
| A029404716 | 64 | nd |

nd: not determined

### 5.18.4 Characterization of the Sequence Optimized ISVDs for Binding Cells Expressing PD-1

The sequence optimized variants of ISVD A0294047E02 were tested for FACS binding on CHO cell lines and PreB 300-19 cells expressing human or cyno PD-1 (in house made). The binding FACS was carried out as described in Example 6.1.3. Results are shown in Tables B-12 and B-13.

TABLE B-12
FACS binding of the sequence optimized variants
of ISVD A0294047E02 on CHO cells expressing
human or cyno PD-1

| | EC50 | EC50 |
|---|---|---|
| | (M) | (M) |
| | cPD-1 | hPD-1 |
| A0294047E02 | 2.71E−08 | 1.04E−08 |
| A029400085 | 4.30E−10 | 3.15E−10 |

TABLE B-13
FACS binding of the sequence optimized variants
of ISVD A0294047E02 on PreB 300-19 cells
expressing human or cyno PD-1

| | cyno PD-1 | human PD-1 |
|---|---|---|
| | EC50 | EC50 |
| | (M) | (M) |
| A029400085 | nd | 2.01E−09 |
| A029405120 | 1.29E−08 | 7.77E−09 |
| A029405107 | 1.11E−08 | 8.48E−09 |
| A029405040 | 5.27E−09 | 5.65E−09 |
| A029405095 | 3.49E−08 | 2.24E−08 |
| A029405104 | 7.68E−09 | 4.51E−09 |
| A029405109 | 5.56E−09 | 4.26E−09 |
| A029405110 | 1.68E−08 | 1.05E−08 |
| A029405117 | 4.77E−08 | 5.46E−08 |
| A029405118 | 2.04E−08 | 1.07E−08 |
| A029405121 | 1.61E−08 | 9.89E−09 |
| A029405126 | 1.24E−08 | 6.81E−09 |
| A029405128 | 1.29E−08 | 7.77E−09 |
| A029405129 | 2.29E−08 | 1.51E−08 |
| A029405130 | 9.84E−09 | 6.43E−09 |
| A029405140 | 9.59E−09 | 9.03E−09 |
| A029405142 | 5.54E−09 | 3.37E−09 |
| A029405143 | 1.24E−08 | 9.50E−09 |

nd: not determined

### 5.18.5 Affinity Determination of Sequence Optimized Variants of A0294047E02

Affinity determination of 47E02 ISVD variants was performed as describe in 6.4, but at 37° C. Results are summarized in Table B-14.

TABLE B-14
Affinity determination of the sequence optimization variants of A0294047E02.

| | SPR | | | | | |
|---|---|---|---|---|---|---|
| | Human PD-1 | | | Cyno PD-1 | | |
| Original ID | kon (1/Ms) | koff (1/s) | KD (M) | kon (1/Ms) | koff (1/s) | KD (M) |
| A0294047E02 | 1.89E+05 | 2.06E−04 | 1.09E−09 | 1.37E+05 | 1.03E−04 | 7.56E−10 |
| A029400162 | 1.98E+05 | 2.33E−04 | 1.18E−09 | 1.35E+05 | 1.19E−04 | 8.84E−10 |
| A029400095 | 1.78E+05 | 4.53E−03 | 2.55E−08 | 1.50E+05 | 2.66E−03 | 1.78E−08 |
| A029400168 | 3.06E+05 | 9.97E−04 | 3.26E−09 | 2.09E+05 | 6.04E−04 | 2.88E−09 |

### 5.19 Example 10: Sequence Optimization of ISVD A0294037F03

### 5.19.1 Sequence Optimization

ISVD A0294037F03 was further sequence optimized to improve chemical stability, to reduce binding by pre-existing antibodies to the ISVD and to make the ISVD more human like. The optimized sequences are depicted in Tables A-4.

### 5.19.2 Off-Rate Analysis of the Sequence Optimized ISVDs

The sequence optimized ISVDs were subjected to off-rate analysis towards human and cyno PD-1 by Surface Plasmon Resonance (SPR) on a ProteOn XPR36 (Bio-Rad Laboratories), SPR-32 Bruker and an Cytiva Biacore 8K+ instrument. The experiment was performed at 25° C. or 37° C. Thereto, recombinant hPD1::Halotag-histag and cPD-1::Halotag-histag (in house produced) were immobilized on GLC sensorchip (Bio-Rad Laboratories), or a High-Capacity Amine chip (Bruker) or on a CM5 chip (GE Healthcare Life Sciences) via amine coupling, using EDC and NHS chemistry (running buffer used: HBS-EP+, pH7.4). Periplasmic extracts were diluted 1:10 in running buffer (HBS-EP+, pH7.4) and they were injected over the sensor surfaces (association: 120 s, dissociation: 900 s, 45 μL/min). Between sample injections, the surfaces were regenerated with 100 mM HCL (Merck Millipore). Several buffer blanks were injected for double referencing. Data was double referenced by subtracting a reference ligand lane and a blank buffer injection. Processed sensorgrams were analysed based on the 1:1 interaction model (Langmuir binding model) using ProteOn Manager 3.1.0 software (Bio-Rad Laboratories) or using SPR-32 Analyzer 3.2 analysis software or using the Biacore Insight Evaluation software.

The off-rate values for the sequence optimized variants are depicted in Tables B-15 and B-16.

TABLE B-15
Off-rate values for the sequence optimized variants
of ISVD A0294037F03 measured at 25° C.

| | cPD-1 | hPD-1 |
|---|---|---|
| | koff | koff |
| ISVD ID | (1/s) | (1/s) |
| A029400205 | 1.00E−02 | 5.10E−03 |
| A029400206 | 9.90E−03 | 4.30E−03 |
| A029400207 | 1.30E−02 | 6.50E−03 |
| A029400208 | 1.30E−02 | 6.40E−03 |
| A029400210 | 6.40E−03 | 3.60E−03 |
| A029400212 | 3.90E−03 | 2.70E−03 |
| A029400214 | 3.40E−03 | 2.60E−03 |
| A029400217 | 3.30E−03 | 2.40E−03 |
| A029400221 | 3.80E−03 | nd |
| A029400222 | 3.60E−03 | 2.80E−03 |
| A029400228 | 3.40E−03 | 2.50E−03 |
| A029400236 | 2.80E−03 | 2.20E−03 |
| A029400244 | 2.40E−03 | 1.60E−03 |
| A029400245 | 2.50E−03 | 1.50E−03 |
| A029400246 | 2.10E−03 | nd |
| A029400248 | 2.10E−03 | 1.40E−03 |
| A029400249 | 2.40E−03 | nd |
| A029400253 | 2.10E−03 | 1.40E−03 |
| A029400254 | 2.00E−03 | nd |
| A029400256 | 1.80E−03 | 1.40E−03 |
| A029400262 | 2.10E−03 | 1.40E−03 |
| A029400268 | 2.30E−03 | 1.50E−03 |
| A029400276 | 2.30E−03 | 1.50E−03 |
| A029400281 | 3.50E−03 | 2.60E−03 |
| A029400282 | 6.30E−03 | 4.40E−03 |
| A029400283 | 4.50E−03 | 3.50E−03 |
| A029400284 | 3.30E−03 | 2.30E−03 |
| A029400285 | 5.20E−03 | 4.20E−03 |
| A029400286 | 3.70E−03 | 2.70E−03 |
| A029400287 | 5.50E−03 | 4.10E−03 |
| A029400288 | 3.70E−03 | 2.90E−03 |
| A029400289 | 5.00E−03 | 4.10E−03 |
| A029400294 | 4.40E−03 | 3.50E−03 |
| A029400296 | 5.80E−03 | 4.50E−03 |
| A029400306 | 4.30E−03 | 3.50E−03 |
| A029400319 | 1.30E−03 | 8.70E−04 |
| A029400320 | 1.40E−03 | 9.00E−04 |
| A029400321 | 1.50E−03 | 9.20E−04 |
| A029400322 | 1.90E−03 | 1.20E−03 |
| A029400323 | 1.50E−03 | 9.40E−04 |
| A029400324 | 1.30E−03 | 8.70E−04 |
| A029400325 | 1.40E−03 | 9.70E−04 |
| A029400326 | 1.60E−03 | 1.10E−03 |
| A029400327 | 1.60E−03 | 1.10E−03 |
| A029400332 | 1.60E−03 | 1.10E−03 |
| A029400335 | 1.30E−03 | 8.00E−04 |
| A029400336 | 1.90E−03 | 1.40E−03 |
| A029400345 | 1.30E−03 | 7.60E−04 |
| A029400349 | 1.70E−03 | 1.20E−03 |
| A029400353 | 2.10E−03 | 1.50E−03 |
| A029400357 | 2.20E−03 | 1.20E−03 |
| A029400358 | 1.60E−03 | 1.00E−03 |
| A029400359 | 1.40E−03 | 9.80E−04 |
| A029400360 | 1.90E−03 | 1.10E−03 |
| A029400362 | 1.20E−03 | 8.40E−04 |
| A029400363 | 1.20E−03 | 8.20E−04 |
| A029400365 | 1.40E−03 | 9.10E−04 |
| A029400366 | 1.50E−03 | 1.00E−03 |
| A029400367 | 1.30E−03 | 8.30E−04 |
| A029400368 | 1.30E−03 | 8.90E−04 |
| A029400371 | 1.20E−03 | 8.40E−04 |
| A029400382 | 1.30E−03 | 8.30E−04 |
| A029400383 | 1.90E−03 | 1.40E−03 |
| A029400384 | 1.40E−03 | 9.00E−04 |
| A029400392 | 4.60E−03 | 3.70E−03 |
| A029400395 | 4.10E−03 | 3.40E−03 |
| A029400398 | 5.00E−03 | 4.30E−03 |
| A029400401 | 3.40E−03 | 4.10E−03 |
| A029400402 | 1.40E−03 | 9.30E−04 |
| A029400406 | 3.50E−03 | 3.10E−03 |
| A029400407 | 3.40E−03 | 2.70E−03 |
| A029400414 | 5.60E−03 | 5.90E−03 |
| A029400417 | 1.30E−03 | 9.20E−04 |
| A029400419 | 1.70E−03 | 1.20E−03 |
| A029400421 | 3.20E−03 | 2.70E−03 |
| A029400428 | 1.10E−02 | 1.00E−02 |
| A029400430 | 6.00E−03 | 6.80E−03 |
| A029400431 | 4.50E−03 | 5.10E−03 |
| A029400432 | 7.20E−03 | 1.10E−02 |
| A029400437 | 5.20E−03 | 4.70E−03 |
| A029400438 | 8.00E−03 | 9.10E−03 |
| A029400439 | 6.30E−03 | 8.30E−03 |
| A029400441 | 7.80E−03 | 6.70E−03 |
| A029400442 | 9.70E−03 | 1.20E−02 |
| A029400444 | 4.30E−03 | 3.50E−03 |
| A029400451 | 6.40E−03 | 7.90E−03 |
| A029400461 | 9.30E−03 | 1.60E−02 |
| A029400462 | 6.80E−03 | 1.00E−02 |
| A029400463 | 2.00E−03 | 1.90E−03 |
| A029400464 | 1.40E−03 | 8.80E−04 |
| A029400466 | 6.40E−03 | 5.30E−03 |
| A029400470 | 3.90E−03 | 5.40E−03 |
| A029400471 | 3.00E−03 | 4.20E−03 |
| A029400472 | 2.00E−03 | 3.30E−03 |
| A029400475 | 5.20E−03 | 8.40E−03 |
| A029400477 | 3.70E−03 | 9.40E−03 |
| A029400484 | 8.00E−03 | 7.20E−03 |
| A029400489 | 1.10E−02 | 2.20E−02 |
| A029400491 | nd | 2.50E−02 |
| A029400492 | 3.50E−03 | 3.80E−03 |
| A029400493 | 2.50E−03 | 2.20E−03 |
| A029400495 | 1.00E−03 | 5.00E−04 |
| A029400498 | 3.20E−03 | 9.30E−03 |
| A029400499 | 7.10E−03 | 6.20E−03 |
| A029400501 | 3.10E−03 | 2.00E−03 |
| A029400502 | 3.60E−03 | 3.40E−03 |
| A029400504 | 2.50E−03 | 1.70E−03 |
| A029400509 | 6.20E−03 | nd |
| A029400511 | 1.80E−03 | 3.60E−03 |
| A029400515 | 2.80E−03 | 3.80E−03 |
| A029400516 | 6.80E−03 | 5.50E−03 |
| A029400529 | 9.20E−03 | 7.60E−03 |
| A029400530 | 9.60E−03 | 1.70E−02 |
| A029400531 | 3.30E−03 | 9.20E−03 |
| A029400532 | 8.20E−03 | 1.10E−02 |
| A029400533 | 4.80E−03 | 1.10E−02 |
| A029400534 | 4.00E−03 | 9.50E−03 |
| A029400535 | 4.70E−03 | 1.70E−02 |
| A029400536 | 2.40E−03 | 5.50E−03 |
| A029400537 | 5.60E−03 | 1.40E−02 |
| A029400538 | 2.80E−03 | 3.30E−03 |
| A029400541 | 2.10E−03 | 3.10E−03 |
| A029400542 | 5.60E−03 | 9.50E−03 |
| A029400547 | 3.40E−03 | 4.10E−03 |
| A029400552 | 2.10E−03 | 2.20E−03 |
| A029400571 | 5.10E−03 | 6.20E−03 |
| A029400572 | 6.20E−03 | 5.20E−03 |
| A029400573 | 4.10E−03 | 3.20E−03 |
| A029400574 | 3.60E−03 | 2.50E−03 |
| A029400575 | 9.40E−03 | 8.80E−03 |
| A029400576 | 9.40E−03 | 7.70E−03 |
| A029400579 | 2.60E−03 | 1.70E−03 |
| A029400580 | 3.60E−03 | 2.80E−03 |
| A029400581 | 4.20E−03 | 3.10E−03 |
| A029400582 | 4.40E−03 | 3.40E−03 |
| A029400592 | 3.80E−03 | 2.80E−03 |
| A029400601 | 4.40E−03 | 4.10E−03 |
| A029400603 | 2.70E−03 | 2.60E−03 |
| A029400605 | 4.20E−03 | 7.50E−03 |
| A029400606 | 1.30E−02 | 2.90E−02 |
| A029400607 | 3.20E−03 | 6.20E−03 |
| A029400608 | 7.10E−03 | 1.40E−02 |
| A029400610 | 2.10E−03 | 2.60E−03 |
| A029400612 | 4.50E−03 | 1.10E−02 |
| A029400614 | 2.80E−03 | 3.30E−03 |
| A029400616 | 3.50E−03 | 2.90E−03 |
| A029400617 | 3.90E−03 | 7.00E−03 |
| A029400619 | 7.50E−03 | 7.70E−03 |
| A029400621 | 3.90E−03 | 4.40E−03 |
| A029400625 | 4.00E−03 | 8.60E−03 |
| A029400632 | 7.30E−03 | 1.50E−02 |
| A029400642 | 3.00E−03 | 2.00E−03 |
| A029400643 | 2.80E−03 | 1.90E−03 |
| A029400644 | 2.80E−03 | 2.10E−03 |
| A029400645 | 4.30E−03 | 3.80E−03 |
| A029400646 | 2.40E−03 | 1.60E−03 |
| A029400648 | 4.50E−03 | 3.30E−03 |
| A029400649 | 2.00E−03 | 1.10E−03 |
| A029400650 | 2.80E−03 | 1.80E−03 |
| A029400651 | 3.00E−03 | 2.30E−03 |
| A029400658 | 3.90E−03 | 2.90E−03 |
| A029400659 | 2.60E−03 | 1.60E−03 |
| A029400660 | 3.20E−03 | 2.30E−03 |
| A029400664 | 3.10E−03 | 2.20E−03 |
| A029400665 | 6.60E−03 | 5.70E−03 |
| A029400666 | 2.90E−03 | 2.00E−03 |
| A029400671 | 2.60E−03 | 1.80E−03 |
| A029400679 | 1.10E−03 | 6.80E−04 |
| A029400680 | 1.30E−03 | 8.10E−04 |
| A029400681 | 1.10E−03 | 6.10E−04 |
| A029400682 | 1.20E−03 | 8.30E−04 |
| A029400686 | 1.10E−03 | 8.00E−04 |
| A029400689 | 2.50E−03 | 2.00E−03 |
| A029400690 | 1.20E−03 | 6.70E−04 |
| A029400691 | 1.10E−03 | 7.40E−04 |
| A029400694 | 1.60E−03 | 1.20E−03 |
| A029400696 | 2.10E−03 | 1.30E−03 |
| A029400707 | 1.80E−03 | 1.10E−03 |
| A029400708 | 2.60E−03 | 1.80E−03 |
| A029400709 | 2.00E−03 | 1.50E−03 |
| A029400711 | 1.30E−03 | 8.80E−04 |
| A029400713 | 3.40E−03 | 2.40E−03 |
| A029400715 | 2.20E−03 | 1.60E−03 |
| A029400716 | 2.20E−03 | 1.50E−03 |
| A029400717 | 4.80E−03 | 4.10E−03 |
| A029400718 | 1.50E−03 | 8.70E−04 |
| A029400719 | 1.40E−03 | 7.10E−04 |
| A029400722 | 4.80E−03 | 3.50E−03 |
| A029400723 | 1.10E−03 | 5.40E−04 |
| A029400731 | 2.30E−03 | 1.50E−03 |
| A029400732 | 9.00E−03 | 9.80E−03 |
| A029400738 | 4.00E−03 | 3.50E−03 |
| A029400739 | 3.00E−03 | 2.20E−03 |
| A029400740 | 5.30E−03 | 4.60E−03 |
| A029400741 | 3.70E−03 | 2.90E−03 |
| A029400742 | 3.10E−03 | 2.20E−03 |
| A029400743 | 3.80E−03 | 3.30E−03 |
| A029400747 | 4.80E−03 | 3.80E−03 |
| A029400749 | 5.80E−03 | 6.50E−03 |
| A029400758 | 4.00E−03 | 3.20E−03 |
| A029400761 | 5.00E−03 | 5.10E−03 |
| A029400762 | 4.20E−03 | 3.40E−03 |
| A029400765 | 1.80E−03 | 1.20E−03 |
| A029400776 | 3.60E−03 | 4.50E−03 |
| A029400777 | 2.80E−03 | 2.20E−03 |
| A029400778 | 3.70E−03 | 2.90E−03 |
| A029400779 | 3.40E−03 | 3.40E−03 |
| A029400780 | 3.20E−03 | 2.60E−03 |
| A029400781 | 1.90E−03 | 2.80E−03 |
| A029400782 | 1.60E−03 | 2.00E−03 |
| A029400783 | 1.30E−03 | 1.20E−03 |
| A029400785 | 1.40E−03 | 1.30E−03 |
| A029400787 | 1.10E−03 | 1.10E−03 |
| A029400791 | 2.70E−03 | 2.50E−03 |
| A029400792 | 3.30E−03 | 4.20E−03 |
| A029400793 | 4.50E−03 | 5.80E−03 |
| A029400795 | 2.40E−03 | 2.10E−03 |
| A029400798 | 2.00E−03 | 2.00E−03 |
| A029400800 | 3.50E−03 | 3.90E−03 |
| A029400814 | 3.80E−03 | 3.10E−03 |
| A029400815 | 2.00E−03 | 1.30E−03 |
| A029400816 | 1.80E−03 | 1.30E−03 |
| A029400817 | 2.70E−03 | 1.90E−03 |
| A029400818 | 1.80E−03 | 1.20E−03 |
| A029400819 | 3.50E−03 | 2.80E−03 |
| A029400820 | 2.40E−03 | 1.70E−03 |
| A029400822 | 1.80E−03 | 1.30E−03 |
| A029400823 | 2.00E−03 | 1.40E−03 |
| A029400824 | 2.20E−03 | 1.60E−03 |
| A029400828 | 2.20E−03 | 1.60E−03 |
| A029400837 | 2.10E−03 | 1.50E−03 |
| A029400838 | 1.80E−03 | 1.40E−03 |
| A029400846 | 5.00E−01 | nd |
| A029400849 | 5.00E−01 | 1.50E−02 |
| A029400850 | 1.20E−02 | 9.80E−03 |
| A029400851 | 6.40E−03 | 3.90E−03 |
| A029400852 | 1.40E−02 | 1.00E−02 |
| A029400853 | 1.00E−02 | 7.40E−03 |
| A029400855 | 1.70E−02 | 1.40E−02 |
| A029400856 | 9.70E−03 | 8.00E−03 |
| A029400859 | 1.20E−02 | 1.00E−02 |
| A029400861 | 2.40E−03 | 2.00E−03 |
| A029400868 | 1.20E−02 | 1.40E−02 |
| A029400871 | 1.70E−03 | 1.20E−03 |
| A029400873 | 8.50E−03 | 8.70E−03 |
| A029400878 | 1.40E−02 | 1.10E−02 |
| A029400879 | 1.00E−02 | 7.70E−03 |
| A029400880 | 5.70E−03 | 3.80E−03 |
| A029400881 | 5.90E−03 | 4.00E−03 |
| A029400882 | 1.20E−02 | 8.00E−03 |
| A029400885 | 5.80E−03 | 4.20E−03 |
| A029400887 | 4.30E−03 | 3.00E−03 |
| A029400890 | 5.70E−03 | 3.80E−03 |
| A029400891 | 1.10E−02 | 7.50E−03 |
| A029400892 | 7.50E−03 | 5.30E−03 |
| A029400894 | 8.20E−03 | 6.60E−03 |
| A029400908 | 1.70E−03 | 1.10E−03 |
| A029400911 | 1.70E−03 | 1.10E−03 |
| A029400912 | 2.40E−03 | 1.60E−03 |
| A029400913 | 2.00E−03 | 1.30E−03 |
| A029400914 | 1.80E−03 | 1.20E−03 |
| A029400915 | 2.20E−03 | 1.40E−03 |
| A029400916 | 1.10E−03 | 7.50E−04 |
| A029400917 | 2.20E−03 | 1.40E−04 |
| A029400919 | 2.60E−03 | 1.60E−03 |
| A029400922 | 2.40E−03 | 1.50E−03 |
| A029400927 | 2.20E−03 | 1.40E−03 |
| A029400930 | 3.60E−03 | 2.50E−03 |
| A029400931 | 3.70E−03 | 2.50E−03 |
| A029400932 | 2.90E−03 | 1.90E−03 |
| A029400940 | 2.00E−03 | 1.40E−03 |
| A029400941 | 1.50E−03 | 9.70E−04 |
| A029400942 | 1.50E−03 | 1.00E−03 |
| A029400943 | 1.30E−03 | 8.90E−04 |
| A029400944 | 1.40E−03 | 9.60E−04 |
| A029400945 | 1.70E−03 | 1.10E−03 |
| A029400946 | 1.50E−03 | 1.00E−03 |
| A029400948 | 1.60E−03 | 1.00E−03 |
| A029400949 | 1.60E−03 | 1.10E−03 |
| A029400950 | 1.30E−03 | 9.10E−04 |
| A029400951 | 1.70E−03 | 1.10E−03 |
| A029400954 | 1.40E−03 | 9.60E−04 |
| A029400956 | 1.70E−03 | 1.10E−03 |
| A029400960 | 1.40E−03 | 9.40E−04 |
| A029400969 | 1.70E−03 | 1.10E−03 |
| A029400970 | 1.20E−03 | 1.00E−03 |
| A029400972 | 1.50E−03 | 9.50E−04 |
| A029400973 | 1.90E−03 | 1.30E−03 |
| A029400974 | 1.10E−03 | 9.90E−04 |
| A029400975 | 4.10E−03 | 3.10E−03 |
| A029400976 | 1.40E−03 | 9.30E−04 |
| A029400979 | 2.10E−03 | 1.40E−03 |
| A029400984 | 1.90E−03 | 1.30E−03 |
| A029400985 | 1.70E−03 | 1.10E−03 |
| A029400986 | 2.90E−03 | 2.00E−03 |
| A029400988 | 2.80E−03 | 2.20E−03 |
| A029400992 | 1.30E−03 | 1.20E−03 |
| A029401006 | 1.40E−03 | 8.50E−04 |
| A029401007 | 1.40E−03 | 9.40E−04 |
| A029401009 | 1.20E−03 | 1.00E−03 |
| A029401010 | 1.40E−03 | 9.40E−04 |
| A029401012 | 1.50E−03 | 1.00E−03 |
| A029401013 | 1.50E−03 | 1.00E−03 |
| A029401016 | 1.40E−03 | 9.30E−04 |
| A029401017 | 1.10E−03 | 9.50E−04 |
| A029401018 | 1.40E−03 | 9.30E−04 |
| A029401019 | 1.20E−03 | 9.20E−04 |
| A029401021 | 1.50E−03 | 1.10E−03 |
| A029401028 | 1.80E−03 | 1.20E−03 |
| A029401036 | 1.70E−03 | 1.10E−03 |
| A029401037 | 1.80E−03 | 1.20E−03 |
| A029401041 | 3.30E−03 | 2.00E−03 |
| A029401042 | 2.30E−02 | 1.10E−02 |
| A029401045 | 2.70E−02 | 2.10E−02 |
| A029401047 | 1.60E−03 | 1.40E−03 |
| A029401051 | 4.90E−03 | 4.60E−03 |
| A029401052 | 2.10E−03 | 1.50E−03 |
| A029401053 | 1.60E−03 | 1.20E−03 |
| A029401054 | 9.10E−04 | 8.00E−04 |
| A029401057 | 2.10E−03 | 1.50E−03 |
| A029401060 | 2.40E−03 | 2.10E−03 |
| A029401063 | 5.20E−03 | 3.90E−03 |
| A029401064 | 1.80E−03 | 1.40E−03 |
| A029401065 | 1.50E−03 | 1.30E−03 |
| A029401068 | 3.80E−03 | 2.90E−03 |
| A029401069 | 3.80E−03 | 2.80E−03 |
| A029401070 | 5.50E−03 | 4.20E−03 |
| A029401071 | 7.30E−03 | 5.20E−03 |
| A029401074 | 3.70E−03 | 3.00E−03 |
| A029401075 | 1.10E−03 | 9.60E−04 |
| A029401076 | 2.70E−03 | 2.10E−03 |
| A029401077 | 1.80E−03 | 1.20E−03 |
| A029401080 | 2.30E−03 | 1.70E−03 |
| A029401081 | 3.50E−03 | 2.70E−03 |
| A029401082 | 2.10E−03 | 1.70E−03 |
| A029401084 | 3.70E−03 | 2.80E−03 |
| A029401089 | 1.60E−03 | 1.20E−03 |
| A029401092 | 2.50E−03 | 2.00E−03 |
| A029401098 | 5.80E−03 | 6.20E−03 |
| A029401099 | 1.30E−02 | 1.00E−02 |
| A029401100 | 1.80E−03 | 1.70E−03 |
| A029401101 | 8.80E−03 | 5.90E−03 |
| A029401103 | 2.60E−03 | 2.70E−03 |
| A029401104 | 3.80E−03 | 4.40E−03 |
| A029401105 | 1.40E−03 | 1.30E−03 |
| A029401107 | 2.30E−03 | 2.00E−03 |
| A029401108 | 2.40E−03 | 2.30E−03 |
| A029401109 | 1.90E−03 | 1.90E−03 |
| A029401110 | 1.70E−03 | 1.70E−03 |
| A029401118 | 3.70E−03 | 4.10E−03 |
| A029401121 | 1.50E−03 | 1.50E−03 |
| A029401129 | 2.50E−03 | 2.50E−03 |
| A029401130 | 3.40E−03 | 2.50E−03 |
| A029401131 | 1.70E−03 | 1.20E−03 |
| A029401132 | 2.80E−03 | 2.00E−03 |
| A029401133 | 1.80E−03 | 1.30E−03 |
| A029401134 | 4.10E−03 | 3.00E−03 |
| A029401135 | 1.60E−03 | 1.20E−03 |
| A029401136 | 2.00E−03 | 1.40E−03 |
| A029401137 | 1.70E−03 | 1.30E−03 |
| A029401138 | 1.90E−03 | 1.30E−03 |
| A029401141 | 2.90E−03 | 2.30E−03 |
| A029401143 | 1.60E−03 | 1.50E−03 |
| A029401145 | 1.80E−03 | 1.40E−03 |
| A029401146 | 1.60E−03 | 1.10E−03 |
| A029401150 | 1.60E−03 | 1.10E−03 |
| A029401152 | 3.60E−03 | 2.80E−03 |
| A029401165 | 3.70E−03 | 4.20E−03 |
| A029401166 | 1.50E−03 | 1.20E−03 |
| A029401167 | 3.70E−03 | 2.70E−03 |
| A029401168 | 2.10E−03 | 9.90E−04 |
| A029401169 | 2.20E−03 | 1.50E−03 |
| A029401170 | 1.90E−03 | 8.70E−04 |
| A029401172 | 2.50E−03 | 1.40E−03 |
| A029401173 | 3.30E−03 | 2.80E−03 |
| A029401174 | 2.70E−03 | 1.80E−03 |
| A029401175 | 3.00E−03 | 2.40E−03 |
| A029401177 | 2.20E−03 | 1.20E−03 |
| A029401178 | 2.40E−03 | 2.30E−03 |
| A029401179 | 4.10E−03 | 3.60E−03 |
| A029401187 | 2.40E−03 | 1.70E−03 |
| A029401190 | 4.20E−03 | 3.90E−03 |
| A029401202 | 1.40E−03 | 1.20E−03 |
| A029401203 | 1.40E−03 | 1.40E−03 |
| A029401204 | 1.30E−03 | 1.20E−03 |
| A029401205 | 1.40E−03 | 1.40E−03 |
| A029401206 | 1.50E−03 | 1.40E−03 |
| A029401209 | 1.30E−03 | 1.20E−03 |
| A029401210 | 2.80E−03 | 2.80E−03 |
| A029401211 | 1.80E−03 | 1.60E−03 |
| A029401212 | 1.50E−03 | 1.70E−03 |
| A029401213 | 1.40E−03 | 1.30E−03 |
| A029401214 | 1.10E−03 | 8.30E−04 |
| A029401215 | 1.30E−03 | 1.30E−03 |
| A029401219 | 1.10E−03 | 8.70E−04 |
| A029401222 | 1.40E−03 | 1.40E−03 |
| A029401224 | 1.80E−03 | 1.80E−03 |
| A029401240 | 2.20E−03 | 2.40E−03 |
| A029401241 | 1.40E−02 | 1.60E−02 |
| A029401242 | 1.40E−02 | 8.30E−03 |
| A029401243 | 1.20E−02 | 1.20E−02 |
| A029401244 | 4.50E−03 | 2.50E−03 |
| A029401247 | 1.20E−02 | 1.30E−02 |
| A029401248 | 6.60E−03 | 8.90E−03 |
| A029401249 | 1.20E−02 | 1.20E−02 |
| A029401250 | 1.00E−02 | 1.20E−02 |
| A029401251 | 6.30E−03 | 9.00E−03 |
| A029401252 | 1.10E−02 | 9.30E−03 |
| A029401254 | 2.40E−03 | 2.80E−03 |
| A029401257 | 4.70E−03 | 6.80E−03 |
| A029401259 | 8.80E−03 | 1.00E−02 |
| A029401267 | 4.50E−03 | 5.30E−03 |
| A029401273 | 9.80E−03 | 6.70E−03 |
| A029401275 | 2.50E−03 | 2.40E−03 |
| A029401278 | 7.10E−03 | 9.20E−03 |
| A029401280 | 1.90E−03 | 3.00E−03 |
| A029401281 | 5.70E−03 | 1.50E−02 |
| A029401282 | 2.00E−03 | 2.40E−03 |
| A029401283 | 8.90E−03 | 1.40E−02 |
| A029401284 | 8.70E−03 | 1.40E−02 |
| A029401286 | nd | 9.40E−03 |
| A029401289 | 2.90E−03 | 3.40E−03 |
| A029401290 | 1.40E−03 | 3.20E−04 |
| A029401292 | 3.50E−03 | 3.80E−03 |
| A029401296 | 3.00E−03 | 8.30E−04 |
| A029401298 | 7.00E−03 | 1.60E−02 |
| A029401303 | 2.00E−03 | 1.40E−03 |
| A029401308 | 3.00E−03 | 1.70E−03 |
| A029401309 | 3.80E−03 | 2.90E−03 |
| A029401310 | 3.00E−03 | 2.40E−03 |
| A029401311 | 3.80E−03 | 3.20E−03 |
| A029401312 | 2.80E−03 | 2.00E−03 |
| A029401313 | 2.70E−03 | 2.00E−03 |
| A029401314 | 3.30E−03 | 2.90E−03 |
| A029401315 | 7.70E−03 | 7.70E−03 |
| A029401316 | 2.60E−03 | 1.90E−03 |
| A029401320 | 4.20E−03 | 3.30E−03 |
| A029401327 | 2.90E−03 | 2.30E−03 |
| A029401330 | 4.90E−03 | 5.10E−03 |
| A029401336 | 1.40E−03 | 9.80E−04 |
| A029401337 | 3.00E−03 | 2.10E−03 |
| A029401341 | 1.60E−03 | 1.10E−03 |
| A029401342 | 2.00E−03 | 1.40E−03 |
| A029401343 | 1.70E−03 | 1.00E−03 |
| A029401344 | 1.50E−03 | 1.00E−03 |
| A029401345 | 2.10E−03 | 1.40E−03 |
| A029401346 | 1.90E−03 | 1.40E−03 |
| A029401347 | 1.70E−03 | 1.20E−03 |
| A029401348 | 1.90E−03 | 1.40E−03 |
| A029401350 | 2.00E−03 | 1.50E−03 |
| A029401352 | 1.30E−03 | 9.20E−04 |
| A029401354 | 2.20E−03 | 1.70E−03 |
| A029401357 | 1.70E−03 | 1.40E−03 |
| A029401367 | 1.60E−03 | 1.10E−03 |
| A029401368 | 2.00E−03 | 1.50E−03 |
| A029401372 | 1.00E−03 | 8.60E−04 |
| A029401374 | 3.30E−03 | 2.60E−03 |
| A029401377 | 4.00E−03 | 2.60E−03 |
| A029401378 | 7.40E−03 | 5.10E−03 |
| A029401379 | 1.90E−03 | 1.40E−03 |
| A029401380 | 1.40E−02 | 1.30E−02 |
| A029401381 | 9.50E−03 | 7.50E−03 |
| A029401382 | 9.30E−03 | 8.30E−03 |
| A029401383 | 1.80E−03 | 1.40E−03 |
| A029401385 | 3.00E−03 | 2.10E−03 |
| A029401386 | 2.10E−03 | 1.60E−03 |
| A029401391 | 1.80E−03 | 1.40E−03 |
| A029401392 | 1.70E−03 | 1.50E−03 |
| A029401393 | 2.30E−03 | 1.70E−03 |
| A029401398 | 1.70E−03 | 1.30E−03 |
| A029401408 | 1.70E−03 | 1.40E−03 |
| A029401411 | 2.40E−03 | 2.00E−03 |
| A029401415 | 1.30E−03 | 9.80E−04 |
| A029401416 | 1.10E−03 | 9.00E−04 |
| A029401417 | 1.30E−03 | 8.70E−04 |
| A029401418 | 1.30E−03 | 9.80E−04 |
| A029401419 | 1.10E−03 | 9.40E−04 |
| A029401420 | 1.70E−03 | 1.50E−03 |
| A029401422 | 2.10E−03 | 1.50E−03 |
| A029401423 | 1.10E−03 | 8.80E−04 |
| A029401424 | 1.40E−03 | 1.00E−03 |
| A029401428 | 1.10E−03 | 9.50E−04 |
| A029401430 | 1.60E−03 | 1.20E−03 |
| A029401437 | 1.10E−03 | 1.10E−03 |
| A029401440 | 1.80E−03 | 1.20E−03 |
| A029404430 | 2.00E−03 | 1.60E−03 |
| A029404431 | 2.00E−03 | 1.70E−03 |
| A029404432 | 1.70E−03 | 1.50E−03 |
| A029404433 | 1.50E−03 | 1.40E−03 |
| A029404434 | 1.60E−03 | 1.40E−03 |
| A029404435 | 1.40E−03 | 1.30E−03 |
| A029404436 | 1.30E−03 | 1.10E−03 |
| A029404437 | 1.30E−03 | 1.20E−03 |
| A029404438 | 1.90E−03 | 2.20E−03 |
| A029404439 | 1.90E−03 | 2.00E−03 |
| A029404440 | 3.70E−02 | 1.30E−02 |
| A029404441 | 5.60E−02 | 1.70E−02 |
| A029404442 | 2.30E−03 | 2.60E−03 |
| A029404443 | 1.90E−03 | 1.90E−03 |
| A029404444 | 2.40E−03 | 3.80E−02 |
| A029404445 | 2.60E−03 | 2.90E−03 |
| A029404446 | 1.90E−03 | 1.50E−03 |
| A029404447 | 2.20E−03 | 1.60E−03 |
| A029404448 | 1.70E−03 | 1.40E−03 |
| A029404449 | 5.20E−02 | 1.20E−03 |
| A029404450 | 1.70E−03 | 1.30E−03 |
| A029404451 | 1.40E−03 | 1.30E−03 |
| A029404452 | 1.90E−02 | 1.30E−03 |
| A029404453 | 1.20E−03 | 1.10E−03 |
| A029404454 | 2.20E−03 | 2.70E−03 |
| A029404455 | 1.60E−03 | 1.90E−03 |
| A029404456 | 9.20E−02 | 1.10E−02 |
| A029404457 | 3.70E−03 | 1.40E−02 |
| A029404458 | 2.60E−03 | 2.80E−03 |
| A029404459 | 2.40E−03 | 2.30E−03 |
| A029404460 | 3.90E−03 | 3.80E−02 |
| A029404461 | 4.10E−03 | 2.40E−02 |
| A029404462 | 1.90E−03 | 1.40E−03 |
| A029404463 | 1.50E−03 | 1.20E−03 |
| A029404464 | 1.50E−03 | 1.40E−03 |
| A029404465 | 1.60E−03 | 1.40E−03 |
| A029404466 | 1.70E−03 | 1.50E−03 |
| A029404467 | 1.60E−03 | 1.40E−03 |
| A029404468 | 1.50E−03 | 1.30E−03 |
| A029404469 | 1.60E−03 | 1.30E−03 |
| A029404470 | 4.50E−03 | 4.80E−03 |
| A029404471 | 4.30E−03 | 4.70E−03 |
| A029404472 | 8.10E−03 | 4.00E−03 |
| A029404474 | 3.40E−03 | 3.80E−03 |
| A029404475 | 3.80E−03 | 4.10E−03 |
| A029404476 | 3.00E−02 | 7.20E−03 |
| A029404477 | 3.70E−03 | 8.50E−03 |
| A029404478 | 1.60E−03 | 1.10E−03 |
| A029404479 | 1.60E−03 | 1.10E−03 |
| A029404480 | 1.80E−03 | 1.60E−03 |
| A029404481 | 1.70E−03 | 1.40E−03 |
| A029404482 | 1.70E−03 | 1.30E−03 |
| A029404483 | 2.00E−03 | 1.40E−03 |
| A029404484 | 1.70E−03 | 1.50E−03 |
| A029404485 | 1.50E−03 | 1.20E−03 |
| A029404486 | 3.20E−03 | 3.40E−03 |
| A029404487 | 4.00E−03 | 4.40E−03 |
| A029404488 | 2.10E−02 | 6.40E−03 |
| A029404489 | 3.90E−03 | 7.10E−03 |
| A029404490 | 3.90E−03 | 4.00E−03 |
| A029404491 | 3.20E−03 | 3.40E−03 |
| A029404492 | 3.90E−03 | 1.00E−02 |
| A029404493 | 4.10E−03 | 1.60E−02 |
| A029404494 | 2.20E−03 | 2.00E−03 |
| A029404495 | 2.30E−03 | 1.10E−03 |
| A029404496 | 2.00E−03 | 1.50E−03 |
| A029404497 | 1.80E−03 | 1.50E−03 |
| A029404498 | 2.10E−03 | 1.40E−03 |
| A029404499 | 1.90E−03 | 1.50E−03 |
| A029404500 | 1.50E−03 | 1.20E−03 |
| A029404501 | 1.70E−03 | 1.40E−03 |
| A029404502 | 3.50E−03 | 3.90E−03 |
| A029404503 | 3.80E−03 | 4.40E−03 |
| A029404504 | 1.30E−02 | 6.10E−03 |
| A029404505 | 4.80E−02 | 8.20E−03 |
| A029404506 | 3.90E−03 | 4.70E−03 |
| A029404507 | 3.70E−03 | 4.80E−03 |
| A029404508 | 3.30E−03 | 1.50E−02 |
| A029404509 | 4.00E−03 | 1.60E−02 |
| A029404510 | 2.50E−03 | 1.40E−03 |
| A029404511 | 2.40E−03 | 1.50E−03 |
| A029404512 | 1.90E−03 | 1.80E−03 |
| A029404513 | 1.90E−03 | 1.80E−03 |
| A029404514 | 4.60E−03 | 2.60E−03 |
| A029404515 | 2.10E−03 | 2.10E−03 |
| A029404516 | 3.20E−03 | 2.30E−03 |
| A029404517 | 1.70E−03 | 1.80E−03 |
| A029404518 | 2.40E−03 | 3.10E−03 |
| A029404519 | 2.10E−03 | 2.90E−03 |
| A029404520 | 2.10E−03 | 2.50E−03 |
| A029404521 | 2.30E−03 | 2.60E−03 |
| A029404522 | 2.60E−03 | 3.40E−03 |
| A029404523 | 2.20E−03 | 2.90E−03 |
| A029404524 | 1.80E−03 | 2.10E−03 |
| A029404525 | 2.10E−03 | 2.60E−03 |
| A029404526 | 3.00E−03 | 2.00E−03 |
| A029404527 | 2.80E−03 | 2.10E−03 |
| A029404528 | 1.50E−03 | 1.50E−03 |
| A029404529 | 2.00E−03 | 2.60E−03 |
| A029404530 | 2.10E−03 | 1.60E−03 |
| A029404531 | 2.00E−03 | 1.50E−03 |
| A029404532 | 1.50E−03 | 1.60E−03 |
| A029404533 | 5.20E−02 | 2.00E−03 |
| A029404534 | 1.90E−03 | 1.50E−03 |
| A029404535 | 1.80E−03 | 1.40E−03 |
| A029404536 | 1.80E−03 | 2.60E−03 |
| A029404537 | 1.90E−03 | 1.50E−03 |
| A029404538 | 1.90E−03 | 1.40E−03 |
| A029404539 | 1.90E−03 | 1.40E−03 |
| A029404540 | 1.70E−03 | 1.70E−03 |
| A029404541 | 1.90E−03 | 1.50E−03 |
| A029404542 | 1.70E−03 | 1.20E−03 |
| A029404543 | 1.70E−03 | 8.50E−04 |
| A029404544 | 2.70E−02 | 1.60E−03 |
| A029404545 | 1.80E−03 | 1.60E−03 |
| A029404546 | 2.20E−03 | 1.50E−03 |
| A029404547 | 2.00E−03 | 1.40E−03 |
| A029404548 | 1.70E−03 | 1.80E−03 |
| A029404549 | 1.70E−03 | 1.70E−03 |
| A029404550 | 1.90E−03 | 1.20E−03 |
| A029404551 | 1.80E−03 | 1.30E−03 |
| A029404552 | 1.80E−03 | 2.10E−03 |
| A029404553 | 2.00E−03 | 2.30E−03 |
| A029404554 | 1.90E−03 | 1.60E−03 |
| A029404555 | 1.80E−03 | 1.40E−03 |
| A029404556 | 1.80E−03 | 1.90E−03 |
| A029404557 | 1.90E−03 | 2.10E−03 |
| A029404558 | 1.70E−03 | 1.10E−03 |
| A029404559 | 1.80E−03 | 1.30E−03 |
| A029404560 | 5.20E−03 | 1.80E−03 |
| A029404561 | 1.80E−03 | 2.00E−03 |
| A029404562 | 2.00E−03 | 1.60E−03 |
| A029404563 | 1.90E−03 | 2.10E−03 |
| A029404564 | 3.20E−03 | 2.10E−03 |
| A029404565 | 1.50E−03 | 1.60E−03 |
| A029404566 | 1.50E−03 | 1.10E−03 |
| A029404567 | 1.40E−03 | 9.30E−04 |
| A029404568 | 1.50E−03 | 1.20E−03 |
| A029404569 | 1.50E−03 | 1.00E−03 |
| A029404570 | 1.50E−03 | 1.00E−03 |
| A029404571 | 1.50E−03 | 1.00E−03 |
| A029404572 | 1.50E−03 | 1.10E−03 |
| A029404573 | 1.30E−03 | 9.80E−04 |
| A029404574 | 7.80E−04 | nd |
| A029404575 | 1.20E−03 | nd |
| A029404576 | 1.80E−03 | 1.90E−03 |
| A029404577 | 1.90E−03 | 1.50E−03 |
| A029404578 | 2.00E−03 | 1.50E−03 |
| A029404579 | 1.70E−03 | 1.70E−03 |
| A029404580 | 1.60E−03 | 1.70E−03 |
| A029404581 | 1.70E−03 | 1.30E−03 |
| A029404582 | 1.80E−03 | 1.50E−03 |
| A029404583 | 1.90E−03 | 2.00E−03 |
| A029404584 | 1.70E−03 | 1.30E−03 |
| A029404585 | 2.50E−03 | 2.00E−03 |
| A029404586 | 2.20E−03 | 1.80E−03 |
| A029404587 | 2.10E−03 | 1.80E−03 |
| A029404588 | 7.80E−03 | 2.00E−03 |
| A029404589 | 2.50E−03 | 1.90E−03 |
| A029404590 | 2.00E−03 | 1.70E−03 |
| A029404591 | 2.00E−03 | 1.70E−03 |
| A029404592 | 2.90E−03 | 2.00E−03 |
| A029404593 | 3.10E−03 | 2.10E−03 |
| A029404594 | 2.40E−03 | 1.90E−03 |
| A029404595 | 2.40E−03 | 1.80E−03 |
| A029404596 | 2.70E−03 | 2.10E−03 |
| A029404597 | 2.70E−03 | 1.90E−03 |
| A029404598 | 2.30E−03 | 1.80E−03 |
| A029404599 | 2.60E−03 | 1.90E−03 |
| A029404600 | 5.60E−02 | 2.20E−03 |
| A029404601 | 2.70E−03 | 2.30E−03 |
| A029404603 | 2.20E−03 | 2.00E−03 |
| A029404604 | 1.80E−03 | 1.50E−03 |
| A029404605 | 2.10E−03 | 1.70E−03 |
| A029404606 | 1.60E−03 | 1.40E−03 |
| A029404607 | 1.70E−03 | 1.40E−03 |
| A029404621 | 2.00E−03 | 1.80E−03 |
| A029404622 | 2.50E−03 | 2.20E−03 |
| A029404623 | 2.10E−03 | 1.80E−03 |
| A029404624 | 2.00E−03 | 1.70E−03 |
| A029404625 | 2.60E−03 | 2.00E−03 |

nd: not determined

TABLE B-16
Off-rate values for the sequence optimized variants
of ISVD A0294037F03 measured at 37° C.

| | cPD-1 | hPD-1 |
|---|---|---|
| | koff | koff |
| ISVD ID | (1/s) | (1/s) |
| A0294037F03 | 4.65E−04 | 3.38E−04 |
| A029400074 | 2.65E−04 | 2.06E−04 |
| A029400104 | 9.96E−04 | 5.97E−04 |
| A029400105 | 8.38E−04 | 4.83E−04 |
| A029400106 | 8.82E−04 | 4.94E−04 |
| A029400107 | 3.25E−04 | 2.02E−04 |
| A029400108 | 3.41E−04 | 2.34E−04 |
| A029400109 | 3.11E−04 | 2.07E−04 |
| A029400110 | 3.30E−04 | 2.30E−04 |
| A029400111 | 8.36E−04 | 4.79E−04 |
| A029400161 | 3.21E−04 | 2.12E−04 |
| A029400169 | 1.14E−03 | 6.20E−04 |
| A029400170 | 3.80E−04 | 2.30E−04 |
| A029400171 | 2.53E−04 | 1.73E−04 |
| A029400172 | 9.45E−04 | 5.12E−04 |
| A029400173 | 8.98E−04 | 4.54E−04 |
| A029400174 | 1.05E−03 | 5.80E−04 |
| A029400175 | 3.74E−04 | 2.52E−04 |
| A029400176 | 3.04E−04 | 1.81E−04 |
| A029400177 | 2.67E−04 | 1.60E−04 |
| A029400178 | 3.45E−04 | 2.59E−04 |
| A029403750 | 5.6E−03 | 2.4E−03 |
| A029403751 | 3.6E−03 | 1.6E−03 |
| A029403752 | 4.2E−03 | 1.4E−03 |
| A029403753 | 3.2E−03 | 1.3E−03 |
| A029403754 | 4.3E−03 | 1.7E−03 |
| A029403755 | 4.6E−03 | 1.7E−03 |
| A029403756 | 3.7E−03 | 1.3E−03 |
| A029403757 | 3.3E−03 | 1.2E−03 |
| A029403758 | 4.7E−03 | 1.5E−03 |
| A029403759 | 3.1E−03 | 1.2E−03 |
| A029403760 | 3.1E−03 | 1.2E−03 |
| A029403761 | 3.2E−03 | 1.2E−03 |
| A029403762 | 3.3E−03 | 1.2E−03 |
| A029403763 | 3.3E−03 | 1.2E−03 |
| A029403764 | 3.1E−03 | 1.1E−03 |
| A029404678 | 8.53E−03 | 7.11E−03 |
| A029404679 | 9.90E−03 | 8.02E−03 |
| A029404682 | nd | 1.31E−02 |
| A029404683 | 1.53E−02 | 1.43E−02 |
| A029404684 | 3.96E−03 | 2.44E−03 |
| A029404685 | 3.89E−03 | 2.30E−03 |

nd: not determined

The sequence optimized ISVDs kept a good off-rate values (kd) in the range of about 1E-03 to 1E-04.

### 5.19.3 Biophysical Characterization of the Sequence Optimized ISVDs

The biophysicial characteristics of the sequence optimized ISVDs of ISVD A0294037F03 were tested in thermal shift assay (Tm) and a temperature of onset of aggregation assay (Tagg).

The thermal shift assay and Tagg assay were performed as described in example 6.5.1 and 6.5.4 respectively. The Tm and Tagg values obtained for the sequence optimized variants are shown in Table B-17.

TABLE B-17
Biophysical properties of the sequence optimized
variants of ISVD A0294037F03

| | Tm | Tagg |
|---|---|---|
| ISVD ID | (° C.) | (° C.) |
| A0294037F03 | 64 | nd |
| A029400074 | 66.6 | nd |
| A029400104 | nd | 61.5 |
| A029400106 | nd | 61.5 |
| A029400107 | 65.6 | nd |
| A029400108 | 67.7 | 61.5 |
| A029400109 | nd | 61.5 |
| A029400110 | 66.3 | 62.7 |
| A029400111 | nd | 61.2 |
| A029400161 | 68 | 62.4 |
| A029400171 | 57.9 | 55.7 |
| A029400174 | 70.3 | 65.3 |
| A029400175 | 71.7 | 67.4 |
| A029400178 | 56.9 | 56.8 |
| A029403741 | 79 | nd |
| A029403742 | 82 | nd |
| A029403743 | 73 | nd |
| A029403744 | 76 | nd |
| A029404010 | 73.0 | 64.2 |
| A029404011 | 72.0 | 63.6 |
| A029404015 | 77.8 | 69 |
| A029404016 | 82.7 | 76.4 |
| A029404017 | 78.3 | 69.9 |
| A029404030 | 81.7 | 75.4 |
| A029404032 | 79.3 | 71 |
| A029404034 | 77.8 | 70.9 |
| A029404473 | 76 | nd |
| A029404680 | 63.2 | 55.7 |
| A029404681 | 63.7 | 55.2 |
| A029404684 | 64.2 | nd |
| A029404685 | 64.2 | nd |
| A029404782 | 63 | nd |
| A029404797 | 70 | nd |

nd: not determined

### 5.19.4 Characterization of the Sequence Optimized ISVDs in Competition with PD-L1

The sequence optimized variants of ISVD A0294037F03 were tested for by FACS competition assay using hPD-L1-Fc Biotinylated (Sino Biological) as competitor to CHO cells expressing human or cyno PD-1. The binding FACS competition assay was carried out as described in Example 6.1.3. Results are shown in Tables B-18.

TABLE B-18
Results of FACS competition assay with the sequence optimized
variants of ISVD A0294037F03 using hPD-L1-Fc Biotinylated
(Sino Biological) as competitor to CHO cells expressing human
or cyno PD-1

| | IC50 |
|---|---|
| ISVD ID | (M) |
| A0294037F03 | 5.94E−09 |
| A029400250 | 3.78E−09 |
| A029400319 | 3.24E−09 |
| A029400320 | 3.82E−09 |
| A029400321 | 4.40E−09 |
| A029400323 | 2.87E−09 |
| A029400324 | 3.36E−09 |
| A029400325 | 2.89E−09 |
| A029400335 | 4.22E−09 |
| A029400345 | 3.30E−09 |
| A029400362 | 3.35E−09 |
| A029400363 | 2.77E−09 |
| A029400366 | 3.81E−09 |
| A029400367 | 5.09E−09 |
| A029400368 | 3.01E−09 |
| A029400371 | 3.40E−09 |
| A029400382 | 3.57E−09 |
| A029400384 | 4.48E−09 |
| A029400402 | 2.74E−09 |
| A029400417 | 4.55E−09 |
| A029400464 | 6.82E−05 |
| A029400495 | 1.03E−08 |
| A029400539 | 1.57E−02 |
| A029400679 | 2.99E−09 |
| A029400680 | 3.40E−09 |
| A029400681 | 3.02E−09 |
| A029400682 | 3.26E−09 |
| A029400686 | 2.75E−09 |
| A029400690 | 3.29E−09 |
| A029400691 | 4.50E−09 |
| A029400711 | 2.91E−09 |
| A029400718 | 3.21E−09 |
| A029400719 | 2.86E−09 |
| A029400723 | 1.87E−09 |
| A029400783 | 4.99E−10 |
| A029400787 | 7.72E−08 |
| A029400847 | 4.89E−08 |
| A029400848 | 3.23E−04 |
| A029400916 | 2.93E−09 |
| A029400941 | 3.22E−09 |
| A029400942 | 3.56E−09 |
| A029400943 | 5.23E−09 |
| A029400944 | 2.45E−09 |
| A029400946 | 3.13E−09 |
| A029400954 | 2.81E−09 |
| A029400960 | 3.07E−09 |
| A029400972 | 3.96E−09 |
| A029400976 | 5.50E−09 |
| A029401006 | 1.58E−09 |
| A029401007 | 4.31E−09 |
| A029401009 | 2.42E−09 |
| A029401010 | 4.41E−09 |
| A029401013 | 3.21E−09 |
| A029401016 | 2.90E−09 |
| A029401017 | 1.63E−12 |
| A029401018 | 2.64E−09 |
| A029401019 | 3.66E−09 |
| A029401043 | 8.32E−10 |
| A029401046 | 3.26E−09 |
| A029401075 | 2.79E−11 |
| A029401202 | 3.30E−09 |
| A029401204 | 3.85E−10 |
| A029401209 | 1.93E−09 |
| A029401214 | 2.68E−09 |
| A029401215 | 2.62E−09 |
| A029401219 | 3.54E−09 |
| A029401290 | 5.32E−10 |
| A029401336 | 3.23E−09 |
| A029401352 | 2.91E−09 |
| A029401417 | 2.71E−09 |
| A029401419 | 3.03E−09 |
| A029401423 | 3.00E−09 |
| A029401437 | 4.25E−09 |

### 5.19.5 PD-1/PD-L1 Blocking Assay

The sequence optimized variants of ISVD A0294037F03 were tested on a functional NFAT reporter assay, as described above (see 6.1.3). Results of the PD-1/PD-L1 blocking assay are shown in Table B-19.

TABLE B-19
Results of PD-1/PD-L1 blocking assay with
optimized variants of ISVD A0294037F03

| | Potency | % |
|---|---|---|
| ISVD ID | (M) | Inhibition |
| A0294037F03 | 4.62E−9 | nd |
| A029404015 | 1.67E−09 | 109.67 |
| A029404473 | 1.61E−09 | 112.81 |
| A029404680 | 1.472E−05 | nd |
| A029404681 | 1.583E−05 | nd |
| A029405065 | 1.46E−09 | 98.79 |
| A029405164 | 8.79E−10 | 103.91 |
| A029405166 | 1.93E−09 | 90.56 |
| A029405167 | 2.81E−09 | 97.73 |
| A029405168 | 2.92E−09 | 101.34 |
| A029405169 | 5.43E−09 | 103.46 |
| A029405170 | 1.10E−09 | 116.83 |
| A029405171 | 2.04E−09 | 115.70 |
| A029405174 | 6.45E−09 | 125.95 |
| A029405175 | 5.25E−10 | 96.27 |
| A029405177 | 3.67E−08 | 113.67 |
| A029405178 | 3.465E−09 | 103.27 |
| A029405179 | 5.68E−09 | 99.86 |
| A029405180 | 4.73E−09 | 106.13 |
| A029405182 | 2.97E−09 | 104.37 |
| A029405183 | 1.84E−09 | 103.54 |
| A029405184 | 3.52E−09 | 108.41 |
| A029405185 | 4.87E−09 | 111.49 |

nd: not determined

### 5.19.6 Affinity Determination of the Sequence Optimized Variants

Affinity determination of sequence optimized variants of ISVD A0294037F03 was performed as describe in 6.4, but at 37° C. Results are summarized in Table B-20.

TABLE B-20
Affinity determination of the sequence optimization variants of ISVD A0294037F03

| | Human PD-1 | | | Cyno PD-1 | | |
|---|---|---|---|---|---|---|
| ISVD ID | Kon (1/Ms) | Koff (1/s) | KD (M) | Kon (1/Ms) | Koff (1/s) | KD (M) |
| A0294037F03 | 3.36E+05 | 1.23E−03 | 3.67E−09 | 2.29E+05 | 1.96E−03 | 8.53E−09 |
| A029400074 | 3.42E+05 | 1.15E−03 | 3.36E−09 | 2.34E+05 | 1.65E−03 | 7.06E−09 |
| A029403741 | 3.66E+05 | 3.26E−03 | 8.91E−09 | 2.09E+05 | 4.74E−03 | 2.27E−08 |
| A029403742 | 3.83E+05 | 4.09E−03 | 1.07E−08 | 2.14E+05 | 5.91E−03 | 2.77E−08 |
| A029403743 | 4.63E+05 | 1.12E−03 | 2.43E−09 | 3.19E+05 | 1.59E−03 | 4.99E−09 |
| A029403744 | 5.34E+05 | 1.29E−03 | 2.41E−09 | 3.72E+05 | 1.81E−03 | 4.87E−09 |

### 5.19.7 Functional Testing of the Sequence Optimized Variants in Tetanus-Toxoid Antigen-Specific Co-Culture Assay (TT Assay)

The sequence optimized variants of ISVD A0294037F03 were tested for primary T cell activation (monitored via IFNγ production) in an autologous Tetanus Toxoid recall assay as described in Example 6.7. Results are summarized in Table B-21A.

TABLE B-21A
EC50 values of the sequence optimization variants
of ISVD A0294037F03 in the TT assay

| | donor 1 | donor 2 |
|---|---|---|
| | Potency | Potency |
| ISVD ID | (M) | (M) |
| A0294037F03 | 2.69E−08 | 3.37E−08 |
| A029404472 | 7.45E−09 | 8.41E−09 |
| A029404473 | 3.43E−09 | 3.76E−09 |
| A029405164 | nd | 1.92E−09 |
| A029405175 | 5.97E−09 | 1.04E−08 |

nd: not determined

### 5.20 Example 11: Sequence Optimization of ISVD A0294041A11

### 5.20.1 Sequence Optimization

ISVD A0294041A11 was further sequence optimized to improve chemical stability, to reduce binding by pre-existing antibodies to the ISVD and to make the ISVD more human like. The optimized sequences are depicted in Tables A-3.

### 5.20.2 Off-Rate Analysis of the Sequence Optimized ISVDs

The sequence optimized ISVDs were subjected to off-rate analysis towards human and cyno PD-1 by Surface Plasmon Resonance (SPR) on a ProteOn XPR36 (Bio-Rad Laboratories), SPR-32 Bruker and an Cytiva Biacore 8K+ instrument. The experiment was performed at 25° C. or 37° C. Thereto, recombinant hPD1::Halotag-histag and cPD-1::Halotag-histag (in house produced) were immobilized on GLC sensorchip (Bio-Rad Laboratories), or a High-Capacity Amine chip (Bruker) or on a CM5 chip (GE Healthcare Life Sciences) via amine coupling, using EDC and NHS chemistry (running buffer used: HBS-EP+, pH7.4). Periplasmic extracts were diluted 1:10 in running buffer (HBS-EP+, pH7.4) and they were injected over the sensor surfaces (association: 120 s, dissociation: 900 s, 45 μL/min). Between sample injections, the surfaces were regenerated with 100 mM HCL (Merck Millipore). Several buffer blanks were injected for double referencing. Data was double referenced by subtracting a reference ligand lane and a blank buffer injection. Processed sensorgrams were analysed based on the 1:1 interaction model (Langmuir binding model) using ProteOn Manager 3.1.0 software (Bio-Rad Laboratories) or using SPR-32 Analyzer 3.2 analysis software or using the Biacore Insight Evaluation software.

The off-rate values for the sequence optimized variants are depicted in Tables B-21B and B-22.

TABLE B-21B
Off-rate values for the sequence optimized variants
of ISVD A0294041A11 measured at 25° C.

| | cPD-1 | hPD-1 |
|---|---|---|
| | koff | koff |
| ISVD ID | (1/s) | (1/s) |
| A0294041A11 | 5.30E−04 | 4.10E−04 |
| A029402118 | 1.30E−02 | 2.40E−02 |
| A029402119 | 1.40E−02 | 2.10E−02 |
| A029402120 | 1.50E−02 | 2.30E−02 |
| A029402121 | 1.30E−02 | 2.00E−02 |
| A029402122 | 1.50E−02 | 2.40E−02 |
| A029402125 | 5.00E−01 | 5.00E−01 |
| A029402127 | 1.10E−02 | 2.30E−02 |
| A029402129 | 2.30E−02 | 5.00E−01 |
| A029402130 | 1.30E−02 | 2.00E−02 |
| A029402139 | 1.40E−02 | 2.60E−02 |
| A029402143 | 5.00E−01 | 5.00E−01 |
| A029402144 | 5.00E−01 | 5.00E−01 |
| A029402148 | 8.00E−02 | 2.50E−01 |
| A029402153 | 2.70E−03 | 3.20E−03 |
| A029402154 | 3.10E−03 | 3.60E−03 |
| A029402157 | 2.50E−03 | 3.40E−03 |
| A029402158 | 4.10E−03 | 5.60E−03 |
| A029402159 | 2.20E−03 | 3.00E−03 |
| A029402160 | 6.70E−03 | 9.70E−03 |
| A029402161 | 2.50E−03 | 2.90E−03 |
| A029402162 | 2.30E−03 | 2.70E−03 |
| A029402167 | 2.30E−03 | 2.90E−03 |
| A029402170 | 6.60E−03 | 9.10E−03 |
| A029402177 | 7.90E−03 | 4.20E−03 |
| A029402178 | 2.10E−03 | 4.30E−03 |
| A029402181 | 1.90E−02 | 3.00E−02 |
| A029402186 | 4.00E−03 | 5.20E−03 |
| A029402189 | 6.00E−03 | 1.20E−02 |
| A029402190 | 4.40E−03 | 5.90E−03 |
| A029402191 | 3.40E−03 | 4.50E−03 |
| A029402193 | 3.20E−03 | 3.90E−03 |
| A029402197 | 6.30E−03 | 5.40E−03 |
| A029402198 | 3.30E−03 | 3.90E−03 |
| A029402199 | 2.30E−03 | 2.90E−03 |
| A029402200 | 7.30E−03 | 6.60E−03 |
| A029402202 | 1.00E−02 | 2.50E−02 |
| A029402204 | 3.90E−03 | 5.80E−03 |
| A029402206 | 5.40E−03 | 1.20E−02 |
| A029402207 | 3.90E−03 | 5.30E−03 |
| A029402209 | 6.40E−03 | 9.90E−03 |
| A029402210 | 2.00E−02 | 9.70E−02 |
| A029402213 | 9.70E−03 | 2.40E−01 |
| A029402225 | 2.60E−03 | 3.70E−03 |
| A029402230 | 1.90E−03 | 2.00E−03 |
| A029402231 | 5.00E−01 | 1.30E−03 |
| A029402232 | 2.40E−02 | 2.20E−02 |
| A029402233 | 5.00E−01 | 1.90E−03 |
| A029402235 | 5.00E−01 | 5.00E−01 |
| A029402236 | 1.10E−03 | 2.50E−03 |
| A029402238 | 5.00E−01 | 5.00E−01 |
| A029402244 | 1.10E−02 | 5.00E−01 |
| A029402248 | 5.00E−01 | 5.00E−01 |
| A029402249 | 4.00E−03 | 3.30E−03 |
| A029402252 | 2.20E−03 | 2.50E−03 |
| A029402256 | 5.00E−01 | 5.00E−01 |
| A029402259 | 5.00E−01 | 5.00E−01 |
| A029402265 | 2.20E−03 | 3.00E−03 |
| A029402270 | 1.20E−03 | 8.30E−04 |
| A029402271 | 1.20E−03 | 7.40E−04 |
| A029402273 | 5.00E−01 | 5.00E−01 |
| A029402274 | 7.40E−04 | 5.00E−01 |
| A029402275 | nd | 1.90E−03 |
| A029402276 | nd | 1.50E−03 |
| A029402280 | nd | 7.10E−04 |
| A029402281 | 1.80E−03 | 1.40E−03 |
| A029402283 | 5.00E−01 | 5.00E−01 |
| A029402291 | 7.60E−04 | 7.80E−04 |
| A029402292 | 5.70E−04 | nd |
| A029402304 | 1.30E−04 | 1.40E−04 |
| A029402307 | 5.00E−01 | 3.40E−03 |
| A029402308 | 5.00E−01 | 5.00E−01 |
| A029402309 | 2.90E−02 | 6.00E−03 |
| A029402311 | 5.00E−01 | 1.20E−01 |
| A029402312 | 5.80E−03 | 5.40E−03 |
| A029402313 | 5.00E−01 | 2.90E−01 |
| A029402315 | 1.60E−02 | 5.00E−01 |
| A029402316 | 5.40E−03 | 5.00E−01 |
| A029402319 | 5.00E−01 | 5.00E−01 |
| A029402324 | 5.00E−01 | 7.70E−03 |
| A029402327 | 5.00E−01 | 5.00E−01 |
| A029402332 | 2.80E−02 | 5.00E−01 |
| A029402334 | 2.40E−03 | 6.60E−04 |
| A029402344 | 5.00E−01 | 5.00E−01 |
| A029402346 | 5.00E−01 | 1.70E−03 |
| A029402347 | 3.00E−03 | 8.00E−03 |
| A029402349 | 5.00E−01 | 3.10E−03 |
| A029402350 | 3.20E−02 | 5.00E−01 |
| A029402351 | 1.00E−03 | 1.30E−03 |
| A029402352 | 1.10E−03 | 1.50E−03 |
| A029402354 | 1.10E−03 | 1.90E−03 |
| A029402362 | 1.90E−03 | 1.50E−03 |
| A029402363 | 1.00E−03 | 1.20E−03 |
| A029402365 | 6.60E−02 | 5.00E−01 |
| A029402366 | 9.40E−02 | 5.30E−03 |
| A029402374 | 2.60E−03 | 3.50E−03 |
| A029402377 | 1.80E−03 | nd |
| A029402383 | 1.20E−02 | 7.20E−03 |
| A029402386 | 3.60E−03 | nd |
| A029402390 | 1.00E−02 | 4.50E−03 |
| A029402393 | nd | 1.50E−03 |
| A029402394 | 4.60E−02 | 1.30E−03 |
| A029402395 | 1.80E−01 | 1.20E−03 |
| A029402399 | 2.80E−03 | 1.10E−03 |
| A029402420 | 2.30E−03 | 2.30E−03 |
| A029402421 | 2.00E−03 | 2.10E−03 |
| A029402424 | 4.80E−03 | 4.20E−03 |
| A029402427 | 2.50E−03 | 2.90E−03 |
| A029402428 | nd | 1.70E−03 |
| A029402429 | 6.00E−03 | 2.70E−03 |
| A029402430 | 2.90E−03 | 3.40E−03 |
| A029402432 | 2.10E−02 | 2.70E−03 |
| A029402434 | 2.70E−03 | 1.50E−03 |
| A029402436 | 3.30E−02 | 5.00E−01 |
| A029402437 | 4.30E−03 | nd |
| A029402447 | 3.70E−03 | 5.30E−03 |
| A029402448 | 1.40E−03 | 2.10E−03 |
| A029402449 | 2.80E−03 | 3.50E−03 |
| A029402456 | 1.60E−03 | 1.20E−03 |
| A029402457 | 1.80E−03 | 1.30E−03 |
| A029402458 | 3.00E−03 | 2.00E−02 |
| A029402462 | 1.90E−03 | 2.30E−03 |
| A029402463 | 2.20E−03 | 5.10E−02 |
| A029402464 | 3.90E−02 | 3.90E−03 |
| A029402465 | 2.20E−03 | 5.00E−01 |
| A029402467 | 2.10E−03 | 7.80E−03 |
| A029402470 | 3.00E−03 | 2.00E−03 |
| A029402471 | 1.10E−01 | 5.00E−01 |
| A029402472 | 1.30E−02 | 3.00E−02 |
| A029402474 | 2.20E−03 | 1.50E−03 |
| A029402477 | 8.60E−03 | 1.40E−03 |
| A029402486 | 6.10E−02 | 5.00E−01 |
| A029402487 | 5.00E−01 | 5.00E−01 |
| A029402491 | 3.70E−03 | 6.40E−03 |
| A029402505 | 8.50E−04 | 1.50E−03 |
| A029402514 | 7.70E−04 | 1.00E−03 |
| A029402528 | 5.60E−04 | 7.50E−04 |
| A029402530 | 2.80E−03 | 4.00E−03 |
| A029402531 | 2.10E−03 | 5.00E−01 |
| A029402532 | 1.30E−03 | 2.00E−03 |
| A029402533 | 2.00E−03 | 3.40E−03 |
| A029402534 | 1.60E−03 | 5.00E−01 |
| A029402535 | 5.00E−01 | 5.00E−01 |
| A029402537 | 1.50E−03 | 2.60E−03 |
| A029402538 | 1.10E−03 | 1.90E−03 |
| A029402540 | 1.20E−03 | 2.00E−03 |
| A029402542 | 2.00E−03 | 5.00E−01 |
| A029402546 | 1.30E−03 | 2.20E−03 |
| A029402548 | 1.20E−03 | 2.00E−03 |
| A029402550 | 2.10E−03 | 3.70E−03 |
| A029402552 | 2.30E−03 | 3.90E−03 |
| A029402553 | 1.00E−02 | 6.40E−02 |
| A029402570 | 6.70E−04 | 5.00E−01 |
| A029402571 | 1.70E−03 | 3.10E−03 |
| A029402572 | 3.80E−03 | 7.30E−03 |
| A029402573 | 1.30E−03 | 2.60E−03 |
| A029402574 | 1.40E−03 | 2.80E−03 |
| A029402575 | 1.00E−03 | 1.80E−03 |
| A029402576 | 2.00E−03 | 3.50E−03 |
| A029402577 | 1.90E−03 | 3.00E−03 |
| A029402579 | 3.20E−03 | 6.20E−03 |
| A029402581 | 2.00E−03 | 3.80E−03 |
| A029402582 | 3.00E−03 | 5.10E−03 |
| A029402589 | 3.70E−03 | 6.90E−03 |
| A029402593 | 4.00E−03 | 5.00E−01 |
| A029402594 | 2.80E−03 | 5.00E−03 |
| A029402600 | 2.70E−03 | 4.80E−03 |
| A029402603 | 1.10E−03 | 2.30E−03 |
| A029402608 | 1.10E−03 | 1.90E−03 |
| A029402612 | 1.30E−03 | 1.90E−03 |
| A029402613 | 2.00E−03 | 3.30E−03 |
| A029402614 | 3.30E−03 | 6.10E−03 |
| A029402615 | 1.30E−03 | 2.00E−03 |
| A029402616 | 1.00E−03 | 2.00E−03 |
| A029402617 | 1.30E−03 | 2.00E−03 |
| A029402619 | 1.20E−03 | 1.90E−03 |
| A029402622 | 1.40E−03 | 2.50E−03 |
| A029402623 | 1.90E−03 | 3.20E−03 |
| A029402624 | 1.20E−03 | 1.80E−03 |
| A029402625 | 1.20E−03 | 2.10E−03 |
| A029402631 | 1.50E−03 | 2.60E−03 |
| A029402635 | 1.10E−03 | 1.80E−03 |
| A029402643 | 2.10E−03 | 3.70E−03 |
| A029402652 | 5.30E−03 | 1.20E−02 |
| A029402653 | 1.20E−03 | 2.40E−03 |
| A029402655 | 1.20E−03 | 2.10E−03 |
| A029402656 | 6.70E−03 | 1.20E−02 |
| A029402659 | 3.00E−02 | 5.00E−01 |
| A029402660 | 1.10E−02 | 3.10E−01 |
| A029402661 | 2.20E−03 | 4.10E−03 |
| A029402664 | 2.70E−03 | 5.00E−03 |
| A029402666 | 1.70E−03 | 3.80E−03 |
| A029402667 | 1.90E−03 | 4.60E−03 |
| A029402670 | 4.30E−03 | 8.30E−03 |
| A029402672 | 4.80E−03 | 1.50E−02 |
| A029402674 | 4.40E−03 | 1.10E−02 |
| A029402678 | 1.40E−03 | 3.40E−03 |
| A029402686 | 3.70E−03 | 7.50E−03 |
| A029402689 | 1.60E−03 | 4.00E−03 |
| A029402690 | 1.90E−03 | 5.70E−03 |
| A029402692 | 2.50E−03 | 1.20E−02 |
| A029402693 | 1.40E−03 | 3.80E−03 |
| A029402694 | 6.70E−03 | 3.90E−03 |
| A029402695 | 2.50E−03 | 5.00E−03 |
| A029402696 | 2.30E−03 | 4.20E−03 |
| A029402698 | 3.00E−03 | 8.70E−03 |
| A029402699 | 2.90E−03 | 5.80E−03 |
| A029402700 | 4.00E−03 | 8.60E−03 |
| A029402704 | 2.60E−03 | 7.20E−03 |
| A029402706 | 6.30E−03 | 3.30E−02 |
| A029402709 | 3.10E−03 | 7.80E−03 |
| A029402712 | 3.90E−03 | 1.20E−02 |
| A029402715 | 3.70E−03 | 1.00E−02 |
| A029402725 | 1.10E−03 | 2.60E−03 |
| A029402726 | 1.10E−03 | 2.20E−03 |
| A029402727 | 1.10E−03 | 2.60E−03 |
| A029402728 | 1.30E−03 | 2.90E−03 |
| A029402729 | 1.80E−03 | 3.10E−03 |
| A029402731 | 1.10E−03 | 1.80E−03 |
| A029402734 | 5.00E−01 | 5.00E−01 |
| A029402735 | 1.60E−03 | 3.20E−03 |
| A029402736 | 1.80E−03 | 3.50E−03 |
| A029402737 | 1.10E−03 | 2.60E−03 |
| A029402738 | 1.10E−03 | 2.20E−03 |
| A029402749 | 1.60E−03 | 3.40E−03 |
| A029402752 | 1.80E−03 | 3.40E−03 |
| A029402753 | 1.60E−03 | 3.40E−03 |
| A029402758 | 1.30E−03 | 2.20E−03 |
| A029402764 | 1.50E−03 | 3.20E−03 |
| A029402765 | 1.90E−03 | 4.90E−03 |
| A029402766 | 1.70E−03 | 4.40E−03 |
| A029402767 | 1.90E−03 | 2.90E−01 |
| A029402768 | 1.80E−03 | 8.30E−03 |
| A029402769 | 1.20E−03 | 1.80E−03 |
| A029402773 | 2.50E−03 | 3.30E−03 |
| A029402774 | 3.00E−03 | 5.20E−03 |
| A029402775 | 2.80E−03 | 4.40E−03 |
| A029402779 | 2.10E−03 | 5.40E−03 |
| A029402782 | 2.90E−03 | 5.50E−03 |
| A029402786 | 2.10E−03 | 3.50E−03 |
| A029402789 | 2.00E−03 | 3.40E−03 |
| A029402797 | 2.00E−03 | 3.50E−03 |
| A029402798 | 1.60E−03 | 2.90E−03 |
| A029402802 | 1.30E−03 | 5.80E−03 |
| A029402803 | 1.20E−03 | 2.50E−03 |
| A029402804 | 1.20E−03 | 4.10E−03 |
| A029402805 | 1.30E−03 | 6.70E−03 |
| A029402806 | 1.20E−03 | 1.50E−03 |
| A029402808 | 1.80E−03 | 2.80E−03 |
| A029402810 | 1.80E−03 | 3.10E−03 |
| A029402811 | 2.40E−03 | 4.20E−03 |
| A029402812 | 5.00E−01 | 5.00E−01 |
| A029402813 | 2.30E−03 | 4.90E−03 |
| A029402815 | 1.30E−03 | 1.60E−03 |
| A029402816 | 2.90E−03 | 2.70E−03 |
| A029402817 | 1.30E−03 | 4.30E−03 |
| A029402818 | 1.30E−03 | 2.00E−03 |
| A029402824 | 2.30E−03 | 4.30E−03 |
| A029402827 | 2.60E−03 | 4.40E−03 |
| A029402829 | 1.40E−03 | 1.80E−03 |
| A029402831 | 2.90E−03 | 1.00E−02 |
| A029402840 | 1.40E−03 | 1.50E−02 |
| A029402841 | 1.10E−03 | 1.30E−02 |
| A029402842 | 1.40E−03 | 1.70E−02 |
| A029402843 | 1.20E−03 | 1.10E−01 |
| A029402844 | 1.50E−03 | 1.50E−03 |
| A029402846 | 1.80E−03 | 3.70E−03 |
| A029402847 | 1.60E−03 | 2.90E−03 |
| A029402848 | 1.90E−03 | 3.40E−03 |
| A029402850 | 2.40E−03 | 4.60E−03 |
| A029402853 | 1.40E−03 | 9.10E−03 |
| A029402856 | 1.10E−03 | 3.00E−01 |
| A029402857 | 1.30E−03 | 6.30E−03 |
| A029402861 | 4.90E−03 | 1.50E−02 |
| A029402864 | 5.50E−03 | 2.20E−02 |
| A029402868 | 2.70E−03 | 8.50E−03 |
| A029402876 | 1.80E−03 | 3.40E−03 |
| A029402877 | 1.00E−03 | 1.60E−03 |
| A029402878 | 9.90E−04 | 1.70E−03 |
| A029402879 | 9.50E−04 | 1.50E−03 |
| A029402880 | 9.60E−04 | 1.70E−03 |
| A029402882 | 9.70E−04 | 1.80E−03 |
| A029402883 | 9.20E−04 | 1.60E−03 |
| A029402884 | 9.80E−04 | 1.70E−03 |
| A029402885 | 1.00E−03 | 1.90E−03 |
| A029402886 | 9.80E−04 | 1.80E−03 |
| A029402888 | 9.90E−04 | 2.00E−03 |
| A029402889 | 9.80E−04 | 1.70E−03 |
| A029402890 | 9.70E−04 | 1.60E−03 |
| A029402892 | 9.50E−04 | 1.60E−03 |
| A029402894 | 9.60E−04 | 1.70E−03 |
| A029402895 | 1.20E−03 | 2.10E−03 |
| A029402900 | 1.20E−03 | 2.10E−03 |
| A029402902 | 1.10E−03 | 2.00E−03 |
| A029402904 | 1.00E−03 | 1.90E−03 |
| A029402905 | 9.90E−04 | 1.90E−03 |
| A029402906 | 1.00E−03 | 1.90E−03 |
| A029402907 | 9.90E−04 | 1.90E−03 |
| A029402908 | 1.00E−03 | 2.00E−03 |
| A029402909 | 1.00E−03 | 2.00E−03 |
| A029402912 | 1.00E−03 | 2.00E−03 |
| A029402920 | 1.00E−03 | 2.00E−03 |
| A029402921 | 1.10E−03 | 2.20E−03 |
| A029402926 | 1.00E−03 | 2.20E−03 |
| A029402928 | 1.00E−03 | 2.30E−03 |
| A029402933 | 1.10E−03 | 1.80E−03 |
| A029402934 | 9.70E−04 | 1.40E−03 |
| A029402935 | 9.40E−04 | 1.30E−03 |
| A029402936 | 1.10E−03 | 1.90E−03 |
| A029402937 | 9.40E−04 | 1.50E−03 |
| A029402938 | 1.10E−03 | 1.70E−03 |
| A029402939 | 1.10E−03 | 1.70E−03 |
| A029402941 | 1.10E−03 | 1.80E−03 |
| A029402943 | 9.00E−04 | 1.30E−03 |
| A029402946 | 9.20E−04 | 1.70E−03 |
| A029402949 | 1.00E−03 | 1.80E−03 |
| A029402954 | 8.80E−04 | 1.20E−03 |
| A029402955 | 9.30E−04 | 1.70E−03 |
| A029402962 | 9.40E−04 | 1.40E−03 |
| A029402965 | 1.10E−03 | 1.80E−03 |
| A029402970 | 1.10E−03 | 2.00E−03 |
| A029402971 | 1.20E−03 | 2.20E−03 |
| A029402972 | 9.90E−04 | 2.00E−03 |
| A029402975 | 1.10E−03 | 1.80E−03 |
| A029402977 | 1.30E−03 | 2.20E−03 |
| A029402979 | 1.80E−03 | 3.20E−03 |
| A029402983 | 1.10E−03 | 1.60E−03 |
| A029402985 | 1.30E−03 | 2.30E−03 |
| A029402986 | 1.60E−03 | 1.60E−03 |
| A029402988 | 1.30E−03 | 1.80E−03 |
| A029402991 | 1.30E−03 | 2.30E−03 |
| A029402996 | 1.10E−03 | 2.20E−03 |
| A029403009 | 5.00E−01 | 5.00E−01 |
| A029403010 | 4.50E−01 | 5.00E−01 |
| A029403011 | 3.20E−03 | 2.70E−03 |
| A029403012 | 3.80E−03 | 2.00E−03 |
| A029403013 | 1.80E−03 | 1.70E−03 |
| A029403014 | 1.30E−03 | 2.30E−03 |
| A029403015 | 5.00E−01 | 5.00E−01 |
| A029403016 | 5.00E−01 | 5.00E−01 |
| A029403020 | 5.00E−01 | 5.00E−01 |
| A029403023 | 2.10E−02 | 5.00E−01 |
| A029403025 | 2.10E−03 | 1.70E−03 |
| A029403026 | 5.00E−01 | 5.00E−01 |
| A029403028 | 3.70E−03 | 6.80E−03 |
| A029403033 | 5.00E−01 | 5.00E−01 |
| A029403041 | 5.00E−01 | 5.00E−01 |
| A029403046 | 5.00E−01 | 5.00E−01 |
| A029403048 | 8.80E−03 | 2.10E−02 |
| A029403050 | 5.00E−01 | 5.00E−01 |
| A029403051 | 5.00E−01 | 5.00E−01 |
| A029403052 | 5.00E−01 | 5.00E−01 |
| A029403054 | 5.00E−01 | 5.00E−01 |
| A029403057 | 8.70E−03 | 2.10E−02 |
| A029403060 | 2.00E−03 | 1.50E−03 |
| A029403062 | 2.00E−03 | 1.40E−03 |
| A029403064 | 5.00E−01 | 5.00E−01 |
| A029403070 | 3.20E−03 | 2.00E−03 |
| A029403072 | 8.40E−03 | 5.50E−03 |
| A029403073 | 5.00E−01 | 5.00E−01 |
| A029403078 | 1.20E−02 | 3.40E−02 |
| A029403080 | 7.60E−03 | 1.40E−02 |
| A029403081 | 5.00E−01 | 5.00E−01 |
| A029403082 | 5.00E−01 | 5.00E−01 |
| A029403083 | 3.20E−03 | 4.90E−03 |
| A029403084 | 6.60E−03 | 1.30E−02 |
| A029403086 | 5.00E−01 | 5.00E−01 |
| A029403087 | 5.00E−01 | 5.00E−01 |
| A029403088 | 5.00E−01 | 5.00E−01 |
| A029403093 | 5.00E−01 | 5.00E−01 |
| A029403096 | 5.00E−01 | 5.00E−01 |
| A029403099 | 4.30E−03 | 7.20E−03 |
| A029403104 | 5.00E−01 | 5.00E−01 |
| A029403106 | 5.00E−01 | 5.00E−01 |
| A029403107 | 1.60E−02 | 1.30E−01 |
| A029403119 | 2.00E−03 | 1.00E−02 |
| A029403120 | nd | 1.70E−03 |
| A029403121 | 2.00E−03 | 4.10E−03 |
| A029403123 | 5.00E−01 | 5.00E−01 |
| A029403126 | 5.00E−01 | 2.70E−03 |
| A029403127 | 8.60E−03 | 1.90E−02 |
| A029403128 | 5.00E−01 | 5.00E−01 |
| A029403129 | 3.30E−03 | 5.00E−01 |
| A029403130 | 2.40E−03 | 1.60E−03 |
| A029403131 | 2.40E−03 | 1.70E−03 |
| A029403132 | 4.50E−03 | 6.50E−03 |
| A029403133 | 5.00E−01 | 5.00E−01 |
| A029403134 | 2.40E−03 | 1.80E−03 |
| A029403139 | 5.00E−01 | 5.00E−01 |
| A029403141 | 5.00E−01 | 5.00E−01 |
| A029403148 | 2.60E−03 | 2.00E−03 |
| A029403150 | 5.00E−01 | 5.00E−01 |
| A029403158 | 5.00E−01 | 5.00E−01 |
| A029403159 | 7.40E−03 | 5.00E−01 |
| A029403162 | 5.00E−01 | 5.00E−01 |
| A029403165 | 5.00E−01 | 5.00E−01 |
| A029403166 | 1.30E−01 | 5.00E−01 |
| A029403167 | 4.30E−02 | 5.00E−01 |
| A029403168 | 4.40E−02 | 5.00E−01 |
| A029403169 | 5.00E−01 | 5.00E−01 |
| A029403170 | 4.70E−03 | 1.70E−03 |
| A029403171 | 1.70E−03 | 1.20E−03 |
| A029403172 | 5.00E−01 | 5.00E−01 |
| A029403176 | 8.80E−03 | 5.00E−01 |
| A029403177 | 5.00E−01 | 5.00E−01 |
| A029403183 | 5.00E−01 | 5.00E−01 |
| A029403188 | nd | 1.40E−03 |
| A029403198 | 3.40E−03 | 6.60E−03 |
| A029403199 | 2.30E−03 | 4.30E−03 |
| A029403201 | 3.00E−03 | 4.90E−03 |
| A029403203 | 1.20E−03 | 2.90E−03 |
| A029403206 | 2.40E−03 | 4.30E−03 |
| A029403208 | 3.70E−03 | 6.40E−03 |
| A029403210 | 5.60E−03 | 5.00E−01 |
| A029403211 | 6.50E−03 | 9.90E−03 |
| A029403213 | 4.00E−03 | 1.80E−03 |
| A029403226 | 1.80E−03 | 2.90E−03 |
| A029403229 | 3.80E−03 | 5.80E−03 |
| A029403232 | 1.70E−03 | 2.50E−03 |
| A029403234 | 6.70E−03 | 1.30E−02 |
| A029403235 | 1.30E−03 | 1.30E−03 |
| A029403236 | 7.00E−04 | 6.20E−04 |
| A029403237 | 1.30E−03 | 1.20E−03 |
| A029403239 | 2.60E−03 | 2.70E−03 |
| A029403240 | 1.50E−03 | 1.80E−03 |
| A029403243 | 5.70E−03 | 6.40E−03 |
| A029403245 | 1.90E−03 | 1.60E−03 |
| A029403246 | 4.40E−04 | 4.70E−04 |
| A029403247 | 5.00E−01 | 5.00E−01 |
| A029403248 | 1.00E−02 | 2.30E−01 |
| A029403249 | 5.00E−01 | nd |
| A029403252 | 1.20E−03 | 1.30E−03 |
| A029403255 | 2.30E−03 | 2.10E−03 |
| A029403258 | 6.90E−03 | 1.20E−02 |
| A029403263 | 1.30E−03 | 1.20E−03 |
| A029403264 | 5.70E−03 | 1.60E−02 |
| A029403265 | 5.00E−01 | 5.00E−01 |
| A029403267 | 1.30E−02 | 5.00E−01 |
| A029403268 | 1.30E−02 | 5.00E−01 |
| A029403270 | 5.60E−03 | 1.10E−02 |
| A029403271 | 4.60E−03 | 7.60E−03 |
| A029403272 | 6.40E−03 | 1.10E−02 |
| A029403273 | 7.10E−03 | 1.10E−02 |
| A029403274 | 1.50E−03 | 2.30E−03 |
| A029403276 | 6.60E−03 | 1.20E−02 |
| A029403278 | 2.60E−03 | 4.10E−03 |
| A029403283 | 3.30E−03 | 5.70E−03 |
| A029403294 | 3.60E−03 | 5.50E−03 |
| A029403297 | 1.20E−03 | 1.60E−03 |
| A029403298 | 1.10E−03 | 1.90E−03 |
| A029403299 | 1.30E−03 | 2.30E−03 |
| A029403300 | 1.20E−03 | 1.60E−03 |
| A029403303 | 1.20E−03 | 1.60E−03 |
| A029403304 | 1.20E−03 | 1.70E−03 |
| A029403305 | 1.40E−03 | 2.30E−03 |
| A029403306 | 2.20E−03 | 3.40E−03 |
| A029403308 | 1.30E−03 | 1.90E−03 |
| A029403309 | 1.20E−03 | 1.90E−03 |
| A029403310 | 3.30E−03 | 5.80E−03 |
| A029403314 | 2.80E−03 | 4.50E−03 |
| A029403316 | 1.50E−03 | 2.50E−03 |
| A029403323 | 1.40E−03 | 2.20E−03 |
| A029403328 | 1.60E−03 | 2.70E−03 |
| A029403335 | 1.20E−03 | 2.20E−03 |
| A029403336 | 1.10E−03 | 1.60E−03 |
| A029403338 | 1.30E−03 | 1.70E−03 |
| A029403339 | 1.50E−03 | 3.10E−03 |
| A029403342 | 1.20E−03 | 1.70E−03 |
| A029403344 | 1.30E−03 | 2.20E−03 |
| A029403345 | 1.40E−03 | 2.00E−03 |
| A029403347 | 1.40E−03 | 2.00E−03 |
| A029403349 | 1.20E−03 | 1.90E−03 |
| A029403351 | 1.50E−03 | 2.80E−03 |
| A029403352 | 1.30E−03 | 2.30E−03 |
| A029403353 | 1.40E−03 | 2.40E−03 |
| A029403354 | 1.20E−03 | 1.90E−03 |
| A029403360 | 1.30E−03 | 2.10E−03 |
| A029403364 | 1.50E−02 | 5.00E−01 |
| A029403374 | 2.10E−03 | 3.00E−03 |
| A029403375 | 2.40E−03 | 5.10E−03 |
| A029403377 | 2.60E−03 | 5.60E−03 |
| A029403378 | 2.40E−03 | 2.40E−02 |
| A029403379 | 3.60E−03 | 3.30E−02 |
| A029403380 | 2.50E−03 | 4.40E−03 |
| A029403381 | 5.60E−03 | 1.10E−02 |
| A029403383 | 5.00E−01 | 5.00E−01 |
| A029403387 | 4.70E−03 | 1.20E−02 |
| A029403388 | 1.90E−03 | 4.00E−03 |
| A029403389 | 4.40E−03 | 1.20E−02 |
| A029403391 | 1.20E−02 | 6.70E−02 |
| A029403394 | 3.00E−02 | 5.00E−01 |
| A029403406 | 3.30E−03 | 4.90E−03 |
| A029403412 | 1.30E−03 | 3.90E−03 |
| A029403413 | 1.40E−03 | 3.40E−03 |
| A029403414 | 2.10E−03 | 5.50E−03 |
| A029403415 | 2.30E−03 | 6.60E−03 |
| A029403416 | 2.20E−03 | 5.10E−03 |
| A029403417 | 2.60E−03 | 9.60E−03 |
| A029403420 | 2.50E−03 | 5.30E−03 |
| A029403421 | 2.40E−03 | 4.20E−03 |
| A029403423 | 3.00E−03 | 5.40E−03 |
| A029403424 | 1.70E−03 | 4.30E−03 |
| A029403426 | 2.10E−03 | 4.80E−03 |
| A029403427 | 2.40E−03 | 6.00E−03 |
| A029403440 | 2.60E−03 | 4.80E−03 |
| A029403444 | 5.00E−01 | 5.00E−01 |
| A029403445 | 5.00E−01 | 5.00E−01 |
| A029403446 | 2.20E−03 | 4.10E−03 |
| A029403451 | 1.40E−02 | 5.00E−01 |
| A029403452 | 6.70E−03 | 5.00E−01 |
| A029403453 | 3.70E−03 | 1.90E−03 |
| A029403454 | 3.20E−03 | 2.80E−03 |
| A029403455 | 2.60E−02 | 2.80E−03 |
| A029403456 | 2.30E−03 | 3.40E−03 |
| A029403457 | 3.70E−03 | 5.00E−03 |
| A029403458 | 5.00E−01 | 5.00E−01 |
| A029403459 | 3.80E−03 | 9.10E−03 |
| A029403460 | 2.70E−03 | 1.00E−02 |
| A029403461 | 5.00E−01 | 5.00E−01 |
| A029403463 | 6.30E−03 | 1.50E−02 |
| A029403467 | 4.10E−03 | 6.30E−03 |
| A029403471 | 3.00E−03 | 4.60E−03 |
| A029403474 | 5.90E−03 | 1.40E−02 |
| A029403475 | 3.70E−03 | 4.40E−03 |
| A029403479 | 5.50E−03 | 4.70E−01 |
| A029403480 | 6.30E−03 | 5.00E−01 |
| A029403481 | 3.00E−03 | 1.50E−03 |
| A029403482 | 6.60E−03 | 5.00E−01 |
| A029403484 | 2.90E−03 | 4.20E−03 |
| A029403485 | 5.00E−03 | 7.70E−03 |
| A029403486 | 2.70E−03 | 3.50E−03 |
| A029403487 | 3.90E−03 | 5.20E−03 |
| A029403489 | 5.00E−01 | 5.00E−01 |
| A029403490 | 4.30E−03 | 2.50E−03 |
| A029403492 | 4.40E−03 | 1.90E−03 |
| A029403493 | 5.40E−03 | 1.70E−02 |
| A029403495 | 5.70E−03 | 2.60E−02 |
| A029403512 | 4.50E−03 | 6.40E−03 |
| A029403514 | 3.60E−03 | 5.30E−03 |
| A029403518 | 6.20E−02 | 4.90E−01 |
| A029403519 | 3.40E−03 | 1.40E−03 |
| A029403520 | 9.90E−03 | 2.50E−03 |
| A029403521 | 2.90E−03 | 1.30E−03 |
| A029403522 | 7.60E−03 | 1.40E−03 |
| A029403523 | 4.40E−03 | 1.40E−03 |
| A029403524 | 7.60E−03 | 1.70E−03 |
| A029403525 | 5.00E−01 | 5.00E−01 |
| A029403529 | 5.00E−01 | 2.40E−03 |
| A029403530 | 3.00E−03 | 1.50E−03 |
| A029403532 | 4.20E−03 | 1.60E−03 |
| A029403533 | 5.00E−01 | 1.50E−03 |
| A029403535 | 3.80E−03 | 1.20E−03 |
| A029403538 | 5.00E−01 | 2.10E−03 |
| A029403547 | 5.00E−01 | 5.00E−01 |
| A029403558 | 3.00E−03 | 1.40E−03 |
| A029403559 | 4.80E−03 | 3.00E−03 |
| A029403561 | 5.00E−01 | 1.90E−03 |
| A029403563 | 1.90E−02 | 2.80E−03 |
| A029403564 | 5.00E−01 | 5.00E−01 |
| A029403565 | 1.10E−01 | 5.00E−01 |
| A029403566 | 5.00E−01 | 5.00E−01 |
| A029403568 | 5.00E−01 | 5.00E−01 |
| A029403570 | 5.00E−01 | 1.60E−03 |
| A029403571 | 7.60E−03 | 2.20E−03 |
| A029403572 | 5.00E−01 | 2.60E−01 |
| A029403576 | 6.30E−03 | 5.00E−01 |
| A029403578 | 1.40E−02 | 4.70E−01 |
| A029403580 | 5.90E−03 | 3.20E−01 |
| A029403590 | 1.90E−01 | 5.00E−01 |
| A029403593 | 5.00E−01 | 5.00E−01 |
| A029403598 | 3.90E−02 | 1.70E−01 |
| A029403599 | 5.00E−01 | 5.00E−01 |
| A029403600 | 2.20E−03 | 2.50E−03 |
| A029403601 | 5.00E−01 | 5.00E−01 |
| A029403602 | 5.00E−01 | 2.50E−03 |
| A029403603 | 5.00E−01 | 2.60E−03 |
| A029403606 | 2.60E−02 | 4.30E−02 |
| A029403609 | 4.50E−03 | 5.20E−03 |
| A029403610 | 2.00E−02 | 7.90E−02 |
| A029403615 | 1.00E−02 | 6.90E−03 |
| A029403619 | 1.70E−01 | 1.20E−03 |
| A029403621 | 5.00E−01 | 1.40E−03 |
| A029403632 | 2.50E−03 | 2.70E−03 |
| A029403633 | 5.00E−01 | 5.00E−01 |
| A029403634 | 5.00E−01 | 5.00E−01 |
| A029403636 | 5.00E−01 | 5.00E−01 |
| A029403637 | 1.40E−02 | 9.90E−02 |
| A029403638 | 5.00E−01 | 5.00E−01 |
| A029403640 | 5.00E−01 | 8.00E−02 |
| A029403641 | 5.00E−01 | 5.00E−01 |
| A029403648 | 5.00E−01 | 5.00E−01 |
| A029403650 | 5.00E−01 | 5.00E−01 |
| A029403651 | 5.00E−01 | 5.00E−01 |
| A029403654 | 5.00E−01 | 5.00E−01 |
| A029403660 | 1.00E−02 | 2.10E−02 |
| A029403662 | 5.00E−01 | 1.50E−03 |
| A029403666 | 5.00E−01 | 1.10E−03 |
| A029403667 | 2.40E−02 | 3.00E−03 |
| A029403668 | 5.00E−01 | 5.00E−01 |
| A029403669 | 5.00E−01 | 1.20E−03 |
| A029403670 | 5.00E−01 | 1.10E−03 |
| A029403671 | 5.00E−01 | 1.80E−03 |
| A029403672 | 5.00E−01 | 7.10E−02 |
| A029403673 | 5.00E−01 | 1.40E−03 |
| A029403674 | 3.90E−03 | 7.00E−04 |
| A029403678 | 5.00E−01 | 5.00E−01 |
| A029403679 | 5.00E−01 | 5.00E−01 |
| A029403686 | 2.60E−03 | 5.00E−01 |
| A029403690 | 1.10E−01 | 5.00E−01 |
| A029403701 | 5.00E−01 | 5.00E−01 |
| A029403715 | 5.00E−01 | 1.20E−03 |
| A029403728 | 3.20E−03 | 1.80E−03 |
| A029403765 | 1.50E−03 | 1.40E−03 |
| A029403766 | 1.50E−03 | 1.70E−03 |
| A029403767 | 1.50E−03 | 2.00E−03 |
| A029403768 | 1.50E−03 | 1.70E−03 |
| A029403769 | 1.50E−03 | 1.50E−03 |
| A029403770 | 1.50E−03 | 1.50E−03 |
| A029403771 | 1.50E−03 | 1.40E−03 |
| A029403772 | 1.50E−03 | 1.30E−03 |
| A029403773 | 1.60E−03 | 1.60E−03 |
| A029403774 | 1.60E−03 | 1.70E−03 |
| A029403775 | 1.60E−03 | 2.00E−03 |
| A029403776 | 1.70E−03 | 1.90E−03 |
| A029403777 | 1.60E−03 | 1.60E−03 |
| A029403778 | 1.60E−03 | 1.70E−03 |
| A029403779 | 1.50E−03 | 1.50E−03 |

nd: not determined

TABLE B-22
Off-rate values for the sequence optimized variants
of A0294041A11 measured at 37° C.

| | cPD-1 | hPD-1 |
|---|---|---|
| | koff | koff |
| ISVD ID | (1/s) | (1/s) |
| A029400075 | 1.78E−04 | 1.82E−04 |
| A029400135 | 1.02E−04 | 9.49E−05 |
| A029400136 | 1.02E−03 | 1.30E−03 |
| A029400137 | 1.10E−03 | 1.35E−03 |
| A029400138 | 2.48E−04 | 2.70E−04 |
| A029400139 | 2.52E−04 | 2.78E−04 |
| A029400140 | 1.88E−04 | 2.34E−04 |
| A029400141 | 1.96E−04 | 2.54E−04 |
| A029400142 | 2.42E−04 | 2.65E−04 |
| A029400143 | 7.51E−04 | 9.66E−04 |
| A029400144 | 1.03E−03 | 1.30E−03 |
| A029400145 | 8.27E−04 | 1.04E−03 |
| A029400146 | 9.07E−04 | 1.12E−03 |
| A029400147 | 2.49E−04 | 3.20E−04 |
| A029400148 | 7.30E−04 | 8.89E−04 |
| A029400149 | 9.53E−04 | 1.16E−03 |
| A029400150 | 7.31E−04 | 8.82E−04 |
| A029400151 | 7.36E−04 | 8.94E−04 |
| A029400152 | 9.23E−04 | 1.13E−03 |
| A029400153 | 2.12E−04 | 2.51E−04 |
| A029400154 | 2.14E−04 | 2.46E−04 |
| A029400155 | 2.81E−04 | 3.04E−04 |
| A029400156 | 1.05E−03 | 1.24E−03 |
| A029400157 | 1.80E−04 | 2.03E−04 |
| A029400158 | 2.11E−04 | 2.21E−04 |
| A029400159 | 7.87E−04 | 9.23E−04 |
| A029400160 | 8.58E−04 | 1.04E−03 |
| A029400191 | 7.16E−04 | 7.45E−04 |
| A029400192 | 2.16E−04 | 2.26E−04 |
| A029400193 | 3.22E−04 | 3.36E−04 |
| A029400194 | 2.24E−04 | 2.25E−04 |
| A029400195 | 1.09E−03 | 1.35E−03 |
| A029400196 | 2.65E−04 | 3.08E−04 |
| A029400197 | 2.34E−04 | 2.52E−04 |
| A029404686 | 5.75E−02 | 7.09E−02 |
| A029404687 | 4.16E−02 | 4.15E−02 |
| A029404688 | 4.97E−02 | 6.03E−02 |
| A029404689 | 4.94E−02 | 6.10E−02 |
| A029404690 | 4.05E−02 | 3.98E−02 |
| A029404691 | 4.19E−02 | 4.55E−02 |
| A029404698 | 5.21E−04 | 7.07E−04 |
| A029404699 | 8.37E−04 | 1.14E−03 |
| A029404700 | 1.02E−03 | 1.38E−03 |
| A029404701 | 5.00E−04 | 6.77E−04 |
| A029404702 | 8.05E−04 | 1.11E−03 |
| A029404703 | 9.86E−04 | 1.35E−03 |
| A029404704 | 2.79E−02 | 3.32E−02 |
| A029404705 | 3.35E−02 | 3.50E−02 |
| A029404706 | 3.70E−02 | 3.35E−02 |
| A029404707 | 2.57E−02 | 2.96E−02 |
| A029404708 | 2.71E−02 | 2.91E−02 |

### 5.20.3 Biophysical Characterization of the Sequence Optimized ISVDs

The biophysicial characteristics of the sequence optimized ISVDs of ISVD A0294041A11 were tested in thermal shift assay (Tm) and a temperature of onset of aggregation assay (Tagg).

The thermal shift assay and Tagg assay were performed as described in Example 6.5.1 and 6.5.4 respectively. The Tm and Tagg values obtained for the sequence optimized variants are shown in Table B-23.

TABLE B-23
Biophysical properties of the sequence optimized
variants of ISVD A0294041A11

| | Tm | Tagg |
|---|---|---|
| ISVD ID | (° C.) | (° C.) |
| A0294041A11 | 65.2 | nd |
| A029400075 | 73 | nd |
| A029400137 | 84 | nd |
| A029400138 | 81 | nd |
| A029400140 | 72.9 | 71.4 |
| A029400141 | 78.5 | 75.3 |
| A029400142 | 67.7 | 67.1 |
| A029400145 | 78 | nd |
| A029400146 | 83 | nd |
| A029400147 | 75.1 | 73.3 |
| A029400148 | 79.2 | 70 |
| A029400150 | 73.3 | 66.9 |
| A029400157 | 74 | nd |
| A029400158 | 80 | nd |
| A029400192 | 78 | nd |
| A029400193 | 84 | nd |
| A029400194 | 73 | 69.9 |
| A029400196 | 80 | nd |
| A029400197 | 65.9 | 70.1 |
| A029404018 | 69.6 | 70.6 |
| A029404019 | 74.0 | 76.5 |
| A029404053 | nd | 71.085 |
| A029404055 | nd | 68.205 |
| A029404069 | nd | 78.5 |
| A029404073 | nd | 69.61 |
| A029404075 | nd | 70.735 |
| A029404079 | 62.2 | 61.7 |
| A029404698 | 57.9 | nd |
| A029404699 | 57.9 | nd |
| A029404700 | 57.9 | nd |
| A029404701 | 56.4 | nd |
| A029404702 | 57.9 | nd |
| A029404703 | 56.9 | nd |
| A029404751 | 60 | nd |
| A029404752 | 66 | nd |
| A029404753 | 60 | nd |
| A029404754 | 65 | nd |
| A029404755 | 58 | nd |
| A029404756 | 65 | nd |
| A029404757 | 59 | nd |
| A029404758 | 60 | nd |
| A029404759 | 58 | nd |
| A029404760 | 66 | nd |
| A029404761 | 65 | nd |
| A029404768 | 55.4 | nd |
| A029404778 | 54.5 | nd |
| A029405082 | 56.9 | nd |
| A029405083 | 57.4 | nd |
| A029405084 | 57.4 | nd |
| A029405085 | 55.9 | nd |
| A029405086 | 55.4 | nd |
| A029405087 | 56.4 | nd |
| A029405088 | 57.9 | nd |
| A029405089 | 57.4 | nd |
| A029405090 | 56.9 | nd |
| A029405237 | 61.3 | nd |
| A029405238 | 62.8 | nd |
| A029405239 | 66.2 | nd |
| A029405240 | 70.1 | nd |
| A029405241 | 66.7 | nd |
| A029405242 | 63.3 | nd |
| A029405243 | 62.3 | nd |
| A029405244 | 63.8 | nd |
| A029405246 | 57 | nd |
| A029405247 | 64.2 | nd |
| A029405248 | 68.6 | nd |
| A029405249 | 65.7 | nd |
| A029405250 | 65.7 | nd |
| A029405252 | 65.7 | nd |
| A029405253 | 57.9 | nd |
| A029405254 | 57.4 | nd |
| A029405255 | 70.6 | nd |
| A029405256 | 54.5 | nd |
| A029405257 | 64.2 | nd |
| A029405258 | 62.8 | nd |
| A029405259 | 69.1 | nd |
| A029405260 | 63.8 | nd |
| A029405261 | 57.4 | nd |
| A029405262 | 63.3 | nd |
| A029405263 | 68.1 | nd |
| A029405264 | 64.7 | nd |
| A029405265 | 69.6 | nd |
| A029405266 | 65.2 | nd |
| A029405267 | 63.3 | nd |
| A029405268 | 63.3 | nd |
| A029405269 | 65.2 | nd |
| A029405270 | 62.3 | nd |
| A029405271 | 63.3 | nd |
| A029405272 | 66.2 | nd |
| A029405273 | 66.7 | nd |
| A029405275 | 63.8 | nd |
| A029405276 | 62.3 | nd |
| A029405277 | 55.5 | nd |
| A029405278 | 62.8 | nd |

nd: not determined

### 5.20.4 Characterization of the Sequence Optimized ISVDs for Binding Cells Expressing PD-1

The sequence optimized variants of ISVD A0294041A11 were tested for FACS binding on CHO cell lines expressing human or cyno PD-1 (in house made). The binding FACS was carried out as described in Example 6.1.3. Results are shown in Tables B-24.

TABLE B-24
FACS binding of the sequence optimized variants of ISVD
A0294041A11 on CHO cells expressing human or cyno PD-1

| | cyno PD-1 | human PD-1 |
|---|---|---|
| ISVD ID | EC50 (M) | EC50 (M) |
| A0294041A11 | 6.43E−09 | 1.23E−08 |
| A029404018 | 5.83E−10 | 6.09E−10 |

### 5.20.5 PD-1/PD-L1 Blocking Assay

The sequence optimized variants of ISVD A0294041A11 were tested on a functional NFAT reporter assay, as described above (see 6.1.3). Results of the PD-1/PD-L1 blocking assay are shown in Table B-25.

TABLE B-25
Results of PD-1/PD-L1 blocking assay with sequence
optimized variants of ISVD

| ISVD ID | IC50 (M) |
|---|---|
| A0294041A11 | 5.82E−09 |
| A029400157 | 2.19E−08 |
| A029402230 | 1.35E−09 |
| A029402280 | 1.80E−08 |
| A029402291 | 6.20E−09 |
| A029402304 | 3.82E−08 |
| A029402351 | 2.00E−09 |
| A029402352 | 4.13E−09 |
| A029402354 | 4.07E−09 |
| A029402362 | 2.49E−09 |
| A029402363 | 8.97E−10 |
| A029402377 | 8.51E−09 |
| A029402421 | 1.68E−09 |
| A029402428 | 4.82E−08 |
| A029402448 | 3.85E−08 |
| A029402457 | 1.26E−10 |
| A029402492 | 7.72E−09 |
| A029402496 | 1.49E−09 |
| A029402503 | 1.26E−08 |
| A029402505 | 1.85E−09 |
| A029402514 | 2.35E−09 |
| A029402528 | 2.53E−09 |
| A029402532 | 1.83E−09 |
| A029402538 | 2.23E−09 |
| A029402540 | 1.71E−09 |
| A029402548 | 2.73E−09 |
| A029402608 | 1.84E−09 |
| A029402612 | 1.07E−09 |
| A029402615 | 2.00E−09 |
| A029402616 | 1.66E−09 |
| A029402617 | 1.60E−09 |
| A029402619 | 7.82E−10 |
| A029402624 | 1.57E−09 |
| A029402625 | 2.60E−09 |
| A029402655 | 1.15E−09 |
| A029402731 | 1.30E−09 |
| A029402769 | 3.71E−10 |
| A029402806 | 2.36E−09 |
| A029402818 | 2.71E−09 |
| A029402829 | 2.31E−09 |
| A029402844 | 1.91E−09 |
| A029402877 | 2.27E−09 |
| A029402878 | 1.61E−09 |
| A029402879 | 1.22E−09 |
| A029402880 | 3.71E−09 |
| A029402882 | 1.22E−09 |
| A029402883 | 2.07E−09 |
| A029402884 | 1.41E−09 |
| A029402885 | 4.61E−10 |
| A029402886 | 2.54E−09 |
| A029402888 | 2.33E−09 |
| A029402889 | 2.04E−09 |
| A029402892 | 1.29E−09 |
| A029402894 | 1.17E−09 |
| A029402895 | 2.26E−09 |
| A029402902 | 1.52E−09 |
| A029402904 | 1.78E−09 |
| A029402905 | 1.67E−09 |
| A029402906 | 2.86E−09 |
| A029402907 | 1.61E−09 |
| A029402908 | 1.26E−09 |
| A029402909 | 2.23E−09 |
| A029402912 | 7.67E−10 |
| A029402920 | 1.82E−09 |
| A029402921 | 1.52E−09 |
| A029402933 | 1.46E−09 |
| A029402934 | 1.98E−09 |
| A029402935 | 1.60E−09 |
| A029402936 | 1.10E−09 |
| A029402937 | 1.31E−09 |
| A029402938 | 1.25E−09 |
| A029402939 | 2.32E−09 |
| A029402941 | 2.09E−09 |
| A029402943 | 7.95E−10 |
| A029402946 | 1.38E−09 |
| A029402949 | 1.86E−09 |
| A029402954 | 1.30E−09 |
| A029402955 | 1.36E−09 |
| A029402962 | 3.35E−10 |
| A029402965 | 1.81E−09 |
| A029402970 | 1.16E−09 |
| A029402972 | 1.04E−09 |
| A029402975 | 1.34E−09 |
| A029402983 | 1.30E−09 |
| A029402986 | 1.69E−09 |
| A029402988 | 2.52E−09 |
| A029403060 | 8.24E−09 |
| A029403062 | 3.95E−10 |
| A029403120 | 1.36E−09 |
| A029403235 | 5.72E−09 |
| A029403236 | 2.44E−09 |
| A029403237 | 1.44E−09 |
| A029403240 | 4.16E−09 |
| A029403245 | 3.52E−09 |
| A029403246 | 5.01E−09 |
| A029403252 | 7.72E−09 |
| A029403297 | 4.43E−09 |
| A029403298 | 1.70E−09 |
| A029403300 | 2.26E−09 |
| A029403303 | 4.15E−09 |
| A029403304 | 4.04E−09 |
| A029403308 | 3.15E−09 |
| A029403309 | 5.00E−09 |
| A029403335 | 2.83E−09 |
| A029403336 | 3.23E−09 |
| A029403338 | 2.78E−09 |
| A029403342 | 2.70E−09 |
| A029403345 | 3.76E−09 |
| A029403347 | 3.74E−09 |
| A029403349 | 2.13E−09 |
| A029403354 | 2.27E−09 |
| A029403360 | 3.24E−09 |
| A029403765 | 1.48E−09 |
| A029403766 | 4.81E−09 |
| A029403767 | 5.95E−09 |
| A029403768 | 4.44E−09 |
| A029403769 | 5.65E−09 |
| A029403770 | 3.94E−09 |
| A029403771 | 3.35E−09 |
| A029403772 | 3.12E−09 |
| A029403773 | 2.75E−09 |
| A029403774 | 5.07E−09 |
| A029403775 | 4.61E−09 |
| A029403776 | 7.13E−09 |
| A029403777 | 6.43E−09 |
| A029403778 | 4.31E−09 |
| A029403779 | 3.67E−09 |
| A029404018 | 8.06E−09 |
| A029404053 | 1.38E−08 |
| A029404054 | 5.16E−07 |
| A029404055 | 2.07E−08 |
| A029404056 | 1.61E−08 |
| A029404057 | 4.25E−09 |
| A029404058 | 3.56E−08 |
| A029404059 | 4.79E−08 |
| A029404060 | 1.14E−08 |
| A029404061 | 8.15E−09 |
| A029404062 | 3.01E−08 |
| A029404063 | 2.58E−08 |
| A029404064 | 1.80E−08 |
| A029404065 | 8.28E−08 |
| A029404066 | 6.56E−06 |
| A029404067 | 7.91E−09 |
| A029404068 | 4.77E−08 |
| A029404069 | 2.43E−08 |
| A029404070 | 3.06E−08 |
| A029404071 | 2.67E−08 |
| A029404072 | 2.74E−08 |
| A029404073 | 1.88E−08 |
| A029404074 | 4.28E−08 |
| A029404075 | 2.60E−08 |
| A029404076 | 2.23E−08 |
| A029404077 | 1.56E−08 |
| A029404078 | 3.26E−08 |
| A029404079 | 2.98E−08 |
| A029404080 | 6.58E−05 |
| A029404081 | 1.32E−08 |
| A029404082 | 1.74E−08 |
| A029404083 | 2.02E−08 |
| A029404085 | 2.23E−08 |
| A029404086 | 1.08E−07 |
| A029404087 | 4.96E−08 |
| A029404088 | 2.18E−08 |
| A029404089 | 1.75E−08 |
| A029404090 | 3.50E−08 |
| A029404091 | 2.33E−08 |
| A029404092 | 2.09E−08 |
| A029404093 | 1.59E−08 |
| A029404094 | 2.23E−08 |
| A029404095 | 4.23E−08 |
| A029404096 | 2.13E−08 |
| A029404097 | 2.75E−08 |
| A029404098 | 6.47E−08 |
| A029404099 | 3.56E−08 |
| A029404100 | 1.63E−08 |
| A029404101 | 2.01E−08 |
| A029404102 | 3.96E−08 |
| A029404103 | 1.50E−08 |
| A029404104 | 3.54E−08 |
| A029404105 | 1.72E−08 |
| A029404106 | 3.14E−08 |
| A029404107 | 4.58E−08 |
| A029404108 | 3.15E−08 |
| A029404109 | 1.77E−08 |
| A029404110 | 2.30E−08 |
| A029404111 | 2.84E−08 |
| A029404112 | 3.06E−09 |
| A029404113 | 1.93E−07 |
| A029404114 | 4.46E−08 |
| A029404115 | 2.23E−08 |
| A029404117 | 7.56E−07 |
| A029404122 | 1.68E−07 |
| A029404134 | 9.97E−08 |
| A029404135 | 8.60E−09 |
| A029404142 | 3.63E−11 |
| A029404143 | 6.51E−08 |
| A029404151 | 2.78E−09 |
| A029404153 | 2.45E−09 |
| A029404159 | 3.84E−11 |
| A029404162 | 1.24E−08 |
| A029404164 | 1.88E−09 |
| A029404169 | 2.53E−08 |
| A029404180 | 4.42E−08 |
| A029404181 | 2.86E−08 |
| A029404182 | 5.63E−08 |
| A029404183 | 1.32E−08 |
| A029404184 | 2.49E−08 |
| A029404185 | 2.03E−08 |
| A029404186 | 2.27E−07 |
| A029404187 | 4.54E−08 |
| A029404188 | 6.62E−09 |
| A029404189 | 1.95E−08 |
| A029404191 | 3.26E−08 |
| A029404192 | 3.11E−12 |
| A029404193 | 5.32E−05 |
| A029404194 | 1.52E−10 |
| A029404195 | 3.01E−10 |
| A029404196 | 7.90E−08 |
| A029404197 | 3.51E−08 |
| A029404198 | 3.42E−08 |
| A029404199 | 4.36E−08 |
| A029404200 | 3.21E−08 |
| A029404201 | 6.34E−08 |
| A029404202 | 3.64E−08 |
| A029404203 | 3.41E−08 |
| A029404204 | 1.21E−08 |
| A029404205 | 2.76E−08 |
| A029404206 | 2.62E−08 |
| A029404207 | 3.16E−08 |
| A029404210 | 8.05E−04 |
| A029404211 | 2.61E−08 |
| A029404212 | 2.94E−08 |
| A029404213 | 1.01E−07 |
| A029404214 | 4.51E−08 |
| A029404215 | 2.67E−08 |
| A029404216 | 1.71E−07 |
| A029404217 | 5.90E−08 |
| A029404218 | 3.66E−08 |
| A029404219 | 3.30E−08 |
| A029404220 | 3.80E−08 |
| A029404221 | 1.04E−07 |
| A029404222 | 3.89E−08 |
| A029404223 | 4.62E−06 |
| A029404224 | 5.00E−08 |
| A029404225 | 3.86E−08 |
| A029404226 | 9.99E−08 |
| A029404227 | 4.54E−07 |
| A029404228 | 5.49E−07 |
| A029404229 | 2.36E−07 |
| A029404230 | 4.64E−08 |
| A029404231 | 3.93E−08 |
| A029404232 | 4.32E−08 |
| A029404233 | 4.15E−08 |
| A029404234 | 3.68E−07 |
| A029404235 | 3.99E−03 |
| A029404236 | 1.41E−07 |
| A029404237 | 1.39E−05 |
| A029404238 | 4.72E−08 |
| A029404239 | 1.34E−07 |
| A029404298 | 2.31E−08 |
| A029404299 | 1.69E−08 |
| A029404300 | 2.44E−08 |
| A029404301 | 2.49E−08 |
| A029404302 | 2.64E−08 |
| A029404303 | 2.31E−08 |
| A029404304 | 3.43E−08 |
| A029404305 | 4.40E−08 |
| A029404306 | 7.58E−09 |
| A029404307 | 2.03E−08 |
| A029404308 | 4.39E−08 |
| A029404309 | 2.88E−08 |
| A029404310 | 1.40E−09 |
| A029404311 | 1.06E−09 |
| A029404312 | 3.89E−09 |
| A029404313 | 9.43E−09 |
| A029404314 | 3.19E−08 |
| A029404315 | 2.05E−08 |
| A029404316 | 3.60E−08 |
| A029404317 | 6.18E−08 |
| A029404318 | 3.51E−08 |
| A029404319 | 5.05E−08 |
| A029404320 | 6.24E−08 |
| A029404321 | 3.13E−08 |
| A029404322 | 2.18E−08 |
| A029404323 | 2.58E−08 |
| A029404324 | 2.60E−08 |
| A029404325 | 3.01E−08 |
| A029404326 | 5.81E−04 |
| A029404327 | 9.03E−09 |
| A029404328 | 2.29E−08 |
| A029404329 | 3.43E−08 |
| A029404330 | 2.19E−08 |
| A029404331 | 4.38E−08 |
| A029404332 | 3.25E−08 |
| A029404333 | 3.44E−08 |
| A029404334 | 2.46E−08 |
| A029404335 | 4.28E−08 |
| A029404336 | 4.12E−08 |
| A029404337 | 1.25E−08 |
| A029404338 | 2.81E−08 |
| A029404339 | 7.22E−08 |
| A029404340 | 2.11E−08 |
| A029404341 | 1.55E−08 |
| A029404342 | 1.26E−08 |
| A029404343 | 2.01E−08 |
| A029404344 | 3.62E−08 |
| A029404345 | 2.43E−08 |
| A029404346 | 4.30E−08 |
| A029404347 | 1.75E−08 |
| A029404348 | 3.97E−08 |
| A029404349 | 5.84E−08 |
| A029404350 | 3.06E−08 |
| A029404351 | 1.64E−08 |
| A029404352 | 2.12E−08 |
| A029404353 | 4.01E−08 |
| A029404354 | 1.05E−07 |
| A029404355 | 8.44E−09 |
| A029404356 | 2.58E−08 |
| A029404357 | 3.61E−08 |
| A029404608 | 2.51E−08 |
| A029404609 | 1.99E−07 |
| A029404610 | 1.30E−14 |
| A029404617 | 1.14E−09 |
| A029404618 | 3.10E−08 |
| A029404619 | 3.77E−08 |
| A029404620 | 2.87E−08 |
| A029404751 | 5.11E−08 |
| A029404752 | 3.07E−08 |
| A029404753 | 3.90E−08 |
| A029404754 | 1.61E−08 |
| A029404755 | 6.88E−08 |
| A029404756 | 1.30E−08 |
| A029404757 | 3.71E−08 |
| A029404758 | 4.67E−08 |
| A029404759 | 3.51E−08 |
| A029404760 | 1.16E−08 |
| A029404761 | 2.41E−08 |
| A029404762 | 9.20E−08 |
| A029404768 | 6.18E−08 |
| A029404773 | 2.22E−06 |
| A029404778 | 2.06E−07 |
| A029404784 | 3.33E−05 |
| A029405239 | 1.10E−07 |
| A029405240 | 4.10E−08 |
| A029405241 | 4.41E−07 |
| A029405244 | 4.92E−08 |
| A029405247 | 5.18E−08 |
| A029405248 | 5.22E−08 |
| A029405249 | 9.64E−08 |
| A029405250 | 1.40E−07 |
| A029405252 | 5.74E−08 |
| A029405255 | 4.35E−08 |
| A029405257 | 7.25E−08 |
| A029405259 | 1.20E−08 |
| A029405260 | 3.56E−07 |
| A029405263 | 4.31E−08 |
| A029405264 | 7.44E−08 |
| A029405265 | 4.54E−08 |
| A029405266 | 1.36E−07 |
| A029405269 | 1.38E−07 |
| A029405272 | 1.63E−07 |
| A029405273 | 1.66E−07 |
| A029405275 | 1.50E−07 |

### 5.20.6 Affinity Determination of the Sequence Optimized Variants

Affinity determination of sequence optimized variants of ISVD A0294041A11 was performed as describe in 6.4, but at 37° C. Results are summarized in Table B-31.

TABLE B-31
Affinity determination of the sequence optimization variants of ISVD A0294041A11

| | Human PD-1 | | | Cyno PD-1 | | |
|---|---|---|---|---|---|---|
| ISVD ID | Kon (1/Ms) | Koff (1/s) | KD (M) | Kon (1/Ms) | Koff (1/s) | KD (M) |
| A0294041A11 | 1.36E+05 | 1.40E−03 | 1.04E−08 | 1.32E+05 | 1.14E−03 | 8.65E−09 |
| A029400075 | 5.90E+05 | 1.10E−03 | 1.80E−09 | 4.60E+05 | 8.70E−04 | 1.90E−09 |
| A029400138 | 1.25E+05 | 1.47E−03 | 1.17E−08 | 6.79E+04 | 1.17E−03 | 1.73E−08 |
| A029400140 | 1.86E+05 | 1.39E−03 | 7.5E−09 | 1.70E+05 | 1.14E−03 | 6.69E−09 |
| A029400146 | 9.78E+04 | 7.11E−03 | 7.27E−08 | 4.09E+04 | 5.88E−03 | 1.44E−07 |
| A029400157 | 3.23E+05 | 1.15E−03 | 3.54E−09 | 3.78E+05 | 9.31E−04 | 2.47E−09 |
| A029400192 | 1.39E+05 | 8.88E−03 | 6.40E−08 | 9.89E+04 | 6.72E−03 | 6.79E−08 |
| A029404018 | 3.20E+05 | 1.13E−03 | 3.55E−09 | 3.62E+05 | 9.11E−04 | 2.52E−09 |
| A029404019 | 3.04E+05 | 1.22E−03 | 4.02E−09 | 3.56E+05 | 9.74E−04 | 2.74E−09 |

### 5.21 Example 12: Sequence Optimization of ISVD A0294040C12

### 5.21.1 Sequence Optimization

ISVD A0294040C12 was further sequence optimized to improve chemical stability, to reduce binding by pre-existing antibodies to the ISVD and to make the ISVD more human like. The optimized sequences are depicted in Tables A-5.

### 5.21.2 Off-Rate Analysis of the Sequence Optimized ISVDs

The sequence optimized ISVDs were subjected to off-rate analysis towards human and cyno PD-1 by Surface Plasmon Resonance (SPR) on a ProteOn XPR36 (Bio-Rad Laboratories), SPR-32 Bruker and an Cytiva Biacore 8K+ instrument. The experiment was performed at 25° C. or 37° C. Thereto, recombinant hPD1::Halotag-histag and cPD-1::Halotag-histag (in house produced) were immobilized on GLC sensorchip (Bio-Rad Laboratories), or a High-Capacity Amine chip (Bruker) or on a CM5 chip (GE Healthcare Life Sciences) via amine coupling, using EDC and NHS chemistry (running buffer used: HBS-EP+, pH7.4). Periplasmic extracts were diluted 1:10 in running buffer (HBS-EP+, pH7.4) and they were injected over the sensor surfaces (association: 120 s, dissociation: 900 s, 45 μL/min). Between sample injections, the surfaces were regenerated with 100 mM HCL (Merck Millipore). Several buffer blanks were injected for double referencing. Data was double referenced by subtracting a reference ligand lane and a blank buffer injection. Processed sensorgrams were analysed based on the 1:1 interaction model (Langmuir binding model) using ProteOn Manager 3.1.0 software (Bio-Rad Laboratories) or using SPR-32 Analyzer 3.2 analysis software or using the Biacore Insight Evaluation software.

The off-rate values for the sequence optimized variants are depicted in Tables B-26 and B-27.

TABLE B-26
Off-rate values for the sequence optimized variants of
ISVD A0294040C12 measured at 25° C.

| | cPD-1 | hPD-1 |
|---|---|---|
| ISVD ID | koff (1/s) | koff (1/s) |
| A0294040C12 | 1.60E−04 | 1.10E−04 |
| A029401738 | 2.30E−03 | 3.10E−03 |
| A029401739 | 6.20E−04 | 7.50E−04 |
| A029401740 | 7.20E−03 | 8.70E−03 |
| A029401741 | 9.40E−02 | 8.30E−02 |
| A029401742 | 3.00E−03 | 3.50E−03 |
| A029401743 | 5.00E−01 | 3.60E−01 |
| A029401744 | 3.00E−03 | 4.00E−03 |
| A029401745 | 2.50E−03 | 3.10E−03 |
| A029401746 | 4.10E−03 | 5.50E−03 |
| A029401747 | 5.00E−01 | 5.00E−01 |
| A029401748 | 4.80E−03 | 6.30E−03 |
| A029401750 | 3.90E−03 | 4.30E−03 |
| A029401755 | 1.40E−03 | 2.00E−03 |
| A029401768 | 1.10E−03 | 1.10E−03 |
| A029401775 | 1.70E−02 | 1.30E−02 |
| A029401776 | 4.00E−03 | 9.00E−03 |
| A029401778 | 1.70E−01 | 2.60E−01 |
| A029401779 | 4.50E−03 | 1.40E−03 |
| A029401780 | 3.10E−03 | 3.70E−03 |
| A029401783 | 1.20E−02 | 6.60E−03 |
| A029401784 | 1.50E−03 | 2.20E−02 |
| A029401785 | 1.50E−02 | 3.70E−03 |
| A029401786 | 2.30E−03 | 3.30E−03 |
| A029401791 | 8.70E−03 | 4.60E−01 |
| A029401794 | 1.60E−03 | 7.90E−03 |
| A029401796 | 5.00E−04 | 8.40E−04 |
| A029401802 | 2.60E−03 | 1.10E−03 |
| A029401806 | 1.90E−02 | 5.30E−03 |
| A029401807 | 1.10E−03 | 1.00E−02 |
| A029401814 | 4.60E−04 | 4.80E−04 |
| A029401815 | 6.20E−04 | 6.80E−04 |
| A029401816 | 1.90E−03 | 6.80E−04 |
| A029401817 | 9.30E−04 | 9.70E−04 |
| A029401818 | 1.40E−03 | 1.60E−01 |
| A029401820 | 1.10E−03 | 1.10E−02 |
| A029401821 | 2.60E−03 | 5.00E−01 |
| A029401822 | 2.00E−03 | 5.00E−01 |
| A029401824 | 2.30E−03 | 3.20E−03 |
| A029401828 | 6.60E−03 | 4.40E−03 |
| A029401830 | 1.00E−03 | 1.10E−03 |
| A029401831 | 5.00E−01 | 2.70E−02 |
| A029401833 | 1.00E−03 | 2.20E−02 |
| A029401834 | 1.10E−03 | 1.50E−03 |
| A029401847 | 1.40E−02 | 9.60E−04 |
| A029401848 | 2.50E−02 | 4.40E−01 |
| A029401852 | 1.30E−03 | 1.40E−03 |
| A029401855 | 1.30E−03 | 9.70E−04 |
| A029401856 | 4.00E−03 | 2.90E−03 |
| A029401857 | 5.00E−01 | 2.00E−03 |
| A029401862 | 1.40E−03 | 5.00E−01 |
| A029401863 | 1.20E−03 | 3.20E−01 |
| A029401866 | 5.00E−01 | 9.00E−04 |
| A029401867 | 0.00E+00 | 1.00E−03 |
| A029401869 | 0.00E+00 | 1.70E−03 |
| A029401871 | 7.80E−04 | 1.20E−03 |
| A029401874 | 7.00E−03 | 5.00E−01 |
| A029401875 | 1.30E−02 | 5.00E−01 |
| A029401881 | 5.00E−01 | 6.30E−03 |
| A029401884 | 2.60E−02 | 2.20E−02 |
| A029401889 | 1.30E−03 | 9.80E−04 |
| A029401891 | 1.10E−03 | 1.00E−03 |
| A029401892 | 8.90E−04 | 7.50E−04 |
| A029401893 | 5.70E−04 | 5.40E−04 |
| A029401894 | 4.10E−04 | 1.70E−03 |
| A029401895 | 6.60E−04 | 5.50E−04 |
| A029401897 | 6.30E−04 | 9.30E−03 |
| A029401898 | 1.10E−03 | 1.00E−03 |
| A029401901 | 4.00E−04 | 4.50E−04 |
| A029401904 | 4.70E−04 | 4.40E−04 |
| A029401906 | 4.10E−04 | 1.00E−03 |
| A029401908 | 5.00E−04 | 6.50E−04 |
| A029401909 | 4.20E−04 | 7.90E−04 |
| A029401922 | 7.40E−04 | 8.30E−04 |
| A029401927 | 1.30E−03 | 9.10E−04 |
| A029401929 | 1.30E−03 | 1.10E−03 |
| A029401930 | 1.90E−03 | 1.10E−03 |
| A029401931 | 4.20E−04 | 7.60E−04 |
| A029401932 | 1.50E−03 | 2.00E−03 |
| A029401934 | 2.00E−03 | 5.00E−01 |
| A029401935 | 1.80E−03 | 5.00E−01 |
| A029401938 | 5.00E−01 | 1.00E−03 |
| A029401939 | 5.00E−01 | 8.70E−04 |
| A029401940 | 5.00E−01 | 1.20E−03 |
| A029401941 | 3.60E−01 | 8.90E−04 |
| A029401942 | 0.00E+00 | 1.30E−03 |
| A029401943 | 1.20E−03 | 1.40E−03 |
| A029401948 | 7.10E−03 | 1.50E−02 |
| A029401953 | 1.10E−02 | 5.00E−01 |
| A029401955 | 4.40E−02 | 5.00E−01 |
| A029401964 | 5.00E−01 | 1.00E−03 |
| A029401965 | 5.00E−01 | 1.10E−03 |
| A029401966 | 2.30E−02 | 9.80E−03 |
| A029401967 | 5.00E−01 | 1.00E−03 |
| A029401968 | 4.20E−01 | 1.00E−03 |
| A029401969 | 5.00E−01 | 9.60E−04 |
| A029401970 | 8.20E−02 | 5.00E−01 |
| A029401971 | 5.00E−01 | 1.10E−03 |
| A029401972 | 8.30E−02 | 1.60E−01 |
| A029401973 | 3.90E−02 | 1.80E−02 |
| A029401981 | 8.40E−02 | 1.90E−01 |
| A029401982 | 1.60E−03 | 5.00E−01 |
| A029401987 | 1.30E−03 | 7.30E−04 |
| A029401991 | 2.00E−01 | 5.00E−01 |
| A029401994 | 5.00E−01 | 5.00E−01 |
| A029402000 | 5.00E−01 | 1.30E−03 |
| A029402002 | 7.30E−04 | 8.20E−04 |
| A029402003 | 5.70E−03 | 7.60E−04 |
| A029402004 | 5.80E−03 | 1.10E−02 |
| A029402006 | 5.00E−01 | 1.20E−03 |
| A029402007 | 4.10E−03 | 1.10E−03 |
| A029402008 | 6.90E−03 | 1.70E−02 |
| A029402010 | 2.90E−03 | 5.90E−03 |
| A029402013 | 7.10E−03 | 1.50E−02 |
| A029402015 | 3.80E−03 | 6.20E−03 |
| A029402017 | 8.70E−03 | 2.00E−03 |
| A029402023 | 4.40E−03 | 6.50E−03 |
| A029402024 | 4.90E−03 | 7.00E−03 |
| A029402025 | 1.30E−03 | 1.50E−02 |
| A029402027 | 1.40E−02 | 5.00E−01 |
| A029402032 | 5.90E−03 | 1.50E−02 |
| A029402037 | 1.40E−02 | 3.60E−03 |
| A029402041 | 6.60E−03 | 1.30E−03 |
| A029402042 | 1.00E−01 | 1.70E−03 |
| A029402043 | 5.50E−03 | 1.10E−03 |
| A029402045 | 5.00E−01 | 2.20E−03 |
| A029402046 | 1.30E−03 | 1.00E−03 |
| A029402047 | 3.70E−03 | 1.50E−03 |
| A029402048 | 3.10E−03 | 1.80E−03 |
| A029402049 | 5.00E−01 | 1.40E−03 |
| A029402050 | 1.60E−03 | 1.40E−03 |
| A029402051 | 5.00E−01 | 1.90E−03 |
| A029402053 | 1.60E−02 | 5.00E−01 |
| A029402056 | 1.90E−02 | 5.00E−01 |
| A029402058 | 5.00E−01 | 5.00E−01 |
| A029402066 | 5.00E−01 | 5.00E−01 |
| A029402073 | 1.70E−03 | 2.10E−03 |
| A029402093 | 1.70E−03 | 1.00E−03 |
| A029402104 | 2.50E−02 | 5.00E−01 |
| A029403783 | 8.50E−03 | nd |
| A029403787 | 3.60E−03 | 1.50E−03 |
| A029403788 | 5.00E−03 | 1.70E−03 |
| A029403789 | 4.30E−03 | 1.40E−03 |
| A029403791 | 9.00E−04 | 6.70E−04 |
| A029403792 | 2.70E−03 | 1.80E−03 |
| A029403793 | 1.40E−03 | 6.50E−03 |
| A029403796 | 0.00E+00 | 2.30E−03 |
| A029403798 | 0.00E+00 | 1.30E−02 |
| A029403801 | 1.30E−02 | 1.50E−03 |
| A029403803 | 2.70E−03 | 1.90E−03 |
| A029403805 | 7.20E−03 | 1.10E−02 |
| A029403806 | 4.40E−02 | 3.00E−03 |
| A029403807 | 0.00E+00 | 2.60E−03 |
| A029403810 | 4.80E−03 | 2.80E−03 |
| A029403812 | 1.60E−03 | 1.10E−03 |
| A029403813 | 2.20E−03 | 1.20E−03 |
| A029403814 | 2.40E−03 | 1.40E−03 |
| A029403827 | 0.00E+00 | 3.10E−03 |
| A029403830 | 1.90E−03 | 9.70E−04 |
| A029403842 | 3.00E−03 | 1.10E−03 |
| A029403844 | 3.90E−03 | 1.50E−03 |
| A029403855 | 0.00E+00 | 2.90E−03 |
| A029403857 | 4.50E−03 | 1.70E−03 |

TABLE B-27
Off-rate values for the sequence optimized variants
of A0294040C12 measured at 37° C.

| | cPD-1 | hPD-1 |
|---|---|---|
| ISVD ID | koff (1/s) | koff (1/s) |
| A0294040C12 | 1.16E−04 | 1.16E−04 |
| A029400112 | 1.53E−04 | 1.45E−04 |
| A029400113 | 2.05E−04 | 2.14E−04 |
| A029400114 | 1.41E−04 | 1.12E−04 |
| A029400115 | 2.13E−04 | 1.41E−04 |
| A029400116 | 7.70E−04 | 9.28E−04 |
| A029400117 | 1.28E−04 | 8.12E−05 |
| A029400118 | 1.08E−03 | 1.30E−03 |
| A029400119 | 1.46E−04 | 1.26E−04 |
| A029400120 | 1.23E−04 | 9.21E−05 |
| A029400121 | 1.72E−04 | 1.32E−04 |
| A029400122 | 1.94E−04 | 1.97E−04 |
| A029400123 | 7.56E−04 | 9.40E−04 |
| A029400124 | 1.97E−04 | 1.95E−04 |
| A029400125 | 3.59E−04 | 2.74E−04 |
| A029400126 | 9.05E−05 | 6.94E−05 |
| A029400127 | 1.83E−04 | 1.94E−04 |
| A029400128 | 1.99E−04 | 2.11E−04 |
| A029400129 | 2.07E−04 | 2.22E−04 |
| A029400130 | 1.62E−04 | 1.17E−04 |
| A029400131 | 2.05E−04 | 2.00E−04 |
| A029400132 | 9.64E−05 | 8.37E−05 |
| A029400133 | 1.88E−04 | 1.88E−04 |
| A029400134 | 2.51E−04 | 2.91E−04 |
| A029400179 | 1.25E−04 | 1.28E−04 |
| A029400180 | 1.06E−03 | 1.27E−03 |
| A029400181 | 1.46E−04 | 1.01E−04 |
| A029400182 | 7.05E−04 | 8.27E−04 |
| A029400183 | 2.29E−04 | 1.49E−04 |
| A029400184 | 1.43E−04 | 7.80E−05 |
| A029400185 | 1.88E−04 | 9.41E−05 |
| A029400186 | 1.48E−04 | 1.45E−04 |
| A029400187 | 1.60E−04 | 1.32E−04 |
| A029400188 | 5.88E−04 | 6.35E−04 |
| A029400189 | 8.11E−04 | 9.24E−04 |
| A029400190 | 5.81E−04 | 6.20E−04 |
| A029400198 | 1.50E−04 | 1.30E−04 |
| A029404626 | 2.35E−03 | 2.10E−03 |
| A029404628 | 2.12E−03 | 2.12E−03 |
| A029404638 | 8.85E−04 | 1.16E−03 |
| A029404639 | 9.90E−04 | 1.35E−03 |
| A029404640 | 9.61E−04 | 1.29E−03 |
| A029404641 | 2.94E−03 | 4.12E−03 |
| A029404642 | 8.99E−03 | 1.20E−02 |
| A029404643 | 1.01E−02 | 1.39E−02 |
| A029404644 | 9.80E−03 | 1.34E−02 |
| A029404645 | 2.04E−02 | 2.55E−02 |
| A029404650 | 2.43E−02 | 3.22E−02 |
| A029404651 | 1.56E−02 | 1.89E−02 |
| A029404652 | 2.12E−02 | 2.58E−02 |
| A029404653 | 1.87E−02 | 2.03E−02 |
| A029404656 | 6.24E−03 | 6.59E−03 |
| A029404657 | 7.30E−03 | 8.57E−03 |
| A029404658 | 7.13E−03 | 7.65E−03 |
| A029404660 | 2.57E−02 | 2.60E−02 |
| A029404662 | 2.08E−02 | 2.69E−02 |
| A029404663 | 2.97E−04 | 3.78E−04 |
| A029404664 | 2.85E−02 | 3.19E−02 |
| A029404665 | 2.73E−04 | 3.44E−04 |
| A029404667 | 7.89E−04 | 9.56E−04 |
| A029404668 | 3.74E−04 | 4.68E−04 |
| A029404669 | 5.23E−04 | 6.77E−04 |
| A029404670 | 5.18E−04 | 6.06E−04 |
| A029404671 | 8.23E−04 | 1.03E−03 |
| A029404672 | 1.99E−03 | 2.74E−03 |
| A029404673 | 2.75E−03 | 4.05E−03 |
| A029404674 | 2.33E−03 | 3.58E−03 |
| A029404675 | 6.95E−03 | 8.53E−03 |
| A029404809 | 4.18E−04 | 5.57E−04 |
| A029404834 | 4.89E−03 | 6.67E−03 |
| A029404835 | 1.08E−02 | 1.30E−02 |
| A029404836 | 1.19E−03 | 1.52E−03 |
| A029404837 | 2.18E−03 | 2.66E−03 |
| A029404838 | 1.22E−03 | 1.75E−03 |
| A029404839 | 2.95E−03 | 4.31E−03 |
| A029404840 | 2.01E−03 | 2.80E−03 |
| A029404841 | 3.39E−03 | 4.32E−03 |
| A029404843 | 6.34E−03 | 1.06E−02 |
| A029404844 | 3.51E−03 | 4.95E−03 |
| A029404859 | 1.46E−04 | 2.11E−04 |
| A029404861 | 1.72E−04 | 2.43E−04 |
| A029404862 | 4.24E−04 | 6.24E−04 |
| A029404863 | 8.18E−03 | 1.08E−02 |
| A029404867 | 3.55E−03 | 3.86E−03 |
| A029405022 | 4.44E−03 | 5.83E−03 |
| A029405023 | 1.41E−02 | 1.19E−02 |

### 5.21.3 Biophysical Characterization of the Sequence Optimized ISVDs

The biophysical characteristics of the sequence optimized ISVDs of ISVD A0294040C12 were tested in thermal shift assay (Tm) and a temperature of onset of aggregation assay (Tagg).

The thermal shift assay and Tagg assay were performed as described in example 6.5.1 and 6.5.4 respectively. The Tm and Tagg values obtained for the sequence optimized variants are shown in Table B-28.

TABLE B-28
Biophysical properties of the sequence optimized
variants of ISVD A0294040C12

| ISVD ID | Tm (° C.) | Tagg (° C.) |
|---|---|---|
| A0294040C12 | 68.6 | nd |
| A029400114 | 64.5 | 63.2 |
| A029400121 | 69.2 | 66.5 |
| A029400125 | 55.9 | 53.2 |
| A029400126 | 72.4 | nd |
| A029400134 | 68.6 | nd |
| A029400181 | 60 | 58 |
| A029400184 | 68.9 | 66.2 |
| A029400185 | 62.9 | 63.2 |
| A029400186 | 63.3 | 63.7 |
| A029400198 | 70 | nd |
| A029403745 | 75 | nd |
| A029403746 | 72 | nd |
| A029403747 | 66 | nd |
| A029404020 | 49.6 | nd |
| A029404023 | 54.5 | nd |
| A029404024 | 54.5 | nd |
| A029404025 | 61.8 | nd |
| A029404027 | 50.1 | nd |
| A029404661 | 76.8 | nd |
| A029404809 | 66.1 | nd |
| A029404836 | 60.8 | nd |
| A029404859 | 72.0 | nd |
| A029404861 | 55.4 | nd |
| A029404862 | 49.1 | nd |

nd: not determined

### 5.21.4 Characterization of the Sequence Optimized ISVDs for Binding Cells Expressing PD-1

The sequence optimized variants of ISVD A0294040C12 were tested for FACS binding on CHO cell lines and PreB 300-19 cells expressing human or cyno PD-1 (in house made). The binding FACS was carried out as described in Example 6.1.3. Results are shown in Tables B-29 and B-30.

TABLE B-29
FACS binding of the sequence optimized variants of ISVD
A0294040C12 on CHO cells expressing human or cyno PD-1

| | cyno PD-1 | human PD-1 |
|---|---|---|
| ISVD ID | EC50 (M) | EC50 (M) |
| A0294040C12 | 1.24E−08 | 4.30E−08 |
| A029403745 | 3.22E−10 | 3.52E−10 |
| A029404661 | 4.15E−10 | 3.65E−10 |

TABLE B-30
FACS binding of the sequence optimized variants of ISVD
A0294040C12 on PreB 300-19 cells expressing human or cyno PD-1

| ISVD ID | cyno PD-1 EC50 | human PD-1 EC50 |
|---|---|---|
| A029403745 | 3.37E−09 | 1.13E−08 |
| A029404661 | 3.34E−09 | 1.16E−08 |

### 5.21.5 Affinity Determination of the Sequence Optimized Variants

Affinity determination of sequence optimized variants of ISVD A0294040C12 was performed as describe in 6.4, but at 37° C. Results are summarized in Table B-32.

TABLE B-32
Affinity determination of the sequence optimization variants of ISVD A0294040C12

| | Human PD-1 | | | Cyno PD-1 | | |
|---|---|---|---|---|---|---|
| ISVD ID | Kon (1/Ms) | Koff (1/s) | KD (M) | Kon (1/Ms) | Koff (1/s) | KD (M) |
| A0294040C12 | 1.22E+05 | 3.19E−04 | 2.61E−09 | 1.10E+05 | 2.14E−04 | 1.94E−09 |
| A029400184 | 1.32E+05 | 3.57E−04 | 2.71E−09 | 1.11E+05 | 2.54E−04 | 2.29E−09 |
| A029403745 | 7.94E+04 | 1.54E−03 | 1.94E−08 | 7.28E+04 | 1.12E−03 | 1.54E−08 |
| A029403746 | 9.31E+04 | 1.47E−03 | 1.58E−08 | 8.45E+04 | 1.07E−03 | 1.26E−08 |
| A029403747 | 9.40E+04 | 2.75E−03 | 2.93E−08 | 8.31E+04 | 1.90E−03 | 2.29E−08 |

### 5.22 Example 13: Structure Determination of Anti-PD-1 ISVDs Interacting with PD-1

Crystallization was applied to determine the epitope on PD-1 bound by the anti-PD-1 ISVDs and the paratope on these anti-PD-1 ISVDs.

The recombinant human PD-1 extracellular domain (residues 34-150; UniProt: Q15116) with C93S mutation was purchase from Recepton and expressed in E. coli in the form of inclusion bodies and purified, as described previously (Yin-wei Lin et al. 2008, PNAS 105:3011-3016). The VHHs were produced as described in Example 3. The purified proteins, VHHs and hPD-1 extracellular domain were mixed at a molar ratio of 1.5:1 and incubated at 25° C. for 20 min and then loaded onto gel filtration to obtain homogenous VHH/PD-1 complex.

### 6.13.1 Determination of the A0294047E02/PD-1 Interaction Site

### Materials and Methods

The protein complex A0294047E02/PD-1 was concentrated to 10.6 mg/ml in PBS1× buffer. Crystals were obtained by vapor diffusion experiments in hanging drop geometry under 30% PEG3350, 50 mM TRIS, pH 8 at 19° C.

X-ray diffraction data set was collected at ESRF synchrotron beamline ID30A-1 and processed using the Global Phasing Suite (Vonrhein C, Flensburg C, Keller P, et al. Data processing and analysis with the autoPROC toolbox. Acta Crystallogr D Biol Crystallogr. 2011; 67:293-302.) and CCP4 (Winn M D, Ballard C C, Cowtan K D, et al. Overview of the CCP4 suite and current developments. ActaCrystallogr D Biol Crystallogr. 2011; 67:235-242.) software. The structure was determined by molecular replacement using Phaser (McCoy A J, Grosse-Kuntsleve R W, Adams P D, et al. Phaser crystallographic software. J Appl Crystallogr. 2007; 40:658-674.) with in-house PDB models of A0294047E02 and PD-1. Refinement was carried out by successive rounds of Buster (Blanc E, Roversi P, Vonrhein C, et al. Refinement of severely incomplete structures with maximum likelihood in BUSTER-TNT. Acta Crystallogr D BiolCrystallogr. 2004; 60:2210-2221.) and COOT (Emsley P, Lohkamp B, Scott W G, et al. Features and development of Coot. Acta Crystallogr D Biol Crystallogr.2010; 66:486-501.). The Crystal space group is P21 with 2 complexes per asymmetric unit and final refinement statistics are Rfree=23.9%/Rwork=20.5% at 1.78 Å resolution.

### Structure of the Complex

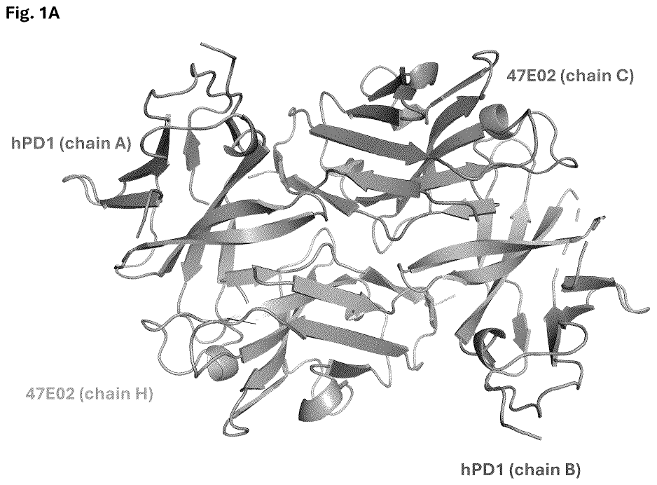

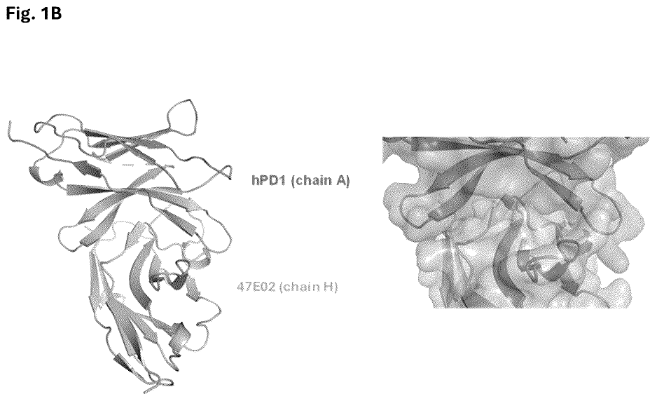

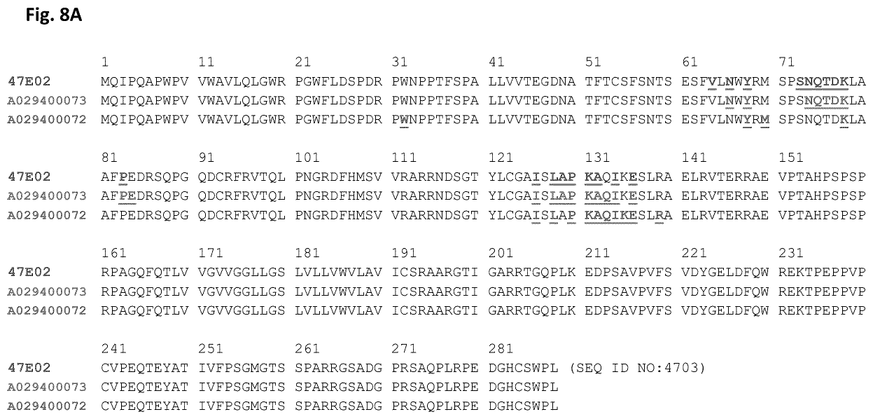

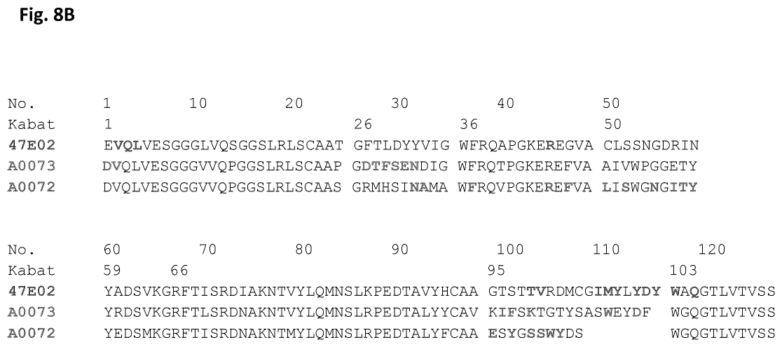

A view of the structure of the interaction of A0294047E02 with PD-1 is given in FIG. 1A. A0294047E02 forms 2 complexes with PD-1 spanning an interface on PD-1 of about 778 Å² (chain A) and 748 Å² (chain C) respectively. Analysis of the epitope-paratope interface (FIG. 2) showed involvement in binding by following amino acids (interacting residues with a distance of <4.0 Å):
PD-1 epitope (FIGS. 2B and 8A): V64, N66, Y68, S73, N74, Q75, T76, D77, K78, P83, 1126, L128, A129, P130, K131, A132, I134, E136
A0294047E02 paratope (FIGS. 2A and 8B): positions V2, Q3, L4, R45, T103, V104, I110, M111, Y112, Y114, D115, Y116, W117, Q119, which is positions V2, Q3, L4, R45, T99, V100, I100e, M100g, Y100h, Y100j, D101, Y102, W103, Q105, according to Kabat numbering

11 hydrogen bonds were observed distributed across the first interface (A0294047E02 (H chain) with PD-1 (A chain)):

| ISVD A0294047E02 (H chain) | | Length | Amino acid in |
|---|---|---|---|
| Aa residue | Kabat numbering | (Å) | PD-1 (A chain) |
| Q3 [NE2] | Q3 [NE2] | 3.9 Å | S73 [OG] |
| Q3 [NE2] | Q3 [NE2] | 3.9 Å | Q75 [OE1] |
| L4 [N] | L4 [N] | 2.9 Å | Q75 [OE1] |
| Y114 [OH] | Y100j [OH] | 3.7 Å | A132 [O] |
| Y114 [O] | Y100j [O] | 3.1 Å | N66 [ND2] |
| D115 [OD1] | D101 [OD1] | 3.0 Å | N66 [ND2] |
| Y112 [OH] | Y100h [OH] | 3.5 Å | N66 [ND2] |
| Y114 [O] | Y100j [O] | 2.5 Å | Y68 [OH] |
| V2 [O] | V2 [O] | 2.8 Å | Q75 [NE2] |
| D115 [O] | D101 [O] | 3.0 Å | K78 [N] |
| I110 [O] | I100e [O] | 2.8 Å | A132 [N] |

N: backbone amide nitrogen (—NH);
ND2: terminal nitrogen in the side-chain amide group of asparagine (N);
NE2: terminal nitrogen in amide of glutamine (Q) or imidazole ring of histidine (H);
O: backbone carbonyl oxygen (C═O);
OD1: One of the two carboxylate oxygen atoms in the acidic side chain of aspartate (D);
OE1: one of the two carboxylate oxygen atoms in the side chain of glutamate (E);
OG: hydroxyl oxygen atom in the polar side chain of serine (S);
OH: hydroxyl group in tyrosine (Y).

Three salt bridges were distributed across the first interface (A0294047E02 (H chain) with PD-1 (A chain)), between acidic residues R45 of ISVD A0294047E02 (R45 according to Kabat numbering) and E136 of PD-1:

| ISVD A0294047E02 (H chain) | | Length | Amino acid in |
|---|---|---|---|
| Aa residue | Kabat numbering | (Å) | PD-1 (A chain) |
| R45 [NH2] | R45 [NH2] | 3.9 Å | E136 [OE1] |
| R45 [NH2] | R45 [NH2] | 2.7 Å | E136 [OE2] |
| R45 [NH1] | R45 [NH1] | 2.8 Å | E136 [OE2] |

NH1: one of the terminal nitrogen atoms in the guanidinium group of arginine (R);
NH2: the other terminal nitrogen in the guanidinium group of arginine;
OE1: one of the two carboxylate oxygen atoms in the side chain of glutamate (E);
OE2: the second carboxylate oxygen atom in the side chain of glutamate (E).

Eleven hydrogen bonds were observed distributed across the second interface (A0294047E02 (C chain) with PD-1 (B-chain)):

| ISVD A0294047E02 (C chain) | | | |
|---|---|---|---|
| Aa residue | Kabat numbering | Length (Å) | Amino acid in PD-1 (B chain) |
| Q3 [NE2] | Q3 [NE2] | 3.9 Å | S73 [OG] |
| L4 [N] | L4 [N] | 2.9 Å | Q75 [OE1] |
| Y114 [OH] | Y100j [OH] | 3.8 Å | A132 [O] |
| Y112 [OH] | Y100h [OH] | 3.4 Å | N66 [ND2] |
| Y114 [O] | Y100j [O] | 3.2 Å | N66 [ND2] |
| D115 [OD1] | D101 [OD1] | 3.0 Å | N66 [ND2] |
| Y114 [O] | Y100j [O] | 2.5 Å | Y68 [OH] |
| Q119 [OE1] | Q105 [OE1] | 2.9 Å | N74 [ND2] |
| V2 [O] | V2 [O] | 2.9 Å | Q75 [NE2] |
| D115 [O] | D101 [O] | 2.9 Å | K78 [N] |
| I110 [O] | I100e [O] | 2.9 Å | A132 [N] |

N: backbone amide nitrogen (—NH);
ND2: terminal nitrogen in the side-chain amide group of asparagine (N);
NE2: terminal nitrogen in amide of glutamine (Q) or imidazole ring of histidine (H);
O: backbone carbonyl oxygen (C═O);
OD1: One of the two carboxylate oxygen atoms in the acidic side chain of aspartate (D);
OD2: the second carboxylate oxygen atom in the side chain of aspartate (D);
OE1: one of the two carboxylate oxygen atoms in the side chain of glutamate (E);
OG: hydroxyl oxygen atom in the polar side chain of serine (S);
[OH]: hydroxyl group in tyrosine (Y);
OH: hydroxyl group in tyrosine (Y).

Two salt bridges were distributed across the second interface (A0294047E02 (C chain) with PD-1 (B-chain)), between acidic residues R45 of ISVD A0294047E02 (R45 according to Kabat numbering) and E136 of PD-1 (FIG. 2):

ISVD A0294047E02 (C chain)

| Aa | Kabat | Length | Amino acid in |
|---|---|---|---|
| residue | numbering | (Å) | PD-1 (B chain) |
| R45 [NH1] | R45 [NH1] | 2.9 Å | E136 [OE2] |
| R45 [NH2] | R45 [NH2] | 2.9 Å | E136 [OE2] |

NH1: one of the terminal nitrogen atoms in the guanidinium group of arginine (R); NH2: the other terminal nitrogen in the guanidinium group of arginine; OE2: the second carboxylate oxygen atom in the side chain of glutamate (E).

CDR3 residues form the core of interface with hPD-1, and there are multiple interactions of CDR3 residues with hPD-1. The crystal structure shows a B-sheet formed by the interactions of CDR3 with a B-strand from hPD-1 (FIG. 1B).

### 6.13.2 Determination of the A029400073 (A0294041A11 Family Member)/PD-1 Interaction Site

### Materials and Methods

The protein complex A029400073/PD-1 was concentrated to 8 mg/ml in PBS1× buffer. Crystals were obtained by vapor diffusion experiments in hanging drop geometry under 100 mM Na Cacodylate, 300 mM Na Acetate, 10% PEG1000, pH 6.5 at 19° C. X-ray diffraction data set was collected at SOLEIL synchrotron beamline PX-1 and processed using the Global Phasing Suite (Vonrhein C, Flensburg C, Keller P, et al. Data processing and analysis with the autoPROC toolbox. Acta Crystallogr D Biol Crystallogr. 2011; 67:293-302.) and CCP4 (Winn M D, Ballard C C, Cowtan K D, et al. Overview of the CCP4 suite and current developments. ActaCrystallogr D Biol Crystallogr. 2011; 67:235-242.) software. The structure was determined by molecular replacement using Phaser (McCoy A J, Grosse-Kuntsleve R W, Adams P D, et al. Phaser crystallographic software. J Appl Crystallogr. 2007; 40:658-674.) with in-house PDB models of A029400073 and PD-1. Refinement was carried out by successive rounds of Buster (Blanc E, Roversi P, Vonrhein C, et al. Refinement of severely incomplete structures with maximum likelihood in BUSTER-TNT. Acta Crystallogr D BiolCrystallogr. 2004; 60:2210-2221.) and COOT (Emsley P, Lohkamp B, Scott W G, et al. Features and development of Coot. Acta Crystallogr D Biol Crystallogr. 2010; 66:486-501.). The Crystal space group is P1 with 4 complexes per asymmetric unit and final refinement statistics are Rfree=26.7%/Rwork=22.3% at 2.36 Å resolution.

### Structure of the Complex

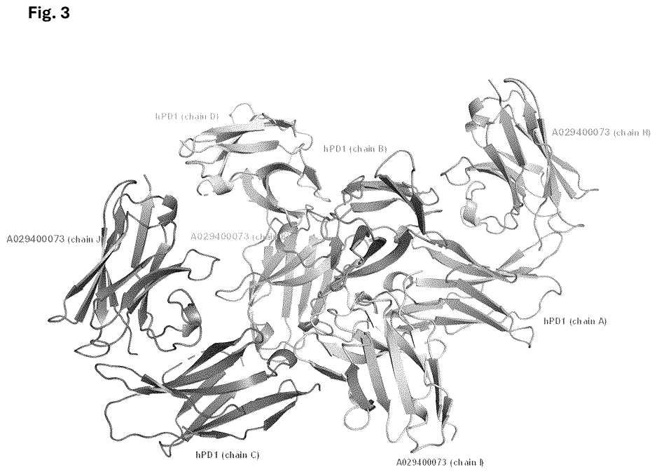

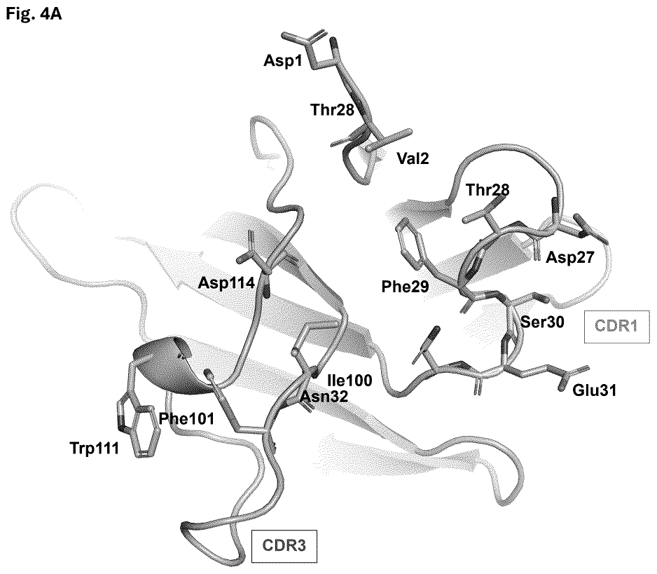

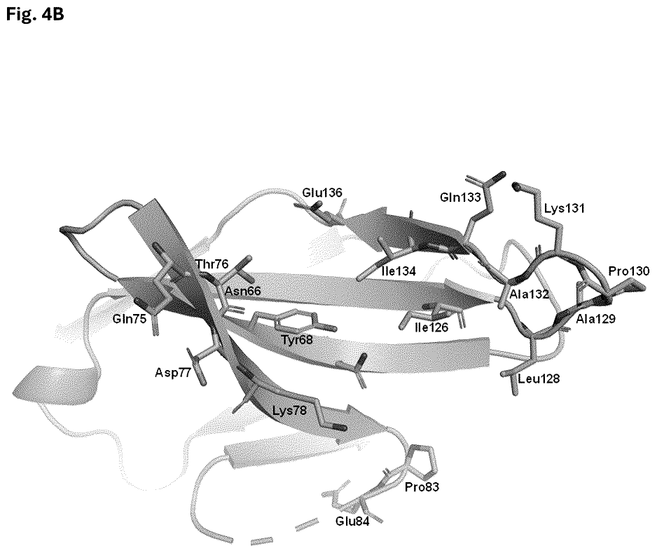

A view of the structure of the interaction of A029400073 with PD-1 is given in FIG. 3. A029400073 forms 4 complexes with PD-1 spanning an interface on PD-1 of between 624 Å² and 703 Å2. Analysis of the epitope-paratope interface (FIG. 4) showed involvement in binding by following amino acids (interacting residues with a distance of <4.0 Å):
PD-1 epitope (FIGS. 4B and 8A): N66, Y68, N74, Q75, T76, D77, K78, P83, E84, I126, L128, A129, P130, K131, A132, Q133, I134, E136
A029400073 paratope (FIGS. 4A and 8B): D1, V2, D27, T28, F29, S30, E31, N32, 1100, F101, K103, W111, D114, which is positions D1, V2, D27, T28, F29, S30, E31, N32, 196, F97, K99, W100g, D101 according to Kabat numbering.

Six hydrogen bonds were observed distributed across the first interface (A029400073 (H chain) with PD-1 (A chain)):

ISVD A029400073 (H chain)

| Aa | Kabat | Length | Amino acid in |
|---|---|---|---|
| residue | numbering | (Å) | PD-1 (A chain) |
| F29 [N] | F29 [N] | 3.2 Å | A129 [O] |
| S30 [N] | S30 [N] | 3.3 Å | P130 [O] |
| E31 [N] | E31 [N] | 2.9 Å | P130 [O] |
| N32 [ND2] | N32 [ND2] | 3.3 Å | P130 [O] |
| D114 [OD2] | D101 [OD2] | 3.8 Å | N66 [ND2] |
| E31 [O] | E31 [O] | 3.5 Å | K131 [NZ] |

N: backbone amide nitrogen (—NH); ND2: terminal nitrogen in the side-chain amide group of asparagine (N); NZ: terminal amino group nitrogen in the basic side chain of lysine (K); O: backbone carbonyl oxygen (C═O); OD2: the second carboxylate oxygen atom in the side chain of aspartate (D).

Two salt bridges were distributed across the first interface (A029400073 (H chain) with PD-1 (A chain)):

ISVD A029400073 (H chain)

| Aa | Kabat | Length | Amino acid in |
|---|---|---|---|
| residue | numbering | (Å) | PD-1 (A chain) |
| D114 [OD2] | D101 [OD2] | 3.5 Å | K78 [NZ] |
| D114 [OD1] | D101 [OD1] | 3.0 Å | K78 [NZ] |

NZ: terminal amino group nitrogen in the basic side chain of lysine (K); OD1: One of the two carboxylate oxygen atoms in the acidic side chain of aspartate (D); OD2: the second carboxylate oxygen atom in the side chain of aspartate (D).

Six hydrogen bonds were observed distributed across the second interface (A029400073 (I chain) with PD-1 (B chain)):

ISVD A029400073 (I chain)

| Aa | Kabat | Length | Amino acid in |
|---|---|---|---|
| residue | numbering | (Å) | PD-1 (B chain) |
| F29 [N] | F29 [N] | 3.2 Å | A129 [O] |
| S30 [N] | S30 [N] | 3.3 Å | P130 [O] |
| E31 [N] | E31 [N] | 2.9 Å | P130 [O] |
| N32 [ND2] | N32 [ND2] | 3.3 Å | P130 [O] |
| E31 [O] | E31 [O] | 3.2 Å | K131 [NZ] |
| D114 [OD1] | D101 [OD1] | 3.7 Å | N66 [ND2] |

N: backbone amide nitrogen (—NH); ND2: terminal nitrogen in the side-chain amide group of asparagine (N); NZ: terminal amino group nitrogen in the basic side chain of lysine (K); O: backbone carbonyl oxygen (C═O); OD1: One of the two carboxylate oxygen atoms in the acidic side chain of aspartate (D).

Two salt bridges were distributed across the second interface (A029400073 (I chain) with PD-1 (B chain)):

ISVD A029400073 (I chain)

| Aa | Kabat | Length | Amino acid in |
|---|---|---|---|
| residue | numbering | (Å) | PD-1 (B chain) |
| D114 [OD1] | D101 [OD1] | 3.4 Å | K78 [NZ] |
| D114 [OD2] | D101 [OD2] | 3.3 Å | K78 [NZ] |

NZ: terminal amino group nitrogen in the basic side chain of lysine (K); OD1: One of the two carboxylate oxygen atoms in the acidic side chain of aspartate (D); OD2: the second carboxylate oxygen atom in the side chain of aspartate (D).

Seven hydrogen bonds were observed distributed across the third interface (A029400073 (J chain) with PD-1 (C chain)):

ISVD A029400073 (J chain)

| Aa | Kabat | Length | Amino acid in |
|---|---|---|---|
| residue | numbering | (Å) | PD-1 (C chain) |
| F29 [N] | F29 [N] | 3.1 Å | A129 [O] |
| S30 [N] | S30 [N] | 3.2 Å | P130 [O] |
| E31 [N] | E31 [N] | 2.8 Å | P130 [O] |
| N32 [ND2] | N32 [ND2] | 3.5 Å | P130 [O] |
| K103 [NZ] | K99 [NZ] | 3.3 Å | N74 [O] |
| D114 [OD2] | D101 [OD2] | 3.7 Å | N66 [ND2] |
| E31 [O] | E31 [O] | 3.6 Å | K131 [NZ] |

N: backbone amide nitrogen (—NH); ND2: terminal nitrogen in the side-chain amide group of asparagine (N); NZ: terminal amino group nitrogen in the basic side chain of lysine (K); O: backbone carbonyl oxygen (C═O); OD2: the second carboxylate oxygen atom in the side chain of aspartate (D).

Two salt bridges were distributed across the third interface (A029400073 (J chain) with PD-1 (C chain)):

ISVD A029400073 (J chain)

| Aa | Kabat | Length | Amino acid in |
|---|---|---|---|
| residue | numbering | (Å) | PD-1 (C chain) |
| D114 [OD1] | D101 [OD1] | 3.2 Å | K78 [NZ] |
| D114 [OD2] | D101 [OD2] | 3.2 Å | K78 [NZ] |

NZ: terminal amino group nitrogen in the basic side chain of lysine (K); OD1: One of the two carboxylate oxygen atoms in the acidic side chain of aspartate (D); OD2: the second carboxylate oxygen atom in the side chain of aspartate (D).

Six hydrogen bonds were observed distributed across the fourth interface (A029400073 (K chain) with PD-1 (D chain)):

ISVD A029400073 (K chain)

| Aa | Kabat | Length | Amino acid in |
|---|---|---|---|
| residue | numbering | (Å) | PD-1 (D chain) |
| F29 [N] | F29 [N] | 3.1 Å | A129 [O] |
| S30 [N] | S30 [N] | 3.3 Å | P130 [O] |
| E31 [N] | E31 [N] | 2.8 Å | P130 [O] |
| N32 [ND2] | N32 [ND2] | 3.4 Å | P130 [O] |
| K103 [O] | K99 [O] | 3.8 Å | N74 [ND2] |
| D114 [OD1] | D101 [OD1] | 3.2 Å | N66 [ND2] |

N: backbone amide nitrogen (—NH); ND2: terminal nitrogen in the side-chain amide group of asparagine (N); NZ: terminal amino group nitrogen in the basic side chain of lysine (K); O: backbone carbonyl oxygen (C═O); OD1: One of the two carboxylate oxygen atoms in the acidic side chain of aspartate (D).

Four salt bridges were distributed across the fourth interface (A029400073 (K chain) with PD-1 (D chain)):

ISVD A029400073 (K chain)

| Aa | Kabat | Length | Amino acid in |
|---|---|---|---|
| residue | numbering | (Å) | PD-1 (D chain) |
| K103 [NZ] | K99 [NZ] | 3.3 Å | E136 [OE1] |
| K103 [NZ] | K99 [NZ] | 3.7 Å | E136 [OE2] |
| D114 [OD1] | D101 [OD1] | 3.6 Å | K78 [NZ] |
| D114 [OD2] | D101 [OD2] | 3.1 Å | K78 [NZ] |

NZ: terminal amino group nitrogen in the basic side chain of lysine (K); OD1: One of the two carboxylate oxygen atoms in the acidic side chain of aspartate (D); OD2: the second carboxylate oxygen atom in the side chain of aspartate (D); OE1: one of the two carboxylate oxygen atoms in the side chain of glutamate (E); OE2: the second carboxylate oxygen atom in the side chain of glutamate (E).

The interface is principally built upon CDR1 and CDR3 regions.

### 6.13.3 Determination of the A029400072 (A0294037F03 Family Member)/PD-1 Interaction Site

### Material and Methods

The protein complex A029400072/PD-1 was concentrated to 12.0 mg/mL in PBS1× buffer. Crystals were obtained by vapor diffusion experiments in hanging drop geometry under 1 M Na Acetate, 1M (NH₄)₂HPO₄, pH 4.5 at 19° C.

X-ray diffraction data set was collected at ESRF synchrotron beamline ID30A-1 and processed using the Global Phasing Suite (Vonrhein C, Flensburg C, Keller P, et al. Data processing and analysis with the autoPROC toolbox. Acta Crystallogr D Biol Crystallogr. 2011; 67:293-302.) and CCP4 (Winn M D, Ballard C C, Cowtan K D, et al. Overview of the CCP4 suite and current developments. ActaCrystallogr D Biol Crystallogr. 2011; 67:235-242.) software. The structure was determined by molecular replacement using Phaser (McCoy A J, Grosse-Kuntsleve R W, Adams P D, et al. Phaser crystallographic software. J Appl Crystallogr. 2007; 40:658-674.) with in-house PDB models of A0294041A11 and PD-1. Refinement was carried out by successive rounds of Buster (Blanc E, Roversi P, Vonrhein C, et al. Refinement of severely incomplete structures with maximum likelihood in BUSTER-TNT. Acta Crystallogr D BiolCrystallogr. 2004; 60:2210-2221.) and COOT (Emsley P, Lohkamp B, Scott W G, et al. Features and development of Coot. Acta Crystallogr D Biol Crystallogr. 2010; 66:486-501.). The Crystal space group is P41212 with 2 complexes per asymmetric unit and final refinement statistics are Rfree=23.9%/Rwork=20.3% at 3.12 Å resolution.

### Structure of the Complex

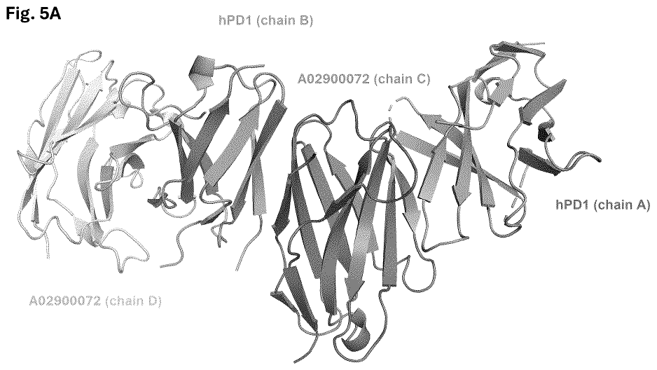

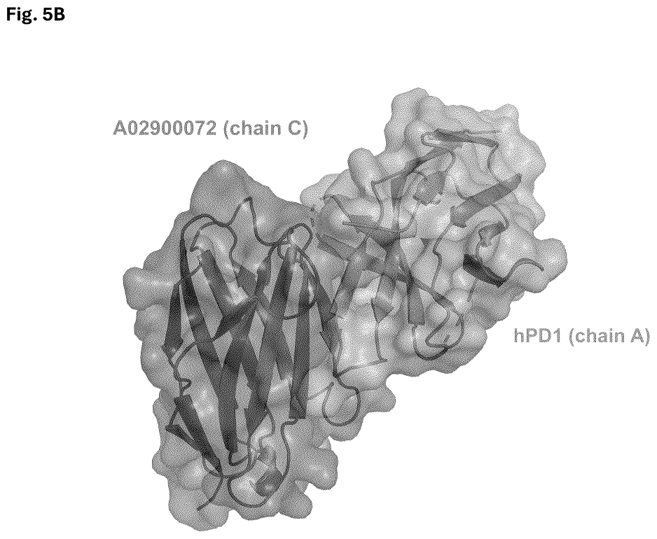

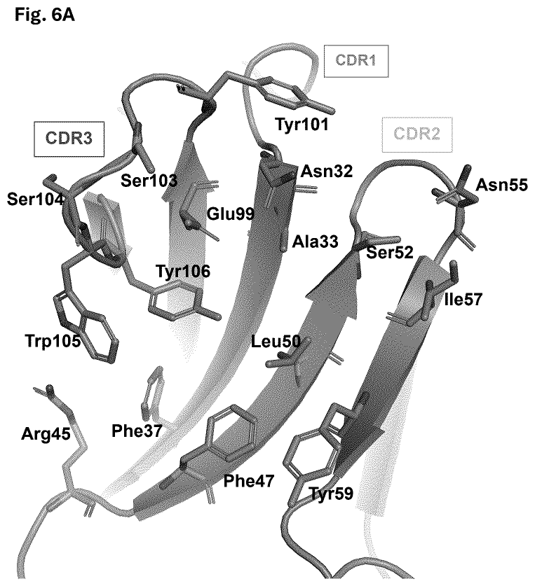

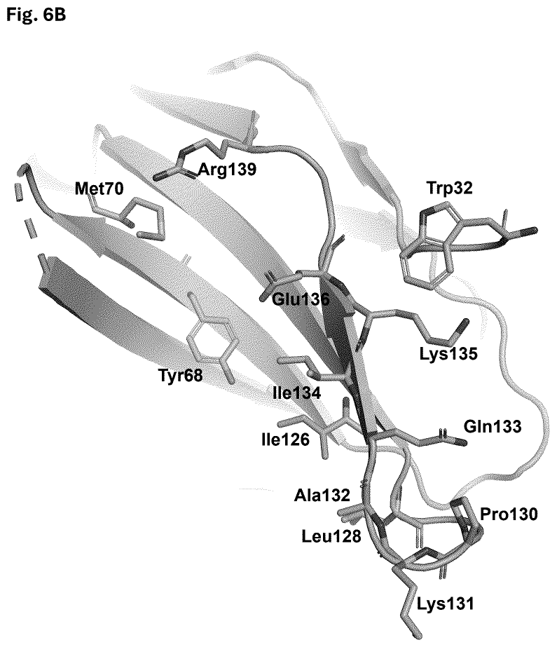

A view of the structure of the interaction of A029400072 with PD-1 is given in FIG. 5. A029400072 forms 2 complexes with PD-1 spanning an interface on PD-1 of about 735 Å² and 762 Å² respectively. Analysis of the epitope-paratope interface (FIG. 6) showed involvement in binding by following amino acids (interacting residues with a distance of <4.0 Å):
PD-1 epitope (FIGS. 6B and 8A): W32, Y68, M70, K78, I126, L128, P130, K131, A132, Q133, I134, K135, E136, R139.
A029400072 paratope (FIGS. 6A and 8B): positions N32, A33, F37, R45, F47, L50, S52, N55, 157, T58, Y59, E99, Y101, S103, S104, W105, Y106, which are positions N32, A33, F37, R45, F47, L50, S52, N54, 156, T57, Y58, E95, Y97, S99, S100, W100a, Y100b according to Kabat numbering.

Three hydrogen bonds were observed distributed across the first interface (A029400072 (C chain) with PD-1 (A chain)) (FIG. 6):

ISVD A029400072 (C chain)

| Aa | Kabat | Length | Amino acid in |
|---|---|---|---|
| residue | numbering | (Å) | PD-1 (A chain) |
| Y106 [OH] | Y100b [OH] | 3.0 Å | A132 [O] |
| S104 [O] | S100 [O] | 3.8 Å | Q133 [NE2] |
| S103 [OG] | S99 [OG] | 2.9 Å | K135 [NZ] |

NE2: terminal nitrogen in amide of glutamine (Q) or imidazole ring of histidine (H); NZ: terminal amino group nitrogen in the basic side chain of lysine (K); O: backbone carbonyl oxygen (C═O); OG: hydroxyl oxygen atom in the polar side chain of serine (S); [OH]: hydroxyl group in tyrosine (Y).

There were no salt bridges observed across the first interface.

Eight hydrogen bonds were observed distributed across the second interface (A029400072 (D chain) with PD-1 (B chain)):

ISVD A029400072 (D chain)

| Aa | Kabat | Length | Amino acid in |
|---|---|---|---|
| residue | numbering | (Å) | PD-1 (B chain) |
| N55 [ND2] | N54 [ND2] | 3.6 Å | M70 [SD] |
| Y106 [OH] | Y100b [OH] | 2.7 Å | A132 [O] |
| N32 [ND2] | N32 [ND2] | 3.3 Å | I134 [O] |
| N55 [N] | N54 [N] | 3.5 Å | E136 [OE1] |
| S52 [OG] | S52 [OG] | 3.2 Å | E136 [OE2] |
| T58 [O] | T57 [O] | 3.0 Å | K78 [NZ] |
| R45 [O] | R45 [O] | 3.3 Å | K131 [NZ] |
| S104 [O] | S100 [O] | 3.1 Å | Q133 [NE2] |

N: backbone amide nitrogen (—NH); ND2: terminal nitrogen in the side-chain amide group of asparagine (N); NE2: terminal nitrogen in amide of glutamine (Q) or imidazole ring of histidine (H); NZ: terminal amino group nitrogen in the basic side chain of lysine (K); O: backbone carbonyl oxygen (C═O); OE1: one of the two carboxylate oxygen atoms in the side chain of glutamate (E); OE2: the second carboxylate oxygen atom in the side chain of glutamate (E); OG: hydroxyl oxygen atom in the polar side chain of serine (S); [OH]: hydroxyl group in tyrosine (Y); SD: to the sulfur atom in the side chain of methionine (M).

There were no salt bridges observed across the second interface.

CDR1, CDR2 and CDR3 residues form the core of the interface.

### 6.13.4 Comparison of the Binding Modes of A0294047E02, A029400073, A029400072 and PD-L1

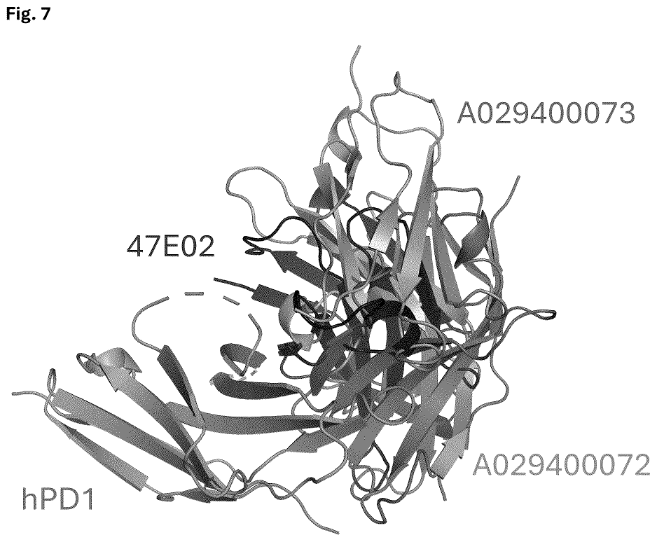

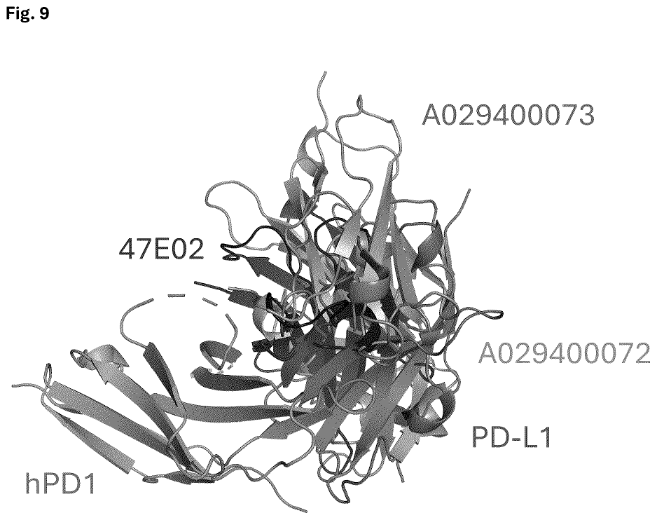

Alignment of the epitope on PD-1 bound by the 3 different ISVD families is shown in FIG. 8A. The ISVD families have a different binding mode but share some common residues. These ISVD families must all compete with each other. This is also reflected in FIG. 7, which shows the 3 ISVDs in complex with human PD-1. FIG. 9 shows the 3 ISVDs and PD-L1 in complex with PD-1 and confirms the competition of the ISVDs with PD-L1 observed in the FACS competition assays.

Alignment of the 3 anti-PD-1 ISVDs is shown in FIG. 8B. The paratopes of the ISVDs are indicated. These ISVD families each have a different paratope.

### Example 14: Design of PD-1/GITR Bispecific Binding Proteins

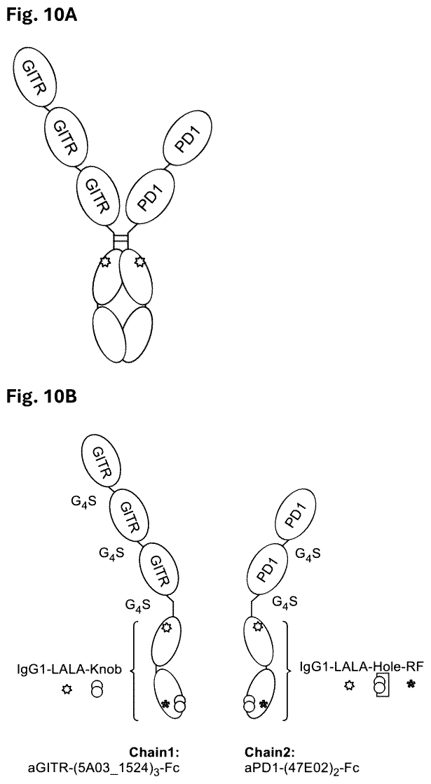

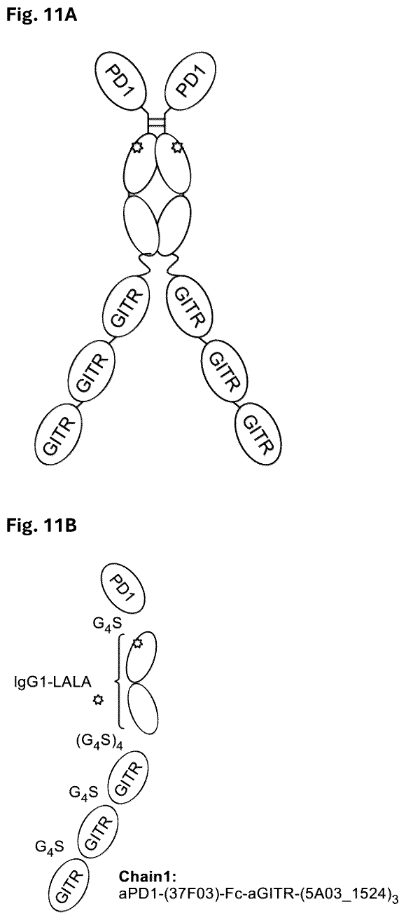

Bispecific binding proteins comprising a first binding moiety that specifically binds to a TNFRSF protein (e.g., a GITR) and a second binding moiety that binds to an immune checkpoint protein (e.g., PD-1) were designed (referred to as “PD-1/GITR bispecific binding protein(s)” or “PD-1/GITR bispecific construct(s)”). The designs of some of the tested constructs are graphically displayed in FIGS. 10 and 11.

Two different pentavalent PD-1/GITR bispecific binding proteins were designed (also referred to herein as “pentavalent anti-PD-1/GITR construct 1 or 2”). The two different pentavalent anti-PD-1/GITR constructs comprised the features shown in FIGS. 10A-10B (and detailed in Table 4 below) which included tandem trivalent anti-human GITR (hGITR) ISVDs as a GITR co-stimulatory receptor agonist and tandem bivalent anti-human PD-1 (hPD-1) ISVDs as a PD-1 co-inhibitory receptor antagonist each fused to the N-terminal Fc portion of a first and a second IgG.

As shown in the FIGS. 11A-11B schematics, an octavalent PD-1/GITR bispecific binding protein was also designed (also referred to herein as, “an octavalent anti-PD-1/GITR construct”; see Table 4 below). The octavalent anti-PD-1/GITR construct included tandem trivalent anti-hGITR ISVDs domains fused to the C-terminal end of a first and second IgG domain (i.e., for a total of six GITR ISVDs in the construct) and an anti-hPD-1 ISVD fused to the N-terminal end of the first and the second IgG domains comprising the Fc portion (i.e., for a total of two PD-1 ISVDs in the construct). Table 4 and Table 5 below show features of the designed constructs and sequences, respectively.

TABLE 4
Construct name and properties of the PD-1/GITR multispecific binding proteins

| | | Valency of | | |
|---|---|---|---|---|
| | | binding | | |
| Name | Construct | moieties | FC domain features | Linker |
| pentavalent | 3x 5A03v1524 + | Three GITR | LALA substitutions (L234A | Gly₄Ser (SEQ ID |
| anti-PD- | 2x 47E02vFW | ISVDs | and L235A in each IgG | NO: 2419) linkage |
| 1/GITR | IgG1 LALA-Fc | Two PD-1 | domain) | between binding |
| construct 1 | (RA17142110) | ISVDs | FC domain features | moieties |
| | SEQ ID NO: | | RF substitutions (H435R in | |
| | 4709 and 4710 | | the first IgG domain and a | |
| | | | Y436F in the second IgG | |
| | | | domain) | |
| | | | Knob and hole technology | |
| | | | Disulfide bridge | |
| pentavalent | 3x 5A03v1524 + | Three GITR | LALA substitutions | Gly₄Ser (SEQ ID |
| anti-PD- | 2x 37F03vFW | ISVDs | RF substitutions | NO: 2419) linkage |
| 1/GITR | IgG1 LALA-Fc | Two PD-1 | Knob and hole technology | between binding |
| construct 2 | (RA17142123) | ISVDs | Disulfide bridge | moieties |
| | SEQ ID NO: | | | |
| | 4712 | | | |
| octavalent | 2x 37F03vFW + | Six GITR | LALA substitutions | Gly₄Ser (SEQ ID |
| anti-PD- | 6x 5A03v1524 | ISVDs | Disulfide bridge | NO: 2419) linkage |
| 1/GITR | IgG1 LALA-Fc | Two PD-1 | | between binding |
| construct | (RA17183505) | ISVDs | | moieties |
| | SEQ ID NO: | | | Extended |
| | 4711 | | | (Gly₄Ser)₄ (SEQ ID |
| | | | | NO: 2426) linker |
| | | | | between a GITR |
| | | | | ISVD and the C- |
| | | | | terminal domain of |
| | | | | IgG |

TABLE 5
Corresponding amino acid sequences for the construct designs shown in Table 4.

| SEQ ID NO/ | |
|---|---|
| Description | Amino Acid sequence |
| SEQ ID NO: 4709 | DVQLVESGGGVVQPGGSLRLSCAASGTFFSIDSMAWYRQAPGKQRELVAAITGGGS |
| RA17142110_HC1 - | PNYADSVKGRFTISSDVAKNTVYLQMNSLRPEDTALYYCNAEGQAGWGTALLDYW |
| Fc + 3xGITR VHH | GQGTLVTVSSGGGGSEVQLVESGGGVVQPGGSLRLSCAASGTFFSIDSMAWYRQAP |
| | GKQRELVAAITGGGSPNYADSVKGRFTISSDVAKNTVYLQMNSLRPEDTALYYCNAE |
| | GQAGWGTALLDYWGQGTLVTVSSGGGGSEVQLVESGGGVVQPGGSLRLSCAASG |
| | TFFSIDSMAWYRQAPGKQRELVAAITGGGSPNYADSVKGRFTISSDVAKNTVYLQM |
| | NSLRPEDTALYYCNAEGQAGWGTALLDYWGQGTLVTVSSGGGGSDKTHTCPPCPA |
| | PEAAGGPSVFLFPPKPKDTLMISRTPEVTCVVVDVSHEDPEVKFNWYVDGVEVHNA |
| | KTKPREEQYNSTYRVVSVLTVLHQDWLNGKEYKCKVSNKALPAPIEKTISKAKGQPRE |
| | PQVYTLPPCRDELTKNQVSLWCLVKGFYPSDIAVEWESNGQPENNYKTTPPVLDSD |
| | GSFFLYSKLTVDKSRWQQGNVFSCSVMHEALHNHYTQKSLSLSPG |
| SEQ ID NO: 4710 | DVQLVESGGGVVQPGGSLRLSCAASGFTLDYYVIGWFRQAPGKEREGVACLSSNGD |
| RA17142110_HC2 - | RINYADSVKGRFTISRDIAKNTVYLQMNSLRPEDTALYHCAAGTSTTVRDMCGIMYLY |
| Fc + 2xPD-1 VHH | DYWGQGTLVTVSSGGGGSEVQLVESGGGVVQPGGSLRLSCAASGFTLDYYVIGWFR |
| | QAPGKEREGVACLSSNGDRINYADSVKGRFTISRDIAKNTVYLQMNSLRPEDTALYH |
| | CAAGTSTTVRDMCGIMYLYDYWGQGTLVTVSSGGGGSDKTHTCPPCPAPEAAGGP |
| | SVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVCTLPP |
| | SRDELTKNQVSLSCAVKGFYPSDIAVEWESNGQPENNYKTTPPVLDSDGSFFLVSKLT |
| | VDKSRWQQGNVFSCSVMHEALHNRFTQKSLSLSPG |
| SEQ ID NO: 4711 | DVQLVESGGGVVQPGGSLRLSCAASGRMHSINAMAWFRQAPGKEREFVALISWG |
| RA17183505 - | NGITYYADSVKGRFTISRDNAKNTLYLQMNSLRPEDTALYYCAAESYGSSWYDSWGQ |
| 3xGITR VHH - Fc - | GTLVTVSSGGGGSDKTHTCPPCPAPEAAGGPSVFLFPPKPKDTLMISRTPEVTCVVVD |
| 1xPD-1 | VSHEDPEVKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGFYPSDIAVE |
| | WESNGQPENNYKTTPPVLDSDGSFFLYSKLTVDKSRWQQGNVFSCSVMHEALHNH |
| | YTQKSLSLSPGGGGGSGGGGSGGGGSGGGGSEVQLVESGGGVVQPGGSLRLSCAA |
| | SGTFFSIDSMAWYRQAPGKQRELVAAITGGGSPNYADSVKGRFTISSDVAKNTVYLQ |
| | MNSLRPEDTALYYCNAEGQAGWGTALLDYWGQGTLVTVSSGGGGSEVQLVESGG |
| | GVVQPGGSLRLSCAASGTFFSIDSMAWYRQAPGKQRELVAAITGGGSPNYADSVKG |
| | RFTISSDVAKNTVYLQMNSLRPEDTALYYCNAEGQAGWGTALLDYWGQGTLVTVSS |
| | GGGGSEVQLVESGGGVVQPGGSLRLSCAASGTFFSIDSMAWYRQAPGKQRELVAAI |
| | TGGGSPNYADSVKGRFTISSDVAKNTVYLQMNSLRPEDTALYYCNAEGQAGWGTAL |
| | LDYWGQGTLVTVSSA |
| SEQ ID NO: 4712 | DVQLVESGGGVVQPGGSLRLSCAASGRMHSINAMAWFRQAPGKEREFVALISWG |
| RA17142123_HC2 - | NGITYYADSVKGRFTISRDNAKNTLYLQMNSLRPEDTALYYCAAESYGSSWYDSWGQ |
| Fc + 2xPD1 VHH | GTLVTVSSGGGGSEVQLVESGGGVVQPGGSLRLSCAASGRMHSINAMAWFRQAP |
| | GKEREFVALISWGNGITYYADSVKGRFTISRDNAKNTLYLQMNSLRPEDTALYYCAAE |
| | SYGSSWYDSWGQGTLVTVSSGGGGSDKTHTCPPCPAPEAAGGPSVFLFPPKPKDTL |
| | MISRTPEVTCVVVDVSHEDPEVKFNWYVDGVEVHNAKTKPREEQYNSTYRVVSVLT |
| | VLHQDWLNGKEYKCKVSNKALPAPIEKTISKAKGQPREPQVCTLPPSRDELTKNQVSL |
| | SCAVKGFYPSDIAVEWESNGQPENNYKTTPPVLDSDGSFFLVSKLTVDKSRWQQGN |
| | VFSCSVMHEALHNRFTQKSLSLSPG |

### Example 15: PD-1/GITR Bispecific Binding Proteins Bind to PD-1 and GITR Positive Cells

### Introduction

In this Example the PD-1/GITR bispecific binding proteins designed in Example 14 were analyzed for the ability to specifically bind to human or cyno PD-1 and to human or cyno GITR expressed on the cell surface of a PD-1 and/or GITR positive cell.

The binding properties of the constructs and controls to bind either GITR (human of cynomolgus GITR) or PD-1 (human of cynomolgus PD-1) expressed on different cell lines or cell types were evaluated by flow cytometry. Binding experiments were performed using the following cell lines or cell types: (a) GloResponse™ NFkB-NlucP/HEK293 cell Lines (Promega, Cat. No. CS188801) stably transfected with human or cynomolgus monkey GITR; (b) pre-B 300.19 single cell clones stably expressing human or cynomolgus PD-1; and (c) primary human and cynomolgus monkey T Cells (purchased from LPT, Germany) which were activated.

### Methods

Binding experiments using a NFkB-NlucP/HEK293 cell line or pre-B 300.19 single cell clones.

Cells were thawed, washed with assay buffer (DPBS (Gibco, Cat. No. 14190) supplemented with 2% heat inactivated FBS (Sigma, Cat. No. F7254) and 0.05% NaN₃ (Acros, Cat. No. 19038)) and seeded in 384-well Bio-One V-bottom plates (Greiner, Cat. No. 781280), with a total of 3×10⁴ cells per well. Serial dilutions of the bispecific molecules and controls (starting from 250 nM final concentration, 3-fold dilution, 11 points, diluted in assay buffer), were added and incubated for 30 minutes at 4° C. Plates were then washed 3 times and cells were resuspended in AffiniPure F(Ab′)2 Fragment Goat Anti-Human IgG, Fcg Fragment Specific (Jackson ImmunoResearch, Cat. No. 109-136-170) (250-fold diluted in assay buffer) for 30 minutes at 4° C. Plates were washed 3 times and cells were resuspended in DAPI Solution (BD Pharmingen, Cat. No. 564907, 5000-fold diluted in assay buffer). Cell suspensions were analyzed with iQue Screener 3 (Sartorius, Intellicyt). In each washing step, plates were centrifuged for 2 minutes at 300×g at 4° C., supernatants discarded, and buffer dispensed by Biotek ELX405 microplate washer (BioTek). Other reagents were added either manually or with ViaFlo (Integra). EC50 s were estimated by dose response modelling. Curves were fitted using 4 parameter logistic regression in GraphPad (GraphPad Software Inc.).

### Binding to Primary Cynomolgus T Cells

Primary Cynomolgus monkey T cells were purchased from LPT, Germany, and were pre-activated with Dynabeads Goat anti-Mouse IgG (Gibco, #11033) coated with mouse anti-Human CD3 clone SP34-2 (BD Bioscience, #551916) and mouse anti-Human CD28 clone CD28-2 (Antibodies Online, #ABIN6384335) at a 1:1 bead: cell ratio in RPMI-1640 (Sigma-Aldrich, #72400) supplemented with 10% FBS (Sigma-Aldrich, #F7524) and 1% Penicillin-Streptomycin (P/S, Sigma-Aldrich, #15140-122). Four, 7 and 11 days after start of incubation at 37° C., the beads were removed and replaced by new beads, again at a 1:1 bead: cell ratio. After a total incubation of 14 days, the beads were removed, and cells were frozen until use. Expression of GITR was confirmed via flow cytometry.

### Results

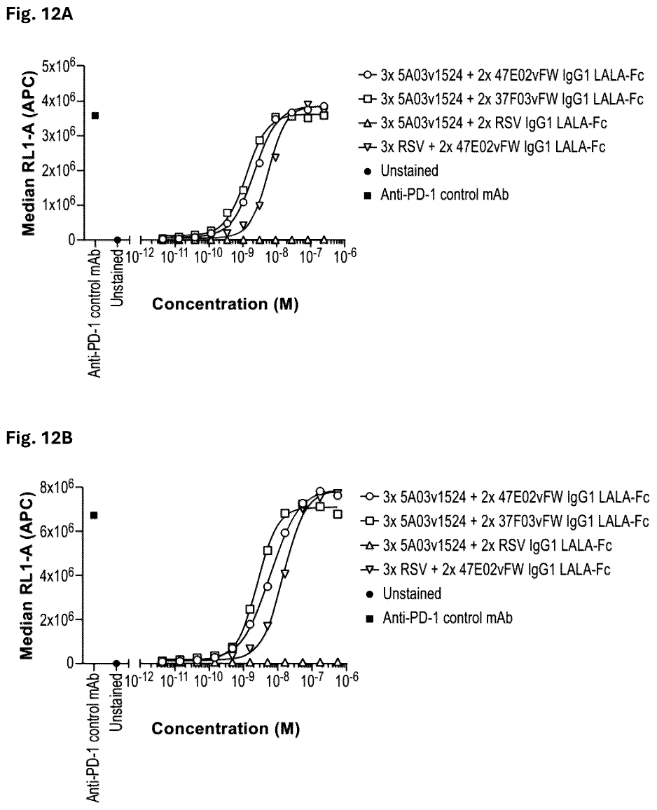

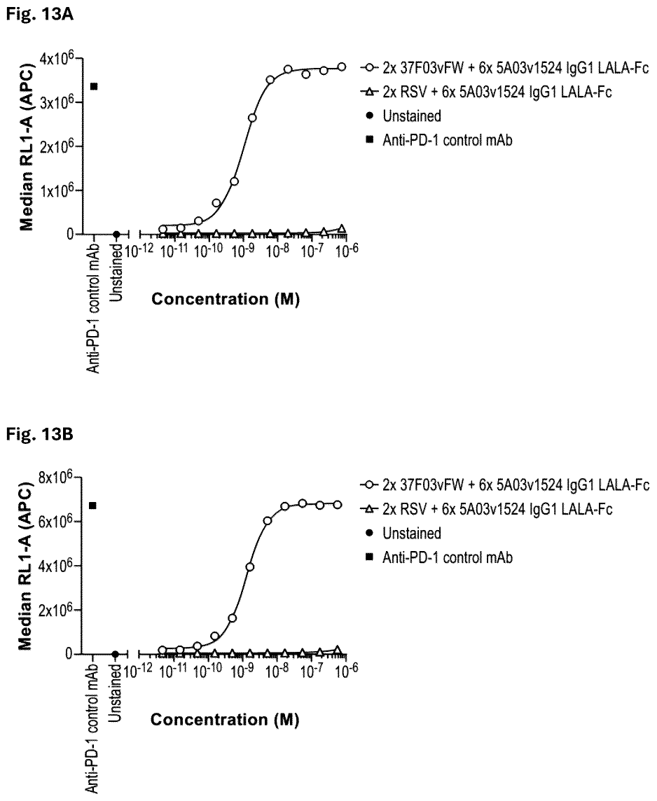

As shown in FIGS. 12 and 13, all constructs exhibiting an anti-PD-1 binding moiety showed specific binding for both human and cyno PD-1 expressed on the cell surface of pre-B 300.19 cells whereas control molecules containing a GITR moiety associated with VHH control which binds to a Respiratory Syncytial Virus (RSV) sequence (i.e., an irrelevant sequence) did not bind the cell surface of pre-B 300.19 cells.

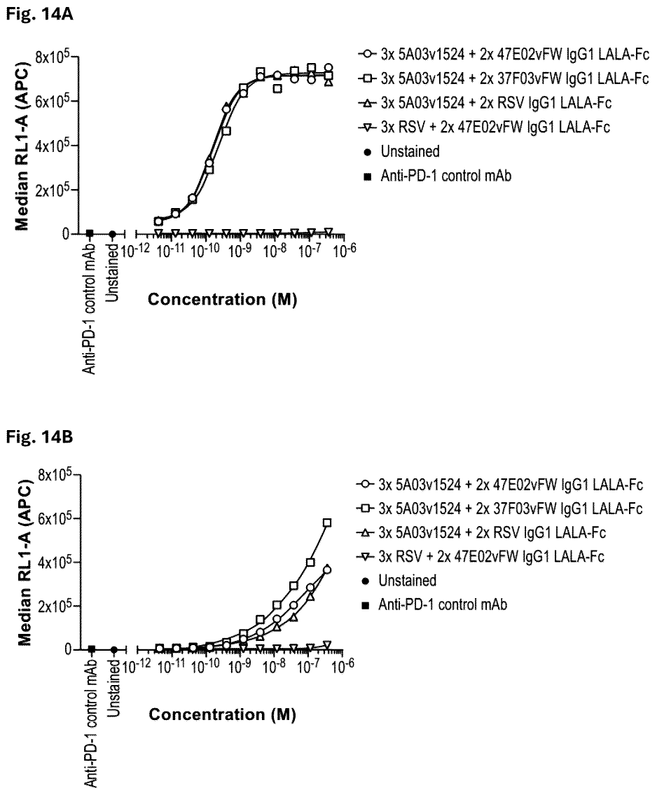

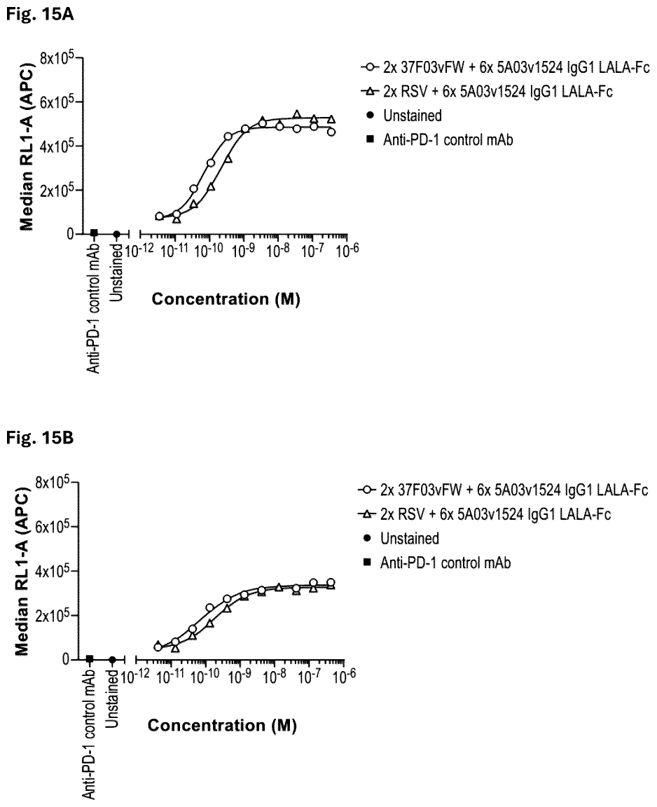

As shown in FIGS. 14A and 15A, all bispecific molecules containing GITR ISVDs actively bound human GITR expressing-NFkB-NlucP/HEK293 cell lines. On the contrary, control molecules that do not exhibit GITR ISVDs did not bind demonstrating the specificity for human GITR of the pentavalent and octavalent bispecific anti-PD-1/GITR constructs. Additionally, the octavalent anti-PD-1/GITR construct bound cynoGITR-expressing NFkB-NlucP/HEK293 cells in a dose-dependent manner (FIG. 15B). Binding of pentavalent anti-PD-1/GITR constructs were also observed to cynoGITR-expressing NFkB-NlucP/HEK293 cells but no saturation of the binding signal was observed preventing calculation of an EC50 (FIG. 14B).

As shown in FIG. 16A, all bispecific and control molecules bound activated primary human T cells. When compared to the monospecific control (3×RSV+2×47E02vFW IgG1 LALA-Fc) the pentavalent anti-PD-1/GITR constructs displayed an increased binding capacity highlighting the added value of bispecificity for binding to primary human T cells. Bispecific molecules also had the capacity to bind activated primary cyno T cells (FIG. 16B). As shown in FIG. 16B, this capacity is preferentially mediated by the anti-PD-1 ISVD. As observed on engineered cell lines, the binding capacity of GITR ISVD on cyno GITR is limited but slightly enhanced when formatted as hexavalent ISVD.

Table 6 summarizes the EC50 (M) values for cell binding with constructs tested in FIGS. 12-16.

TABLE 6
PD-1/GITR bispecific binding proteins binding profiles

| | Binding EC50 (M) | | | | | |
|---|---|---|---|---|---|---|
| | | | | | activated | activated |
| | hPD- | cyPD- | hGITR- | cyGITR- | human | cyno |
| Molecules | 1-preB | 1-preB | HEK293 | HEK293 | T cells | T cells |
| 3x 5A03v1524 + | 2.28 × 10⁻⁹ | 3.63 × 10⁻⁹ | 1.48 × 10⁻¹⁰ | nd | 2.33 × 10⁻¹⁰ | 5.26 × 10⁻⁹ |
| 2x 47E02vFW | | | | | | |
| IgG1 LALA-Fc | | | | | | |
| 3x 5A03v1524 + | 1.30 × 10⁻⁹ | 1.64 × 10⁻⁹ | 2.09 × 10⁻¹⁰ | nd | 9.84 × 10⁻¹¹ | 3.33 × 10⁻⁹ |
| 2x 37F03vFW | | | | | | |
| IgG1 LALA-Fc | | | | | | |
| 3x 5A03v1524 + | nd | nd | 1.38 × 10⁻¹⁰ | nd | 2.50 × 10⁻¹⁰ | nd |
| 2x RSV IgG1 | | | | | | |
| LALA-Fc | | | | | | |
| 3x RSV + 2x | 6.58 × 10⁻⁹ | 8.39 × 10⁻⁹ | nd | nd | 6.01 × 10⁻⁹ | 1.01 × 10⁻⁸ |
| 47E02vFW | | | | | | |
| IgG1 LALA-Fc | | | | | | |
| 2x 37F03vFW + | 5.95 × 10⁻¹⁰ | 8.44 × 10⁻¹⁰ | 7.26 × 10⁻¹¹ | 5.78 × 10⁻¹¹ | 8.57 × 10⁻¹² | 1.89 × 10⁻⁹ |
| 6x 5A03v1524 | | | | | | |
| IgG1 LALA-Fc | | | | | | |
| 2x RSV + 6x | nd | nd | 2.23 × 10⁻¹⁰ | 1.63 × 10⁻¹⁰ | 2.37 × 10⁻¹⁰ | 1.58 × 10⁻⁹ |
| 5A03v1524 | | | | | | |
| IgG1 LALA-Fc | | | | | | |

nd: not determined

### Conclusion

The PD-1/GITR bispecific binding proteins designed herein can bind PD-1 and GITR on PD-1 and GITR positive cells.

### Example 15: PD-1/GITR Bispecific Binding Protein Stimulates GITR Activation and PD-1 Blockade

### Introduction

Example 15 tests whether the PD-1/GITR bispecific binding proteins designed in Example 1 and bound to PD-1 and GITR positive cells can stimulate GITR activation and PD-1 blockade relative to control molecules.

### Methods

### Functional Testing of Bispecific ISVD-Fc Fusion Molecules in Nano-Glo® Luciferase Assay.

The bispecific and control molecules were tested for GITR activation in Nano-Glo® Luciferase assays (Promega, Cat. No. J1252). GloResponse™ NFkB-NlucP/HEK293 cell Lines (Promega, Cat. No. CS188801), stably transfected with human or cynomolgus monkey GITR, were thawed in cell medium DMEM (Gibco, Cat. No. 31966) supplemented with 10% heat inactivated FBS (Sigma, F7254) and seeded in 96-well cell culture treated plates (Corning, Cat. No. 3917) at 15,000 cells per well. Serial dilutions of the tested molecules (starting from 100 nM final concentration) or recombinant human GITR Ligand/TNFSF18 (R&D Systems, C. No 6987-GL) at 100 nM were added and incubated for 5 hours in a 5% CO2 atmosphere at 37° C. After incubation, Nano-Glo™ Luciferase substrate (Promega, Cat. No. N1130) was added and luminescent signal was acquired using CLARIOstar (BMG Labtech).

### Functional Testing of Bispecific and Control Molecules in GloResponse™ NFAT-RE/PD-1 Jurkat Reporter Assay

The bispecific and control molecules were tested for PD-1 blockade in a GloResponse™ NFAT-RE/PD-1 Jurkat reporter assay (Promega, Cat. No. J1252). Two days prior to the assay, GloResponse™ NFAT-RE/PD-1 Jurkat cells were thawed and cultured in Jurkat culture medium (RPMI 1640 Medium, GlutaMAX™ Supplement, HEPES (Gibco, Cat. No. 72400)) supplemented with 10% heat inactivated FBS (Sigma, F7254) for 48 hours in a 5% CO2 atmosphere at 37° C. On the day prior to the assay, PD-L1 aAPC CHO K1 cells were thawed in culture medium (Ham's F-12 Nutrient Mix, GlutaMAXIM supplement (Gibco, Cat No. 31765-068) supplemented with 10% heat inactivated FBS (Sigma, F7254) and seeded in 384-well tissue culture treated plates (Nunc, Cat. No. 3570) at 10,000 cells per well and incubated overnight in a 5% CO2 atmosphere at 37° C. After disposal of the medium of the culture plates, Gloresponse™ NFAT-RE/PD-1 Jurkat cells were added at 15,000 cells/well in Jurkat culture medium containing 1% heat inactivated FBS. Serial dilutions of the tested molecules (starting from 330 nM final concentration) or anti-PD-1 control mAb (Nivolumab) at 250 nM were added and incubated for 24 hours in a 5% CO2 atmosphere at 37° C. After incubation, Bio-Glo™ Luciferase substrate (Promega, Cat. No. G7940) was added and luminescent signal was acquired using CLARIOstar (BMG Labtech).

### Results

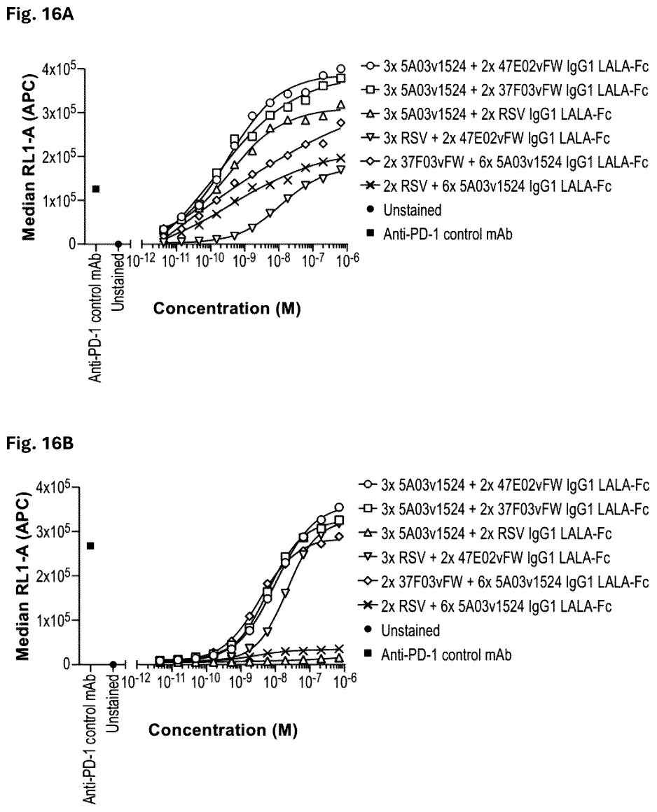

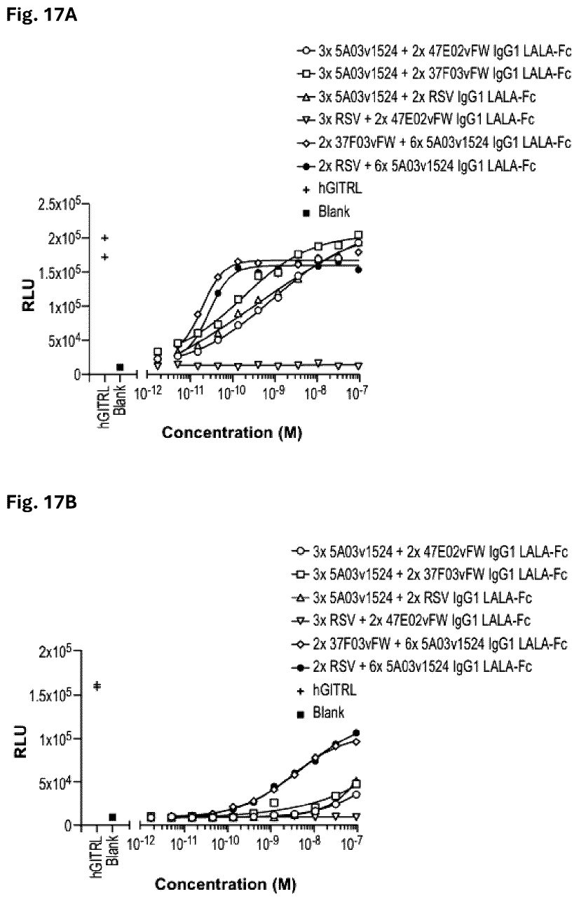

As seen in FIG. 17A, all constructs containing the anti-GITR moiety (5A03v1524), either in a trivalent or a hexavalent form, induced human GITR signaling that is comparable to that of the recombinant hGITRL control. In addition, constructs containing a hexavalent form of anti-GITR (e.g., the octavalent anti-PD-1/GITR construct) display an increased potency as compared to constructs containing a trivalent form of 5A03v1524 (e.g., the pentavalent anti-PD-1/GITR construct). This increased capacity to trigger GITR signaling is also reflected in the cyno GITR reporter assay, in which hexavalent GITR formats showed activity whereas trivalent GITR formats triggered low signal (FIG. 17B).

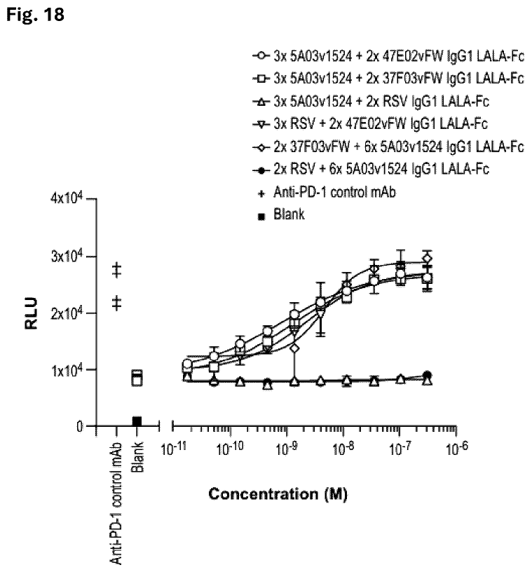

As illustrated in FIG. 18 by the restoration of signaling in reporter cells, all constructs containing an active human anti-PD-1 moiety (47E01vFW or 37F03FW) efficiently blocked the PD-1/PD-L1 interaction and to a similar extent than that of anti-PD-1 control monoclonal antibody (mAb).

Table 7 below summarizes the EC50 [M] and IC50 [M] values obtained in the GITR activation (FIGS. 17A and 17B) and PD-1 blockade (FIG. 18) reporter assays.

TABLE 7
PD-1/GITR bispecific binding proteins EC50 [M] and IC50
[M] values for GITR activation and PD-1 blockade

| | hGITR EC50 | cyGITR EC50 | hPD-1 IC50 |
|---|---|---|---|
| pentavalent anti-PD-1/GITR construct 1 | 8,263 × 10⁻¹⁰ | nd | 8,304 × 10⁻¹⁰ |
| (3x 5A03v1524 + 2x 47E02vFW IgG1 LALA-Fc) | | | |
| pentavalent anti-PD-1/GITR construct 2 | 1,655 × 10⁻¹⁰ | nd | 1,247 × 10⁻⁹ |
| (3x 5A03v1524 + 2x 37F03vFW IgG1 LALA-Fc) | | | |
| octavalent anti-PD-1/GITR construct | 1,79 × 10⁻¹¹ | 3,234 × 10⁻⁹ | 4,775 × 10⁻⁹ |
| 2x 37F03vFW + 6x 5A03v1524 IgG1 LALA-Fc | | | |

nd: not determined

### Conclusion

The PD-1/GITR bispecific binding proteins designed herein upon binding to a PD-1 and GITR positive cell can upregulate GITR signaling and block the PD-1/PD-L1 interaction.

### Example 16: GITR and PD-1 Receptor Occupancy In Vitro Upon Incubation with PD-1/GITR Bispecific Binding Protein

### Introduction

Both GITR clustering required for the induction of T cell activation and PD-1 blockade saturation are both important considerations for maximal efficacy and dosing of a PD-1/GITR bispecific binding protein. Accordingly, this Example tests the GITR and PD-1 receptor occupancy rate upon incubation with the PD-1/GITR bispecific constructs designed and tested in the previous Examples.

### Methods

Experimental methods using NFkB-NlucP/HEK293 cell line and activated T cells were described in Example 14 and 15 above.

Murine T cells were isolated from the spleen of hGITR/hPD-1 KI mice (obtained by crossing C57BL/6-hGITR KI and C57BL/6-hPD KI mice previously generated in collaboration with Center of Imunophenomics-CIPHE (Marseille, France) using EasySep™ Mouse T Cell Enrichment Kit (Stemcell Technologies #19851A). T cells were resuspended in DMEM medium with Glutamax (Gibco #31966-021), 10% FCS (Biowest #S181H-100), 1% penicillin/streptomycin (Gibco #10378-016), 1% NEAA (Gibco #11140-050), 1 mM Na Pyruvate (Gibco #11360-070), 0.05 mM 2-mercaptoethanol (Gibco #31350-010). Resuspended murine T cells were activated in F-bottom 96 well plates previously coated with hamster anti-mouse CD3e antibody (clone 145-2C11; BD Biosciences #567115) at 1 μg/ml, in the presence of syrian hamster anti-mouse CD28 antibody (clone 37.51; eBioscience 16-0261-82) at 1 μg/ml and 2 UI/ml human IL-2 (Miltenyi Biotec #130-097-746) for 3 days.

After 3 days, activated T cells were transferred in V-bottom plates, washed, resuspended in D-PBS and stained with Fixable Viability Dye eFluor™ 780 (Thermo Fisher #65-0865-14) for 15 min at 4° C. After washing, activated T cells were incubated with serial dilutions of tested compounds for 1 h30 at 4° C. and then washed twice in staining buffer. Activated T cells were then stained for 20 min at 4° C. with the antibodies described in Table 8 below. Activated T cells were then washed and analyzed on a Cytek® Aurora flow cytometer.

TABLE 8
Activated CD4⁺ and CD8⁺ T cells harvested
from hGITR/hPD-1 KI mice staining procedure

| Name | Source | Note |
|---|---|---|
| Anti-mCD3-BV750 | BD Biosciences #746988 | None |
| Anti-mCD8a-BUV395 | BD Biosciences #563786 | None |
| Anti-mCD4-BUV805 | BD Biosciences #741912 | None |
| Anti-hPD-1-BV421 | Biolegend #329920 | Added as a competing |
| | | antibody detecting free |
| | | surface PD-1 |
| Anti-hGITR-BV605 | Biolegend #371214 | Added as a competing |
| | | antibody detecting free |
| | | surface GITR |
| Anti-hPD-1 | — | Added as a non- |
| ISVD-A647 | | competing agent |
| | | detecting total PD-1 |
| Anti-hGITR | — | Added as a non- |
| ISVD-A488 | | competing agent |
| | | detecting total GITR |

To determine receptor occupancy (% RO), median fluorescence intensities of binding (MFI) were normalized as follow:
a = Normalized Total surface receptor MFI = “total MFI” at tested concentration [ct] × 100/“total MFI” at baseline [c0]
b = Normalized Free surface receptor MFI = “free MFI”[ct] × 100/“free MFI”[c0]
where “total MFI” refers to MFI obtained with agents detecting total PD-1 or GITR and “free MFI” refers to MFI obtained with agents detecting free PD-1 or GITR.
% RO was then calculated as follow:
% RO = ((a − b)/a) × 100 for each concentration tested [ct]

### Results

As shown in FIGS. 19A and 19B, the pentavalent anti-PD-1/GITR construct 1 efficiently engaged GITR and PD-1 expressed at the surface of ex vivo-activated splenic CD4+ and CD8+ T cells from hGITR/hPD-1 KI mice.

### Conclusion

The anti-PD-1/GITR bispecific binding proteins designed herein can saturate cell surface PD-1 and GITR on primary activated T cells.

### Example 17: PD-1/GITR Bispecific Binding Proteins can Activate Primary Human T Cells

### Introduction

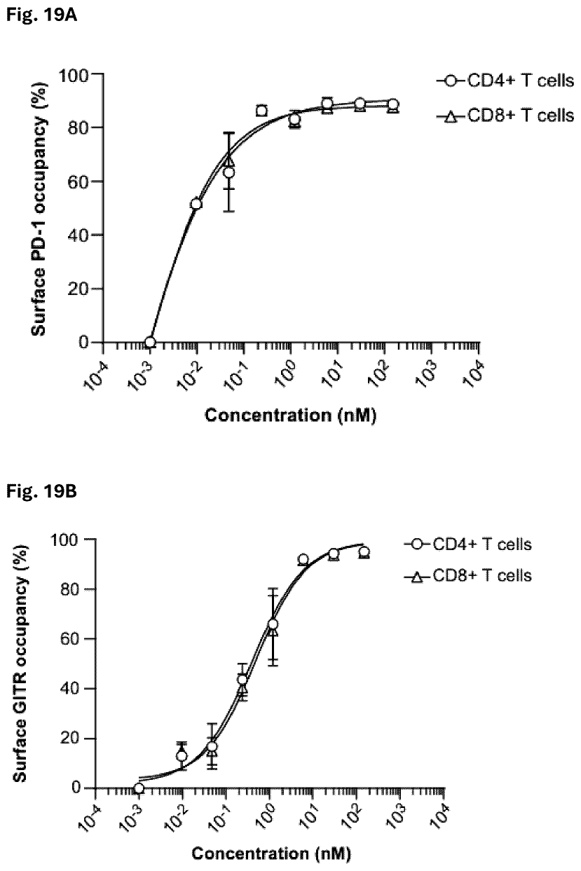

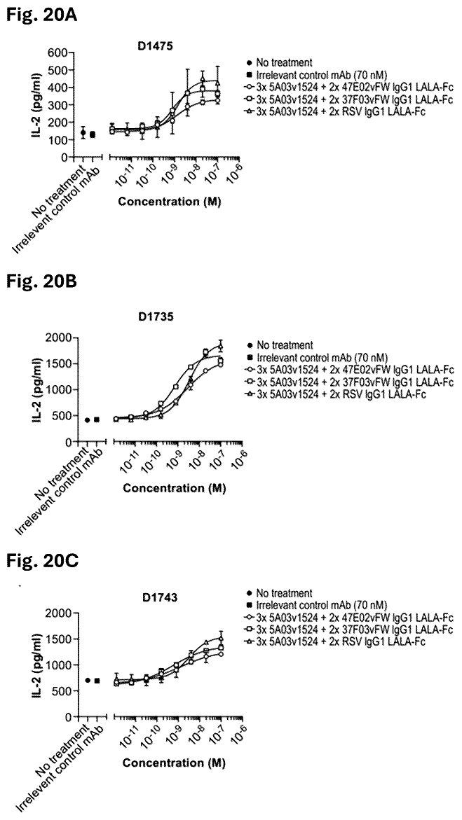

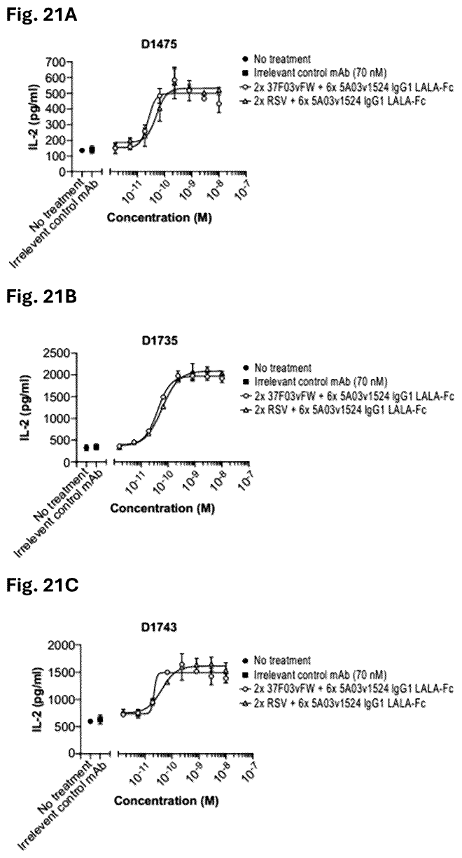

This Example analyzes whether the PD-1/GITR bispecific binding proteins designed and tested in Examples 14-16 can activate primary human T cells obtained from three independent human donors (donors: D1475, D1735, and D1743 are shown in FIGS. 20 and 21).

### Methods

Primary human CD3+ T cells were isolated from frozen human PBMCs using an EasySep™ Human T Cell Isolation Kit (Stemcell Technologies, #17951). Obtained T cells were pre-activated with Human T-Activator CD3/CD28 Dynabeads® (Gibco, #11132D) at a 1:2 bead: cell ratio in RPMI-1640 (Gibco, #72400-021) supplemented with 10% FBS (Sigma-Aldrich, #F7524) and 1% Penicillin-Streptomycin (P/S, Sigma-Aldrich, #15140-122). After 3 days incubation, the beads were removed and replaced by new beads, again at a 1:2 bead: cell ratio. After 4 days incubation, the beads were removed, and cells were frozen until use.

Falcon flat bottom 96-well plates (BD Biosciences, #353072) were coated 1 day prior to the assay with 2 μg/mL anti-CD3 (eBioscience, 16-0037-85) diluted in DPBS (Gibco, 14190-136). After overnight incubation at 4° C., plates were washed with DPBS. Test compounds were diluted in RPMI-1640 supplemented with 10% human AB serum (BiolVT, #SM-612-Hsi) and 1% P/S and added to the plates. Pre-activated T cells were thawed, resuspended in the above-mentioned assay medium, and 200,000 cells/well were added to the plate. Plates were incubated for 24 hours at 37° C., after which cell supernatant was collected for measurement of human IL-2 via ELISA (BD-Pharmingen, capture Ab #555051, detection Ab #555040, IL-2 #554603). Data from three independent donors are shown.

### Results

As shown by the release of IL-2 in FIGS. 20A-C, both pentavalent anti-PD-1/GITR constructs 1 and 2 as well as the GITR monospecific control molecules induced T cell activation in a soluble manner in contrast to the irrelevant isotype control demonstrating the soluble agonist activity of the GITR ISVDs used in the constructs.

The octavalent anti-PD-1/GITR constructs also promoted human T cell activation in a soluble manner as shown by the IL-2 release from T cells in FIGS. 21A-C. Similar to the pentavalent anti-PD-1/GITR constructs, the soluble agonist activity of the octavalent anti-PD-1/GITR constructs was dependent on the GITR-targeting moiety as GITR monospecific control formats displayed the same activity. The impact of GITR-containing molecules was confirmed for each construct on at least 3 independent donors. The mean EC50 of IL-2 secretion induced by the bispecific constructs or controls in a T cell activation assay, calculated from the 3 independent donors are shown below in Table 9.

TABLE 9
Mean EC50 [M] of IL-2 secretion from T cells
induced by PD-1/GITR bispecific binding proteins

| | Mean EC50 [IL-2] ± |
|---|---|
| Construct Name | SD (pg/ml) |
| pentavalent anti-PD-1/GITR construct 1 | 1.73 ± 0.92 × 10⁻⁹ |
| (3x 5A03v1524 + 2x 47E02vFW IgG1 LALA-Fc) | |
| pentavalent anti-PD-1/GITR construct 2 | 6.82 ± 1.12 × 10⁻¹⁰ |
| (3x 5A03v1524 + 2x 37F03vFW IgG1 LALA-Fc) | |
| pentavalent control | 2.66 ± 1.06 × 10⁻⁹ |
| (3x 5A03v1524 + 2x RSV IgG1 LALA-Fc) | |
| octavalent anti-PD-1/GITR construct | 2.90 ± 1.04 × 10⁻¹¹ |
| 2x 37F03vFW + 6x 5A03v1524 IgG1 LALA-Fc | |
| octavalent control | 5.08 ± 0.92 × 10⁻¹¹ |
| 2x RSV + 6x 5A03v1524 IgG1 LALA-Fc | |

### Conclusion

The PD-1/GITR bispecific binding proteins designed herein can activate primary human T cells as measured by IL-2 secretion.

### Example 18: PD-1/GITR Bispecific Binding Proteins Enhanced Induction of T Cell Activation in a MLR Assay

### Introduction

This example analyzes the ability of the PD-1/GITR bispecific binding proteins described herein to modulate the interaction between an antigen presenting cell and T cells by using a mixed lymphocyte reaction (MLR) assay.

### Methods

Immature dendritic cells (iDC) were generated from healthy donor monocytes. Monocytes were isolated from frozen human PBMC by negative selection (Stemcell Technologies #19359) and cultured for 7 days in RPMI 1640 (Sigma-Aldrich, #72400), 10% FCS (Sigma-Aldrich, #F7524), 1% penicillin/streptomycin (Sigma-Aldrich, #15140-122) supplemented with 20 ng/ml human IL-4 (Biolegend, #B341746) and 100 ng/ml human GM-CSF (Biolegend, #572904) with replenishment of IL-4 and GM-CSF on day 3. iDC were harvested on day 7 and frozen until use. On the day of MLR, iDC from a first donor were thawed and plated in 96-well F-bottom plates (Costar, #3596) in CTS AIM V SFM medium (Gibco, #0870112-DK) supplemented with 5% FBS and 1% P/S.

CD4⁺ T cells were isolated from frozen human PBMC by negative selection and resuspended in supplemented CTS AIM V SFM medium.

iDC and autologous CD4⁺ T cells were co-cultured at a 1:10 ratio in supplemented CTS AIM V SFM medium in the presence of serially diluted test compounds or anti-PD-1 control mAb (Nivolumab) at 200 nM. Plates were incubated for 4 days at 37° C., after which cell supernatant was collected for measurement of human GM-CSF via ELISA (R&D Systems, #DY215).

### Results

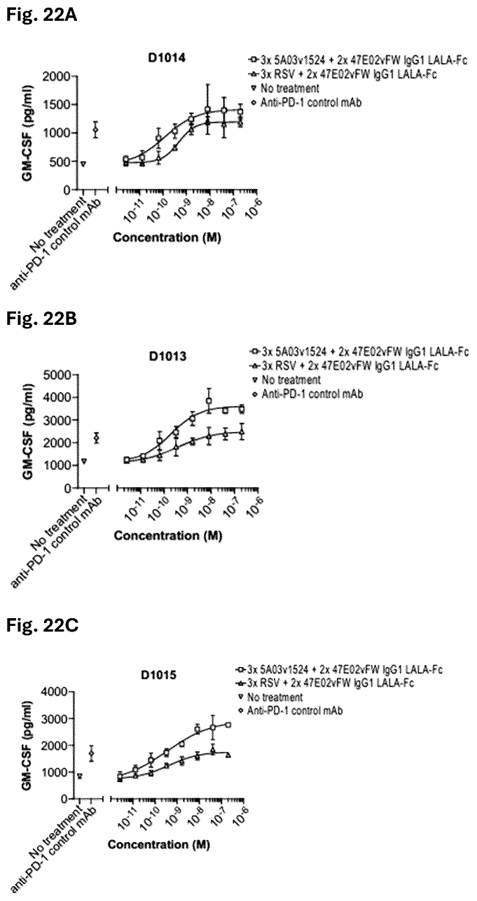

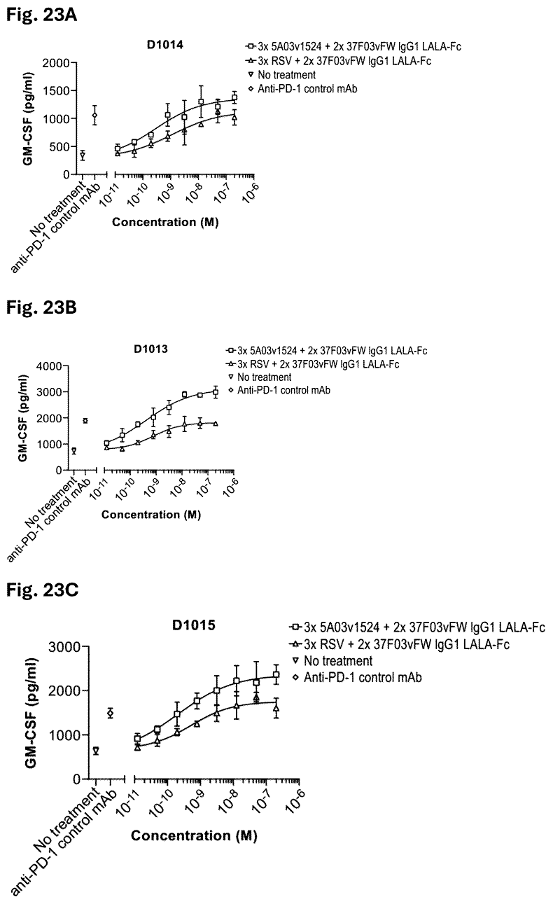

As shown in FIGS. 22A-22C and 23A-23C, both pentavalent anti-PD-1/GITR constructs 1 and 2 induced T cell activation in a MLR assay as illustrated by GM-CSF release. The activity of the anti-PD-1 arm of the bispecific binding proteins was shown by the dose-dependent effect of the PD-1 monospecific control. Additional effect of the GITR ISVD is shown by the higher GM-CSF release induced by the pentavalent anti-PD-1/GITR constructs as compared to their respective monospecific PD-1 controls and anti-PD-1 control mAb.

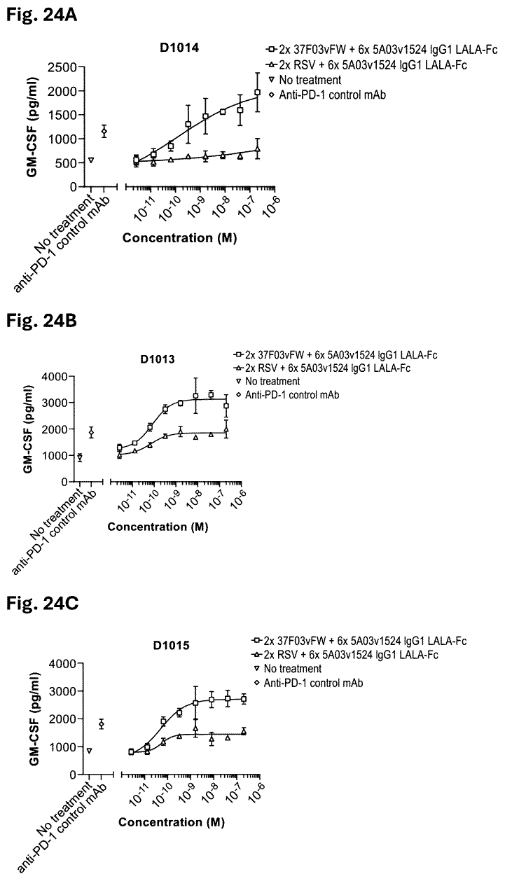

Additionally, the octavalent anti-PD-1/GITR construct also induced T cell activation in a MLR assay as illustrated by GM-CSF secretion in a dose-dependent manner (FIG. 24A-24C). The impact of both GITR and PD-1 binding moieties is demonstrated by the higher activity, as measured by GM-CSF secretion, of the bispecific anti-PD-1/GITR constructs as compared to the “GITR monospecific” control molecule and anti-PD-1 control mAb.

### Conclusion

This PD-1/GITR bispecific binding proteins described herein can modulate the interaction between an antigen presenting cell and T cells in a MLR assay resulting in enhanced induction of T cell activation as measured GM-CSF secretion. Additionally, the MLR assay allows for visualization of both GITR agonism and PD-1 blockade.

### Example 19: PD-1/GITR Bispecific Binding Proteins Attenuate Tumor Volume In Vivo

### Introduction

This Example tests the in vivo efficacy of the PD-1/GITR bispecific binding proteins described herein ability to reduce tumor volume in hGITR/hPD-1 KI mice.

### Method

### Generation of Mice

Knock-in mouse model expressing humanized version of Tnfrsf18 (GITR) and humanized version of Pdcd1 (PD-1) were obtained by crossing C57BL/6-hGITR KI and C57BL/6-hPD KI mice previously generated in collaboration with The Center for ImmunoPHEnomics-CIPHE (Marseille, France). All animals were bred and housed at the Sanofi specific-pathogen-free facilities in ventilated cages, with a standard 12/12-h dark/light cycle at 22° C. ambient temperature and controlled humidity. For all in vivo experiments, animals of both sexes were used between the ages of 10 and 17 weeks, randomized and assigned to experimental groups. All in vivo studies were done in accordance with institutional committees in an accredited facility with all compliance to French and European guidelines and requirements for animal care.

### Tumor Challenge and Treatment

MC38 murine colon adenocarcinoma cells (1×10⁶) resuspended in 200 μL of D-PBS were implanted subcutaneously in the right flank of mice, and tumor volumes were measured every 2-3 days by electronic caliper. Tumor volume was calculated using the formula (L2×L1)/2, where L1 and L2 represent the longest and shortest diameter, respectively. When tumor volume reached the range of 50 mm³ to 145 mm³ (9 days after tumor inoculation), mice were randomized by tumor size and sex and treated by intraperitoneal injection twice a week for two weeks (day 9; day 12; day 16; day 19). Mice were monitored for 25 days, or until the majority of the control group was euthanized due to ethical limitations on tumor size.

### Results

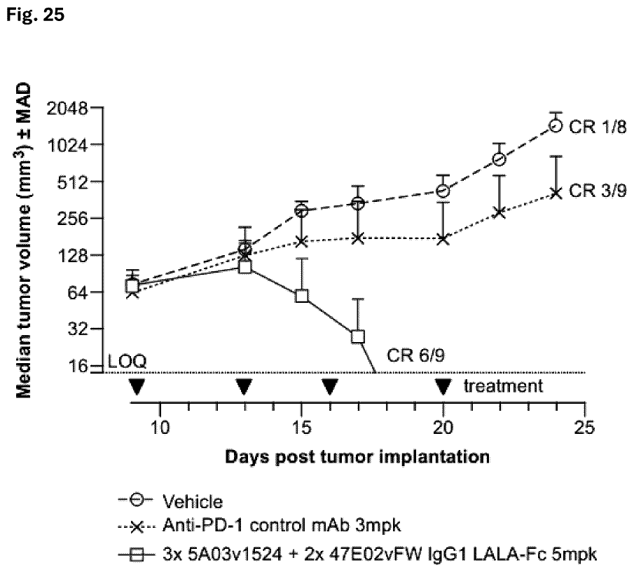

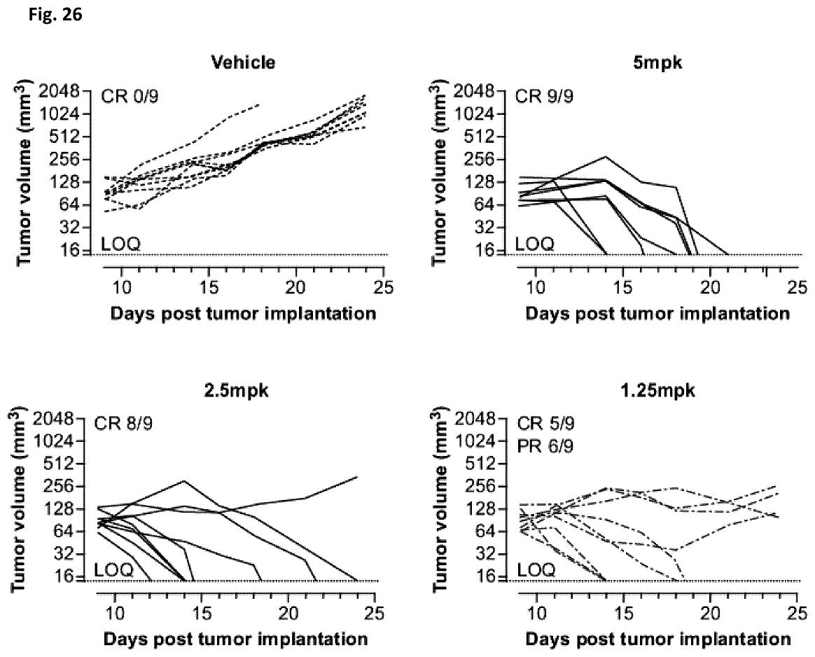

The pentavalent anti-PD-1/GITR construct 1 showed strong anti-tumor activity, superior to anti-PD-1 control mAb at equimolar dose, in MC38-bearing hGITR/hPD-1 mice as shown in FIG. 25. In addition, anti-tumor activity of pentavalent anti-PD-1/GITR construct 1 was dose-dependent as observed in FIG. 26.

### Conclusion

The PD-1/GITR bispecific binding proteins described herein have the ability to reduce tumor volume in a hGITR/hPD-1 KI mice.

GITR/PD-1 Multispecific Sequence Table

| SEQ ID NO/ | |
|---|---|
| Description | Amino Acid sequence |
| SEQ ID NO: 4709 | DVQLVESGGGVVQPGGSLRLSCAASGTFFSIDSMAWYRQAPGKQRELVAAITGG |
| RA17142110_HC1 - | GSPNYADSVKGRFTISSDVAKNTVYLQMNSLRPEDTALYYCNAEGQAGWGTALL |
| Fc + 3xGITR VHH | DYWGQGTLVTVSSGGGGSEVQLVESGGGVVQPGGSLRLSCAASGTFFSIDSMA |
| | WYRQAPGKQRELVAAITGGGSPNYADSVKGRFTISSDVAKNTVYLQMNSLRPED |
| | TALYYCNAEGQAGWGTALLDYWGQGTLVTVSSGGGGSEVQLVESGGGVVQPG |
| | GSLRLSCAASGTFFSIDSMAWYRQAPGKQRELVAAITGGGSPNYADSVKGRFTIS |
| | SDVAKNTVYLQMNSLRPEDTALYYCNAEGQAGWGTALLDYWGQGTLVTVSSG |
| | GGGSDKTHTCPPCPAPEAAGGPSVFLFPPKPKDTLMISRTPEVTCVVVDVSHEDP |
| | EVKFNWYVDGVEVHNAKTKPREEQYNSTYRVVSVLTVLHQDWLNGKEYKCKVS |
| | NKALPAPIEKTISKAKGQPREPQVYTLPPCRDELTKNQVSLWCLVKGFYPSDIAVE |
| | WESNGQPENNYKTTPPVLDSDGSFFLYSKLTVDKSRWQQGNVFSCSVMHEALH |
| | NHYTQKSLSLSPG |
| SEQ ID NO: 4710 | DVQLVESGGGVVQPGGSLRLSCAASGFTLDYYVIGWFRQAPGKEREGVACLSSN |
| RA17142110_HC2 - | GDRINYADSVKGRFTISRDIAKNTVYLQMNSLRPEDTALYHCAAGTSTTVRDMCG |
| Fc + 2xPD1 VHH | IMYLYDYWGQGTLVTVSSGGGGSEVQLVESGGGVVQPGGSLRLSCAASGFTLDY |
| | YVIGWFRQAPGKEREGVACLSSNGDRINYADSVKGRFTISRDIAKNTVYLQMNSL |
| | RPEDTALYHCAAGTSTTVRDMCGIMYLYDYWGQGTLVTVSSGGGGSDKTHTCP |
| | PCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISK |
| | AKGQPREPQVCTLPPSRDELTKNQVSLSCAVKGFYPSDIAVEWESNGQPENNYKT |
| | TPPVLDSDGSFFLVSKLTVDKSRWQQGNVFSCSVMHEALHNRFTQKSLSLSPG |
| SEQ ID NO: 4711 | DVQLVESGGGVVQPGGSLRLSCAASGRMHSINAMAWFRQAPGKEREFVALISW |
| RA17183505 - 3xGITR | GNGITYYADSVKGRFTISRDNAKNTLYLQMNSLRPEDTALYYCAAESYGSSWYDS |
| VHH - Fc - 1xPD1 | WGQGTLVTVSSGGGGSDKTHTCPPCPAPEAAGGPSVFLFPPKPKDTLMISRTPEV |
| | TCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQQGNV |
| | FSCSVMHEALHNHYTQKSLSLSPGGGGGSGGGGSGGGGSGGGGSEVQLVESGG |
| | GVVQPGGSLRLSCAASGTFFSIDSMAWYRQAPGKQRELVAAITGGGSPNYADSV |
| | KGRFTISSDVAKNTVYLQMNSLRPEDTALYYCNAEGQAGWGTALLDYWGQGTL |
| | VTVSSGGGGSEVQLVESGGGVVQPGGSLRLSCAASGTFFSIDSMAWYRQAPGK |
| | QRELVAAITGGGSPNYADSVKGRFTISSDVAKNTVYLQMNSLRPEDTALYYCNAE |
| | GQAGWGTALLDYWGQGTLVTVSSGGGGSEVQLVESGGGVVQPGGSLRLSCAA |
| | SGTFFSIDSMAWYRQAPGKQRELVAAITGGGSPNYADSVKGRFTISSDVAKNTVY |
| | LQMNSLRPEDTALYYCNAEGQAGWGTALLDYWGQGTLVTVSSA |
| SEQ ID NO: 4712 | DVQLVESGGGVVQPGGSLRLSCAASGRMHSINAMAWFRQAPGKEREFVALISW |
| RA17142123_HC2 - | GNGITYYADSVKGRFTISRDNAKNTLYLQMNSLRPEDTALYYCAAESYGSSWYDS |
| Fc + 2xPD1 VHH | WGQGTLVTVSSGGGGSEVQLVESGGGVVQPGGSLRLSCAASGRMHSINAMAW |
| | FRQAPGKEREFVALISWGNGITYYADSVKGRFTISRDNAKNTLYLQMNSLRPEDT |
| | ALYYCAAESYGSSWYDSWGQGTLVTVSSGGGGSDKTHTCPPCPAPEAAGGPSVF |
| | LFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVCTL |
| | PPSRDELTKNQVSLSCAVKGFYPSDIAVEWESNGQPENNYKTTPPVLDSDGSFFL |
| | VSKLTVDKSRWQQGNVFSCSVMHEALHNRFTQKSLSLSPG |
| SEQ ID NO: 4713 | VTVSS |
| C-terminal sequence | |
| SEQ ID NO: 4714 | DVQLVESGGGVVQPGGSLRLSCAASGTFFSIDSMAWYRQAPGKQRELVAAITGG |
| RA*110/*123_VHH | GSPNYADSVKGRFTISSDVAKNTVYLQMNSLRPEDTALYYCNAEGQAGWGTALL |
| anti-GITR- | DYWGQGTLVTVSS |
| (05A03_1524) E1D | |
| SEQ ID NO: 4715 | EVQLVESGGGVVQPGGSLRLSCAASGTFFSIDSMAWYRQAPGKQRELVAAITGG |
| RA*110/*123/*505_V | GSPNYADSVKGRFTISSDVAKNTVYLQMNSLRPEDTALYYCNAEGQAGWGTALL |
| HH anti-GITR- | DYWGQGTLVTVSS |
| (05A03_1524) | |
| SEQ ID NO: 4716 | DVQLVESGGGVVQPGGSLRLSCAASGFTLDYYVIGWFRQAPGKEREGVACLSSN |
| RA*110_VHH anti- | GDRINYADSVKGRFTISRDIAKNTVYLQMNSLRPEDTALYHCAAGTSTTVRDMCG |
| PD1-(47E02)_E1D | IMYLYDYWGQGTLVTVSS |
| SEQ ID NO: 4717 | EVQLVESGGGVVQPGGSLRLSCAASGFTLDYYVIGWFRQAPGKEREGVACLSSN |
| RA*110_VHH anti- | GDRINYADSVKGRFTISRDIAKNTVYLQMNSLRPEDTALYHCAAGTSTTVRDMCG |
| PD1-(47E02) | IMYLYDYWGQGTLVTVSS |
| SEQ ID NO: 4718 | DVQLVESGGGVVQPGGSLRLSCAASGRMHSINAMAWFRQAPGKEREFVALISW |
| RA*123/*505_VHH | GNGITYYADSVKGRFTISRDNAKNTLYLQMNSLRPEDTALYYCAAESYGSSWYDS |
| anti-PD1- | WGQGTLVTVSS |
| (37F03)_E1D | |
| SEQ ID NO: 4719 | EVQLVESGGGVVQPGGSLRLSCAASGRMHSINAMAWFRQAPGKEREFVALISW |
| RA*123/*505_VHH | GNGITYYADSVKGRFTISRDNAKNTLYLQMNSLRPEDTALYYCAAESYGSSWYDS |
| anti-PD1-(37F03) | WGQGTLVTVSS |
| SEQ ID NO: 4720 | GTFFSIDSMA |
| CDR1 GITR | |
| RA*110/*123_VHH | |
| anti-GITR- | |
| (05A03_1524)_E1D | |
| RA*110/*123/*505_VHH | |
| anti-GITR- | |
| (05A03_1524) | |
| SEQ ID NO: 4721 | AITGGGSPN |
| CDR2 GITR | |
| RA*110/*123_VHH | |
| anti-GITR- | |
| (05A03_1524)_E1D | |
| RA*110/*123/*505_VHH | |
| anti-GITR- | |
| (05A03_1524) | |
| SEQ ID NO: 4722 | EGQAGWGTALLDY |
| CDR3 GITR | |
| RA*110/*123_VHH | |
| anti-GITR- | |
| (05A03_1524)_E1D | |
| RA*110/*123/*505_VHH | |
| anti-GITR- | |
| (05A03_1524) | |
| SEQ ID NO: 4723 | GFTLDYYVIG |
| CDR1 PD1 | |
| RA*110_VHH anti- | |
| PD1-(47E02)_E1D & | |
| RA*110_VHH anti- | |
| PD1-(47E02) | |
| SEQ ID NO: 4724 | CLSSNGDRIN |
| CDR2 PD1 | |
| RA*110_VHH anti- | |
| PD1-(47E02)_E1D & | |
| RA*110_VHH anti- | |
| PD1-(47E02) | |
| SEQ ID NO: 4725 | GTSTTVRDMCGIMYLYDY |
| CDR3 PD1 | |
| RA*110_VHH anti- | |
| PD1-(47E02)_E1D & | |
| RA*110_VHH anti- | |
| PD1-(47E02) | |
| SEQ ID NO: 4726 | GRMHSINAMA |
| CDR1 PD1 | |
| RA*123/*505_VHH | |
| anti-PD1- | |
| (37F03)_E1D & | |
| RA*123/*505_VHH | |
| anti-PD1-(37F03) | |
| SEQ ID NO: 4727 | LISWGNGITY |
| CDR2 PD1 | |
| RA*123/*505_VHH | |
| anti-PD1- | |
| (37F03)_E1D & | |
| RA*123/*505_VHH | |
| anti-PD1-(37F03) | |
| SEQ ID NO: 4728 | SYGSSWYDS |
| CDR3 PD1 | |
| RA*123/*505_VHH | |
| anti-PD1- | |
| (37F03)_E1D & | |
| RA*123/*505_VHH | |
| anti-PD1-(37F03) | |

### INDUSTRIAL APPLICABILITY

The ISVDs, polypeptides, constructs, nucleic acid molecules encoding the same, vectors comprising the nucleic acids and compositions described herein may be used for example in the treatment, such as treatment of subjects suffering from cancer.

### 7 Items

### 7.1 Embodiment 1, Family A0294047E02

1. An immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1 protein, wherein at least one amino acid residue in the ISVD is capable of interacting with at least one amino acid residue in at least one, preferably in each, of the following stretches (of the ectodomain) of human PD-1 protein, when numbered in accordance with SEQ ID NO: 4703: V64-P83, and I126-E136, preferably when the ISVD and human PD-1 protein are mixed in in PBS1× buffer and crystallized by hanging drop vapor diffusion using 30% PEG3350, 50 mM TRIS, pH 8 at 19° C.
2. The ISVD according to item 1, wherein the at least one amino acid residue in at least one, preferably in each stretch (of the ectodomain) of human PD-1 protein is selected from:
   For stretch V64-P83: V64, N66, Y68, S73, N74, Q75, T76, D77, K78, and P83;
   For stretch I126-E136: I126, L128, A129, P130, K131, A132, I134, and E136, when numbered in accordance with SEQ ID NO: 4703.
3. The ISVD according to item 1 or 2, wherein at least one amino acid residue located in the ISVD is capable of interacting with at least two, at least three, at least four, at least five, at least six, at least seven, at least eight, at least nine, or all of the following amino acid residues in stretch V64-P83 (of the ectodomain) of human PD-1 protein, when numbered in accordance with SEQ ID NO: 4703: V64, N66, Y68, S73, N74, Q75, T76, D77, K78, and P83.
4. The ISVD according to any one of the preceding items, wherein at least one amino acid residue located in the ISVD is capable of interacting with at least two, at least three, at least four, at least five, at least six, at least seven, or all of the following amino acid residues in stretch I126-E136 (of the ectodomain) of human PD-1 protein when numbered in accordance with SEQ ID NO: 4703: I126, L128, A129, P130, K131, A132, I134, and E136.
5. The ISVD according to any one of the preceding items, wherein at least one amino acid residue located in the ISVD is capable of forming salt bridges with at least the amino acid residue E136 of human PD-1 protein, when numbered in accordance with SEQ ID NO: 4703, preferably when the ISVD and human PD-1 protein are mixed in PBS1× buffer and crystallized by hanging drop vapor diffusion using 30% PEG3350, 50 mM TRIS, pH 8 at 19° C.
6. The ISVD according to any one of the preceding items, wherein at least one amino acid residue located in the ISVD is capable of forming hydrogen bonds with at least one, preferably all, of the following amino acid residues of human PD-1 protein, when numbered in accordance with SEQ ID NO: 4703: N66, Y68, K78, A132, optionally wherein at least one amino acid residue located in the ISVD is further capable of forming hydrogen bonds with the amino acid residue N74 of human PD-1 protein, when numbered in accordance with SEQ ID NO: 4703, preferably when the ISVD and human PD-1 protein are mixed in PBS1× buffer and crystallized by hanging drop vapor diffusion using 30% PEG3350, 50 mM TRIS, pH 8 at 19° C.
7. The ISVD according to any one of the preceding items, wherein the least one amino acid residue located in the ISVD is capable of interacting with an (discontinuous) epitope on human PD-1 comprising at least one amino acid residue in each of the following stretches (of the ectodomain) of human PD-1 protein: V64-P83, and I126-E136, when numbered in accordance with SEQ ID NO: 4703.
8. The ISVD according to item 1, wherein the at least one amino acid residue in each stretch (of the ectodomain) of human PD-1 protein is selected from:
   For stretch V64-P83: V64, N66, Y68, S73, N74, Q75, T76, D77, K78, and P83;
   For stretch I126-E136: I126, L128, A129, P130, K131, A132, I134, and E136, when numbered in accordance with SEQ ID NO: 4703.
9. The ISVD according to any one of the preceding items, wherein at least one amino acid residue located in the ISVD is capable of interacting with at least one, at least two, at least three, at least four, at least five, at least six, at least seven, at least ten, at least fifteen, preferably all, of the following amino acid residues of human PD-1 protein, when numbered in accordance with SEQ ID NO: 4703: V64, N66, Y68, S73, N74, Q75, T76, D77, K78, P83, 1126, L128, A129, P130, K131, A132, I134, and E136, preferably when the ISVD and human PD-1 protein are mixed in PBS1× buffer and crystallized by hanging drop vapor diffusion using 30% PEG3350, 50 mM TRIS, pH 8 at 19° C.
10. The ISVD according to any one of the preceding items, wherein the at least one amino acid residue located in the ISVD that is capable of interacting with amino acid residues of human PD-1 protein is located in CDR3.
11. The ISVD according to any one of the preceding items, wherein the amino acid at position 45 (Kabat numbering) is capable of interacting, preferably by forming a salt bridge, with E136 in PD-1, preferably when the ISVD and human PD-1 protein are mixed in PBS1× buffer and crystallized by hanging drop vapor diffusion using 30% PEG3350, 50 mM TRIS, pH 8 at 19° C.
12. The ISVD according to any one of the preceding items, wherein:
   the amino acid residue at position 100e (Kabat numbering) is capable of interacting, preferably by forming a hydrogen bond, with A132 in PD-1;
   the amino acid residue at position 100h (Kabat numbering) is capable of interacting, preferably by forming a hydrogen bond, with N66 in PD-1;
   the amino acid residue at position 100j (Kabat numbering) is capable of interacting, preferably by forming a hydrogen bond, with N66 in PD-1;
   the amino acid residue at position 100j (Kabat numbering) is capable of interacting, preferably by forming a hydrogen bond, with Y68 in PD-1;
   the amino acid residue at position 100j (Kabat numbering) is capable of interacting, preferably by forming a hydrogen bond, with A132 in PD-1;
   the amino acid residue at position 101 (Kabat numbering) is capable of interacting, preferably by forming a hydrogen bond, with N66 in PD-1; and/or.
   the amino acid residue at position 101 (Kabat numbering) is capable of interacting, preferably by forming a hydrogen bond, with K78 in PD-1;
preferably when the ISVD and human PD-1 protein are mixed in PBS1× buffer and crystallized by hanging drop vapor diffusion using 30% PEG3350, 50 mM TRIS, pH 8 at 19° C.
13. The ISVD according to any one of the preceding items, wherein two amino acids are considered to interact when the distance between them is 4.0 Å or less.
14. The ISVD according to any one of the preceding items, wherein the interacting amino acid residue(s) in the ISVD and in human PD-1 protein are identified by X-ray crystallography by:
   Mixing human PD-1 protein, and the ISVD in PBS1× buffer;
   Concentrating the protein complex to 10.6 mg/ml;
   Crystallizing by hanging drop vapor diffusion using 30% PEG3350, 50 mM TRIS, pH 8 at 19° C.;
   Collecting X-ray diffraction data;
   Processing the collected data, preferably as described in Example 13.
15. The ISVD according to any one of the preceding items, wherein the (discontinuous) epitope comprises or consists of the following amino acid residues of human PD-1 protein, when numbered in accordance with SEQ ID NO: 4703: V64, N66, Y68, T76, D77, K78, P83, 1126, L128, A129, P130, K131, A132, I134, E136.
16. The ISVD according to any one of the preceding items, wherein the (discontinuous) epitope comprises or consists of the following amino acid residues of human PD-1 protein, when numbered in accordance with SEQ ID NO: 4703: V64, N66, Y68, N74, T76, D77, K78, P83, 1126, L128, A129, P130, K131, A132, I134, E136.
17. The ISVD according to any one of the preceding items, wherein the (discontinuous) epitope comprises or consists of the following amino acid residues of human PD-1 protein, when numbered in accordance with SEQ ID NO: 4703: V64, N66, Y68, S73, Q75, T76, D77, K78, P83, 1126, L128, A129, P130, K131, A132, I134, E136.
18. The ISVD according to any one of the preceding items, wherein the (discontinuous) epitope comprises or consists of the following amino acid residues of human PD-1 protein, when numbered in accordance with SEQ ID NO: 4703: V64, N66, Y68, S73, N74, Q75, T76, D77, K78, P83, 1126, L128, A129, P130, K131, A132, I134, E136.
19. The ISVD according to any one of the preceding items, wherein the ISVD cross-blocks the binding to human PD-1 protein of a polypeptide having an amino acid sequence selected from SEQ ID NOs: 1-488, preferably wherein the ISVD cross-blocks the binding to human PD-1 protein of a polypeptide having an amino acid sequence selected from SEQ ID NO: 402, or wherein the binding of the ISVD to human PD-1 protein is cross-blocked by a polypeptide having an amino acid sequence selected from SEQ ID NOs: 1-488.
20. An immunoglobulin single variable domain (ISVD) specifically binding human PD-1 protein, that essentially consists of 4 framework regions (FR1 to FR4, respectively) and 3 complementarity determining regions (CDR1 to CDR3, respectively), wherein the ISVD comprises an amino acid residue capable of forming salt bridges at at least position 45 (Kabat numbering), preferably wherein the ISVD comprises at least the following amino acid residue: R45 (Kabat numbering).
21. The ISVD according to any one of the preceding items, wherein the ISVD comprises an amino acid residue capable of forming hydrogen bonds at at least one, preferably at all, of the following positions: 100e, 100h, 100j, and 101 (Kabat numbering) preferably wherein the ISVD comprises at least one, preferably all of the following amino acid residues at the following positions: I100e, Y100h, Y100j, and D101 (Kabat numbering).
22. The ISVD according to any one of the preceding items, wherein the ISVD comprises at least one, at least two, at least three, preferably all of the following amino acid residues at the following positions (Kabat numbering): T99, V100, I100e, M100g, Y100h, Y100j, D101, and Y102, preferably at least I100e, Y100h, Y100j, and D101.
23. The ISVD according to any one of the preceding items, wherein the CDR3 (AbM definition) consists of the following amino acid sequence X₁X₂STTVRX₃X₄CX₅IMYX₆YDY (SEQ ID NO: 4702), wherein:
   X₁ is selected from any amino acid, preferably X₁ is selected from A or G;
   X₂ is selected from any amino acid, preferably X₂ is selected T, A, E, G, F, I, D, H, K, S, P, R, Q, Y, V, or L;
   X₃ is selected from any amino acid, preferably X₃ is selected from D or G;
   X₄ is selected from any amino acid, preferably X₄ is selected from L, M, I, E, A, V, D, H, G, F, K, R, P, S, T, or Y;
   X₅ is selected from any amino acid, preferably X₅ is selected from G or F; and
   X₆ is selected from any amino acid, preferably X₆ is selected from L, V, or F.
24. The ISVD according to any one of the preceding items, wherein the following amino acid residue of the ISVD at the following position: R45 (Kabat numbering) is capable of forming salt bridges with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C.
25. The ISVD according to any one of the preceding items, wherein at least one, preferably all of the following amino acid residues of the ISVD at the following positions: I100e, Y100h, Y100j, and D101 (Kabat numbering) are capable of forming hydrogen bonds with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C.
26. The ISVD according to any one of the preceding items, wherein at least one, preferably all, of the following amino acid residues of the ISVD at the following positions: R45, I100e, Y100h, Y100j, and D101, and optionally Q105 (Kabat numbering) is capable of interacting with at least one, preferably all of the following amino acid residues of human PD-1 protein, when numbered in accordance with SEQ ID NO: 4703: N66, Y68, K78, A132, and E136, and optionally N74, preferably wherein:
   R45 in the ISVD (Kabat numbering) interacts, preferably by forming a salt bridge, with E136 in human PD-1 protein;
   1100e in the ISVD (Kabat numbering) interacts, preferably by forming a hydrogen bond, with A132 in human PD-1 protein;
   Y100 h in the ISVD (Kabat numbering) interacts, preferably by forming a hydrogen bond, with N66 in human PD-1 protein;
   Y100j in the ISVD (Kabat numbering) interacts, preferably by forming a hydrogen bond, with N66 in human PD-1 protein;
   Y100j in the ISVD (Kabat numbering) interacts, preferably by forming a hydrogen bond, with Y68 in human PD-1 protein;
   Y100j in the ISVD (Kabat numbering) interacts, preferably by forming a hydrogen bond, with A132 in human PD-1 protein;
   D101 in the ISVD (Kabat numbering) interacts, preferably by forming a hydrogen bond, with N66 in human PD-1 protein; and/or
   D101 in the ISVD (Kabat numbering) interacts, preferably by forming a hydrogen bond, with K78 in human PD-1 protein;
preferably when the ISVD and human PD-1 protein are mixed in PBS1× buffer and crystallized by hanging drop vapor diffusion using 30% PEG3350, 50 mM TRIS, pH 8 at 19° C.
27. The ISVD according to item 26, wherein:
   Q105 in the ISVD (Kabat numbering) interacts, preferably by forming a hydrogen bond, with N74 in human PD-1 protein.
28. The ISVD according to any one of the preceding items, wherein the ISVD comprises at least one, at least two, at least three, preferably all of the following amino acid residues at the following positions (Kabat numbering): I100e, Y100h, Y100j, and D101.
29. The ISVD according to any one of the preceding items, wherein the ISVD comprises at least one, at least two, at least three, at least four, at least five, at least six, at least seven, preferably all of the following amino acid residues at the following positions (Kabat numbering): T99, V100, I100e, M100g, Y100h, Y100j, D101, and Y102.
30. The ISVD according to any one of the preceding items, wherein said ISVD essentially consists of 4 framework regions (FR1 to FR4, respectively) and 3 complementarity determining regions (CDR1 to CDR3 respectively), in which CDR3 (AbM definition) has an amino acid sequence selected from:
   a) the amino acid sequence of SEQ ID NO: 691;
   b) amino acid sequences that have at least 80% amino acid identity with the amino acid sequence of SEQ ID NO: 691;
   c) amino acid sequences that have 3, 2, or 1 amino acid difference with the amino acid sequence of SEQ ID NO: 691.
31. The ISVD according to item 30, wherein the ISVD comprises V2, Q3, L4, R45, W103 and/or Q105 (Kabat numbering).
32. The ISVD according to item 31, wherein the ISVD comprises R45 (Kabat numbering).
33. The ISVD according to item 31 or 32, wherein the ISVD comprises:
   R45 and Q105 (Kabat numbering);
   R45 and W103 (Kabat numbering); or
   R45, W103 and Q105 (Kabat numbering).
34. The ISVD according to any one of the preceding items, wherein the ISVD comprises at least one, at least two, at least three, at least four, at least five, at least six, at least seven, at least eight, at least nine, at least ten, at least eleven, at least twelve, at least thirteen, preferably all of the following amino acid residues at the following positions (Kabat numbering): V2, Q3, L4, R45, T99, V100, I100e, M100g, Y100h, Y100j, D101, Y102, W103, and Q105.
35. The ISVD according to any one of the preceding items, in which R45, I100e, Y100h, Y100j, and D101, and optionally Q105 (Kabat numbering) of the ISVD are capable of interacting with at least one of the following amino acids of human PD-1 protein, when numbered in accordance with SEQ ID NO: 4703: N66, Y68, K78, A132, E136, and optionally N74, preferably when the ISVD and human PD-1 protein are mixed in PBS1× buffer and crystallized by hanging drop vapor diffusion using 30% PEG3350, 50 mM TRIS, pH 8 at 19° C.
36. The ISVD according to any one of the preceding items, in which V2, Q3, L4, R45, I100e, Y100h, Y100j, D101, and Q105 (Kabat numbering) of the ISVD are capable of interacting with the following amino acids of human PD-1 protein, when numbered in accordance with SEQ ID NO: 4703: N66, Y68, S73, N74, Q75, K78, A132, and E136, preferably when the ISVD and human PD-1 protein are mixed in PBS1× buffer and crystallized by hanging drop vapor diffusion using 30% PEG3350, 50 mM TRIS, pH 8 at 19° C.
37. The ISVD according to any one of the preceding items, wherein two amino acids are considered to interact when the distance between them is 4.0 Å or less.
38. The ISVD according to any one of the preceding items, wherein the interacting amino acid residue(s) in the ISVD and in human PD-1 protein are identified by X-ray crystallography by:
   Mixing human PD-1 protein, and the ISVD in PBS1× buffer;
   Concentrating the protein complex to 10.6 mg/ml;
   Crystallizing by hanging drop vapor diffusion using 30% PEG3350, 50 mM TRIS, pH 8 at 19° C.;
   Collecting X-ray diffraction data;
   Processing the collected data, preferably as described in Example 13.
39. An immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD cross-blocks the binding to human PD-1 protein of a polypeptide having an amino acid sequence selected from SEQ ID NOs: 1-488, preferably wherein the ISVD cross-blocks the binding to human PD-1 protein of a polypeptide having an amino acid sequence selected from SEQ ID NO: 402, or wherein the binding of the ISVD to human PD-1 protein is cross-blocked by a polypeptide having an amino acid sequence selected from SEQ ID NOs: 1-488.
40. The ISVD according to any one of the preceding items, wherein the ISVD competes with a polypeptide with an amino acid sequence selected from SEQ ID NO: 1-488 for binding human PD-1 protein, as measured in a competition assay, such as a FACS competition assay.
41. An immunoglobulin single variable domain (ISVD) specifically binding human PD-1, that essentially consists of 4 framework regions (FR1 to FR4, respectively) and 3 complementarity determining regions (CDR1 to CDR3, respectively), wherein CDR3 (AbM definition) consists of the amino acid sequence of one of SEQ ID NOs: 644-711, and wherein the amino acid residue of the ISVD at the following position: 45 (Kabat numbering) is capable of forming salt bridges with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C., preferably wherein the ISVD comprises at least the following amino acid residue: R45 (Kabat numbering).
42. The ISVD according to item 41, wherein R45 (Kabat numbering) of the ISVD is capable of forming salt bridges with the amino acid residue E136 of human PD-1 protein, when numbered in accordance with SEQ ID NO: 4703.
43. The ISVD according to item 41 or item 42, wherein the CDR3 (AbM definition) consists of the amino acid sequence of SEQ ID NO: 691.
44. An immunoglobulin single variable domain (ISVD) specifically binding human PD-1, that essentially consists of 4 framework regions (FR1 to FR4, respectively) and 3 complementarity determining regions (CDR1 to CDR3, respectively), in which:
   CDR1 (AbM definition) has an amino acid sequence selected from:
      a) the amino acid sequence of GFTLDX₁YX₂X₃G (SEQ ID NO: 2394), wherein X₁ is selected from A, D, Q, P, S, G, T, V, I, R, K, E, L, H, F, or Y, X₂ is selected from V or F, and X₃ is selected from I, E, D, A, F, G, H, L, K, T, M, S, P, R, Y, V, or Q;
      b) amino acid sequences that have at least 80% amino acid identity with the amino acid sequence of GFTLDX₁YX₂X₃G (SEQ ID NO: 2394), wherein X₁ is selected from A, D, Q, P, S, G, T, V, I, R, K, E, L, H, F, or Y, X₂ is selected from V or F, and X₃ is selected from I, E, D, A, F, G, H, L, K, T, M, S, P, R, Y, V, or Q; and
      c) amino acid sequences that have 3, 2, or 1 amino acid difference with the amino acid sequences of GFTLDX₁YX₂X₃G (SEQ ID NO: 2394), wherein X₁ is selected from A, D, Q, P, S, G, T, V, I, R, K, E, L, H, F, or Y, X₂ is selected from V or F, and X₃ is selected from I, E, D, A, F, G, H, L, K, T, M, S, P, R, Y, V, or Q;
and
   CDR2 (AbM definition) has an amino acid sequence selected from:
      a) the amino acid sequence of CX₁SX₂X₃X₄DRX₅X₆ (SEQ ID NO: 2395), wherein X₁ is selected from L, G, H, K, P, R, S, or T, X₂ is selected from S, E, G, H, P, or Q, and X₃ is selected from N, E, A, D, F, G, H, I, K, L, P, Q, R, S, T, V, or Y, X₄ is selected from G, D, F, H, K, L, P, Q, R, S, T, V, A, or Y, X₅ is selected from I, A, E, G, H, K, P, R, S, or T, and X₆ is selected from N, A, D, E, F, G, H, I, K, L, P, Q, R, S, T, V, or Y;
      b) amino acid sequences that have at least 80% amino acid identity with the amino acid sequence of CX₁SX₂X₃X₄DRX₅X₆ (SEQ ID NO: 2395), wherein X₁ is selected from L, G, H, K, P, R, S, or T, X₂ is selected from S, E, G, H, P, or Q, and X₃ is selected from N, E, A, D, F, G, H, I, K, L, P, Q, R, S, T, V, or Y, X₄ is selected from G, D, F, H, K, L, P, Q, R, S, T, V, A, or Y, X₅ is selected from I, A, E, G, H, K, P, R, S, or T, and X₆ is selected from N, A, D, E, F, G, H, I, K, L, P, Q, R, S, T, V, or Y;
      c) amino acid sequences that have 3, 2, or 1 amino acid difference with the amino acid sequence of CX₁SX₂X₃X₄DRX₅X₆ (SEQ ID NO: 2395), wherein X₁ is selected from L, G, H, K, P, R, S, or T, X₂ is selected from S, E, G, H, P, or Q, and X₃ is selected from N, E, A, D, F, G, H, I, K, L, P, Q, R, S, T, V, or Y, X₄ is selected from G, D, F, H, K, L, P, Q, R, S, T, V, A, or Y, X₅ is selected from I, A, E, G, H, K, P, R, S, or T, and X₆ is selected from N, A, D, E, F, G, H, I, K, L, P, Q, R, S, T, V, or Y;
and
   CDR3 (AbM definition) has an amino acid sequence selected from:
      a) the amino acid sequence of X₁X₂STX₃VRX₄X₅CX₆IX₇YX₈YDX₉ (SEQ ID NO: 2396), wherein X₁ is selected from A or G, X₂ is selected from T, A, E, G, F, I, D, H, K, S, P, R, Q, Y, V, or L, X₃ is selected from T or I, X₄ is selected from D or G, X₅ is selected from L, M, I, E, A, V, D, H, G, F, L, K, R, P, S, T, or Y, X₆ is selected from G or F, X₇ is selected from I, M, F, Y, L, A, D, E, G, K, H, P, Q, S, T, V, or R, X₈ is selected from L, V, or F, and X₉ is selected from Y, E, I, L, or V;
      b) amino acid sequences that have at least 80% amino acid identity with the amino acid sequence of X₁X₂STX₃VRX₄X₅CX₆IX₇YX₈YDX₉ (SEQ ID NO: 2396), wherein X₁ is selected from A or G, X₂ is selected from T, A, E, G, F, I, D, H, K, S, P, R, Q, Y, V, or L, X₃ is selected from T or I, X₄ is selected from D or G, X₅ is selected from L, M, I, E, A, V, D, H, G, F, L, K, R, P, S, T, or Y, X₆ is selected from G or F, X₇ is selected from I, M, F, Y, L, A, D, E, G, K, H, P, Q, S, T, V, or R, X₈ is selected from L, V, or F, and X₉ is selected from Y, E, I, L, or V;
      c) amino acid sequences that have 3, 2, or 1 amino acid difference with the amino acid sequence of X₁X₂STX₃VRX₄X₅CX₆IX₇YX₈YDX₉ (SEQ ID NO: 2396), wherein X₁ is selected from A or G, X₂ is selected from T, A, E, G, F, I, D, H, K, S, P, R, Q, Y, V, or L, X₃ is selected from T or I, X₄ is selected from D or G, X₅ is selected from L, M, I, E, A, V, D, H, G, F, L, K, R, P, S, T, or Y, X₆ is selected from G or F, X₇ is selected from I, M, F, Y, L, A, D, E, G, K, H, P, Q, S, T, V, or R, X₈ is selected from L, V, or F, and X₉ is selected from Y, E, I, L, or V.
45. The ISVD according to item 44, in which the amino acid sequences of the CDRs (AbM definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 1-488.
46. The ISVD according to any one of item 44 or 45, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of SEQ ID NO: 493;
      b) amino acid sequences that have at least 80% amino acid identity with the amino acid sequence of SEQ ID NO: 493; and
      c) amino acid sequences that have 3, 2, or 1 amino acid difference with the amino acid sequences of SEQ ID NO: 493;
and
   CDR2 (AbM definition) has an amino acid sequence selected from:
      a) the amino acid sequence of SEQ ID NO: 526;
      b) amino acid sequences that have at least 80% amino acid identity with the amino acid sequence of SEQ ID NO: 526;
      c) amino acid sequences that have 3, 2, or 1 amino acid difference with the amino acid sequence of SEQ ID NO: 526;
and
   CDR3 (AbM definition) has an amino acid sequence selected from:
      a) the amino acid sequence of SEQ ID NOs: 691;
      b) amino acid sequences that have at least 80% amino acid identity with the amino acid sequence of SEQ ID NO: 691;
      c) amino acid sequences that have 3, 2, or 1 amino acid difference with the amino acid sequence of SEQ ID NO: 691.
47. The ISVD according to any one of items 44 or 45, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of SEQ ID NO: 493;
      b) amino acid sequences that have at least 80% amino acid identity with the amino acid sequence of SEQ ID NO: 493; and
      c) amino acid sequences that have 3, 2, or 1 amino acid difference with the amino acid sequences of SEQ ID NO: 493;
and
   CDR2 (AbM definition) has an amino acid sequence selected from:
      a) the amino acid sequence of SEQ ID NO: 543;
      b) amino acid sequences that have at least 80% amino acid identity with the amino acid sequence of SEQ ID NO: 543;
      c) amino acid sequences that have 3, 2, or 1 amino acid difference with the amino acid sequence of SEQ ID NO: 543;
and
   CDR3 (AbM definition) has an amino acid sequence selected from:
      a) the amino acid sequence of SEQ ID NOs: 680;
      b) amino acid sequences that have at least 80% amino acid identity with the amino acid sequence of SEQ ID NO: 680;
      c) amino acid sequences that have 3, 2, or 1 amino acid difference with the amino acid sequence of SEQ ID NO: 680.
48. The ISVD according to any one of items 44 or 45, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of SEQ ID NO: 493;
      b) amino acid sequences that have at least 80% amino acid identity with the amino acid sequence of SEQ ID NO: 493; and
      c) amino acid sequences that have 3, 2, or 1 amino acid difference with the amino acid sequences of SEQ ID NO: 493;
and
   CDR2 (AbM definition) has an amino acid sequence selected from:
      a) the amino acid sequence of SEQ ID NO: 594;
      b) amino acid sequences that have at least 80% amino acid identity with the amino acid sequence of SEQ ID NO: 594;
      c) amino acid sequences that have 3, 2, or 1 amino acid difference with the amino acid sequence of SEQ ID NO: 594;
and
   CDR3 (AbM definition) has an amino acid sequence selected from:
      a) the amino acid sequence of SEQ ID NOs: 594;
      b) amino acid sequences that have at least 80% amino acid identity with the amino acid sequence of SEQ ID NOs: 594;
      c) amino acid sequences that have 3, 2, or 1 amino acid difference with the amino acid sequence of SEQ ID NO: 594.
49. The ISVD according to any one of items 44 or 45, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of SEQ ID NO: 493;
      b) amino acid sequences that have at least 80% amino acid identity with the amino acid sequence of SEQ ID NO: 493; and
      c) amino acid sequences that have 3, 2, or 1 amino acid difference with the amino acid sequences of SEQ ID NO: 493;
and
   CDR2 (AbM definition) has an amino acid sequence selected from:
      a) the amino acid sequence of SEQ ID NO: 593;
      b) amino acid sequences that have at least 80% amino acid identity with the amino acid sequence of SEQ ID NO: 593;
      c) amino acid sequences that have 3, 2, or 1 amino acid difference with the amino acid sequence of SEQ ID NO: 593;
and
   CDR3 (AbM definition) has an amino acid sequence selected from:
      a) the amino acid sequence of SEQ ID NOs: 701;
      b) amino acid sequences that have at least 80% amino acid identity with the amino acid sequence of SEQ ID NO: 701;
      c) amino acid sequences that have 3, 2, or 1 amino acid difference with the amino acid sequence of SEQ ID NO: 701.
50. The ISVD according to any one of items 44 or 45, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of SEQ ID NO: 493;
      b) amino acid sequences that have at least 80% amino acid identity with the amino acid sequence of SEQ ID NO: 493; and
      c) amino acid sequences that have 3, 2, or 1 amino acid difference with the amino acid sequences of SEQ ID NO: 493;
and
   CDR2 (AbM definition) has an amino acid sequence selected from:
      a) the amino acid sequence of SEQ ID NO: 549;
      b) amino acid sequences that have at least 80% amino acid identity with the amino acid sequence of SEQ ID NO: 549;
      c) amino acid sequences that have 3, 2, or 1 amino acid difference with the amino acid sequence of SEQ ID NO: 549;
and
   CDR3 (AbM definition) has an amino acid sequence selected from:
      a) the amino acid sequence of SEQ ID NO: 680;
      b) amino acid sequences that have at least 80% amino acid identity with the amino acid sequence of SEQ ID NO: 680;
      c) amino acid sequences that have 3, 2, or 1 amino acid difference with the amino acid sequence of SEQ ID NO: 680.
51. The ISVD according to any one of items 46 to 50 in which the amino acid sequences of the CDRs (AbM numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 402, 331, 332, 375, 212, 139, 238.
52. The ISVD according to any one of items 44 to 51, in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 546, CDR3 (AbM numbering) consists of the amino acid sequence of SEQ ID NO: 680; or in which
   CDR1 (AbM numbering) consists of the amino acid sequence of SEQ ID NO: 494, CDR2 (AbM numbering) consists of the amino acid sequence of SEQ ID NO: 547, CDR3 (AbM numbering) consists of the amino acid sequence of SEQ ID NO: 674; or in which
CDR1 (AbM numbering) consists of the amino acid sequence of SEQ ID NO: 495, CDR2 (AbM numbering) consists of the amino acid sequence of SEQ ID NO: 547, CDR3 (AbM numbering) consists of the amino acid sequence of SEQ ID NO: 674; or in which
   CDR1 (AbM numbering) consists of the amino acid sequence of SEQ ID NO: 496, CDR2 (AbM numbering) consists of the amino acid sequence of SEQ ID NO: 547, CDR3 (AbM numbering) consists of the amino acid sequence of SEQ ID NO: 674; or in which
   CDR1 (AbM numbering) consists of the amino acid sequence of SEQ ID NO: 493, CDR2 (AbM numbering) consists of the amino acid sequence of SEQ ID NO: 547, CDR3 (AbM numbering) consists of the amino acid sequence of SEQ ID NO: 674; or in which
   CDR1 (AbM numbering) consists of the amino acid sequence of SEQ ID NO: 494, CDR2 (AbM numbering) consists of the amino acid sequence of SEQ ID NO: 541, CDR3 (AbM numbering) consists of the amino acid sequence of SEQ ID NO: 674; or in which
   CDR1 (AbM numbering) consists of the amino acid sequence of SEQ ID NO: 495, CDR2 (AbM numbering) consists of the amino acid sequence of SEQ ID NO: 541, CDR3 (AbM numbering) consists of the amino acid sequence of SEQ ID NO: 674; or in which
   CDR1 (AbM numbering) consists of the amino acid sequence of SEQ ID NO: 496, CDR2 (AbM numbering) consists of the amino acid sequence of SEQ ID NO: 541, CDR3 (AbM numbering) consists of the amino acid sequence of SEQ ID NO: 674; or in which
   CDR1 (AbM numbering) consists of the amino acid sequence of SEQ ID NO: 493, CDR2 (AbM numbering) consists of the amino acid sequence of SEQ ID NO: 541, CDR3 (AbM numbering) consists of the amino acid sequence of SEQ ID NO: 674; or in which
   CDR1 (AbM numbering) consists of the amino acid sequence of SEQ ID NO: 494, CDR2 (AbM numbering) consists of the amino acid sequence of SEQ ID NO: 549, CDR3 (AbM numbering) consists of the amino acid sequence of SEQ ID NO: 674; or in which
   CDR1 (AbM numbering) consists of the amino acid sequence of SEQ ID NO: 495, CDR2 (AbM numbering) consists of the amino acid sequence of SEQ ID NO: 549, CDR3 (AbM numbering) consists of the amino acid sequence of SEQ ID NO: 674; or in which
   CDR1 (AbM numbering) consists of the amino acid sequence of SEQ ID NO: 496, CDR2 (AbM numbering) consists of the amino acid sequence of SEQ ID NO: 549, CDR3 (AbM numbering) consists of the amino acid sequence of SEQ ID NO: 674; or in which
   CDR1 (AbM numbering) consists of the amino acid sequence of SEQ ID NO: 493, CDR2 (AbM numbering) consists of the amino acid sequence of SEQ ID NO: 548, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 549, CDR3 (AbM numbering) consists of the amino acid sequence of SEQ ID NO: 674; or in which
   CDR1 (AbM numbering) consists of the amino acid sequence of SEQ ID NO: 494, CDR2 (AbM numbering) consists of the amino acid sequence of SEQ ID NO: 547, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 540, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 542, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 544, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 530, CDR3 (AbM numbering) consists of the amino acid sequence of SEQ ID NO: 680; or in which
   CDR1 (AbM numbering) consists of the amino acid sequence of SEQ ID NO: 495, CDR2 (AbM numbering) consists of the amino acid sequence of SEQ ID NO: 604, CDR3 (AbM numbering) consists of the amino acid sequence of SEQ ID NO: 680; or in which
   CDR1 (AbM numbering) consists of the amino acid sequence of SEQ ID NO: 496, CDR2 (AbM numbering) consists of the amino acid sequence of SEQ ID NO: 604, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 604, CDR3 (AbM numbering) consists of the amino acid sequence of SEQ ID NO: 680; or in which
   CDR1 (AbM numbering) consists of the amino acid sequence of SEQ ID NO: 494, CDR2 (AbM numbering) consists of the amino acid sequence of SEQ ID NO: 603, CDR3 (AbM numbering) consists of the amino acid sequence of SEQ ID NO: 680; or in which
   CDR1 (AbM numbering) consists of the amino acid sequence of SEQ ID NO: 495, CDR2 (AbM numbering) consists of the amino acid sequence of SEQ ID NO: 603, CDR3 (AbM numbering) consists of the amino acid sequence of SEQ ID NO: 680; or in which
   CDR1 (AbM numbering) consists of the amino acid sequence of SEQ ID NO: 496, CDR2 (AbM numbering) consists of the amino acid sequence of SEQ ID NO: 603, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 603, CDR3 (AbM numbering) consists of the amino acid sequence of SEQ ID NO: 680; or in which
   CDR1 (AbM numbering) consists of the amino acid sequence of SEQ ID NO: 494, CDR2 (AbM numbering) consists of the amino acid sequence of SEQ ID NO: 543, CDR3 (AbM numbering) consists of the amino acid sequence of SEQ ID NO: 680; or in which
   CDR1 (AbM numbering) consists of the amino acid sequence of SEQ ID NO: 496, CDR2 (AbM numbering) consists of the amino acid sequence of SEQ ID NO: 543, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 680; or in which
   CDR1 (AbM numbering) consists of the amino acid sequence of SEQ ID NO: 494, CDR2 (AbM numbering) consists of the amino acid sequence of SEQ ID NO: 604, CDR3 (AbM numbering) consists of the amino acid sequence of SEQ ID NO: 680; or in which
   CDR1 (AbM numbering) consists of the amino acid sequence of SEQ ID NO: 495, CDR2 (AbM numbering) consists of the amino acid sequence of SEQ ID NO: 543, CDR3 (AbM numbering) consists of the amino acid sequence of SEQ ID NO: 680; or in which
   CDR1 (AbM numbering) consists of the amino acid sequence of SEQ ID NO: 495, CDR2 (AbM numbering) consists of the amino acid sequence of SEQ ID NO: 547, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 535, CDR3 (AbM numbering) consists of the amino acid sequence of SEQ ID NO: 680; or in which
   CDR1 (AbM numbering) consists of the amino acid sequence of SEQ ID NO: 496, CDR2 (AbM numbering) consists of the amino acid sequence of SEQ ID NO: 547, CDR3 (AbM numbering) consists of the amino acid sequence of SEQ ID NO: 680; or in which
   CDR1 (AbM numbering) consists of the amino acid sequence of SEQ ID NO: 494, CDR2 (AbM numbering) consists of the amino acid sequence of SEQ ID NO: 541, CDR3 (AbM numbering) consists of the amino acid sequence of SEQ ID NO: 680; or in which
   CDR1 (AbM numbering) consists of the amino acid sequence of SEQ ID NO: 496, CDR2 (AbM numbering) consists of the amino acid sequence of SEQ ID NO: 531, CDR3 (AbM numbering) consists of the amino acid sequence of SEQ ID NO: 674; or in which
   CDR1 (AbM numbering) consists of the amino acid sequence of SEQ ID NO: 493, CDR2 (AbM numbering) consists of the amino acid sequence of SEQ ID NO: 531, CDR3 (AbM numbering) consists of the amino acid sequence of SEQ ID NO: 674; or in which
   CDR1 (AbM numbering) consists of the amino acid sequence of SEQ ID NO: 494, CDR2 (AbM numbering) consists of the amino acid sequence of SEQ ID NO: 534, CDR3 (AbM numbering) consists of the amino acid sequence of SEQ ID NO: 680; or in which
   CDR1 (AbM numbering) consists of the amino acid sequence of SEQ ID NO: 495, CDR2 (AbM numbering) consists of the amino acid sequence of SEQ ID NO: 534, CDR3 (AbM numbering) consists of the amino acid sequence of SEQ ID NO: 680; or in which
   CDR1 (AbM numbering) consists of the amino acid sequence of SEQ ID NO: 496, CDR2 (AbM numbering) consists of the amino acid sequence of SEQ ID NO: 534, CDR3 (AbM numbering) consists of the amino acid sequence of SEQ ID NO: 680; or in which
   CDR1 (AbM numbering) consists of the amino acid sequence of SEQ ID NO: 494, CDR2 (AbM numbering) consists of the amino acid sequence of SEQ ID NO: 531, CDR3 (AbM numbering) consists of the amino acid sequence of SEQ ID NO: 680; or in which
   CDR1 (AbM numbering) consists of the amino acid sequence of SEQ ID NO: 495, CDR2 (AbM numbering) consists of the amino acid sequence of SEQ ID NO: 531, CDR3 (AbM numbering) consists of the amino acid sequence of SEQ ID NO: 680; or in which
   CDR1 (AbM numbering) consists of the amino acid sequence of SEQ ID NO: 496, CDR2 (AbM numbering) consists of the amino acid sequence of SEQ ID NO: 531, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 531, CDR3 (AbM numbering) consists of the amino acid sequence of SEQ ID NO: 680; or in which
   CDR1 (AbM numbering) consists of the amino acid sequence of SEQ ID NO: 495, CDR2 (AbM numbering) consists of the amino acid sequence of SEQ ID NO: 531, CDR3 (AbM numbering) consists of the amino acid sequence of SEQ ID NO: 674; or in which
   CDR1 (AbM numbering) consists of the amino acid sequence of SEQ ID NO: 493, CDR2 (AbM numbering) consists of the amino acid sequence of SEQ ID NO: 529, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 602, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 527, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 528, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 601, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 532, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 534, CDR3 (AbM numbering) consists of the amino acid sequence of SEQ ID NO: 680; or in which
   CDR1 (AbM numbering) consists of the amino acid sequence of SEQ ID NO: 494, CDR2 (AbM numbering) consists of the amino acid sequence of SEQ ID NO: 531, CDR3 (AbM numbering) consists of the amino acid sequence of SEQ ID NO: 674; or in which
   CDR1 (AbM numbering) consists of the amino acid sequence of SEQ ID NO: 493, CDR2 (AbM numbering) consists of the amino acid sequence of SEQ ID NO: 534, CDR3 (AbM numbering) consists of the amino acid sequence of SEQ ID NO: 674; or in which
   CDR1 (AbM numbering) consists of the amino acid sequence of SEQ ID NO: 496, CDR2 (AbM numbering) consists of the amino acid sequence of SEQ ID NO: 534, CDR3 (AbM numbering) consists of the amino acid sequence of SEQ ID NO: 674; or in which
   CDR1 (AbM numbering) consists of the amino acid sequence of SEQ ID NO: 495, CDR2 (AbM numbering) consists of the amino acid sequence of SEQ ID NO: 541, CDR3 (AbM numbering) consists of the amino acid sequence of SEQ ID NO: 680; or in which
   CDR1 (AbM numbering) consists of the amino acid sequence of SEQ ID NO: 496, CDR2 (AbM numbering) consists of the amino acid sequence of SEQ ID NO: 541, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 541, CDR3 (AbM numbering) consists of the amino acid sequence of SEQ ID NO: 680; or in which
   CDR1 (AbM numbering) consists of the amino acid sequence of SEQ ID NO: 494, CDR2 (AbM numbering) consists of the amino acid sequence of SEQ ID NO: 549, CDR3 (AbM numbering) consists of the amino acid sequence of SEQ ID NO: 680; or in which
   CDR1 (AbM numbering) consists of the amino acid sequence of SEQ ID NO: 496, CDR2 (AbM numbering) consists of the amino acid sequence of SEQ ID NO: 549, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 549, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 547, CDR3 (AbM numbering) consists of the amino acid sequence of SEQ ID NO: 680; or in which
   CDR1 (AbM numbering) consists of the amino acid sequence of SEQ ID NO: 495, CDR2 (AbM numbering) consists of the amino acid sequence of SEQ ID NO: 549, CDR3 (AbM numbering) consists of the amino acid sequence of SEQ ID NO: 680; or in which
   CDR1 (AbM numbering) consists of the amino acid sequence of SEQ ID NO: 494, CDR2 (AbM numbering) consists of the amino acid sequence of SEQ ID NO: 534, CDR3 (AbM numbering) consists of the amino acid sequence of SEQ ID NO: 674; or in which
   CDR1 (AbM numbering) consists of the amino acid sequence of SEQ ID NO: 495, CDR2 (AbM numbering) consists of the amino acid sequence of SEQ ID NO: 534, CDR3 (AbM numbering) consists of the amino acid sequence of SEQ ID NO: 674; or in which
   CDR1 (AbM numbering) consists of the amino acid sequence of SEQ ID NO: 494, CDR2 (AbM numbering) consists of the amino acid sequence of SEQ ID NO: 543, CDR3 (AbM numbering) consists of the amino acid sequence of SEQ ID NO: 674; or in which
   CDR1 (AbM numbering) consists of the amino acid sequence of SEQ ID NO: 493, CDR2 (AbM numbering) consists of the amino acid sequence of SEQ ID NO: 578, CDR3 (AbM numbering) consists of the amino acid sequence of SEQ ID NO: 707; or in which
   CDR1 (AbM numbering) consists of the amino acid sequence of SEQ ID NO: 493, CDR2 (AbM numbering) consists of the amino acid sequence of SEQ ID NO: 578, CDR3 (AbM numbering) consists of the amino acid sequence of SEQ ID NO: 703; or in which
   CDR1 (AbM numbering) consists of the amino acid sequence of SEQ ID NO: 493, CDR2 (AbM numbering) consists of the amino acid sequence of SEQ ID NO: 578, CDR3 (AbM numbering) consists of the amino acid sequence of SEQ ID NO: 672; or in which
   CDR1 (AbM numbering) consists of the amino acid sequence of SEQ ID NO: 493, CDR2 (AbM numbering) consists of the amino acid sequence of SEQ ID NO: 578, CDR3 (AbM numbering) consists of the amino acid sequence of SEQ ID NO: 665; or in which
   CDR1 (AbM numbering) consists of the amino acid sequence of SEQ ID NO: 493, CDR2 (AbM numbering) consists of the amino acid sequence of SEQ ID NO: 592, CDR3 (AbM numbering) consists of the amino acid sequence of SEQ ID NO: 701; or in which
   CDR1 (AbM numbering) consists of the amino acid sequence of SEQ ID NO: 493, CDR2 (AbM numbering) consists of the amino acid sequence of SEQ ID NO: 592, CDR3 (AbM numbering) consists of the amino acid sequence of SEQ ID NO: 703; or in which
   CDR1 (AbM numbering) consists of the amino acid sequence of SEQ ID NO: 493, CDR2 (AbM numbering) consists of the amino acid sequence of SEQ ID NO: 592, CDR3 (AbM numbering) consists of the amino acid sequence of SEQ ID NO: 672; or in which
   CDR1 (AbM numbering) consists of the amino acid sequence of SEQ ID NO: 493, CDR2 (AbM numbering) consists of the amino acid sequence of SEQ ID NO: 592, CDR3 (AbM numbering) consists of the amino acid sequence of SEQ ID NO: 665; or in which
   CDR1 (AbM numbering) consists of the amino acid sequence of SEQ ID NO: 493, CDR2 (AbM numbering) consists of the amino acid sequence of SEQ ID NO: 592, CDR3 (AbM numbering) consists of the amino acid sequence of SEQ ID NO: 707; or in which
   CDR1 (AbM numbering) consists of the amino acid sequence of SEQ ID NO: 493, CDR2 (AbM numbering) consists of the amino acid sequence of SEQ ID NO: 593, CDR3 (AbM numbering) consists of the amino acid sequence of SEQ ID NO: 701; or in which
   CDR1 (AbM numbering) consists of the amino acid sequence of SEQ ID NO: 493, CDR2 (AbM numbering) consists of the amino acid sequence of SEQ ID NO: 593, CDR3 (AbM numbering) consists of the amino acid sequence of SEQ ID NO: 703; or in which
   CDR1 (AbM numbering) consists of the amino acid sequence of SEQ ID NO: 493, CDR2 (AbM numbering) consists of the amino acid sequence of SEQ ID NO: 593, CDR3 (AbM numbering) consists of the amino acid sequence of SEQ ID NO: 672; or in which
   CDR1 (AbM numbering) consists of the amino acid sequence of SEQ ID NO: 493, CDR2 (AbM numbering) consists of the amino acid sequence of SEQ ID NO: 593, CDR3 (AbM numbering) consists of the amino acid sequence of SEQ ID NO: 665; or in which
   CDR1 (AbM numbering) consists of the amino acid sequence of SEQ ID NO: 493, CDR2 (AbM numbering) consists of the amino acid sequence of SEQ ID NO: 593, CDR3 (AbM numbering) consists of the amino acid sequence of SEQ ID NO: 707; or in which
   CDR1 (AbM numbering) consists of the amino acid sequence of SEQ ID NO: 493, CDR2 (AbM numbering) consists of the amino acid sequence of SEQ ID NO: 594, CDR3 (AbM numbering) consists of the amino acid sequence of SEQ ID NO: 701; or in which
   CDR1 (AbM numbering) consists of the amino acid sequence of SEQ ID NO: 493, CDR2 (AbM numbering) consists of the amino acid sequence of SEQ ID NO: 594, CDR3 (AbM numbering) consists of the amino acid sequence of SEQ ID NO: 703; or in which
   CDR1 (AbM numbering) consists of the amino acid sequence of SEQ ID NO: 494, CDR2 (AbM numbering) consists of the amino acid sequence of SEQ ID NO: 537, CDR3 (AbM numbering) consists of the amino acid sequence of SEQ ID NO: 670; or in which
   CDR1 (AbM numbering) consists of the amino acid sequence of SEQ ID NO: 494, CDR2 (AbM numbering) consists of the amino acid sequence of SEQ ID NO: 593, CDR3 (AbM numbering) consists of the amino acid sequence of SEQ ID NO: 670; or in which
   CDR1 (AbM numbering) consists of the amino acid sequence of SEQ ID NO: 493, CDR2 (AbM numbering) consists of the amino acid sequence of SEQ ID NO: 559, CDR3 (AbM numbering) consists of the amino acid sequence of SEQ ID NO: 645; or in which
   CDR1 (AbM numbering) consists of the amino acid sequence of SEQ ID NO: 494, CDR2 (AbM numbering) consists of the amino acid sequence of SEQ ID NO: 553, CDR3 (AbM numbering) consists of the amino acid sequence of SEQ ID NO: 670; or in which
   CDR1 (AbM numbering) consists of the amino acid sequence of SEQ ID NO: 493, CDR2 (AbM numbering) consists of the amino acid sequence of SEQ ID NO: 598, CDR3 (AbM numbering) consists of the amino acid sequence of SEQ ID NO: 645; or in which
   CDR1 (AbM numbering) consists of the amino acid sequence of SEQ ID NO: 493, CDR2 (AbM numbering) consists of the amino acid sequence of SEQ ID NO: 593, CDR3 (AbM numbering) consists of the amino acid sequence of SEQ ID NO: 644; or in which
   CDR1 (AbM numbering) consists of the amino acid sequence of SEQ ID NO: 493, CDR2 (AbM numbering) consists of the amino acid sequence of SEQ ID NO: 557, CDR3 (AbM numbering) consists of the amino acid sequence of SEQ ID NO: 645; or in which
   CDR1 (AbM numbering) consists of the amino acid sequence of SEQ ID NO: 494, CDR2 (AbM numbering) consists of the amino acid sequence of SEQ ID NO: 550, CDR3 (AbM numbering) consists of the amino acid sequence of SEQ ID NO: 670; or in which
   CDR1 (AbM numbering) consists of the amino acid sequence of SEQ ID NO: 494, CDR2 (AbM numbering) consists of the amino acid sequence of SEQ ID NO: 595, CDR3 (AbM numbering) consists of the amino acid sequence of SEQ ID NO: 670; or in which
   CDR1 (AbM numbering) consists of the amino acid sequence of SEQ ID NO: 494, CDR2 (AbM numbering) consists of the amino acid sequence of SEQ ID NO: 555, CDR3 (AbM numbering) consists of the amino acid sequence of SEQ ID NO: 670; or in which
   CDR1 (AbM numbering) consists of the amino acid sequence of SEQ ID NO: 494, CDR2 (AbM numbering) consists of the amino acid sequence of SEQ ID NO: 559, CDR3 (AbM numbering) consists of the amino acid sequence of SEQ ID NO: 670; or in which
   CDR1 (AbM numbering) consists of the amino acid sequence of SEQ ID NO: 493, CDR2 (AbM numbering) consists of the amino acid sequence of SEQ ID NO: 550, CDR3 (AbM numbering) consists of the amino acid sequence of SEQ ID NO: 645; or in which
   CDR1 (AbM numbering) consists of the amino acid sequence of SEQ ID NO: 493, CDR2 (AbM numbering) consists of the amino acid sequence of SEQ ID NO: 553, CDR3 (AbM numbering) consists of the amino acid sequence of SEQ ID NO: 645; or in which
   CDR1 (AbM numbering) consists of the amino acid sequence of SEQ ID NO: 494, CDR2 (AbM numbering) consists of the amino acid sequence of SEQ ID NO: 591, CDR3 (AbM numbering) consists of the amino acid sequence of SEQ ID NO: 670; or in which
   CDR1 (AbM numbering) consists of the amino acid sequence of SEQ ID NO: 493, CDR2 (AbM numbering) consists of the amino acid sequence of SEQ ID NO: 578, CDR3 (AbM numbering) consists of the amino acid sequence of SEQ ID NO: 701; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 701; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 703; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 672; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 665; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 707; or in which
   CDR1 (AbM numbering) consists of the amino acid sequence of SEQ ID NO: 493, CDR2 (AbM numbering) consists of the amino acid sequence of SEQ ID NO: 594, CDR3 (AbM numbering) consists of the amino acid sequence of SEQ ID NO: 672; or in which
   CDR1 (AbM numbering) consists of the amino acid sequence of SEQ ID NO: 493, CDR2 (AbM numbering) consists of the amino acid sequence of SEQ ID NO: 603, CDR3 (AbM numbering) consists of the amino acid sequence of SEQ ID NO: 674; or in which
   CDR1 (AbM numbering) consists of the amino acid sequence of SEQ ID NO: 496, CDR2 (AbM numbering) consists of the amino acid sequence of SEQ ID NO: 543, CDR3 (AbM numbering) consists of the amino acid sequence of SEQ ID NO: 674; or in which
   CDR1 (AbM numbering) consists of the amino acid sequence of SEQ ID NO: 493, CDR2 (AbM numbering) consists of the amino acid sequence of SEQ ID NO: 539, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 678; or in which
   CDR1 (AbM numbering) consists of the amino acid sequence of SEQ ID NO: 494, CDR2 (AbM numbering) consists of the amino acid sequence of SEQ ID NO: 604, CDR3 (AbM numbering) consists of the amino acid sequence of SEQ ID NO: 674; or in which
   CDR1 (AbM numbering) consists of the amino acid sequence of SEQ ID NO: 495, CDR2 (AbM numbering) consists of the amino acid sequence of SEQ ID NO: 604, CDR3 (AbM numbering) consists of the amino acid sequence of SEQ ID NO: 674; or in which
   CDR1 (AbM numbering) consists of the amino acid sequence of SEQ ID NO: 496, CDR2 (AbM numbering) consists of the amino acid sequence of SEQ ID NO: 604, CDR3 (AbM numbering) consists of the amino acid sequence of SEQ ID NO: 674; or in which
   CDR1 (AbM numbering) consists of the amino acid sequence of SEQ ID NO: 493, CDR2 (AbM numbering) consists of the amino acid sequence of SEQ ID NO: 604, CDR3 (AbM numbering) consists of the amino acid sequence of SEQ ID NO: 674; or in which
   CDR1 (AbM numbering) consists of the amino acid sequence of SEQ ID NO: 494, CDR2 (AbM numbering) consists of the amino acid sequence of SEQ ID NO: 603, CDR3 (AbM numbering) consists of the amino acid sequence of SEQ ID NO: 674; or in which
   CDR1 (AbM numbering) consists of the amino acid sequence of SEQ ID NO: 495, CDR2 (AbM numbering) consists of the amino acid sequence of SEQ ID NO: 603, CDR3 (AbM numbering) consists of the amino acid sequence of SEQ ID NO: 674; or in which
   CDR1 (AbM numbering) consists of the amino acid sequence of SEQ ID NO: 495, CDR2 (AbM numbering) consists of the amino acid sequence of SEQ ID NO: 543, CDR3 (AbM numbering) consists of the amino acid sequence of SEQ ID NO: 674; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 674; or in which
   CDR1 (AbM numbering) consists of the amino acid sequence of SEQ ID NO: 496, CDR2 (AbM numbering) consists of the amino acid sequence of SEQ ID NO: 603, CDR3 (AbM numbering) consists of the amino acid sequence of SEQ ID NO: 674; or in which
   CDR1 (AbM numbering) consists of the amino acid sequence of SEQ ID NO: 493, CDR2 (AbM numbering) consists of the amino acid sequence of SEQ ID NO: 594, CDR3 (AbM numbering) consists of the amino acid sequence of SEQ ID NO: 707; or in which
   CDR1 (AbM numbering) consists of the amino acid sequence of SEQ ID NO: 493, CDR2 (AbM numbering) consists of the amino acid sequence of SEQ ID NO: 594, CDR3 (AbM numbering) consists of the amino acid sequence of SEQ ID NO: 665; or in which
   CDR1 (AbM numbering) consists of the amino acid sequence of SEQ ID NO: 493, CDR2 (AbM numbering) consists of the amino acid sequence of SEQ ID NO: 595, CDR3 (AbM numbering) consists of the amino acid sequence of SEQ ID NO: 701; or in which
   CDR1 (AbM numbering) consists of the amino acid sequence of SEQ ID NO: 493, CDR2 (AbM numbering) consists of the amino acid sequence of SEQ ID NO: 595, CDR3 (AbM numbering) consists of the amino acid sequence of SEQ ID NO: 703; or in which
   CDR1 (AbM numbering) consists of the amino acid sequence of SEQ ID NO: 493, CDR2 (AbM numbering) consists of the amino acid sequence of SEQ ID NO: 595, CDR3 (AbM numbering) consists of the amino acid sequence of SEQ ID NO: 672; or in which
   CDR1 (AbM numbering) consists of the amino acid sequence of SEQ ID NO: 493, CDR2 (AbM numbering) consists of the amino acid sequence of SEQ ID NO: 595, CDR3 (AbM numbering) consists of the amino acid sequence of SEQ ID NO: 665; or in which
   CDR1 (AbM numbering) consists of the amino acid sequence of SEQ ID NO: 493, CDR2 (AbM numbering) consists of the amino acid sequence of SEQ ID NO: 595, CDR3 (AbM numbering) consists of the amino acid sequence of SEQ ID NO: 707; or in which
   CDR1 (AbM numbering) consists of the amino acid sequence of SEQ ID NO: 494, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 494, CDR2 (AbM numbering) consists of the amino acid sequence of SEQ ID NO: 542, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 676; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 677; or in which
   CDR1 (AbM numbering) consists of the amino acid sequence of SEQ ID NO: 493, CDR2 (AbM numbering) consists of the amino acid sequence of SEQ ID NO: 575, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 570, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 566, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 564, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 567, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 572, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 580, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 587, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 588, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 583, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 568, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 584, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 560, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 586, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 2393, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 585, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 561, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 582, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 581, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 592, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 552, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 558, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 590, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 597, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 579, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 577, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 571, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 567, CDR3 (AbM numbering) consists of the amino acid sequence of SEQ ID NO: 690; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 702; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 667; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 673; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 663; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 666; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 661; or in which
   CDR1 (AbM numbering) consists of the amino acid sequence of SEQ ID NO: 493, CDR2 (AbM numbering) consists of the amino acid sequence of SEQ ID NO: 576, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 573, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 562, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 574, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 563, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 556, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 589, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 599, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 681; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 662; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 538, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 594, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 554, CDR3 (AbM numbering) consists of the amino acid sequence of SEQ ID NO: 691; or in which
   CDR1 (AbM numbering) consists of the amino acid sequence of SEQ ID NO: 497, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 498, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 499, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 500, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 501, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 502, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 503, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 504, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 505, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 506, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 507, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 508, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 509, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 510, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 511, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 536, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 700; or in which
   CDR1 (AbM numbering) consists of the amino acid sequence of SEQ ID NO: 493, CDR2 (AbM numbering) consists of the amino acid sequence of SEQ ID NO: 569, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 565, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 675; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 687; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 706; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 689; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 669; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 664; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 704; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 684; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 665; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 707; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 704; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 670; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 664; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 675; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 649; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 650; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 660; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 648; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 710; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 711; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 657; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 705; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 653; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 647; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 651; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 679; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 705; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 706; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 680; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 665; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 707; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 689; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 687; or in which
   CDR1 (AbM numbering) consists of the amino acid sequence of SEQ ID NO: 493, CDR2 (AbM numbering) consists of the amino acid sequence of SEQ ID NO: 551, CDR3 (AbM numbering) consists of the amino acid sequence of SEQ ID NO: 684; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 655; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 699; or in which
   CDR1 (AbM numbering) consists of the amino acid sequence of SEQ ID NO: 493, CDR2 (AbM numbering) consists of the amino acid sequence of SEQ ID NO: 543, CDR3 (AbM numbering) consists of the amino acid sequence of SEQ ID NO: 671; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 683; or in which
   CDR1 (AbM numbering) consists of the amino acid sequence of SEQ ID NO: 2392, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 512, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 513, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 692; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 694; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 689; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 688; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 695; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 682; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 687; or in which
   CDR1 (AbM numbering) consists of the amino acid sequence of SEQ ID NO: 494, CDR2 (AbM numbering) consists of the amino acid sequence of SEQ ID NO: 600, CDR3 (AbM numbering) consists of the amino acid sequence of SEQ ID NO: 670; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 696; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 685; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 686; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 697; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 698; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 684; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 708; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 709; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 668; or in which
   CDR1 (AbM numbering) consists of the amino acid sequence of SEQ ID NO: 494, CDR2 (AbM numbering) consists of the amino acid sequence of SEQ ID NO: 557, CDR3 (AbM numbering) consists of the amino acid sequence of SEQ ID NO: 670; or in which
   CDR1 (AbM numbering) consists of the amino acid sequence of SEQ ID NO: 514, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 693; or in which
   CDR1 (AbM numbering) consists of the amino acid sequence of SEQ ID NO: 496, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 646; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 652; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 659; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 658; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 656; or in which
   CDR1 (AbM numbering) consists of the amino acid sequence of SEQ ID NO: 493, CDR2 (AbM numbering) consists of the amino acid sequence of SEQ ID NO: 526, CDR3 (AbM numbering) consists of the amino acid sequence of SEQ ID NO: 654; or in which
   CDR1 (AbM numbering) consists of the amino acid sequence of SEQ ID NO: 515, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 516, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 517, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 518, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 519, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 520, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 495, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 521, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 522, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 523, CDR2 (AbM numbering) consists of the amino acid sequence of SEQ ID NO: 526, CDR3 (AbM numbering) consists of the amino acid sequence of SEQ ID NO: 691; or in which
   CDR1 (AbM numbering) consists of the amino acid sequence of SEQ ID NO: 494, CDR2 (AbM numbering) consists of the amino acid sequence of SEQ ID NO: 596, CDR3 (AbM numbering) consists of the amino acid sequence of SEQ ID NO: 670.
53. The ISVD according to any one of items 44 to 52, wherein the ISVD comprises V2, Q3, L4, R45, W103 and/or Q105 (Kabat numbering).
54. The ISVD according to item 53, wherein the ISVD comprises R45 (Kabat numbering).
55. The ISVD according to item 53 or 54, wherein the ISVD comprises:
   R45 and Q105 (Kabat numbering);
   R45 and W103 (Kabat numbering); or
   R45, W103 and Q105 (Kabat numbering).
56. The ISVD according to any one of items 44 to 52, wherein the ISVD comprises V2, Q3, L4, R45, W103 and Q105 (Kabat numbering).
57. The ISVD according to any one of items 44 to 56, which amino acid sequence
   iii) has 80% amino acid sequence identity with one of the amino acid sequences of SEQ ID NOs: 1-488, in which for the purposes of determining the degree of amino acid identity, the amino acid residues that form the CDR sequences are disregarded;
and in which:
   preferably one or more of the amino acid residues at positions 11, 37, 44, 45, 47, 83, 84, 103, 104 and 108 according to the Kabat numbering are chosen from the Hallmark residues mentioned in Table 1.
58. The ISVD according to any one of items 44 to 57, wherein the amino acid residue of the ISVD at the following position: 45 (Kabat numbering) is capable of forming salt bridges with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C. preferably wherein the ISVD comprises at least the following amino acid residue: R45 (Kabat numbering).
59. The ISVD according to item 58, wherein R45 (Kabat numbering) of the ISVD is capable of forming salt bridges with the amino acid residue E136 of human PD-1 protein, when numbered in accordance with SEQ ID NO: 4703,
60. The ISVD according to any one of items 1 to 59, that essentially consists of a heavy chain variable domain sequence that is derived from a conventional four-chain antibody or that essentially consists of a heavy chain variable domain sequence that is derived from a heavy chain antibody.
61. The ISVD according to any one of items 1 to 60, that essentially consists of a VHH, a humanized VHH, a camelized VH, a domain antibody, a single domain antibody, or a dAb.
62. The ISVD according to any one of items 1 to 61, which is a humanized ISVD that is chosen from SEQ ID NO: 1-401, 403-488 or from the group consisting of amino acid sequences that have more than 80%, preferably more than 90%, more preferably more than 95%, such as 99% or more amino acid sequence identity with at least one of the amino acid sequences of SEQ ID NOs: 1-488.
63. The ISVD according to any one of items 1 to 62, which is a humanized ISVD that is chosen from SEQ ID NO: 332, 375, 212, 139, and 238 or from the group consisting of amino acid sequences that have more than 80%, preferably more than 90%, more preferably more than 95%, such as 99% or more amino acid sequence identity with at least one of the amino acid sequences of SEQ ID NOs: 402, 332, 375, 212, 139, 238.
64. The ISVD according to any one of items 1 to 63, in which the amino acid sequence is chosen from the group consisting of SEQ ID NO: 402 and SEQ ID NOs: 1-401, 403-488.
65. The ISVD according to any one of items 1 to 64, wherein the ISVD specifically binds to human PD-1 with a dissociation constant (K_{D}) of 10⁻⁹ moles/litre or less, 10⁻¹⁰ moles/litre or less, such as 5×10″ 9 to 10⁻¹² moles/litre or less, and preferably 10⁻¹⁰ to 10⁻¹² moles/litre or less and more preferably 5×10⁻¹⁰ to 10⁻¹¹ moles/litre, 5×10⁻¹⁰ to 5×10⁻¹¹ moles/litre, 10⁻¹⁰ to 10⁻¹² moles/litre, or 10⁻¹⁰ to 10⁻¹¹ moles/litre, such as 4.3×10⁻¹¹ moles/litre, as determined by Surface Plasmon Resonance.
66. The ISVD according to any one of items 1 to 65, wherein the ISVD specifically binds to human PD-1 with a kon-rate of between 10⁵ M⁻¹ s⁻¹ to about 10⁷ M⁻¹ s⁻¹, preferably between 5×10⁵ M⁻¹ s⁻¹ and 10⁷ M⁻¹ s⁻¹, more preferably between 10⁵ M⁻¹ s⁻¹ and 5×10⁶ M⁻¹ s⁻¹, such as between 10⁵ M⁻¹ s⁻¹ and 1×10⁶ M⁻¹ s⁻¹, such as 3×10⁵ M⁻¹ s⁻¹, as determined by Surface Plasmon Resonance.
67. The ISVD according to any one of items 1 to 66, wherein the ISVD specifically binds to human PD-1 with a k_{off} rate between 10⁻³ s⁻¹ (t1/2=0.69 s) and 108 s⁻¹ (providing a near irreversible complex with a t1/2 of multiple days), preferably 10⁻³ s⁻¹ and 5×10⁻⁶ s⁻¹, more preferably between 5×10⁻⁴ s″ 1 and 5×10⁻⁶ s⁻¹, such as between 5×10⁻⁴ s⁻¹ and 105 s⁻¹, such as 1.3×10⁻⁵ s⁻¹, determined by Surface Plasmon Resonance.
68. The ISVD according to any one of items 1 to 67, wherein the ISVD specifically binds to human and cyno PD-1 and does not bind to CD28, CTLA-4, ICOS, and BTLA or other members of the CD28 receptor family.
69. The ISVD according to any one of items 1 to 68, wherein the ISVD antagonizes an activity of PD-L1.
70. The ISVD according to any one of items 1 to 69, wherein the ISVD blocks the interaction of human PD-1 with human PD-L1 with a potency (IC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 10⁻⁹ M, between 10⁻¹⁰ M and 10⁻⁸ M or between 10⁻⁹ M and 10⁻⁸ M, such as 5.11×10⁻⁹ M, for example, as measured in a FACS competition assay on CHO cells expressing human PD-1 with hPD-L1 as a competitor.
71. The ISVD according to any one of items 1 to 70, wherein the ISVD blocks the interaction of cyno PD-1 with cyno PD-L1 with a potency (IC50 value) of 10⁷ M or lower, of 5×10⁻⁸ M or lower, more preferably of 10⁻⁸ M or lower, or even of 5×10⁻⁹ M or lower, such as between 10⁻¹¹ M and 10⁻⁷ M, between 10⁻¹¹ M and 10⁻⁸ M, between 10⁻¹⁰ M and 10⁻⁷ M, between 10⁻¹⁰ M and 10⁻⁹ M, between 10⁻¹⁰ M and 10⁻⁸ M, between 10⁻⁹ M and 10⁻⁸ M or between 10⁻⁹ M and 10⁷ M, such as 5.76×10⁻⁸ M, for example, as measured in a FACS competition assay on CHO cells expressing cyno PD-1 with hPD-L1 as a competitor.
72. The ISVD according to any one of items 1 to 71, wherein the ISVD blocks the binding of hPD-1 to hPD-L1 by at least 50%, such as at least 60%, 70%, 80%, 90%, 95%, 99% or even more, as determined by ligand competition, AlphaScreen, or competitive binding assays (such as competition ELISA or competition FACS).
73. The ISVD according to any one of items 1 to 72, wherein the ISVD induces T-cell activation with a potency (EC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 10⁻⁸ M, between 10⁻¹⁰ M and 10⁻⁹ M or between 10⁻⁹ M and 10⁻⁸ M, such as 2.2×10⁹ M or 2.8×10⁹ M, as determined in a TT assay.
74. A polypeptide or construct that comprises or essentially consists of one or more ISVDs according to any one of items 1 to 73, and optionally further comprises one or more other groups, residues, moieties or binding units, optionally linked via one or more linkers.
75. The polypeptide or construct according to item 74, in which said one or more other groups, residues, moieties or binding units are amino acid sequences.
76. The polypeptide or construct according to any one of items 74 to 75, in which said one or more linkers are one or more amino acid sequences.
77. The polypeptide or construct according to any one of items 74 to 76, in which said one or more other groups, residues, moieties or binding units are immunoglobulin sequences.
78. The polypeptide or construct according to any one of items 74 to 77, in which said one or more other groups, residues, moieties or binding units are ISVDs.
79. The polypeptide or construct according to any one of items 74 to 78, in which said one or more other groups, residues, moieties or binding units are chosen from the group consisting of VHHs, humanized VHHs, camelized VHs, domain antibodies, single domain antibodies and dAbs.
80. The polypeptide or construct according to any one of items 74 to 79, which is a multivalent construct.
81. The polypeptide or construct according to any one of items 74 to 80, which is a multispecific construct.
82. The polypeptide or construct according to any one of items 74 to 81, in which said one or more other groups, residues, moieties or binding units provide the polypeptide or construct with increased half-life, compared to the ISVD without the one or more other groups, residues, moieties or binding units.
83. The polypeptide or construct according to item 82, in which said one or more other groups, residues, moieties or binding units that provide the polypeptide or construct with increased half-life is chosen from the group consisting of a polyethylene glycol molecule (PEG), serum proteins or fragments thereof, binding units that specifically bind to serum proteins, an Fc portion, and small proteins or peptides that specifically bind to serum proteins.
84. The polypeptide or construct according to item 83, in which said one or more other groups, residues, moieties or binding units that provide the polypeptide or construct with increased half-life is chosen from the group consisting of human serum albumin or fragments thereof.
85. The polypeptide or construct according to item 83, in which said one or more other groups, residues, moieties or binding units that provides the polypeptide or construct with increased half-life are chosen from the group consisting of binding units that specifically bind to serum albumin (such as human serum albumin) or a serum immunoglobulin (such as IgG).
86. The polypeptide or construct according to item 85, in which said one or more other groups, residues, moieties or binding units that provides the polypeptide or construct with increased half-life are chosen from the group consisting of VHHs, humanized VHHs, camelized VHs, domain antibodies, single domain antibodies, or dAbs that specifically bind to serum albumin (such as human serum albumin) or a serum immunoglobulin (such as IgG).
87. The polypeptide or construct according to item 85 or 86, in which said one or more other groups, residues, moieties or binding units that provides the polypeptide or construct with increased half-life is an ISVD that specifically binds human serum albumin.
88. The polypeptide or construct according to item 87, wherein said ISVD that specifically binds human serum albumin essentially consists of 4 framework regions (FR1 to FR4, respectively) and 3 complementarity determining regions (CDR1 to CDR3 respectively), in which:
   CDR1 (AbM numbering) has an amino acid sequence selected from:
      a) the amino acid sequence of SEQ ID NO: 2436;
      b) amino acid sequences that have at least 80% amino acid identity with the amino acid sequence of SEQ ID NO: 2436; and
      c) amino acid sequences that have 3, 2, or 1 amino acid difference with the amino acid sequence of SEQ ID NO: 2436;
and
   CDR2 (AbM numbering) has an amino acid sequence selected from:
      a) the amino acid sequence of SEQ ID NO: 2438;
      b) amino acid sequences that have at least 80% amino acid identity with the amino acid sequence of SEQ ID NO: 2438;
      c) amino acid sequences that have 3, 2, or 1 amino acid difference with the amino acid sequence of SEQ ID NO: 2438;
and
   CDR3 (AbM numbering) has an amino acid sequence selected from:
      a) the amino acid sequence of SEQ ID NO: 2440;
      b) amino acid sequences that have at least 80% amino acid identity with the amino acid sequence of SEQ ID NO: 2440;
      c) amino acid sequences that have 3, 2, or 1 amino acid difference with the amino acid sequence of SEQ ID NO: 2440.
89. The polypeptide or construct according to item 88, wherein CDR1 consists of the amino acid sequence of SEQ ID NO: 2436, CDR2 consists of the amino acid sequence of SEQ ID NO: 2438, and CDR3 consists of the amino acid sequence of SEQ ID NO: 2440.
90. The polypeptide or construct according to any one of items 87 to 89, wherein said ISVD that specifically binds human serum albumin is selected from the group consisting of ALB8 (SEQ ID NO: 2401), ALB23 (SEQ ID NO: 2402), ALBX00001 (SEQ ID NO: 2415) and ALB23002 (SEQ ID NO: 2416), preferably the ISVD binding to human serum albumin is ALB23002 (SEQ ID NO: 2416).
91. The polypeptide or construct according to any one of items 74 to 90, wherein said linker is chosen from the group consisting of SEQ ID NOs: 2418 to 2434.
92. The polypeptide or construct according to any one of items 74 to 91, further comprising a C-terminal extension.
93. The polypeptide or construct according to item 92, wherein said C-terminal extension is a C-terminal extension (X)ₙ (SEQ ID NO: 4796), in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I).
94. A nucleic acid that encodes an ISVD according to any one of items 1 to 73, or a polypeptide according to any one of items 74 to 93.
95. The nucleic acid according to item 94, that is in the form of a genetic construct.
96. A non-human host or host cell that expresses, or that under suitable circumstances is capable of expressing, an ISVD according to any one of items 1 to 73, or a polypeptide according to any one of items 74 to 93; and/or that comprises the nucleic acid according to any one of items 94 or 95.
97. A method for producing an ISVD according to any one of items 1 to 73, or a polypeptide according to any one of items 74 to 93, at least comprising the steps of:
   a) expressing, in a suitable non-human host cell or host organism or in another suitable expression system, a nucleic acid according to any one of items 94 or 95;
   optionally followed by:
   b) isolating and/or purifying the ISVD according to any one of items 1 to 73, or the polypeptide according to any one of items 74 to 93.
98. A method for producing an ISVD according to any one of items 1 to 73, or a polypeptide according to any one of items 74 to 93, said method at least comprising the steps of:
   a) cultivating and/or maintaining a non-human host or host cell according to item 96 under conditions that are such that said non-human host or host cell expresses and/or produces at least one ISVD according to any one of items 1 to 73, or at least one polypeptide according to any one of items 74 to 93;
   optionally followed by:
   b) isolating and/or purifying the ISVD according to any one of items 1 to 73, or polypeptide according to any one of items 74 to 93.
99. A composition comprising at least one ISVD according to any one of items 1 to 73, at least one polypeptide or construct according to any one of items 74 to 93, or at least one nucleic acid according to any one of items 94 or 95.
100. The composition according to item 99, which is a pharmaceutical composition.
101. The composition according to any one of items 99 or 100, which is a pharmaceutical composition, that further comprises at least one pharmaceutically acceptable carrier, diluent or excipient and/or adjuvant, and that optionally comprises one or more further pharmaceutically active polypeptides and/or compounds.
102. The ISVD according to any one of items 1 to 73, the polypeptide or construct according to any one of items 74 to 93, or the composition according to any one of items 99 to 101, for use as a medicament.
103. The ISVD according to any one of items 1 to 73, the polypeptide or construct according to any one of items 74 to 93, or the composition according to any one of items 99 to 101, for use in the diagnosis, prevention and/or treatment of at least one disease and/or disorder.
104. The ISVD according to any one of items 1 to 73, the polypeptide or construct according to any one of items 74 to 93, or the composition according to any one of items 99 to 101, for use in the diagnosis, prevention and/or treatment of at least one disease and/or disorder that is associated with PD-1, with its biological or pharmacological activity, and/or with the biological pathways or signalling in which PD-1 is involved.
105. The ISVD according to any one of items 1 to 73, the polypeptide or construct according to any one of items 74 to 93, or the composition according to any one of items 99 to 101, for use in the diagnosis, prevention and/or treatment of cancer.
106. A method for the diagnosis, prevention and/or treatment of at least one disease and/or disorder, comprising the administration, to a subject in need thereof, of a pharmaceutically active amount of an ISVD according to any one of items 1 to 73, a polypeptide or construct according to any one of items 74 to 93, or a composition according to any one of items 99 to 101.
107. The method according to item 106, for the diagnosis, prevention and/or treatment of at least one disease or disorder that is associated with PD-1, with its biological or pharmacological activity, and/or with the biological pathways or signalling in which PD-1 is involved, said method comprising administering, to a subject in need thereof, a pharmaceutically active amount of at least one ISVD according to any one of items 1 to 73, a polypeptide or construct according to any one of items 74 to 93, or composition according to any one of items 99 to 101.
108. The method according to any one of items 106 or 107, for the diagnosis, prevention and/or treatment of cancer, said method comprising administering, to a subject in need thereof, a pharmaceutically active amount of at least one ISVD according to any one of items 1 to 73, a polypeptide or construct according to any one of items 74 to 93, or a composition according to any one of items 99 to 101.
7.2 Embodiment 2, Family A0294041A11
1. An immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1 protein, wherein at least one amino acid residue located in at least one of the CDRs is capable of interacting with at least one amino acid residue in at least one, preferably in each, of the following stretches (of the ectodomain) of human PD-1 protein, when numbered in accordance with SEQ ID NO: 4703: N66-E84, and I126-E136, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.
2. The ISVD according to item 1, wherein the at least one amino acid residue in at least one, preferably in each stretch (of the ectodomain) of human PD-1 protein is selected from:
   For stretch N66-E84: N66, Y68, N74, Q75, T76, D77, K78, P83, and E84;
   For stretch I126-E136: I126, L128, A129, P130, K131, A132, Q133, I134, and E136, when numbered in accordance with SEQ ID NO: 4703.
3. The ISVD according to any one of the preceding items, wherein at least one amino acid residue located in at least one of the CDRs is capable of interacting with at least two, at least three, at least four, or all of the following amino acid residues in stretch N66-E84 (of the ectodomain) of human PD-1 protein, when numbered in accordance with SEQ ID NO: 4703: N66, Y68, N74, Q75, T76, D77, K78, P83, and E84.
4. The ISVD according to any one of the preceding items, wherein at least one amino acid residue located in at least one of the CDRs is capable of interacting with at least two, at least three, at least four, at least five, at least six, at least seven, at least eight, or all of the following amino acid residues in stretch I126-E136 (of the ectodomain) of human PD-1 protein when numbered in accordance with SEQ ID NO: 4703: I126, L128, A129, P130, K131, A132, Q133, I134, and E136.
5. The ISVD according to any one of the preceding items, wherein at least one amino acid residue located in at least one of the CDRs is capable of forming salt bridges with at least the amino acid residue K78 of human PD-1 protein, when numbered in accordance with SEQ ID NO: 4703, optionally wherein at least one amino acid residue located in at least one of the CDRs is further capable of forming salt bridges with the amino acid residue E136 of human PD-1 protein, when numbered in accordance with SEQ ID NO: 4703, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.
6. The ISVD according to any one of the preceding items, wherein at least one amino acid residue located in at least one of the CDRs is capable of forming hydrogen bonds with at least one, preferably all, of the following amino acid residues of human PD-1 protein, when numbered in accordance with SEQ ID NO: 4703: N66, A129, P130, and K131, optionally wherein at least one amino acid residue located in at least one of the CDRs is further capable of forming hydrogen bonds with the amino acid residue N74 of human PD-1 protein, when numbered in accordance with SEQ ID NO: 4703, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.
7. The ISVD according to any one of the preceding items, wherein the least one amino acid residue located in at least one of the CDRs is capable of interacting with an (discontinuous) epitope on human PD-1 comprising at least one amino acid residue in each of the following stretches (of the ectodomain) of human PD-1 protein: N66-E84, and I126-E136, when numbered in accordance with SEQ ID NO: 4703.
8. The ISVD according to item 7, wherein the at least one amino acid residue in each stretch (of the ectodomain) of human PD-1 protein is selected from:
   For stretch N66-E84: N66, Y68, N74, Q75, T76, D77, K78, P83, and E84;
   For stretch I126-E136: I126, L128, A129, P130, K131, A132, Q133, I134, and E136, when numbered in accordance with SEQ ID NO: 4703.
9. The ISVD according to any one of the preceding items, wherein at least one amino acid residue located in at least one of the CDRs is capable of interacting with at least one, at least two, at least three, at least four, at least five, at least six, at least seven, at least ten, at least fifteen, preferably all, of the following amino acid residues of human PD-1 protein, when numbered in accordance with SEQ ID NO: 4703: N66, Y68, N74, Q75, T76, D77, K78, P83, E84, I126, L128, A129, P130, K131, A132, Q133, I134, and E136, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.
10. The ISVD according to any one of the preceding items, wherein the amino acid residue located in at least one of the CDRs which are capable of interacting with amino acid residues of human PD-1 protein are located in CDR1 and/or CDR3.
11. The ISVD according to any one of the preceding items, wherein the:
   amino acid at position 101 (Kabat numbering) is capable of interacting, preferably by forming a salt bridge, with K78 in PD-1; and/or
   amino acid at position 99 (Kabat numbering) is capable of interacting, preferably by forming a salt bridge, with E136 in PD-1;
preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.
12. The ISVD according to any one of the preceding items, wherein:
   the amino acid residue at position 29 (Kabat numbering) is capable of interacting, preferably by forming a hydrogen bond, with A129 in PD-1;
   the amino acid residue at position 30 (Kabat numbering) is capable of interacting, preferably by forming a hydrogen bond, with P130 in PD-1;
   the amino acid residue at position 31 (Kabat numbering) is capable of interacting, preferably by forming a hydrogen bond, with P130 in PD-1;
   the amino acid residue at position 32 (Kabat numbering) is capable of interacting, preferably by forming a hydrogen bond, with P130 in PD-1;
   the amino acid residue at position 31 (Kabat numbering) is capable of interacting, preferably by forming a hydrogen bond, with K131 in PD-1;
   the amino acid residue at position 101 (Kabat numbering) is capable of interacting, preferably by forming a hydrogen bond, with N66 in PD-1; and/or.
   the amino acid residue at position 99 (Kabat numbering) is capable of interacting, preferably by forming a hydrogen bond, with N74 in PD-1;
preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.
13. The ISVD according to any one of the preceding items, wherein two amino acids are considered to interact when the distance between them is 4.0 Å or less.
14. The ISVD according to any one of the preceding items, wherein the interacting amino acid residue(s) in the ISVD and in human PD-1 protein are identified by X-ray crystallography by:
   Mixing human PD-1 protein, and the ISVD in PBS1× buffer;
   Concentrating the protein complex to 8 mg/ml;
   Crystallizing by hanging drop vapor diffusion using 100 mM Na Cacodylate, 300 mM Na Acetate, 10% PEG1000, pH 6.5 at 19° C.;
   Collecting X-ray diffraction data;
   Processing the collected data,
preferably as described in Example 13.
15. The ISVD according to any one of the preceding items, wherein the (discontinuous) epitope comprises or consists of the following amino acid residues of human PD-1 protein, when numbered in accordance with SEQ ID NO: 4703: N66, Y68, N74, Q75, T76, D77, K78, P83, E84, I126, L128, A129, P130, K131, A132, Q133, I134, and E136.
16. The ISVD according to any one of the preceding items, wherein the ISVD cross-blocks the binding to human PD-1 protein of a polypeptide having an amino acid sequence selected from SEQ ID NOs: 714-1871, preferably wherein the ISVD cross-blocks the binding to human PD-1 protein of a polypeptide having an amino acid sequence selected from SEQ ID NO: 1486, or wherein the binding of the ISVD to human PD-1 protein is cross-blocked by a polypeptide having an amino acid sequence selected from SEQ ID NOs: 714-1871.
17. An immunoglobulin single variable domain (ISVD) specifically binding human PD-1 protein, that essentially consists of 4 framework regions (FR1 to FR4, respectively) and 3 complementarity determining regions (CDR1 to CDR3, respectively), wherein the ISVD comprises an amino acid residue capable of forming salt bridges at at least position 101 (Kabat numbering), and optionally further at position 99 (Kabat numbering), preferably wherein the ISVD comprises at least the following amino acid residue at the following positions: D101, and optionally K99 (Kabat numbering).
18. The ISVD according to any one of the preceding items, wherein the ISVD comprises an amino acid residue capable of forming hydrogen bonds at at least one, preferably at all, of the following positions: 29, 30, 31, 32, 101 (Kabat numbering) and optionally further at position 99 (Kabat numbering), preferably wherein the ISVD comprises at least one, preferably all of the following amino acid residues at the following positions: F29, S30, E31, N32, and D101 and optionally K99 (Kabat numbering).
19. The ISVD according to any one of the preceding items, wherein the ISVD comprises at least one, at least two, at least three, preferably all of the following amino acid residues at the following positions (Kabat numbering): D27, T28, F29, S30, E31, and N32, preferably at least F29, S30, E31, and N32.
20. The ISVD according to any one of the preceding items, wherein the ISVD comprises at least one, at least two, at least three, at least four, preferably all of the following amino acid residues at the following positions (Kabat numbering): 196, F97, K99, W100g, and D101, preferably at least K99 and D101.
21. The ISVD according to any one of the preceding items, wherein:
   the CDR1 (AbM definition) consists of the following amino acid sequence X₁DTFSENX₂X₃X₄ (SEQ ID NO: 4694); wherein
      X₁ is selected from any amino acid, preferably X₁ is selected from G, K, F, A, Y, T, V, H, R, S, P, I, E, L, or D;
      X₂ is selected from any amino acid, preferably X₂ is selected from D, P, A, T, I, R, V, G, Y, L, F, K, S, I, H, or E;
      X₃ is selected from any amino acid, preferably X₃ is selected from I, R, H, M, G, Y, Q, E, K, F, T, S, A, D, or P;
      X₄ is selected from any amino acid, preferably X₄ is selected from G, A, L, S, R, H, V, P, Q, E, I, D, Y, T, or K; and/or
   the CDR3 (AbM definition) consists of the following amino acid sequence X₁IF X₂KX₃X₄X₅X₆X₇X₈X₉WX₁₀X₁₁DX₁₂ (SEQ ID NO: 4695); wherein:
      X₁ is selected from any amino acid, preferably X₁ is selected from K, R, L, Y, T, I, Q, A, F, P, D, H, V, S, or E;
      X₂ is selected from any amino acid, preferably X₂ is selected from S, D, G, F, L, K, I, P, Y, V, A, or T;
      X₃ is selected from any amino acid, preferably X₃ is selected from T, K, A, F, S, G, D, P, H, I, Q, Y, L, or V;
      X₄ is selected from any amino acid, preferably X₄ is selected from G, F, H, Y, I, L, T, E, P, R, S, Q, D, K, or A;
      X₅ is selected from any amino acid, preferably X₅ is selected from T, E, G, V, S, Q, L, H, R, P, A, F, K, I, or Y;
      X₆ is selected from any amino acid, preferably X₆ is selected from Y, I, A, Q, G, E, F, D, K, L, S, T, R, or V;
      X₇ is selected from any amino acid, preferably X₇ is selected from S, T, G, L, Q, A, I, K, E, H, D, V, R, P, F, or Y;
      X₈ is selected from any amino acid, preferably X₈ is selected from A, H, Y, V, R, F, G, L, D, E, T, I, K, P, or S;
      X₉ is selected from any amino acid, preferably X₉ is selected from S, T, I, D, H, V, R, P, L, K, G, Y, V, E, F, or A;
      X₁₀ is selected from any amino acid, preferably X₁₀ is selected from E, R, P, I, T, A, F, D, S, H, V, K, L, G, Y, or Q;
      X₁₁ is selected from any amino acid, preferably X₁₁ is selected from Y, Q, L, G, V, P, K, E, I, R, H, D, or F; and
      X₁₂ is selected from any amino acid, preferably X₁₂ is selected from F, S, T, Y, K, R, A, H, G, I, V, D, Q, P, E, or L.
22. The ISVD according to any one of the preceding items, wherein the following amino acid residue of the ISVD at the following position: D101 (Kabat numbering) and optionally the following amino acid residue of the ISVD at the following position: K99 (Kabat numbering) are capable of forming salt bridges with at least one of the amino acid residues of human PD-1 protein, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.
23. The ISVD according to any one of the preceding items, wherein at least one, preferably all of the following amino acid residues of the ISVD at the following positions: F29, S30, E31, N32, and D101 (Kabat numbering) and optionally K99 (Kabat numbering) are capable of forming hydrogen bonds with at least one of the amino acid residues of human PD-1 protein, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.
24. The ISVD according to any one of the preceding items, wherein at least one, preferably all, of the following amino acid residues of the ISVD at the following positions: F29, S30, E31, N32, and D101 and optionally K99 (Kabat numbering) is capable of interacting with at least one, preferably all of the following amino acid residues of human PD-1 protein, when numbered in accordance with SEQ ID NO: 4703: N66, K78, A129, P130, and K131, and optionally N74, preferably wherein:
   F29 in the ISVD (Kabat numbering) interacts, preferably by forming a hydrogen bond, with A129 in human PD-1 protein;
   S30 in the ISVD (Kabat numbering) interacts, preferably by forming a hydrogen bond, with P130 in human PD-1 protein;
   E31 in the ISVD (Kabat numbering) interacts, preferably by forming a hydrogen bond, with P130 in human PD-1 protein;
   E31 in the ISVD (Kabat numbering) interacts, preferably by forming a hydrogen bond, with K131 in human PD-1 protein;
   N32 in the ISVD (Kabat numbering) interacts, preferably by forming a hydrogen bond, with P130 in human PD-1 protein,
   K99 in the ISVD (Kabat numbering) interacts, preferably by forming a hydrogen bond, with N74 in human PD-1 protein,
   D101 in the ISVD (Kabat numbering) interacts, preferably by forming a hydrogen bond, with N66 in human PD-1 protein,
   D101 in the ISVD (Kabat numbering) interacts, preferably by forming a salt bridge, with K78 in human PD-1 protein,
   K99 in the ISVD (Kabat numbering) interacts, preferably by forming a salt bridge, with E136 in human PD-1 protein,
preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.
25. The ISVD according to any one of the preceding items, wherein the ISVD comprises at least one, at least two, at least three, at least four, at least five, preferably all of the following amino acid residues at the following positions (Kabat numbering): F29, S30, E31, N32, K99, and D101.
26. The ISVD according to any one of the preceding items, wherein the ISVD comprises at least one, at least two, at least three, at least four, at least five, at least six, at least seven, at least eight, at least nine, preferably all of the following amino acid residues at the following positions (Kabat numbering): D27, T28, F29, S30, E31, N32, 196, F97, K99, W100g, and D101.
27. The ISVD according to any one of the preceding items, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of SEQ ID NO: 1957;
      b) amino acid sequences that have at least 80% amino acid identity with the amino acid sequence of SEQ ID NO: 1957; and
      c) amino acid sequences that have 3, 2, or 1 amino acid difference with the amino acid sequences of SEQ ID NO: 1957;
and
   CDR3 (AbM definition) has an amino acid sequence selected from:
      a) the amino acid sequence of SEQ ID NOs: 2337;
      b) amino acid sequences that have at least 80% amino acid identity with the amino acid sequence of SEQ ID NO: 2337;
      c) amino acid sequences that have 3, 2, or 1 amino acid difference with the amino acid sequence of SEQ ID NO: 2337.
28. The ISVD according to item 27, wherein the ISVD comprises D1 and/or V2 (Kabat numbering).
29. The ISVD according to any one of the preceding items, wherein the ISVD comprises at least one, at least two, at least three, at least four, at least five, at least six, at least seven, at least eight, at least nine, at least ten, at least eleven, at least twelve, preferably all of the following amino acid residues at the following positions (Kabat numbering): D1, V2, D27, T28, F29, S30, E31, N32, 196, F97, K99, W100g, and D101.
30. The ISVD according to any one of the preceding items, in which F29, S30, E31, N32, and D101 and optionally K99 (Kabat numbering) of the ISVD are capable of interacting with at least one of the following amino acids of human PD-1 protein, when numbered in accordance with SEQ ID NO: 4703: N66, Y68, N74, Q75, T76, D77, K78, P83, E84, I126, L128, A129, P130, K131, A132, Q133, 1134, and E136, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.
31. The ISVD according to any one of the preceding items, in which D1, V2, D27, T28, F29, S30, E31, N32, 196, F97, K99, W100g, and D101 (Kabat numbering) of the ISVD are capable of interacting with the following amino acids of human PD-1 protein, when numbered in accordance with SEQ ID NO: 4703: N66, Y68, N74, Q75, T76, D77, K78, P83, E84, I126, L128, A129, P130, K131, A132, Q133, I134, and E136, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.
32. The ISVD according to any one of the preceding items, wherein two amino acids are considered to interact when the distance between them is 4.0 Å or less.
33. The ISVD according to any one of the preceding items, wherein the interacting amino acid residue(s) in the ISVD and in human PD-1 protein are identified by X-ray crystallography by:
   Mixing human PD-1 protein, and the ISVD in PBS1× buffer;
   Concentrating the protein complex to 8 mg/ml;
   Crystallizing by hanging drop vapor diffusion using 100 mM Na Cacodylate, 300 mM Na Acetate, 10% PEG1000, pH 6.5 at 19° C.;
   Collecting X-ray diffraction data;
   Processing the collected data,
preferably as described in Example 13.
34. An immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1, wherein the ISVD cross-blocks the binding to human PD-1 protein of a polypeptide with the amino acid sequence of SEQ ID Nos: 714-1871 or wherein the ISVD is cross-blocked from binding to human PD-1 protein by a polypeptide with the amino acid sequence of SEQ ID Nos: 714-1871.
35. The ISVD according to any one of the preceding items, wherein the ISVD competes with a polypeptide with the amino acid sequence of SEQ ID No: 714-1871 for binding human PD-1 protein, as measured in a competition assay, such as a FACS competition assay.
36. An immunoglobulin single variable domain (ISVD) specifically binding human PD-1, that essentially consists of 4 framework regions (FR1 to FR4, respectively) and 3 complementarity determining regions (CDR1 to CDR3, respectively), in which:
   CDR1 (AbM definition) has an amino acid sequence selected from:
      a) the amino acid sequence of X₁X₂X₃X₄X₅X₆X₇X₈X₉X₁₀ (SEQ ID NO: 4691); wherein X₁ is selected from G, K, F, A, Y, T, V, H, R, S, P, I, E, L, or D, X₂ is selected from D, Y, R, E, T, L, K, H, V, S, P, Q, I, G, A, or F, X₃ is selected from T, I, A, E, H, G, P, F, L, K, Q, V, R, Y, or S, X₄ is selected from F, K, D, L, T, I, A, E, V, G, H, Y, Q, S, or R, X₅ is selected from S, I, E, A, F, R, V, Y, L, T, Q, K, D, G, or H, X₆ is selected from E, Q, V, T, S, F, K, P, I, G, R, L, H, or A, X₇ is selected from N, R, Q, V, E, Y, H, A, K, L, P, G, F, I, S, or D, X₈ is selected from D, P, A, T, I, R, V, G, Y, L, F, K, S, I, H, or E, X₉ is selected from I, R, H, M, G, Y, Q, E, K, F, T, S, A, D, or P, and X₁₀ is selected from G, A, L, S, R, H, V, P, Q, E, I, D, Y, T or K;
      b) amino acid sequences that have at least 80% amino acid identity with the amino acid sequence of X₁X₂X₃X₄X₅X₆X₇X₈X₉X₁₀ (SEQ ID NO: 4691); wherein X₁ is selected from G, K, F, A, Y, T, V, H, R, S, P, I, E, L, or D, X₂ is selected from D, Y, R, E, T, L, K, H, V, S, P, Q, I, G, A, or F, X₃ is selected from T, I, A, E, H, G, P, F, L, K, Q, V, R, Y, or S, X₄ is selected from F, K, D, L, T, I, A, E, V, G, H, Y, Q, S, or R, X₅ is selected from S, I, E, A, F, R, V, Y, L, T, Q, K, D, G, or H, X₆ is selected from E, Q, V, T, S, F, K, P, I, G, R, L, H, or A, X₇ is selected from N, R, Q, V, E, Y, H, A, K, L, P, G, F, I, S, or D, X₈ is selected from D, P, A, T, I, R, V, G, Y, L, S, I, H, or E, X₉ is selected from I, R, H, M, G, Y, Q, E, K, F, T, S, A, D, or P, and X₁₀ is selected from G, A, L, S, R, H, V, P, Q, E, I, D, Y, T, or K;
      c) amino acid sequences that have 3, 2, or 1 amino acid difference with the amino acid sequences of X₁X₂X₃X₄X₅X₆X₇X₈X₉X₁₀ (SEQ ID NO: 4691); wherein X₁ is selected from G, K, F, A, Y, T, V, H, R, S, P, I, E, L, or D, X₂ is selected from D, Y, R, E, T, L, K, H, V, S, P, Q, I, G, A, or F, X₃ is selected from T, I, A, E, H, G, P, F, L, K, Q, V, R, Y, or S, X₄ is selected from F, K, D, L, T, I, A, E, V, G, H, Y, Q, S, or R, X₅ is selected from S, I, E, A, F, R, V, Y, L, T, Q, K, D, G, or H, X₆ is selected from E, Q, V, T, S, F, K, P, I, G, R, L, H, or A, X₇ is selected from N, R, Q, V, E, Y, H, A, K, L, P, G, F, I, S, or D, X₈ is selected from D, P, A, T, I, R, V, G, Y, L, S, I, H, or E, X₉ is selected from I, R, H, M, G, Y, Q, E, K, F, T, S, A, D, or P, and X₁₀ is selected from G, A, L, S, R, H, V, P, Q, E, I, D, Y, T, or K;
and
   CDR2 (AbM definition) has an amino acid sequence selected from:
      a) the amino acid sequence of X₁X₂X₃X₄X₅X₆X₇X₈X₉X₁₀ (SEQ ID NO: 4692); wherein X₁ is selected from A, G, P, F, V, I, R, Q, H, T, S, Y, K, or L, X₂ is selected from I, H, K, G, L, P, F, A, V, T, E, Y, S, Q, D, or R, X₃ is selected from V, K, Y, F, G, Q, A, T, S, P, I, R, D, or L, X₄ is selected from W, P, Y, Q, G, T, D, H, R, K, S, L, I, V, F, or A, X₅ is selected from P, K, Y, F, G, I, L, T, Q, H, E, D, A, S, or V, X₆ is selected from G, Q, E, P, L, F, K, I, S, A, D, R, H, or V, X₇ is selected from G, A, H, F, L, Y, T, P, Q, K, I, R, D, V, or S, X₈ is selected from E, T, I, R, H, Q, D, K, S, P, A, F, Y, V, G, or L, X, is selected from T, E, L, Y, F, P, Q, I, R, S, D, K, A, V, or H, and X₁₀ is selected from Y, G, I, L, E, D, H, K, A, F, S, R, P, T, Q;
      b) amino acid sequences that have at least 80% amino acid identity with the amino acid sequence of X₁X₂X₃X₄X₅X₆X₇X₈X₉X₁₀ (SEQ ID NO: 4692); wherein X₁ is selected from A, G, P, F, V, I, R, Q, H, A, T, S, Y, K, or L, X₂ is selected from I, H, K, G, L, P, F, A, V, T, E, Y, S, Q, D, or R, X₃ is selected from V, K, Y, F, G, Q, A, T, S, P, I, R, D, or L, X₄ is selected from W, P, Y, Q, G, T, D, H, R, K, S, L, I, V, F, or A, X₅ is selected from P, K, Y, F, G, I, L, T, Q, H, E, D, A, S, or V, X₆ is selected from G, Q, E, P, L, F, K, I, S, A, D, R, H, or V, X₇ is selected from G, A, H, F, L, Y, T, P, Q, K, I, R, D, V, or S, X₈ is selected from E, T, I, R, H, Q, D, K, S, P, A, F, Y, V, G, L, or E, X₉ is selected from T, E, L, Y, F, P, Q, I, S, D, K, A, V, or H, and X₁₀ is selected from Y, G, I, L, E, D, H, K, A, F, S, R, P, T, Q;
      c) amino acid sequences that have 3, 2, or 1 amino acid difference with the amino acid sequence of X₁X₂X₃X₄X₅X₆X₇X₈X₉X₁₀ (SEQ ID NO: 4692); wherein X₁ is selected from A, G, P, F, V, I, R, Q, H, A, T, S, Y, K, or L, X₂ is selected from I, H, K, G, L, P, F, A, V, T, E, Y, S, Q, D, or R, X₃ is selected from V, K, Y, F, G, Q, A, T, S, P, I, R, D, or L, X₄ is selected from W, P, Y, Q, G, T, D, H, R, K, S, L, I, V, F, or A, X₅ is selected from P, K, Y, F, G, I, L, T, Q, H, E, D, A, S, or V, X₆ is selected from G, Q, E, P, L, F, K, I, S, A, D, R, H, or V, X₇ is selected from G, A, H, F, L, Y, T, P, Q, K, I, R, D, V, or S, X₈ is selected from E, T, I, R, H, Q, D, K, S, P, A, F, Y, V, G, L, or E, X₉ is selected from T, E, L, Y, F, P, Q, I, S, D, K, A, V, or H, and X₁₀ is selected from Y, G, I, L, E, D, H, K, A, F, S, R, P, T, Q;
and
   CDR3 (AbM definition) has an amino acid sequence selected from:
      a) the amino acid sequence of X₁X₂X₃X₄X₅X₆X₇X₈X₉X₁₀X₁₁X₁₂X₁₃X₁₄X₁₅X₁₆X₁₇ (SEQ ID NO: 4693); wherein X₁ is selected from K, R, L, Y, T, I, Q, A, F, P, D, H, V, S, or E, X₂ is selected from I, H, T, E, Q, S, L, K, D, G, Y, V, F, R, A, or P, X₃ is selected from F, E, K, A, R, Q, H, T, D, V, L, G, I, Y, or S, X₄ is selected from S, D, G, F, L, K, I, P, Y, V, A, or T, X₅ is selected from K, T, R, P, Y, S, E, I, L, H, D, F, V, A, or G, X₆ is selected from T, K, A, F, S, G, D, P, H, I, Q, Y, L, or V, X₇ is selected from G, F, H, Y, I, L, T, E, P, R, S, Q, D, K, or A, X₈ is selected from T, E, G, V, S, Q, L, H, R, P, A, F, K, I, or Y, X, is selected from Y, I, A, Q, G, E, F, D, K, L, S, T, R or V, X₁₀ is selected from S, T, G, L, Q, A, I, K, E, H, D, V, R, P, F, or Y, X₁₁ is selected from A, H, Y, V, R, F, G, L, D, E, T, I, K, P, or S, X₁₂ is selected from S, T, I, D, H, V, R, P, L, K, G, Y, V, E, F, or A, X₁₃ is selected from W, Q, F, H, D, S, K, I, T, E, G, R, L, Y, or A, X₁₄ is selected from E, R, P, I, T, A, F, D, S, H, V, K, L, G, Y, or Q, X₁₅ is selected from Y, Q, L, G, V, P, K, E, I, R, H, D, or F, X₁₆ is selected from D, L, E, F, P, R, T, I, S, Y, G, A, V, K, or H, and X₁₇ is selected from F, S, T, Y, K, R, A, H, G, I, V, D, Q, P, E, or L;
      b) amino acid sequences that have at least 80% amino acid identity with the amino acid sequence of X₁X₂X₃X₄X₅X₆X₇X₈X₉X₁₀X₁₁X₁₂X₁₃X₁₄X₁₅X₁₆X₁₇ (SEQ ID NO: 4693); wherein X₁ is selected from K, R, L, Y, T, I, Q, A, F, P, D, H, V, S, or E, X₂ is selected from I, H, T, E, Q, S, L, K, D, G, Y, V, F, R, A, or P, X₃ is selected from F, E, K, A, R, Q, H, T, D, V, L, G, I, Y, or S, X₄ is selected from S, D, G, F, L, K, I, P, Y, V, A, or T, X₅ is selected from K, T, R, P, Y, S, E, I, L, H, D, F, V, A, or G, X₆ is selected from T, K, A, F, S, G, D, P, H, I, Q, Y, L, or V, X₇ is selected from G, F, H, Y, I, L, T, E, P, R, S, Q, D, K, or A, X₈ is selected from T, E, G, V, S, Q, L, H, R, P, A, F, K, I, or Y, X₉ is selected from Y, I, A, Q, G, E, F, D, K, L, S, T, R, or V, X₁₀ is selected from S, T, G, L, Q, A, I, K, E, H, D, V, R, P, F, or Y, X₁₁ is selected from A, H, Y, V, R, F, G, L, D, E, T, I, K, P, or S, X₁₂ is selected from S, T, I, D, H, V, R, P, L, K, G, Y, V, E, F, or A, X₁₃ is selected from W, Q, F, H, D, S, K, I, T, E, G, R, L, Y, or A, X₁₄ is selected from E, R, P, I, T, A, F, D, S, H, V, K, L, G, Y, or Q, X₁₅ is selected from Y, Q, L, G, V, P, K, E, I, R, H, D, or F, X₁₆ is selected from D, L, E, F, P, R, T, I, S, Y, G, A, V, K, or H, and X₁₇ is selected from F, S, T, Y, K, R, A, H, G, I, V, D, Q, P, E, or L;
      c) amino acid sequences that have 3, 2, or 1 amino acid difference with the amino acid sequence of X₁X₂X₃X₄X₅X₆X₇X₈X₉X₁₀X₁₁X₁₂X₁₃X₁₄X_{15×16}X₁₇ (SEQ ID NO: 4693); wherein X₁ is selected from K, R, L, Y, T, I, Q, A, F, P, D, H, V, S, or E, X₂ is selected from I, H, T, E, Q, S, L, K, D, G, Y, V, F, R, A, or P, X₃ is selected from F, E, K, A, R, Q, H, T, D, V, L, G, I, Y, or S, X₄ is selected from S, D, G, F, L, K, I, P, Y, V, A, or T, X₅ is selected from K, T, R, P, Y, S, E, I, L, H, D, F, V, A, or G, X₆ is selected from T, K, A, F, S, G, D, P, H, I, Q, Y, L, or V, X₇ is selected from G, F, H, Y, I, L, T, E, P, R, S, Q, D, K, or A, X₅ is selected from T, E, G, V, S, Q, L, H, R, P, A, F, K, I, or Y, X₉ is selected from Y, I, A, Q, G, E, F, D, K, L, S, T, R, or V, X₁₀ is selected from S, T, G, L, Q, A, I, K, E, H, D, V, R, P, F, or Y, X₁₁ is selected from A, H, Y, V, R, F, G, L, D, E, T, I, K, P, or S, X₁₂ is selected from S, T, I, D, H, V, R, P, L, K, G, Y, V, E, F, or A, X₁₃ is selected from W, Q, F, H, D, S, K, I, T, E, G, R, L, Y, or A, X₁₄ is selected from E, R, P, I, T, A, F, D, S, H, V, K, L, G, Y, or Q, X₁₅ is selected from Y, Q, L, G, V, P, K, E, I, R, H, D, or F, X₁₆ is selected from D, L, E, F, P, R, T, I, S, Y, G, A, V, K, or H, and X₁₇ is selected from F, S, T, Y, K, R, A, H, G, I, V, D, Q, P, E, or L.
37. The ISVD according to item 34, in which the amino acid sequences of the CDRs (AbM definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 714-1871.
38. The ISVD according to any one of item 34 or 35, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of SEQ ID NO: 1957;
      b) amino acid sequences that have at least 80% amino acid identity with the amino acid sequence of SEQ ID NO: 1957; and
      c) amino acid sequences that have 3, 2, or 1 amino acid difference with the amino acid sequences of SEQ ID NO: 1957;
and
   CDR2 (AbM definition) has an amino acid sequence selected from:
      a) the amino acid sequence of SEQ ID NO: 2127;
      b) amino acid sequences that have at least 80% amino acid identity with the amino acid sequence of SEQ ID NO: 2127;
      c) amino acid sequences that have 3, 2, or 1 amino acid difference with the amino acid sequence of SEQ ID NO: 2127;
and
   CDR3 (AbM definition) has an amino acid sequence selected from:
      a) the amino acid sequence of SEQ ID NOs: 2337;
      b) amino acid sequences that have at least 80% amino acid identity with the amino acid sequence of SEQ ID NO: 2337;
      c) amino acid sequences that have 3, 2, or 1 amino acid difference with the amino acid sequence of SEQ ID NO: 2337.
39. The ISVD according to item 38 in which the amino acid sequences of the CDRs (AbM numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 793, 1505, 1092, 1085, 1078, 1486 or 1083.
40. The ISVD according to any one of the preceding items, in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2096, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2063, CDR3 (AbM numbering) consists of the amino acid sequence of SEQ ID NO: 2337; or in which
   CDR1 (AbM numbering) consists of the amino acid sequence of SEQ ID NO: 2003, CDR2 (AbM numbering) consists of the amino acid sequence of SEQ ID NO: 2117, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2117, CDR3 (AbM numbering) consists of the amino acid sequence of SEQ ID NO: 2337; or in which
   CDR1 (AbM numbering) consists of the amino acid sequence of SEQ ID NO: 2003, CDR2 (AbM numbering) consists of the amino acid sequence of SEQ ID NO: 2062, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2062, CDR3 (AbM numbering) consists of the amino acid sequence of SEQ ID NO: 2337; or in which
   CDR1 (AbM numbering) consists of the amino acid sequence of SEQ ID NO: 2003, CDR2 (AbM numbering) consists of the amino acid sequence of SEQ ID NO: 2097, CDR3 (AbM numbering) consists of the amino acid sequence of SEQ ID NO: 2337; or in which
   CDR1 (AbM numbering) consists of the amino acid sequence of SEQ ID NO: 2003, CDR2 (AbM numbering) consists of the amino acid sequence of SEQ ID NO: 2064, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2064, CDR3 (AbM numbering) consists of the amino acid sequence of SEQ ID NO: 2337; or in which
   CDR1 (AbM numbering) consists of the amino acid sequence of SEQ ID NO: 2003, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2003, CDR2 (AbM numbering) consists of the amino acid sequence of SEQ ID NO: 2061, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2061, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2097, CDR3 (AbM numbering) consists of the amino acid sequence of SEQ ID NO: 2337; or in which
   CDR1 (AbM numbering) consists of the amino acid sequence of SEQ ID NO: 2003, CDR2 (AbM numbering) consists of the amino acid sequence of SEQ ID NO: 2096, CDR3 (AbM numbering) consists of the amino acid sequence of SEQ ID NO: 2337; or in which
   CDR1 (AbM numbering) consists of the amino acid sequence of SEQ ID NO: 2003, CDR2 (AbM numbering) consists of the amino acid sequence of SEQ ID NO: 2063, CDR3 (AbM numbering) consists of the amino acid sequence of SEQ ID NO: 2337; or in which
   CDR1 (AbM numbering) consists of the amino acid sequence of SEQ ID NO: 2003, CDR2 (AbM numbering) consists of the amino acid sequence of SEQ ID NO: 2064, CDR3 (AbM numbering) consists of the amino acid sequence of SEQ ID NO: 2338; or in which
   CDR1 (AbM numbering) consists of the amino acid sequence of SEQ ID NO: 2003, CDR2 (AbM numbering) consists of the amino acid sequence of SEQ ID NO: 2127, CDR3 (AbM numbering) consists of the amino acid sequence of SEQ ID NO: 2338; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2338; or in which
   CDR1 (AbM numbering) consists of the amino acid sequence of SEQ ID NO: 2003, CDR2 (AbM numbering) consists of the amino acid sequence of SEQ ID NO: 2061, CDR3 (AbM numbering) consists of the amino acid sequence of SEQ ID NO: 2338; or in which
   CDR1 (AbM numbering) consists of the amino acid sequence of SEQ ID NO: 1957, CDR2 (AbM numbering) consists of the amino acid sequence of SEQ ID NO: 2061, CDR3 (AbM numbering) consists of the amino acid sequence of SEQ ID NO: 2338; or in which
   CDR1 (AbM numbering) consists of the amino acid sequence of SEQ ID NO: 2003, CDR2 (AbM numbering) consists of the amino acid sequence of SEQ ID NO: 2096, CDR3 (AbM numbering) consists of the amino acid sequence of SEQ ID NO: 2338; or in which
   CDR1 (AbM numbering) consists of the amino acid sequence of SEQ ID NO: 1957, CDR2 (AbM numbering) consists of the amino acid sequence of SEQ ID NO: 2096, CDR3 (AbM numbering) consists of the amino acid sequence of SEQ ID NO: 2338; or in which
   CDR1 (AbM numbering) consists of the amino acid sequence of SEQ ID NO: 1957, CDR2 (AbM numbering) consists of the amino acid sequence of SEQ ID NO: 2063, CDR3 (AbM numbering) consists of the amino acid sequence of SEQ ID NO: 2338; or in which
   CDR1 (AbM numbering) consists of the amino acid sequence of SEQ ID NO: 2003, CDR2 (AbM numbering) consists of the amino acid sequence of SEQ ID NO: 2117, CDR3 (AbM numbering) consists of the amino acid sequence of SEQ ID NO: 2338; or in which
   CDR1 (AbM numbering) consists of the amino acid sequence of SEQ ID NO: 1957, CDR2 (AbM numbering) consists of the amino acid sequence of SEQ ID NO: 2117, CDR3 (AbM numbering) consists of the amino acid sequence of SEQ ID NO: 2338; or in which
   CDR1 (AbM numbering) consists of the amino acid sequence of SEQ ID NO: 2003, CDR2 (AbM numbering) consists of the amino acid sequence of SEQ ID NO: 2062, CDR3 (AbM numbering) consists of the amino acid sequence of SEQ ID NO: 2338; or in which
   CDR1 (AbM numbering) consists of the amino acid sequence of SEQ ID NO: 1957, CDR2 (AbM numbering) consists of the amino acid sequence of SEQ ID NO: 2062, CDR3 (AbM numbering) consists of the amino acid sequence of SEQ ID NO: 2338; or in which
   CDR1 (AbM numbering) consists of the amino acid sequence of SEQ ID NO: 2003, CDR2 (AbM numbering) consists of the amino acid sequence of SEQ ID NO: 2097, CDR3 (AbM numbering) consists of the amino acid sequence of SEQ ID NO: 2338; or in which
   CDR1 (AbM numbering) consists of the amino acid sequence of SEQ ID NO: 2003, CDR2 (AbM numbering) consists of the amino acid sequence of SEQ ID NO: 2063, CDR3 (AbM numbering) consists of the amino acid sequence of SEQ ID NO: 2338; or in which
   CDR1 (AbM numbering) consists of the amino acid sequence of SEQ ID NO: 1957, CDR2 (AbM numbering) consists of the amino acid sequence of SEQ ID NO: 2097, CDR3 (AbM numbering) consists of the amino acid sequence of SEQ ID NO: 2338; or in which
   CDR1 (AbM numbering) consists of the amino acid sequence of SEQ ID NO: 1957, CDR2 (AbM numbering) consists of the amino acid sequence of SEQ ID NO: 2064, CDR3 (AbM numbering) consists of the amino acid sequence of SEQ ID NO: 2338; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2339; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2340; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2341; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2342; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2343; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2344; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2364; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2365; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2366; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2367; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2368; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2369; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2370; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2371; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2372; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2373; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2374; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2375; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2376; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2377; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2378; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2379; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2380; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2381; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2382; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2383; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2384; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2385; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2386; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2387; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2388; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 1890; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2390; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4044; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3917; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4042; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4002; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3999; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3982; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3833; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4009; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4007; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4005; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3929; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4003; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3813; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3835; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3834; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3847; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3920; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3919; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4000; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4035; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4017; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4021; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4013; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4041; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4032; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4027; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4024; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4036; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3935; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4015; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4019; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4038; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4030; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4039; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4011; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4022; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3900; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3921; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4025; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4031; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3819; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3824; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4045; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3817; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3905; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3818; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4028; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4046; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3918; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3822; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3821; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4047; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3820; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2352; or in which
   CDR1 (AbM numbering) consists of the amino acid sequence of SEQ ID NO: 1957, CDR2 (AbM numbering) consists of the amino acid sequence of SEQ ID NO: 2096, CDR3 (AbM numbering) consists of the amino acid sequence of SEQ ID NO: 3812; or in which
   CDR1 (AbM numbering) consists of the amino acid sequence of SEQ ID NO: 2003, CDR2 (AbM numbering) consists of the amino acid sequence of SEQ ID NO: 2061, CDR3 (AbM numbering) consists of the amino acid sequence of SEQ ID NO: 3812; or in which
   CDR1 (AbM numbering) consists of the amino acid sequence of SEQ ID NO: 1957, CDR2 (AbM numbering) consists of the amino acid sequence of SEQ ID NO: 2061, CDR3 (AbM numbering) consists of the amino acid sequence of SEQ ID NO: 3812; or in which
   CDR1 (AbM numbering) consists of the amino acid sequence of SEQ ID NO: 2003, CDR2 (AbM numbering) consists of the amino acid sequence of SEQ ID NO: 2096, CDR3 (AbM numbering) consists of the amino acid sequence of SEQ ID NO: 3812; or in which
   CDR1 (AbM numbering) consists of the amino acid sequence of SEQ ID NO: 2003, CDR2 (AbM numbering) consists of the amino acid sequence of SEQ ID NO: 2063, CDR3 (AbM numbering) consists of the amino acid sequence of SEQ ID NO: 3812; or in which
   CDR1 (AbM numbering) consists of the amino acid sequence of SEQ ID NO: 1957, CDR2 (AbM numbering) consists of the amino acid sequence of SEQ ID NO: 2063, CDR3 (AbM numbering) consists of the amino acid sequence of SEQ ID NO: 3812; or in which
   CDR1 (AbM numbering) consists of the amino acid sequence of SEQ ID NO: 1957, CDR2 (AbM numbering) consists of the amino acid sequence of SEQ ID NO: 2171, CDR3 (AbM numbering) consists of the amino acid sequence of SEQ ID NO: 3940; or in which
   CDR1 (AbM numbering) consists of the amino acid sequence of SEQ ID NO: 1957, CDR2 (AbM numbering) consists of the amino acid sequence of SEQ ID NO: 2058, CDR3 (AbM numbering) consists of the amino acid sequence of SEQ ID NO: 2359; or in which
   CDR1 (AbM numbering) consists of the amino acid sequence of SEQ ID NO: 2003, CDR2 (AbM numbering) consists of the amino acid sequence of SEQ ID NO: 2062, CDR3 (AbM numbering) consists of the amino acid sequence of SEQ ID NO: 3812; or in which
   CDR1 (AbM numbering) consists of the amino acid sequence of SEQ ID NO: 1957, CDR2 (AbM numbering) consists of the amino acid sequence of SEQ ID NO: 2184, CDR3 (AbM numbering) consists of the amino acid sequence of SEQ ID NO: 3940; or in which
   CDR1 (AbM numbering) consists of the amino acid sequence of SEQ ID NO: 1957, CDR2 (AbM numbering) consists of the amino acid sequence of SEQ ID NO: 2171, CDR3 (AbM numbering) consists of the amino acid sequence of SEQ ID NO: 2359; or in which
   CDR1 (AbM numbering) consists of the amino acid sequence of SEQ ID NO: 1957, CDR2 (AbM numbering) consists of the amino acid sequence of SEQ ID NO: 2184, CDR3 (AbM numbering) consists of the amino acid sequence of SEQ ID NO: 2359; or in which
   CDR1 (AbM numbering) consists of the amino acid sequence of SEQ ID NO: 1957, CDR2 (AbM numbering) consists of the amino acid sequence of SEQ ID NO: 2062, CDR3 (AbM numbering) consists of the amino acid sequence of SEQ ID NO: 3812; or in which
   CDR1 (AbM numbering) consists of the amino acid sequence of SEQ ID NO: 2003, CDR2 (AbM numbering) consists of the amino acid sequence of SEQ ID NO: 2117, CDR3 (AbM numbering) consists of the amino acid sequence of SEQ ID NO: 3812; or in which
   CDR1 (AbM numbering) consists of the amino acid sequence of SEQ ID NO: 1957, CDR2 (AbM numbering) consists of the amino acid sequence of SEQ ID NO: 2058, CDR3 (AbM numbering) consists of the amino acid sequence of SEQ ID NO: 3940; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3812; or in which
   CDR1 (AbM numbering) consists of the amino acid sequence of SEQ ID NO: 1957, CDR2 (AbM numbering) consists of the amino acid sequence of SEQ ID NO: 2064, CDR3 (AbM numbering) consists of the amino acid sequence of SEQ ID NO: 3812; or in which
   CDR1 (AbM numbering) consists of the amino acid sequence of SEQ ID NO: 2003, CDR2 (AbM numbering) consists of the amino acid sequence of SEQ ID NO: 2127, CDR3 (AbM numbering) consists of the amino acid sequence of SEQ ID NO: 3812; or in which
   CDR1 (AbM numbering) consists of the amino acid sequence of SEQ ID NO: 2003, CDR2 (AbM numbering) consists of the amino acid sequence of SEQ ID NO: 2097, CDR3 (AbM numbering) consists of the amino acid sequence of SEQ ID NO: 3812; or in which
   CDR1 (AbM numbering) consists of the amino acid sequence of SEQ ID NO: 1957, CDR2 (AbM numbering) consists of the amino acid sequence of SEQ ID NO: 2117, CDR3 (AbM numbering) consists of the amino acid sequence of SEQ ID NO: 3812; or in which
   CDR1 (AbM numbering) consists of the amino acid sequence of SEQ ID NO: 2003, CDR2 (AbM numbering) consists of the amino acid sequence of SEQ ID NO: 2064, CDR3 (AbM numbering) consists of the amino acid sequence of SEQ ID NO: 3812; or in which
   CDR1 (AbM numbering) consists of the amino acid sequence of SEQ ID NO: 1957, CDR2 (AbM numbering) consists of the amino acid sequence of SEQ ID NO: 2097, CDR3 (AbM numbering) consists of the amino acid sequence of SEQ ID NO: 3812; or in which
   CDR1 (AbM numbering) consists of the amino acid sequence of SEQ ID NO: 1957, CDR2 (AbM numbering) consists of the amino acid sequence of SEQ ID NO: 2171, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2058, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2184, CDR3 (AbM numbering) consists of the amino acid sequence of SEQ ID NO: 2337; or in which
   CDR1 (AbM numbering) consists of the amino acid sequence of SEQ ID NO: 2003, CDR2 (AbM numbering) consists of the amino acid sequence of SEQ ID NO: 2183, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3841; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4001; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3998; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3842; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3844; or in which
   CDR1 (AbM numbering) consists of the amino acid sequence of SEQ ID NO: 1957, CDR2 (AbM numbering) consists of the amino acid sequence of SEQ ID NO: 2189, CDR3 (AbM numbering) consists of the amino acid sequence of SEQ ID NO: 2337; or in which
   CDR1 (AbM numbering) consists of the amino acid sequence of SEQ ID NO: 1954, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1961, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1965, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1964, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1958, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2172, CDR3 (AbM numbering) consists of the amino acid sequence of SEQ ID NO: 2337; or in which
   CDR1 (AbM numbering) consists of the amino acid sequence of SEQ ID NO: 1967, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1962, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1963, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1956, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1959, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1955, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1968, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1960, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2182, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2194, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2186, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2054, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2173, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2174, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2191, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2190, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2185, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2055, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2193, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2192, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2195, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2187, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2091, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2053, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2050, CDR3 (AbM numbering) consists of the amino acid sequence of SEQ ID NO: 2337; or in which
   CDR1 (AbM numbering) consists of the amino acid sequence of SEQ ID NO: 1984, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1947, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1987, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1981, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1985, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1988, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1979, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1989, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1973, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1983, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1992, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1991, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1994, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1940, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1953, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2180, CDR3 (AbM numbering) consists of the amino acid sequence of SEQ ID NO: 2337; or in which
   CDR1 (AbM numbering) consists of the amino acid sequence of SEQ ID NO: 1978, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1970, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1982, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1952, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1976, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1972, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1950, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1969, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1951, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1975, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1974, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1948, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1949, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1971, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1986, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1932, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2169, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2059, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2144, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2086, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2087, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2089, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2092, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2167, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2165, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2088, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2077, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2170, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2085, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2090, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2084, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2161, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2083, CDR3 (AbM numbering) consists of the amino acid sequence of SEQ ID NO: 2337; or in which
   CDR1 (AbM numbering) consists of the amino acid sequence of SEQ ID NO: 1995, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2094, CDR3 (AbM numbering) consists of the amino acid sequence of SEQ ID NO: 2337; or in which
   CDR1 (AbM numbering) consists of the amino acid sequence of SEQ ID NO: 1943, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2160, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2159, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2095, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2158, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2157, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2166, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2078, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2163, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2093, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2162, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2156, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2164, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2168, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2072, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2081, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2065, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2056, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2068, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2067, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2179, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2070, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2052, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2181, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2177, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2178, CDR3 (AbM numbering) consists of the amino acid sequence of SEQ ID NO: 2337; or in which
   CDR1 (AbM numbering) consists of the amino acid sequence of SEQ ID NO: 2036, CDR2 (AbM numbering) consists of the amino acid sequence of SEQ ID NO: 2175, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2051, CDR3 (AbM numbering) consists of the amino acid sequence of SEQ ID NO: 2337; or in which
   CDR1 (AbM numbering) consists of the amino acid sequence of SEQ ID NO: 1938, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2073, CDR3 (AbM numbering) consists of the amino acid sequence of SEQ ID NO: 2337; or in which
   CDR1 (AbM numbering) consists of the amino acid sequence of SEQ ID NO: 1980, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2176, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2079, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2075, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2080, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2076, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2074, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2082, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2071, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2069, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2060, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2066, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2057, CDR3 (AbM numbering) consists of the amino acid sequence of SEQ ID NO: 2337; or in which
   CDR1 (AbM numbering) consists of the amino acid sequence of SEQ ID NO: 1977, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1945, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2039, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2032, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1908, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2023, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2034, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2029, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2028, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2026, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1905, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2046, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2044, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2045, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2037, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2042, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2043, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2041, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2038, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2035, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1907, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2033, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2040, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1906, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2030, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1927, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2007, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2006, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1925, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1928, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1936, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1931, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2013, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 3883, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2009, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1934, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2008, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1935, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1933, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1926, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1929, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1930, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2005, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1996, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2000, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1946, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1990, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1942, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1941, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1944, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1993, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1939, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2002, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1999, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1997, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2004, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2001, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1998, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2031, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1937, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1915, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1910, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1911, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1914, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1913, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1918, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2027, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2025, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1909, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2036, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2024, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1912, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1917, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1916, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1919, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2021, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1923, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2014, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2018, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2012, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1922, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1924, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2019, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2010, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1920, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2011, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2020, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2015, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2017, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2016, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 2022, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1921, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2148, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2155, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4034; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3868; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3964; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3869; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4006; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4014; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3865; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3867; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4012; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4004; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2361; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2363; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2362; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3932; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2389; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4008; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3963; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4016; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4020; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3934; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4023; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3816; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4018; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 1893; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4010; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4037; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4026; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4029; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4048; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4040; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3968; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4043; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2357; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3975; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2354; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3871; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3879; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3878; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3876; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3875; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2353; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2356; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3977; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3976; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2355; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3866; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3930; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3969; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3967; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3971; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3965; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2360; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3933; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2358; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3873; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3882; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3880; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2359; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3966; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3881; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3874; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3970; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3855; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3864; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3856; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 4033; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3850; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 1891; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3990; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3994; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3996; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3892; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3992; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3993; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3988; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3991; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3987; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3851; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3958; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3997; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3950; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3995; or in which
   CDR1 (AbM numbering) consists of the amino acid sequence of SEQ ID NO: 1957, CDR2 (AbM numbering) consists of the amino acid sequence of SEQ ID NO: 2153, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2149, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3840; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3849; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3848; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3989; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3845; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3838; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3839; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3846; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3843; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3836; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3852; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3837; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3948; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3949; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3953; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3941; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3858; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3863; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3938; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3859; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3861; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3853; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3946; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3862; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3860; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3937; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3857; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3936; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3940; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3956; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3973; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3944; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3960; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3943; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3942; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3961; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3962; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3959; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3945; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3954; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3939; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3951; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3952; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 1892; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3854; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3974; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2351; or in which
   CDR1 (AbM numbering) consists of the amino acid sequence of SEQ ID NO: 1966, CDR2 (AbM numbering) consists of the amino acid sequence of SEQ ID NO: 2127, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2119, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2121, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2115, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2114, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2118, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2120, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2103, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2108, CDR3 (AbM numbering) consists of the amino acid sequence of SEQ ID NO: 3813; or in which
   CDR1 (AbM numbering) consists of the amino acid sequence of SEQ ID NO: 1957, CDR2 (AbM numbering) consists of the amino acid sequence of SEQ ID NO: 2129, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2104, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2109, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2110, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2113, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2106, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2116, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2125, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2124, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2111, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2125, CDR3 (AbM numbering) consists of the amino acid sequence of SEQ ID NO: 3937; or in which
   CDR1 (AbM numbering) consists of the amino acid sequence of SEQ ID NO: 1957, CDR2 (AbM numbering) consists of the amino acid sequence of SEQ ID NO: 2122, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2126, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2123, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2141, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2112, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2133, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2139, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2132, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2138, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2099, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2134, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2150, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2145, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2147, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2146, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2151, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2154, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2152, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2102, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2140, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2136, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2098, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2107, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2108, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2101, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2128, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2137, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2105, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2130, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2143, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2142, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2131, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2100, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2135, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3981; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3978; or in which
   CDR1 (AbM numbering) consists of the amino acid sequence of SEQ ID NO: 1957, CDR2 (AbM numbering) consists of the amino acid sequence of SEQ ID NO: 2188, CDR3 (AbM numbering) consists of the amino acid sequence of SEQ ID NO: 2337; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3986; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3984; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2350; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3980; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2345; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2348; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3979; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2346; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2349; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3985; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3983; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 2347; or in which
   CDR1 (AbM numbering) consists of the amino acid sequence of SEQ ID NO: 1957, CDR2 (AbM numbering) consists of the amino acid sequence of SEQ ID NO: 2127, CDR3 (AbM numbering) consists of the amino acid sequence of SEQ ID NO: 3877.
41. The ISVD according to any one of items 36 to 39, wherein the ISVD comprises D1 and/or V2 (Kabat numbering).
42. The ISVD according to any one of the preceding items, which amino acid sequence
   i) has 80% amino acid sequence identity with one of the amino acid sequences of SEQ ID NOs: 714-1871, in which for the purposes of determining the degree of amino acid identity, the amino acid residues that form the CDR sequences are disregarded;
   and in which:
   ii) preferably one or more of the amino acid residues at positions 11, 37, 44, 45, 47, 83, 84, 103, 104 and 108 according to the Kabat numbering are chosen from the Hallmark residues mentioned in Table 1.
43. The ISVD according to any one of the preceding items, that essentially consists of a heavy chain variable domain sequence that is derived from a conventional four-chain antibody or that essentially consist of a heavy chain variable domain sequence that is derived from heavy chain antibody.
44. The ISVD according to any one of the preceding items, that essentially consists of a VHH, a humanized VHH, a camelized VH, a domain antibody, a single domain antibody, or a dAb.
45. The ISVD according to any one of the preceding items, which is a humanized ISVD that is chosen from SEQ ID NOs: 714-792, 794-1871 or from the group consisting of amino acid sequences that have more than 80%, preferably more than 90%, more preferably more than 95%, such as 99% or more amino acid sequence identity with at least one of the amino acid sequences of SEQ ID NOs: 714-1871.
46. The ISVD according to item 45, which is a humanized ISVD that is chosen from SEQ ID NO: 1505, 1092, 1085, 1078, 1486, or 1083 or from the group consisting of amino acid sequences that have more than 80%, preferably more than 90%, more preferably more than 95%, such as 99% or more amino acid sequence identity with at least one of the amino acid sequences of SEQ ID NOs: 793, 1505, 1092, 1085, 1078, 1486, or 1083.
47. The ISVD according to any one of the preceding items, in which the amino acid sequence is chosen from the group consisting of SEQ ID NO: 793 and SEQ ID NOs: 714-792, 794-1871.
48. The ISVD according to any one of the preceding items, in which the amino acid sequence is chosen from the group consisting of SEQ ID NO: 793 and SEQ ID NOs: 1505, 1092, 1085, 1078, 1486, or 1083.
49. The ISVD according to any one of the preceding items, wherein the ISVD specifically binds to human PD-1 with a dissociation constant (K_{D}) of 10⁻⁹ to 10⁻¹² moles/litre or less, and preferably 5×10⁻¹⁰ to 10⁻¹² moles/litre or less and more preferably 5×10⁻¹⁰ to 1×10⁻¹¹ moles/litre, such as 4.0×10⁻¹⁰ moles/litre, as determined by Surface Plasmon Resonance, e.g., at 25° C.
50. The ISVD according to any one of the preceding items, wherein the ISVD specifically binds to human PD-1 with a kₒₙ-rate of between 10⁴ M⁻¹ s⁻¹ to about 10⁷ M⁻¹ s⁻¹, preferably between 10⁵ M⁻¹ s⁻¹ and 10⁷ M⁻¹ s⁻¹, more preferably between 10⁵ M⁻¹ s⁻¹ and 10⁶ M⁻¹ s⁻¹, such as between 10⁵ M⁻¹ s⁻¹ and 5×10⁵ M⁻¹ s⁻¹, such as 3.7×10⁵ M⁻¹ s⁻¹, as determined by Surface Plasmon Resonance, e.g., at 25° C.
51. The ISVD according to any one of the preceding items, wherein the ISVD specifically binds to human PD-1 with a k_{off} rate between 10⁻³ s⁻¹ (t1/2=0.69 s) and 10⁻⁷ s⁻¹ (providing a near irreversible complex with a t1/2 of multiple days), preferably between 10³ s⁻¹ and 5×10⁻⁶ s⁻¹, more preferably between 5×10⁻⁴ s⁻¹ and 5×10⁻⁶ s⁻¹, such as between 5×10⁻⁴ s⁻¹ and 10⁵ s⁻¹, such as 1.5×10⁴ s⁻¹, ¹, as determined by Surface Plasmon Resonance, e.g., at 25° C.
52. The ISVD according to any one of the preceding items, wherein the ISVD specifically binds to human and cyno PD-1 and does not bind to CD28, CTLA-4, ICOS, and BTLA or other members of the CD28 receptor family.
53. The ISVD according to any one of the preceding items, wherein the ISVD antagonizes an activity of PD-L1.
54. The ISVD according to any one of the preceding items, wherein the ISVD blocks the interaction of human PD-1 with human PD-L1 with a potency (IC50 value) of 5×10⁻⁸ M or lower, more preferably of 10⁻⁸ M or lower, or even of 10⁹ M or lower, such as between 10⁻¹¹ M and 10⁻⁸ M, between 10⁻¹⁰ M and 10⁻⁹ M, between 10⁻¹⁰ M and 10⁻⁸ M or between 10⁻⁹ M and 10⁻⁸ M, such as 6.07×10⁻⁹ M, for example, as measured in a FACS competition assay on CHO cells expressing human PD-1 with hPD-L1 as a competitor.
55. The ISVD according to any one of the preceding items wherein the ISVD blocks the interaction of cyno PD-1 with cyno PD-L1 with a potency (IC50 value) of 10⁻⁷ M or lower, more preferably of 5×10⁻⁸ M or lower, or even of 10⁻⁸ M or lower, such as between 10⁻¹¹ M and 10⁷ M, between 10⁻¹⁰ M and 10⁻⁹ M, between 10⁻¹⁰ M and 5×10⁻⁸ M or between 10⁻⁸ M and 10⁻⁷ M, such as 1.19×10⁻⁸ M, for example, as measured in a FACS competition assay on CHO cells expressing cyno PD-1 with hPD-L1 as a competitor.
56. The ISVD according to any one of the preceding items, wherein the ISVD blocks the binding of hPD-1 to hPD-L1 by at least 50%, such as at least 60%, 70%, 80%, 90%, 95%, 99% or even more, as determined by ligand competition, AlphaScreen, or competitive binding assays (such as competition ELISA or competition FACS).
57. The ISVD according to any one of the preceding items, wherein the ISVD induces T-cell activation with a potency (EC50 value) of 10⁻⁷ M or lower, more preferably of 5×10⁻⁸ M or lower, or even of 10⁻⁸ M or lower, such as between 10⁻¹¹ M and 10⁻⁷ M, such as between 10⁻¹⁰ M and 10⁻⁸ M, between 10⁻¹⁰ M and 10⁻⁷ M or between 10⁻⁸ M and 10⁻⁷ M, such as 4.32×10⁻⁸ M, 3.24×10⁻⁸ M or 7.1×10⁻⁸ M, as determined in a TT assay.
58. A polypeptide or construct that comprises or essentially consists of one or more ISVDs according to any one of items 1 to 57, and optionally further comprises one or more other groups, residues, moieties or binding units, optionally linked via one or more linkers.
59. The polypeptide or construct according to item 58, in which said one or more other groups, residues, moieties or binding units are amino acid sequences.
60. The polypeptide or construct according to any one of items 58 to 59, in which said one or more linkers are one or more amino acid sequences.
61. The polypeptide or construct according to any one of items 58 to 60, in which said one or more other groups, residues, moieties or binding units are immunoglobulin sequences.
62. The polypeptide or construct according to any one of items 58 to 61, in which said one or more other groups, residues, moieties or binding units are ISVDs.
63. The polypeptide or construct according to any one of items 58 to 62, in which said one or more other groups, residues, moieties or binding units are chosen from the group consisting of VHHs, humanized VHHs, camelized VHs, domain antibodies, single domain antibodies and dAbs.
64. The polypeptide or construct according to any one of items 58 to 63, which is a multivalent construct.
65. The polypeptide or construct according to any one of items 58 to 64, which is a multispecific construct.
66. The polypeptide or construct according to any one of items 58 to 65, in which said one or more other groups, residues, moieties or binding units provide the polypeptide or construct with increased half-life, compared to the ISVD without the one or more other groups, residues, moieties or binding units.
67. The polypeptide or construct according to item 66, in which said one or more other groups, residues, moieties or binding units that provide the polypeptide or construct with increased half-life is chosen from the group consisting of a polyethylene glycol molecule (PEG), serum proteins or fragments thereof, binding units that specifically bind to serum proteins, an Fc portion, and small proteins or peptides that specifically bind to serum proteins.
68. The polypeptide or construct according to item 67, in which said one or more other groups, residues, moieties or binding units that provide the polypeptide or construct with increased half-life is chosen from the group consisting of human serum albumin or fragments thereof.
69. The polypeptide or construct according to item 67, in which said one or more other groups, residues, moieties or binding units that provides the polypeptide or construct with increased half-life are chosen from the group consisting of binding units that specifically bind to serum albumin (such as human serum albumin) or a serum immunoglobulin (such as IgG).
70. The polypeptide or construct according to item 69, in which said one or more other groups, residues, moieties or binding units that provides the polypeptide or construct with increased half-life are chosen from the group consisting of VHHs, humanized VHHs, camelized VHs, domain antibodies, single domain antibodies, or dAbs that specifically bind to serum albumin (such as human serum albumin) or a serum immunoglobulin (such as IgG).
71. The polypeptide or construct according to item 69 or 70, in which said one or more other groups, residues, moieties or binding units that provides the polypeptide or construct with increased half-life is an ISVD that specifically binds human serum albumin.
72. The polypeptide or construct according to item 71, wherein said ISVD that specifically binds human serum albumin essentially consists of 4 framework regions (FR1 to FR4, respectively) and 3 complementarity determining regions (CDR1 to CDR3 respectively), in which:
   CDR1 (AbM numbering) has an amino acid sequence selected from:
      a) the amino acid sequence of SEQ ID NO: 2436;
      b) amino acid sequences that have at least 80% amino acid identity with the amino acid sequence of SEQ ID NO: 2436; and
      c) amino acid sequences that have 3, 2, or 1 amino acid difference with the amino acid sequences of SEQ ID NO: 2436;
and
   CDR2 (AbM numbering) has an amino acid sequence selected from:
      a) the amino acid sequence of SEQ ID NO: 2438;
      b) amino acid sequences that have at least 80% amino acid identity with the amino acid sequence of SEQ ID NO: 2438;
      c) amino acid sequences that have 3, 2, or 1 amino acid difference with the amino acid sequence of SEQ ID NO: 2438;
and
   CDR3 (AbM numbering) has an amino acid sequence selected from:
      a) the amino acid sequence of SEQ ID NOs: 2440;
      b) amino acid sequences that have at least 80% amino acid identity with the amino acid sequence of SEQ ID NO: 2440;
      c) amino acid sequences that have 3, 2, or 1 amino acid difference with the amino acid sequence of SEQ ID NO: 2440.
73. The polypeptide or construct according to item 72, wherein CDR1 consists of the amino acid sequence of SEQ ID NO: 2436, CDR2 consists of the amino acid sequence of SEQ ID NO: 2438, and CDR3 consists of the amino acid sequence of SEQ ID NO: 2440.
74. The polypeptide or construct according to any one of items 71 to 73, wherein said ISVD that specifically binds human serum albumin is selected from the group consisting of ALB8 (SEQ ID NO: 2401), ALB23 (SEQ ID NO: 2402), ALBX00001 (SEQ ID NO: 2415) and ALB23002 (SEQ ID NO: 2416), preferably the ISVD binding to human serum albumin is ALB23002 (SEQ ID NO: 2416).
75. The polypeptide or construct according to anyone of items 58 to 74, wherein said linker is chosen from the group consisting of SEQ ID NOs: 2418 to 2434.
76. The polypeptide or construct according to any one of items 58 to 75, further comprising a C-terminal extension.
77. The polypeptide or construct according to item 76, wherein said C-terminal extension is a C-terminal extension (X)ₙ, in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I).
78. A nucleic acid that encodes an ISVD according to any one of items 1 to 57, or a polypeptide according to any one of items 58 to 77.
79. The nucleic acid according to item 78, that is in the form of a genetic construct.
80. A (non-human) host or host cell that expresses, or that under suitable circumstances is capable of expressing, an ISVD according to any one of one items 1 to 57, or a polypeptide according to any one of items 58 to 77; and/or that comprises the nucleic acid according to any one of items 78 or 79.
81. A method for producing an ISVD according to any one of items 1 to 57, or a polypeptide according to any one of items 58 to 77, at least comprising the steps of:
   a) expressing, in a suitable (non-human) host organism or a host cell or in another suitable expression system, a nucleic acid according to any one of items 78 or 79;
   optionally followed by:
   b) isolating and/or purifying the ISVD according to any one of items 1 to 57, or the polypeptide according to any one of items 58 to 77.
82. A method for producing an ISVD according to any one of items 1 to 57, or a polypeptide according to any one of items 58 to 77, said method at least comprising the steps of:
   a) cultivating and/or maintaining a (non-human) host or host cell according to item 81 under conditions that are such that said non-human host or host cell expresses and/or produces at least one ISVD according to any one of items 1 to 57, or at least one polypeptide according to any one of items 58 to 77;
   optionally followed by:
   b) isolating and/or purifying the ISVD according to any one of items 1 to 57, or polypeptide according to any one of items 58 to 77.
83. A composition comprising at least one ISVD according to any one of items 1 to 57, at least one polypeptide or construct according to any one of items 58 to 77, or at least one nucleic acid according to any one of items 78 or 79.
84. The composition according to item 83, which is a pharmaceutical composition.
85. The composition according to any one of items 83 or 84, which is a pharmaceutical composition, that further comprises at least one pharmaceutically acceptable carrier, diluent or excipient and/or adjuvant, and that optionally comprises one or more further pharmaceutically active polypeptides and/or compounds.
86. The ISVD according to any one of items 1 to 57, the polypeptide or construct according to any one of items 58 to 77, or the composition according to any one of items 83 to 85, for use as a medicament.
87. The ISVD according to any one of items 1 to 57, the polypeptide or construct according to any one of items 58 to 77, or the composition according to any one of items 83 to 85, for use in the diagnosis, prevention and/or treatment of at least one disease and/or disorder.
88. The ISVD according to any one of items 1 to 57, the polypeptide or construct according to any one of items 58 to 77, or the composition according to any one of items 83 to 85, for use in the diagnosis, prevention and/or treatment of at least one disease and/or disorder that is associated with PD-1, with its biological or pharmacological activity, and/or with the biological pathways or signalling in which PD-1 is involved.
89. The ISVD according to any one of items 1 to 57, the polypeptide or construct according to any one of items 58 to 77, or the composition according to any one of items 83 to 85, for use in the diagnosis, prevention and/or treatment of cancer.
90. A method for the diagnosis, prevention and/or treatment of at least one disease and/or disorder, comprising the administration, to a subject in need thereof, of a pharmaceutically active amount of an ISVD according to any one of items 1 to 57, a polypeptide or construct according to any one of items 58 to 77, or a composition according to any one of items 83 to 85.
91. The method according to item 90, for the diagnosis, prevention and/or treatment of at least one disease or disorder that is associated with PD-1, with its biological or pharmacological activity, and/or with the biological pathways or signalling in which PD-1 is involved, said method comprising administering, to a subject in need thereof, a pharmaceutically active amount of at least one ISVD according to any one of items 1 to 57, a polypeptide or construct according to any one of items 58 to 77, or composition according to any one of items 83 to 85.
92. The method according to any one of items 90 or 91, for the diagnosis, prevention and/or treatment of cancer, said method comprising administering, to a subject in need thereof, a pharmaceutically active amount of at least one ISVD according to any one of items 1 to 57, a polypeptide or construct according to any one of items 58 to 77, or a composition according to any one of items 83 to 85.

### 7.3 Embodiment 3, Family A0294037F03

1. An immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1 protein, wherein at least one amino acid residue located in at least one of the CDRs is capable of interacting with at least one amino acid residue in at least one, preferably in each, of the following stretches (of the ectodomain) of human PD-1 protein, when numbered in accordance with SEQ ID NO: 4703: W32, Y68-M70, K78, and 1126-R139, preferably when the ISVD and human PD-1 protein are mixed in PBS1× buffer and crystallized by hanging drop vapor diffusion using 1 M Na Acetate, 1M (NH4)₂HPO₄, pH 4.5 at 19° C.
2. The ISVD according to item 1, wherein the at least one amino acid residue in at least one, preferably in each stretch (of the ectodomain) of human PD-1 protein is selected from:
   For stretch Y68-M70: Y68, and M70;
   For stretch I126-R139: I126, L128, P130, A132, Q133, I134, K135, E136, and R139, when numbered in accordance with SEQ ID NO: 4703.
3. The ISVD according to item 1 or 2, wherein at least one amino acid residue located in at least one of the CDRs is capable of interacting with all of following amino acid residues in stretch Y68-M70 (of the ectodomain) of human PD-1 protein, when numbered in accordance with SEQ ID NO: 4703: Y68, and M70.
4. The ISVD according to any one of the preceding items, wherein at least one amino acid residue located in at least one of the CDRs is capable of interacting with at least two, at least three, at least four, at least five, at least six, at least seven, at least eight, or all of following amino acid residues in stretch I126-R139 (of the ectodomain) of human PD-1 protein when numbered in accordance with SEQ ID NO: 4703: I126, L128, P130, A132, Q133, I134, K135, E136, and R139, and optionally K131.
5. The ISVD according to any one of the preceding items, wherein the at least one amino acid residue in each stretch (of the ectodomain) of human PD-1 protein is K131, when numbered in accordance with SEQ ID NO: 4703.
6. The ISVD according to any one of the preceding items, wherein at least one amino acid residue located in at least one of the CDRs is capable of interacting with at least two, at least three, at least four, at least five, at least six, at least seven, at least eight, or all of following amino acid residues in stretch I126-R139 (of the ectodomain) of human PD-1 protein when numbered in accordance with SEQ ID NO: 4703: I126, L128, P130, K131, A132, Q133, I134, K135, E136, and R139.
7. The ISVD according to any one of the preceding items, wherein at least one amino acid residue located in at least one of the CDRs is capable of forming hydrogen bonds with at least one, preferably all, of the following amino acid residues of human PD-1 protein, when numbered in accordance with SEQ ID NO: 4703: A132, and Q133, preferably when the ISVD and human PD-1 protein are mixed in PBS1× buffer and crystallized by hanging drop vapor diffusion using 1 M Na Acetate, 1M (NH₄)₂HPO₄, pH 4.5 at 19° C.
8. The ISVD according to any one of the preceding items, wherein at least one amino acid residue located in at least one of the CDRs is capable of forming hydrogen bonds with at least one, preferably all, of the following amino acid residues of human PD-1 protein, when numbered in accordance with SEQ ID NO: 4703: M70, K78, K131, A132, Q133, I134, K135, and E136, and optionally K131, preferably when the ISVD and human PD-1 protein are mixed in PBS1× buffer and crystallized by hanging drop vapor diffusion using 1 M Na Acetate, 1M (NH4)₂HPO₄, pH 4.5 at 19° C.
9. The ISVD according to any one of the preceding items, wherein the at least one amino acid residue located in at least one of the CDRs is capable of interacting with an (discontinuous) epitope on human PD-1 comprising at least one amino acid residue in the following stretch (of the ectodomain) of human PD-1 protein Y68-M70: when numbered in accordance with SEQ ID NO: 4703.
10. The ISVD according to item 9, wherein the at least one amino acid residue in said stretch (of the ectodomain) of human PD-1 protein is selected from: Y68, and M70, when numbered in accordance with SEQ ID NO: 4703.
11. The ISVD according to any one of the preceding items, wherein the at least one amino acid residue located in at least one of the CDRs is capable of interacting with an (discontinuous) epitope on human PD-1 comprising at least one amino acid residue in the following stretch (of the ectodomain) of human PD-1 protein I126-R139, when numbered in accordance with SEQ ID NO: 4703.
12. The ISVD according to item 11, wherein the at least one amino acid residue in said stretch (of the ectodomain) of human PD-1 protein is selected from:
   I126, L128, P130, A132, Q133, I134, K135, E136, R139 and optionally K131, when numbered in accordance with SEQ ID NO: 4703.
13. The ISVD according to any one of the preceding items, wherein at least one amino acid residue located in at least one of the CDRs is capable of interacting with at least one, at least two, at least three, at least four, at least five, at least six, at least seven, at least ten, at least fifteen, preferably all, of the following amino acid residues of human PD-1 protein, when numbered in accordance with SEQ ID NO: 4703: W32, Y68, M70, I126, L128, P130, A132, Q133, I134, K135, E136, R139, and E136 and optionally K131, preferably when the ISVD and human PD-1 protein are mixed in PBS1× buffer and crystallized by hanging drop vapor diffusion using 1 M Na Acetate, 1M (NH₄)₂HPO₄, pH 4.5 at 19° C.
14. The ISVD according to any one of the preceding items, wherein the amino acid residue located in at least one of the CDRs which are capable of interacting with amino acid residues of human PD-1 protein are located in CDR1, CDR2 and/or CDR3.
15. The ISVD according to any one of the preceding items, wherein:
   the amino acid residue at position 100 (Kabat numbering) is capable of interacting, preferably by forming a hydrogen bond, with Q133 in PD-1;
   the amino acid residue at position 100b (Kabat numbering) is capable of interacting, preferably by forming a hydrogen bond, with A132 in PD-1;
and optionally wherein:
   the amino acid residue at position 99 (Kabat numbering) is capable of interacting, preferably by forming a hydrogen bond, with K135 in PD-1;
   the amino acid residue at position 54 (Kabat numbering) is capable of interacting, preferably by forming a hydrogen bond, with M70 in PD-1;
   the amino acid residue at position 32 (Kabat numbering) is capable of interacting, preferably by forming a hydrogen bond, with 1134 in PD-1;
   the amino acid residue at position 54 (Kabat numbering) is capable of interacting, preferably by forming a hydrogen bond, with E136 in PD-1;
   the amino acid residue at position 52 (Kabat numbering) is capable of interacting, preferably by forming a hydrogen bond, with E136 in PD-1;
   the amino acid residue at position 57 (Kabat numbering) is capable of interacting, preferably by forming a hydrogen bond, with K78 in PD-1; and/or.
   the amino acid residue at position 54 (Kabat numbering) is capable of interacting, preferably by forming a hydrogen bond, with K131 in PD-1;
   preferably when the ISVD and human PD-1 protein are mixed in PBS1× buffer and crystallized by hanging drop vapor diffusion using 1 M Na Acetate, 1M (NH₄)₂HPO₄, pH 4.5 at 19° C.
16. The ISVD according to any one of the preceding items, wherein two amino acids are considered to interact when the distance between them is 4.0 Å or less.
17. The ISVD according to any one of the preceding items, wherein the interacting amino acid residue(s) in the ISVD and in human PD-1 protein are identified by X-ray crystallography by:
   Mixing human PD-1 protein, and the ISVD in PBS1× buffer;
   Concentrating the protein complex to 12 mg/ml;
   Crystallizing by hanging drop vapor diffusion using 1 M Na Acetate, 1M (NH₄)₂HPO₄, pH 4.5 at 19° C.;
   Collecting X-ray diffraction data;
   Processing the collected data, preferably as described in Example 13.
18. The ISVD according to any one of the preceding items, wherein the (discontinuous) epitope comprises or consists of the following amino acid residues of human PD-1 protein, when numbered in accordance with SEQ ID NO: 4703: W32, Y68, M70, I126, L128, P130, K131, A132, Q133, I134, K135, E136, R139.
19. The ISVD according to any one of the preceding items, wherein the ISVD cross-blocks the binding to human PD-1 protein of a polypeptide having an amino acid sequence selected from SEQ ID NOs: 2857-3662, preferably wherein the ISVD cross-blocks the binding to human PD-1 protein of a polypeptide having an amino acid sequence selected from SEQ ID NO: 2859, or wherein the binding of the ISVD to human PD-1 protein is cross-blocked by a polypeptide having an amino acid sequence selected from SEQ ID NOs: 2857-3662.
20. An immunoglobulin single variable domain (ISVD) specifically binding human PD-1 protein, that essentially consists of 4 framework regions (FR1 to FR4, respectively) and 3 complementarity determining regions (CDR1 to CDR3, respectively), wherein the ISVD comprises an amino acid residue capable of forming hydrogen bonds at at least one, preferably at all, of the following positions: 100, 100b (Kabat numbering) and optionally further at position 32, 52, 54, 57, 99 (Kabat numbering, preferably wherein the ISVD comprises at least one, preferably all of the following amino acid residues at the following positions: S100, Y100b (Kabat numbering) and optionally N32, S52, N54, T57, S99 (Kabat numbering).
21. The ISVD according to any one of the preceding items, wherein the ISVD comprises an amino acid residue capable of forming hydrogen bonds at position 45 (Kabat numbering, preferably wherein the ISVD comprises R45 (Kabat numbering).
22. The ISVD according to any one of the preceding items, wherein the ISVD comprises at least one, preferably all of the following amino acid residues at the following positions (Kabat numbering): N32, and A33, preferably at least N32.
23. The ISVD according to any one of the preceding items, wherein the ISVD comprises at least one, preferably all of the following amino acid residues at the following positions (Kabat numbering): L50, S52, N54, I56, T57, and Y58, preferably at least S52, N54, and T57.
24. The ISVD according to any one of the preceding items, wherein the ISVD comprises at least one, preferably all of the following amino acid residues at the following positions (Kabat numbering): E95, Y97, S99, S100, W100a, Y100b, preferably at least S99, S100, and Y100b.
25. The ISVD according to any one of the preceding items, wherein:
   the CDR1 (AbM definition) consists of the following amino acid sequence X₁X₂X₃X₄X₅X₆NAMA (SEQ ID NO: 4699); wherein
      X₁ is selected from any amino acid, preferably X₁ is selected from G, T, D, F, S, R, E, H, I, A, V, or K;
      X₂ is selected from any amino acid, preferably X₂ is selected from R, M, D, H, Y, F, V, L, Q, K, P, A, I, or G;
      X₃ is selected from any amino acid, preferably X₃ is selected from M, F, R, S, I, D, V, K, T, Y, A, E, or G;
      X₄ is selected from any amino acid, preferably X₄ is selected from H, R, S, E, P, G, Q, K, A, D, T, V, I, F, Y, or L;
      X₅ is selected from any amino acid, preferably X₅ is selected from S, T, K, G, I, Y, H, F, D, E, V, P, and L;
      X₆ is selected from any amino acid, preferably X₆ is selected from I, V, S, K, E, D, Q, R, P, G, T, L, or F; and/or
   the CDR2 (AbM definition) consists of the following amino acid sequence LX₁SX₂X₃NX₄ITY (SEQ ID NO: 4700); wherein:
      X₁ is selected from any amino acid, preferably X₁ is selected from I, G, Y, A, Q, H, T, L, S, D, R, K, or P;
      X₂ is selected from any amino acid, preferably X₂ is selected W, L, K, S, Q, V, D, L, H, T, E, I, A, R, P, Y, G, or F;
      X₃ is selected from any amino acid, preferably X₃ is selected from G, L, T, Y, R, V, P, Q, F, A, and S;
      X₄ is selected from any amino acid, preferably X₄ is selected from G, Q, F, S, I, H, R, E, V, P, L, D, T, or A; and/or
   the CDR3 (AbM definition) consists of the following amino acid sequence EX₁Y X₂SSWYX₃X₄ (SEQ ID NO: 4701); wherein:
      X₁ is selected from any amino acid, preferably X₁ is selected from S, F, A, Y, E, I, D, K, G, R,P, Q, T, H, or L;
      X₂ is selected from any amino acid, preferably X₂ is selected G, L, I, H, S, V, R, P, E, A, K, Y, T, F, A, D, H, or R;
      X₃ is selected from any amino acid, preferably X₃ is selected from D, N, A, S, L, K, T, F, I, G, Y, R, V, E, or H, or is absent;
      X₄ is selected from any amino acid, preferably X₄ is selected from S, D, V, H, A, L, E, T, Y, K, F, I, Q, R, G, or P, or is absent.
26. The ISVD according to any one of the preceding items, wherein at least one, preferably all of the following amino acid residues of the ISVD at the following positions: S99, S100, Y100b (Kabat numbering), optionally N32, S52, N54, T57 (Kabat numbering), and further optionally R45, are capable of forming hydrogen bonds with at least one of the amino acid residues of human PD-1 protein, preferably when the ISVD and human PD-1 protein are mixed in PBS1× buffer and crystallized by hanging drop vapor diffusion using 1 M Na Acetate, 1M (NH₄)₂HPO₄, pH 4.5 at 19° C.
27. The ISVD according to any one of the preceding items, wherein at least one, preferably all, of the following amino acid residues of the ISVD at the following positions: N32, S52, N54, T57, S99, S100, and Y100b, (Kabat numbering) is capable of interacting with at least one, preferably all of the following amino acid residues of human PD-1 protein, when numbered in accordance with SEQ ID NO: 4703: M70, K78, A132, Q133, I134, K135, and E136, preferably wherein:
   S99 in the ISVD (Kabat numbering) interacts, preferably by forming a hydrogen bond, with K135 in PD-1;
   S100 in the ISVD (Kabat numbering) interacts, preferably by forming a hydrogen bond, with Q133 in PD-1;
   Y100b in the ISVD (Kabat numbering) interacts, preferably by forming a hydrogen bond, with A132 in PD-1;
   N54 in the ISVD (Kabat numbering) interacts, preferably by forming a hydrogen bond, with M70 in PD-1;
   N32 in the ISVD (Kabat numbering) interacts, preferably by forming a hydrogen bond, with 1134 in PD-1;
   N54 in the ISVD (Kabat numbering) interacts, preferably by forming a hydrogen bond, with E136 in PD-1; and/or
   S52 in the ISVD (Kabat numbering) interacts, preferably by forming a hydrogen bond, with E136 in PD-1; and/or
T57 in the ISVD (Kabat numbering) interacts, preferably by forming a hydrogen bond, with K78 in PD-1; preferably when the ISVD and human PD-1 protein are mixed in PBS1× buffer and crystallized by hanging drop vapor diffusion using 1 M Na Acetate, 1M (NH₄)₂HPO₄, pH 4.5 at 19° C.
28. The ISVD according to any one of the preceding items, wherein R45 (Kabat numbering) is capable of interacting with K131 of human PD-1 protein, when numbered in accordance with SEQ ID NO: 4703, preferably wherein R45 in the ISVD (Kabat numbering) interacts, preferably by forming a hydrogen bond, with K131 in PD-1;
   preferably when the ISVD and human PD-1 protein are mixed in PBS1× buffer and crystallized by hanging drop vapor diffusion using 1 M Na Acetate, 1M (NH₄)₂HPO₄, pH 4.5 at 19° C.
29. The ISVD according to any one of the preceding items, wherein the ISVD comprises at least one, preferably all of the following amino acid residues at the following positions (Kabat numbering): S100, and Y100b.
30. The ISVD according to any one of the preceding items, wherein the ISVD comprises at least one, at least two, at least three, at least four, at least five, at least six, at least seven, preferably all of the following amino acid residues at the following positions (Kabat numbering): N32, S52, N54, T57, S99, S100, and Y100b, and optionally R45.
31. The ISVD according to any one of the preceding items, wherein the ISVD comprises at least one, at least two, at least three, at least four, at least five, at least six, at least seven, at least eight, at least nine, at least ten, at least eleven, at least twelve, at least thirteen, preferably all of the following amino acid residues at the following positions (Kabat numbering): N32, A33, L50, S52, N54, I56, T57, Y58, E95, Y97, S99, S100, W100a, and Y100b.
32. The ISVD according to any one of the preceding items, wherein the ISVD comprises at least one, at least two, at least three, at least four, at least five, at least six, at least seven, at least eight, at least nine, at least ten, at least eleven, at least twelve, at least thirteen, at least fourteen, at least fifteen, at least sixteen preferably all of the following amino acid residues at the following positions (Kabat numbering): N32, A33, F37, R45, F47, L50, S52, N54, I56, T57, Y58, E95, Y97, S99, S100, W100a, Y100b.
33. The ISVD according to any one of the preceding items, in which S100, and Y100b (Kabat numbering) of the ISVD are capable of interacting with at least one of the following amino acids of human PD-1 protein, when numbered in accordance with SEQ ID NO: 4703: A132, and Q133, preferably when the ISVD and human PD-1 protein are mixed in PBS1× buffer and crystallized by hanging drop vapor diffusion using 1 M Na Acetate, 1M (NH₄)₂HPO₄, pH 4.5 at 19° C.
34. The ISVD according to any one of the preceding items, in which N32, S52, N54, T57, S99, S100, and Y100b (Kabat numbering) of the ISVD are capable of interacting with at least one of the following amino acids of human PD-1 protein, when numbered in accordance with SEQ ID NO: 4703: W32, Y68, M70, K78, I126, L128, P130, A132, Q133, I134, K135, E136, and R139, preferably when the ISVD and human PD-1 protein are mixed in PBS1× buffer and crystallized by hanging drop vapor diffusion using 1 M Na Acetate, 1M (NH₄)₂HPO₄, pH 4.5 at 19° C.
35. The ISVD according to any one of the preceding items, in which N32, R45, S52, N54, T57, S99, S100, and Y100b (Kabat numbering) of the ISVD are capable of interacting with at least one of the following amino acids of human PD-1 protein, when numbered in accordance with SEQ ID NO: 4703: W32, Y68, M70, K78, I126, L128, P130, K131, A132, Q133, I134, K135, E136, R139, and E136, preferably when the ISVD and human PD-1 protein are mixed in PBS1× buffer and crystallized by hanging drop vapor diffusion using 1 M Na Acetate, 1M (NH₄)₂HPO₄, pH 4.5 at 19° C.
36. The ISVD according to any one of the preceding items, in which N32, A33, F37, R45, F47, L50, S52, N54, I56, T57, Y58, E95, Y97, S99, S100, W100a, Y100b (Kabat numbering) of the ISVD are capable of interacting with the following amino acids of human PD-1 protein, when numbered in accordance with SEQ ID NO: 4703: W32, Y68, M70, K78, I126, L128, P130, K131, A132, Q133, I134, K135, E136, and R139, preferably when the ISVD and human PD-1 protein are mixed in PBS1× buffer and crystallized by hanging drop vapor diffusion using 1 M Na Acetate, 1M (NH₄)₂HPO₄, pH 4.5 at 19° C.
37. The ISVD according to any one of the preceding items, wherein two amino acids are considered to interact when the distance between them is 4.0 Å or less.
38. The ISVD according to any one of the preceding items, wherein the interacting amino acid residue(s) in the ISVD and in human PD-1 protein are identified by X-Ray crystallography by:
   Mixing human PD-1 protein, and the ISVD in PBS1× buffer;
   Concentrating the protein complex to 8 mg/ml;
   Crystallizing by hanging drop vapor diffusion using 1 M Na Acetate, 1M (NH₄)₂HPO₄, pH 4.5 at 19° C.;
   Collecting X-ray diffraction data;
   Processing the collected data,
preferably as described in Example 13.
39. An immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1, wherein the ISVD cross-blocks the binding to human PD-1 protein of a polypeptide with the amino acid sequence of SEQ ID Nos: 2857-3662 or wherein the ISVD is cross-blocked from binding to human PD-1 protein by a polypeptide with the amino acid sequence of SEQ ID Nos: 2857-3662.
40. The ISVD according to any one of the preceding items, wherein the ISVD competes with a polypeptide with the amino acid sequence of SEQ ID NO: 2857-3662 for binding human PD-1 protein, as measured in a competition assay, such as a FACS competition assay.
41. An immunoglobulin single variable domain (ISVD) specifically binding human PD-1, that essentially consists of 4 framework regions (FR1 to FR4, respectively) and 3 complementarity determining regions (CDR1 to CDR3, respectively), in which:
   CDR1 (AbM numbering) has an amino acid sequence selected from:
      a) the amino acid sequence of X₁X₂X₃X₄X₅X₆X₇X₈MA (SEQ ID NO: 4608), wherein X₁ is selected from G, T, D, F, S, R, E, H, I, A, V, or K, X₂ is selected from R, M, D, H, Y, F, V, L, Q, K, P, A, I, or G, wherein X₃ is selected from M, F, R, S, I, D, V, K, T, Y, A, E, or G, X₄ is selected from H, R, S, E, P, G, Q, K, A, D, T, V, I, F, Y, or L, X₅ is selected from S, T, K, G, I, Y, H, F, D, E, V, P, and L, X₆ is selected from I, V, S, K, E, D, Q, R, P, G, T, L, or F, X₇ is selected from N, G, V, E, L, I, Y, Q, T, F, P, or H, and X₈ is selected from A, D, E, I, H, S, V, L, K, G, T, or Y;
      b) amino acid sequences that have at least 80% amino acid identity with the amino acid sequence of X₁X₂X₃X₄X₅X₆X₇X&MA (SEQ ID NO: 4608), wherein X₁ is selected from G, T, D, F, S, R, E, H, I, A, V, or K, X₂ is selected from R, M, D, H, Y, F, V, L, Q, K, P, A, I, or G, wherein X₃ is selected from M, F, R, S, I, D, V, K, T, Y, A, E, or G, X₄ is selected from H, R, S, E, P, G, Q, K, A, D, T, V, I, F, Y, or L, X₅ is selected from S, T, K, G, I, Y, H, F, D, E, V, P, and L, X₆ is selected from I, V, S, K, E, D, Q, R, P, G, T, L, or F, X₇ is selected from N, G, V, E, L, I, Y, Q, T, F, P, or H, and X₈ is selected from A, D, E, E, I, H, S, V, L, K, G, T, or Y; and
      c) amino acid sequences that have 3, 2, or 1 amino acid difference with the amino acid sequences of X₁X₂X₃X₄X₅X₆X₇X₈MA (SEQ ID NO: 4608), wherein X₁ is selected from G, T, D, F, S, R, E, H, I, A, V, or K, X₂ is selected from R, M, D, H, Y, F, V, L, Q, K, P, A, I, or G, wherein X₃ is selected from M, F, R, S, I, D, V, K, T, Y, A, E, or G, X₄ is selected from H, R, S, E, P, G, Q, K, A, D, T, V, I, F, Y, or L, X₅ is selected from S, T, K, G, I, Y, H, F, D, E, V, P, and L, X₆ is selected from I, V, S, K, E, D, Q, R, P, G, T, L, or F, X₇ is selected from N, G, V, E, L, I, Y, Q, T, F, P, or H, and X₈ is selected from A, D, E, E, I, H, S, V, L, K, G, T, or Y;
and
   CDR2 (AbM numbering) has an amino acid sequence selected from:
      a) the amino acid sequence of X₁X₂X₃X₄X₅X₆X₇X₈X₉X₁₀ (SEQ ID NO: 4609), wherein X₁ is selected from L, V, I, T, F, E, Q, D, R, G, S, A, P, H, or K, X₂ is selected from I, G, Y, A, Q, H, T, L, S, D, R, K, or P, and X₃ is selected from S, G, L, V, K, F, I, T, E, A, D, H, P, Q, or R, X₄ is selected from W, L, K, S, Q, V, D, L, H, T, E, I, A, R, P, Y, G, or F, X₅ is selected from G, L, T, Y, R, V, P, Q, F, A, and S, X₆ is selected from N, D, H, T, A, F, E, S, V, Q, I, L, R, Y, G, X₇ is selected from G, Q, F, S, I, H, R, E, V, P, L, D, T, or A, X₈ is selected from I, Q, T, L, P, V, Y, H, D, R, E, A, G, F, S, or K, X₉ is selected from T, L, D, Q, I, S, G, F, E, A, Y, P, V, or K, and X₁₀ is selected from Y, P, R, T, G, A, S, Q, D, E, L, K, I, F, V, or H;
      b) amino acid sequences that have at least 80% amino acid identity with the amino acid sequence of X₁X₂X₃X₄X₅X₆X₇X₈X₉X₁₀ (SEQ ID NO: 4609), wherein X₁ is selected from L, V, I, T, F, E, Q, D, R, G, S, A, P, H, or K, X₂ is selected from I, G, Y, A, Q, H, T, L, S, D, R, K, or P, and X₃ is selected from S, G, L, V, K, F, I, T, E, A, D, H, P, Q, or R, X₄ is selected from W, L, K, S, Q, V, D, L, H, T, E, I, A, R, P, Y, G, or F, X₅ is selected from G, L, T, Y, R, V, P, Q, F, A, and S, X₆ is selected from N, D, H, T, A, F, E, S, V, Q, I, L, R, Y, G, X₇ is selected from G, Q, F, S, I, H, R, E, V, P, L, D, T, or A, X₈ is selected from I, Q, T, L, P, V, Y, H, D, R, E, A, G, F, S, or K, X₉ is selected from T, L, D, Q, I, S, G, F, E, A, Y, P, V, or K, and X₁₀ is selected from Y, P, R, T, G, A, S, Q, D, E, L, K, I, F, V, or H
      c) amino acid sequences that have 3, 2, or 1 amino acid difference with the amino acid sequence of X₁X₂X₃X₄X₅X₆X₇X₈X₉X₁₀ (SEQ ID NO: 4609), wherein X₁ is selected from L, V, I, T, F, E, Q, D, R, G, S, A, P, H, or K, X₂ is selected from I, G, Y, A, Q, H, T, L, S, D, R, K, or P, and X₃ is selected from S, G, L, V, K, F, I, T, E, A, D, H, P, Q, or R, X₄ is selected from W, L, K, S, Q, V, D, L, H, T, E, I, A, R, P, Y, G, or F, X₅ is selected from G, L, T, Y, R, V, P, Q, F, A, and S, X₆ is selected from N, D, H, T, A, F, E, S, V, Q, I, L, R, Y, G, X₇ is selected from G, Q, F, S, I, H, R, E, V, P, L, D, T, or A, X₈ is selected from I, Q, T, L, P, V, Y, H, D, R, E, A, G, F, S, or K, X₉ is selected from T, L, D, Q, I, S, G, F, E, A, Y, P, V, or K, and X₁₀ is selected from Y, P, R, T, G, A, S, Q, D, E, L, K, I, F, V, or H;
and
   CDR3 (AbM numbering) has an amino acid sequence selected from:
      a) the amino acid sequence of X₁X₂X₃X₄X₅X₆X₇X₈X₉X₁₀ (SEQ ID NO: 4610), wherein X₁ is selected from E, P, D, A, K, I, Y, H, R, F, S, T, V, G, or Q, X₂ is selected S, F, A, Y, E, I, D, K, G, R, P, Q, T, H, or L, and X₃ is selected Y, D, F, E, R, K, I, T, A, Q, S, V, H, G, or P, X₄ is selected from G, L, I, H, S, V, R, P, E, A, K, Y, T, F, A, D, H, or R, X₅ is selected from S, D, K, Y, A, L, I, P, V, Q, E, R, T, G, or F, X₆ is selected from S, T, K, P, H, I, G, V, Q, R, D, L, or E, X₇ is selected from W, C, P, K, Y, V, F, A, I, T, L, H, G, Q, R, D, or E, X₈ is selected from Y, F, Q, P, K, T, R, D, I, E, L, H, V, A, G, or S, and X₉ is selected from D, N, A, S, L, K, T, F, I, G, Y, R, V, E, or H, and X₁₀ is selected from S, D, V, H, A, L, E, T, Y, K, F, I, Q, R, G, or P;
      b) amino acid sequences that have at least 80% amino acid identity with the amino acid sequence of X₁X₂X₃X₄X₅X₆X₇X₈X₉X₁₀ (SEQ ID NO: 4610), wherein X₁ is selected from E, P, D, A, K, I, Y, H, R, F, S, T, V, G, or Q, X₂ is selected S, F, A, Y, E, I, D, K, G, R,P, Q, T, H, or L, and X₃ is selected Y, D, F, E, R, K, I, T, A, Q, S, V, H, G, or P, X₄ is selected from G, L, I, H, S, V, R, P, E, A, K, Y, T, F, A, D, H, or R, X₅ is selected from S, D, K, Y, A, L, I, P, V, Q, E, R, T, G, or F, X₆ is selected from S, T, K, P, H, I, G, V, Q, R, D, L, or E, X₇ is selected from W, C, P, K, Y, V, F, A, I, T, L, H, G, Q, R, D, or E, X₈ is selected from Y, F, Q, P, K, T, R, D, I, E, L, H, V, A, G, or S, and X₉ is selected from D, N, A, S, L, K, T, F, I, G, Y, R, V, E, or H, and X₁₀ is selected from S, D, V, H, A, L, E, T, Y, K, F, I, Q, R, G, or P;
      c) amino acid sequences that have 3, 2, or 1 amino acid difference with the amino acid sequence of X₁X₂X₃X₄X₅X₆X₇X₈X₉X₁₀ (SEQ ID NO: 4610), wherein X₁ is selected from E, P, D, A, K, I, Y, H, R, F, S, T, V, G, or Q, X₂ is selected S, F, A, Y, E, I, D, K, G, R,P, Q, T, H, or L, and X₃ is selected Y, D, F, E, R, K, I, T, A, Q, S, V, H, G, or P, X₄ is selected from G, L, I, H, S, V, R, P, E, A, K, Y, T, F, A, D, H, or R, X₅ is selected from S, D, K, Y, A, L, I, P, V, Q, E, R, T, G, or F, X₆ is selected from S, T, K, P, H, I, G, V, Q, R, D, L, or E, X₇ is selected from W, C, P, K, Y, V, F, A, I, T, L, H, G, Q, R, D, or E, X₈ is selected from Y, F, Q, P, K, T, R, D, I, E, L, H, V, A, G, or S, and X₉ is selected from D, N, A, S, L, K, T, F, I, G, Y, R, V, E, or H, and X₁₀ is selected from S, D, V, H, A, L, E, T, Y, K, F, I, Q, R, G, or P.
42. The ISVD according to item 41, in which the amino acid sequences of the CDRs (AbM numbering) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 2857-3662.
43. An immunoglobulin single variable domain (ISVD) specifically binding human PD-1, that essentially consists of 4 framework regions (FR1 to FR4, respectively) and 3 complementarity determining regions (CDR1 to CDR3, respectively), in which:
   CDR1 (AbM numbering) has an amino acid sequence selected from:
      a) the amino acid sequence of GRXHSINAMA (SEQ ID NO: 4704), wherein X is selected from M, V and T;
      b) amino acid sequences that have at least 80% amino acid identity with the amino acid sequence of GRXHSINAMA (SEQ ID NO: 4704), wherein X is selected from M, V and T;
      c) amino acid sequences that have 3, 2, or 1 amino acid difference with the amino acid sequences of GRXHSINAMA (SEQ ID NO: 4704), wherein X is selected from M, V and T;
and
   CDR2 (AbM numbering) has an amino acid sequence selected from:
      a) the amino acid sequence of LISWGXGITY (SEQ ID NO: 4705), wherein X is selected from N and F;
      b) amino acid sequences that have at least 80% amino acid identity with the amino acid sequence of LISWGXGITY (SEQ ID NO: 4705), wherein X is selected from N and F;
      c) amino acid sequences that have 3, 2, or 1 amino acid difference with the amino acid sequence of LISWGXGITY (SEQ ID NO: 4705), wherein X is selected from N and F;
and
   CDR3 (AbM numbering) has an amino acid sequence selected from:
      a) the amino acid sequence of ESYGXSWYDS (SEQ ID NO: 4706); wherein X is selected from S and A;
      b) amino acid sequences that have at least 80% amino acid identity with the amino acid sequence of ESYGXSWYDS (SEQ ID NO: 4706); wherein X is selected from S and A;
      c) amino acid sequences that have 3, 2, or 1 amino acid difference with the amino acid sequence of ESYGXSWYDS (SEQ ID NO: 4706); wherein X is selected from S and A.
44. The ISVD according to any one of items 41 to 43, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of GRMHSINAMA (SEQ ID NO: 2498);
      b) amino acid sequences that have at least 80% amino acid identity with the amino acid sequence of GRMHSINAMA (SEQ ID NO: 2498); and
      c) amino acid sequences that have 3, 2, or 1 amino acid difference with the amino acid sequences of GRMHSINAMA (SEQ ID NO: 2498);
and
   CDR2 (AbM definition) has an amino acid sequence selected from:
      a) the amino acid sequence of LISWGNGITY (SEQ ID NO: 2451);
      b) amino acid sequences that have at least 80% amino acid identity with the amino acid sequence of LISWGNGITY (SEQ ID NO: 2451);
      c) amino acid sequences that have 3, 2, or 1 amino acid difference with the amino acid sequence of LISWGNGITY (SEQ ID NO: 2451);
and
   CDR3 (AbM definition) has an amino acid sequence selected from:
      a) the amino acid sequence of ESYGSSWYDS (SEQ ID NOs: 2804);
      b) amino acid sequences that have at least 80% amino acid identity with the amino acid sequence of ESYGSSWYDS (SEQ ID NOs: 2804);
      c) amino acid sequences that have 3, 2, or 1 amino acid difference with the amino acid sequence of ESYGSSWYDS (SEQ ID NOs: 2804).
45. The ISVD according to any one of items 41 to 43, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of GRVHSINAMA (SEQ ID NO: 2552);
      b) amino acid sequences that have at least 80% amino acid identity with the amino acid sequence of GRVHSINAMA (SEQ ID NO: 2552); and
      c) amino acid sequences that have 3, 2, or 1 amino acid difference with the amino acid sequences of GRVHSINAMA (SEQ ID NO: 2552);
and
   CDR2 (AbM definition) has an amino acid sequence selected from:
      a) the amino acid sequence of LISWGFGITY (SEQ ID NO: 2652);
      b) amino acid sequences that have at least 80% amino acid identity with the amino acid sequence of LISWGFGITY (SEQ ID NO: 2652);
      c) amino acid sequences that have 3, 2, or 1 amino acid difference with the amino acid sequence of LISWGFGITY (SEQ ID NO: 2652);
and
   CDR3 (AbM definition) has an amino acid sequence selected from:
      a) the amino acid sequence of ESYGASWYDS (SEQ ID NOs: 2741);
      b) amino acid sequences that have at least 80% amino acid identity with the amino acid sequence of ESYGASWYDS (SEQ ID NOs: 2741);
      c) amino acid sequences that have 3, 2, or 1 amino acid difference with the amino acid sequence of ESYGASWYDS (SEQ ID NOs: 2741).
46. The ISVD according to any one of items 41 to 43, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of GRTHSINAMA (SEQ ID NO: 2551);
      b) amino acid sequences that have at least 80% amino acid identity with the amino acid sequence of GRTHSINAMA (SEQ ID NO: 2551); and
      c) amino acid sequences that have 3, 2, or 1 amino acid difference with the amino acid sequences of GRTHSINAMA (SEQ ID NO: 2551);
and
   CDR2 (AbM definition) has an amino acid sequence selected from:
      a) the amino acid sequence of LISWGFGITY (SEQ ID NO: 2652);
      b) amino acid sequences that have at least 80% amino acid identity with the amino acid sequence of LISWGFGITY (SEQ ID NO: 2652);
      c) amino acid sequences that have 3, 2, or 1 amino acid difference with the amino acid sequence of LISWGFGITY (SEQ ID NO: 2652);
and
   CDR3 (AbM definition) has an amino acid sequence selected from:
      a) the amino acid sequence of ESYGASWYDS (SEQ ID NO: 2741);
      b) amino acid sequences that have at least 80% amino acid identity with the amino acid sequence of ESYGASWYDS (SEQ ID NOs: 2741);
      c) amino acid sequences that have 3, 2, or 1 amino acid difference with the amino acid sequence of ESYGASWYDS (SEQ ID NOs: 2741).
47. The ISVD according to any one of items 41 to 43, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of GRVHSINAMA (SEQ ID NO: 2552);
      b) amino acid sequences that have at least 80% amino acid identity with the amino acid sequence of GRVHSINAMA (SEQ ID NO: 2552); and
      c) amino acid sequences that have 3, 2, or 1 amino acid difference with the amino acid sequences of GRVHSINAMA (SEQ ID NO: 2552);
and
   CDR2 (AbM definition) has an amino acid sequence selected from:
      a) the amino acid sequence of LISWGFGITY (SEQ ID NO: 2652);
      b) amino acid sequences that have at least 80% amino acid identity with the amino acid sequence of LISWGFGITY (SEQ ID NO: 2652);
      c) amino acid sequences that have 3, 2, or 1 amino acid difference with the amino acid sequence of LISWGFGITY (SEQ ID NO: 2652);
and
   CDR3 (AbM definition) has an amino acid sequence selected from:
      a) the amino acid sequence of ESYGASWYDS (SEQ ID NOs: 2741);
      b) amino acid sequences that have at least 80% amino acid identity with the amino acid sequence of ESYGASWYDS (SEQ ID NO: 2741);
      c) amino acid sequences that have 3, 2, or 1 amino acid difference with the amino acid sequence of ESYGASWYDS (SEQ ID NOs: 2741).
48. The ISVD according to any one of items 41 to 43, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of GRTHSINAMA (SEQ ID NO: 2551);
      b) amino acid sequences that have at least 80% amino acid identity with the amino acid sequence of GRTHSINAMA (SEQ ID NO: 2551); and
      c) amino acid sequences that have 3, 2, or 1 amino acid difference with the amino acid sequences of GRTHSINAMA (SEQ ID NO: 2551);
and
   CDR2 (AbM definition) has an amino acid sequence selected from:
      a) the amino acid sequence of LISWGFGITY (SEQ ID NO: 2652);
      b) amino acid sequences that have at least 80% amino acid identity with the amino acid sequence of LISWGFGITY (SEQ ID NO: 2652);
      c) amino acid sequences that have 3, 2, or 1 amino acid difference with the amino acid sequence of LISWGFGITY (SEQ ID NO: 2652);
and
   CDR3 (AbM definition) has an amino acid sequence selected from:
      a) the amino acid sequence of ESYGSSWYDS (SEQ ID NOs: 2804);
      b) amino acid sequences that have at least 80% amino acid identity with the amino acid sequence of ESYGSSWYDS (SEQ ID NOs: 2804);
      c) amino acid sequences that have 3, 2, or 1 amino acid difference with the amino acid sequence of ESYGSSWYDS (SEQ ID NOs: 2804).
49. The ISVD according to any one of items 41 to 48, in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2453, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2456, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2559, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 3810, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2455, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2556, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2560, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2557, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2450, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2561, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2454, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2558, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2458, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2460, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2553, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2459, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2554, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2464, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2466, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2462, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2465, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2457, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2463, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2461, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2470, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2549, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2550, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2471, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2468, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2552, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2472, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2551, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2555, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2467, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2469, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2473, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2542, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2547, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2476, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2543, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2480, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2544, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2540, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2477, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2475, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2548, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2545, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2538, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2474, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2546, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2541, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2535, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2486, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2482, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2484, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2537, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2483, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2481, CDR2 (AbM numbering) consists of the amino acid sequence of SEQ ID NO: 2451, CDR3 (AbM numbering) consists of the amino acid sequence of SEQ ID NO: 2815; or in which
   CDR1 (AbM numbering) consists of the amino acid sequence of SEQ ID NO: 2536, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2478, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2479, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2539, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2481, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2487, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2485, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2534, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2528, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2490, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2489, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2529, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2530, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2531, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2492, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2533, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2532, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2488, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2494, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2525, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2491, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2495, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2526, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2524, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2527, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2493, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2521, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2496, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2497, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2512, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2511, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2516, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2515, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2520, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2605, CDR3 (AbM numbering) consists of the amino acid sequence of SEQ ID NO: 2804; or in which
   CDR1 (AbM numbering) consists of the amino acid sequence of SEQ ID NO: 2522, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2517, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2518, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2514, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2519, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2523, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2499, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2502, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2503, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2504, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2500, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2505, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2509, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2508, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2510, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2501, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2513, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2507, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2506, CDR2 (AbM numbering) consists of the amino acid sequence of SEQ ID NO: 2451, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2698, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2569, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2699, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2564, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2562, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2705, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2695, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2697, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2566, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2700, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2563, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2706, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2565, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2570, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2572, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2701, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2571, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2619, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2575, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2704, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2694, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2702, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2568, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2703, CDR3 (AbM numbering) consists of the amino acid sequence of SEQ ID NO: 2804; or in which
   CDR1 (AbM numbering) consists of the amino acid sequence of SEQ ID NO: 2533, CDR2 (AbM numbering) consists of the amino acid sequence of SEQ ID NO: 2674, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2596, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2577, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2587, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2583, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2693, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2580, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2579, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2581, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2688, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2578, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2574, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2573, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2582, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2585, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2586, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2593, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2598, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2589, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2588, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2689, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2594, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2600, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2590, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2609, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2591, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2601, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2597, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2595, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2592, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2603, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2602, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2681, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2686, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2690, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2685, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2687, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2683, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2682, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2604, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2599, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2684, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2610, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2678, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2606, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2652, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2651, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2677, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2679, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2672, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2613, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2611, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2676, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2680, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2669, CDR3 (AbM numbering) consists of the amino acid sequence of SEQ ID NO: 2804; or in which
   CDR1 (AbM numbering) consists of the amino acid sequence of SEQ ID NO: 2509, CDR2 (AbM numbering) consists of the amino acid sequence of SEQ ID NO: 2612, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2614, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2671, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2663, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2664, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2668, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2615, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2673, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2670, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2667, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2616, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2675, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2617, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2661, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2659, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2658, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2657, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2666, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2665, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2625, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2654, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2662, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2622, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2618, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2620, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2621, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2660, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2656, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2634, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2626, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2631, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2632, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2633, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2627, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2628, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2623, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2624, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2655, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2630, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2653, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2629, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2642, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2643, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2647, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2636, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2635, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2637, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2648, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2644, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2638, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2639, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2645, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2646, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2641, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2640, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2649, CDR3 (AbM numbering) consists of the amino acid sequence of SEQ ID NO: 2804; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2742; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2850; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2707; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2708; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2849; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2848; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2856; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2847; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2852; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2845; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2853; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2854; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2855; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2846; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2851; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2710; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2709; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2844; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2711; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2715; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2712; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2716; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2714; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2718; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2720; or in which
   CDR1 (AbM numbering) consists of the amino acid sequence of SEQ ID NO: 2498, CDR2 (AbM numbering) consists of the amino acid sequence of SEQ ID NO: 2584, CDR3 (AbM numbering) consists of the amino acid sequence of SEQ ID NO: 2844; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2719; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2843; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2713; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2717; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2722; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2725; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2723; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2732; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2728; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2726; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2731; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2721; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2729; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2730; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2734; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2727; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2724; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2733; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2833; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2831; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2830; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2839; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2841; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2838; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2834; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2738; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2735; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2832; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2842; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2840; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2739; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2736; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2737; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2771; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2769; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2821; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2775; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2765; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2762; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2768; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2774; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2770; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2767; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2766; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2772; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2773; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2763; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2764; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2779; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2786; or in which
   CDR1 (AbM numbering) consists of the amino acid sequence of SEQ ID NO: 2498, CDR2 (AbM numbering) consists of the amino acid sequence of SEQ ID NO: 2671, CDR3 (AbM numbering) consists of the amino acid sequence of SEQ ID NO: 2785; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2783; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2789; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2787; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2777; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2782; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2778; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2784; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2785; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2781; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2790; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2776; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2780; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2788; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2791; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2817; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2814; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2813; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2818; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2809; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2812; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2810; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2820; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2816; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2819; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2808; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2811; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2793; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2806; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2797; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2792; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2800; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2794; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2805; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2807; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2799; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2795; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2798; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2802; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2803; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2796; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2801; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2747; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2826; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2741; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2748; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2746; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2749; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2825; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2750; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2743; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2751; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2824; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2745; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2744; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2822; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2757; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2759; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2755; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2756; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2754; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2823; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2760; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2761; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2752; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2758; or in which
   CDR1 (AbM numbering) consists of the amino acid sequence of SEQ ID NO: 2498, CDR2 (AbM numbering) consists of the amino acid sequence of SEQ ID NO: 2451, CDR3 (AbM numbering) consists of the amino acid sequence of SEQ ID NO: 2753; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2804; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2804; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2804; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2804; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2830; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2830; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2830; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2830; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2838; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2838; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2838; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2838; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2741; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2741; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2741; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2741; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2828; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2828; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2828; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2828; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2836; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2836; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2836; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2836; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2785; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2785; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2785; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2785; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2829; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2829; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2829; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2829; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2837; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2837; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2837; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2837; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2740; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2740; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2740; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2740; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2827; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2827; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2827; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2827; or in which
   CDR1 (AbM numbering) consists of the amino acid sequence of SEQ ID NO: 2551, CDR2 (AbM numbering) consists of the amino acid sequence of SEQ ID NO: 2651, CDR3 (AbM numbering) consists of the amino acid sequence of SEQ ID NO: 2835; or in which
   CDR1 (AbM numbering) consists of the amino acid sequence of SEQ ID NO: 2552, CDR2 (AbM numbering) consists of the amino acid sequence of SEQ ID NO: 2651, CDR3 (AbM numbering) consists of the amino acid sequence of SEQ ID NO: 2835; or in which
   CDR1 (AbM numbering) consists of the amino acid sequence of SEQ ID NO: 2551, CDR2 (AbM numbering) consists of the amino acid sequence of SEQ ID NO: 2652, CDR3 (AbM numbering) consists of the amino acid sequence of SEQ ID NO: 2835; or in which
   CDR1 (AbM numbering) consists of the amino acid sequence of SEQ ID NO: 2552, CDR2 (AbM numbering) consists of the amino acid sequence of SEQ ID NO: 2652, CDR3 (AbM numbering) consists of the amino acid sequence of SEQ ID NO: 2835; or in which
   CDR1 (AbM numbering) consists of the amino acid sequence of SEQ ID NO: 2551, CDR2 (AbM numbering) consists of the amino acid sequence of SEQ ID NO: 2608, CDR3 (AbM numbering) consists of the amino acid sequence of SEQ ID NO: 2804; or in which
   CDR1 (AbM numbering) consists of the amino acid sequence of SEQ ID NO: 2551, CDR2 (AbM numbering) consists of the amino acid sequence of SEQ ID NO: 2576, CDR3 (AbM numbering) consists of the amino acid sequence of SEQ ID NO: 2804; or in which
   CDR1 (AbM numbering) consists of the amino acid sequence of SEQ ID NO: 2551, CDR2 (AbM numbering) consists of the amino acid sequence of SEQ ID NO: 2691, CDR3 (AbM numbering) consists of the amino acid sequence of SEQ ID NO: 2804; or in which
   CDR1 (AbM numbering) consists of the amino acid sequence of SEQ ID NO: 2552, CDR2 (AbM numbering) consists of the amino acid sequence of SEQ ID NO: 2691, CDR3 (AbM numbering) consists of the amino acid sequence of SEQ ID NO: 2741; or in which
   CDR1 (AbM numbering) consists of the amino acid sequence of SEQ ID NO: 2552, CDR2 (AbM numbering) consists of the amino acid sequence of SEQ ID NO: 2567, CDR3 (AbM numbering) consists of the amino acid sequence of SEQ ID NO: 2741; or in which
   CDR1 (AbM numbering) consists of the amino acid sequence of SEQ ID NO: 2552, CDR2 (AbM numbering) consists of the amino acid sequence of SEQ ID NO: 2576, CDR3 (AbM numbering) consists of the amino acid sequence of SEQ ID NO: 2741; or in which
   CDR1 (AbM numbering) consists of the amino acid sequence of SEQ ID NO: 2552, CDR2 (AbM numbering) consists of the amino acid sequence of SEQ ID NO: 2696, CDR3 (AbM numbering) consists of the amino acid sequence of SEQ ID NO: 2741; or in which
   CDR1 (AbM numbering) consists of the amino acid sequence of SEQ ID NO: 2551, CDR2 (AbM numbering) consists of the amino acid sequence of SEQ ID NO: 2567, CDR3 (AbM numbering) consists of the amino acid sequence of SEQ ID NO: 2804; or in which
   CDR1 (AbM numbering) consists of the amino acid sequence of SEQ ID NO: 2551, CDR2 (AbM numbering) consists of the amino acid sequence of SEQ ID NO: 2696, CDR3 (AbM numbering) consists of the amino acid sequence of SEQ ID NO: 2804; or in which
   CDR1 (AbM numbering) consists of the amino acid sequence of SEQ ID NO: 2552, CDR2 (AbM numbering) consists of the amino acid sequence of SEQ ID NO: 2678, CDR3 (AbM numbering) consists of the amino acid sequence of SEQ ID NO: 2741; or in which
   CDR1 (AbM numbering) consists of the amino acid sequence of SEQ ID NO: 2552, CDR2 (AbM numbering) consists of the amino acid sequence of SEQ ID NO: 2677, CDR3 (AbM numbering) consists of the amino acid sequence of SEQ ID NO: 2741; or in which
   CDR1 (AbM numbering) consists of the amino acid sequence of SEQ ID NO: 2551, CDR2 (AbM numbering) consists of the amino acid sequence of SEQ ID NO: 2678, CDR3 (AbM numbering) consists of the amino acid sequence of SEQ ID NO: 2741; or in which
   CDR1 (AbM numbering) consists of the amino acid sequence of SEQ ID NO: 2551, CDR2 (AbM numbering) consists of the amino acid sequence of SEQ ID NO: 2677, CDR3 (AbM numbering) consists of the amino acid sequence of SEQ ID NO: 2741; or in which
   CDR1 (AbM numbering) consists of the amino acid sequence of SEQ ID NO: 2551, CDR2 (AbM numbering) consists of the amino acid sequence of SEQ ID NO: 2678, CDR3 (AbM numbering) consists of the amino acid sequence of SEQ ID NO: 2804; or in which
   CDR1 (AbM numbering) consists of the amino acid sequence of SEQ ID NO: 2551, CDR2 (AbM numbering) consists of the amino acid sequence of SEQ ID NO: 2677, CDR3 (AbM numbering) consists of the amino acid sequence of SEQ ID NO: 2804.
50. The ISVD according to any one of items 41 to 49, wherein the ISVD further comprises R45 (Kabat numbering).
51. The ISVD according to any one of items 41 to 50, wherein the ISVD further comprises F37 and/or F47 (Kabat numbering).
52. The ISVD according to any of one items 41 to 51, which amino acid sequence
   i) has 80% amino acid sequence identity with one of the amino acid sequences of SEQ ID NOs: 2857-3662, in which for the purposes of determining the degree of amino acid identity, the amino acid residues that form the CDR sequences are disregarded;
   and in which:
   ii) preferably one or more of the amino acid residues at positions 11, 37, 44, 45, 47, 83, 84, 103, 104 and 108 according to the Kabat numbering are chosen from the Hallmark residues mentioned in Table 1.
53. The ISVD according to any one of items 1 to 52, that essentially consists of a heavy chain variable domain sequence that is derived from a conventional four-chain antibody or that essentially consist of a heavy chain variable domain sequence that is derived from heavy chain antibody.
54. The ISVD according to any one of items 1 to 53, that essentially consists of a VHH, a humanized VHH, a camelized VH, a domain antibody, a single domain antibody, or a dAb.
55. The ISVD according to any one of items 1 to 54, which is a humanized ISVD that is chosen from SEQ ID NOs: 2857-3394 and 3396-3662 or from the group consisting of amino acid sequences that have more than 80%, preferably more than 90%, more preferably more than 95%, such as 99% or more amino acid sequence identity with at least one of the amino acid sequences of SEQ ID NOs: 2857-3662.
56. The ISVD according to any one of items 1 to 55, in which the amino acid sequence is chosen from the group consisting of SEQ ID NO: 3395 and SEQ ID NOs: 2857-3394, 3396-3662.
57. The ISVD according to any one of items 1 to 56, wherein the ISVD specifically binds to human PD-1 with a dissociation constant (K_{D}) of 5×10⁻⁹ to 10⁻¹² moles/litre or less, and preferably 10⁻⁹ to 10⁻¹² moles/litre or less and more preferably 10⁻⁹ to 5×10⁻¹¹ moles/litre, such as 7.5×10⁻¹⁰ moles/litre, as determined by Surface Plasmon Resonance.
58. The ISVD according to any one of items 1 to 57, wherein the ISVD specifically binds to human PD-1 with a kₒₙ-rate of between 10⁴ M⁻¹ s⁻¹ to about 10⁷ M⁻¹ s⁻¹, preferably between 5×10⁴ M⁻¹ s⁻¹ and 10⁷ M⁻¹ s⁻¹, more preferably between 5×10⁴ M⁻¹ s⁻¹ and 5×10⁶ M⁻¹ s⁻¹, such as between 5×10⁴ M⁻¹ s⁻¹ and 1×10⁶ M⁻¹ s⁻¹, such as 8.8×10⁴ M⁻¹ s⁻¹, as determined by Surface Plasmon Resonance.
59. The ISVD according to any one of items 1 to 58, wherein the ISVD specifically binds to human PD-1 with a k_{off} rate between 10³ s⁻¹ (t1/2=0.69 s) and 10⁻⁸ s⁻¹ (providing a near irreversible complex with a t1/2 of multiple days), preferably between 10⁻³ s⁻¹ and 5×10⁻⁷ s⁻¹, more preferably between 5×10⁻⁴ s⁻¹ and 5×10⁻⁶ s⁻¹, such as between 10⁻⁴ s⁻¹ and 10⁻⁵ s⁻¹, such as 6.6×10⁵ s⁻¹, as determined by Surface Plasmon Resonance.
60. The ISVD according to any one of items 1 to 59, wherein the ISVD specifically binds to human and cyno PD-1 and does not bind to CD28, CTLA-4, ICOS, and BTLA or other members of the CD28 receptor family.
61. The ISVD according to any one of items 1 to 60, wherein the ISVD antagonizes an activity of PD-L1.
62. The ISVD according to any one of items 1 to 61, wherein the ISVD blocks the interaction of human PD-1 with human PD-L1 with a potency (EC50 value) of 10⁻⁸ M or lower, more preferably of 5×10⁻⁹ M or lower, or even of 10⁹ M or lower, such as between 10⁻¹¹ M and 10⁻⁸ M, between 10⁻¹⁰ M and 5×10⁻⁹ M, between 10⁻¹⁰ M and 10⁻⁸ M or between 10⁻⁹ M and 10⁻⁸ M, such as 5.94×10⁻⁹ M, for example, as measured in a FACS competition assay on CHO cells expressing human PD-1 with hPD-L1 as a competitor.
63. The ISVD according to any one of items 1 to 62, wherein the ISVD blocks the interaction of cyno PD-1 with cyno PD-L1 with a potency (EC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 5×10⁻⁹ M, between 10⁻¹⁰ M and 10⁻⁸ M or between 10⁻⁹ M and 10⁻⁸ M, such as 4.7×10⁻⁹ M, for example, as measured in a FACS competition assay on CHO cells expressing cyno PD-1 with hPD-L1 as a competitor.
64. The ISVD according to any one of items 1 to 63, wherein the ISVD blocks the binding of hPD-1 to hPD-L1 by at least 50%, such as at least 60%, 70%, 80%, 90%, 95%, 99% or even more, as determined by ligand competition, AlphaScreen, or competitive binding assays (such as competition ELISA or competition FACS).
65. The ISVD according to any one of items 1 to 64, wherein the ISVD induces T-cell activation with a potency (EC50 value) of 10⁻⁷ M or lower, more preferably of 10⁻⁸ M or lower, or even of 5·10⁻⁹ M or lower, such as between 10⁻¹¹ M and 10⁻⁷ M, between 10⁻¹⁰ M and 5×10⁻⁸ M, between 10⁻¹⁰ M and 10⁻⁸ M or between 10⁻¹¹ M and 10⁻⁹ M, such as 2.69×10⁻⁸ M, or 3.37×10⁻⁸ M, as determined in a TT assay.
66. A polypeptide or construct that comprises or essentially consists of one or more ISVDs according to any one of items 1 to 65, and optionally further comprises one or more other groups, residues, moieties or binding units, optionally linked via one or more linkers.
67. The polypeptide or construct according to item 66, in which said one or more other groups, residues, moieties or binding units are amino acid sequences.
68. The polypeptide or construct according to any one of items 66 to 67, in which said one or more linkers are one or more amino acid sequences.
69. The polypeptide or construct according to any one of items 66 to 68, in which said one or more other groups, residues, moieties or binding units are immunoglobulin sequences.
70. The polypeptide or construct according to any one of items 66 to 69, in which said one or more other groups, residues, moieties or binding units are ISVDs.
71. The polypeptide or construct according to any one of items 66 to 70, in which said one or more other groups, residues, moieties or binding units are chosen from the group consisting of VHHs, humanized VHHs, camelized VHs, domain antibodies, single domain antibodies and dAbs.
72. The polypeptide or construct according to any one of items 66 to 71, which is a multivalent construct.
73. The polypeptide or construct according to any one of items 66 to 72, which is a multispecific construct.
74. The polypeptide or construct according to any one of items 66 to 73, in which said one or more other groups, residues, moieties or binding units provide the polypeptide or construct with increased half-life, compared to the ISVD without the one or more other groups, residues, moieties or binding units.
75. The polypeptide or construct according to item 74, in which said one or more other groups, residues, moieties or binding units that provide the polypeptide or construct with increased half-life is chosen from the group consisting of a polyethylene glycol molecule (PEG), serum proteins or fragments thereof, binding units that specifically bind to serum proteins, an Fc portion, and small proteins or peptides that specifically bind to serum proteins.
76. The polypeptide or construct according to item 75, in which said one or more other groups, residues, moieties or binding units that provide the polypeptide or construct with increased half-life is chosen from the group consisting of human serum albumin or fragments thereof.
77. The polypeptide or construct according to item 75, in which said one or more other groups, residues, moieties or binding units that provides the polypeptide or construct with increased half-life are chosen from the group consisting of binding units that specifically bind to serum albumin (such as human serum albumin) or a serum immunoglobulin (such as IgG).
78. The polypeptide or construct according to item 77, in which said one or more other groups, residues, moieties or binding units that provides the polypeptide or construct with increased half-life are chosen from the group consisting of VHHs, humanized VHHs, camelized VHs, domain antibodies, single domain antibodies, or dAbs that specifically bind to serum albumin (such as human serum albumin) or a serum immunoglobulin (such as IgG).
79. The polypeptide or construct according to item 78, in which said one or more other groups, residues, moieties or binding units that provides the polypeptide or construct with increased half-life is an ISVD that specifically binds human serum albumin.
80. The polypeptide or construct according to item 79, wherein said ISVD that specifically binds human serum albumin essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of SEQ ID NO: 2436;
      b) amino acid sequences that have at least 80% amino acid identity with the amino acid sequence of SEQ ID NO: 2436; and
      c) amino acid sequences that have 3, 2, or 1 amino acid difference with the amino acid sequences of SEQ ID NO: 2436;
and
   CDR2 (AbM definition) has an amino acid sequence selected from:
      a) the amino acid sequence of SEQ ID NO: 2438;
      b) amino acid sequences that have at least 80% amino acid identity with the amino acid sequence of SEQ ID NO: 2438;
      c) amino acid sequences that have 3, 2, or 1 amino acid difference with the amino acid sequence of SEQ ID NO: 2438;
and
   CDR3 (AbM definition) has an amino acid sequence selected from:
      a) the amino acid sequence of SEQ ID NO: 2440;
      b) amino acid sequences that have at least 80% amino acid identity with the amino acid sequence of SEQ ID NO: 2440;
      c) amino acid sequences that have 3, 2, or 1 amino acid difference with the amino acid sequence of SEQ ID NO: 2440.
81. The polypeptide or construct according to item 80, wherein CDR1 consists of the amino acid sequence of SEQ ID NO: 2436, CDR2 consists of the amino acid sequence of SEQ ID NO: 2438, and CDR3 consists of the amino acid sequence of SEQ ID NO: 2440.
82. The polypeptide or construct according to any one of items 79 to 81, wherein said ISVD that specifically binds human serum albumin is selected from the group consisting of ALB8 (SEQ ID NO: 2401), ALB23 (SEQ ID NO: 2402), ALBX00001 (SEQ ID NO: 2415) and ALB23002 (SEQ ID NO: 2416), preferably the ISVD binding to human serum albumin is ALB23002 (SEQ ID NO: 2416).
83. The polypeptide or construct according to anyone of items 66 to 82, wherein said linker is chosen from the group consisting of SEQ ID NOs: 2418 to 2434.
84. The polypeptide or construct according to any of items 66 to 83, further comprising a C-terminal extension.
85. The polypeptide or construct according to item 84, wherein said C-terminal extension is a C-terminal extension (X)ₙ, in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I).
86. A nucleic acid that encodes an ISVD according to any one of items 1 to 65, or a polypeptide according to any one of items 66 to 85.
87. The nucleic acid according to item 86, that is in the form of a genetic construct.
88. A non-human host or host cell that expresses, or that under suitable circumstances is capable of expressing, an ISVD according to any of one items 1 to 65, or a polypeptide according to any one of items 66 to 85; and/or that comprises the nucleic acid according to any one of items 86 or 87.
89. A method for producing an ISVD according to any one of items 1 to 65, or a polypeptide according to any one of items 66 to 85, at least comprising the steps of:
   a) expressing, in a suitable (non-human) host organism or in a host cell or in another suitable expression system, a nucleic acid according to any one of items 86 or 87;
optionally followed by:
   b) isolating and/or purifying the ISVD according to any one of items 1 to 65, or the polypeptide according to any one of items 66 to 85.
90. A method for producing an ISVD according to any one of items 1 to 65, or a polypeptide according to any one of items 66 to 85, said method at least comprising the steps of:
   a) cultivating and/or maintaining a (non-human) host or host cell according to item 88 under conditions that are such that said non-human host or host cell expresses and/or produces at least one ISVD according to any one of items 1 to 65, or at least one polypeptide according to any one of items 66 to 85;
optionally followed by:
   b) isolating and/or purifying the ISVD according to any one of items 1 to 65, or polypeptide according to any one of items 66 to 85.
91. A composition comprising at least one ISVD according to any one of items 1 to 65, at least one polypeptide or construct according to any one of items 66 to 85, or at least one nucleic acid according to any one of items 66 or 87.
92. The composition according to item 91, which is a pharmaceutical composition.
93. The composition according to any one of items 91 or 92, which is a pharmaceutical composition, that further comprises at least one pharmaceutically acceptable carrier, diluent or excipient and/or adjuvant, and that optionally comprises one or more further pharmaceutically active polypeptides and/or compounds.
94. The ISVD according to any one of items 1 to 65, the polypeptide or construct according to any one of items 66 to 85, or the composition according to any one of items 91 to 93, for use as a medicament.
95. The ISVD according to any one of items 1 to 65, the polypeptide or construct according to any one of items 66 to 85, or the composition according to any one of items 91 to 93, for use in the diagnosis, prevention and/or treatment of at least one disease and/or disorder.
96. The ISVD according to any one of items 1 to 65, the polypeptide or construct according to any one of items 66 to 85, or the composition according to any one of items 91 to 93, for use in the diagnosis, prevention and/or treatment of at least one disease and/or disorder that is associated with PD-1, with its biological or pharmacological activity, and/or with the biological pathways or signalling in which PD-1 is involved.
97. The ISVD according to any one of items 1 to 65, the polypeptide or construct according to any one of items 66 to 85, or the composition according to any one of items 91 to 93, for use in the diagnosis, prevention and/or treatment of cancer.
98. A method for the diagnosis, prevention and/or treatment of at least one disease and/or disorder, comprising the administration, to a subject in need thereof, of a pharmaceutically active amount of an ISVD according to any one of items 1 to 65, a polypeptide or construct according to any one of items 66 to 85, or a composition according to any one of items 91 to 93.
99. The method according to item 98, for the diagnosis, prevention and/or treatment of at least one disease or disorder that is associated with PD-1, with its biological or pharmacological activity, and/or with the biological pathways or signalling in which PD-1 is involved, said method comprising administering, to a subject in need thereof, a pharmaceutically active amount of at least one ISVD according to any one of items 1 to 65, a polypeptide or construct according to any one of items 66 to 85, or composition according to any one of items 91 to 93.
100. The method according to any one of items 98 or 99, for the diagnosis, prevention and/or treatment of cancer, said method comprising administering, to a subject in need thereof, a pharmaceutically active amount of at least one ISVD according to any one of items 1 to 65, a polypeptide or construct according to any one of items 66 to 85, or a composition according to any one of items 91 to 93.

### 7.4 Embodiment 4, Family A0294040C12

1. An immunoglobulin single variable domain (ISVD) specifically binding human PD-1, that essentially consists of 4 framework regions (FR1 to FR4, respectively) and 3 complementarity determining regions (CDR1 to CDR3, respectively), in which:
   CDR1 (AbM definition) has an amino acid sequence selected from:
   a) the amino acid sequence of GFTLX₁X₂X₃X₄X₅X₆ (SEQ ID NO: 4696), wherein X₁ is selected from Y, F, T, Q, A, S, R, P, D, G, L, E, K, H, or D, X₂ is selected from D, R, T, I, Q, F, H, S, L, V, K, E, P, G, A, or Y, X₃ is selected from H, G, Y, R, D, E, V, P, Q, S, A, K, I, F, T, or L, X₄ is selected from A, L, S, D, R, K, H, Q, T, V, Y, P, I, F, or G, X₅ is selected from V and F, and X₆ is selected from G, Q, E, H, K, P, Y, D, I, T, A, S, R, V, or C;
   b) amino acid sequences that have at least 80% amino acid identity with the amino acid sequence of GFTLX₁X₂X₃X₄X₅X₆ (SEQ ID NO: 4696), wherein X₁ is selected from Y, F, T, Q, A, S, R, P, D, G, L, E, K, H, or D, X₂ is selected from D, R, T, I, Q, F, H, S, L, V, K, E, P, G, A, or Y, X₃ is selected from H, G, Y, R, D, E, V, P, Q, S, A, K, I, F, T, or L, X₄ is selected from A, L, S, D, R, K, H, Q, T, V, Y, P, I, F, or G, X₅ is selected from V and F, and X₆ is selected from G, Q, E, H, K, P, Y, D, I, T, A, S, R, V, or C;
   c) amino acid sequences that have 3, 2, or 1 amino acid difference with the amino acid sequences of GFTLX₁X₂X₃X₄X₅X₆ (SEQ ID NO: 4696), wherein X₁ is selected from Y, F, T, Q, A, S, R, P, D, G, L, E, K, H, or D, X₂ is selected from D, R, T, I, Q, F, H, S, L, V, K, E, P, G, A, or Y, X₃ is selected from H, G, Y, R, D, E, V, P, Q, S, A, K, I, F, T, or L, X₄ is selected from A, L, S, D, R, K, H, Q, T, V, Y, P, I, F, or G, X₅ is selected from V and F, and X₆ is selected from G, Q, E, H, K, P, Y, D, I, T, A, S, R, V, or C;
and
   CDR2 (AbM definition) has an amino acid sequence selected from:
   a) the amino acid sequence of X₁X₂SSSGX₃ HEX₄ (SEQ ID NO: 4697), wherein X₁ is selected from V, E, or D, X₂ is selected from I, P, T, S, R, G, H, or K, X₃ is selected from A, R, H, E, K, T, or P, and X₄ is selected from W, F, E, P, D, H, Y, L, R, V, K, G, S, A, or l;
   b) amino acid sequences that have at least 80% amino acid identity with the amino acid sequence of X₁X₂SSSGX₃HEX₄ (SEQ ID NO: 4697), wherein X₁ is selected from V, E, or D, X₂ is selected from I, P, T, S, R, G, H, or K, X₃ is selected from A, R, H, E, K, T, or P, and X₄ is selected from W, F, E, P, D, H, Y, L, R, V, K, G, S, A, or l;
   c) amino acid sequences that have 3, 2, or 1 amino acid difference with the amino acid sequence of X₁X₂SSSGX₃ HEX₄ (SEQ ID NO: 4697), wherein X₁ is selected from V, E, or D, X₂ is selected from I, P, T, S, R, G, H, or K, X₃ is selected from A, R, H, E, K, T, or P, and X₄ is selected from W, F, E, P, D, H, Y, L, R, V, K, G, S, A, or l;
and
   CDR3 (AbM definition) has an amino acid sequence selected from:
   a) the amino acid sequence of GX₁X₂X₃YSX₄X₅X₆SEY (SEQ ID NO: 4698); wherein X₁ is selected from G or R, X₂ is selected from M, E, Q, F, V, G, A, I, D, Y, R, K, S, T, H, or P, X₃ is selected from W, L, T, S, P, F, V, I, R, L, Q, E, K, G, D, Y, H, or A, X₄ is selected from D, G, H, K, P, R, or A, X₅ is selected from D, A, V, S, E, I, H, Y, L, G, P, K, Q, R, T, or F, and X₆ is selected from G, F, S, H, Y, I, R, K, L, T, P, V, D, E, Q, or A;
   b) amino acid sequences that have at least 80% amino acid identity with the amino acid sequence of GX₁X₂X₃YSX₄X₅X₆SEY (SEQ ID NO: 4698); wherein X₁ is selected from G or R, X₂ is selected from M, E, Q, F, V, G, A, I, D, Y, R, K, S, T, H, or P, X₃ is selected from W, L, T, S, P, F, V, I, R, L, Q, E, K, G, D, Y, H, or A, X₄ is selected from D, G, H, K, P, R, or A, X₅ is selected from D, A, V, S, E, I, H, Y, L, G, P, K, Q, R, T, or F, and X₆ is selected from G, F, S, H, Y, I, R, K, L, T, P, V, D, E, Q, or A;
   c) amino acid sequences that have 3, 2, or 1 amino acid difference with the amino acid sequence of GX₁X₂X₃YSX₄X₅X₆SEY (SEQ ID NO: 4698); wherein X₁ is selected from G or R, X₂ is selected from M, E, Q, F, V, G, A, I, D, Y, R, K, S, T, H, or P, X₃ is selected from W, L, T, S, P, F, V, I, R, L, Q, E, K, G, D, Y, H, or A, X₄ is selected from D, G, H, K, P, R, or A, X₅ is selected from D, A, V, S, E, I, H, Y, L, G, P, K, Q, R, T, or F, and X₆ is selected from G, F, S, H, Y, I, R, K, L, T, P, V, D, E, Q, or A.
2. The ISVD according to item 1, in which the amino acid sequences of the CDRs (AbM definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 4049-4378.
3. An immunoglobulin single variable domain (ISVD) specifically binding human PD-1, that essentially consists of 4 framework regions (FR1 to FR4, respectively) and 3 complementarity determining regions (CDR1 to CDR3, respectively), in which:
   CDR1 (AbM numbering) has an amino acid sequence selected from:
   a) the amino acid sequence of GFTLYDHAXG (SEQ ID NO: 4611), wherein X is selected from V or F;
   b) amino acid sequences that have at least 80% amino acid identity with the amino acid sequence of GFTLYDHAXG (SEQ ID NO: 4611), wherein X is selected from V or F;
   c) amino acid sequences that have 3, 2, or 1 amino acid difference with the amino acid sequences of GFTLYDHAXG (SEQ ID NO: 4611), wherein X is selected from V or F;
and
   CDR2 (AbM numbering) has an amino acid sequence selected from:
   a) the amino acid sequence of VISSSGAHEW (SEQ ID NO: 4481);
   b) amino acid sequences that have at least 80% amino acid identity with the amino acid sequence of VISSSGAHEW (SEQ ID NO: 4481);
   c) amino acid sequences that have 3, 2, or 1 amino acid difference with the amino acid sequence of VISSSGAHEW (SEQ ID NO: 4481);
and
   CDR3 (AbM numbering) has an amino acid sequence selected from:
   a) the amino acid sequence of GGMWYSDXGSEY (SEQ ID NO: 4612); wherein X is selected from D or E;
   b) amino acid sequences that have at least 80% amino acid identity with the amino acid sequence of GGMWYSDXGSEY (SEQ ID NO: 4612); wherein X is selected from D or E;
   c) amino acid sequences that have 3, 2, or 1 amino acid difference with the amino acid sequence of GGMWYSDXGSEY (SEQ ID NO: 4612); wherein X is selected from D or E.
4. The ISVD according to any one of item 1 or 2, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of GFTLYDHAVG (SEQ ID NO: 4412);
      b) amino acid sequences that have at least 80% amino acid identity with the amino acid sequence of SEQ ID NO: GFTLYDHAVG (SEQ ID NO: 4412); and
      c) amino acid sequences that have 3, 2, or 1 amino acid difference with the amino acid sequences of SEQ ID NO: GFTLYDHAVG (SEQ ID NO: 4412);
and
   CDR2 (AbM definition) has an amino acid sequence selected from:
      a) the amino acid sequence of VISSSGAHEW (SEQ ID NO: 4481);
      b) amino acid sequences that have at least 80% amino acid identity with the amino acid sequence VISSSGAHEW (SEQ ID NO: 4481);
      c) amino acid sequences that have 3, 2, or 1 amino acid difference with the amino acid sequence of VISSSGAHEW (SEQ ID NO: 4481);
and
   CDR3 (AbM definition) has an amino acid sequence selected from:
      a) the amino acid sequence of GGMWYSDDGSEY (SEQ ID NO: 4560);
      b) amino acid sequences that have at least 80% amino acid identity with the amino acid sequence of SEQ ID NO: GGMWYSDDGSEY (SEQ ID NO: 4560);
      c) amino acid sequences that have 3, 2, or 1 amino acid difference with the amino acid sequence of SEQ ID NO: GGMWYSDDGSEY (SEQ ID NO: 4560).
5. The ISVD according to any one of item 1 or 2, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of GFTLYDHAVG (SEQ ID NO: 4412);
      b) amino acid sequences that have at least 80% amino acid identity with the amino acid sequence of SEQ ID NO: GFTLYDHAVG (SEQ ID NO: 4412); and
      c) amino acid sequences that have 3, 2, or 1 amino acid difference with the amino acid sequences of SEQ ID NO: GFTLYDHAVG (SEQ ID NO: 4412);
and
   CDR2 (AbM definition) has an amino acid sequence selected from:
      a) the amino acid sequence of VISSSGAHEW (SEQ ID NO: 4481);
      b) amino acid sequences that have at least 80% amino acid identity with the amino acid sequence VISSSGAHEW (SEQ ID NO: 4481);
      c) amino acid sequences that have 3, 2, or 1 amino acid difference with the amino acid sequence of VISSSGAHEW (SEQ ID NO: 4481);
and
   CDR3 (AbM definition) has an amino acid sequence selected from:
      a) the amino acid sequence of GGMWYSDEGSEY (SEQ ID NO: 4572);
      b) amino acid sequences that have at least 80% amino acid identity with the amino acid sequence of SEQ ID NO: GGMWYSDEGSEY (SEQ ID NO: 4572);
      c) amino acid sequences that have 3, 2, or 1 amino acid difference with the amino acid sequence of SEQ ID NO: GGMWYSDEGSEY (SEQ ID NO: 4572).
6. The ISVD according to any one of item 1 or 2, wherein said ISVD essentially consists of 4 framework regions (FR1 to FR4, respectively) and 3 complementarity determining regions (CDR1 to CDR3 respectively), in which:
   CDR1 (AbM definition) has an amino acid sequence selected from:
      a) the amino acid sequence of GFTLYDHAFG (SEQ ID NO: 4408);
      b) amino acid sequences that have at least 80% amino acid identity with the amino acid sequence of SEQ ID NO: GFTLYDHAFG (SEQ ID NO: 4408); and
      c) amino acid sequences that have 3, 2, or 1 amino acid difference with the amino acid sequences of SEQ ID NO: GFTLYDHAFG (SEQ ID NO: 4408);
and
   CDR2 (AbM definition) has an amino acid sequence selected from:
      a) the amino acid sequence of VISSSGAHEW (SEQ ID NO: 4481);
      b) amino acid sequences that have at least 80% amino acid identity with the amino acid sequence VISSSGAHEW (SEQ ID NO: 4481);
      c) amino acid sequences that have 3, 2, or 1 amino acid difference with the amino acid sequence of VISSSGAHEW (SEQ ID NO: 4481);
and
   CDR3 (AbM definition) has an amino acid sequence selected from:
      a) the amino acid sequence of GGMWYSDEGSEY (SEQ ID NOs: 4572);
      b) amino acid sequences that have at least 80% amino acid identity with the amino acid sequence of SEQ ID NO: GGMWYSDEGSEY (SEQ ID NOs: 4572);
      c) amino acid sequences that have 3, 2, or 1 amino acid difference with the amino acid sequence of SEQ ID NO: GGMWYSDEGSEY (SEQ ID NOs: 4572).
7. The ISVD according to any one of items 1 to 6, in which the amino acid sequences of the CDRs (AbM definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 4056, 4094, 4183, or 4210.
8. The ISVD according to any one of items 1 to 6, in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4524; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4528; or in which
   CDR1 (AbM numbering) consists of the amino acid sequence of SEQ ID NO: 4412, CDR2 (AbM numbering) consists of the amino acid sequence of SEQ ID NO: 4470, CDR3 (AbM numbering) consists of the amino acid sequence of SEQ ID NO: 4528; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4527; or in which
   CDR1 (AbM numbering) consists of the amino acid sequence of SEQ ID NO: 4412, CDR2 (AbM numbering) consists of the amino acid sequence of SEQ ID NO: 4470, CDR3 (AbM numbering) consists of the amino acid sequence of SEQ ID NO: 4527; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4524; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4596; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4528; or in which
   CDR1 (AbM numbering) consists of the amino acid sequence of SEQ ID NO: 4412, CDR2 (AbM numbering) consists of the amino acid sequence of SEQ ID NO: 4470, CDR3 (AbM numbering) consists of the amino acid sequence of SEQ ID NO: 4524; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4524; or in which
   CDR1 (AbM numbering) consists of the amino acid sequence of SEQ ID NO: 4412, CDR2 (AbM numbering) consists of the amino acid sequence of SEQ ID NO: 4470, CDR3 (AbM numbering) consists of the amino acid sequence of SEQ ID NO: 4531; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4531; or in which
   CDR1 (AbM numbering) consists of the amino acid sequence of SEQ ID NO: 4412, CDR2 (AbM numbering) consists of the amino acid sequence of SEQ ID NO: 4470, CDR3 (AbM numbering) consists of the amino acid sequence of SEQ ID NO: 4526; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4526; or in which
   CDR1 (AbM numbering) consists of the amino acid sequence of SEQ ID NO: 4417, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4527; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4531; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4555; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4533; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4532; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4528; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4527; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4605; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4604; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4601; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4603; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4584; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4582; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4555; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4533; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4532; or in which
   CDR1 (AbM numbering) consists of the amino acid sequence of SEQ ID NO: 4411, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4593; or in which
   CDR1 (AbM numbering) consists of the amino acid sequence of SEQ ID NO: 4412, CDR2 (AbM numbering) consists of the amino acid sequence of SEQ ID NO: 4470, CDR3 (AbM numbering) consists of the amino acid sequence of SEQ ID NO: 4593; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4582; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4529; or in which
   CDR1 (AbM numbering) consists of the amino acid sequence of SEQ ID NO: 4429, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4433, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4559; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4547; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4561; or in which
   CDR1 (AbM numbering) consists of the amino acid sequence of SEQ ID NO: 4423, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4432, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4413, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4571; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4562; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4567; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4563; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4564; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4569; or in which
   CDR1 (AbM numbering) consists of the amino acid sequence of SEQ ID NO: 4428, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4426, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4431, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4415, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4607; or in which
   CDR1 (AbM numbering) consists of the amino acid sequence of SEQ ID NO: 4416, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4422, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4410, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4434, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4435, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4436, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4430, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4414, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4420, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4427, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4424, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4425, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4409, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4419, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4418, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4421, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4584; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4597; or in which
   CDR1 (AbM numbering) consists of the amino acid sequence of SEQ ID NO: 4412, CDR2 (AbM numbering) consists of the amino acid sequence of SEQ ID NO: 4490, CDR3 (AbM numbering) consists of the amino acid sequence of SEQ ID NO: 4586; or in which
   CDR1 (AbM numbering) consists of the amino acid sequence of SEQ ID NO: 4412, CDR2 (AbM numbering) consists of the amino acid sequence of SEQ ID NO: 4493, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4492, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4492, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4491, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4491, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4466, CDR3 (AbM numbering) consists of the amino acid sequence of SEQ ID NO: 4586; or in which
   CDR1 (AbM numbering) consists of the amino acid sequence of SEQ ID NO: 4412, CDR2 (AbM numbering) consists of the amino acid sequence of SEQ ID NO: 4467, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4467, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4489, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4466, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4467, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4490, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4587; or in which
   CDR1 (AbM numbering) consists of the amino acid sequence of SEQ ID NO: 4412, CDR2 (AbM numbering) consists of the amino acid sequence of SEQ ID NO: 4489, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4467, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4468, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4490, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4464, CDR3 (AbM numbering) consists of the amino acid sequence of SEQ ID NO: 4586; or in which
   CDR1 (AbM numbering) consists of the amino acid sequence of SEQ ID NO: 4412, CDR2 (AbM numbering) consists of the amino acid sequence of SEQ ID NO: 4463, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4464, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4464, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4463, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4493, CDR3 (AbM numbering) consists of the amino acid sequence of SEQ ID NO: 4586; or in which
   CDR1 (AbM numbering) consists of the amino acid sequence of SEQ ID NO: 4412, CDR2 (AbM numbering) consists of the amino acid sequence of SEQ ID NO: 4493, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4492, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4491, CDR3 (AbM numbering) consists of the amino acid sequence of SEQ ID NO: 4586; or in which
   CDR1 (AbM numbering) consists of the amino acid sequence of SEQ ID NO: 4412, CDR2 (AbM numbering) consists of the amino acid sequence of SEQ ID NO: 4492, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4489, CDR3 (AbM numbering) consists of the amino acid sequence of SEQ ID NO: 4586; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4588; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4589; or in which
   CDR1 (AbM numbering) consists of the amino acid sequence of SEQ ID NO: 4408, CDR2 (AbM numbering) consists of the amino acid sequence of SEQ ID NO: 4466, CDR3 (AbM numbering) consists of the amino acid sequence of SEQ ID NO: 4586; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4590; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4531; or in which
   CDR1 (AbM numbering) consists of the amino acid sequence of SEQ ID NO: 4412, CDR2 (AbM numbering) consists of the amino acid sequence of SEQ ID NO: 4488, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4601; or in which
   CDR1 (AbM numbering) consists of the amino acid sequence of SEQ ID NO: 4412, CDR2 (AbM numbering) consists of the amino acid sequence of SEQ ID NO: 4470, CDR3 (AbM numbering) consists of the amino acid sequence of SEQ ID NO: 4582; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4565; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4596; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4593; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4595; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4584; or in which
   CDR1 (AbM numbering) consists of the amino acid sequence of SEQ ID NO: 4412, CDR2 (AbM numbering) consists of the amino acid sequence of SEQ ID NO: 4470, CDR3 (AbM numbering) consists of the amino acid sequence of SEQ ID NO: 4584; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4582; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4555; or in which
   CDR1 (AbM numbering) consists of the amino acid sequence of SEQ ID NO: 4412, CDR2 (AbM numbering) consists of the amino acid sequence of SEQ ID NO: 4470, CDR3 (AbM numbering) consists of the amino acid sequence of SEQ ID NO: 4555; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4570; or in which
   CDR1 (AbM numbering) consists of the amino acid sequence of SEQ ID NO: 4412, CDR2 (AbM numbering) consists of the amino acid sequence of SEQ ID NO: 4470, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4533; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4532; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4529; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4604; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4605; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4526; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4557; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4526; or in which
   CDR1 (AbM numbering) consists of the amino acid sequence of SEQ ID NO: 4412, CDR2 (AbM numbering) consists of the amino acid sequence of SEQ ID NO: 4485, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4597; or in which
   CDR1 (AbM numbering) consists of the amino acid sequence of SEQ ID NO: 4412, CDR2 (AbM numbering) consists of the amino acid sequence of SEQ ID NO: 4484, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4485, CDR3 (AbM numbering) consists of the amino acid sequence of SEQ ID NO: 4586; or in which
   CDR1 (AbM numbering) consists of the amino acid sequence of SEQ ID NO: 4412, CDR2 (AbM numbering) consists of the amino acid sequence of SEQ ID NO: 4485, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4488, CDR3 (AbM numbering) consists of the amino acid sequence of SEQ ID NO: 4586; or in which
   CDR1 (AbM numbering) consists of the amino acid sequence of SEQ ID NO: 4412, CDR2 (AbM numbering) consists of the amino acid sequence of SEQ ID NO: 4488, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4486, CDR3 (AbM numbering) consists of the amino acid sequence of SEQ ID NO: 4586; or in which
   CDR1 (AbM numbering) consists of the amino acid sequence of SEQ ID NO: 4412, CDR2 (AbM numbering) consists of the amino acid sequence of SEQ ID NO: 4486, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4481, CDR3 (AbM numbering) consists of the amino acid sequence of SEQ ID NO: 4586; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4485, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4486, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4488, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4486, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4487, CDR3 (AbM numbering) consists of the amino acid sequence of SEQ ID NO: 4572; or in which
   CDR1 (AbM numbering) consists of the amino acid sequence of SEQ ID NO: 4408, CDR2 (AbM numbering) consists of the amino acid sequence of SEQ ID NO: 4481, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4603; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4558; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4568; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4566; or in which
   CDR1 (AbM numbering) consists of the amino acid sequence of SEQ ID NO: 4456, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4458, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4451, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4455, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4448, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4392, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4401, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4446, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4450, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4457, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4398, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4388, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4400, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4399, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4397, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4389, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4453, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4393, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4396, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4391, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4407, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4442, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4404, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4405, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4445, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4440, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4441, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4443, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4403, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4438, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4459, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4452, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4447, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4454, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4449, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4460, CDR2 (AbM numbering) consists of the amino acid sequence of SEQ ID NO: 4482, CDR3 (AbM numbering) consists of the amino acid sequence of SEQ ID NO: 4560; or in which
   CDR1 (AbM numbering) consists of the amino acid sequence of SEQ ID NO: 4395, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4394, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4437, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4556; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4554; or in which
   CDR1 (AbM numbering) consists of the amino acid sequence of SEQ ID NO: 4408, CDR2 (AbM numbering) consists of the amino acid sequence of SEQ ID NO: 4463, CDR3 (AbM numbering) consists of the amino acid sequence of SEQ ID NO: 4586; or in which
   CDR1 (AbM numbering) consists of the amino acid sequence of SEQ ID NO: 4408, CDR2 (AbM numbering) consists of the amino acid sequence of SEQ ID NO: 4493, CDR3 (AbM numbering) consists of the amino acid sequence of SEQ ID NO: 4572; or in which
   CDR1 (AbM numbering) consists of the amino acid sequence of SEQ ID NO: 4412, CDR2 (AbM numbering) consists of the amino acid sequence of SEQ ID NO: 4465, CDR3 (AbM numbering) consists of the amino acid sequence of SEQ ID NO: 4586; or in which
   CDR1 (AbM numbering) consists of the amino acid sequence of SEQ ID NO: 4406, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4472, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4552; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4551; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4548; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4541; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4553; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4544; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4550; or in which
   CDR1 (AbM numbering) consists of the amino acid sequence of SEQ ID NO: 4390, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4546; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4549; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4540; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4545; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4542; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4539; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4534; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4522; or in which
   CDR1 (AbM numbering) consists of the amino acid sequence of SEQ ID NO: 4402, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4591; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4602; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4525; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4576; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4575; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4585; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4578; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4574; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4579; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4577; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4580; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4581; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4583; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4573; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4536; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4543; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4538; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4523; or in which
   CDR1 (AbM numbering) consists of the amino acid sequence of SEQ ID NO: 4444, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4470, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4471, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4474, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4483, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4477, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4479, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4476, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4473, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4480, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4469, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4475, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4606; or in which
   CDR1 (AbM numbering) consists of the amino acid sequence of SEQ ID NO: 4439, CDR2 (AbM numbering) consists of the amino acid sequence of SEQ ID NO: 4481, CDR3 (AbM numbering) consists of the amino acid sequence of SEQ ID NO: 4560; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4598; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4537; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4599; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4600; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4530; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4594; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4535; or in which
   CDR1 (AbM numbering) consists of the amino acid sequence of SEQ ID NO: 4412, CDR2 (AbM numbering) consists of the amino acid sequence of SEQ ID NO: 4481, CDR3 (AbM numbering) consists of the amino acid sequence of SEQ ID NO: 4592.
9. The ISVD according to any one of items 1 to 8, which amino acid sequence
   i) has 80% amino acid sequence identity with one of the amino acid sequences of SEQ ID NOs: 4049-4378, in which for the purposes of determining the degree of amino acid identity, the amino acid residues that form the CDR sequences are disregarded;
   and in which:
   ii) preferably one or more of the amino acid residues at positions 11, 37, 44, 45, 47, 83, 84, 103, 104 and 108 according to the Kabat numbering are chosen from the Hallmark residues mentioned in Table 1.
10. The ISVD according to any one of items 1 to 9, which is a humanized ISVD that is chosen from SEQ ID NOs: 4094-4055, or 4057-4378 or from the group consisting of amino acid sequences that have more than 80%, preferably more than 90%, more preferably more than 95%, such as 99% or more amino acid sequence identity with at least one of the amino acid sequences of SEQ ID NOs: 4094-4378.
11. The ISVD according to any one of items 1 to 10, in which the amino acid sequence is chosen from the group consisting of SEQ ID NO: 4056 and SEQ ID NOs: 4094-4055, or 4057-4378.
12. The ISVD according to any one of items 1 to 11, which amino acid sequence
   i) has 80% amino acid sequence identity with one of the amino acid sequences of SEQ ID NOs: 4056, 4094, 4183, or 4210, in which for the purposes of determining the degree of amino acid identity, the amino acid residues that form the CDR sequences are disregarded;
   and in which:
   ii) preferably one or more of the amino acid residues at positions 11, 37, 44, 45, 47, 83, 84, 103, 104 and 108 according to the Kabat numbering are chosen from the Hallmark residues mentioned in Table 1.
13. The ISVD according to any one of items 1 to 12, that essentially consists of a heavy chain variable domain sequence that is derived from a conventional four-chain antibody or that essentially consist of a heavy chain variable domain sequence that is derived from heavy chain antibody.
14. The ISVD according to any one of items 1 to 13, that essentially consists of a VHH, a humanized VHH, a camelized VH, a domain antibody, a single domain antibody, or a dAb.
15. The ISVD according to any one of items 7 to 14, which is a humanized ISVD that is chosen from SEQ ID NO: 4094, 4183, or 4210 or from the group consisting of amino acid sequences that have more than 80%, preferably more than 90%, more preferably more than 95%, such as 99% or more amino acid sequence identity with at least one of the amino acid sequences of SEQ ID NOs: 4056, 4094, 4183, or 4210.
16. The ISVD according to any one of items 7 to 15, in which the amino acid sequence is chosen from the group consisting of SEQ ID NO: 4056 and SEQ ID NOs: 4094, 4183, or 4210.
17. The ISVD according to any one of items 1 to 16, wherein the ISVD specifically binds to human PD-1 with a dissociation constant (K_{D}) of 5×10⁻⁹ to 10⁻¹² moles/litre or less, and preferably 10⁻⁹ to 10⁻¹² moles/litre or less and more preferably 5×10⁻¹⁰ to 1×10⁻¹¹ moles/litre, such as 1.2×10⁻¹⁰ moles/litre, as determined by Surface Plasmon Resonance.
18. The ISVD according to any one of items 1 to 17, wherein the ISVD specifically binds to human PD-1 with a kₒₙ-rate of between 10⁴ M⁻¹ s⁻¹ to about 10⁷ M⁻¹ s⁻¹, preferably between 10⁵ M⁻¹ s⁻¹ and 10⁷ M⁻¹ s⁻¹, more preferably between 10⁵ M⁻¹ s⁻¹ and 10⁶ M⁻¹ s⁻¹, such as between 10⁵ M⁻¹ s⁻¹ and 1×10⁶ M⁻¹ s⁻¹, such as 3.7×10⁵ M⁻¹ s⁻¹ or 3.0×10⁵ M⁻¹ s⁻¹ as determined by Surface Plasmon Resonance.
19. The ISVD according to any one of items 1 to 18, wherein the ISVD specifically binds to human PD-1 with a k_{off} rate between 10³ s⁻¹ (t1/2=0.69 s) and 10⁻⁷ s⁻¹ (providing a near irreversible complex with a t1/2 of multiple days), preferably between 10⁻³ s⁻¹ and 5×10⁻⁶ s⁻¹, more preferably between 5×10⁻⁴ s⁻¹ and 5×10⁻⁶ s⁻¹, such as between 1×10⁻⁴ s⁻¹ and 10⁵ s⁻¹, such as 3.5×10⁻⁵ s⁻¹, as determined by Surface Plasmon Resonance.
20. The ISVD according to any one of items 1 to 19, wherein the ISVD specifically binds to human and cyno PD-1 and does not bind to CD28, CTLA-4, ICOS, and BTLA or other members of the CD28 receptor family.
21. The ISVD according to any one of items 1 to 20, wherein the ISVD antagonizes an activity of PD-L1.
22. The ISVD according to any one of items 1 to 21, wherein the ISVD blocks the interaction of human PD-1 with human PD-L1 with a potency (IC50 value) of 10⁻⁸ M or lower, more preferably of 5×10⁻⁹ M or lower, or even of 10⁹ M or lower, such as between 10⁻¹¹ M and 10⁻⁸ M, between 10⁻¹⁰ M and 10⁻⁹ M, between 10⁻¹⁰ M and 10⁻⁸ M or between 10⁻⁹ M and 10⁻⁸ M, such as 3.08×10⁻⁹ M, for example, as measured in a FACS competition assay on CHO cells expressing human PD-1 with hPD-L1 as a competitor.
23. The ISVD according to any one of items 1 to 22, wherein the ISVD blocks the interaction of cyno PD-1 with cyno PD-L1 with a potency (IC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 10⁻⁹ M, between 10⁻¹⁰ M and 10⁻⁸ M or between 10⁻⁹ M and 10⁻⁸ M, such as 3.57×10⁻⁹ M, for example, as measured in a as measured in a FACS competition assay on CHO cells expressing cyno PD-1 with hPD-L1 as a competitor.
24. The ISVD according to any one of items 1 to 23, wherein the ISVD blocks the binding of hPD-1 to hPD-L1 by at least 50%, such as at least 60%, 70%, 80%, 90%, 95%, 99% or even more, as determined by ligand competition, AlphaScreen, or competitive binding assays (such as competition ELISA or competition FACS).
25. The ISVD according to any one of items 1 to 24, wherein the ISVD induces T-cell activation with a potency (EC50 value) of 10⁻⁷ M or lower, more preferably of 5×10⁻⁸ M or lower, or even of 10⁻⁸ M or lower, such as between 10⁻¹¹ M and 10⁻⁷ M, such as between 10⁻¹⁰ M and 5×10⁻⁸ M, between 10⁻¹⁰ M and 10⁻⁸ M or between 10⁻⁸ M and 10⁻⁷ M, such as 5.68×10⁻⁸ M, 3.60×10⁻⁸ M or 5.8×10⁻⁸ M, as determined in a TT assay.
26. A polypeptide or construct that comprises or essentially consists of one or more ISVDs according to any one of items 1 to 25, and optionally further comprises one or more other groups, residues, moieties or binding units, optionally linked via one or more linkers.
27. The polypeptide or construct according to item 26, in which said one or more other groups, residues, moieties or binding units are amino acid sequences.
28. The polypeptide or construct according to item 26 or 27, in which said one or more linkers are one or more amino acid sequences.
29. The polypeptide or construct according to any one of items 26 to 28, in which said one or more other groups, residues, moieties or binding units are immunoglobulin sequences.
30. The polypeptide or construct according to any one of items 26 to 29, in which said one or more other groups, residues, moieties or binding units are ISVDs.
31. The polypeptide or construct according to any one of items 26 to 30, in which said one or more other groups, residues, moieties or binding units are chosen from the group consisting of VHHs, humanized VHHs, camelized VHs, domain antibodies, single domain antibodies and dAbs.
32. The polypeptide or construct according to any one of items 26 to 31, which is a multivalent construct.
33. The polypeptide or construct according to any one of items 26 to 32, which is a multispecific construct.
34. The polypeptide or construct according to any one of items 26 to 33, in which said one or more other groups, residues, moieties or binding units provide the polypeptide or construct with increased half-life, compared to the ISVD without the one or more other groups, residues, moieties or binding units.
35. The polypeptide or construct according to item 34, in which said one or more other groups, residues, moieties or binding units that provide the polypeptide or construct with increased half-life is chosen from the group consisting of a polyethylene glycol molecule (PEG), serum proteins or fragments thereof, binding units that specifically bind to serum proteins, an Fc portion, and small proteins or peptides that specifically bind to serum proteins.
36. The polypeptide or construct according to item 35, in which said one or more other groups, residues, moieties or binding units that provide the polypeptide or construct with increased half-life is chosen from the group consisting of human serum albumin or fragments thereof.
37. The polypeptide or construct according to item 35, in which said one or more other groups, residues, moieties or binding units that provides the polypeptide or construct with increased half-life are chosen from the group consisting of binding units that specifically bind to serum albumin (such as human serum albumin) or a serum immunoglobulin (such as IgG).
38. The polypeptide or construct according to item 37, in which said one or more other groups, residues, moieties or binding units that provides the polypeptide or construct with increased half-life are chosen from the group consisting of VHHs, humanized VHHs, camelized VHs, domain antibodies, single domain antibodies, or dAbs that specifically bind to serum albumin (such as human serum albumin) or a serum immunoglobulin (such as IgG).
39. The polypeptide or construct according to item 37 or 38, in which said one or more other groups, residues, moieties or binding units that provides the polypeptide or construct with increased half-life is an ISVD that specifically binds human serum albumin. 40. The polypeptide or construct according to item 39, wherein said ISVD that specifically binds human serum albumin essentially consists of 4 framework regions (FR1 to FR4, respectively) and 3 complementarity determining regions (CDR1 to CDR3 respectively), in which:
   CDR1 (AbM numbering) has an amino acid sequence selected from:
      a) the amino acid sequence of SEQ ID NO: 2436;
      b) amino acid sequences that have at least 80% amino acid identity with the amino acid sequence of SEQ ID NO: 2436; and
      c) amino acid sequences that have 3, 2, or 1 amino acid difference with the amino acid sequences of SEQ ID NO: 2436;
and
   CDR2 (AbM numbering) has an amino acid sequence selected from:
      a) the amino acid sequence of SEQ ID NO: 2438;
      b) amino acid sequences that have at least 80% amino acid identity with the amino acid sequence of SEQ ID NO: 2438;
      c) amino acid sequences that have 3, 2, or 1 amino acid difference with the amino acid sequence of SEQ ID NO: 2438;
and
   CDR3 (AbM numbering) has an amino acid sequence selected from:
      a) the amino acid sequence of SEQ ID NOs: 2440;
      b) amino acid sequences that have at least 80% amino acid identity with the amino acid sequence of SEQ ID NO: 2440;
      c) amino acid sequences that have 3, 2, or 1 amino acid difference with the amino acid sequence of SEQ ID NO: 2440.
41. The polypeptide or construct according to item 40, wherein CDR1 consists of the amino acid sequence of SEQ ID NO: 2436, CDR2 consists of the amino acid sequence of SEQ ID NO: 2438, and CDR3 consists of the amino acid sequence of SEQ ID NO: 2440.
42. The polypeptide or construct according to any one of items 39 to 41, wherein said ISVD that specifically binds human serum albumin is selected from the group consisting of ALB8 (SEQ ID NO: 2401), ALB23 (SEQ ID NO: 2402), ALBX00001 (SEQ ID NO: 2415) and ALB23002 (SEQ ID NO: 2416), preferably the ISVD binding to human serum albumin is ALB23002 (SEQ ID NO: 2416).
43. The polypeptide or construct according to any one of items 26 to 42, wherein said linker is chosen from the group consisting of SEQ ID NOs: 2418 to 2434.
44. The polypeptide or construct according to any one of items 26 to 43, further comprising a C-terminal extension.
45. The polypeptide or construct according to item 44, wherein said C-terminal extension is a C-terminal extension (X)ₙ, in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I).
46. A nucleic acid that encodes an ISVD according to any one of items 1 to 25, or a polypeptide according to any one of items 26 to 45.
47. The nucleic acid according to item 45, that is in the form of a genetic construct.
48. A non-human host or host cell that expresses, or that under suitable circumstances is capable of expressing, an ISVD according to any one of items 1 to 25, or a polypeptide according to any one of items 26 to 45; and/or that comprises the nucleic acid according to any one of items 46 or 47.
49. A method for producing an ISVD according to any one of items 1 to 25, or a polypeptide according to any one of items 26 to 45, at least comprising the steps of:
   a) expressing, in a suitable non-human host cell or host organism or in another suitable expression system, a nucleic acid according to any one of items 46 or 47;
   optionally followed by:
   b) isolating and/or purifying the ISVD according to any one of items 1 to 25, or the polypeptide according to any one of items 26 to 45.
50. A method for producing an ISVD according to any one of items 1 to 25, or a polypeptide according to any one of items 26 to 45, said method at least comprising the steps of:
   a) cultivating and/or maintaining a non-human host or host cell according to item 40 under conditions that are such that said non-human host or host cell expresses and/or produces at least one ISVD according to any one of items 1 to 25, or at least one polypeptide according to any one of items 26 to 45;
   optionally followed by:
   b) isolating and/or purifying the ISVD according to any one of items 1 to 25, or polypeptide according to any one of items 26 to 45.
51. A composition comprising at least one ISVD according to any one of items 1 to 25, at least one polypeptide or construct according to any one of items 26 to 45, or at least one nucleic acid according to any one of items 46 or 47.
52. The composition according to item 51, which is a pharmaceutical composition.
53. The composition according to any one of items 51 or 52, which is a pharmaceutical composition, that further comprises at least one pharmaceutically acceptable carrier, diluent or excipient and/or adjuvant, and that optionally comprises one or more further pharmaceutically active polypeptides and/or compounds.
54. The ISVD according to any one of items 1 to 25, the polypeptide or construct according to any one of items 26 to 45, or the composition according to any one of items 51 to 53, for use as a medicament.
55. The ISVD according to any one of items 1 to 25, the polypeptide or construct according to any one of items 26 to 45, or the composition according to any one of items 51 to 53, for use in the diagnosis, prevention and/or treatment of at least one disease and/or disorder.
56. The ISVD according to any one of items 1 to 25, the polypeptide or construct according to any one of items 26 to 45, or the composition according to any one of items 51 to 53, for use in the diagnosis, prevention and/or treatment of at least one disease and/or disorder that is associated with PD-1, with its biological or pharmacological activity, and/or with the biological pathways or signalling in which PD-1 is involved.
57. The ISVD according to any one of items 1 to 25, the polypeptide or construct according to any one of items 26 to 45, or the composition according to any one of items 51 to 53, for use in the diagnosis, prevention and/or treatment of cancer.
58. A method for the diagnosis, prevention and/or treatment of at least one disease and/or disorder, comprising the administration, to a subject in need thereof, of a pharmaceutically active amount of an ISVD according to any one of items 1 to 25, a polypeptide or construct according to any one of items 26 to 45, or a composition according to any one of items 51 to 53.
59. The method according to item 58, for the diagnosis, prevention and/or treatment of at least one disease or disorder that is associated with PD-1, with its biological or pharmacological activity, and/or with the biological pathways or signalling in which PD-1 is involved, said method comprising administering, to a subject in need thereof, a pharmaceutically active amount of at least one ISVD according to any one of items 1 to 25, a polypeptide or construct according to any one of items 26 to 45, or composition according to any one of items 51 to 53.
60. The method according to item 58 or 59, for the diagnosis, prevention and/or treatment of cancer, said method comprising administering, to a subject in need thereof, a pharmaceutically active amount of at least one ISVD according to any one of items 1 to 25, a polypeptide or construct according to any one of items 26 to 45, or a composition according to any one of items 51 to 53.

LENGTHY TABLES
The patent application contains a lengthy table section. A copy of the table is available in electronic form from the USPTO web site (https://seqdata.uspto.gov/docdetail?docId=US20260274953A1). An electronic copy of the table will also be available from the USPTO upon request and payment of the fee set forth in 37 CFR 1.19(b)(3).

1. An immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1 protein, wherein at least one amino acid residue in the ISVD is capable of interacting with at least one amino acid residue in at least one, preferably in each, of the following stretches (of the ectodomain) of human PD-1 protein, when numbered in accordance with SEQ ID NO: 4703: V64-P83, and I126-E136, preferably when the ISVD and human PD-1 protein are mixed in in PBS1× buffer and crystallized by hanging drop vapor diffusion using 30% PEG3350, 50 mM TRIS, pH 8 at 19° C.

2. The ISVD according to claim 1, wherein at least one amino acid residue located in the ISVD is capable of interacting with at least one, at least two, at least three, at least four, at least five, at least six, at least seven, at least ten, at least fifteen, preferably all, of the following amino acid residues of human PD-1 protein, when numbered in accordance with SEQ ID NO: 4703: V64, N66, Y68, S73, N74, Q75, T76, D77, K78, P83, I126, L128, A129, P130, K131, A132, I134, and E136, preferably when the ISVD and human PD-1 protein are mixed in PBS1× buffer and crystallized by hanging drop vapor diffusion using 30% PEG3350, 50 mM TRIS, pH 8 at 19° C.;
wherein the at least one amino acid residue located in the ISVD that is capable of interacting with amino acid residues of human PD-1 protein is located in CDR3;
wherein:
the amino acid residue at position 100e (Kabat numbering) is capable of interacting, preferably by forming a hydrogen bond, with A132 in PD-1;
the amino acid residue at position 100h (Kabat numbering) is capable of interacting, preferably by forming a hydrogen bond, with N66 in PD-1;
the amino acid residue at position 100j (Kabat numbering) is capable of interacting, preferably by forming a hydrogen bond, with N66 in PD-1;
the amino acid residue at position 100j (Kabat numbering) is capable of interacting, preferably by forming a hydrogen bond, with Y68 in PD-1;
the amino acid residue at position 100j (Kabat numbering) is capable of interacting, preferably by forming a hydrogen bond, with A132 in PD-1;
the amino acid residue at position 101 (Kabat numbering) is capable of interacting, preferably by forming a hydrogen bond, with N66 in PD-1; and/or,
the amino acid residue at position 101 (Kabat numbering) is capable of interacting, preferably by forming a hydrogen bond, with K78 in PD-1;
preferably when the ISVD and human PD-1 protein are mixed in PBS1× buffer and crystallized by hanging drop vapor diffusion using 30% PEG3350, 50 mM TRIS, pH 8 at 19° C.;
wherein the interacting amino acid residue(s) in the ISVD and in human PD-1 protein are identified by X-ray crystallography by:
Mixing human PD-1 protein, and the ISVD in PBS1× buffer;
Concentrating the protein complex to 10.6 mg/ml;
Crystallizing by hanging drop vapor diffusion using 30% PEG3350, 50 mM TRIS, pH 8 at 19° C.;
Collecting X-ray diffraction data;
Processing the collected data, or
preferably as described in Example 13; and/or
wherein the (discontinuous) epitope comprises or consists of the following amino acid residues of human PD-1 protein, when numbered in accordance with SEQ ID NO: 4703: V64, N66, Y68, S73, N74, Q75, T76, D77, K78, P83, I126, L128, A129, P130, K131, A132, I134, E136.

3-6. (canceled)

7. The ISVD according to claim 1, that essentially consists of 4 framework regions (FR1 to FR4, respectively) and 3 complementarity determining regions (CDR1 to CDR3, respectively), wherein the CDR3 (AbM definition) consists of the following amino acid sequence X₁X₂STTVRX₃X₄CX₅IMYX₆YDY (SEQ ID NO: 4702), wherein:
X₁ is selected from any amino acid, preferably X₁ is selected from A or G;
X₂ is selected from any amino acid, preferably X₂ is selected T, A, E, G, F, I, D, H, K, S, P, R, Q, Y, V, or L;
X₃ is selected from any amino acid, preferably X₃ is selected from D or G;
X₄ is selected from any amino acid, preferably X₄ is selected from L, M, I, E, A, V, D, H, G, F, K, R, P, S, T, or Y;
X₅ is selected from any amino acid, preferably X₅ is selected from G or F; and
X₆ is selected from any amino acid, preferably X₆is selected from L, V, or F, optionally
wherein the following amino acid residue of the ISVD at the following position: R45 (Kabat numbering) is capable of forming salt bridges with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C.;
wherein at least one, preferably all of the following amino acid residues of the ISVD at the following positions: I100e, Y100h, Y100j, and D101 (Kabat numbering) are capable of forming hydrogen bonds with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C.;
wherein at least one, preferably all, of the following amino acid residues of the ISVD at the following positions: R45, I100e, Y100h, Y100j, and D101, and optionally Q105 (Kabat numbering) is capable of interacting with at least one, preferably all of the following amino acid residues of human PD-1 protein, when numbered in accordance with SEQ ID NO: 4703: N66, Y68, K78, A132, and E136, and optionally N74, preferably wherein:
R45 in the ISVD (Kabat numbering) interacts, preferably by forming a salt bridge, with E136 in human PD-1 protein;
I100e in the ISVD (Kabat numbering) interacts, preferably by forming a hydrogen bond, with A132 in human PD-1 protein;
Y100 h in the ISVD (Kabat numbering) interacts, preferably by forming a hydrogen bond, with N66 in human PD-1 protein;
Y100j in the ISVD (Kabat numbering) interacts, preferably by forming a hydrogen bond, with N66 in human PD-1 protein;
Y100j in the ISVD (Kabat numbering) interacts, preferably by forming a hydrogen bond, with Y68 in human PD-1 protein;
Y100j in the ISVD (Kabat numbering) interacts, preferably by forming a hydrogen bond, with A132 in human PD-1 protein;
D101 in the ISVD (Kabat numbering) interacts, preferably by forming a hydrogen bond, with N66 in human PD-1 protein; and/or
D101 in the ISVD (Kabat numbering) interacts, preferably by forming a hydrogen bond, with K78 in human PD-1 protein;
preferably when the ISVD and human PD-1 protein are mixed in PBS1× buffer and crystallized by hanging drop vapor diffusion using 30% PEG3350, 50 mM TRIS, pH 8 at 19° C.;
wherein said ISVD essentially consists of 4 framework regions (FR1 to FR4, respectively) and 3 complementarity determining regions (CDR1 to CDR3 respectively), in which CDR3 (AbM definition) has an amino acid sequence selected from:
a) the amino acid sequence of SEQ ID NO: 691;
b) amino acid sequences that have at least 80% amino acid identity with the amino acid sequence of SEQ ID NO: 691;
c) amino acid sequences that have 3, 2, or 1 amino acid difference with the amino acid sequence of SEQ ID NO: 691;
wherein the ISVD comprises V2, Q3, L4, R45, W103 and/or Q105 (Kabat numbering);
wherein V2, Q3, L4, R45, I100e, Y100h, Y100j, D101, and Q105 (Kabat numbering) of the ISVD are capable of interacting with the following amino acids of human PD-1 protein, when numbered in accordance with SEQ ID NO: 4703: N66, Y68, S73, N74, Q75, K78, A132, and E136, preferably when the ISVD and human PD-1 protein are mixed in PBS1× buffer and crystallized by hanging drop vapor diffusion using 30% PEG3350, 50 mM TRIS, pH 8 at 19° C.; and/or
wherein the interacting amino acid residue(s) in the ISVD and in human PD-1 protein are identified by X-ray crystallography by:
Mixing human PD-1 protein, and the ISVD in PBS1× buffer;
Concentrating the protein complex to 10.6 mg/ml;
Crystallizing by hanging drop vapor diffusion using 30% PEG3350, 50 mM TRIS, pH 8 at 19° C.;
Collecting X-ray diffraction data;
Processing the collected data, or
preferably as described in Example 13.

8-14. (canceled)

15. The ISVD according to claim 1, that essentially consists of 4 framework regions (FR1 to FR4, respectively) and 3 complementarity determining regions (CDR1 to CDR3, respectively), wherein the ISVD cross-blocks the binding to human PD-1 protein of a polypeptide having an amino acid sequence selected from SEQ ID NOs: 1-488, preferably wherein the ISVD cross-blocks the binding to human PD-1 protein of a polypeptide having an amino acid sequence selected from SEQ ID NO: 402, or wherein the binding of the ISVD to human PD-1 protein is cross-blocked by a polypeptide having an amino acid sequence selected from SEQ ID NOs: 1-488, optionally
wherein the ISVD competes with a polypeptide with an amino acid sequence selected from SEQ ID NO: 1-488 for binding human PD-1 protein, as measured in a competition assay, such as a FACS competition assay; or
an immunoglobulin single variable domain (ISVD) specifically binding human PD-1, that essentially consists of 4 framework regions (FR1 to FR4, respectively) and 3 complementarity determining regions (CDR1 to CDR3, respectively), wherein CDR3 (AbM definition) consists of the amino acid sequence of one of SEQ ID NOs: 644-711, and wherein the amino acid residue of the ISVD at the following position: 45 (Kabat numbering) is capable of forming salt bridges with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C., preferably wherein the ISVD comprises at least the following amino acid residue: R45 (Kabat numbering), optionally
wherein the CDR3 (AbM definition) consists of the amino acid sequence of SEQ ID NO: 691, optionally wherein the CDR1 (AbM definition) consists of the amino acid sequence of SEQ ID NO: 493 and the CDR2 (AbM definition) consists of the amino acid sequence of SEQ ID NO: 526.

16-18. (canceled)

19. The ISVD according to claim 1, that essentially consists of 4 framework regions (FR1 to FR4, respectively) and 3 complementarity determining regions (CDR1 to CDR3, respectively), in which:
CDR1 (AbM definition) has an amino acid sequence selected from:
a) the amino acid sequence of GFTLDX₁YX₂X₃G (SEQ ID NO: 2394), wherein X₁ is selected from A, D, Q, P, S, G, T, V, I, R, K, E, L, H, F, or Y, X₂ is selected from V or F, and X₃ is selected from I, E, D, A, F, G, H, L, K, T, M, S, P, R, Y, V, or Q;
b) amino acid sequences that have at least 80% amino acid identity with the amino acid sequence of GFTLDX₁YX₂X₃G (SEQ ID NO: 2394), wherein X₁ is selected from A, D, Q, P, S, G, T, V, I, R, K, E, L, H, F, or Y, X₂ is selected from V or F, and X₃ is selected from I, E, D, A, F, G, H, L, K, T, M, S, P, R, Y, V, or Q; and
c) amino acid sequences that have 3, 2, or 1 amino acid difference with the amino acid sequences of GFTLDX₁YX₂X₃G (SEQ ID NO: 2394), wherein X₁ is selected from A, D, Q, P, S, G, T, V, I, R, K, E, L, H, F, or Y, X₂ is selected from V or F, and X₃ is selected from I, E, D, A, F, G, H, L, K, T, M, S, P, R, Y, V, or Q;
and:
CDR2 (AbM definition) has an amino acid sequence selected from:
a) the amino acid sequence of CX₁SX₂X₃X₄DRX₅X₆ (SEQ ID NO: 2395), wherein X₁ is selected from L, G, H, K, P, R, S, or T, X₂ is selected from S, E, G, H, P, or Q, and X₃ is selected from N, E, A, D, F, G, H, I, K, L, P, Q, R, S, T, V, or Y, X₄ is selected from G, D, F, H, K, L, P, Q, R, S, T, V, A, or Y, X₅ is selected from I, A, E, G, H, K, P, R, S, or T, and X₆ is selected from N, A, D, E, F, G, H, I, K, L, P, Q, R, S, T, V, or Y;
b) amino acid sequences that have at least 80% amino acid identity with the amino acid sequence of CX₁SX₂X₃X₄DRX₅X₆ (SEQ ID NO: 2395), wherein X₁ is selected from L, G, H, K, P, R, S, or T, X₂ is selected from S, E, G, H, P, or Q, and X₃ is selected from N, E, A, D, F, G, H, I, K, L, P, Q, R, S, T, V, or Y, X₄ is selected from G, D, F, H, K, L, P, Q, R, S, T, V, A, or Y, X₅ is selected from I, A, E, G, H, K, P, R, S, or T, and X₆ is selected from N, A, D, E, F, G, H, I, K, L, P, Q, R, S, T, V, or Y;
c) amino acid sequences that have 3, 2, or 1 amino acid difference with the amino acid sequence of CX₁SX₂X₃X₄DRX₅X₆ (SEQ ID NO: 2395), wherein X₁ is selected from L, G, H, K, P, R, S, or T, X₂ is selected from S, E, G, H, P, or Q, and X₃ is selected from N, E, A, D, F, G, H, I, K, L, P, Q, R, S, T, V, or Y, X₄ is selected from G, D, F, H, K, L, P, Q, R, S, T, V, A, or Y, X₅ is selected from I, A, E, G, H, K, P, R, S, or T, and X₆ is selected from N, A, D, E, F, G, H, I, K, L, P, Q, R, S, T, V, or Y;
and:
CDR3 (AbM definition) has an amino acid sequence selected from:
a) the amino acid sequence of X₁X₂STX₃VRX₄X₅CX₆IX₇YX&YDX9 (SEQ ID NO: 2396), wherein X₁ is selected from A or G, X₂ is selected from T, A, E, G, F, I, D, H, K, S, P, R, Q, Y, V, or L, X₃ is selected from T or I, X₄ is selected from D or G, X₅ is selected from L, M, I, E, A, V, D, H, G, F, L, K, R, P, S, T, or Y, X₆ is selected from G or F, X₇ is selected from I, M, F, Y, L, A, D, E, G, K, H, P, Q, S, T, V, or R, X₈ is selected from L, V, or F, and X₉ is selected from Y, E, I, L, or V;
b) amino acid sequences that have at least 80% amino acid identity with the amino acid sequence of X₁X₂STX₃VRX₄X₅CX₆IX₇YX&YDX9 (SEQ ID NO: 2396), wherein X₁ is selected from A or G, X₂ is selected from T, A, E, G, F, I, D, H, K, S, P, R, Q, Y, V, or L, X₃ is selected from T or I, X₄ is selected from D or G, X₅ is selected from L, M, I, E, A, V, D, H, G, F, L, K, R, P, S, T, or Y, X₆ is selected from G or F, X₇ is selected from I, M, F, Y, L, A, D, E, G, K, H, P, Q, S, T, V, or R, X₈ is selected from L, V, or F, and X₉ is selected from Y, E, I, L, or V;
c) amino acid sequences that have 3, 2, or 1 amino acid difference with the amino acid sequence of X₁X₂STX₃ VRX₄X₅CX₆IX₇YX&YDX9 (SEQ ID NO: 2396), wherein X₁ is selected from A or G, X₂ is selected from T, A, E, G, F, I, D, H, K, S, P, R, Q, Y, V, or L, X₃ is selected from T or I, X₄ is selected from D or G, X₅ is selected from L, M, I, E, A, V, D, H, G, F, L, K, R, P, S, T, or Y, X₆ is selected from G or F, X₇ is selected from I, M, F, Y, L, A, D, E, G, K, H, P, Q, S, T, V, or R, X₈ is selected from L, V, or F, and X₉ is selected from Y, E, I, L, or V; optionally wherein the amino acid sequences of the CDRs (AbM definition) have at least 80% amino acid sequence identity, more preferably at least 90% amino acid sequence identity, such as 95% amino acid sequence identity or 99% amino acid sequence identity or more, or even essentially 100% amino acid sequence identity with the amino acid sequences of the CDRs of the ISVD with the amino acid sequence selected from SEQ ID NOs: 1-488; and/or
wherein said ISVD essentially consists of 4 framework regions (FR1 to FR4, respectively) and 3 complementarity determining regions (CDR1 to CDR3 respectively), in which:
CDR1 (AbM definition) has an amino acid sequence selected from:
a) the amino acid sequence of SEQ ID NO: 493;
b) amino acid sequences that have at least 80% amino acid identity with the amino acid sequence of SEQ ID NO: 493; and
c) amino acid sequences that have 3, 2, or 1 amino acid difference with the amino acid sequences of SEQ ID NO: 493;
and
CDR2 (AbM definition) has an amino acid sequence selected from:
a) the amino acid sequence of SEQ ID NO: 526;
b) amino acid sequences that have at least 80% amino acid identity with the amino acid sequence of SEQ ID NO: 526;
c) amino acid sequences that have 3, 2, or 1 amino acid difference with the amino acid sequence of SEQ ID NO: 526;
and
CDR3 (AbM definition) has an amino acid sequence selected from:
a) the amino acid sequence of SEQ ID NO: 691;
b) amino acid sequences that have at least 80% amino acid identity with the amino acid sequence of SEQ ID NO: 691;
amino acid sequences that have 3, 2, or 1 amino acid difference with the amino acid sequence of SEQ ID NO: 691; or
an ISVD that essentially consists of 4 framework regions (FR1 to FR4, respectively) and 3 complementarity determining regions (CDR1 to CDR3, respectively), wherein
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 546, CDR3 (AbM definition) consists of the amino acid sequence of SEQ ID NO: 680; or in which
CDR1 (AbM definition) consists of the amino acid sequence of SEQ ID NO: 494, CDR2 (AbM definition) consists of the amino acid sequence of SEQ ID NO: 547, CDR3 (AbM definition) consists of the amino acid sequence of SEQ ID NO: 674; or in which
CDR1 (AbM definition) consists of the amino acid sequence of SEQ ID NO: 495, CDR2 (AbM definition) consists of the amino acid sequence of SEQ ID NO: 547, CDR3 (AbM definition) consists of the amino acid sequence of SEQ ID NO: 674; or in which
CDR1 (AbM definition) consists of the amino acid sequence of SEQ ID NO: 496, CDR2 (AbM definition) consists of the amino acid sequence of SEQ ID NO: 547, CDR3 (AbM definition) consists of the amino acid sequence of SEQ ID NO: 674; or in which
CDR1 (AbM definition) consists of the amino acid sequence of SEQ ID NO: 493, CDR2 (AbM definition) consists of the amino acid sequence of SEQ ID NO: 547, CDR3 (AbM definition) consists of the amino acid sequence of SEQ ID NO: 674; or in which
CDR1 (AbM definition) consists of the amino acid sequence of SEQ ID NO: 494, CDR2 (AbM definition) consists of the amino acid sequence of SEQ ID NO: 541, CDR3 (AbM definition) consists of the amino acid sequence of SEQ ID NO: 674; or in which
CDR1 (AbM definition) consists of the amino acid sequence of SEQ ID NO: 495, CDR2 (AbM definition) consists of the amino acid sequence of SEQ ID NO: 541, CDR3 (AbM definition) consists of the amino acid sequence of SEQ ID NO: 674; or in which
CDR1 (AbM definition) consists of the amino acid sequence of SEQ ID NO: 496, CDR2 (AbM definition) consists of the amino acid sequence of SEQ ID NO: 541, CDR3 (AbM definition) consists of the amino acid sequence of SEQ ID NO: 674; or in which
CDR1 (AbM definition) consists of the amino acid sequence of SEQ ID NO: 493, CDR2 (AbM definition) consists of the amino acid sequence of SEQ ID NO: 541, CDR3 (AbM definition) consists of the amino acid sequence of SEQ ID NO: 674; or in which
CDR1 (AbM definition) consists of the amino acid sequence of SEQ ID NO: 494, CDR2 (AbM definition) consists of the amino acid sequence of SEQ ID NO: 549, CDR3 (AbM definition) consists of the amino acid sequence of SEQ ID NO: 674; or in which
CDR1 (AbM definition) consists of the amino acid sequence of SEQ ID NO: 495, CDR2 (AbM definition) consists of the amino acid sequence of SEQ ID NO: 549, CDR3 (AbM definition) consists of the amino acid sequence of SEQ ID NO: 674; or in which
CDR1 (AbM definition) consists of the amino acid sequence of SEQ ID NO: 496, CDR2 (AbM definition) consists of the amino acid sequence of SEQ ID NO: 549, CDR3 (AbM definition) consists of the amino acid sequence of SEQ ID NO: 674; or in which
CDR1 (AbM definition) consists of the amino acid sequence of SEQ ID NO: 493, CDR2 (AbM definition) consists of the amino acid sequence of SEQ ID NO: 548, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 549, CDR3 (AbM definition) consists of the amino acid sequence of SEQ ID NO: 674; or in which
CDR1 (AbM definition) consists of the amino acid sequence of SEQ ID NO: 494, CDR2 (AbM definition) consists of the amino acid sequence of SEQ ID NO: 547, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 540, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 542, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 544, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 530, CDR3 (AbM definition) consists of the amino acid sequence of SEQ ID NO: 680; or in which
CDR1 (AbM definition) consists of the amino acid sequence of SEQ ID NO: 495, CDR2 (AbM definition) consists of the amino acid sequence of SEQ ID NO: 604, CDR3 (AbM definition) consists of the amino acid sequence of SEQ ID NO: 680; or in which
CDR1 (AbM definition) consists of the amino acid sequence of SEQ ID NO: 496, CDR2 (AbM definition) consists of the amino acid sequence of SEQ ID NO: 604, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 604, CDR3 (AbM definition) consists of the amino acid sequence of SEQ ID NO: 680; or in which
CDR1 (AbM definition) consists of the amino acid sequence of SEQ ID NO: 494, CDR2 (AbM definition) consists of the amino acid sequence of SEQ ID NO: 603, CDR3 (AbM definition) consists of the amino acid sequence of SEQ ID NO: 680; or in which
CDR1 (AbM definition) consists of the amino acid sequence of SEQ ID NO: 495, CDR2 (AbM definition) consists of the amino acid sequence of SEQ ID NO: 603, CDR3 (AbM definition) consists of the amino acid sequence of SEQ ID NO: 680; or in which
CDR1 (AbM definition) consists of the amino acid sequence of SEQ ID NO: 496, CDR2 (AbM definition) consists of the amino acid sequence of SEQ ID NO: 603, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 603, CDR3 (AbM definition) consists of the amino acid sequence of SEQ ID NO: 680; or in which
CDR1 (AbM definition) consists of the amino acid sequence of SEQ ID NO: 494, CDR2 (AbM definition) consists of the amino acid sequence of SEQ ID NO: 543, CDR3 (AbM definition) consists of the amino acid sequence of SEQ ID NO: 680; or in which
CDR1 (AbM definition) consists of the amino acid sequence of SEQ ID NO: 496, CDR2 (AbM definition) consists of the amino acid sequence of SEQ ID NO: 543, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 680; or in which
CDR1 (AbM definition) consists of the amino acid sequence of SEQ ID NO: 494, CDR2 (AbM definition) consists of the amino acid sequence of SEQ ID NO: 604, CDR3 (AbM definition) consists of the amino acid sequence of SEQ ID NO: 680; or in which
CDR1 (AbM definition) consists of the amino acid sequence of SEQ ID NO: 495, CDR2 (AbM definition) consists of the amino acid sequence of SEQ ID NO: 543, CDR3 (AbM definition) consists of the amino acid sequence of SEQ ID NO: 680; or in which
CDR1 (AbM definition) consists of the amino acid sequence of SEQ ID NO: 495, CDR2 (AbM definition) consists of the amino acid sequence of SEQ ID NO: 547, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 535, CDR3 (AbM definition) consists of the amino acid sequence of SEQ ID NO: 680; or in which
CDR1 (AbM definition) consists of the amino acid sequence of SEQ ID NO: 496, CDR2 (AbM definition) consists of the amino acid sequence of SEQ ID NO: 547, CDR3 (AbM definition) consists of the amino acid sequence of SEQ ID NO: 680; or in which
CDR1 (AbM definition) consists of the amino acid sequence of SEQ ID NO: 494, CDR2 (AbM definition) consists of the amino acid sequence of SEQ ID NO: 541, CDR3 (AbM definition) consists of the amino acid sequence of SEQ ID NO: 680; or in which
CDR1 (AbM definition) consists of the amino acid sequence of SEQ ID NO: 496, CDR2 (AbM definition) consists of the amino acid sequence of SEQ ID NO: 531, CDR3 (AbM definition) consists of the amino acid sequence of SEQ ID NO: 674; or in which
CDR1 (AbM definition) consists of the amino acid sequence of SEQ ID NO: 493, CDR2 (AbM definition) consists of the amino acid sequence of SEQ ID NO: 531, CDR3 (AbM definition) consists of the amino acid sequence of SEQ ID NO: 674; or in which
CDR1 (AbM definition) consists of the amino acid sequence of SEQ ID NO: 494, CDR2 (AbM definition) consists of the amino acid sequence of SEQ ID NO: 534, CDR3 (AbM definition) consists of the amino acid sequence of SEQ ID NO: 680; or in which
CDR1 (AbM definition) consists of the amino acid sequence of SEQ ID NO: 495, CDR2 (AbM definition) consists of the amino acid sequence of SEQ ID NO: 534, CDR3 (AbM definition) consists of the amino acid sequence of SEQ ID NO: 680; or in which
CDR1 (AbM definition) consists of the amino acid sequence of SEQ ID NO: 496, CDR2 (AbM definition) consists of the amino acid sequence of SEQ ID NO: 534, CDR3 (AbM definition) consists of the amino acid sequence of SEQ ID NO: 680; or in which
CDR1 (AbM definition) consists of the amino acid sequence of SEQ ID NO: 494, CDR2 (AbM definition) consists of the amino acid sequence of SEQ ID NO: 531, CDR3 (AbM definition) consists of the amino acid sequence of SEQ ID NO: 680; or in which
CDR1 (AbM definition) consists of the amino acid sequence of SEQ ID NO: 495, CDR2 (AbM definition) consists of the amino acid sequence of SEQ ID NO: 531, CDR3 (AbM definition) consists of the amino acid sequence of SEQ ID NO: 680; or in which
CDR1 (AbM definition) consists of the amino acid sequence of SEQ ID NO: 496, CDR2 (AbM definition) consists of the amino acid sequence of SEQ ID NO: 531, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 531, CDR3 (AbM definition) consists of the amino acid sequence of SEQ ID NO: 680; or in which
CDR1 (AbM definition) consists of the amino acid sequence of SEQ ID NO: 495, CDR2 (AbM definition) consists of the amino acid sequence of SEQ ID NO: 531, CDR3 (AbM definition) consists of the amino acid sequence of SEQ ID NO: 674; or in which
CDR1 (AbM definition) consists of the amino acid sequence of SEQ ID NO: 493, CDR2 (AbM definition) consists of the amino acid sequence of SEQ ID NO: 529, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 602, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 527, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 528, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 601, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 532, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 534, CDR3 (AbM definition) consists of the amino acid sequence of SEQ ID NO: 680; or in which
CDR1 (AbM definition) consists of the amino acid sequence of SEQ ID NO: 494, CDR2 (AbM definition) consists of the amino acid sequence of SEQ ID NO: 531, CDR3 (AbM definition) consists of the amino acid sequence of SEQ ID NO: 674; or in which
CDR1 (AbM definition) consists of the amino acid sequence of SEQ ID NO: 493, CDR2 (AbM definition) consists of the amino acid sequence of SEQ ID NO: 534, CDR3 (AbM definition) consists of the amino acid sequence of SEQ ID NO: 674; or in which
CDR1 (AbM definition) consists of the amino acid sequence of SEQ ID NO: 496, CDR2 (AbM definition) consists of the amino acid sequence of SEQ ID NO: 534, CDR3 (AbM definition) consists of the amino acid sequence of SEQ ID NO: 674; or in which
CDR1 (AbM definition) consists of the amino acid sequence of SEQ ID NO: 495, CDR2 (AbM definition) consists of the amino acid sequence of SEQ ID NO: 541, CDR3 (AbM definition) consists of the amino acid sequence of SEQ ID NO: 680; or in which
CDR1 (AbM definition) consists of the amino acid sequence of SEQ ID NO: 496, CDR2 (AbM definition) consists of the amino acid sequence of SEQ ID NO: 541, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 541, CDR3 (AbM definition) consists of the amino acid sequence of SEQ ID NO: 680; or in which
CDR1 (AbM definition) consists of the amino acid sequence of SEQ ID NO: 494, CDR2 (AbM definition) consists of the amino acid sequence of SEQ ID NO: 549, CDR3 (AbM definition) consists of the amino acid sequence of SEQ ID NO: 680; or in which
CDR1 (AbM definition) consists of the amino acid sequence of SEQ ID NO: 496, CDR2 (AbM definition) consists of the amino acid sequence of SEQ ID NO: 549, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 549, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 547, CDR3 (AbM definition) consists of the amino acid sequence of SEQ ID NO: 680; or in which
CDR1 (AbM definition) consists of the amino acid sequence of SEQ ID NO: 495, CDR2 (AbM definition) consists of the amino acid sequence of SEQ ID NO: 549, CDR3 (AbM definition) consists of the amino acid sequence of SEQ ID NO: 680; or in which
CDR1 (AbM definition) consists of the amino acid sequence of SEQ ID NO: 494, CDR2 (AbM definition) consists of the amino acid sequence of SEQ ID NO: 534, CDR3 (AbM definition) consists of the amino acid sequence of SEQ ID NO: 674; or in which
CDR1 (AbM definition) consists of the amino acid sequence of SEQ ID NO: 495, CDR2 (AbM definition) consists of the amino acid sequence of SEQ ID NO: 534, CDR3 (AbM definition) consists of the amino acid sequence of SEQ ID NO: 674; or in which
CDR1 (AbM definition) consists of the amino acid sequence of SEQ ID NO: 494, CDR2 (AbM definition) consists of the amino acid sequence of SEQ ID NO: 543, CDR3 (AbM definition) consists of the amino acid sequence of SEQ ID NO: 674; or in which
CDR1 (AbM definition) consists of the amino acid sequence of SEQ ID NO: 493, CDR2 (AbM definition) consists of the amino acid sequence of SEQ ID NO: 578, CDR3 (AbM definition) consists of the amino acid sequence of SEQ ID NO: 707; or in which
CDR1 (AbM definition) consists of the amino acid sequence of SEQ ID NO: 493, CDR2 (AbM definition) consists of the amino acid sequence of SEQ ID NO: 578, CDR3 (AbM definition) consists of the amino acid sequence of SEQ ID NO: 703; or in which
CDR1 (AbM definition) consists of the amino acid sequence of SEQ ID NO: 493, CDR2 (AbM definition) consists of the amino acid sequence of SEQ ID NO: 578, CDR3 (AbM definition) consists of the amino acid sequence of SEQ ID NO: 672; or in which
CDR1 (AbM definition) consists of the amino acid sequence of SEQ ID NO: 493, CDR2 (AbM definition) consists of the amino acid sequence of SEQ ID NO: 578, CDR3 (AbM definition) consists of the amino acid sequence of SEQ ID NO: 665; or in which
CDR1 (AbM definition) consists of the amino acid sequence of SEQ ID NO: 493, CDR2 (AbM definition) consists of the amino acid sequence of SEQ ID NO: 592, CDR3 (AbM definition) consists of the amino acid sequence of SEQ ID NO: 701; or in which
CDR1 (AbM definition) consists of the amino acid sequence of SEQ ID NO: 493, CDR2 (AbM definition) consists of the amino acid sequence of SEQ ID NO: 592, CDR3 (AbM definition) consists of the amino acid sequence of SEQ ID NO: 703; or in which
CDR1 (AbM definition) consists of the amino acid sequence of SEQ ID NO: 493, CDR2 (AbM definition) consists of the amino acid sequence of SEQ ID NO: 592, CDR3 (AbM definition) consists of the amino acid sequence of SEQ ID NO: 672; or in which
CDR1 (AbM definition) consists of the amino acid sequence of SEQ ID NO: 493, CDR2 (AbM definition) consists of the amino acid sequence of SEQ ID NO: 592, CDR3 (AbM definition) consists of the amino acid sequence of SEQ ID NO: 665; or in which
CDR1 (AbM definition) consists of the amino acid sequence of SEQ ID NO: 493, CDR2 (AbM definition) consists of the amino acid sequence of SEQ ID NO: 592, CDR3 (AbM definition) consists of the amino acid sequence of SEQ ID NO: 707; or in which
CDR1 (AbM definition) consists of the amino acid sequence of SEQ ID NO: 493, CDR2 (AbM definition) consists of the amino acid sequence of SEQ ID NO: 593, CDR3 (AbM definition) consists of the amino acid sequence of SEQ ID NO: 701; or in which
CDR1 (AbM definition) consists of the amino acid sequence of SEQ ID NO: 493, CDR2 (AbM definition) consists of the amino acid sequence of SEQ ID NO: 593, CDR3 (AbM definition) consists of the amino acid sequence of SEQ ID NO: 703; or in which
CDR1 (AbM definition) consists of the amino acid sequence of SEQ ID NO: 493, CDR2 (AbM definition) consists of the amino acid sequence of SEQ ID NO: 593, CDR3 (AbM definition) consists of the amino acid sequence of SEQ ID NO: 672; or in which
CDR1 (AbM definition) consists of the amino acid sequence of SEQ ID NO: 493, CDR2 (AbM definition) consists of the amino acid sequence of SEQ ID NO: 593, CDR3 (AbM definition) consists of the amino acid sequence of SEQ ID NO: 665; or in which
CDR1 (AbM definition) consists of the amino acid sequence of SEQ ID NO: 493, CDR2 (AbM definition) consists of the amino acid sequence of SEQ ID NO: 593, CDR3 (AbM definition) consists of the amino acid sequence of SEQ ID NO: 707; or in which
CDR1 (AbM definition) consists of the amino acid sequence of SEQ ID NO: 493, CDR2 (AbM definition) consists of the amino acid sequence of SEQ ID NO: 594, CDR3 (AbM definition) consists of the amino acid sequence of SEQ ID NO: 701; or in which
CDR1 (AbM definition) consists of the amino acid sequence of SEQ ID NO: 493, CDR2 (AbM definition) consists of the amino acid sequence of SEQ ID NO: 594, CDR3 (AbM definition) consists of the amino acid sequence of SEQ ID NO: 703; or in which
CDR1 (AbM definition) consists of the amino acid sequence of SEQ ID NO: 494, CDR2 (AbM definition) consists of the amino acid sequence of SEQ ID NO: 537, CDR3 (AbM definition) consists of the amino acid sequence of SEQ ID NO: 670; or in which
CDR1 (AbM definition) consists of the amino acid sequence of SEQ ID NO: 494, CDR2 (AbM definition) consists of the amino acid sequence of SEQ ID NO: 593, CDR3 (AbM definition) consists of the amino acid sequence of SEQ ID NO: 670; or in which
CDR1 (AbM definition) consists of the amino acid sequence of SEQ ID NO: 493, CDR2 (AbM definition) consists of the amino acid sequence of SEQ ID NO: 559, CDR3 (AbM definition) consists of the amino acid sequence of SEQ ID NO: 645; or in which
CDR1 (AbM definition) consists of the amino acid sequence of SEQ ID NO: 494, CDR2 (AbM definition) consists of the amino acid sequence of SEQ ID NO: 553, CDR3 (AbM definition) consists of the amino acid sequence of SEQ ID NO: 670; or in which
CDR1 (AbM definition) consists of the amino acid sequence of SEQ ID NO: 493, CDR2 (AbM definition) consists of the amino acid sequence of SEQ ID NO: 598, CDR3 (AbM definition) consists of the amino acid sequence of SEQ ID NO: 645; or in which
CDR1 (AbM definition) consists of the amino acid sequence of SEQ ID NO: 493, CDR2 (AbM definition) consists of the amino acid sequence of SEQ ID NO: 593, CDR3 (AbM definition) consists of the amino acid sequence of SEQ ID NO: 644; or in which
CDR1 (AbM definition) consists of the amino acid sequence of SEQ ID NO: 493, CDR2 (AbM definition) consists of the amino acid sequence of SEQ ID NO: 557, CDR3 (AbM definition) consists of the amino acid sequence of SEQ ID NO: 645; or in which
CDR1 (AbM definition) consists of the amino acid sequence of SEQ ID NO: 494, CDR2 (AbM definition) consists of the amino acid sequence of SEQ ID NO: 550, CDR3 (AbM definition) consists of the amino acid sequence of SEQ ID NO: 670; or in which
CDR1 (AbM definition) consists of the amino acid sequence of SEQ ID NO: 494, CDR2 (AbM definition) consists of the amino acid sequence of SEQ ID NO: 595, CDR3 (AbM definition) consists of the amino acid sequence of SEQ ID NO: 670; or in which
CDR1 (AbM definition) consists of the amino acid sequence of SEQ ID NO: 494, CDR2 (AbM definition) consists of the amino acid sequence of SEQ ID NO: 555, CDR3 (AbM definition) consists of the amino acid sequence of SEQ ID NO: 670; or in which
CDR1 (AbM definition) consists of the amino acid sequence of SEQ ID NO: 494, CDR2 (AbM definition) consists of the amino acid sequence of SEQ ID NO: 559, CDR3 (AbM definition) consists of the amino acid sequence of SEQ ID NO: 670; or in which
CDR1 (AbM definition) consists of the amino acid sequence of SEQ ID NO: 493, CDR2 (AbM definition) consists of the amino acid sequence of SEQ ID NO: 550, CDR3 (AbM definition) consists of the amino acid sequence of SEQ ID NO: 645; or in which
CDR1 (AbM definition) consists of the amino acid sequence of SEQ ID NO: 493, CDR2 (AbM definition) consists of the amino acid sequence of SEQ ID NO: 553, CDR3 (AbM definition) consists of the amino acid sequence of SEQ ID NO: 645; or in which
CDR1 (AbM definition) consists of the amino acid sequence of SEQ ID NO: 494, CDR2 (AbM definition) consists of the amino acid sequence of SEQ ID NO: 591, CDR3 (AbM definition) consists of the amino acid sequence of SEQ ID NO: 670; or in which
CDR1 (AbM definition) consists of the amino acid sequence of SEQ ID NO: 493, CDR2 (AbM definition) consists of the amino acid sequence of SEQ ID NO: 578, CDR3 (AbM definition) consists of the amino acid sequence of SEQ ID NO: 701; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 701; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 703; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 672; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 665; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 707; or in which
CDR1 (AbM definition) consists of the amino acid sequence of SEQ ID NO: 493, CDR2 (AbM definition) consists of the amino acid sequence of SEQ ID NO: 594, CDR3 (AbM definition) consists of the amino acid sequence of SEQ ID NO: 672; or in which
CDR1 (AbM definition) consists of the amino acid sequence of SEQ ID NO: 493, CDR2 (AbM definition) consists of the amino acid sequence of SEQ ID NO: 603, CDR3 (AbM definition) consists of the amino acid sequence of SEQ ID NO: 674; or in which
CDR1 (AbM definition) consists of the amino acid sequence of SEQ ID NO: 496, CDR2 (AbM definition) consists of the amino acid sequence of SEQ ID NO: 543, CDR3 (AbM definition) consists of the amino acid sequence of SEQ ID NO: 674; or in which
CDR1 (AbM definition) consists of the amino acid sequence of SEQ ID NO: 493, CDR2 (AbM definition) consists of the amino acid sequence of SEQ ID NO: 539, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 678; or in which
CDR1 (AbM definition) consists of the amino acid sequence of SEQ ID NO: 494, CDR2 (AbM definition) consists of the amino acid sequence of SEQ ID NO: 604, CDR3 (AbM definition) consists of the amino acid sequence of SEQ ID NO: 674; or in which
CDR1 (AbM definition) consists of the amino acid sequence of SEQ ID NO: 495, CDR2 (AbM definition) consists of the amino acid sequence of SEQ ID NO: 604, CDR3 (AbM definition) consists of the amino acid sequence of SEQ ID NO: 674; or in which
CDR1 (AbM definition) consists of the amino acid sequence of SEQ ID NO: 496, CDR2 (AbM definition) consists of the amino acid sequence of SEQ ID NO: 604, CDR3 (AbM definition) consists of the amino acid sequence of SEQ ID NO: 674; or in which
CDR1 (AbM definition) consists of the amino acid sequence of SEQ ID NO: 493, CDR2 (AbM definition) consists of the amino acid sequence of SEQ ID NO: 604, CDR3 (AbM definition) consists of the amino acid sequence of SEQ ID NO: 674; or in which
CDR1 (AbM definition) consists of the amino acid sequence of SEQ ID NO: 494, CDR2 (AbM definition) consists of the amino acid sequence of SEQ ID NO: 603, CDR3 (AbM definition) consists of the amino acid sequence of SEQ ID NO: 674; or in which
CDR1 (AbM definition) consists of the amino acid sequence of SEQ ID NO: 495, CDR2 (AbM definition) consists of the amino acid sequence of SEQ ID NO: 603, CDR3 (AbM definition) consists of the amino acid sequence of SEQ ID NO: 674; or in which
CDR1 (AbM definition) consists of the amino acid sequence of SEQ ID NO: 495, CDR2 (AbM definition) consists of the amino acid sequence of SEQ ID NO: 543, CDR3 (AbM definition) consists of the amino acid sequence of SEQ ID NO: 674; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 674; or in which
CDR1 (AbM definition) consists of the amino acid sequence of SEQ ID NO: 496, CDR2 (AbM definition) consists of the amino acid sequence of SEQ ID NO: 603, CDR3 (AbM definition) consists of the amino acid sequence of SEQ ID NO: 674; or in which
CDR1 (AbM definition) consists of the amino acid sequence of SEQ ID NO: 493, CDR2 (AbM definition) consists of the amino acid sequence of SEQ ID NO: 594, CDR3 (AbM definition) consists of the amino acid sequence of SEQ ID NO: 707; or in which
CDR1 (AbM definition) consists of the amino acid sequence of SEQ ID NO: 493, CDR2 (AbM definition) consists of the amino acid sequence of SEQ ID NO: 594, CDR3 (AbM definition) consists of the amino acid sequence of SEQ ID NO: 665; or in which
CDR1 (AbM definition) consists of the amino acid sequence of SEQ ID NO: 493, CDR2 (AbM definition) consists of the amino acid sequence of SEQ ID NO: 595, CDR3 (AbM definition) consists of the amino acid sequence of SEQ ID NO: 701; or in which
CDR1 (AbM definition) consists of the amino acid sequence of SEQ ID NO: 493, CDR2 (AbM definition) consists of the amino acid sequence of SEQ ID NO: 595, CDR3 (AbM definition) consists of the amino acid sequence of SEQ ID NO: 703; or in which
CDR1 (AbM definition) consists of the amino acid sequence of SEQ ID NO: 493, CDR2 (AbM definition) consists of the amino acid sequence of SEQ ID NO: 595, CDR3 (AbM definition) consists of the amino acid sequence of SEQ ID NO: 672; or in which
CDR1 (AbM definition) consists of the amino acid sequence of SEQ ID NO: 493, CDR2 (AbM definition) consists of the amino acid sequence of SEQ ID NO: 595, CDR3 (AbM definition) consists of the amino acid sequence of SEQ ID NO: 665; or in which
CDR1 (AbM definition) consists of the amino acid sequence of SEQ ID NO: 493, CDR2 (AbM definition) consists of the amino acid sequence of SEQ ID NO: 595, CDR3 (AbM definition) consists of the amino acid sequence of SEQ ID NO: 707; or in which
CDR1 (AbM definition) consists of the amino acid sequence of SEQ ID NO: 494, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 494, CDR2 (AbM definition) consists of the amino acid sequence of SEQ ID NO: 542, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 676; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 677; or in which
CDR1 (AbM definition) consists of the amino acid sequence of SEQ ID NO: 493, CDR2 (AbM definition) consists of the amino acid sequence of SEQ ID NO: 575, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 570, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 566, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 564, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 567, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 572, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 580, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 587, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 588, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 583, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 568, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 584, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 560, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 586, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 2393, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 585, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 561, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 582, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 581, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 592, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 552, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 558, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 590, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 597, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 579, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 577, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 571, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 567, CDR3 (AbM definition) consists of the amino acid sequence of SEQ ID NO: 690; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 702; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 667; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 673; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 663; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 666; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 661; or in which
CDR1 (AbM definition) consists of the amino acid sequence of SEQ ID NO: 493, CDR2 (AbM definition) consists of the amino acid sequence of SEQ ID NO: 576, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 573, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 562, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 574, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 563, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 556, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 589, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 599, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 681; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 662; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 538, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 594, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 554, CDR3 (AbM definition) consists of the amino acid sequence of SEQ ID NO: 691; or in which
CDR1 (AbM definition) consists of the amino acid sequence of SEQ ID NO: 497, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 498, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 499, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 500, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 501, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 502, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 503, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 504, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 505, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 506, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 507, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 508, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 509, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 510, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 511, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 536, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 700; or in which
CDR1 (AbM definition) consists of the amino acid sequence of SEQ ID NO: 493, CDR2 (AbM definition) consists of the amino acid sequence of SEQ ID NO: 569, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 565, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 675; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 687; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 706; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 689; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 669; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 664; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 704; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 684; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 665; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 707; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 704; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 670; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 664; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 675; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 649; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 650; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 660; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 648; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 710; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 711; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 657; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 705; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 653; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 647; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 651; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 679; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 705; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 706; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 680; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 665; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 707; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 689; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 687; or in which
CDR1 (AbM definition) consists of the amino acid sequence of SEQ ID NO: 493, CDR2 (AbM definition) consists of the amino acid sequence of SEQ ID NO: 551, CDR3 (AbM definition) consists of the amino acid sequence of SEQ ID NO: 684; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 655; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 699; or in which
CDR1 (AbM definition) consists of the amino acid sequence of SEQ ID NO: 493, CDR2 (AbM definition) consists of the amino acid sequence of SEQ ID NO: 543, CDR3 (AbM definition) consists of the amino acid sequence of SEQ ID NO: 671; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 683; or in which
CDR1 (AbM definition) consists of the amino acid sequence of SEQ ID NO: 2392, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 512, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 513, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 692; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 694; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 689; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 688; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 695; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 682; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 687; or in which
CDR1 (AbM definition) consists of the amino acid sequence of SEQ ID NO: 494, CDR2 (AbM definition) consists of the amino acid sequence of SEQ ID NO: 600, CDR3 (AbM definition) consists of the amino acid sequence of SEQ ID NO: 670; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 696; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 685; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 686; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 697; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 698; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 684; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 708; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 709; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 668; or in which
CDR1 (AbM definition) consists of the amino acid sequence of SEQ ID NO: 494, CDR2 (AbM definition) consists of the amino acid sequence of SEQ ID NO: 557, CDR3 (AbM definition) consists of the amino acid sequence of SEQ ID NO: 670; or in which
CDR1 (AbM definition) consists of the amino acid sequence of SEQ ID NO: 514, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 693; or in which
CDR1 (AbM definition) consists of the amino acid sequence of SEQ ID NO: 496, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 646; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 652; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 659; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 658; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 656; or in which
CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 654; or in which
CDR1 (AbM definition) consists of the amino acid sequence of SEQ ID NO: 515, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 516, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 517, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 518, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 519, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 520, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 495, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 521, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 522, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 523, CDR2 (AbM definition) consists of the amino acid sequence of SEQ ID NO: 526, CDR3 (AbM definition) consists of the amino acid sequence of SEQ ID NO: 691; or in which
CDR1 (AbM definition) consists of the amino acid sequence of SEQ ID NO: 494, CDR2 (AbM definition) consists of the amino acid sequence of SEQ ID NO: 596, CDR3 (AbM definition) consists of the amino acid sequence of SEQ ID NO: 670;
wherein the CDR1 (AbM definition) consists of the amino acid sequence of SEQ ID NO: 493, CDR2 (AbM definition) consists of the amino acid sequence of SEQ ID NO: 526, CDR3 (AbM definition) consists of the amino acid sequence of SEQ ID NO: 691; or CDR1 (AbM definition) consists of the amino acid sequence of SEQ ID NO: 493, CDR2 (AbM definition) consists of the amino acid sequence of SEQ ID NO: 549, CDR3 (AbM definition) consists of the amino acid sequence of SEQ ID NO: 680;
wherein the ISVD comprises V2, Q3, L4, R45, W103 and/or Q105 (Kabat numbering);
wherein the amino acid residue of the ISVD at the following position: 45 (Kabat numbering) is capable of forming salt bridges with at least one of the amino acid residues of human PD-1 protein, preferably when the ISVD and human PD-1 protein are mixed in PBS1× buffer and crystallized by hanging drop vapor diffusion using 30% PEG3350, 50 mM TRIS, pH 8 at 19° C., preferably wherein the ISVD comprises at least the following amino acid residue: R45 (Kabat numbering);
wherein R45 (Kabat numbering) of the ISVD is capable of forming salt bridges with the amino acid residue E136 of human PD-1 protein, when numbered in accordance with SEQ ID NO: 4703.

20-26. (canceled)

27. The ISVD according to claim 1,
that essentially consists of a heavy chain variable domain sequence that is derived from a conventional four-chain antibody or that essentially consists of a heavy chain variable domain sequence that is derived from a heavy chain antibody;
that essentially consists of a VHH, a humanized VHH, a camelized VH, a domain antibody, a single domain antibody, or a dAb;
which is a humanized ISVD that is chosen from SEQ ID NO: 1-401, 403-488 or from the group consisting of amino acid sequences that have more than 80%, preferably more than 90%, more preferably more than 95%, such as 99% or more amino acid sequence identity with at least one of the amino acid sequences of SEQ ID NOs: 1-488;
which is a humanized ISVD that is chosen from SEQ ID NO: 332, 375, 212, 139, and 238 or from the group consisting of amino acid sequences that have more than 80%, preferably more than 90%, more preferably more than 95%, such as 99% or more amino acid sequence identity with at least one of the amino acid sequences of SEQ ID NOs: 402, 332, 375, 212, 139, 238;
in which the ISVD consists of the amino acid sequence of SEQ ID NO: 402 or 238;
wherein the ISVD specifically binds to human PD-1 with a dissociation constant (K_{D}) of 5×10⁻⁹ to 10⁻¹² moles/litre or less, and preferably 10⁻¹⁰ to 10⁻¹² moles/litre or less and more preferably 5×10⁻¹⁰ to 5×10⁻¹¹ moles/litre, such as 4.3×10⁻¹¹ moles/litre, as determined by Surface Plasmon Resonance;
wherein the ISVD specifically binds to human PD-1 with a kₒₙ-rate of between 10⁵ M⁻¹ s⁻¹ to about 10⁷ M⁻¹ s⁻¹, preferably between 5×10⁵ M⁻¹ s⁻¹ and 10⁷ M⁻¹ s⁻¹, more preferably between 5×10⁵ M⁻¹ s⁻¹ and 5×10⁶ M⁻¹ s⁻¹, such as between 5×10⁵ M⁻¹ s⁻¹ and 1×10⁶ M⁻¹ s⁻¹, such as 3×10⁵ M⁻¹ s⁻¹, as determined by Surface Plasmon Resonance;
wherein the ISVD specifically binds to human PD-1 with a k_{off} rate between 10⁻³ s⁻¹ and 10⁻⁸ s⁻¹, preferably 10⁻³ s⁻¹ and 5×10⁻⁶ s⁻¹, more preferably between 5×10⁻⁴ s⁻¹ and 5×10⁻⁶ s⁻¹, such as between 5×10⁻⁴ s⁻¹ and 5×10⁻⁵ s⁻¹, such as 1.3×10⁻⁵ s⁻¹, as determined by Surface Plasmon Resonance;
wherein the ISVD specifically binds to human and cyno PD-1 and does not bind to CD28, CTLA-4, ICOS, and BTLA or other members of the CD28 receptor family;
wherein the ISVD antagonizes an activity of PD-L1;
wherein the ISVD blocks the interaction of human PD-1 with human PD-L1 with a potency (IC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 10⁻⁹ M, between 10⁻¹⁰ M and 10⁻⁸ M or between 10⁻⁹ M and 10⁻⁸ M, such as 5.11×10⁻⁹ M, for example, as measured in a FACS competition assay on CHO cells expressing human PD-1 with hPD-L1 as a competitor;
wherein the ISVD blocks the interaction of cyno PD-1 with cyno PD-L1 with a potency (IC50 value) of 10⁻⁷ M or lower, of 5×10⁻⁸ M or lower, more preferably of 10⁻⁸ M or lower, or even of 5×10⁻⁹ M or lower, such as between 10⁻¹¹ M and 10⁻⁷ M, between 10⁻¹¹ M and 10⁻⁸ M, between 10⁻¹⁰ M and 10⁻⁷ M, between 10⁻¹⁰ M and 10⁻⁹ M, between 10⁻¹⁰ M and 10⁻⁸ M, between 10⁻⁹ M and 10⁻⁸ M or between 10⁻⁹ M and 10⁻⁷ M, such as 5.76×10⁻⁸ M, for example, as measured in a FACS competition assay on CHO cells expressing cyno PD-1 with hPD-L1 as a competitor;
wherein the ISVD blocks the binding of hPD-1 to hPD-L1 by at least 50%, such as at least 60%, 70%, 80%, 90%, 95%, 99% or even more, as determined by ligand competition, AlphaScreen, or competitive binding assays (such as competition ELISA or competition FACS);
wherein the ISVD induces T-cell activation with a potency (EC50 value) of 10⁻⁸ M or lower, more preferably of 5×10⁻⁹ M or lower, or even of 10⁻⁹ M or lower, such as between 10⁻¹¹ M and 10⁻⁸ M, between 10⁻¹⁰ M and 10⁻⁸ M, between 10⁻¹⁰ M and 10⁻⁹ M or between 10⁻⁹ M and 10⁻⁸ M, such as 2.2×10⁻⁹ M or 2.8×10⁻⁹ M, as determined in a TT assay.

28-40. (canceled)

41. A polypeptide or construct that comprises or essentially consists of one or more ISVDs according to claim 1, and optionally further comprises one or more other groups, residues, moieties or binding units, optionally linked via one or more linkers; optionally
in which said one or more linkers are one or more amino acid sequences;
in which said one or more other groups, residues, moieties or binding units are immunoglobulin sequences;
in which said one or more other groups, residues, moieties or binding units are ISVDs;
in which said one or more other groups, residues, moieties or binding units are chosen from the group consisting of VHHs, humanized VHHs, camelized VHs, domain antibodies, single domain antibodies and dAbs;
which is a multivalent construct;
which is a multispecific construct;
in which said one or more other groups, residues, moieties or binding units provide the polypeptide or construct with increased half-life, compared to the ISVD without the one or more other groups, residues, moieties or binding units;
in which said one or more other groups, residues, moieties or binding units that provide the polypeptide or construct with increased half-life is chosen from the group consisting of a polyethylene glycol molecule (PEG), serum proteins or fragments thereof, binding units that specifically bind to serum proteins, an Fc portion, and small proteins or peptides that specifically bind to serum proteins;
in which said one or more other groups, residues, moieties or binding units that provide the polypeptide or construct with increased half-life is chosen from the group consisting of human serum albumin or fragments thereof;
in which said one or more other groups, residues, moieties or binding units that provides the polypeptide or construct with increased half-life are chosen from the group consisting of binding units that specifically bind to serum albumin (such as human serum albumin) or a serum immunoglobulin (such as IgG);
in which said one or more other groups, residues, moieties or binding units that provides the polypeptide or construct with increased half-life are chosen from the group consisting of VHHs, humanized VHHs, camelized VHs, domain antibodies, single domain antibodies, or dAbs that specifically bind to serum albumin (such as human serum albumin) or a serum immunoglobulin (such as IgG); and/or
in which said one or more other groups, residues, moieties or binding units that provides the polypeptide or construct with increased half-life is an ISVD that specifically binds human serum albumin; optionally wherein said ISVD that specifically binds human serum albumin essentially consists of 4 framework regions (FR1 to FR4, respectively) and 3 complementarity determining regions (CDR1 to CDR3 respectively), in which:
CDR1 (AbM definition) has an amino acid sequence selected from:
a) the amino acid sequence of SEQ ID NO: 2436;
b) amino acid sequences that have at least 80% amino acid identity with the amino acid sequence of SEQ ID NO: 2436; and
c) amino acid sequences that have 3, 2, or 1 amino acid difference with the amino acid sequence of SEQ ID NO: 2436;
and
CDR2 (AbM definition) has an amino acid sequence selected from:
a) the amino acid sequence of SEQ ID NO: 2438;
b) amino acid sequences that have at least 80% amino acid identity with the amino acid sequence of SEQ ID NO: 2438;
c) amino acid sequences that have 3, 2, or 1 amino acid difference with the amino acid sequence of SEQ ID NO: 2438;
and
CDR3 (AbM definition) has an amino acid sequence selected from:
a) the amino acid sequence of SEQ ID NO: 2440;
b) amino acid sequences that have at least 80% amino acid identity with the amino acid sequence of SEQ ID NO: 2440;
c) amino acid sequences that have 3, 2, or 1 amino acid difference with the amino acid sequence of SEQ ID NO: 2440; optionally
wherein CDR1 consists of the amino acid sequence of SEQ ID NO: 2436, CDR2 consists of the amino acid sequence of SEQ ID NO: 2438, and CDR3 consists of the amino acid sequence of SEQ ID NO: 2440;
wherein said ISVD that specifically binds human serum albumin is selected from the group consisting of ALB8 (SEQ ID NO: 2401), ALB23 (SEQ ID NO: 2402), ALBX00001 (SEQ ID NO: 2415) and ALB23002 (SEQ ID NO: 2416), preferably the ISVD binding to human serum albumin is ALB23002 (SEQ ID NO: 2416);
wherein said linker is chosen from the group consisting of SEQ ID NOs: 2418 to 2434;
further comprising a C-terminal extension; and/or
wherein said C-terminal extension is a C-terminal extension (X)ₙ, in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I).

42-60. (canceled)

61. A nucleic acid that encodes an ISVD according to claim 1, optionally wherein the nucleic acid is in the form of a genetic construct, and/or wherein a (non-human) host or host cell that expresses, or that under suitable circumstances is capable of expressing, an ISVD according to claim 1.

62-63. (canceled)

64. A method for producing an ISVD according to claim 1, at least comprising the steps of:
a) expressing, in a suitable (non-human) host cell or host organism or in another suitable expression system, a nucleic acid encoding the ISVD;
optionally followed by:
b) isolating and/or purifying the ISVD; or
a method for producing an ISVD according to claim 1, said method at least comprising the steps of:
a) cultivating and/or maintaining a (non-human) host or host cell under conditions that are such that said (non-human) host or host cell expresses and/or produces at least one ISVD;
optionally followed by:
b) isolating and/or purifying the ISVD.

65. (canceled)

66. A composition comprising at least one ISVD according to claim 1, optionally wherein the composition is a pharmaceutical composition, and/or further comprising at least one pharmaceutically acceptable carrier, diluent or excipient and/or adjuvant, and that optionally comprises one or more further pharmaceutically active polypeptides and/or compounds.

67-72. (canceled)

73. A method for the diagnosis, prevention and/or treatment of at least one disease and/or disorder, comprising the administration, to a subject in need thereof, of a pharmaceutically active amount of an ISVD according to claim 66, optionally wherein
the at least one disease or disorder that is cancer or is associated with PD-1, with its biological or pharmacological activity, and/or with the biological pathways or signalling in which PD-1 is involved.

74-75. (canceled)

76. An immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1 protein, wherein at least one amino acid residue located in at least one of the CDRs is capable of interacting with at least one amino acid residue in at least one, preferably in each, of the following stretches (of the ectodomain) of human PD-1 protein, when numbered in accordance with SEQ ID NO: 4703: N66-E84, and I126-E136, preferably when the ISVD and human PD-1 protein are mixed in PBS1× buffer and crystallized by hanging drop vapor diffusion using 100 mM Na Cacodylate, 300 mM Na Acetate, 10% PEG1000, pH 6.5 at 19° C.;
an immunoglobulin single variable domain (ISVD) specifically binding human PD-1 protein, that essentially consists of 4 framework regions (FR1 to FR4, respectively) and 3 complementarity determining regions (CDR1 to CDR3, respectively), wherein the ISVD comprises an amino acid residue capable of forming salt bridges at at least position 101 (Kabat numbering), and optionally further at position 99 (Kabat numbering), preferably wherein the ISVD comprises at least the following amino acid residue at the following positions: D101, and optionally K99 (Kabat numbering); or
an immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1, wherein the ISVD cross-blocks the binding to human PD-1 protein of a polypeptide with the amino acid sequence of SEQ ID Nos: 714-1871 or wherein the ISVD is cross-blocked from binding to human PD-1 protein by a polypeptide with the amino acid sequence of SEQ ID Nos: 714-1871.

77-78. (canceled)

79. The ISVD according to claim 76, that essentially consists of 4 framework regions (FR1 to FR4, respectively) and 3 complementarity determining regions (CDR1 to CDR3, respectively), in which:
CDR1 (AbM definition) has an amino acid sequence selected from:
a) the amino acid sequence of X₁X₂X₃X₄X₅X₆X₇X₈X₉X₁₀ (SEQ ID NO: 4691); wherein X₁ is selected from G, K, F, A, Y, T, V, H, R, S, P, I, E, L, or D, X₂ is selected from D, Y, R, E, T, L, K, H, V, S, P, Q, I, G, A, or F, X₃ is selected from T, I, A, E, H, G, P, F, L, K, Q, V, R, Y, or S, X₄ is selected from F, K, D, L, T, I, A, E, V, G, H, Y, Q, S, or R, X₅ is selected from S, I, E, A, F, R, V, Y, L, T, Q, K, D, G, or H, X₆ is selected from E, Q, V, T, S, F, K, P, I, G, R, L, H, or A, X₇ is selected from N, R, Q, V, E, Y, H, A, K, L, P, G, F, I, S, or D, X₅ is selected from D, P, A, T, I, R, V, G, Y, L, F, K, S, I, H, or E, X₉ is selected from I, R, H, M, G, Y, Q, E, K, F, T, S, A, D, or P, and X₁₀ is selected from G, A, L, S, R, H, V, P, Q, E, I, D, Y, T or K;
b) amino acid sequences that have at least 80% amino acid identity with the amino acid sequence of X₁X₂X₃X₄X₅X₆X₇X₈X₉X₁₀ (SEQ ID NO: 4691); wherein X₁ is selected from G, K, F, A, Y, T, V, H, R, S, P, I, E, L, or D, X₂ is selected from D, Y, R, E, T, L, K, H, V, S, P, Q, I, G, A, or F, X₃ is selected from T, I, A, E, H, G, P, F, L, K, Q, V, R, Y, or S, X₄ is selected from F, K, D, L, T, I, A, E, V, G, H, Y, Q, S, or R, X₅ is selected from S, I, E, A, F, R, V, Y, L, T, Q, K, D, G, or H, X₆ is selected from E, Q, V, T, S, F, K, P, I, G, R, L, H, or A, X₇ is selected from N, R, Q, V, E, Y, H, A,K, L, P, G, F, I, S, or D, X₈ is selected from D, P, A, T, I, R, V, G, Y, L, S, I, H, or E, X₉ is selected from I, R, H, M, G, Y, Q, E, K, F, T, S, A, D, or P, and X₁₀ is selected from G, A, L, S, R, H, V, P, Q, E, I, D, Y, T, or K;
c) amino acid sequences that have 3, 2, or 1 amino acid difference with the amino acid sequences of X₁X₂X₃X₄X₅X₆X₇X₈X₉X₁₀ (SEQ ID NO: 4691); wherein X₁ is selected from G, K, F, A, Y, T, V, H, R, S, P, I, E, L, or D, X₂ is selected from D, Y, R, E, T, L, K, H, V, S, P, Q, I, G, A, or F, X₃ is selected from T, I, A, E, H, G, P, F, L, K, Q, V, R, Y, or S, X₄ is selected from F, K, D, L, T, I, A, E, V, G, H, Y, Q, S, or R, X₅ is selected from S, I, E, A, F, R, V, Y, L, T, Q, K, D, G, or H, X₆ is selected from E, Q, V, T, S, F, K, P, I, G, R, L, H, or A, X₇ is selected from N, R, Q, V, E, Y, H, A, K, L, P, G, F, I, S, or D, X₈ is selected from D, P, A, T, I, R, V, G, Y, L, S, I, H, or E, X₉ is selected from I, R, H, M, G, Y, Q, E, K, F, T, S, A, D, or P, and X₁₀ is selected from G, A, L, S, R, H, V, P, Q, E, I, D, Y, T, or K;
and
CDR2 (AbM definition) has an amino acid sequence selected from:
a) the amino acid sequence of X₁X₂X₃X₄X₅X₆X₇X₈X₉X₁₀ (SEQ ID NO: 4692); wherein X₁ is selected from A, G, P, F, V, I, R, Q, H, T, S, Y, K, or L, X₂ is selected from I, H, K, G, L, P, F, A, V, T, E, Y, S, Q, D, or R, X₃ is selected from V, K, Y, F, G, Q, A, T, S, P, I, R, D, or L, X₄ is selected from W, P, Y, Q, G, T, D, H, R, K, S, L, I, V, F, or A, X₅ is selected from P, K, Y, F, G, I, L, T, Q, H, E, D, A, S, or V, X₆ is selected from G, Q, E, P, L, F, K, I, S, A, D, R, H, or V, X₇ is selected from G, A, H, F, L, Y, T, P, Q, K, I, R, D, V, or S, X₈ is selected from E, T, I, R, H, Q, D, K, S, P, A, F, Y, V, G, or L, X₉ is selected from T, E, L, Y, F, P, Q, I, R, S, D, K, A, V, or H, and X₁₀ is selected from Y, G, I, L, E, D, H, K, A, F, S, R, P, T, Q;
b) amino acid sequences that have at least 80% amino acid identity with the amino acid sequence of X₁X₂X₃X₄X₅X₆X₇X₈X₉X₁₀ (SEQ ID NO: 4692); wherein X₁ is selected from A, G, P, F, V, I, R, Q, H, A, T, S, Y, K, or L, X₂ is selected from I, H, K, G, L, P, F, A, V, T, E, Y, S, Q, D, or R, X₃ is selected from V, K, Y, F, G, Q, A, T, S, P, I, R, D, or L, X₄ is selected from W, P, Y, Q, G, T, D, H, R, K, S, L, I, V, F, or A, X₅ is selected from P, K, Y, F, G, I, L, T, Q, H, E, D, A, S, or V, X₆ is selected from G, Q, E, P, L, F, K, I, S, A, D, R, H, or V, X₇ is selected from G, A, H, F, L, Y, T, P, Q, K, I, R, D, V, or S, X₈ is selected from E, T, I, R, H, Q, D, K, S, P, A, F, Y, V, G, L, or E, X₉ is selected from T, E, L, Y, F, P, Q, I, S, D, K, A, V, or H, and X₁₀ is selected from Y, G, I, L, E, D, H, K, A, F, S, R, P, T, Q;
c) amino acid sequences that have 3, 2, or 1 amino acid difference with the amino acid sequence of X₁X₂X₃X₄X₅X₆X₇X₈X₉X₁₀ (SEQ ID NO: 4692); wherein X₁ is selected from A, G, P, F, V, I, R, Q, H, A, T, S, Y, K, or L, X₂ is selected from I, H, K, G, L, P, F, A, V, T, E, Y, S, Q, D, or R, X₃ is selected from V, K, Y, F, G, Q, A, T, S, P, I, R, D, or L, X₄ is selected from W, P, Y, Q, G, T, D, H, R, K, S, L, I, V, F, or A, X₅ is selected from P, K, Y, F, G, I, L, T, Q, H, E, D, A, S, or V, X₆ is selected from G, Q, E, P, L, F, K, I, S, A, D, R, H, or V, X₇ is selected from G, A, H, F, L, Y, T, P, Q, K, I, R, D, V, or S, X₈ is selected from E, T, I, R, H, Q, D, K, S, P, A, F, Y, V, G, L, or E, X₉ is selected from T, E, L, Y, F, P, Q, I, S, D, K, A, V, or H, and X₁₀ is selected from Y, G, I, L, E, D, H, K, A, F, S, R, P, T, Q;
and
CDR3 (AbM definition) has an amino acid sequence selected from:
a) the amino acid sequence of X₁X₂X₃X₄X₅X₆X₇X₈X₉X₁₀X₁₁X₁₂X₁₃X₁₄X₁₅X₁₆X₁₇ (SEQ ID NO: 4693); wherein X₁ is selected from K, R, L, Y, T, I, Q, A, F, P, D, H, V, S, or E, X₂ is selected from I, H, T, E, Q, S, L, K, D, G, Y, V, F, R, A, or P, X₃ is selected from F, E, K, A, R, Q, H, T, D, V, L, G, I, Y, or S, X₄ is selected from S, D, G, F, L, K, I, P, Y, V, A, or T, X₅ is selected from K, T, R, P, Y, S, E, I, L, H, D, F, V, A, or G, X₆ is selected from T, K, A, F, S, G, D, P, H, I, Q, Y, L, or V, X₇ is selected from G, F, H, Y, I, L, T, E, P, R, S, Q, D, K, or A, X₈ is selected from T, E, G, V, S, Q, L, H, R, P, A, F, K, I, or Y, X₉ is selected from Y, I, A, Q, G, E, F, D, K, L, S, T, R or V, X₁₀ is selected from S, T, G, L, Q, A, I, K, E, H, D, V, R, P, F, or Y, X₁₁ is selected from A, H, Y, V, R, F, G, L, D, E, T, I, K, P, or S, X₁₂ is selected from S, T, I, D, H, V, R, P, L, K, G, Y, V, E, F, or A, X₁₃ is selected from W, Q, F, H, D, S, K, I, T, E, G, R, L, Y, or A, X₁₄ is selected from E, R, P, I, T, A, F, D, S, H, V, K, L, G, Y, or Q, X₁₅ is selected from Y, Q, L, G, V, P, K, E, I, R, H, D, or F, X₁₆ is selected from D, L, E, F, P, R, T, I, S, Y, G, A, V, K, or H, and X₁₇ is selected from F, S, T, Y, K, R, A, H, G, I, V, D, Q, P, E, or L;
b) amino acid sequences that have at least 80% amino acid identity with the amino acid sequence of X₁X₂X₃X₄X₅X₆X₇X₈X₉X₁₀X₁₁X₁₂X₁₃X₁₄X₁₅X₁₆X₁₇ (SEQ ID NO: 4693); wherein X₁ is selected from K, R, L, Y, T, I, Q, A, F, P, D, H, V, S, or E, X₂ is selected from I, H, T, E, Q, S, L, K, D, G, Y, V, F, R, A, or P, X₃ is selected from F, E, K, A, R, Q, H, T, D, V, L, G, I, Y, or S, X₄ is selected from S, D, G, F, L, K, I, P, Y, V, A, or T, X₅ is selected from K, T, R, P, Y, S, E, I, L, H, D, F, V, A, or G, X₆ is selected from T, K, A, F, S, G, D, P, H, I, Q, Y, L, or V, X₇ is selected from G, F, H, Y, I, L, T, E, P, R, S, Q, D, K, or A, X₈ is selected from T, E, G, V, S, Q, L, H, R, P, A, F, K, I, or Y, X₉ is selected from Y, I, A, Q, G, E, F, D, K, L, S, T, R, or V, X₁₀ is selected from S, T, G, L, Q, A, I, K, E, H, D, V, R, P, F, or Y, X₁₁ is selected from A, H, Y, V, R, F, G, L, D, E, T, I, K, P, or S, X₁₂ is selected from S, T, I, D, H, V, R, P, L, K, G, Y, V, E, F, or A, X₁₃ is selected from W, Q, F, H, D, S, K, I, T, E, G, R, L, Y, or A, X₁₄ is selected from E, R, P, I, T, A, F, D, S, H, V, K, L, G, Y, or Q, X₁₅ is selected from Y, Q, L, G, V, P, K, E, I, R, H, D, or F, X₁₆ is selected from D, L, E, F, P, R, T, I, S, Y, G, A, V, K, or H, and X₁₇ is selected from F, S, T, Y, K, R, A, H, G, I, V, D, Q, P, E, or L;
c) amino acid sequences that have 3, 2, or 1 amino acid difference with the amino acid sequence of X₁X₂X₃X₄X₅X₆X₇X₈X₉X₁₀X₁₁X₁₂X₁₃X₁₄X₁₅X₁₆X₁₇ (SEQ ID NO: 4693);
wherein X₁ is selected from K, R, L, Y, T, I, Q, A, F, P, D, H, V, S, or E, X₂ is selected from I, H, T, E, Q, S, L, K, D, G, Y, V, F, R, A, or P, X₃ is selected from F, E, K, A, R, Q, H, T, D, V, L, G, I, Y, or S, X₄ is selected from S, D, G, F, L, K, I, P, Y, V, A, or T, X₅ is selected from K, T, R, P, Y, S, E, I, L, H, D, F, V, A, or G, X₆ is selected from T, K, A, F, S, G, D, P, H, I, Q, Y, L, or V, X₇ is selected from G, F, H, Y, I, L, T, E, P, R, S, Q, D, K, or A, X₈ is selected from T, E, G, V, S, Q, L, H, R, P, A, F, K, I, or Y, X₉ is selected from Y, I, A, Q, G, E, F, D, K, L, S, T, R, or V, X₁₀ is selected from S, T, G, L, Q, A, I, K, E, H, D, V, R, P, F, or Y, X₁₁ is selected from A, H, Y, V, R, F, G, L, D, E, T, I, K, P, or S, X₁₂ is selected from S, T, I, D, H, V, R, P, L, K, G, Y, V, E, F, or A, X₁₃ is selected from W, Q, F, H, D, S, K, I, T, E, G, R, L, Y, or A, X₁₄ is selected from E, R, P, I, T, A, F, D, S, H, V, K, L, G, Y, or Q, X₁₅ is selected from Y, Q, L, G, V, P, K, E, I, R, H, D, or F, X₁₆ is selected from D, L, E, F, P, R, T, I, S, Y, G, A, V, K, or H, and X₁₇ is selected from F, S, T, Y, K, R, A, H, G, I, V, D, Q, P, E, or L; optionally wherein:
CDR1 (AbM definition) has an amino acid sequence selected from:
a) the amino acid sequence of SEQ ID NO: 1957;
b) amino acid sequences that have at least 80% amino acid identity with the amino acid sequence of SEQ ID NO: 1957; and
c) amino acid sequences that have 3, 2, or 1 amino acid difference with the amino acid sequences of SEQ ID NO: 1957;
and:
CDR2 (AbM definition) has an amino acid sequence selected from:
a) the amino acid sequence of SEQ ID NO: 2127;
b) amino acid sequences that have at least 80% amino acid identity with the amino acid sequence of SEQ ID NO: 2127;
c) amino acid sequences that have 3, 2, or 1 amino acid difference with the amino acid sequence of SEQ ID NO: 2127;
and
CDR3 (AbM definition) has an amino acid sequence selected from:
a) the amino acid sequence of SEQ ID NO: 2337;
b) amino acid sequences that have at least 80% amino acid identity with the amino acid sequence of SEQ ID NO: 2337;
c) amino acid sequences that have 3, 2, or 1 amino acid difference with the amino acid sequence of SEQ ID NO: 2337; or
an ISVD that essentially consists of 4 framework regions (FR1 to FR4, respectively) and 3 complementarity determining regions (CDR1 to CDR3, respectively), wherein
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2096, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2063, CDR3 (AbM definition) consists of the amino acid sequence of SEQ ID NO: 2337; or in which
CDR1 (AbM definition) consists of the amino acid sequence of SEQ ID NO: 2003, CDR2 (AbM definition) consists of the amino acid sequence of SEQ ID NO: 2117, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2117, CDR3 (AbM definition) consists of the amino acid sequence of SEQ ID NO: 2337; or in which
CDR1 (AbM definition) consists of the amino acid sequence of SEQ ID NO: 2003, CDR2 (AbM definition) consists of the amino acid sequence of SEQ ID NO: 2062, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2062, CDR3 (AbM definition) consists of the amino acid sequence of SEQ ID NO: 2337; or in which
CDR1 (AbM definition) consists of the amino acid sequence of SEQ ID NO: 2003, CDR2 (AbM definition) consists of the amino acid sequence of SEQ ID NO: 2097, CDR3 (AbM definition) consists of the amino acid sequence of SEQ ID NO: 2337; or in which
CDR1 (AbM definition) consists of the amino acid sequence of SEQ ID NO: 2003, CDR2 (AbM definition) consists of the amino acid sequence of SEQ ID NO: 2064, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2064, CDR3 (AbM definition) consists of the amino acid sequence of SEQ ID NO: 2337; or in which
CDR1 (AbM definition) consists of the amino acid sequence of SEQ ID NO: 2003, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2003, CDR2 (AbM definition) consists of the amino acid sequence of SEQ ID NO: 2061, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2061, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2097, CDR3 (AbM definition) consists of the amino acid sequence of SEQ ID NO: 2337; or in which
CDR1 (AbM definition) consists of the amino acid sequence of SEQ ID NO: 2003, CDR2 (AbM definition) consists of the amino acid sequence of SEQ ID NO: 2096, CDR3 (AbM definition) consists of the amino acid sequence of SEQ ID NO: 2337; or in which
CDR1 (AbM definition) consists of the amino acid sequence of SEQ ID NO: 2003, CDR2 (AbM definition) consists of the amino acid sequence of SEQ ID NO: 2063, CDR3 (AbM definition) consists of the amino acid sequence of SEQ ID NO: 2337; or in which
CDR1 (AbM definition) consists of the amino acid sequence of SEQ ID NO: 2003, CDR2 (AbM definition) consists of the amino acid sequence of SEQ ID NO: 2064, CDR3 (AbM definition) consists of the amino acid sequence of SEQ ID NO: 2338; or in which
CDR1 (AbM definition) consists of the amino acid sequence of SEQ ID NO: 2003, CDR2 (AbM definition) consists of the amino acid sequence of SEQ ID NO: 2127, CDR3 (AbM definition) consists of the amino acid sequence of SEQ ID NO: 2338; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2338; or in which
CDR1 (AbM definition) consists of the amino acid sequence of SEQ ID NO: 2003, CDR2 (AbM definition) consists of the amino acid sequence of SEQ ID NO: 2061, CDR3 (AbM definition) consists of the amino acid sequence of SEQ ID NO: 2338; or in which
CDR1 (AbM definition) consists of the amino acid sequence of SEQ ID NO: 1957, CDR2 (AbM definition) consists of the amino acid sequence of SEQ ID NO: 2061, CDR3 (AbM definition) consists of the amino acid sequence of SEQ ID NO: 2338; or in which
CDR1 (AbM definition) consists of the amino acid sequence of SEQ ID NO: 2003, CDR2 (AbM definition) consists of the amino acid sequence of SEQ ID NO: 2096, CDR3 (AbM definition) consists of the amino acid sequence of SEQ ID NO: 2338; or in which
CDR1 (AbM definition) consists of the amino acid sequence of SEQ ID NO: 1957, CDR2 (AbM definition) consists of the amino acid sequence of SEQ ID NO: 2096, CDR3 (AbM definition) consists of the amino acid sequence of SEQ ID NO: 2338; or in which
CDR1 (AbM definition) consists of the amino acid sequence of SEQ ID NO: 1957, CDR2 (AbM definition) consists of the amino acid sequence of SEQ ID NO: 2063, CDR3 (AbM definition) consists of the amino acid sequence of SEQ ID NO: 2338; or in which
CDR1 (AbM definition) consists of the amino acid sequence of SEQ ID NO: 2003, CDR2 (AbM definition) consists of the amino acid sequence of SEQ ID NO: 2117, CDR3 (AbM definition) consists of the amino acid sequence of SEQ ID NO: 2338; or in which
CDR1 (AbM definition) consists of the amino acid sequence of SEQ ID NO: 1957, CDR2 (AbM definition) consists of the amino acid sequence of SEQ ID NO: 2117, CDR3 (AbM definition) consists of the amino acid sequence of SEQ ID NO: 2338; or in which
CDR1 (AbM definition) consists of the amino acid sequence of SEQ ID NO: 2003, CDR2 (AbM definition) consists of the amino acid sequence of SEQ ID NO: 2062, CDR3 (AbM definition) consists of the amino acid sequence of SEQ ID NO: 2338; or in which
CDR1 (AbM definition) consists of the amino acid sequence of SEQ ID NO: 1957, CDR2 (AbM definition) consists of the amino acid sequence of SEQ ID NO: 2062, CDR3 (AbM definition) consists of the amino acid sequence of SEQ ID NO: 2338; or in which
CDR1 (AbM definition) consists of the amino acid sequence of SEQ ID NO: 2003, CDR2 (AbM definition) consists of the amino acid sequence of SEQ ID NO: 2097, CDR3 (AbM definition) consists of the amino acid sequence of SEQ ID NO: 2338; or in which
CDR1 (AbM definition) consists of the amino acid sequence of SEQ ID NO: 2003, CDR2 (AbM definition) consists of the amino acid sequence of SEQ ID NO: 2063, CDR3 (AbM definition) consists of the amino acid sequence of SEQ ID NO: 2338; or in which
CDR1 (AbM definition) consists of the amino acid sequence of SEQ ID NO: 1957, CDR2 (AbM definition) consists of the amino acid sequence of SEQ ID NO: 2097, CDR3 (AbM definition) consists of the amino acid sequence of SEQ ID NO: 2338; or in which
CDR1 (AbM definition) consists of the amino acid sequence of SEQ ID NO: 1957, CDR2 (AbM definition) consists of the amino acid sequence of SEQ ID NO: 2064, CDR3 (AbM definition) consists of the amino acid sequence of SEQ ID NO: 2338; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2339; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2340; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2341; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2342; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2343; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2344; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2364; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2365; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2366; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2367; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2368; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2369; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2370; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2371; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2372; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2373; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2374; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2375; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2376; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2377; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2378; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2379; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2380; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2381; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2382; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2383; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2384; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2385; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2386; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2387; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2388; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 1890; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2390; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4044; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3917; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4042; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4002; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3999; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3982; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3833; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4009; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4007; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4005; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3929; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4003; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3813; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3835; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3834; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3847; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3920; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3919; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4000; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4035; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4017; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4021; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4013; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4041; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4032; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4027; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4024; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4036; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3935; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4015; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4019; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4038; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4030; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4039; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4011; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4022; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3900; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3921; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4025; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4031; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3819; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3824; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4045; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3817; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3905; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3818; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4028; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4046; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3918; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3822; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3821; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4047; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3820; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2352; or in which
CDR1 (AbM definition) consists of the amino acid sequence of SEQ ID NO: 1957, CDR2 (AbM definition) consists of the amino acid sequence of SEQ ID NO: 2096, CDR3 (AbM definition) consists of the amino acid sequence of SEQ ID NO: 3812; or in which
CDR1 (AbM definition) consists of the amino acid sequence of SEQ ID NO: 2003, CDR2 (AbM definition) consists of the amino acid sequence of SEQ ID NO: 2061, CDR3 (AbM definition) consists of the amino acid sequence of SEQ ID NO: 3812; or in which
CDR1 (AbM definition) consists of the amino acid sequence of SEQ ID NO: 1957, CDR2 (AbM definition) consists of the amino acid sequence of SEQ ID NO: 2061, CDR3 (AbM definition) consists of the amino acid sequence of SEQ ID NO: 3812; or in which
CDR1 (AbM definition) consists of the amino acid sequence of SEQ ID NO: 2003, CDR2 (AbM definition) consists of the amino acid sequence of SEQ ID NO: 2096, CDR3 (AbM definition) consists of the amino acid sequence of SEQ ID NO: 3812; or in which
CDR1 (AbM definition) consists of the amino acid sequence of SEQ ID NO: 2003, CDR2 (AbM definition) consists of the amino acid sequence of SEQ ID NO: 2063, CDR3 (AbM definition) consists of the amino acid sequence of SEQ ID NO: 3812; or in which
CDR1 (AbM definition) consists of the amino acid sequence of SEQ ID NO: 1957, CDR2 (AbM definition) consists of the amino acid sequence of SEQ ID NO: 2063, CDR3 (AbM definition) consists of the amino acid sequence of SEQ ID NO: 3812; or in which
CDR1 (AbM definition) consists of the amino acid sequence of SEQ ID NO: 1957, CDR2 (AbM definition) consists of the amino acid sequence of SEQ ID NO: 2171, CDR3 (AbM definition) consists of the amino acid sequence of SEQ ID NO: 3940; or in which
CDR1 (AbM definition) consists of the amino acid sequence of SEQ ID NO: 1957, CDR2 (AbM definition) consists of the amino acid sequence of SEQ ID NO: 2058, CDR3 (AbM definition) consists of the amino acid sequence of SEQ ID NO: 2359; or in which
CDR1 (AbM definition) consists of the amino acid sequence of SEQ ID NO: 2003, CDR2 (AbM definition) consists of the amino acid sequence of SEQ ID NO: 2062, CDR3 (AbM definition) consists of the amino acid sequence of SEQ ID NO: 3812; or in which
CDR1 (AbM definition) consists of the amino acid sequence of SEQ ID NO: 1957, CDR2 (AbM definition) consists of the amino acid sequence of SEQ ID NO: 2184, CDR3 (AbM definition) consists of the amino acid sequence of SEQ ID NO: 3940; or in which
CDR1 (AbM definition) consists of the amino acid sequence of SEQ ID NO: 1957, CDR2 (AbM definition) consists of the amino acid sequence of SEQ ID NO: 2171, CDR3 (AbM definition) consists of the amino acid sequence of SEQ ID NO: 2359; or in which
CDR1 (AbM definition) consists of the amino acid sequence of SEQ ID NO: 1957, CDR2 (AbM definition) consists of the amino acid sequence of SEQ ID NO: 2184, CDR3 (AbM definition) consists of the amino acid sequence of SEQ ID NO: 2359; or in which
CDR1 (AbM definition) consists of the amino acid sequence of SEQ ID NO: 1957, CDR2 (AbM definition) consists of the amino acid sequence of SEQ ID NO: 2062, CDR3 (AbM definition) consists of the amino acid sequence of SEQ ID NO: 3812; or in which
CDR1 (AbM definition) consists of the amino acid sequence of SEQ ID NO: 2003, CDR2 (AbM definition) consists of the amino acid sequence of SEQ ID NO: 2117, CDR3 (AbM definition) consists of the amino acid sequence of SEQ ID NO: 3812; or in which
CDR1 (AbM definition) consists of the amino acid sequence of SEQ ID NO: 1957, CDR2 (AbM definition) consists of the amino acid sequence of SEQ ID NO: 2058, CDR3 (AbM definition) consists of the amino acid sequence of SEQ ID NO: 3940; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3812; or in which
CDR1 (AbM definition) consists of the amino acid sequence of SEQ ID NO: 1957, CDR2 (AbM definition) consists of the amino acid sequence of SEQ ID NO: 2064, CDR3 (AbM definition) consists of the amino acid sequence of SEQ ID NO: 3812; or in which
CDR1 (AbM definition) consists of the amino acid sequence of SEQ ID NO: 2003, CDR2 (AbM definition) consists of the amino acid sequence of SEQ ID NO: 2127, CDR3 (AbM definition) consists of the amino acid sequence of SEQ ID NO: 3812; or in which
CDR1 (AbM definition) consists of the amino acid sequence of SEQ ID NO: 2003, CDR2 (AbM definition) consists of the amino acid sequence of SEQ ID NO: 2097, CDR3 (AbM definition) consists of the amino acid sequence of SEQ ID NO: 3812; or in which
CDR1 (AbM definition) consists of the amino acid sequence of SEQ ID NO: 1957, CDR2 (AbM definition) consists of the amino acid sequence of SEQ ID NO: 2117, CDR3 (AbM definition) consists of the amino acid sequence of SEQ ID NO: 3812; or in which
CDR1 (AbM definition) consists of the amino acid sequence of SEQ ID NO: 2003, CDR2 (AbM definition) consists of the amino acid sequence of SEQ ID NO: 2064, CDR3 (AbM definition) consists of the amino acid sequence of SEQ ID NO: 3812; or in which
CDR1 (AbM definition) consists of the amino acid sequence of SEQ ID NO: 1957, CDR2 (AbM definition) consists of the amino acid sequence of SEQ ID NO: 2097, CDR3 (AbM definition) consists of the amino acid sequence of SEQ ID NO: 3812; or in which
CDR1 (AbM definition) consists of the amino acid sequence of SEQ ID NO: 1957, CDR2 (AbM definition) consists of the amino acid sequence of SEQ ID NO: 2171, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2058, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2184, CDR3 (AbM definition) consists of the amino acid sequence of SEQ ID NO: 2337; or in which
CDR1 (AbM definition) consists of the amino acid sequence of SEQ ID NO: 2003, CDR2 (AbM definition) consists of the amino acid sequence of SEQ ID NO: 2183, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3841; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4001; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3998; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3842; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3844; or in which
CDR1 (AbM definition) consists of the amino acid sequence of SEQ ID NO: 1957, CDR2 (AbM definition) consists of the amino acid sequence of SEQ ID NO: 2189, CDR3 (AbM definition) consists of the amino acid sequence of SEQ ID NO: 2337; or in which
CDR1 (AbM definition) consists of the amino acid sequence of SEQ ID NO: 1954, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1961, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1965, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1964, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1958, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2172, CDR3 (AbM definition) consists of the amino acid sequence of SEQ ID NO: 2337; or in which
CDR1 (AbM definition) consists of the amino acid sequence of SEQ ID NO: 1967, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1962, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1963, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1956, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1959, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1955, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1968, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1960, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2182, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2194, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2186, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2054, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2173, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2174, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2191, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2190, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2185, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2055, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2193, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2192, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2195, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2187, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2091, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2053, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2050, CDR3 (AbM definition) consists of the amino acid sequence of SEQ ID NO: 2337; or in which
CDR1 (AbM definition) consists of the amino acid sequence of SEQ ID NO: 1984, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1947, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1987, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1981, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1985, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1988, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1979, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1989, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1973, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1983, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1992, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1991, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1994, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1940, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1953, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2180, CDR3 (AbM definition) consists of the amino acid sequence of SEQ ID NO: 2337; or in which
CDR1 (AbM definition) consists of the amino acid sequence of SEQ ID NO: 1978, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1970, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1982, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1952, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1976, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1972, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1950, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1969, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1951, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1975, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1974, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1948, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1949, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1971, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1986, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1932, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2169, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2059, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2144, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2086, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2087, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2089, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2092, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2167, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2165, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2088, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2077, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2170, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2085, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2090, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2084, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2161, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2083, CDR3 (AbM definition) consists of the amino acid sequence of SEQ ID NO: 2337; or in which
CDR1 (AbM definition) consists of the amino acid sequence of SEQ ID NO: 1995, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2094, CDR3 (AbM definition) consists of the amino acid sequence of SEQ ID NO: 2337; or in which
CDR1 (AbM definition) consists of the amino acid sequence of SEQ ID NO: 1943, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2160, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2159, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2095, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2158, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2157, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2166, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2078, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2163, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2093, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2162, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2156, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2164, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2168, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2072, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2081, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2065, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2056, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2068, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2067, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2179, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2070, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2052, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2181, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2177, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2178, CDR3 (AbM definition) consists of the amino acid sequence of SEQ ID NO: 2337; or in which
CDR1 (AbM definition) consists of the amino acid sequence of SEQ ID NO: 2036, CDR2 (AbM definition) consists of the amino acid sequence of SEQ ID NO: 2175, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2051, CDR3 (AbM definition) consists of the amino acid sequence of SEQ ID NO: 2337; or in which
CDR1 (AbM definition) consists of the amino acid sequence of SEQ ID NO: 1938, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2073, CDR3 (AbM definition) consists of the amino acid sequence of SEQ ID NO: 2337; or in which
CDR1 (AbM definition) consists of the amino acid sequence of SEQ ID NO: 1980, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2176, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2079, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2075, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2080, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2076, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2074, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2082, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2071, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2069, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2060, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2066, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2057, CDR3 (AbM definition) consists of the amino acid sequence of SEQ ID NO: 2337; or in which
CDR1 (AbM definition) consists of the amino acid sequence of SEQ ID NO: 1977, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1945, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2039, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2032, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1908, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2023, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2034, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2029, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2028, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2026, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1905, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2046, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2044, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2045, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2037, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2042, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2043, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2041, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2038, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2035, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1907, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2033, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2040, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1906, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2030, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1927, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2007, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2006, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1925, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1928, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1936, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1931, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2013, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 3883, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2009, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1934, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2008, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1935, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1933, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1926, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1929, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1930, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2005, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1996, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2000, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1946, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1990, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1942, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1941, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1944, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1993, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1939, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2002, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1999, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1997, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2004, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2001, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1998, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2031, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1937, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1915, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1910, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1911, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1914, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1913, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1918, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2027, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2025, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1909, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2036, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2024, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1912, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1917, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1916, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1919, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2021, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1923, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2014, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2018, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2012, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1922, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1924, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2019, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2010, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1920, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2011, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2020, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2015, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2017, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2016, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 2022, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1921, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2148, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2155, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4034; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3868; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3964; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3869; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4006; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4014; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3865; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3867; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4012; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4004; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2361; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2363; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2362; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3932; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2389; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4008; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3963; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4016; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4020; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3934; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4023; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3816; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4018; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 1893; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4010; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4037; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4026; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4029; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4048; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4040; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3968; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4043; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2357; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3975; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2354; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3871; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3879; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3878; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3876; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3875; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2353; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2356; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3977; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3976; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2355; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3866; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3930; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3969; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3967; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3971; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3965; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2360; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3933; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2358; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3873; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3882; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3880; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2359; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3966; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3881; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3874; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3970; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3855; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3864; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3856; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 4033; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3850; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 1891; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3990; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3994; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3996; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3892; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3992; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3993; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3988; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3991; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3987; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3851; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3958; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3997; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3950; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3995; or in which
CDR1 (AbM definition) consists of the amino acid sequence of SEQ ID NO: 1957, CDR2 (AbM definition) consists of the amino acid sequence of SEQ ID NO: 2153, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2149, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3840; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3849; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3848; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3989; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3845; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3838; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3839; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3846; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3843; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3836; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3852; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3837; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3948; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3949; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3953; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3941; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3858; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3863; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3938; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3859; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3861; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3853; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3946; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3862; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3860; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3937; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3857; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3936; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3940; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3956; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3973; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3944; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3960; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3943; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3942; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3961; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3962; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3959; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3945; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3954; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3939; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3951; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3952; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 1892; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3854; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3974; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2351; or in which
CDR1 (AbM definition) consists of the amino acid sequence of SEQ ID NO: 1966, CDR2 (AbM definition) consists of the amino acid sequence of SEQ ID NO: 2127, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2119, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2121, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2115, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2114, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2118, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2120, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2103, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2108, CDR3 (AbM definition) consists of the amino acid sequence of SEQ ID NO: 3813; or in which
CDR1 (AbM definition) consists of the amino acid sequence of SEQ ID NO: 1957, CDR2 (AbM definition) consists of the amino acid sequence of SEQ ID NO: 2129, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2104, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2109, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2110, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2113, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2106, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2116, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2125, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2124, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2111, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2125, CDR3 (AbM definition) consists of the amino acid sequence of SEQ ID NO: 3937; or in which
CDR1 (AbM definition) consists of the amino acid sequence of SEQ ID NO: 1957, CDR2 (AbM definition) consists of the amino acid sequence of SEQ ID NO: 2122, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2126, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2123, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2141, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2112, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2133, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2139, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2132, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2138, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2099, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2134, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2150, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2145, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2147, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2146, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2151, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2154, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2152, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2102, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2140, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2136, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2098, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2107, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2108, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2101, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2128, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2137, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2105, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2130, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2143, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2142, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2131, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2100, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2135, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3981; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3978; or in which
CDR1 (AbM definition) consists of the amino acid sequence of SEQ ID NO: 1957, CDR2 (AbM definition) consists of the amino acid sequence of SEQ ID NO: 2188, CDR3 (AbM definition) consists of the amino acid sequence of SEQ ID NO: 2337; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3986; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3984; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2350; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3980; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2345; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2348; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3979; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2346; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2349; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3985; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3983; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 2347; or in which
CDR1 (AbM definition) consists of the amino acid sequence of SEQ ID NO: 1957, CDR2 (AbM definition) consists of the amino acid sequence of SEQ ID NO: 2127, CDR3 (AbM definition) consists of the amino acid sequence of SEQ ID NO: 3877.

80-81. (canceled)

82. An immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1 protein, wherein at least one amino acid residue located in at least one of the CDRs is capable of interacting with at least one amino acid residue in at least one, preferably in each, of the following stretches (of the ectodomain) of human PD-1 protein, when numbered in accordance with SEQ ID NO: 4703: W32, Y68-M70, K78, and I126-R139, preferably when the ISVD and human PD-1 protein are mixed in PBS1× buffer and crystallized by hanging drop vapor diffusion using 1 M Na Acetate, 1M (NH₄)₂HPO₄, pH 4.5 at 19° C.;
an immunoglobulin single variable domain (ISVD) specifically binding human PD-1 protein, that essentially consists of 4 framework regions (FR1 to FR4, respectively) and 3 complementarity determining regions (CDR1 to CDR3, respectively), wherein the ISVD comprises an amino acid residue capable of forming hydrogen bonds at at least one, preferably at all, of the following positions: 100, 100b (Kabat numbering) and optionally further at position 32, 52, 54, 57, 99 (Kabat numbering, preferably wherein the ISVD comprises at least one, preferably all of the following amino acid residues at the following positions: S100, Y100b (Kabat numbering) and optionally N32, S52, N54, T57, S99 (Kabat numbering);
an immunoglobulin single variable domain (ISVD) specifically binding to human PD-1 protein, that essentially consists of 4 framework regions (FR1 to FR4, respectively) and 3 complementarity determining regions (CDR1 to CDR3, respectively), wherein the ISVD binds to an (discontinuous) epitope on human PD-1, wherein the ISVD cross-blocks the binding to human PD-1 protein of a polypeptide with the amino acid sequence of SEQ ID Nos: 2857-3662 or wherein the ISVD is cross-blocked from binding to human PD-1 protein by a polypeptide with the amino acid sequence of SEQ ID Nos: 2857-3662;
an immunoglobulin single variable domain (ISVD) specifically binding human PD-1, that essentially consists of 4 framework regions (FR1 to FR4, respectively) and 3 complementarity determining regions (CDR1 to CDR3, respectively), in which:
CDR1 (AbM definition) has an amino acid sequence selected from:
a) the amino acid sequence of X₁X₂X₃X₄X₅X₆X₇X₈MA (SEQ ID NO: 4608), wherein X₁ is selected from G, T, D, F, S, R, E, H, I, A, V, or K, X₂ is selected from R, M, D, H, Y, F, V, L, Q, K, P, A, I, or G, wherein X₃ is selected from M, F, R, S, I, D, V, K, T, Y, A, E, or G, X₄ is selected from H, R, S, E, P, G, Q, K, A, D, T, V, I, F, Y, or L, X₅ is selected from S, T, K, G, I, Y, H, F, D, E, V, P, and L, X₆ is selected from I, V, S, K, E, D, Q, R, P, G, T, L, or F, X₇ is selected from N, G, V, E, L, I, Y, Q, T, F, P, or H, and X₈ is selected from A, D, E, I, H, S, V, L, K, G, T, or Y;
b) amino acid sequences that have at least 80% amino acid identity with the amino acid sequence of X₁X₂X₃X₄X₅X₆X₇X₈MA (SEQ ID NO: 4608), wherein X₁ is selected from G, T, D, F, S, R, E, H, I, A, V, or K, X₂ is selected from R, M, D, H, Y, F, V, L, Q, K, P, A, I, or G, wherein X₃ is selected from M, F, R, S, I, D, V, K, T, Y, A, E, or G, X₄ is selected from H, R, S, E, P, G, Q, K, A, D, T, V, I, F, Y, or L, X₅ is selected from S, T, K, G, I, Y, H, F, D, E, V, P, and L, X₆ is selected from I, V, S, K, E, D, Q, R, P, G, T, L, or F, X₇ is selected from N, G, V, E, L, I, Y, Q, T, F, P, or H, and X₈ is selected from A, D, E, E, I, H, S, V, L, K, G, T, or Y; and
c) amino acid sequences that have 3, 2, or 1 amino acid difference with the amino acid sequences of X₁X₂X₃X₄X₅X₆X₇X&MA (SEQ ID NO: 4608), wherein X₁ is selected from G, T, D, F, S, R, E, H, I, A, V, or K, X₂ is selected from R, M, D, H, Y, F, V, L, Q, K, P, A, I, or G, wherein X₃ is selected from M, F, R, S, I, D, V, K, T, Y, A, E, or G, X₄ is selected from H, R, S, E, P, G, Q, K, A, D, T, V, I, F, Y, or L, X₅ is selected from S, T, K, G, I, Y, H, F, D, E, V, P, and L, X₆ is selected from I, V, S, K, E, D, Q, R, P, G, T, L, or F, X₇ is selected from N, G, V, E, L, I, Y, Q, T, F, P, or H, and X₈ is selected from A, D, E, E, I, H, S, V, L, K, G, T, or Y;
and
CDR2 (AbM definition) has an amino acid sequence selected from:
a) the amino acid sequence of X₁X₂X₃X₄X₅X₆X₇X₈X₉X₁₀ (SEQ ID NO: 4609), wherein X₁ is selected from L, V, I, T, F, E, Q, D, R, G, S, A, P, H, or K, X₂ is selected from I, G, Y, A, Q, H, T, L, S, D, R, K, or P, and X₃ is selected from S, G, L, V, K, F, I, T, E, A, D, H, P, Q, or R, X₄ is selected from W, L, K, S, Q, V, D, L, H, T, E, I, A, R, P, Y, G, or F, X₅ is selected from G, L, T, Y, R, V, P, Q, F, A, and S, X₆ is selected from N, D, H, T, A, F, E, S, V, Q, I, L, R, Y, G, X₇ is selected from G, Q, F, S, I, H, R, E, V, P, L, D, T, or A, X₈ is selected from I, Q, T, L, P, V, Y, H, D, R, E, A, G, F, S, or K, X₉ is selected from T, L, D, Q, I, S, G, F, E, A, Y, P, V, or K, and X₁₀ is selected from Y, P, R, T, G, A, S, Q, D, E, L, K, I, F, V, or H;
b) amino acid sequences that have at least 80% amino acid identity with the amino acid sequence of X₁X₂X₃X₄X₅X₆X₇X₈X₉X₁₀ (SEQ ID NO: 4609), wherein X₁ is selected from L, V, I, T, F, E, Q, D, R, G, S, A, P, H, or K, X₂ is selected from I, G, Y, A, Q, H, T, L, S, D, R, K, or P, and X₃ is selected from S, G, L, V, K, F, I, T, E, A, D, H, P, Q, or R, X₄ is selected from W, L, K, S, Q, V, D, L, H, T, E, I, A, R, P, Y, G, or F, X₅ is selected from G, L, T, Y, R, V, P, Q, F, A, and S, X₆ is selected from N, D, H, T, A, F, E, S, V, Q, I, L, R, Y, G, X₇ is selected from G, Q, F, S, I, H, R, E, V, P, L, D, T, or A, X₈ is selected from I, Q, T, L, P, V, Y, H, D, R, E, A, G, F, S, or K, X₉ is selected from T, L, D, Q, I, S, G, F, E, A, Y, P, V, or K, and X₁₀ is selected from Y, P, R, T, G, A, S, Q, D, E, L, K, I, F, V, or H
c) amino acid sequences that have 3, 2, or 1 amino acid difference with the amino acid sequence of X₁X₂X₃X₄X₅X₆X₇X₈X₉X₁₀ (SEQ ID NO: 4609), wherein X₁ is selected from L, V, I, T, F, E, Q, D, R, G, S, A, P, H, or K, X₂ is selected from I, G, Y, A, Q, H, T, L, S, D, R, K, or P, and X₃ is selected from S, G, L, V, K, F, I, T, E, A, D, H, P, Q, or R, X₄ is selected from W, L, K, S, Q, V, D, L, H, T, E, I, A, R, P, Y, G, or F, X₅ is selected from G, L, T, Y, R, V, P, Q, F, A, and S, X₆ is selected from N, D, H, T, A, F, E, S, V, Q, I, L, R, Y, G, X₇ is selected from G, Q, F, S, I, H, R, E, V, P, L, D, T, or A, X₈ is selected from I, Q, T, L, P, V, Y, H, D, R, E, A, G, F, S, or K, X₉ is selected from T, L, D, Q, I, S, G, F, E, A, Y, P, V, or K, and X₁₀ is selected from Y, P, R, T, G, A, S, Q, D, E, L, K, I, F, V, or H;
and
CDR3 (AbM definition) has an amino acid sequence selected from:
a) the amino acid sequence of X₁X₂X₃X₄X₅X₆X₇X₈X₉X₁₀ (SEQ ID NO: 4610), wherein X₁ is selected from E, P, D, A, K, I, Y, H, R, F, S, T, V, G, or Q, X₂ is selected S, F, A, Y, E, I, D, K, G, R, P, Q, T, H, or L, and X₃ is selected Y, D, F, E, R, K, I, T, A, Q, S, V, H, G, or P, X₄ is selected from G, L, I, H, S, V, R, P, E, A, K, Y, T, F, A, D, H, or R, X₅ is selected from S, D, K, Y, A, L, I, P, V, Q, E, R, T, G, or F, X₆ is selected from S, T, K, P, H, I, G, V, Q, R, D, L, or E, X₇ is selected from W, C, P, K, Y, V, F, A, I, T, L, H, G, Q, R, D, or E, X₈ is selected from Y, F, Q, P, K, T, R, D, I, E, L, H, V, A, G, or S, and X₉ is selected from D, N, A, S, L, K, T, F, I, G, Y, R, V, E, or H, and X₁₀ is selected from S, D, V, H, A, L, E, T, Y, K, F, I, Q, R, G, or P;
b) amino acid sequences that have at least 80% amino acid identity with the amino acid sequence of X₁X₂X₃X₄X₅X₆X₇X₈X₉X₁₀ (SEQ ID NO: 4610), wherein X₁ is selected from E, P, D, A, K, I, Y, H, R, F, S, T, V, G, or Q, X₂ is selected S, F, A, Y, E, I, D, K, G, R,P, Q, T, H, or L, and X₃ is selected Y, D, F, E, R, K, I, T, A, Q, S, V, H, G, or P, X₄ is selected from G, L, I, H, S, V, R, P, E, A, K, Y, T, F, A, D, H, or R, X₅ is selected from S, D, K, Y, A, L, I, P, V, Q, E, R, T, G, or F, X₆ is selected from S, T, K, P, H, I, G, V, Q, R, D, L, or E, X₇ is selected from W, C, P, K, Y, V, F, A, I, T, L, H, G, Q, R, D, or E, X₈ is selected from Y, F, Q, P, K, T, R, D, I, E, L, H, V, A, G, or S, and X₉ is selected from D, N, A, S, L, K, T, F, I, G, Y, R, V, E, or H, and X₁₀ is selected from S, D, V, H, A, L, E, T, Y, K, F, I, Q, R, G, or P;
c) amino acid sequences that have 3, 2, or 1 amino acid difference with the amino acid sequence of X₁X₂X₃X₄X₅X₆X₇X₈X₉X₁₀ (SEQ ID NO: 4610), wherein X₁ is selected from E, P, D, A, K, I, Y, H, R, F, S, T, V, G, or Q, X₂ is selected S, F, A, Y, E, I, D, K, G, R,P, Q, T, H, or L, and X₃ is selected Y, D, F, E, R, K, I, T, A, Q, S, V, H, G, or P, X₄ is selected from G, L, I, H, S, V, R, P, E, A, K, Y, T, F, A, D, H, or R, X₅ is selected from S, D, K, Y, A, L, I, P, V, Q, E, R, T, G, or F, X₆ is selected from S, T, K, P, H, I, G, V, Q, R, D, L, or E, X₇ is selected from W, C, P, K, Y, V, F, A, I, T, L, H, G, Q, R, D, or E, X₈ is selected from Y, F, Q, P, K, T, R, D, I, E, L, H, V, A, G, or S, and X₉ is selected from D, N, A, S, L, K, T, F, I, G, Y, R, V, E, or H, and X₁₀ is selected from S, D, V, H, A, L, E, T, Y, K, F, I, Q, R, G, or P;
an immunoglobulin single variable domain (ISVD) specifically binding human PD-1, that essentially consists of 4 framework regions (FR1 to FR4, respectively) and 3 complementarity determining regions (CDR1 to CDR3, respectively), in which:
CDR1 (AbM definition) has an amino acid sequence selected from:
a) the amino acid sequence of GRXHSINAMA (SEQ ID NO: 4704), wherein X is selected from M, V and T;
b) amino acid sequences that have at least 80% amino acid identity with the amino acid sequence of GRXHSINAMA (SEQ ID NO: 4704), wherein X is selected from M, V and T;
c) amino acid sequences that have 3, 2, or 1 amino acid difference with the amino acid sequences of GRXHSINAMA (SEQ ID NO: 4704), wherein X is selected from M, V and T;
and
CDR2 (AbM definition) has an amino acid sequence selected from:
a) the amino acid sequence of LISWGXGITY (SEQ ID NO: 4705), wherein X is selected from N and F;
b) amino acid sequences that have at least 80% amino acid identity with the amino acid sequence of LISWGXGITY (SEQ ID NO: 4705), wherein X is selected from N and F;
c) amino acid sequences that have 3, 2, or 1 amino acid difference with the amino acid sequence of LISWGXGITY (SEQ ID NO: 4705), wherein X is selected from N and F;
and
CDR3 (AbM definition) has an amino acid sequence selected from:
a) the amino acid sequence of ESYGXSWYDS (SEQ ID NO: 4706); wherein X is selected from S and A;
b) amino acid sequences that have at least 80% amino acid identity with the amino acid sequence of ESYGXSWYDS (SEQ ID NO: 4706); wherein X is selected from S and A;
c) amino acid sequences that have 3, 2, or 1 amino acid difference with the amino acid sequence of ESYGXSWYDS (SEQ ID NO: 4706); wherein X is selected from S and A, optionally wherein
CDR1 (AbM definition) has an amino acid sequence selected from:
a) the amino acid sequence of GRMHSINAMA (SEQ ID NO: 2498);
b) amino acid sequences that have at least 80% amino acid identity with the amino acid sequence of GRMHSINAMA (SEQ ID NO: 2498); and
c) amino acid sequences that have 3, 2, or 1 amino acid difference with the amino acid sequences of GRMHSINAMA (SEQ ID NO: 2498);
and:
CDR2 (AbM definition) has an amino acid sequence selected from:
a) the amino acid sequence of LISWGNGITY (SEQ ID NO: 2451);
b) amino acid sequences that have at least 80% amino acid identity with the amino acid sequence of LISWGNGITY (SEQ ID NO: 2451);
c) amino acid sequences that have 3, 2, or 1 amino acid difference with the amino acid sequence of LISWGNGITY (SEQ ID NO: 2451);
and:
CDR3 (AbM definition) has an amino acid sequence selected from:
a) the amino acid sequence of ESYGSSWYDS (SEQ ID NOs: 2804);
b) amino acid sequences that have at least 80% amino acid identity with the amino acid sequence of ESYGSSWYDS (SEQ ID NOs: 2804);
c) amino acid sequences that have 3, 2, or 1 amino acid difference with the amino acid sequence of ESYGSSWYDS (SEQ ID NOs: 2804), optionally wherein
CDR1 (AbM definition) has an amino acid sequence selected from:
a) the amino acid sequence of GR VHSINAMA (SEQ ID NO: 2552);
b) amino acid sequences that have at least 80% amino acid identity with the amino acid sequence of GR VHSINAMA (SEQ ID NO: 2552); and
c) amino acid sequences that have 3, 2, or 1 amino acid difference with the amino acid sequences of GR VHSINAMA (SEQ ID NO: 2552);
and
CDR2 (AbM definition) has an amino acid sequence selected from:
a) the amino acid sequence of LISWGFGITY (SEQ ID NO: 2652);
b) amino acid sequences that have at least 80% amino acid identity with the amino acid sequence of LISWGFGITY (SEQ ID NO: 2652);
c) amino acid sequences that have 3, 2, or 1 amino acid difference with the amino acid sequence of LISWGFGITY (SEQ ID NO: 2652);
and
CDR3 (AbM definition) has an amino acid sequence selected from:
a) the amino acid sequence of ESYGASWYDS (SEQ ID NOs: 2741);
b) amino acid sequences that have at least 80% amino acid identity with the amino acid sequence of ESYGASWYDS (SEQ ID NOs: 2741);
c) amino acid sequences that have 3, 2, or 1 amino acid difference with the amino acid sequence of ESYGASWYDS (SEQ ID NOs: 2741); or
an ISVD that essentially consists of 4 framework regions (FR1 to FR4, respectively) and 3 complementarity determining regions (CDR1 to CDR3, respectively), wherein
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2453, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2456, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2559, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 3810, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2455, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2556, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2560, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2557, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2450, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2561, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2454, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2558, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2458, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2460, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2553, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2459, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2554, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2464, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2466, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2462, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2465, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2457, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2463, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2461, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2470, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2549, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2550, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2471, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2468, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2552, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2472, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2551, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2555, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2467, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2469, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2473, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2542, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2547, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2476, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2543, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2480, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2544, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2540, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2477, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2475, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2548, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2545, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2538, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2474, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2546, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2541, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2535, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2486, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2482, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2484, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2537, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2483, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2481, CDR2 (AbM definition) consists of the amino acid sequence of SEQ ID NO: 2451, CDR3 (AbM definition) consists of the amino acid sequence of SEQ ID NO: 2815; or in which
CDR1 (AbM definition) consists of the amino acid sequence of SEQ ID NO: 2536, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2478, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2479, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2539, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2481, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2487, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2485, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2534, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2528, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2490, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2489, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2529, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2530, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2531, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2492, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2533, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2532, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2488, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2494, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2525, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2491, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2495, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2526, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2524, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2527, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2493, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2521, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2496, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2497, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2512, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2511, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2516, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2515, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2520, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2605, CDR3 (AbM definition) consists of the amino acid sequence of SEQ ID NO: 2804; or in which
CDR1 (AbM definition) consists of the amino acid sequence of SEQ ID NO: 2522, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2517, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2518, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2514, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2519, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2523, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2499, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2502, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2503, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2504, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2500, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2505, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2509, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2508, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2510, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2501, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2513, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2507, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2506, CDR2 (AbM definition) consists of the amino acid sequence of SEQ ID NO: 2451, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2698, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2569, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2699, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2564, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2562, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2705, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2695, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2697, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2566, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2700, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2563, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2706, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2565, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2570, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2572, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2701, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2571, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2619, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2575, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2704, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2694, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2702, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2568, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2703, CDR3 (AbM definition) consists of the amino acid sequence of SEQ ID NO: 2804; or in which
CDR1 (AbM definition) consists of the amino acid sequence of SEQ ID NO: 2533, CDR2 (AbM definition) consists of the amino acid sequence of SEQ ID NO: 2674, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2596, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2577, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2587, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2583, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2693, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2580, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2579, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2581, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2688, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2578, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2574, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2573, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2582, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2585, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2586, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2593, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2598, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2589, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2588, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2689, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2594, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2600, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2590, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2609, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2591, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2601, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2597, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2595, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2592, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2603, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2602, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2681, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2686, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2690, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2685, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2687, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2683, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2682, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2604, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2599, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2684, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2610, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2678, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2606, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2652, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2651, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2677, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2679, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2672, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2613, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2611, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2676, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2680, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2669, CDR3 (AbM definition) consists of the amino acid sequence of SEQ ID NO: 2804; or in which
CDR1 (AbM definition) consists of the amino acid sequence of SEQ ID NO: 2509, CDR2 (AbM definition) consists of the amino acid sequence of SEQ ID NO: 2612, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2614, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2671, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2663, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2664, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2668, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2615, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2673, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2670, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2667, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2616, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2675, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2617, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2661, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2659, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2658, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2657, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2666, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2665, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2625, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2654, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2662, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2622, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2618, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2620, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2621, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2660, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2656, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2634, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2626, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2631, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2632, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2633, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2627, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2628, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2623, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2624, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2655, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2630, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2653, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2629, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2642, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2643, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2647, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2636, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2635, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2637, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2648, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2644, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2638, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2639, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2645, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2646, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2641, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2640, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2649, CDR3 (AbM definition) consists of the amino acid sequence of SEQ ID NO: 2804; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2742; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2850; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2707; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2708; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2849; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2848; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2856; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2847; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2852; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2845; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2853; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2854; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2855; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2846; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2851; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2710; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2709; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2844; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2711; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2715; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2712; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2716; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2714; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2718; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2720; or in which
CDR1 (AbM definition) consists of the amino acid sequence of SEQ ID NO: 2498, CDR2 (AbM definition) consists of the amino acid sequence of SEQ ID NO: 2584, CDR3 (AbM definition) consists of the amino acid sequence of SEQ ID NO: 2844; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2719; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2843; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2713; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2717; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2722; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2725; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2723; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2732; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2728; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2726; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2731; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2721; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2729; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2730; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2734; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2727; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2724; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2733; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2833; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2831; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2830; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2839; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2841; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2838; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2834; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2738; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2735; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2832; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2842; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2840; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2739; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2736; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2737; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2771; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2769; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2821; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2775; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2765; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2762; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2768; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2774; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2770; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2767; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2766; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2772; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2773; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2763; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2764; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2779; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2786; or in which
CDR1 (AbM definition) consists of the amino acid sequence of SEQ ID NO: 2498, CDR2 (AbM definition) consists of the amino acid sequence of SEQ ID NO: 2671, CDR3 (AbM definition) consists of the amino acid sequence of SEQ ID NO: 2785; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2783; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2789; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2787; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2777; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2782; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2778; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2784; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2785; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2781; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2790; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2776; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2780; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2788; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2791; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2817; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2814; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2813; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2818; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2809; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2812; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2810; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2820; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2816; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2819; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2808; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2811; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2793; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2806; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2797; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2792; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2800; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2794; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2805; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2807; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2799; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2795; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2798; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2802; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2803; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2796; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2801; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2747; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2826; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2741; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2748; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2746; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2749; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2825; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2750; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2743; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2751; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2824; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2745; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2744; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2822; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2757; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2759; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2755; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2756; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2754; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2823; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2760; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2761; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2752; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2758; or in which
CDR1 (AbM definition) consists of the amino acid sequence of SEQ ID NO: 2498, CDR2 (AbM definition) consists of the amino acid sequence of SEQ ID NO: 2451, CDR3 (AbM definition) consists of the amino acid sequence of SEQ ID NO: 2753; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2804; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2804; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2804; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2804; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2830; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2830; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2830; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2830; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2838; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2838; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2838; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2838; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2741; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2741; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2741; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2741; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2828; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2828; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2828; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2828; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2836; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2836; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2836; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2836; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2785; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2785; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2785; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2785; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2829; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2829; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2829; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2829; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2837; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2837; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2837; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2837; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2740; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2740; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2740; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2740; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2827; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2827; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2827; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2827; or in which
CDR1 (AbM definition) consists of the amino acid sequence of SEQ ID NO: 2551, CDR2 (AbM definition) consists of the amino acid sequence of SEQ ID NO: 2651, CDR3 (AbM definition) consists of the amino acid sequence of SEQ ID NO: 2835; or in which
CDR1 (AbM definition) consists of the amino acid sequence of SEQ ID NO: 2552, CDR2 (AbM definition) consists of the amino acid sequence of SEQ ID NO: 2651, CDR3 (AbM definition) consists of the amino acid sequence of SEQ ID NO: 2835; or in which
CDR1 (AbM definition) consists of the amino acid sequence of SEQ ID NO: 2551, CDR2 (AbM definition) consists of the amino acid sequence of SEQ ID NO: 2652, CDR3 (AbM definition) consists of the amino acid sequence of SEQ ID NO: 2835; or in which
CDR1 (AbM definition) consists of the amino acid sequence of SEQ ID NO: 2552, CDR2 (AbM definition) consists of the amino acid sequence of SEQ ID NO: 2652, CDR3 (AbM definition) consists of the amino acid sequence of SEQ ID NO: 2835; or in which
CDR1 (AbM definition) consists of the amino acid sequence of SEQ ID NO: 2551, CDR2 (AbM definition) consists of the amino acid sequence of SEQ ID NO: 2608, CDR3 (AbM definition) consists of the amino acid sequence of SEQ ID NO: 2804; or in which
CDR1 (AbM definition) consists of the amino acid sequence of SEQ ID NO: 2551, CDR2 (AbM definition) consists of the amino acid sequence of SEQ ID NO: 2576, CDR3 (AbM definition) consists of the amino acid sequence of SEQ ID NO: 2804; or in which
CDR1 (AbM definition) consists of the amino acid sequence of SEQ ID NO: 2551, CDR2 (AbM definition) consists of the amino acid sequence of SEQ ID NO: 2691, CDR3 (AbM definition) consists of the amino acid sequence of SEQ ID NO: 2804; or in which
CDR1 (AbM definition) consists of the amino acid sequence of SEQ ID NO: 2552, CDR2 (AbM definition) consists of the amino acid sequence of SEQ ID NO: 2691, CDR3 (AbM definition) consists of the amino acid sequence of SEQ ID NO: 2741; or in which
CDR1 (AbM definition) consists of the amino acid sequence of SEQ ID NO: 2552, CDR2 (AbM definition) consists of the amino acid sequence of SEQ ID NO: 2567, CDR3 (AbM definition) consists of the amino acid sequence of SEQ ID NO: 2741; or in which
CDR1 (AbM definition) consists of the amino acid sequence of SEQ ID NO: 2552, CDR2 (AbM definition) consists of the amino acid sequence of SEQ ID NO: 2576, CDR3 (AbM definition) consists of the amino acid sequence of SEQ ID NO: 2741; or in which
CDR1 (AbM definition) consists of the amino acid sequence of SEQ ID NO: 2552, CDR2 (AbM definition) consists of the amino acid sequence of SEQ ID NO: 2696, CDR3 (AbM definition) consists of the amino acid sequence of SEQ ID NO: 2741; or in which
CDR1 (AbM definition) consists of the amino acid sequence of SEQ ID NO: 2551, CDR2 (AbM definition) consists of the amino acid sequence of SEQ ID NO: 2567, CDR3 (AbM definition) consists of the amino acid sequence of SEQ ID NO: 2804; or in which
CDR1 (AbM definition) consists of the amino acid sequence of SEQ ID NO: 2551, CDR2 (AbM definition) consists of the amino acid sequence of SEQ ID NO: 2696, CDR3 (AbM definition) consists of the amino acid sequence of SEQ ID NO: 2804; or in which
CDR1 (AbM definition) consists of the amino acid sequence of SEQ ID NO: 2552, CDR2 (AbM definition) consists of the amino acid sequence of SEQ ID NO: 2678, CDR3 (AbM definition) consists of the amino acid sequence of SEQ ID NO: 2741; or in which
CDR1 (AbM definition) consists of the amino acid sequence of SEQ ID NO: 2552, CDR2 (AbM definition) consists of the amino acid sequence of SEQ ID NO: 2677, CDR3 (AbM definition) consists of the amino acid sequence of SEQ ID NO: 2741; or in which
CDR1 (AbM definition) consists of the amino acid sequence of SEQ ID NO: 2551, CDR2 (AbM definition) consists of the amino acid sequence of SEQ ID NO: 2678, CDR3 (AbM definition) consists of the amino acid sequence of SEQ ID NO: 2741; or in which
CDR1 (AbM definition) consists of the amino acid sequence of SEQ ID NO: 2551, CDR2 (AbM definition) consists of the amino acid sequence of SEQ ID NO: 2677, CDR3 (AbM definition) consists of the amino acid sequence of SEQ ID NO: 2741; or in which
CDR1 (AbM definition) consists of the amino acid sequence of SEQ ID NO: 2551, CDR2 (AbM definition) consists of the amino acid sequence of SEQ ID NO: 2678, CDR3 (AbM definition) consists of the amino acid sequence of SEQ ID NO: 2804; or in which
CDR1 (AbM definition) consists of the amino acid sequence of SEQ ID NO: 2551, CDR2 (AbM definition) consists of the amino acid sequence of SEQ ID NO: 2677, CDR3 (AbM definition) consists of the amino acid sequence of SEQ ID NO: 2804.

83-89. (canceled)

90. An immunoglobulin single variable domain (ISVD) specifically binding human PD-1, that essentially consists of 4 framework regions (FR1 to FR4, respectively) and 3 complementarity determining regions (CDR1 to CDR3, respectively), in which:
CDR1 (AbM definition) has an amino acid sequence selected from:
a) the amino acid sequence of GFTLX₁X₂X₃X₄X₅X₆ (SEQ ID NO: 4696), wherein X₁ is selected from Y, F, T, Q, A, S, R, P, D, G, L, E, K, H, or D, X₂ is selected from D, R, T, I, Q, F, H, S, L, V, K, E, P, G, A, or Y, X₃ is selected from H, G, Y, R, D, E, V, P, Q, S, A, K, I, F, T, or L, X₄ is selected from A, L, S, D, R, K, H, Q, T, V, Y, P, I, F, or G, X₅ is selected from V and F, and X₆ is selected from G, Q, E, H, K, P, Y, D, I, T, A, S, R, V, or C;
b) amino acid sequences that have at least 80% amino acid identity with the amino acid sequence of GFTLX₁X₂X₃X₄X₅X₆ (SEQ ID NO: 4696), wherein X₁ is selected from Y, F, T, Q, A, S, R, P, D, G, L, E, K, H, or D, X₂ is selected from D, R, T, I, Q, F, H, S, L, V, K, E, P, G, A, or Y, X₃ is selected from H, G, Y, R, D, E, V, P, Q, S, A, K, I, F, T, or L, X₄ is selected from A, L, S, D, R, K, H, Q, T, V, Y, P, I, F, or G, X₅ is selected from V and F, and X₆ is selected from G, Q, E, H, K, P, Y, D, I, T, A, S, R, V, or C;
c) amino acid sequences that have 3, 2, or 1 amino acid difference with the amino acid sequences of GFTLX₁X₂X₃X₄X₅X₆ (SEQ ID NO: 4696), wherein X₁ is selected from Y, F, T, Q, A, S, R, P, D, G, L, E, K, H, or D, X₂ is selected from D, R, T, I, Q, F, H, S, L, V, K, E, P, G, A, or Y, X₃ is selected from H, G, Y, R, D, E, V, P, Q, S, A, K, I, F, T, or L, X₄ is selected from A, L, S, D, R, K, H, Q, T, V, Y, P, I, F, or G, X₅ is selected from V and F, and X₆ is selected from G, Q, E, H, K, P, Y, D, I, T, A, S, R, V, or C;
and:
CDR2 (AbM definition) has an amino acid sequence selected from:
a) the amino acid sequence of X₁X₂SSSGX₃HEX₄ (SEQ ID NO: 4697), wherein X₁ is selected from V, E, or D, X₂ is selected from I, P, T, S, R, G, H, or K, X₃ is selected from A, R, H, E, K, T, or P, and X₄ is selected from W, F, E, P, D, H, Y, L, R, V, K, G, S, A, or I;
b) amino acid sequences that have at least 80% amino acid identity with the amino acid sequence of X₁X₂SSSGX₃HEX₄ (SEQ ID NO: 4697), wherein X₁ is selected from V, E, or D, X₂ is selected from I, P, T, S, R, G, H, or K, X₃ is selected from A, R, H, E, K, T, or P, and X₄ is selected from W, F, E, P, D, H, Y, L, R, V, K, G, S, A, or I;
c) amino acid sequences that have 3, 2, or 1 amino acid difference with the amino acid sequence of X₁X₂SSSGX₃HEX₄ (SEQ ID NO: 4697), wherein X₁ is selected from V, E, or D, X₂ is selected from I, P, T, S, R, G, H, or K, X₃ is selected from A, R, H, E, K, T, or P, and X₄ is selected from W, F, E, P, D, H, Y, L, R, V, K, G, S, A, or I;
and:
CDR3 (AbM definition) has an amino acid sequence selected from:
a) the amino acid sequence of GX₁X₂X₃YSX₄X₅X₆SEY (SEQ ID NO: 4698); wherein X₁ is selected from G or R, X₂ is selected from M, E, Q, F, V, G, A, I, D, Y, R, K, S, T, H, or P, X₃ is selected from W, L, T, S, P, F, V, I, R, L, Q, E, K, G, D, Y, H, or A, X₄ is selected from D, G, H, K, P, R, or A, X₅ is selected from D, A, V, S, E, I, H, Y, L, G, P, K, Q, R, T, or F, and X₆ is selected from G, F, S, H, Y, I, R, K, L, T, P, V, D, E, Q, or A;
b) amino acid sequences that have at least 80% amino acid identity with the amino acid sequence of GX₁X₂X₃YSX₄X₅X₆SEY (SEQ ID NO: 4698); wherein X₁ is selected from G or R, X₂ is selected from M, E, Q, F, V, G, A, I, D, Y, R, K, S, T, H, or P, X₃ is selected from W, L, T, S, P, F, V, I, R, L, Q, E, K, G, D, Y, H, or A, X₄ is selected from D, G, H, K, P, R, or A, X₅ is selected from D, A, V, S, E, I, H, Y, L, G, P, K, Q, R, T, or F, and X₆ is selected from G, F, S, H, Y, I, R, K, L, T, P, V, D, E, Q, or A;
c) amino acid sequences that have 3, 2, or 1 amino acid difference with the amino acid sequence of GX₁X₂X₃YSX₄X₅X₆SEY (SEQ ID NO: 4698); wherein X₁ is selected from G or R, X₂ is selected from M, E, Q, F, V, G, A, I, D, Y, R, K, S, T, H, or P, X₃ is selected from W, L, T, S, P, F, V, I, R, L, Q, E, K, G, D, Y, H, or A, X₄ is selected from D, G, H, K, P, R, or A, X₅ is selected from D, A, V, S, E, I, H, Y, L, G, P, K, Q, R, T, or F, and X₆ is selected from G, F, S, H, Y, I, R, K, L, T, P, V, D, E, Q, or A;
an immunoglobulin single variable domain (ISVD) specifically binding human PD-1, that essentially consists of 4 framework regions (FR1 to FR4, respectively) and 3 complementarity determining regions (CDR1 to CDR3, respectively), in which:
CDR1 (AbM definition) has an amino acid sequence selected from:
a) the amino acid sequence of GFTLYDHAXG (SEQ ID NO: 4611), wherein X is selected from V or F;
b) amino acid sequences that have at least 80% amino acid identity with the amino acid sequence of GFTLYDHAXG (SEQ ID NO: 4611), wherein X is selected from V or F;
c) amino acid sequences that have 3, 2, or 1 amino acid difference with the amino acid sequences of GFTLYDHAXG (SEQ ID NO: 4611), wherein X is selected from V or F;
and
CDR2 (AbM definition) has an amino acid sequence selected from:
a) the amino acid sequence of VISSSGAHEW (SEQ ID NO: 4481);
b) amino acid sequences that have at least 80% amino acid identity with the amino acid sequence of VISSSGAHEW (SEQ ID NO: 4481);
c) amino acid sequences that have 3, 2, or 1 amino acid difference with the amino acid sequence of VISSSGAHEW (SEQ ID NO: 4481);
and
CDR3 (AbM definition) has an amino acid sequence selected from:
a) the amino acid sequence of GGMWYSDXGSEY (SEQ ID NO: 4612); wherein X is selected from D or E;
b) amino acid sequences that have at least 80% amino acid identity with the amino acid sequence of GGMWYSDXGSEY (SEQ ID NO: 4612); wherein X is selected from D or E;
c) amino acid sequences that have 3, 2, or 1 amino acid difference with the amino acid sequence of GGMWYSDXGSEY (SEQ ID NO: 4612); wherein X is selected from D or E, optionally wherein:
CDR1 (AbM definition) has an amino acid sequence selected from:
a) the amino acid sequence of GFTLYDHAVG (SEQ ID NO: 4412);
b) amino acid sequences that have at least 80% amino acid identity with the amino acid sequence of SEQ ID NO: GFTLYDHAVG (SEQ ID NO: 4412); and
c) amino acid sequences that have 3, 2, or 1 amino acid difference with the amino acid sequences of SEQ ID NO: GFTLYDHAVG (SEQ ID NO: 4412);
and
CDR2 (AbM definition) has an amino acid sequence selected from:
a) the amino acid sequence of VISSSGAHEW (SEQ ID NO: 4481);
b) amino acid sequences that have at least 80% amino acid identity with the amino acid sequence VISSSGAHEW (SEQ ID NO: 4481);
c) amino acid sequences that have 3, 2, or 1 amino acid difference with the amino acid sequence of VISSSGAHEW (SEQ ID NO: 4481);
and
CDR3 (AbM definition) has an amino acid sequence selected from:
a) the amino acid sequence of GGMWYSDDGSEY (SEQ ID NO: 4560);
b) amino acid sequences that have at least 80% amino acid identity with the amino acid sequence of SEQ ID NO: GGMWYSDDGSEY (SEQ ID NO: 4560);
c) amino acid sequences that have 3, 2, or 1 amino acid difference with the amino acid sequence of SEQ ID NO: GGMWYSDDGSEY (SEQ ID NO: 4560); or
an ISVD that essentially consists of 4 framework regions (FR1 to FR4, respectively) and 3 complementarity determining regions (CDR1 to CDR3, respectively), wherein
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4524; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4528; or in which
CDR1 (AbM definition) consists of the amino acid sequence of SEQ ID NO: 4412, CDR2 (AbM definition) consists of the amino acid sequence of SEQ ID NO: 4470, CDR3 (AbM definition) consists of the amino acid sequence of SEQ ID NO: 4528; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4527; or in which
CDR1 (AbM definition) consists of the amino acid sequence of SEQ ID NO: 4412, CDR2 (AbM definition) consists of the amino acid sequence of SEQ ID NO: 4470, CDR3 (AbM definition) consists of the amino acid sequence of SEQ ID NO: 4527; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4524; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4596; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4528; or in which
CDR1 (AbM definition) consists of the amino acid sequence of SEQ ID NO: 4412, CDR2 (AbM definition) consists of the amino acid sequence of SEQ ID NO: 4470, CDR3 (AbM definition) consists of the amino acid sequence of SEQ ID NO: 4524; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4524; or in which
CDR1 (AbM definition) consists of the amino acid sequence of SEQ ID NO: 4412, CDR2 (AbM definition) consists of the amino acid sequence of SEQ ID NO: 4470, CDR3 (AbM definition) consists of the amino acid sequence of SEQ ID NO: 4531; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4531; or in which
CDR1 (AbM definition) consists of the amino acid sequence of SEQ ID NO: 4412, CDR2 (AbM definition) consists of the amino acid sequence of SEQ ID NO: 4470, CDR3 (AbM definition) consists of the amino acid sequence of SEQ ID NO: 4526; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4526; or in which
CDR1 (AbM definition) consists of the amino acid sequence of SEQ ID NO: 4417, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4527; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4531; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4555; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4533; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4532; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4528; or in which
CDR1 (AbM definition) consists of the amino acid sequence of SEQ ID NO: 4412, CDR2 (AbM definition) consists of the amino acid sequence of SEQ ID NO: 4477 CDR3 (AbM definition) consists of the amino acid sequence of SEQ ID NO: 4527; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4605; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4604; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4601; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4603; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4584; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4582; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4555; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4533; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4532; or in which
CDR1 (AbM definition) consists of the amino acid sequence of SEQ ID NO: 4411, CDR2 (AbM definition) consists of the amino acid sequence of SEQ ID NO: 4481, CDR3 (AbM definition) consists of the amino acid sequence of SEQ ID NO: 4560; or in CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4593; or in which
CDR1 (AbM definition) consists of the amino acid sequence of SEQ ID NO: 4412, CDR2 (AbM definition) consists of the amino acid sequence of SEQ ID NO: 4470, CDR3 (AbM definition) consists of the amino acid sequence of SEQ ID NO: 4593; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4582; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4529; or in which
CDR1 (AbM definition) consists of the amino acid sequence of SEQ ID NO: 4429, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4433, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4559; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4547; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4561; or in which
CDR1 (AbM definition) consists of the amino acid sequence of SEQ ID NO: 4423, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4432, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4413, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4571; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4562; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4567; or in CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4563; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4564; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4569; or in which
CDR1 (AbM definition) consists of the amino acid sequence of SEQ ID NO: 4428, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4426, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4431, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4415, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4607; or in which
CDR1 (AbM definition) consists of the amino acid sequence of SEQ ID NO: 4416, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4422, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4410, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4434, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4435, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4436, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4430, CDR2 (AbM definition) consists of the amino acid sequence of SEQ ID NO: 4481, CDR3 (AbM definition) consists of the amino acid sequence of SEQ ID NO: 4560; or in CDR1 (AbM definition) consists of the amino acid sequence of SEQ ID NO: 4414, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4420, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4427, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4424, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4425, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4409, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4419, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4418, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4421, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4584; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4597; or in which
CDR1 (AbM definition) consists of the amino acid sequence of SEQ ID NO: 4412, CDR2 (AbM definition) consists of the amino acid sequence of SEQ ID NO: 4490, CDR3 (AbM definition) consists of the amino acid sequence of SEQ ID NO: 4586; or in which
CDR1 (AbM definition) consists of the amino acid sequence of SEQ ID NO: 4412, CDR2 (AbM definition) consists of the amino acid sequence of SEQ ID NO: 4493, CDR3 (AbM definition) consists of the amino acid sequence of SEQ ID NO: 4572; or in which
CDR1 (AbM definition) consists of the amino acid sequence of SEQ ID NO: 4408, CDR2 (AbM definition) consists of the amino acid sequence of SEQ ID NO: 4492, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4492, CDR3 (AbM definition) consists of the amino acid sequence of SEQ ID NO: 4572; or in CDR1 (AbM definition) consists of the amino acid sequence of SEQ ID NO: 4408, CDR2 (AbM definition) consists of the amino acid sequence of SEQ ID NO: 4491, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4491, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4466, CDR3 (AbM definition) consists of the amino acid sequence of SEQ ID NO: 4586; or in which
CDR1 (AbM definition) consists of the amino acid sequence of SEQ ID NO: 4412, CDR2 (AbM definition) consists of the amino acid sequence of SEQ ID NO: 4467, CDR3 (AbM definition) consists of the amino acid sequence of SEQ ID NO: 4586; or in which
CDR1 (AbM definition) consists of the amino acid sequence of SEQ ID NO: 4408, CDR2 (AbM definition) consists of the amino acid sequence of SEQ ID NO: 4467, CDR3 (AbM definition) consists of the amino acid sequence of SEQ ID NO: 4586; or in which
CDR1 (AbM definition) consists of the amino acid sequence of SEQ ID NO: 4408, CDR2 (AbM definition) consists of the amino acid sequence of SEQ ID NO: 4489, CDR3 (AbM definition) consists of the amino acid sequence of SEQ ID NO: 4572; or in which
CDR1 (AbM definition) consists of the amino acid sequence of SEQ ID NO: 4408, CDR2 (AbM definition) consists of the amino acid sequence of SEQ ID NO: 4466, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4467, CDR3 (AbM definition) consists of the amino acid sequence of SEQ ID NO: 4572; or in which
CDR1 (AbM definition) consists of the amino acid sequence of SEQ ID NO: 4408, CDR2 (AbM definition) consists of the amino acid sequence of SEQ ID NO: 4490, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4587; or in which
CDR1 (AbM definition) consists of the amino acid sequence of SEQ ID NO: 4412, CDR2 (AbM definition) consists of the amino acid sequence of SEQ ID NO: 4489, CDR3 (AbM definition) consists of the amino acid sequence of SEQ ID NO: 4572; or in which
CDR1 (AbM definition) consists of the amino acid sequence of SEQ ID NO: 4408, CDR2 (AbM definition) consists of the amino acid sequence of SEQ ID NO: 4467, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4468, CDR3 (AbM definition) consists of the amino acid sequence of SEQ ID NO: 4572; or in which
CDR1 (AbM definition) consists of the amino acid sequence of SEQ ID NO: 4408, CDR2 (AbM definition) consists of the amino acid sequence of SEQ ID NO: 4490, CDR3 (AbM definition) consists of the amino acid sequence of SEQ ID NO: 4586; or in which
CDR1 (AbM definition) consists of the amino acid sequence of SEQ ID NO: 4408, CDR2 (AbM definition) consists of the amino acid sequence of SEQ ID NO: 4464, CDR3 (AbM definition) consists of the amino acid sequence of SEQ ID NO: 4586; or in CDR1 (AbM definition) consists of the amino acid sequence of SEQ ID NO: 4412, CDR2 (AbM definition) consists of the amino acid sequence of SEQ ID NO: 4463, CDR3 (AbM definition) consists of the amino acid sequence of SEQ ID NO: 4586; or in which
CDR1 (AbM definition) consists of the amino acid sequence of SEQ ID NO: 4408, CDR2 (AbM definition) consists of the amino acid sequence of SEQ ID NO: 4464, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4464, CDR3 (AbM definition) consists of the amino acid sequence of SEQ ID NO: 4572; or in which
CDR1 (AbM definition) consists of the amino acid sequence of SEQ ID NO: 4408, CDR2 (AbM definition) consists of the amino acid sequence of SEQ ID NO: 4463, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4572; or in which
CDR1 (AbM definition) consists of the amino acid sequence of SEQ ID NO: 4408, CDR2 (AbM definition) consists of the amino acid sequence of SEQ ID NO: 4493, CDR3 (AbM definition) consists of the amino acid sequence of SEQ ID NO: 4586; or in which
CDR1 (AbM definition) consists of the amino acid sequence of SEQ ID NO: 4412, CDR2 (AbM definition) consists of the amino acid sequence of SEQ ID NO: 4493, CDR3 (AbM definition) consists of the amino acid sequence of SEQ ID NO: 4586; or in which
CDR1 (AbM definition) consists of the amino acid sequence of SEQ ID NO: 4408, CDR2 (AbM definition) consists of the amino acid sequence of SEQ ID NO: 4492, CDR3 (AbM definition) consists of the amino acid sequence of SEQ ID NO: 4586; or in which
CDR1 (AbM definition) consists of the amino acid sequence of SEQ ID NO: 4408, CDR2 (AbM definition) consists of the amino acid sequence of SEQ ID NO: 4491, CDR3 (AbM definition) consists of the amino acid sequence of SEQ ID NO: 4586; or in which
CDR1 (AbM definition) consists of the amino acid sequence of SEQ ID NO: 4412, CDR2 (AbM definition) consists of the amino acid sequence of SEQ ID NO: 4492, CDR3 (AbM definition) consists of the amino acid sequence of SEQ ID NO: 4586; or in which
CDR1 (AbM definition) consists of the amino acid sequence of SEQ ID NO: 4408, CDR2 (AbM definition) consists of the amino acid sequence of SEQ ID NO: 4489, CDR3 (AbM definition) consists of the amino acid sequence of SEQ ID NO: 4586; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4588; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4589; or in which
CDR1 (AbM definition) consists of the amino acid sequence of SEQ ID NO: 4408, CDR2 (AbM definition) consists of the amino acid sequence of SEQ ID NO: 4466, CDR3 (AbM definition) consists of the amino acid sequence of SEQ ID NO: 4586; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4590; or in CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4531; or in which
CDR1 (AbM definition) consists of the amino acid sequence of SEQ ID NO: 4412, CDR2 (AbM definition) consists of the amino acid sequence of SEQ ID NO: 4488, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4601; or in which
CDR1 (AbM definition) consists of the amino acid sequence of SEQ ID NO: 4412, CDR2 (AbM definition) consists of the amino acid sequence of SEQ ID NO: 4470, CDR3 (AbM definition) consists of the amino acid sequence of SEQ ID NO: 4582; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4565; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4596; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4593; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4595; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4584; or in which
CDR1 (AbM definition) consists of the amino acid sequence of SEQ ID NO: 4412, CDR2 (AbM definition) consists of the amino acid sequence of SEQ ID NO: 4470, CDR3 (AbM definition) consists of the amino acid sequence of SEQ ID NO: 4584; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4582; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4555; or in which
CDR1 (AbM definition) consists of the amino acid sequence of SEQ ID NO: 4412, CDR2 (AbM definition) consists of the amino acid sequence of SEQ ID NO: 4470, CDR3 (AbM definition) consists of the amino acid sequence of SEQ ID NO: 4555; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4570; or in which
CDR1 (AbM definition) consists of the amino acid sequence of SEQ ID NO: 4412, CDR2 (AbM definition) consists of the amino acid sequence of SEQ ID NO: 4470, CDR3 (AbM definition) consists of the amino acid sequence of SEQ ID NO: 4572; or in CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4533; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4532; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4529; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4604; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4605; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4526; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4557; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4526; or in which
CDR1 (AbM definition) consists of the amino acid sequence of SEQ ID NO: 4412, CDR2 (AbM definition) consists of the amino acid sequence of SEQ ID NO: 4485, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4597; or in which
CDR1 (AbM definition) consists of the amino acid sequence of SEQ ID NO: 4412, CDR2 (AbM definition) consists of the amino acid sequence of SEQ ID NO: 4484, CDR3 (AbM definition) consists of the amino acid sequence of SEQ ID NO: 4572; or in which
CDR1 (AbM definition) consists of the amino acid sequence of SEQ ID NO: 4408, CDR2 (AbM definition) consists of the amino acid sequence of SEQ ID NO: 4485, CDR3 (AbM definition) consists of the amino acid sequence of SEQ ID NO: 4586; or in which
CDR1 (AbM definition) consists of the amino acid sequence of SEQ ID NO: 4412, CDR2 (AbM definition) consists of the amino acid sequence of SEQ ID NO: 4485, CDR3 (AbM definition) consists of the amino acid sequence of SEQ ID NO: 4586; or in which
CDR1 (AbM definition) consists of the amino acid sequence of SEQ ID NO: 4408, CDR2 (AbM definition) consists of the amino acid sequence of SEQ ID NO: 4488, CDR3 (AbM definition) consists of the amino acid sequence of SEQ ID NO: 4586; or in which
CDR1 (AbM definition) consists of the amino acid sequence of SEQ ID NO: 4412, CDR2 (AbM definition) consists of the amino acid sequence of SEQ ID NO: 4488, CDR3 (AbM definition) consists of the amino acid sequence of SEQ ID NO: 4586; or in CDR1 (AbM definition) consists of the amino acid sequence of SEQ ID NO: 4408, CDR2 (AbM definition) consists of the amino acid sequence of SEQ ID NO: 4486, CDR3 (AbM definition) consists of the amino acid sequence of SEQ ID NO: 4586; or in which
CDR1 (AbM definition) consists of the amino acid sequence of SEQ ID NO: 4412, CDR2 (AbM definition) consists of the amino acid sequence of SEQ ID NO: 4486, CDR3 (AbM definition) consists of the amino acid sequence of SEQ ID NO: 4586; or in which
CDR1 (AbM definition) consists of the amino acid sequence of SEQ ID NO: 4408, CDR2 (AbM definition) consists of the amino acid sequence of SEQ ID NO: 4481, CDR3 (AbM definition) consists of the amino acid sequence of SEQ ID NO: 4586; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4586; or in which
CDR1 (AbM definition) consists of the amino acid sequence of SEQ ID NO: 4408, CDR2 (AbM definition) consists of the amino acid sequence of SEQ ID NO: 4485, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4486, CDR3 (AbM definition) consists of the amino acid sequence of SEQ ID NO: 4572; or in which
CDR1 (AbM definition) consists of the amino acid sequence of SEQ ID NO: 4408, CDR2 (AbM definition) consists of the amino acid sequence of SEQ ID NO: 4488, CDR3 (AbM definition) consists of the amino acid sequence of SEQ ID NO: 4572; or in which
CDR1 (AbM definition) consists of the amino acid sequence of SEQ ID NO: 4408, CDR2 (AbM definition) consists of the amino acid sequence of SEQ ID NO: 4486, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4487, CDR3 (AbM definition) consists of the amino acid sequence of SEQ ID NO: 4572; or in which
CDR1 (AbM definition) consists of the amino acid sequence of SEQ ID NO: 4408, CDR2 (AbM definition) consists of the amino acid sequence of SEQ ID NO: 4481, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4603; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4558; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4568; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4566; or in which
CDR1 (AbM definition) consists of the amino acid sequence of SEQ ID NO: 4456, CDR2 (AbM definition) consists of the amino acid sequence of SEQ ID NO: 4481, CDR3 (AbM definition) consists of the amino acid sequence of SEQ ID NO: 4560; or in CDR1 (AbM definition) consists of the amino acid sequence of SEQ ID NO: 4458, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4451, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4455, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4448, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4392, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4401, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4446, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4450, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4457, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4398, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4388, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4400, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4399, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4397, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4389, CDR2 (AbM definition) consists of the amino acid sequence of SEQ ID NO: 4481, CDR3 (AbM definition) consists of the amino acid sequence of SEQ ID NO: 4560; or in CDR1 (AbM definition) consists of the amino acid sequence of SEQ ID NO: 4453, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4393, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4396, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4391, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4407, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4442, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4404 CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4405, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4445, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4440, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4441, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4443, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4403, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4438, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4459, CDR2 (AbM definition) consists of the amino acid sequence of SEQ ID NO: 4481, CDR3 (AbM definition) consists of the amino acid sequence of SEQ ID NO: 4560; or in CDR1 (AbM definition) consists of the amino acid sequence of SEQ ID NO: 4452, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4447, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4454, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4449, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4460, CDR2 (AbM definition) consists of the amino acid sequence of SEQ ID NO: 4482, CDR3 (AbM definition) consists of the amino acid sequence of SEQ ID NO: 4560; or in which
CDR1 (AbM definition) consists of the amino acid sequence of SEQ ID NO: 4395, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4394 CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4437 CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4556; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4554; or in which
CDR1 (AbM definition) consists of the amino acid sequence of SEQ ID NO: 4408, CDR2 (AbM definition) consists of the amino acid sequence of SEQ ID NO: 4463, CDR3 (AbM definition) consists of the amino acid sequence of SEQ ID NO: 4586; or in which
CDR1 (AbM definition) consists of the amino acid sequence of SEQ ID NO: 4408, CDR2 (AbM definition) consists of the amino acid sequence of SEQ ID NO: 4493, CDR3 (AbM definition) consists of the amino acid sequence of SEQ ID NO: 4572; or in which
CDR1 (AbM definition) consists of the amino acid sequence of SEQ ID NO: 4412, CDR2 (AbM definition) consists of the amino acid sequence of SEQ ID NO: 4465, CDR3 (AbM definition) consists of the amino acid sequence of SEQ ID NO: 4586; or in which
CDR1 (AbM definition) consists of the amino acid sequence of SEQ ID NO: 4406, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4472, CDR3 (AbM definition) consists of the amino acid sequence of SEQ ID NO: 4560; or in CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4552; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4551; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4548; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4541; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4553; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4544; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4550; or in which
CDR1 (AbM definition) consists of the amino acid sequence of SEQ ID NO: 4390, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4546; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4549; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4540; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4545; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4542; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4539; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4534; or in CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4522; or in which
CDR1 (AbM definition) consists of the amino acid sequence of SEQ ID NO: 4402, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4591; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4602; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4525; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4576; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4575; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4585; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4578; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4574; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4579; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4577; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4580; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4581; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4583; or in CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4573; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4536; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4543; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4538; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4523; or in which
CDR1 (AbM definition) consists of the amino acid sequence of SEQ ID NO: 4444, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4470, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4471, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4474, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4483, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4477, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4479, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4476, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4473, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4480, CDR3 (AbM definition) consists of the amino acid sequence of SEQ ID NO: 4560; or in CDR1 (AbM definition) consists of the amino acid sequence of SEQ ID NO: 4412, CDR2 (AbM definition) consists of the amino acid sequence of SEQ ID NO: 4469, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4475, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4606; or in which
CDR1 (AbM definition) consists of the amino acid sequence of SEQ ID NO: 4439, CDR2 (AbM definition) consists of the amino acid sequence of SEQ ID NO: 4481, CDR3 (AbM definition) consists of the amino acid sequence of SEQ ID NO: 4560; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4598; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4537; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4599; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4600; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4530; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4594; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4535; or in which
CDR1 (AbM definition) consists of the amino acid sequence of SEQ ID NO: 4412, CDR2 (AbM definition) consists of the amino acid sequence of SEQ ID NO: 4481, CDR3 (AbM definition) consists of the amino acid sequence of SEQ ID NO: 4592.

91-93. (canceled)

94. A polypeptide or construct that comprises or essentially consists of one or more ISVDs according to claim 76, and optionally further comprises one or more other groups, residues, moieties or binding units, optionally linked via one or more linkers.

95. A nucleic acid that encodes an ISVD according to claim 76, optionally wherein a (non-human) host or host cell expresses or that under suitable circumstances is capable of expressing and/or that comprises the nucleic acid encoding the ISVD.

96. (canceled)

97. A method for producing an ISVD according to claim 76, at least comprising the steps of:
a) expressing, in a suitable (non-human) host organism or a host cell or in another suitable expression system, a nucleic acid encoding the ISVD;
optionally followed by:
b) isolating and/or purifying the ISVD; or
a method for producing an ISVD according to claim 76, said method at least comprising the steps of:
a) cultivating and/or maintaining a (non-human) host or host cell under conditions that are such that said non-human host or host cell expresses and/or produces at least one ISVD according to claim 76;
optionally followed by:
b) isolating and/or purifying the ISVD according to claim 76.

98. (canceled)

99. A composition comprising at least one ISVD according to claim 76, optionally wherein the composition is a pharmaceutical composition.

100-104. (canceled)

105. A method for the diagnosis, prevention and/or treatment of at least one disease and/or disorder, comprising the administration, to a subject in need thereof, of a pharmaceutically active amount of an ISVD according to claim 99, optionally wherein the at least one disease and/or disorder is cancer.

106. (canceled)

107. A bispecific binding protein comprising:
(a) a first binding moiety comprising at least three GITR ISVDs directly linked to each other and wherein one GITR ISVD is linked to the N-terminal end of a first IgG;
(b) a second binding moiety comprising at least two PD-1 ISVDs directly linked to each other and wherein one PD-1 ISVD is linked to the N-terminal end of a second IgG; and
wherein each the first and second IgG1 domains heterodimerize to form a Fc domain or variant thereof, optionally wherein the first binding moiety consists of at least three GITR ISVDs, and/or the second moiety consists of at least two PD-1 ISVDs; or
a bispecific binding protein comprising:
(a) a first binding moiety comprising at least three GITR ISVDs directly linked to each other and wherein one GITR ISVD is linked to the C-terminal end of a first and a second IgG;
(b) a second binding moiety comprising at least two PD-1 ISVDs wherein each PD-1 ISVD is directly linked to the N-terminal end of the first and the second IgG; and
wherein each the first and second IgG1 domains form a Fc domain, optionally wherein the first binding moiety consists of at least six GITR ISVDs.

108-111. (canceled)

112. The bispecific binding protein of claim 107,
wherein each GITR ISVD is an agonist of GITR;
wherein the second moiety consists of at least two PD-1 ISVDs;
wherein each PD-1 ISVD is an antagonist of PD-1;
wherein each GITR ISVD is an agonist of GITR and each PD-1 ISVD is an antagonist of PD-1;
wherein each GITR ISVD is linked via a poly-Glycine-Serine (G4S) n (SEQ ID NO: 4734) linker;
wherein each PD-1 ISVD is linked via a poly-Glycine-Serine (G4S) n (SEQ ID NO: 4734) linker;
wherein n equals 1, 2, 3, 4, or 5;
wherein n equals 1;
wherein the one GITR ISVD is linked to the C-terminal end of the first and the second IgG via a poly-Glycine-Serine (G4S) n (SEQ ID NO: 4734) linker;
wherein n equals 1, 2, 3, 4, or 5;
wherein n equals 4;
wherein at least one GITR ISVD comprises the three following CDR sequences:
ISVD-CDR1:
(SEQ ID NO: 4720)
GTFFSIDSMA;
ISVD-CDR2:
(SEQ ID NO: 4721)
AITGGGSPN;
and
ISVD-CDR3:
(SEQ ID NO: 4722)
EGQAGWGTALLDY;
wherein the at least one PD-1 ISVD comprises one of the three following CDR sequences:
ISVD-CDR1:
(SEQ ID NO: 4723 or SEQ ID NO: 4726
GFTLDYYVIG or GRMHSINAMA;
ISVD-CDR2:
(SEQ ID NO: 4724 or SEQ ID NO: 4727)
CLSSNGDRIN or LISWGNGITY
ISVD-CDR3:
(SEQ ID NO: 4725 or SEQ ID NO: 4728)
GTSTTVRDMCGIMYLYDY or SYGSSWYDS;
wherein the at least one GITR ISVD specific variable domain has a sequence identity of at least 80% with an amino acid sequence selected from the group consisting of SEQ ID NO: 4714 and 4715; and/or
wherein the at least one PD-1 ISVD specific variable domain has a sequence identity of at least 80% with an amino acid sequence selected from the group consisting of SEQ ID NO: 4716, 4717, 4718 and 4719.

113-126. (canceled)

127. A compound or construct that comprises the bispecific binding protein claim 107 that further comprises one or more other groups, residues, moieties or binding units, optionally linked via one or more peptide linker(s).

128. A nucleic acid encoding the bispecific binding protein according to claim 107 or an expression vector comprising a nucleic acid encoding the bispecific binding protein.

129. (canceled)

130. A host or host cell comprising a nucleic acid or an expression vector according to claim 128.

131. A pharmaceutical composition comprising the bispecific binding protein according to claim 107 and a pharmaceutically acceptable carrier.

132. A method of treating
a neoplastic disorder comprising administering the bispecific binding protein according to claim 107 to a subject, optionally wherein administration of the binding protein results in tumor growth inhibition by 1, 2, 3, or 4-fold relative to a reference binding protein;
a method of treating an infection comprising administering the bispecific binding protein according to claim 107 to a subject; and/or
a method of treating a T cell, B cell, or natural killer cell mediated disease comprising administering the bispecific binding protein according to claim 107 to a subject.

133-135. (canceled)
